(19) United States
(12) Patent Application Publication
(10) Pub. No.: US 2023/0056182 A1
(43) Pub. Date: Feb. 23, 2023

(54) USE OF ADENO-ASSOCIATED VIRAL VECTORS TO CORRECT GENE DEFECTS/ EXPRESS PROTEINS IN HAIR CELLS AND SUPPORTING CELLS IN THE INNER EAR

(71) Applicant: Akouos, Inc., Boston, MA (US)

(72) Inventors: Emmanuel John Simons, Brookline, MA (US); Robert Ng, Newton, MA (US); Danielle R. Lenz, Brookline, MA (US); Michelle Valero, Revere, MA (US)

(21) Appl. No.: 17/291,931

(22) PCT Filed: Nov. 7, 2019
(86) PCT No.: PCT/US2019/060328

§ 371 Date: May 6, 2021

Related U.S. Application Data
(60) Provisional application No. 62/756,953, filed on Nov. 7, 2018.
(60) Provisional application No. 62/888,093, filed on Aug. 16, 2019.

(51) Int. Cl. A61K 48/00; A61P 27/16; C12N 15/86

(52) U.S. Cl. CPC A61K 48/0075; C12N 15/86; A61P 27/16

(57) ABSTRACT

Provided herein are compositions that include at least two different nucleic acid vectors that, when introduced into a primate cell, the at least two different vectors undergo con catamerization or homologous recombination with each other, thereby forming a recombined nucleic acid that encodes a full-length target protein (e.g., a supporting cell target protein or a hair cell target protein). Also provided are compositions that include a single AAV vector that, when introduced into a primate cell, a nucleic acid encoding a full-length target protein (e.g., a supporting cell target protein or a hair cell target protein) is generated at the locus of the supporting cell target gene, and the primate expresses the target protein (e.g., a supporting cell target protein or a hair cell target protein).

### CROSS-REFERENCE TO RELATED APPLICATIONS

This application claims priority to U.S. Provisional Patent Application Ser. No. 62/756,953, filed Nov. 7, 2018, and U.S. Provisional Patent Application Ser. No. 62/888,093, filed Aug. 16, 2019; the entire contents of which are herein incorporated by reference.

### TECHNICAL FIELD

The present disclosure relates generally to the use of nucleic acids to treat hearing loss in a primate.

### BACKGROUND OF THE INVENTION

Hearing loss can be conductive (arising from the ear canal or middle ear), sensorineural (arising from the inner ear or auditory nerve), or mixed. Most forms of non-syndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present invention is based on the discovery that a composition including at least two different nucleic acid vectors, wherein: each of the at least two different adeno-associated virus (AAV) vectors includes a coding sequence that encodes a different portion of a supporting cell target protein, each of the encoded portions being at least 30 amino acid residues in length, wherein the amino acid sequence of each of the encoded portions may optionally partially overlap with the amino acid sequence of a different one of the encoded portions; no single vector of the at least two different vectors encodes the full-length supporting cell target protein; at least one of the coding sequences includes a nucleotide sequence spanning two neighboring exons of the supporting cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length supporting cell target protein.

Provided herein are compositions that include at least two different nucleic acid vectors, where: each of the at least two different adeno-associated virus (AAV) vectors includes a coding sequence that encodes a different portion of a supporting cell target protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length supporting cell target protein; at least one of the coding sequences includes a nucleotide sequence spanning two neighboring exons of the supporting cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length supporting cell target protein.

In some embodiments of any of the compositions described herein, the amino acid sequence of one of the encoded portions overlaps with the amino acid sequence of a different one of the encoded portions. In some embodiments of any of the compositions described herein, the amino acid sequence of each of the encoded portions partially overlaps with the amino acid sequence of a different encoded portion. In some embodiments of any of the compositions described herein, the overlapping amino acid sequence is between 30 amino acid residues to about 390 amino acid residues in length.

In some embodiments of any of the compositions described herein, each of the at least two different vectors includes a different segment of an intron, where the intron includes the nucleotide sequence of an intron that is present in a supporting cell target genomic DNA, and where the two different segments overlap in sequence by at least 100 nucleotides. In some embodiments of any of the compositions described herein, the two different segments overlap in sequence by about 30 nucleotides to about 390 nucleotides. In some embodiments of any of the compositions described herein, the nucleotide sequence of each of the at least two different vectors is between about 30 nucleotides to about 390 nucleotides in length. In some embodiments of any of the compositions described herein, the nucleotide sequence of each of the at least two different vectors is between 30 nucleotides to 340 nucleotides in length.

In some embodiments of any of the compositions described herein, the number of different vectors in the composition is two. In some embodiments of any of the compositions described herein, a first of the two different vectors includes a coding sequence that encodes an N-terminal portion of the supporting cell target protein. In some embodiments of any of the compositions described herein, the N-terminal portion of the supporting cell target protein is between about 30 amino acids to about 390 amino acids in length. In some embodiments of any of the compositions described herein, the N-terminal portion of the supporting cell target protein is between about 30 amino acids to about 340 amino acids in length. In some embodiments of any of the compositions described herein, the first vector further includes one or both of a promoter and a Kozak sequence. In some embodiments of any of the compositions described herein, the first vector includes a promoter that is an inducible promoter, a constitutive promoter, or a tissue-specific promoter.

In some embodiments of any of the compositions described herein, the second of the two different vectors includes a coding sequence that encodes a C-terminal portion of the supporting cell target protein. In some embodiments of any of the compositions described herein, the C-terminal portion of the supporting cell target protein is between 30 amino acids to about 390 amino acids in length. In some embodiments of any of the compositions described herein, the C-terminal portion of the supporting cell target protein is between 30 amino acids to about 340 amino acids in length. In some embodiments of any of the compositions described herein, the second vector further includes a poly(dA) sequence.

Also provided herein are compositions that include two different nucleic acid vectors, where: a first nucleic acid vector of the two different nucleic acid vectors includes a promoter, a first coding sequence that encodes an N-terminal portion of a supporting cell target protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a supporting cell target protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length supporting cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length supporting cell target protein.

In some embodiments of any of the compositions described herein, at least one of the coding sequences includes a nucleotide sequence spanning two neighboring exons of a supporting cell target genomic DNA, and lacks an intronic sequence between the neighboring exons.

Also provided herein are compositions that include: a first nucleic acid vector including a promoter, a first coding sequence that encodes an N-terminal portion of a supporting cell target protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a supporting cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length supporting cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length supporting cell target protein.

In some embodiments of any of the compositions described herein, at least one of the coding sequences includes a nucleotide sequence spanning two neighboring exons of a supporting cell target genomic DNA, and lacks an intronic sequence between the neighboring exons. In some embodiments of any of the compositions described herein, the first or second detectable marker gene is alkaline phosphatase. In some embodiments of any of the compositions described herein, the first and second detectable marker genes are the same.

Also provided herein are compositions that include: a first nucleic acid vector including a promoter, a first coding sequence that encodes an N-terminal portion of a supporting cell target protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a supporting cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length supporting cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length supporting cell target protein.

In some embodiments of any of the compositions described herein, at least one of the coding sequences includes a nucleotide sequence spanning two neighboring exons of a supporting cell target genomic DNA, and lacks an intronic sequence between the two neighboring exons.

Also provided herein are compositions that include a single adeno-associated virus (AAV) vector, where the single AAV vector that includes a nucleic acid sequence that encodes a supporting cell target protein; and when introduced into a primate cell, a nucleic acid encoding a full-length supporting cell target protein is generated at the locus of the supporting cell target gene, and the primate expresses the supporting cell target protein.

In some embodiments of any of the compositions described herein, the supporting cell target gene is gap junction protein beta 2 (GJB2). In some embodiments of any of the compositions described herein, the supporting cell target gene is solute carrier family 26 member 4 (SLC26A4).

In some embodiments of any of the compositions described herein, the composition further includes a pharmaceutically acceptable excipient.

In some embodiments of any of the compositions described herein, the second nucleic acid vector further includes a destabilizing sequence.

In some embodiments of any of the compositions described herein, the second nucleic acid vector further includes a FKBP12 destabilizing sequence.

Also provided herein are kits including any of the compositions described herein. In some embodiments of any of the kits described herein, the kit further includes a pre-loaded syringe containing the composition.

Also provided herein are methods of correcting a supporting cell target gene defect in a supporting cell of an inner ear in a primate that include: administering to the inner ear of the primate a therapeutically effective amount of any of the compositions described herein, where the administering repairs the supporting cell target gene defect in the supporting cell of the inner ear of the primate.

Also provided herein are methods of increasing the expression level of a supporting cell target protein in a supporting cell of an inner ear of a primate that include: administering to the inner ear of the primate a therapeutically effective amount of any of the compositions described herein, where the administering results in an increase in the expression level of the supporting cell target protein in the supporting cell of the inner ear of the primate.

In some embodiments of any of the methods described herein, the primate has previously been determined to have a defective supporting cell target gene.

Also provided herein are methods of treating non-syndromic sensorineural hearing loss in a primate identified as having a defective supporting cell target gene that include: administering to the inner ear of the primate a therapeutically effective amount of any of the compositions described herein.

In some embodiments of any of the methods described herein, the method further includes, prior to the administering step, determining that the primate has a defective supporting cell target gene.

Also provided herein are methods of restoring hearing in a primate identified or diagnosed as having hearing loss that include: administering to the inner ear of the primate a therapeutically effective amount of any composition described herein.

In some embodiments of any of the methods described herein, the method further includes prior to the administering step, determining that the primate has a defective supporting cell target gene.

Also provided herein are methods of restoring synapses and/or preserving spiral ganglion nerves in a primate identified or diagnosed as having an inner ear disorder that include: administering to the inner ear of the primate a therapeutically effective amount of any composition described herein.

In some embodiments of any of the methods described herein, the method further includes prior to the administering step, determining that the primate has a defective supporting cell target gene.

Also provided herein are methods including administering to an inner ear of a primate a therapeutically effective amount of any composition described herein.

In some embodiments of any of the methods described herein, the primate has been previously identified as having a defective supporting cell target gene.

Also provided herein are compositions including at least two different nucleic acid vectors, wherein: each of the at least two different adeno-associated virus (AAV) vectors includes a coding sequence that encodes a different portion of a hair cell target protein, each of the encoded portions being at least 30 amino acid residues in length, wherein the amino acid sequence of each of the encoded portions may optionally partially overlap with the amino acid sequence of a different one of the encoded portions; no single vector of the at least two different vectors encodes the full-length hair cell target protein; at least one of the coding sequences includes a nucleotide sequence spanning two neighboring exons of the hair cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length hair cell target protein.

Provided herein are compositions that include at least two different nucleic acid vectors, where: each of the at least two different adeno-associated virus (AAV) vectors includes a coding sequence that encodes a different portion of a hair cell target protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length protein; at least one of the coding sequences includes a nucleotide sequence spanning two neighboring exons of the hair cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length hair cell target protein.

In some embodiments of any of the compositions described herein, the amino acid sequence of one of the encoded portions overlaps with the amino acid sequence of a different one of the encoded portions. In some embodiments of any of the compositions described herein, the amino acid sequence of each of the encoded portions partially overlaps with the amino acid sequence of a different encoded portion. In some embodiments of any of the compositions described herein, the overlapping amino acid sequence is between 30 amino acid residues to about 390 amino acid residues in length.

In some embodiments of any of the compositions described herein, each of the at least two different vectors includes a different segment of an intron, where the intron includes the nucleotide sequence of an intron that is present in a hair cell target genomic DNA, and where the two different segments overlap in sequence by at least 100 nucleotides. In some embodiments of any of the compositions described herein, the two different segments overlap in sequence by about 30 nucleotides to about 390 nucleotides. In some embodiments of any of the compositions described herein, the nucleotide sequence of each of the at least two different vectors is between about 30 nucleotides to about 390 nucleotides in length. In some embodiments of any of the compositions described herein, the nucleotide sequence of each of the at least two different vectors is between 30 nucleotides to 340 nucleotides in length.

In some embodiments of any of the compositions described herein, the number of different vectors in the composition is two. In some embodiments of any of the compositions described herein, a first of the two different vectors includes a coding sequence that encodes an N-terminal portion of the hair cell target protein. In some embodiments of any of the compositions described herein, the N-terminal portion of the hair cell target protein is between about 30 amino acids to about 390 amino acids in length. In some embodiments of any of the compositions described herein, the N-terminal portion of the hair cell target protein is between about 30 amino acids to about 340 amino acids in length. In some embodiments of any of the compositions described herein, the first vector further includes one or both of a promoter and a Kozak sequence. In some embodiments of any of the compositions described herein, the first vector includes a promoter that is an inducible promoter, a constitutive promoter, or a tissue-specific promoter.

In some embodiments of any of the compositions described herein, the second of the two different vectors includes a coding sequence that encodes a C-terminal portion of the hair cell target protein. In some embodiments of any of the compositions described herein, the C-terminal portion of the hair cell target protein is between 30 amino acids to about 390 amino acids in length. In some embodiments of any of the compositions described herein, the C-terminal portion of the hair cell target protein is between 30 amino acids to about 340 amino acids in length. In some embodiments of any of the compositions described herein, the second vector further includes a poly(dA) sequence.

Also provided herein are compositions that include two different nucleic acid vectors, where: a first nucleic acid vector of the two different nucleic acid vectors includes a promoter, a first coding sequence that encodes an N-terminal portion of a hair cell target protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a hair cell target protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length hair cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length hair cell target protein.

In some embodiments of any of the compositions described herein, at least one of the coding sequences includes a nucleotide sequence spanning two neighboring exons of a hair cell target genomic DNA, and lacks an intronic sequence between the neighboring exons.

Also provided herein are compositions that include: a first nucleic acid vector including a promoter, a first coding sequence that encodes an N-terminal portion of a hair cell target protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a hair cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length hair cell target protein.

In some embodiments of any of the compositions described herein, at least one of the coding sequences includes a nucleotide sequence spanning two neighboring exons of a hair cell target genomic DNA, and lacks an intronic sequence between the neighboring exons. In some embodiments of any of the compositions described herein, the first or second detectable marker gene is alkaline phosphatase. In some embodiments of any of the compositions described herein, the first and second detectable marker genes are the same.

Also provided herein are compositions that include: a first nucleic acid vector including a promoter, a first coding sequence that encodes an N-terminal portion of a hair cell target protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a hair cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length hair cell target protein.

In some embodiments of any of the compositions described herein, at least one of the coding sequences includes a nucleotide sequence spanning two neighboring exons of a hair cell target genomic DNA, and lacks an intronic sequence between the two neighboring exons.

Also provided herein are compositions that include a single adeno-associated virus (AAV) vector, where the single AAV vector that includes a nucleic acid sequence that encodes a hair cell target protein; and when introduced into a primate cell, a nucleic acid encoding a full-length hair cell target protein is generated at the locus of the hair cell target gene, and the primate expresses the hair cell target protein.

In some embodiments of any of the compositions described herein, the composition further includes a pharmaceutically acceptable excipient.

Also provided herein are kits including any of the compositions described herein. In some embodiments of any of the kits described herein, the kit further includes a pre-loaded syringe containing the composition.

Also provided herein are methods of correcting a hair cell target gene defect in a hair cell of an inner ear in a primate that include: administering to the inner ear of the primate a therapeutically effective amount of any of the compositions described herein, where the administering repairs the hair cell target gene defect in the hair cell of the inner ear of the primate.

Also provided herein are methods of increasing the expression level of a hair cell target protein in a hair cell of an inner ear of a primate that include: administering to the inner ear of the primate a therapeutically effective amount of any of the compositions described herein, where the administering results in an increase in the expression level of the hair cell target protein in the hair cell of the inner ear of the primate.

In some embodiments of any of the methods described herein, the primate has previously been determined to have a defective hair cell target gene.

Also provided herein are methods of treating non-syndromic sensorineural hearing loss in a primate identified as having a defective hair cell target gene that include: administering to the inner ear of the primate a therapeutically effective amount of any of the compositions described herein.

In some embodiments of any of the methods described herein, the method further includes, prior to the administering step, determining that the primate has a defective hair cell target gene.

Also provided herein are methods of restoring hearing in a primate identified or diagnosed as having hearing loss that include: administering to the inner ear of the primate a therapeutically effective amount of any composition described herein.

In some embodiments of any of the methods described herein, the method further includes prior to the administering step, determining that the primate has a defective hair cell target gene.

Also provided herein are methods of restoring synapses and/or preserving spiral ganglion nerves in a primate identified or diagnosed as having an inner ear disorder that include: administering to the inner ear of the primate a therapeutically effective amount of any composition described herein.

In some embodiments of any of the methods described herein, the method further includes prior to the administering step, determining that the primate has a defective hair cell target gene.

Also provided herein are methods including administering to an inner ear of a primate a therapeutically effective amount of any composition described herein.

In some embodiments of any of the methods described herein, the primate has been previously identified as having a defective hair cell target gene.

The term “a” and “an” refers to one or to more than one (i.e., at least one) of the grammatical object of the article. By way of example, “an element” encompasses one element and more than one element.

The term “supporting cell target protein” means a protein that is expressed in a supporting cell in a primate and functionally contributes, at least in part, to the hearing in the primate. Non-limiting examples of supporting cell target proteins include GJB2 and SLC26A4.

The term “mutation in a supporting cell target gene” refers to a modification in a wildtype supporting cell target gene that results in the production of a supporting cell target protein having one or more of: a deletion in one or more amino acids, one or more amino acid substitutions, and one or more amino acid insertions as compared to the wildtype supporting cell target protein, and/or results in a decrease in the expressed level of the encoded supporting cell target protein in a primate cell as compared to the expressed level of the encoded supporting cell target protein in a primate cell not having a mutation. In some embodiments, a mutation can result in the production of a supporting cell target protein having a deletion in one or more amino acids (e.g., 2, 3, 4, 5, 6, 7, 8, 9, 10, 11, 12, 13, 14, 15 16, 17, 18, 19, or 20 amino acids). In some embodiments, the mutation can result in a frameshift in the supporting cell target gene. The term “hair cell target protein” means a protein that is expressed in a hair cell in a primate and functionally contributes, at least in part, to the hearing in the primate. Non-limiting examples of hair cell target proteins include tectorin alpha (TECTA), unconventional myson-15 (MYO15A), myosin MA (MYO3A), myosin VI (MYO6), myosin VIIA (MYO7A), cadherin related 23 (CDH23), collagen type XI alpha 2 chain (COL11A2), lipoxygenase homology domains 1 (LOXHD1), protocadherin related 15 (PCDH15), whirlin (WHRN), an otoferlin (OTOF) gene, a trio rho guanine nucleotide exchange factor (TRIO) and f-actin binding protein (TRIOBP) gene, a stereocilin (STRC) gene, a pejvakin (PJVK) gene, a norrin cysteine knot growth factor (NDP) gene, a clarin 1 (CLRN1) gene, a transmembrane channel-like 1 (TMC1) gene, a vascular endothelial growth factor (VEGF) gene, and a heat shock protein family A (Hsp70) member 1A (HSPA1A) gene.

The term “mutation in a hair cell target gene” refers to a modification in a wildtype hair cell target gene that results in the production of a hair cell target protein having one or more of: a deletion in one or more amino acids, one or more amino acid substitutions, and one or more amino acid insertions as compared to the wildtype hair cell target protein, and/or results in a decrease in the expressed level of the encoded hair cell target protein in a primate cell as compared to the expressed level of the encoded hair cell target protein in a primate cell not having a mutation. In some embodiments, a mutation can result in the production of a hair cell target protein having a deletion in one or more amino acids (e.g., 2, 3, 4, 5, 6, 7, 8, 9, 10, 11, 12, 13, 14, 15 16, 17, 18, 19, or 20 amino acids). In some embodiments, the mutation can result in a frameshift in the hair cell target gene.

The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding wildtype protein) that may or may not be functional. In some embodiments, the mutation can result in the loss (or a decrease in the level) of expression of hair cell target mRNA or hair cell target protein or both the mRNA and protein. In some embodiments, the mutation can result in the production of an altered hair cell target protein having a loss or decrease in one or more biological activities (functions) as compared to a wildtype hair cell target protein. In some embodiments, the mutation can result in the loss (or a decrease in the level) of expression of supporting cell target mRNA or supporting cell target protein or both the mRNA and protein. In some embodiments, the mutation can result in the production of an altered supporting cell target protein having a loss or decrease in one or more biological activities (functions) as compared to a wildtype supporting cell target protein.

In some embodiments, the mutation is an insertion of one or more nucleotides into a hair cell target gene. In some embodiments, the mutation is in a regulatory sequence of the hair cell target gene, i.e., a portion of the gene that is not coding sequence. In some embodiments, a mutation in a regulatory sequence may be in a promoter or enhancer region and prevent or reduce the proper transcription of the hair cell target gene. In some embodiments, the mutation is an insertion of one or more nucleotides into a supporting cell target gene. In some embodiments, the mutation is in a regulatory sequence of the supporting cell target gene, i.e., a portion of the gene that is not coding sequence. In some embodiments, a mutation in a regulatory sequence may be in a promoter or enhancer region and prevent or reduce the proper transcription of the supporting cell target gene. The term “conservative mutation” refers to a mutation that does not change the amino acid encoded at the site of the mutation (due to codon degeneracy).

Modifications can be introduced into a nucleotide sequence by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine, and isoleucine), and aromatic side chains (e.g., tyrosine, phenylalanine, tryptophan, and histidine).

Unless otherwise specified, a “nucleotide sequence encoding an amino acid sequence” includes all nucleotide sequences that are degenerate versions of each other and thus encode the same amino acid sequence.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” primate cell is one that has been transfected, transformed or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell (e.g., a primate cell) is lost upon multiple rounds of cell division.

The term “primate” is intended to include any primate (e.g., a human, a non-human primate (e.g., simian (e.g., a monkey (e.g., a marmoset, a baboon, or a macaque), or an ape (e.g., a gorilla, a gibbon, an orangutan, or a chimpanzee). In some embodiments, the primate has or is at risk of having hearing loss. In some embodiments, the primate has been previously identified as having a mutation in a hair cell target gene (e.g., an OTOF gene, a TRIOBP gene, a STRC gene, a PJVK gene, a NDP gene, a CLRN1 gene, a TMC1 gene, a VEGF gene, or a HSPA1A gene). In some embodiments, the primate has been previously identified as having a mutation in a supporting cell target gene (e.g., a GJB2 gene or a SLC26A4 gene). In some embodiments, the primate has been previously identified as having a mutation in a GJB2 gene. In some embodiments, the primate has been previously identified as having a mutation in a SLC26A4 gene. In some embodiments, the primate has been identified as having a mutation in a hair cell target gene (e.g., an OTOF gene, a TRIOBP gene, a STRC gene, a PJVK gene, a NDP gene, a CLRN1 gene, a TMC1 gene, a VEGF gene, or a HSPA1A gene) and has been diagnosed with hearing loss. In some embodiments, the primate has been identified as having a mutation in a supporting cell target gene (e.g., a GBJ2 gene or a SLC26A4 gene) and has been diagnosed with hearing loss. In some embodiments, the primate has been identified as having hearing loss.

A treatment is “therapeutically effective” when it results in a reduction in one or more of the number, severity, and frequency of one or more symptoms of a disease state (hearing loss, e.g., non-syndromic sensorineural hearing loss or syndromic sensorineural hearing loss) in a primate. In some embodiments, a therapeutically effective amount of a composition can result in an increase in the expression level of an active target protein (e.g., a supporting cell target protein (e.g., a wildtype, full-length supporting cell target protein or a variant of a supporting cell target protein that has the desired activity) (e.g., as compared to the expression level prior to treatment with the composition) or a hair cell target protein (e.g., a wildtype, full-length hair cell target protein or a variant of a hair cell target protein that has the desired activity) (e.g., as compared to the expression level prior to treatment with the composition)). In some embodiments, a therapeutically effective amount of a composition can result in an increase in the expression level of an active target protein (e.g., an active supporting cell target protein (e.g., a wildtype, full-length supporting cell target protein or active variant) ort an active hair cell target protein (e.g., a wildtype, full-length hair cell target protein or active variant) in a target cell (e.g., a supporting cell or a hair cell). In some embodiments, a therapeutically effective amount of a composition can result in an increase in the expression level of an active target protein (e.g., an active supporting cell target protein (e.g., a wildtype, full-length supporting cell target protein or active variant) or an active hair cell target protein (e.g., a wildtype, full-length hair cell target protein or active variant), and/or an increase in one or more activities of a target protein (e.g., a supporting cell target protein or a hair cell target protein) in a target cell (e.g., as compared to a reference level, such as the level(s) in a primate prior to treatment, the level(s) in a primate having a mutation in a target gene (e.g., a supporting cell target gene or a hair cell target gene), or the level(s) in a primate or a population of primates having hearing loss, e.g., non-syndromic sensorineural hearing loss or syndromic sensorineural hearing loss).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “active hair cell target protein” means a protein encoded by DNA that, if substituted for both wildtype alleles encoding full-length hair cell target protein in hair cells of what is otherwise a wildtype primate, and if expressed in the hair cells of that primate, results in that primate's having a level of hearing approximating the normal level of hearing of a similar primate that is entirely wildtype. Non-limiting examples of active hair cell target proteins are full-length hair cell target proteins (e.g., any of the full-length hair cell target proteins described herein).

The term “active supporting cell target protein” means a protein encoded by DNA that, if substituted for both wildtype alleles encoding full-length supporting cell target protein in supporting cells of what is otherwise a wildtype primate, and if expressed in the supporting cells of that primate, results in that primate's having a level of hearing approximating the normal level of hearing of a similar primate that is entirely wildtype. Non-limiting examples of active supporting cell target proteins are full-length supporting cell target proteins (e.g., any of the full-length supporting cell target proteins described herein).

For example, an active target protein (e.g., an active supporting cell target protein or an active hair cell target protein) can include a sequence of a wildtype, full-length target protein (e.g., a supporting cell target protein (e.g., a wildtype, human, full-length supporting cell target protein) or a hair cell target protein (e.g., a wildtype, human, full-length hair cell target protein) including 1 amino acid substitution to about 160 amino acid substitutions, 1 amino acid substitution to about 155 amino acid substitutions, 1 amino acid substitution to about 150 amino acid substitutions, 1 amino acid substitution to about 145 amino acid substitutions, 1 amino acid substitution to about 140 amino acid substitutions, 1 amino acid substitution to about 135 amino acid substitutions, 1 amino acid substitution to about 130 amino acid substitutions, 1 amino acid substitution to about 125 amino acid substitutions, 1 amino acid substitution to about 120 amino acid substitutions, 1 amino acid substitution to about 115 amino acid substitutions, 1 amino acid substitution to about 110 amino acid substitutions, 1 amino acid substitution to about 105 amino acid substitutions, 1 amino acid substitution to about 100 amino acid substitutions, 1 amino acid substitution to about 95 amino acid substitutions, 1 amino acid substitution to about 90 amino acid substitutions, 1 amino acid substitution to about 85 amino acid substitutions, 1 amino acid substitution to about 80 amino acid substitutions, 1 amino acid substitution to about 75 amino acid substitutions, 1 amino acid substitution to about 70 amino acid substitutions, 1 amino acid substitution to about 65 amino acid substitutions, 1 amino acid substitution to about 60 amino acid substitutions, 1 amino acid substitution to about 55 amino acid substitutions, 1 amino acid substitution to about 50 amino acid substitutions, 1 amino acid substitution to about 45 amino acid substitutions, 1 amino acid substitution to about 40 amino acid substitutions, 1 amino acid substitution to about 35 amino acid substitutions, 1 amino acid substitution to about 30 amino acid substitutions, 1 amino acid substitution to about 25 amino acid substitutions, 1 amino acid substitution to about 20 amino acid substitutions, 1 amino acid substitution to about 15 amino acid substitutions, 1 amino acid substitution to about 10 amino acid substitutions, 1 amino acid substitution to about 9 amino acid substitutions, 1 amino acid substitution to about 8 amino acid substitutions, 1 amino acid substitution to about 7 amino acid substitutions, 1 amino acid substitution to about 6 amino acid substitutions, 1 amino acid substitution to about 5 amino acid substitutions, 1 amino acid substitution to about 4 amino acid substitutions, 1 amino acid substitution to about 3 amino acid substitutions, between about 2 amino acid substitutions to about 160 amino acid substitutions, about 2 amino acid substitutions to about 155 amino acid substitutions, about 2 amino acid substitutions to about 150 amino acid substitutions, about 2 amino acid substitutions to about 145 amino acid substitutions, about 2 amino acid substitutions to about 140 amino acid substitutions, about 2 amino acid substitutions to about 135 amino acid substitutions, about 2 amino acid substitutions to about 130 amino acid substitutions, about 2 amino acid substitutions to about 125 amino acid substitutions, about 2 amino acid substitutions to about 120 amino acid substitutions, about 2 amino acid substitutions to about 115 amino acid substitutions, about 2 amino acid substitutions to about 110 amino acid substitutions, about 2 amino acid substitutions to about 105 amino acid substitutions, about 2 amino acid substitutions to about 100 amino acid substitutions, about 2 amino acid substitutions to about 95 amino acid substitutions, about 2 amino acid substitutions to about 90 amino acid substitutions, about 2 amino acid substitutions to about 85 amino acid substitutions, about 2 amino acid substitutions to about 80 amino acid substitutions, about 2 amino acid substitutions to about 75 amino acid substitutions, about 2 amino acid substitutions to about 70 amino acid substitutions, about 2 amino acid substitutions to about 65 amino acid substitutions, about 2 amino acid substitutions to about 60 amino acid substitutions, about 2 amino acid substitutions to about 55 amino acid substitutions, about 2 amino acid substitutions to about 50 amino acid substitutions, about 2 amino acid substitutions to about 45 amino acid substitutions, about 2 amino acid substitutions to about 40 amino acid substitutions, about 2 amino acid substitutions to about 35 amino acid substitutions, about 2 amino acid substitutions to about 30 amino acid substitutions, about 2 amino acid substitutions to about 25 amino acid substitutions, about 2 amino acid substitutions to about 20 amino acid substitutions, about 2 amino acid substitutions to about 15 amino acid substitutions, about 2 amino acid substitutions to about 10 amino acid substitutions, about 2 amino acid substitutions to about 9 amino acid substitutions, about 2 amino acid substitutions to about 8 amino acid substitutions, about 2 amino acid substitutions to about 7 amino acid substitutions, about 2 amino acid substitutions to about 6 amino acid substitutions, about 2 amino acid substitutions to about 5 amino acid substitutions, about 2 amino acid substitutions to about 4 amino acid substitutions, between about 3 amino acid substitutions to about 160 amino acid substitutions, about 3 amino acid substitutions to about 155 amino acid substitutions, about 3 amino acid substitutions to about 150 amino acid substitutions, about 3 amino acid substitutions to about 145 amino acid substitutions, about 3 amino acid substitutions to about 140 amino acid substitutions, about 3 amino acid substitutions to about 135 amino acid substitutions, about 3 amino acid substitutions to about 130 amino acid substitutions, about 3 amino acid substitutions to about 125 amino acid substitutions, about 3 amino acid substitutions to about 120 amino acid substitutions, about 3 amino acid substitutions to about 115 amino acid substitutions, about 3 amino acid substitutions to about 110 amino acid substitutions, about 3 amino acid substitutions to about 105 amino acid substitutions, about 3 amino acid substitutions to about 100 amino acid substitutions, about 3 amino acid substitutions to about 95 amino acid substitutions, about 3 amino acid substitutions to about 90 amino acid substitutions, about 3 amino acid substitutions to about 85 amino acid substitutions, about 3 amino acid substitutions to about 80 amino acid substitutions, about 3 amino acid substitutions to about 75 amino acid substitutions, about 3 amino acid substitutions to about 70 amino acid substitutions, about 3 amino acid substitutions to about 65 amino acid substitutions, about 3 amino acid substitutions to about 60 amino acid substitutions, about 3 amino acid substitutions to about 55 amino acid substitutions, about 3 amino acid substitutions to about 50 amino acid substitutions, about 3 amino acid substitutions to about 45 amino acid substitutions, about 3 amino acid substitutions to about 40 amino acid substitutions, about 3 amino acid substitutions to about 35 amino acid substitutions, about 3 amino acid substitutions to about 30 amino acid substitutions, about 3 amino acid substitutions to about 25 amino acid substitutions, about 3 amino acid substitutions to about 20 amino acid substitutions, about 3 amino acid substitutions to about 15 amino acid substitutions, about 3 amino acid substitutions to about 10 amino acid substitutions, about 3 amino acid substitutions to about 9 amino acid substitutions, about 3 amino acid substitutions to about 8 amino acid substitutions, about 3 amino acid substitutions to about 7 amino acid substitutions, about 3 amino acid substitutions to about 6 amino acid substitutions, about 3 amino acid substitutions to about 5 amino acid substitutions, between about 4 amino acid substitutions to about 160 amino acid substitutions, about 4 amino acid substitutions to about 155 amino acid substitutions, about 4 amino acid substitutions to about 150 amino acid substitutions, about 4 amino acid substitutions to about 145 amino acid substitutions, about 4 amino acid substitutions to about 140 amino acid substitutions, about 4 amino acid substitutions to about 135 amino acid substitutions, about 4 amino acid substitutions to about 130 amino acid substitutions, about 4 amino acid substitutions to about 125 amino acid substitutions, about 4 amino acid substitutions to about 120 amino acid substitutions, about 4 amino acid substitutions to about 115 amino acid substitutions, about 4 amino acid substitutions to about 110 amino acid substitutions, about 4 amino acid substitutions to about 105 amino acid substitutions, about 4 amino acid substitutions to about 100 amino acid substitutions, about 4 amino acid substitutions to about 95 amino acid substitutions, about 4 amino acid substitutions to about 90 amino acid substitutions, about 4 amino acid substitutions to about 85 amino acid substitutions, about 4 amino acid substitutions to about 80 amino acid substitutions, about 4 amino acid substitutions to about 75 amino acid substitutions, about 4 amino acid substitutions to about 70 amino acid substitutions, about 4 amino acid substitutions to about 65 amino acid substitutions, about 4 amino acid substitutions to about 60 amino acid substitutions, about 4 amino acid substitutions to about 55 amino acid substitutions, about 4 amino acid substitutions to about 50 amino acid substitutions, about 4 amino acid substitutions to about 45 amino acid substitutions, about 4 amino acid substitutions to about 40 amino acid substitutions, about 4 amino acid substitutions to about 35 amino acid substitutions, about 4 amino acid substitutions to about 30 amino acid substitutions, about 4 amino acid substitutions to about 25 amino acid substitutions, about 4 amino acid substitutions to about 20 amino acid substitutions, about 4 amino acid substitutions to about 15 amino acid substitutions, about 4 amino acid substitutions to about 10 amino acid substitutions, about 4 amino acid substitutions to about 9 amino acid substitutions, about 4 amino acid substitutions to about 8 amino acid substitutions, about 4 amino acid substitutions to about 7 amino acid substitutions, about 4 amino acid substitutions to about 6 amino acid substitutions, between about 5 amino acid substitutions to about 160 amino acid substitutions, about 5 amino acid substitutions to about 155 amino acid substitutions, about 5 amino acid substitutions to about 150 amino acid substitutions, about 5 amino acid substitutions to about 145 amino acid substitutions, about 5 amino acid substitutions to about 140 amino acid substitutions, about 5 amino acid substitutions to about 135 amino acid substitutions, about 5 amino acid substitutions to about 130 amino acid substitutions, about 5 amino acid substitutions to about 125 amino acid substitutions, about 5 amino acid substitutions to about 120 amino acid substitutions, about 5 amino acid substitutions to about 115 amino acid substitutions, about 5 amino acid substitutions to about 110 amino acid substitutions, about 5 amino acid substitutions to about 105 amino acid substitutions, about 5 amino acid substitutions to about 100 amino acid substitutions, about 5 amino acid substitutions to about 95 amino acid substitutions, about 5 amino acid substitutions to about 90 amino acid substitutions, about 5 amino acid substitutions to about 85 amino acid substitutions, about 5 amino acid substitutions to about 80 amino acid substitutions, about 5 amino acid substitutions to about 75 amino acid substitutions, about 5 amino acid substitutions to about 70 amino acid substitutions, about 5 amino acid substitutions to about 65 amino acid substitutions, about 5 amino acid substitutions to about 60 amino acid substitutions, about 5 amino acid substitutions to about 55 amino acid substitutions, about 5 amino acid substitutions to about 50 amino acid substitutions, about 5 amino acid substitutions to about 45 amino acid substitutions, about 5 amino acid substitutions to about 40 amino acid substitutions, about 5 amino acid substitutions to about 35 amino acid substitutions, about 5 amino acid substitutions to about 30 amino acid substitutions, about 5 amino acid substitutions to about 25 amino acid substitutions, about 5 amino acid substitutions to about 20 amino acid substitutions, about 5 amino acid substitutions to about 15 amino acid substitutions, about 5 amino acid substitutions to about 10 amino acid substitutions, about 5 amino acid substitutions to about 9 amino acid substitutions, about 5 amino acid substitutions to about 8 amino acid substitutions, about 5 amino acid substitutions to about 7 amino acid substitutions, between about 6 amino acid substitutions to about 160 amino acid substitutions, about 6 amino acid substitutions to about 155 amino acid substitutions, about 6 amino acid substitutions to about 150 amino acid substitutions, about 6 amino acid substitutions to about 145 amino acid substitutions, about 6 amino acid substitutions to about 140 amino acid substitutions, about 6 amino acid substitutions to about 135 amino acid substitutions, about 6 amino acid substitutions to about 130 amino acid substitutions, about 6 amino acid substitutions to about 125 amino acid substitutions, about 6 amino acid substitutions to about 120 amino acid substitutions, about 6 amino acid substitutions to about 115 amino acid substitutions, about 6 amino acid substitutions to about 110 amino acid substitutions, about 6 amino acid substitutions to about 105 amino acid substitutions, about 6 amino acid substitutions to about 100 amino acid substitutions, about 6 amino acid substitutions to about 95 amino acid substitutions, about 6 amino acid substitutions to about 90 amino acid substitutions, about 6 amino acid substitutions to about 85 amino acid substitutions, about 6 amino acid substitutions to about 80 amino acid substitutions, about 6 amino acid substitutions to about 75 amino acid substitutions, about 6 amino acid substitutions to about 70 amino acid substitutions, about 6 amino acid substitutions to about 65 amino acid substitutions, about 6 amino acid substitutions to about 60 amino acid substitutions, about 6 amino acid substitutions to about 55 amino acid substitutions, about 6 amino acid substitutions to about 50 amino acid substitutions, about 6 amino acid substitutions to about 45 amino acid substitutions, about 6 amino acid substitutions to about 40 amino acid substitutions, about 6 amino acid substitutions to about 35 amino acid substitutions, about 6 amino acid substitutions to about 30 amino acid substitutions, about 6 amino acid substitutions to about 25 amino acid substitutions, about 6 amino acid substitutions to about 20 amino acid substitutions, about 6 amino acid substitutions to about 15 amino acid substitutions, about 6 amino acid substitutions to about 10 amino acid substitutions, about 6 amino acid substitutions to about 9 amino acid substitutions, about 6 amino acid substitutions to about 8 amino acid substitutions, between about 7 amino acid substitutions to about 160 amino acid substitutions, about 7 amino acid substitutions to about 155 amino acid substitutions, about 7 amino acid substitutions to about 150 amino acid substitutions, about 7 amino acid substitutions to about 145 amino acid substitutions, about 7 amino acid substitutions to about 140 amino acid substitutions, about 7 amino acid substitutions to about 135 amino acid substitutions, about 7 amino acid substitutions to about 130 amino acid substitutions, about 7 amino acid substitutions to about 125 amino acid substitutions, about 7 amino acid substitutions to about 120 amino acid substitutions, about 7 amino acid substitutions to about 115 amino acid substitutions, about 7 amino acid substitutions to about 110 amino acid substitutions, about 7 amino acid substitutions to about 105 amino acid substitutions, about 7 amino acid substitutions to about 100 amino acid substitutions, about 7 amino acid substitutions to about 95 amino acid substitutions, about 7 amino acid substitutions to about 90 amino acid substitutions, about 7 amino acid substitutions to about 85 amino acid substitutions, about 7 amino acid substitutions to about 80 amino acid substitutions, about 7 amino acid substitutions to about 75 amino acid substitutions, about 7 amino acid substitutions to about 70 amino acid substitutions, about 7 amino acid substitutions to about 65 amino acid substitutions, about 7 amino acid substitutions to about 60 amino acid substitutions, about 7 amino acid substitutions to about 55 amino acid substitutions, about 7 amino acid substitutions to about 50 amino acid substitutions, about 7 amino acid substitutions to about 45 amino acid substitutions, about 7 amino acid substitutions to about 40 amino acid substitutions, about 7 amino acid substitutions to about 35 amino acid substitutions, about 7 amino acid substitutions to about 30 amino acid substitutions, about 7 amino acid substitutions to about 25 amino acid substitutions, about 7 amino acid substitutions to about 20 amino acid substitutions, about 7 amino acid substitutions to about 15 amino acid substitutions, about 7 amino acid substitutions to about 10 amino acid substitutions, about 7 amino acid substitutions to about 9 amino acid substitutions, between about 8 amino acid substitutions to about 160 amino acid substitutions, about 8 amino acid substitutions to about 155 amino acid substitutions, about 8 amino acid substitutions to about 150 amino acid substitutions, about 8 amino acid substitutions to about 145 amino acid substitutions, about 8 amino acid substitutions to about 140 amino acid substitutions, about 8 amino acid substitutions to about 135 amino acid substitutions, about 8 amino acid substitutions to about 130 amino acid substitutions, about 8 amino acid substitutions to about 125 amino acid substitutions, about 8 amino acid substitutions to about 120 amino acid substitutions, about 8 amino acid substitutions to about 115 amino acid substitutions, about 8 amino acid substitutions to about 110 amino acid substitutions, about 8 amino acid substitutions to about 105 amino acid substitutions, about 8 amino acid substitutions to about 100 amino acid substitutions, about 8 amino acid substitutions to about 95 amino acid substitutions, about 8 amino acid substitutions to about 90 amino acid substitutions, about 8 amino acid substitutions to about 85 amino acid substitutions, about 8 amino acid substitutions to about 80 amino acid substitutions, about 8 amino acid substitutions to about 75 amino acid substitutions, about 8 amino acid substitutions to about 70 amino acid substitutions, about 8 amino acid substitutions to about 65 amino acid substitutions, about 8 amino acid substitutions to about 60 amino acid substitutions, about 8 amino acid substitutions to about 55 amino acid substitutions, about 8 amino acid substitutions to about 50 amino acid substitutions, about 8 amino acid substitutions to about 45 amino acid substitutions, about 8 amino acid substitutions to about 40 amino acid substitutions, about 8 amino acid substitutions to about 35 amino acid substitutions, about 8 amino acid substitutions to about 30 amino acid substitutions, about 8 amino acid substitutions to about 25 amino acid substitutions, about 8 amino acid substitutions to about 20 amino acid substitutions, about 8 amino acid substitutions to about 15 amino acid substitutions, about 8 amino acid substitutions to about 10 amino acid substitutions, between about 10 amino acid substitutions to about 160 amino acid substitutions, about 10 amino acid substitutions to about 155 amino acid substitutions, about 10 amino acid substitutions to about 150 amino acid substitutions, about 10 amino acid substitutions to about 145 amino acid substitutions, about 10 amino acid substitutions to about 140 amino acid substitutions, about 10 amino acid substitutions to about 135 amino acid substitutions, about 10 amino acid substitutions to about 130 amino acid substitutions, about 10 amino acid substitutions to about 125 amino acid substitutions, about 10 amino acid substitutions to about 120 amino acid substitutions, about 10 amino acid substitutions to about 115 amino acid substitutions, about 10 amino acid substitutions to about 110 amino acid substitutions, about 10 amino acid substitutions to about 105 amino acid substitutions, about 10 amino acid substitutions to about 100 amino acid substitutions, about 10 amino acid substitutions to about 95 amino acid substitutions, about 10 amino acid substitutions to about 90 amino acid substitutions, about 10 amino acid substitutions to about 85 amino acid substitutions, about 10 amino acid substitutions to about 80 amino acid substitutions, about 10 amino acid substitutions to about 75 amino acid substitutions, about 10 amino acid substitutions to about 70 amino acid substitutions, about 10 amino acid substitutions to about 65 amino acid substitutions, about 10 amino acid substitutions to about 60 amino acid substitutions, about 10 amino acid substitutions to about 55 amino acid substitutions, about 10 amino acid substitutions to about 50 amino acid substitutions, about 10 amino acid substitutions to about 45 amino acid substitutions, about 10 amino acid substitutions to about 40 amino acid substitutions, about 10 amino acid substitutions to about 35 amino acid substitutions, about 10 amino acid substitutions to about 30 amino acid substitutions, about 10 amino acid substitutions to about 25 amino acid substitutions, about 10 amino acid substitutions to about 20 amino acid substitutions, about 10 amino acid substitutions to about 15 amino acid substitutions, between about 15 amino acid substitutions to about 160 amino acid substitutions, about 15 amino acid substitutions to about 155 amino acid substitutions, about 15 amino acid substitutions to about 150 amino acid substitutions, about 15 amino acid substitutions to about 145 amino acid substitutions, about 15 amino acid substitutions to about 140 amino acid substitutions, about 15 amino acid substitutions to about 135 amino acid substitutions, about 15 amino acid substitutions to about 130 amino acid substitutions, about 15 amino acid substitutions to about 125 amino acid substitutions, about 15 amino acid substitutions to about 120 amino acid substitutions, about 15 amino acid substitutions to about 115 amino acid substitutions, about 15 amino acid substitutions to about 110 amino acid substitutions, about 15 amino acid substitutions to about 105 amino acid substitutions, about 15 amino acid substitutions to about 100 amino acid substitutions, about 15 amino acid substitutions to about 95 amino acid substitutions, about 15 amino acid substitutions to about 90 amino acid substitutions, about 15 amino acid substitutions to about 85 amino acid substitutions, about 15 amino acid substitutions to about 80 amino acid substitutions, about 15 amino acid substitutions to about 75 amino acid substitutions, about 15 amino acid substitutions to about 70 amino acid substitutions, about 15 amino acid substitutions to about 65 amino acid substitutions, about 15 amino acid substitutions to about 60 amino acid substitutions, about 15 amino acid substitutions to about 55 amino acid substitutions, about 15 amino acid substitutions to about 50 amino acid substitutions, about 15 amino acid substitutions to about 45 amino acid substitutions, about 15 amino acid substitutions to about 40 amino acid substitutions, about 15 amino acid substitutions to about 35 amino acid substitutions, about 15 amino acid substitutions to about 30 amino acid substitutions, about 15 amino acid substitutions to about 25 amino acid substitutions, about 15 amino acid substitutions to about 20 amino acid substitutions, between about 20 amino acid substitutions to about 160 amino acid substitutions, about 20 amino acid substitutions to about 155 amino acid substitutions, about 20 amino acid substitutions to about 150 amino acid substitutions, about 20 amino acid substitutions to about 145 amino acid substitutions, about 20 amino acid substitutions to about 140 amino acid substitutions, about 20 amino acid substitutions to about 135 amino acid substitutions, about 20 amino acid substitutions to about 130 amino acid substitutions, about 20 amino acid substitutions to about 125 amino acid substitutions, about 20 amino acid substitutions to about 120 amino acid substitutions, about 20 amino acid substitutions to about 115 amino acid substitutions, about 20 amino acid substitutions to about 110 amino acid substitutions, about 20 amino acid substitutions to about 105 amino acid substitutions, about 20 amino acid substitutions to about 100 amino acid substitutions, about 20 amino acid substitutions to about 95 amino acid substitutions, about 20 amino acid substitutions to about 90 amino acid substitutions, about 20 amino acid substitutions to about 85 amino acid substitutions, about 20 amino acid substitutions to about 80 amino acid substitutions, about 20 amino acid substitutions to about 75 amino acid substitutions, about 20 amino acid substitutions to about 70 amino acid substitutions, about 20 amino acid substitutions to about 65 amino acid substitutions, about 20 amino acid substitutions to about 60 amino acid substitutions, about 20 amino acid substitutions to about 55 amino acid substitutions, about 20 amino acid substitutions to about 50 amino acid substitutions, about 20 amino acid substitutions to about 45 amino acid substitutions, about 20 amino acid substitutions to about 40 amino acid substitutions, about 20 amino acid substitutions to about 35 amino acid substitutions, about 20 amino acid substitutions to about 30 amino acid substitutions, about 20 amino acid substitutions to about 25 amino acid substitutions, between about 25 amino acid substitutions to about 160 amino acid substitutions, about 25 amino acid substitutions to about 155 amino acid substitutions, about 25 amino acid substitutions to about 150 amino acid substitutions, about 25 amino acid substitutions to about 145 amino acid substitutions, about 25 amino acid substitutions to about 140 amino acid substitutions, about 25 amino acid substitutions to about 135 amino acid substitutions, about 25 amino acid substitutions to about 130 amino acid substitutions, about 25 amino acid substitutions to about 125 amino acid substitutions, about 25 amino acid substitutions to about 120 amino acid substitutions, about 25 amino acid substitutions to about 115 amino acid substitutions, about 25 amino acid substitutions to about 110 amino acid substitutions, about 25 amino acid substitutions to about 105 amino acid substitutions, about 25 amino acid substitutions to about 100 amino acid substitutions, about 25 amino acid substitutions to about 95 amino acid substitutions, about 25 amino acid substitutions to about 90 amino acid substitutions, about 25 amino acid substitutions to about 85 amino acid substitutions, about 25 amino acid substitutions to about 80 amino acid substitutions, about 25 amino acid substitutions to about 75 amino acid substitutions, about 25 amino acid substitutions to about 70 amino acid substitutions, about 25 amino acid substitutions to about 65 amino acid substitutions, about 25 amino acid substitutions to about 60 amino acid substitutions, about 25 amino acid substitutions to about 55 amino acid substitutions, about 25 amino acid substitutions to about 50 amino acid substitutions, about 25 amino acid substitutions to about 45 amino acid substitutions, about 25 amino acid substitutions to about 40 amino acid substitutions, about 25 amino acid substitutions to about 35 amino acid substitutions, about 25 amino acid substitutions to about 30 amino acid substitutions, between about 30 amino acid substitutions to about 160 amino acid substitutions, about 30 amino acid substitutions to about 155 amino acid substitutions, about 30 amino acid substitutions to about 150 amino acid substitutions, about 30 amino acid substitutions to about 145 amino acid substitutions, about 30 amino acid substitutions to about 140 amino acid substitutions, about 30 amino acid substitutions to about 135 amino acid substitutions, about 30 amino acid substitutions to about 130 amino acid substitutions, about 30 amino acid substitutions to about 125 amino acid substitutions, about 30 amino acid substitutions to about 120 amino acid substitutions, about 30 amino acid substitutions to about 115 amino acid substitutions, about 30 amino acid substitutions to about 110 amino acid substitutions, about 30 amino acid substitutions to about 105 amino acid substitutions, about 30 amino acid substitutions to about 100 amino acid substitutions, about 30 amino acid substitutions to about 95 amino acid substitutions, about 30 amino acid substitutions to about 90 amino acid substitutions, about 30 amino acid substitutions to about 85 amino acid substitutions, about 30 amino acid substitutions to about 80 amino acid substitutions, about 30 amino acid substitutions to about 75 amino acid substitutions, about 30 amino acid substitutions to about 70 amino acid substitutions, about 30 amino acid substitutions to about 65 amino acid substitutions, about 30 amino acid substitutions to about 60 amino acid substitutions, about 30 amino acid substitutions to about 55 amino acid substitutions, about 30 amino acid substitutions to about 50 amino acid substitutions, about 30 amino acid substitutions to about 45 amino acid substitutions, about 30 amino acid substitutions to about 40 amino acid substitutions, about 30 amino acid substitutions to about 35 amino acid substitutions, between about 35 amino acid substitutions to about 160 amino acid substitutions, about 35 amino acid substitutions to about 155 amino acid substitutions, about 35 amino acid substitutions to about 150 amino acid substitutions, about 35 amino acid substitutions to about 145 amino acid substitutions, about 35 amino acid substitutions to about 140 amino acid substitutions, about 35 amino acid substitutions to about 135 amino acid substitutions, about 35 amino acid substitutions to about 130 amino acid substitutions, about 35 amino acid substitutions to about 125 amino acid substitutions, about 35 amino acid substitutions to about 120 amino acid substitutions, about 35 amino acid substitutions to about 115 amino acid substitutions, about 35 amino acid substitutions to about 110 amino acid substitutions, about 35 amino acid substitutions to about 105 amino acid substitutions, about 35 amino acid substitutions to about 100 amino acid substitutions, about 35 amino acid substitutions to about 95 amino acid substitutions, about 35 amino acid substitutions to about 90 amino acid substitutions, about 35 amino acid substitutions to about 85 amino acid substitutions, about 35 amino acid substitutions to about 80 amino acid substitutions, about 35 amino acid substitutions to about 75 amino acid substitutions, about 35 amino acid substitutions to about 70 amino acid substitutions, about 35 amino acid substitutions to about 65 amino acid substitutions, about 35 amino acid substitutions to about 60 amino acid substitutions, about 35 amino acid substitutions to about 55 amino acid substitutions, about 35 amino acid substitutions to about 50 amino acid substitutions, about 35 amino acid substitutions to about 45 amino acid substitutions, about 35 amino acid substitutions to about 40 amino acid substitutions, between about 40 amino acid substitutions to about 160 amino acid substitutions, about 40 amino acid substitutions to about 155 amino acid substitutions, about 40 amino acid substitutions to about 150 amino acid substitutions, about 40 amino acid substitutions to about 145 amino acid substitutions, about 40 amino acid substitutions to about 140 amino acid substitutions, about 40 amino acid substitutions to about 135 amino acid substitutions, about 40 amino acid substitutions to about 130 amino acid substitutions, about 40 amino acid substitutions to about 125 amino acid substitutions, about 40 amino acid substitutions to about 120 amino acid substitutions, about 40 amino acid substitutions to about 115 amino acid substitutions, about 40 amino acid substitutions to about 110 amino acid substitutions, about 40 amino acid substitutions to about 105 amino acid substitutions, about 40 amino acid substitutions to about 100 amino acid substitutions, about 40 amino acid substitutions to about 95 amino acid substitutions, about 40 amino acid substitutions to about 90 amino acid substitutions, about 40 amino acid substitutions to about 85 amino acid substitutions, about 40 amino acid substitutions to about 80 amino acid substitutions, about 40 amino acid substitutions to about 75 amino acid substitutions, about 40 amino acid substitutions to about 70 amino acid substitutions, about 40 amino acid substitutions to about 65 amino acid substitutions, about 40 amino acid substitutions to about 60 amino acid substitutions, about 40 amino acid substitutions to about 55 amino acid substitutions, about 40 amino acid substitutions to about 50 amino acid substitutions, about 40 amino acid substitutions to about 45 amino acid substitutions, between about 45 amino acid substitutions to about 160 amino acid substitutions, about 45 amino acid substitutions to about 155 amino acid substitutions, about 45 amino acid substitutions to about 150 amino acid substitutions, about 45 amino acid substitutions to about 145 amino acid substitutions, about 45 amino acid substitutions to about 140 amino acid substitutions, about 45 amino acid substitutions to about 135 amino acid substitutions, about 45 amino acid substitutions to about 130 amino acid substitutions, about 45 amino acid substitutions to about 125 amino acid substitutions, about 45 amino acid substitutions to about 120 amino acid substitutions, about 45 amino acid substitutions to about 115 amino acid substitutions, about 45 amino acid substitutions to about 110 amino acid substitutions, about 45 amino acid substitutions to about 105 amino acid substitutions, about 45 amino acid substitutions to about 100 amino acid substitutions, about 45 amino acid substitutions to about 95 amino acid substitutions, about 45 amino acid substitutions to about 90 amino acid substitutions, about 45 amino acid substitutions to about 85 amino acid substitutions, about 45 amino acid substitutions to about 80 amino acid substitutions, about 45 amino acid substitutions to about 75 amino acid substitutions, about 45 amino acid substitutions to about 70 amino acid substitutions, about 45 amino acid substitutions to about 65 amino acid substitutions, about 45 amino acid substitutions to about 60 amino acid substitutions, about 45 amino acid substitutions to about 55 amino acid substitutions, about 45 amino acid substitutions to about 50 amino acid substitutions, between about 50 amino acid substitutions to about 160 amino acid substitutions, about 50 amino acid substitutions to about 155 amino acid substitutions, about 50 amino acid substitutions to about 150 amino acid substitutions, about 50 amino acid substitutions to about 145 amino acid substitutions, about 50 amino acid substitutions to about 140 amino acid substitutions, about 50 amino acid substitutions to about 135 amino acid substitutions, about 50 amino acid substitutions to about 130 amino acid substitutions, about 50 amino acid substitutions to about 125 amino acid substitutions, about 50 amino acid substitutions to about 120 amino acid substitutions, about 50 amino acid substitutions to about 115 amino acid substitutions, about 50 amino acid substitutions to about 110 amino acid substitutions, about 50 amino acid substitutions to about 105 amino acid substitutions, about 50 amino acid substitutions to about 100 amino acid substitutions, about 50 amino acid substitutions to about 95 amino acid substitutions, about 50 amino acid substitutions to about 90 amino acid substitutions, about 50 amino acid substitutions to about 85 amino acid substitutions, about 50 amino acid substitutions to about 80 amino acid substitutions, about 50 amino acid substitutions to about 75 amino acid substitutions, about 50 amino acid substitutions to about 70 amino acid substitutions, about 50 amino acid substitutions to about 65 amino acid substitutions, about 50 amino acid substitutions to about 60 amino acid substitutions, about 50 amino acid substitutions to about 55 amino acid substitutions, between about 60 amino acid substitutions to about 160 amino acid substitutions, about 60 amino acid substitutions to about 155 amino acid substitutions, about 60 amino acid substitutions to about 150 amino acid substitutions, about 60 amino acid substitutions to about 145 amino acid substitutions, about 60 amino acid substitutions to about 140 amino acid substitutions, about 60 amino acid substitutions to about 135 amino acid substitutions, about 60 amino acid substitutions to about 130 amino acid substitutions, about 60 amino acid substitutions to about 125 amino acid substitutions, about 60 amino acid substitutions to about 120 amino acid substitutions, about 60 amino acid substitutions to about 115 amino acid substitutions, about 60 amino acid substitutions to about 110 amino acid substitutions, about 60 amino acid substitutions to about 105 amino acid substitutions, about 60 amino acid substitutions to about 100 amino acid substitutions, about 60 amino acid substitutions to about 95 amino acid substitutions, about 60 amino acid substitutions to about 90 amino acid substitutions, about 60 amino acid substitutions to about 85 amino acid substitutions, about 60 amino acid substitutions to about 80 amino acid substitutions, about 60 amino acid substitutions to about 75 amino acid substitutions, about 60 amino acid substitutions to about 70 amino acid substitutions, about 60 amino acid substitutions to about 65 amino acid substitutions, between about 70 amino acid substitutions to about 160 amino acid substitutions, about 70 amino acid substitutions to about 155 amino acid substitutions, about 70 amino acid substitutions to about 150 amino acid substitutions, about 70 amino acid substitutions to about 145 amino acid substitutions, about 70 amino acid substitutions to about 140 amino acid substitutions, about 70 amino acid substitutions to about 135 amino acid substitutions, about 70 amino acid substitutions to about 130 amino acid substitutions, about 70 amino acid substitutions to about 125 amino acid substitutions, about 70 amino acid substitutions to about 120 amino acid substitutions, about 70 amino acid substitutions to about 115 amino acid substitutions, about 70 amino acid substitutions to about 110 amino acid substitutions, about 70 amino acid substitutions to about 105 amino acid substitutions, about 70 amino acid substitutions to about 100 amino acid substitutions, about 70 amino acid substitutions to about 95 amino acid substitutions, about 70 amino acid substitutions to about 90 amino acid substitutions, about 70 amino acid substitutions to about 85 amino acid substitutions, about 70 amino acid substitutions to about 80 amino acid substitutions, about 70 amino acid substitutions to about 75 amino acid substitutions, between about 80 amino acid substitutions to about 160 amino acid substitutions, about 80 amino acid substitutions to about 155 amino acid substitutions, about 80 amino acid substitutions to about 150 amino acid substitutions, about 80 amino acid substitutions to about 145 amino acid substitutions, about 80 amino acid substitutions to about 140 amino acid substitutions, about 80 amino acid substitutions to about 135 amino acid substitutions, about 80 amino acid substitutions to about 130 amino acid substitutions, about 80 amino acid substitutions to about 125 amino acid substitutions, about 80 amino acid substitutions to about 120 amino acid substitutions, about 80 amino acid substitutions to about 115 amino acid substitutions, about 80 amino acid substitutions to about 110 amino acid substitutions, about 80 amino acid substitutions to about 105 amino acid substitutions, about 80 amino acid substitutions to about 100 amino acid substitutions, about 80 amino acid substitutions to about 95 amino acid substitutions, about 80 amino acid substitutions to about 90 amino acid substitutions, about 80 amino acid substitutions to about 85 amino acid substitutions, between about 90 amino acid substitutions to about 160 amino acid substitutions, about 90 amino acid substitutions to about 155 amino acid substitutions, about 90 amino acid substitutions to about 150 amino acid substitutions, about 90 amino acid substitutions to about 145 amino acid substitutions, about 90 amino acid substitutions to about 140 amino acid substitutions, about 90 amino acid substitutions to about 135 amino acid substitutions, about 90 amino acid substitutions to about 130 amino acid substitutions, about 90 amino acid substitutions to about 125 amino acid substitutions, about 90 amino acid substitutions to about 120 amino acid substitutions, about 90 amino acid substitutions to about 115 amino acid substitutions, about 90 amino acid substitutions to about 110 amino acid substitutions, about 90 amino acid substitutions to about 105 amino acid substitutions, about 90 amino acid substitutions to about 100 amino acid substitutions, about 90 amino acid substitutions to about 95 amino acid substitutions, between about 100 amino acid substitutions to about 160 amino acid substitutions, about 100 amino acid substitutions to about 155 amino acid substitutions, about 100 amino acid substitutions to about 150 amino acid substitutions, about 100 amino acid substitutions to about 145 amino acid substitutions, about 100 amino acid substitutions to about 140 amino acid substitutions, about 100 amino acid substitutions to about 135 amino acid substitutions, about 100 amino acid substitutions to about 130 amino acid substitutions, about 100 amino acid substitutions to about 125 amino acid substitutions, about 100 amino acid substitutions to about 120 amino acid substitutions, about 100 amino acid substitutions to about 115 amino acid substitutions, about 100 amino acid substitutions to about 110 amino acid substitutions, about 100 amino acid substitutions to about 105 amino acid substitutions, between about 110 amino acid substitutions to about 160 amino acid substitutions, about 110 amino acid substitutions to about 155 amino acid substitutions, about 110 amino acid substitutions to about 150 amino acid substitutions, about 110 amino acid substitutions to about 145 amino acid substitutions, about 110 amino acid substitutions to about 140 amino acid substitutions, about 110 amino acid substitutions to about 135 amino acid substitutions, about 110 amino acid substitutions to about 130 amino acid substitutions, about 110 amino acid substitutions to about 125 amino acid substitutions, about 110 amino acid substitutions to about 120 amino acid substitutions, about 110 amino acid substitutions to about 115 amino acid substitutions, between about 120 amino acid substitutions to about 160 amino acid substitutions, about 120 amino acid substitutions to about 155 amino acid substitutions, about 120 amino acid substitutions to about 150 amino acid substitutions, about 120 amino acid substitutions to about 145 amino acid substitutions, about 120 amino acid substitutions to about 140 amino acid substitutions, about 120 amino acid substitutions to about 135 amino acid substitutions, about 120 amino acid substitutions to about 130 amino acid substitutions, about 120 amino acid substitutions to about 125 amino acid substitutions, between about 130 amino acid substitutions to about 160 amino acid substitutions, about 130 amino acid substitutions to about 155 amino acid substitutions, about 130 amino acid substitutions to about 150 amino acid substitutions, about 130 amino acid substitutions to about 145 amino acid substitutions, about 130 amino acid substitutions to about 140 amino acid substitutions, about 130 amino acid substitutions to about 135 amino acid substitutions, between about 140 amino acid substitutions to about 160 amino acid substitutions, about 140 amino acid substitutions to about 155 amino acid substitutions, about 140 amino acid substitutions to about 150 amino acid substitutions, about 140 amino acid substitutions to about 145 amino acid substitutions, between about 150 amino acid substitutions to about 160 amino acid substitutions, or about 150 amino acid substitutions to about 155 amino acid substitutions. One skilled in the art would appreciate that amino acids that are conserved between wildtype supporting cell target proteins from different species or wildtype hair cell target proteins from different species can be mutated without losing activity, while those amino acids that are not conserved between wildtype supporting cell target proteins from different species or wildtype hair cell target proteins from different species should not be mutated as they are more likely (than amino acids that are not conserved between different species) to be involved in activity.

An active target protein (e.g., a supporting cell target protein or a hair cell target protein) can include, e.g., a sequence of a wildtype, full-length target protein (e.g., a supporting cell target protein (e.g., a wildtype, human, full-length supporting cell target protein) or a hair cell target protein (e.g., a wildtype, human, full-length hair cell target protein) that has 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 50 amino acids, about 40 amino acids to about 45 amino acids, about 45 amino acids to about 50 amino acids, deleted. In some embodiments where two or more amino acids are deleted from the sequence of a wildtype, full-length hair cell target protein, the two or more deleted amino acids can be contiguous in the sequence of the wildtype, full-length protein. In other examples where two or more amino acids are deleted from the sequence of a wildtype, full-length hair cell target protein, the two or more deleted amino acids are not contiguous in the sequence of the wildtype, full-length protein. One skilled in the art would appreciate that amino acids that are not conserved between wildtype, full-length hair cell target proteins from different species can be deleted without losing activity, while those amino acids that are conserved between wildtype, full-length hair cell target proteins from different species should not be deleted as they are more likely (than amino acids that are not conserved between different species) to be involved in activity.

In some embodiments where two or more amino acids are deleted from the sequence of a wildtype, full-length supporting cell target protein, the two or more deleted amino acids can be contiguous in the sequence of the wildtype, full-length protein. In other examples where two or more amino acids are deleted from the sequence of a wildtype, full-length supporting cell target protein, the two or more deleted amino acids are not contiguous in the sequence of the wildtype, full-length protein. One skilled in the art would appreciate that amino acids that are not conserved between wildtype, full-length supporting cell target proteins from different species can be deleted without losing activity, while those amino acids that are conserved between wildtype, full-length supporting cell target proteins from different species should not be deleted as they are more likely (than amino acids that are not conserved between different species) to be involved in activity.

In some examples, an active target protein (e.g., a supporting cell target protein or a hair cell target protein) can, e.g., include a sequence of a wildtype, full-length target protein (e.g., a wildtype, full-length supporting cell target protein or a wildtype, full-length hair cell target protein) that has between 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 100 amino acids, about 2 amino acid to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 100 amino acids, about 3 amino acid to about 95 amino acids, about 3 amino acids to about 90 amino acids, about 3 amino acids to about 85 amino acids, about 3 amino acids to about 80 amino acids, about 3 amino acids to about 75 amino acids, about 3 amino acids to about 70 amino acids, about 3 amino acids to about 65 amino acids, about 3 amino acids to about 60 amino acids, about 3 amino acids to about 55 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 100 amino acids, about 4 amino acid to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 100 amino acids, about 5 amino acid to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 100 amino acids, about 6 amino acid to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 100 amino acids, about 7 amino acid to about 95 amino acids, about 7 amino acids to about 90 amino acids, about 7 amino acids to about 85 amino acids, about 7 amino acids to about 80 amino acids, about 7 amino acids to about 75 amino acids, about 7 amino acids to about 70 amino acids, about 7 amino acids to about 65 amino acids, about 7 amino acids to about 60 amino acids, about 7 amino acids to about 55 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 100 amino acids, about 8 amino acid to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acid to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 20 amino acids to about 100 amino acids, about 20 amino acid to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 100 amino acids, about 30 amino acid to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 40 amino acids to about 100 amino acids, about 40 amino acid to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 50 amino acids to about 100 amino acids, about 50 amino acid to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 60 amino acids to about 100 amino acids, about 60 amino acid to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 70 amino acids to about 100 amino acids, about 70 amino acid to about 95 amino acids, about 70 amino acids to about 90 amino acids, about 70 amino acids to about 85 amino acids, about 70 amino acids to about 80 amino acids, about 70 amino acids to about 75 amino acids, about 80 amino acids to about 100 amino acids, about 80 amino acid to about 95 amino acids, about 80 amino acids to about 90 amino acids, about 80 amino acids to about 85 amino acids, about 90 amino acids to about 100 amino acids, about 90 amino acids to about 95 amino acids, or about 95 amino acids to about 100 amino acids, removed from its N-terminus and/or 1 amino acid to 100 amino acids (or any of the subranges of this range described herein) removed from its C-terminus.

In some embodiments, an active target protein (e.g., a supporting cell target protein or a hair cell target protein) can, e.g., include the sequence of a wildtype, full-length target protein (e.g., a wildtype, full-length supporting cell target protein or a wildtype, full-length hair cell target protein) where 1 amino acid to 50 amino acids, 1 amino acid to 45 amino acids, 1 amino acid to 40 amino acids, 1 amino acid to 35 amino acids, 1 amino acid to 30 amino acids, 1 amino acid to 25 amino acids, 1 amino acid to 20 amino acids, 1 amino acid to 15 amino acids, 1 amino acid to 10 amino acids, 1 amino acid to 9 amino acids, 1 amino acid to 8 amino acids, 1 amino acid to 7 amino acids, 1 amino acid to 6 amino acids, 1 amino acid to 5 amino acids, 1 amino acid to 4 amino acids, 1 amino acid to 3 amino acids, about 2 amino acids to 50 amino acids, about 2 amino acids to 45 amino acids, about 2 amino acids to 40 amino acids, about 2 amino acids to 35 amino acids, about 2 amino acids to 30 amino acids, about 2 amino acids to 25 amino acids, about 2 amino acids to 20 amino acids, about 2 amino acids to 15 amino acids, about 2 amino acids to 10 amino acids, about 2 amino acids to 9 amino acids, about 2 amino acids to 8 amino acids, about 2 amino acids to 7 amino acids, about 2 amino acids to 6 amino acids, about 2 amino acids to 5 amino acids, about 2 amino acids to 4 amino acids, about 3 amino acids to 50 amino acids, about 3 amino acids to 45 amino acids, about 3 amino acids to 40 amino acids, about 3 amino acids to 35 amino acids, about 3 amino acids to 30 amino acids, about 3 amino acids to 25 amino acids, about 3 amino acids to 20 amino acids, about 3 amino acids to 15 amino acids, about 3 amino acids to 10 amino acids, about 3 amino acids to 9 amino acids, about 3 amino acids to 8 amino acids, about 3 amino acids to 7 amino acids, about 3 amino acids to 6 amino acids, about 3 amino acids to 5 amino acids, about 4 amino acids to 50 amino acids, about 4 amino acids to 45 amino acids, about 4 amino acids to 40 amino acids, about 4 amino acids to 35 amino acids, about 4 amino acids to 30 amino acids, about 4 amino acids to 25 amino acids, about 4 amino acids to 20 amino acids, about 4 amino acids to 15 amino acids, about 4 amino acids to 10 amino acids, about 4 amino acids to 9 amino acids, about 4 amino acids to 8 amino acids, about 4 amino acids to 7 amino acids, about 4 amino acids to 6 amino acids, about 5 amino acids to 50 amino acids, about 5 amino acids to 45 amino acids, about 5 amino acids to 40 amino acids, about 5 amino acids to 35 amino acids, about 5 amino acids to 30 amino acids, about 5 amino acids to 25 amino acids, about 5 amino acids to 20 amino acids, about 5 amino acids to 15 amino acids, about 5 amino acids to 10 amino acids, about 5 amino acids to 9 amino acids, about 5 amino acids to 8 amino acids, about 5 amino acids to 7 amino acids, about 6 amino acids to 50 amino acids, about 6 amino acids to 45 amino acids, about 6 amino acids to 40 amino acids, about 6 amino acids to 35 amino acids, about 6 amino acids to 30 amino acids, about 6 amino acids to 25 amino acids, about 6 amino acids to 20 amino acids, about 6 amino acids to 15 amino acids, about 6 amino acids to 10 amino acids, about 6 amino acids to 9 amino acids, about 6 amino acids to 8 amino acids, about 7 amino acids to 50 amino acids, about 7 amino acids to 45 amino acids, about 7 amino acids to 40 amino acids, about 7 amino acids to 35 amino acids, about 7 amino acids to 30 amino acids, about 7 amino acids to 25 amino acids, about 7 amino acids to 20 amino acids, about 7 amino acids to 15 amino acids, about 7 amino acids to 10 amino acids, about 7 amino acids to 9 amino acids, about 8 amino acids to 50 amino acids, about 8 amino acids to 45 amino acids, about 8 amino acids to 40 amino acids, about 8 amino acids to 35 amino acids, about 8 amino acids to 30 amino acids, about 8 amino acids to 25 amino acids, about 8 amino acids to 20 amino acids, about 8 amino acids to 15 amino acids, about 8 amino acids to 10 amino acids, about 10 amino acids to 50 amino acids, about 10 amino acids to 45 amino acids, about 10 amino acids to 40 amino acids, about 10 amino acids to 35 amino acids, about 10 amino acids to 30 amino acids, about 10 amino acids to 25 amino acids, about 10 amino acids to 20 amino acids, about 10 amino acids to 15 amino acids, about 15 amino acids to 50 amino acids, about 15 amino acids to 45 amino acids, about 15 amino acids to 40 amino acids, about 15 amino acids to 35 amino acids, about 15 amino acids to 30 amino acids, about 15 amino acids to 25 amino acids, about 15 amino acids to 20 amino acids, about 20 amino acids to 50 amino acids, about 20 amino acids to 45 amino acids, about 20 amino acids to 40 amino acids, about 20 amino acids to 35 amino acids, about 20 amino acids to 30 amino acids, about 20 amino acids to 25 amino acids, about 25 amino acids to 50 amino acids, about 25 amino acids to 45 amino acids, about 25 amino acids to 40 amino acids, about 25 amino acids to 35 amino acids, about 25 amino acids to 30 amino acids, about 30 amino acids to 50 amino acids, about 30 amino acids to 45 amino acids, about 30 amino acids to 40 amino acids, about 30 amino acids to 35 amino acids, about 35 amino acids to 50 amino acids, about 35 amino acids to 45 amino acids, about 35 amino acids to 40 amino acids, about 40 amino acids to 50 amino acids, about 40 amino acids to 45 amino acids, or about 45 amino acids to about 50 amino acids, are inserted. In some examples, the 1 amino acid to 50 amino acids (or any subrange thereof) can be inserted as a contiguous sequence into the sequence of a wildtype, full-length protein. In some examples, the 1 amino acid to 50 amino acids (or any subrange thereof) are not inserted as a contiguous sequence into the sequence of a wildtype, full-length protein. As can be appreciated in the art, the 1 amino acid to 50 amino acids can be inserted into a portion of the sequence of a wildtype, full-length protein that is not well-conserved between species.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF DRAWINGS

FIG. 1A is a representative image of Myo7a/Iba-1 immunofluorescent staining of cochlear tissue of a cynomolgus macaque (non-human primate) following administration of a single Anc80-GFP AAV vector directly into the inner ear through the round window.

FIG. 1B is a representative image of Anc80-GFP immunofluorescent staining of the same cochlear tissue of the cynomolgus macaque as in FIG. 1A.

FIG. 1C is a representative image of a merged immunofluorescent staining of Myo7a/Iba-1 and Anc80-GFP of the same cochlear tissue of the cynomolgus macaque as in FIG. 1A.

### DETAILED DESCRIPTION

Provided herein are compositions that include at least two different nucleic acid vectors, where: each of the at least two different adeno-associated virus (AAV) vectors includes a coding sequence that encodes a different portion of a supporting cell target protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length supporting cell target protein; at least one of the coding sequences includes a nucleotide sequence spanning two neighboring exons of the supporting cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length supporting cell target protein.

Provided herein are compositions including two different nucleic acid vectors, where: a first nucleic acid vector of the two different nucleic acid vectors includes a promoter, a first coding sequence that encodes an N-terminal portion of a supporting cell target protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a supporting cell target protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length supporting cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length supporting cell target protein.

Provided herein are compositions including: a first nucleic acid vector including a promoter, a first coding sequence that encodes an N-terminal portion of a supporting cell target protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a supporting cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length supporting cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length supporting cell target protein.

Provided herein are compositions including: a first nucleic acid vector including a promoter, a first coding sequence that encodes an N-terminal portion of a supporting cell target protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a supporting cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length supporting cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length supporting cell target protein.

Provided herein are compositions including a single adeno-associated virus (AAV) vector, where the single AAV vector that includes a nucleic acid sequence that encodes a supporting cell target protein; and when introduced into a primate cell, a nucleic acid encoding a full-length supporting cell target protein is generated at the locus of the supporting cell target gene, and the primate expresses the supporting cell target protein.

Provided herein are compositions that include at least two different nucleic acid vectors, where: each of the at least two different adeno-associated virus (AAV) vectors includes a coding sequence that encodes a different portion of a hair cell target protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length hair cell target protein; at least one of the coding sequences includes a nucleotide sequence spanning two neighboring exons of the hair cell target genomic DNA, and lacks an intronic sequence between the two neighboring exons; and when introduced into a primate cell the at least two different vectors undergo concatamerization or homologous recombination with each other, thereby forming a recombined nucleic acid that encodes a full-length hair cell target protein.

Provided herein are compositions including two different nucleic acid vectors, where: a first nucleic acid vector of the two different nucleic acid vectors includes a promoter, a first coding sequence that encodes an N-terminal portion of a hair cell target protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a hair cell target protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length hair cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length hair cell target protein.

Provided herein are compositions including: a first nucleic acid vector including a promoter, a first coding sequence that encodes an N-terminal portion of a hair cell target protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a hair cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length hair cell target protein.

Provided herein are compositions including: a first nucleic acid vector including a promoter, a first coding sequence that encodes an N-terminal portion of a hair cell target protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a hair cell target protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length hair cell target protein.

Also provided herein are compositions including a single adeno-associated virus (AAV) vector, where the single AAV vector that includes a nucleic acid sequence that encodes a hair cell target protein; and when introduced into a primate cell, a nucleic acid encoding a full-length hair cell target protein is generated at the locus of the hair cell target gene, and the primate expresses the hair cell target protein.

Also provided are kits that include any of the compositions described herein. Also provided herein are methods that include introducing into a cochlea of a mammal a therapeutically effective amount of any of the compositions described herein.

Also provided are methods of correcting a supporting cell target gene defect in a supporting cell of an inner ear in a primate that include: administering to the inner ear of the primate a therapeutically effective amount of any of the compositions described herein, where the administering repairs the supporting cell target gene defect in the supporting cell of the inner ear of the primate.

Also provided are methods of correcting a hair cell target gene defect in a hair cell of an inner ear in a primate that include: administering to the inner ear of the primate a therapeutically effective amount of any of the compositions described herein, where the administering repairs the hair cell target gene defect in the hair cell of the inner ear of the primate.

Also provided herein are methods of increasing the expression level of a supporting cell target protein in a supporting cell of an inner ear of a primate that include: administering to the inner ear of the primate a therapeutically effective amount of any of the compositions described herein, where the administering results in an increase in the expression level of the supporting cell target protein in the supporting cell of the inner ear of the primate.

Also provided herein are methods of increasing the expression level of a hair cell target protein in a hair cell of an inner ear of a primate that include: administering to the inner ear of the primate a therapeutically effective amount of any of the compositions described herein, where the administering results in an increase in the expression level of the hair cell target protein in the hair cell of the inner ear of the primate.

Also provided herein are methods of treating hearing loss, e.g., non-syndromic sensorineural hearing loss or syndromic sensorineural hearing loss, in a primate identified as having a defective supporting cell target gene that include: administering to the inner ear of the primate a therapeutically effective amount of any of the compositions described herein.

Also provided herein are methods of treating hearing loss, e.g., non-syndromic sensorineural hearing loss or syndromic sensorineural hearing loss, in a primate identified as having a defective hair cell target gene that include: administering to the inner ear of the primate a therapeutically effective amount of any of the compositions described herein.

Also provided herein are methods of restoring hearing in a primate identified or diagnosed as having hearing loss that include: administering to the inner ear of the primate a therapeutically effective amount of any of the compositions described herein.

Also provided herein are methods of restoring synapses and/or preserving spiral ganglion nerves in a primate identified or diagnosed as having an inner ear disorder that include: administering to the inner ear of the primate a therapeutically effective amount of any of the compositions described herein.

Also provided herein are methods that include administering to an inner ear of a primate a therapeutically effective amount of any of the compositions described herein.

Additional non-limiting aspects of the compositions, kits, and methods are described herein and can be used in any combination without limitation.

### Gap Junction Protein Beta 2 (GJB2)

The GJB2 gene encodes gap junction protein beta 2, a member of the gap junction protein family. Gap junction proteins are specialized proteins, also called connexins, involved in intracellular communication. Mutations in GJB2 have been associated with hearing loss and deafness (Amorini et al., Ann. Hum. Genet. 79(5):341-349, 2015; Qing et al., Genet. Test Mol. Biomarkers 19(1):52-58, 2015).

The human GJB2 gene is located on chromosome 13q12. It contains 2 exons encompassing ˜5 kilobases (kb) (NCBI Accession No. NG008358.1). The full-length wildtype GJB2 protein expressed from the human GJB2 gene is 226 amino acids in length.

Various mutations in the GJB2 gene have been associated with hearing loss (e.g., non-syndromic sensorineural hearing loss or syndromic sensorineural hearing loss). For example, the c.35delG allele was found on 65.5% of patients from Eastern Sicily (Amorini et al., Ann. Hum. Genet. 79(5):341-349, 2015).

Additional exemplary mutations in a GJB2 gene detected in subjects having non-syndromic sensorineural hearing loss or syndromic sensorineural hearing loss, and methods of sequencing a nucleic acid encoding GJB2 are described in, e.g., Snoeckx et al., Am. J. Hum. Genet 77: 945-957, 2005; Welch et al., Am. J. Med. Genet A 143: 1567-1573, 2007; and Zelante et al., Hum. Mol. Genet. 6:1605-1609, 1997. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype GJB2 protein is or includes the sequence of SEQ ID NO: 1. Non-limiting examples of nucleic acid encoding a wildtype GJB2 protein are or include SEQ ID NO: 2. As can be appreciated in the art, at least some or all of the codons in SEQ ID NO: 2 can be codon-optimized to allow for optimal expression in a non-human mammal.

In some embodiments of any of the compositions described herein, the GJB2 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 1.

Human Full-length Wildtype GJB2 Protein
(SEQ ID NO: 1)
MDWGTLQTILGGVNKHSTSIGKIWLTVLFIFRIMILVVAAKEVWGDEQAD
FVCNTLQPGCKNVCYDHYFPISHIRLWALQLIFVSTPALLVAMHVAYRRH
EKKRKFIKGEIKSEFKDIEEIKTQKVRIEGSLWWTYTSSIFFRVIFEAAF
MYVFYVMYDGFSMQRLVKCNAWPCPNTVDCFVSRPTEKTVFTVFMIAVSG
ICILLNVTELCYLLIRYCSGKSKKPV
Human Wildtype GJB2 cDNA
(SEQ ID NO: 2)
atggattggggcacgctgcagacgatcctggggggtgtgaacaaacactc
caccagcattggaaagatctggctcaccgtcctcttcatttttcgcatta
tgatcctcgttgtggctgcaaaggaggtgtggggagatgagcaggccgac
tttgtctgcaacaccctgcagccaggctgcaagaacgtgtgctacgatca
ctacttccccatctcccacatccggctatgggccctgcagctgatcttcg
tgtccacgccagcgctcctagtggccatgcacgtggcctaccggagacat
gagaagaagaggaagttcatcaagggggagataaagagtgaatttaagga
catcgaggagatcaaaacccagaaggtccgcatcgaaggctccctgtggt
ggacctacacaagcagcatcttcttccgggtcatcttcgaagccgccttc
atgtacgtcttctatgtcatgtacgacggcttctccatgcagcggctggt
gaagtgcaacgcctggccttgtcccaacactgtggactgctttgtgtccc
ggcccacggagaagactgtcttcacagtgttcatgattgcagtgtctgga
atttgcatcctgctgaatgtcactgaattgtgttatttgctaattagata
ttgttctgggaagtcaaaaaagccagtttaa

A non-limiting example of a human wildtype GJB2 genomic DNA sequence is SEQ ID NO: 3. The exons in SEQ ID NO: 3 are: nucleotide positions 1-156 (exon 1), and nucleotide positions 3336-5469 (exon 2). The intron is located between each of these exons in SEQ ID NO: 3, i.e., at nucleotide positions 157-3335 (intron 1).

| Human Wildtype GJB2 Gene Sequence (NCBI Reference Sequence: NG_008358.1) | |
|---|---|
| (SEQ ID NO: 3) | |
| 1 | ggggtgcggt taaaaggcgc cacggcggga gacaggtgtt gcggccccgc agcgcccgcg |
| 61 | cgctcctctc cccgactcgg agcccctcgg cggcgcccgg cccaggaccc gcctaggagc |
| 121 | gcaggagccc cagcgcagag accccaacgc cgagaccccc gccccggccc cgccgcgctt |
| 181 | cctcccgacg caggtgagcc cgccggcccc ggactgcccg gccaggaacc tggcgcgggg |
| 241 | agggaccgcg agacccagag cggttgcccg gccgcgtggg tctcggggaa ccggggggct |
| 301 | ggaccaacac acgtccttgg gccggggggc gggggccgcc ttctggagcg ggcgtttctg |
| 361 | cggccgagct ccggagctgg aatggggcgg ccggggaagt ggacgcgatg gcaccgcccg |
| 421 | gggtgcgagt ggggccgggc gcgcgcggga ggggaaaaag gcgcgggcga gccgccagcg |
| 481 | cgaggtttgt ggtgtcgccg atgtcccttc ggggtactct agcgcagccg cctggctact |
| 541 | tgacccactg ccaccaaacg ttttaaattc accgaaagct tagcttcgaa gcaaagctcc |
| 601 | gtttcgccgg tgaagcagga agccttcgct gcaggaactg acctttacct cttggagcgg |
| 661 | cttctgcaga aaaatccccg ggcagagatt tgggcggagt ttgcctagaa ctaacgcgga |
| 721 | gccagccgat cccggcctac cccggggcca agatttcagt ggcttccctt tttcctaaac |
| 781 | acttcacgag ggtctgtttc cgggctgtgc tccccgccta gaaggaaaat ttttaggacc |
| 841 | cttgttcgcg aagaggtggt gtgcggctga gacccgcgtc ctcaggacgg ttccatcagt |
| 901 | gcctcgatcc tgccccactg gaggaggaag gcagcccgaa cagcgctcac ctaactaaca |
| 961 | gctgctgaga gctgggttcc gtggccatgc acctgggact gccttgagaa gcgtggtacg |
| 1021 | gccgtgtccc catgtgacct tagagtccct ttcgaaactg ctgtgcacag tcggtcacaa |
| 1081 | tttcagacac tggtgagaag ggtggaggaa ccctctgggg acagccaggc aaggtcgacc |
| 1141 | acccatcacc taagggtgga gaaatttaag gggtgaagag tcccttttgc cttttctgga |
| 1201 | tcctggtgat tcacctagtg tcttccctaa ggaactgaac caactcctcc gctggcctct |
| 1261 | ggcagccctc caggcggtgc aggatggcgt gggcccggta ggaagctgca tgtaaccgcc |
| 1321 | cagggtcggg aggccaggag ggcagctcct cctctgactt gaatattgaa aacaacttcg |
| 1381 | tcctgcttct gagcccctct taacccatga ccccctagcc cattggggag taaatcttaa |
| 1441 | tttactcctc ttcctgaaaa aggatcttta aaacaggtag cttcaactca agctttataa |
| 1501 | aataacaata tagggtttct cggaactgta tttttctcag ctgatggtaa ctggacaggt |
| 1561 | ctgtagaagg gtgtatgacc tgggtttggc aggtggaaga gggcaaagga taaacccctc |
| 1621 | ctcctgcagc cccatattct tggccaggtg tattgttgta aaccaggaga gagtttactt |
| 1681 | cggggagtat cctgttttcc actcagtgag ggccaatgaa gaatgtctaa ttccataaga |
| 1741 | tgcttttgtt aaaatcggaa tgttgctgtc ctcggtggtt ctgctgttgg gacgggactg |
| 1801 | gcctgagctg tgggtgctgt agcaggacaa ccagctcacc taagggcctc ccagtctgga |
| 1861 | ttatcaatgg gtcagtgctg aacctgggct aaaatattgt tttttccaat gatgttgtct |
| 1921 | ttcccaagct cagtgaagct aaatgtttca caggcctatg tcaatctgat gtaactttcg |
| 1981 | tggccacctc tctcctgtta gcctctgacc aaggtggcac tggatggttt ctgcctgacc |
| 2041 | ttggtgcccc gtggcagcga ctgtgggtca tgaaagacat tcactacgag cctgcttctg |
| 2101 | gagtccatca gaaaacggga tgcaacttgc ctaaaatgag gagaggagga tgcttttaag |
| 2161 | aaaaagaaga aggaggattc actaccagct ctgaagggtg gaaaagagat gattcatccg |
| 2221 | gattgtggag agggtggaat cttgtttagg agagcgttgg ttgtggcagg cagggtgtaa |
| 2281 | ctatgaatca gtgaagacaa ttcacatcct gggatgaaaa gaaggccatg ggctcacagg |
| 2341 | agattatcca ctggcctctc cacatccgct tgcagtaagg agtgtgggac tctcccaagc |
| 2401 | ttcagcgctg aactgcaatg cagtgacgtc gcttagctgg gccagtaacc gagggagttg |
| 2461 | aattttctgt cattttaaaa taatgtgtct tttaagaaac actttgaaat taaaaccaca |
| 2521 | gcccacaatt ataatgcact gttgcagcac ttatcaaaac agatatgcta actgagccat |
| 2581 | cagtgccagc ctgacagtga ggccaccaag ccatccacaa agcctacacg aaagtctgtg |
| 2641 | ctcacagtgg cttttctcca tgaagagggc attcctaacc tcttcctttc acgtaggagg |
| 2701 | aagcaaggtc ctttgtaaaa ttttaactcg gggtgcctca aatgtaaact taaccactgg |
| 2761 | taacaacagt ttcactgcta catgccacgt ctgtgaaaat tcattcaaga cattaaggaa |
| 2821 | agtggctcag cagagagact agacatctta tcctcacggt tctcctgtac ttggcctctc |
| 2881 | agcctttgag caaggttggc ccaagctagt atcggcccca gtggtacagc caaaacttga |
| 2941 | gactgcaaat ggatgcagct gttgaacgct gagtaacttc tgcagagtca ggaagaccca |
| 3001 | aggaagctct gcagaggatg caggggtacg gtcagaaccc ctgagtgcct ttcagctaac |
| 3061 | gaggacttta tgacactccc cagcacagca aatttttatg atgtgtttaa agattgggtg |
| 3121 | aattactcag gtgaacaagc tactttttat cagagaacac ctaaaaacac gttcaagagg |
| 3181 | gtttgggaac tatacattta atcctatgac aaactaagtt ggttctgtct tcacctgttt |
| 3241 | tggtgaggtt gtgtaagagt tggtgtttgc tcaggaagag atttaagcat gcttgcttac |
| 3301 | ccagactcag agaagtctcc ctgttctgtc ctagctagtg attcctgtgt tgtgtgcatt |
| 3361 | cgtcttttcc agagcaaacc gcccagagta gaagatggat tggggcacgc tgcagacgat |
| 3421 | cctggggggt gtgaacaaac actccaccag cattggaaag atctggctca ccgtcctctt |
| 3481 | catttttcgc attatgatcc tcgttgtggc tgcaaaggag gtgtggggag atgagcaggc |
| 3541 | cgactttgtc tgcaacaccc tgcagccagg ctgcaagaac gtgtgctacg atcactactt |
| 3601 | ccccatctcc cacatccggc tatgggccct gcagctgatc ttcgtgtcca cgccagcgct |
| 3661 | cctagtggcc atgcacgtgg cctaccggag acatgagaag aagaggaagt tcatcaaggg |
| 3721 | ggagataaag agtgaattta aggacatcga ggagatcaaa acccagaagg tccgcatcga |
| 3781 | aggctccctg tggtggacct acacaagcag catcttcttc cgggtcatct tcgaagccgc |
| 3841 | cttcatgtac gtcttctatg tcatgtacga cggcttctcc atgcagcggc tggtgaagtg |
| 3901 | caacgcctgg ccttgtccca acactgtgga ctgctttgtg tcccggccca cggagaagac |
| 3961 | tgtcttcaca gtgttcatga ttgcagtgtc tggaatttgc atcctgctga atgtcactga |
| 4021 | attgtgttat ttgctaatta gatattgttc tgggaagtca aaaaagccag tttaacgcat |
| 4081 | tgcccagttg ttagattaag aaatagacag catgagaggg atgaggcaac ccgtgctcag |
| 4141 | ctgtcaaggc tcagtcgcta gcatttccca acacaaagat tctgacctta aatgcaacca |
| 4201 | tttgaaaccc ctgtaggcct caggtgaaac tccagatgcc acaatggagc tctgctcccc |
| 4261 | taaagcctca aaacaaaggc ctaattctat gcctgtctta attttctttc acttaagtta |
| 4321 | gttccactga gaccccaggc tgttaggggt tattggtgta aggtactttc atattttaaa |
| 4381 | cagaggatat cggcatttgt ttctttctct gaggacaaga gaaaaaagcc aggttccaca |
| 4441 | gaggacacag agaaggtttg ggtgtcctcc tggggttctt tttgccaact ttccccacgt |
| 4501 | taaaggtgaa cattggttct ttcatttgct ttggaagttt taatctctaa cagtggacaa |
| 4561 | agttaccagt gccttaaact ctgttacact ttttggaagt gaaaactttg tagtatgata |
| 4621 | ggttattttg atgtaaagat gttctggata ccattatatg ttccccctgt ttcagaggct |
| 4681 | cagattgtaa tatgtaaatg gtatgtcatt cgctactatg atttaatttg aaatatggtc |
| 4741 | ttttggttat gaatactttg cagcacagct gagaggctgt ctgttgtatt cattgtggtc |
| 4801 | atagcaccta acaacattgt agcctcaatc gagtgagaca gactagaagt tcctagtgat |
| 4861 | ggcttatgat agcaaatggc ctcatgtcaa atatttagat gtaattttgt gtaagaaata |
| 4921 | cagactggat gtaccaccaa ctactacctg taatgacagg cctgtccaac acatctccct |
| 4981 | tttccatgac tgtggtagcc agcatcggaa agaacgctga tttaaagagg tcgcttggga |
| 5041 | attttattga cacagtacca tttaatgggg aggacaaaat ggggcagggg agggagaagt |
| 5101 | ttctgtcgtt aaaaacagat ttggaaagac tggactctaa agtctgttga ttaaagatga |
| 5161 | gctttgtcta cttcaaaagt ttgtttgctt accccttcag cctccaattt tttaagtgaa |
| 5221 | aatatagcta ataacatgtg aaaagaatag aagctaaggt ttagataaat attgagcaga |
| 5281 | tctataggaa gattgaacct gaatattgcc attatgcttg acatggtttc caaaaaatgg |
| 5341 | tactccacat atttcagtga gggtaagtat tttcctgttg tcaagaatag cattgtaaaa |
| 5401 | gcattttgta ataataaaga atagctttaa tgatatgctt gtaactaaaa taattttgta |
| 5461 | atgtatcaaa tacatttaaa acattaaaat ataatctcta taataattta aaa |

### Solute Carrier Family 26 Member 4 (SLC26A4)

The SLC26A4 gene encodes the Pendred protein, and has homology to sulfate transporters. Mutations in SLC26A4 have been associated with hearing loss and deafness (Albert et al., Eur. J. Hum. Genet. 14:773-779, 2006; and Qing et al., Genet. Test Mol. Biomarkers 19(1):52-58, 2015).

The human SLC26A4 gene is located on chromosome 7q22. It contains 21 exons encompassing ˜57 kilobases (kb) (NCBI Accession No. NG_008489.1). The full-length wildtype SLC26A4 protein expressed from the human SLC26A4 gene is 780 amino acids in length.

Various mutations in the SLC26A4 gene have been associated with hearing loss. For example, point mutations V250A, D266N, E303Q, F345S, D697, K715N, and E737D have been reported in Chinese and U.S. patients with non-syndromic hearing loss (Dai et al., Physiol Genomics 38(3): 281-290, 2015).

Additional exemplary mutations in a SLC26A4 gene detected in subjects having non-syndromic sensorineural hearing loss or syndromic sensorineural hearing loss, and methods of sequencing a nucleic acid encoding SLC26A4 are described in, e.g., Albert et al., Eur. J. Hum. Genet. 14: 773-779, 2006; and Qing et al., Genet. Test Mol. Biomarkers 19(1):52-58, 2015. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype SLC26A4 protein is or includes the sequence of SEQ ID NO: 4. Non-limiting examples of nucleic acid encoding a wildtype SLC26A4 protein are or include SEQ ID NO: 5 or 6. As can be appreciated in the art, at least some or all of the codons in SEQ ID NO: 5 or 6 can be codon-optimized to allow for optimal expression in a non-human mammal.

In some embodiments of any of the compositions described herein, the SLC26A4 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 4.

Human Full-length Wildtype SLC26A4 Protein
(SEQ ID NO: 4)
MAAPGGRSEPPQLPEYSCSYMVSRPVYSELAFQQQHERRLQERKTLRESLAKCCSCSRKRAFGVLKTLVPI
LEWLPKYRVKEWLLSDVISGVSTGLVATLQGMAYALLAAVPVGYGLYSAFFPILTYFIFGTSRHISVGPFP
VVSLMVGSVVLSMAPDEHFLVSSSNGTVLNTTMIDTAARDTARVLIASALTLLVGIIQLIFGGLQIGFIVR
YLADPLVGGFTTAAAFQVLVSQLKIVLNVSTKNYNGVLSIIYTLVEIFQNIGDTNLADFTAGLLTIVVCMA
VKELNDRFRHKIPVPIPIEVIVTIIATAISYGANLEKNYNAGIVKSIPRGFLPPELPPVSLFSEMLAASFS
IAVVAYAIAVSVGKVYATKYDYTIDGNQEFIAFGISNIFSGFFSCFVATTALSRTAVQESTGGKTQVAGII
SAAIVMIAILALGKLLEPLQKSVLAAVVIANLKGMFMQLCDIPRLWRQNKIDAVIWVFTCIVSIILGLDLG
LLAGLIFGLLTVVLRVQFPSWNGLGSIPSTDIYKSTKNYKNIEEPQGVKILRFSSPIFYGNVDGFKKCIKS
TVGFDAIRVYNKRLKALRKIQKLIKSGQLRATKNGIISDAVSTNNAFEPDEDIEDLEELDIPTKEIEIQVD
WNSELPVKVNVPKVPIHSLVLDCGAISFLDVVGVRSLRVIVKEFQRIDVNVYFASLQDYVIEKLEQCGFFD
DNIRKDTFFLTVHDAILYLQNQVKSQEGQGSILETITLIQDCKDTLELIETELTEEELDVQDEAMRTLAS
Exemplary Human Wildtype SLC26A4 cDNA
(SEQ ID NO: 5)
atggcagcgccaggcggcaggtcggagccgccgcagctccccgagtacagctgcagctacatggtgtcgcg
gccggtctacagcgagctcgctttccagcaacagcacgagcggcgcctgcaggagcgcaagacgctgcggg
agagcctggccaagtgctgcagttgttcaagaaagagagcctttggtgtgctaaagactcttgtgcccatc
ttggagtggctccccaaataccgagtcaaggaatggctgcttagtgacgtcatttcgggagttagtactgg
gctagtggccacgctgcaagggatggcatatgccctactagctgcagttcctgtcggatatggtctctact
ctgcttttttccctatcctgacatactttatctttggaacatcaagacatatctcagttggaccttttcca
gtggtgagtttaatggtgggatctgttgttctgagcatggcccccgacgaacactttctcgtatccagcag
caatggaactgtattaaatactactatgatagacactgcagctagagatacagctagagtcctgattgcca
gtgccctgactctgctggttggaattatacagttgatatttggtggcttgcagattggattcatagtgagg
tacttggcagatcctttggttggtggcttcacaacagctgctgccttccaagtgctggtctcacagctaaa
gattgtcctcaatgtttcaaccaaaaactacaatggagttctctctattatctatacgctggttgagattt
ttcaaaatattggtgataccaatcttgctgatttcactgctggattgctcaccattgtcgtctgtatggca
gttaaggaattaaatgatcggtttagacacaaaatcccagtccctattcctatagaagtaattgtgacgat
aattgctactgccatttcatatggagccaacctggaaaaaaattacaatgctggcattgttaaatccatcc
caagggggtttttgcctcctgaacttccacctgtgagcttgttctcggagatgctggctgcatcattttcc
atcgctgtggtggcttatgctattgcagtgtcagtaggaaaagtatatgccaccaagtatgattacaccat
cgatgggaaccaggaattcattgcctttgggatcagcaacatcttctcaggattcttctcttgttttgtg
gccaccactgctctttcccgcacggccgtccaggagagcactggaggaaagacacaggttgctggcatcat
ctctgctgcgattgtgatgatcgccattcttgccctggggaagcttctggaacccttgcagaagtcggtct
tggcagctgttgtaattgccaacctgaaagggatgtttatgcagctgtgtgacattcctcgtctgtggaga
cagaataagattgatgctgttatctgggtgtttacgtgtatagtgtccatcattctggggctggatctcgg
tttactagctggccttatatttggactgttgactgtggtcctgagagttcagtttccttcttggaatggcc
ttggaagcatccctagcacagatatctacaaaagtaccaagaattacaaaaacattgaagaacctcaagga
gtgaagattcttagattttccagtcctattttctatggcaatgtcgatggttttaaaaaatgtatcaagtc
cacagttggatttgatgccattagagtatataataagaggctgaaagcgctgaggaaaatacagaaactaa
taaaaagtggacaattaagagcaacaaagaatggcatcataagtgatgctgtttcaacaaataatgctttt
gagcctgatgaggatattgaagatctggaggaacttgatatcccaaccaaggaaatagagattcaagtgga
ttggaactctgagcttccagtcaaagtgaacgttcccaaagtgccaatccatagccttgtgcttgactgt
ggagctatatctttcctggacgttgttggagtgagatcactgcgggtgattgtcaaagaattccaaagaat
tgatgtgaatgtgtattttgcatcacttcaagattatgtgatagaaaagctggagcaatgcgggttctttg
acgacaacattagaaaggacacattctttttgacggtccatgatgctatactctatctacagaaccaagtg
aaatctcaagagggtcaaggttccattttagaaacgatcactctcattcaggattgtaaagatacccttga
attaatagaaacagagctgacggaagaagaacttgatgtccaggatgaggctatgcgtacacttgcatcct
ga
Exemplary Human Wildtype SLC26A4 cDNA
(SEQ ID NO: 6)
ctcagccttcccggttcgggaaaggggaagaatgcaggaggggtaggatttctttcctgataggatcggtt
gggaaagaccgcagcctgtgtgtgtctttcccttcgaccaaggtgtctgttgctccgtaaataaaacgtcc
cactgccttctgagagcgctataaaggcagcggaagggtagtccgcggggcattccgggcggggcgcgagc
agagacaggtcatggcagcgccaggcggcaggtcggagccgccgcagctccccgagtacagctgcagctac
atggtgtcgcggccggtctacagcgagctcgctttccagcaacagcacgagcggcgcctgcaggagcgcaa
gacgctgcgggagagcctggccaagtgctgcagttgttcaagaaagagagcctttggtgtgctaaagactc
ttgtgcccatcttggagtggctccccaaataccgagtcaaggaatggctgcttagtgacgtcatttcggga
gttagtactgggctagtggccacgctgcaagggatggcatatgccctactagctgcagttcctgtcggata
tggtctctactctgcttttttccctatcctgacatactttatctttggaacatcaagacatatctcagttg
gaccttttccagtggtgagtttaatggtgggatctgttgttctgagcatggcccccgacgaacactttctc
gtatccagcagcaatggaactgtattaaatactactatgatagacactgcagctagagatacagctagagt
cctgattgccagtgccctgactctgctggttggaattatacagttgatatttggtggcttgcagattggat
tcatagtgaggtacttggcagatcctttggttggtggcttcacaacagctgctgccttccaagtgctggtc
tcacagctaaagattgtcctcaatgtttcaaccaaaaactacaatggagttctctctattatctatacgct
ggttgagatttttcaaaatattggtgataccaatcttgctgatttcactgctggattgctcaccattgtcg
tctgtatggcagttaaggaattaaatgatcggtttagacacaaaatcccagtccctattcctatagaagta
attgtgacgataattgctactgccatttcatatggagccaacctggaaaaaaattacaatgctggcattgt
taaatccatcccaagggggtttttgcctcctgaacttccacctgtgagcttgttctcggagatgctggctg
catcattttccatcgctgtggtggcttatgctattgcagtgtcagtaggaaaagtatatgccaccaagtat
gattacaccatcgatgggaaccaggaattcattgcctttgggatcagcaacatcttctcaggattcttctc
ttgttttgtggccaccactgctctttcccgcacggccgtccaggagagcactggaggaaagacacaggttg
ctggcatcatctctgctgcgattgtgatgatcgccattcttgccctggggaagcttctggaacccttgcag
aagtcggtcttggcagctgttgtaattgccaacctgaaagggatgtttatgcagctgtgtgacattcctcg
tctgtggagacagaataagattgatgctgttatctgggtgtttacgtgtatagtgtccatcattctggggc
tggatctcggtttactagctggccttatatttggactgttgactgtggtcctgagagttcagtttccttct
tggaatggccttggaagcatccctagcacagatatctacaaaagtaccaagaattacaaaaacattgaaga
acctcaaggagtgaagattcttagattttccagtcctattttctatggcaatgtcgatggttttaaaaaat
gtatcaagtccacagttggatttgatgccattagagtatataataagaggctgaaagcgctgaggaaaata
cagaaactaataaaaagtggacaattaagagcaacaaagaatggcatcataagtgatgctgtttcaacaaa
taatgcttttgagcctgatgaggatattgaagatctggaggaacttgatatcccaaccaaggaaatagaga
ttcaagtggattggaactctgagcttccagtcaaagtgaacgttcccaaagtgccaatccatagccttgtg
cttgactgtggagctatatctttcctggacgttgttggagtgagatcactgcgggtgattgtcaaagaatt
ccaaagaattgatgtgaatgtgtattttgcatcacttcaagattatgtgatagaaaagctggagcaatgcg
ggttctttgacgacaacattagaaaggacacattctttttgacggtccatgatgctatactctatctacag
aaccaagtgaaatctcaagagggtcaaggttccattttagaaacgatcactctcattcaggattgtaaaga
tacccttgaattaatagaaacagagctgacggaagaagaacttgatgtccaggatgaggctatgcgtacac
ttgcatcctgaaagtgggttcgggaggtctctatgagcaaggaatacaagacaaaacttcctcaatgcatt
gactatttcttcagactcaaaacactcattcttttttctattaagccattgaaagagaagcactaagactg
cttctaggctttatttataaaataaacaccttatccctaacatgggcaaaatggctagaattattcagacg
atttggcagcgtccagggtaagctggtgttataatacgctgctgatctacatcacagatttgctaataatg
ttcacgtgggccctggcatatctctgttcagttagagtgagtgctgacccaacagcctctgtggtcaagcg
agtcacgaatgattaatcataaagaaaaatcagtttttgactgacctggatatccatgagctgcactgatc
accatgtaaggtcacatttagtaaatgctgaaataaaatgattaatgcatttatcaataaaagcctttgaa
aatactttggataataaattggagttttaaaaatgcaaatttgcttagtatctaataatgaagtgttatta
catatagccggaattgaggatctctttgatcctggaaatggtttacctaaaagctacagaaccaggccaat
atattttgaaatattgatgcagacaaatgaaataataaagagattttcatggtttataaaaatcttttttg
atatgataataatcatgatcacaactgagatcaaaaaaatatatgacagattattttgtttaaaaatgcag
ttttaattatcttagtctatagaaatgatcattgcatggaggcatgtataggtatgatctgtgtaaaatct
gacataaaaacagtgctattctgagtgaaaatttttttgatgtgcttacataaccatggtgattaaaatga
gtttatattttttctcaaaaattttagcagtgtgtaaagtaagtaatctttaactgaactctgaccactta
aaaaaaaatctaaaaattgaactacctatagtagtctgtgtttaaagtgaatttttaaagacaaagcattc
taaatgaactcaatataaaaacattcatttggaatgtacatactgaaaaatacaggtttttttgaccaaaa
gtttttatatcttttctttttatttatttttttcctaagtgccaacaattttctagatattatatacaaca
caggctttgatcttggggacttttcccatatatttcacactggagtgaatgaagttgtacttcatttctag
agaaaagttatacccaggtccccaattgagaatgtcttgcttgattgaaaacgacatcatcccttggtata
ctccagggattggtttcaggacccctgcatttaccaaaatttgtgcacactcaagtcctgcagtcacccct
gcctaaagatagaatggcttctctgtttttcttctgaaatacaaccagaaacaatgtgtctatttctgaaa
gaataggattaatgatcatacaaatgggttaatcctgaattctggttgtaaatctggttacagcataacta
ggattataatgctgcctcattttcacagcactacttgcttatattgacaacaaatcatctcgctaaagagt
gaatgtaggccaggcgcggtggctcatgcctgtaatcccagcactttgggaggccgaggcgggtggatcac
gaggtcaggagatcgagaccatcctggctaacatggtaaaaccccgtctctactaaaaatagaaaaaaaga
aattagcctagcgtggtggctggcgggcgcctgtagtcccagctatttgggaggctaaggcaggagaatgg
cgtgaacccgggaggcggagcttgcagtgagccgaggtcgtgccactgcactccagcctgggcgacagagc
aagactccgtctcaaaaaaaaaaaaaaaaaaaaaaaaagagtgaatgtaatagtcttgcagaaaatgaatg
aatacctttgttcaataaaggaaatatgcactgctcacttttttgaaggaaatgccaaagttacgttttac
aacaaggctagagtttgtaaattctgggttcatttgtgatgacataagtcagcaaactgcgggaatactgt
ctcttctatgtattttgtgaatagtaagcataattttagttttgtattatcaatgaaaatttcacttgaaa
ttaaagctgccttttgttatatttttaacctataggataagattccagtattgtatatgagttttaacaaa
ttaaaaaatcaaatcatgtacatttgaaaatatttgcacacatttaaaaataaatgtaaagttgtctttta
aactactcggatgtgtcctttctgaacaa

A non-limiting example of a human wildtype SLC26A4 genomic is SEQ ID NO: 7. The exons in SEQ ID NO: 7 are: nucleotide positions 1-221 (exon 1), nucleotide positions 1005-1171 (exon 2), nucleotide positions 2262-2801 (exon 3), nucleotide positions 11504-11614 (exon 4), nucleotide positions 13530-13714 (exon 5), nucleotide positions 14311-14475 (exon 6), nucleotide positions 22568-22720 (exon 7), nucleotide positions 22821-22903 (exon 8), nucleotide positions 28419-28566 (exon 9), nucleotide positions 29490-29603 (exon 10), nucleotide positions 33769-33846 (exon 11), nucleotide positions 33987-34082 (exon 12), nucleotide positions 35299-35405 (exon 13), nucleotide positions 37408-37477 (exon 14), nucleotide positions 39449-39541 (exon 15), nucleotide positions 40467-40562 (exon 16), nucleotide positions 41193-41423 (exon 17), nucleotide positions 43697-43751 (exon 18), nucleotide positions 49420-49565 (exon 19), nucleotide positions 51905-51988 (exon 20) and nucleotide positions 56348-57175 (exon 21). The introns are located between each of these exons in SEQ ID NO: 7, i.e., at nucleotide positions 222-1004 (intron 1), nucleotide positions 1172-2261 (intron 2), nucleotide positions 2802-11503 (intron 3), nucleotide positions 11615-13529 (intron 4), nucleotide positions 13715-14310 (intron 5), nucleotide positions 14476-22567 (intron 6), nucleotide positions 22721-22820 (intron 7), nucleotide positions 22904-28418 (intron 8), nucleotide positions 28567-29489 (intron 9), nucleotide positions 29604-33768 (intron 10), nucleotide positions 33847-33986 (intron 11), nucleotide positions 34083-35298 (intron 12), nucleotide positions 35406-37407 (intron 13), nucleotide positions 37478-39448 (intron 14), nucleotide positions 39542-40466 (intron 15), nucleotide positions 40563-41192 (intron 16), nucleotide positions 41424-43696 (intron 17), nucleotide positions 43752-49419 (intron 18), nucleotide positions 49566-51904 (intron 19), and nucleotide positions 51989-56347 (intron 20).

| Human Wildtype SLC26A4 Gene Sequence (NCBI Reference Sequence: NG_008489.1) | |
|---|---|
| (SEQ ID NO: 7) | |
| 1 | ctcagccttc ccggttcggg aaaggggaag aatgcaggag gggtaggatt tctttcctga |
| 61 | taggatcggt tgggaaagac cgcagcctgt gtgtgtcttt cccttcgacc aaggtgtctg |
| 121 | ttgctccgta aataaaacgt cccactgcct tctgagagcg ctataaaggc agcggaaggg |
| 181 | tagtccgcgg ggcattccgg gcggggcgcg agcagagaca ggtgagttcg ccctgaagat |
| 241 | gcccacaccg cccggcccgg gctccactcc cggggaggcc tcgagggttg cggatgggac |
| 301 | tcttaagtgg tcacggatca ggtgggcagg gggcagtaca gctttctttc tgagacgccg |
| 361 | agagcgaaca ggctgctcgg aaaacaggac gaggggagag acttgctcaa taagctgaaa |
| 421 | gttctgcccc cgagagggct gcgacagctg ctggaatgtg cctgcagcgt ccgcctcttg |
| 481 | gggacccgcg gagcgcgccc tgacggttcc acgcctggcc cgggggtctg cacctctcct |
| 541 | ccagtgcgca cctggagctg cgtcccgggt caggtgcggg gagggaggga atctcagtgt |
| 601 | ccccttccag ccttgcaagc gcctttggcc cctgccccag cccctcggtt tgggggagat |
| 661 | ttcagaacgc ggacagcgcc ctggctgcgg gccatagggg actgggtgga actcgggaag |
| 721 | cccccagagc aggggcttac tcgcttcaag tttggggaac cccgggcagc gggtgcaggc |
| 781 | cacgagaccc gaaggttctc aggtgccccc ctgcaggctg gccgtgcgcg ccgtggggcg |
| 841 | cttgtcgcga gcgccgaggg ctgcaggacg cggaccagac tcgcggtgca ggggggcctg |
| 901 | gctgcagcta acaggtgatc ccgttctttc tgttcctcgc tcttcccctc cgatcgtcct |
| 961 | cgcttaccgc gtgtcctccc tcctcgctgt cctctggctc gcaggtcatg gcagcgccag |
| 1021 | gcggcaggtc ggagccgccg cagctccccg agtacagctg cagctacatg gtgtcgcggc |
| 1081 | cggtctacag cgagctcgct ttccagcaac agcacgagcg gcgcctgcag gagcgcaaga |
| 1141 | cgctgcggga gagcctggcc aagtgctgca ggtagcggcc gcgcgggcct gcgtagagag |
| 1201 | aagcggagcg gggcgtccac gccttgggga gggaagggcg tccccagcgg gcgagagtgg |
| 1261 | ggtgcgggcg gcggagcccc tgggcgccag ctgcttctcc cagaggcccg actttcggtc |
| 1321 | tccggtcctc cacgccgccc ttctggtggg agggtggctc catcagtctc gggcccgaaa |
| 1381 | tgaacttacc tgggaaactc gcctttgggg agagtgggtt ctaggagccc cgtctctctt |
| 1441 | tttcctctct gaaggaaact tggagtgcct cttggggtac agtgggtccc tgttgccttc |
| 1501 | ttgggagctt gtttaaatga aatgaatagg gaaacccagc tcttgaccag gaggagtcct |
| 1561 | tgaaacactc aagctaagta ggcgggctac cattcagtta gagaccagga tgcaagctag |
| 1621 | aacccagggg agcgcggggt gtgccaagta cttcatcagc aggctgtggg acccctgggg |
| 1681 | aaagccaccc tcagtctcta aacccaaaca tgccgtaact agatgtcaca aacataaaga |
| 1741 | aattagagtt tctaaaacct ttcattatag aacatttcaa atatatgcat aagtagacgt |
| 1801 | agtagtataa tgaactcccc acccccaccg tttttaaccc atcactcggc ctcaactgtg |
| 1861 | atcaaatcca agttactctt gtttcttgta tctacaccta cttgctccct ccagtattat |
| 1921 | ttttaagcaa atctcatata gcatactgtc tttatgtatt ttactgcata tctttaaaga |
| 1981 | ctcttaaaaa atataaccac atttcatgat cacaccttaa tatctaaaaa taacctccta |
| 2041 | atatcaaaaa ttgagtcatt attaaaatgt ccaatcgtcc cataaatgcc ttaattaaaa |
| 2101 | aacaatttat tcaaatcagg agttatacca cctacttttg atatttttct ttcatctgta |
| 2161 | ggtcactgaa tttaaaaact accaagtagc aaggtataag gtatacattc ctcatgctaa |
| 2221 | gatttttgta aaaactagct ccaggcttgt attgccaaaa tatactcagt gtgtttatct |
| 2281 | tctttaaaga aaataataat aataaggcgc ctggattagg agtctgaaaa gtaatctcca |
| 2341 | tttagagact ccacatccat cagtctctgg ctggaccaga aaataggttt ttgtgtagga |
| 2401 | atattttttc agaggattaa aaatgtgagc gaggggtggg acacttaatc ctgtgttctc |
| 2461 | tagaagagtg catttaaaag gatagataca gttcttggca aaagcatggt aagcacttca |
| 2521 | gggttattat tttccaggaa atacttatcc tttttccaaa tagttataaa catcagcaga |
| 2581 | atccagttca taactttgtg atttgcaaat tggttgtgac tgagattgga ttgaaaaccc |
| 2641 | agttttcttg ctttttgaca gttgttcaag aaagagagcc tttggtgtgc taaagactct |
| 2701 | tgtgcccatc ttggagtggc tccccaaata ccgagtcaag gaatggctgc ttagtgacgt |
| 2761 | catttcggga gttagtactg ggctagtggc cacgctgcaa ggtaagatgt tggcagattg |
| 2821 | agagttctgg tctccagcag gagtttaaca cttctcccca gctaccatag gtctgtgaca |
| 2881 | gatggttgct tacccttcaa ggcctgtatc tttcctgtag agccccttag tggagagagt |
| 2941 | cacctctctt ctccccttcc ttagagttct cttcctggga aactgctgcc ccactaggtg |
| 3001 | cagaggtcca atttagaggc atatactagg cagtggcttc tcaatttttt taaattttat |
| 3061 | tttatttgag acgggggctc gctctgtcac ctaggctgga gtgcagtggc gcaatcctgg |
| 3121 | ctcactgcaa gctccgtctc ccgggttcac gccattctcc cgcctcagcc tccccagtag |
| 3181 | ctgggactac aggcgcccgc cactacgccc ggctaatttt ttgtattttt agtagagacg |
| 3241 | gggtttcacc gggttagcca ggatggtatc gacctcctga cctcgtgatc cgcctgcctt |
| 3301 | ggcctcccaa aatgctggga ttacaggcgt gagccactgc acccagccag cttctctatt |
| 3361 | ttcattgacc acaattcaat aagaaatgtg taaagagttt caagtcaaga tttaaaaaaa |
| 3421 | aaaaagaaat gtgttacatc ctgatatata catatatatc taaagttcct gtgaaatatt |
| 3481 | tattataaca atgtgctaat ctttcacttt gttctattat gcttaggttt tttaaatgat |
| 3541 | gattgcagca cattaaatta ttttatatcc acacgtggat cttttttgtt ggtttgtttt |
| 3601 | gctttgcttt ttagagatac agtcttgctc tgtcacccag gctggtgtgc agtggcacga |
| 3661 | tcttggctca atgcaacctc tctgcctctc gggttcaagt gattcttgtg cctcagcctt |
| 3721 | ctgagtagct ggggctacag gcatgtgcca ccatgtccag ctaattactg tatttttagt |
| 3781 | agagatgggg tttcgccatg ttggccaggg tggtctggaa ctcctaacct caagtgatcc |
| 3841 | gccctccttg gtatcccaaa gtactgtgat tacaggcatg agccaccatg cccagcctcc |
| 3901 | cacacgtgga tcttgaggtg tagtttgaaa ctggcttaag gaaaagtgaa gggagcttcc |
| 3961 | tagcctggac tccacatgaa tgttttgcct gcccctcatc cctttttaaa aaaattcatc |
| 4021 | ctcttatttt aatgtgtggg aaaatgtgcc aacataggtg gttatttgtt cttttataaa |
| 4081 | ctgagttcat accataatat tcccataagc atcttagaaa tctgttgtat agttggtgcc |
| 4141 | tagcagcatg gcattctcct ctgtccccta tccccaccat ctcgtttctg ccccaccccc |
| 4201 | aacaccctga ccaatttccc aaagaccctg aatcatgaaa acagaaatat cgttatttat |
| 4261 | tataatttgt gaaacaccta atatgttcca ggcactaggt atgctgacac gaataagatg |
| 4321 | catcctctgc ttccaaggaa ctcatagtcc aatggagaaa gagaaaacaa ttcaatgtga |
| 4381 | taagggctgt ggcaggggag tgcaaattac ttgctaaggg agtgcaaatg aggttcactc |
| 4441 | aaagcggtct taggggatct tgaatagctt cctggaagtt gaatgtcaaa gctgagtttc |
| 4501 | cctgatggct aagtctgagt ttgccaagtg atgggaagta gggtggggtg gggatggctt |
| 4561 | gaaaatagac tatttcagaa gctagtaatg aatgttgagc aatattgtga gctaaaattt |
| 4621 | ggctatggag ctttcaagat tgctcagatc aggccaggta gatgcccgaa gagaaggagg |
| 4681 | atggcctatg aacccagggt ggagcctcag cctgatcaag catgttctta atcacaaagg |
| 4741 | tggacgggag agagactagg atgggcbgtg tggttcctaa gttaaacaat gccaagtatt |
| 4801 | ctacactgtg ctctaagggc tgtgacatgg tgacaccctg tgactcctgg gggacaggaa |
| 4861 | ccatgcctca ttcatgtttg ttcagcaggt gcctagcaca gagacaggca cataatagat |
| 4921 | ggtatattta gtgaaaaaga ttgaattgca ttcctgataa tgaatcttct ttatatataa |
| 4981 | aaaatggtgg ttcatgctcc atgtctcccc agttttcata gacaagtatc ttataagaat |
| 5041 | tcattttgtt tataagcatg agacactatg ctaagtaaga atgttgccag ctatctcatt |
| 5101 | taatcctcat aacaatcctg ctaggtaggt ctattattat ttcattttca gtaggtgaaa |
| 5161 | aaatctatta aatcttgccc agggtcacgt ggctggtaag tggtacagct gggattttaa |
| 5221 | tccggtgtct attgtgtagt tccttacttt atgggtaaga tgcttttcta gaaacattat |
| 5281 | ctctataaag ttttctgtgt aaggtgaatt tctgggaggt taatctggct cagttacaca |
| 5341 | tccatttatc attcattcac ttaataaata tttataaact aattctgtag accaggtatc |
| 5401 | cagccatgtg cactcagtgg ttcattaaca ggcacaggtg ctgacttcat ggagctttgt |
| 5461 | aaaattattt tgaggggtag ggatgggaat taaataagta gataaataga aaaataatta |
| 5521 | tagattgcaa tatctattgt ggtggattaa atatactgca atagggatta atttgaaagg |
| 5581 | tggatcttta tttttatatt tttttgagac agggtcttgc tctgttgcgc aggctggagt |
| 5641 | gcagtggtga gatcacagct caccacagcc ttggcctcct gggcccaggt gatcctccca |
| 5701 | tctcagcctc gccagtagct ggaactacag gcatgtgcca ccatgcttgg ctaatttttg |
| 5761 | tattttttgt agagacaagg tttcaccatg ttgcccaggc tggtctggaa ctcctaggct |
| 5821 | caagttatct gcctgcctcg gcctcccaaa gtgctgggat tagaggtgtg agccattgtg |
| 5881 | cccagccagg aagggaggtc tattttagat ataatggtca aggaagaact tactgagaag |
| 5941 | gtgaaattta gtaaaagaca agagaatgag aaggagacag ccatgtgata agcagaaaaa |
| 6001 | aagagtgtac caggtaaagg tgcaaaggcc ctgaggccag aaaacttagc attttccagg |
| 6061 | gaggaagagg aagaattgta gtgtggatga gtgcagtggg ggagggtgag taaaccatga |
| 6121 | aataaactga ggcaggactg ggattaggat gaggtaaggg aggcactcac tctcaggatg |
| 6181 | cctctttaaa tattgtaccc taggaaactc atttgcctca ccctattcct ggccctgtgg |
| 6241 | ggatattgct gtgaattaaa gggagaatgg gtgtgtcagc ttgaaccaga tgatgcaggc |
| 6301 | tatgaagaga gctgcaagat aggaatatat tgtgcagcaa gaatcactga cacataatga |
| 6361 | tgagtgtgga tgaaaatcac tgatgcctcc aaggttgttg gcttgagcaa ccggatagat |
| 6421 | cattacatca tttatctaaa tggcaaagac aggctgagga gcaggtttgg gataggaaat |
| 6481 | caagtttcct tttggatatg ctaagtttga gacacagggt gagacatcta agtggagatg |
| 6541 | ccacctagag aagtgggagc tactgcaagt gtgaaggtta gaggagaggt ctaggctaga |
| 6601 | cataaaaatc aggaaagcat tgctgaagag gtgatattta caagagtggg aatgcctgtg |
| 6661 | attacctagg aagaaagggc aggtgtagaa gaaaagagag ccagagtact tagaagttag |
| 6721 | gaaggcaagg agaggggcag ccaagaacct gagaaagcct ggcgcaggaa gttgaagaca |
| 6781 | aagaacaacc aaaggaatgt gggatcagaa gccaagagag aaggtttaaa aaaaaaaaaa |
| 6841 | aaaaaaaaaa agcatagagg ggctgagagt gttgaatttg cctgaggggt ggggtatgat |
| 6901 | gaaggattac agactgcttt taagaagttt tgccatgaat cagagggaga taagagatga |
| 6961 | agacagagtg aagggagaat ttgtttttct taatatggga gatactagaa cgtatttgtg |
| 7021 | aaccgatagg aatgacctag tagaaagaaa gagatctgtg gtgcaagaag tacaagagaa |
| 7081 | ataactagaa gcaaagtagt aaaaaggggc aaacgtggag gggtttgaga atggaactag |
| 7141 | gagaactact ccttaaaggc aggagggata tttcttccat tttcaaagga agatagccca |
| 7201 | aatggtgcag atgcagacgg gtttgaaatt ttgatgaaaa gtaacttggg agcatctcat |
| 7261 | gcattctgtg ttttcgatta aatgcagaat atgtccctta actttgagtc tggtggagaa |
| 7321 | gggtttgggc agattgaggt aagtagggga ggtttgaata gttgtctcaa ggagaggaaa |
| 7381 | ggcacacccc ctgaggaaat gtaactgggt ccccaggcag tgttgagtgc ccatctagca |
| 7441 | cttgtggtca cacattttac atgaaattgg ttagcccaga tgtagatgcc ttcagtccag |
| 7501 | tttatctagc tgctcaaagg ctctcatgga taaggtagat gtttgaggtc agccaaattg |
| 7561 | gggttttgcc agacaagaaa aggaaggaaa gagagagtga aaaaagggag ttaagaatat |
| 7621 | ttgcaaaatt ccatggaatc tgtgtgggca agggtggaag tgaagacagg aaggtgtgga |
| 7681 | ggatagagag aaagtggatc ggttaattaa ttagaagcct cgtgagcaca aataaatgtt |
| 7741 | gcaataagga ttctgcagta gatgagctgg aaggtttgga ggctgtagct ggagagaggc |
| 7801 | tcaaaattca gattttggag gtggtggtgt ggtttccagt gatgacaatg tccatgccac |
| 7861 | aaccactggc tgagcagctg agtagtagaa caactccgag aagaccagct caggttactg |
| 7921 | ggaagccagg aagttgctaa ggatgatggg atctttgtga tttttgttat taaaacaaat |
| 7981 | tcccatcaga cagcaaaaga tcgatgcaac ccttgtagtg ctatttttat tcacaacttt |
| 8041 | aaggagtctg ctgtctgctt tctccttttg acttttatgt atttgtttgt ttaaacaaca |
| 8101 | gaaatttatt tctgcaacat gaaggtgcct gtagattcag tttctggtga cagcccgtca |
| 8161 | ccttgctgcg tcctcacatg gtggaaggct ttacctttag aagaaattta aatttaatat |
| 8221 | aaatttcaac tgtttattag cacctgcttt gccagtattt gtcaccatgc tataaatcat |
| 8281 | gactaccaac agagaaattc tatcatttaa atcaatggct cagatttctt ctaatttttt |
| 8341 | ttcttttttg agactctttc tcacacaggc tggagtgtag tggtatgacc ttggctcact |
| 8401 | gcaaccactg cctcctgggt tcaaactatc cttgtgcctc agcctcctca gtagctgaaa |
| 8461 | ttacaggcat gtaccaacac ccccagctaa tttgtgtatt tttagtagag acgagatttc |
| 8521 | accatgttgg ccgggctggt cttgaactcc tggcctcagt gatcttccca cctcagcctc |
| 8581 | ccgaaatgct aggattacaa tcatgagcca tcacacctag ccccagattt ctttacaaat |
| 8641 | tgtgtaattt taaaactgtt gtaccaagtt tctttataat aaagatgatt ccagtatatt |
| 8701 | tgagtagtag tggtccctaa atgggacact ctgtatgaaa actgaaaagt tccccttttc |
| 8761 | caggcatatt aagaactgca gagcccagtt tggggaaatg gaagtttgag aaggacttga |
| 8821 | aaaatgtcca tatgaaatag acaagttgtg ctttcatccc ctgctccttc ttttcttatc |
| 8881 | ttccttctgt ggagtcacct tcctatttta ttccatgctg ttgggactac tgactattgc |
| 8941 | aattcctgtt tgtaatttac atattaaaac aattgtacta atagatatat ttttctagaa |
| 9001 | tacaaactcc actagggcag agactttgtt ttaacagtgc ctgacacatg gtaggtgcat |
| 9061 | aatcaactgt tgttgaatga atgaatgctt attgtagaaa actagaaaat acatataagg |
| 9121 | aacatgaaaa aatacctcca ctacaaacta gaagtctcct ctgatcctac catacagata |
| 9181 | acccattgat atttcagtaa atatcttact aggcacataa atacttatct atataaaagt |
| 9241 | ataatgaaat gtctggaaat aataagcacc aaattcagga taattgttgc cttctgaggg |
| 9301 | tagagggagg aaagaggatg tgattgagga agagtatact gagagtttcc gctgcattgg |
| 9361 | cagtaattta tttcttaagc tagatgatgg atacatgaaa gtttattata ttattcattg |
| 9421 | tgtattgtgt ttcttgtata tgtaaattac ttcatatttt attcaaaata cctccagatt |
| 9481 | tttttttttt tttgagatgg agtcttgctc tgttgcccag gctggaatgc agtggcgcaa |
| 9541 | tctcggctca ctgaaacctc cgcctcctgg gttcaagcag ttctctgcct cagcctcctg |
| 9601 | agtagcbggg attacaggtg cctaccccca tgcctggcta atttttgtat ttttagtaga |
| 9661 | gacggggttt caccatcttg gccagggtgg tcttgagctc ctgacctcct gatccaccca |
| 9721 | cttcggcctc ccaaagtgct gggattacag gtgtgactca ccgcgcccgg tccctccggc |
| 9781 | tttttttaat gcgtacactc ccacaggctg gagtgggagt gtatttttta caaaagagga |
| 9841 | agcatacata cctatcagtt ttgaatcttg attttactca cttttttccc tcttacttca |
| 9901 | catctctcca cgttactgaa tgtacttctg agcatcacat caatttcatt tcatagaatt |
| 9961 | acgaagtatg aagcataact tagttaacca cttccctgct tggggacatt catctgataa |
| 10021 | gtaatattga gtgtgtgggg gccagcatat tcaggcbgtt gccattttta ataaactact |
| 10081 | ataaatgtaa ttccttatta atgactcaag agtgaaaagg cttgattttc tctcattgtg |
| 10141 | agtgagtgaa gggtgaggag gtaaaacgga gtaagatatg ctggctacct aagtatttaa |
| 10201 | ccaaggaaaa gaatgtcatt atcctcttct aaccctcatg taaactagaa tgttgatttc |
| 10261 | tctatagcca ggcattaatg ggtctggggg ctgctgccat tgcctagtgg aggagttggt |
| 10321 | caacttcatc agggtagagt tgtcttgtct gctgctgagt ataattatca tattttttct |
| 10381 | ttgggtctaa cttttcttcc ccaccaaaaa aggagaaagg gcagtgtaac aatgactgtg |
| 10441 | tccctcagaa cgcagagaaa tattgagctc agacacaact gcctctgtgc aaaactggtc |
| 10501 | tgagcctttg gagtgaattg accaaaactt ttttttttct tttttagaca taaggtcttg |
| 10561 | ctctgttgcc caggctggag tgcagtggca cgatcatagc ttgctgtaac ctcaaactcc |
| 10621 | tgggctcaaa caatcctcct gcctcagcct ccttaataac taggactaca ggcgtgaacc |
| 10681 | aatatgctgg gctcattcat tttattgtag agatggagtc ttgctatgtt ttccaggctg |
| 10741 | gtctcaaact cctgggctca actgatactc ctgcctatgg cttcccaaag cactgggatt |
| 10801 | ataggtgtag gacaccaggc ctgaccaagt ttttaatcaa catttgactt gattgttttt |
| 10861 | tcccactgat ctgattggtg gaagactgaa ttactgatca actgagtcca ttcatttgag |
| 10921 | caggtgatgt ttgtagatgc aatcattgtt ccaggtgatt tggatcctgt tctgcatcct |
| 10981 | agcatagcca gtctgtatgc aagataagga gaccaactca ggacaggctg atctgttgaa |
| 11041 | actgtaaatt gagagtatct gtgtaaaggc ccttctttct gcagtagaaa gacagatgct |
| 11101 | cctctacttc tcatgagtta catcctaata acccattgca agttgaacat attttaagtc |
| 11161 | aaaaatgcac ttaatacacc taacctatgg aacatcatcg cttaggctat cctacctgaa |
| 11221 | atgtgctcag aacacttaca ataacctaca gttgggcaaa ataatctaac gcaagcctat |
| 11281 | tttataataa agtattgaat atctcatgta gtttactgaa taatgtactg aaagtgaaaa |
| 11341 | acagaatggt tgtatggtta cttaaagtat ggtttctact gaatgcatat tgcttttgca |
| 11401 | tcatcataaa ggcaaagtca taagtggaac cattgtaagt tgaggacttt ctgcatactg |
| 11461 | taactttggt ttgtgaatgt aatcactttg catgtgcttt cagggatggc atatgcccta |
| 11521 | ctagctgcag ttcctgtcgg atatggtctc tactctgctt ttttccctat cctgacatac |
| 11581 | tttatctttg gaacatcaag acatatctca gttggtaatt ataagtatat tttacaatta |
| 11641 | tatttgctca tgtttaaagt gttttggcta tattaagtgc attatacctc tattaggttg |
| 11701 | gtgcaaaagt aattgcggtt ttcacaatta tacttttaat tgtgaaaacc gcaattactt |
| 11761 | ttgcaccaac ctaatatatc tgtgttaatg ttgtcaggga aatgggattt cagtgttttg |
| 11821 | cctgcttttt ctattcactg atgttaggta acttttttaa tgaagtggaa aaataaaaaa |
| 11881 | actgaaaatg acagcctact ttaacatttt agcatgtttt gctttttaaa aacgtgatca |
| 11941 | atttgactcc atttttggag tcataatgac agggaaaaaa tgacacaagg tctttgaacc |
| 12001 | taccatttta ctggattaac ttagaaatta ctaagtatgt tcatcaaata catgttcatg |
| 12061 | atttaattaa agagcatact tattttaaat cactaatatt agaagactgg caaaatctta |
| 12121 | ccaaaacaat ttagaagcta agcttggctt tctttcttgg tatggcttat aataagtttt |
| 12181 | cctgctattc ctaaaatttg aattctcgtt tgttagaatg tcctagtaaa tgagtgtttt |
| 12241 | aatgctattt tgtgactttg acatttatac tagagaaaat tttgctccta catccatctg |
| 12301 | ctggctagac atgtctacct cgatgatcct cttgaaataa atgcaaccat ctactttatc |
| 12361 | cccaacccat gtttttgtcc ttagttgtat ttccttagaa ggagatcaga aagaggtttt |
| 12421 | gattgaaaga acttccacac cttggagaat caatcagtgt tgagtgcttg actagatgag |
| 12481 | gaacatggaa gcbggttttt ctctttggta ttctatacat tgagatattc caggtgactg |
| 12541 | gggttgggtt ttgagacctc aaaaccataa agccttccat gatgagaggg tagaatcatc |
| 12601 | atcatcgtca tggataaaaa taacagtagt gaacggagca attgctgaaa aaaaaatatt |
| 12661 | ttttaaaata taaaatctta cagattgaca tttgatatga aaaaatgttt tgtcttacaa |
| 12721 | aaagagaaag aaacttcata ctccctttgc tgttttttta atcctaactt cgaccctgtg |
| 12781 | atattgatca aggtactcag atccacagag atcaggacat tggacactct aggaaattaa |
| 12841 | tgagattata tgaaataaaa gttgagcgga acaagtaaac actcaatgta tgtgctacct |
| 12901 | ttgccagaaa acctttcctg acaactctca tccctgtccc caccacccat acacatgagg |
| 12961 | atcacacaca gacaggttag acgacctttt gatatactca cgaagcatcc tgaatgttgt |
| 13021 | attctaataa ctacctacgt gtctctctcg cctatttcta ggtcatgaat gcctcgagat |
| 13081 | atctgcgttt ttagcttctt tttgttgttg ttgagacaga atctggctct gtcacctagg |
| 13141 | ctggagtgcg gtggcgtgat ctcagctcac tgcagcctcc acctccccag ctcaagcaat |
| 13201 | ccccccacct cagcctcccg agtagcbggg accataggca cgcaccacca cacccagcta |
| 13261 | atttttgtct tattttggta gagatggggt tttactatgt tgcttaggct ggtctggaac |
| 13321 | tcctgggctc aagcgatctt cccacctcag tctcccaaag tgctgcggtt acagatgtga |
| 13381 | gccactgggt ccggctcagc ttctttcgtg aacaaacaat attttcctag tcacagctaa |
| 13441 | atcttttata cattttttaa accctatgca gacacattga acatttgtga ttaataactg |
| 13501 | attaattgtt agagactttt tttccccagg accttttcca gtggtgagtt taatggtggg |
| 13561 | atctgttgtt ctgagcatgg cccccgacga acactttctc gtatccagca gcaatggaac |
| 13621 | tgtattaaat actactatga tagacactgc agctagagat acagctagag tcctgattgc |
| 13681 | cagtgccctg actctgctgg ttggaattat acaggtaatg aacttacaag taaaatatag |
| 13741 | atggatgtaa tttttatttg aaattaactt taaagcatat agacttaaag attctactaa |
| 13801 | aaacaaaaca aagtaatttc ctggaaccca aaattatttt ctaaattacg ttgttttagg |
| 13861 | tcaggtgcta aaatagtaag caagacccca cttattaagg ctcacttatc atcaaagtca |
| 13921 | gagacagaaa aaagcacaga agaacatgtg tgattcaatt gaggatgaga gaagggaaat |
| 13981 | atacgggaat ccataaagga gaaaaagtgt tctggtgagc ggagacacag gaccgaaagc |
| 14041 | cacataaata agctctgggt ttttgctttt ctaagtacat gtatagaaat acttcaaggt |
| 14101 | ttttattaca cttagttttc aaattttaga gtggtggagg aaggggagtg atagggtatt |
| 14161 | aagaaattca tatttttttc taccagtatt tttgtgctat aggcaggcta ctagtgtttt |
| 14221 | cattggtatt aagcttgatg taatatttcc agagagtagg tttctatctc aggcaaacat |
| 14281 | ttaatttttc tttccttttc cttatcgtag ttgatatttg gtggcttgca gattggattc |
| 14341 | atagtgaggt acttggcaga tcctttggtt ggtggcttca caacagctgc tgccttccaa |
| 14401 | gtgcbggtct cacagctaaa gattgtcctc aatgtttcaa ccaaaaacta caatggagtt |
| 14461 | ctctctatta tctatgtaag tgttgcttct tgctccaggg atgggtcact gttcattcca |
| 14521 | gaaacaattg tattcattct ctgagtctgg gccaggcgtg gtggctcaca cctgtaatcc |
| 14581 | cagcactttg gaaggccgag gtgggcagat tgcttgagcc caggagtttg agacgtgaga |
| 14641 | cctcatctct taaaaaaaaa aaaaaaaaaa aagaaagaaa gaaaagaaaa gaaaaagaaa |
| 14701 | gaaaaaatcc aaaaatccga aaatttgctg ggtgtggtgg tgcacacttg tagtctcagc |
| 14761 | tacttgggag gctgaggtgg gaagatctcc tgagccctgg aggttgaggc tgcagtgagc |
| 14821 | tgtgatcgcc ccactgcact ccaccctggg tgacagagca agaacctgtt tcaagcaaaa |
| 14881 | acaaaatcaa aacaaaacaa aacttgagtc tgggagcacc cacatttctt tctttctttc |
| 14941 | tttttttttt tttttttgag acagagtctt actctgtcac ccaggctgga gtgcagtggc |
| 15001 | atgatattgg ctcaccacaa cctccacctc ccgggttcaa gcgattctcc tgcctcagcc |
| 15061 | tcctgagtag ctgggattac aggcgcccgc caccacaccc agctaatttt tgtatattta |
| 15121 | gtagagacag ggtttcacca tttgggccag gcbggtcttg aactcctgac cttgtgatcc |
| 15181 | acccacatcg gcctcccaaa gtgctgggat tacaggcgtg agccaccgcg cccggcctgg |
| 15241 | gagcacccac atttctctat gcatactttg ggagccattg aaaacatttg gttgcttctg |
| 15301 | tcagaaatac tattgtactt aaaaacatta atgaaaacaa agtggtctaa tgaagtaaag |
| 15361 | gctgctctgg aatgtaacag acttgatttc tagcctcagc tttgtctcta acaggtaagt |
| 15421 | gaccctggat acatcatgta gggtgctaat tgaaagctca gttttagaac tggatggacc |
| 15481 | ttgattcata ttctggctct actgtctact agccatgtga gccaatcacc atgcctcagt |
| 15541 | ttccttctct gcaaaattgg gttagtaata gtatatgcct cattggattg ttaaataaga |
| 15601 | atagataagc acttggccca ggggcbggta cataacaagt actctaaata aaggagttat |
| 15661 | tttgaaatga ttatttcaag ccatcttttc ttttttattg gtgaaatgaa atgattggag |
| 15721 | atgtatctct aaaagctttt tctaaccata agagtccgta aaatgcataa tgtaaatgtc |
| 15781 | tcaacaatta taaatgaaaa ggaacattag attcagagat gattcaccat gcaaaagaaa |
| 15841 | tgcaaaagca ggatgtagat cacactaatt agatttgaaa aaggttttgg atctaaaaat |
| 15901 | gtgttctaca taaagcatgt tattgatgct tgaaaaatgg tgataatagt aacatgatgt |
| 15961 | catcctagct tggggggaat gataccatac aaaaataacg tgtcaacatt aagaatggag |
| 16021 | tttgagtctt taaagaccca cctattagct tctaaaacaa tgtaaggcca tgtacacata |
| 16081 | tctaagaatt ggtgagtatg tcaataattg ctcaatttta taacggtgcc ataatcccat |
| 16141 | actattacac tccatccatt caacaaatat taccaagtgc ttactacagg ccaggtacta |
| 16201 | tgctaagcag tgagaataga cagaggaggc agaataacat agtgaataag atcatgaggc |
| 16261 | caggtgtggt ggctcatgcc tgtaatccca gcagtttggg agactgacac aggtggatca |
| 16321 | cttgatccca tgagttcaag atcagcttgg gcaacatgac aaaaccttgt ccctactaat |
| 16381 | aatacaaaaa taattagaca tgcattgtgg cacgcacctg tagtcccagc tacttgggag |
| 16441 | gctgaggtga gaggatcacc tgagctcagg aagttgaggc tgcagtgagc cgagatcatg |
| 16501 | ccactgcctt ccagcctggg tgatgggagt gagaccctgt ctcaaacaac aataacagca |
| 16561 | acaacaaaca aaaaaccaaa aacaaacaaa aaagatcatg gactttgaac ttcaacagac |
| 16621 | ttgagttggc atcttggttc tccacccact acccatgatg tttatactca acttccctaa |
| 16681 | gcctcagtgt cctcaattgt aaaatgggag tagtaatagt agctacctct tagagcagtg |
| 16741 | aggattgaaa aagaaaatac gtgtaaggca cttagcacaa tatctgtcac atagtaagaa |
| 16801 | ttgagtttat tattattatt atcacagatg aatagacctc atgaagctcg ccgttcattt |
| 16861 | cctcgggacc cagcctatta ggaaacatta gcaaaagttt ttgattcagc attatatggg |
| 16921 | tcattcttga tatgtttttt cctgtcaaat cctcagtttt ctattaaaaa agaatttttt |
| 16981 | tttttttttg agacagggtc ttgccccatt gcctgggctg gagtacagta gcacagtcat |
| 17041 | agctcactgc gacctccatc tccctgtgtc aagcgattct tccccctcag cctcatgagt |
| 17101 | agctggaact acaggtgtgc accaccacac ctggctaatt ttctttttct ttgttgaatt |
| 17161 | ttagtagagg caatgtcttg ctatgttgcc caggttagtc tcgaactcct gagttcaagt |
| 17221 | gatcctctca ctttggcctc ccaaagttct gggattaaag gagtgagcca ccatgtccag |
| 17281 | cccatatttt aaaattaaaa ttaaaatttt aagattttat tttggtagag acaggggttt |
| 17341 | tgcaatgtcg cccaggctgg tcttgaaccc ctggcctcaa gcagtcatct tactgcatgc |
| 17401 | ctcctaatgt gttgggatta taggtgtggg ccactgcacc tggccaatcc ttagttcttt |
| 17461 | ttgagacctc tgactgcctt attttgacag gctctgtaac ttttgcagct cctggcacat |
| 17521 | cttgctttta cttactttta cattttctcc atcaaatgtt cactcattta ttccttcatt |
| 17581 | ctcattcatt cagtttatct actcaataaa cacatattga atacttacca tgtgccaggc |
| 17641 | actgttcact ccctatacaa acccagtgac tcattaacct accaagacat ctcttactga |
| 17701 | gtattctcac ctcactgggc aaacattgct aggccttcat ctacacagat cgcaggaggt |
| 17761 | ccaaactaat tgttgacctg ggtccatggc catttcagga cccctgcaga acaggagaaa |
| 17821 | tagttcttct ggtctcatag gattatgttt aagaagaaac tttctgcaaa tgatttcact |
| 17881 | ttgacaggga caaaagtgag caggaaacac tagaccaagg tcaaaaatag tttttaaacc |
| 17941 | aagtgcagtc attatgtaac ttacttcctg ttgaatattt ttcttgaaat ctcagtttag |
| 18001 | catacagcat agaattttgc aagaaactcc tcacaggcca cccaagttgg aaaaggtatt |
| 18061 | tcagtcatcc ctatatggtg gtgaatataa gtttgctctt ctgaaaaaaa aaaaaaatca |
| 18121 | gcaggtctga aaacgattgc atagggttaa atgagccgct tagtctctga acagagaatt |
| 18181 | ttgcttatga cattaccatt gcaattatac atgaaagaat ccttcaataa atgcagagta |
| 18241 | gtagaggatt ctcccagaaa agactgtaag attgttgtaa aatactttac ctggagtgtt |
| 18301 | aacttatctt tgttctaagg ttgcactttg gtttgtcctg ccctctcccc caaaaaggtg |
| 18361 | ctgacaacta gaagtaacaa atgcaaagaa ataggggaga aaatattaga agaaaaagaa |
| 18421 | atcagttaat ttacaaatgt cagagatact ggatgtagga ggcagaaggg agaatgacag |
| 18481 | ggtattggca gcaatttaga attagtcaat acccttgatg ctaccatgta gctactcagt |
| 18541 | tatctaagta aatcccagtc cacttagggg ttgatttccc accactcaat ttgaatggtt |
| 18601 | tgtctcagta gacaagggtg agagctttga aagccaccac taagcttgat tagatttgtt |
| 18661 | actttgtacc tatttcagta tgtaaaattt atacattgta ttaaagtaac actttaagga |
| 18721 | ttgatctata catgtataca caaaagatat ggaaggaaat gcctgacagc aaaaggtggt |
| 18781 | cgtgaagcat ttgtgatatt cgtagagata aaataaacaa aaataaattc agttccaata |
| 18841 | gtaaactgat caattttctc atacacatta ttgggcataa ttttttctta cggtttcata |
| 18901 | aacacaggta atcctgggca agaacaaaga aattatctaa atgaaattgt cctaaaattt |
| 18961 | tccaaacaca gcaggaaata taataaaata ttatctatta ccaaaaagtt cattatcata |
| 19021 | atttgaatta tttggaatac taagctaaaa tacagaatta tatgtgtatc catactatta |
| 19081 | acatacttta tggtatttta acagtaaact aaattagaag tactgttcag tgtttttgta |
| 19141 | gagtctaatt tctatgtcgt taaatataat cttatattat tatataatat aatcttatat |
| 19201 | tattatataa tcttatatta ttatattata taatcttata ttatataatc ttatcttata |
| 19261 | taatcttata ttatatgctc ttatataata taatcttatt atataatctt atcttatata |
| 19321 | atataatctt attatataat ataatcttat tatataatat aatcttatct tattatataa |
| 19381 | tataatctta ttatataata taatcttatc ttattatata atataatctt attatataat |
| 19441 | atattcttat cttattatat aatcttatct tattatataa tataatctta tcttattata |
| 19501 | taatataatc ttatcttatt atataatata atcttatctt attatataat ataatcttat |
| 19561 | cttattatat aatataatct tataatataa tcttaactta ttatataata taatcttatt |
| 19621 | atataatctt atcttattat ataatataat cttatcttat tatataatat aatcttatat |
| 19681 | tattatataa tcttatctta ttatataatg ttatattatt atataatctt atcttattat |
| 19741 | ataatgttat attattatat aatcttatct tattatataa tcttatatta ttatataagt |
| 19801 | ataatctttt attatattat tacataatat ataagtataa tcttatatta ttatataata |
| 19861 | ttattattat acaataataa tgtaaactga ctattgcagt ttacatggtg agttttattt |
| 19921 | ctgcacagat taaactttct atcatagagc tcttcttaaa agattctgaa tgactactca |
| 19981 | atatgtaggc aacttattta catactcaga taccttgcat gatgcaattc tgtaatataa |
| 20041 | actgattttg gtttttgaac ctcgtgttac atttctaatc caccagacat actttttatt |
| 20101 | caaaggtcta attgattctc atggattaaa ctggaatcct gtagtgatta tttttgtaag |
| 20161 | gtaaaaatgt ttgtgtgatt gggataatga ttccaaatgt tttgtttttc aagttttaat |
| 20221 | ttttgcatct tgagttgaaa agtacacctt taatgaatag acaattcatc aaaatgttat |
| 20281 | acctagctcc tgaattaatt agggtcattc atcagaaaac tgattatgta aaggaccaaa |
| 20341 | cagcaatgat aaccttcaag cctctgggat aggtagggct gtttgctcct gtgctaaatg |
| 20401 | ccctaagttc ttgttatcgt gtctgcbggt ggtagttatc ccattttctt tttcactctg |
| 20461 | ctgtccctgg catcatcttg tttctgtcag tgtaaatgcc tttagcaatt ttcagtgact |
| 20521 | ctgaaaacca gacaaagtga atttgttagt actggatttc aagaaagagc aaaagtaccc |
| 20581 | tctgagtgaa agttcaagag tcttcctaca agttaaatgc ctgaactcta gtccaagtgt |
| 20641 | ttggtgggag gtattttgca tgcataatag cttctccccc atttccttac tcttcttgaa |
| 20701 | tgtgttccag agagctgagg ttgctggcaa agtgactcct agattactta aggttcatta |
| 20761 | agtacttaaa gttcattaag ctcatacata attgtcacca gattatcatt ataattattt |
| 20821 | taaaagtagt ttatctgtga aaataagtaa aatacatttt ataaatgtta taaactataa |
| 20881 | gatgctccat tattttatct tcctataaaa tatgtctata aaaaagctca tttaaagaaa |
| 20941 | agttaccttc actgtgagat tgtctcaaag aaatgtctca aagatacctt cttaaatcct |
| 21001 | gccttctaca ggactgaagt taatataaca aagatattag tgttcttcct tgggattctt |
| 21061 | ccttgctaat ttccttgaaa acacccttga ttcccagaag tctcctattt acaaaacaaa |
| 21121 | actagttttt cttgaattga ttcatctgtc tacttaaaga ttttttttgg ccagttgtgg |
| 21181 | tagctcatgc ctgtaacccc aacactttga gaggttgggc aggaggattt cttgaggcca |
| 21241 | ggagttcaag accagtctgg gcaacaaagt gagacaccat cccctgcccc cacctcctcc |
| 21301 | cgccccaatc tctacaaaaa ataaaatagc ctatttattg tactctctac aataaataaa |
| 21361 | ataaatatag atgcctgcct gtagctccag ctactctgga gggtgatgtg gaaggattgc |
| 21421 | ttgagctcag gagttcgagg ccactgtact ccagcctggg caagagagct aaaaaaaaaa |
| 21481 | aaaaaaaaaa aaaaaatttt ttttatgtta tcttagttca agtttgagaa ataaaattaa |
| 21541 | gtaccaaatt tattcccaaa aatgtatgct tgtcaggaga tatacctaat gttaaatgac |
| 21601 | gagttaatgg gtgcagtaca ccaacatggc acacgtatac atatgtaaca aacctgcacg |
| 21661 | ttgtgcacat gtaccctaaa acttaaagta taatttaaaa acaaaaaaca gaaaaaaact |
| 21721 | acagattcaa ttcagcttta ggaattaaaa aaaagatttg atgtaattat atcattataa |
| 21781 | aatcaaattt tttaaataaa acatgaaatt gttttaattt aaaaaaaagt atgcttggcc |
| 21841 | tatatgtcaa ttcaatagct tttctttgcc agaaagaatg aagtcagtct acctgtatcc |
| 21901 | acatatgaag tattttagga ttttttgtaa ttttttgtta ttatctatat cattcaggtt |
| 21961 | taggaaagaa ctttacctaa aatactggct aattgtgccc caaaaaaaag aaagctagaa |
| 22021 | agaaagaaca cacacacaca cacatacaca catatacaca cacaaaaaaa acaaccaact |
| 22081 | gatcaactgg gagtttcagg tttatcagcc tttagtctca atgctgtatc actgtagtaa |
| 22141 | aaatcaggct acttgatagc acctagaaac ccattttaaa atataaactt gtaaagcatt |
| 22201 | tctgtaagta aaagttagta tgtttataat gacatttttt cttttaagac atctttatta |
| 22261 | ttatcataaa aaatacagag aagcaaaaat aagaatgtag aaatcaccca gtttttcctt |
| 22321 | tccaagtaaa caatcataaa aaacttggtg tatatatata ttgcagcaat tgtatgtatg |
| 22381 | taatggtctc tgtatcaacc aacacatttt tatcatttta ctgaaacttt tgagtgttgt |
| 22441 | ttgatgctga tatcatggtt tttcatgtgg gaagattcat atgagaattg attgtgtgtg |
| 22501 | tgtgcgtgtg tgtgtgctcg tgtgcgtgta gcagcaggaa gtatataaaa ttattttctt |
| 22561 | tttatagacg ctggttgaga tttttcaaaa tattggtgat accaatcttg ctgatttcac |
| 22621 | tgctggattg ctcaccattg tcgtctgtat ggcagttaag gaattaaatg atcggtttag |
| 22681 | acacaaaatc ccagtcccta ttcctataga agtaattgtg gtaagtagaa tatgtagtta |
| 22741 | gaaagttcag cattatttgg ttgacaaaca aggaattatt aaaaccaatg gagtttttaa |
| 22801 | catcttttgt tttatttcag acgataattg ctactgccat ttcatatgga gccaacctgg |
| 22861 | aaaaaaatta caatgctggc attgttaaat ccatcccaag ggggtgagtg tggtgttcct |
| 22921 | cttagtacta atacattaag tcagtaagtc agtctttttt atttaaataa aaccttttat |
| 22981 | tacaagcttc atttcactga tactccttca atagtcctat ttgtgtgtga tctggaagaa |
| 23041 | acaaccataa gacaaacagt atgtgtgtag ataaacgtca agccatttgt ttaaaagcag |
| 23101 | cccgtatatg ctcttttttc ttcattttct taaaacatgg cagaacatgt acggttaaca |
| 23161 | ccagagtcct gatttaaact tatacataac attataatat aatataaata gaatcatttc |
| 23221 | tcagtaacat ctattgcact gtgaacaatc tgcttaagga atcagggacg tctggcctaa |
| 23281 | attctctatc atttagttta atcaattttg gataataatt atatcctcgt acccataagt |
| 23341 | aaaatgcatt atcttcaagg gaagttttag aaacatttgg tagtttggtg aattagaaga |
| 23401 | gtaaaagatc aaggtccttc attcatctct gtcaccttcc agttttgtga cctgaggaga |
| 23461 | ttctcctata taagacaagg ataatgaaaa cagccgaagc ctcaactcaa gcttattatg |
| 23521 | aaacaccagt gagatctctc atgaaatgaa gagtagtcaa gacaccctac accataattt |
| 23581 | tctcatatgg ggttacaaag gaggcctaga cgtttattat atttgtaaca tacactcata |
| 23641 | acatttaaga ctagacttgc tgttaatcaa gaagtactag ttttccctaa gactggaaga |
| 23701 | gtttggtacc ggtgtttcta ccaggaaagg gagtggaggg gagggaaggg cacggagcca |
| 23761 | ctgtttggta ccagtgtttg ttttccatgc tcttaataga aacttcctca ggcaagctta |
| 23821 | ttcccagagc ttattctaca gggaactgct gtattcccat ttgatgaatc ttataatttc |
| 23881 | ctctaatatc tgttccaaac tatttgaaaa agcacagctg gctacatggg acttaatttg |
| 23941 | catgctatcc attacagtgt ttctcaaact tacccttttg taagaatcac atgggatcct |
| 24001 | ttttcacaca gattcctggt tcccacccca aacctattga atcaggcttt ccagaggtgg |
| 24061 | ggctcaggaa ttaatatttt tcaaaagtgc ctcaagtgat tcttgccctc aaggaaactg |
| 24121 | gagaaactct ttaagagagg atcaacaaat ctaatctaac aactagataa tttcattagt |
| 24181 | tagaaacaat tacagaaaaa gatactttac agatctcttt tgtcaggctt cctaggatgt |
| 24241 | atcaagttat aggtggtcca cagagcaggc cctgtcccat ccatccgtgt tccattagtt |
| 24301 | ggtttaggga actcatttct tctgccctaa attatccctc caggtcctct gcagagcttt |
| 24361 | cctgtgctct cctcctgaga acagatttct agcctctcgc ccttgagttt aatgaagctg |
| 24421 | aatcctttga cagaccacct ggtcagcttg gtcagctaat ttaacctgcc tggccccagg |
| 24481 | gactttaagc tactccatga gggcagtgac cccattggtg ttgttcagct ctgtcccaga |
| 24541 | gcacagagct catctcacag taagctctca gaaaacactt gttgaaggaa taaaataccc |
| 24601 | aagcctatgt cattataaac aagccacaac aaaggccttg gttatctgat ctcacttggt |
| 24661 | agcacagaag caattctgat gatattaaca aacctgaaaa agaaagggag tggagagatt |
| 24721 | ggaaaaaagc aagcagagtt ttaaagtggc cctgaggtga gcacagtgaa aggaagaggg |
| 24781 | acagagagat tgtgatatgc ccccttttga tctcctccct gcttcaatcc tccattaagg |
| 24841 | ctgctgctcg tagttccctt tatcctattg agggtccata agttaggatc actcctaacc |
| 24901 | taggaccata cgttaggacc ataagttagt ccccaataac aatgccccat tggacagcca |
| 24961 | aaaacagtgg ctccatgccc ttccctctac tccctttcct ggtagaaaca tacaactttt |
| 25021 | ccttgtccag tcaacaccta agcgccccac taggattcac tcattctccc atcctgggtg |
| 25081 | ctttcttaac cagcagacac tatgagggcc agtcatgtgc ctctcttctc atgcaactgg |
| 25141 | tccttatctg caactcgtaa tctgcccagt tcctcactca tcctgcccag gcttctcttt |
| 25201 | tacctcttct gttatagtgg tgtgtgtgtg tctgtgtgta tgtgtgtgtg tgtgctcacg |
| 25261 | cacgtgtatg tgtaaaccca taacaagatt gacagctctg tcctctaaat tttaatccct |
| 25321 | attcactctc ttgcctcctg tccttgccag ttccagaggt ccccttgttg tcatgtttgt |
| 25381 | tactctcttt tttcttttct tttctttaac agagtctcac tctgtcaccc agcctggaat |
| 25441 | gcagtggcat gatctctgtg gtgtgtgtgt gtgggtgtgt gtgtgtgtgt ataaacccat |
| 25501 | aacaagattg acggctctgt cctctaaatt ttaatcccta ttcactgtct tgcctcctgt |
| 25561 | cttcgtcagt tccagaggtc ctcttgttgt catgtttgtt actctttctt tcattctctt |
| 25621 | tctttcttcc cttccttccc tcccttccct ccctttccta ccttccctcc cttccctccc |
| 25681 | ttccctccct ttcctacctg cactcccttt cctccccttc ctaccttccc tccctttcct |
| 25741 | ccccttcctt ccttccttcc tctctctctt ttttctttct ttctttcttt ctttctttct |
| 25801 | ttctttcttt ctttctttct tttctttctt tccttctttc tttctttttc ttttcctttc |
| 25861 | tttccttttc tgaaacagaa cctcactctg tcacccagca tggaatgcag tggcatgatc |
| 25921 | tcggctcact gcaacctctg cctcccaggt tcaagcaatt ctcctgcctc agcctcctga |
| 25981 | gtagcbggga caacaggcac acactgccat gcctggctaa tttttagtag agacggggtt |
| 26041 | tcaccatgtt ggccaggctg gcctcaaact cctgacctca ggcgatctgc ctgcctcagc |
| 26101 | ctcccaaagt gcbgggatta caggcactgt tatagacccc ttttgtcagg tttcctagga |
| 26161 | tgtatcaagt tataggtggc ccgcagaaca tgccctgtcc catcaatctg ggttccattc |
| 26221 | attggtttgg ggaactcagg cctactgttg tttttaaagt atgtgctttg cagttgtact |
| 26281 | tgaaagtgtg gcacaggggc aaagccaaca aaaccacagg gtttttttcc tggctgcttt |
| 26341 | ctgagggcta ctcttctcct ctagctggag tggtggtgca tttgccaagt gttcagaccc |
| 26401 | agtctgaagt actctggtat tctgtttggt attctgttta tgacgctggc ctgtcgtttg |
| 26461 | tggaattttg ggcccttcag ggttcacttc tcccggtctt catcttttca gataagtgtg |
| 26521 | tgttcacatg ctcactccct tgggctcttc atagtgttgt cttatgatca ggcctgacat |
| 26581 | gcttggctgg ggccaaacag gcccactgag caaaaaattt ggagagttat atccacagaa |
| 26641 | ggagggcaag ggcaagggca aggacaaaag gcttacttac aggtcattag aagatatcca |
| 26701 | gacgggactt caattggttt ggggcaaata attggcacac ttagggttca gcaagacagc |
| 26761 | agacatgagt ggcaggagta tgacagactt agattctccc aatatatacc aagaacctac |
| 26821 | tatatgtgta gacactaatt acttacttac acaagtgatt cctcattgaa tcatatgaga |
| 26881 | agaatatata gtccccattt gacagctgag gaaactggga tgtggtgggt ttaaataaca |
| 26941 | tgcctaaagt catttgagag tgattctggt cctttgactt aagtctttgc actttacact |
| 27001 | ttgcacacct ggataatcaa ggatcaggtc atatttggag aactaggcag ccaggcaggc |
| 27061 | aaagtgcatt aggctgctat caaaactaag ctgtatgtga aactggaaag ttatcctgac |
| 27121 | aagtccgagt gacctgaagt agggttgact gggtctcagg agcaagtcag gagcctgttt |
| 27181 | tgagaagtgt ggtcccaagt cacatcccaa ctagcaggtg atgtcatgct gagtcgccca |
| 27241 | actgttggat cagggtttcc aacatcctgt ctgtctacag atggggttca ttccacagtc |
| 27301 | tcattgbggg gtaggatggg atataaggag aaaaggagtc tgaaaattaa cctcactggt |
| 27361 | agggagagta aggctacaag ggcagagaac cagacagatc ttgttaggta ggaaatcgtc |
| 27421 | taatatctaa catttagacc tggacccact aactgacatc aagcaaatca cttcacgttt |
| 27481 | ccaggtctaa gttcctttat caacaagaca agacaagagt ggtggattca acaataccta |
| 27541 | ggatgctttc tctatggttt tctctaagaa ccttatttct attttatttt taccactgtt |
| 27601 | ctctaattat aggggaaaat gtagtttcct tattaaagaa actttaggca taagtgatgt |
| 27661 | aggtcgggga gacctttcat ggcbgggggc ctctggaatt ataccatatg gatagtcatc |
| 27721 | aagatatagg tccatctccc aaattttcat atttgagaga acctcgtggt ataatgtgta |
| 27781 | ggttaatttt tttatatggt ttgtagggta gctctggttt tgtcaaacag tagtcattgg |
| 27841 | accccacaat cactgagata ttttagtcac tatggagcgt tgtgataatt acattaattt |
| 27901 | aatctttttt gccttaagaa gattttaaat gtcctggctc tcaaaggatt ttccactgag |
| 27961 | ctcttttagc ttcatacatt ctttgtctct ttaacagagc tgatacctct tcaaacacag |
| 28021 | taaatctcat attatggcta gaatctcttt taaattcaaa gaactttaag atggaattgt |
| 28081 | ggaaggagaa acactaacat gtgaaatggc atggatgggg ctgtatgata ttaaagaatc |
| 28141 | agtaacagca gatttattca tgtgagagat agagaagagt cagcatggct aggctcaaaa |
| 28201 | atagaggaca aagaaatcag ccagtaagat aacaccaact tctgattaaa cattataaaa |
| 28261 | gctgctacta tcatgtatgt atttcgtgtg ctttttgttc ttttggatca agtactttca |
| 28321 | tgtctaatat gtgactgagc agatatagca tttgatgaga tggggaaaaa ggatggtggt |
| 28381 | caaatcttca cagcattttt cacttaaaaa ctcactaggt ttttgcctcc tgaacttcca |
| 28441 | cctgtgagct tgttctcgga gatgctggct gcatcatttt ccatcgctgt ggtggcttat |
| 28501 | gctattgcag tgtcagtagg aaaagtatat gccaccaagt atgattacac catcgatggg |
| 28561 | aaccaggtat gggtgccctt ttgctgaact ggttttatag ggctggaaac aggaaaaaaa |
| 28621 | cataaatgga aaagattttg gtgtcagcta aagaaggggt tggattcttt caccagacct |
| 28681 | tattgggttg gttttcctct tgtgtttgct aattagaaat tattttggaa tagacacact |
| 28741 | gcatcacttt gcttttttaa gaaataagtt gaaataacac taattttaaa agagctaatt |
| 28801 | cagtcaggtc ataaataaca ttcaaagtga acatattagg tcagaaagag aattcttcat |
| 28861 | gtaacccagt acctcttggc aattattttt aacggcagtt ataatgtgac aaaacagtgc |
| 28921 | aacaaacatt aataatagtt gtgaccactt taaattgagg tggttcactg tgcagttaat |
| 28981 | atatacaggc aatttttgta ggcatgtgat acattgccag gtaagctgat ttcacactgc |
| 29041 | ttgcaaattt gcaagttctg tatctatatg tttctgaaaa tacagtatag caataacagt |
| 29101 | agttaaatgg ttagtaatca agcagaataa cagcactgct agtcataata tgcttcatag |
| 29161 | tgaatattca accacagaaa aacactttat gtgagagctg cagacattcc attcactagg |
| 29221 | cagtgaaaaa gcttagcaaa tgatcaggtg ctatttcttg tcaactttaa aaattatcga |
| 29281 | gagcaatgag acctctctca gatggtatgg cgtccaaact cctgatgtcg tacaaggacc |
| 29341 | ccaagtacct atcacggtaa aaattaaatt ggaccaccac gcagagtagg catgggagtt |
| 29401 | ttcattctta atgtacttcc tgaaatactc agcgaaggtc ttgcaaagat tcaatttgta |
| 29461 | ggatcgttgt catccagtct cttccttagg aattcattgc ctttgggatc agcaacatct |
| 29521 | tctcaggatt cttctcttgt tttgtggcca ccactgctct ttcccgcacg gccgtccagg |
| 29581 | agagcactgg aggaaagaca caggtaggaa caacagcctt atgatatcca tctcagagaa |
| 29641 | caagtcgagg aatggcaaca gaggaaggct cgcaccgagc ttagcaggac aatttgcctt |
| 29701 | tcagacttgt acttcctaat ctgattcacc tcaggcctat tcctcttgtt ccactccctc |
| 29761 | acctgaaatc tcttaaaaaa caacatgtat ggttttctga tacagtgatt ctcaaatcta |
| 29821 | tttgtcagtg cttacctgtc atgtagctac acttacctgc tgtggtcaat aacaactgac |
| 29881 | aaggaatcag taatgaagga tgctgatttt tgttaatttg ttacctgggg agacagatgg |
| 29941 | cctggtaaaa gcgcttcctg gctttaggag ctattctatt ttcaggaaag tgaaaagctc |
| 30001 | tattcacatt tcctttagaa ggatcaggaa cttcctccag gggcgtttgg tcccacagaa |
| 30061 | caacagtttg ctagccaaga atatttttgt tgtaacattt ttggcatttc ttcctgagta |
| 30121 | agatttatgt tgttgaagtt ctgcatgtgg gtttaatctt atcttttaaa tgtggtttgc |
| 30181 | agttgttgcc tttacaaggt ggccaaaaag ccagcatcct agttaatctc tggataatcc |
| 30241 | ctccttcagt gttcagaaag ctcaggagcc atactcaaag gccactcttt ccagcaggac |
| 30301 | acagtcgaag agaccacacc aagcagatgg gcacaagaga cagactcagg aattttgctt |
| 30361 | ctgtctctta tgccttgagg tccttatctt cccaacacag aaagaactat tatttaggag |
| 30421 | taagaagtgc attgagactc caaagaacaa caaacccaaa ctacatagtg caatacacac |
| 30481 | acacacacac acacacacac acacacacat gcacacacac atgcacagtc ttcacagtct |
| 30541 | tcaaatgggt tttactaagg cggggacact taaaaaataa atataagtcg ggcacagtgg |
| 30601 | ctcatgcttg taatcccagc actttgggag gctgaggtgg gcggatcaat tgaggtcagg |
| 30661 | agttcgagac taacatggcc aacatggtga aaccccttct ctactaaaaa tgcaaaaatt |
| 30721 | agccaggtgt ggtggtagat gcctgtaatc ccagctactt gggaggctga ggcaggagaa |
| 30781 | tcacttgaac ctgggaggtg gaggttgcag tgagccaata tcgtgccact acattccagc |
| 30841 | ctgggcgaca agagctagac tctgtgtcaa atatatatat acacacacac acacacacac |
| 30901 | acacacacac aaacatatat atatatatat attctcatat atatatacga gaataactga |
| 30961 | aaaaactaat gactccaaca gtgacttgcc cagggagggc tttaattgtc cccaatatgc |
| 31021 | atgtgatgct cctttcctat ttataatgga tcttgtgaca gacttggaag aattggcctc |
| 31081 | ttccagtttt ttggctgctt ttcaacaaaa aattttattt tcagaatttt tcttaatttt |
| 31141 | ttattttttt tgcaaatatt tccagctctg agggagcagg taaaataata cagctcatat |
| 31201 | ccggaggcat agccagagat ctgtggtcaa ggggaagagg tcagagccaa tttccaaagc |
| 31261 | tgtgcacatt tcaggctgca gcbgtcagca gcaggaactt ctgctgcttt catttgagac |
| 31321 | ggagcactga caagctctcc agagcacatg gtcttcagcg ggtgcaggca aatctcaaac |
| 31381 | tgctgatgtc cattctcctg atgttacctc caggctcaaa tgaggcatgg gctggctgtc |
| 31441 | ccagtaagta tgcccatgta aagtgacctc cttggcacag ggtttggggg ataatggtgc |
| 31501 | tctggtaagc taataccccc tgccacatac tggcatgtat tatatttagg aatgaaggat |
| 31561 | gccaggattt cagcattaaa gaatgaaacg agtaaagaaa tggcttcatg tttaattatt |
| 31621 | gaaatgtata ctaaattctg gggttaaaag tctaagtgcc tgaactgaaa tccagggact |
| 31681 | gatggacaaa taaactggca gggcttctct tgtgtggctt ctcgtgtttt ctcttatgtt |
| 31741 | ggaacttcag tttcagcatc tacaaaatgg aaatgataat tgcatccatt tcacagggat |
| 31801 | attgtgagga ttaggcaaga aaatgtaaag ccttagcaca tacttgtaaa tgctcattgt |
| 31861 | ttcactcctt taggcaagaa tagcatctta gcatctatga tacatatatt gtgcctggca |
| 31921 | cataagaagc acctggcata tatttgctga atgaatgaat ggaagaatga gtattggtgg |
| 31981 | acacgaactg tttttagcac atctacagat tgtagaagaa ttaaacattg gaaccttttc |
| 32041 | tttccctcac ccccatctga cttccactga ctcacagaaa agtaattctg attacaaacc |
| 32101 | actctttagc tctgattacc ccctccctta acagaataat tttttagtaa ctgaatctgg |
| 32161 | ttgtagatat aaagtctaca gagtttctca caaacaagcc ttatcaagta agtagaaata |
| 32221 | attgatcact caccaatttt aatatagtgg ccaaaatgag ccttggcctc ccataattgg |
| 32281 | agactcatta tgacatataa ttggcaagta ggtggccact ctcctggcat gagtttggca |
| 32341 | gctgactgtg gtccttgtcc ccaaatctcc atatctttct ccagttcaca gagctcccca |
| 32401 | tccacagtga gatgcaagtg gctcggtaaa tactggttga gtaattgcat gcaaggaagg |
| 32461 | aagggaggga taagggaagg aaagaaagaa gaaaatggaa ggaaggacaa gggagaggaa |
| 32521 | gggaagggat atgaaggaaa gcgaagagaa gggagggaag taggatatca gcttctctgc |
| 32581 | ttagtcagga gaagggaaag agcaccaggg tactgggaat ctaggatggg gataggaaga |
| 32641 | gcatagaaaa gccagccaga catacaaagc acgcttttta gtacctcata cacatcatat |
| 32701 | tgcctcaggc tgacccaact ataatctaaa tataaatgat caatttgcca ggtagagcaa |
| 32761 | gtgctttcta attttctgtt tatatttgct gtgtctgtct cccgtaactc agaccttttg |
| 32821 | cactagacag tacttgtgct caacaaaatc ttcccagttg tttcctctct gattgctttg |
| 32881 | cttgtagttc cctgtctcct tttcttcttt gtttcagagc ttctaatctt catcttggct |
| 32941 | ctgccccagc tgtctcaaca gagaattcat tggtcatcta atcaaacgag agagtagtcc |
| 33001 | ccaccccctc agcctctctc ctaactagta tggatcttag gagagctatt tttgccagcg |
| 33061 | aaggtaaagt ttctgaagct tccttttacc cctgctattg ccccgttact ccatagtcac |
| 33121 | tgtttcaggc tgccttgggg caaagctctg agcctgcctc cgtggaactg tcagaggagg |
| 33181 | caggagacac aatcccacct ctcaatgtgc aaatgtggca tttcgaattt gcaataggat |
| 33241 | gagtaaattt attttctctg agactttaat gagaccatgt gctacaagta cgaagtgtta |
| 33301 | tcagagttgc tattatagct gcagcttcaa taatgcagaa aaatcagcta tgtgccatga |
| 33361 | gaatttattt cccaagaata cgacaatgat ctctgttgtt gggaaagggg ggattggtcc |
| 33421 | atgtttcctg ccatggtaaa taactagaag cttggcctga atggacgccg aaaccgcagg |
| 33481 | tgtgtactat caccaaataa aatccctgtc aaactggctt taatttcata ggatcagtcc |
| 33541 | ccatattttc tttaacttct ctgccatagc atatatagat gccatttttg ttcagttttg |
| 33601 | tggcttgagc aaataactat cacttttctc gacagtattg agcagaaggg ggagacaggg |
| 33661 | aagtatgaag tgtgtctgtg aacaggctgt ctcatacaca catccagtga gctggaagac |
| 33721 | acaagggaga aggacgaatc cttttcatag gaggtgtgtg tcttccaggt tgctggcatc |
| 33781 | atctctgctg cgattgtgat gatcgccatt cttgccctgg ggaagcttct ggaacccttg |
| 33841 | cagaaggtat aaccctgctt ctctgcatac cgattgcata atttcccttc actactctgc |
| 33901 | taccagataa ataacaggag atttaacaat catcacatgg aaaaccattc cctgaataac |
| 33961 | acagccttct ctgtctctct tggcagtcgg tcttggcagc tgttgtaatt gccaacctga |
| 34021 | aagggatgtt tatgcagctg tgtgacattc ctcgtctgtg gagacagaat aagattgatg |
| 34081 | ctgtaagtca cctaccacct atatttatct gaaataagat ttggttctta tatgcttcct |
| 34141 | gccatatcac tatattcccc ccatccccta agtctcactt gtgctttggg aactccagag |
| 34201 | gagaaattag aattgbgggg ataaatcaaa gccatgaagt cttccatgtg aactgcattt |
| 34261 | tgtgaagatt ctgtatctct gggaaggtca atttccagca agaatcccat atgaggctca |
| 34321 | ttcatacacc tttggccttc taaacacaaa aagcaaaaaa atgtttaaac accctagttt |
| 34381 | agaaggacaa acaacataca aaacaatgca aaaacaaaat acaaaatgta aaaacccacg |
| 34441 | ctgactttaa ctctgactaa cataaccttt gcaactattc tgagaaacta aaaatcagaa |
| 34501 | gctaaatcct tcagagttcc tctctaattt aaaccataaa aagaatgaga agacttccta |
| 34561 | catattgata tggaaaattt tccaagacaa atcgttaagt gaaaaagaga ccagaataga |
| 34621 | aatgctgggg aaaagcacag acacaggcag aaataaaagc ggatacaagg agggagggaa |
| 34681 | gttgggaatg ggtggagaag ggggagggtt gtgagcaaga gttttcccct ttttaatgat |
| 34741 | aattccctta tgaaccatac aaatatatta gctattcaaa aatttagaaa taaataaaat |
| 34801 | gggacagttg gtcttaattg agaagcccag tttctacagc tacttaaata taaaatgaat |
| 34861 | gaagtcacaa tatttttaaa aagctgtgat ttggccaggc acagtggctc aggcctgtaa |
| 34921 | tcccagcaat ttgggaggca ggaggatcac ttgagctcag gaatttgaga ccagcctgcg |
| 34981 | taacacagtg agaccttgtc tctactacaa ataaaagatt aaaaattagc cgggtttggt |
| 35041 | ggtatgtact tgtgggtagt cccagctact tgggaggctg aggcgggagg atcacttgaa |
| 35101 | tccagaagat ggaggctgca gtgaactatc atggtgccac tgcactccag cctgggcaat |
| 35161 | agagtgtgac cctatctcaa aagaaaaaaa aaaatgtaat ttgtttgtgg atcattgatc |
| 35221 | ttatttttat aggtagttat cacatgatgg tacctgatac attaatataa ttcttttcat |
| 35281 | ttctattttt ttccctaggt tatctgggtg tttacgtgta tagtgtccat cattctgggg |
| 35341 | ctggatctcg gtttactagc tggccttata tttggactgt tgactgtggt cctgagagtt |
| 35401 | cagttgtgag taacgtaaaa cccagatttc ctataaacag aacaacacac tctgagcttc |
| 35461 | cttataccat tttgataaat atagtgaagc cactttcttt cgttatagtt actgtatatt |
| 35521 | gagtgcttct atgcattaag cagacagtgt tttacagaca tacttaatct tcaaaaatag |
| 35581 | cctatgaata gttttgatta gtctcattct acaggtgaga aaagagaaat tcagaaaagc |
| 35641 | taagaagcat ccctaagatc acacagctag tacgtggcag agctaagatt tgaacctatg |
| 35701 | tacctactca tgtcctctac tgcbgtgctc tccttttagt tgtggtaaga ctaaaaagta |
| 35761 | catagtttag ccaaaggctg aagttgcttt gtagtttctt ctttaaatga gatgaggaat |
| 35821 | gtaaggcgct cggccccagg cttgggacaa gacagcbgtt atatattata gttcattttc |
| 35881 | ttttttaaag ctagtaacca aaagttacat aacccagaac aggaggactt taaaatagct |
| 35941 | gatgacagcc aaaattcatt caaatctctc atataaccaa taaggcatat aaccaataaa |
| 36001 | ccacacaatt atgtgaagga aacaattagc tcatatgcag agtgaaggca gcaaagacat |
| 36061 | gatttaaata atggaaaaca gcctgtgagg gaaggtgagg agcctgaagg tggttttttg |
| 36121 | gttggttttg ttttgattct agagaggcct tagttaagtg ccctcctggg cctagagggg |
| 36181 | aacagagtct ttctctccct cctcaatgtg tccatcgtct ttccagactt ctggcccctg |
| 36241 | ctctgcctgt gcttgggccg tgaaagaaga agacacctgg aaaaacatca agatagatgt |
| 36301 | cttcagctta gcatccgctc acctctcctg tcccaccagc cagttctcct tcctgccctc |
| 36361 | cttccctacc ttggcatatt ggtgcctacc caaaaggctt gtgttttcta cctcagcatc |
| 36421 | ctaaatgatt gcccaagggc ctgagaaggt ttgcaggctc ccaaagttct caatttgctt |
| 36481 | ctaggacacg tctcagaaac gctccttaga agatgcbgtc tctcatgctg tatatgcctc |
| 36541 | agtgtctatt ttttcttgtg tggatgtggt tggaaaagaa gggttttaca gagagagatt |
| 36601 | agcttataaa cttttaagtt aaaaatcagc acaaacctgt aagccaagga ataactggtg |
| 36661 | gcacatttta gctgctagtt tggtatctag gcttttagcc aacaaaaagc agtatcctcc |
| 36721 | acagtattta atttccagta attatcatgc tgatccagag agcacaatgc tgaaagtaca |
| 36781 | ctaaacagat actctttctt caggaattgt atttagcatg tctctagtca cacatgaaga |
| 36841 | gtcacggaat ttatctgatt gcatcaggaa caagaaatca atagtctggt tgacaggaag |
| 36901 | gacttatttc tggtcctcca gtctcctatt tctataaaaa atgactaagt cacaaagaag |
| 36961 | cgccaggcac tggaagaccc aggggttaaa gacagaggct ctgctgccct ttacctccag |
| 37021 | gccttttctc acttctctta agaagtttag ttaacaataa aaatcaggat aatactgatg |
| 37081 | acctttcagg gttatggcag ggcttaaata agatttcaca tgcgtgaaag ctctatgctt |
| 37141 | atcctataac gtgttgtgca cgtgcaagta tgatgctcat tatttctctc agccatcaga |
| 37201 | agagaggcac agttctcccc tcccctgccg attccacaca aacaccagct gttcatttca |
| 37261 | gagttagcta caggaaaatg tcatctgcaa taaagacaga gtccaaaaca ccagaatgat |
| 37321 | gggctcttta gtagcbgttg tttttaactt tttattccaa aatacggctg ttccaaaaaa |
| 37381 | tcttgacctt gatatttttt cttctagtcc ttcttggaat ggccttggaa gcatccctag |
| 37441 | cacagatatc tacaaaagta ccaagaatta caaaaacgta agtacctttg tgagacattt |
| 37501 | gctggacttg ggtttactag cctgaagttt cagcagctcc attttacgta caaggtagcc |
| 37561 | aaagggagaa aatgcctatt gggaaagtct gttagtccac agggagtgtc atgaaaactt |
| 37621 | ttgatccagt gcaccttctg acacccatgg cttatgtgaa ttttgtctat gctagctgaa |
| 37681 | tgtctttttt tttttttctt tttagatgga gtctcactct tcacccaggc tggagtgcag |
| 37741 | aggcaggatc tcagctcact gcaacctctg cctcctgggt tcaagtgatt ctcttgtctc |
| 37801 | agcctccgag tggctgggat tacaggcagg tgccaccacg cctggctaat ttttgtattt |
| 37861 | ttagtagaga tggggttgca ccatgttgcc caggctggtc tcaaactcct gagctcaagt |
| 37921 | gatttgcccg tcttggcctc ccaaagagtt gggattacag gcgtgagcca ctgcacctgg |
| 37981 | ctagctgaat gtcattttaa tataaaactg ttgtaagagg aatttaaaaa taataggcaa |
| 38041 | tttctagatg ttcatccaaa attttgttta tctggtatat aattcttatc ttctgtaaga |
| 38101 | ttttctttat tcagttctaa caagagatta tcaagttctc tatgaccaag accttattat |
| 38161 | ttgacacttt gctttatcct tggtattaga agaaataaaa tatgacacag gaaatgaaaa |
| 38221 | acaaagaaat taaacatccc attgatggac ttgtgtcacc tgcaaataaa attgcttagt |
| 38281 | ggtgactagg ggaaacaaag ataaaaaagc agagaaagta aatcaagaaa atacaaattt |
| 38341 | agcaaataat aataataata atatcaaccc taggaagttg tagtctaatt ttaccttaac |
| 38401 | attaaaaaaa gttggacctt gaaactggga tttcagtagc tgctacttaa tttatttccg |
| 38461 | tgatctttct gttatgttga gccaagtgcc attaagagct aaaatgattt agagattttt |
| 38521 | ttggctttgg ggagggagct ctaagctgag ttagggaatt ctgacttcta ggctccatct |
| 38581 | tggtgagact ttgcccaagt cattcagcct ctcaggacct cagttttctc atctgaagca |
| 38641 | gctagactaa aattgctgat tcctgaagac tagaaaagtc tatgattctg tgattcctgg |
| 38701 | gttctatttt gggtgtggcc attgtatgtc agggtgagaa cagatgatat cataggcctc |
| 38761 | agttctgcaa atcccattga ttttgctttt tttctttgct acttttaaag aatgttagca |
| 38821 | aaatcaggag ggtcagtggt atgaacttca ggcaaatcat ggcactgttc taagcctcag |
| 38881 | tttcttttct ggaaatcaga gggttgaata aattgctttc taaggccctt tctaactttg |
| 38941 | accttctatg agactgtaga ggtccaagag tttttgtaac ctgttgtctc cctattcaag |
| 39001 | aaaagttgta gaggccgggc acggtggttc acacctgtaa tcccagcact ttgggaggct |
| 39061 | gaggcgggtg aatcacttga ggtcaggagt ttgagaccaa cctggccaat gtggcaaaac |
| 39121 | tctgtctcta ctaaaaatgg aaaaattagc tgggtgtggt ggcagcacct gtaatcccaa |
| 39181 | ctacttggga ggctgaggca ggagaatccc ttgaacccag gaggtggagg ttgcagtgag |
| 39241 | ccgacattgt gccactgtac tccagcctgc gcaacagagt gaaactccat ctcaaaaaaa |
| 39301 | aaaaaaaaag aaaagaaaga aaagttgagt gctgctaccc agctcctctg agcaactgtg |
| 39361 | acttgactcc ttgctaagta gccagaaatg taattaaata cttgaggctt gaaattattt |
| 39421 | aatcccagac aatttctttt aatgccagat tgaagaacct caaggagtga agattcttag |
| 39481 | attttccagt cctattttct atggcaatgt cgatggtttt aaaaaatgta tcaagtccac |
| 39541 | agtaagtatt ttatccctag aaatttgttt tctaacctct tttgagactt cattcattct |
| 39601 | acaagtattt actggggtcc aatcaggaat aggccctaga ccctcttccc tttgtgtagg |
| 39661 | gcaatgagaa ttaaaatata acatccttgc cttcaaataa tttacagtct atttggggat |
| 39721 | taaaaaaaca catatgttaa aacccaggta gtaataacta tgccagacaa aaacattaga |
| 39781 | gatgtttcta ggcagaattt gagtaagttt caaataagta gtatagacag ttgagagttc |
| 39841 | atagaaggaa gagatccagg ttagttggac taacaggctt tgtgcggcag ttggctgaag |
| 39901 | gaaagaaatc atgtcaagtg tttgagaact atgtgagctg agagacagtg gaaacatgta |
| 39961 | ttggaggtag ggttgcccag ttgcagcaaa aagtaaaata tactagtgga ataaataagt |
| 40021 | agaaaatcct ggcaccaaat gccatgctat gctttgaact tggagtttgc ctctaaatca |
| 40081 | gtgtttttct aagtgagttc cagggcaaac tgattcagag gaatgttcac agattttgca |
| 40141 | ataacaaaag ggctctgtga taccaaggga atttggaaaa cccagactta aactaaagag |
| 40201 | ggtattttgg ggtgtgtgtg tgtttctgtt ttgtctttta ctgtcttgga gcctttgata |
| 40261 | tgctactgtc ttctcagagc ttctgggtag gagtagggta gcctgggagt agacagagat |
| 40321 | ctactccatc agaccttaca atttcttttt tggcaggata gctcaaggaa ttataccctt |
| 40381 | tgagaaatag cctttccaga taacagttgc cattaataag ctttaggtgc caggcatttt |
| 40441 | aagtaacttg acatttattt ccaaaggttg gatttgatgc cattagagta tataataaga |
| 40501 | ggctgaaagc gctgaggaaa atacagaaac taataaaaag tggacaatta agagcaacaa |
| 40561 | aggtgagatg acatctttct tttccccctt aaattatttc ctttccctga tgagagcagt |
| 40621 | tagagggtct aaaattaaat ctatcctctt tagtatccag atgtgaatga acaaatgaca |
| 40681 | tgtacgtatc aaagaacaac tgagctattc ttataggcaa gcgggagtgg aagggaatga |
| 40741 | aataaaatca gcagcgctgc ctggaaacac aggatgtgat ttttttcccc accatgaaca |
| 40801 | gtgtctgcat tattttctat actttatttt tactgaaagg aacaaaataa gtgcaaattc |
| 40861 | aatgccagaa aactacccac catagaaggc agtctaagtc cataaacttt tggagctgtt |
| 40921 | aacttttact cagattcttc tggccagtgt atttcttggc aaagttccac aatcatccag |
| 40981 | aaaacaaaag tttcctggct cctctgttct cccagttttc ttcctagaca acatcaaagt |
| 41041 | ttgggctgag gtgaaaccca tccttaaaaa ttcatctcct tgatgtcttg cttaccaagg |
| 41101 | aacagtgtgt aggtcttttg gataatttga tatgaatggt tgaaagattt caaatctttg |
| 41161 | acaattaagt tgacagtgtt ttcttcgttt agaatggcat cataagtgat gctgtttcaa |
| 41221 | caaataatgc ttttgagcct gatgaggata ttgaagatct ggaggaactt gatatcccaa |
| 41281 | ccaaggaaat agagattcaa gtggattgga actctgagct tccagtcaaa gtgaacgttc |
| 41341 | ccaaagtgcc aatccatagc cttgtgcttg actgtggagc tatatctttc ctggacgttg |
| 41401 | ttggagtgag atcactgcgg gtggtaaggt tctggttttc tgaattatac atttggagct |
| 41461 | ttggcaatag taaaatgatg tgggttgtcc agtattgcaa cagggcaaat acatgggctt |
| 41521 | tgtaattttt ctaggtgaat gcttttgtaa aaaagtgtaa tattttaaag cataggctct |
| 41581 | ggagccagac tacctggggg agatactggc ttcaccactt actagcagta cgaccctggg |
| 41641 | caagttgctt aatctgtcta tatctcagtt tcttcatctg taatatggag gtaatgatgg |
| 41701 | tatctacctt cacaggttgt tacaaggatt aaataagcta atagatataa ggtgtttaga |
| 41761 | agagtgtctg gttcaggctg ggcatggtgg ctcacgcctg taatcccagc acttcgggag |
| 41821 | gctgaggcag gtggatcatg aggtcaggag ttcaagacca gcctggccaa tatggtgaaa |
| 41881 | ccccgtctct accaaaaata caaaaattag ctgggcatgg tggcgcacac ctgtagtccc |
| 41941 | agctcctagg aggctgtggc aggagaatcg cttgaacccg ggaggtggag gttgcagctg |
| 42001 | agattgtgcc actgtactcc agcctgggtg acagagtgag acttcatctc aaaaaaaaaa |
| 42061 | aaaaaaaaga atatctggtt caaagatact ccccagcaaa ttaattcaca tttattatgt |
| 42121 | tttatctttc tgagaatgta taacaagtgg tatatgaaaa gaaaaaaatg ggcaaaagtt |
| 42181 | tattaagtat tacatttcta tttgttatgt taacagcaac aggataagga ataccaggtg |
| 42241 | tatgttagga ctggaaaagc caggcattat taagaggtta gagtaggaag caggtcagac |
| 42301 | atctggaagg tcaaagcaag agttgaggag tatgcaggat agaaacatta atgatatcag |
| 42361 | aaaccagtta tgcaagctga agttcattaa ttctgtcaac aaatagatgt gaaatgccta |
| 42421 | atgtgtactg agcactctgc caggcacaag agactagtga tgaagtagac acagtttcta |
| 42481 | cctacgtggg acttagcagt ctggaggaga aggcaaacat taagtcatct tataaataac |
| 42541 | aattgtagta agtgctccaa agaagatgaa taggacatca cctgaagcat caagttcagt |
| 42601 | gaagcttcag gtaggtatca aaagggtcag gcagaaaagt aagcaggtca agacaccaac |
| 42661 | aatcagatgt aataatataa agctgactcc taactgaggt tccacttagc taaggcttct |
| 42721 | ttgctacctc tataagggga ggaaatgatt ccaggatgtg ggtactaggt aggtagggta |
| 42781 | gagaagtgat tgtgcttggc aaaagagtat gagaatatgc tgacaggttc tgttctagtt |
| 42841 | ctcttagcag attttgttat tagctcattt gaaatcactc ttgttctttg ttgtacattt |
| 42901 | cactctaagc attggctgag atccattgta gactatattg gccagagagt tcaatgaatt |
| 42961 | acttttgggg acagtcttaa catatttaat gccagaatat ttcccacaca acgaatattt |
| 43021 | actttcaaat attataatga cagttatgat agctaaagaa gactccaaac tttatacttg |
| 43081 | aacagaggtc ttgatttgtt ttcctccata attctcaaat tctcttttat tattgactat |
| 43141 | gggtcttgtg tcttgacttt ttaaaattaa atgtttattt tgacataatt gtaaattcac |
| 43201 | atgtagttgt aaggaataat atttagatcc tatatacccc ttacccagtt tctcccaatg |
| 43261 | agtaacatct tgtaaaactg tagcataaaa tcacagtagg acactgacat tcaaaataga |
| 43321 | ggccttgact tttaggtcaa tagtaacatt ttgaacccga taagcatagc cacacccata |
| 43381 | tctcttctcc aatcagcagg gcctatatta agaagcctcc tgtactctct tctttccaca |
| 43441 | aatgcttatt tagcacctcc acgctatcaa gtgcbgtgct attgagcgct ggatgttgcc |
| 43501 | atctcttgag atataatcct aattatacca ttactgtagt ttgaaaacct ccatggtttt |
| 43561 | gcaataataa cctttcctta aagtcctgat taaccatgaa agtaataatg tttctcctga |
| 43621 | gcaagtaact gaatgctact gaattatggg cagataaggt tgttaattgt tacaaactct |
| 43681 | ccttttttat ttttagattg tcaaagaatt ccaaagaatt gatgtgaatg tgtattttgc |
| 43741 | atcacttcaa ggtaaataca tatatctaca tatctacctg taagactttc ccgtaagccc |
| 43801 | tttctcctat ctgggactgt ggtcacatta tgtctgaagg cctttttttt ttttctttta |
| 43861 | aagatctcaa ttgtcattat ttgcagttct ggaatctggc actgcttcat tccataaaac |
| 43921 | agaataagtg ttccaaggaa ctaagcagga gttcagtatt atacacagaa aaggccaaag |
| 43981 | acagcaaaag caagcaacaa agagtatatt agtccattca aagttacttt cttgtaagtc |
| 44041 | agagacagtg agacagaaca ataggaaaat aatggattag ttaacatgag gtcatgtcag |
| 44101 | gatacttttt tgtatgagga ttattgcaga ggaagcttta ttattatgcc aactggaaat |
| 44161 | ttaaactgtc ctgttttagg aaatttgcta ttacctctct ctcctgattt cttcaagtat |
| 44221 | cagataacaa tttagtttag gtttgataac ttgaaacttt agcatgagtg actccatttt |
| 44281 | gatttttagt cttgtctgtt gtcgtctagt gcgggagctt agtctaaaac aatggccttt |
| 44341 | tatttttatt ttttatttta ctatgtgcct gtgccttgca tgaccccagg atgggcacag |
| 44401 | ctgcttggcc cctgagccac cgaaggctgc tctactcctt cgttctggct actaaaagct |
| 44461 | gcaaagctct gggagggcaa agctcaagaa gactaaaaca atggccttta ccatttttgt |
| 44521 | ttgatgattt tccccgtttt gatcaggctc tcacctaggt aagagtgaac aaaatgtagg |
| 44581 | agatcagtgc tactctgtta ccatcatttt gggtttctgg tctcaggagg tcatgtgtag |
| 44641 | cttatggtgc cctcctcatt atcatgtatg tctctgagtt tttgttgttt cagagagaga |
| 44701 | ccattcgatg tctgacaggt ggctttttgg aaacatttaa aactttgaga gggtataatg |
| 44761 | taccaggaag actgctatta tgactatcag ggagataata ccaagggttt atagtatgct |
| 44821 | ccttagccag gattctcatg aatcaaacca actaaaattg aatagcctga caaggagact |
| 44881 | acctgtttta accaagaagt cttctctgta gtacctgatg tatttatata tgtggaacaa |
| 44941 | gaagtgtcac ccaactgcac agatgcttcc ttgttgagtt agcaggtaat ctagcattcc |
| 45001 | atgactggtt aaattaaagc agaaagtgta agttacccaa agaagctact cattgtgaag |
| 45061 | ttttaactac agcactatcc tgccaagtga aagaggtagg cacaagtaag ggaaaattaa |
| 45121 | aagggataag catcttatga tagggagtct tgttctgaca gtcttggagc tgtctacagc |
| 45181 | atgaagttga caacttcttg ttttggtttg tagttttatt gtctctagtt gtggcatcca |
| 45241 | gcattctggc gaactctcta tgtggcccac agtttaagca tgagattcat ctcttgaaat |
| 45301 | ttacactgag ttgctcagct tcagcttaca gagcttcagg aactgagtag ttcttggtct |
| 45361 | tcattggagt gttgtagcca gatattagtg aaagctaaaa gaattaagaa tccagctcag |
| 45421 | tctacaggta gataataaaa actcataaat attgaacagg gctacagtct aagaacagtt |
| 45481 | ttactatgct tttcttttga accatatgtt tttctttcta tagtcacctc catttctatc |
| 45541 | aaagacgatc atggtagacc aatttgttta caaaataagt ttggtctcaa acttggcctg |
| 45601 | attgattatt tacacagtac agcaagaata actacatagg tgactccttt caaatttgct |
| 45661 | ttgccaagcc tggcgaggta ttgcttgcct gtgagcccag ctacttggga ggctgaggtg |
| 45721 | agaggattgc ttgagcccag gaattcaagg ctgcagtgca ctatgattgt gcctgtgaat |
| 45781 | agccactgca ctatagcctg gacaacacag tgagaccatg tctccaaaaa ataattgctt |
| 45841 | tgatagaaat tttgacaagg aatctcagat tggacttttt aaaacgtctt gatgctatga |
| 45901 | agtcaaacca aggcagacat taggctttgc ctgatgtatc taatatcttg aagttactgg |
| 45961 | gcctcccagg aaggaacgac tttttattca ctcattgtaa ggctagcagc ccttgaagcc |
| 46021 | aggaattctg tgcacatttt caaatatgat attctattca aagccttgat aatataacca |
| 46081 | atgttttcca attgtattct atttaaaaga acagattcta ttgaactttc atgtaaataa |
| 46141 | tcatattgcc ataaaaataa gaatactcac aaagagtttc caaattctgg aaggatcagg |
| 46201 | tagagaggaa aagcaaatgt ttcaattttt gtttatgaaa gtatgcttaa caaggctggg |
| 46261 | tgcggtggct cacatctgta atcccagcac tttgggaagc caaggcggac aattgctttg |
| 46321 | agctcagagt ttgagaccag cctggcaaca tggcaaaacg ccgttactac aaaaaataca |
| 46381 | aaaaattagc tgggcatgga gcctgaggct gagaatcact tgagctgggg aagtggaggt |
| 46441 | agcagtgagc ccagatggtg ccactgcact ccaacctggg tgacagagtg agaccctgtc |
| 46501 | tcaaaaaata aaaaaaaagt atgcttaacc aagtggctgt aaactgcaga tagctttaaa |
| 46561 | gaaaaatttt ctttagatct ggaaaacaaa tattaaaaga accagcaatg tttcaaataa |
| 46621 | aaaagccata aaacctgtaa ttcttctcca tcagttcatt cagtctcatg taattaattc |
| 46681 | ttgctctgtt tgatcttggc tggtagttta attccaacga atggtatgaa ttcaaagtta |
| 46741 | ttagaaacct gtatttgtca gagttctttt cattcttccc atataactct ttgaagtcac |
| 46801 | agcactttag aattataatg gcttacaaag agctttcaga aaaagtatca gaacaaaaca |
| 46861 | attaactgtg gacaacaaga cttaaagtgg ctatatttaa agatctgatg tgagttaccc |
| 46921 | aattgacaag gatatttgga tatttctgtg gcacacaaca atttaaaata accaaaatta |
| 46981 | tgactggtag catttatacc aagacctatc acatttctag gaatgttata taattttgga |
| 47041 | acatattaat aacatatcta taaaaatata gcacaaagaa agttaaacat catttcttat |
| 47101 | tttaacagtg cttcccatat aatttaacct atcagataag gccatttggt ttaacatctc |
| 47161 | tcttttacag attctttaag aaattccagg gtcctctgga acatcccaaa gttagttcaa |
| 47221 | ggtcaaaaag acttaatttt gatttttgag aagtttgtca aataccaaag gtttaaaaca |
| 47281 | tttgatcaaa atcggatcat aggtcactat gaaataaaac caaagtgaaa aaagagttca |
| 47341 | aaggcaaaaa gcacaagaag agttatattg atgaaacatg aaatctctgt tttctaggcc |
| 47401 | agttacctgg aagagaaaat cctctcacaa ttttctatta agagtaaacc aatcctctga |
| 47461 | gaaaactcta ttgttccaac acataggccc acactttagc cttccatcag tgtactttaa |
| 47521 | tattaatgct caatttttag aaaaacttat aaataattcc cttctacttt tagccaactc |
| 47581 | aatcacataa aatttttcat gatatttatc ttctacaaac cttctacaac ttgcttaaac |
| 47641 | cttcatttgg tcctatactt ccttttttaa aattggcatt gtaccttagg acaaagattt |
| 47701 | acttttcttt tctccttatc attttgacca tataaggtta tctcctatac aaaagaaaaa |
| 47761 | attactctct tttcaatttt ctttatctct tcatacttgt aaatttcttc tcacatcttt |
| 47821 | cctacctagt ggttccttcc tgccttgttt tgatttcctt cataagtcca tatttagaaa |
| 47881 | caacctttaa ataacctctg attgccaagc tagagttaag ttttaaataa ataaataggc |
| 47941 | cggtcatggc ggctcacatc tgtaatccca gcactttggg aggcaaaggt cagaggatca |
| 48001 | cttcaggcca ggagtttgag accagcctga gcaacatggt gagaccccgt ctctattatt |
| 48061 | ttttttttaa aaaacaaaaa gcataaaaac tctgagaaca ttttgaaccc agaaagatat |
| 48121 | tacttccaat ttgtgccaca gagctggtaa tgtatggaaa tgtgtatctt gacagtgggt |
| 48181 | gggggtgggg tgaatattga caatgggggg atgttgttat gaggagagca aagctggaga |
| 48241 | ttacttagat tccttagaac aggaggactc tcaagggtgt gagtctctgt gtgtttcttt |
| 48301 | atgaatgtat gtgtgtagga aagtgctaag gcttaggttg aattctttag cagcagagcc |
| 48361 | caagatgggg attcttgcac aagcgattta ttgagaatac atctgaggaa atttatgaag |
| 48421 | cagtgagggg agcaggatag ggaaggggaa gaagctgatc aaagacatgg tttctgaagt |
| 48481 | ccagcgtaga tccctgattt cacagggagc accaccacag gaagagtacc agaaggttat |
| 48541 | gcctccttga ggcaagagga cctgctttct cccccagtaa tgagtcagtc agtggctgca |
| 48601 | ggccagccat gtgtggggat gaaacctccc agttctcttt atttacttat ttatttgaga |
| 48661 | cagggtcttg ctctgtcacc caggctggag tgcagtggtg caactgtggc tcactgcagc |
| 48721 | ctcaaactcc tgggctcaag tgatcctccc atctcagcct cctgagtagc tgagactata |
| 48781 | ggtgcacaac cacacctggc taatttttgt atttttttat aaagatgggg tttcaccata |
| 48841 | ttgcccagaa gttctgggtt caaagtgatc tgccctcctc ggcctctcaa agtgctggaa |
| 48901 | ttacagaccc agtcctcttt agatgacaca gctcccaacg ggcaagggaa agtctccaga |
| 48961 | gaagggcaca gctgtgaaac attggcagcc tggcccacag cagcaggagg atggatacac |
| 49021 | tggcttggcc aaagggacgt gatcgtccac aaggttgact acgaccagtt atgggataac |
| 49081 | cattctatat actactggaa aagaaactgt catttcaaat ctgggtcaca ttttggataa |
| 49141 | gagaaaaata aataaataaa aggaggagct acaaaaactt agtataggtt gatgctttaa |
| 49201 | aatttctttt cttagcbggg catggtaggg tgtgccctgt agtcctagct aattgggagg |
| 49261 | gtgaggtggg gggatcactt gaacttggga cgcggaggtt gcagtgagca atgatgccac |
| 49321 | tgcactccag cctgggcaat agaatgagac tctgtctcaa aaacaaacaa aaatttcttt |
| 49381 | tcctaggaac taacaaaaca ttgtgtcttt cttttgaaga ttatgtgata gaaaagctgg |
| 49441 | agcaatgcgg gttctttgac gacaacatta gaaaggacac attctttttg acggtccatg |
| 49501 | atgctatact ctatctacag aaccaagtga aatctcaaga gggtcaaggt tccattttag |
| 49561 | aaacggtaaa tattcaacct ttctacagat gtatcttttc taaactatca tgatttctat |
| 49621 | aaatggcaaa cattacacaa gtctagtcta gcbgttgaat tttaagctac ctatataact |
| 49681 | tcatggagcc tcagtttttt catcagtaaa atggaagtaa aaacattaac cttgctaggt |
| 49741 | agatatgaag actaaataag atagtttata ggaaattacc tggtagtacc cagtactgaa |
| 49801 | tagttctaca atgtgtaggt ttattaataa aagtgggcat tagctataat gccttttaag |
| 49861 | aatattgaat cttggatctt ttgtgatctg ggatttgtgt acataaattc cacttaaatt |
| 49921 | ctcagcaatc tggaaaacct tgaattataa atgtctttaa acaggatcca ccaggccata |
| 49981 | agtgatcttt gaaaaagaaa tttaaaaatc aagccacaaa gaagcccagg gttaattttt |
| 50041 | ccctacaaaa tagaataggc cctgatggga ggcctcatgg cacaacagta gcgcatctgg |
| 50101 | ctccagaata ggccctgatt tcccatgggt attattttgt taatttttct gtcaaatacc |
| 50161 | cagaagaggt gagcattctg gtctggttca ttgggcttgg cctaatggtg ctcctggtgg |
| 50221 | tacaagaatc tcatttctca tagtgaattt aggaaactaa agcaaatacc tatcaagata |
| 50281 | taataacagt aatgattcca gtgacatcgt ggatatttta aattgcttcc catttttcta |
| 50341 | aaagtagaat aagtagatcc ttattgtaga ttggcagttg attgaagaaa actaacttag |
| 50401 | gaaatgatat tatattattc tttttaggca ctggtagtgg agagattgtt ctatttgatg |
| 50461 | ttccttggta aaatactagt cctaactacc aagcbgtgga tcttaaatat aaaaaaggaa |
| 50521 | aaaaaaaaaa gagtaaagct acaaatctaa agaaaaaaaa actgtttatc attgaaaatt |
| 50581 | cataaacttt ttctatccaa cttaaaattc tctaacccat aggtgataag aaagttggat |
| 50641 | catatgaagt atcctttaag agatccacat taaaagtaaa acagaaccaa ggtatcaagt |
| 50701 | tctttctcaa catgaaagca gttattttat ttgtttatac ccattcttac taagatttta |
| 50761 | ggctatgaat gtcataaaaa ccactaacca agagtgtatc tactaggtta gactcactct |
| 50821 | tgtttgcaat cagagaggta ttgtgagtgg ctgggatgga tttttttgga ccttaaacaa |
| 50881 | atagactaag cataacggaa tgcttgtatt acatacttac tggtccacta aaagtgggtt |
| 50941 | ggacattcct ggtggcctgt gagatcaaga ggcttatatt tctgtgtctt ctcagagtct |
| 51001 | agcacaaagc tttatatgtg ataagtaggt gaatgatgat aaactccttc aagagggagt |
| 51061 | ccctctgtat caaagatatt gtgactgcaa tgacacaacc ttcttcctat ccctttattt |
| 51121 | taccatcaca ggactatctc aggacacaac ttgtagttag acttatcctc tacctttccc |
| 51181 | attatgtcat accatttgca cagggactta accctctaat tcattctcat actagctaag |
| 51241 | agaattgggc tatttgtgag ttgaaaagta gctaagtagc atttcaaaat gttattttag |
| 51301 | cctggatata tatttggagt tgctttgaaa atgtcctttt ccatgcaaaa cacaaagcca |
| 51361 | aaattgtcgt tggtttcctc tgtgtagaag attaaattac atgccacctc taaacagtag |
| 51421 | agcttttctg aataaccaac ttggtccata gacattggtt tccatctcca atagaattaa |
| 51481 | tttccaccca attccatttg tggctgtttt ttgtcatcag tgacaagctc ttcactgtgc |
| 51541 | attcattgca cactcaacgc tgtgctaagt gctcttagct tagccattga gagatgcact |
| 51601 | attgactgct gaatcattta ggcagagggg gtgacttgtt aagaggcata cactcaagag |
| 51661 | gttggggaag agagctgaaa aggagtttac acaatggaga actataccag ttcacctttc |
| 51721 | aatgtgcaaa aaaatgatat acaaaaaatt ttagttggga aatataaaag aacaatacag |
| 51781 | ctgaagagga ttctgaagta tgtaaagaca gatgagaagc accaggaaag cttcaaatca |
| 51841 | ttttcagtgg agcatcaggt gggttgatgc tattctattt ctaccctgtg ttctcttttt |
| 51901 | caagatcact ctcattcagg attgtaaaga tacccttgaa ttaatagaaa cagagctgac |
| 51961 | ggaagaagaa cttgatgtcc aggatgaggt atgatcattt tcttctgaag aaaatatttg |
| 52021 | aattacattt tgaataatta gagtaataca aatagtgaat atatctgatt aagaactgtc |
| 52081 | agggaacata attcccccaa atgcagaaaa gatggcttca tagcagggaa aaagagaaaa |
| 52141 | taagaattct cccttgaaga cattaagtac ttggggatct tttgtcccag gcatttgggg |
| 52201 | atgatcagca actggcaaga gccaacacac tgctcctgat ttgataccta cacctctgca |
| 52261 | tactgctctt ctagagatgt aaggccatgg ggtaggaaaa aaatgaatag aggaaacttc |
| 52321 | taagctagga actttataag cctcaggaat agtatgtgag gaacagaaac cctatgacct |
| 52381 | tccttcatat gactgataaa ggctttaaaa acacactctc cccaccctag actgattctg |
| 52441 | tgtatttcag atgctcttgt gacatcagac tattcctagg aaacttgtct gaacttgtag |
| 52501 | catctgagta aattttgcag cttcgccact ggataagagg tgaacactga aggaagatca |
| 52561 | cagaattgta aaaagttgaa agatttctta gaatcaagta gtctaacatt ctagcccatg |
| 52621 | cagaaatcct tcctacacca tccttgatga atgttcattc agcttttatg tgaacatttc |
| 52681 | cagacattgg ccactagcgg cttctcaagg gagtttgtta catggttgga agtctctaaa |
| 52741 | tgttacttgt gcttacagaa ctcaatttgg cctttctgta gtaaccacct agtgaactga |
| 52801 | gatctgcctt ccttagcaac acagaccttc ccactgaaca gcccttcaaa tatttgagaa |
| 52861 | tagttctcat gtctccagca gactctcttc cagttgagac tcttctagtt ccttctattc |
| 52921 | cttacaaaca agcagtcagt gcttctctgg tcacgatggg gacacaaaac cctaaatgcg |
| 52981 | gccaggccag catggagatt attcaaccat gacctacctt gatctcatca agatattcct |
| 53041 | attactacag cttaaaaatg tgtttccttt cataatagca acatcagaat tttagcttct |
| 53101 | attgagcagg ggccaactaa agactggggc attttcatac aagctatggt ctagtagagt |
| 53161 | tccctagtta tatttattta attgagcttt gaccctaata cagaactaca cctatttctg |
| 53221 | ctggatttca tcttgttggt tcttgtttac tgacataatt ttgtattatt attagtatta |
| 53281 | ttattattat tattatttgt gactgagtct tactctgtca cctggactgg agtgcagtgg |
| 53341 | tacaatctca gctcactgca atccctccct cccaggttca agagattctc acgtgtgcct |
| 53401 | cagcttccca agtagttggg attacaggcg ctcgccacca cgtccagata attttttttt |
| 53461 | tttgtattct tagtagagac agggttttgc catgttgtcc aggctggtct tgaactcctg |
| 53521 | acctcaagta atccacctgc cttgccctcc cagattgctg ggattacagg catgagccac |
| 53581 | cacattggcc agttttgtgt tttgaatcta acatccagga tattgtctac cccagtaccc |
| 53641 | gttacctggt gtcataatca aatctgataa gcctgccttc tctgcctttt tctaggtctt |
| 53701 | gaaaaaatgt tgcaaagaaa acattcaacc ccctatagtg aaatcaggca gtaaggatga |
| 53761 | caggcagaga tttctgtgga ggaaatacat tgctagagtg aaaatcacag tacctgcatc |
| 53821 | ttggcccacc agcttagtga gcccaggaaa gtcattatcg acctgagaag tccttttagc |
| 53881 | tccagtagtt tatgactcta tggatatagg gtgccttagt ttgagtaaaa actcatatgt |
| 53941 | caacattaaa acttaagact gctatattat aggatattta ttggttgcca cgtaaaataa |
| 54001 | tcattcaggg agggtttcta tagatggtaa aacaatatag cacaatagtt tcaagggcat |
| 54061 | ggactttaga acaagacaga cctgggtgct agtcctcatt ctgctactta ctggctgtgc |
| 54121 | accccagggc aaattactta tcctgtcttc atattagtat cttcatgtaa agtagtaata |
| 54181 | ataccacaga gttgtgaaaa ttaaaggata aatgtaaagt aacaagagca gtgccttgta |
| 54241 | catagtaagt gctcaataaa tagtaatcat tgctcttaaa taaatccaat taaagtgtga |
| 54301 | tgaatacagg ccaggctcag tgactcaagc ctgtaatccc agcactttgg gaggctgagg |
| 54361 | cagggggatc acttgaggtc aggagtttga gaccagcctg actaacatgg taaccccccg |
| 54421 | accatctcta ctaaaaaaaa aaaaaaaaaa attagcbggg tgtggtggca ggcacctgta |
| 54481 | atcccagcta ctcgggaggc tgaggcagga gaattgcttg aacctgggag gcagaggttg |
| 54541 | cagtgagctg agatcacgcc acagcactcc agcctgggca acagtgagtg agattcagtc |
| 54601 | tccaaaaaaa aaaaaaagta taatgaatac gaaatgaagt ttttacccta tttctattgt |
| 54661 | gatgatatac acctaagatg agtagcagta agcaatcaat actataaaaa catatttata |
| 54721 | aaaaagataa tgcagactta aggagaattc agttgtatca acactttgtt ttccccttgc |
| 54781 | ttccacaggc tatgcgtaca cttgcatcct gaaagtgggt tcgggaggtc tctatgagca |
| 54841 | aggaatacaa gacaaaactt cctcaatgca ttgactattt cttcagactc aaaacactca |
| 54901 | ttcttttttc tattaagcca ttgaaagaga agcactaaga ctgcttctag gctttattta |
| 54961 | taaaataaac accttatccc taacatgggc aaaatggcta gaattattca gacgatttgg |
| 55021 | cagcgtccag ggtaagctgg tgttataata cgctgctgat ctacatcaca gatttgctaa |
| 55081 | taatgttcac gtgggccctg gcatatctct gttcagttag agtgagtgct gacccaacag |
| 55141 | cctctgtggt caagcgagtc acgaatgatt aatcataaag aaaaatcagt ttttgactga |
| 55201 | cctggatatc catgagctgc actgatcacc atgtaaggtc acatttagta aatgctgaaa |
| 55261 | taaaatgatt aatgcattta tcaataaaag cctttgaaaa tactttggat aataaattgg |
| 55321 | agttttaaaa atgcaaattt gcttagtatc taataatgaa gtgttattac atatagccgg |
| 55381 | aattgaggat ctctttgatc ctggaaatgg tttacctaaa agctacagaa ccaggccaat |
| 55441 | atattttgaa atattgatgc agacaaatga aataataaag agattttcat ggtttataaa |
| 55501 | aatctttttt gatatgataa taatcatgat cacaactgag atcaaaaaaa tatatgacag |
| 55561 | attattttgt ttaaaaatgc agttttaatt atcttagtct atagaaatga tcattgcatg |
| 55621 | gaggcatgta taggtatgat ctgtgtaaaa tctgacataa aaacagtgct attctgagtg |
| 55681 | aaaatttttt tgatgtgctt acataaccat ggtgattaaa atgagtttat attttttctc |
| 55741 | aaaaatttta gcagtgtgta aagtaagtaa tctttaactg aactctgacc acttaaaaaa |
| 55801 | aaatctaaaa attgaactac ctatagtagt ctgtgtttaa agtgaatttt taaagacaaa |
| 55861 | gcattctaaa tgaactcaat ataaaaacat tcatttggaa tgtacatact gaaaaataca |
| 55921 | ggtttttttg accaaaagtt tttatatctt ttctttttat ttattttttt cctaagtgcc |
| 55981 | aacaattttc tagatattat atacaacaca ggctttgatc ttggggactt ttcccatata |
| 56041 | tttcacactg gagtgaatga agttgtactt catttctaga gaaaagttat acccaggtcc |
| 56101 | ccaattgaga atgtcttgct tgattgaaaa cgacatcatc ccttggtata ctccagggat |
| 56161 | tggtttcagg acccctgcat ttaccaaaat ttgtgcacac tcaagtcctg cagtcacccc |
| 56221 | tgcctaaaga tagaatggct tctctgtttt tcttctgaaa tacaaccaga aacaatgtgt |
| 56281 | ctatttctga aagaatagga ttaatgatca tacaaatggg ttaatcctga attctggttg |
| 56341 | taaatctggt tacagcataa ctaggattat aatgctgcct cattttcaca gcactacttg |
| 56401 | cttatattga caacaaatca tctcgctaaa gagtgaatgt aggccaggcg cggtggctca |
| 56461 | tgcctgtaat cccagcactt tgggaggccg aggcgggtgg atcacgaggt caggagatcg |
| 56521 | agaccatcct ggctaacatg gtaaaacccc gtctctacta aaaatagaaa aaaagaaatt |
| 56581 | agcctagcgt ggtggctggc gggcgcctgt agtcccagct atttgggagg ctaaggcagg |
| 56641 | agaatggcgt gaacccggga ggcggagctt gcagtgagcc gaggtcgtgc cactgcactc |
| 56701 | cagcctgggc gacagagcaa gactccgtct caaaaaaaaa aaaaaaaaaa aaaaaagagt |
| 56761 | gaatgtaata gtcttgcaga aaatgaatga atacctttgt tcaataaagg aaatatgcac |
| 56821 | tgctcacttt tttgaaggaa atgccaaagt tacgttttac aacaaggcta gagtttgtaa |
| 56881 | attctgggtt catttgtgat gacataagtc agcaaactgc gggaatactg tctcttctat |
| 56941 | gtattttgtg aatagtaagc ataattttag ttttgtatta tcaatgaaaa tttcacttga |
| 57001 | aattaaagct gccttttgtt atatttttaa cctataggat aagattccag tattgtatat |
| 57061 | gagttttaac aaattaaaaa atcaaatcat gtacatttga aaatatttgc acacatttaa |
| 57121 | aaataaatgt aaagttgtct tttaaactac tcggatgtgt cctttctgaa caaaa |

### Otoferlin

To date, several hundred mutations in the human OTOF gene have been identified to cause profound prelingual deafness DFNB9. Such mutations are the cause of deafness in 2-8% of people that are born with an autosomal, recessively inherited, non-syndromic deafness in different populations (Rodriguez-Ballesteros et al. (2008) Hum. Mutat. 29 823-831; Choi et al. (2009) Clinical Genetics 75 237-243; Duman et al. (2011) Genet Test Mol Biomarkers 15 29-33; Varga et al. (2006) J Med Genet 43 576-581; Iwasa et al. (2013) BMC Med. Genet. 14 95). For example, two substitutions in exon 15 at positions 490 and 515 in the conserved C2C domain of otoferlin cause DFNB9 (Mirqhomizadeh et al. (2002) Neurobiol. Dis. 10(2): 157-164). Migliosi et al. found a novel mutation Q829X in OTOF in Spanish patients with prelingual non-syndromic hearing loss (Migliosi et al. (2002) J. Med. Genet. 39(7): 502-506).

Additional exemplary mutations in a otoferlin gene detected in subjects having hearing loss and methods of sequencing a nucleic acid encoding otoferlin are described in, e.g., Rodriguez-Ballesteros et al. (2003) Hum Mutat. 22: 451-456; Wang et al. (2010) BMC Med Genet. 11:79; Yildirim-Baylan et al. (2014) hit. J. Pediatr. Otorhinolaryngol 78: 950-953; Choi et al. (2009) Clin. Genet. 75(3): 237-243; and Marlin et al. (2010) Biochem Biophys Res Commun 394: 737-742.

Otoacoustic emissions from DFNB9 patients are normal, at least for the first decade of life, indicating morphological integrity of the inner ear and proper function of outer hair cells. Apart from the lack of synaptic transmission and the subsequent loss of synapses, the morphology and physiology of the inner ear remains preserved in DFNB9, at least during the first decade in life in humans.

Studies in mouse models revealed that synapses are structurally normal, and IHCs preserve normal synapse numbers in mice within the first postnatal week. Between P6 and P15, about half of the synapses get lost (Roux et al. (2006) Cell 127 277-289). Animal models allowed studying the effect of mutations in otoferlin on synaptic transmission by recording the change in plasma membrane capacitance after vesicle fusion and the activity in the auditory nerve. In otoferlin knock-out (Otof−/−) mice, almost no exocytosis could be triggered in IHCs by depolarization induced Ca2+ influx through voltage gated Ca2+ channels (Roux et al. (2006) Cell 127 277-289). In profoundly hearing impaired pachanga (Oto^{Pga/Pga}) mice with a random point mutation in the C2F domain, short (<10 ms) depolarizations of the IHCs elicited vesicle fusion of similar size as in wild type mice, however sustained stimulations uncovered a strong deficiency in replenishing vesicles to the readily releasable pool (Pangrsic et al. (2010) Nat. Neurosci. 13 869-876). The p.Ile515Thr mutation, found in human patients with only mildly elevated hearing thresholds but a severe reduction in speech understanding and a temperature-dependent deafening (Varga et al. (2006) J Med Genet 43 576-581), uncovered an intermediate phenotype when studied in a mouse model (Strenzke et al. (2016) EMBO J. 35:2519-2535). These OtofI515T/I515T mice showed a moderate elevation of hearing thresholds when assessed by ABR with a reduction in wave I amplitude, but normal auditory thresholds in behavioral tests and recordings of single auditory nerve units. Exocytosis of the RRP is again intact, but sustained exocytosis is reduced, although not as severe as in OtofPga/Pga. While in wild type mice at room temperature, during a sustained stimulus 750 vesicles can fuse per second at each active zone, this rate drops to 350 vesicles/s/active zone in OtofI515T/I515T mice and to 200 vesicles/s/active zone in OtofPga/Pga IHCs (Pangrsic et al. (2010) Nat. Neurosci. 13 869-876; Strenzke et al. (2016) EMBO J. 35 2519-2535). This correlated with lower otoferlin protein levels at the plasma membrane of IHCs, indicating that the amount of otoferlin scales with exocytosis and hearing (Strenzke et al. (2016) EMBO J. 35 2519-2535).

Methods of detecting mutations in a gene are well-known in the art. Non-limiting examples of such techniques include: real-time polymerase chain reaction (RT-PCR), PCR, sequencing, Southern blotting, and Northern blotting.

The OTOF gene encodes otoferlin, a protein that is involved in synaptic vesicle exocytosis in cochlear hair cells (see, e.g., Johnson and Chapman (2010) J. Cell Biol. 191(1):187-198; and Heidrych et al. (2008) Hum. Mol. Genet. 17:3814-3821).

The human OTOF gene is located on chromosome 2p23.3. It contains 48 exons encompassing ˜132 kilobases (kb) (NCBI Accession No. NG009937.1). The mRNA encoding the long-form of otoferlin expressed in the brain includes 48 exons (Yasunaga et al., Am. J. Hum. Genet. 67:591-600, 2000). Forward and reverse primers that can be used to amplify each of the 48 exons in the OTOF gene are described in Table 2 of Yasunaga et al., Am. J. Hum. Genet. 67:591-600, 2000. In some examples, the full-length OTOF protein is a full-length wildtype OTOF protein. The full-length wildtype OTOF protein expressed from the human OTOF gene is 1997 residues in length.

An exemplary human wildtype otoferlin protein is or includes the sequence of any one of SEQ ID NOs: 8-12. Isoform e of human otoferlin protein (SEQ ID NO: 12) is encoded by an mRNA that includes exon 48 and does not include exon 47 of the otoferlin gene (Yasunaga et al., Am. J. Hum. Genet. 67:591-600, 2000). In some embodiments, the active otoferlin protein has the sequence of SEQ ID NO: 12, but is missing the 20 amino acids including the RXR motif identified in Strenzke et al., EMBO J. 35(23):2499-2615, 2016). Non-limiting examples of nucleic acids encoding a wildtype otoferlin protein are or include any one of SEQ ID NO: 13-17. As can be appreciated in the art, at least some or all of the codons in SEQ ID NO: 13-17 can be codon-optimized to allow for optimal expression in a non-human mammal or in a human. Orthologs of human otoferlin proteins are known in the art.

Human canonical (long) isoform sequence (otoferlin protein) (also
called otoferlin isoform a) (NCBI Accession No. AAD26117.1)
(SEQ ID NO: 8)
MIKTEKSYPERRLRGVLEELSCGCCRFLSLADKDQGHSSRTRLDRERLKSCMRELENMGQQARMLRAQVKR
HTVRDKLRLCQNFLQKLRFLADEPQHSIPDIFIWMMSNNKRVAYARVPSKDLLFSIVEEETGKDCAKVKTL
FLKLPGKRGFGSAGWTVQAKVELYLWLGLSKQRKEFLCGLPCGFQEVKAAQGLGLHAFPPVSLVYTKKQAF
QLRAHMYQARSLFAADSSGLSDPFARVFFINQSQCTEVLNETLCPTWDQMLVFDNLELYGEAHELRDDPPI
IVIEIYDQDSMGKADFMGRTFAKPLVKMADEAYCPPRFPPQLEYYQIYRGNATAGDPLAAFELLQIGPAGK
ADLPPINGPVDVDRGPIMPVPMGIRPVLSKYRVEVLFWGLRDLKRVNLAQVDRPRVDIECAGKGVQSSLIH
NYKKNPNFNTLVKWFEVDLPENELLHPPLNIRVVDCRAFGRYTLVGSHAVSSLRRFIYRPPDRSAPSWNTT
GEVVVTMEPEVPIKKLETMVKLDATSEAVVKVDVAEEEKEKKKKKKGTAEEPEEEEPDESMLDWWSKYFAS
IDTMKEQLRQQEPSGIDLEEKEEVDNTEGLKGSMKGKEKARAAKEEKKKKTQSSGSGQGSEAPEKKKPKID
ELKVYPKELESEFDNFEDWLHTFNLLRGKTGDDEDGSTEEERIVGRFKGSLCVYKVPLPEDVSREAGYDST
YGMFQGIPSNDPINVLVRVYVVRATDLHPADINGKADPYIAIRLGKTDIRDKENYISKQLNPVFGKSFDIE
ASFPMESMLTVAVYDWDLVGTDDLIGETKIDLENRFYSKHRATCGIAQTYSTHGYNIWRDPMKPSQILTRL
CKDGKVDGPHFGPPGRVKVANRVFTGPSEIEDENGQRKPTDEHVALLALRHWEDIPRAGCRLVPEHVETRP
LLNPDKPGIEQGRLELWVDMFPMDMPAPGTPLDISPRKPKKYELRVIIWNTDEVVLEDDDFFTGEKSSDIF
VRGWLKGQQEDKQDTDVHYHSLTGEGNFNWRYLFPFDYLAAEEKIVISKKESMFSWDETEYKIPARLTLQI
WDADHFSADDFLGAIELDLNRFPRGAKTAKQCTMEMATGEVDVPLVSIFKQKRVKGWWPLLARNENDEFEL
TGKVEAELHLLTAEEAEKNPVGLARNEPDPLEKPNRPDTSFIWFLNPLKSARYFLWHTYRWLLLKLLLLLL
LLLLLALFLYSVPGYLVKKILGA
Human Isoform 2 (short 1) (otoferlin protein) (also called otoferlin
isoform d) (NCBI Accession No. NP_919304.1)
(SEQ ID NO: 9)
MIKTEKSYPERRLRGVLEELSCGCCRFLSLADKDQGHSSRTRLDRERLKSCMRELENMGQQARMLRAQVKR
HTVRDKLRLCQNFLQKLRFLADEPQHSIPDIFIWMMSNNKRVAYARVPSKDLLFSIVEEETGKDCAKVKTL
FLKLPGKRGFGSAGWTVQAKVELYLWLGLSKQRKEFLCGLPCGFQEVKAAQGLGLHAFPPVSLVYTKKQAF
QLRAHMYQARSLFAADSSGLSDPFARVFFINQSQCTEVLNETLCPTWDQMLVFDNLELYGEAHELRDDPPI
IVIEIYDQDSMGKADFMGRTFAKPLVKMADEAYCPPRFPPQLEYYQIYRGNATAGDLLAAFELLQIGPAGK
ADLPPINGPVDVDRGPIMPVPMGIRPVLSKYRVEVLFWGLRDLKRVNLAQVDRPRVDIECAGKGVQSSLIH
NYKKNPNFNTLVKWFEVDLPENELLHPPLNIRVVDCRAFGRYTLVGSHAVSSLRRFIYRPPDRSAPSWNTT
GEVVVTMEPEVPIKKLETMVKLDATSEAVVKVDVAEEEKEKKKKKKGTAEEPEEEEPDESMLDWWSKYFAS
IDTMKEQLRQQEPSGIDLEEKEEVDNTEGLKGSMKGKEKARAAKEEKKKKTQSSGSGQGSEAPEKKKPKID
ELKVYPKELESEFDNFEDWLHTFNLLRGKTGDDEDGSTEEERIVGRFKGSLCVYKVPLPEDVSREAGYDST
YGMFQGIPSNDPINVLVRVYVVRATDLHPADINGKADPYIAIRLGKTDIRDKENYISKQLNPVFGKSFDIE
ASFPMESMLTVAVYDWDLVGTDDLIGETKIDLENRFYSKHRATCGIAQTYSTHGYNIWRDPMKPSQILTRL
CKDGKVDGPHFGPPGRVKVANRVFTGPSEIEDENGQRKPTDEHVALLALRHWEDIPRAGCRLVPEHVETRP
LLNPDKPGIEQGRLELWVDMFPMDMPAPGTPLDISPRKPKKYELRVIIWNTDEVVLEDDDFFTGEKSSDIF
VRGWLKGQQEDKQDTDVHYHSLTGEGNFNWRYLFPFDYLAAEEKIVISKKESMFSWDETEYKIPARLTLQI
WDADHFSADDFLGAIELDLNRFPRGAKTAKQCTMEMATGEVDVPLVSIFKQKRVKGWWPLLARNENDEFEL
TGKVEAELHLLTAEEAEKNPVGLARNEPDPLEKPNRPDTAFVWFLNPLKSIKYLICTRYKWLIIKIVLALL
GLLMLGLFLYSLPGYMVKKLLGA
Human Isoform 3 (short 2) (otoferlin protein) (also called otoferlin
isoform c) (NCBI Accession No. NP_919303.1)
(SEQ ID NO: 10)
MMTDTQDGPSESSQIMRSLTPLINREEAFGEAGEAGLWPSITHTPDSQEEGLNDIQEMIKTEKSYPERRLR
GVLEELSCGCCRFLSLADKDQGHSSRTRLDRERLKSCMRELENMGQQARMLRAQVKRHTVRDKLRLCQNFL
QKLRFLADEPQHSIPDIFIWMMSNNKRVAYARVPSKDLLFSIVEEETGKDCAKVKTLFLKLPGKRGFGSAG
WTVQAKVELYLWLGLSKQRKEFLCGLPCGFQEVKAAQGLGLHAFPPVSLVYTKKQAFQLRAHMYQARSLFA
ADSSGLSDPFARVFFINQSQCTEVLNETLCPTWDQMLVETNLELYGEAHELRDDPPIIVIEIYDQDSMGKA
DFMGRTFAKPLVKMADEAYCPPRFPPQLEYYQIYRGNATAGDLLAAFELLQIGPAGKADLPPINGPVDVDR
GPIMPVPMGIRPVLSKYRVEVLFWGLRDLKRVNLAQVDRPRVDIECAGKGVQSSLIHNYKKNPNFNTLVKW
FEVDLPENELLHPPLNIRVVDCRAFGRYTLVGSHAVSSLRRFIYRPPDRSAPSWNTTVALLARCRVLCNGG
SSSHSTGEVVVTMEPEVPIKKLETMVKLDATSEAVVKVDVAEEEKEKKKKKKGTAEEPEEEEPDESMLDWW
SKYFASIDTMKEQLRQQEPSGIDLEEKEEVDNTEGLKGSMKGKEKARAAKEEKKKKTQSSGSGQGSEAPEK
KKPKIDELKVYPKELESEFDNFEDWLHTFNLLRGKTGDDEDGSTEEERIVGRFKGSLCVYKVPLPEDVSRE
AGYDSTYGMFQGIPSNDPINVLVRVYVVRATDLHPADINGKADPYIAIRLGKTDIRDKENYISKQLNPVFG
KSFDIEASFPMESMLTVAVYDWDLVGTDDLIGETKIDLENRFYSKHRATCGIAQTYSTHGYNIWRDPMKPS
QILTRLCKDGKVDGPHFGPPGRVKVANRVFTGPSEIEDENGQRKPTDEVALLALRHWEDIPRAGCRLVPE
HVETRPLLNPDKPGIEQGRLELWVDMFPMDMPAPGTPLDISPRKPKKYELRVIIWNTDEVVLEDDDFFTGE
KSSDIFVRGWLKGQQEDKQDTDVHYHSLTGEGNFNWRYLFPFDYLAAEEKIVISKKESMFSWDETEYKIPA
RLTLQIWDADHFSADDFLGAIELDLNRFPRGAKTAKQCTMEMATGEVDVPLVSIFKQKRVKGWWPLLARNE
NDEFELTGKVEAELHLLTAEEAEKNPVGLARNEPDPLEKPNRPDTSFIWFLNPLKSARYFLWHTYRWLLLK
LLLLLLLLLLLALFLYSVPGYLVKKILGA
Human Isoform 4 (short 3) (otoferlin protein) (also called otoferlin
isoform b) (NCBI Accession No. NP_004793.2)
(SEQ ID NO: 11)
MIKTEKSYPERRLRGVLEELSCGCCRFLSLADKDQGHSSRTRLDRERLKSCMRELENMGQQARMLRAQVKR
HTVRDKLRLCQNFLQKLRFLADEPQHSIPDIFIWMMSNNKRVAYARVPSKDLLFSIVEEETGKDCAKVKTL
FLKLPGKRGFGSAGWTVQAKVELYLWLGLSKQRKEFLCGLPCGFQEVKAAQGLGLHAFPPVSLVYTKKQAF
QLRAHMYQARSLFAADSSGLSDPFARVFFINQSQCTEVLNETLCPTWDQMLVFDNLELYGEAHELRDDPPI
IVIEIYDQDSMGKADFMGRTFAKPLVKMADEAYCPPRFPPQLEYYQIYRGNATAGDLLAAFELLQIGPAGK
ADLPPINGPVDVDRGPIMPVPMGIRPVLSKYRVEVLFWGLRDLKRVNLAQVDRPRVDIECAGKGVQSSLIH
NYKKNPNFNTLVKWFEVDLPENELLHPPLNIRVVDCRAFGRYTLVGSHAVSSLRRFIYRPPDRSAPSWNTT
GEVVVTMEPEVPIKKLETMVKLDATSEAVVKVDVAEEEKEKKKKKKGTAEEPEEEEPDESMLDWWSKYFAS
IDTMKEQLRQQEPSGIDLEEKEEVDNTEGLKGSMKGKEKARAAKEEKKKKTQSSGSGQGSEAPEKKKPKID
ELKVYPKELESEFDNFEDWLHTFNLLRGKTGDDEDGSTEEERIVGRFKGSLCVYKVPLPEDVSREAGYDST
YGMFQGIPSNDPINVLVRVYVVRATDLHPADINGKADPYIAIRLGKTDIRDKENYISKQLNPVFGKSFDIE
ASFPMESMLTVAVYDWDLVGTDDLIGETKIDLENRFYSKHRATCGIAQTYSTHGYNIWRDPMKPSQILTRL
CKDGKVDGPHFGPPGRVKVANRVFTGPSEIEDENGQRKPTDEHVALLALRHWEDIPRAGCRLVPEHVETRP
LLNPDKPGIEQGRLELWVDMFPMDMPAPGTPLDISPRKPKKYELRVIIWNTDEVVLEDDDFFTGEKSSDIF
VRGWLKGQQEDKQDTDVHYHSLTGEGNFNWRYLFPFDYLAAEEKIVISKKESMFSWDETEYKIPARLTLQI
WDADHFSADDFLGAIELDLNRFPRGAKTAKQCTMEMATGEVDVPLVSIFKQKRVKGWWPLLARNENDEFEL
TGKVEAELHLLTAEEAEKNPVGLARNEPDPLEKPNRPDTSFIWFLNPLKSARYFLWHTYRWLLLKLLLLLL
LLLLLALFLYSVPGYLVKKILGA
Human Isoform 5 (short 4) (otoferlin protein) (also called otoferlin
isoform e) (NCBI Accession No. NP_001274418.1)
(SEQ ID NO: 12)
MALLIHLKTVSELRGRGDRIAKVTFRGQSFYSRVLENCEDVADFDETFRWPVASSIDRNEMLEIQVFNYSK
VFSNKLIGTFRMVLQKVVEESHVEVTDTLIDDNNAIIKTSLCVEVRYQATDGTVGSWDDGDFLGDESLQEE
EKDSQETDGLLPGSRPSSRPPGEKSFRRAGRSVFSAMKLGKNRSHKEEPQRPDEPAVLEMEDLDHLAIRLG
DGLDPDSVSLASVTALTTNVSNKRSKPDIKMEPSAGRPMDYQVSITVIEARQLVGLNMDPVVCVEVGDDKK
YTSMKESTNCPYYNEYFVFDFHVSPDVMFDKIIKISVIHSKNLLRSGTLVGSFKMDVGTVYSQPEHQFHHK
WAILSDPDDISSGLKGYVKCDVAVVGKGDNIKTPHKANETDEDDIEGNLLLPEGVPPERQWARFYVKIYRA
EGLPRMNTSLMANVKKAFIGENKDLVDPYVQVFFAGQKGKTSVQKSSYEPLWNEQVVFTDLFPPLCKRMKV
QIRDSDKVNDVAIGTHFIDLRKISNDGDKGFLPTLGPAWVNMYGSTRNYTLLDEHQDLNEGLGEGVSFRAR
LLLGLAVEIVDTSNPELTSSTEVQVEQATPISESCAGKMEEFFLFGAFLEASMIDRRNGDKPITFEVTIGN
YGNEVDGLSRPQRPRPRKEPGDEEEVDLIQNASDDEAGDAGDLASVSSTPPMRPQVTDRNYFHLPYLERKP
CIYIKSWWPDQRRRLYNANIMDHIADKLEEGLNDIQEMIKTEKSYPERRLRGVLEELSCGCCRFLSLADKD
QGHSSRTRLDRERLKSCMRELENMGQQARMLRAQVKRHTVRDKLRLCQNFLQKLRFLADEPQHSIPDIFIW
MMSNNKRVAYARVPSKDLLFSIVEEETGKDCAKVKTLFLKLPGKRGFGSAGWTVQAKVELYLWLGLSKQRK
EFLCGLPCGFQEVKAAQGLGLHAFPPVSLVYTKKQAFQLRAHMYQARSLFAADSSGLSDPFARVFFINQSQ
CTEVLNETLCPTWDQMLVFDNLELYGEAHELRDDPPIIVIEIYDQDSMGKADFMGRTFAKPLVKMADEAYC
PPRFPPQLEYYQIYRGNATAGDLLAAFELLQIGPAGKADLPPINGPVDVDRGPIMPVPMGIRPVLSKYRVE
VLFWGLRDLKRVNLAQVDRPRVDIECAGKGVQSSLIHNYKKNPNFNTLVKWFEVDLPENELLHPPLNIRVV
DCRAFGRYTLVGSHAVSSLRRFIYRPPDRSAPSWNTTVRLLRRCRVLCNGGSSSHSTGEVVVTMEPEVPIK
KLETMVKLDATSEAVVKVDVAEEEKEKKKKKKGTAEEPEEEEPDESMLDWWSKYFASIDTMKEQLRQQEPS
GIDLEEKEEVDNTEGLKGSMKGKEKARAAKEEKKKKTQSSGSGQGSEAPEKKKPKIDELKVYPKELESEFD
NFEDWLHTFNLLRGKTGDDEDGSTEEERIVGRFKGSLCVYKVPLPEDVSREAGYDSTYGMFQGIPSNDPIN
VLVRVYVVRATDLHPADINGKADPYIAIRLGKTDIRDKENYISKQLNPVFGKSFDIEASFPMESMLTVAVY
DWDLVGTDDLIGETKIDLENRFYSKHRATCGIAQTYSTHGYNIWRDPMKPSQILTRLCKDGKVDGPHFGPP
GRVKVANRVFTGPSEIEDENGQRKPTDEHVALLALRHWEDIPRAGCRLVPEHVETRPLLNPDKPGIEQGRL
ELWVDMFPMDMPAPGTPLDISPRKPKKYELRVIIWNTDEVVLEDDDFFTGEKSSDIFVRGWLKGQQEDKQD
TDVHYHSLTGEGNFNWRYLFPFDYLAAEEKIVISKKESMFSWDETEYKIPARLTLQIWDADHFSADDFLGA
IELDLNRFPRGAKTAKQCTMEMATGEVDVPLVSIFKQKRVKGWWPLLARNENDEFELTGKVEAELHLLTAE
EAEKNPVGLARNEPDPLEKPNRPDTAFVWFLNPLKSIKYLICTRYKWLIIKIVLALLGLLMLGLFLYSLPG
YMVKKLLGA
Complete cds (otoferlin cDNA) (encodes the protein of SEQ ID NO: 8)
(SEQ ID NO: 13)
atga tcaaaacgga gaagtcctac cctgagcgtc gcctgcgggg cgtcctggag gagctgagct
gtggctgctg ccgcttcctc tccctcgctg acaaggacca gggccactca tcccgcacca
ggcttgaccg ggagcgcctc aagtcctgca tgagggagct ggaaaacatg gggcagcagg
ccaggatgct gcgggcccag gtgaagcggc acacggtgcg ggacaagctg aggctgtgcc
agaacttcct gcagaagctg cgcttcctgg cggacgagcc ccagcacagc attcccgaca
tcttcatctg gatgatgagc aacaacaagc gtgtcgccta tgcccgtgtg ccctccaagg
acctgctctt ctccatcgtg gaggaggaga ctggcaagga ctgcgccaag gtcaagacgc
tcttccttaa gctgccaggg aagcggggct tcggctcggc aggctggaca gtgcaggcca
aggtggagct gtacctgtgg ctgggcctca gcaaacagcg caaggagttc ctgtgcggcc
tgccctgtgg cttccaggag gtcaaggcag cccagggcct gggcctgcat gccttcccac
ccgtcagcct ggtctacacc aagaagcagg cgttccagct ccgagcgcac atgtaccagg
cccgcagcct ctttgccgcc gacagcagcg gactctcaga cccctttgcc cgcgtcttct
tcatcaatca gagtcagtgc acagaggtgc tgaatgagac cctgtgtccc acctgggacc
agatgctggt gttcgacaac ctggagctct atggtgaagc tcatgagctg agggacgatc
cgcccatcat tgtcattgaa atctatgacc aggattccat gggcaaagct gacttcatgg
gccggacctt cgccaaaccc ctggtgaaga tggcagacga ggcgtactgc ccaccccgct
tcccacctca gctcgagtac taccagatct accgtggcaa cgccacagct ggagacccgc
tggcggcctt cgagctgctg cagattggac cagcagggaa ggctgacctg ccccccatca
atggcccggt ggacgtggac cgaggtccca tcatgcccgt gcccatgggc atccggcccg
tgctcagcaa gtaccgagtg gaggtgctgt tctggggcct acgggaccta aagcgggtga
acctggccca ggtggaccgg ccacgggtgg acatcgagtg tgcagggaag ggggtgcagt
cgtccctgat ccacaattat aagaagaacc ccaacttcaa caccctcgtc aagtggtttg
aagtggacct cccagagaac gagctgctgc acccgccctt gaacatccgt gtggtggact
gccgggcctt cggtcgctac acactggtgg gctcccatgc cgtcagctcc ctgcgacgct
tcatctaccg gcccccagac cgctcggccc ccagctggaa caccacgggg gaggttgtgg
tgactatgga gccagaggta cccatcaaga aactggagac catggtgaag ctggacgcga
cttctgaagc tgttgtcaag gtggatgtgg ctgaggagga gaaggagaag aagaagaaga
agaagggcac tgcggaggag ccagaggagg aggagccaga cgagagcatg ctggactggt
ggtccaagta ctttgcctcc attgacacca tgaaggagca acttcgacaa caagagccct
ctggaattga cttggaggag aaggaggaag tggacaatac cgagggcctg aaggggtcaa
tgaagggcaa ggagaaggca agggctgcca aagaggagaa gaagaagaaa actcagagct
ctggctctgg ccaggggtcc gaggcccccg agaagaagaa acccaagatt gatgagctta
aggtataccc caaagagctg gagtccgagt ttgataactt tgaggactgg ctgcacactt
tcaacttgct tcggggcaag accggggatg atgaggatgg ctccaccgag gaggagcgca
ttgtgggacg cttcaagggc tccctctgcg tgtacaaagt gccactccca gaggacgtgt
cccgggaagc cggctacgac tccacctacg gcatgttcca gggcatcccg agcaatgacc
ccatcaatgt gctggtccga gtctatgtgg tccgggccac ggacctgcac cctgctgaca
tcaacggcaa agctgacccc tacatcgcca tccggctagg caagactgac atccgcgaca
aggagaacta catctccaag cagctcaacc ctgtctttgg gaagtccttt gacatcgagg
cctccttccc catggaatcc atgctgacgg tggctgtgta tgactgggac ctggtgggca
ctgatgacct cattggggaa accaagatcg acctggagaa ccgcttctac agcaagcacc
gcgccacctg cggcatcgcc cagacctact ccacacatgg ctacaatatc tggcgggacc
ccatgaagcc cagccagatc ctgacccgcc tctgcaaaga cggcaaagtg gacggccccc
actttgggcc ccctgggaga gtgaaggtgg ccaaccgcgt cttcactggg ccctctgaga
ttgaggacga gaacggtcag aggaagccca cagacgagca tgtggcgctg ttggccctga
ggcactggga ggacatcccc cgcgcaggct gccgcctggt gccagagcat gtggagacga
ggccgctgct caaccccgac aagccgggca tcgagcaggg ccgcctggag ctgtgggtgg
acatgttccc catggacatg ccagcccctg ggacgcctct ggacatctca cctcggaagc
ccaagaagta cgagctgcgg gtcatcatct ggaacacaga tgaggtggtc ttggaggacg
acgacttctt cacaggggag aagtccagtg acatcttcgt gagggggtgg ctgaagggcc
agcaggagga caagcaggac acagacgtcc actaccactc cctcactggc gagggcaact
tcaactggcg ctacctgttc cccttcgact acctggcggc ggaggagaag atcgtcatct
ccaagaagga gtccatgttc tcctgggacg agaccgagta caagatcccc gcgcggctca
ccctgcagat ctgggatgcg gaccacttct ccgctgacga cttcctgggg gccatcgagc
tggacctgaa ccggttcccg cggggcgcaa agacagccaa gcagtgcacc atggagatgg
ccaccgggga ggtggacgtg cccctcgtgt ccatcttcaa gcaaaagcgc gtcaaaggct
ggtggcccct cctggcccgc aatgagaacg atgagtttga gctcacgggc aaggtggagg
ctgagctgca tttactgaca gcagaggagg cagagaagaa cccagtgggc ctggcccgca
atgaacctga ccccctagag aaacccaacc ggcccgacac gagcttcatc tggttcctga
accctctcaa gtcggctcgc tacttcttgt ggcacacgta tcgctggctg ctcctcaaac
tgttgctgct cctgctgctg ctcctcctcc tcgccctgtt cctctactct gtgcctggct
acctggtcaa gaaaatcctc ggggcctga
Human Otoferlin Transcript Variant 1 (encodes the protein of SEQ ID NO: 8)
(SEQ ID NO: 14)
atggccttgctca tccacctcaa gacagtctcg gagctgcggg gcaggggcga ccggatcgcc
aaagtgactt tccgagggca atccttctac tctcgggtcc tggagaactg tgaggatgtg
gctgactttg atgagacatt tcggtggccg gtggccagca gcatcgacag aaatgagatg
ctggagattc aggttttcaa ctacagcaaa gtcttcagca acaagctcat cgggaccttc
cgcatggtgc tgcagaaggt ggtagaggag agccatgtgg aggtgactga cacgctgatt
gatgacaaca atgctatcat caagaccagc ctgtgcgtgg aggtccggta tcaggccact
gacggcacag tgggctcctg ggacgatggg gacttcctgg gagatgagtc tcttcaagag
gaagagaagg acagccaaga gacggatgga ctgctcccag gctcccggcc cagctcccgg
cccccaggag agaagagctt ccggagagcc gggaggagcg tgttctccgc catgaagctc
ggcaaaaacc ggtctcacaa ggaggagccc caaagaccag atgaaccggc ggtgctggag
atggaagacc ttgaccatct ggccattcgg ctaggagatg gactggatcc cgactcggtg
tctctagcct cagtcacagc tctcaccact aatgtctcca acaagcgatc taagccagac
attaagatgg agccaagtgc tgggcggccc atggattacc aggtcagcat cacggtgatc
gaggcccggc agctggtggg cttgaacatg gaccctgtgg tgtgcgtgga ggtgggtgac
gacaagaagt acacatccat gaaggagtcc actaactgcc cctattacaa cgagtacttc
gtcttcgact tccatgtctc tccggatgtc atgtttgaca agatcatcaa gatttcggtg
attcactcca agaacctgct gcgcagtggc accctggtgg gctccttcaa aatggacgtg
ggaaccgtgt actcgcagcc agagcaccag ttccatcaca agtgggccat cctgtctgac
cccgatgaca tctcctcggg gctgaagggc tacgtgaagt gtgacgttgc cgtggtgggc
aaaggggaca acatcaagac gccccacaag gccaatgaga ccgacgaaga tgacattgag
gggaacttgc tgctccccga gggggtgccc cccgaacgcc agtgggcccg gttctatgtg
aaaatttacc gagcagaggg gctgccccgt atgaacacaa gcctcatggc caatgtaaag
aaggctttca tcggtgaaaa caaggacctc gtggacccct acgtgcaagt cttctttgct
ggccagaagg gcaagacttc agtgcagaag agcagctatg agcccctgtg gaatgagcag
gtcgtcttta cagacctctt ccccccactc tgcaaacgca tgaaggtgca gatccgagac
tcggacaagg tcaacgacgt ggccatcggc acccacttca ttgacctgcg caagatttct
aatgacggag acaaaggctt cctgcccaca ctgggcccag cctgggtgaa catgtacggc
tccacacgta actacacgct gctggatgag catcaggacc tgaacgaggg cctgggggag
ggtgtgtcct tccgggcccg gctcctgctg ggcctggctg tggagatcgt agacacctcc
aaccctgagc tcaccagctc cacagaggtg caggtggagc aggccacgcc catctcggag
agctgtgcag gtaaaatgga agaattcttt ctctttggag ccttcctgga ggcctcaatg
atcgaccgga gaaacggaga caagcccatc acctttgagg tcaccatagg caactatggg
aacgaagttg atggcctgtc ccggccccag cggcctcggc cccggaagga gccgggggat
gaggaagaag tagacctgat tcagaacgca agtgatgacg aggccggtga tgccggggac
ctggcctcag tctcctccac tccaccaatg cggccccagg tcaccgacag gaactacttc
catctgccct acctggagcg aaagccctgc atctacatca agagctggtg gccggaccag
cgccgccgcc tctacaatgc caacatcatg gaccacattg ccgacaagct ggaagaaggc
ctgaacgaca tacaggagat gatcaaaacg gagaagtcct accctgagcg tcgcctgcgg
ggcgtcctgg aggagctgag ctgtggctgc tgccgcttcc tctccctcgc tgacaaggac
cagggccact catcccgcac caggcttgac cgggagcgcc tcaagtcctg catgagggag
ctggaaaaca tggggcagca ggccaggatg ctgcgggccc aggtgaagcg gcacacggtg
cgggacaagc tgaggctgtg ccagaacttc ctgcagaagc tgcgcttcct ggcggacgag
ccccagcaca gcattcccga catcttcatc tggatgatga gcaacaacaa gcgtgtcgcc
tatgcccgtg tgccctccaa ggacctgctc ttctccatcg tggaggagga gactggcaag
gactgcgcca aggtcaagac gctcttcctt aagctgccag ggaagcgggg cttcggctcg
gcaggctgga cagtgcaggc caaggtggag ctgtacctgt ggctgggcct cagcaaacag
cgcaaggagt tcctgtgcgg cctgccctgt ggcttccagg aggtcaaggc agcccagggc
ctgggcctgc atgccttccc acccgtcagc ctggtctaca ccaagaagca ggcgttccag
ctccgagcgc acatgtacca ggcccgcagc ctctttgccg ccgacagcag cggactctca
gacccctttg cccgcgtctt cttcatcaat cagagtcagt gcacagaggt gctgaatgag
accctgtgtc ccacctggga ccagatgctg gtgttcgaca acctggagct ctatggtgaa
gctcatgagc tgagggacga tccgcccatc attgtcattg aaatctatga ccaggattcc
atgggcaaag ctgacttcat gggccggacc ttcgccaaac ccctggtgaa gatggcagac
gaggcgtact gcccaccccg cttcccacct cagctcgagt actaccagat ctaccgtggc
aacgccacag ctggagacct gctggcggcc ttcgagctgc tgcagattgg accagcaggg
aaggctgacc tgccccccat caatggcccg gtggacgtgg accgaggtcc catcatgccc
gtgcccatgg gcatccggcc cgtgctcagc aagtaccgag tggaggtgct gttctggggc
ctacgggacc taaagcgggt gaacctggcc caggtggacc ggccacgggt ggacatcgag
tgtgcaggga agggggtgca gtcgtccctg atccacaatt ataagaagaa ccccaacttc
aacaccctcg tcaagtggtt tgaagtggac ctcccagaga acgagctgct gcacccgccc
ttgaacatcc gtgtggtgga ctgccgggcc ttcggtcgct acacactggt gggctcccat
gccgtcagct ccctgcgacg cttcatctac cggcccccag accgctcggc ccccagctgg
aacaccacgg tcaggcttct ccggcgctgc cgtgtgctgt gcaatggggg ctcctcctct
cactccacag gggaggttgt ggtgactatg gagccagagg tacccatcaa gaaactggag
accatggtga agctggacgc gacttctgaa gctgttgtca aggtggatgt ggctgaggag
gagaaggaga agaagaagaa gaagaagggc actgcggagg agccagagga ggaggagcca
gacgagagca tgctggactg gtggtccaag tactttgcct ccattgacac catgaaggag
caacttcgac aacaagagcc ctctggaatt gacttggagg agaaggagga agtggacaat
accgagggcc tgaaggggtc aatgaagggc aaggagaagg caagggctgc caaagaggag
aagaagaaga aaactcagag ctctggctct ggccaggggt ccgaggcccc cgagaagaag
aaacccaaga ttgatgagct taaggtatac cccaaagagc tggagtccga gtttgataac
tttgaggact ggctgcacac tttcaacttg cttcggggca agaccgggga tgatgaggat
ggctccaccg aggaggagcg cattgtggga cgcttcaagg gctccctctg cgtgtacaaa
gtgccactcc cagaggacgt gtcccgggaa gccggctacg actccaccta cggcatgttc
cagggcatcc cgagcaatga ccccatcaat gtgctggtcc gagtctatgt ggtccgggcc
acggacctgc accctgctga catcaacggc aaagctgacc cctacatcgc catccggcta
ggcaagactg acatccgcga caaggagaac tacatctcca agcagctcaa ccctgtcttt
gggaagtcct ttgacatcga ggcctccttc cccatggaat ccatgctgac ggtggctgtg
tatgactggg acctggtggg cactgatgac ctcattgggg aaaccaagat cgacctggag
aaccgcttct acagcaagca ccgcgccacc tgcggcatcg cccagaccta ctccacacat
ggctacaata tctggcggga ccccatgaag cccagccaga tcctgacccg cctctgcaaa
gacggcaaag tggacggccc ccactttggg ccccctggga gagtgaaggt ggccaaccgc
gtcttcactg ggccctctga gattgaggac gagaacggtc agaggaagcc cacagacgag
catgtggcgc tgttggccct gaggcactgg gaggacatcc cccgcgcagg ctgccgcctg
gtgccagagc atgtggagac gaggccgctg ctcaaccccg acaagccggg catcgagcag
ggccgcctgg agctgtgggt ggacatgttc cccatggaca tgccagcccc tgggacgcct
ctggacatct cacctcggaa gcccaagaag tacgagctgc gggtcatcat ctggaacaca
gatgaggtgg tcttggagga cgacgacttc ttcacagggg agaagtccag tgacatcttc
gtgagggggt ggctgaaggg ccagcaggag gacaagcagg acacagacgt ccactaccac
tccctcactg gcgagggcaa cttcaactgg cgctacctgt tccccttcga ctacctggcg
gcggaggaga agatcgtcat ctccaagaag gagtccatgt tctcctggga cgagaccgag
tacaagatcc ccgcgcggct caccctgcag atctgggatg cggaccactt ctccgctgac
gacttcctgg gggccatcga gctggacctg aaccggttcc cgcggggcgc aaagacagcc
aagcagtgca ccatggagat ggccaccggg gaggtggacg tgcccctcgt gtccatcttc
aagcaaaagc gcgtcaaagg ctggtggccc ctcctggccc gcaatgagaa cgatgagttt
gagctcacgg gcaaggtgga ggctgagctg catttactga cagcagagga ggcagagaag
aacccagtgg gcctggcccg caatgaacct gaccccctag agaaacccaa ccggcccgac
acgagcttca tctggttcct gaaccctctc aagtcggctc gctacttctt gtggcacacg
tatcgctggc tgctcctcaa actgttgctg ctcctgctgc tgctcctcct cctcgccctg
ttcctctact ctgtgcctgg ctacctggtc aagaaaatcc tcggggcctg a
Human Otoferlin Transcript Variant 2 (encodes the protein of SEQ ID NO: 11)
(SEQ ID NO: 15)
atgatcaaaacgga gaagtcctac cctgagcgtc gcctgcgggg cgtcctggag gagctgagct
gtggctgctg ccgcttcctc tccctcgctg acaaggacca gggccactca tcccgcacca
ggcttgaccg ggagcgcctc aagtcctgca tgagggagct ggaaaacatg gggcagcagg
ccaggatgct gcgggcccag gtgaagcggc acacggtgcg ggacaagctg aggctgtgcc
agaacttcct gcagaagctg cgcttcctgg cggacgagcc ccagcacagc attcccgaca
tcttcatctg gatgatgagc aacaacaagc gtgtcgccta tgcccgtgtg ccctccaagg
acctgctctt ctccatcgtg gaggaggaga ctggcaagga ctgcgccaag gtcaagacgc
tcttccttaa gctgccaggg aagcggggct tcggctcggc aggctggaca gtgcaggcca
aggtggagct gtacctgtgg ctgggcctca gcaaacagcg caaggagttc ctgtgcggcc
tgccctgtgg cttccaggag gtcaaggcag cccagggcct gggcctgcat gccttcccac
ccgtcagcct ggtctacacc aagaagcagg cgttccagct ccgagcgcac atgtaccagg
cccgcagcct ctttgccgcc gacagcagcg gactctcaga cccctttgcc cgcgtcttct
tcatcaatca gagtcagtgc acagaggtgc tgaatgagac cctgtgtccc acctgggacc
agatgctggt gttcgacaac ctggagctct atggtgaagc tcatgagctg agggacgatc
cgcccatcat tgtcattgaa atctatgacc aggattccat gggcaaagct gacttcatgg
gccggacctt cgccaaaccc ctggtgaaga tggcagacga ggcgtactgc ccaccccgct
tcccacctca gctcgagtac taccagatct accgtggcaa cgccacagct ggagacctgc
tggcggcctt cgagctgctg cagattggac cagcagggaa ggctgacctg ccccccatca
atggcccggt ggacgtggac cgaggtccca tcatgcccgt gcccatgggc atccggcccg
tgctcagcaa gtaccgagtg gaggtgctgt tctggggcct acgggaccta aagcgggtga
acctggccca ggtggaccgg ccacgggtgg acatcgagtg tgcagggaag ggggtgcagt
cgtccctgat ccacaattat aagaagaacc ccaacttcaa caccctcgtc aagtggtttg
aagtggacct cccagagaac gagctgctgc acccgccctt gaacatccgt gtggtggact
gccgggcctt cggtcgctac acactggtgg gctcccatgc cgtcagctcc ctgcgacgct
tcatctaccg gcccccagac cgctcggccc ccagctggaa caccacgggg gaggttgtgg
tgactatgga gccagaggta cccatcaaga aactggagac catggtgaag ctggacgcga
cttctgaagc tgttgtcaag gtggatgtgg ctgaggagga gaaggagaag aagaagaaga
agaagggcac tgcggaggag ccagaggagg aggagccaga cgagagcatg ctggactggt
ggtccaagta ctttgcctcc attgacacca tgaaggagca acttcgacaa caagagccct
ctggaattga cttggaggag aaggaggaag tggacaatac cgagggcctg aaggggtcaa
tgaagggcaa ggagaaggca agggctgcca aagaggagaa gaagaagaaa actcagagct
ctggctctgg ccaggggtcc gaggcccccg agaagaagaa acccaagatt gatgagctta
aggtataccc caaagagctg gagtccgagt ttgataactt tgaggactgg ctgcacactt
tcaacttgct tcggggcaag accggggatg atgaggatgg ctccaccgag gaggagcgca
ttgtgggacg cttcaagggc tccctctgcg tgtacaaagt gccactccca gaggacgtgt
cccgggaagc cggctacgac tccacctacg gcatgttcca gggcatcccg agcaatgacc
ccatcaatgt gctggtccga gtctatgtgg tccgggccac ggacctgcac cctgctgaca
tcaacggcaa agctgacccc tacatcgcca tccggctagg caagactgac atccgcgaca
aggagaacta catctccaag cagctcaacc ctgtctttgg gaagtccttt gacatcgagg
cctccttccc catggaatcc atgctgacgg tggctgtgta tgactgggac ctggtgggca
ctgatgacct cattggggaa accaagatcg acctggagaa ccgcttctac agcaagcacc
gcgccacctg cggcatcgcc cagacctact ccacacatgg ctacaatatc tggcgggacc
ccatgaagcc cagccagatc ctgacccgcc tctgcaaaga cggcaaagtg gacggccccc
actttgggcc ccctgggaga gtgaaggtgg ccaaccgcgt cttcactggg ccctctgaga
ttgaggacga gaacggtcag aggaagccca cagacgagca tgtggcgctg ttggccctga
ggcactggga ggacatcccc cgcgcaggct gccgcctggt gccagagcat gtggagacga
ggccgctgct caaccccgac aagccgggca tcgagcaggg ccgcctggag ctgtgggtgg
acatgttccc catggacatg ccagcccctg ggacgcctct ggacatctca cctcggaagc
ccaagaagta cgagctgcgg gtcatcatct ggaacacaga tgaggtggtc ttggaggacg
acgacttctt cacaggggag aagtccagtg acatcttcgt gagggggtgg ctgaagggcc
agcaggagga caagcaggac acagacgtcc actaccactc cctcactggc gagggcaact
tcaactggcg ctacctgttc cccttcgact acctggcggc ggaggagaag atcgtcatct
ccaagaagga gtccatgttc tcctgggacg agaccgagta caagatcccc gcgcggctca
ccctgcagat ctgggatgcg gaccacttct ccgctgacga cttcctgggg gccatcgagc
tggacctgaa ccggttcccg cggggcgcaa agacagccaa gcagtgcacc atggagatgg
ccaccgggga ggtggacgtg cccctcgtgt ccatcttcaa gcaaaagcgc gtcaaaggct
ggtggcccct cctggcccgc aatgagaacg atgagtttga gctcacgggc aaggtggagg
ctgagctgca tttactgaca gcagaggagg cagagaagaa cccagtgggc ctggcccgca
atgaacctga ccccctagag aaacccaacc ggcccgacac gagcttcatc tggttcctga
accctctcaa gtcggctcgc tacttcttgt ggcacacgta tcgctggctg ctcctcaaac
tgttgctgct cctgctgctg ctcctcctcc tcgccctgtt cctctactct gtgcctggct
acctggtcaa gaaaatcctc ggggcctga
Human Otoferlin Transcript Variant 3 (encodes the protein of SEQ ID NO: 8)
(SEQ ID NO: 16)
atgatgaccga tactcaggat ggccctagcg agagctccca gatcatgagg tccctcactc
ccctgatcaa cagggaggag gcatttgggg aggctgggga ggcggggctg tggcccagca
tcacccacac tcctgattca caggaagaag gcctgaacga catacaggag atgatcaaaa
cggagaagtc ctaccctgag cgtcgcctgc ggggcgtcct ggaggagctg agctgtggct
gctgccgctt cctctccctc gctgacaagg accagggcca ctcatcccgc accaggcttg
accgggagcg cctcaagtcc tgcatgaggg agctggaaaa catggggcag caggccagga
tgctgcgggc ccaggtgaag cggcacacgg tgcgggacaa gctgaggctg tgccagaact
tcctgcagaa gctgcgcttc ctggcggacg agccccagca cagcattccc gacatcttca
tctggatgat gagcaacaac aagcgtgtcg cctatgcccg tgtgccctcc aaggacctgc
tcttctccat cgtggaggag gagactggca aggactgcgc caaggtcaag acgctcttcc
ttaagctgcc agggaagcgg ggcttcggct cggcaggctg gacagtgcag gccaaggtgg
agctgtacct gtggctgggc ctcagcaaac agcgcaagga gttcctgtgc ggcctgccct
gtggcttcca ggaggtcaag gcagcccagg gcctgggcct gcatgccttc ccacccgtca
gcctggtcta caccaagaag caggcgttcc agctccgagc gcacatgtac caggcccgca
gcctctttgc cgccgacagc agcggactct cagacccctt tgcccgcgtc ttcttcatca
atcagagtca gtgcacagag gtgctgaatg agaccctgtg tcccacctgg gaccagatgc
tggtgttcga caacctggag ctctatggtg aagctcatga gctgagggac gatccgccca
tcattgtcat tgaaatctat gaccaggatt ccatgggcaa agctgacttc atgggccgga
ccttcgccaa acccctggtg aagatggcag acgaggcgta ctgcccaccc cgcttcccac
ctcagctcga gtactaccag atctaccgtg gcaacgccac agctggagac ctgctggcgg
ccttcgagct gctgcagatt ggaccagcag ggaaggctga cctgcccccc atcaatggcc
cggtggacgt ggaccgaggt cccatcatgc ccgtgcccat gggcatccgg cccgtgctca
gcaagtaccg agtggaggtg ctgttctggg gcctacggga cctaaagcgg gtgaacctgg
cccaggtgga ccggccacgg gtggacatcg agtgtgcagg gaagggggtg cagtcgtccc
tgatccacaa ttataagaag aaccccaact tcaacaccct cgtcaagtgg tttgaagtgg
acctcccaga gaacgagctg ctgcacccgc ccttgaacat ccgtgtggtg gactgccggg
ccttcggtcg ctacacactg gtgggctccc atgccgtcag ctccctgcga cgcttcatct
accggccccc agaccgctcg gcccccagct ggaacaccac ggtcaggctt ctccggcgct
gccgtgtgct gtgcaatggg ggctcctcct ctcactccac aggggaggtt gtggtgacta
tggagccaga ggtacccatc aagaaactgg agaccatggt gaagctggac gcgacttctg
aagctgttgt caaggtggat gtggctgagg aggagaagga gaagaagaag aagaagaagg
gcactgcgga ggagccagag gaggaggagc cagacgagag catgctggac tggtggtcca
agtactttgc ctccattgac accatgaagg agcaacttcg acaacaagag ccctctggaa
ttgacttgga ggagaaggag gaagtggaca ataccgaggg cctgaagggg tcaatgaagg
gcaaggagaa ggcaagggct gccaaagagg agaagaagaa gaaaactcag agctctggct
ctggccaggg gtccgaggcc cccgagaaga agaaacccaa gattgatgag cttaaggtat
accccaaaga gctggagtcc gagtttgata actttgagga ctggctgcac actttcaact
tgcttcgggg caagaccggg gatgatgagg atggctccac cgaggaggag cgcattgtgg
gacgcttcaa gggctccctc tgcgtgtaca aagtgccact cccagaggac gtgtcccggg
aagccggcta cgactccacc tacggcatgt tccagggcat cccgagcaat gaccccatca
atgtgctggt ccgagtctat gtggtccggg ccacggacct gcaccctgct gacatcaacg
gcaaagctga cccctacatc gccatccggc taggcaagac tgacatccgc gacaaggaga
actacatctc caagcagctc aaccctgtct ttgggaagtc ctttgacatc gaggcctcct
tccccatgga atccatgctg acggtggctg tgtatgactg ggacctggtg ggcactgatg
acctcattgg ggaaaccaag atcgacctgg agaaccgctt ctacagcaag caccgcgcca
cctgcggcat cgcccagacc tactccacac atggctacaa tatctggcgg gaccccatga
agcccagcca gatcctgacc cgcctctgca aagacggcaa agtggacggc ccccactttg
ggccccctgg gagagtgaag gtggccaacc gcgtcttcac tgggccctct gagattgagg
acgagaacgg tcagaggaag cccacagacg agcatgtggc gctgttggcc ctgaggcact
gggaggacat cccccgcgca ggctgccgcc tggtgccaga gcatgtggag acgaggccgc
tgctcaaccc cgacaagccg ggcatcgagc agggccgcct ggagctgtgg gtggacatgt
tccccatgga catgccagcc cctgggacgc ctctggacat ctcacctcgg aagcccaaga
agtacgagct gcgggtcatc atctggaaca cagatgaggt ggtcttggag gacgacgact
tcttcacagg ggagaagtcc agtgacatct tcgtgagggg gtggctgaag ggccagcagg
aggacaagca ggacacagac gtccactacc actccctcac tggcgagggc aacttcaact
ggcgctacct gttccccttc gactacctgg cggcggagga gaagatcgtc atctccaaga
aggagtccat gttctcctgg gacgagaccg agtacaagat ccccgcgcgg ctcaccctgc
agatctggga tgcggaccac ttctccgctg acgacttcct gggggccatc gagctggacc
tgaaccggtt cccgcggggc gcaaagacag ccaagcagtg caccatggag atggccaccg
gggaggtgga cgtgcccctc gtgtccatct tcaagcaaaa gcgcgtcaaa ggctggtggc
ccctcctggc ccgcaatgag aacgatgagt ttgagctcac gggcaaggtg gaggctgagc
tgcatttact gacagcagag gaggcagaga agaacccagt gggcctggcc cgcaatgaac
ctgaccccct agagaaaccc aaccggcccg acacgagctt catctggttc ctgaaccctc
tcaagtcggc tcgctacttc ttgtggcaca cgtatcgctg gctgctcctc aaactgttgc
tgctcctgct gctgctcctc ctcctcgccc tgttcctcta ctctgtgcct ggctacctgg
tcaagaaaat cctcggggcc tga
Human Otoferlin Transcript Variant 4 (encodes the protein of SEQ ID NO: 9)
(SEQ ID NO: 17)
atga tcaaaacgga gaagtcctac cctgagcgtc gcctgcgggg cgtcctggag gagctgagct
gtggctgctg ccgcttcctc tccctcgctg acaaggacca gggccactca tcccgcacca
ggcttgaccg ggagcgcctc aagtcctgca tgagggagct ggaaaacatg gggcagcagg
ccaggatgct gcgggcccag gtgaagcggc acacggtgcg ggacaagctg aggctgtgcc
agaacttcct gcagaagctg cgcttcctgg cggacgagcc ccagcacagc attcccgaca
tcttcatctg gatgatgagc aacaacaagc gtgtcgccta tgcccgtgtg ccctccaagg
acctgctctt ctccatcgtg gaggaggaga ctggcaagga ctgcgccaag gtcaagacgc
tcttccttaa gctgccaggg aagcggggct tcggctcggc aggctggaca gtgcaggcca
aggtggagct gtacctgtgg ctgggcctca gcaaacagcg caaggagttc ctgtgcggcc
tgccctgtgg cttccaggag gtcaaggcag cccagggcct gggcctgcat gccttcccac
ccgtcagcct ggtctacacc aagaagcagg cgttccagct ccgagcgcac atgtaccagg
cccgcagcct ctttgccgcc gacagcagcg gactctcaga cccctttgcc cgcgtcttct
tcatcaatca gagtcagtgc acagaggtgc tgaatgagac cctgtgtccc acctgggacc
agatgctggt gttcgacaac ctggagctct atggtgaagc tcatgagctg agggacgatc
cgcccatcat tgtcattgaa atctatgacc aggattccat gggcaaagct gacttcatgg
gccggacctt cgccaaaccc ctggtgaaga tggcagacga ggcgtactgc ccaccccgct
tcccacctca gctcgagtac taccagatct accgtggcaa cgccacagct ggagacctgc
tggcggcctt cgagctgctg cagattggac cagcagggaa ggctgacctg ccccccatca
atggcccggt ggacgtggac cgaggtccca tcatgcccgt gcccatgggc atccggcccg
tgctcagcaa gtaccgagtg gaggtgctgt tctggggcct acgggaccta aagcgggtga
acctggccca ggtggaccgg ccacgggtgg acatcgagtg tgcagggaag ggggtgcagt
cgtccctgat ccacaattat aagaagaacc ccaacttcaa caccctcgtc aagtggtttg
aagtggacct cccagagaac gagctgctgc acccgccctt gaacatccgt gtggtggact
gccgggcctt cggtcgctac acactggtgg gctcccatgc cgtcagctcc ctgcgacgct
tcatctaccg gcccccagac cgctcggccc ccagctggaa caccacgggg gaggttgtgg
tgactatgga gccagaggta cccatcaaga aactggagac catggtgaag ctggacgcga
cttctgaagc tgttgtcaag gtggatgtgg ctgaggagga gaaggagaag aagaagaaga
agaagggcac tgcggaggag ccagaggagg aggagccaga cgagagcatg ctggactggt
ggtccaagta ctttgcctcc attgacacca tgaaggagca acttcgacaa caagagccct
ctggaattga cttggaggag aaggaggaag tggacaatac cgagggcctg aaggggtcaa
tgaagggcaa ggagaaggca agggctgcca aagaggagaa gaagaagaaa actcagagct
ctggctctgg ccaggggtcc gaggcccccg agaagaagaa acccaagatt gatgagctta
aggtataccc caaagagctg gagtccgagt ttgataactt tgaggactgg ctgcacactt
tcaacttgct tcggggcaag accggggatg atgaggatgg ctccaccgag gaggagcgca
ttgtgggacg cttcaagggc tccctctgcg tgtacaaagt gccactccca gaggacgtgt
cccgggaagc cggctacgac tccacctacg gcatgttcca gggcatcccg agcaatgacc
ccatcaatgt gctggtccga gtctatgtgg tccgggccac ggacctgcac cctgctgaca
tcaacggcaa agctgacccc tacatcgcca tccggctagg caagactgac atccgcgaca
aggagaacta catctccaag cagctcaacc ctgtctttgg gaagtccttt gacatcgagg
cctccttccc catggaatcc atgctgacgg tggctgtgta tgactgggac ctggtgggca
ctgatgacct cattggggaa accaagatcg acctggagaa ccgcttctac agcaagcacc
gcgccacctg cggcatcgcc cagacctact ccacacatgg ctacaatatc tggcgggacc
ccatgaagcc cagccagatc ctgacccgcc tctgcaaaga cggcaaagtg gacggccccc
actttgggcc ccctgggaga gtgaaggtgg ccaaccgcgt cttcactggg ccctctgaga
ttgaggacga gaacggtcag aggaagccca cagacgagca tgtggcgctg ttggccctga
ggcactggga ggacatcccc cgcgcaggct gccgcctggt gccagagcat gtggagacga
ggccgctgct caaccccgac aagccgggca tcgagcaggg ccgcctggag ctgtgggtgg
acatgttccc catggacatg ccagcccctg ggacgcctct ggacatctca cctcggaagc
ccaagaagta cgagctgcgg gtcatcatct ggaacacaga tgaggtggtc ttggaggacg
acgacttctt cacaggggag aagtccagtg acatcttcgt gagggggtgg ctgaagggcc
agcaggagga caagcaggac acagacgtcc actaccactc cctcactggc gagggcaact
tcaactggcg ctacctgttc cccttcgact acctggcggc ggaggagaag atcgtcatct
ccaagaagga cctggcccgc aatgagaacg atgagtttga gctcacgggc aaggtggagg
ctgagctgca tttactgaca gcagaggagg cagagaagaa cccagtgggc ctggcccgca
atgaacctga ccccctagag aaacccaacc ggcccgacac ggccttcgtc tggttcctca
accctctcaa gtccatcaag tacctcatct gcacccggta caagtggctc atcatcaaga
tcgtgctggc gctgttgggg ctgctcatgt tggggctctt cctctacagc ctccctggct
acatggtcaa aaagctcctt ggggcatga
Human Otoferlin Transcript Variant 5 (encodes the protein of SEQ ID NO: 12)
(SEQ ID NO: 18)
atg gccttgctca tccacctcaa gacagtctcg gagctgcggg gcaggggcga ccggatcgcc
aaagtgactt tccgagggca atccttctac tctcgggtcc tggagaactg tgaggatgtg
gctgactttg atgagacatt tcggtggccg gtggccagca gcatcgacag aaatgagatg
ctggagattc aggttttcaa ctacagcaaa gtcttcagca acaagctcat cgggaccttc
cgcatggtgc tgcagaaggt ggtagaggag agccatgtgg aggtgactga cacgctgatt
gatgacaaca atgctatcat caagaccagc ctgtgcgtgg aggtccggta tcaggccact
gacggcacag tgggctcctg ggacgatggg gacttcctgg gagatgagtc tcttcaagag
gaagagaagg acagccaaga gacggatgga ctgctcccag gctcccggcc cagctcccgg
cccccaggag agaagagctt ccggagagcc gggaggagcg tgttctccgc catgaagctc
ggcaaaaacc ggtctcacaa ggaggagccc caaagaccag atgaaccggc ggtgctggag
atggaagacc ttgaccatct ggccattcgg ctaggagatg gactggatcc cgactcggtg
tctctagcct cagtcacagc tctcaccact aatgtctcca acaagcgatc taagccagac
attaagatgg agccaagtgc tgggcggccc atggattacc aggtcagcat cacggtgatc
gaggcccggc agctggtggg cttgaacatg gaccctgtgg tgtgcttgga ggtgggtgac
gacaagaagt acacatccat gaaggagtcc actaactgcc cctattacaa cgagtacttc
gtcttcgact tccatgtctc tccggatgtc atgtttgaca agatcatcaa gatttcggtg
attcactcca agaacctgct gcgcagtggc accctggtgg gctccttcaa aatggacgtg
ggaaccgtgt actcgcagcc agagcaccag ttccatcaca agtgggccat cctgtctgac
cccgatgaca tctcctcggg gctgaagggc tacgtgaagt gtgacgttgc cgtggtgggc
aaaggggaca acatcaagac gccccacaag gccaatgaga ccgacgaaga tgacattgag
gggaacttgc tgctccccga gggggtgccc cccgaacgcc agtgggcccg gttctatgtg
aaaatttacc gagcagaggg gctgccccgt atgaacacaa gcctcatggc caatgtaaag
aaggctttca tcggtgaaaa caaggacctc gtggacccct acgtgcaagt cttctttgct
ggccagaagg gcaagacttc agtgcagaag agcagctatg agcccctgtg gaatgagcag
gtcgtcttta cagacctctt ccccccactc tgcaaacgca tgaaggtgca gatccgagac
tcggacaagg tcaacgacgt ggccatcggc acccacttca ttgacctgcg caagatttct
aatgacggag acaaaggctt cctgcccaca ctgggcccag cctgggtgaa catgtacggc
tccacacgta actacacgct gctggatgag catcaggacc tgaacgaggg cctgggggag
ggtgtgtcct tccgggcccg gctcctgctg ggcctggctg tggagatcgt agacacctcc
aaccctgagc tcaccagctc cacagaggtg caggtggagc aggccacgcc catctcggag
agctgtgcag gtaaaatgga agaattcttt ctctttggag ccttcctgga ggcctcaatg
atcgaccgga gaaacggaga caagcccatc acctttgagg tcaccatagg caactatggg
aacgaagttg atggcctgtc ccggccccag cggcctcggc cccggaagga gccgggggat
gaggaagaag tagacctgat tcagaacgca agtgatgacg aggccggtga tgccggggac
ctggcctcag tctcctccac tccaccaatg cggccccagg tcaccgacag gaactacttc
catctgccct acctggagcg aaagccctgc atctacatca agagctggtg gccggaccag
cgccgccgcc tctacaatgc caacatcatg gaccacattg ccgacaagct ggaagaaggc
ctgaacgaca tacaggagat gatcaaaacg gagaagtcct accctgagcg tcgcctgcgg
ggcgtcctgg aggagctgag ctgtggctgc tgccgcttcc tctccctcgc tgacaaggac
cagggccact catcccgcac caggcttgac cgggagcgcc tcaagtcctg catgagggag
ctggaaaaca tggggcagca ggccaggatg ctgcgggccc aggtgaagcg gcacacggtg
cgggacaagc tgaggctgtg ccagaacttc ctgcagaagc tgcgcttcct ggcggacgag
ccccagcaca gcattcccga catcttcatc tggatgatga gcaacaacaa gcgtgtcgcc
tatgcccgtg tgccctccaa ggacctgctc ttctccatcg tggaggagga gactggcaag
gactgcgcca aggtcaagac gctcttcctt aagctgccag ggaagcgggg cttcggctcg
gcaggctgga cagtgcaggc caaggtggag ctgtacctgt ggctgggcct cagcaaacag
cgcaaggagt tcctgtgcgg cctgccctgt ggcttccagg aggtcaaggc agcccagggc
ctgggcctgc atgccttccc acccgtcagc ctggtctaca ccaagaagca ggcgttccag
ctccgagcgc acatgtacca ggcccgcagc ctctttgccg ccgacagcag cggactctca
gacccctttg cccgcgtctt cttcatcaat cagagtcagt gcacagaggt gctgaatgag
accctgtgtc ccacctggga ccagatgctg gtgttcgaca acctggagct ctatggtgaa
gctcatgagc tgagggacga tccgcccatc attgtcattg aaatctatga ccaggattcc
atgggcaaag ctgacttcat gggccggacc ttcgccaaac ccctggtgaa gatggcagac
gaggcgtact gcccaccccg cttcccacct cagctcgagt actaccagat ctaccgtggc
aacgccacag ctggagacct gctggcggcc ttcgagctgc tgcagattgg accagcaggg
aaggctgacc tgccccccat caatggcccg gtggacgtgg accgaggtcc catcatgccc
gtgcccatgg gcatccggcc cgtgctcagc aagtaccgag tggaggtgct gttctggggc
ctacgggacc taaagcgggt gaacctggcc caggtggacc ggccacgggt ggacatcgag
tgtgcaggga agggggtgca gtcttccctg atccacaatt ataagaagaa ccccaacttc
aacaccctcg tcaagtggtt tgaagtggac ctcccagaga acgagctgct gcacccgccc
ttgaacatcc gtgtggtgga ctgccgggcc ttcggtcgct acacactggt gggctcccat
gccgtcagct ccctgcgacg cttcatctac cggcccccag accgctcggc ccccagctgg
aacaccacgg tcaggcttct ccggcgctgc cgtgtgctgt gcaatggggg ctcctcctct
cactccacag gggaggttgt ggtgactatg gagccagagg tacccatcaa gaaactggag
accatggtga agctggacgc gacttctgaa gctgttgtca aggtggatgt ggctgaggag
gagaaggaga agaagaagaa gaagaagggc actgcggagg agccagagga ggaggagcca
gacgagagca tgctggactg gtggtccaag tactttgcct ccattgacac catgaaggag
caacttcgac aacaagagcc ctctggaatt gacttggagg agaaggagga agtggacaat
accgagggcc tgaaggggtc aatgaagggc aaggagaagg caagggctgc caaagaggag
aagaagaaga aaactcagag ctctggctct ggccaggggt ccgaggcccc cgagaagaag
aaacccaaga ttgatgagct taaggtatac cccaaagagc tggagtccga gtttgataac
tttgaggact ggctgcacac tttcaacttg cttcggggca agaccgggga tgatgaggat
ggctccaccg aggaggagcg cattgtggga cgcttcaagg gctccctctg cgtgtacaaa
gtgccactcc cagaggacgt gtcccgggaa gccggctacg actccaccta cggcatgttc
cagggcatcc cgagcaatga ccccatcaat gtgctggtcc gagtctatgt ggtccgggcc
acggacctgc accctgctga catcaacggc aaagctgacc cctacatcgc catccggcta
ggcaagactg acatccgcga caaggagaac tacatctcca agcagctcaa ccctgtcttt
gggaagtcct ttgacatcga ggcctccttc cccatggaat ccatgctgac ggtggctgtg
tatgactggg acctggtggg cactgatgac ctcattgggg aaaccaagat cgacctggag
aaccgcttct acagcaagca ccgcgccacc tgcggcatcg cccagaccta ctccacacat
ggctacaata tctggcggga ccccatgaag cccagccaga tcctgacccg cctctgcaaa
gacggcaaag tggacggccc ccactttggg ccccctggga gagtgaaggt ggccaaccgc
gtcttcactg ggccctctga gattgaggac gagaacggtc agaggaagcc cacagacgag
catgtggcgc tgttggccct gaggcactgg gaggacatcc cccgcgcagg ctgccgcctg
gtgccagagc atgtggagac gaggccgctg ctcaaccccg acaagccggg catcgagcag
ggccgcctgg agctgtgggt ggacatgttc cccatggaca tgccagcccc tgggacgcct
ctggacatct cacctcggaa gcccaagaag tacgagctgc gggtcatcat ctggaacaca
gatgaggtgg tcttggagga cgacgacttc ttcacagggg agaagtccag tgacatcttc
gtgagggggt ggctgaaggg ccagcaggag gacaagcagg acacagacgt ccactaccac
tccctcactg gcgagggcaa cttcaactgg cgctacctgt tccccttcga ctacctggcg
gcggaggaga agatcgtcat ctccaagaag gagtccatgt tctcctggga cgagaccgag
tacaagatcc ccgcgcggct caccctgcag atctgggatg cggaccactt ctccgctgac
gacttcctgg gggccatcga gctggacctg aaccggttcc cgcggggcgc aaagacagcc
aagcagtgca ccatggagat ggccaccggg gaggtggacg tgcccctcgt gtccatcttc
aagcaaaagc gcgtcaaagg ctggtggccc ctcctggccc gcaatgagaa cgatgagttt
gagctcacgg gcaaggtgga ggctgagctg catttactga cagcagagga ggcagagaag
aacccagtgg gcctggcccg caatgaacct gaccccctag agaaacccaa ccggcccgac
acggccttcg tctggttcct caaccctctc aagtccatca agtacctcat ctgcacccgg
tacaagtggc tcatcatcaa gatcgtgctg gcgctgttgg ggctgctcat gttggggctc
ttcctctaca gcctccctgg ctacatggtc aaaaagctcc ttggggcatg a

A non-limiting example of a human wildtype otoferlin genomic DNA sequence is SEQ ID NO: 19. The exons in SEQ ID NO: 19 are: nucleotide positions 5001-5206 (exon 1), nucleotide positions 25925-25983 (exon 2), nucleotide positions 35779-35867 (exon 3), nucleotide positions 44590-44689 (exon 4), nucleotide positions 47100-47281 (exon 5), nucleotide positions 59854-59927 (exon 6), nucleotide positions 61273-61399 (exon 7), nucleotide positions 61891-61945 (exon 8), nucleotide positions 68626-68757 (exon 9), nucleotide positions 73959-74021 (exon 10), nucleotide positions 74404-74488 (exon 11), nucleotide positions 79066-79225 (exon 12), nucleotide positions 80051-80237 (exon 13), nucleotide positions 81107-81293 (exon 14), nucleotide positions 82690-82913 (exon 15), nucleotide positions 83388-83496 (exon 16), nucleotide positions 84046-84226 (exon 17), nucleotide positions 84315-84435 (exon 18), nucleotide positions 85950-86050 (exon 19), nucleotide positions 86193-86283 (exon 20), nucleotide positions 86411-86527 (exon 21), nucleotide positions 86656-86808 (exon 22), nucleotide positions 87382-87571 (exon 23), nucleotide positions 87661-87785 (exon 24), nucleotide positions 88206-88340 (exon 25), nucleotide positions 89025-89186 (exon 26), nucleotide positions 89589-89708 (exon 27), nucleotide positions 90132-90293 (exon 28), nucleotide positions 90405-90567 (exon 29), nucleotide positions 91050-91180 (exon 30), nucleotide positions 92549-92578 (exon 31), nucleotide positions 92978-93106 (exon 32), nucleotide positions 95225-95291 (exon 33), nucleotide positions 96198-96334 (exon 34), nucleotide positions 96466-96600 (exon 35), nucleotide positions 96848-96985 (exon 36), nucleotide positions 97623-97750 (exon 37), nucleotide positions 97857-98027 (exon 38), nucleotide positions 98670-98830 (exon 39), nucleotide positions 99593-99735 (exon 40), nucleotide positions 100128-100216 (exon 41), nucleotide positions 101518-101616 (exon 42), nucleotide positions 101762-102003 (exon 43), nucleotide positions 102669-102847 (exon 44), nucleotide positions 102952-103052 (exon 45), nucleotide positions 103494-103691 (exon 46), nucleotide positions 105479-106496 (exon 47), and exon 48 (the sequence starting with CCGGCCCGAC (SEQ ID NO: 89); see also the description of this exon in Yasunaga et al., Am. J. Hum. Genet. 67:591-600, 2000).

The introns are located between each contiguous pair of exons in SEQ ID NO: 19, i.e., at nucleotide positions 100-5001 (intron 1), nucleotide 5207-25924 (intron 2), nucleotide positions 25984-35778 (intron 3), nucleotide positions 35868-44589 (intron 4), nucleotide positions 44690-47099 (intron 5), nucleotide positions 47282-59853 (intron 6), nucleotide positions 59928-61272 (intron 7), nucleotide positions 61400-61890 (intron 8), nucleotide positions 61946-68625 (intron 9), nucleotide positions 68758-73958 (intron 10), nucleotide positions 74022-74403 (intron 11), nucleotide positions 74489-79065 (intron 12), nucleotide positions 79226-80050 (intron 13), nucleotide positions 80238-81106 (intron 14), nucleotide positions 81294-82689 (intron 15), nucleotide positions 82914-83387 (intron 16), nucleotide positions 83497-84045 (intron 17), nucleotide positions 84227-84314 (intron 18), nucleotide positions 84436-85949 (intron 19), nucleotide positions 86051-86192 (intron 20), nucleotide positions 86284-86410 (intron 21), nucleotide positions 86528-86655 (intron 22), nucleotide positions 86809-87381 (intron 23), nucleotide positions 87572-87660 (intron 24), nucleotide positions 87786-88205 (intron 25), nucleotide positions 88341-89024 (intron 26), nucleotide positions 89187-89588 (intron 27), nucleotide positions 89709-90131 (intron 28), nucleotide positions 90294-90404 (intron 29), nucleotide positions 90568-91049 (intron 30), nucleotide positions 91181-92548 (intron 31), nucleotide positions 92579-92977 (intron 32), nucleotide positions 93107-95224 (intron 33), nucleotide positions 95292-96197 (intron 34), nucleotide positions 96335-96465 (intron 35), nucleotide positions 96601-96847 (intron 36), nucleotide positions 96986-97622 (intron 37), nucleotide positions 97751-97856 (intron 38), nucleotide positions 98028-98669 (intron 39), nucleotide positions 98831-99592 (intron 40), nucleotide positions 99736-100127 (intron 41), nucleotide positions 100217-101517 (intron 42), nucleotide positions 101617-101761 (intron 43), nucleotide positions 102004-102668 (intron 44), nucleotide positions 102848-102951 (intron 45), nucleotide positions 103053-103494 (intron 46), nucleotide positions 103692-105478 (intron 47), and nucleotide positions 106497-108496 (intron 48).

| Human Otoferlin Gene Sequence (NCBI Accession No. NG_009937.1) | |
|---|---|
| (SEQ ID NO: 19) | |
| 1 | gggagagcat gcgggctttt tcacccattt ggagactctg cctgcagaat cctcaagaca |
| 61 | ttcatttcat tcattcgttc actctttttt tcagacaagg gttcttagga tcattcattc |
| 121 | attctataca gatttgctga tactgtacag gttgcctctc agaatccagt gcctgttctc |
| 181 | agtacctatg ctgactggta ggagggataa agacagcctt acaccctggg agaggcggtc |
| 241 | cagagtagaa acatcagaca ctgtaggaga aagagccaaa gaacaggagg caacagccag |
| 301 | acttgcatgt tagatggttg gagtctgaaa atatccagag taaagaaagg taggtgtaac |
| 361 | ctagtatttg aaagtgctaa acagcaagcc agcgctcagg gcaagtggcc aggaagcttt |
| 421 | ccaagaaaaa cttctgggag gataaaggga ggtctgtgaa aggaccgacc gcaaagtcgg |
| 481 | gcgggtactg cacacatgac ccattaacta cggctcttcc ggccttgcca ggttcttcga |
| 541 | gcgggcaccc acccggtatc cttccctcct tcaaccaaca agtactgacc ttgcccgaat |
| 601 | gctgggtgct ggccctgtgc gccggacgga agggcctgat tttctcgcat tttctctaag |
| 661 | cgctggaggc tgcccgcctg gggcgtctcc aagcactaaa ccccacgtgg ctccatctcc |
| 721 | tcccccttcc ccggctacat ccatgcgagg tcccttatgc cctgctggct gtgccccact |
| 781 | tgccagggag cgccttcctg tgaaggggaa tctggggagg cccccgcccg tgcctgccca |
| 841 | cttcctactc tgaaaccaca aacctctagg aaggcccctc gccccgtcgg gtggaaaatt |
| 901 | ggctcctcgg ggttccttca accccaaagt ccgggggcag ggggctgaat ttcgatcacc |
| 961 | gaggagggcg ctagtggaag ggggagggga ggaggcgctc cctcggcctt acccgggcat |
| 1021 | gagtccaggg ggcacgtagg cggcattgaa ctcggtcagt agggtgcccg cgctgcggga |
| 1081 | ggccatggtg ggtgcgcgag tacagcgagc ctggcgctcc gctccgcccc acggtaaaca |
| 1141 | gcggcgggtc acctggcaac ggccacggcg gcgagcctgg caacgccagc tggccccgcg |
| 1201 | gtcctcgcgt tccgagacac tgcgcccccc tggcggccac ctcccggagc agccccatag |
| 1261 | ggcgcgccca gaaggcgcct cccatttccg cgagctctgc tgggtgaccc ctgtcacttg |
| 1321 | ccccggcgcg tcggggctct ctgccttgct gcctgcggga tgtgcagagg ctcagatgcg |
| 1381 | ggagggaggc agaacatcgc tggagaggcc tggcgggatc cgtcttgcgg tgacccggct |
| 1441 | tggaacgcga cgctaccaca aaggacaggc caggtcctga ctttggaggt cacgcacgaa |
| 1501 | gcagctgccc caccgtggct caggcccaga aacctgagtg gccttagggt ccccaggagc |
| 1561 | tatgtctctc caaggcttcc cctgcacccc agctccatgg gagttccagg aaggggctct |
| 1621 | gtagtgggcc gcgctgggcg aggttgccta ggacgctgtg tgtaattgtg tgtgtgcgtg |
| 1681 | cgtgcgtgca tgtgtgcgtg tgtgtgtgcg cgccttgcga agaggagcat ttttcatttt |
| 1741 | tcttcttaat ctctagctga tggcttcctt ctcacctcat catacctgta aactactcca |
| 1801 | caagaggcaa aaagaaaatc atagaacgtc tgggtgtgtg atttacattt gtccccaatg |
| 1861 | ctgccgttca ttttggggat ggcatccgca ggggtagcgc ttgccccaga gagcaggcct |
| 1921 | gctgaaggcc caccccctgt gtaatcctcc ctgtgactgc ttcagcctcc acatttgagt |
| 1981 | ttctccaggg acccttacac cagccttggt gcctagggca ggcacagggc cttcggcttt |
| 2041 | tccataaggg gtgagggtat ggggacagct tccacggtgc caggctcttg gtgactcggg |
| 2101 | tggaggccct gccaccccag ctccctctgc agcagtcctg gtgtcatgaa gaagcctggc |
| 2161 | agttcccact ttgtctccag gtcttgcttc ttcccttcag atgccggatg gagagcagtg |
| 2221 | ctcccctcgc acttcggttc tgtaaatggc agtggatgaa gaccgcagac gctactgggt |
| 2281 | tcacagaatg tggctgccac cagcattctc caaagacttc ccctggcaaa tgccaatgaa |
| 2341 | atgaagggga gccacaggct tcccagaggc tcaggaagca gaaacactga ggcctggttg |
| 2401 | gaaagagagc tgtaggcaaa ggaacggctg caaacaaact gggtggaagg aaaatccatc |
| 2461 | catccttcag aatgcagtca tgtctgtatt tttctaaatt ctcattgggg aacatgttct |
| 2521 | acaataagga aaacatgagt caaagacccc cagccctggt gaggttgacc gcctacttcc |
| 2581 | tcccccattc agggctgccc agccagtcct gctggtaaac ccaccaccct gccttccacc |
| 2641 | tgcctgtgtt ctagtccatc tgtctcgtct gattgggatc ctgtgccatc catatcatat |
| 2701 | tcatccctga atctcagcac ctggctctgg aaagaattca gtaaatgttc attacataaa |
| 2761 | ttatatactc agttaccaaa atgggtaaca gaagaatgga aattaaaaag tgggttctgg |
| 2821 | ctgggaacgg tggctcatgc ctgtaatccc agcattttgg aaggccgagg caggcggatc |
| 2881 | acttgaggtc agcagtttga gaccagccta gccaacatgg tgaaacccca tctctactaa |
| 2941 | aaatacaaac attagctggg catggtagca catgcttata gtctgaccta tttgggaggc |
| 3001 | tgaggcagga gaatcgcttg aacccgggag gcagaggttg cagtgagcct agatcactcc |
| 3061 | actgccctcc agcctgggtg acagagtgag actctctctc aaaaaaaaaa aaaaaaaaag |
| 3121 | agtgggatct gtctcttagg ttttcagaaa ggccagctga ctccattgag agctgctgag |
| 3181 | tggaattgct accattttat ggatgtggaa agtgagattc agcatagatc aatgccttgg |
| 3241 | ctagggcagg atcatgggct ttttcctaaa gatgcacagg gtctataaga ggcctcatga |
| 3301 | aattcactcc tttcagggga ggacctccag agaaccaagg aagaagatat tttctgttgt |
| 3361 | tgttgttgta ccttgtagtc accaagcctt aaaaacacag atcacctgat gacctcatgg |
| 3421 | atggaaaatg ctagtgaatt taggtgtttt tttttttaag acattaaact tatctagctt |
| 3481 | ttgaaaattt agcattcata cttgtattca ataaacagaa tactctaaga catttttaaa |
| 3541 | ttcaatatga ttagataaat gtgagaaagg taagcaagaa ttggttagga aaaagtgcaa |
| 3601 | aggaaaaaaa cgtaagtaga aaaagaaaaa tagggcacag tagcaaggaa atatctagaa |
| 3661 | ggaggaaaga aggctcttgg gacatactgt aggttatttg gaatgcagaa ttcaaaagaa |
| 3721 | gtaacctctt gttaccatgc tgtgaaaaat tagtatttct ctttttaacc tttagactct |
| 3781 | gtttcaggta gatccctgag ctcactcttt tatcctttca acagtaattg ggcatcctac |
| 3841 | ggtgcacagg ataccatgtg gggctctggg gtagagggat gctgtgctcc agggccctcc |
| 3901 | tgaggtcagg aggggaaaag gcaagggaag cagccccagg actcagtaag atgagaggag |
| 3961 | gcaggagcag cacagaggag gaccccaacc cagctggggt gcgggtacag aggcacctcc |
| 4021 | aggagcaggc actcaagctg agtccagggt ggggtggagg gcattccacc aagaatgaat |
| 4081 | agcaagaaca aggctgcaga tgaccctgca cagattccag catcagggat tctggccccc |
| 4141 | agcgtccctt tccttggtgt atgctgctgg aagacaggag ggggcagttc tcttttggtc |
| 4201 | cttagaaatt tccctataga catcccttct cctgccccta gcagcttata acagcagaag |
| 4261 | gccattcaat tctgctcctc tggctggaaa gcaaacatca gcctggctta ccttcgctag |
| 4321 | cctgcctgac ccccagcctt ggggtggggc taggttagtg ccctggatcc ttaaacatgg |
| 4381 | cccctggtag cacataagcc tctgctgcct ccacaaccag ccatctccat catgcatggc |
| 4441 | ctggtcctga gcctcccctc aggcagcagc cccagcctct ctgcttcctg agattcagaa |
| 4501 | acagggatcc cagggcaggg ctcagggccc agcctcttgg gggcacagtc tccagcaggg |
| 4561 | aagaggtggg cgtaggtgga caaatacatt cccttcttct tgtagtgtgc ccctgtgggt |
| 4621 | gggagtgccc cccgccccac atccacccta cctatgctca tgcccactcc tctgtgccca |
| 4681 | ctactggtgg cagggcttgc ttagcaggga agcagcagag ccctgcagag gggtgggcag |
| 4741 | agcctccctt ccaggagctg ggagaagaca cctgaagctg gacagagctt tatctgggag |
| 4801 | ccggcggctg ctgaataatg aattccaact ctgccgcact cccaggctgc acattgggtg |
| 4861 | cacacacaca cacacacaca cgcacacacc gatacacaca cgctgggctg acaacttgga |
| 4921 | ggggcggggg ctgtgaggct ctgtgagtcg ctgcaggagc agcttagctc ggcttggttg |
| 4981 | ccttggtctc tgtgggcagc atcggagggg ggtcgggagg aggaggagga ggcagcggca |
| 5041 | gagaagagag aggcgtgtga gccgtgctcc accggctagc tccttcccgc tgctcctgcc |
| 5101 | tggcagtgcc aggcagccca caccagcatg gccttgctca tccacctcaa gacagtctcg |
| 5161 | gagctgcggg gcaggggcga ccggatcgcc aaagtgactt tccgaggtag ggaagccgct |
| 5221 | gtctcagagg gacgccagct ctgagagctg gaccagccca tgctctggag gtggccgggg |
| 5281 | ataggacagg gctgggagga tgggtgggaa aagttctagc aggggctggg gcgacgagcc |
| 5341 | ttttcacagg tagtcatctg cctgggactg actggccatt gtgatttctg gaaaatcagg |
| 5401 | gtcccctgtt cttccatgcc tgtgtctcct tgtgcttgcc aacatgccag ttccccgggc |
| 5461 | agggcacaga gccctctgcc tggggaggca gaagtgagga ccacatccct gagctcaggg |
| 5521 | agacccagga ccccctcctt ctctctctct ccctctcctg caggttgatt tccttgagca |
| 5581 | cagggtgggg gccgggtatg gagcctgggg cacccagggt gactgggaga aaccaggcag |
| 5641 | ccagtagctc cctgggcccc agagtgatgg gggacactgt tggcttcttt tagatctatt |
| 5701 | catgggcggg aaccccggga gcccctcgcc tctcttctgt ctggtctttt ctcatccttt |
| 5761 | tcactcctgt attcagcgac catttattat gatctactgt gtcccaagga taggggtaga |
| 5821 | aacacagagc tgagcacaac ccagccccag ccccaaggag cctgcatctg ttgggcaggc |
| 5881 | gtggaacaag caggacaggt atgtgagaca gaaggtgggg gtgcagcatg caagctacag |
| 5941 | aagcagcacc ctcacccagg ctggtgaccg gtggtcaggg aaggcttccc ggaggaagta |
| 6001 | acactcagct aatacccgaa ggaccagcgg gtgggagcta ggagaggagg taaggactga |
| 6061 | ggaggcagag gaaacagtgt gcccagagct gagagaggag ctgaaactct ggaactgaaa |
| 6121 | gaggttccat gtgtctacag ctcagagagg gtgcaggagc ggaggccaga gagccaagca |
| 6181 | gggcaaggag gcgaggctgg acctgacccc ggggacagcg gagcccctga gcattttaag |
| 6241 | tggggaaagg cagggccagg ctgactctgt ccttcaccac ccacctctgc ctctccatgt |
| 6301 | ggttgctgcc cagccctgag ggttgggggt acttctcact agacatggcc ccttctcccg |
| 6361 | agagaggggt ctgagagtgg gatggggacg aggtggtgag aagggggcag agaggacccc |
| 6421 | cctatactaa agagcaaagt gagccttggg gcccaggctg gacacagtgt ctgcacctct |
| 6481 | gttggaaggg atgggagaga ccattggcag aacattccag caagagccct ggggcaccca |
| 6541 | catcacctac ccagaggccc tgggatgccc acattgcagc ttgtgttcat agggacaggc |
| 6601 | aggggtggag gtgatggcag tggccctgcc ggcaggcgga ggttaggcag gagggagagg |
| 6661 | gaggtgtggg ctgggctgcc ttttgcctcc caccccaacc cactgactgc tttctctgca |
| 6721 | gcaccctgga tctccctctg tgtgccacct gcaggctcat gctggcttta gaaaaagcaa |
| 6781 | atgtgtcctt tcatctgtga aatgtggccc tgagctccac gggcagcata gtgtggggcg |
| 6841 | gttaagctac aggttgccat cctgctgcag ctgtgctgga acggtccagg ctgcaccgga |
| 6901 | caggcccact tgcaggccga gaaaccaagg ttcaggggta gaacactgcg cgtggcattc |
| 6961 | acggaggagc tgaggcacgg tggtcccagc tctcaggacc tcatcctgag tactcaatct |
| 7021 | tgcaagctct ttatctgctc ctgggccacc tgggggccag caccttgtct gacacccatc |
| 7081 | ttctccttgg agcagtgaag ggctgggcct ttagcctcac tgaccacaca ggaattagtt |
| 7141 | tatttgcaac ccttgctctc tggactgagt ggagaaatcg cttccaagct ccacattggc |
| 7201 | aggaggtggc catgggcaga gctcctgggg aagggggctg ggcagcagga ataggcagat |
| 7261 | gacaagcagt ttccagaaag tacctttatt acaacgaaag atgcctcagc cgctcacctg |
| 7321 | tgtcatctag cagttcacag ttgtaggcgc cccaaaagac caacactgac cacaattcag |
| 7381 | agggaatgga cccgctctac aatgttcctt agcatctccc agcacatgca accactgggg |
| 7441 | gccaagcaag tcctctgcag ggggaaacaa gaaactggta aacacgcaga gggagacact |
| 7501 | ggccccaggc tccccaaggc ttttgagtga aagtcaatgc cgggggaggt ggtgagggag |
| 7561 | cccttggtga gctgtgccca ttggcagtgg ccctaggtag gccagagcag ctggttgaaa |
| 7621 | tatggcttga ccttctgtgc tgtgccatcc aagacactaa ggaaagagcc ctcctgggct |
| 7681 | attgtggaaa aagttctgga tatggagtca gaagccctag atcctacctg ggttctgcca |
| 7741 | ctcaccctca gtttcctcat ctgtaaaatg ggcatgataa taacactcac ttcactagat |
| 7801 | tgcttcaggg aaaccatagg tatggaagag tttgtgggaa gagtattcaa atggtcgatg |
| 7861 | tgtttgtgag gcagccattc ccattctcct tgggagcagc tgctggcccc tgaggtatct |
| 7921 | tctctccaga ccagcagagg gccccatgtg agatataaag ggaagaggag agcacgtcta |
| 7981 | aggggttcca gtctgccagg agaaggcaga gagcctggac tctgctctgt aggagcctct |
| 8041 | gctgagatgt tccgcaggaa cccaggcagg cagacccccg agactcctgt tctcatctgg |
| 8101 | ctgggtgtgc accagctttg cccgtggcca cctcttttcc cactgccctc aggccagtcg |
| 8161 | gcccctcggt cagtgctccc agccagttct gtatctagct cttagttctt actgcccctg |
| 8221 | gctgctccta agctggtgct gccctgttca tgcatttcct gaacatgcta atatttgtgc |
| 8281 | acaggcctgg gaccctgagg tgaaggggcc agcctggccc tccaggagtc acaatcccat |
| 8341 | tgggtcagac aggagatagg gagtcctggg cgaatctcta ttgtgcctgc aggccacagg |
| 8401 | gctcagctgg gtttctcatt aacaaaatgg agacagtggt cctcatttag ctcagaggcc |
| 8461 | tgcagttccc aggagtaatc aagagtgggc tatctaagaa ggagccatct tgaccacagg |
| 8521 | ttgctactca aagagaggaa gtattagctt caggtatttc ctttatcaac tcctgagata |
| 8581 | gttccctaca tcaatcacag agccacagaa cgcagggtgt gaagggtccc gaagttcttc |
| 8641 | tagtccaacc ttctgtccag tgctgagcac caaggctggg ggacctggac tgtaagggaa |
| 8701 | ccaagtgaca gacagtccag ggccaaccct gatgtctccc ttctctttct atcccctgag |
| 8761 | ccttcttgcc aagacatgag gtttggggat tttctgtgtg agataagaac atctaaaggc |
| 8821 | tggatctggc ttccacataa ttcactgtgg ctggaaattg gcacaaaggt ttgcagggca |
| 8881 | tccccctcca ccttttcatt cttactcttc ttcccaccct ccccctgatt cccaccctgc |
| 8941 | tatgttccca ctacattcaa cttccagcca ttcccaaaag ataccacacc cttttacacc |
| 9001 | tccaaacttt gagacatcga tcatatttct tctgcctgga atgaccaccc tccaccttga |
| 9061 | gaacgcccat taattctgct catccttcaa ggcccagctc atatgctacc tcctccatga |
| 9121 | aggagtagta ctttgcacac aaagtactcc tctagcagta ctttgcacac attcttccta |
| 9181 | ctgctctgcc aaggtacatt gcatttcaga aactggccac acctctgcat gctgcccaag |
| 9241 | cttttgagca tcttcaggac agacatcaga catggtttct gtctttgagg gattcactgt |
| 9301 | gtcatgggtt tgagttcttc cactgtggca ctgtcactct ttggctccat ttccccaact |
| 9361 | gtgagctctc accccagaac tttctatcca aggaggatca agggactgga agaggcctca |
| 9421 | agggccaaga accaagaacc ttggctctta ctccttccat caacttccct aacccagaga |
| 9481 | atcttctagg gctccagagg ggcctaacga tttttttttt tttttttttt tttgagtctc |
| 9541 | gctctgtcgc caggctggag tgcagtgcag tgcgatctcg gctcactgca acctccagct |
| 9601 | cctgggttca agcgattctt ctgcctcagc ctctggagta gctgggacta caggcacacg |
| 9661 | ccaccatgcc cgactaattt ttgtattttt agtagagacg gtgtttcacc atatttccca |
| 9721 | ggctggtctc aaactcctga cctcatgatc cgcctgcctc ggcctcccaa agtgctggga |
| 9781 | ttacaggtgt gagccactgc gcccagtcaa gcctaacaaa tattagcaag ttcagctcca |
| 9841 | gcccataggt gggaagcaag agggattgcg tgtgtaagcc agagtcagtt aggggaagac |
| 9901 | agaggaagga gaaagggcag acgggaaagg aggtgggagc aggctctgag ttggcccccg |
| 9961 | gagctcagaa gggaggcggg agtttttatt gagcagggga aatgggcggt atttaagaag |
| 10021 | ccaccgaagg gtcttagttc attggcagga tcaaaggcca tgaaagggcc aggcgtggtg |
| 10081 | gctcatgcct ctttccgagc cttactgcga ccacacgtgg ggcaggatct caccatctga |
| 10141 | cagaggagaa gaaacataca cacagaggtg aaatgagtgc agcatacagg agggccagcg |
| 10201 | gtgggggtaa atagcaacaa tgttcagagg atgacagctt acttagagct acagagacga |
| 10261 | gggaaaagga gcaggaggag gaagtgccta agctgggtct tgaaaaactg cccctgtttg |
| 10321 | tacctatgga gatgcaggaa gtgaggaatt ggtgatggag gaaggtgaag tctttttggg |
| 10381 | tgagggaccg accagaccag aggccagagg aggagaattg cagccaagtt ccatgtcggg |
| 10441 | gaagccacac catgtagctg gagcagaggc tataggaagt agaagaggga gaccagctag |
| 10501 | ggagggagca gtgggcctgc tgagtgattg tgcagggaag ggttgcagct gaggcactgg |
| 10561 | ggacaagatg tccccctact gttggctgca ggctccacct ctgactccac attacattta |
| 10621 | ttttccccag taaaatgagg gagccctccc tcttgatgat aaatatagct actatttgga |
| 10681 | aacttatctg ccgcccctgg taaaggaggc agaataccca gtgcacccac ccctgaaatg |
| 10741 | ctcctgcaat gcctaggcct gtctgtgggc tgtggtccag cctagcaccc cctctcttgg |
| 10801 | agaatgtgag gttggcattg tccctgctga aggcagcaat agaggggacc cttacgacga |
| 10861 | ccttggcagc cggtggctgg ctgtggacac agccctgcgc ttttgtttta gtcctaacgg |
| 10921 | atatgccccc tgcatcctcc attcttcaca gcacactcag tccccctcac cacacctgag |
| 10981 | ggaaatcttc tctgcctaat gccccacgta gtgaatgttc tcccctcctc cctcccccag |
| 11041 | agcaagccat tgcacaccag cgaagggcag acagagcaca cagaaaattc cccagacttc |
| 11101 | tgctttgata ggaagtgtat tttcatgtaa tggaaatgat tgtaaaactg ccttcccctg |
| 11161 | caaaagccct caggaatatg ctgctggaaa tgtgcttgag aacagacttg caaatcattt |
| 11221 | ctctgaattg ggaagtcttg gttatcttag ccacttttac cgatcatgtg aaagccacat |
| 11281 | ctctcgcacc cactcccatc cctgggtctc atttttttct tttttctgtc gcccacgctg |
| 11341 | gagtgcagtg gcacaatcat agttcactgc aacctcaacc tcctgggttc aagcgatgct |
| 11401 | cccacctcag cctcccgagt agctaggact acaggcacgc accaccatgc tcagctaatt |
| 11461 | tttgtatttt tagtagagat ggggtttcac catattgccc aggctggtct tgaactcccg |
| 11521 | ggctcatgtg agctgcctgg ctcagcctcc caaagtgctg ggattatagg cgtaagccac |
| 11581 | tgtgcctggc cttttttttt tttttttaac tttttatata taaaaaaact ctcaaataca |
| 11641 | tacaagagtg gggagaacag tatcatggcc ctatgttgta ccatccaatt tcagctacca |
| 11701 | tcaattcatg gccaatcttg cttcatttgc atcccatcct acttcctcac cccaaattat |
| 11761 | tttgaagaag atcccaaacc ccataccatt ccatctgtaa atattcagta cacagttata |
| 11821 | aaagataaga gctcaaataa ataccacata ctttgtcatt ttaaagaaat taataacaat |
| 11881 | tccttagtac tataaactaa tcaggcagtc atcaaattcc taattgtctc atcaatgtca |
| 11941 | taattttttt ccaatttatt aaaagcttca ttttaatgtg ttttgtaaat caggtattta |
| 12001 | atcatttatc cactcgttct gttgaagggc agccatgtgc cacgcactgg gggatggtgg |
| 12061 | ggaatgagac agatgtggtt cctgtcctca aagggggaag atgaccactg cacccatcat |
| 12121 | catgaggagc tttatggaag ggaggcacag gtgcccaggc agctcatggt gaggaaaccc |
| 12181 | acttagccca ggatgggggg aagccacccc agtgcgtgag ccagagtcag ttaggggaag |
| 12241 | actgaggaag gaggaagggc agatgggaag ggagggagga gcaggctcct agttggcccc |
| 12301 | tagagcccgg aagtgagtca ggagttttta ttgagcagga ggaatgggcg gtatttaaga |
| 12361 | agcccttgaa gggtcgcagc tcattggcag gaacaaaggc catgaaaggg ctgggcatgg |
| 12421 | tggctcacgc ctgtaatccc agcactttgg gaggccaagg cgggggggat catttgaggg |
| 12481 | caggagtttg agaccagcct ggccaacatg gtgaaaccac atctctacta aaaacacaaa |
| 12541 | aattagccgg gcgtggtggt gcacgcctat aatcccagct actcgggagg ctgaggcagg |
| 12601 | agaatcgctt gaacccagga ggcagaggct gcagtgagcc gagattgtgc cactgcactc |
| 12661 | cagcctgggc gacagagcga gactctgtct caaggggggg aaaaaagcca tgaaaggctc |
| 12721 | catcccaggc agaggggcta acactagtgc agacgtggtg gtgtggagat gcatcagggg |
| 12781 | agtgtggagg ggagaccgga aggcagggca cgggggacgc agccaaacac ggagggtctt |
| 12841 | tggtgtcaca ctcttagcag gagaatggga tgctaagatc ttgttttgga gagaaggctc |
| 12901 | tagaggtgag gactgcgggc agggacactg gggaggaagc ccggggcagg cggagggctg |
| 12961 | agtgcctcag caggcgccat gagagaggaa ggagcggcgc tgggatcagg ggagtgagtg |
| 13021 | acagggcagg gcaagagtca attgtggtcc tggggaccac cgcggaccca gggatttggg |
| 13081 | gtacagggcc ttagttgatt ctcatctaga caggtgccag ttgctgacac cctgaattct |
| 13141 | ggcaaacatg gagacatcag ggtggaaaca cctggagcca ctgccatcca ggtgggcgcc |
| 13201 | ttgagggcca ggccccttgg ctgctttccc agcagcagca tggagcctgg cacagaacag |
| 13261 | acctggcata gaagtgggag caaggaacac cgcctgtggg tgcaggagca gtgatcagtt |
| 13321 | agcaacaggg gcccccctgc agccagccat ccaggcagca ccagacagtg atccatttct |
| 13381 | ctcggtgaca gttctagaaa aggctccgga gacagggact atcatgtgat catttttacc |
| 13441 | ctagaccgct tgaccgcttt ctgtctgaac tgatccacct gctagacgct gcctcaacag |
| 13501 | tcctcgtttc tattttattt tcttttactt caaagcagaa gggctaatct gctgaggtgg |
| 13561 | agacgagttt tcacccactg gctttttttt ttttcagccg aagtttaagg taaagtcagg |
| 13621 | actgcagttt ccacaatatt tttaccctat cggagagtaa aagagtcacc ctcccattat |
| 13681 | acctcattgt attcccagtg cctggcacag aatgggtact ctacacactt gtggatcact |
| 13741 | gggaccacaa gctcctctgt caccgtaaca ggggaatcta ggacccagca gttgatgact |
| 13801 | gtgtgggtgg cactgtccaa ggctcaaaga caaataccac aggcccttcc tgcctcagtg |
| 13861 | ccagggtctg aaaaatggga accagcgctt tcagtcggtt gcagggcaaa gtcagagttt |
| 13921 | gagtgaggac ctggaagaag ggtttgagct gagccagaga gatgggattt atcatccctg |
| 13981 | ccctgcccct gcctctcaga tgaatccctg acccaagcct aagtttccat agtgtaaacc |
| 14041 | tggatgttag cagggtgggt taaggggaaa tacatgagtg gaggtagaaa tgatatttta |
| 14101 | gtacaatttg tggataataa agggttagag ttccatgggg cctccattta gctgagaaat |
| 14161 | ggggaaaatg gaaggggtaa ccttttccag tgggaggccc agggactctg cagtatccca |
| 14221 | agtctggctc tgtgagtcat tggtgatttt cagagagtat actatgtaag aaacatgagg |
| 14281 | aacagctgct ttcttggaca actctttgaa gtgaatttct ttcttctacc atgccatcaa |
| 14341 | ctgtcattat aaaattgaag atgtgtttct catggggaaa aaaattgtta ttacaagaga |
| 14401 | tatgggcaat tagacaagtc tcaatacatt tcaaaagact gaaatcttac agagtatgtt |
| 14461 | ctctgaccat agtggaattc agctaaaaat caataataag aagattatct ttgaaagttc |
| 14521 | caaatatttg gaaattaatg cctttcttaa cctatggtca aagaaggaag taaaagagaa |
| 14581 | attatgaaac atttttaact gaattataat gaaaatacaa catattaaca tttatcattt |
| 14641 | taagtactta cagcagaaaa gaagaaaagt gtaaaattgt aatctaggtt tctgccttaa |
| 14701 | aaagatacca aaagaaaagc aaattaaacc caaatcttga aaacattatg ttaaataaaa |
| 14761 | gaagccagac acaaaagacc acatactgta taacctcatt tacagtcagg catcgcttag |
| 14821 | tgataggagt acattctgaa aaatgcatca ttaagcgatt gtgtcattgt gtgaacatca |
| 14881 | tagagtgtac ttacacaaag ctagatggta tagccaacta cacacctagg ctacatggta |
| 14941 | tgacctactg ctcctaggcc acaaacgtgt acagaacgta actgtattca atgctgtagg |
| 15001 | caatcatagt acaatggtaa gtatttgtgt atctaaacat agaaaagata cagtaaatac |
| 15061 | aatattataa tcttatggga ccactgttgt atatccaagt ggtcgttgac caaaacggtg |
| 15121 | ttatgtggtg catgaatgta tacgaaatgc tcagaacagg caaatccata cagagaggag |
| 15181 | atttgtggtt gtcaagggct gtggggaggg agaatgggga gtgactgctc acgggtatgg |
| 15241 | agtctccttc cagggtgatg aaaatattct ggaattaggt agtggtgatg ggtgtacaac |
| 15301 | cttgggaata tactaaaaac gactgaactg tgcactttag aaggatgaat gttatggaat |
| 15361 | gtgaattata tttcaataaa taaaaatcaa acacccccca ccaaattaaa cccaaggaca |
| 15421 | gtataaaagg aaggaaatag aaaatagaca acagagaaat ttaacaaagc caaaagttgg |
| 15481 | ttcttcaaaa aaaattaata aaatagataa accctagcaa gacttatcaa aaataaaaag |
| 15541 | caagaaaaca caaattacca atatcaggaa taaaagaggg aatatcatta catttaaagg |
| 15601 | aaaatatggg aatattacca ataacttacc tcaataaatt ctacaactca gatgaaatga |
| 15661 | ataaatcttt tgaaaaacaa cttaccaaaa ctgacagaaa aagaaatgga aaatccaaag |
| 15721 | agcctccata tctattaaag aaattgaatt tgtaataaca atgatgatga tgacaataaa |
| 15781 | cttccttcca caaagaaaac ttcagatcta gatgatttca ctgctgtatt gtagaaaaca |
| 15841 | ttcttttttt ctttttttga gatggagtct cgcactgtca cccaggctgg agtgcagtgg |
| 15901 | cgcgatctca gctcactgca acctctgtct ccccagttca agtgattctc ctgcctcagc |
| 15961 | ctcccgagta gagcagctga gactacacgc aggtgccacc acgcccggct aatttttgta |
| 16021 | tttttagtag agacagggtt tcaccatgtt ggccaggctg gtcttgaact cctgacctca |
| 16081 | agtgatctgc ccatatcggc ctctcaaagt gctgggatta caggtgtaag ccaccacacc |
| 16141 | cggcctgtag aaaacattga aaggaaaacg taataacaat cttacacaaa ctctttcaga |
| 16201 | aaatagagaa ggaagagcac atctcaattc attttttatt atcaaaacct gacaaagacc |
| 16261 | ttacacaaaa agaaaattac agcccaatat ctctcatgaa cacagatgta aaaatcctta |
| 16321 | acaaaatatt agccaactga atagcaatat atgaaaagaa taacacatca tgacaagtga |
| 16381 | gtattatccc aggaatgcaa tgttaattta aaagtagaaa atcaaaatat tttaccatat |
| 16441 | taacacagta aaggaaaaaa ttcatgatta tatcaataga tgcagaaaat gcatttgaca |
| 16501 | aaattcaaca cccattcatg ataataacta tcagcaaact accaataaaa ggagcttcct |
| 16561 | caatctgata aaaatcatcc agctaacatt atacttaatt cagaaatatt gaacattaag |
| 16621 | atcatgagtc aggcaaagtt ttctactctc accacttctg ttcaacactg tgctagaatt |
| 16681 | ctagccagta caaggtggca aggtaaaaac aactcctaca gattggaaag gaagaagtaa |
| 16741 | aactatcttt attcacagaa aacctgattg gttatgtaga aaatcctaag gaatctatta |
| 16801 | aaaagctact acaattaatg aatgaattta gcaagttttc aggatatatg attaatacac |
| 16861 | aagaaaataa attttaccaa agacaggaaa gacttccacc ctaaaaacaa aacaacacaa |
| 16921 | aatgctgaga gaaataaaaa aaaaaaaaaa ccaaacatgt agaaaataga ttatgtttat |
| 16981 | ggactgaaga ttcataattg ttaagatgac aattctccac aaaatgatct atagactgaa |
| 17041 | tgcaatccta ataaaaatca caacaaacta ttttgtagaa attgaaaact actttaaaat |
| 17101 | ttatgtggac atgcaaatga tctagaatag ctaaagcagc cttgaaaatg gagaagagaa |
| 17161 | ttggagggtt cacaccacag ctgacccctg aacaacatgg gtccacttat atgtggattt |
| 17221 | tttttttttt tctttttgag acagagtttc actcttgttg cccaggctgg agtgcaatgg |
| 17281 | agcaatctca gctcactgca acctccacct cccgggttca agcgattctc ctttctcagc |
| 17341 | ctcccgagta gctgggatta caggcatgca ccatcactcc cagctaattt ttgtattttc |
| 17401 | agtagagacg aggtttcacc atgttggtca ggctggtctc gaactcctga cctcaagtga |
| 17461 | tccaccagcc tcggcctcct aaagtgctgg gattacaggc atgagccact tcgcctggcc |
| 17521 | atggtttctc ttccttatct tttttcctaa cattttcttt tctctagctt actttattgt |
| 17581 | aaaagtacag tacataatac atatacaaaa tatgtgttaa ttggctgttt atgttatcat |
| 17641 | taaggcttct ggttaacagt agtctattag tagctaagtt tttggggagt caaaatttat |
| 17701 | atgcagcttt tcaactatat gaggggctgg tacctctaat ccctgtgttg ttcaagggtc |
| 17761 | aactatattt aattataagt ctaactataa agttaaagga attaagaaag tgtggcattg |
| 17821 | gtctaagaat agacatatag accaatggag cagaatagtg tccagaaata aactctgtac |
| 17881 | atataaaatc aattgatttt caatcaaggt gttggatatc tagaactagg tatctatatg |
| 17941 | gagaaaaaat gaacttcaaa ctcctacctc ataccatcca caaaaattaa tgtaagatgg |
| 18001 | attatagaat taaacattaa aaacttataa gcttctagaa gaaaatatag aacatctttt |
| 18061 | tttgtattct tgggttaggc aaaggtttct taaagaaaaa ttttaaaaga ctaaacataa |
| 18121 | gaaataaatt tcactaaaaa cttgcatttc ttaaaaattg gaaactctct ttggctagtc |
| 18181 | acagagtaaa aaaaattgaa aactcctatt cataaaaaaa tcacatttaa gaaggcaaga |
| 18241 | gaaggcaagt cacacacagg aagaaaatat tcacatttat ctgacaaagg acttgtagcc |
| 18301 | agaatatata aagaatccct ataagtcaat aataataaga caaattatga aattttttat |
| 18361 | gtaggcaaaa gacttgaaga gacactccat agaataagat agacgtatgg ccaataaaca |
| 18421 | tgtgaaaatg tgatcaacat cctgagtcat tagagaaatg aagatcaaaa ttacaaagag |
| 18481 | atattatttc acatgctcta aaatagctaa aatgtaaaag tctgacagta atcaaatgtt |
| 18541 | gatgaggatg gaacaactga aatttttttt acattgctga taagagtgtg aaatgatgca |
| 18601 | atccccttag aaaactattt ggcgatttct ttcttttctt ttcttttttt ttttttttgg |
| 18661 | gatggcatct cgttctgtca cccaggctgg agtgcagtgg tgtgatcttg gttcaatgca |
| 18721 | acctttgcct cccgggttca agcgattctc ctgcttcagc ctcccgagta gctgggacta |
| 18781 | caggttcgca ccaccacgcc tggctaattt tttttttgta tttttagtag agacagggtt |
| 18841 | tcaccgtgtt agccatgatg gtctccatct cctgacctca tgatctgcct gcctcagcct |
| 18901 | cccaaagtgc tgggattaca ggcatgagcc attgcgcccg gccagcaatt tcttataaag |
| 18961 | ctaagggtac ctatcctgtg acataaccat tctgttctta ggaatttcct caagagaaat |
| 19021 | aaaattatat tttcagaaaa agactcacat gaatgtttac agcaactttg ctcatatagc |
| 19081 | ccaaccctag aagcacccaa aagtttatca tcaacagaga aatggaccaa gaagttgtgg |
| 19141 | tataggcatg caacgggcta ttactctaca ataaaaatca atacattact tatatatgca |
| 19201 | acaacatgga tgaatcacaa aaacattacc ttgaccaaaa gaagccagac acacaaaaag |
| 19261 | tacattctgt aggatacctt ttatatgaag tttaaaacag gcaaactgat ctttggtgct |
| 19321 | agaaatcaga gcactggtta ctaagggact agaaatcgac tggaaaggga catgagagaa |
| 19381 | catgtggaac aaatggaaat gttctatgtc ttgattgtgt ggcggtatac agggaaatgc |
| 19441 | atttattgaa actcattgaa ttttacactt aagatccatg aatgtaagtt tcacctagtt |
| 19501 | ttaaaaaaag aagagagact cacatctaaa atacctaaaa cttgaaaaat attatataag |
| 19561 | atgaggaaaa aaattatttc tgattccatg ctactaaaaa taagctacaa atgtgattca |
| 19621 | taatagaaga agccattttt atatttagca cataacttaa aggtctaaag gacaagcaag |
| 19681 | ctgggcgcag tggcccacgc ctataatcct agcattttgg gaggccaagg caggaggatc |
| 19741 | acttaaagtc aggaatttga gaacagcttg gccacatgct ggtgaaaccc tgtctctcta |
| 19801 | aaaatacaaa aaaagttagc caggcatggt ggcacacgcc tgtaattcca gctacttagg |
| 19861 | aggctaatgc aggagagtca cttgaaccca ggaggtggag gttgcagtga gcggagatta |
| 19921 | cgccactgca ctccagcctg ggtgacagat caagactccg tctcaaaaaa ataaattcaa |
| 19981 | aacaaaatag aatgaaggag aaacaagact atttagtaga ctgagaatag ctgtttctta |
| 20041 | ttgagggata aactgcacca aatgctattg taagcattat tatttccttc attttaaggg |
| 20101 | tgaggaaaca agggcaaaga gaagttaatt agcttcatcc agggcacgca aactaggaag |
| 20161 | aggcagctgg gatttgaact cagggactct ggcccaagag ccttaacctc tatcccatgc |
| 20221 | cccctcgtgc ttgtaaagga cttgatgctt tacttagact ttcccacaac agtttcttgt |
| 20281 | catcctcgtg atggtcccag gacacaggct ggtccaggat aatgaccccc ggctaacaga |
| 20341 | tggtgagtct gagaccccaa ggggttggat ggccagctcc caggcctcca gaccatgggc |
| 20401 | cgttgtgccg ggccgcacac ctgagtgggc ctggcttttt cctgcacgca ggggtgcttg |
| 20461 | ggccctgctt cctggtgctc catgggagta tctggccttc aaaaggaaca gatcgatctg |
| 20521 | aagtggggag agagaaaata cagcaggacc atcactcaga gagtcgctgt agaggaagag |
| 20581 | agggagagga gacgccagga catgggagct ttgtgggagg gaatgggact atttgctgag |
| 20641 | ctttgtacag cccagcccag gcagtgcata caacaaggct ttgctgattt taaataacac |
| 20701 | aaggaaatag cagccccagg tctgagtcac cagcagagtt ttactctatt tctcagctca |
| 20761 | cacagggaca atagcaatgg caggcactgc tgggagatag gctagtgggc tgggccctgg |
| 20821 | caaaatcacg acgtccttga aaacagtccc aggaccttct ccttcccctt cggatggccc |
| 20881 | accacctcac cctctgccct ttcgggaccc cagcccctct ccagtcttgc ttccacaggc |
| 20941 | tgctccctga gggcagacca ttcttgcttt tcccttcacc atcgggactc ccctgtcctg |
| 21001 | tgtccagctc cttggaggct ctactctctg ctctacgctt tcaaacattt ccttacactg |
| 21061 | ttctctctgc ctggatttcc ctccttctcc ttgcttccca gtctctctga atatccagat |
| 21121 | ttcttctggt cttctagcgt caatgcgtgt gcctctgcaa tgcctcctta atccactaca |
| 21181 | ttgagagtca ttgctccttt tcctgtgttc tcactttatt tacacttttt gcatgccaat |
| 21241 | gacaaaattc tgccttgtat catcggcaga attttccatg tctgatatat ctccctctgg |
| 21301 | attgtaatac gctcttaaag gcatcgttca gattcgaatg atcttagtac aactcccctc |
| 21361 | tgtgaaggcc aaatggcaca caagccagga gctagaaagc cctggttgcc tgtggaaatg |
| 21421 | aatgaacaca catgggctgg cagccgtgtc cctgcacacc tgccttttct cagaggtgtg |
| 21481 | gccctagcag ggtaagctgt ctggaagagg gagctatttt gtgcagatgg aacattttgt |
| 21541 | ccccttaaat catgtcccat gaaaggccag catggcatcc tgcttctgaa acaagcactg |
| 21601 | agctcgtctc catttggaga agatctgtcg tgtgggtatt tttaaatggt gaaaggagaa |
| 21661 | atgggtatat ctgataccat ggcacttgtt acactttaag cacagactct gttattcata |
| 21721 | ctccatctat tgactcaggg aggagtttat ttcaaaccaa acagctcagc tatcccagga |
| 21781 | gcagctgcct cagaaggaag gaggatgctg ttctatttcc cccatcgaac ctccttctca |
| 21841 | ctcgatccac aacagccttt ccatgcaagg aagaggctat tttctttcaa gctttattct |
| 21901 | ccattttccc tgtggcaaga aaggtgattg gatattggga tttgtaatgg ttttatctga |
| 21961 | aagccattta aaatgaagct ggaatgtcaa caagactgac gctgaatgga gtagatgtga |
| 22021 | agtggaaatg aacttgaaac aagagatggg gttagtcatc cccctgatca cttggaacag |
| 22081 | atgatggagg tcccctgcct ctgctccagt gaacagaaca gaaccacttg aatactgcca |
| 22141 | gccactgttt gtacataccc tcctgaaacg gctcttccaa aatgctgaag gacattttgt |
| 22201 | tttcttaata atttggcctt tccactgttt ctgtatgatt gatgtgtttc agcaaacctt |
| 22261 | ttttaaaaaa gaatatctac aggtaaattt agctcatcat gtttgagaat attcaccatc |
| 22321 | tctaaatccc cagggcctaa atcaatgagg ttttagcaca ttttgaaaca gaaccctgat |
| 22381 | gtataaattc tgcaatggct gatttcacgt attttacttc ctctctaaca aagctatgga |
| 22441 | aaaatgcaca agcaggggag ggtttgatcc tcggtgtttc ctgcagatgt gaatctgaca |
| 22501 | ccaccttgga gcagggcccc tccccacctt ctcccaatcc tccgctgggt tgctgcattt |
| 22561 | ttcactccaa gagaccccgg aggacagcta tatccgaggc ttattaaaat cttctaggat |
| 22621 | gcaaaggcct ttggttattc agagcagcag gtctttaggg gaagccccca tacacaaaca |
| 22681 | tgctcagctg attctcaatt tatattgtgt gttacctgcg gggaaaatta aaagctcaaa |
| 22741 | cctcaggcac tttccctctc ctcccttgat tgcccggggg cttgttgttg agatatttct |
| 22801 | tgttgatttt tttgtttttt atgtctctgg ctggtgctaa acattctttc tttcccatca |
| 22861 | tgtttacttc acatctcttt gccgtataga tagcttctgc tttatggttt tttcctcctt |
| 22921 | atctcagatg cagccagccc ttccccacct gcccccgccc caaaacacac acacgaacac |
| 22981 | acaccctttc agactgcatc cacactctct gtttcagtgg atctgacatc aacgaccccg |
| 23041 | ccacccccgg ccctcgtcag aggggagctc tgcattgaac taggactagc tcctgggtca |
| 23101 | taaaagttgc taggaccctg gagagctctt actaaacaga tgaggccacc actgacagag |
| 23161 | gagagaggtc agttcccgga gggaacgagc ctgctttgct gagccccctt ccttgcccag |
| 23221 | cccccagacc catcacaggg aaggttcaga ggagctctga cccctccagt acctccttgg |
| 23281 | cctgacgccg ctctagcccg ggctcccagg ttttctgtag ggagaggagc acagtccgct |
| 23341 | ccacccatgc catgggcttc atcagtgaca acccaaactc acccccaaat aggaatggct |
| 23401 | tcaacctgga gacagccctt caatgaaatg gtttatgctg gtggagattt aaaggtattc |
| 23461 | gtctttcggc tgggcgcggt gactcacacc tataatccca gcactttggg aggccgaggt |
| 23521 | gggagatcac ttgaggccag gaattcgaga ccagcctgac caacatgcga aaccccatct |
| 23581 | ctactaaaag taaaaaaatt agctaggcac agtggcgcat gcctgtaatc ccaactactt |
| 23641 | gggaggctga ggcacgagaa tcatttgaac cccggaggca gagtttgcag tgagccaaga |
| 23701 | ttacactact gcactccagc ctgggtgaca gagcaagact ctgtctcaag acaaacaaaa |
| 23761 | agggtgtttg tctttcaatt gcaaaaatca agcatggtca ctgaaaaaaa attcaaattc |
| 23821 | acatttcatt ttgttttgtt ttgttttgtt tgtctttcag ccaggtgcag tggctcacat |
| 23881 | caaagtatgc tgctttcaat atcttctcac tctccctccc cagggataac cactttcttt |
| 23941 | tttattttat ttatttaact tttttgagat ggagtttcac tagttgccca ggctggagtg |
| 24001 | caatggcgca atctcggccc accacaacct ccgcctcctg ggttcaagcg attctcctgc |
| 24061 | ctcggcctct tgagtagctg ggattacagg catgtgccac cacacccagc tatttttgta |
| 24121 | tttttagtag agacggggtt tctccatgtt ggtcaagcag gtctcgaact cccgacctca |
| 24181 | ggtgatccgc ccgcctcggc ctcccaaatt gctgggatta caggcgtgag ccactgcgcc |
| 24241 | caggggggta accactttca gtggtttgag atgtttgtct cttcatagaa gaaatatatg |
| 24301 | catatagttt gggttttggt atttttttta attaatggag ttctaatata catgctgcct |
| 24361 | tttttccctt accatggaat tgacacagga taataaatta attctaagca acaaattttt |
| 24421 | ctttaaccca tcctgtctct aggtttctcc tcacagaaat tacaacccct tctcttgtgg |
| 24481 | ttccatccca tccctaaaac agtatttatc ctcccttctc tgtaccatta ctgtttcttg |
| 24541 | tgcgaaattg taccttggag tatgtactga atggtgttgt actggaggcc tggcgcggtg |
| 24601 | actcatgcct gtaatcccag cactttggga gaccaaggtg ggcgggtcat ttgaggtcaa |
| 24661 | gagttccaga cgagcctggc caacatggtg aaaccccgtc tctactaaaa atacaaaaat |
| 24721 | tagctgggca tggtggcggc gcctgtaatc ccagttactc aggaggctga ggcaggagaa |
| 24781 | tcgcttgaac ccaggaggcg gaggttgcag tgagccgaga tcgtgccact gcaatccagc |
| 24841 | ctgggaacag agcaagactg catctcaaaa aaaaaaaaaa aatggtgttt cccttgcact |
| 24901 | ggtgccctgt tggctcctgg cctcccttct agaccatcag ctacctgtgg gtgggagcct |
| 24961 | gtgcctttgt cattggtcca tcctctatcc cgaggccccg ggacatggcc tagaacatca |
| 25021 | gagactccag cggctgtttc tcaaagtgaa ctgaactagc tggtgtttcc cacacgatgg |
| 25081 | agccccctcg acctgccctc gattctctcc tgacttattt ctccccacat cttgggccag |
| 25141 | ttccagctgg actagcaaag agggcagctt tacctctgtg ctcccaacat gccctagtgg |
| 25201 | gtggcgtatg gatgggggag cctgggacaa tgtgtgtcca tctcacacac tctctacttc |
| 25261 | ccatgccagt ggggaaagtg ggggcaggaa tagagcccgg gagggaactg ccagagactg |
| 25321 | tcttctctgc taaggctgac aggaagacgg acaaccatga cagtggtgga aggggaaggc |
| 25381 | ccttggccca cggggaatgg ctgtctggaa gctgacttgt tgttgggggc ctggtttggg |
| 25441 | caggaacccc tggggtggct gcagaaggag gctgggcttc acagacccct gtcggcaggc |
| 25501 | aggggctacg ccccctatac taggcttctg ctcagggcag cagggcccgg agccaccagc |
| 25561 | agagctgagg aggctgcaga ggaggctgag agatccatgg aggaagaaca tggtggaaaa |
| 25621 | gagtgcctga ggtccgcttc cacggccact ctgaccagcc tgggccttct gtgccttagc |
| 25681 | attcccgttc ctaacatgga actcatggcc tttgacccgc ttccctcacg aggctgctga |
| 25741 | gagggtcaga gaagaagccg catggggact ggcctcaccc tgggagaggt gagcaagcca |
| 25801 | cgaggtccca tgttgcatga aaagtcaaat gctgttagga cgactcccag gatgaggagg |
| 25861 | tctgacccca tgctcatcgt ctggacagct ctgaccctag aatttatttc ctctttcccc |
| 25921 | acagggcaat ccttctactc tcgggtcctg gagaactgtg aggatgtggc tgactttgat |
| 25981 | gaggtaggag gcccaagact cccccctccc tcagccctga gcccagaggg gaacagccac |
| 26041 | ccttcgctgc ctcttgcggg cacacactgg ccccaaatcc caggcactgg cctgctctga |
| 26101 | gcccctcacc tctcagctgc ttcctgaagg aggtggtaag gagggtgtgc aggaactccc |
| 26161 | tcccccatgt tccccccaga aacctctgca caggtgaggc ccgtgtgcaa atgacacggg |
| 26221 | gaacagcagc ctcgcccacc tcgacggccc tgctgggtct gggcctgtta ttccctgtgg |
| 26281 | agaaagagcc cacaggtgct gtagacctga atgtgagcca ccagctgagg gcccaacgag |
| 26341 | gcaggcgagt gttggggacg tcgcaaaggc cagtctgtgt cagtccccag gggttgaaga |
| 26401 | gcagggcagc cccgggcact gaggtaggag ccccagtcac cagggaaaca ttgtcaagca |
| 26461 | ccccacctga gtgagggcgc catctccaga ccttgagggg aagcataggg aacagaagac |
| 26521 | agtctggatt ttcagaaaca gctctggggg ttccatgaga aaagacgtga gctgctggtc |
| 26581 | tattgacaac gatttttctc aatcctcact ccagctgggc tgttgtcaca attttatcag |
| 26641 | caaaggagca ggcgacacca tcccacaggg gtgcccagga gcagtgagag gacttacaac |
| 26701 | gcttccctct ccctgccctt cccctcggta cctggccggg ctgggagccc tatcctcaca |
| 26761 | cacacacacc agctcgagca gcctgcggcc tcctccgcct cctcacattc tttcgtctat |
| 26821 | gtgtttgctg cttccagaat ctatattccc ctctggctgc tgtaggttct ccagggatga |
| 26881 | ctttggagtg agggccagca gtcatccgtc accatcttag cacaaactgg aagccatgta |
| 26941 | gtgaccctca aatagtgtac cccctctctc ctggggttgg ctctcccgct tggctctgtg |
| 27001 | ggggctggga agggcctcat agcagactgt gcctcttgag gttggctgct ggcgttcggc |
| 27061 | tgagcagcca ccaggctgac acttggcacc agctgtctcc ccctcctcca gaaaccaggc |
| 27121 | cacacgatgt ggctgtgtcc atcgacccag agctggagac acagggagca aaacctcgct |
| 27181 | cctcactcag gcttaggcac agccatcaac catcgcggcc cagcaccaca ccctccccaa |
| 27241 | ccacttcagg gcgcagttgc cacgacctcc ctggtacgtc cccactgcta tgcaggacag |
| 27301 | gtgaatcact catttaaagt gaatcactag agctaaggaa gtttctgtcc ttaccttctc |
| 27361 | cccctcctgg ctgtacaatg acctcagttt ctaaacctgc caccatcttt gtcaaatggg |
| 27421 | gttggtgatg ggtgtgaagg tctctgtgaa atgatgaaca aaatgaaggg ttatggtgag |
| 27481 | agtggaatca tggctgccca ctgagacagc tgtcatagat gcttcaggct tgccccatta |
| 27541 | cacacccggc ctgctccctc ccctccccat ttcctcagtc agggcactgg gctggctggg |
| 27601 | agaagatgcc agcagcctgg cccagtttcc catagtccta cctccctggg cttctgccca |
| 27661 | ctaatccacc aggcacagca tctgactccc tcctcccaag actcccaggg gatatgtcag |
| 27721 | cccaggcgtt cagccagggg gcaacctgat gccccttcca agggcactgc cacccttttc |
| 27781 | catgcccagc gccttggaca cctgagccac aggtatgcac gacattcaaa gttaatcacc |
| 27841 | atgtcgttcc tcttcccaac tccgcccagc ctgcctcagg gggagagtct gtctctgctg |
| 27901 | gccctgaaca aggggccaca ttccgagcat ctagtgcatg ccaggccggg cttgagatat |
| 27961 | cccagaggca ggaacctggg ctcaacaccc catgatgatt tgtatgggag cttagggatg |
| 28021 | gggtagccag agtcagtgtg gagagactgg ggcagggcag aagcagttat ggaagaagca |
| 28081 | catgactagg gtcagagagc agggcacgcg atggggagaa gaggctcctg cccggggaaa |
| 28141 | gtgctgcccg cccactgggg agacagggaa gtttctggtt gttgcaatga ggagcctact |
| 28201 | ggcatttagt ggggtgggga gcaaggatgc agaatgactt gcaatgctca ggccatcccg |
| 28261 | cacttcccac ctgggagccc cgtgggaaga tggtggcagc cccagcagca gcactgagct |
| 28321 | gcttcccgag ccatgcccct tccactcaat ctccgaaatc atccacagac gaaattaggc |
| 28381 | tcagatggta actacccttc cccaggtgac agagctggaa gcggccaagc cagggttctg |
| 28441 | aattcatgca tagattagac ctaagccctc tctggataca gtgatagggc ctgactccca |
| 28501 | ccacccctgc aaggcccgtg ttggagtctc tccacacccc ctgcctccat ttctccctgg |
| 28561 | gcaaagtggc ctcagccctg gccaccaatg gtcagcagtg cacatcccac aaagcaggaa |
| 28621 | ggccaccctc ccattccctg ttcaggaacg gcccggaggc ctgggtcagc ccagtcccaa |
| 28681 | aggtagcctg gagccatacc ctggctttaa cctttctccc tagacccagt ggacaagttg |
| 28741 | taaacttccc tggcttcccc ctgaaagaat ctgcctgcca gtggttccag aatatcctgg |
| 28801 | gaaagagccc tttcaaatcc ccctgaacct tcttgacttt ccagaaagcc tcttctctcc |
| 28861 | gtcttactct gcccagggcc tgccggagcc agcttagtga gacacactgg cctgggagtc |
| 28921 | atgagaccta caccacagtc cttgctgagg aacctctttg ggctttggat tccccttctg |
| 28981 | taaaagagag atatgctccc tgctctatct gccccacggg actgtcacaa ggctaaaaaa |
| 29041 | agatgaaatg agagacaaga aagcaaatgt ctctcacagc tctactaggt aaggggttcc |
| 29101 | aagcaaggga ggggcccctc ctctgctcag gcatgggtga gaggggctgc agcagctggg |
| 29161 | cttaggaacc tgagtgggga ggaggatgcc atggaagaaa gacaatgctc tctacaacaa |
| 29221 | tgggtggaaa cagagcctgg acatggtgtc tcctttccca gaggctggaa cctgagcccc |
| 29281 | tacatttaac ctacaactct ggaaaagcta gggagagaca gagaaagacc cagcctttgt |
| 29341 | gacctgggga aacaggagta gccaccttta gccagtttgg tttggctcta acacaggccc |
| 29401 | tgtgtggagc aggccccagg ggagaggcca gagtaacaat agtacttgtg ccaatgataa |
| 29461 | gaagaactgc tgctatttac tgagcactta gtgtgtggca agatgggctg agagcttgac |
| 29521 | acaagcatta gactcactag aacttgctgg attggtagca tcctaggtga taaggagcca |
| 29581 | caacaggatt taaacccgag tctatctgat tccaggacct atggtcattt gtaaagtaga |
| 29641 | caagactcag ccctgccctc aaggggctca gtctagcggg cagatggacc cactacaaac |
| 29701 | aaggagggac cctgggcctt ctgagccaag aggaagaaga ggtctccagc ctgggggatg |
| 29761 | acagggcagg ggagtttggc cttatctgcc cctttctgct tgtaccagga agtcatggct |
| 29821 | gttacctcct ggaggccaga aacgcacctg tcttgttcgt cattggcctg gcacccagca |
| 29881 | tggagttgag aaaatgttgc cgagtgaaag aatgaaaaaa acagtgacgt tcactaggac |
| 29941 | tggcacctgt caccgcaggc ctgtgcccag accgagtggg ctcagcaggg ctagtggtac |
| 30001 | atcaagccct ttgctgtggc ctggatagaa aggaagcagg caaggcacag atgagatcac |
| 30061 | ctaaagggac tttagatcac cttgatctct aaggttagag atcatctagt ttaaccctct |
| 30121 | tgtcttgcag ttgaagaaac aggtcttaga gtggggaaga gaattgccca agaagaccag |
| 30181 | ctgagatctg agatcctaca tttaacttac aactctaaag aagctgggga aagagacaga |
| 30241 | gacaaagaca gaaacagaga gagagagaca gagacacaga gacccagcct ttgtgccctg |
| 30301 | ggaaagtgaa atagccacac tcgggatact gagcccaggc ctttaagctt gcaaatccgt |
| 30361 | ctctttccat tgtactctaa agcagcagtc cccaaccttt ttggcaccag gggctggttt |
| 30421 | cgtggaagac agtttttcca cagactggga tggtggtggc tgggggttat ttgggatgaa |
| 30481 | actgttcctc ctcagaacat caggcattag attctcttaa ggagtgcaca gcctagatct |
| 30541 | cttgcatatg ttgttcacag tagggttcac actcctatga gaatctaatg ctgtcactga |
| 30601 | tctgacatta gatggagctt aggcgggaat gctcgctcac ccgcggctca cctcttgctg |
| 30661 | tgtggctctg ctcctaacag gccatggact ggtactggtc tgtggcccgg gatatgggga |
| 30721 | cccctgctct aaagctttgt agtgcatctc aatattttcc cccaaaccta tagccctgtc |
| 30781 | cctttaagga tgctaccttg ctgatttagg atggttccag gcccactcag cggccaagct |
| 30841 | ggacataatc cagatcattc tccaaggtac atcagggaaa ccttttttgc agagtcctag |
| 30901 | atcactgggg ctgataggat gcagaaccct gtgccccggc cagcaccagc caggtcaggg |
| 30961 | ctaagcctag accaggaaac tgggtcagga ctcagcactc cccgggctcc tgagatcact |
| 31021 | ccctccaaca gaggccctct ttccctccct atctgtacta cctaccacct tgcttctgtc |
| 31081 | aacagtccta tcccaggctg ctattccagg tctgtgtctg tctccagaac tatccaggac |
| 31141 | catccccagg actgagaaac aaccctgcat ccctgcgttc tgcacagcgt catgcacaga |
| 31201 | gcctggcaca cagcaagtgc tcagcaaatg ttcgccccca gcatggggct tgctggctcg |
| 31261 | tgacatcttg agtgactgtg tttggcttaa ctgctacttg agtcccccag cagtaccagg |
| 31321 | cttcaggaga ggattcagtg tagtggatta agccctgagg actgaactcc agactaagca |
| 31381 | tcctgagact tgagtttgaa tcctgcctct gccaccaacc agccttgtgc ccgcgggcaa |
| 31441 | ctctcgcctc tccactttcc cacttgtaaa aggagcacat ctttgccatg ttgctgtgag |
| 31501 | cttggttatg ggtaaatgtg ctctggaagt actaagtcct acccagatga aattcttcct |
| 31561 | tcagaagccc ttttccttca gtcttttttt tttttttttt ttttttgagg tggagtctta |
| 31621 | ctctgtcacc caggctggag tgcaatggtg ccatctgggc tcactgcaac ctctgcctcc |
| 31681 | caggttcaag cgattctcct gcctcagcct cccaagtagc tgagattaca ggcatgcacc |
| 31741 | accacacctg gctaattttt gtattttagt agagaccgtg tttcaccatg ttggccaggc |
| 31801 | tggtatcaaa ctcctaacct caaatcatct gcccactttg gcctcccaaa atgctgtgtt |
| 31861 | tgcaggtatg agccactgcc cccagccccc tttccttcaa tctaatagct cacaagttca |
| 31921 | ataccatagt caggagagga gtgtagctcc caaaccctca aaatcctcat ctcgtcttgc |
| 31981 | tgtgttctca gcatatcacc ccaaatggag gaaaggacat aaccacaaaa atgaggagac |
| 32041 | aatgaaaaat gaaattaggg aagagaagat gcagtgaaag agacacgtga aatccacagg |
| 32101 | ccgaggcaga agaggaaagt gatggcggcg aaggggaggg aggtggcatg acaaggagac |
| 32161 | aaggcagttg cagacctgca cagagttccc actgggaaga tctaaactaa acacattctg |
| 32221 | ccgtgctgtg tcacttacga ctgctgtcac ctgtgagatc ccatttgatt ctctcaatag |
| 32281 | ccctctaagg tagatattgt cacccccctc ttttcacaga tggggaagct gaggcacagc |
| 32341 | tagtgagtgg cagagccagg gctagaactc aagtctcctg actcaaactc agagtttctg |
| 32401 | caacaccacg gagactccca gtctgcgtga gatacaagcc tcggtgttgt cagaagaaca |
| 32461 | aaaaagaagc aaagggtggg catgtccttt ggagctctgt gaggtgccca gagctcacag |
| 32521 | aggctgcctc agcaaaggaa ggtgtcagca atgggagatg gggctggaaa tgctgtctgg |
| 32581 | gcacagcaag ggggcctctg ggtgctagga aggggaatcc ccacctcttt cagggagctg |
| 32641 | tagaggagcc actgaaaagt tttaacaaag gagagataaa aagagaggaa aattcaaccc |
| 32701 | caaaagtctc cttcttacag cccatgcctc tacttccagg gaggccaaat gggagtaacc |
| 32761 | cccagtccca gaccctgagt cacttccctc cagccaagag gcccgtcacc ctccgaggcc |
| 32821 | ccacactgat gccaggaccc cacctagagg tgccggcttg atcggtctcc aggctgggcc |
| 32881 | ttgggatttt taaaagcttc tggaatggat tgcagttcca gccaagatga agaacccctg |
| 32941 | agaaagacag agggagacag agacaaaggg agagatagag acagagagag aaactgatga |
| 33001 | aagaaaacag atgtggtgag gagaagaaag cagacgaatg atgcgggagg gaaggaaaag |
| 33061 | aaatgagggg aaagcctcgt gtgggtggaa aatgcgggag ggagacaggg gaggaaagaa |
| 33121 | aggaaactgt aaggtgggag caagtcagag accgaagaag ctgctgtgac tcagtgagga |
| 33181 | ccctgtcttc tccctcaccc ggaaagcctc caagaagaac attccccgct gacataggca |
| 33241 | tggactgccc agacccggct gacgaatttc tgaaagggca cctgagccac acacctacac |
| 33301 | acgcttcccc tgattattct catctgcagt caggggcccc ggaagccccc tgtctcctgt |
| 33361 | gccctccctg tcccccgact ctcactcaac aaactggccc tcgccccgca gccaggcagc |
| 33421 | ctccctcccc aggtccctta gcctcagcct cccgccaccg ttcaacaccc cacctccctt |
| 33481 | tcacagggcc atctccaaag tcatgtcctt tttctcctga tctttctctc cttccaatgc |
| 33541 | caaaatccca cccacagacc ctcaggggga ccaatcaccc ctcccctctt catccccagt |
| 33601 | aaaccaaatc tcggtggctc ctgcggcccc ccgactccca cacagcgccc tggctgatga |
| 33661 | gctgggcctt cctgggctgc tttatttccc tgcagcccca taaaagcagc ttcattaaga |
| 33721 | aaaccacaca ctctgctgag ctgccagagg catccagaag ctggagccgc agccgctgaa |
| 33781 | tgaggcctgt gggccgggct gcctccactt cctgggactc tcagcgcacc cctccgccct |
| 33841 | gtgcccacct cgcctctgaa gccctcccta ggccctgctc agaaccctcc ccaccccacc |
| 33901 | ccgcagaccc ctctcccgct cctctgcttc ttcctccctc tcatgccaag gccaggtcct |
| 33961 | cccttgccag tcctgaaggg agatgtcaca aaggccgttt tcctgagaca ggccctgccc |
| 34021 | acactgggca gtgacccgcg gtgctggccc agtgtgccca agccctccaa gtccccaggc |
| 34081 | tgggagaagg aatctcagct ttgggaatgg caaacgtgca gggtgggctc acagaagcct |
| 34141 | cctcccgagg gcctgctcag ccagcaacag agtgctccaa gtcatgcatc tgctctgagg |
| 34201 | ccgagttcct tgtttataga gcacagaagc cattacccac ctctccggcc attggaaaga |
| 34261 | ctaaatgaat gaatatgcac gtagagcatt cagacctctg cataagaaaa agttccccag |
| 34321 | aaatggcagc gattattact attattattt attactggac tattgagcag tctgaatttg |
| 34381 | gtagggtata aaaatggaac taaaaaataa aaagtcactg ccttctacca gatctgtcaa |
| 34441 | aatcctccct gacacggtct ctccacagcg tcttgagcag acctcagcgt tgtctggtcc |
| 34501 | tgtccagaag ggcttcctac tccagtctgg catcctgttc acccctggcc atcctccttc |
| 34561 | cctcatgctt gtggctcagg atgtggagtg ggacagggac ccctgctgag gccctccaca |
| 34621 | tcctcccctg gtgggcagcc catggaaaaa ctgtcctcat atcactctac cccacttggc |
| 34681 | caatggagcg caaatagatg acttgccaca gccaacagtg gcaggctggg ctccttgccc |
| 34741 | tgctgtactc tatgggtctc ctgccagtcc actgccctgc agatgccact ggggatttgc |
| 34801 | atgccatgct gcccctcttg caggtatcta atcaccatga gctgtttccc caagaagtac |
| 34861 | cacaaaaaca ctacgcctag gacagaattc ttcccacctt tcccttgccc agtcttcttt |
| 34921 | cccatcccag gaaggtgggc tgggtgatgg ttggaggtaa cagggccttt gaggaagccc |
| 34981 | taggaagatg tctggctctg cccattggca gctttttaca gaatggggca ctgggccact |
| 35041 | ttagcctctg ttttgagcca ttgttgccac ttctagggca ccaggaaaaa tcccaatcaa |
| 35101 | attcctactc atcattaaca ttgagttagg gaggaagcaa cgaagaagtg gtaagggtga |
| 35161 | atctagaaac aatgagttgg ggacaggctg tgggcctgac cccaataaca ctaatcagct |
| 35221 | caaacctgtc cccagtgtta ccactggcag agtgaggagt ggtttgaact caacacagga |
| 35281 | gtctggggca gagcctgtgc cagtcagggt gattctgatg agagagagct ggaagctcac |
| 35341 | agctccattc agcacccacc aaaaaccagg ctcacaccca cattccttgc tgtctccctg |
| 35401 | ggacccgacc accaccctgg ttgcaccacc ctctcccaac ccccctcagg agctgggagg |
| 35461 | ccatggaagc agcgctgtgt agggagtcaa aagtcctggt aggtccaggc tcttcaccta |
| 35521 | ccacatgacc ttgggcaggt cctccaacat ctctgagcac catctttgaa agaggagtgg |
| 35581 | gcaacaatgc cggctctaca aggtggttgt tggaactaaa tgagattcca ggtgtcaatg |
| 35641 | ctttgtccct ggggagcact gggcccaagt gaggggccac tctgactgtt ggagatcccc |
| 35701 | acaagagatt cccctcccac accccacggc tcctacctgt tattgccacc gaagccgcag |
| 35761 | ttgtccctct ctccccagac atttcggtgg ccggtggcca gcagcatcga cagaaatgag |
| 35821 | atgctggaga ttcaggtttt caactacagc aaagtcttca gcaacaagta agtgtgggat |
| 35881 | ggggcaggag gggctgggcc tggacgggct gggagcctgc tcttctcctt gggctaccct |
| 35941 | ctgtttgggg cttaaaaagg ggacctacac tcccaacctg ccctggattg atcttcaaag |
| 36001 | accgaagaaa tcttgggctg gtctttgatc cccattttga tccctcttct tgtcttcaga |
| 36061 | atcccccagt tctcccagct gctgtcagcc aagaacactc caccctccta tcacctctgc |
| 36121 | tgtctccaac cccatcaaag cttcctgtct taaagactta aagctttaag tctccctcaa |
| 36181 | tgccacgctg gggtacagtt acccactgga gaggcgaaag gtggaggggc tgatgatctc |
| 36241 | ctccaagaga cactctgtag ccctgagggc aggcttgaca gtttggggtg gtggtttagg |
| 36301 | agtcaggcaa gcctgatctc agacactcag gtcagtcatc tactgtgtga ccctggaaag |
| 36361 | gttacataac ctctctgatt tcaatgtgct catgtgtgaa aaggacaagt atttacctca |
| 36421 | aagggcagct gtgtgaatta aatgagatag atattcactc actcattcaa caaatgttta |
| 36481 | ctaagcgtcg attgtgtatc aggtactgaa ttaggtgatg cagagggcgg ggtgcctacc |
| 36541 | ttcatggagc ttgcactcca caagtgaagt acacctgcca tacgagcgtt atcactataa |
| 36601 | ggtgttcaat aaaagtagtt gtcatccttc tctctgggcc atgagaagaa ttcagaagga |
| 36661 | tttgaacacg ctggaacagt ggctaagtaa tatcttgggt taaaaaatca cagccaggtg |
| 36721 | tggcacacac ctgtagtcct aggtccttgg gcagataagg cgggaggatc atttgatccc |
| 36781 | agtaatttga ggctagcctg ggcaacatag tgagatccca tctctttaaa aaaaaatcgg |
| 36841 | ttgctcttat acaagctgga ggtcatggct taactgtaac atgtccaaga aggtatggag |
| 36901 | gtgttagctc actgaggact cagaatgact caacagtggg aggagactac aaacaaaaga |
| 36961 | aaagtaggcc tcatcctaaa tcatactaat agaattaaag cactcctgat ctgagcagga |
| 37021 | gagacaagga gtccactgct ctgatactag tggcagagaa ttcctgtagg caccacccta |
| 37081 | tagtttccag gaggaaaggg actttaagtg ttaccttata acaaatggat gagagcctga |
| 37141 | aagggaaaat catagaggac atattcatcc attcatccat ccattgatcc attcatccat |
| 37201 | ccatccatct atcattcctg ttattcatcc atctgcctat ccatttgtct gtccatcaat |
| 37261 | tgatttatcc acccttcctt tctttcatcc atccttccat cctttcatcc atccattcat |
| 37321 | caatccatcc ctccatccat ttatcaattc atttatccac ccatttatcc atctatccat |
| 37381 | tcatccgtcc atcctttcat tcatcctatc catccactca tccatccatc catccatcca |
| 37441 | ttcacccatc cttcctttca tccattcatt catcaaccca atccttccat ctttccatcc |
| 37501 | attcatccac cacttaccct ttcatccttt ccattcatcc ttccatccac tcatccatcc |
| 37561 | attcatccat ccttccacct tttttctttt cctttttttt tttttttttt ttgagacaga |
| 37621 | gtcttgctct gtggcccaca gtggagtgca gtggcctgat ctcggctcac tgcaacctcc |
| 37681 | gcctctctgg ttcaagcaat tatcctgtat cagcctcctg agtagctggg attagaggtg |
| 37741 | ctcaccaccg tgcccagcta atttttgtat ttttagtaga gatggggttt caccatgttg |
| 37801 | gccaggttgg tatcgaactc ctgacctcaa gtgatctacc ctcctcggcc tcccaaagtg |
| 37861 | ctgggattac aggcgtgagc caccactttt ggcccatcct tctatctttc cattcactga |
| 37921 | tccatctatc gatccatcca ttctttcatt ctttcatcca tccttccatc catcatcctt |
| 37981 | ccttccatcc attcatccat cctttcattc ttccatccat tcatccatcc atccatcttt |
| 38041 | tcacccaact ttgcagtcac tcatcataca tatagtgaac aaatgctttc tgccagtcag |
| 38101 | acactatgct agtcactgga gatactgaga tgaggtattg aggcacagtc cctgccccta |
| 38161 | ccctcccaga aggctttttg ccacaggaag aggaaagaaa ggtgtgtacc tagaggcagg |
| 38221 | atgaggaaca ggctgctgga agacagcaga caactgaaag atagccagta ttttccagcc |
| 38281 | aatgtgctgt cttaggaggt agtgagctcc ttgccactgg gagagggtga gcaaagagtg |
| 38341 | gaccacattg tagaggggat ttctgagtca ggaaaggccc caccatatgg gctccaagga |
| 38401 | gtctctgatt ctgtgggttt tattaggagc tttcaaaatc tcagctgagt atggggcaag |
| 38461 | ggcatttcag aaatgctgtc agggccacag ggaggatgtt tagtgcacaa aggccttccc |
| 38521 | tttgcctctc atggcatcac accattcagg cagctgcttt tggcacccac aagcaaggtt |
| 38581 | ggtgagacac cccccaccag gggcccccaa ggtgaagcag agccttcttc cctggggtga |
| 38641 | gataccatga gggtgtgacc tatgaggaca ggagaaacag gaacaagtgg aaggctcaca |
| 38701 | cacagaccaa gtgggtgtga aaaaggaagg agaatcggaa gtcaagcacc agggagggca |
| 38761 | cctgagagcc cctgaaggaa cccaaccata ggtgccaaag catccaggag ctttacagct |
| 38821 | cagccactac agccctagct gtgcaggagg ggaaaccaag tctcagaggg gaacctgagg |
| 38881 | tcacatgggt agacaatggt agagccaggg ctagaatgca tgtctgttaa cttctcttcc |
| 38941 | taatccaact cccagaaacc aaaatatttg agcagtgatg gtggaaactg agggcatgga |
| 39001 | agacattgtt aaatcattta taatgaaagc caccagccta accactgaaa tgctggaaac |
| 39061 | acttgagccc atcaaaagac tgctacaaac aaatcagaag tatttgggaa atcgggaaat |
| 39121 | tgactcccat gcagtgaata tgttgaaagt tacagcttct attaggtgtt tttggttttt |
| 39181 | gttttgttca gagacagaat ctcactctgt cacccaggct ggagtgcagt ggcctagtct |
| 39241 | cggctcattg caacctctgc ctcctgagtt caagtgattc tcatgtcttg gcctcccgag |
| 39301 | tagctgggac tacaggtgca tgccaccagg cctggataat ttttgtattt ttagtagaga |
| 39361 | cagggttttc cccatgttgg ccaggatggt ctcgaactcc tggcttcagg taatagacct |
| 39421 | gcctcagctt cccaaagtgc tgggattaca ggtgccagcc accaaatcca gcctgtatta |
| 39481 | gatgttctta agtgccaata agaaggaaga cagaaagggg aatagttcct atcctaactc |
| 39541 | tcctcttctt ccctccctgc ccccatccaa gagaaggcag gaaaccccac aaggggaggc |
| 39601 | ccaggcctgg tgggagaggt tctggcattt ctttggaagc ctagagaatg cccagggcac |
| 39661 | tgctggctgc agagctgaga tgggaaagaa gaggtcccct tgtggagagc acaggcccag |
| 39721 | gctgacacag gccagcttgt gggggtcaga gtgggagaga ggtgtttgga atgatgtgag |
| 39781 | actcaagtct ccccatcctg gcatttgcct gggccaggcc agaggagcaa tgaggccact |
| 39841 | gccctcaacc cccacatggc ccagttcaga ccccaggctc cttctcatgg ggcagctgag |
| 39901 | tggaaggggt ctttggggtc tgcactaggc ttagcaatga gcaaagccat ggccactgct |
| 39961 | ggggacagtg cctggcacat ggcaggctcc tgattatttt gtggaataaa agaatgaata |
| 40021 | aatgcccctg tggtgttagg aggcagaggg taggtataca aggtgggaga gcctttgaga |
| 40081 | gggaatcgga gtggcccctc ttctctgaag gactcagggc actcaagggt gacactcagc |
| 40141 | atcacatgac cgatgcattt ggggaaattg agctaaaaga ggcatggagg agctgcctgg |
| 40201 | gagagtgggt cccaggacga gaaatgagcc tgatcccatc actgccatct cagagagaga |
| 40261 | gacttgtcct agaagcagag agtagcttgg aaactggccc ctcacccagt ggttctagag |
| 40321 | gctgccagga ggcagagaca tggtatgaag gaaggaggca tcctaggcag aaaccaggag |
| 40381 | acctccaggt ctgcttggcc ccgtttggaa caaagacctg tgtccaggaa ccttgggctt |
| 40441 | ccccatgaga aagaaatgtt cggttcagaa gattttgtgc atccagctct gcctgtccgg |
| 40501 | gacctctgag ggtccatggc ctggcctcca gcctcctgcc tgctaaggat gccagggacc |
| 40561 | ctgaataatt caggagccag gacagcagga gaggcctcgc agggcccagg acacctgaca |
| 40621 | cgggctccca ggggaaagtg atgaattatt gacaggattc tggtgcccag ttgccgctct |
| 40681 | attgtgatca gtctgtgggc ccatctgggc tggaggcctt ttcctgccgg ctaattacgt |
| 40741 | ggggatctta gctctgccca gccgcccagc ccctcgtggg gccagacagc ctcctgtgag |
| 40801 | gggtgtcctc tttgccatgg tgacctggat ggccctgtaa ggcaagtagt cagaggacat |
| 40861 | ggagttagcc ccagatcctt tgcctgacag ccccagggaa actgaggccc agagaggcct |
| 40921 | cttgtccagg gtcacacagc aggttcagga cagtcaggtc ttcaccccag gcctcctgac |
| 40981 | acccgggcca gtgctccttc ccacagaaca catggcctca caactccatt tctagggaaa |
| 41041 | tgactcagct gtctaaggag catatgagcc cacccttcct caatcgtcct gggccccact |
| 41101 | ctcccaggca gctcctccat gctgacccat gcagaggagt aagaacctac tatgtgtgca |
| 41161 | gccaggatgc acaaaggcca gctgcggcgg gcagggtcgc ctctggggtc accacaaggg |
| 41221 | ccagagccag gggcttctcc tacctcttcc tgtcccagct tcttcccaaa gaatatctat |
| 41281 | agaagacggg ctgagggatg gcatcctaga agcagtgcca gggcctttgt tttgaaactg |
| 41341 | ttttcatggc aagcactgtg tttttgctta aatggtatgt tacagacctg tagaaagagt |
| 41401 | caaggtttat aaaactgctt ttctttatac tttacataag gctgagataa tatcaattgt |
| 41461 | ccatgtcaaa aaatataatt tttactttta tttgagagag gttggggggt gaactgttag |
| 41521 | gagttaaaac cccccaagcc actcttggag gtgtggcaaa ttgattcctt taatcttgca |
| 41581 | agctcctagc gtcccagtcc caccctccag gccatagaca agctctggct gtgcgaggct |
| 41641 | gtgcacagct tgaagggtgg gggactgcct ggacctgccc ctgaagccca tcctgcaaag |
| 41701 | gagactcgcc ctgtgtaggc tctccagatg gccagccctg ctcattatta gggagggagg |
| 41761 | aggggctggc atccgagtgc cggtgttggt agaggaattt cacatgagca gtctcctcac |
| 41821 | cactgtccgg tggaagtcgg agggctatag cagaaggagc caaaaccaca ctgctgccag |
| 41881 | gaggtggaat ctcccttggg tcctgctgag tgagcccaca tctgtctgac tccagagccc |
| 41941 | aggctcctca ctgtctacac cgcttccctt tagggatgga attaatctgc atgtggacag |
| 42001 | ggcttggacc agggtaggaa gagtagggga tttgcctgga ggccaatctt gctcagcaca |
| 42061 | ctgtgtgatt aaggcagtgg ctgaatggac taccagtgac agccccatca ctgtcaccac |
| 42121 | caccggagta tctagcaggg acttaaaagc acatgtcagg gaggttgttt cagggattcc |
| 42181 | tcctggaagg ggcgctggac aaggtctctc aggttcttcc caagtataag atctgagatt |
| 42241 | caggcaggtt aagcctagaa ggcaccagac gcctctgtga gcacttgcag ggggaatagc |
| 42301 | tccaccctgc cccctgggag tgtcttgtca cattgcagca cctctgcgga tcctggggag |
| 42361 | ctgccttggc tgttcttagg catctaattc tggagaaaga tgtgcctgat cgccccacgc |
| 42421 | tcaacaggtg ctcgacaggt cctaggggaa tcaaggcagc tgaatagcca tccacaaagg |
| 42481 | gtaatagtag agcagggatt ctcaaagtgt ctccccagcc agcagcttca gcatcacctg |
| 42541 | gaaacttgtc agaaatacac attctcaggc cctctcccac ccactgaatc agagccctgg |
| 42601 | ggtcggggcc agtggtgtga actgaagtcc ggtggaccca gtttggtgac ttcacattag |
| 42661 | aagcttgaag ttggccatgg cgggagtatt tatatctcag aaaccagcaa atgctacaga |
| 42721 | tgagcacctc tcctctccca ccagtgacac attttccagc atatccacag aagaggatat |
| 42781 | cccagccgtg catgttttca caagccctcc aggtgattct gctcatgctg aagtttgaga |
| 42841 | accactgatt tagaacaaaa aggggtgagg ctgcaaaaag atgagtgtat tttttcgtgc |
| 42901 | ccgtggtcta ctgttagaat gtctacatgg tacggaagct aaaagtcccc ttccctcttc |
| 42961 | attttataaa gaccaaattg agactcaggg gcagcagctc cagggctcct ggcccccagc |
| 43021 | ccagcacttc tgtctctggg gcactgcctg agctgggaga ggtgcatgca gctatggaag |
| 43081 | aaggcttcca tgggctgctc taagggccca gaagcccagt tcggctttag agtgaaggag |
| 43141 | tggcgtggtc agtttcatcc tcagggaggt ggggcacaat ggtggttggg gctctgagct |
| 43201 | ccaaagggga gcatttcata tccattcctc atggctgccc ctaccacttc ctcccctcac |
| 43261 | acagatggga agatttttaa aaaatcaatg ttcaccctcc acagacctca gggtggtaga |
| 43321 | gacatgaagc atgtctgagt cagaattcat ctgatacctc acctcaccca gatctaccat |
| 43381 | taattagctg tgtcccttcc ccttccatgg cctcactgtc ttcctctata aatctcaggc |
| 43441 | agcatcagat gatgcccaag gtcccttcca gctctcacac gccctggatg cttccctccc |
| 43501 | ctttgtgcct tcagctagaa atggtgtgtg tattctaatc cactctatgt ggaagcacca |
| 43561 | tgtaagaacg tctgtctgag ttccctgttc tctcccagag caacaaacag ccttctgact |
| 43621 | gtggggtgca atggggttga agtcaactcc atcagcattt attgggcacc cactgttgtt |
| 43681 | cattaattca gccagcagat attgagatct ctagtgtgct agaaatgtca aacagaggct |
| 43741 | agaataaaaa gcaccaccca tgcccttgag aggttcacag tttagaggca gtacatgaat |
| 43801 | cacgacaaaa cggactgctt tggagtgctg gctgtgtgct gggggctttg acaagaacac |
| 43861 | tcgcagatga tctcattcag tcctccaaca acaactctgt gaagtagggc ttatcagctc |
| 43921 | atcttatgag aaaaccgaca ctgagagagc ttaaggaagt tgcccaggaa gcaggtagct |
| 43981 | tgctagtcag tgtcagagct gggttcagat ccctgtctct ttgatgccag gactgtgctg |
| 44041 | ttaaccaaag cattgcaccg cctctcccct gctgtgcagt tccatgggct ctgccatcac |
| 44101 | acaggttcct gccatccagg aacttacagg ctgcagatgt ggaatggatg agtgagtgct |
| 44161 | gttggtgtgg gctggaaacc ttggtcatgg ttaggcgggt agaagccgga ggagctactt |
| 44221 | gtccatccag gaaactcagg tcctttccca gcctccacac tgagcatctg gaagcttctc |
| 44281 | tgcaagggct cagctaaagc tggatttcat gcacctttcc ccgcagtctg tagacatccc |
| 44341 | ctaagggtgc tcactgcccc ttggagctct cccaaggaaa tgggcctccc acaaacaacc |
| 44401 | atccacagtg ggacatgctg ccctggctgc aaccagtcct ggctcctggg ccaccttctc |
| 44461 | cagcccagcc cagagctgga gcaagcccac ctgagtcctc cccaagcagt cacagccctt |
| 44521 | gggatgtcag tcttgctcct caccctggtc tcttccatgt taccaccgtg ccccctctgc |
| 44581 | catccccagg ctcatcggga ccttccgcat ggtgctgcag aaggtggtag aggagagcca |
| 44641 | tgtggaggtg actgacacgc tgattgatga caacaatgct atcatcaagg tgggtatgcc |
| 44701 | ccacggagtt cctggacttt cccatatggg gccatctggg ggtctgttct ccctgcctca |
| 44761 | cacatggggt ggtctgggga atgcctctca tgcatggcga ggtgtcgcag gaggaggaga |
| 44821 | ctcaggtcag actctgctgg aatggctcaa tctgtggcca tggctggagg ccctgtggca |
| 44881 | tggcagggtc tgctgcccca cagtaggcac ccctgccctt cctacggtgg gaatgcacca |
| 44941 | actggtcctt ccatttggac ccagctcagg agtcactaaa gaggctcctt ttgtcatcca |
| 45001 | aaggccagac tgagccctgc tggcactcga gggcatgacc ttcactctgc acatctggct |
| 45061 | cgggtcctcc ctgggagggc agggacctca tttcctattg tttgtcacat cccagcactt |
| 45121 | ttgcgtgagc acgcgtgcac tagtgatgca cagtctagag gcacctgaca catgttctaa |
| 45181 | caaagaagga agcaacccta ccaggacagc ctggccccag gctctgctct gattcttcag |
| 45241 | tcctcttgag ctcagccgtg gccactcatg ggcagttggc atgatggtga ggaacaggga |
| 45301 | ccctgagtat gaatcttagg ctgctcacta actgtgtgac ttaggcaagc tacatatcac |
| 45361 | ctcaaagtga gcacaattac attatccacc tcccaggact attctgagga ttaaaggaca |
| 45421 | atacacagaa agtacttagc acagatatgc acaataaatc tataatattg ttagctatta |
| 45481 | tttcctgacc tcagtttacc tatgtataaa atgggactgt gccatggtcc cccagtgggg |
| 45541 | tcctgtgtaa tatatggccc tatgtagtgg ccaagagaaa cagcctcaga agccaggact |
| 45601 | gcagccacag ggagctagaa cagaaggaac cctcttctcc catgggcagg ggtggcaggg |
| 45661 | agggcttctt aaagttaccg gggtctcagc caccacagca aggggagaag gggtgcagaa |
| 45721 | taagctatgt atggacaccc atgggaaggc gggctgggag gagaacacag aaacctggat |
| 45781 | gaagagtgta gtgtgggaag tgagataaga gaattgggga gtgagcaggg atggacagac |
| 45841 | agcacagccc atgccctgga ttttcagaat cagacttaag atgtcagcag aggaaaggac |
| 45901 | tcacggatct cctagcccac ttgccaccca tctccgctac ccattgttat gaatgggaac |
| 45961 | aatgaagtcc tggatgggga atggattcgg ccacagttga cagcagggcc agcacggaga |
| 46021 | cccaggactt ccaatgccag agaccaccat tttcccataa caacatactt ggttccacca |
| 46081 | agagggtccc atttgttcca gaaaggacat ggggcttgga gttaggcagc ctgggctcag |
| 46141 | atcccagctc cagtacttcc tagctgtgtg acttaaagca agtaacttaa cctcctgata |
| 46201 | tgctaggtaa ggtaggttgc aatgttgaaa agatcaaatg agtgtacatt aaagcatctt |
| 46261 | agcatgatgc ctagcacagt gaggattcag aaaatatgtg cacctttccc ttcccttggg |
| 46321 | aaatctaagc ctgggtgctg gagcatccaa gcactgagtg agggccgcct gtctggggca |
| 46381 | caggaccaag tcacctctag accagtgagg aagggtgatc cctgtgcact gggggggaaa |
| 46441 | ctagagctgc aagaaagtaa tggctccagg acgcagagca ggaagcccaa tcatgactct |
| 46501 | ccactgctaa gcctgagccc ggttgagtgc tctacttttt gccagaggaa aaccctaggg |
| 46561 | cagctcccaa gattccctcc ctctccaggg taggaagggt gagtgtgagc ctgggccctg |
| 46621 | ggctcctctc ccggaggcca ctttcagctc attctgtcca tttccaacaa gccgatttct |
| 46681 | ggcgggccgc gcctcatgcc tgcaggcagc ccttgttctg aggagaggag gtggcctgat |
| 46741 | catggggatg ctgaacgaca ctctcggtta tgtagacagt ggactcgggg ggtgagtcca |
| 46801 | ggctcagttg tcctgggcag gccaggcccc tccaccttga gcaccaggga ggagtggaac |
| 46861 | caccagcagc gggacctgga gccaggtacc agtaagctga cccggtctgc acaagtcagc |
| 46921 | ctttcagagc ctctgttttc tcatcagcaa aatggggcag aggggatcat accatcctga |
| 46981 | cctcctccag tgaggcaagg gtgtggaagc gctgtgtaaa caccaccaag cagccacata |
| 47041 | tacggggtgg agattgctgt caccatccca gctgctcacc accgcctccc tcccttcaga |
| 47101 | ccagcctgtg cgtggaggtc cggtatcagg ccactgacgg cacagtgggc tcctgggacg |
| 47161 | atggggactt cctgggagat gagtctcttc aagaggaaga gaaggacagc caagagacgg |
| 47221 | atggactgct cccaggctcc cggcccagct cccggccccc aggagagaag agcttccgga |
| 47281 | ggtaacaggc aggacccctc tggctgccca ctgctcccaa gacacgggga agagacctgg |
| 47341 | ggcctggtcc tactgacagg cctctctagg cctagcbggg ctcagagtac agggctgagc |
| 47401 | ttctggagag acatccaagg ggtgggaaga tgtttctgta gccccccacc cagccgtcca |
| 47461 | cccccgaggt cactgggcag ggagcaggag actcagatgg agctgcaagg ccaaggcctc |
| 47521 | cccagtggcc ctgagggagg tggagctcta acacagggat ggggctagat ctcctgcctt |
| 47581 | tctcagagaa ggggagttgg ggataggagg gcccctggcc tctctggttc aagacccagc |
| 47641 | agtgtccacc aggtgccggc agcctgacct ctaccctggc ccagtgagga ggcccaagag |
| 47701 | agggcagctc ttcctgccag agtgaagcca aagatcctgg ctgggccctg gcctttcttc |
| 47761 | ctcttgagaa gggagggtac ccgggaaccc ccagaatcca gcgtcacgcc ctgcaagtgt |
| 47821 | cctgccacct atggatcaca ggaagctgga tccaggacag ctcattccac gtgcactatt |
| 47881 | tttacaaaac agaaaatgca attgtattac tattagagac caaggggaca gaatctgggc |
| 47941 | tagtctagta agatgttgtc catgggttcc taagtcacta ggaattatgt gttaaatgcc |
| 48001 | agtaagtcaa gggtgcatct gaaggaccag ggtgtgtttt gagaagggac cctccgtgcc |
| 48061 | actggaaaca tatccctgta agctccttcc acgctttcct ttccaagtaa agtttactca |
| 48121 | gtccctctgc tgaagaaacc ataggttcta aattagtgta attgaactga catactttta |
| 48181 | tgggacaaat atgttttctc ctttgcactt gaatgagttc acaccccctt aagcacccct |
| 48241 | cacacacagg tgcatgagtg cacacgtgtg cacatacaca tacacacaca cacacaagct |
| 48301 | tcagaaaccc tgggagccag atgagtaata gtaacaaggt caaagtgacc aggcctgcaa |
| 48361 | caaacatggc gggactgtct actccaggcc agccacgggc atggggcagc cccaggctag |
| 48421 | cttaaagcct gtcccctcag actcctggct ctttagcaga aaacccaggc ctgagtccta |
| 48481 | gctctgcctc ttactagtcg tgtgaccctt ggcaagtctc tgaccctctc tcctcacctg |
| 48541 | caaagaagga catagcaagg aaatccccct gccctgctgc tgggagagcc ggagggatca |
| 48601 | cagaggaaca ggtggcaatg gaggtgctga gcgctgccca tgtccaggct ggtgacagac |
| 48661 | aaacaggttt cttgtagcac aacaatgcaa agagtcagga atcgggttgg aaattgtgag |
| 48721 | aagaggagca gtgctcagac ggggagtgaa gggatgctga ctcagtgggc accacctggt |
| 48781 | cctcctcgac tgtctccatt cagaaaagag gttggcaggg ctggtccaga aaccagaagc |
| 48841 | cgggctctaa gaaagagctt ccagagaaca agatcggaag ccacacaagg gccatcagtc |
| 48901 | acagggaaag gaatgctgac cccaaatcag gagagctaag gcccattaag tcccatcttg |
| 48961 | actgtgccac tcattggctg agtgacactg ggcatgtcac cagcccctgt aggtcagtga |
| 49021 | caggaatggg ttcagcaagt tacatctggc tcccctccaa ctctgaggtc ctaagggacc |
| 49081 | catggatgct ccggacctgt gggtcacatc catgggaaac aacatcctgg gagcccttag |
| 49141 | cagccacgaa ggcaacctcc ctggggtggc gtagcaacac agagccagtt tctcactcag |
| 49201 | tcctaagaga gtgggccagg gtgctgatgg gggctctgga gagcttagag tcccagagag |
| 49261 | ccctaggcac caggaatctg gagataagag ggctcccccg acccttcctc acagcctggt |
| 49321 | ggcttcccca cctctcccca tcttcgtgtg gcccctccca gggtagcagc aagaagcggg |
| 49381 | agcaaaagga ggggcttcac cccggactgg gaattgggag agggtgacag ccgaagccag |
| 49441 | gcacccccac cctgcattgc aggggcagtg tgcaggagca cagtttccct gctgccactg |
| 49501 | gggctgccag cccaggtttg agagcacatt ggggacaggc atcgctgcta caccttctcc |
| 49561 | cgccaggcct agccatgcag ccagcaggcc tggttgtgga aaagaggggt tgagaaaaat |
| 49621 | tctagagatg ggccatttgc tggtgaggag aaggtggagc gggttcaaga acttggggtt |
| 49681 | ttcctgtagc ttaatcctgg ctgcccatat ggggccattg agttctgggt caaggagtgc |
| 49741 | tgaggtgagg agggggacct ggaagctggc cccgcagccc tagatgttgc atctcaccct |
| 49801 | tgtcacatag tgtgggttct gggaggaagc cagagagctc tccagaaggg acttggccaa |
| 49861 | ccttttatca ggaaaacacc tactccttaa agaggacaac accggggaag gggtgtcagt |
| 49921 | gcctcacctc tgaccccgtt aggagcccag gcaggcttcc cctgcatctg ctccctgccc |
| 49981 | tccatcctct cctgggaagg ccaatcccct tatagatcag ggtattcaat aaccacagtt |
| 50041 | ccctcttgtt cctgctttag ccacaatgag caagaggaaa atcccgttgc tggcaagcag |
| 50101 | cagtgtctca ccggccatct tgaccgttag ggtccggcgt ggggatcagc cgctccctgg |
| 50161 | ctgatggggt gaaatgaagg cctgctgggg gctgcgggga ggtagactga gtgggcagga |
| 50221 | aacagcctca gcccagacag gccccgggca gcccaggctg gaatcttcca cggatggagg |
| 50281 | ggaggctgat gccaggagat cctctgggca ctcctcacct ctccctccag ggcctgggtc |
| 50341 | caccccccag ccctgccctg cctggcctgc ttctctggga cttcacccac attcatcctg |
| 50401 | tcctcactct gggggtcaga tggttctaag caggggcagt gtggacctca ggcctcagct |
| 50461 | gctgtgacac tcactcctaa gaccctaaga atctgccatg aagttacccc gttcctcctc |
| 50521 | agggaatcag tatgggggct ctgcctgaat gaccaccaac cccaccgccc actgactctc |
| 50581 | tgccccaacg attccacctc ccctaggccc tggtcagccg catgccctct gagccactct |
| 50641 | gcccacttct ctagttcctg ccacgaagcc ctgatttctg ccggcttttt gcacaatgcc |
| 50701 | cccggtaatg tgccaggaag ctgtgctgtg tgacagagca caggctccac tcacagctct |
| 50761 | gctattggga acccccctcc ccatttgtga gatggggctc aaggatgcct cacagaagtg |
| 50821 | ccaggattga gtgaggtgca cctgcgagtc atccacccct gtgcctggca tgtggcaggt |
| 50881 | gctcagccag tgttcacttc cttcctcttc actgactcca tgttaccctg atgtcaactt |
| 50941 | ctaccccgag cagcacctga ccctcttgtt caggtgctgt ccaagcacct tctcagctct |
| 51001 | tgctccccac cctataccaa gggctctgca gaagctgccc acacaggagc cttactggcc |
| 51061 | caggggcagg tatagggagc tcatttactc aaagttcccc tcacctcccc aggctctcag |
| 51121 | tgttgcagag atgtaatgaa tccctaacct catgggcgct ccactctcag cagcacaaaa |
| 51181 | tcagtgttcc agacccttgc atggcagcgt atgcctgccc tagtctctgg agagaaagtg |
| 51241 | acttcggccc tgaccacact cacatgcaca cacatgtgta cacaagtgcc catgtgtttg |
| 51301 | cacacactca catgcactgc atgacaccag ggaggactct ggattctggg ctctgctgtt |
| 51361 | gatgctgtct ttaggagtca catcttagag aggacaaaga aagaaatccc agctttagtc |
| 51421 | ttgcagctaa actgtagaga cctgagcctt tcctcctgag tcccagtatt ctgcgactat |
| 51481 | ctcctcttct cattttctcc acctggcctg gctctcctgc cctccacgac ccccacctcc |
| 51541 | ttctgctgcc caaaccctct tctgctggtc tcctctctgc tgggtctcat ctgggaggag |
| 51601 | tttggggcag gagccacttt tctcattctc cttattgatc attcttccac tccagtggag |
| 51661 | gcctgcagaa gccaggtaac ttcctcctat gctggggcca gagggcagtg gggcacgtgt |
| 51721 | gcatggtggg gctctgagac agaggggcag agagtgggca tgcagaagtg tgtgcatgtg |
| 51781 | gggccagtac atgcagcctt accccaagag tgtttttaaa tcggtttcca cagcccctag |
| 51841 | agagctagaa aatcctctgc tgccttcctt ctccaggctg agccccagag ttgcagcagc |
| 51901 | tcccaaagct gctcagatga gccggggcag ccgggcgcac tgggattttg tgcttgagac |
| 51961 | acattcctcc tcctatgctg tgagctcttc tccgcttagt agtctcagct gatgtgggca |
| 52021 | gccgaggcct ggctgatgtt aacacctttg aattacagac tgacctgctt gcccaacgta |
| 52081 | aaggttagct ggccagcgct ggactgcagc tgtgctgtgt ctaggccttc aggtcttgag |
| 52141 | ccatcaggat atgcagtcaa gtctacctgg gcaaggcctg tgccagaaca gggtgtgagg |
| 52201 | agaggaataa tttgctaatt gttattcact ttcctgtctt catcctcatt gccacagcac |
| 52261 | tgggaaagca ttgagagaac attccagaaa agttttctgg aagcctccct aaagtgagat |
| 52321 | aggggggccg tttttccctg ctctgagccc tggagtggta gctcacttac cgccccacct |
| 52381 | acacccctag aactgccccc aaccacccgt gtgcactcca tagcaggacc cccagccctg |
| 52441 | cctcaccctc acagagcagg cttagcccac tgttccccta gaggccccca gagcctggga |
| 52501 | gctcccaccc cattggaggg gagcagggga gcaggggagc cggtgggcag aggagcaggg |
| 52561 | tggcccaggc cgggagggtg gccaggctca tctaaccctg tgttccctga ggaagctgcg |
| 52621 | acccacattg tggaatcatc agatcttagc ctggtagggg cgatgagctg aggccctgag |
| 52681 | gtatcagagc ttggggttgg aatagtggcc acccaggccc agcactgtgg gcccctccca |
| 52741 | tctcccccaa ggcagaaccc agggccatgg gacccctttt ctgcagacct aggcttgcta |
| 52801 | ccgtgcgaag ctgtggggtg agaggtctgt gggacaagga ggcccccggg gagcgtgcag |
| 52861 | gcccgtgggg ccggggctga ggccagtgtc tctggtccac agcccctcac acgtgtcccc |
| 52921 | ttgtctcctc atttcttccc cctactcagc aaaggcagag agaagaccaa gggaggcaga |
| 52981 | gatgacgagc acaagtaggt gtcccaggcg agggcagacg caacagcgtc tctggggaga |
| 53041 | cagaggcctc ctccccacag gctgtggagg gcgggatggg ctcggctgag agggctcagg |
| 53101 | agtgggctgt cgtggtagag ctggcacggg tcatggaggc ccagggaggc cttttcacct |
| 53161 | ggccacgggg gtgagggatg aggaggggtt agggtcgcgg gggaggtggc cagaccttct |
| 53221 | ggtttccact ctgggctctc gggggtgacc atcctcctct ccgtgtcttg ttcctcctcc |
| 53281 | actgttcagc ccccatcctg tggccttcgg gcttccctgg gagggagggg acagctaaag |
| 53341 | gctaagggga gaggggcatg aggacctcag ggatccctct cccaacaggg gcgaggtagg |
| 53401 | gccaggcagc tggaggcata aggaggagga actctgggta ccctttgcag ctgcaggtcg |
| 53461 | tgggtggagt tggagacccc cagaaagctt aggagaggcc accatttggg cggccctctc |
| 53521 | tatcttcatc tccccaaaag gcttcctgga aaaatgaggt cagggtgctg tctggagagg |
| 53581 | acaggagaag ggtcctgcag gtgggagggg ctggtcacaa gggtctggaa agattcgaag |
| 53641 | gctgggagtt gtgaatgggt cttgtaggca cccctagaag cacactgaca tctggaggac |
| 53701 | tctagcatta cgggatctca taagggcagt ggtagccaag gtggaccata ctggaggagg |
| 53761 | cctctggtta acacctgctc ttacagatga caaatctgtc tcagaacagg ggcagagctg |
| 53821 | gaaccagaag acagggaagc tgacttctga cccagagctc accatagacc caggaaattt |
| 53881 | agagaacttg actgtatcta aatttaatct aattctccaa acataccaat gagagctgaa |
| 53941 | aagccaggag tgtggtcttt tttatcttcc agataagcaa cctgagcccc agagatctta |
| 54001 | agagatttgc tcaaaactgt ggcctcaata tattaagaat tagttcatct gaagatatca |
| 54061 | tggaaatatc aggattgtgt gttgaggggt ctgaactgat caacactggt gtgggcttgg |
| 54121 | gatatccact ggcattaaga tcctggggaa ctcacagcct cattcattgc ttcaaaaaaa |
| 54181 | ggtatgagtt ctaactcgca tttgttacgc acaagcaaaa gcaatgatga tgcagtgtta |
| 54241 | actctgtagt gatggaggaa atggcgctgt gtaggaagag ctccagccca gagacctgaa |
| 54301 | ggatggagga gtttgccagg ttcccagtaa gggaagacac gtaggcagag catgtgtcaa |
| 54361 | ggctcagagc cagggaactg aatccagggt gggtggcgat gatggggaat gctgagctgg |
| 54421 | ggagtcctct tagggagtcc tcaagggttc aggtttctcc atagatgctt agatgtaaag |
| 54481 | ggcatatgaa tgagatacaa gcagcctgtc catccccagc ttctctgcct gagatcagcg |
| 54541 | atgctctaag gcccttcttg atgcccagct tcccagtctg tcccaggcca gacccacagg |
| 54601 | gacacagact ggcctgatcc ctttgactgg ctacagttgg agtaagaggt acctcacagt |
| 54661 | gccagcacat agtaggtcca ccaaaatact ggtttcctag cacttctctg tcctaatttc |
| 54721 | ttaccatctc taattagcac atgagataaa gtaaactggc atatagtatt gttattatta |
| 54781 | ttatgtgtag agcaatagga ctattgaaag aataggaaaa tgagatttct aagaagagat |
| 54841 | tgaacaaatt taaactcttt agcctggagg agagaaaact gtggagacag atttcaatct |
| 54901 | ctgaaatgtt aatgtgtggc aaagctggtt ggtatctatc tccactgaga agagataaat |
| 54961 | ggaaataaat ttaagttgaa gttaagttga agtagagaaa caagattcaa cccacgaaag |
| 55021 | aactcatagt gtgtgacatt ctgggatagg ctggtttggg gagatgagaa aacacccatc |
| 55081 | atcttccatg atgctgcttg cttgagcctt gcaaagggct gggccgccag caagaactga |
| 55141 | agagccagct tccctgacaa tcctggggtc aggacaagtg tgtgtgggtc ccccaagcct |
| 55201 | tctggtacaa cgtgggcaca tgtgcaaacg ggccagtttg gagacagggt ggttcttgta |
| 55261 | taaagggtca gcctatcctg cccacctatg gaaagctatt caaaaactaa ctgacattca |
| 55321 | acacactttc aaagacacct gtaaagacca aaattatgaa attccctgtg ccttggggca |
| 55381 | tggaaaattt ttaaaataaa cccatttagg ggcgcttgga taacatgctc cgaggttttg |
| 55441 | aaaattgggc tgtttgagca atgcaaagaa ggaccacaga ccctatttct catagacagg |
| 55501 | agtctttatc ctgattgatt acctgaggcc tgagctaatg agcctagtag ttccagttga |
| 55561 | agccagtcat ggggcagaag ccctgggcag tgaatggact cgacctgcag ctcatttatg |
| 55621 | gtcaggtctc tattagcttt cagaataatc cagagacctg aattaatgaa acactcgggt |
| 55681 | cactaaattt agtttggtga atgttcccaa gtcattatga atactaagct ctacctagga |
| 55741 | ccctgtgtgc accacacagc aacacgcttc taaatctggg ccataaaatg agcggagaaa |
| 55801 | tcatcagatt tatagtttac aataagtgaa ctaattttag catttttttc ctctagagat |
| 55861 | aaataccagt ctagattgta ggtgcatttc attctgacca tggtgaatgt agccacagga |
| 55921 | aactcaaacg gattctaaat cctcagaggg tcctgggata ctggcctccc cttggctctc |
| 55981 | acttctcaag ctttaatacg cccaagaata aggagccatt tgattgtaaa gtgagttttc |
| 56041 | actgagttga ttattggctt gttttctttg tctgtttctc accatatcct gtttttggta |
| 56101 | attgtagaac tcaactagac caaatgcaga attttacttt gaaaacaaaa aaaggccagg |
| 56161 | acaacggaca cctcaacagc aaattacatt ttttgaaatc taagtctttc agacaaatag |
| 56221 | ataatcttca aaattccctg attaagttga catttatttc aaaactctcc tttacagttt |
| 56281 | gccaagagaa gtaggcatac tgctttctgg gaactggtgc tgcaatcagg cagaaaggct |
| 56341 | ttttctatag tgaacttctg atgtgttact gatcagaaaa ttacaaaggc tagtggcagt |
| 56401 | gatgacatca tgctgtactg atcacaattc agattcattg aagagcaagt ctaggagaat |
| 56461 | cagaggtaca gagcctgtag taagaattct ggaaattaca tccttcacta cacatttctg |
| 56521 | catttttaat aaagggacaa agacaatatc cttttgagat attcccaggc acacaggagt |
| 56581 | gctaccagta ctaccacttc agacagctaa aactatgcag ggttggaagg gagagatttc |
| 56641 | tcctttcaat tgcttcaagg tgtagtagag atagttggac aagtatagtt tttgtttgtg |
| 56701 | tgtttgtttg agacagagtc tcactctgtc gcccaggctg gagtgcagtg gtgcgatcgt |
| 56761 | ggctcactgc aacctccgcc tcctagttca agcgattctc ctgcctcagc ctcccgagta |
| 56821 | actgggatta caggcatgtg ccaccacacc cagctaattt tagtagagac ggggtttcac |
| 56881 | catgttgacc aggctggtct tgaactcctg acctcaagtg atccacctgc ctcggcctcc |
| 56941 | caaagtgctg ggattacagg tgtgagtcac cgcaccggac caggacaagt atagtttttt |
| 57001 | aaagcctcaa tcatttcttt caaatctgtt tgtgttaaga aataaagtga ctttcaaatt |
| 57061 | caagggccat aagtgcccca gggatgcctc aggtcctttt ccacctgggt gagtccatgg |
| 57121 | gaccagcctc tgaaagccac tatttcttgc taccttgcac tccccccggg actcaggcag |
| 57181 | cagggccaga tcccatccca gcaggccggg cagccggctt ctccaggcag ggcctgcttc |
| 57241 | taacctacca tctcagagca cttcccctct ggggaagtgg ggaaggggca ggggagaggg |
| 57301 | aagtctgtgg ctggagcagg ctgcaatggc aaaggctaac tatgtaggtc acacatgcgg |
| 57361 | aagggccaag gactccaaga ataacacagc ctaggaatgg ccaagaagta atggtgcaaa |
| 57421 | gtcaaacgct gagccctgca ggagactcct gggtaattca ggagagacat agttgagcag |
| 57481 | cacggctggg caaaaaggaa tcccaagcac tgagacagga agtgacccac gaggaggccc |
| 57541 | acaaaaacta cacttctgaa gctgggccta tggcaggggt caggacagcc agcacccctg |
| 57601 | tccttggggc cagcaggtgg ggcaaaatcc cacaggatgc ttcagggcaa gccagccagg |
| 57661 | gtggtccttt ccccaccatc acacgattat ttctggtggg ttcattgatc tgttcaatgt |
| 57721 | tatgtcacca gcaatttaac atgcacagca gtcccttccc aaatgtttgg gtgctcaggt |
| 57781 | tctttttgaa tgagcccctt ctttgtaaac atgggtatga tctgtgagat tccacagatg |
| 57841 | acagcatcct gatctgtaaa ctatgctggc acacagacca tacctgtacc tatgggtgcc |
| 57901 | agccctgggg ccttttagct aaaatcccaa atggtcagga atggatggga ttcttgccca |
| 57961 | cagaaaagtg cagtcctacg gcatcgaggc aaaggacctg agatgcagta aaaatattgc |
| 58021 | tggcctagaa cacaggagct ttctgcttca atcccagttc tgccactcac cgtgtgacct |
| 58081 | cgcgattatt gtcccctctc tgagcctcag tctttttatt ggttgaatga ggagaagtat |
| 58141 | gtggccctca gaggattgcc aggaagatga aatgagcaga catctgcaga cattgcagac |
| 58201 | atctagaaag gcctttggcc ctatgtagtc tctttttggc ctgtatgtgt gtgtgtgtgt |
| 58261 | gtctgtgtgt gtgtgtgtgt gtgtgtaact cacctccccc atcagaaggg aggaagcttt |
| 58321 | tgaaaataat tccaaaggca tcagctttga ttttagagcc tctttctttc cctttcttct |
| 58381 | cattgtcgta tttcagatgg tatgctactg aagagtgcag acacatcagc cttcttccat |
| 58441 | tcctttttag gaattcaaag aacatgctaa tgttccctaa attgtgagct gctgccacgt |
| 58501 | gaaaagggtg attctcaatt gctccctcac tgggatatga acgtgctttg tcctgcatat |
| 58561 | gaacagtaaa gaaaggtcct ggggttggga tagggccagg tttggggaat aagattggcc |
| 58621 | ccacaatcct gaggagaggg gcttgaggtt tctaggtcat ctgcctgagg acagaaaggt |
| 58681 | tttccccaac ctcctattgc ccagcataaa gaacaaggat ctggggtctt agaaaccata |
| 58741 | gtcaagcctc cacagggaga tgtgtgaccc aaaaagggtg gactctgctg taagcgagct |
| 58801 | ccaggttgga gcctacacct ggctccacct ggacctacag gatgatgggg ccatacccag |
| 58861 | gaggagggag aacgggaagt ggagagagtc aggtatccac tcatgcaata tggatggggt |
| 58921 | aatggaacac tgcactttac cctctgctga tcctgctcct ggggcttcct ggtgccagga |
| 58981 | accacaggct cggctgcagg gacatcactg aaggacagtg aggacccctg ccccttttcc |
| 59041 | tcagccccgc tgactgcaag gcccttcacc tgggggcaca aggtcgcagc aagccagagg |
| 59101 | aagaggtagg aactatggag cctgccataa gttcctgggg ttgggggaaa ggggaatttg |
| 59161 | gcagtgagag gggacaggta gaaggggcaa aatccaggac cgaagataga tgggaaaggg |
| 59221 | cctatctccc cctttttcat ggggaccaag gagcatccca gaacacggag aggttggaga |
| 59281 | aagacaagtc aggctttgag caccagggcc atctgagaac tgaacagctc gtttcaacca |
| 59341 | catctcccat ctcctcatcg gcctctgccc ctgagtcctc tctggtccct gccccgccgt |
| 59401 | tctgatgctc tgtctccagt cctcgggctt tctctccaac cagctgagcc ggggaggatg |
| 59461 | agcgtgggga cactggcagg gctcggagct ctgaggagac tagactgagg ggctgggatc |
| 59521 | agactgaaaa gtccaagacc cagaggagct ccagttaaaa cggctctttc cggctcaaga |
| 59581 | ccacgttccc tgcttgctgg ggaccccatc cctctcctcc gtgtgtgaaa ggatggcaaa |
| 59641 | ggcggaagtg gaggggtctc tcactgccct gattccccct cctggctccc aatttgggaa |
| 59701 | gacagatccc gatctgtctc gggaccagta ggtgaggggc cgggtccatc tcccttctct |
| 59761 | gatgtgttct ctcatgtttg gctcttctgt gtttgtgtgt tttcctccat gcgtccctct |
| 59821 | ccctgcacct cattctggtg gcccccctca cagagccggg aggagcgtgt tctccgccat |
| 59881 | gaagctcggc aaaaaccggt ctcacaagga ggagccccaa agaccaggta ggacaggtgc |
| 59941 | cgcgtgagcc ccgcagcccc gcgcctcgcg gcgccctccc gccgctttcc agcccctgcc |
| 60001 | cggctgccaa aaggccctcc tgggataggt cgtcccaggg ccaggcccca cccagtgatg |
| 60061 | cgtggccctc taggaaggag acgccctagg gccgagggag ggaaacgggc agcgccctct |
| 60121 | gtcgggggtg cctggttaca aggctcctcc cgccctcgtt gctcccggtt cttccttctt |
| 60181 | cacgcagaag cccagtgttt taattagagc agagaatatc catcaagaaa cgctgtttgg |
| 60241 | ccaactccct atttctgggg ggaaaccgtt gtcccactag tacggggtcg gcggcaggcc |
| 60301 | tcgcccaagc gctctcacca ggctgcagcc tggcctttgg ggaggtcctg gggccgctgg |
| 60361 | agggcgcggc agggccgggc ctgggaggct ggagcagcag gtggcactgg gccctcccgg |
| 60421 | aggagcgact ggagagggga agacagctgc tgcagaggag gcgggaggcc tggaggaaga |
| 60481 | caaggggaga aatggagagg acaggggagc tgggaggggg ccatcttctc catcacgggc |
| 60541 | ccaccatcgg ggctcccggc tgccctcccc cagcatcccc tctgtggcct actggccctc |
| 60601 | aggctctctc ccccaccctc ttgcccccat gccaattcca gccgtgccct tcaccttctt |
| 60661 | aaatggagag agtacagctg ctggctgagg accaaaccca gcatccttgg gtggctgttc |
| 60721 | ggaatccaca tgtctgcatc tcattagatg aatacacacc aatgcattag gttctgaatt |
| 60781 | ccatcagctg tttgatatgg ataagttact tcctaagcct tcgtttctgt acctgtgaca |
| 60841 | tgagaataac gcccatcttg gcagggcaca gctctaagga ctagagatag tcatgtaaag |
| 60901 | tcctggtgtt gtgcctggaa cagagagtat ccacccaaca aatggtagct atttattgag |
| 60961 | aggtgcaaaa gtatggattc tttattccct accttcttcc ataagagata ttcagtgcct |
| 61021 | aagtaagtgg tatacaataa aagagcattc aattaaaata aaaaggcaag accagacata |
| 61081 | atggccattc aaatccaaga tcgggattag gtagaaaaac actgcagtta ttcaggccat |
| 61141 | aatattctaa gcggtagcaa ctaagtaaac ctcgaattta actctcagct ttctggatgc |
| 61201 | cagagtaaaa ggagaaatgg tcactatctc accatcagtc agcccaccta accagtcttt |
| 61261 | catcttcccc agatgaaccg gcggtgctgg agatggaaga ccttgaccat ctggccattc |
| 61321 | ggctaggaga tggactggat cccgactcgg tgtctctagc ctcagtcaca gctctcacca |
| 61381 | ctaatgtctc caacaagcgg tgagtggacg ggagccctgc aaccccagct gcccccaccc |
| 61441 | tgtcaggcag gacctggctt gccatgattg gaatttgggt agggaagaat cagggctcat |
| 61501 | ggacggcttc ttcctaacct aaccttaccc ttttctgctt ttccttgggt gtttttctca |
| 61561 | cctctctccc tgcaaaagca gagcacagca gctgcaggcc tgttctcagg tccttgaata |
| 61621 | cctcccagcc caaggaagcc acagacctgc ccaccccata tttgaaccta ttgaccccat |
| 61681 | gtctgtctct ctgttacttc tgcctttgat tagttctctg tgttgaggcc tctgaagaca |
| 61741 | actaatcctc actattcttg gcagtgcttg agtgtttaaa gaccatgatt ctgtccctct |
| 61801 | gcccactccc aggtctaacc actgccagtg tcctcctccc caacaagcag tccccatacc |
| 61861 | ccaggcctga tggtacattc tcttcctcag atctaagcca gacattaaga tggagccaag |
| 61921 | tgctgggcgg cccatggatt accaggtagg tgggcacctg gaggcctcat gtctttcaga |
| 61981 | ctctgctcta gtgggggatt ctagccggat gtgcattatc cactatactg aggcatggat |
| 62041 | ccaggggtca tagagaacaa caagctgaat ataagttccc aaagtaagct tgttcctggg |
| 62101 | aggcattcct agaagtatca tcagtagaga gatacaaacc caaagaaaag cttgagaact |
| 62161 | ctgcctatgc taatcaaagc ccttccagta tttcatgttc agtattggac ttcataaagt |
| 62221 | gagaagcacc tagaaaaacc ataaagagac caagaactac aaaaaagtat tcaaacagtt |
| 62281 | aaaataaggg cctttgaaga aagttttcct ggcatgattt ttaaattgtt gttttgtatt |
| 62341 | atttgattgc tggaagtaat gacgattctg tttatctctg gatggtgaga ctgtgagtga |
| 62401 | tttttttctt ctttttgctt ctctgtattt tctgagatta acacaacttt tttatttgtg |
| 62461 | tgcttctaag accagagaca aaagactgag acaaagagag gttgacagct gtaagaatgt |
| 62521 | gaatgtggcc aacagtgcag gcaggacagg tgatgagcat gtctggtggc tgctgggaca |
| 62581 | ccatctctca ggaagggccc agggaccctc cattgctcca gaccctccct tggaggctaa |
| 62641 | caggactcag ctataaatca gaagactgct gggctttcct aggattgagt tagaatgctg |
| 62701 | gctgctcaga ggggacaggg cttgcaagag atctcacctg gctgccactg cacttctgtc |
| 62761 | ctggggatgt tcggtctggg tatcagcacc aggccccaga atcagagtga gccccatcta |
| 62821 | ccagggtaga gctttatctc tgatgcctag ttccagaccc caagcacact aacccttctt |
| 62881 | tacggacaac cccacctccc ctcactcagg tgatgatgga aaggataaga tggcactttt |
| 62941 | ctatgtaaaa ggtacatctg gccatgtttt ctccagaata tacttcatct cccttcatct |
| 63001 | ttgttttagt tagtttccat ctactcaggc aactgtcttc ccaggtgcct gaaaaacagc |
| 63061 | cccaggcaat gtcaaacgga cctgcccctg tggttttaaa taacttcaag ccattggatt |
| 63121 | ccttaggggt ttgagaatgc cggcccctca cctctgccaa aacccaaaca caaagtgtcc |
| 63181 | tgagacttcc gagggctttt ccaagggatt gggctcaggg ctttgtttct ctgtgtgctg |
| 63241 | agaactggag aattccaaag aacaacgacc ccaactcagg tatcatgtca ctggcctaag |
| 63301 | aggtaaggac acatcaaaaa tagagtcatt attgttcatg aaacttattt ttgaaggttt |
| 63361 | attctatgcc aggcactgtg ctaggagcta tatatgtatt tccattgaag ttaatgtaat |
| 63421 | tcttcaacca cccaaatata ggtgttactg catttatagg taagaaagtg aggccccagg |
| 63481 | aaggtcagga acttgtccca ggtcgttgcc actaagttag ccaaggttag agccagggtt |
| 63541 | caaggccagg ggtgtctggc catagccata agagcaattg acttctctat atcaagcctt |
| 63601 | ctagacagct gctcccttcg ggtccctggt tcatcttcat cttgctactt agtgcttgtt |
| 63661 | ttccaaacct tttcaaactg cctcctacca gagactttta ttaactggac cagacattgt |
| 63721 | cccctccaaa gactttttaa accgaactga acaaaacggt tgttgtattt aacctggtag |
| 63781 | catagtgttt cccgacaagt tcattcacaa ttaaccttgc aaacatttga gtgagagcta |
| 63841 | tgaggatgca aatgtgaaat tattttcact tccaatacat acagtgtata atgcaataaa |
| 63901 | agcaggccgg gtgcagtgac tcatgcctgt aatcccagca ctttcagagg ccgagatgag |
| 63961 | tggattgcaa gaccagcctg gccaacatgg tgaaaccccg tctctactaa aaatacaaaa |
| 64021 | attagccagg catggtggtg gcgcatacct gtaatcccag ctacttggga ggctgaggca |
| 64081 | ggagaatcac ttgaacccag aaagtggagg ttgtagtgag tcgagaccgc accactgcac |
| 64141 | tccaacctgg gcaacagagg gagaccttgt ctcaaaagaa aattaaaaat aaataaataa |
| 64201 | ataataaaaa gcaataaaac cagatacgac ttcactctct tggagacttt gggtttgatg |
| 64261 | agatataaag accgcaggaa ggacacctcg ttggtgagat gtggggagac ttagctgaac |
| 64321 | tcagatccag gaagggttgt gagggctcag gaccattcta aaggttatgg cactgaaaga |
| 64381 | agcctggagg taaaaggacc gcggcgtacc catgctcttt gtttcttcca tttagaatgc |
| 64441 | acgtcacgtg taaactcttt cctgattatt gccatctcca aggcaagaaa ccacagtcac |
| 64501 | ccccctgtga atgtgcagtt tccccaccag agtgagcaac gcttggcact tggcagagat |
| 64561 | gttcttggtg gatcaagggg tcttgagtct ctttattacc atctcttcct ggcaccaaga |
| 64621 | cttaatacat tgtcacccaa catctctcca gttttcacct ccatttcatg cccctgccac |
| 64681 | tgacctgagg tccccagcct caactgcatg gttgtaccta ctttctacct ggtctccctg |
| 64741 | ccctcatatt gcctcctcca ctccaaactc catgccacca ctggagcaaa cttctgaaaa |
| 64801 | tgcaaatctg gtcatgtcac atctctgcca cctggaaaag tggggagtgg gtaatggatg |
| 64861 | ttgcagaagg acagtactaa agggcgcacc aactctcatc tctggctccc agcagatgtt |
| 64921 | cctgaagctg gatcaggtcc ctgggaagca ggtcacaaag cattattact ctcgggtttt |
| 64981 | cccaggacag gggtatagat ttaccactca gcattattcc ttgtgatcgc tttcatgaag |
| 65041 | ccactgggga gtatgtccct tatcttgaaa ggagatggtg gaatagtcag ctggtcagtt |
| 65101 | ggtcccagct gtggctccgt ttgcctcttc atcttccacc ctggaaaaat tgagtcctct |
| 65161 | gctgatgatt ggagagaagg atggccaagc ccctccccca gaaccctgga gaatgaccag |
| 65221 | ccatttttta ttttttattt caagtgcatc atttcccctt cctagagccc caggaggggt |
| 65281 | gggaggcagc aggaatggca ttccccttcc cccaacacca tgcgcctggt ggtctttctc |
| 65341 | cagcgccctc agcttcctct cggggctgcc cttcaaccag caatgagcac cctctggagc |
| 65401 | ttctcttccc tccgttgctc cttcaagcct ctgcccacct tcccctccac cctccgcctc |
| 65461 | tactgccaga gcttctgccc aggttgtcac cttcactggc tctgctacat ggaggaaggg |
| 65521 | agaaacgtat gaggcctgaa aagggggaca gggagaagta gcctgtctta tggccatgcc |
| 65581 | tcgagatgga aaacataaaa accaaccact ctcttggcag tgggaagagg acttgctgct |
| 65641 | ggcttatctt cggtagatgc caaaactatg tctgtccgct tcccagtcct gagttataga |
| 65701 | aagccttttt tattgttggt gggggcatta gttgtggaaa aattcatggg gtttttaaaa |
| 65761 | atctttggca aaaaagggtt cgtggaagtt aaaactctac aaactattgt gcaaagaagg |
| 65821 | acttctagtg tccgtttgct ctcttcatag taaacttctc agagctaagt tagttagtcc |
| 65881 | tatttccttt ttagctccct gaaggagcag tgaatattga acttctgtga agctccagat |
| 65941 | tctaagcttt tacttccgtc cctttgtcac ttcacgttcc aaacggaaat gtttgcttcc |
| 66001 | tagttcaagt gtcacttcat ttgtaaatat cccctttcct ggcaatgttc tcattaacat |
| 66061 | tggaatttct atttccttaa catgattact tctgatgtgt agatttttaa tcaccctcag |
| 66121 | gttatttttc agactttgat ggacactgga tgatcatttt agtggttatt ctttttcatg |
| 66181 | gaaagggaag ataattctct caagtaaagt cgtatttaga agttgcatca aagcacaact |
| 66241 | aaaatatgaa atctccaaga tgacaggcat gaaaagcaac cttcggagtc gcattagaag |
| 66301 | aatgtgccgg gctcagtggc tcacgcctgt aattccagca ctttgggaag ccaaggcagg |
| 66361 | tggatcacga gggagactga gaccatcccg gccaacatgg tgaaacctcg tctctactaa |
| 66421 | aaatacaaaa attagccgtg cggtggcagg cgcctataat cccagctact cgggaggctg |
| 66481 | agtctggaga atagcttgaa cccgggaggt ggaggttgca gtgagcctag atcgtgccac |
| 66541 | tgcactccag ccggggcaac agagtgagac tctgtctcaa aaaaaaaaaa aaaaaaaaaa |
| 66601 | aaaaaaaaag aagaatgtac agaggtccat gcttattcta gctggggaaa ggcttttctt |
| 66661 | gttaaaacac acgtttgaga gaggcaagtg aatcaatcat tctaagtatc atgcacaagc |
| 66721 | tccctacttg agagatcaac cgcttagctg aagggagatt ctgttggggt cactcacatt |
| 66781 | ttgtagagct ttctgctgtt taggatgcat tttgacacac atgatctcat ttaacctgca |
| 66841 | tacaacctta caggtagctg gtgtctctgc ttgtaactga aaaacacaag gctatgtggt |
| 66901 | catttgccct cacacctggc aacatgtcag gtctggggtt caatcccaag ttcagcaacc |
| 66961 | cccagtacat gcctctgtct atcttaacat ggccaccctg acaggaactg acacctccaa |
| 67021 | atccaaatga gacctgatgg cttaaaatga gggttcggga aaagtagtag tgatgtcaaa |
| 67081 | aattactggt gctctggctc tctctctctc tgtctctctc tctgtcctta tatcttttta |
| 67141 | gcttaaaaag ccaataattg gctggtcaca gtggctcgtg cctgcattcc cagcactttg |
| 67201 | ggaggccaag gcaggcagat cacctgaggt caggagttca agaccagcct ggccaacatg |
| 67261 | gtaaaaccca atctctacta aaaaaaaaat acaaaaatta gccgggcggg gtggcatatg |
| 67321 | cttgtaatcc cagctacttg ggaggctggg gcagaagaat cgcttgaacc caggaggtgg |
| 67381 | aggctgcagt gagctgagat catgccactg cactccaggc ctgagtgaca gagagagact |
| 67441 | cagtgtgaaa aaaaaaaaaa agccattaat catttgtggt aagctccaca aggaagaatt |
| 67501 | tgttgcaatc ccacctgtgc ctggctccag caggtttcca ggaaacattt gttgcatgaa |
| 67561 | tgaacaactg aaatagggct tcccaggata taagtaagat tgcagagctg gacgccacat |
| 67621 | aaaggacaca gcagagtgca cacaacatac aatcagtagg ctgaaaaagg cggctcaggc |
| 67681 | aggcaccaaa gccgcactgg ccatacaaac aaagaaattc agcaaacaat gtggaaaggg |
| 67741 | gggggagcca aattccagaa atgcaaagac gtatcagagg cctgcaggct tacatgagaa |
| 67801 | ttcctggagc aggaaattaa atgcaagtta atgaagactt tgaaagcttt attcctgagt |
| 67861 | gcatgaaacc tgtgacatgg gtttggcagc tctttctgaa gcagagccat cgacgctggg |
| 67921 | gaagaacgca gggcacttgg catcacctag gagctttaaa aatacagatt ccagcctggg |
| 67981 | cacggtggct catgcctgta attccagcac tttgggaggc cgaggcagac agatcacttg |
| 68041 | aggccgggag tttgagaaca gcctggccaa catggcaaaa ccccatctgt ataaaaaaga |
| 68101 | caaaaattag cccggtgtgg tagtgcatgc ctgtaatccc agctactcag gaggctgagg |
| 68161 | cagaagaatc acttgaaccc aggaggcaga gggttgcagt gagccgagat catgccactg |
| 68221 | cactccagcc tgggcaacag agtaagactc catcaaaaaa ataaaaatac agatgcctga |
| 68281 | gctgcatgtc caggattgga ttggattggt ctggaggtgg cctgggcatc agtgttttta |
| 68341 | atgcctctcc ctcccttcgg cccagcaagt taagtctagt gaacactgac ttagaggtta |
| 68401 | gccaactcat actcctctat aaatgaagac acttgaggca tagaaggaga aagagccagg |
| 68461 | atcacccagc agatgagagg cagtggtcag aatggagccc aggaggccag tgctggctcc |
| 68521 | cgggccctcc ctctgaccct gctgccccag cccctggacc cctgcgggac caggctccta |
| 68581 | ggctcaggca gctttccttc tggctccccc ttctcctgcc tgcaggtcag catcacggtg |
| 68641 | atcgaggccc ggcagctggt gggcttgaac atggaccctg tggtgtgcgt ggaggtgggt |
| 68701 | gacgacaaga agtacacatc catgaaggag tccactaact gcccctatta caacgaggtc |
| 68761 | agtggccctg tggggaaagg agcctctgta aatatggcag gagggtggcc ccagccctcc |
| 68821 | ccaggagaaa taggagtggc tggaagtcaa ggagcacaca gggctaccag caggggtgat |
| 68881 | ccaccctgtt acaaccccca ggacactgac aaacagagcc acatcttctg aatcctctga |
| 68941 | accccccagt ctagagcaca ggccgacact ggtctttgaa gcgtacacca cccgggtgct |
| 69001 | cctgggcaga ccccagcagt gcctgtgaaa gaaccccccc ccaccccact ccccaagagg |
| 69061 | cctggttccc actgtttccc aggccagagc ccaaggacag ggctgcctca ttccaaccca |
| 69121 | tggtggcact ggcaccatta gtaagcactt tctaccagcc aggcatgctc ctaaatgctt |
| 69181 | ctgaactgga ccatccaaag ttgtcactat caattccctt ttacacaagg ggacagtgag |
| 69241 | acttctgaag gcagaccttg tccaacctca cagtgccagc atcgcccttg gcgtgctagt |
| 69301 | ggggttctgc cctcttggct cggctccttc atggttgcct aaacttctgc gggcttctct |
| 69361 | ccagagcact ggccttgatt ttagggtgtg aaagagaaaa catttggtac ccactcctat |
| 69421 | tctttccagc catttgaggc cacatcaagg gtgagggacc tcagagacct tctactttaa |
| 69481 | tctccccatc ttacagagga ggatactgag gcctccatat ggaagggcct tgcccacaat |
| 69541 | ctcagagagt gttgctgata gagcaggggc caggctgccc acccagcttc cagcccaggg |
| 69601 | ctctggccac catcacaaat atggcacctt ggttcccagg ctccagatgg gaccccatca |
| 69661 | taatcagctc tggggaggga tgtgagaaat agagaaatac actcactcat caaaacccaa |
| 69721 | agaatgactt agaggcatga agaacagcga aagtgagact tttaataacg gtcttgcaag |
| 69781 | atggagtgtc tggtaggcag gcacccctgg ggcagtcaca acaggaattt atctcctagt |
| 69841 | gtgcaagtcc ctcccccact tccccattgg ttgagcacta tggggttaca atcttccctg |
| 69901 | acgtcgccta agtttcattt tcccccttac aaggttatac cccgctcccc ttcgatttcc |
| 69961 | ccacttaagt ttcgatttcc caataacaaa actttcttcc ctttcatggg ctgacccctc |
| 70021 | ctctacattc tgttcgctta ttgtgtcctt ctaggtgcat gagccgtgtg gtttgttgca |
| 70081 | ttcgcaggtt ggatgccagt acttagactt atcctgcctt gaaaatggac cattgaaaat |
| 70141 | gttttctcac caggatgccc aggcctttcc cagatgcctg atcacacatc agactaaccc |
| 70201 | agaagccaca ggcctgggcc ctgccatccc tttcctggag ggttccccag tctgtttctc |
| 70261 | ctggtggctc caggcagtga ggggtgccag gctgtcccaa gtaaggcagc tgagtcccgt |
| 70321 | accctgaggt gggtgagggc tgaggtgccc cctgtagctt gggggagggc atttctcagg |
| 70381 | cagctctgag agtcctcaga ctgtgcacct gcaggaagaa aacccaggcc cacctctgcc |
| 70441 | cttcagccca ggcctggccg aactctcctt tgtgaccaca aattgactct gctctcccag |
| 70501 | ctgctttccc acaggccccc cgagctggac aacctgctca ggacccctcc agccccaagg |
| 70561 | acattcagga ttgacatctc tggccttacg ttaggacaca tcctgactgg acaagtctcc |
| 70621 | gcctagataa gcatctatgt ccccaagaca ggcatggaga caggtccctg tggccccggt |
| 70681 | ggttacttga tagcagtgaa gccacttctg ctaacttaag ggatctccct ccaccaggcc |
| 70741 | cacacacctt ggctttcatt caaattactt cctctttccc aagggcacag agctgctctc |
| 70801 | cctactctgt ggctccccca gaaaactcct actcacccgg cagggcccag ctcaaatgtt |
| 70861 | cttgctactg agcagccttg cctgctctgt ctgggtcaga ggtcagcact cccctttctc |
| 70921 | tgcagtgtta atgtcctgga gttgaggttg gtcctccgtg tctctgtcct gctggtggac |
| 70981 | tggaggctcc cagtctcctg cctcgctatt tgttcagagc caggcatgta ctcggagggg |
| 71041 | ttcagtaaca ctcacccaaa ggaagacaca aacatccact gagcatctgc cgcacaccag |
| 71101 | acagtggcac gtggggcaac atggaggctg ccgagatgaa ggaggcacag tttccgccct |
| 71161 | gcgcaggtcc cctccttgtt tcagcctaga atctttctaa attccctatt tacttcctct |
| 71221 | aagactgact attcattctg ctcatctttg gacaatctta aatgtgtttc tgatcacatt |
| 71281 | tttgttcttt ttcacaaacc caaatagctc tgattttgtt tctgtttcct ctaaactagg |
| 71341 | gtttctcaaa ctcagcacta tggtatcttg gactggataa ttgtggtggg atcgtcctgt |
| 71401 | gctgtgtagg atgtgagcag catccctggc ctctacccag tagatgccaa tagcaactcc |
| 71461 | ccttagttac agctaccaag tttccagaca tgccaaatgt cccctgaaat cgccctcagt |
| 71521 | tgagaccacc attctaaact atcttactat gattccttcc agccccaaat cagcctttcc |
| 71581 | agccccaaag ctttgtttcc cttttcttct ctttctatgc cacattcctt ttcaatgaag |
| 71641 | gacatcctcc tcgtcgtggg aatgtattat ttctgactaa ttatctgagc tggtaggaac |
| 71701 | cttcctttgg ctccattaaa acaacagagg gaggtgggaa caatcaacat gacttaggta |
| 71761 | tctaagctat tacaaaagga aaatatgctc aggaaataac aggaatttga ttgcagacag |
| 71821 | ctcagagagt tcgggattct ggctctagct cagctagctc gctcccctgg ggctggcaca |
| 71881 | tctgcgggca cctggcctga tcaccctgcc tggccctgag cctgcctgtg tgcagggaag |
| 71941 | ctcaaaacct ttgcccttcc ggaacaagct ggccattcac aaaccagact aaggctcctt |
| 72001 | tcctgtgtgt cctgtgtgtg catctggccc cgccctgggc atcttgggag cgccaccatc |
| 72061 | cttcctcctg ctgaaaatcc acctctcccc tttgccttgg atccttctct ttctacagct |
| 72121 | caaagaacct ggcattgcac agcccaactc tgtctttgtt tttcttccag ccccttcttc |
| 72181 | cttgcctata aggtgttcta ctccagcata tgctaagtac aaccatgaag acccatgtgt |
| 72241 | gctcaaggac aggaactagg agtctgtcac ctctctctgg gcctttcctc attcccacac |
| 72301 | acttctccag tgacctcctt tttccctcca cccctccaca gtcagaccac ttgggttgtc |
| 72361 | ctcacttcct ccccccagtc acccctcaat ctctgcaggg tttctgcctc cagatgtcat |
| 72421 | tgacactgct ctcctcaagg tcaccaagga cattgctgtt cctgagtata atgtccatgt |
| 72481 | catcctcctc tcccttgatc acccagtttc tttcatcccg ctgcctgctg ctcctcttgg |
| 72541 | gggcccctca cgccactctc ctgcttcccc tgtctctcca gctccatctc cccggcctaa |
| 72601 | cttcttggct gaaaggctgt gtcttcttgg agccctgacc tgggcctgct tcccctccca |
| 72661 | tgctctcccc aggtatctcc tccatctcca aggtgtcagt taccatcttg tgctgacgat |
| 72721 | tcccaaatcc atatccccac ccgcctcccc agagcttcag aactctggcc agcacaccct |
| 72781 | gggcctgtta gtgtcatgca cagctgtcgc tgcttgctat gtgcttaccg tggtgcacac |
| 72841 | ctgtgttaaa gacttgacca gggtgtctca ttgcaacttc acaaccacac cttgaagccc |
| 72901 | cccaagtgaa tggcacccct ggagcagttc agcacgacag ccctggatca tatctccaca |
| 72961 | ttcataggat aataggtaaa ctgaggctca ggcaagaaag tgagtgatct agcagtaaca |
| 73021 | gccaggtggg gctggctctg aagctggctt taccctcagt gctcaagggc cagcccccag |
| 73081 | cagccaggag cacgttctct tcggacattt accccaagca cattggaaat atttctccct |
| 73141 | tctcccccac cacagttcac caaagcagca gcccctctac tcactgcccc acaccctcat |
| 73201 | cctcagcacc tgcacaccca ggccagggga agagatgggg actgcaaatg tgcatggcag |
| 73261 | cccacagcac caaaggacag tgcacaccca tggcatttca catcgactgc caactaagtg |
| 73321 | cttggcacac catggttcat ctgctctgag gcttgcgtta ttttacagtt tcacctctct |
| 73381 | gaatttggga tgtgtctcct aggcagcctt gtggcttcat ggcaatgtcc atgtggctta |
| 73441 | ttaaaccgta gtatatctta cagccagtag ccactttaga tttggtaaaa tatgttacac |
| 73501 | tgtctcatca gagcctcata acaagactct gaggtgggtg tcattatccc catttcacag |
| 73561 | ctcaaaaaac tgaggttcag agaatgtgtt gctcaaggcc acacagttaa gaaggtgcag |
| 73621 | agcggggact agaacccagg tctgcttgct ccctggccct gtgcctcttc tgagcacagt |
| 73681 | acaagggcat gggctgtccc acctactgtt ccaagttcct ttcccacccc acactagcca |
| 73741 | gcaccagccc cacctagggg atggggctct gcttcctacc acccaccacc agggtcaatc |
| 73801 | aggatcagat gagttgcctc tgctgctcct ctgaactttg gggctcaggg caaatgtctg |
| 73861 | gggtgcagac aggccctgcg aggagggagg ggcccaacac tgcctgggct gttgctgctg |
| 73921 | ccctcagtga cagcaggcct ggatccttcc ccctccagta cttcgtcttc gacttccatg |
| 73981 | tctctccgga tgtcatgttt gacaagatca tcaagatttc ggtgagtggg gagcagcccc |
| 74041 | tggccggcag ggccactccc tcaaactgca cctttgtgca gacttgagag gggccccctc |
| 74101 | tgtctagacc catgataaag aggcagcgcc gtgcctggac acacggctgg gcaagtaggg |
| 74161 | acaggctgga ttctggcccc cacccacccc tttcattgga tgcctctgaa tagtgcattc |
| 74221 | gcccgtaaac agtggccagc aagaaactgg cgtgggcagg aggagctgat gctgagcctc |
| 74281 | agtgagccac cccactctgc caacgccctc ccacttcacc acaaagctcg ggtcccacca |
| 74341 | ctgccccatg gaaagcctca cccttgctgg gcctgacggg tgggctgtgg ttctgcctga |
| 74401 | aaggtgattc actccaagaa cctgctgcgc agtggcaccc tggtgggctc cttcaaaatg |
| 74461 | gacgtgggaa ccgtgtactc gcagccaggt gagtacctgc gcatggccag tcagtcgagg |
| 74521 | tgtgcatggc tcagggagcg tggtgcacgg ggcccttccc actgtggcag ttcctgggaa |
| 74581 | aaggctggca aggaccacag tctggcgact gttagtacac acagccatct ggcctgcgga |
| 74641 | gaagtggcgc gcacagtctg tacacaaatg cagtgagcaa ctgcgcaccc agtctggtgg |
| 74701 | gttctcactc ataattaggt gagtaactct gcacagagcc tggaaggagc ttcatcacac |
| 74761 | gctctggagt gcatggcagg ctggtgcatc actggccata ggcaggctga ccagtagtgg |
| 74821 | tagaggatgc ggtgagggag acccgtcaca gcctgcagag aggctgcgcc cagactcctg |
| 74881 | cctcctgggc tggcacctgc tgagcagaaa agaacccctt tccacccctc acaggaccct |
| 74941 | gccgtcccct tttacagttg aggctcagag ccagggcttg ccccagccac acagctggcc |
| 75001 | gggggtgaag tcaggctgtg tgtccccaga gcctcactga gccacactat ccaatctgaa |
| 75061 | aggggtgatg ctggtcaatg ccagatgtag tgcggcggcc tctgagggac tctggtctct |
| 75121 | gaaaacactt cttacaagag ccccaggctt ttgggcagcc atgtgtgctc cgtggcgtcc |
| 75181 | tgtagggtaa cagcaatggt gttggcaagg gaaccccaag gggtggccca gagggcttgt |
| 75241 | gaggtgggcc aggcagagga agtgcctcag cctgccagat gacagctcac cacgtgggca |
| 75301 | ggttagggac caccctgtgt ccctgaagtg aatgcactgc tcagagtgct gtaatgaccc |
| 75361 | cattaggcgc tttgacagtg agaacataat ttcacccaat tgtcacattg ggccctcaca |
| 75421 | aaacagtaat gtaggcagca gcatttcttc ccattttgca catgagataa atgaggctaa |
| 75481 | gagaaatgaa ggggcttaga gataccacta aactagtagg ttgaacaaat tggcgatcgc |
| 75541 | ttgtggttca acctaagaga cgtccagctg gtgcacatta caactatgac ttagaccagg |
| 75601 | gaatctgagt ccaaaaccaa gattatacac ataaaaatca caagatcata gaacactcaa |
| 75661 | gctgcccaag acctgaaaaa catctagtcc agcctctgtg aagataaact gaagcccaag |
| 75721 | gtgatgaagg ggtttgtcca ccatctagga gtagcacccc tgtctccagc tcccaggcca |
| 75781 | gtgcctttgc caccacccca cacatgccat agccctcagc ggagggcagc cctgctgacc |
| 75841 | atcagtgcca tggggagcag ggaagagtcg gcctcctgcc atgttgcttt ccaggaagca |
| 75901 | tgcatagccg tggactactt gcttggaatg actggcatcc ctgaggataa gctcctccca |
| 75961 | gggctgagca gtggccctgg taccactggc aggcctgagg ccgagagcca agagaaagtc |
| 76021 | agggggtgga gaaaaaggca gatattagtc taatatggac ctctgggctt ctgggtagta |
| 76081 | aagtcctggt gaggaaacca ccagggcatc agtaagtgcc agatgttggg ctaagctcac |
| 76141 | tgtcccagca ggcctcaccc tcacgacccc atataggcat cattcccatt tcaccataag |
| 76201 | gaaactgagg ctcagcaggc ttaaacagct ggtccagggc cacattgtga gtaagcggca |
| 76261 | gaaacaggat gtgggccgag gtctccactt gggtaaccta gctcgagatt tgctgcttct |
| 76321 | gctgaatcca gccctgagcc tggtgccctc ccagggacag gagagcctga agtcacatgg |
| 76381 | gtcctggagt tgagacccaa tgtctttact ctgatatctc cagtttggcc tgaacttctt |
| 76441 | gatgagtcgt tggttccagc cagggcattg ttggacagca tgctcccaga gacactcctg |
| 76501 | ggtgctgctg ggaagctttg ggtggtgtcg atatctcttg gaactggaag gggtttcccg |
| 76561 | agagaacctc ttctcaccct ggcaggacca cattttgtca tttgtaccac cagatgcaac |
| 76621 | atcaggaaac tgtttactgt ttgctactct ctgttggggg tgaggacggg ccatacactc |
| 76681 | atcctcatag atcatagagg aagtcagggg catacactca tcctcataga tcataaagga |
| 76741 | ggtctgggat gcagcaactt ccccagctgg gctacccttg actggcaggt gtcagcccca |
| 76801 | ggctgaaggg aggtgaaatg atgtttcagg ctggtaagca tatagctcag gatctcatgt |
| 76861 | ggtccttgta tctctagcct gagatcacac agtattagga ctagaagggc ctcagaagtc |
| 76921 | atgagtcaag tgttaacctg atgtagggac ccccacagca ccatctctga ttggtcctct |
| 76981 | aaccttgctt agtcctccca gaaatggggc actcactact tcttatttta gatatcttca |
| 77041 | ctttctttag gtcatctcaa aatctgtcac cctgtaacct tcacccactt tcacacgtct |
| 77101 | ctctttcagg acaaaaagaa aaatagcctt taaatttgaa ggtaaatttt acattcctcc |
| 77161 | tgaaccgtat tctctttctt tgctaaatgt ccccaattca tctaaccatg cacacctcta |
| 77221 | tacatctacc cctgtatcca tccatccact cacccaccca tctacaaatc catccaccca |
| 77281 | cctgtccaca gatccaccca cccatccatg tatccatcca ttcacgcacc aatccatata |
| 77341 | tccattggtc cacccaccca cccatccgtg aatccatcca cccacccacc cgcctgtcta |
| 77401 | cagatccacc cactcaccca tatatccatc catccaccca cccacccatc catatatcta |
| 77461 | cccacccatc tatccacaga tccatccacc tacccaccca tccacagatc cacccaccca |
| 77521 | tatatccacc catccaccca cccatccata tatctaccca cccacctgtc cacagattaa |
| 77581 | tggctccttc tatccagtaa tccattcacc atctgtccgt ctatctctca gccggccagg |
| 77641 | cagctcacca cccatccctc tgcccattta tgcacccatt cattgagctt tctgtccatc |
| 77701 | tcctcatccg acacacatct gctctggcaa gacctgaagc tcctgggatt cctgggacat |
| 77761 | ggcccctccc tggaggcctg ccagcctggc tgaggttctc cagtgactcc tcatccacgt |
| 77821 | gccttcctgc cctctcacct tgcttgtccc tctcttcaga ctctactgtg gcattatctt |
| 77881 | ttctgagaca caggtggcct ggctattccc agaaaggcca acctctgtac acagccccta |
| 77941 | acatctccgg gagcccacca gggccacagg cttgcaggac acaggcctgg gctacggtag |
| 78001 | aaggtggaag aggagggagt gaaggaggga ggtgctgagt ccctgagagt tcagtgccca |
| 78061 | cctaacatcc cgggaagcag attcctaagg cacagtgatg cgcttccctt tgcttgccca |
| 78121 | ctgggcttaa agtaaacctg aaggggcaaa tccttcctaa agcgcctcct tccctgacat |
| 78181 | ccgtcctttc ctccacacag tcataggtca tgggacactg ggatgagact gacaatccct |
| 78241 | gtctttagga gactctcagt ctaaaatcag agccaaacaa tacccagatc agcacagtga |
| 78301 | gatgggaggt gtgcatggag agcacagaga acaaacacta aagagcccag gatcaactct |
| 78361 | tccgaggaag tcagggaggg ctgcatggag gaggagagac ttgagcaaaa tgagtagaag |
| 78421 | tttacaaatt gaatagggag aaagacaggt ggaggtggag agaacaggaa tgagggcatt |
| 78481 | ccaagctcaa agagcagcag atgcaaaggc atggaggtat aaaagggcat ataaaaccgc |
| 78541 | aacacctgtt cagcaaggag gaacacaggg ctgggtgggc tcagcagagt gagtcaggga |
| 78601 | gcactaaatc aggagcctgc aggccatgcc ttttgtcttc accccaaggc actgggaagc |
| 78661 | cccagagggg ctgaagatgg ggagcaggag catgagcctc cacctgggaa ggccctgtgt |
| 78721 | gcagctgtgt ccttcacagc tatgtgaagg ctggctgact ggggtggggc catagaagag |
| 78781 | gccacaggca gaaaaaccca attaggagga ggctgtcgtg cttccaagtc acaccactca |
| 78841 | tgggatatat gtccatgggc aggccatgca cccactgccc cagctccaga ggacctcaga |
| 78901 | ctctatcaca ccctgggcta gggacaggca gagctgtggc cccaggtccc gggactctag |
| 78961 | ggaccaagac agcatttggt tctgccaccc cttggcctgc cctgagcact gggagacgct |
| 79021 | ctgggtgggg gtgtctggtg cagtgcccac ccctcatcat tgcagagcac cagttccatc |
| 79081 | acaagtgggc catcctgtct gaccccgatg acatctcctc ggggctgaag ggctacgtga |
| 79141 | agtgtgacgt tgccgtggtg ggcaaagggg acaacatcaa gacgccccac aaggccaatg |
| 79201 | agaccgacga agatgacatt gaggggtgag gcccagctac cccagcccct ggagtctgag |
| 79261 | ccagcccttc cttcccccag ggagcccccg gtgagggttt gctgagagca cctgccaccc |
| 79321 | gtctccctca ccctgctctc gctcactctc ccaagcagcc ccactcttcc gaggcagggc |
| 79381 | aggggcaggg ggtgatggag gatggctgag aatatcagtc ctttgacatc ctttctgcca |
| 79441 | gtgagaaatg ggttgagaag agtcgatgct ggcagaccag aaccagcagc tgctgggtcc |
| 79501 | tgagtatagc agcaggaagt gctgggagag ccaaggtttc ttctggtgtg ccaattccag |
| 79561 | gcagctgata gcagctgttg agaaatattc tgtgaccttg tctggagtca gtgggtaaaa |
| 79621 | gtgctgggaa tcaatgaatc ctgtcttttg tgagctgggg gagagagagt ggctactgtg |
| 79681 | aagtttttgt ctacgaaaca aaagatacgg cacaaaacta taaaacactg acaataatac |
| 79741 | taagatcata tcccaatgta caatctacaa agcactcata tgttttagcg tgcttgagcc |
| 79801 | gatcatgttc ttgggaggtg ggtattatta cagccacctc aacagtgagg aaacggcagc |
| 79861 | tcagtgagat gggaagattt gcccagaatc ccacaagggg caagttccgt gaacacaagt |
| 79921 | gtctaatcct agccttgctc cctcagctgt cccttagaga ctgtgccagg acccaggagt |
| 79981 | gtgtagatgc caggatgggg gttggggacc tggacctggt gaccatggcc ctggctgtat |
| 80041 | gtgctggcag gaacttgctg ctccccgagg gggtgccccc cgaacgccag tgggcccggt |
| 80101 | tctatgtgaa aatttaccga gcagaggggc tgccccgtat gaacacaagc ctcatggcca |
| 80161 | atgtaaagaa ggctttcatc ggtgaaaaca aggacctcgt ggacccctac gtgcaagtct |
| 80221 | tctttgctgg ccagaaggta ctggggtatg aggtacagga ggctggggct gggtgacagg |
| 80281 | tatcacaagg gcaggctgat gggtgtgggg cctggggcag ctgggcaggt aagacaaggc |
| 80341 | tggaaatcat gttggccagg gacggactta gaggatcctc actgcctcct ggagtcctta |
| 80401 | gactgagagc tgcgcagggg gcatgagagg gaggagaggc tgggggtgtg ggaagagtgg |
| 80461 | agacaagtcc acacacaggc acgcacacac acatgcaccc actcatacac gcacgcatac |
| 80521 | caactcacac acgcacgcat accaactcac acatgcagac acgcacccac tcacccgtgc |
| 80581 | acacgtgcac ccacgaatac acacacacat gcacccattc acacacgcac acacacccac |
| 80641 | tcacacatgc acacatgcac ccactcacac acgcacacac acccattcac acatgcacac |
| 80701 | atgcacccac tcacacacgc acgcataccc actcacacgt gcacacatgc acccacgaat |
| 80761 | acatacatgc acccactcac acgcacacat gcacccatta acacacgcac acatgcaacc |
| 80821 | actcacacac gcacacatgc atccactcac gcacacatgc acccactcac gcacacatgc |
| 80881 | acccactcat acacacacat gcatccactc acacacgcac gcgcacccac tcacacatgc |
| 80941 | acccactcac acacgcacac atgcatccac tcacacacgc atgcacaccc actcacacat |
| 81001 | gcacacatgt gcccactcac gcacacatgc gcccactcac acacacacac atgcacccac |
| 81061 | tcacacacac atgccacgcc ctcacctgtg ctcccccttc caccagggca agacttcagt |
| 81121 | gcagaagagc agctatgagc ccctgtggaa tgagcaggtc gtctttacag acctcttccc |
| 81181 | cccactctgc aaacgcatga aggtgcagat ccgagactcg gacaaggtca acgacgtggc |
| 81241 | catcggcacc cacttcattg acctgcgcaa gatttctaat gacggagaca aaggtcagca |
| 81301 | gcgggagacc cggtgtgctg gggggcagag ggagtggcct gaagggaagg aatatgtgag |
| 81361 | atgccctctc ttcacaccac cgtcatcagc tctggtcctc agcctcacag gtgcttccag |
| 81421 | ggccttgcca gcagcccctg cctcaggggg agggtgagga tttgtggctc aggtcggctc |
| 81481 | agagctggcc tgagcccagc tgactggatt tgtcagctag tagttgtgtg acctgttttc |
| 81541 | ccatttgtaa agtgaaaata ataatgatta tatgatatga taattatagt atgatatgca |
| 81601 | cattatagca gtgtatatac tgcatttgta tataattaca gtgtgtgtgt atatatatat |
| 81661 | gatacctaat agtagtgcct atctgcagaa tcgcagggat tgactacctc ctaggatccc |
| 81721 | tgccctgtgc tgggttccaa ggacatagag tgagataaga atctattcct ggatcaaagt |
| 81781 | gctcactgtg tattgaggag atagatgtgc taccaaatgg tcaaaacata aggcagaaga |
| 81841 | cacaaaaggc cacatgttgt ctgatccatg tatgtgaaac atccggcaaa tccacagaga |
| 81901 | cagaaagtag attgccggtt tccaggtgct aaagggaggg gattgggact aactgctaaa |
| 81961 | taggtgcaga gtttcttttg agggggtgga actgctctgg aagtagatga tggtttatag |
| 82021 | cacagtgaat atacgaaaaa ccactgaaat gtacacttta aaagggtgaa ttttatgtca |
| 82081 | tgtgaagtat atctaaattt ttaaaatgct ataagtgtgt tttttaaaaa aaggcagaat |
| 82141 | aaaaaggaaa tgaaccaaaa aaactggatc atgagacccc tgtcagaggc ctgacaaatc |
| 82201 | ttagccataa gagaagggac ttgtgcctag gaattgggaa ggcttcgtga ggaagcagca |
| 82261 | tttcacaggc agagatggag agggatggcc ttcagcaagg aggggagggg tctgcactgg |
| 82321 | cagggtcacc gtggtgggac agagctgcaa aaatggcaca gggcccaagg tcatgctgcg |
| 82381 | gacttggaag gactggctct gtcctacaag tagactgttg acaattcctg ctctaagttc |
| 82441 | caggaagcag ggagctagac tttgccttcc cactgcttcc agctcaggcc cccgcaggga |
| 82501 | agaaatagac ccaagagagt gctccacccc agcgcagtgt aagaaggctg ccccacacct |
| 82561 | gaatggcaca catgaagttc tcagctcagc acccaggagc tggaggccag ggacccccaa |
| 82621 | aagaggcccc tcttccccag ggaaactgga tgtgacgctg ccccctcatt tttgttccca |
| 82681 | ctgccacagg cttcctgccc acactgggcc cagcctgggt gaacatgtac ggctccacac |
| 82741 | gtaactacac gctgctggat gagcatcagg acctgaacga gggcctgggg gagggtgtgt |
| 82801 | ccttccgggc ccggctcctg ctgggcctgg ctgtggagat cgtagacacc tccaaccctg |
| 82861 | agctcaccag ctccacagag gtgcaggtgg agcaggccac gcccatctcg gaggtgagac |
| 82921 | cccaggcact gtgctgtccc ccagggcctc cagctcagcc cccttctccc tgagtcacct |
| 82981 | gggtcccagg ctttgcctgt tgctgccctg tctacctgag gagtcatacc tcaggataag |
| 83041 | gcttctcccc aggaagcaga ggcccccact cagccccgca acaggactct gccctcgtcc |
| 83101 | actgcctgcc tgtgctctgt ctcacaacgg ccgccaggtc tcacgatggt gcctcccctc |
| 83161 | tgcccctctt cccagccacg gctgtccctg tggtgtctgc agccagccct gcttccatgg |
| 83221 | atatccaggc tggggggccg ttggggtgtg ggctgcctgg ccccaaagcc gggtgggatg |
| 83281 | cccccaccat ccacccccac ggcctgtctg tgagacggaa ggaggcctgg cccccgacct |
| 83341 | gccaccctta ctcaagctca ggaacgcttt gaactgcctc cccacagagc tgtgcaggta |
| 83401 | aaatggaaga attctttctc tttggagcct tcctggaggc ctcaatgatc gaccggagaa |
| 83461 | acggagacaa gcccatcacc tttgaggtca ccataggtga gtgctgggcc cacgggggct |
| 83521 | tcgggcctag gtgagtgctg ggcccacggg ggcttcaggc ctaggctgca ggtggtaagt |
| 83581 | gtgaggctct gcacctcagc cctgtcctca ctcttgtagg gaacgttgac ccctttcccc |
| 83641 | tgagaggcct caagcaccca caaaacacct ttgtgtgtgc atggggtgga ccacgcccca |
| 83701 | taaaggggcc aaccctcagc tccacctaca gggtgaggct gtgtttggga cctggggcag |
| 83761 | agggcaggga gaagcccttc ccggggctgg agaggagaga gtgggaactc aggccacagc |
| 83821 | ctgggccagg gctgggcctc gcactcaggc gtcatgagga ctccctgact ctcagcggag |
| 83881 | tggggacacg ggctaatgct ctctggctaa tgatgtgtca cgtctgagcc ccattccctg |
| 83941 | actctccctc tcccctttcc caaacccaag gcagcaccgg attggacggt gggaggggcc |
| 84001 | tggtggggaa tgcactctag gccttctggg cccattctgc tccaggcaac tatgggaacg |
| 84061 | aagttgatgg cctgtcccgg ccccagcggc ctcggccccg gaaggagccg ggggatgagg |
| 84121 | aagaagtaga cctgattcag aacgcaagtg atgacgaggc cggtgatgcc ggggacctgg |
| 84181 | cctcagtctc ctccactcca ccaatgcggc cccaggtcac cgacaggtgg gcccagcctc |
| 84241 | ccatcctcct gtggcctggc cctcccacct ccctgggccc ccaagacctc acctcccgcc |
| 84301 | tctgcccacc ccaggaacta cttccatctg ccctacctgg agcgaaagcc ctgcatctac |
| 84361 | atcaagagct ggtggccgga ccagcgccgc cgcctctaca atgccaacat catggaccac |
| 84421 | attgccgaca agctggtcag ggccaggcca ggggccagca ggggtggggt cctgggggag |
| 84481 | acggcgctgt cagagggcct tccctggaac acacaaagct ggtctgccca gggggctgca |
| 84541 | ggaacggccc cagcaggacc tctgttccca ttctcagccc caggtcccag ggcacagaca |
| 84601 | aggacaggag ccacctctag tcactcagcc tccagtgaat cctggggcag gagtgttggg |
| 84661 | ttccctgatt tttccagaaa tcttaaaagc aaggctaggg agacaggtgt ggtggctcac |
| 84721 | gcctgtaatc ccaacacttt gggaggccaa ggcaggtgga tcacctgagg tcaggagttc |
| 84781 | gagaccatcc tggccaacat agtgaaatgc cgtctctact aaaaatgcaa aaattagcgg |
| 84841 | ggcttggtgg cgggcacctg taatcccagc tattcgggag gctgaagcag gagaatcgct |
| 84901 | tgaacccgtg aggcggagtt tgcagtgagc caagatggcg ccactgcact ccagcctggg |
| 84961 | caaccgagca aaacttcatc tcaaaaaata aaataaaata aaataaaaca aaataaaaaa |
| 85021 | gcaaggctag ggaagggaaa tggggaaaag ggagaagcga gcagtgtctg aagctcagga |
| 85081 | gctgccccct cctcctctgc ccacccctct ccccccatga gccatgggca ctgactgggc |
| 85141 | gaggagtgca gctgttggca tgagacctcc ttctccccag cctggggaag cagggaccag |
| 85201 | cagataatcc cgcccttccc tgagctgttg ctgttccctg ttgaagttcc ctgaagctca |
| 85261 | gccagctcat attttataaa aatgaattaa aacctccaaa aaaatcaagg ctaatatgag |
| 85321 | gtctgctccc agccccctgc caatgtcctc cttgtcatcc ctgtcttgtg actataacca |
| 85381 | caattaaagc ccatagcctt tgcagtgtga cctgggtctg cagttatctg caaggcttcc |
| 85441 | ccctaaggag ctggggtaca agctgacctg tgtttcctgt gggacctggg tctcacagcc |
| 85501 | ctcccagtgc tctcagagcc agagccactc cctcttcccc caggcatctg ttcccatccc |
| 85561 | ctgccacagg ctccagcatt ctggagtgac gtcaggatct tggcacatcc agtcctaggg |
| 85621 | tttccctctg tctctggcca gccctgagcc ccgtagccgt gagttctgcc caggccctgt |
| 85681 | gagctcacca gagccacaga ctcacagccc agaggtggct tcttccttca ggaactgaag |
| 85741 | aacccccatg aacaccaaca tctccaggtt ctgagaacag aacctgggaa attgatgact |
| 85801 | tcctcatgat gaccgatact caggatggcc ctagcgagag ctcccagatc atgaggtccc |
| 85861 | tcactcccct gatcaacagg gaggaggcat ttggggaggc tggggaggcg gggctgtggc |
| 85921 | ccagcatcac ccacactcct gattcacagg aagaaggcct gaacgacata caggagatga |
| 85981 | tcaaaacgga gaagtcctac cctgagcgtc gcctgcgggg cgtcctggag gagctgagct |
| 86041 | gtggctgctg gtgagaaggg aggggcggca ggtgggggga ggatgaggcg gaactggaca |
| 86101 | aaggggaccc tgccagggct gggcagatga ggaagaatct acagagcccc tgctggtcag |
| 86161 | gctcgctaaa ccccggctcg ccctaccccc agccgcttcc tctccctcgc tgacaaggac |
| 86221 | cagggccact catcccgcac caggcttgac cgggagcgcc tcaagtcctg catgagggag |
| 86281 | ctggtgagga cgcaactgga cggcgggggc tggagggagg gcctggttgt gagaaggtgt |
| 86341 | cacaaccccc tttgtcatgg cccgagctgt cccccagttg ctgggttcac tgacaccccc |
| 86401 | tccttcgcag gaaaacatgg ggcagcaggc caggatgctg cgggcccagg tgaagcggca |
| 86461 | cacggtgcgg gacaagctga ggctgtgcca gaacttcctg cagaagctgc gcttcctggc |
| 86521 | ggacgaggtg cggcccaagg ggtcggggct ttgcctcatc cagggctggc tctctggggc |
| 86581 | ccctcaggat cagggggctc aatcaggagg ctagaccccc ttacccactg cccctggcca |
| 86641 | ccccccaacc cccagcccca gcacagcatt cccgacatct tcatctggat gatgagcaac |
| 86701 | aacaagcgtg tcgcctatgc ccgtgtgccc tccaaggacc tgctcttctc catcgtggag |
| 86761 | gaggagactg gcaaggactg cgccaaggtc aagacgctct tccttaaggt gctggagggg |
| 86821 | gcaggatgga tgggggcctg ggtcctttca ggggagcagc agcccccacc tggggctgga |
| 86881 | agcctgagtg acagggtggg gccttcaagc agcaggtgct cagagaagag ccaagaatgg |
| 86941 | gaaggacgtg ggggaagggg tggggaagga ggtgggggaa ggggtggggg aaggggtggg |
| 87001 | gaaggaggtg ggggaagggg tgggggaagg ggtgggggaa ggggtgggga aggaggtggg |
| 87061 | ggaaggggtg ggggaagggg tggggaagga ggtgggggaa ggggtgaggg aaggggtggg |
| 87121 | ggaaggggtg gggaaggagg tgggggaagg ggtggggaag gaggtggggg aaggggtggg |
| 87181 | gaaggaggtg ggggaagggg tggggaaggg ggcatgacgg ccccctgccc atcctcctgc |
| 87241 | ctccaccgga ccccagccca gcgggatggc tgtgaagggg cagggcagct ctgaccaggg |
| 87301 | cctctgccgg cccctgcccc ttccccacct cttgggccct ggcagccgtc aggctcctgg |
| 87361 | tgaccccatg cccaccccca gctgccaggg aagcggggct tcggctcggc aggctggaca |
| 87421 | gtgcaggcca aggtggagct gtacctgtgg ctgggcctca gcaaacagcg caaggagttc |
| 87481 | ctgtgcggcc tgccctgtgg cttccaggag gtcaaggcag cccagggcct gggcctgcat |
| 87541 | gccttcccac ccgtcagcct ggtctacacc agtgagtgag gacccctcac tcgaagcctc |
| 87601 | acctgggagg gggccggagg ggctgcccat cctggaacct caggctgccc ttccccacag |
| 87661 | agaagcaggc gttccagctc cgagcgcaca tgtaccaggc ccgcagcctc tttgccgccg |
| 87721 | acagcagcgg actctcagac ccctttgccc gcgtcttctt catcaatcag agtcagtgca |
| 87781 | cagaggtgag ggcctgggag gagggagtgg ctctggcttt gagagcacca gaagcctgtg |
| 87841 | agcctgtggg gactggagcc tgtggggcct gtgagcctgt ggggcctgga gccacaggtc |
| 87901 | agccagtgcc ccggtttctc aggggaagcc ctgcagataa ctgcagcaga cgcgggtcac |
| 87961 | actggaaagg ctacgggctt tagctgtggt tatattcaca agacagggac gacaaggagg |
| 88021 | acattggcag ggggcttggg ccaaacctca tattagcctt gattttaaga tttctggaag |
| 88081 | aatcaagaca cctgtcactc ctgcccccag gttccgtgga agctgagtga gtaggggtgg |
| 88141 | cttctgtttg tgcatctgcc cccccctctg tcacttgtcc cctgtctccc catcccatgc |
| 88201 | tgcaggtgct gaatgagacc ctgtgtccca cctgggacca gatgctggtg ttcgacaacc |
| 88261 | tggagctcta tggtgaagct catgagctga gggacgatcc gcccatcatt gtcattgaaa |
| 88321 | tctatgacca ggattccatg gtatgggtgg gctctggtgc cagggacccc agcagcacca |
| 88381 | cacctggccc ttgttccctg cactccacct gcgctgaggc tgtgccctca cctccgccac |
| 88441 | ccacctgcag agctccttgg ccaggtgctg gctgggaagc tgggagtggg ggctgacatc |
| 88501 | tagctcctcc tgcctgcttg cggcagcctg accccttccc aggccagcac atcctcacct |
| 88561 | cctgtctcct acagactgag ccctggaggt gtgccagcaa gtcctgctgt cactagcctc |
| 88621 | cccaccccag ggctccccga cccctgccct cacctaccac aaccagtaac agccttgcca |
| 88681 | tcctccgtgg aggttctgga gcagggagta agggattggg gtgacccccg agagttagcg |
| 88741 | agccaccttc tcacatacca cctcagctgc tgaggtgccc agatagccct ggactagagg |
| 88801 | taggaggacc tcatgtctag tcctgagccg ggcatctgcc atttactgac tgcttggcct |
| 88861 | tgaacaagtc tttggtgggc cctgagactt ggtttcctca tctgtaaaat ggggtgatca |
| 88921 | cacctgtccc ttacaacaac tcatggcgct gtggtaagga ccaaacgaga tcacaggtgt |
| 88981 | ggacagcaag caccaagagg cttctgggtt gtctgttgcg tcagggcaaa gctgacttca |
| 89041 | tgggccggac cttcgccaaa cccctggtga agatggcaga cgaggcgtac tgcccacccc |
| 89101 | gcttcccacc tcagctcgag tactaccaga tctaccgtgg caacgccaca gctggagacc |
| 89161 | tgctggcggc cttcgagctg ctgcaggtgg gactgcaggg aagaggggct gaggcctggg |
| 89221 | gactggaggc tgggagttgg ggcctagggc tgggggctgg accctgaagg ctggggcctg |
| 89281 | gggtctgggg cctgggggtg gggcctgggg gctggaggct ggggcctggg gctgggggct |
| 89341 | ggggctgagg gctggggcct ggggcctggg gggttagggc ctgggcgtta ggtcctgggg |
| 89401 | gttggggcct ggagctgggg cctgggggct ggggcctagg ggcctctctc ctacccatcc |
| 89461 | tgaccaaagc tgggggcgtg gtccttaggg ggtttccctg tctccccaga tcatggcaaa |
| 89521 | caactcatgg ggaaagagac cctgtccctg ggctccccag tgtgtgacct gtgaccccca |
| 89581 | ttccccagat tggaccagca gggaaggctg acctgccccc catcaatggc ccggtggacg |
| 89641 | tggaccgagg tcccatcatg cccgtgccca tgggcatccg gcccgtgctc agcaagtacc |
| 89701 | gagtggaggt gcgagggctc tgtgtggccc ttcccttttg gatgaggagt ctgaggaccc |
| 89761 | aggaggggaa gtggcttgtc atccccctcc caccaccatc accaggagcc agcagcagac |
| 89821 | ccaggctcct gccccagccc agggcctgcc cccatctgca ccaagtcgca ctcccatgtc |
| 89881 | ccaggaccat gcccagccct cctgtctgca cccctgaccc tgcacccctt ctccgggcag |
| 89941 | ccccaccctc atccctggag aacgcctgcc cctcctctgc tctcctggcc ctgctcccac |
| 90001 | cctttgaaat ctggggtgaa ctactgccca tcccactgcc cggttcccta ttggatggtc |
| 90061 | cagcaccccc atggctggct cccttgacta aggggctctc ttctctgcac ccaccctcca |
| 90121 | acctctccca ggtgctgttc tggggcctac gggacctaaa gcgggtgaac ctggcccagg |
| 90181 | tggaccggcc acgggtggac atcgagtgtg cagggaaggg ggtgcagtcg tccctgatcc |
| 90241 | acaattataa gaagaacccc aacttcaaca ccctcgtcaa gtggtttgaa gtggtgagtg |
| 90301 | caggccctgg cggaggacat cctgtggcca gtgtggctcc actcagccag ccggcttcct |
| 90361 | ctgccccttc ttggaggctc aggctcctgt cgccaacacc ccaggacctc ccagagaacg |
| 90421 | agctgctgca cccgcccttg aacatccgtg tggtggactg ccgggccttc ggtcgctaca |
| 90481 | cactggtggg ctcccatgcc gtcagctccc tgcgacgctt catctaccgg cccccagacc |
| 90541 | gctcggcccc cagctggaac accacgggta tggccacatc caccctgcca ccaagcctgg |
| 90601 | ctccacgccc cccagggaag ggctccaggt ctccgcccag tccaagccgc gaccagagtt |
| 90661 | tgctctttgg ggccgagggg gtgggcatag aagaaggtca ccctggtggc caaggaaggc |
| 90721 | tgcccaaggt gacgccagct ctacagccag agagggcaga actggagaca gcctgggggg |
| 90781 | gcctgagggc agaaagcccc ttgcgctggt tgatggagaa gacccaggcc tccccgccac |
| 90841 | ctccctccca ccggcctccg cagcttcccc ttatctcggg ctctcttctt ccctccccat |
| 90901 | gcccatattt tctcccttga ctgcctgtcc ccctctccct cagaaagttc cagaatcagc |
| 90961 | tttagttctc cgcaccttcc tgccccgcca atctcactgt tcctcttgct ccttcctctg |
| 91021 | cctgtctgtc catctatcca tctgtccagt caggcttctc cggcgctgcc gtgtgctgtg |
| 91081 | caatgggggc tcctcctctc actccacagg ggaggttgtg gtgactatgg agccagaggt |
| 91141 | acccatcaag aaactggaga ccatggtgaa gctggacgcg gtaaggcggg tggggtcagc |
| 91201 | cccctgctgg ctgggaacat gcaacttcgt gtgacacatc ctgtgaacca gactatctgc |
| 91261 | cccgagagaa aaggacacta agagcctcct ttggcatgtg actgtcgggg gctgagcttg |
| 91321 | attgtgtgtc caaacagctc cctcctaata ctatcctgga atgcacacgc gtgcgtgtgg |
| 91381 | tatgtaaatg tgcatgtgtg tgcgcatgtg tgtggtgtgc atatgtgcat gtgtgtgtgc |
| 91441 | atgcatgtga tgtatgtgca tttgtgcgtg catgcgtatg tgtggtgtgc atatgtgcat |
| 91501 | cgtgcatgta tgcatgtggt gtatgtgcat gtgtgtgtgc atgtatgtgg ggtatgtgca |
| 91561 | tatgcacgtg cgtgcgtgtg gtgtgcacat gtgcatgcat gtgtggtgta tgttcatatg |
| 91621 | cacgtgcatg catgcatgtg gtatgagcat gtgcatgtat gtgtgcatgc gtatggtgtg |
| 91681 | catatgtgca tgtggtgtgt gcatatgtgt gtgcatgtgg tgtgtaaatg catatgtgca |
| 91741 | tgtgtgcagt gtgcatgcat gcgggtggta ggtaaatgtg catgagtgtg gtatatatgc |
| 91801 | atatatgtgt gcatgtgcgc ttgtgtgcat atgtgcatgt gtgcatgtgg tatgtaaatg |
| 91861 | tgcatgtgtg catgtgtgca tgtggtgtat gtgcatatat gtgtgcatgc atgcttgcgg |
| 91921 | tgtgcttatg tgcacatgcg tgcatacatg tgggtgtgtg catgtgcact ggcatgtggt |
| 91981 | atgtgtgtat gagagacaga gtgagccatc acagtttatt ataatcacct gctataattt |
| 92041 | ttactccaac aaatgacacc ataggtaatg attccttatg agatcatcag tgtcctttcc |
| 92101 | aaagagtccc tggttttcca tcccagccat tcacaaatga cacttccctg gcctgggtca |
| 92161 | gaggaggatt tccaggtgga agctccttat tcctcctggc tttgggccta ggactgttca |
| 92221 | tctgccagag agagaactgc catctggggg ttgggtgtgg aagtggcgta caagctgtag |
| 92281 | accctacagg gaagggcaga acgtgggcca gcctggaaca cagcccactg ttcctttggc |
| 92341 | cacccaaaga aacatcctca ctcagaccct ttgcagactc accagtcaga gtgctcttat |
| 92401 | taatgcaaag ttggccttgc gggcacaggg gctgccagca cacagtaatt tccatctcgg |
| 92461 | ctcctccaca caccctgtgc taggccactg cctgccactc caggccccag tggctgattc |
| 92521 | tgcccctgtc tccttggttc ctctgcagac ttctgaagct gttgtcaagg tggatgtggt |
| 92581 | gagtgtgggg gccatgcagg ggctctgggg tgagagggtc ctttattggc aatgatgtca |
| 92641 | ggccaaaggg tccagcatca gggacagagc cagccccagc ccccgcatgc ttgggccagc |
| 92701 | acacctcccc taagatgcag tggaaaagag ctgggtgagt gcaggactcg gtgccagtcc |
| 92761 | tgcttgggcg agtggatcgc actggcaggt cacaacactc tgtggcctgt ggcaccttct |
| 92821 | ccataaaata aggccctgcc ccttggacat gggtggcccc acagagcctc atggagccca |
| 92881 | aggcttcctg ctgacagatg gcggaatggg tggaggctct cgggattgtc caggcaggag |
| 92941 | gagtccatgt aggactctgg acctggccaa cctgcaggct gaggaggaga aggagaagaa |
| 93001 | gaagaagaag aagggcactg cggaggagcc agaggaggag gagccagacg agagcatgct |
| 93061 | ggactggtgg tccaagtact ttgcctccat tgacaccatg aaggaggtga gacctcgggt |
| 93121 | agggtgaggg gaggtgaccc cctccacact cagaccctcc tgtccagctt ctttcccacg |
| 93181 | gcccccaaga gctggatccc aactccattc atgtgagttt gggatggttt tgtctcacat |
| 93241 | ccttgaaatc ctagctcaga actcaccttc caagatgatg gtcccatcta ccttccctgc |
| 93301 | ttggttttct tttgtcctca ttgactcatt ccacaaatgc ctcttgggta tatgcgatga |
| 93361 | gccaggctcc atactaggtg ctatgggagc caaaagatgc cacacctgcc ctgatgggcc |
| 93421 | tccggtttac tgggcaaaat ggacatgtaa acagagactt agagaggact atgtgataac |
| 93481 | aaaggcaaat catggcttgg gggaatcagg gaaggcgtct cagaggaggt gacctttgtg |
| 93541 | gaaaaggaga aacatgttct cagaggccat aagggaacaa ctgcttatca gtagtcctta |
| 93601 | cgactcttct tcataatgtt ttattttaca catcttggtc atcggcacgt gaatgattct |
| 93661 | tcccctatta cacatatgtg ggttccatct gtttggccag attgcaagtt ccagaaagcg |
| 93721 | catttctctg ctttcttgtt tgtgtcctct ccacgctgcc tacctagctc agggtgctct |
| 93781 | gactggccac ctgaccagga gggacacatg cctgcagttg tgtttcctca gtggggccag |
| 93841 | agctgatcca gccacatcct ctcccaggcc ctttctgtga cccaagctag gtgtgatatt |
| 93901 | tattagagca cacattaagc tgctgtagca agttagagtg aagtgaacaa gacagcagtt |
| 93961 | tatttctcac ataacatctc agagagtggg ggttaggctg gtatggtggc tcagtggtgt |
| 94021 | caaggactaa gatcctgacc tcttgtttgt cacatctgta aggttggagg tgactcaaca |
| 94081 | tcatgttcta attcaagtgc acaggaaggg ttaaagagga gatgaggcat atgcttttct |
| 94141 | ttttcagggc ccaatccaga agctgcacat atgacttcca ctcacattga tcagaatcta |
| 94201 | gtcccttagc tacacttatc tgcaaaggag gctgggaaat gtaatctttc acaaggtggc |
| 94261 | tgtgtgctca gcaaaacttg taaaaatagg agaacagatc attgaggaca actggtagtc |
| 94321 | tcgtctccca ccatcaagtt ggccaggctg gagctgcaag aggctgcctg ttgtcgtctc |
| 94381 | tactcctgca attttcacag actgccacca ggtggcggtg gcgacttagt tgaaacctgg |
| 94441 | tttctagttt ctagtaagat ttattctcgt ttccagtaag atggggtgtg ctgagtgaaa |
| 94501 | gaaaatatag tacattttct tactgttata cttttgtaca ttaaaacaat ttattaatat |
| 94561 | cactaaaagc aacattaaaa tgcactgtac ttttttaagt agaaaatgta aatatttatt |
| 94621 | gcaaggtgtt atttcttttt tttttaaatt atactttaag ttctagggta catgtgcaca |
| 94681 | atgtgcaggt ttgttacata tgtatgcatg tgccatgttg gtgtgctaca cccattaact |
| 94741 | cgtcatttac agtgggtatt gctcctaatg ctatccctcc cctctccccc agcaccacca |
| 94801 | caggccccgg tgtgtgatgt tcccctccct gtgtccaagt gttctcattg ttcagttccc |
| 94861 | tcccatgagt gagaacatgc agtgtttggt tttctgtcct tgtgacagtt tcttcagaat |
| 94921 | gatggtttct ggcttcatcc atgtccctgc aaaggacatg aactcatcct tttttatggc |
| 94981 | tgcatagtat tccatggtgt atatgtgcca cagatgcata aacccctaag taccactagt |
| 95041 | cacactgtac acattataaa ctttttttaa cccacttaag cagagcacat ggtggacttg |
| 95101 | aaatgcagta gggaagattt tctacttcta gtgcattaag actggtctgg gcccacaatt |
| 95161 | tggggtcaac agactcctcc ccaggaaacc caccttctgt ccttttcatc tcatcttttg |
| 95221 | gcagcaactt cgacaacaag agccctctgg aattgacttg gaggagaagg aggaagtgga |
| 95281 | caataccgag ggtgagccct ggaacctgga actctgcctc ctcctccccg cccctgggtg |
| 95341 | gtcagctctc ctgcccacct catcacctgc ttctcttttc cctcctgcca gccctcccag |
| 95401 | ccttagtagc cagggaagca gcatcagtaa tccatgggca gtactgggca ggctaaggcc |
| 95461 | cttgtccagg gaggctttta ctctgaccag ttcagcaccc acagtgatgg gcagacattt |
| 95521 | agagccacag aacatcagag ctagaaggga cctcagagct cattccatcc agcattttcc |
| 95581 | agactctttt ccatgaagaa aattgatgga tgttctagaa ggaacaaatt agttttggtc |
| 95641 | aaattagttt aggaaaagct gattgaaaca gcttgttttg gtgcaggact tttcataccc |
| 95701 | tttactgtgc aaatgttatt atacatctct aaaaggaaga cacagtataa aatatttccc |
| 95761 | cagcttattg gatcacagta ccctttcttc taaagcatgc cagcagcatc tgcaccaggt |
| 95821 | gctccactga atgaggtttg ggaaatgcta atacagagtg ttattattat ggacaagaag |
| 95881 | agggaagcta gtgaggaaag tgagtgggca aggaactgcg cagcaacagg cctccacccc |
| 95941 | caaacccatg ctccttcctg cccctctctt ccaaagggca tgaagacagt cttcagacac |
| 96001 | cttcccgggc ataaacccac tggggccccc agcacctcca tgaggcgggc atgctaggga |
| 96061 | gtattatctg catttgacag cacatggaca tccgagagtt aagttagtaa agcaaattag |
| 96121 | cgacagagct gggtctcgaa tccaggcctt cctgctctgc tcatttctgc tccacctcct |
| 96181 | tttctgcttc tggccaggcc tgaaggggtc aatgaagggc aaggagaagg caagggctgc |
| 96241 | caaagaggag aagaagaaga aaactcagag ctctggctct ggccaggggt ccgaggcccc |
| 96301 | cgagaagaag aaacccaaga ttgatgagct taaggtgaga gcctaggagc agacccttct |
| 96361 | agggtgtgtg ggggcgggaa gaggggggcg cagtgatcct gcaggtgggg caggacaaaa |
| 96421 | gccagagacc aggcagccgc cccctaacag cctggtctgt ctcaggtata ccccaaagag |
| 96481 | ctggagtccg agtttgataa ctttgaggac tggctgcaca ctttcaactt gcttcggggc |
| 96541 | aagaccgggg atgatgagga tggctccacc gaggaggagc gcattgtggg acgcttcaag |
| 96601 | gtcaggccag gagcacgggc ccagcctgag cacccgccag cccttgcccc ccccacaggt |
| 96661 | tcccccactc acctcccacc actgccccgg ccactgcctc cagaagccct ggactctccc |
| 96721 | atgactcagc agttaaaggg tgaaggccct gccacatcag ggtccagccc tcctggtgct |
| 96781 | gttagctatt ttgaatgcca cccttgccag gcaaacctac accctgaccc acatttgtct |
| 96841 | tgcccagggc tccctctgcg tgtacaaagt gccactccca gaggacgtgt cccgggaagc |
| 96901 | cggctacgac tccacctacg gcatgttcca gggcatcccg agcaatgacc ccatcaatgt |
| 96961 | gctggtccga gtctatgtgg tccgggtgag actcccgtcg ctttccctcc ttgcaagtct |
| 97021 | cctcatcctc ccatctggga gagagcaaag aagctgcata ctggccaggt gcaatggttt |
| 97081 | acgcctgtaa tcccagcact ttgggaggcc aaggcaggag gatcacctga ggtcaggagt |
| 97141 | tcgagaccag cctggccaac atggttaaac cccgtctcta ctaaaaatac aaaaattagc |
| 97201 | caggcacagt gccacgcacc tgtaatccca gctactgagg aggctgaggc aggaaaatcg |
| 97261 | cttgaacctg ggaggtggag cttgcagtga gctgagatca cgtcattgca ctccagcctg |
| 97321 | ggagacagag cgagactctg tctcaaagga aaaaaaaaag aagaagaagc tgcatactga |
| 97381 | cagccccaga ctgggaggca ccagggagat catttggtct agtctttatc ttttctagag |
| 97441 | gaagaagtca acgcccagag aggggtagcc acttgcctag ggtcacacag caagcctcct |
| 97501 | gaggggtcca gtgctcctgc ccctgccctt cactgtcata ggaccctcag gcagcctggc |
| 97561 | aggggcactg aagatgagca gggcatggga gcctgctcag gcagcccttc tccccattcc |
| 97621 | aggccacgga cctgcaccct gctgacatca acggcaaagc tgacccctac atcgccatcc |
| 97681 | ggctaggcaa gactgacatc cgcgacaagg agaactacat ctccaagcag ctcaaccctg |
| 97741 | tctttgggaa gtaaggcttc ttggtgccct cccctgccac acccctactt ctccaaccca |
| 97801 | gaacacctcc caccctagcc aatccttaag cccttctcct aactccaccc caccaggtcc |
| 97861 | tttgacatcg aggcctcctt ccccatggaa tccatgctga cggtggctgt gtatgactgg |
| 97921 | gacctggtgg gcactgatga cctcattggg gaaaccaaga tcgacctgga gaaccgcttc |
| 97981 | tacagcaagc accgcgccac ctgcggcatc gcccagacct actccacgta tgtggggcat |
| 98041 | ggggcagagg gcgggggcag tgtgcctgct ttgcctccac tctaaaatga gattcagatc |
| 98101 | ctggtgattt gagaggttag acattgtggt ttctgtccca ggccaggcct ctgcccctac |
| 98161 | agtccctggg cacagtcttc aggggacagg actgcagatc ccaaaacaag gagagggaca |
| 98221 | gtggattcca ggacagagga ggcaggacga ggagagacaa gtgggctagg gaaggtgggg |
| 98281 | gctagcaaga ggaggacaga aagagtcccc tggggatctg ggccagctct gccacttata |
| 98341 | ggctgtgtga ccctggccaa gtcccttcac ctctctggtt ttcagtttcc tcatgggagt |
| 98401 | ctctgaggtt ccagaggagt gggcacagtg aggggatgca gtggggctag gctggggaca |
| 98461 | ctgatatgtg gtccaaggcc cagggcagga aggttcagcc tccagcagcc ctagtgtctc |
| 98521 | aggtgagcct ggggcccagg gcaggaggaa tcaggctcac ttgctgatga gggcaacagc |
| 98581 | caactttaga gaactggcca cgaccagctg tcatccagga tggcttagtc ccacccagcc |
| 98641 | cctccaagcc tgtgtgtccc ctcccacaga catggctaca atatctggcg ggaccccatg |
| 98701 | aagcccagcc agatcctgac ccgcctctgc aaagacggca aagtggacgg cccccacttt |
| 98761 | gggccccctg ggagagtgaa ggtggccaac cgcgtcttca ctgggccctc tgagattgag |
| 98821 | gacgagaacg gtaatggggc actggatgca gccgcctgcc ccctctccag ggggtttcag |
| 98881 | aggccaggag gggcccacac ttccctgggg aacctcacag cctagcctgg ggtgtcctca |
| 98941 | gcctaaacct ggtcttagtg accctctgct catcacccca gagccccgtt cctgtctgcc |
| 99001 | cagcctctgt gcttccaccc ctcctacagg gcagccccac ctgctttctc tctgtccaaa |
| 99061 | tcccagtgag gtagagaggc tatctgcatc ccattctgca gataaacaaa ccaagatcct |
| 99121 | acttctagtg actcgcccaa ggtcatgtgg ctctttagag gcagagcgag ggcatcggcc |
| 99181 | caggtccatc tggctctaat ccttaaactc tccttacttc tctgtccatg aggcacttcc |
| 99241 | cagagaagca gagaattggg aggtgtctgt gctggtatct gatcactttc aggtgctggg |
| 99301 | acaggaagtg gattctcccc tccctgaccc ttctctcagc ccagcagggc tctccagtca |
| 99361 | acttccccaa gcagaggacc aagggcacag acctctcttg gccctgtggg gcagctgaga |
| 99421 | ccctgggcaa gccacttctc cttctgtgcc tcagtttccc caacagtgaa aaggaggtgg |
| 99481 | ggtagacagg tgatggcata gaggcttctt tgtgctccta agtccctagt cccagcaaag |
| 99541 | gtcttctggt tctgctgagc catgtgtgca gctgagccgc cggcacccac aggtcagagg |
| 99601 | aagcccacag acgagcatgt ggcgctgttg gccctgaggc actgggagga catcccccgc |
| 99661 | gcaggctgcc gcctggtgcc agagcatgtg gagacgaggc cgctgctcaa ccccgacaag |
| 99721 | ccgggcatcg agcaggtgac acttgcatgg ccaagactgg ggttcttggg tattggggac |
| 99781 | cagggatccc caacctgagt cagtaaaggt agaagccagg cctcacgctg tccgctgcat |
| 99841 | tccagaaagc ctggcaggca ccttcccacc aaggcccctc agggtaactt gaatggcaag |
| 99901 | ggtctttgct ttgagtgaga attacatcag ctcaggcaga gacctgggtt gtctccctca |
| 99961 | gaagctgtga ttggctattg gtgacatcat aggaattcct ggcatccaaa gatggaaact |
| 100021 | gatgaactgg gtccttgaca gaacaaaggt taggagggag aggagagctg atcttcctgg |
| 100081 | gcagagggag aaggccctgg gagatctgcc acaaccctct tcctcagggc cgcctggagc |
| 100141 | tgtgggtgga catgttcccc atggacatgc cagcccctgg gacgcctctg gacatctcac |
| 100201 | ctcggaagcc caagaagtga gcggcctggg gcccagcgct actcccttcc ggcccacccc |
| 100261 | acccacttcc caccacggcc caacccttcc tgcggggggc tcaggtccag atggccttgg |
| 100321 | ccagttggca tccctggggc ctgggtgtgg ccactgggcc ttatagggga gaaactgacc |
| 100381 | aaccaatcga tagagtttag ggcctgccct ggagagtccc cacgtgctaa ggcaggacaa |
| 100441 | cgccttccac cctgcatcag tgagcgagac aagccccaca cagcacactc cggtgcattg |
| 100501 | tatctacacg gtctacgcgc ccatacacat gcatctcatg cacacacaca cacacagcat |
| 100561 | agcactccca cggacaccca cacacatgcc cacactgacg cactccttca caacacacag |
| 100621 | aacccctggg agccggccct gcgtcatcct cacatccagg ctagtgtgat gttccgccaa |
| 100681 | gttttctttg gagtgcctgc cagtgatgaa tccaggagtg agtgcctgga agaggcagga |
| 100741 | ggtggtagag tgaaaagaac ccgggtttcc catccataaa gcggcaggtt caagtccagc |
| 100801 | tgtcagacag tgactggatt cagggccagc ttcagggatg gaggaagcca cacagggtcc |
| 100861 | cactcttgtt tccatgctcc tatgttgcca tcttgaaatt ctttatgaca agtgaccctg |
| 100921 | catttccatt tggcgctggc cccagcaaat catgcagcaa attctgccag gaatgtcctg |
| 100981 | gaaacctgag gccatgagtc agctgttcca gggcctgtca ttagggggca cccaactcac |
| 101041 | tgggccactc tgggctcgca gccctgtctg caatcccagg agagccatgc tcagatcaat |
| 101101 | ttttagtgaa tgactcagcc tcactcttaa tgattgaaat gaactacatc attcataatt |
| 101161 | acaattaaac cttgagggga aaatcctctt tggggacagg gacatacagc ttccatgggc |
| 101221 | ttccattgct ggctactggg cttaaatgca gacttctctt ggaactagca tctgagacag |
| 101281 | agggtggctt cctcccctca ccccaccccc accgcccaca gccatgtgcc tggtgggcct |
| 101341 | tctcactcat gcctctgctc ccccaacccc aaaccactcc tccagcccca gcctggccca |
| 101401 | ggccctgtgc tgaccccaag accccagggc ttctctccgc ctgccccatc ccagccctgg |
| 101461 | caccctggca taacccccac ctggggctgc taaccagcct tctctccctg ggcccaggta |
| 101521 | cgagctgcgg gtcatcatct ggaacacaga tgaggtggtc ttggaggacg acgacttctt |
| 101581 | cacaggggag aagtccagtg acatcttcgt gagggggtgg gtgagcagtc cctgcatggg |
| 101641 | agggactgag ctgggtgggg cttgcttccc ggggggcagg agaggaggca gagggaaggt |
| 101701 | ggctgtgggt gggagaggcc ccaccagcct ggctgcgggt ctggagatgt ggcgcccaca |
| 101761 | ggtggctgaa gggccagcag gaggacaagc aggacacaga cgtccactac cactccctca |
| 101821 | ctggcgaggg caacttcaac tggcgctacc tgttcccctt cgactacctg gcggcggagg |
| 101881 | agaagatcgt catctccaag aaggagtcca tgttctcctg ggacgagacc gagtacaaga |
| 101941 | tccccgcgcg gctcaccctg cagatctggg atgcggacca cttctccgct gacgacttcc |
| 102001 | tgggtgcaga gcagggcagg gatgggggtt caggatccag aactgagaag aggcaggggc |
| 102061 | cgggttggcg gggacaggga gagccctgcc ttggggggac aaggggtcat tgcttccagt |
| 102121 | ccatcagagc caggggtgtc cgacggggtt ccgcccccaa ggacccacct aaccgtccct |
| 102181 | aggcagatgt tctcagtgtc ataaccaggc tgacaacaag ctttggacac accaaggagg |
| 102241 | gggcagtcag tgatgatggg ggaggggaga ggctccctaa gggagtcggg aaacctccct |
| 102301 | gggcctcagg ttgcttctct gtaaagcagg gattctccct aaaactctaa tctgcatctg |
| 102361 | caaaagcttg tgggattagc aggactttga cagaatggat gcatggcagg gagtggcagg |
| 102421 | ggagacccca ggagagaccc tctggggtga aagaaggacc tgagtaaagc atgggggctg |
| 102481 | gggctgccta gcaagcccac gaggatgcct gcattgaggc cagttgctgg gagtgaggca |
| 102541 | ggaggatgag atgtgggctc agggaggtct gcactcagca aacaaggggc cgaggagccc |
| 102601 | agacccccac ccccaccccg ccccacccca gccctgttca ctctgagctg acactgaggt |
| 102661 | tgccacaggg gccatcgagc tggacctgaa ccggttcccg cggggcgcaa agacagccaa |
| 102721 | gcagtgcacc atggagatgg ccaccgggga ggtggacgtg cccctcgtgt ccatcttcaa |
| 102781 | gcaaaagcgc gtcaaaggct ggtggcccct cctggcccgc aatgagaacg atgagtttga |
| 102841 | gctcacggtg cgcacccctt cctgctcggg cagtaggcag ggctgggact ggctggggtg |
| 102901 | ccaagggccc agcacgcagc ctggacgctg ggacctgtct gtcctctgca gggcaaggtg |
| 102961 | gaggctgagc tgcatttact gacagcagag gaggcagaga agaacccagt gggcctggcc |
| 103021 | cgcaatgaac ctgaccccct agagaaaccc aagtgagtgc cctgccggcc cagcccctcc |
| 103081 | ccactctggc tcttggaggc gctggaacag gaaggagacg atccccagac accccatctc |
| 103141 | ttcctgggcc acagcctcat tacaggggag cctcctccct gccctctgcc aaccctgaat |
| 103201 | cccacaacgt ccctttgaac cagaggatgg gctaggaata ctggagagca ggaccttccc |
| 103261 | tgggaaaggc cccatctagc ggagagaaac tatgctcttc ccttcctagc ttttgcagaa |
| 103321 | gagccagctc cagtggtggc ctctggggcc atggccagcc ttgatggcca gaggggtgcc |
| 103381 | tagaactgca cacacagccc aggacagctt ccctcttggt cttggcccct cgccacaccc |
| 103441 | aatcccttct cctccctgtc cctcctctgc gctctctacc cttcatactc cagccggccc |
| 103501 | gacacgagct tcatctggtt cctgaaccct ctcaagtcgg ctcgctactt cttgtggcac |
| 103561 | acgtatcgct ggctgctcct caaactgttg ctgctcctgc tgctgctcct cctcctcgcc |
| 103621 | ctgttcctct actctgtgcc tggctacctg gtcaagaaaa tcctcggggc ctgagcccag |
| 103681 | tggcctcctg ggtctgatat ttcttacttc ttcctctgga ccaagggcta ggcaggcgcg |
| 103741 | tgcacacaca cacatataca cgcacacatc cacaaacaca aatatgcata catgtgcaca |
| 103801 | caaacacaca caagcatgta tgcacacatg tgccacgcac aagtgcatac acatgcactc |
| 103861 | aagcacacac aggcacacac ttaagcccac acaaacatgc acagcaactt tcaagctttc |
| 103921 | atgacagaat agactctcag aactagaaat gactatttta cacttttcca cattaagcca |
| 103981 | aagaggtgaa gtagcttgct caaggttacc cagcaaatta gggtagagcc gggactcaag |
| 104041 | ccaaggtttc caaatttcca atcctgtgtt ctttgcacac cacctctgct cccagccatg |
| 104101 | tttctcccag actgtaaccc aatttttttt tttttttttt ttttttggag acagagtctc |
| 104161 | gctctgtcgc ccaggctaga gtgcagtggc acaatcttgg ctcactgcca gctccgcctc |
| 104221 | ccgggttcac gccattctcc tgcctcagcc tcctgagtag ctgggactac aggcacccgc |
| 104281 | caccacgcct ggataatttt ttgtattttt agtagagacg gggtttcact gtgttagcca |
| 104341 | ggatggtctc gatctcctga cctcgtgatc cgcccatctc ggcctcccaa agtgctggga |
| 104401 | ttacaggcgt gagccaccgc gcccagccga ctctaaccca atttcattgg cagaaagaag |
| 104461 | ctgagctatg gacagggaaa agggaagggt gtggccaggg ttagccaaca ggtctggaaa |
| 104521 | tggctctcac ccgtgtgtga cctcccagtc cagggctgtt gttttctgcc cttcaggata |
| 104581 | ataattccat tcaactcaaa catttcatag ccaccatggc ctgaacaccc aactgcaggt |
| 104641 | gctcagagct ccccaggcac tggctcactg tgccattgcg ccaaacccag gaggtaggta |
| 104701 | ctgcttgtaa ccccatttta tacatgaaga aaccgaggtt gatagaggtg cctcacttgc |
| 104761 | ctgtgaaaac cattcaaaac caggtgccct gcacccaagc cccggctctg tgtccccacc |
| 104821 | aatactgagc tcagctctgg ggctgcacgc catgtcctct gggaggcagt agtttgaaaa |
| 104881 | tgcatatgca gtactttcac ttgtatcttg acattttaca aaacctattg tttttataac |
| 104941 | acaaacgaca gatagtgagg ctggccaggt gcttctcggc tgggaggctc tacagaaggt |
| 105001 | ggagtgggtc ttcatcctga gagtgggcag gtcggtgagg gcagcttgca ccccacaggg |
| 105061 | gctgctcaga ccctcagcag ggcccggtgg gaacagccat ttatgtccat cagaaaggaa |
| 105121 | gaggttgtga agcaccagtc ttaccaagga cgtggggcag cacatagaac gagctaaccg |
| 105181 | caggagcttc ctaacgatga gaactgcctg cagggccaag gctgccccat gctgcaagta |
| 105241 | gctcccatct aggccttagg aggagattcg ggccccaaag gtgctgcagg gaggatagag |
| 105301 | ttttgtaagc agcaagtcgg gtacttagaa atgggtttaa accctggctc cagcattcac |
| 105361 | tgcttttggg gctggggcca tggcagggct tttgggggtc cagaaggaca gaggatgggg |
| 105421 | tgcactggga ggcagctcct gcccactggc tgaccggctc ctctcttcca cttcccagcc |
| 105481 | ggcccgacac ggccttcgtc tggttcctca accctctcaa gtccatcaag tacctcatct |
| 105541 | gcacccggta caagtggctc atcatcaaga tcgtgctggc gctgttgggg ctgctcatgt |
| 105601 | tggggctctt cctctacagc ctccctggct acatggtcaa aaagctcctt ggggcatgaa |
| 105661 | ggccgccagc tcccgccagc cgctccccag ccctgccgca tttcctttca gtggcttgga |
| 105721 | ctctttccca tctcccctgg ggagcctgag gagcccagcg tccactcttc atgccttggg |
| 105781 | ccgagcctgc ctcctgcttg cgggggccgc ctgtcctcac tgccccaggc tgcggcttgc |
| 105841 | ccagtcccgc ccctctgacc cctgcctgtg ggctggggag ccttggatgg ggtggggacc |
| 105901 | tggaatgggt ctctcttgcc ccacctggct gaggcgccac ccttcttcag gcccaggctc |
| 105961 | cagaggaaga ctcctgaaac cctccccagg tcttccaagt acaggattga agctttagtg |
| 106021 | aaattaacca aggaccatgg gtcagtgccc agggctttaa aaagaatgaa cgagcaaaag |
| 106081 | gtatccccgc cgtgacccct gcagatagca ccggtctttg atccgcagca ggggccagac |
| 106141 | cctgcccaca agtcccagcg cggctgcttc tgccactgct gggctccact tggctcctct |
| 106201 | cacttcccag ggggtcgcct gtcctgcctg tgggtttcca tggcttccca gagctccctc |
| 106261 | tgccccagcc agcgcctcca ggcccagctg aggagctgtg agaagcagca gaggggactc |
| 106321 | cccatcccgg gcacaccctg tcctcccacc cctgccccct tgcccttcca gccctttcag |
| 106381 | ctgcagctgg gagctggccc gtcaagtgct gcccctgcct gtgtctgggt ttctgttggc |
| 106441 | tgtttttctt ttcttgagtg gtgatttttc tctaaataaa agaagtcaag cactgagctg |
| 106501 | acaggtctgc actggtcaag ctgagaggag cagccgcagg tgcggggcag aggcagcagt |
| 106561 | tctctaggct ggcagattca gtaccagcag ggctcccatg gcctttcccc ttcaagtggc |
| 106621 | ctctggtggc ccagggttca aggcaggcct caggcacagg gctgcccacc caggctgccc |
| 106681 | acaggaggag tcttacacca tagcccaaag ggcacaccag cctgggccca ggggccccca |
| 106741 | aagcaccgaa agaatccttc aggtctgggg ccagtctgaa ggcttaaggc cccacaaggg |
| 106801 | cccaaaagct ctagcctcca cccacagaaa ccagttcccc tgaaagcaag aaaggagccc |
| 106861 | cacgctactg gcctcggcct ctgactcaca ccactatctg agcctggggc aaagtcccct |
| 106921 | catgacacag aggtaggggc aagggcctgg tcttgctaga cctgacctct ttaaagagat |
| 106981 | tcagccacaa ctttaaaagg ctttaatcat aatgcaggac acaggtaact cctctggctc |
| 107041 | cttatttcga caggctataa agacagccct ggaaaatcca gagagcaggc ccaggtgcgg |
| 107101 | cccagtgggt gatatgagct ggctccggca cagacaccag catcaggcca gccctaacac |
| 107161 | atcagccttt ggcggtccag ctcattttgt gggtacccgc aacacctgag tgggaggaac |
| 107221 | ttgcagttca gagttgatct ggaaaagaaa gggtgaaagt cgttacattt ttcccagggc |
| 107281 | cctccttctt tgccaccctt gcgagccacg ctggctctcc catgggcctc caaagccctg |
| 107341 | cacgcatctc ctctccaaag ctgagagatg ctgaacaccc acagactgcc tccctgcagg |
| 107401 | agacacgcct gagccttctt gctcagcttc tgctccagct gtgacctctg gtagcgttct |
| 107461 | tcatctcccg cccgtccatt ctctgcagtc ttcccctctc ctctctccac gcacccatgc |
| 107521 | catccccaat ctagcctgcc ccacctcttc ctcccccagc ctcttgtata cagtagcatc |
| 107581 | tgtggaagcg ggaaccatac cgtccagaat gtctgggtcc tgtcacaggc caagggtcat |
| 107641 | tatgaggcca aggttcacta caacatccca ctttcagctg tggcctggga cctgggactg |
| 107701 | tgctggggcc acgcagcccc tgtggctgga cttggagctg gcctcagtca cctcacacgt |
| 107761 | gtgcaactaa cagctgcgtc acgaccttcc tggcctacag ccccagctgc aggtgcccac |
| 107821 | acccgcagag caggaaatgt cagcaatgga ctttttcctt cctcctcttg ccaggccatg |
| 107881 | gggaagttgt ccacaactat tgcagggttg ggcacagatg ggggccaggg aatatctgcc |
| 107941 | ccagcagagc ctccttcccc atgcgaggat gaccctccag tcatatccca ggcagggaac |
| 108001 | cggggctggt gggtggaaag agcccaggct ctggacgagt tgcaaactct gcaagttgtg |
| 108061 | attgtccttt gtgtgaaatg aggagcattg tttccgccct gtccacctca cacagcgggt |
| 108121 | gtgaggatgg aagaagagag cacagctgga ggctctgtag ctggggagct gctcctggct |
| 108181 | gtggcactgc ttcacacaga gcagtcccag ctggccactg gcaccagaaa ccacagtatc |
| 108241 | aaagagacac tgtcacacct gagctgtcct ggaggagact tcaggcaaga tctactcctt |
| 108301 | tggccagtag ttgtctcaag gtgccactct tgatttttta catcatatca caaagctccc |
| 108361 | ttgcctctga cggggagggg actcctcgtt ccctaatccc agctccagtg cccctgcccc |
| 108421 | acccgtgtct cccgctgccc ctccttggaa ggccccgccg cccaccttgg agttcagata |
| 108481 | ctgctgcagt agcgcc |

### Norrin Cystine Knot Growth Factor (NDP)

The NDP gene encodes “norrin cystine knot growth factor” (NDP). The human ND gene is located on chromosome Xp11.3. It contains 3 exons, encompassing kilobases (kb) (NCBI Accession No. NG_009832.1). NDP encodes secreted norrin protein that plays a role in retina vascularization of Wnt signaling pathway through (FZDA) and (LRP5) coreceptor. Mutations in NDP have been associated with Norrie Disease Pseudoglioma, an X-linked recessive syndromic hearing loss that is characterized by early childhood retinopathy. About one third of individuals with Norrie disease develop progressive hearing loss, and more than half experience developmental delays in motor skills.

Various mutations in the NDP gene have been associated with hearing loss (e.g., Norrie Disease Pseudoglioma). For example, the p.His4ArgfsX21, p.Asp23GlufsX9, p.Arg38Cys, p.Ile48ValfsX55, p.His50Asp, p.Ser57*, p.Cys93Arg, p.Lys104Gln, p.Gly113Asp, p.Arg121G1n, and p.Cys126Arg mutation have each been associated with Norrie disease pseudoglioma. See, e.g., Musada et al., Mol. Vis. 22:491-502, 2016; Chamney et al., Eye (Lond) 25(12):1658, 2011; Liu et al., J Chin. Med. Assoc. 79(11):633-638, 2016; and Parzefall et al., Audiol. Neurootol. 19(3):203-209, 2014.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype NDP protein is or includes the sequence of SEQ ID NO: 20, SEQ ID NO: 21, SEQ ID NO: 25, SEQ ID NO: 26, or SEQ ID NO: 27. Non-limiting examples of nucleic acid encoding a wildtype NDP protein are or include SEQ ID NO: 21, SEQ ID NO: 22 or SEQ ID NO: 24. As can be appreciated in the art, at least some or all of the codons in SEQ ID NO: 21, SEQ ID NO: 22 or SEQ ID NO: 24 can be codon-optimized to allow for optimal expression in a non-human mammal.

Human Mature NDP Protein (NCBI Accession No. NP_000257.1)
(SEQ ID NO: 20)
MKTDSSFIMDSDPRRCMRHHYVDSISHPLYKCSSKMVLLARCEGHCSQASRSEPLVSFSTVLKQPFRSSCH
CCRPQTSKLKALRLRCSGGMRLTATYRYILSCHCEECNS
Human Mature NDP cDNA
(SEQ ID NO: 21)
atgaaaacggacagctcattcataatggactcggaccctcgacgctgcatgaggcaccactatgtggattc
tatcagtcacccattgtacaagtgtagctcaaagatggtgctcctggccaggtgcgaggggcactgcagcc
aggcgtcacgctccgagcctttggtgtcgttcagcactgtcctcaagcaacccttccgttcctcctgtcac
tgctgccggccccagacttccaagctgaaggcactgcggctgcgatgctcagggggcatgcgactcactgc
cacctaccggtacatcctctcctgtcactgcgaggaatgcaattcc

A non-limiting example of a human wildtype NDP genomic DNA sequence is SEQ ID NO: 22. The exons in SEQ ID NO: 22 are: nucleotide positions 1-87 (exon 1); nucleotide positions 88-468 (exon 2) and nucleotide positions 469-1719 (exon 3).

| Human NDP Gene Sequence (NCBI Reference Sequence: NG_009832.1) | |
|---|---|
| (SEQ ID NO: 22) | |
| 1 | aagatgctcc gtggaaggga gccgagcggt gggcagaggc tgagtccccg ataacgagcg |
| 61 | cctcacattt ccgtggcatt cccatttgct agtgcgctgc tgcggccgca cgcctgattg |
| 121 | atatatgact gcaatggcac ttttccattt gacattctct ctctctctct ccctctctct |
| 181 | ctctccctct ctctctccct ctctctctct ccctgtgtcg cttaaacaac agtcctaact |
| 241 | tttgtgtgtt gcaaatataa aaggcaagcc atgtgacaga gggacagaag aacaaaagca |
| 301 | tttggaagta acaggacctc tttctagctc tcagaaaagt ctgagaagaa aggagccctg |
| 361 | cgttccccta aggtaagcag gaaaacaagc gctgccggct tgcctaggaa atcgcttctt |
| 421 | tccaaaccga gcatttcatc ctcctctcag acaaagagac aacctttaaa taccacttaa |
| 481 | atttgcatta ctttaatttg cattaatctg tagcatgcta gcattgtgtt tcttaaatga |
| 541 | acgggtcttt gaagtctgtg acagagcagg gagcctccta gaaaagcttg aaaatatctt |
| 601 | ttggtttgac ttaaagagat ttttcttttc aaactctctg cgtagttaga tgtaactttg |
| 661 | tgatggttct tatagttgac cccgtgaaag ggggctgcga agcagcaact tgccaacatg |
| 721 | gattcatgga gcccagtatc ttccccagca ccctcatccc acttacaatg ggggcactat |
| 781 | taacgcaggc aagactgcga gtttaaaagc gcactgttta cttttattac tacttatgta |
| 841 | tgctgctgat ctcctgccag ggctctgccg gagtgctcag aatgacttca aacaataggc |
| 901 | aacatttatt tctagctttc tgtgtcacgc tttgcaggaa tcctgctccc ttgcactctt |
| 961 | ctttcagcgt gcaggtcttc gcttccatag atgcattggg gagcaccggg aggagatgag |
| 1021 | gatggattta ctgtatgatt ttgattgggc aaaaagtccg aggaaatgca tgctgtttgc |
| 1081 | cttggactta ttgcaggggg ttatttttag tcatcagctc gtccctgcag catgatgttc |
| 1141 | aaggagtgac cgggaaagga tgctcccacc agcacagaga ccaggacctg taggaatgac |
| 1201 | tcctacctgc gggatattcc gaggtttctt tcaaactttg gaatacaagt tctctcttta |
| 1261 | aatgaacaat tgagctgtag gaggcctggg cataactctt tcttggggga cgggaagttt |
| 1321 | gattgtgtac agaatgttaa tgactcagaa cgcccagtaa ctgaggctca ttgtaataga |
| 1381 | caaacccaga taagcctcca tgagaggcag actcccatct ttactgagat tcaggtgcgg |
| 1441 | gaaaggaagc tatttggcaa gatgggagtc ccttgtacag ttctttgtaa ctcattaata |
| 1501 | acagatatgg ctgctgtgaa acatgtctct tttcatttct aatattagtg tattttaatt |
| 1561 | acaaggagct ggcattggca aattacccca acacataaga ttaaatacca ggtgcatttg |
| 1621 | aaaacagaga gatgactgaa atggggagtt gctgtgggtg tcaaccaaat acagcgggga |
| 1681 | cggcacaatg cttctcaact gagcgtttaa accagatgca aatatctgct cattttcttc |
| 1741 | tcatcagtct tactaattca aggaaacttt atggtcctgt ttgccagttt tcattgcaaa |
| 1801 | ttggtacaca gtttatgcaa aacttttctg attttctctg ataaccagcc caataagcat |
| 1861 | cgctgaatga aaatctgata tttgaatagg ttaatttaca actttggggt attcattagt |
| 1921 | caagaaagcc ataaaagaca ctatggattc cattgctgta aaattttatt gtaatttttt |
| 1981 | tttaaaaaaa ctgcggttag caaagcattt agctgaatca aaacaatttt cttagactat |
| 2041 | tattcagatc attacatgag agtatttgaa cacaaaatgc aatgttttgc atcatttatt |
| 2101 | ttaaaataca tttggttcag gtaacgggag aactggctga ggccttctta cgactgtgta |
| 2161 | ccaagcagct gacacccaca gatggaaatg tgcctgtgga gttttcaatc ttaattcgag |
| 2221 | ttccaatgac actgtcaagg gaaaaataaa tcatttttca atacatttaa caatttccaa |
| 2281 | ctcatttatt ttcctgggcc cttgaaaaat gtggtgagtt tcttggaatt atagccacca |
| 2341 | atggtggcca gcaatcttta ttttaagaac tcacaattca agtactatta ttctctggtt |
| 2401 | aattatttat ttcatcaatt tttaaataaa tatttattaa gcattttcta gatgcccagt |
| 2461 | gctatgctag tcccgatgga tgcaaaggtg aatgagacat tgtcccatac tctaattctc |
| 2521 | agtccatatg ccatctctag ttagattttg gaactgaaat gggcattgcc aatgggtaaa |
| 2581 | gtgggcaata actttcctgt tataaagaat tatttaaaac agctactggt ttctagattt |
| 2641 | acttgaaatg attctcactc atcagtgggt tttctttcca tttatgatta agggattccc |
| 2701 | ctttggcctt tttcaccaga tcctgggtac acgtcatttg cacagctaac acggctagat |
| 2761 | ctttccagcc tgggtagaag aatcccggaa aaagacagta gtttatcctt tctctgctct |
| 2821 | cctgagcacc ctgtggaaag gaccatatgc tgtctctatt ggggatgggt gtgagtcgcc |
| 2881 | tactcagtag ctaacatcag ggccacttga tgaatcatcg aattagaaac atgcatatgg |
| 2941 | aggaagatgc agtcactatc cctcaccact tacttgtccc acccccagta tcaaaacaaa |
| 3001 | tgacctctgg gcctggccta aattcaagag gcccctggga agaagctgtt ggaaactctt |
| 3061 | ccccaaaccc aggtctccat catagccaca cagtgttgct ttcaaataac ccagtatggt |
| 3121 | gttttatcac gtgtttaggt tactggggga aaaaaaaaac cctgaaaaca gcatcaccag |
| 3181 | ggtccttgac ttggtcctta tcagcagata atcccccgtg gttggattaa aatgcctctg |
| 3241 | actccctggg gtgcctgact caccctagcc agccctcagc ttcttggcca gcggccttcg |
| 3301 | agcccatcct cacctcttcc catgacggcc agtgactgct tctttgccca ggccctctgc |
| 3361 | actaggccag cagccagtgg gagcagaggg gatgggaagg caggtggaca gactgtgaag |
| 3421 | tgctaaggca gagcggggaa gcagggctgt gagacagggt gggtgagctc cagggcctgg |
| 3481 | gttgctgtgg cctgccctgg ctgtgatcag ctgtcatttc ctctttgtgt atgacactgt |
| 3541 | ggaacatatt aaaatttctc tgggatggta tgtttggaat tcctctgatg ccccacctca |
| 3601 | ccaacttacc cggacaataa aagcaaggtt gtaagccttt gaacagctgg ctggctatat |
| 3661 | atttcccttc tttctttcgc ataatctaat tcagaaaaga atccagctag actggcagtg |
| 3721 | ccatgtctgg gtacttgttt cctgcaagtc cttcctcctc cacctcctcc cgccagtcct |
| 3781 | ggaatcttcc tgggtggcat gtggcttttc ttttcttttg ctcaccactg ctcccagccc |
| 3841 | gccccctttt cccttcgcct gagctccttc ctttgtcagc tggctctttg ctctgcctgt |
| 3901 | tttccagaca gcttgggtcc tgttccatcc ggggagaaca gcccatggga tctccccatc |
| 3961 | tccatagtat tcccaggatg tcattcttgg caccagctct gccgggctga taggactggg |
| 4021 | gggctttttg tagtcccgag cctgaatcca ctcacagaga ggagcagcca catggcttag |
| 4081 | ctgccctttg ggctgccgag agcggggctg gagtgaccgg gttggcagga acgagcatac |
| 4141 | catcacagtg acccgaacat ctccggcaaa ggccttgggg tgggagcgct atgggaatgc |
| 4201 | ctggatgcac ttcggcctac attaagagaa atgtggttat tctctgtttc gggggagaga |
| 4261 | caagcggctt gaaacaaagc cctctggatc cttggcccct tccctgcccc ctttcccggt |
| 4321 | ctgtccttcc cacatccctc cctccccaaa cagaaagaaa gagagacagg acttctaatt |
| 4381 | tggccagcac agacccgaat gtctttatga aacccattta gtgaaagaaa gaggggagaa |
| 4441 | atatattctg ggttgatccg aactaaaaaa tgcatggggc tttggagatg gggcgggatt |
| 4501 | ttttctttct ttctttctgt ctcttaagag cagaacaaca gtttcgttga tgagttgcaa |
| 4561 | atggaaggaa gtggacctct caattagaga ttcccattta agagcctcaa tgtaaacctc |
| 4621 | cctgtgacat ccttaaaaga tgctgctaat gtccctggga ccaaaaggta ttaaacagca |
| 4681 | agccaccagg cggcttccta ttccttttct ttttctgggg gctttggtcg tctttttcaa |
| 4741 | attagctaaa attcattaaa ggacccaaaa ggaaaaattt aaagcagctg aattaaaatg |
| 4801 | agtctttccc tgttagcctg acaagaagaa aaaaagaaag gaagaaaggg aaagaaagag |
| 4861 | aagaaagaac caattcccct ccactaccac ttcttgggtt ttaatatgaa ttattccata |
| 4921 | caagtcccta ggctagagga tctatttaaa gacgcctggc agcatgaagg gagagggtaa |
| 4981 | aaatacaacc actcttttgt ccatcaaagg ttttctttgg gaggacatta agagcattcc |
| 5041 | tgttttcttg tctgcccctg ccagtggcaa tgttccagct ggtagtatcc actcctcgcc |
| 5101 | catcaggtca gcaatcatga acttctgtgt ttaatttcag gagaaaacaa agggtgggaa |
| 5161 | atccttccga aacccctatg tgtgtgcagc agggcagggg aggctggaac tggaatcagt |
| 5221 | actctcttgg ctgtgcaggt tgcatgctgc aaattcacac acccacacag ttgagttgtt |
| 5281 | gaggaagatt gtgccgatgg cattatttct gatgccagct tcccttgcat ctgcaaagct |
| 5341 | tttttcttgt gtctctggca gtcatttgct tgtcaccatt catgtgcact gtccccttca |
| 5401 | tcagaggttc tcagagcctg atggtcaagg atatgtcact agagtcttac ccccagaccc |
| 5461 | attttcaaca gtaagaacgt gaaaccctgg atggggtttg caatctgaag caactacttt |
| 5521 | cttgttagga agagtgctga ctcagaaaat tcaaagaatg ggtaacttcc gttggcgaca |
| 5581 | tatccccagt gctcataatc aggaattcat ccagttgggg tccatttcac cctcatggca |
| 5641 | aaatctggac ataaacattg actagtagct ttaaactggg tataagattt gcaacagata |
| 5701 | acatttttgt cttttctttt ttcttttttt tttccacaaa agctaagaat ttttcagaca |
| 5761 | atttgctctc ttccaaggcc tggatcaatc ccaacttttt cttttaagcc atgatccaac |
| 5821 | caattgcagt atcctttaaa aatgaaaggg caacaggcta agacattcaa gaaagttgat |
| 5881 | aagcbgtcag aaaatgctca agggtggcct ttctacagtg ccacaaacca acaacagccc |
| 5941 | taaacgtgga gtcctcttcc atcctttcat cagtagggaa taaaaatagg gagagagcag |
| 6001 | gtcaggggag aagtaacaaa agagaagtaa gaaggattat ttttgatgtt caaaatctgc |
| 6061 | tgctgccctt tcaatatttt caaatatcac aatttcagaa agggaaggat ccctttattt |
| 6121 | cacccactct ccctcccagg aaggtgtttg aagttgctgt ctttacctga aaaggaggtc |
| 6181 | aactcctcaa gtgaaaaaac atcccctatc ttcaactcga agtaaaatgg aggtacacac |
| 6241 | atttgtacct aattatgtgg gtacaaatat aatttgtatc cacaaactat aaaggatgta |
| 6301 | aagaatcaaa gaaatccaat ccttaatcaa agtccaattt ttctttaaaa cttgagagga |
| 6361 | tattttaggg ctcattgcat tgctgtgatt tcactgcccc ccaccaaatt atgaaaaatt |
| 6421 | actccttttc agaatttaaa tttttctttt gagatgtgga atccaaaggc agcagaatag |
| 6481 | tattgttaca gggaagctgg cagtcaaaag acatagacat agctgcagca tgcatggttt |
| 6541 | aagcaaatca agacaagcag aaaaccacca ccccaacaag ttcagggtta tcagcaatat |
| 6601 | agggacacgt atttttatgt gatttttatg agaaccttgc ccctttgaca tatgtctgag |
| 6661 | tctaggagca ttccggattg caacgggatt gaattcagta ttcaaatccc accattgggc |
| 6721 | agaaaaggca aattgaatgg ggaatggaaa gactgagttg ctcctattgt ttatgcccca |
| 6781 | ttcctaacta agggctctac agctgttagg tgagatcact acccaccatt ccaaagtgtt |
| 6841 | gttgcjggtg aggaggttca aacgcagctt tggaaacact ttgctgcact ctttgaagaa |
| 6901 | aggccaacat gctcatttgg ctctagggta aaagagagcc tgtaccctga gctaacattg |
| 6961 | gtcacctctc ctcctgaggc cataccacag ccagcagtac agcagcagaa caagtgttta |
| 7021 | gttgtttcag cacaggtatc cccagaacac ctggcacctt ttcttttcca cagggagaat |
| 7081 | gtacattcat gcattttggc aagactgagt cctctgagtt gggtgtctcg gaactctcct |
| 7141 | attttccttc taaagggtct tatgtaaggt gagcagtaga gbitgtctca ttcctcataa |
| 7201 | aaaatcccct tcctgggagg gagtgggtga aatgaaggga tccttccctg actgaaattg |
| 7261 | tgctatttga aaaaattgaa agggcagtgg cagattttga acttcaaaat gaatccttcc |
| 7321 | tcctcctcct cctcttttgt tacttctccc atgacctgct ctctccctac tttaattccc |
| 7381 | cattgaagaa aggatagaag aggactctgt gtttagggct actgttgatt tgtggcacta |
| 7441 | tagaaaggcc acccctaagc attttctgac agctcatccc catctcagtc acagctttta |
| 7501 | tttgggtagc tggatttaca gaaataatca aatccctaga ggaaaagact tcaacagaaa |
| 7561 | agtacctcct gacacttggg taacctgact tggtcatttc caatttatgc aagatttgta |
| 7621 | ttctaaaagt caattagtta tgtcaactgt taaaaactca gaatgagtct ttccattctt |
| 7681 | cagaatgact cttttctgat tatctgtggg ccatagaagc ccacagatac tcagcacagg |
| 7741 | atccaagtgc cataccaagt ggtcaactct tttagacgta agtctcagcc cagggcttct |
| 7801 | ggaatgtacc tcccctagca gagaagctct gtagtagata ctttaccaag cacagggcct |
| 7861 | cgctcttgac cccaagacta ctgattctac ttgcagagaa gacaactttt ctaatcaatc |
| 7921 | tttctcttct atcctgaacc agagtatcag agattgtccc attagggcac gaggatctgt |
| 7981 | ggctcaagct cagttaagaa ataagcgtct ttcaaaagaa aggtgcaaga gggtggaact |
| 8041 | cagcctatat tgtcattatc aaatgcaaga agtctaatcc tagcatgatt tattaatatt |
| 8101 | agcctttctt ctctccccgt ttatgctttg gtgggtactg gacagaaacc ccacaaattt |
| 8161 | taagacagtt ttaagagaaa tagtaactgg ttaaatatcc tcactacttc aaattattat |
| 8221 | tattattatt gatattactg ttaacttttg tttttttgag acagggtctc actctgatgc |
| 8281 | ccaggctgga gtgcagtgat gcgatcacag cccactgtag gctcaaccac ccatgctcag |
| 8341 | gtgatcctcc cacctcaacc tcccaagtag ctgggatgac aagcgtgtac caccatgcct |
| 8401 | ggctagtatt tttttattat ttttgtagag atgaagtctc cctgbittac ccaggatgat |
| 8461 | cttgaactcc tgggcttaag tgatcctccc aacactgcct tctaaagtgc tgggtttaca |
| 8521 | ggcatgagcc gctgcacttg gcccacttcc tatgattaac aggatattat taatttagaa |
| 8581 | actatttagt acatggcaag cactcagtca aatggttatt aacggcbgtt tttatttcag |
| 8641 | gccaccaata tagcttcatc aaggggcaaa tacttatagc ttgggcttca gtgttggatc |
| 8701 | agacaccacc ttagaagttt tttgcaatca ttaactcctt aagtaagaag ggaactcttt |
| 8761 | ttgtaggatt gctttgggga atatgtagcc ccaaggcagg ggctgtactc tgagaagctt |
| 8821 | gggtagagtt ggagactgtt agaaatggat agacacataa aagcatttag accaatctgt |
| 8881 | tcattttgca gatgaggaaa ttgagtccta gaaaaggcaa gtgacttgtt caagatcaca |
| 8941 | cagccaatga tgggagggta ggactcaggg cttgaggatc agtttcagaa tgcagtgaaa |
| 9001 | ttgttatccc atatttcttt caaatcagta tagggacacg gaacactaac aacataggcg |
| 9061 | accaatgggt ctagggagga aaagatgaaa gctgtaggta agtgggagtt tggataacaa |
| 9121 | tgagatgttt ttctggagtt cctgacaatg cctgaaaaaa aaagtcagga ttttaagctt |
| 9181 | cagtcaggct caaggtcgag tcctcccact ccagtccctc ctcagacagt aaaacttttc |
| 9241 | aagccaagga catacatata ggtgggggca tttgacagtt ggctcatctc cccaccctcc |
| 9301 | aaattattta tatttctagt ccttcagttt agcactccta tttaccccag gattgtaatt |
| 9361 | cattgcctct ttccctggcc atattcctcc atcctttttg tttctcccat ctcttttctt |
| 9421 | cctccatcac ccccactgct gagcttggct acacttggct agtagtgcag gttgggagaa |
| 9481 | cacagagtcc tatcacagac tggaggacta ttatttaatc acaagaatgt tgtctttttt |
| 9541 | ctcctaaggt agcactctgg ggacggtcat tgctgtcttt attccctcac ccccccacca |
| 9601 | cccctgcact ggattagaca catgaaatgt gcctatctct gagatgaaag gagcaagaac |
| 9661 | tgcttcctcc ccactgacct tttgcctgcc catccttgga gatacctgga gttggcaggc |
| 9721 | tggaggccta gcagcatttg cagtcagtgc cgctgcctct gggtcagccc aggcttggct |
| 9781 | ccttggctgc tactgatgtg ctgccagcta ggaatattaa aaaccattca ttgaacgcac |
| 9841 | aggatgcttt ataaaatcca acatgcbgtt tacagtttat aaattaactg ccttgggaag |
| 9901 | aggcatttga cttattctcc aaggcatcta gttgactaga tccaattgga atcctttgag |
| 9961 | ttcctgggaa ggggctactt aacacttcac actttctaaa ttagacctag agtttcttgc |
| 10021 | ttttctgatc ctagtactgt atgaagtttt atctagggtt atctagcaag ctttcttcca |
| 10081 | cttataaatc tttgatgttt ttgcaatttc tttgatagag gctttatgtt ctactatctt |
| 10141 | cactttattt atgcttcata ccataatttt ttaaaaaatt acaataaata tcaagtaatt |
| 10201 | ctggctgata tactgactcc attccaaggt cattatattt tatttgctta acttggtctt |
| 10261 | cttagaaatg atgcaatgaa tactggatac aaattagatg ttcagagagt ttatcaatgt |
| 10321 | gtctaaaaac catccaggaa aacaacaggc caagactctg cctgcagagt cagtgagcta |
| 10381 | gaagagggat cgaggatagg aactatattt ttcccctgac cctatctctt atctgctaga |
| 10441 | gcaaaaggag acacacagta ggtgctcaat aaatacatgt taaaaaatat ttgttgaaag |
| 10501 | aagccacaga agggaatatc agtatacatt agtattgaaa acagcctagg gggaaatatg |
| 10561 | gatactgaag caccaccctc ctgtgcctgg caagcaaggc aagaataagg ccattcttgg |
| 10621 | cacataccac ttagcctgtt gctgagataa cttgcgtgaa accagcactt taccctctgc |
| 10681 | cagccagaag gtcagcccaa gagtgagctg atctgtgggc acaacaccat ctctcaccca |
| 10741 | ggaggcbjgg cccgctgtaa gctctgctat attatttccc tgaggatgac aggtggggaa |
| 10801 | atctgggcac cagttgttct tttccactgg tatcatttgg aaggtagctc caaagtcggg |
| 10861 | cacgccagcc tacgtgcctc tcctgccggc agaggctcca cattggagct attagaatgt |
| 10921 | gggctctgta cgtcjgccaa gtccatgtct cagagactgt atccagagca tagcgtggcc |
| 10981 | acagggctac tgagttcagc aagagagtca ttctcttgtt caatccatta ttgcctgggc |
| 11041 | caaggttcta agtcttcgta ggcagcattt gattttatga cacgtcttaa aaaattaagg |
| 11101 | caaactggaa ttaatttagt tttgagaaaa caacagaagc tgctacaagg cagagataat |
| 11161 | tctaggctgc cagaaagtgg tgatctcagt ggtgtacatt ttcttaattc ctgattttcc |
| 11221 | tctttgtgag gtcttcctcc cactcccata ctttgcacat tctcacctat gcacaccctg |
| 11281 | agcaacacaa tgttgacaac ttgacttagc attgctcttt tcttaagctt tgttttcatt |
| 11341 | tcttcttcaa tacaacagtg ttggttcatt caatttgaac actcaaggca aaaaaaaaaa |
| 11401 | aaaaaatccc aggttatgtt ttcagaatct tacctttttg tttttgtttt gagggttctg |
| 11461 | tgagttttgt ggcatattta cattgttgct tacattctta tttcagaatt taagataaaa |
| 11521 | actgggcaac aactctcacg ttttctgagt gtgttgaaat gaaaaatctg tttcttagaa |
| 11581 | ttaatggtaa agcaacattt cctgtcaatc tttcattctt taatattact tctgggttca |
| 11641 | gctcaccatc tctaaagaga aagccagatg atcaagttac attgtatgtg acattaacta |
| 11701 | tctctttatc tgtgtaaatg tcataatatc tgcttgcaaa gtgttttatt catttttagg |
| 11761 | aaaaaatttt aaactgtgct tgcaattcag gatggtgcta ctaggtggca gtaatgtaca |
| 11821 | agaactatgt ggtcgccctg gaagagtatt tgactaagag actagcatgc cttcccgtaa |
| 11881 | ttataagaaa taaaaatata atatttttaa aatactcaaa ctggcacaaa cagtaaacat |
| 11941 | ccattaactt agttaccata tttaagctga gcttttaaaa gagaaagcct tattttttaa |
| 12001 | tgaaatttta aataaagtat acattcacaa gctcaaaaat ctaaaaatac gaaaagatat |
| 12061 | atagtgaaaa gtctttctct catgcctgtc cctcaactac cacaggtaac taattttgtt |
| 12121 | agtttcttgg gtatcctttc agagctttgc tatacaaata tgtgttttta tctctctcca |
| 12181 | ttttaactca aatcaataca tagagtttct gcatccttcc atatatagtt ggttagtata |
| 12241 | ttgttgtgtg aacattttag aatataatta accagtcccc tattaatgga tatttcatgc |
| 12301 | tgtttccaat tttatgctat ttcagccatg tgctcatgtg atttcacatg taggataatt |
| 12361 | cacagaagtg gaattgctgg gtctaaatta tatacatttg catttttaaa aggtattgcc |
| 12421 | agattggcct ctacagagtt tggaccaatt tatactgaac tttctccaaa atggacttgt |
| 12481 | tttcccacag ccttgccaat agggtgcatg atcagacttc ggaatctttg taaatctgat |
| 12541 | tggttgaaca atgtatttca aagtacttta attttcattc tcttaaatga gacttttatt |
| 12601 | ctcttaaatt ctcttctatc tttaagggcc ctttatattt cctttttctg tggagaaagt |
| 12661 | ggaaatgcca ataaatacaa cgatgcatac acatgtatag ggttttgtta ttgatgtagc |
| 12721 | ttgtttttgc catcatgctg tacatgatgt tctttaattt gctttgctca tttaaccata |
| 12781 | cctcctggcc atggctctga agagaagttg ctggcagtgc agaagaaggg ccaggactgg |
| 12841 | aatgtctgct tctctcttca tccttttgcc attgactcct gggggaaata tctgtagacc |
| 12901 | gaagcagtga aatctaatca attctctttc tgacactaat taaggcttca aagagacaga |
| 12961 | taaccacttg ggataattta gtcacagtga aacttgggaa atggtcagta atatttcatc |
| 13021 | ctctgaaagt ggaccccaag aaagttctgg ctctttgagt tgttgtcttg gtaattttca |
| 13081 | gggtaacgtg tataggaaaa cccagattat gcagatgaaa agaaaacaaa catgctcttc |
| 13141 | tgcatccttt gcagatttga aactttgggc acaggtttga tttatttcaa tgagatgttg |
| 13201 | ctattagcat gaacataaag tggtgcaaag ttgtgctgga atatggtctc gtcaactttg |
| 13261 | ggctcactgt ctgcatatgg agatgatagt aactgaatcc atagttatca agtggtggca |
| 13321 | ttttagaaca caaagggaca gcagatgcca tctctgtcaa ctccttccct ctacagatga |
| 13381 | ggaaacttgg gctagaggag gttgaatgac ttgtgtaagg cttccccagc taattaatgg |
| 13441 | cagtgccagg actcaagtcc aggtgggttc cctcattact ggtccttgcc cttcctgcaa |
| 13501 | tttataggac ttccctgagg cacatgtgct gaggctttgc tattgccagg gattctgtgc |
| 13561 | tctgaaacag ccctatctca cacacccctt tccacttagg atcccctacc tcacacttcc |
| 13621 | aagacagcta attgaggcta aatgggtctg atgccattct attagtgcaa aataaattgg |
| 13681 | cttatgccaa cttgaactcc taatcttgat tttatcaaca ctttgccctg tttattcaga |
| 13741 | ctgttcatga gtaggaagtt aaggaaaatg ttgcctatga acaaaatgat caaactttta |
| 13801 | tctgaaatac ctttgaaagt tttcccgaag ctttaattat catgttcctt ttgttttgga |
| 13861 | tgtaatagca ggccctccca attttcctaa atgtttgaaa atatttttga tactttctgt |
| 13921 | cttctgttgt ggttaaaaaa aagaaggcac cagtgttggc tatgcctggg gagttagtga |
| 13981 | ggtaatctgg agagatctct ggaccccagt cctttgtcca agaggtatgg tcaaaagaac |
| 14041 | aaagcaaatt tcagaaaaac cggtataata tgatcctatt ttaatggtaa gtctgttcca |
| 14101 | acaactgctt tctttttcga attacaccat gtgcccaggc tacaaaggct ctgcagaaat |
| 14161 | gtgactctgt tggcaaatac gcgtgtgata ccttaaccat gctggaggcc agttagcata |
| 14221 | atgbitattt cagatgcagt ttgtcatcag tgcbgggtgc tatggggctt aaaacaaaaa |
| 14281 | caggatgagc ctagagcaca gaagtggaaa attaggtggg tgggtgagta tggacttgga |
| 14341 | aatggaaaat gtgcttccct gtgggacatg gcttgtgttc attagtgctg acctgcctcc |
| 14401 | tctctagccc tgtaagctga cgctgacacc aaagcttgct gagaaactga atagaccctc |
| 14461 | ccgccccgac caaccaagga caaagatgag accacccaat tcggttacgt tgttgccaga |
| 14521 | acaacatgtt taatctttaa catgggttca aactattctt ggccctagga acatggactt |
| 14581 | cagcaattaa agtcaacatg tgcttccatt aaaccattgt gtccacctcc aaatggttat |
| 14641 | aaatatgtag cataagctat gggagttggg gtggaatgga tgacagcctt tgctaatgac |
| 14701 | gctctagaaa ccaatattct cctctcaaaa taacatggaa aaattctact taatacctcc |
| 14761 | tgggttccat tagtggttct gggtaaataa ttctggggaa agtaatttct gttttcattc |
| 14821 | cagctgtgca gcagatactg tgatgatgga ttgcaagtgc aaagagtaag acaaaactcc |
| 14881 | agcacataaa ggacaatgac aaccagaaag cttcagcccg atcctgccct ttccttgaac |
| 14941 | gggactggat cctaggaggt gaagccattt ccaatttttt gtcctctgcc tccctctgct |
| 15001 | gttcttctag agaagttttt ccttacaaca atgagaaaac atgtactagc tgcatccttt |
| 15061 | tctatgctct ccctgcbggt gataatggga gatacagaca gtaaaacgga cagctcattc |
| 15121 | ataatggact cggaccctcg acgctgcatg aggcaccact atgtggattc tatcagtcac |
| 15181 | ccattgtaca agtgtagctc aaaggtaaga ccaaggtctc tgtgaggaga gcatttccct |
| 15241 | cagaaacagg caagaagcca tatttctttg tcaggggatg gagaactaag atcatagccc |
| 15301 | aaacttaagt gatgtggctg gcccacaggg cttgtgggag aatgaggctt ttagaatttc |
| 15361 | cccccgtgct ttgcactgtt atcagtaaaa gtgcctaatc aatcatgtgc acacccatcc |
| 15421 | agtgggacag gtgtcctttg tccctggctg tgatgtttat gagtggtagt gtgcttttcc |
| 15481 | aaaaaagaaa aatggagttt acaagtcatc ataactctct agctccatca tcaataaaga |
| 15541 | ggaagagaat gcaaagaatg cgattgagca gtggaacata cttcttggag ggctctttaa |
| 15601 | agagattctt ggtgaaataa ctggctttgt ggtctgagcc tttcttcagg tgctacattt |
| 15661 | ctgttcaaat gatttcaaaa gataagatgg caatatgatc atttatcatt tttacattgc |
| 15721 | ttatccaaat cgttctcatt tggaaaagga tgcabitttc ttgcagaggc agtccgtgcc |
| 15781 | tcagtggtgt gcaagggaat atcatgctta aaaggccacc aagagtggcc agctacatgg |
| 15841 | accacccaga agggaggcac attgtagggt ggaacatacg agatctcacg actgtcactg |
| 15901 | cccctaaaca ggcacaatga atgaaatttg tccacacttg gtaaataata atttgtttag |
| 15961 | cccaacctca gagattttta catttagttt tgagatgaaa atcacttaaa cattctcgta |
| 16021 | tggcaagtaa tttttgaaat taccagtata attaagaatt ttttatagta tcaatcacca |
| 16081 | gaattaagac catataagcc aatccctcca ctctccattc aaaaacatta tgacttttgt |
| 16141 | atgccttgaa aatttccatt aatttctctg attatagctc agtgatgatg attcatttta |
| 16201 | gtttaggagg atcagaattc gagttaaatc attttaacca ttatattaat aagatctatg |
| 16261 | actgbjggaa aaaatagaga ttttccactt aataaaaact ttctggctct tatttgtgct |
| 16321 | tgtagaacca aaaaaaaatg gagtaaatag atgaacataa atttttgcag ggccacaatt |
| 16381 | ccttcttagg aaagggaaac taaaacaaat tgagaaaaaa catgagaagt gcttttacaa |
| 16441 | gtgtgattgc tcagaaattg tgggcccatg taaggttgac tgcagcaatt attagtttct |
| 16501 | ccttccctcc cccttcagaa acagacagca tgaatattta gtgattttat tccatggata |
| 16561 | atttctttcc gtattgagtt gtcctccata atcgtgattc ttttttaaaa aaaggaatta |
| 16621 | aaattatttc aaagaaagtg tcactatgat gatagaaagg aacaaagaaa taggagcatc |
| 16681 | tataaaaact agaaaacaat ctggaaagac tggacaaaat gatggaggag tactttggca |
| 16741 | acaatcacat taatctagcg aaggtcaaag aagagacatc atgaactcaa accgaagaaa |
| 16801 | ttcacatgtg aattctgagc atgagtctta gaaacagact ttagtgtact gttattcacc |
| 16861 | tttgtttctt ccagaaagcc atggcaaata acagcgcaaa attgtgatag caggagaaat |
| 16921 | gtttgtaatt tcatgggaac aatctgtttt caaacatccc aaactatccc aaatatagca |
| 16981 | atagttttat gtaaatggtg cttgcaatta taaacctttc ttccctggcc ataaaatatg |
| 17041 | cctggcaaaa tctcaactac ccagaccttt tctaggttag atttttctct atgtatccag |
| 17101 | gagcaaaact gttttcattt ggaaatgttc tataattctt aactcttaca ttctcgggct |
| 17161 | gatcttttct ctaatgggtc tcacttaatg aaattgtttg cctttagaga cttggataaa |
| 17221 | acatttagcc tatgtctaga aaagaggcaa caacaatcta ccctagcttc tttttacaaa |
| 17281 | gaaaagactg ggatagattt ttttgtctct ataaaaatta tcccttgaca tcatgttcaa |
| 17341 | gggtcattat ttattaaaca tgtgcctact gtttgcctgc caattgttaa tgctttctga |
| 17401 | ctcacattta tccattatct ggccttgtaa aggcaagaaa gggagaatgt gacccaggct |
| 17461 | cagggaaggg agtgtacctg cttgaacacc taatatatgt gtcaggcacc tcatattgta |
| 17521 | tattataatc attgccaaga aacctgtgat ggcaggcatt attattccca ttatcctggt |
| 17581 | aaggaaaact gaagcccaaa ataattagat tgcttgatat catatagcca gggagtggca |
| 17641 | atgtctgcct tacagcattc atcttcccac tacttggatc ttcatattgg aggaacacaa |
| 17701 | gtagaatgta ggagccatca catagaagtt caggctctgc ttctgatctg ctggcagtaa |
| 17761 | taatttatac tcagcaatgt ttctagaaaa aataaataat agtgaaagcc tttctgggag |
| 17821 | ctttcaatga gactgttcat cccagccctg aacttgcagc tggccagtcc taggtccctg |
| 17881 | ctgcccatct cctagcaatt ccggaaagga ctgagaggga gtcaggcaga aaagactggc |
| 17941 | cacagtggcc caccaaagag gacagtgtgg ccacagatta ggatgtgcct gatagtttta |
| 18001 | agtggtggca cctcctgggt ttgggtacaa aagatcctgg gagtagcagg ttcatttata |
| 18061 | atcttcatgg aagactaagt ttctctaaaa tttggagtaa aggctgtaga gcaaacaggc |
| 18121 | aaaagaccag ttgatagtgg aaacagactg cctggtggaa tctctatgag aactgaggca |
| 18181 | tgccaggctt tgaagtgaga gagacctggt ttccaattcc agctccatgg cttgccgaat |
| 18241 | gagtgacttt gaggaagttc agtaacttcc ttctttgtgc ctcagtttct tcccatgcaa |
| 18301 | aatagagctg tctacctgag ggtatgggta tgggtgttgg tgatttgtaa agcatccagc |
| 18361 | ggtgtccagc acataatagc catttaatag aagatattat gatcttgatg ataaagttat |
| 18421 | aaactaagca ggtgtttaga ctcaattttc tatttcagtc acaactgaga tctcaataac |
| 18481 | tgctgggatc tcctatagcc ttaaaagatt aggaaaatga accttattgt agttcttgtt |
| 18541 | aagcacaggg tggggcagaa gagcaaagta aatgtaagcc actgtgctgg gtgctaacag |
| 18601 | ggaggataaa agtaaaagag aaacagcccc gatatctgtc taaccttcct tcccagatga |
| 18661 | gtggaggaag gaggggctgg agggggatga gttttggact tgggccaggg aggggatttc |
| 18721 | aggaatggag ggtgggaaag caaactggca gggaagacct ggaaagtctc agttgggtga |
| 18781 | ggagtggggg atgctggccc caggccaggg catggaacca gggcatgact atctgaaccg |
| 18841 | gtaagggcca caggagcagg tggggaactg gaagagcaac aacaaaggat aaggaggaag |
| 18901 | aggtttggga gacaaaacct aggaaaagag gacctggtat gctaagggct gagtcctaac |
| 18961 | agcttcctaa caggaccccc aagccatctt ctactgctac ccaccaagct catggtgtga |
| 19021 | taggagaaat ggagggagtc aggaaaattg tgtttataat acaatagact tcttgagaga |
| 19081 | tataatttga tttttttaaa aggaacaaat ttatactgag tgcctaccat gtccaggtgc |
| 19141 | tgtggaaggt gctggagact ctaaagtgga aagaaagaaa aaagagacaa tttttttgcc |
| 19201 | ttcatgatgc ttacaagaaa atggggagag agggaggagg ggtgttctgt gcacaaagat |
| 19261 | cagtttgtat ctgtttgatt catgaccgga gtgaagggac cagtttctag ttggagtgag |
| 19321 | ctgagttccc atccaccctt tcaaagcccc caccatctct ctgctgcttt gttccctggt |
| 19381 | tgcaattttc ttggtgccct cagcctagca cagatctttt ggaggttgac ctgggtccct |
| 19441 | gaagatgagc ggagttatga ggctgcccgt ggggaaagaa tgggagaaga agcatgggtg |
| 19501 | aatcttgttc ctgaggttat agagggtgtt tcaagaggtg ccatggaaat aagggtgcat |
| 19561 | agtgttcact gctgcgaaaa aggcaaaata cttttccact cbitcggagt aaaatcgcaa |
| 19621 | aaatgctctt ctagggtggg acaggtgggt ctttattctg atgctaatgg tgacatctgg |
| 19681 | tcagtcaagg ctcttggaag attcagctct gcataagctt ggggatggtg aagctaatgt |
| 19741 | cccaagaagc ccacaagaaa aagttagact tgtatgtcta tggatatggt caggggtttc |
| 19801 | tgaggcaaat caatgtagat aaggaagtct gtgagttggc atatgcttga ggaaaacatg |
| 19861 | gtatcaggag gcaggtatta tactggttta ggtcagggct ttggtttcag atagaccttg |
| 19921 | gtttgattcc agctctgcat tttaccacct atgtgagttt gggcaagtta ctggaactct |
| 19981 | ctaaagcttg agtttcctta tctgtcctaa tagtgtagat cataacagta ctttcttgta |
| 20041 | gggttgttgt aaggatgaaa taactcacat tgcaagtgct caataaatgt tcttattagc |
| 20101 | gaaatgatgg ttatctagtg attcatctag agcacagccc tcaaacttga atgggtccag |
| 20161 | agacctgcta ctcaaagtgt gggcaccagc ccagtaggac tgacatcaac tggaatctga |
| 20221 | tgggaaatgc agaatctcag gctctaggcc agaactactc aatcagaact tgccttttaa |
| 20281 | caagatccta gatgattcac acgcacattc cagtctgaga agctctgatc cggaagagat |
| 20341 | caggagatgt tcaagtgctg aaacacaagt aaggcatggc agaagctaaa atgtgaagta |
| 20401 | ttgagagtaa accaggagag cagatggtta ttccttccat cccttaagga tcttatcttt |
| 20461 | caaacttctg ctttaccact tcaacacaca aaacagagat agatttcctt ctaggagtgt |
| 20521 | gtgtgtgtgt gtgtgtgtgt gtgtgtgttg agaacatcaa tggttcaata attgcttcat |
| 20581 | agtcttttct tccaggaaag agcatttgaa tttggctaca gagaatagat aggtgggatt |
| 20641 | ttattaggca gagcaaggtg gaaaggacat atttgggaga cagaagacat aagcaaaggc |
| 20701 | aagaggcagg agaattcagg gcactttctt ggactcctaa gtagttaagt ttgactggag |
| 20761 | tagggagtgg gtagtagagt gatgagaata gttgtaagta agactggaaa tacatgtgaa |
| 20821 | acctgggtca tgaagggcct tgactcctct tcaagatttt ggattttatt ctgaactact |
| 20881 | gaaggcattt attgtacatg tctgtgcatt cttcttgcac aaacagtcca gctcccaagg |
| 20941 | aagaggggcc cagcccattg gaggtggccc cacgtccttc agtgcctgga gttcttgtcc |
| 21001 | ttcagagtgg gcbgggggtg gagctggaca tcctgaaaac ttttagtgag attgcaatca |
| 21061 | gatgagctca tttacaaatg gccctacgct gttgacttgg tcattttatg gacagtgcta |
| 21121 | atggcccagt cagttaggcc tacgtttggt tcacctgtgt atacatcact tggatattat |
| 21181 | gtcccatatc attgttggcc cttctgagtt aagttcgttg gcacagattt actttgtcca |
| 21241 | cccagactga aactgcacct ctgaagactc catcacggaa ggtgactctg tgaataactg |
| 21301 | cttagtcaac ctaatagatg atctagatga gagattagca aatttaacac tactccaagg |
| 21361 | tgaggtatca agcagctctt cccacataca gggagatggg gggagaacag actaaggaag |
| 21421 | atcttggact ctctctctca ttagagaaat gggggtggga attagggact agcctgtgta |
| 21481 | ctactgacct atggcatagt ctctcaaaac ccatttcaga gccaggcaca gtggtacatg |
| 21541 | cctgtagtcc tggctacaca ggaggctgag gcaggaggat cccttgagct caggagttta |
| 21601 | aggctgtagt gtgctatgat catgcctgtg aataaccact gcactgcagc ctggacaaca |
| 21661 | tagcaagact ctgtctctaa aaacaaaaca aaacaaacaa acaatcattt ctgatagttt |
| 21721 | actttgcctg tcaaaatgaa gagcaggtag gatagaatca aaagctttaa aaaaatcaaa |
| 21781 | acagcttaca gctgctattt tctctctgcc tctgcgatca ttcagagaaa ataaattcac |
| 21841 | tggtctagtc caacttattc ttccctaagc cctgctgttt tttttttcta gtatttcata |
| 21901 | ttgccttggt ggttgctaag tgattgttgg atttagcccc agtatattct cagataacta |
| 21961 | tgccaataat ctataattac cagaattcat cttttctccc ttttggaaag atggactggc |
| 22021 | tatttttttc ttgtaaacct tgggtgttta attctgccct acaggatgat gtcagagata |
| 22081 | atagctggtg agtctttaat aataactgcg aattcctaca atattgtaag gtaaacgtca |
| 22141 | aaaggtttca tgcatatata tgtatctttt aatctttcat ttgaacagtt tcacttttta |
| 22201 | tgatcttgct gttggttctg gaattatagg acaaattttt cttttctttt cttttctttt |
| 22261 | tgagacaggg ccttgctctg tcactcatgc tggagtacag tggcttgatc atagcttcct |
| 22321 | gcagcctcta attcctgggt tcaagtgatc ctcctgcctc agctgccaaa gtagctggga |
| 22381 | ctacaggtgc acatcaccac gcctggttaa ctttttattt ttttttttag agacacggtc |
| 22441 | atgctatgtt gcccaggctg gtctcaaact cctggggtca agtgatcctc ctgcctcaga |
| 22501 | ctccaaagtg ttgggattat aggcatgaga caatacacct ggcccagggg atttttttct |
| 22561 | ccatcactta acatgaaatt caccaagcaa aacctcttac ataaccatgg gaccaataat |
| 22621 | ctcagattct ccattcccct ccccaccttg atattaggat tatgcagctt aatgagtcaa |
| 22681 | gctgtcccac cctacccacc tattactccc actcccagga taccaggagt tttgtatggg |
| 22741 | atacataaat gatccttttt tccattccgt taagctgtat gcaaattgtg tgtgtgctta |
| 22801 | tgtacatttt tctggggata actttggtag ttttcatcag attcttaaag gtgtctgttt |
| 22861 | gctaaagcca gattaagagc tactataaag aacacatagc ctccgttatt ccagcccaat |
| 22921 | ctttggaaat agagattatc agaatgggta catcctaata tttgccgcaa atgactctga |
| 22981 | agatgcggat gcaattacat aaatcccagg ctccttcaac atataaaccc agaagtgtga |
| 23041 | tttcagggcc ccagacatgc cctgtctcta cttcattctc ttgtcccaaa ttgcagcacc |
| 23101 | cagaatattt cttcctaagc attactcagg ctatcctatt ttcttattca gaaactatat |
| 23161 | tcctgttctt tagctatttc ctgcacaaat caatttcttt tttttttttt tttttttggt |
| 23221 | agtcatttta atttacttct tggtttcaat ttggattgac tttctgcatt ttgctactgg |
| 23281 | tagaattgca atgcattggg ccagtggtag accactacct gtcaggagaa atttcttttg |
| 23341 | ttctttggtt tttactcgca catcacagtg atgggcgtca ttcagtggtt ctaaaccctc |
| 23401 | agtacactta gaaatcacct aagaagcttt gaaaaccatg aaagcctggt ccctatctcc |
| 23461 | aaggatttta attcaatcga tctgaatgag gcctgggtgt tgacttaaaa aaaatgcaac |
| 23521 | atgcattgag ccactggtct aatctagcag gaattggcaa cgagtgtgag ggtctttcaa |
| 23581 | aagatgctgt tttacctggc taaggttgtg gcatgcccac agagtaacca cctccttttc |
| 23641 | tttccccaga tggtgctcct ggccaggtgc gaggggcact gcagccaggc gtcacgctcc |
| 23701 | gagcctttgg tgtcgttcag cactgtcctc aagcaaccct tccgttcctc ctgtcactgc |
| 23761 | tgccggcccc agacttccaa gctgaaggca ctgcggctgc gatgctcagg gggcatgcga |
| 23821 | ctcactgcca cctaccggta catcctctcc tgtcactgcg aggaatgcaa ttcctgaggc |
| 23881 | ccgctgctgt gtgtggcttc tggatgggac aactgtagag gcagttcgac cagccaggga |
| 23941 | aagactggca agaaaagagt taaggcaaaa aaggatgcaa caattctccc gggactctgc |
| 24001 | atattctagt aataaagact ctacatgctt gttgacagag agagatactc tgggaacttc |
| 24061 | tttgcagttc ccatctcctt tctctggtac aatttctttt ggttcatttt cagattcagg |
| 24121 | cattttcccc cttggctctc aatgctgttt gggtttccaa caattcagca ttagtgggaa |
| 24181 | aaagtgggcc ctcatacaca agcgtgtcag gctgtcagtg tttggtgcac gctggggaag |
| 24241 | aatttacttt ggaaagtaga aaagcccagc ttttcctggg acatcttctg ttattgttga |
| 24301 | tgtttttttt taccttgtca ttttggtcta aggttgccat tgctgctaaa ggttaccgat |
| 24361 | ttcaaagtcc agataccaag catgtggata tgtttagcta cgtttactca cagccagcga |
| 24421 | actgacatta aaataactaa caaacagatt cttttatgtg atgctggaac tcttgacagc |
| 24481 | tataattatt attcagaaat gactttttga aagtaaaagc agcataaaga atttgtcaca |
| 24541 | ggaaggctgt ctcagataaa ttatggtaaa attttgtaag ggagcagact tttaaagact |
| 24601 | tgcacaaata cggatcctgc actgactctg gaaaaggcat atatgtacta gtggcatgga |
| 24661 | gaatgcacca tactcatgca tgcaaattag acaaccaagt atgaatctat ttgtgggtgt |
| 24721 | gctatagctt tagccgtgtc acgggcatca ttctctaata tccacttgtc catgtgaaac |
| 24781 | atgttgccaa aatggtggcc tggcttgtct tctgaacgtt tggttcaaat gtgttttggt |
| 24841 | cctggaggct caaattttga gttattccca cgttttgaaa taaaaagagt atattcaaaa |

Mouse Mature NDP Protein (NCBI Accession No. NP_035013.1)
(SEQ ID NO: 23)
KTDSSFLMDSQRCMRHHYVDSISHPLYKCSSKMVLLARCEGHCSQASRSEPLVSFSTVLKQPERSSCHCCR
PQTSKLKALRLRCSGGMRLTATYRYILSCHCEECSS
Mouse Mature NDP cDNA
(SEQ ID NO: 24)
aaaacagacagttcatttctgatggactctcaacgctgcatgagacaccattatgtcgattctatcagtca
cccactgtacaaatgtagctcaaagatggtgctcctggccagatgtgaggggcactgcagccaggcatcac
gctctgagcccttggtgtccttcagcactgtcctcaagcaacctttccgttcctcctgtcactgctgccga
ccccagacttccaagctgaaggctctgcgtctgcgctgctcagggggcatgcgacttactgccacttaccg
gtacatcctctcctgtcactgtgaggaatgcagctcc
Rhesus Monkey Mature NDP Protein (NCBI Accession No. NP_001253901.1)
(SEQ ID NO: 25)
MRKHVLAASFSMLSLLVIMGDTDSKTDSSFIMDSDPRRCMRHHYVDSISHPLYKCSSKMVLLARCEGHCSQ
ASRSEPLVSFSTVLKQPFRSSCHCCRPQTSKLKALRLRCSGGMRLTATYRYILSCHCEECNS
Rat Mature NDP Protein (NCBI Accession No. NP_001102284.1)
(SEQ ID NO: 26)
KTDSSFLMDSQRCMRHHYVDSISHPLYKCSSKMVLLARCEGHCSQASRSEPLVSFSTVLKQPFRSSCHCCR
PQTSKLKALRLRCSGGMRLTATYRYILSCHCEECSS
Chimpanzee NDP Protein (NCBI Accession No. XP_016799622.1)
(SEQ ID NO: 27)
KTDSSFVMDSDPRRCMRHHYVDSISHPLYKCSSKMVLLARCEGHCSQASRSEPLVSFSTVLKQPFRSSCHC
CRPQTSKLKALRLRCSGGMRLTATYRYILSCHCEECNS

### Trio Rho Guanine Nucleotide Exchange Factor (TRIO) and F-Actin Binding Protein (TRIOBP)

The TRIOBP gene encodes “trio Rho guanine nucleotide exchange factor (TRIO) and F-actin binding protein” (TRIOBP), a protein that is expressed along the length of the stereocilia actin filaments and in the stereocilia rootlets of the inner hair cell. TRIOBP is important for the stability and rigidity of the stereocilia of specialized hair cells in the inner ear (see, e.g., Kitajiri et al. (2010) Cell 141: 786-798; and Zhao et al. (2015) Curr. Opin. Neurobiol. 34: 172-179).

Stereocilia, which are about 10-50 μm in length, are important for hearing and balance. Stereocilia play a mechanosensing role in hearing. By bending in response to sound, they form a structure for mechanoreception of sound stimulation.

The human TRIOBP gene is located on chromosome 22q13.1. It contains 24 exons encompassing ˜80 kilobases (kb) (Hirosawa et al. (2001) DNA Res 8:1-9; NCBI Accession No. NG_012857.1). To date, there are three known families of TRIOBP splice isoforms: TRIOBP-5, TRIOBP-4 and TRIOBP-1 (see, e.g., Kitajiri et al. (2010) Cell 141: 786-798). While TRIOBP-1 is ubiquitously expressed, TRIOBP-5 and TRIOBP-4 are expressed predominantly in the inner ear and in the eye. TRIOBP-5 gene (NCBI Accession No. NM_001039141), the longest isoform, contains 24 exons, and the translation start site for the TRIOBP-5 isoform is found in exon 3. The TRIOBP-5 protein has a molecular mass of ˜218 kDa. The TRIOBP-4 is gene encoded by exons 1-6 and is expressed from the same promoter as TRIOBP-5. The TRIOBP-4 protein has a molecular mass of ˜107 kDa. TRIOBP-1 (NCB1 Accession No. NM-007032) is encoded by exons 11a-24, has a molecular mass of ˜72 kDa, and is translated from a start site in exon 11a (see, e.g., Siepel et al. (2001) J. Cell. Sci. 114: 389-399).

Various mutations in the TRIOBP genes have been associated with non-syndromic hearing loss (e.g., profound, prelingual deadness DFNB28 (MIM #609761) (see, e.g., Riazuddin et al. (2006) Am J Hum Genet 78: 137-143) and age-related hearing impairment (see, e.g., Hoffmann et al. (2016) PLoS Genet 12(10): e1006371)). DFNB28-causing mutations have been predominantly found in exon 6 of TRIOBP, an exon that is present in both TRIOBP-4 mRNA and TRIOBP-5 mRNA. DFNB28-deafness can be modeled by generating TRIOBP-4/5 deficient mice, in which mouse exon 8, the ortholog of exon 6 of human TRIOBP-4 and TRIOBP-5, is mutated/deleted (see, e.g., Kitajiri et al. (2010) Cell 141: 786-798). Nonsense mutation (c.5014G>T (p.Gly1672*) in exon 9 of TRIOBP-5) and frameshift mutations (c.2653del (p.Arg885Alafs*120), c.3460 3461del (p.Leu1154Alafs*29), and c.3232dup (p.Arg1078Profs*6) in exon 6)) were found in Dutch families with congenital and moderate hearing impairment (see, e.g., Wesdorp et al. (2017) Hearing Research 347: 56-62). The nonsense mutation found in exon 9 was the first disease-associated mutation that was present only in TRIOBP-5.

Additional exemplary mutations in a TRIOBP gene that have been detected in subjects having hearing loss and methods of sequencing a nucleic acid encoding TRIOBP are described in, e.g., Shahin et al. (2006) Am J Hum Genet 78:144-152; Diaz-Horta et al. (2012) PLoS One 7: e50628; Gu et al. (2005) Clin. Genet. 87: 588-593; and Table 1 in Wesdorp et al. (2017) Hearing Research 347: 56-62. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype TRIOBP protein is or includes the sequence of SEQ ID NO: 28, SEQ ID NO: 30, or SEQ ID NO: 31, SEQ ID NO: 32. Non-limiting examples of nucleotide sequences encoding a wildtype TRIOBP protein are or include SEQ ID NO: 29, and SEQ ID NO: 33.

In some embodiments of any of the compositions described herein, the TRIOBP protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 28.

In some embodiments of any of the compositions described herein, the TRIOBP protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 30.

In some embodiments of any of the compositions described herein, the TRIOBP protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 31.

In some embodiments of any of the compositions described herein, the TRIOBP protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 32.

Human Full-length Wildtype TRIOBP Protein Isoform 5
(SEQ ID NO: 28)
MEEVPGDALCEHFEANILTQNRCQNCFHPEEAHGARYQELRSPSGAEVPYCDLPRCPPAPEDPLSASTSGC
QSVVDPGLRPGPKRGPSPSAGLPEEGPTAAPRSRSRELEAVPYLEGLTTSLCGSCNEDPGSDPTSSPDSAT
PDDTSNSSSVDWDTVERQEEEAPSWDELAVMIPRRPREGPRADSSQRAPSLLTRSPVGGDAAGQKKEDTGG
GGRSAGQHWARLRGESGLSLERHRSTLTQASSMTPHSGPRSTTSQASPAQRDTAQAASTREIPRASSPHRI
TQRDTSRASSTQQEISRASSTQQETSRASSTQEDTPRASSTQEDTPRASSTQWNTPRASSPSRSTQLDNPR
TSSTQQDNPQTSFPTCTPQRENPRTPCVQQDDPRASSPNRTTQRENSRTSCAQRDNPKASRTSSPNRATRD
NPRTSCAQRDNPRASSPSRATRDNPTTSCAQRDNPRASRTSSPNRATRDNPRTSCAQRDNPRASSPSRATR
DNPTTSCAQRDNPRASRTSSPNRATRDNPRTSCAQRDNPRASSPNRAARDNPTTSCAQRDNPRASRTSSPN
RATRDNPRTSCAQRDNPRASSPNRATRDNPTTSCAQRDNPRASRTSSPNRATRDNPRTSCAQRDNPRASSP
NRTTQQDSPRTSCARRDDPRASSPNRTIQQENPRTSCALRDNPRASSPSRTIQQENPRTSCAQADDPRASS
PNRTTQQENPRTSCARRDNPRASSRNRTIQRDNPRTSCAQRDNPRASSPNRTIQQENLRTSCTRQDNPRTS
SPNRATRDNPRTSCAQRDNLRASSPIRATQQDNPRTCIQQNIPRSSSTQQDNPKTSCTKRDNLRPTCTQAD
RTQSFSFQRDNPGTSSSQCCTQKENLRPSSPHRSTQWNNPRNSSPHRTNKDIPWASFPLAPTQSDGPRTSS
PSRSKQSEVPWASIALRPTQGDRPQTSSPSRPAQHDPPQSSFGPTQYNLPSRATSSSHNPGHQSTSRTSSP
VYPAAYGAPLTSPEPSQPPCAVCIGHRDAPRASSPPRYLQHDPFPFFPEPRAPESEPPHHEPPYIPPAVCI
GHRDAPRASSPPRHTQFDPFPFLPDTSDAEHQCQSPQHEPLQLPAPVCIGYRDAPRASSPPRQAPEPSLLF
QDLPRASTESLVPSMDSLHECPHIPTPVCIGHRDAPSFSSPPRQAPEPSLFFQDPPGTSMESLAPSTDSLH
GSPVLIPQVCIGHRDAPRASSPPRHPPSDLAFLAPSPSPGSSGGSRGSAPPGETRHNLEREEYTVLADLPP
PRRLAQRQPGPQAQCSSGGRTHSPGRAEVERLFGQERRKSEAAGAFQAQDEGRSQQPSQGQSQLLARQSSP
APSRQVTMLPAKQAELTRRSQAEPPHPWSPEKRPEGDRQLQGSPLPPRTSARTPERELRTQAPLESGQAGP
RQPLGVWQSQEEPPGSQGPHRHLERSWSSQEGGLGPGGWWGCGEPSLGAAKAPEGAWGGTSREYKESWGQP
EAWEEKPTHELPRELGKRSPLTSPPENWGGPAESSQSWHSGTPTAVGWGAEGACPYPRGSERRPELDWRDL
LGLLRAPGEGVWARVPSLDWEGLLELLQARLPRKDPAGHRDDLARALGPELGPPGTNDVPEQESHSQPEGW
AEATPVNGHSPALQSQSPVQLPSPACTSTQWPKIKVTRGPATATLAGLEQTGPLGSRSTAKGPSLPELQFQ
PEEPEESEPSRGQDPLTDQKQADSADKRPAEGKAGSPLKGRLVTSWRMPGDRPTLFNPFLLSLGVLAWARP
DLLNFKKGWMSILDEPGEPPSPSLTTTSTSQWKKHWFVLTDSSLKYYRDSTAEEADELDGEIDLRSCTDVT
EYAVQRNYGFQIHTKDAVYTLSAMTSGIRRNWIEALRKTVRPTSAPDVTKLSDSNKENALHSYSTQKGPLK
AGEQRAGSEVISRGGPRKADGQRQALDYVELSPLTQASPQRARTPARTPDRLAKQEELERDLAQRSEERRK
WFEATDSRTPEVPAGEGPRRGLGAPLTEDQQNRLSEEIEKKWQELEKLPLRENKRVPLTALLNQSRGERRG
PPSDGHEALEKEVQALRAQLEAWRLQGEAPQSALRSQEDGHIPPGYISQEACERSLAEMESSHQQVMEELQ
RHHERELQRLQQEKEWLLAEETAATASAIEAMKKAYQEELSRELSKTRSLQQGPDGLRKQHQSDVEALKRE
LQVLSEQYSQKCLEIGALMRQAEEREHTLRRCQQEGQELLRHNQELHGRLSEEIDQLRGFIASQGMGNGCG
RSNERSSCELEVLLRVKENELQYLKKEVQCLRDELQMMQKDKRFTSGKYQDVYVELSHIKTRSEREIEQLK
EHLRLAMAALQEKESMRNSLAE
Human Wildtype TRIODE Isoform 5 cDNA
(SEQ ID NO: 29)
atggaggag gtgcctgggg atgccctgtg tgaacacttt gaggccaaca tacttaccca
gaaccgctgt caaaactgct tccaccctga ggaggcccat ggagcaagat accaggagct
caggagccct tcaggtgctg aggtgcccta ctgcgacctg cctcgatgtc cacctgcccc
tgaggaccca ctcagcgcct caacctccgg ctgccagtct gtggtggacc caggcctcag
gccagggccc aagaggggcc catccccctc agcagggctc ccagaagagg gtcccacagc
tgcccccagg agcaggagcc gggagcttga ggcagtaccc tatctggagg gcctgaccac
ttccttgtgt ggcagctgca acgaggaccc cggctctgac cccacctcca gccctgactc
cgccacccct gatgatacca gcaactcgtc ctctgtggac tgggacactg ttgagaggca
ggaggaggag gcccccagct gggacgagct cgcagtgatg atcccgagga ggcctcggga
ggggccgaga gctgacagct cccaaagggc tccgtctctc ctcaccaggt cccctgtggg
aggagatgct gcaggccaga aaaaggagga caccggcggt gggggccgga gcgcaggaca
gcactgggca aggctccggg gagaaagcgg gttgtccctg gagcggcacc ggtcaacact
gacccaggct tcctccatga caccacacag tggacctcga agcaccacgt ctcaggcttc
tcctgcccaa agggacactg ctcaggctgc ctctacacgt gaaatcccca gagcctcctc
tccccatcga atcacccaaa gggacacctc cagggcctca tccacccaac aggaaatctc
cagggcctca tccacccaac aggaaacctc cagggcctca tccacccaag aggacacccc
tagggcctca tccacccaag aggacacccc cagggcctca tctacacagt ggaacacccc
cagagcttcc tctccctcac gaagcaccca actggataac cccagaacct cttctaccca
gcaggacaac ccccaaactt cttttcctac ttgtactccc cagcgggaaa accccaggac
accctgtgtc cagcaggacg atcccagagc ctcctctccc aacagaacca ctcaacgaga
gaattccaga acatcctgtg cccagcggga caatcccaaa gcctccagaa cctcctctcc
caatagagcc acacgagaca accccagaac atcctgcgcc cagcgggaca atcccagagc
ctcctctccc agtagagcta cacgagacaa ccccacaaca tcctgtgccc agcgggacaa
tcccagagcc tccagaacct cctctcccaa tagagccaca cgagacaacc ccagaacatc
ctgtgcccag cgggacaatc ccagagcctc ctctcccagt agagctacac gagacaaccc
cacaacatcc tgtgcccagc gggacaatcc cagagcctcc agaacctcct ctcccaatag
agccacacga gacaacccca gaacatcctg cgcccagcgg gacaatccca gagcctcctc
tcccaataga gctgcacgag acaaccccac aacatcctgt gcccagcggg acaatcccag
agcctccaga acctcctctc ccaatagagc cacacgagac aaccccagaa catcctgtgc
ccagcgggac aatcccagag cctcctctcc caatagagct acacgagaca accccacaac
atcctgtgcc cagcgggaca atcccagagc ctccagaacc tcctctccca atagagccac
acgagataac cccagaacat cctgtgccca gcgggacaat cccagagcct cctctcccaa
cagaaccacc caacaagaca gccccagaac atcctgtgcc cgacgggacg atcccagagc
ctcctctcct aacagaacca tccaacaaga gaaccccaga acatcctgtg ccctacggga
caatcccaga gcctcctctc ccagcagaac catccaacaa gagaacccca gaacatcctg
tgcccaacgg gacgatccca gagcctcctc tcctaacaga accacccaac aagagaaccc
cagaacatcc tgtgcccgac gggacaatcc cagagcctcc tctcgcaaca gaaccatcca
gcgagacaac cccagaacat cctgtgccca gcgggacaat cccagagcct cctctcctaa
cagaaccatc caacaagaga acctcagaac atcctgtacc cgacaggaca atcccaggac
ctcctctccc aatagagcca cacgagacaa ccccagaaca tcctgtgccc agcgggacaa
tctcagagcc tcctctccca tcagagccac ccaacaggac aaccccagaa cttgtattca
acagaacatc cccagatcat cttctaccca acaagacaac cctaaaacct cttgtaccaa
acgagataac ctcagaccca cttgtacaca gcgggaccgc acacagtcct tttcctttca
acgagacaac cctggaacct cctcatctca atgctgcacc caaaaggaga atctgagacc
atcatctccc caccgctcca ctcaatggaa caatcccagg aattcatctc cccatcgtac
taacaaagac atcccctggg cctcgtttcc cctccggcca actcagagtg atggtccccg
aacctcttcc ccatctcgct ccaagcaaag cgaggttccc tgggcatcca tcgccctccg
gccaacccaa ggtgacaggc ctcagacatc ctctcccagc aggccagccc agcatgaccc
accccagtcc tcctttggcc ccacccagta caacttgcca tcccgggcca cctcttcctc
ccataaccca ggccaccaga gcacctcccg aacttcctca cctgtgtacc ccgctgccta
tggggctccc ctgacctctc ctgagccctc ccagcctcca tgtgctgtgt gcattgggca
ccgggatgcc cctcgagcct cttcgccccc tcgctatttg cagcacgacc ccttcccctt
cttcccagag ccccgcgccc ctgagagtga accgccccac cacgagcctc cctatatacc
acctgctgtg tgcattggac accgagatgc cccccgggcg tcctcgcccc cccgccacac
ccaatttgac cccttcccct tcctcccaga cacatcagat gccgagcatc agtgtcagtc
cccccaacac gagccccttc agctccctgc acctgtgtgt attgggtacc gagatgcacc
ccgggcctcc tccccaccac gccaggcccc agagccttcc ctcttattcc aggacctccc
cagggccagc acagagagcc ttgtcccttc catggactct ctgcacgagt gcccccacat
ccccacccct gtgtgcattg ggcaccggga tgcaccctcc ttctcatccc caccacgcca
ggctcctgag ccatccctct tcttccagga tccccctgga actagtatgg agagcctggc
cccctccact gactctctgc atggctcccc agtgctgatc ccccaagtgt gcatcgggca
ccgggatgca ccccgagcct cctccccacc ccgccaccca cccagtgacc tagcgttcct
ggcaccctca ccttcaccgg gcagctctgg gggctcccgg ggctcagcgc ctcccgggga
gaccaggcac aacttggagc gggaggagta cactgtgctg gccgacctgc ccccacccag
gaggctggcc cagagacagc cagggcccca ggcgcagtgc agcagcgggg gccgcaccca
cagccctggc cgtgcagagg tggagcgcct cttcgggcaa gagcgcagga agtccgaggc
agcgggggcc ttccaggccc aggacgaggg acggtcacag cagcccagcc aaggccagag
ccaacttctc cgaagacagt ccagccctgc ccccagcagg caggtgacca tgctccctgc
caaacaggca gaactgaccc ggcggagcca agcagagccc cctcatcctt ggagtcctga
gaagagacct gagggagatc ggcagctcca ggggtccccg ctgcccccca ggacatcagc
caggacccct gagagggagc tgcggacaca gagacctctg gagagtggcc aagcaggccc
aagacagcct ctgggggtgt ggcagagtca ggaggaaccg ccagggtccc agggccctca
tagacaccta gaaaggagct ggagcagcca ggagggaggc ctgggccctg ggggctggtg
gggatgtgga gagcccagcc tgggggcagc caaagccccg gagggagcat gggggggcac
ttccagggag tacaaggaga gctgggggca gccagaggcc tgggaggaga agcccactca
tgagctcccc agagaactag gaaagagaag cccactcacg agcccccctg agaactgggg
aggccccgca gagtcctcac aatcctggca ctctgggaca cccactgctg tgggctgggg
ggcagaggga gcgtgtccat acccgcgtgg ctctgagagg cgacccgagc ttgactggag
ggatctgctt ggccttctcc gggcaccagg agagggggtc tgggcccgtg tccccagcct
ggactgggag ggcctcttgg agctcctgca ggccaggctg ccccgcaagg acccagctgg
acacagggat gacctggcca gggctttagg gccagagctg ggtcccccag gcacaaacga
tgtccctgag caggagtcac acagccagcc agaaggctgg gccgaggcca ccccagtcaa
tggacacagc cccgcactgc agtcccagag cccggtccag ctgcccagcc ctgcctgcac
ctccacccag tggccaaaga tcaaagtgac aagaggacca gcgaccgcaa ctctggcagg
cctggagcag acgggccccc tggggagcag gagcactgcg aagggcccca gcttgccaga
gctgcagttc caaccagagg agcctgagga gtcagaacca agcagaggcc aagaccccct
gactgaccag aagcaggcag actcggcaga caagaggcca gcagagggca aggctgggag
cccgctcaag ggccgactgg tgacctcatg gcggatgccc ggggaccggc ccacgctgtt
caatccgttc ctgctgtctc tgggggtcct caggtggcga aggcccgatc tgctcaactt
caagaaggga tggatgtcga tcttggacga gcctggagag cctccctccc cctcgctcac
caccacctct acttcgcagt ggaagaaaca ttggtttgtg ctgacagatt caagtctcaa
atattacaga gactccactg ctgaggaggc agatgagctg gatggtgaga tcgacctgcg
ttcctgcacg gatgtcactg agtacgcggt gcagcgcaac tatggcttcc agatccacac
caaggatgct gtctatacct bitcggccat gacctcaggc atccggcgga actggatcga
ggctctgaga aagaccgtac gtccaacttc agccccagat gtcaccaagc tctcggactc
taacaaggag aacgcgctgc acagctacag cacccagaag ggccccctga aggcagggga
gcagcgggcg ggctctgagg tcatcagccg gggtggccct cggaaggcgg acgggcagcg
tcaggccttg gactacgtgg agctctcgcc gctgacccag gcttccccgc agcgggcccg
caccccagcc cgcactcctg accgcctggc caagcaggag gagctggagc gggacctggc
ccagcgctcc gaggagcggc gcaagtggtt tgaggccaca gacagcagga ccccagaggt
gcctgctggt gaggggccgc gccggggcct gggtgccccc ctgactgagg accagcaaaa
ccggcttagt gaggagatcg agaagaagtg gcaggagctg gagaagctgc ccctgcggga
gaataagcgg gtgcccctca ctgccctgct caaccaaagc cgcggagagc gccgagggcc
cccaagtgac ggccacgagg cactggagaa ggaggttcag gctcttcggg cccagctgga
ggcgtggcgt ctccaagggg aggctcctca gagtgcactg agatcccagg aggatggcca
catccccccg ggctacatct cacaggaggc atgtgagcgc agcctggcag agatggagtc
ctcgcaccag caggtgatgg aggagctgca gcggcaccac gagcgggagc tgcagcgcct
gcagcaggag aaggagtggc tcctggctga ggagacggca gccacggcct cagccattga
agccatgaag aaggcctacc aggaagagct gagccgagag ctgagcaaaa cacggagtct
ccagcagggc ccggatggcc tccggaagca gcaccagtca gatgtggagg cactgaagcg
agagctgcag gtgctatcgg agcagtactc gcagaagtgc ctggagattg gggcactcat
gcggcaggct gaggagcgcg agcacacgct gcgccgctgc cagcaggagg gccaggagct
gctgcgccac aaccaggagc tgcatggccg cctgtcagag gagatagacc agctgcgcgg
cttcattgcc tcgcagggca tgggcaatgg ctgcgggcgc agcaacgagc ggagttcctg
cgagctagag gtgctgcttc gcgtaaaaga aaacgaactc cagtacctaa agaaggaggt
gcagtgcctc cgggacgagc tccagatgat gcagaaggac aagcgcttca cctcgggaaa
gtaccaggac gtctatgtgg agctgagcca catcaagaca cggtctgagc gggagatcga
gcagctgaag gagcacctgc gtcttgccat ggccgccctc caggagaagg agtcgatgcg
caacagcctg gctgagtag
Human Full-length Wildtype TRIOBP Protein Isoform 1
(SEQ ID NO: 30)
MTPDLLNFKKGWMSILDEPGEPPSPSLTTTSTSQWKKHWFVLTDSSLKYYRDSTAEEADELDGEIDLRSCT
DVTEYAVQRNYGFQIHTKDAVYTLSAMTSGIRRNWIEALRKTVRPTSAPDVTKLSDSNKENALHSYSTQKG
PLKAGEQRAGSEVISRGGPRKADGQRQALDYVELSPLTQASPQRARTPARTPDRLAKQEELERDLAQRSEE
RRKWFEATDSRTPEVPAGEGPRRGLGAPLTEDQQNRLSEEIEKKWQELEKLPLRENKRVPLTALLNQSRGE
RRGPPSDGHEALEKEVQALRAQLEAWRLQGEAPQSALRSQEDGHIPPGYISQEACERSLAEMESSHQQVME
ELQRHHERELQRLQQEKEWLLAEETAATASAIEAMKKAYQEELSRELSKTRSLQQGPDGLRKQHQSDVEAL
KRELQVLSEQYSQKCLEIGALMRQAEEREHTLRRCQQEGQELLRHNQELHGRLSEEIDQLRGFIASQGMGN
GCGRSNERSSCELEVLLRVKENELQYLKKEVQCLRDELQMMQKDKRFTSGKYQDVYVELSHIKTRSEREIE
QLKEHLRLAMAALQEKESMRNSLAE
Human Full-length Wildtype TRIOBP Protein Isoform 4
(SEQ ID NO: 31)
MIPRRPREGPRADSSQRAPSLLTRSPVGGDAAGQKKEDTGGGGRSAGQHWARLRGESGLSLERHRSTLTQA
SSMTPHSGPRSTTSQASPAQRDTAQAASTREIPRASSPHRITQRDTSRASSTQQEISRASSTQQETSRASS
TQEDTPRASSTQEDTPRASSTQWNTPRASSPSRSTQLDNPRTSSTQQDNPQTSFPTCTPQRENPRTPCVQQ
DDPRASSPNRTTQRENSRTSCAQRDNPKASRTSSPNRATRDNPRTSCAQRDNPRASSPSRATRDNPTTSCA
QRDNPRASRTSSPNRATRDNPRTSCAQRDNPRASSPSRATRDNPTTSCAQRDNPRASRTSSPNRATRDNPR
TSCAQRDNPRASSPNRAARDNPTTSCAQRDNPRASRTSSPNRATRDNPRTSCAQRDNPRASSPNRATRDNP
TTSCAQRDNPRASRTSSPNRATRDNPRTSCAQRDNPRASSPNRTTQQDSPRTSCARRDDPRASSPNRTIQQ
ENPRTSCALRDNPRASSPSRTIQQENPRTSCAQRDDPRASSPNRTTQQENPRTSCARRDNPRASSRNRTIQ
RDNPRTSCAQRDNPRASSPNRTIQQENLRTSCTRQDNPRTSSPNRATRDNPRTSCAQRDNLRASSPIRATQ
QDNPRTCIQQNIPRSSSTQQDNPKTSCTKRDNLRPTCTQRDRTQSFSFQRDNPGTSSSQCCTQKENLRPSS
PHRSTQWNNPRNSSPHRTNKDIPWASFPLRPTQSDGPRTSSPSRSKQSEVPWASIALRPTQGDRPQTSSPS
RPAQHDPPQSSFGPTQYNLPSRATSSSHNPGHQSTSRTSSPVYPAAYGAPLTSPEPSQPPCAVCIGHRDAP
RASSPPRYLQHDPFPFFPEPRAPESEPPHHEPPYIPPAVCIGHRDAPRASSPPRHTQFDPFPFLPDTSDAE
HQCQSPQHEPLQLPAPVCIGYRDAPRASSPPRQAPEPSLLFQDLPRASTESLVPSMDSLHECPHIPTPVCI
GHRDAPSFSSPPRQAPEPSLFFQDPPGTSMESLAPSTDSLHGSPVLIPQVCIGHRDAPRASSPPRHPPSDL
AFLAPSPSPGSSGGSRGSAPPGETRHNLEREEYTVLADLPPPRRLAQRQPGPQAQCSSGGRTHSPGRAEVE
RLFGQERR
Human Full-length Wildtype TRIOBP Protein Isoform 6
(SEQ ID NO: 32)
MEEVPGDALCEHFEANILTQNRCQNCFHPEEAHGARYQELRSPSGAEVPYCDLPRCPPAPEDPLSASTSGC
QSVVDPGLRPGPKRGPSPSAGLPEEGPTAAPRSRSRELEAVPYLEGLTTSLCGSCNEDPGSDPTSSPDSAT
PDDTSNSSSVDWDTVERQEEEAPSWDELAVMIPRRPREGPRADSSQRAPSLLTRSPVGGDAAGQKKEDTGG
GGRSAGQHWARLRGESGLSLERHRSTLTQASSMTPHSGPRSTTSQASPAQRDTAQAASTREIPRASSPHRI
TQRDTSRASSTQQEISRASSTQQETSRASSTQEDTPRASSTQEDTPRASSTQWNTPRASSPSRSTQLDNPR
TSSTQQDNPQTSFPTCTPQRENPRTPCVQQDDPRASSPNRTTQRENSRTSCAQRDNPKASRTSSPNRATRD
NPRTSCAQRDNPRASSPSRATRDNPTTSCAQRDNPRASRTSSPNRATRDNPRTSCAQRDNPRASSPSRATA
DNPTTSCAQRDNPRASRTSSPNRATRDNPRTSCAQRDNPRASSPNRAARDNPTTSCAQRDNPRASRTSSPN
RATRDNPRTSCAQRDNPRASSPNRATRDNPTTSCAQRDNPRASRTSSPNRATRDNPRTSCAQRDNPRASSP
NRTTQQDSPRTSCARRDDPRASSPNRTIQQENPRTSCALRDNPRASSPSRTIQQENPRTSCAQRDDPRASS
PNRTTQQENPRTSCARRDNPRASSRNRTIQRDNPRTSCAQRDNPRASSPNRTIQQENLRTSCTRQDNPRTS
SPNRATRDNPRTSCAQRDNLRASSPIRATQQDNPRTCIQQNIPRSSSTQQDNPKTSCTKRDNLRPTCTQRD
RTQSFSFQRDNPGTSSSQCCTQKENLRPSSPHRSTQWNNPRNSSPHRTNKDIPWASFPLRPTQSDGPRTSS
PSRSKQSEVPWASIALRPTQGDRPQTSSPSRPAQHDPPQSSFGPTQYNLPSRATSSSHNPGHQSTSRTSSP
VYPAAYGAPLTSPEPSQPPCAVCIGHRDAPRASSPPRYLQHDPFPFFPEPRAPESEPPHHEPPYIPPAVCI
GHRDAPRASSPPRHTQFDPFPFLPDTSDAEHQCQSPQHEPLQLPAPVCIGYRDAPRASSPPRQAPEPSLLF
QDLPRASTESLVPSMDSLHECPHIPTPVCIGHRDAPSFSSPPRQAPEPSLFFQDPPGTSMESLAPSTDSLH
GSPVLIPQVCIGHRDAPRASSPPRHPPSDLAFLAPSPSPGSSGGSRGSAPPGETRHNLEREEYTVLADLPP
PRRLAQRQPGPQAQCSSGGRTHSPGRAEVERLFGQERRKSEAAGAFQAQDEGRSQQPSQGQSQLLRRQSSP
APSRQVTMLPAKQAELTRRSQAEPPHPWSPEKRPEGDRQLQGSPLPPRTSARTPERELRTQRPLESGQAGP
RQPLGVWQSQEEPPGSQGPHRHLERSWSSQEGGLGPGGWWGCGEPSLGAAKAPEGAWGGTSREYKESWGQP
EAWEEKPTHELPRELGKRSPLTSPPENWGGPAESSQSWHSGTPTAVGWGAEGACPYPRGSERRPELDWRDL
LGLLRAPGEGVWARVPSLDWEGLLELLQARLPRKDPAGHRDDLARALGPELGPPGTNDVPEQESHSQPEGW
AEATPVNGHSPALQSQSPVQLPSPACTSTQWPKIKVTRGPATATLAGLEQTGPLGSRSTAKGPSLPELQFQ
PEEPEESEPSRGQDPLTDQKQADSADKRPAEGKAGSPLKGRLVTSWRMPGDRPTLFNPFLLSLGVLRWRRP
DLLNFKKGWMSILDEPGEPPSPSLTTTSTSQWKKHWFVLTDSSLKYYRDSTAEEADELDGEIDLRSCTDVT
EYAVQRNYGFQIHTKDAVYTLSAMTSGIRRNWIEALRKTVRPTSAPDVTKLSDSNKENALHSYSTQKGPLK
AGEQRAGSEVISRGGPRKADGQRQALDYVELSPLTQASPQRARTPARTPDRLAKQEELERDLAQRSEERRK
WFEATDSRTPEVPAGEGPRRGLGAPLTEDQQNRLSEEIEKKWQELEKLPLRENKRVPLTALLNQSRGERRG
PPSDGHEALEKEVQALRAQLEAWRLQGEAPQSALRSQEDGHIPPGYISQEACERSLAEMESSHQQVMEELQ
RHHERELQRLQQEKEWLLAEETAATASAIEAMKKAYQEELSRELSKTRSLQQGPDGLRKQHQSDVEALKRE
LQVLSEQYSQKCLEIGALMRQAEEREHTLRRCQQEGQELLRHNQELHGRLSEEIDQLRGFIASQGMGNGCG
RSNERSSCELEVLLRVKENELQYLKKEVQCLRDELQMMQKDKRFTSGKYQDVYVELSHIKTRSEREIEQLK
EHLRLAMAALQEKESMRNSLAE

A non-limiting example of a human wildtype TRIOBP genomic DNA sequence is SEQ ID NO: 33. The exons in SEQ ID NO: 33 are: nucleotide positions 1-102 (exon 1), nucleotide positions 601-709 (exon 2), nucleotide positions 4319-4492 (exon 3), nucleotide positions 13440-13579 (exon 4), nucleotide positions 16223-16424 (exon 5), nucleotide positions 18776-18947 (exon 6), nucleotide positions 26198-29516 (exon 7), nucleotide positions 36311-36425 (exon 8), nucleotide positions 37412-38455 (exon 9), nucleotide positions 41655-41732 (exon 10), nucleotide positions 43908-44045 (exon 11), nucleotide positions 54785-54841 (exon 12), nucleotide positions 57890-57997 (exon 13), nucleotide positions 58114-58203 (exon 14), nucleotide positions 58563-58672 (exon 15), nucleotide positions 60626-61151 (exon 16), nucleotide positions 62167-62277 (exon 17), nucleotide positions 68683-68830 (exon 18), nucleotide positions 71087-71189 (exon 19), nucleotide positions 72041-72200 (exon 20), nucleotide positions 72275-72388 (exon 21), nucleotide positions 74663-74749 (exon 22), nucleotide positions 75614-75777 (exon 23), and nucleotide positions 76796-79569 (exon 24). The introns are located between each pair of these exons in SEQ ID NO: 33, i.e., at nucleotide positions 103-600 (intron 1), nucleotide positions 710-4318 (intron 2), nucleotide positions 4493-13439 (intron 3), nucleotide positions 13580-16222 (intron 4), nucleotide positions 16425-18775 (intron 5), nucleotide positions 18948-26197 (intron 6), nucleotide positions 29517-36310 (intron 7), nucleotide positions 36426-37411 (intron 8), nucleotide positions 38456-41654 (intron 9), nucleotide positions 41733-43907 (intron 10), nucleotide positions 44046-54784 (intron 11), nucleotide positions 54842-57889 (intron 12), nucleotide positions 57998-58113 (intron 13), nucleotide positions 58204-58562 (intron 14), nucleotide positions 58673-60625 (intron 15), nucleotide positions 61152-62166 (intron 16), nucleotide positions 62278-6862 (intron 17), nucleotide positions 68831-71086 (intron 18), nucleotide positions 71190-72040 (intron 19), nucleotide positions 72201-72274 (intron 20), nucleotide positions 72389-74662 (intron 21), nucleotide positions 74750-75613 (intron 22), and nucleotide positions 75778-76795 (intron 23).

| Human Wildtype TRIOBP Genomic Sequence (NCBI Accession No. NG_012857.1) | |
|---|---|
| (SEQ ID NO: 33) | |
| 1 | cttccacctc ccgagcatcg ctctgattca ttgttaaaat aaagaggcca tgaccctggc |
| 61 | actcccaccc cggcagcccc tgtgcgtgga aacccagcca aggttagcac tgggcctcag |
| 121 | aaaagaggag ctggtagggg gaggcctaca ggaggggtag gggtcgaggg atccggggag |
| 181 | gaactggctg aatgcctgga tggagagggt cccgggtcca ttctctgggt ggggcatgtg |
| 241 | tagctgctta aggttttgat tattatatcc acatctcaag tgcattttag agaaggattc |
| 301 | tgcctattgg ttgtgggtag gggaggaaac gggagcagcg aggggcacgt ggagtagagc |
| 361 | cccctaccca cccccaggaa aatgagcggc gttctcctgc taggcgacgc tcctttatgt |
| 421 | ggccagacca ggagagaaac gcccaggacg ggcggggccg atgcgcccct cttccaccac |
| 481 | caggagtcgg gaggacgacc gcccggggag gatcagagac cctggggcca cggaaggacc |
| 541 | agtgggtagc cctctcccca ccgtgccttg ctgtcgcccc caccgcattc ccttctccag |
| 601 | gtctgatgat ggaggagcct tggccctggc tgtcccaggg gaggaggtga aattcctcag |
| 661 | ctctccacca agattggcca caaaagcctg atcccctgga acacagcagg tgaggatgga |
| 721 | tgtgggcagg tgacctgggg gcaccaaaag ctgcaggtgt tttgggatgt tgggttcctc |
| 781 | ccacccccca ccctccccct ggggctctgt gtgagtgggg aggccatctt taagtgattc |
| 841 | tcctgaccca gctccactcc agggcatgga tgttggtggc cttaggggca cccagaaagg |
| 901 | agaacccttg cacaagtaag ataccagggc ggccgggcgc ggtggctcaa gcctgtaatc |
| 961 | ccagcacttt gggaggtcga ggtgggtgga tcacaaggtc aggagttcga gaccagcctg |
| 1021 | gcccatatgg tgaaacctcg tctctaccaa aaatacaaaa aaattatccg ggcatggtgg |
| 1081 | cacatgcctg taatcccagc tatttgggag gctgcggcag gagaattgct tgaatccggg |
| 1141 | aggcagaggt tgcagtgagc cgagatcgcg ccactgcact ccagcctggg tgatagaccg |
| 1201 | agattctgtc tcaaaaaaaa aaaaaaaaaa agaaagaaag acaccagggt ttcagaaggg |
| 1261 | tatgatgctt cacacctgta atcccagcac tttgagggaa ggaggaggat tacttgaggt |
| 1321 | caggagttca ggagttcaaa accagcctgg gtaacactgc aagagccttt tttttttttt |
| 1381 | tttttttttt tgagacacac tgttgcccag gctggagtgc agcggggcga tcttagctcc |
| 1441 | actgcaacct ccacctcctg ggttcaagca attctcttgc cccagcctct tgagtagctg |
| 1501 | ggattacagg catgcgcacc acgcccagct aatttttata gttttagtag agacagggtt |
| 1561 | tcatcatgtt gggcaggctg gtcttgaatt cctggcctca agtgatctgc ctgcctcagc |
| 1621 | ctcccaaagt gctgggatta taggcgtgag ccaccgcacc tggccaagag cccattttta |
| 1681 | caaaataaaa aagaaaaaaa ttagctgggc gtggtggcgt acacctgtag tcccagttac |
| 1741 | tcagtcccag agactgaggc aggagaatca tttgagcccg ggagtttgag gttgcaagga |
| 1801 | gctatgatcg agccactgca ctccagcctg ggtgacagag caagaccctg cccccacctc |
| 1861 | cccaaaaaag acattagggg ccggatgcag tggctcacac ctgtaatccc agcattttgg |
| 1921 | gaggccgaag ggggcggatc acctgaggtc aggagttcac ctggccaaca tggcgaaacc |
| 1981 | ccgttgctac taaaaataca aaaattagcc aggcatgacg gtgcatgcct gtgatcccag |
| 2041 | atactactgg ggaggctgag gtagaattgc ttgaacccag gaggcggagg ttgcagtgag |
| 2101 | ccgagtgagc caagatcaca tcacggcact ccagcctagg tgacagagag agacttcgtc |
| 2161 | tcaaaaaaac aaaacaaaac aaaacgaaaa cacattagga tttctttttg gctaagttct |
| 2221 | gccacaaact tgctgtgtga ctttaggctg ttcacgtttg tcaaagacgt cgtcctcaac |
| 2281 | tctgaaaata cctggccagc tttttgcctc cctcgctgtg cttcccaagg gacctgtgcg |
| 2341 | agttggcagt atgatccagt gctcctcact tccacctccg agagagcagg gacacacaca |
| 2401 | gccctggatt tgaatcagcc actgcgacct tcagtgaccg aaggaacctc tctgagcctt |
| 2461 | agttgcctga tctaaaattg gcattaattt gatctacaat gcagctataa gacaaggaca |
| 2521 | ctcaaacttt ggggcgcact agaatcacct ggggaatcac tttttttttt taagacagag |
| 2581 | tctcactctt atcacccagg ctggagtaca gtggtgcaat cttggctcac tgcaacctcc |
| 2641 | gcctcctggg ttcaagtgat tctcctgcct cagcctcccg agtaactggg actacaggtg |
| 2701 | cataccacca tgcctggcta attttgtatt tttagtagag acagggtttg gcaatattgg |
| 2761 | ccaggctggt ctcgaactcc tgacctcagg tgatctgccc gcctcggcct cccaaagtgc |
| 2821 | tgggattata ggcgtgagcc actgcgcctg gccaggaatc gcttctttat tttcattttc |
| 2881 | atttatttat ttatttattt atttattttg agatggagtc tcgctctgtt gctcagtctg |
| 2941 | gagtgaagtg gcgtgctctc cgctcactgc tacctctgcc tcccaggttc aagcgattct |
| 3001 | cgtgcctcag cctcccaagt agctgggact acaggtgcgt gctaccacgc ccagctaatt |
| 3061 | tttgtattta gtagaggcgg ggtttcacca tgttggccag gctggtcttg aactcctggc |
| 3121 | ctcaagtgat ctgcctgcct cagcctccca aagtgctggg attacaggcg tgagccaccg |
| 3181 | ccctcggctg gaatcacttc tttaacatgc acagccccag acctcagtct cttggagatt |
| 3241 | ctaactgtgt gaattgggag agagaaaggg gaatctagat tttttttttt tttttttgag |
| 3301 | atgtggtctt gctatgttgc ctaggctgga gtgctgtaac tattcacagg tgcagttata |
| 3361 | gcacactacc acctcaaact cctgggctca agagatcctc ccacctctgc ctcccgagta |
| 3421 | gcttggactg caggtgcaca ccatcttacc cagctgaaat ctgctttttt tttttctcag |
| 3481 | tgcagtggca cagtcatggc tcactgcagc ctcgacctcc cagactcagg tgatcctccc |
| 3541 | aacttcagcc tcctgagtag cagggaccac aggcctgcac caccttgccc agctaatttt |
| 3601 | tgtatttttt gtagagacgg agtttcgcca gttgcccagg atggtcttga actcttgggc |
| 3661 | tcaaatgatc cgccgcccgc aatgggccca gctggaatct gcattttttt gttttttgtt |
| 3721 | ttgagatgaa gtctcactct gtcgcccagg ctggagtgca atggcgcaat cttggctcac |
| 3781 | tgcaacctcc gcctcctggg ttcaagccat tctcctgcct caggctccca agtagctggg |
| 3841 | attacaggca cgtgccatca cacttggctg atttttgtat ttttagtaga gacagggttt |
| 3901 | taccatattg gccaggctgg tctcgaactc ctgacctcaa gtgatcggcc cacctcggcc |
| 3961 | tcccaaagtg ctgggattac aggcatcagc cactgtgcct ggcctggtat ctgcattttt |
| 4021 | atgaacagcc caggagggtg taatgcagag tccttggcgt gcactttcag caacactgca |
| 4081 | gtaaggacta gaaatcgcac atcaaatgct ggcagagtgc ctggtgctta gtaggtgctt |
| 4141 | agtgagtgtt tgttgagtga ataaatgaac aggaatggat ggagaacagg aacccaaagg |
| 4201 | aatggatgag taaccttatt tccttggtcc ttgtctggaa actggggcaa ctggggctgg |
| 4261 | ggttcaggtg agtggccagc cagtcatctg gtctttgcct ccccctcatt tttgccaggc |
| 4321 | ctcacataga cggtcagcca ttggatcata ggaactgccc tggcctgact cacccaatat |
| 4381 | ggaggaggtg cctggggatg ccctgtgtga acactttgag gccaacatac ttacccagaa |
| 4441 | ccgctgtcaa aactgcttcc accctgagga ggcccatgga gcaagatacc aggtgggcca |
| 4501 | gttttccacg tggttggggt ggtttgtgat gggggcatgg gtgaaggact gggcctgtgg |
| 4561 | tgtccgctaa ctcgcagttg aacccagatg ctggacttcc tcctgtcgag agcctcggtt |
| 4621 | ttcccacatt gcagggaagt ggttgaacca tgtgaatgtt tcacaatctt cagtcacttt |
| 4681 | cctcctgctt atatgatacg attgttacca tagccttatc taatctgtta cttactttgt |
| 4741 | tattctttaa atcaacatct ttttcccctt ctattgaaac aaaacttcac atcatcaaca |
| 4801 | caagtagaaa actagaatta cttcctgtta agaagaagag gttaagaaga aaggtagagg |
| 4861 | ctgggcacgg tggctcacgc ctgtaatccc agcactttgg gaggccgagg tgggtggatc |
| 4921 | acccgaggtc gggagtttga aaccagcctg gccaacatgg tgaaaattag ctgggcatgg |
| 4981 | tggtgcatgc ctgtaatccc agctacttgg gaggctgagg caggagaatc acttgaacct |
| 5041 | gggaggcgga gattgtggtg agccgagatc gcgccattgc actccagcct gggcaacaga |
| 5101 | atgaaactgt ctcaaaaaac aaaacaaaac aaaacaaaaa agagaagaaa ggtagaaaaa |
| 5161 | gaaaaaacaa aacagaaatg tcgttacacc ctaacttgag agtgttttct ttttcttttt |
| 5221 | cttttttttg agacagagtc tcactctgtc gcctgggcta gagtgcaatg gcaatggtgt |
| 5281 | gatctcagct cactgcaatc tctgcctcct aggttcaagc gatcctcccg cctcagcctc |
| 5341 | ccaagtattg ggattacagg tgcctgccac catgcccagc taatttttgt atttttagta |
| 5401 | gaaacggggt ttcgccatat tggccaggct ggtcttaaac tcctgacctc aggtgatcca |
| 5461 | cccgcctcag cctcccaaag tgttgggatt acaggcgtga gccaccgcgc ctggcctcga |
| 5521 | gagtgttttc tatggaaggc tctgagcttg catcctgctg tctctttgtc aaaaagggag |
| 5581 | attagcagat gttagagaga tgttaagaat acaccagcat catctagaga ttttctttct |
| 5641 | ctctctcttt tttttttttt tttttttttt tggagatagg gtcttgctct gtagcacagg |
| 5701 | ctggagtgca gtggtccaac ctatagtaca tggctcactg tagcctcgac ctcccaggct |
| 5761 | caaatgatct tcccaccttg gcctcctgag tagcagggac tacaggcaca tgccaccatg |
| 5821 | gccagctaat tttcttgtaa agatgggatc tcactatgtt gccagggctg gtctcaaatt |
| 5881 | cctgggctca ggtgatcctc ccacctcaac ctctgtaact gcagggatta caggtgtgag |
| 5941 | acactgcacc tggtctcaag attttcttct tcaagttatt gaaggagtga agaagaatcc |
| 6001 | ttttttttct ttcttttttt ttcatttgag acggagtctc tctctgtcgc caggctagag |
| 6061 | ttcagtggct caatctcagc tcactgcaac ctccgtctcc tgggttcaag caattctcct |
| 6121 | gcctcagcct cctgagtacc tgggattaca ggtgcccacc accatgccca gctaattttt |
| 6181 | gtatctttag tagagacagg gtttcaccat gttggtcagt ctggtctcga actcctgacc |
| 6241 | ttgtgatctg tctgcctcgg cttcccaaag tgctgggatt acaggtgtga gccaccgcgc |
| 6301 | ctggccgaga ttttcttctt caagttattg aaggagtgaa gaagagtcct tttctgactt |
| 6361 | tgtgattgat gtagtctgct gccaagcccc catgcatgta aacacagagc acctgataca |
| 6421 | cacgcaatga gaaacctggg actcacgaac ctccgagggc ccttccaacc ttgaccacca |
| 6481 | tggcttcgtc attcccattg gggtctgaag gtcctctttt tttttttttt tttttgagac |
| 6541 | agagtctcgt tctgttgccc aggctggagt acagtggtgc gatctgggct cactgcaagc |
| 6601 | tcggcctccc aggtttatgc cattctcctg cctcagcctc atgagtagct gggacaacag |
| 6661 | gcacccggca ccacgcccag ctaatttttt gtctttttag tagagacagg gtttcaccat |
| 6721 | gttagccagg atggcctcga tcttcctgac ctcgttattc gcccacctcg acctcccaaa |
| 6781 | gtgctgggat tacaggcatg agccaccgcg cccagcttct accagtcttg atacctccaa |
| 6841 | taatagctct cttgtgctca cctgcaagtt caggttcagg gaacagatgc ctgggagtcc |
| 6901 | atctttcatg caaatgactt tcttccagtg gttgggaaga tggcaagaat ggaacaggag |
| 6961 | agccgtggat tcaggctcgt ctgagttcta gtctcaatcc caccatttcc cacccgagac |
| 7021 | cgtgggtcag ctcacaaatt catacgtact gaataggtgc ttaaaatgtg ccaggcatta |
| 7081 | tgctagatga tggggaaatc atgataaatc agaaaggcac agccaggcac agtgactcac |
| 7141 | tcctgtaatc ccagcacttt gggaggccaa ggtgggtgga tcacttggag tttgagacca |
| 7201 | tcctggcaac atggcaaaac cccatctcta caaaaaattt aaaaaattag ctgggcgtag |
| 7261 | tgggatgcgg ctatagtctc agttactcag gaggctgagg tgggagaatc acctgagact |
| 7321 | gggaggcggg ggcttcagtg agccgtgatc gcattactgt atttcagcct gggtgacaga |
| 7381 | gttggaccct gactcagaac agaacagaac agaacagaac agaacagaac agaacagaac |
| 7441 | agaacagaac agtacagaaa acagggagaa ggaggggaag ggggagggga agggggaggg |
| 7501 | ggagagagag gaggagagga ggagagaggg acacatccct ggcttgtctt catggggctt |
| 7561 | atattctagc aagagaaatg cacatgatta tttaatcttg gttgtgataa atgtataaag |
| 7621 | gaacagtaag tggcatgaag gtaaaggata gggcattagg gaaagtttct ccaaaaaaaa |
| 7681 | aaaaatgaca tttcagctgc ttagtaaagg ctgtgcaggc attagcatta gtgaaattgc |
| 7741 | aggaagagga gaggaaggaa ggagaatgtg caaaggccag cctggcacgg cagctcacgc |
| 7801 | ctgtaatccc agcactttgg gaggaagtgc ttggattgag gcaccgaagc aggaagatca |
| 7861 | cttgaactca ggagtttgag accagcctgg gcagcacagt gagaccccat ttctaccaaa |
| 7921 | aaaaaaaaaa aaaaaattag ccaggcagga tggtgcacac ctgtagtccc agctagtcag |
| 7981 | aaggctgatg tgggaggatt actggatccc aggagttcaa ggttgcagtg agccatgagc |
| 8041 | atgccaatgc actccagcct gggcaacaga atgagacctt gtctcaaaaa caaaacaaac |
| 8101 | aaacaggtca ggtgcagtgg ttcatgcctg taatctcagc actttacgag gccgagacgg |
| 8161 | gtggatcacc tgaggtcagg agttcaagac caccctggcc aacatggtga aaccccatct |
| 8221 | ctattaaaaa tacacaaatt agctacatgt ggtggtgggt gcctgtaatc ccaggtactt |
| 8281 | aggaggctga ggcaggagaa tctcttgaac ccaggaggca gaggttgcag tgagccaaga |
| 8341 | ttgcaccact gtactccagc ctgggcaaca gagcgagaat cctctcaaaa caaaacaaac |
| 8401 | aaacaaagaa tatgcaaagg ccctgaggca gaaaggaggc tgcacatcgg gctggagagg |
| 8461 | acaggcaaga ggggggcaca gaggagctat aggacaagga tggagaatgg ggggaggcct |
| 8521 | ggtctcactg gccttgtgcg gctttgtgga catgttgagg acttaggatt ctgaggtttt |
| 8581 | tttctgtttt gtttttgttt ttttttgaga tggagtctcg ctctgtcgca caggctggag |
| 8641 | tgcagaggcg tgatctcggc tcactgcaac ctctgcctcc caggttcaag cgattctcct |
| 8701 | gcctcagcct cccaggtagc tgagactaca ggcatgtact accaaaccca gttaattttt |
| 8761 | gtatttttag tagagatggg gtttcaccat gccgctcagg ctggtcttga actcctggcc |
| 8821 | tcaagcaatc tgccctcctt ggcctcccaa agtgctgaga ttacaagtgt gagccacctc |
| 8881 | gcccggccaa gccacggaag atttcaaata agggagtgac gtgctctgat ttgcattttt |
| 8941 | cacagtcact ctgattgctg ggcagagaga atggagggca gggtggtgag agtgaatcag |
| 9001 | gtgacccgcc aggaaaaggt gcacgtactc atcggtgcca gagatgattg gggctttgag |
| 9061 | atggaggcaa atggacacat ttccgaacta attgagcagg cacgtctaca ggatttgggg |
| 9121 | gtgagggagg ggagaaaccc atggctgact cctgtaattt cttcaggcag ggtacttggg |
| 9181 | ttgggaggtc tgtttggagt tgtctggggc atggtgtggg gtgtcggcag gatacagtgg |
| 9241 | agatgagggg gaagttagtg aattggtggg gctggagctc agagccagac catcacagga |
| 9301 | gatggaggcc tgcgagtctt caggatctgt cactgaccaa agtcttgctc acggatgagc |
| 9361 | ctatcatctg tggaagaaag tgtggaatga gatgagaaca ggcctgggtg gaggccaggg |
| 9421 | aatcagcaga gacagccaag aaggccagag gctaggaggc aaaccccaca gtgtgcccct |
| 9481 | cctccccagg agccaagacc agagatggtt tcaagagagg gggccaggca caatggctcc |
| 9541 | cgcctgtgat cacagcactt taggaggctg aggctaaagg atggcttgaa accaggagtt |
| 9601 | taagaccagc ctagttaaca aagtgagatc ccatctctac aaaacaaaaa atgaactggg |
| 9661 | tgtagtggtg catgcctgca gtcccagcta cttgggaggc tgaggcagga ggattgtttg |
| 9721 | agcccaggag attgaggctg cagtgcactg tgatcatgcc actgccacag caagactatc |
| 9781 | ttaaaaaaaa aaaaaaaaga tcagagaggg gcacgacatc aaatcctcca ctttcttgag |
| 9841 | cttccagttg ctctgatgat gatcaaggct gtgaaactct cctttggcaa atgcatgagg |
| 9901 | ggtgactgtt gtgtctccga gccaggccct gggacccaac tctgggcagg tagcagctcc |
| 9961 | ctgtgtatac cagatgtgat agtaaaaaac tgacactggg ccgggcgcgg tggctcacgc |
| 10021 | ctgtaatccc agcactttgg gaggccgagg cgggtggatc acttgaggtc aggagtttga |
| 10081 | gaccagcctg actagcatgg tgaaatcctg actctactaa aaatacaaaa atacaaaaat |
| 10141 | taacagggtg tggtggcgga tgcctgtaat cccagctact cgggaggctg aggcaggaga |
| 10201 | atttcttgaa tccgggaggc ggaggttgca gtgagccgag atctggccac cgcactccag |
| 10261 | cctgggcgac aagagcgaaa aactccatct caaaaaaaac aaaaaaacct cctgggcccc |
| 10321 | agaagatggc aactgcctct tccagcctct tccacctgcc tgcctgtgca ctcctctgcc |
| 10381 | actctggacc tattccatgg tgccacgtgg tcaggcattg aaggaatggg tgggtttttt |
| 10441 | ttccatcacc ctcagatgct gctgctgggg ttggggcggg tcttccctgg ctgactctgt |
| 10501 | ctgcctcacc cactctctcc ccatctctct gtcaccacag gtctccaggg accaaattta |
| 10561 | gcccattttt cctccaagga ttctggaccc ttggccatat ttgcctctag ctttaggtca |
| 10621 | acagatttct aacatggagt caaaaaatct gtatgtttaa aaaaaaaaaa ttggctggga |
| 10681 | gtggtggctc acgtctgtaa tcccagcact ttgggaggcc gaggcaggtg gatcacctga |
| 10741 | ggtcaggagt tcgagaccac cctggccaac atggtgaaac cccgtctcta ctaaaaatac |
| 10801 | aaaaattagc tgggtatggt ggcggacccc tgtagtccca gctactcagg aggctgaggc |
| 10861 | aggacaattg cttgaacccg ggaggcagag gttgcagtga gctgagatcg cgccattgca |
| 10921 | ctccagcctg ggtgacagag tgagactctg tctcaaaaaa aagaaaaaaa aaaaaaaggc |
| 10981 | cgggcgtggt ggctcacggc tgtaatccca gcactttggg aggccgaggc gggcggatca |
| 11041 | cgagatcagg agatcgagac catcctggct aacacggtga aaccctgtct ctactaaaaa |
| 11101 | tacaaaaaat tagctgggca tggtggcggg cgtccatagt cccagctact cgggaggctg |
| 11161 | aggcaggaga atggcatgaa cccgggaggc ggagcttgca gtgagccaag atcacgccac |
| 11221 | tgcactctag cctgagcgac agagtgagac tccgtctcac aaaaaaaaaa aaaaaaaaaa |
| 11281 | attgaggcca ggcacagtgg ctcatgcctg tgatcccagc actttggccg aggcaggcag |
| 11341 | atcacctgcc atcaggagtt cgagaccagt ctcgccaaca tggcaaaccc ctgtctctac |
| 11401 | taaaaataga aaaattagcc gggcatggtg tcgggcacct gtaatcacag ctactggggt |
| 11461 | ggctgaggca gaagaattgc ttgaacccag gaggcgaagg ttgcagtgag ccgagactgc |
| 11521 | accattgcac tctaacctgg gctataagag cgagactctg tctcaaaaaa aaagcccttt |
| 11581 | acaaatatta actcgtttaa ttctcagaac acccctagga ggtgggtcct gctatcctgc |
| 11641 | cggttttgta gatgaggtaa ctgaggcaca gagaggataa gtcacgtggc ccaggtcttg |
| 11701 | gtcgcttctc agcctccatt tccgagagga gaaaacaatg ctccgaaagg ggaagtcatc |
| 11761 | ggcctgaggt caccagccgg gaaccggagg agccaggatg gggcccgagg tccatctgag |
| 11821 | gccagagcca gtgctgaccc gccagacagg aaatcagagc tggggactga ccgtggggcc |
| 11881 | cactcaagtg ggtggtgtgg ggtggtgacg ggggctgggg tgaaagccag gatgaggtgt |
| 11941 | cacattaggc tagggttcct gctgggcctg gcggggcaga gacccctggt tgatgcctgg |
| 12001 | caagtctctc ctcatttctg ctgtgctgtc ccctgggtgg ccaaggcgcc aggcccggct |
| 12061 | gtggatctga cacctcctgc tgccgagctc tgatcagaat ctggctgcag ggcagggggt |
| 12121 | gaaactgaat cccaccaggc ctcccctggc ctggatgtca cggcagacat cccaacccca |
| 12181 | cttcttggca ctgcctgacc ccagcagtgg ccttcttgga acctggagag cctgtgccca |
| 12241 | ccctgtggtg ccgaccaggc tgctgccttg gccctgggct ctgggctcca gggtccccag |
| 12301 | ggatcccagg cccaggctct tgcccctccc ggctctcagg tgagctgctg ggatcgggtt |
| 12361 | cctgcggttt catgtctttc attgttcttg gctggggccc ggcttatgaa actgtaatga |
| 12421 | ggggcccctg ctcccgaagc cggcccctct aaggtttcca ctgcctcggc ggctgctgcc |
| 12481 | accacagccc ggagtcgggg cctgggaggg cagcagtgtg ggggcctgag ccggagcccc |
| 12541 | ccggggacga gggtgctgac agtcgacagc caccaccacc accagagccc gcagcccagg |
| 12601 | taatccgaga ggaggcgggg cgcagggtgc catggccgct gtctcccacc accaggggag |
| 12661 | ggaactgccg ctctccagag gtggggagga attggggggg gccctggagg tgagcagccg |
| 12721 | ttcccccctg gtgagcccag caccagacca gccttcacag gtgtggcctt gccaaaggtg |
| 12781 | gagggagaga ggagtgggca gctggctcta gcccccgccc ttttcctggg gccttgaccc |
| 12841 | tccagccctg gatcccctgc ctctccttta gtggggcaaa tctcttctgt cctccaagac |
| 12901 | cttgctttcc catctcctct accatcaaac tgcctcttcc tccctcagac ctctcccttc |
| 12961 | tctcagccag cctaatcctt cctcctcgcc cttgtctttt ggccgcaaaa gaccctggtg |
| 13021 | gatccttcta tccacctcgt tctggggcgc acgcacatac atgcatgtat acatgaagtt |
| 13081 | gcactcatgc aaacgtacag atcggtgtac ccgcgggacc tgcgtagaca cgcatgcgca |
| 13141 | agagctcgct ccacctatgt gcacacatgt gcccccaata ccaggccttt atccacacac |
| 13201 | atacctgctc caaggcccat atgtgtacac tcacgtgtgc aggcacacac caggtacctc |
| 13261 | agccagccct cggcttgagt cccaaggggt gcttctagcc tgatgggcag ctccttgaga |
| 13321 | agtgcaggat cccccgagga gatgcctgga gactcccacg ccaccgggag gggctgtgca |
| 13381 | gggggagggg agcccccacg tgggcgctga gctgtctcct ctctccaacc ctggcccagg |
| 13441 | agctcaggag cccttcaggt gctgaggtgc cctactgcga cctgcctcga tgtccacctg |
| 13501 | cccctgagga cccactcagc gcctcaacct ccggctgcca gtctgtggtg gacccaggcc |
| 13561 | tcaggccagg gcccaagagg tgggtagagt cccagggccc aggaagggct tcatggggtg |
| 13621 | gaatgccctc acccttccca tttgcactcc ttccccaagg cagcacctgt ctctaatggc |
| 13681 | tgctggcatg ggtcacgtgg tcagtcctct aaacctaggc acaggtgggt ggtgggcatg |
| 13741 | ctccttgggc ccacaggaag aactcagatc acaccaccct gtttgcactt cagtggctga |
| 13801 | gcctcccctc cccagtcctg gctgtctctc ccaggggccc tgcccacccc ataacctccc |
| 13861 | aagaccgagc tcagctttgc cccagggtgt gtggctgctg tacactcacc ttgcagtggg |
| 13921 | catggccaca gcctactcac atttcctggt tccctttgca cagctttaaa agattaaaca |
| 13981 | gtccctgcac ccaggggcac ctgctgtcac ccacctacgt gggtgtgcac tgctccaggc |
| 14041 | tctggggttc cttccaccct caggcctgag tggcagtccc cctttaaggt catggtacct |
| 14101 | tgggaggcca tggtctgaga gtctagaaac ctgggttcca gcttcagatc catccctgat |
| 14161 | gggtcttgag caagttactc tgtctttctg tgtctcagtt tccctctctg taaaataaga |
| 14221 | gggattgaat taaatcagca ttgtgctggg atcattcatg tgctcctcac aggtaggcat |
| 14281 | attaaaacaa acaaacaaac aaaaaaacag cctgggtgca gtggctcatg cctgtaattc |
| 14341 | cagaactttg ggaggccaag gtgggcggat cacttgaggt caggaggtcg agaccagcct |
| 14401 | gaccaacatg gcgaaaccct gtctctacta actatacaaa aattagccag gcatggtggc |
| 14461 | aggtgcctgt aatcccagct gctcgggagg ctgaggcagg agaaccactt gaacccggga |
| 14521 | ggcagaggtt gcagtgagtt gagatcgtgc cattacactc cagcctgggt gacacagcga |
| 14581 | ggctccgtct caaaaaaaaa aaaaacaaca aaaaaaaaaa acaaaaaaaa acccacaaaa |
| 14641 | aaacgaaaaa aaaaacaaag acagctttgt tatcaagtat gtttcagaaa gtctgggata |
| 14701 | aatgaagttg aatagctttc ctgcaggact tgtcagagcc tttagcttgc ctgcgtgcat |
| 14761 | cgtggctttt gggtaacgga agtagagaag tatgtcattt gactacagag ctctttttta |
| 14821 | tggccatcac ttattatcat cgtgtaggcc agtctgtggg tcactctttg gggatactgc |
| 14881 | cagcgctaag ccttcttgag cctgtgactt cttttaaagc agagccttct ctgcagctcc |
| 14941 | ctccctcagc atctgggcta gtgaacccgt caatgcctgt ggagccatgc tgcctctcac |
| 15001 | atcctcgtta gccaagcatg agtctgtctt cttagactta ctgaacttgg ggtcagtaga |
| 15061 | ctaagactaa gacttaaagt tggggctggg gcaatagttt tggttcataa cacagctctg |
| 15121 | ccctttccta tctcggtgac cttggccagt ctcttgacct ctctgagcct cagtgtcttc |
| 15181 | ctctgtgaaa tgggaattat tattattatt attattttga gatggcatct tgctctatca |
| 15241 | cccaagctgg agtgcaatga cacgatgttg gctcactgca acctcagcct cccgagtagc |
| 15301 | tgggattcca ggcatacacc accacacccg actaattttt gtatttttat ttatttattt |
| 15361 | ttgagataga gtctcgtgct gtcacccagg ctggagcgca atggcacgat cttggctcac |
| 15421 | tgcaacctcc acctcctggc ttcaagcgat tatcttgcct cagcctcctg agtagctggg |
| 15481 | attacaggtg tgtgccacca ccatgcccat ctaatttttt tatttttagt agagacgggg |
| 15541 | tttcaccatg ttggacaggc tagtctcgga ctactgacct caggtgatcc acccaccttg |
| 15601 | acctcccaaa gtgctaggat ttcaggtgtg agccactgcg cccggcctaa aatgggaatt |
| 15661 | attatactta ctttaaaggg atgctgttgc tgggcgcagt ggctcacgcc tgtaatccca |
| 15721 | gcactttggg agtccgagac gggcggatca cgaggtcagg agatcgagac catcctggct |
| 15781 | aacacggtga aaccccatct ctactaaaaa atacaaaaaa ttagccaggc gtggtggcgg |
| 15841 | gcgcctgtag tcccagctac tggggaggct gaggcaggag aatggcatga acctgggagg |
| 15901 | cagagattgc agtgagccga gatggtgcca ctgcactcca gcctgggtga cagagcgaga |
| 15961 | ctctgtctca aaataaataa ataaataaat aaataaataa ataaataaat aataaaatta |
| 16021 | caaaaaaaaa agggatgctg ttttagctca atgaggcaat ccatataaaa agtctgacac |
| 16081 | agaggcagct ctcaccattg gctccctttc ccacaccagc gtgtgggccc caggctccct |
| 16141 | gtgtgagtga gtgcatatgc ctgggtgggg tgggggatgc tcctaactcc ccattacttt |
| 16201 | ttgggtctgt ctcctctccc aggggcccat ccccctcagc agggctccca gaagagggtc |
| 16261 | ccacagctgc ccccaggagc aggagccggg agcttgaggc agtaccctat ctggagggcc |
| 16321 | tgaccacttc cttgtgtggc agctgcaacg aggaccccgg ctctgacccc acctccagcc |
| 16381 | ctgactccgc cacccctgat gataccagca actcgtcctc tgtggtgagc caggagtggg |
| 16441 | agtttggggg acaagagtgg ctcacacctc catctgcagc cctgggtccc acctaaacct |
| 16501 | ccaggccagg gctgagcctc acaccaggga atccgacgag gtcctctgga ccattagctg |
| 16561 | gcagctcggg ccaccccggc gaacccctgg agagtgggga tttctgcaca ggctggccca |
| 16621 | gggcagctgg ggtctggctg tgaaaagaca gggaaaggag tttcagccga caggagctct |
| 16681 | gggtgaatca ccagcaggct cgggctgcca catgctctca gatcccagag tgttgggttt |
| 16741 | ggtgtctagc gcaagagtgg cacagggccc cagggccctg ccctcgacac acagtgctga |
| 16801 | gtcccatggc cagccctgga atagatttcc aagagacaga ggaaagggag caggctgcgg |
| 16861 | gggtcattta accacgaaaa gagaagagcc agcagtcttt cctccagcat ttgcattttg |
| 16921 | aggggctgcc gcagagcaga agagacaggc ttgtcccagg agacccccca aaaggaaaat |
| 16981 | gacagggggt ggtgataaga ctcagaataa aacaacccag cctcacacgc ttcatggcca |
| 17041 | gctccatgct aagggcttgg tgggtgtggg ctcaggggag actcaccaca gcctcagaag |
| 17101 | tggagatttc agtcgcccca tatggggctg aggctcagcg gggggagcaa cttagtggag |
| 17161 | gccacacatc tgggaagtag tggggctagg atttgaaccc acttttctct ggcccctggg |
| 17221 | gctacctcac acagtcccac atgcggaact ctgggactct gggcaaagct cttggcttca |
| 17281 | atggatgtac atgcctccta ggtccgttga agatgagcga cttggcactt agcacctgct |
| 17341 | agagcccagc acagtgccag gcaaacagca catgctttta atttaattga ttgattgaag |
| 17401 | actgggtgcg gtggctcacg cctgtaatcc cagcactttg ggaggccaag gtgggcggat |
| 17461 | cacctgaagt caggagatcg agaccagcct ggccaacatg gtgaaactct gtctctacta |
| 17521 | aaaatacaaa aattagcagg gtgtggtggt gggcacctgt aatcccagct acttgggagg |
| 17581 | ctgagacagg agaatcgctt gaacccggga ggttgcagtg agcttagatt gcaccattgc |
| 17641 | actccagcct gggcaacaag agcaaaactc cgtcttaaat agagaaaaaa aaaaaagttg |
| 17701 | attgattgag acagggtctt gctccattgc ccagactgga atgcaatgat gtgaacatgg |
| 17761 | ctcactacag ccttgacttc ctgggctcaa gtgatccccc cacctcagcc tcctcagtag |
| 17821 | ctgggactat aggcatgcac aaccatgcct agttattatt atttttttta attttgtaga |
| 17881 | gacagggtct cactgtgttg cccagcctgg aagtgctttt tactgaggcc atggagcgga |
| 17941 | gtctacacca cagaaggtgt ggcaccactc actgtcacct cttggggaca gaacttgaca |
| 18001 | gtttaggaag tgctttttat tttttattta attttattta tttttgagat ggagtcttgc |
| 18061 | tctgtcacct gggctggagt gcagtgccac aatctcagct cactgcaacc tctgtctccc |
| 18121 | aggttcaagc gattcgcctg cctcagcctc tcgagtatct gggattacag gcgcccacca |
| 18181 | ctatgcctgg ctagtttttg tatttttaat agagacaggg tttcaccatg ttggccaggc |
| 18241 | tggtttcgaa ctcctgccct caggtgatcc acctgcctca gcctcacaaa gtgttgggat |
| 18301 | tacaggcgtg agccactgcg ccctgcaggg aagtgctttg tttttttttg ttttgttttt |
| 18361 | ctttttgttt tttgagacag agtctcactc tgtcacccag gctggagctc agtggcgcga |
| 18421 | tctccactca atgcaagctc cacctcccag gttcacacca ttctcctgcc tcagcttccc |
| 18481 | aagtagctgg gactacaggt gcccgccacc acgcccagct aattttttgt attttttagt |
| 18541 | agagatgggg tttcaccatg tttcccagga tggtctcgat ctcctgacct cgtgatccac |
| 18601 | ccacctcgtc ctcccaaagt gctgggattg caggcgtgag ccacagcgcc tggccaggga |
| 18661 | agtgcttttt aatgccaatt cccttccctt ccttctgccc ctctcatttg gagagtatat |
| 18721 | gtcctgcagc cttttttctc ccgcaaacat actttctcct ccccttctct ggcaggactg |
| 18781 | ggacactgtt gagaggcagg aggaggaggc ccccagctgg gacgagctcg cagtgatgat |
| 18841 | cccgaggagg cctcgggagg ggccgagagc tgacagctcc caaagggctc cgtctctcct |
| 18901 | caccaggtcc cctgtgggag gagatgctgc aggccagaaa aaggagggtg agtccttctg |
| 18961 | ccaggttggt tcccatggtg atggcctggg gccccccaga tagccatctc actggccatt |
| 19021 | tgggactctg ggcacggctt actttggtgg cctgagtgtt aataatagta acagtttgca |
| 19081 | tgtcatgatg gagcagccac aatgagttat gctacccact ttaattatta ttattattat |
| 19141 | ttgggatgga gtcttgctct gttgcccagg ctagagtgca gtggtgtgat ctcggctcac |
| 19201 | tgcaaccttt gcctccccag ttcaagcgat tctcctgccc cagcctccca agtagctaag |
| 19261 | attacaggct cccgccacca tgcccagcta atttttgtct ttttagtaga catggggttt |
| 19321 | caccatgtta gcccagctgg tctcaaactc ccaaccttca gtgatctgcc catcttggcc |
| 19381 | tcctaaattg ccgggattac aggcgtgatt acaggcacgc ctagcctgtt attattatta |
| 19441 | ttattactat tattattttg agataagagt ctcacactgt tgcccagact ggagtgcagt |
| 19501 | ggtgccctct tggctcactg caacctccgc ctcctgggtt caagagattc tcctgcctca |
| 19561 | gcctcctaag tacctgggat tacaggcacc cgccaccaca cgtggctaat ttgtgtattt |
| 19621 | ttagtagaga cagggtttca ccatgttggc cgggcttgtc tcaaattcct ggctggggat |
| 19681 | tacaggagtg agccactatg cctggcctta gccaaggagt ttggacttct ctaaatagta |
| 19741 | gggagccacc gaaggttttt gaggaggaaa gtcatgtaat taaacctgtg cctcaggaag |
| 19801 | ttccctagat caaagatgca aaggatgaat tggagcaggg aggggttgaa ggatgagaaa |
| 19861 | ccagttaaga gactgttgca ggcactgtag caataactgg ttcaggcatg aatcactggc |
| 19921 | ggatgcaaaa tccctagggt gataatttgt cagcaaacag gatagtcatg gtttcaaagt |
| 19981 | attgtgtccg gaattggtgg gttcttggtc tcactgactt caagaatgaa gcggcagacc |
| 20041 | ctcgcgatat tacagttctt aaaggcggcg tgtctggagt ttgttccttc tgatgttcgg |
| 20101 | atgtgtttgg agtttcttcc ttctggtggg ttcgcggtct cgctggctca ggagtgaagc |
| 20161 | tgcagacctt tgtggtgagt gttacggctc ttaaggagac gcgtccgcag ttgttctttc |
| 20221 | ctccctgtgg gttcatggtc tggatggctt caggagtgaa gctgcagacc ttcgtggtga |
| 20281 | gcgttacagc tcataaaggc agtgtggacc caaagagtga gcagcagcaa gatttattgc |
| 20341 | aaagagcgaa agaacaaagc ttccacagca tggaagagga cccgagcggg ttgccagagc |
| 20401 | tggctggggc agcctgcttt tattctcttg tctggcccca cccacatcct gctgattggt |
| 20461 | ccattttaca gagagccgag tggtctgttt tgacagggcg ctgattggtg catttacaat |
| 20521 | ccctgagctt gacacaaagg ttctccacat ccccactaga ttagctagat acagtgtcca |
| 20581 | cacaaaggtt ctccaagtcc ccaacagagt agctagatac agagtgtgga ttggtgcatt |
| 20641 | cacaaaccct gagctagaca cagggtgctg attggtgtgt ttacaaacct tgagctagag |
| 20701 | acagagtgct gattggtgta tttacaatcc cttagctaga cataaaggtt ctccaagttc |
| 20761 | ccactacact caggagccca gctggcttca cccagtggat ctcgttctgg ggctgcaggt |
| 20821 | ggagctgcct gccagtccca tgccatgcgc ccacactcct cagcccttgg gtggttgatg |
| 20881 | ggactgggcg ctgtggagca ggggcggcgc tcatcgggga ggctcgggct gcacaggagc |
| 20941 | ccacggaggc ggggggaagc tcaggcatgg cgggctgcag gtcccgagcc ctgcccggtg |
| 21001 | ggaaggcagc taaggcccgg cgagaaatcc agcgcagcgc tggtgggcgg gcactgctgg |
| 21061 | gggacccagc acaccctccc cagccgctgg cccaggtgct aagcccctca ttgcccgggg |
| 21121 | ccggcagggc cagccggccg ctccgagtgc ggggcccacc aagcccacgc ccacccggaa |
| 21181 | ctccagctgg cccacaagcg ccgcgcgcag cctcggttcc cgctcgcgcc tctccctcca |
| 21241 | cacctccgtg caagctgagg gagccagctc cggcctcggc cagcccagga aggggctccc |
| 21301 | acagtgcagc ggtggcctga agggctcctc aagtgccgcc aaagtgggag cccaggcaga |
| 21361 | ggaggcgccg agagtgagcg agggctgtga gggccgccag catgctgtca cctctcagta |
| 21421 | tcacccctca gattctgcat cgattactga gagaaaggca cccttaggct ggggagagcg |
| 21481 | ggaagatacc actttcacca agaggctgct cagcatcccc agagtggggc aaaccaacat |
| 21541 | aatatgcccc ctgatatgat gcatggggtc aggggagggg atgctggggt gggcacagaa |
| 21601 | tcccttccct tctaaatcgt cctgctgaaa atgcattgcc tacacctaac cccagtgaaa |
| 21661 | catcagacaa acccgaattg aaagacattc tgcaaaatga ctgggctcct cataggacaa |
| 21721 | aaagcggcta ggggactctt cttgattaaa agagactaac aggatgagga cgtggaatgc |
| 21781 | catgtgtgag cccattggga tctgggacag gaggacacag agctgtgaca ggcattactg |
| 21841 | ggacaactgg ggcttgtttt tttttttttt tttttttgag atggagtctt gctctgttcc |
| 21901 | ccaggctgga gtgcagtggc gcgatctcag ctcattgcaa cctccacctc ctgggttcaa |
| 21961 | gcgattctcc tgcctcagtc tcctgagtag ctgggactat aggtgcctgc caccacgccc |
| 22021 | agctgacttt ttgtatttct agtagagaca gggtttcacc atgttgagcc tgatgggatc |
| 22081 | cgggacacgc ctatagtccc agttactcgg gatgctgagg caggaggatc gcttgaactt |
| 22141 | gggaggtgga ggttgccgtg agctgagatc gccccactgc actcccgcct gggcaacaga |
| 22201 | ataagactcc atctcaaata aataaataaa taaataaata aataaataag ggccatggtt |
| 22261 | gtggggtgag gaggggacag agaaggagag aggtttagga ggcagacttg atggtacttt |
| 22321 | ggacaggagg atacacagtc acagctcctg gcctgggtgt ctgggtagat ggtgtgccct |
| 22381 | ttgctgagag caggtggctg gggagcggat gggcccagcg ctggctgtgc tgtgtgggat |
| 22441 | ttgcgagccc tgaggaagag gttttgaggc ttcagatgct cagggaagca ggggatggag |
| 22501 | atgtggcatc ttcaactgag gagcaatgtt cactcaggca ctcagcagac gtttacttac |
| 22561 | tgcccacaag tcaatcatgg ctgaatagat gaggcagcaa tgagaacagc agcagcatta |
| 22621 | cagcagtgac ctgttttttt gatgttccag gccttgtgct aaataaatgt cttgcacaga |
| 22681 | atcactctgg gctccccgat ccctcactcc tggtctgcct ccttcctcac gggccactcc |
| 22741 | acctcgctct cctttgctga ggccgcctcc tctgcttcac ttttaaatat ctgatgcccc |
| 22801 | acatcccagt cctgggcccc ttctgtcttc tccctgcaca cttagctgct tggctttcgg |
| 22861 | tactgtctgg gaaaaaccat gactccggag tccacatctc tggccttgat ttctcccctg |
| 22921 | aacctgccaa tttatacaac ccagccctct actgcgttaa tcacacctca ggccccacac |
| 22981 | tgcactcact gtttcactca tccccccccg cccttttttt tttttttttt tttttttttg |
| 23041 | agacagagtt tcactcttgt tgcccaggct ggagtacaat ggcgcagtct cggctcactg |
| 23101 | caacctccac ctcccaggtt gaagccattc tcctgcctca ggctcccaag tagctgggat |
| 23161 | tacaggcatg caccaccacg cccggctaat tttgtatttt tagtagagac ggggtttctc |
| 23221 | catgttggtc aggctggtct cgaactcccg acctcaggtg atccgctgat ccgcctgcct |
| 23281 | cggcttccca aagtgctggg attacaggcg tgagtgaccg tgcccggccc actgctacaa |
| 23341 | tactattaac taagctacag acgtgatggg attcaccagt ttttctacta atatcctttt |
| 23401 | tctgttccag gatccattcc aggatcccac attgccttta gtccctctcc tcatctcctc |
| 23461 | cgtctcctcc aatctgtgac aggtttccca tctcttccag tttcccctga aggctggggg |
| 23521 | cacagcgatc tgctcacctt tgactttaaa aaaagtctcc ctggatgaac aatgacattg |
| 23581 | gccccagagt gcccaggctt gggggctact gcaaccatcc aggagagaaa tagtggtggc |
| 23641 | ttggagtagg aagtgggtgt agatgagaga aacaggtgaa tatggaatgt atttatttta |
| 23701 | tttttcgtag agatgggggc tcactatgtt gcccaggctg gtctccaact cctgggctca |
| 23761 | agcaatcctc ctgcctcagc ctcccgaaat gttggaatta caggtgtgag ccactaccag |
| 23821 | gcatgaaacg tattttagaa tgccatggga catcagccac cagcaatttt ttttgttttt |
| 23881 | tgttttctga gacagagtct cgctctgtca cccaggctgg agtgcagtgg ccagatctca |
| 23941 | gctcactgca acctccacct cccgggttca agcaattctc cagcctctgc ctcccgagta |
| 24001 | gctgggatta caggcatgca ctactgcatc cggctaattt tttttttttt tttttgtatt |
| 24061 | tttagtagag atggggtttc accgtgttag cagggctggt atcgatctcc tgaccttgtg |
| 24121 | atccgcccgc ctcggcctcc caaagtgctg ggattacagg catcagccac tgctcccggc |
| 24181 | tgcatccagc taatttttgt atttttagta gagactggtt tcaccatgtt ggccaggctg |
| 24241 | gtctcgaact cctgacctca gatgatccac ccgccttggc ctcccaaagt gttagcatta |
| 24301 | caggcgtgag ccaccgtgcc tggcaacctt gcaaattttg aattccctgc taggtgctgg |
| 24361 | gaactcagac tttcaccaac accagcccag agaggctgga cgaggcagtg gctgcaaagg |
| 24421 | gagaggacag gggatcttaa gaaggcgtga agggagggga tctgcatgaa ggcaactgtg |
| 24481 | gctacttttt tgtttcgttt tttgttttaa gacaatgtct tgctctgttg cccatgctgg |
| 24541 | agggcaatgg cacattcttg gctcactgca acctccacct cctgggctca ggcactcctc |
| 24601 | cggcctcagc ctcctaggta gctgggacta caggcacgta ccaccaggcc ctgctaagtt |
| 24661 | ttaaaaaact tgtagtagag acagggtctc gccatgttgc ctaggctggt ctggaactcc |
| 24721 | tgagctcgag tgatcctccc accttggcct ctcaaagtgc tgggattaca ggtgtaagcc |
| 24781 | accacaccca tagttgtggc tcctttaaga tgaggccatg atacccttta ctcttcacct |
| 24841 | aaagcagtag ccaagggaca tgtacatagg ggtgctcaac acctgcttgg tgaccgaggg |
| 24901 | attgagcggg gtggagggtg acacaccaag gtcatggcaa ctctgtcatg aggctaggag |
| 24961 | ttgcaaagag ggtcactact gaggacagtt acattaatcc tttaggccag tgcttctgaa |
| 25021 | actataaggt gtacaaatca ccagacagtt ttgttaaatg cagatccaga agcagtgcat |
| 25081 | ctgggctggg acctgagatt ctgcatttct ggcaaactcc cagctaccag tttataaaag |
| 25141 | gttgtccatc cctgcacctt tcctatcctc atggcagccc taggagaagc ctcagggtag |
| 25201 | gaacatttgg atacaaaaaa acaggggctt tctagagctg tcgcacagga aacaggctca |
| 25261 | ttagaacctg tctctactaa aaacacaaaa atgagctggg catagtggtg catgcctgta |
| 25321 | atcccaacta cttgggaggc tgaggcagga gaatcatttg aacccaggag gcttaggttg |
| 25381 | cggtgagccg agatcgcgtc attgcactct agcctgggtg acagaacgag actctgtctc |
| 25441 | aaaaaaaaaa aaaaaaaggt cgggcatggt ggctcacgcc tgtaatccca gcactttggg |
| 25501 | aggccgaggt gagcggatca cctgaggtca ggagttcgag accagcctgg ccaacatggt |
| 25561 | gaaaccccgt ctctactaaa aatacaaaaa ttagctgggc atggtggcgc atgcctgtaa |
| 25621 | tcccagctac tcgggaggct gaggcaggag aatggcttga acctgggagg cggaggttgc |
| 25681 | catgagccga gatcacgcca ctgcactcca gcctgggcga cagagcgaga ctctgactca |
| 25741 | aaaacaaaac aaaacaagaa atggggggaa ttccttggat tctgattgaa ggaaatagaa |
| 25801 | ctgggaaggg aagactgtca ggtggctgct gttgggccca cctctccctc cctggggtca |
| 25861 | cacaggccat tgtcaacact tctctccaca aatttgcttc ttctctgtct tgtccgtgga |
| 25921 | tccccagacc ctgtggcctt ccagttcttg aacccatctc ttgagtgagt cagggtctca |
| 25981 | atttcaaatt cttgacctca gcttgagaga ggcatccaag cccttgtcta atcagccttg |
| 26041 | gtcagagagg tggagtcact tggtacagac agtgagacct gtaggaggag ggtgtggggt |
| 26101 | ttgccctaat cactggtcct aagggacaaa gaaaggtaga atatgagagc tgcctggacc |
| 26161 | tctcccctta ccttgagccc ctctcttctc tctccagaca ccggcggtgg gggccggagc |
| 26221 | gcaggacagc actgggcaag gctccgggga gaaagcgggt tgtccctgga gcggcaccgg |
| 26281 | tcaacactga cccaggcttc ctccatgaca ccacacagtg gacctcgaag caccacgtct |
| 26341 | caggcttctc ctgcccaaag ggacactgct caggctgcct ctacacgtga aatccccaga |
| 26401 | gcctcctctc cccatcgaat cacccaaagg gacacctcca gggcctcatc cacccaacag |
| 26461 | gaaatctcca gggcctcatc cacccaacag gaaacctcca gggcctcatc cacccaagag |
| 26521 | gacaccccta gggcctcatc cacccaagag gacaccccca gggcctcatc tacacagtgg |
| 26581 | aacaccccca gagcttcctc tccctcacga agcacccaac tggataaccc cagaacctct |
| 26641 | tctacccagc aggacaaccc ccaaacttct tttcctactt gtactcccca gcgggaaaac |
| 26701 | cccaggacac cctgtgtcca gcaggacgat cccagagcct cctctcccaa cagaaccact |
| 26761 | caacgagaga attccagaac atcctgtgcc cagcgggaca atcccaaagc ctccagaacc |
| 26821 | tcctctccca atagagccac acgagacaac cccagaacat cctgcgccca gcgggacaat |
| 26881 | cccagagcct cctctcccag tagagctaca cgagacaacc ccacaacatc ctgtgcccag |
| 26941 | cgggacaatc ccagagcctc cagaacctcc tctcccaata gagccacacg agacaacccc |
| 27001 | agaacatcct gtgcccagcg ggacaatccc agagcctcct ctcccagtag agctacacga |
| 27061 | gacaacccca caacatcctg tgcccagcgg gacaatccca gagcctccag aacctcctct |
| 27121 | cccaatagag ccacacgaga caaccccaga acatcctgcg cccagcggga caatcccaga |
| 27181 | gcctcctctc ccaatagagc tgcacgagac aaccccacaa catcctgtgc ccagcgggac |
| 27241 | aatcccagag cctccagaac ctcctctccc aatagagcca cacgagacaa ccccagaaca |
| 27301 | tcctgtgccc agcgggacaa tcccagagcc tcctctccca atagagctac acgagacaac |
| 27361 | cccacaacat cctgtgccca gcgggacaat cccagagcct ccagaacctc ctctcccaat |
| 27421 | agagccacac gagataaccc cagaacatcc tgtgcccagc gggacaatcc cagagcctcc |
| 27481 | tctcccaaca gaaccaccca acaagacagc cccagaacat cctgtgcccg acgggacgat |
| 27541 | cccagagcct cctctcctaa cagaaccatc caacaagaga accccagaac atcctgtgcc |
| 27601 | ctacgggaca atcccagagc ctcctctccc agcagaacca tccaacaaga gaaccccaga |
| 27661 | acatcctgtg cccaacggga cgatcccaga gcctcctctc ctaacagaac cacccaacaa |
| 27721 | gagaacccca gaacatcctg tgcccgacgg gacaatccca gagcctcctc tcgcaacaga |
| 27781 | accatccagc gagacaaccc cagaacatcc tgtgcccagc gggacaatcc cagagcctcc |
| 27841 | tctcctaaca gaaccatcca acaagagaac ctcagaacat cctgtacccg acaggacaat |
| 27901 | cccaggacct cctctcccaa tagagccaca cgagacaacc ccagaacatc ctgtgcccag |
| 27961 | cgggacaatc tcagagcctc ctctcccatc agagccaccc aacaggacaa ccccagaact |
| 28021 | tgtattcaac agaacatccc cagatcatct tctacccaac aagacaaccc taaaacctct |
| 28081 | tgtaccaaac gagataacct cagacccact tgtacacagc gggaccgcac acagtccttt |
| 28141 | tcctttcaac gagacaaccc tggaacctcc tcatctcaat gctgcaccca aaaggagaat |
| 28201 | ctgagaccat catctcccca ccgctccact caatggaaca atcccaggaa ttcatctccc |
| 28261 | catcgtacta acaaagacat cccctgggcc tcgtttcccc tccggccaac tcagagtgat |
| 28321 | ggtccccgaa cctcttcccc atctcgctcc aagcaaagcg aggttccctg ggcatccatc |
| 28381 | gccctccggc caacccaagg tgacaggcct cagacatcct ctcccagcag gccagcccag |
| 28441 | catgacccac cccagtcctc ctttggcccc acccagtaca acttgccatc ccgggccacc |
| 28501 | tcttcctccc ataacccagg ccaccagagc acctcccgaa cttcctcacc tgtgtacccc |
| 28561 | gctgcctatg gggctcccct gacctctcct gagccctccc agcctccatg tgctgtgtgc |
| 28621 | attgggcacc gggatgcccc tcgagcctct tcgccccctc gctatttgca gcacgacccc |
| 28681 | ttccccttct tcccagagcc ccgcgcccct gagagtgaac cgccccacca cgagcctccc |
| 28741 | tatataccac ctgctgtgtg cattggacac cgagatgccc cccgggcgtc ctcgcccccc |
| 28801 | cgccacaccc aatttgaccc cttccccttc ctcccagaca catcagatgc cgagcatcag |
| 28861 | tgtcagtccc cccaacacga gccccttcag ctccctgcac ctgtgtgtat tgggtaccga |
| 28921 | gatgcacccc gggcctcctc cccaccacgc caggccccag agccttccct cttattccag |
| 28981 | gacctcccca gggccagcac agagagcctt gtcccttcca tggactctct gcacgagtgc |
| 29041 | ccccacatcc ccacccctgt gtgcattggg caccgggatg caccctcctt ctcatcccca |
| 29101 | ccacgccagg ctcctgagcc atccctcttc ttccaggatc cccctggaac tagtatggag |
| 29161 | agcctggccc cctccactga ctctctgcat ggctccccag tgctgatccc ccaagtgtgc |
| 29221 | atcgggcacc gggatgcacc ccgagcctcc tccccacccc gccacccacc cagtgaccta |
| 29281 | gcgttcctgg caccctcacc ttcaccgggc agctctgggg gctcccgggg ctcagcgcct |
| 29341 | cccggggaga ccaggcacaa cttggagcgg gaggagtaca ctgtgctggc cgacctgccc |
| 29401 | ccacccagga ggctggccca gagacagcca gggccccagg cgcagtgcag cagcgggggc |
| 29461 | cgcacccaca gccctggccg tgcagaggtg gagcgcctct tcgggcaaga gcgcaggtga |
| 29521 | gcccgggggt ggggtcagcc aggtgggctg gggaggaggc tcggcagcag gacaggtgaa |
| 29581 | gggttagggt gccaaaagaa agtgttcaaa tccccctggc ttgccattta ccggctgtgt |
| 29641 | gaccttgggc aaatcgctcc atttctctgt ttctgcatat tgggagtttt ctgttttgtg |
| 29701 | tgtgtgtgtg gttaagtgat gtcatgcaca gcccagtggt taacagcaaa gactgcagtc |
| 29761 | aggagggctg agtccaaatc ctgggtcaaa gccacatgtc aactcgtatg acctgatata |
| 29821 | gtgagttaac ctacctgagc ctcagtttcc tcatctgtaa gggaggggaa catgagcaca |
| 29881 | ttcattcaaa taagggtgaa ctgtcttttt attttttatt ttttgttttt ttttttgagg |
| 29941 | tggagttttg ctcttgttgc ccaggccgga gcgcaatggt gcaatctcgg ctcactgcaa |
| 30001 | cctccacctc ctgggttcaa gcgattctcc tgcctcagcc tcccgagtag ctgggattac |
| 30061 | aggcacgtgc caccacaccc ggctaatttt tttgtatttt tactagagac ggggtttcgc |
| 30121 | catattggcc aggctggtct cgaactcctg acctcaagtg atccacccgc ctcggcctcg |
| 30181 | caaagtgcta ggattacagg agtgagccac cgtgcccggc caaaatgaga gtgaactctc |
| 30241 | aaaccctttg atccagactc atgggttttt tttttttttc ttccgtgcat gagggagaag |
| 30301 | aagaaagtac ccatgatttt ttcctgcctt ggcaaagaaa actcatgggt ccctatgcat |
| 30361 | ttcagaaaac agttatgtca atttaaaatg tatggccagg gccgggcacg gtggctcatg |
| 30421 | cctgtaatcc cagcacttgg ggaggccgag gcgggcggat cacaaggtca ggagatcgac |
| 30481 | agcagcctgg ctaacacggt gaaaccccat ctctactaaa aatacaaaaa attagctggg |
| 30541 | catggtggcg ggcgcctgta gtcccagcaa ctcgggaggc tgaggcagga gaattgcttg |
| 30601 | aacctgggag gcggagcttg cagtgagccg agatcgtacc actgcactcc aacctgggtg |
| 30661 | acagagtgag actccatcta aaaataaaaa taaaataaaa atgtatggcc agagctacct |
| 30721 | attttaagaa agaaagctca cagaattatg gatttttagc agtcaaacat cctgggacac |
| 30781 | ctgaagtcca ggtgtctttt gagtctcctt ggtcagcagc actggccccc tggaagggga |
| 30841 | gcatttcctc ctgatcaggc tgaggtttct ctccccagaa gttccagccc tcgagttttc |
| 30901 | aagaatcaga gctagaattc cattagttag ggtcaagaga acaacagcaa caaaaagcag |
| 30961 | tctggaagtc tcaaaacttt ccatgtatat aattcaacga atttttagtt tatttaccaa |
| 31021 | gttgtccaac cattgccaca atctaacttt agaacatttt catcacccta aagggtatgg |
| 31081 | aatattttgt atctaaaatt cctctttgct gctccttggg aaagttttct cctggatttg |
| 31141 | aatctgcatg agaaacctta tcagtcttgg ctgggtgcag tggctccctc ctgtaatccc |
| 31201 | agcactggag cttgcagtga gccaagatca tgccactaca ctccagcctg ggtgacagag |
| 31261 | tgagactccg tctcaaaaaa aaaaaaaaaa aaaaaaatta gccgggcata gtggcacacg |
| 31321 | cctgtagtcc cagctactca ggaggctgag gcagcagtat tgcctgaatt tgggaggtgg |
| 31381 | agtttgcagt gagctgagat tgcgtcattg cactccagcc tgggcaacaa gagtgaaact |
| 31441 | ccatctcaaa aaaagaaaaa aaagaaactt tatctcttgt tcttcaaatt tccttttcca |
| 31501 | gtaagcttga tcatttaata tccagatgac aaccgtgtgg tctccagcat tagtataatt |
| 31561 | ctttctttgg ccttaaaaac aaaaacccag agcagtttta ggtttacagc aaaattgagc |
| 31621 | agaaagtgca gtgttccatg cacagcctct cccactacca atatcctgta ctagataggt |
| 31681 | atgcttgttt ccagctcctg gcctcaaggg atcctccttt cttggcctct caaagtgctg |
| 31741 | gtgagccact gtactgggca gtagggtgat acatttgtta caatccatga acctacacta |
| 31801 | tgaaatcacc caaagtttag ggttcactct tggtgatgta cagcctatag gcttgaatga |
| 31861 | atgtatgaca agtatccacc attgtaacat catggagtag tttcaccctg aaagtcttca |
| 31921 | gtgtctttaa cttttttttt tgtttggaga tggagtctcg ctctgtcacc caggctggag |
| 31981 | tgcagtagtg caatcttggc tcactgcaac ctctgccttc tgggttcaag cagttctcct |
| 32041 | gcctcagcct cccgaggagc tgggactaca ggagcatgcc gccacatccg gctaattttt |
| 32101 | ttgtattttt agtagcgaca gggtttcacc ctgttgccca ggctagtctt gaactcctga |
| 32161 | gctcaggcaa tctacccacc tcggcctccc aaagtgctgg gattataggc gtgagccacc |
| 32221 | gtgcctggtc tgaccttttt tttttttgaa acagggtctt gctttgtcac ccagcagtag |
| 32281 | tgcagtggcg tgatcatggc tcactgcggc ctcaaactgc caggctcaag cgattctctc |
| 32341 | acctcagcct cctcagtagc tgggactaca ggcgtgtacc actataccca gctaattttt |
| 32401 | ctactttttg tagagatgga gtctcacttt gttgcccagg ttggtctcaa actcctggcc |
| 32461 | tcaagcaatg ctcccacctt ggcctcccaa agtgtctttg acctttttat gattactatt |
| 32521 | tcttgtctaa tgtcaatttt cctaactttg tcttaactaa taactcagca gggacattaa |
| 32581 | ggtctttaag ccaaagttaa ttaattttct ttcctttttt ccaggataat ttcagagaga |
| 32641 | taaggtgttt atattaataa tagaaatgag aaatatgttt acacttttat taaccatttg |
| 32701 | tttttacacg atcactccta tacatttaat tctactctct ttgaaatagc ttatttacct |
| 32761 | tttagggaag ttttcataat tttactgaat aattaaaatt tttcctctta tctggcacct |
| 32821 | tttccaagta gaaaaccatt catcctctga atatggaaaa ttttaaaact gtgcagtgtg |
| 32881 | tgtgtaaggg acagactgga gcttatgagc gggtggctgt aggagtgtgc ttttattttc |
| 32941 | tcctatttcc tcatcgatat gtatttattg agcatctact ctgtccaagc attgtcccga |
| 33001 | gtctggaaaa tttttcttaa agggttgtca catcaaagca ttgggagttt tggcttgatc |
| 33061 | ttaatttttt tttccttaga tgcctttggc atctcttaga gtcacgtagc atcctgtggt |
| 33121 | catttgaatt caaaacctag ccttcactgg atttgctaga cttgatggct ttgctccttt |
| 33181 | aattctctct ggaggtcgaa gaacccatct aagcttccga aggaaacgtt tcctttgtca |
| 33241 | tgcttggtgc ggccacatca accctcggga acaggtggcc ccgaggagac gctgttgctt |
| 33301 | cagcatcgga caagccaggt ctctgttggg gtttcagaat cttcttgagt ccctcactct |
| 33361 | tttctctcag ttctacatgg gggaaaaaag gaaggcgagt tctcaggagg aatgctcatc |
| 33421 | aagcaagctg gagttcgtat ggaaaatgca tccatccgga agagaacaca caagtgcctt |
| 33481 | gcttccctgc accgggtgtt tccactattg tcctcctggt cctccccatt agggaggaac |
| 33541 | agcccactgg gtcacgtctg ggctcttgct tcatcgaagc tcctataccc cagttcgggg |
| 33601 | gaaaattcac tctaaggcct tcagctctca cagagtaagg aaacgcactc acctccaatt |
| 33661 | agattacaaa ttggctgggt gcagtggctc atgcttgtaa tgccaagact ttgggaggtc |
| 33721 | aaggcaggtg gattacctga ggtcaggagt tcaaaatgag accagcctgg ccaacatggt |
| 33781 | gaaaccccgt ctctactaaa aatataaaaa ttagctgggc atggtggtgg acacctgtaa |
| 33841 | tcccagctgc tcagcaggct gaggcaggaa aatcacttaa aaccagaagg cggaggttgc |
| 33901 | agtgagctga gattgcacca ctgcactcca gcctgggtga cagagcgaga ctccatctca |
| 33961 | aaaaaaaaaa aaaaaaaaaa gttacaaaca gcctgcccag ggagccctca agctcagaaa |
| 34021 | cacttcctcc agggaacttt ggttcctctc cgaaccagct gccaataact gctaacaact |
| 34081 | aacattattg tgctttatgg ctctgattct aatgacattg gcaaaagaca tgaaacaaca |
| 34141 | aaaaactcca accccatact ttttttttga gacaggatct tgctctttca cccaggctgg |
| 34201 | aatgcagtgg catgatcatg gctcactgca gcctcaacct cccaggctga agcgatcctc |
| 34261 | ccacctcagc ttcccaagta gctgggacta cagccacgta ccaccatgct cggctcatta |
| 34321 | aaaaaatttt tttttcctag aacaaggtct cattctgttg ccagggctgt ctcaaactcc |
| 34381 | tcgactcaag tgatcctctg acctcagcct ccccaagtgc tggaattaca ggtgtaaaga |
| 34441 | cactgtacct ggcctcccaa ccccatatat atatatatat attttttgag acagggtctc |
| 34501 | actatgtcac ccaggctgga gtgcagtggc gcgatctcag ctcactgcaa cctccacctc |
| 34561 | ctggggtcaa gcgatcctcc tgcctcagcc tcctgagtag ctgggattac aggcacgtgc |
| 34621 | caccacgcct ggctaagttt tgtattttta gtagagattg ggtttcacta tgttgaccag |
| 34681 | gctggtctcg aactcctgac ctcaagtgat ccacccacct cagcctccca aagtgctgtg |
| 34741 | attagaggca tgagccacca cacctgggct aatttttgta tttttagaga ctaggtttca |
| 34801 | ctatgttgtc taggctggtc ttgaactcct gagctcaagc catccgcctg cctcagcctc |
| 34861 | ccaaagttct gggattacag gcgtgaccca ttgcgccctg cccaatccca tacttttgaa |
| 34921 | ggtgtcgttc cagcatggct cctcaggcac aggcccacca cagacagcag ggcctatgga |
| 34981 | ggcctgagca gagtttcctc atacttccct ttttgctgcc tcactctggg tccaccacag |
| 35041 | ggtggccttt ttcaggcttc tcttcctggt tcagttcatc gaccaaagac atggctccat |
| 35101 | ttgctaactg agacccaacc aaaataaaca ttcatggagt ggttgggcct tttgttgtga |
| 35161 | ccccttaggt agaggaaggg agagatatgc ccaggttctg tttggtgttt ctacctgtca |
| 35221 | atagagatac cacgttaatc attaacagcc atgcctccca gttagtaagc aagacaacta |
| 35281 | actggaacta gactttttac acaagttaga gatgatcttc atcggatgcg tcaacaatgc |
| 35341 | ctgaagccca gtgtcttaga aacaacactt gcccagggtc aggcgtggtg gctcatgcct |
| 35401 | gtaatcccag ctacttggga gattaaggca gagaatcact tgaacccggg agatggaagt |
| 35461 | tgcagtgagc cgagatcgcg ccactgcact ccagcctggg tgacagagcg agactccatc |
| 35521 | tcaaaaaaaa aaaaaaaaaa aagacacttg cttggacagt ttatgttggc ctctctgaag |
| 35581 | ttgtcaggga gaatgtttct ttcttctgat cttgctcaga gctcttttgc caacagctag |
| 35641 | gatggcctgg agttttcaag gctaagcgct gggattctgt tggttaacag tgatgggagg |
| 35701 | gaaaaaggaa aagaaatcca aaatttacaa tatggacagg aaaaggcaga aagaatggaa |
| 35761 | cacgggggca cacagcagtc tggctctgtc gtcccacctc gtgggctgcg atcatgatac |
| 35821 | aagtgcttgg cccctggaac ataagaagca ctcaataaat gctttgtcat aatgattatg |
| 35881 | tgtgcccagt gcctggtaca cagtaggtgc tcaataaatc ttaattcccg caaagaccag |
| 35941 | tctaaccttt gtctgggggt ggctcttggg ctgctggtgg ggtggtggtt agtcatagga |
| 36001 | agtactggat gagacaggag agggccatgg ccagggccag atccccctcc ccacccaacc |
| 36061 | gcctctactc cccacttttc tcgtcatgtt cgcaagagcc tcatgaggca gaatagagca |
| 36121 | gtgttaacca tccccatttt tcagaagggg aaactaaggc cctgagagtc atcctcactt |
| 36181 | agggcccttc aacgagcttg cagtgggtcc ctggctgtcc cacatctcct cctgttggag |
| 36241 | gtgggagcag aggggctggg gtgctgggag tgggacagtc cctcagagga gtggctgcat |
| 36301 | ttgctcatag gaagtccgag gcagcggggg ccttccaggc ccaggacgag ggacggtcac |
| 36361 | agcagcccag ccaaggccag agccaacttc tccgaagaca gtccagccct gcccccagca |
| 36421 | ggcaggtgag cactgccagc tgtctggggc cccgcacccc aaggggcggc cactgcctct |
| 36481 | tccctcccgc tagaagccag gcagggggct tggacaggaa ggtcctctat gaaagggtcg |
| 36541 | gagtccaatc atggggatcc agctctcccc tctccccaac ggccagcagc tcttggcaaa |
| 36601 | atgcacaaga ccccacatta atcctttgaa gatagcccct ctcccacgct atccccttcc |
| 36661 | tttactaagc actgctgtct tttttttttt tttttttttt tgagatggag tcttgcactg |
| 36721 | tctcccaggc tggagtgcag tgggacaata ttggctcact gcaacctctg cctcccaggt |
| 36781 | tcaagtgatt cttctacctc agcctcccaa atagctggga ttacaggcgt gtgccaccac |
| 36841 | acccaactaa ttttcgtact tttagtagag acagggtttc accatgttgg ccaggctggt |
| 36901 | cactcctgac gtcaagtgat ccgcccacct tggcctccca aggtgctggg attacaggcg |
| 36961 | tgaaccacgg cacctggcca atactgctgt cttgagcaga tttcagggaa agccttgaag |
| 37021 | caagaatctg gctctaaggg ccctctcttt cagggtaatt tgtatcttta accaaaccca |
| 37081 | gagggctagg cccgcctcag tggaatgggg tattggtggt ggaggttctg gcacctgata |
| 37141 | gcccaagttg gaatcagtat ctttgagagg ggtgagaagc caagaacggg ggcaggtctt |
| 37201 | tgcagatctt tttctggaac tgaggaaagc gggtgaaagg gaggagctga ccccggaggg |
| 37261 | gtggggcaga gaccctgtgt actttttggc ctgctttgat tcaggctgct gtgtggacac |
| 37321 | agccttgact ctctgggggc ctaagaaagg cagagctggc agccaggtcc ccagagagag |
| 37381 | agcctcacct accccctcac ctcatcccca ggtgaccatg ctccctgcca aacaggcaga |
| 37441 | actgacccgg cggagccaag cagagccccc tcatccttgg agtcctgaga agagacctga |
| 37501 | gggagatcgg cagctccagg ggtccccgct gccccccagg acatcagcca ggacccctga |
| 37561 | gagggagctg cggacacaga gacctctgga gagtggccaa gcaggcccaa gacagcctct |
| 37621 | gggggtgtgg cagagtcagg aggaaccgcc agggtcccag ggccctcata gacacctaga |
| 37681 | aaggagctgg agcagccagg agggaggcct gggccctggg ggctggtggg gatgtggaga |
| 37741 | gcccagcctg ggggcagcca aagccccgga gggagcatgg gggggcactt ccagggagta |
| 37801 | caaggagagc tgggggcagc cagaggcctg ggaggagaag cccactcatg agctccccag |
| 37861 | agaactagga aagagaagcc cactcacgag cccccctgag aactggggag gccccgcaga |
| 37921 | gtcctcacaa tcctggcact ctgggacacc cactgctgtg ggctgggggg cagagggagc |
| 37981 | gtgtccatac ccgcgtggct ctgagaggcg acccgagctt gactggaggg atctgcttgg |
| 38041 | ccttctccgg gcaccaggag agggggtctg ggcccgtgtc cccagcctgg actgggaggg |
| 38101 | cctcttggag ctcctgcagg ccaggctgcc ccgcaaggac ccagctggac acagggatga |
| 38161 | cctggccagg gctttagggc cagagctggg tcccccaggc acaaacgatg tccctgagca |
| 38221 | ggagtcacac agccagccag aaggctgggc cgaggccacc ccagtcaatg gacacagccc |
| 38281 | cgcactgcag tcccagagcc cggtccagct gcccagccct gcctgcacct ccacccagtg |
| 38341 | gccaaagatc aaagtgacaa gaggaccagc gaccgcaact ctggcaggcc tggagcagac |
| 38401 | gggccccctg gggagcagga gcactgcgaa gggccccagc ttgccagagc tgcaggtaag |
| 38461 | gaggtttcca ccctcccaag ggtagccaga aagtcagccc cactcagcca agtctccttg |
| 38521 | gaaaagcctc cccttggagt agcctaagct cctgcatccc cctccaggct cagacctcag |
| 38581 | cctccctgct gaccacaacc catcccgatc accaaactgg cccaacaatg atgatgagca |
| 38641 | acacagtttt aagcgtttat gggccaagcc ctgatctgaa tgtttgacgt gtgcttttcc |
| 38701 | catttagtct ccttagcaac caggtgagaa atacactatt tacagataag gaaactgatg |
| 38761 | cgcagggtga agccacttgc tggtggggca tgggcaaaac ccaggtctgc tcagtgcaga |
| 38821 | ccacactgag gcctcaagca agatgctctg gccagggcct attcccctcc tggctccacc |
| 38881 | atacctggtt tatgttgtgt ctggtggcac acacctgacc ccacctggga ctgtagccag |
| 38941 | tgtgggcatg ccccttcccc tggtggtact caggtacctg agtccaggac agcctcgctt |
| 39001 | accctctctg gcccctacag ctcctctcag acacagagaa gagggtgcat caatgctcac |
| 39061 | tgggttcagc tagctgccaa gacaggctgc ttaaatgagg gtcgtgtgca aagtgggccc |
| 39121 | agcgccgggc acacagtagg tgctcgccct tcccctgctt cactggtgct ttggtgctgc |
| 39181 | cttgaagccc agaacattct tcatctgaag atggtgggca gggagggtct tggaggccag |
| 39241 | aatcctgcac cctggcaata gctcattggg aggacggcat gtggttttga acaccagcct |
| 39301 | ctctctctct ctgtaagatg gaccaaccac ttctctgacg ggcagcgctg gggtgccgtg |
| 39361 | ggggttcctg cttgtttctc caaagcctcc tcatctccta gatgcaaagt ccaacgccac |
| 39421 | ctgccccagg atgagcaggg cttccaggca tcagggtgca ccccgtgctg ggcccagggc |
| 39481 | cttgccatat gtgcctcaag tggataggag gcagctgcca cttgacagat ggggaaactg |
| 39541 | aggctcagac tccttaggca tttgctgggg gtcacacatg cataggtcgt gagaaatgag |
| 39601 | atcaaagcat ccactccaaa gtttgtgctt ttatttttat ttatttattt acttattgag |
| 39661 | acggagtttc gctcttgttg cccagactgg agtgcaattg cacaatctca gctcactgca |
| 39721 | acctccgccc cccaggttca agctattctc ctgcctcagc ctcccgagta gctgggatta |
| 39781 | caggcatgtg ccaccatgcc cggctaattt tgtgttttta gtagagatgg ggtttctcca |
| 39841 | tgttggtcag gctggtcttg gactcctgac ttcaggtgat gcatccacct tggcctccca |
| 39901 | aagtgccggg attacaggtg cgagccacca tgcccggcct aagtctgtgc ttttagccac |
| 39961 | cacgtcacaa agctcctgag cccatgggag gactggagaa ccctctcccc tgtatttccc |
| 40021 | tgtggctttg atgagttgac cctccaatca actcaatgcg tggacatcag agaagcaggg |
| 40081 | ataagtcagc tcgtcctgcc ctcagattgc tgtcaggctt gttaggaagg cacacagaca |
| 40141 | gctcaaggag gacacggtgc tgtgtcccgg ggcatgctgg gtgccacagg ccggccaggt |
| 40201 | caagaaggaa gttagaaaga ttcttcagtt tcatggtgaa gctcagggtt ctcaggaggc |
| 40261 | taggagatgg tggctgggaa gggatgggag caccatgtac tgaaaggaat ttcacagatg |
| 40321 | acaaaacgag taaccaccag gtcctgctgg ggctccagct gctcctctct atgaaaatcc |
| 40381 | ttggcttggc acagctgggt gcagtggctc acatctgtaa tcccagcact ttgggaggct |
| 40441 | gaggcaggca aatcacgagc tcaggagatc gacaccagcc tggctaacac ggtggaaccc |
| 40501 | cgtctctact aaaaatacaa aaaattagcc gggcgtggtg gcgggtgcct gtagtcccag |
| 40561 | ctacttggga ggctaagaca ggagaatggt gtgaacccgg gaggtggaag ttgcagtgag |
| 40621 | ccgagatcac gccactgcac tccagcctgg gtgacagagc gagactccat ctcaaaaaaa |
| 40681 | aaaaaaaaaa aaatccttgg catggcactc aaggccggct tcccttcctt ccactccgga |
| 40741 | tttgcctcat tggtgctgag ctggctgcag ggcctgtgag cacatatccc tgcctttgca |
| 40801 | catgctcttc cctgttcagc caccaaacgc ctgttcattg tcgaagtctt aggtcaggct |
| 40861 | ccctgtcctg agaagccttc cctgaacagt cactcctagt aatggtcccc ttctgggctt |
| 40921 | ggcagttttg cttccatttg tccatgcaac atccatctaa tagggttgtc atttccccat |
| 40981 | aagctccaca caggcaggat ctctacctgc tttatctctg taactctagc tctggcccag |
| 41041 | ggcctgacat aaaggagggg acagctcagt ggatgcatgc aagatggagg acacaggggt |
| 41101 | cagtgggtga acaatgaatg aatggagggt gaatgtgtga atggatgatg gatcaatgat |
| 41161 | gcatgttgaa tgaataatag acatcttttg gatggatgac agatgggtga tgatggatgg |
| 41221 | atagctgggt gatgaatata tggtggtggt ggatatatga tggatgttgc ttaggtgatg |
| 41281 | gatggatgaa ctttgggtgg aatatgaatg gatagatgtg gcatgggtgg atgaatgtca |
| 41341 | gatgatagat gttggagata tgggtagatg gatgatggat ggagagaggg atatgtaggt |
| 41401 | attgggtaaa tggatatgtg atggatcaat ggagggtaga tacatgaatg ggtgatagac |
| 41461 | aatggatgat gggtacatga gagggtgagt catgtgatag acactggatg gttggatgga |
| 41521 | tacatggatc tactgatgct ggacgttaga tgggtgttgc atggacagat gatagaatgg |
| 41581 | atggatgagg tggacagatg catgcatcct ggagaataag ttgggctaag ctgtgttctt |
| 41641 | tttttaatct ccagttccaa ccagaggagc ctgaggagtc agaaccaagc agaggccaag |
| 41701 | accccctgac tgaccagaag caggcagact cggtagctgg gtcatgggtg tgggtacata |
| 41761 | cggtggggaa gggaggggga agcaggttgg agatgggctt cattcctcag aagagcccta |
| 41821 | gaatcaacca gaccaaagtt ggcattgcca tggggtcatc tatgtgcatg gtggggcctt |
| 41881 | aagaagaggg gctctgcagg agccaccttc tggcttgggg acctgggtag gtgggcgatc |
| 41941 | ctggggtcag aaccctaggc catgcttctc atttgctggg actgaaaagg tcaatgtctt |
| 42001 | tccctgcgca aatgatggtt tggccccagt ggtctctgtg ggaagagatc cttccggttt |
| 42061 | gaaagggcca ggccaggctg aaagagcagg gcttccacga gcaggagcca gagagctgag |
| 42121 | gatgtaccca gtcagccagg cctggagggt ctccagggtc ctggaggcgg gagagatatg |
| 42181 | tgcgggggtg ggacccacgg atgctgctct gaggcctggc tgcagctggt gagcacaact |
| 42241 | ctgcccgccc ctccccgtga cctaccaaac agactgttct cgtcaccctc agcctccaga |
| 42301 | gaggtgagat cagagttgag ggccgtgggg cagtgggagg tgggggagga ggagacagac |
| 42361 | tgtttggggc tgtgttttcc ggtcctgctc ttcctcccgt tgcctcctga agccaggggt |
| 42421 | ggcagggagc gtccattggg cagcatagcg ccccccaagt agggccctgc cgtgagtcaa |
| 42481 | agctggggga attctgccca aaccctcact gtgcagatga ggaaatggag gcaggggagt |
| 42541 | cgtcccaggt tatacagtca gtggctagct ggactgggac tagaacccag ggcccaggaa |
| 42601 | ccacccagcg ctctctctgc acctccacct ggcacccatg ctgcccccgt ccctgccccg |
| 42661 | atttcttgcg tggcttcgaa cggcagtgct gtaacttcga gcagtccagt cttcccatct |
| 42721 | gtggaatggg cggggacagg ggacactggt cgctaaaggc gctgccagcc ctcccaggga |
| 42781 | ctctgacaac tttgctcttc ctctttctgg gttatttctt catatcctgg agccagagta |
| 42841 | gtgaacgagg cggaaacagc aatgccgggg tgcccctcta gtctccatga aacgggcccg |
| 42901 | gcccatcaac gtggaaaata tctcaaccta cccccacccc taggcagcct tgactggtca |
| 42961 | gaactgataa ccacctcatt catgcattct ttcacacaca cttagtgagc accttctgta |
| 43021 | tacccaggtg tgggtcacag gtgagtcgga gcgccatcaa agagttcagt ctagtgagat |
| 43081 | ggttgcatgc ctgtggtcag gactgtgatg gggaatgtgg tagcagggtc gggggcaccc |
| 43141 | ctacccccaa agccctcctg gacctgtgct ttgatgcttc aagttccact tccaaagagt |
| 43201 | ctgcagattc cagagcgagg accctgggct ctctcctacc ttcccagtaa cccatgtgtg |
| 43261 | gctttggaca agtgcctctg cttcacaggg gccttggttt ccccaacgag gatgactcca |
| 43321 | aagagggagc atttgggggc tctgctagga atgtgtgtag aagtccacaa ctcattccct |
| 43381 | ttgcttaagc tggggtcaca aacctaacgc cctgaaggcc aggcaggtaa caatggtgag |
| 43441 | aaggtggggg ggcaccgagg gcacttgtgt gactcctcgg ttccagatag ctgttgccat |
| 43501 | ggggaagacg ggcccagagt ttccacatct ttcgatattt caagaaacac tggagatatg |
| 43561 | tatttatatg tgaaatctct aacctaatgt gaaatactct gaaaacacta tgtgggcaga |
| 43621 | aacatacctg tggtctgcac cccctccagc tggcgcccgt ggtttgactt tagggtgatg |
| 43681 | acgtgcacac cttcaaaggt gctgagccct gggagccttg tcagtccaac acgatgtagt |
| 43741 | gtgaacaagt taaccagctc gtctcggcag ttctgggagg agagaaagat gggaaccctg |
| 43801 | gggctccatg gtggggggca ccacagaatg ggcaggggag gctgtagcca ggggtgcccc |
| 43861 | catcctgcca aagggacctg gggccaacac tggaccctgt ttgacaggca gacaagaggc |
| 43921 | cagcagaggg caaggctggg agcccgctca agggccgact ggtgacctca tggcggatgc |
| 43981 | ccggggaccg gcccacgctg ttcaatccgt tcctgctgtc tctgggggtc ctcaggtggc |
| 44041 | gaagggtagg ctggctccag tggggactgg aggggtgagg gtggatagag acggggatgg |
| 44101 | gagggaggag ggggctgtta agcaaaggag agagagtggg ggctgaggag gaggcaccta |
| 44161 | gggccgtcgc atgacaggag aggtgggagc agagctctta agctcatcct ggtctcagca |
| 44221 | agcgagtggg tttcccactt ccggggcagg cttgggggct gctttcagct tttggagtga |
| 44281 | ggggtgatgg aataatctac acagctgaaa gcttccaatc agcagcctct cgccagccct |
| 44341 | gggccaacgg ggagccaggt atgggggaag ggaaggcgag cttataagtg gctctgggat |
| 44401 | tgcaaagtgt gtctcagacc tgggcctgag ccagtttcac aaacatctgg cgttacgtaa |
| 44461 | cccatcccct gcccagagag gcctggaaac actcaagcct ctggctgggt tttccacaag |
| 44521 | ggcaggcagc tgagacccgc tccctcccac ccgcttcaca gcaggacaga aggggcctgg |
| 44581 | ccatctccag cagccacagg ctggctcatg gggtctcttc tctctttgag aggctgactc |
| 44641 | tcttactcac cgctgttggc tctgagtcca gcactaggct gggcacctca gagacagtgt |
| 44701 | gcccatttta cagatgaagg gactgaggcc cagggaggtg agctgacttg cctgaggtcg |
| 44761 | cacagcttgg ggtataaagg agctgatttt cctcattctc agatgagaga agggttgtag |
| 44821 | ccaaacttac tgagcctcgg ttgccacatc tgtctattag gaaaaagtct gtggagactc |
| 44881 | agggctgcag agagggctca gggagatcag gatgcatgga ggttgcgggg ctgttgtcga |
| 44941 | aacactaact ttcccatcca cttcagaatt tccttttttt aaaaaattaa gacggagtct |
| 45001 | cgctctatcg cccaggctgg agtgcagtgg caccatctct actcactgca acctctgcct |
| 45061 | cccgggtcca agtgattctc ctgcctcagc ctccagagta gctggaatta taggcgtgcg |
| 45121 | ccactgcacc cggctaattt tttgtatttt tagtacagac agggtttcac catgttggcc |
| 45181 | aggctggtct tgaactcccg acctcaggta atccgcccac cttgacctcc aaagtgctag |
| 45241 | gattattggt gtgagccacc acgccaggcg tttttttttt ttttttttta aattaagata |
| 45301 | gagtctcgtt ctacccccag actggagtgc agtggcacca tctcggctca ctgcaacctc |
| 45361 | tgcctcccag gttcaagcga ttctgccatc tcagcctccc gagtatgtgg tactacaggc |
| 45421 | gtgtgccacc acacctggct aatttttata tttttagtag agatggggtt tcaccacgtt |
| 45481 | ggccaggctg gtttccaact cctgacttca agtgatccgc ccgccctggc ctcccaaagt |
| 45541 | gctgggatta caggcgtgag tcacccagct gagcccagaa tttcctttcg ctgctctttg |
| 45601 | tggttaaggc accgagggtg agcaggcaag catggttatc tgagtagtgt ggtaccttta |
| 45661 | agccatgggc aggaattacc ttgtctaact aaaggtcacc aacacacagc acgtctggca |
| 45721 | ggagggggca ggtaagtcct tggtagagcg aaggaggagc gaagcaggga aagcactctg |
| 45781 | gggtaatgat gctggctaac cagcacttat tgtatgccta ctacatgccc agctctgtgt |
| 45841 | tcactgcatg gcgccaccta attgcatttg atccacactc ccaggtgcag ttagacgcct |
| 45901 | actgtccatt ttacagatga tgaagtgaag ctcagagagg gagggtcact tgctccaggt |
| 45961 | cacacaggga gcaaggggca gaactgagat tcagaccctg ctctggctga cctcagagcc |
| 46021 | cagtgcttcc aacacgcccc ctacaccact gccttcacat cctgatctct ctgagccctc |
| 46081 | ctgcagctct gggagtcccc acagattttc cccattagaa agatgcggca accgaggctt |
| 46141 | ggtgaggttg gctacagtgg ttctctcatg tgggagtgag ggaaatgaag agctagggat |
| 46201 | ggaactgcca gcagggcctc ccagttacac agagcagatt taaacttctc ttgggctgaa |
| 46261 | gcagtgaaat tagcccaagg ccaagagcca gaaagcctgg agtcgagttc tggcttggca |
| 46321 | cctaacttgt gtgcccttct ctgagcctta ccttattatt ctcagtagag taacggtttg |
| 46381 | agactggata acctcgaagt ttggtagctc tggtctttgc cagtttaata actcacccct |
| 46441 | gggtccctga ggctgtgctg tctctgacct gccaggatga gttgtggttt cttcattggt |
| 46501 | gcattcattc attcgagccg tgcttacgga ggatgtgctg tgggcttagg ccctaggctg |
| 46561 | gtttccaaga tgtagacaaa aaacattcca gccctggcca ctggagccca gttaggtaag |
| 46621 | gacactgaca tgtaactgat gagaacagtg tggtcaccac tttagtgggg aaaggtacag |
| 46681 | atgtagggag cactgggcca gcagttccta agtctgctgg ggacactttg gcttcctttg |
| 46741 | gtctgagact tcaaggaaga aattttccag gccaacaagc attgggaggg ggaaggggag |
| 46801 | aaagagagag agaatgtgtg tgtgtgtgtg tgtgtgtgtg tgtgtgtgtg tgtgtgtgct |
| 46861 | gggtgtgtgt gagtgtgtgc tgggtgtgtg tgtgtgtgtg tgctggggta cagaagacag |
| 46921 | gaagagaagt gttccaggca cagcagagaa ccatcagggg gtagaaggac ctgtcttatc |
| 46981 | ctgggaattc ctagtagtcc agtgctgtgt atcttttttt tttttttttt ttctgagatg |
| 47041 | gagtctcgct ctgttgccta ggctggagtg gaatgcagtg gtgtgatctt cactcaccgc |
| 47101 | aacctacgcc tcccaggttc cagcgattct cctgcctcag cctcccaagt agctgggatc |
| 47161 | acagccatgt gccaccacgc ccggctaatt ttttgtattt ttaccagaga cggggtttca |
| 47221 | ccatgttggc cagcctagtc tcgaactcct gacctcaggt gatccacccg cctcggcctc |
| 47281 | ctaaagtgct gagattacag gcataaacca ccgcgcccag cctccagtgc tgtgcatctc |
| 47341 | tttatttact tattgtattt tgcaaaattg accaaagtat gtgtctctcc agcctgtcaa |
| 47401 | atgagaatct ggaaggccag gatgacaggc aggagccagg ttgtgatggt tcctgactgc |
| 47461 | ctagaagagg aatgtagcac tgatctgtca gcattggtga tgtgtggaag aactttgagc |
| 47521 | agggaagtgg tctggtcact gtgttgcggg tactaggagg agtcgcaggg gccagaaaag |
| 47581 | cctggaggag gctggtgctg ttaggtgccc agggcactgg accaggaccc tggtggtagg |
| 47641 | tgaggtctcc tgcgtgttca ttcactctcc caaggactga ctgcctgagt gaggtgcaag |
| 47701 | ggtagggtga gaaaactcag cccggggtgc cagaaggcca gtttctagag ttagatggac |
| 47761 | ttaaccttgg cctcattcct catggtggag agactcctgg cagcaaatgc tgggaaagaa |
| 47821 | cttcagctcc ctgggcctcc atttctttat ctgcaaaatg ggaacagtaa ttataaaagc |
| 47881 | aataattttt ttttttgaga gggagtctcg ctccgtctcc caaggtggag tgcagtggca |
| 47941 | caatctctgc tcactgtaac ctctgcctcc cgggttcaag cgattctcct gcctcagcct |
| 48001 | tttacatagc tggattacag gcgcctgaca ccacgcccgg ctaatttttg tatttttagt |
| 48061 | agagacgggg tttcaccatg ttggccagtc tgctcttgaa ctcctgatct caatgatcca |
| 48121 | cccacctcag cctcccaaag tgttgggatt acagacctga accaccgcgc ccggcaaggg |
| 48181 | caataatttt tgaaccccaa atcaagatga cagaataatt gaaatgccaa ttattccaag |
| 48241 | cttccctgcc tccttgccta aggatagcta atgagggata aagaggttgt cctaggtgcc |
| 48301 | ttacttgaat taacacttaa tccttccagc aacctcatga ggtaacagca ttaccccatt |
| 48361 | ttacagagag tgaaattgag caccgagagg tgccttgctt aaggtaacac taggctggaa |
| 48421 | tccccgtgcc acacgaactt cctagtgcat ttagcaacca attccaccac ggtccctact |
| 48481 | cacctgcagg aaaagagcat ctaacaaacg gcccagcgtt ctgggaacct gagcaacggg |
| 48541 | tgcacgccca cacctgaaaa accccaaacc aaaaaagacc catttgtgag gagtgggcat |
| 48601 | ttgggagaga ccctgccatc caagaactca ctttgtgtcc agcttactcg ctaggaattc |
| 48661 | cgcctcttcc tgtgtgacct cgggcaaatc ggctcacccc tctgagcctc cgttttctca |
| 48721 | ttagcaaaat aagacctccc tcccaagtct gtggtgagga cgaaaatcga ctcttggcga |
| 48781 | gtgggacccg ctgagtcgtt agtactgggt gctttgcccc ggtccccgcc aaatcacggc |
| 48841 | gccgagccct agcggaccca tcgctccggg gccgggtcgg gaagcgcagc ctgggcgagt |
| 48901 | cgtgcccacg cccggccgcc cgcatcctcc gcccccgcgc cattgccgga gtcacctgca |
| 48961 | ccctgggccc ttccttcctc ccagcgcgcc cgcggcccgg gcccgcccgc cctccggccg |
| 49021 | ccaccgccct cccttccctg cggccccatc cgcccacccc gccgtcccgc cgtcccgccg |
| 49081 | ccccgccgcc ctagcgcgcc cgtcccgttg cggcaggtcc cgcccccggc ccgtctcgcg |
| 49141 | cttccattgg cccgccgcgg cccccgccgc tgtttgtctc ggcttccccg ccacccattg |
| 49201 | gcccgctcgg gtcctcccag gaagtttgaa aaaaaaaaaa aaaaagtttt atgggcggat |
| 49261 | ggaaggggcc ggggcagcgt cggggaaagg aagggccgga ggcgcggcgg cgggcggccg |
| 49321 | agaggggcgg cggcggcggc ggcggcgggg ttcccgcgcc gcggagcccg gcccgagagc |
| 49381 | cgcgtccacg ttcctgcctc ctgctcccgc cgccctgggg cgccgccatg acggtgagtg |
| 49441 | ccccagtcag ccgcccgcgc ccgaggccgg gagaagggcg acgacccgag gcagagccca |
| 49501 | gcctctcccc tccggggcag ccccctcctc atttcccgaa ggcgagagga gggctgggcc |
| 49561 | ggaagacggg gccacctccg aagcgctttc ggccagaccg gcctcaccct tcccgcaggc |
| 49621 | tcctcctcgc cttccctcgc ccactcgccc ccgggagccc aggcgaccct gaggggacgg |
| 49681 | aacgacccgt ctcgggcgcc ccctcctcac acttgcggca ccagtcacgg gtggattcgg |
| 49741 | ggcgaccctt tcccacctcc cctccagccc cctagacgaa gacccgattc cccaaggcgg |
| 49801 | ccaggggccc cagggaggag cggaggtggg cctgggctgg ggaggggcgc tcgtgggtgc |
| 49861 | ggatgggtcg cgctgacccg ggcctgggca agccagccct ccgtgtggcc gctggcccga |
| 49921 | ggtcgggtgg gcgagaagtg gccggaccgc cccacccaca gcccgggagg cagtcccggg |
| 49981 | acgtctgaag cagcgggata ccacgtcttc ctgggcttgc actttgcagc ggtcctgggg |
| 50041 | ccttgggaca agcctgcgag tctgagtagg gggcatgatg gcaggggttt gcctttctca |
| 50101 | tgggggtgag ctgtacccaa gccccatcaa ttacctgtgc catagttagc atattgagcc |
| 50161 | tcacagctgg gttcgctgga ctccccactt cacagccaga taaactgagg cttggggagg |
| 50221 | ggcgctggtg agttgcccga ggtcacccca agtataagag aaggagtagc tcctttccct |
| 50281 | gtccttctgg cccactgacc gtgatgttcg ggaagggagg ggtggggctg cttttggttt |
| 50341 | ctggctagtg cttcagggag gctctgacag cccagtggcc ctcctggggg aggatgagca |
| 50401 | gggtgtgtgt gtgcacaggc ggcagtaggc ctctttcagg ggagtggaga ccctgcggag |
| 50461 | gagtgggtgg gctgctgggg gcgggctggg ctgctggggg gcgtggcacg cctagcccct |
| 50521 | gccctgtgag tgtgatggcc tggacttcct cggcgtcctc ctggacatcc tgccacctcc |
| 50581 | ctgctggccc ttcctgtcgt cagcagcctc cccttttctg tcaggagcag aagggatgtg |
| 50641 | gggggaggag ctgagtcagt gtcctggggc caggaggaga gaagttgcct tgcccaccgg |
| 50701 | gtgcctgccc ggccctcagc cgggtgcttt cacgttacca gatttgatcc ttgaaggttg |
| 50761 | gtagtaacat cttaatcaaa cagatgagga aaccaaggtg tggcgaggtg aagtgtcatg |
| 50821 | ccccaggtaa ctcagtgagt cccaggctaa gccagaattt gtatccaggt ccgttccagc |
| 50881 | tttctttcac agttgcctgt cctgggggca gcacgtgctg agcaagggta aggctgccgg |
| 50941 | aagcagcgtg tggggtgctt ggaagatgga cagcacatcc ctgctggtgg cagcagcctt |
| 51001 | cctgagggag gtgtcctcct gtgattatag ggccttgtca ggtggagatg gactagcgag |
| 51061 | gagacagcag tgtggacaga aacgggtggt catgtttgag aagtagcaag agcgtaggga |
| 51121 | aggggtctgg ggaggagggc ctcactgcta gggcttggac aggggcaata gagagccatg |
| 51181 | aaagctttta gagcagagag gaggctatga tcaccattaa tagtgttcat tgagttcatc |
| 51241 | ctggctgcca tgcagacctc agcctgctct cagctggtgc tcctcctgta cccacctcac |
| 51301 | accctcctcc cctggccccc gtgccctgaa tcagggatcc atacagagca agtggcacag |
| 51361 | gtgtgtgagt gtgtgaggct ccggggcttc agggagttgg gattctcttc cacccctttt |
| 51421 | gcctttgcct gggtgcagct ttgctcccgg atcagccttc cccctcgcct gctgggcatc |
| 51481 | aaattatatt tggtcccttg tttactgtct ctttcctaca gtccaccagg gcaggggcca |
| 51541 | cagctcttgg tcacagctaa tttcctggcc tagaacagtg cctgataact ttaaaaaaaa |
| 51601 | attttttttt ctgcaaacac tccctgagcc cctatctgtg ctgggtcctg gcaggtaaac |
| 51661 | agtgtatgtc cctgactgtg agttgcatct aatctagcct gtgaaggata agtaaagggc |
| 51721 | ctcaagatag tcacggatgc aacagaggcc tctgagaggg gccatgggag ggcaggacgg |
| 51781 | gggcggctgg tggtgcggtg agtggcatga actgtgaagg gacctcagag gtggccatta |
| 51841 | agctgggtct taaggcctga gtagccgctt gcctagtcat cggaggagga ggaagcatag |
| 51901 | tgtttcaggc caaggacatg gcacatatga agggcccgga gtcctggtgt gttgtgatcg |
| 51961 | gcaaggaata tgcagtggta taaacagggg cccctcagca aggggaggcc aggctggaag |
| 52021 | agtgtgatga ggcttccctg ccatcctgag gcagctggac tttgttttaa agccacctgg |
| 52081 | agaccgggtg tagtggctta tgtgtgcctg taatcccagc attttgggag gctgaggtgg |
| 52141 | atggatcact tgaggtcagg agttcgagac cagcctggcc agcatggtga aatcccatct |
| 52201 | ctactaaaaa taaaaaaatt tgctggacat ggtggtgggt gcttgtaatc tcaggtattt |
| 52261 | gggaggctga ggcaggagaa acgcttcaac ccgggaggtg gaggttgcag taagccgaga |
| 52321 | tggcgccatt gcactccagc ctgggcagca gagactctgt ctcaaaaata aagcaagcca |
| 52381 | cctggagccg tccctggaac tgttaagcaa gcctgggata ccaagccttt ttgagtgagt |
| 52441 | cccgccagcc cgagggtgtg tagaggggtt gtgtgcctgg ccaggtagga ggcggggctg |
| 52501 | tgagaacctg ccagtactcc cgaaaggcgg aaggagaaac tgagcttcta gcaggtgaag |
| 52561 | aagaggtagc tgggcctgaa gagccctgta tgaaggggca tcgtgggaga tttagatgat |
| 52621 | cggacctttt gaccctgggg acaggtttcc ctgccagttt aaaaactcac attggttggc |
| 52681 | cgggcacatt ggctcacacc tataatccca gcatttttgg aggccgaggt gagcggatca |
| 52741 | cttgagctca ggagtttgaa accaacctgg gaaacatagg gagaccccat ctctacaaaa |
| 52801 | aaaaaaaaaa aaaaaaaatt agctgagcat ggtggagtgc ttatgtcgtc ccggctactt |
| 52861 | ggaagctgag gtggaaggat cgcttgagcc caggaggttg agtcagcagt aagctgtgat |
| 52921 | cacaccacca cactccagtc tgggcagcag agcagggccc tgtctcaaca aaacaaaact |
| 52981 | caaattggtg accttctatc cccaaggtca ctgtgtcatg gcgtgcctga catggtatgt |
| 53041 | gatgtgtctg tactgggctc tggaggatca ctaggtttct ccttgtacag acacgttcag |
| 53101 | tcctgtcgga agctgcggga ggggaggacc gccccgtcat ttgccacatc aggcagccag |
| 53161 | gactcagaga ggggatgtga cttaggaagg tcacagtgac tccacttccc aggcagccct |
| 53221 | agaggttcca gcctgacagc ttgtcattcc tgccccaatc tcccagccct ggcccaggga |
| 53281 | ctttcgcctg ggactgccca gtggtgccag ctctgtgggc ccaggaagcc tggctcctcc |
| 53341 | gggctaggtg ggggtagggg agatggccca aaaagcacag aaatttagac cagggggtag |
| 53401 | atatttcctc agcaagtttt tcctgagcct tgctgtggtc aagccccctg caaggccctg |
| 53461 | gggccacagg aggtcccaga ctgaggggaa gtggagccag gtgtggtcac caatcttcat |
| 53521 | tcttagcttg ggatcgccct gggcagcctc cgtctcaggt ggaagacggg gtgggcagaa |
| 53581 | tacagagcag cgggacaaac gcagccccag gggttctcgg ccgtgagcag agcctgtgtc |
| 53641 | tctcccttag acaggtatac agggaggggc tgggatgcca acctactggg tcccccgact |
| 53701 | gtggctgtgg aaggttctgg atcttcagct gtcccaagcc ccggttggtc cacttcctat |
| 53761 | acctgagaag gggctccgtg ggtggagagt gggtcgggag aggtggccag gcagggtggc |
| 53821 | tgtggtttcc agctgccact gtcaccgccc actccactga gccatgccac ccgttctctg |
| 53881 | caggcaacca gacaccttgg cctggtgagg cctcgatgta ggggctgata ggtaggctca |
| 53941 | gagccatggc aaccggtggc cacctcctcc ctgccggcgc caggagcgga gactgcagag |
| 54001 | ggaccgggac ttagatgaga gaggtctccc cagcaggaag ggagggtctg gtggttggca |
| 54061 | cccactagat gagcatggtc agaaaaatat caggatgagc aacttcttta tctaggcctg |
| 54121 | ggtgggggtg cctggcacag gtctccatgg gacatgctgc agctggtagc ccccagaccc |
| 54181 | cggggctgtg cccccctggg cggcacccag aaggtagggc ttgccaggac tgacccttcc |
| 54241 | caaagagatc taggcgggga ccagagggtt tctgacccca gcctgtgact ggctccacct |
| 54301 | cccatagtct cctggagccg aggaaaggac aggactggcg ggcccatggg agggtccgtg |
| 54361 | gggcacttgg tcctcgtcag agggaggaac tggacagttc gcgcagtgct gctggtctgg |
| 54421 | actgccctct gagctctcag ggggtctcta tgcaggcaga gtcctcccag cccctctctc |
| 54481 | ccattcttgg gcgaggggtc ggctggagag aggcccctgg gacatctggg tgtgtgtgcc |
| 54541 | agccacccag cccaacttct tggctctctg agtgccacta tcaggaaagg ggccagggct |
| 54601 | gcctcagtgg ctgggaggcc tgcttggact tgggggttac ctagggcctt agccctcttg |
| 54661 | gctggcttcc caggagctcg gtcccacagg acttggggac agggtgggtg cagcacgctc |
| 54721 | ccctcaccag cccccattgg cttcctgctg gacccaactc acctccctcc tcatctcccc |
| 54781 | acagcccgat ctgctcaact tcaagaaggg atggatgtcg atcttggacg agcctggaga |
| 54841 | ggtaagagga ctgaggccgg aggggaggaa gctggcttgt gctgctgctg ggcccctggg |
| 54901 | cagcccagga gtgggggtgg ggctggggct ggtgtgagaa ccctgggggt ggggctggga |
| 54961 | ggggtggagg ctgcccatta ggcttctgcc tcctggagca gtctgcagtg tgtaaaaggg |
| 55021 | atcacagcaa ggggccgtct agcccctcac acccagtgcc ccatcccttg atctcatcaa |
| 55081 | gagtctcttg gccctgccac ctcagtagga gcctgggtgg gagtcaaggg tcttgggtgg |
| 55141 | aagggctcgt agaagctgtc tggcccgaga cacagattcc taaagcctgg tctgaggggg |
| 55201 | gtggggctca gggcgcaggt gctggggttt cacccacctg gatccccact ggttgagtca |
| 55261 | aagcctcctc tagtcctcag tggccacgtc tgtgacatgg gcataatgga taatggtgat |
| 55321 | tctttctaga gtggataaca aggtcagaga gatcaagcac gtacaggtgc cacaaagctc |
| 55381 | ctggcactta ctgtgggctc agctaaagac ccttcccttc ctctggcata tgtgtccttg |
| 55441 | gaaccagcag ggacagtggc tgtttcctca gctctgttct ttcggctgcc aggctggctc |
| 55501 | cctgcccaca ctgcctgggc aggagcgtct ccccacccct gccctgcatg cgcctgctgc |
| 55561 | tctggtctgc gtgcagcctg catgccggag agggctttga gccagagggg atttcattgt |
| 55621 | gtttccggcc cacatgtttt attccctcca gcccacagcc ttccctaccc gctgcctgcc |
| 55681 | cgctgtccct ggtggctgct ctctaggctc acacaaagcc cggcaccccg ccagtgaccc |
| 55741 | cttcctggcc tccccaccca gggtccccgg agggcagagg gattacaggc cccacccaga |
| 55801 | gctggccatt agtcatcctc tatcacaggg ttctggaatg tccgaactgg aagggccctg |
| 55861 | ggaattcagc cagcccaacc ccttggtgca cagacaggag acgggcccat gagggtgttg |
| 55921 | gtctcttcca ggcctaggag cacctggggc agacccaggc tcagaactca gcgcacctga |
| 55981 | ggcccatgga gctccaccca gcttccatct cagacagtca taggctaggc ctggaagagc |
| 56041 | taaagaaacc tcttggcata gaacccctgt cctactttac agagaggcaa ttgaggtctg |
| 56101 | cagcagcaac tccgtttact tagtgagagg aaaatgcttc taagaggcct ggcctcccag |
| 56161 | aacaaaccca gcaacctggt tggtgtccat ccctatgcca agggccatga cagccatcca |
| 56221 | cacaagagag gatgggcgcc gctcgcctca ccagaaggtc taggcacttc tttttttgtt |
| 56281 | ttttattttt gttttttttt ttctgaggca gagtttcact cttgttgccc aggctggagt |
| 56341 | gcagtggcgc aacctcggct cactgtgtct tccgcctccc gggttcaagc gattctcctg |
| 56401 | cctcagcctc ccgagtagcc gggattacag gcatgcgcca ccatgcccat ctaatttttt |
| 56461 | tttgtatttt tagtagagac agggtttctc catgttggtc aggctggtct cgaactcctg |
| 56521 | acctcagatg atccaccctt ctcagcctcc caaagtgctg ggattatagg cgcgagctac |
| 56581 | cgcgcccagc tatagggact tcttagtagc cctactttat ggttcagaag actgaagctc |
| 56641 | agaaaagtgg agacacttgc ctaaaggcac acagagagaa agggaactct tggatacaaa |
| 56701 | actccttttc tcagggaggc aatgtggaaa gaagcagaag gtatcgtcgt tgcatgcccc |
| 56761 | ttgagaactt accatcttct tgaggagaga aaactaagta cttgaaacaa ccaacagcac |
| 56821 | agacagggct tggtgatgtg agggaaccct gggagggctg gggaccgggt gatatgcact |
| 56881 | gaccctggca gtgggcgggc cgcgactggc attccagtgg agtgaccaga agcccagagg |
| 56941 | tgggctgggc ttggcatact atcaggctgt tctggcgccc ccaccccttt cctacatgtc |
| 57001 | ctgccaatct aaccccagct ggagcagaga caggcactgc aggaaggcac ccaatcacaa |
| 57061 | atcctaaact tacagcttag ggcgctctca cctgctgcgg atgtgggtag ccagcctaca |
| 57121 | gttcaagaag cagaccgtgc atctgggcgc ggtggctcac gcctgtaatc ccagcacttt |
| 57181 | gggaggccaa ggcaggtgaa tcacctgagg ccaggagttt gagaccagcc tggctgacat |
| 57241 | gatgaaaccc tatctctact aaaaatacaa gaaattagct gagcatgatg gcgggcgcct |
| 57301 | gtaatcccag ctactcggga ggctaagaca ggagaatcac ttgaactcag gaggaggggg |
| 57361 | aggttgcagt gagtggagat tgcaccattg cactccagcc tgggcaacaa gagcaaaact |
| 57421 | cctccatctc aaaaaaaaaa aaaaaaaaag gcagcagatt gtggctggca ctcactccta |
| 57481 | ctccagggct ccatccagtc ctacttccca ggggaccatt gtccctgacc atggtgatga |
| 57541 | tctgggggaa tatggaaaaa gacgtaacgt cctggccccg ctgagtccct gacttgctta |
| 57601 | cacaggtcat cacccctccc tgtctgcact gggtccgggg tgcctgagga gccttggcaa |
| 57661 | tcttaaaagg agctccaaga aggaaaggga gggtgtggtt acggagggga aaccaatcca |
| 57721 | tttcacagat gctacccctg aggctcagag aggaaatgga ggggccaggt ctcaaattgg |
| 57781 | cattttccgc ctccccaccc ctcctgtgtg cagcatagtg agtttggcag ccacacagga |
| 57841 | cacctgtaca atcacacaca ctggctcttt cattccatcc tccttgcagc ctccctcccc |
| 57901 | ctcgctcacc accacctcta cttcgcagtg gaagaaacat tggtttgtgc tgacagattc |
| 57961 | aagtctcaaa tattacagag actccactgc tgaggaggtg aggccatggg tgtactgatg |
| 58021 | aacccccgga agggcctggg gtggcctggc tgggttcact ggggtgggtc acaccagggc |
| 58081 | ccacacggac catagtgggc cctcttgctc caggcagatg agctggatgg tgagatcgac |
| 58141 | ctgcgttcct gcacggatgt cactgagtac gcggtgcagc gcaactatgg cttccagatc |
| 58201 | cacgtgaggc tgtgtgcagc ttgggggctg gtggtgggca gcatggctgg aggtccctgg |
| 58261 | gggaggtttt ggtgttgaac atggctcacc atggagactg gatcccttca ctcatttacc |
| 58321 | cagaggccct atcttcagag ccagagctgt gatgccaaca cttagcatat ctgggggaga |
| 58381 | cgctggagcc agcagggctc ttcccaggcc aatggaaggg aggttttgta ccccctgccc |
| 58441 | accccagact caagacctta gatgcatcct gggaaggaag ggccaccctc cctggggtcc |
| 58501 | atagtgggga gggagtcatg cggctggcca tgtggcaacc tacccatgcg gtggccttgc |
| 58561 | agaccaagga tgctgtctat accttgtcgg ccatgacctc aggcatccgg cggaactgga |
| 58621 | tcgaggctct gagaaagacc gtacgtccaa cttcagcccc agatgtcacc aagtacgtac |
| 58681 | taagctggac tggggccttg agggaggcac ttacccttgc gtccccgagt gggtttctag |
| 58741 | gtactcacct gagggctgca cctttctggg ccctcgtttc tctgtcaaca acatgggaag |
| 58801 | agagtagtga gcattctcgg tatcatccag aagtacactg agggtttgag gtttgatctg |
| 58861 | tcttctcaaa tgtgatgggg cctcttctct ttggaggaga tgcctgtgag tcaaaagccc |
| 58921 | ctgccaggtg gctgttctgt ctctgtgtct gtgccgtggg ggccgtgtct aagcctgtgt |
| 58981 | taggaagggg tggttaggta gttcagcctt ggcgcccaca cctcccggga tggggagctc |
| 59041 | actccttctg gaggctgcca cgggcactgg ggcaggaggg ctgagttacc gagtggcaga |
| 59101 | gccccccaca gtttataccc agatgcccat ggcgggctgg gacttggagc tgctgggcag |
| 59161 | cgggaacaga gtgtggtatt aaagggggct gggctgggtg tagtggctca cacctgtaat |
| 59221 | cccagcgatt tgggaggctg agatgggagg atcacttgag ctcaggagtt caagaccagc |
| 59281 | ctgggcaata tagtgagacc tcgtccctac aaaaaattta aaaatcagct ggccgtggta |
| 59341 | gggtgcgcct gtcagctgag gtgggaggat cacctgagcc cggaggccga gtcccccaga |
| 59401 | ctggccacac cgcggctatt cagccttagg ccagaggctt tcctgtctag gtttcagagt |
| 59461 | ccccatctgt cacgttagca tgtcatgtct gtacatgtgc caggatgtgc ccagagcact |
| 59521 | ttgtgaactc tgaagcactg tgcagttgga ctgttattta gcatctaggc ccagctgctt |
| 59581 | taagaaagaa accccgggca cagtaacttc atcgagatag atgttcatgt ctcccttcag |
| 59641 | aacagccccg aggtatgcag tcctggcctg gtggggctcg ctctgccgtt tcaggggagc |
| 59701 | cacctgccac acccactcgg cagcctatag tcacatggct acaagtagct gtgggaatac |
| 59761 | tgcgagtgta gtttttggca gggtgaccac gtacccagct atgactgtgg ataaagagaa |
| 59821 | ttgccatagt tgttcagagt ctctgcaact tgtgttctgt gcaccccgag gtcccgcctt |
| 59881 | caccccatca gcagtgactg ggcatccccc ttgccactgt gagcctcggg aacctgggac |
| 59941 | attggtctcc tacatcacct cattgatcac catttgcaaa aagccccctg cctgctttcc |
| 60001 | acgttcgccc accctgtcct cacatccatc ctgtgagagg cgtcgctgtc ccacattaca |
| 60061 | gatgaggtca ctgaagctca aagagggcaa agagaccagt gtggtggttc acgcctgtaa |
| 60121 | tcccagagct ttgggaggct gaggtgggag gagcacgtga gcccaggagt tcgagaccaa |
| 60181 | cctgggcaat atattgagac cccatctcta tcaaaaattt aaaaaattag ctgggcgtgg |
| 60241 | tggtgcatgt ctgtagtccc agctactcca gaggccgagg caggaggatc acttgagcta |
| 60301 | aggagtttaa ggctgcagtg aactatgatc acaccactct acttcagcct gggcagcaga |
| 60361 | gtgagaccct gtcccttgaa aaacaaaaac aagaacgggg gcgagggaag ccgggcatcc |
| 60421 | tggtgggtcc ctgggctgtc aagaggtgga gctgggatgt tcctggggtc tgttaaccag |
| 60481 | gaagctcagg tcgggctgct tccttccctg gggtctgtct ccagcccctg gcacagggcc |
| 60541 | tggcacacag gtgtcgctcc aaaagtgctc attgtgggac tgcaggggtg aggacccacc |
| 60601 | tgacgtggct ctgctggtgc cctaggctct cggactctaa caaggagaac gcgctgcaca |
| 60661 | gctacagcac ccagaagggc cccctgaagg caggggagca gcgggcgggc tctgaggtca |
| 60721 | tcagccgggg tggccctcgg aaggcggacg ggcagcgtca ggccttggac tacgtggagc |
| 60781 | tctcgccgct gacccaggct tccccgcagc gggcccgcac cccagcccgc actcctgacc |
| 60841 | gcctggccaa gcaggaggag ctggagcggg acctggccca gcgctccgag gagcggcgca |
| 60901 | agtggtttga ggccacagac agcaggaccc cagaggtgcc tgctggtgag gggccgcgcc |
| 60961 | ggggcctggg tgcccccctg actgaggacc agcaaaaccg gcttagtgag gagatcgaga |
| 61021 | agaagtggca ggagctggag aagctgcccc tgcgggagaa taagcgggtg cccctcactg |
| 61081 | ccctgctcaa ccaaagccgc ggagagcgcc gagggccccc aagtgacggc cacgaggcac |
| 61141 | tggagaagga ggtaggcacc acggctggct ctctaggagg ccccttgccc cagcgcccct |
| 61201 | ccagtccctg cattcatgcc ctggcatttg tcttgcactc ctacagtgat ggggagctca |
| 61261 | cccctgatct gctgtgagtg tgcaccccac actcctgcga actagaatct tcttccttag |
| 61321 | accttagtag tttggtttgg ttttgttttg ttttttccta gttctgccct tagcagccat |
| 61381 | agagaagaga agtgggaaga gaagggagag acctgccagg ctcacacctg taatcctaac |
| 61441 | attttgggag gccaaggtgg gagggtccct tgaagccaag agtttgagac cagtctgtgc |
| 61501 | gacatggcaa aaccccatgt ctacaaaaaa atacaaaatt tagctgggcg tgctggtgca |
| 61561 | tacctggagc cccagctact tgagaggctg aggtgggagg atggctggag cccgggaggc |
| 61621 | agaggatgca gtgagccaag atcaagccac tgcactccag cagtctggca atagagcccg |
| 61681 | actttgtctc agattaaaaa aaaaaaagaa gaagaagaag ggcgagaccg tttattgttc |
| 61741 | atcttctgcc cgtggcctcc ctgcctgcac tgactcgtgg aatcctcaca gcggcctcta |
| 61801 | cccctagggt ggacttcagc agttttactg acgggaaaac tgaggctcaa agatgtttcc |
| 61861 | tctgtcacct ccatgatgtg taaacatctg tgaccacagt ttccttttcc tatccctgtc |
| 61921 | tagccttctc tgggccaagg ctctcccagg tcattgacct ggccatccag gaaacagccc |
| 61981 | agcgtgtccc ctggctacat ctcactccag ggctgcttgc ccttctcaaa ccctgaggca |
| 62041 | ttcctaagcc ttccagggac gctgggcttg tatccgtgtg gagctggaag cctgcgggct |
| 62101 | cctcactgcc ttccaccagc cctgccccag cttccaggtc ccactgacca cccttctccc |
| 62161 | tcgtaggttc aggctcttcg ggcccagctg gaggcgtggc gtctccaagg ggaggctcct |
| 62221 | cagagtgcac tgagatccca ggaggatggc cacatccccc cgggctacat ctcacaggta |
| 62281 | aggccggggg gctgtttttc agggggaggg ggcagatttc tgcttgccgt gttatcagga |
| 62341 | aacagctgag attttgggag cccttcctgt gtgtgctgga acctgtgtgg ggcatttgac |
| 62401 | atgcgtcatc tcatttactc tccccacagc tggtgggcgt gatcactgtg cccgttttac |
| 62461 | agacaaggcc actgagctct gagaggttat gtgacttgcc caaggtcacc ccgcctgcag |
| 62521 | gtctcaaagg tgggatttga gcgagggtcc ggctgactgc agagcctgtg tgtgagtccc |
| 62581 | cgtgtgacac tctgcacttg gacccttgcc ccggggaagt ggggggctag tggccccgag |
| 62641 | agtcactccc tgaacacagg gaaatcctgc cgaggcccac tgggccaagg ccccacacaa |
| 62701 | ggaggtctgc aggacagggg agcctccagt agccctcaga gaatgcaccg tccgcctagg |
| 62761 | acagcggttc tcaacggggt ccattttgtc ccccttgggg acatttggct gtgtctggga |
| 62821 | atacttctgg ttgtcacaac taggacaaac taactctcta gaaaaaacgt gtgtgtgtac |
| 62881 | acatatataa gtttattata aattttacca ataaaaacaa tgcttagtac acaatttaca |
| 62941 | aataaaaata tataatttta ttgtgaattc cgtacagcaa atggattttt ctcagaatgc |
| 63001 | tttcagtgat tttttccata ctcttgtatc cacaactgat taacaaaggg ggttgttcta |
| 63061 | acgtgtgctg gttgacattt ttgtttatgg taatgagtaa gatgagaaga catttgtcga |
| 63121 | aactttactg gtccatgttg tcagctactt ctttgctaga tcggatagta gttttctatt |
| 63181 | gagaatattt gagaatattc tattaagaat atttttgcaa ttttttgttc tatttacaat |
| 63241 | gtaatggtta tagatgtgag atacttctaa gtttaatctg cattattagc attttcttat |
| 63301 | cacttgctta agtctagaca atcaacaaaa caataaatca agccctgatg tgtaatgttt |
| 63361 | accaattctg cagtgtaaat actcccgctg tggccaatgc tgcattacca gcacaaagtc |
| 63421 | cctggacaga gagttgggaa gagatgtcac actattcact aggatttcca ccatgcggat |
| 63481 | acagtagatg tgattggccc caagagcacg cataatacaa cacagtgaaa tatttaggaa |
| 63541 | gtaataaatt tgagtatcta ttgccttgtc tttaacatag ttaatttaaa gtatataaat |
| 63601 | ttaattgtta atggctgtgc ttaacaacta catcacagaa ttactaaaaa tttgacaatc |
| 63661 | agctgtctgt agttatcaca ccattatcta aagtagaaaa gaaggggcca ggcgcagtgg |
| 63721 | ctcacgcctg taatcccagc actttgggga ggccgaagtg ggcagatcac gaggtcagga |
| 63781 | gaacgagact aacctggcca acatggtgaa actccgtctc taataaaata caaaaaatag |
| 63841 | ccgggcgtgg tggtgcgcac tgtagtccca gctacttggg aggctgaggc aggggaccca |
| 63901 | ctggaaccca ggaggcgcag gttgcagtga gccgagattg cgccacggca ctccagcctg |
| 63961 | gcgacagagt aagactccat ctcaaaaaaa aaaaaaaaga aagaaaagaa aagaaggctg |
| 64021 | agatctaggc cctaggcagg aggagccttc taaaaatttc caaggggagg tcacgtcatg |
| 64081 | agggggctct caggaaggca ttggctgggg catgagaagg aaggctggtt ggtgcgtatt |
| 64141 | gtggatgcct ccagtgctga gctaatagtt agaagctctg gcctggtacg gtggctaatg |
| 64201 | cctgtatctc agcactttgg gaggtgaggt gggtggatca cttgaggtca aatgttcgag |
| 64261 | gccagtgtgg ccagagtggt gaaaccctgt ctctactaaa aaaaaaaaat acaaaactta |
| 64321 | gcagggcatg gtggtgggcg cctataatcc cagctactcc agtggctgag gcaggagaat |
| 64381 | cgcttgaacc cgggaggcag aggttgcagt gagctgagat cctgccactg cagcctgggt |
| 64441 | gacagtgaga ctctatctaa aaacagcaac aaaaaacaaa acaaacaaaa aaagagaagc |
| 64501 | tcattccaca gacttttgga ggtttggggt cagacagtga catcaggaaa gtgatgttgg |
| 64561 | agtggagggg gcttgggaga gggccaggtg taaggcatta ggaaatggct gaaggagggt |
| 64621 | gaatgtcaag ggtctggagg gaggagaatt tgggactgga tgaccagatc tacggaaggc |
| 64681 | agaggggcct ttgggggccc tgaggctggg gctgctgctc cagggaagcc cggagaccgt |
| 64741 | cctggggcca agtagtggat ggagtttcca ctgatagagg agcaggcagg ggaattgggg |
| 64801 | tgggacgcag agggtgaggc tgggggctta ccttgtcctg gggaggggca ggggaggagc |
| 64861 | cagtgttgtg ccctgtagcc tcccaccctg aggcagagaa gggagctggg ggcccaacac |
| 64921 | cccagctgag gcaggtggga gggcctccga ggcccccagc cagggggctt ctgcctgcgt |
| 64981 | gctctgctct aagaagctgc aggggctgct ttctgggtgg gtggggctgg gacccattcg |
| 65041 | tacccaccta taatcagagt ggcttcatct tcctccattt tattttattt taattttttt |
| 65101 | agagatgagg tcttttactg tcacccaggc tacagtgcag tggcacagag gctcactgca |
| 65161 | gcctcgaact ctagggctca agcagtcttc ccacctcagc ctcctgagta ggaaccacag |
| 65221 | gctacaccac catgcctggc taattaaaaa aatttttttg tagagacaag gtctcactat |
| 65281 | gttccctggg ctggtcttga actcctgggc tcaagtgctt ctctcatgtt ggcctcccaa |
| 65341 | agtgctgggg gtacaagtgt gaccactgca ctggcccacc tttgtccgtt ttactcatag |
| 65401 | agccagcttt agatttcctt tagataaagg ctctgccact acaatgacgt ttaaaaggca |
| 65461 | ctgctttggc ttctcagctt actggtggca aaactgaggc ccacagaagg gtgtgacctt |
| 65521 | tgagctatag gcagacctgt gtctggagtt caggcggtct ggtccccagg tgccccattg |
| 65581 | ggtgcaccaa agtctgtcag tgtttggtcc cgttagggtg ggatgggcac ggggagcagg |
| 65641 | tgggagggga gaagggaata actggggagc agagctgaag accaggcagg tggaggagtg |
| 65701 | accccagatg cggaaggcag cagagaagaa gagctgagtc atctgtatcg ccctcaggca |
| 65761 | cgggaggagg cggatggaaa ttcggtggac ctggaggagt gacagctggg agggattccc |
| 65821 | cagctggtat ctcgggctga gtgggtgccc ttccttcagg ggttgggggc ttgctgctgc |
| 65881 | acgacagctg tcgggtggtc tggggcttca cccactggac atctggtaaa gagaaccttt |
| 65941 | ccccactatt gcagtgggga cagaggcatt tctttatcta ctttgatgtc catgtcaaag |
| 66001 | gtggcagcca ggatcaggac accttgggcc actcaggaag gtaggtgaga gaggccctag |
| 66061 | gagcccctgt tcgtcagctg agtcccacct cctgatgtca gcccctgcac ccctgagctg |
| 66121 | ctgggtctgc ctgcctggtc tggccctctt cagaagagca gtgcccaccc ccacattgct |
| 66181 | gggctcccca ctggtggatg ctggcgtacc gtctccctct gtgcacaggg ttcccgtgcc |
| 66241 | cagggagcct cgggggctgg tcctgtgagg cctgctttac ggatgccaag actgaagctc |
| 66301 | agagggggcg aggggtttgt tcaggttcac tccaaggcgc agccagggtg aagaccgcct |
| 66361 | ctgaggtcca aagtcatgtt ctgttgtcct aagccaggag acacatagag ggcaggaaca |
| 66421 | gaagccaggg gacagacacc cctgtcgcca catgaagctc ttggccctcg ctcctccatc |
| 66481 | aaatcgtccc caggtgtggc tgctggagag gcggggaggt ggcctttgcg tgggggagac |
| 66541 | agtccctcat ctggtcctct gtgccttagt ttcttccctg gctcttcttc cccttccctt |
| 66601 | tttaatgacc ttaaacattt ttttaattat ataaaagaaa tgtttacatt ttcaaaaata |
| 66661 | atttaaacaa attgaaaggc tggaaatcaa caccctcaca ctccttctgc tgccagctgc |
| 66721 | caccccaggg tcctgtcggt ggctgctgcc gcctgcctat cagtttctgg tggcagctgt |
| 66781 | aacctgtaac cacagctacc taaataatgc acatgtatta cctcacgctt ctgtaggcga |
| 66841 | gaagtctaac gcgagtcttg ccgcgctgca gccaaggcgt gggcaggact gtgttcctct |
| 66901 | gcaggcaccc gttgtatccc ttgcctgctc cagcttctgg tggccgtccg cgtcccttgg |
| 66961 | ctctcggccc cttccttcat cttcaaagcc tgcagagggg aagtcgagtc cttctcctgc |
| 67021 | cacgtcactc tgaccatccc ttcctggtca cacctccctt gaactcttcg cttctgcccc |
| 67081 | cttcgtccac ttttaaggac ccttgtgatt acactgggct tgagggataa tctgggataa |
| 67141 | tctcccgatt ttaaggtcag ctgattagca accttgattc cctctgcagc ctgaattcct |
| 67201 | ctttgccatg taacatgatg tagtcacaga tcctgggagt aagatgtgga caactttgag |
| 67261 | gggctatcat tctgccccct acaatatcct ccctaccttt tctgtgtttt ctgtgttttc |
| 67321 | cattcactgg gacggacagg tctgcacgtt tgttcccgtg agtgagatca taccacgtgt |
| 67381 | atgtcagaag ccaactttca ccacctcatt ttgggatctt tttccacagc atccacacaa |
| 67441 | aaactctgtc tagcagctgc ctgtgtccca ttgtcaagct cgatcccatc atttattcaa |
| 67501 | gagttcccct gatagggatt tagattgttt ccagttttca tctgttacca acctggctgt |
| 67561 | agggaatacc cttgttccaa aagtctctgt acacatacga ctttctgtgg gatcatttct |
| 67621 | gggagtgaac ctactaggtc aaccagtggg gtcgcaaaat gtgatgatgc caccggacca |
| 67681 | cccccaacac acaccgttgt ggcggacact cccagcagtg ggtattatta gtctgagata |
| 67741 | cttgcccatc ttgtctacta agcattattt tgtattacat catcagtttc agctcattta |
| 67801 | ctgagcacct gccacatgtc aggccctggc ctagaccctg gggactgcgc cagtcagatg |
| 67861 | ttctcagtct tcagggaatt tatggtttgg cagaggtaga accttttgag gcaggggaga |
| 67921 | ggctgcccag agacagagct aggagagaca tttaaactgg gctgcaggtc agtaaattac |
| 67981 | ccagggaggt aaagttgcca aaaaaacatt ccaggccggg cacagtggct catgcctgta |
| 68041 | atcccagcat tttgggaggc caaggccggc agatcacaag gtcagaagtt caagaccatc |
| 68101 | ctggctaaca cagtgaaacc ccatctctac taaaaataca aaaattagct gggcgtggtg |
| 68161 | gtgggcacct gtagtcccag ctactcacga ggctgaggca gaagaatcac ttgaacccag |
| 68221 | aggtggaggt tgcagtgagc tgagattgct ccactgcact ccagcctggg tgacagaaca |
| 68281 | aaactctgtc tcaaaaacaa aaacaaaaac aaaaaaacca caaaattcca ggcagtggga |
| 68341 | atagctaaag ctcagaggtg tgaacgtgct gcaaccgtaa ctgtgagatt gcttggcaga |
| 68401 | gaaaggccag ggatgagaac gtaaaggtca gccatgggaa cggggacttg actctgtagg |
| 68461 | acgaggaatc cctggagggc ttgaggcagg agcgacaagg tgaggtttgt gaatgaggag |
| 68521 | ccgtggggtg ggggtgggag caggagcagg tgcagactgg aggtgctggg acccaggagg |
| 68581 | aggtggtgta cctgcccctg agccttctcc tctggaggct ggggaagggc cgctgtccag |
| 68641 | aggaacgggg cagcctgtga caccctggcg tccggcccac aggaggcatg tgagcgcagc |
| 68701 | ctggcagaga tggagtcctc gcaccagcag gtgatggagg agctgcagcg gcaccacgag |
| 68761 | cgggagctgc agcgcctgca gcaggagaag gagtggctcc tggctgagga gacggcagcc |
| 68821 | acggcctcag gtatggaccc tggggggggc acagtgggct gggctctgag cctctgtgct |
| 68881 | gaggttcctc ctagccaaac tggggaggat tttatcaaat aagatgtagg ggtctcagtg |
| 68941 | ccacatttgg ggtaaagggt cagcaggtgt tagaagctcc aagaactggc ctgtagccct |
| 69001 | acttaaaaag ggaaactgag gcagtaaaag ataccctccc cacccccaac accaccacta |
| 69061 | ccataggtgg tgactcttcc tgctcagggg cctgcagcca ccatagagtc tgcctaaagg |
| 69121 | cacatgctac ccacgtgcgt gggggagagg ctgatcagct gcatcccctg tcctcatagg |
| 69181 | agtgcctcct ccctgggcac atgggccaga ccctggggag agcaggggaa ggtgtgagca |
| 69241 | ttgtccatgc cagctgtgtg ccaggcactg ggccccacat gtttcccaca ggagcaccct |
| 69301 | tcatcctgga caattccctc ccctcctgcc accgcacacc tgctgtgtgc ctggcacatt |
| 69361 | cctaggcatc actaggggtt tgcagaacaa tagaaatgct ggccaatgtc acatgatctg |
| 69421 | aagctcacaa cctgccagtg ctgtgccaag tgctcatgca ctcgaaccca gccttggggc |
| 69481 | cagtgtgctc tgcatttcag gaagatcgcc tttgcagccg cgtgtgttag agaccccttc |
| 69541 | cccctcagca gagtctggac ttcgccctgt aatctggcac taaggtttct gctgtgaaat |
| 69601 | ggataaaaat cacacttaag tggaataata aagaccataa atagcctcat gtccgttcag |
| 69661 | ggtggggatt gccaccaaaa gatttaccag aggcccggtg cagtggctca tgcctgtaat |
| 69721 | cccagcactt tgggaggctg aggcgggcag atcacctgag gtcaggagtt cgagactagc |
| 69781 | ctggccaaca tggtgaaacc ccatctctac taaaaataca aaaattagct gggcgtgatg |
| 69841 | gtgggcagct gcaatcccag ctactcagga ggctaaggca ggagaatctc ttgaacccag |
| 69901 | gagacagagg ctgcagtgag ctgagattgc accactgcac tccagcctgg atgacagagt |
| 69961 | aagactccat caaaaaaaaa aaaggccagg cactgtggct catgcctgta atcccagcac |
| 70021 | tttgggaggc cgaggcaggt gcatcacgag gtcaggagtt caagaccagc ctggccaaga |
| 70081 | tggtgaaacc ctgtctctac tatgaataca aaaattagcc atgcgcggtg gcaggcgcgt |
| 70141 | gtaatcccag ctactcagga ggctgaggca ggagaatcgc ttgaaagtgg gcggcaaagg |
| 70201 | ttgtagtgaa ccaagattgt gccactgcac tccagcctgg gtgacagagt gagactccgt |
| 70261 | ctcctgggtg acagagtgag actccgtctc ctgggtgaca gagtgagact ctgtctcaaa |
| 70321 | aaaaaaaaga aagaaaaaaa ggagagaaac tttcaatgtt cagaagtttc ggattttaga |
| 70381 | attgtgggta aaaccatggg tggccctgag tgggaaaaaa aaagcctcgg tctcagtcct |
| 70441 | taggagctca caatctgaga ataaagccag caggagctaa tgttggggca tttactctga |
| 70501 | gccatgcttt gttctaaatt cttcacacac cttccaataa ccccatggag gaaaggtact |
| 70561 | gttgttatcg atccctgttt tccagataag ggatctgagg cacaaggggt taagtcacct |
| 70621 | gtgcaaagtc acatcggttt atgctgactg tggagcctga gctcttgacc acgaggctgg |
| 70681 | tcaaagcaga taggccttgt ttgagagccc tcagagtctg cagggggggt gtactaggtg |
| 70741 | gggtcctacc tggaaatcac tagaacagga ggtaccaatg gtcagaccct gagctgtctc |
| 70801 | cccagtaggg tccatccaac aggattatct tcccttaata gatgggctga gagaggttga |
| 70861 | gagcttaccc caagccactc agtttatgac acaacctggg ctactctaga ctcctggttc |
| 70921 | ggtgctcttt gcatagcact ccttggtcca agtccatagt acttgcatag tactatgtgg |
| 70981 | agtccacata gtactccacc agtacatgtg gcgtctcaga gctggtggca cttggtgctg |
| 71041 | ctgacccccc tccagtctct cttgtgcctc tctgccctgc ttccagccat tgaagccatg |
| 71101 | aagaaggcct accaggaaga gctgagccga gagctgagca aaacacggag tctccagcag |
| 71161 | ggcccggatg gcctccggaa gcagcaccag taagatgatg ccggggccca actgcccacc |
| 71221 | ctgatggtta tgggttgagg aactttgctg aggccccttg gcagcggaca catcagtttg |
| 71281 | cccctagctg agattcctgc tccagtctca tggatgcaag aaacagaagc agaaggtcct |
| 71341 | caggcagacc ccatgccagt cctcacagac agtccttcag gcagcttctt gtcttggctg |
| 71401 | gtcccagcct ccacccacct cttcacccct ttagtctccc ctggagctgg gctgcagtgt |
| 71461 | cactgttgac cactaggcct ctccctgccc cagagcacag ttttggaatg ttgaggtttc |
| 71521 | aggggcttta actttaaaga tgggcccctc cagccgggca cattggctca tgcctgtaat |
| 71581 | cccagcactt tgggaggctg ggatcaggag ttcgagacta gcctggccaa catggtgaaa |
| 71641 | ccccatctct actaaaaata caagctagct gaatgtggtg gcgggagcct gcaattctag |
| 71701 | ctacttggga ggctgaggca ggagaatcgc ttgaacccag gaggcagagg ttgcagtgag |
| 71761 | ccaagattgt gccattgtat tccagcctgg gcaacaagag tgagagcaag actccatcta |
| 71821 | aaaaaaaaaa aaaagatggg ctcctccaag ccaccctcac ttagggtaaa cacctgtagc |
| 71881 | ccagagaagc tgtcactggt tcacagcccc acagcaggct aaaggcaaac ctagagaggg |
| 71941 | aaccccagag tcctgggatg ctttaagtct acctgactgg actccaggct tggcaggagc |
| 72001 | aagacaacct atgctctcct ctgctcctcc tctggggcag gtcagatgtg gaggcactga |
| 72061 | agcgagagct gcaggtgcta tcggagcagt actcgcagaa gtgcctggag attggggcac |
| 72121 | tcatgcggca ggctgaggag cgcgagcaca cgctgcgccg ctgccagcag gagggccagg |
| 72181 | agctgctgcg ccacaaccag gtgggcctgg ccccaggttg gggggacacg ggtgggtccc |
| 72241 | ggcacccctc ccctgaccac cgtgcctctc ccaggagctg catggccgcc tgtcagagga |
| 72301 | gatagaccag ctgcgcggct tcattgcctc gcagggcatg ggcaatggct gcgggcgcag |
| 72361 | caacgagcgg agttcctgcg agctagaggt gagtgtcctc actagcacca ggcagcccag |
| 72421 | tggttctcgg ggcccggggc tatggggcac ccccgccata ggctgggtat cacctgtggc |
| 72481 | aggaaagcca agtctcccag tggctgggcc agcctgacta ggctagaaca cctactgtgc |
| 72541 | atccagtagc cagcattctg ccctcccacc tgcattgtgt cacctacctg ccgtgtacca |
| 72601 | tagcaaatca ctccactttt ctgagtccca gtagctcatc tgtaaaatgg caccagcacc |
| 72661 | ttccttactg ggaaatattg gggtaagaaa atatgtgtgt atgtgtgtga tgccagtatc |
| 72721 | actgtagtat tttctttctt tttctttctt tctttttttg agacagagtc tctctctgtc |
| 72781 | acccaggctg gagtgtagtg acacgatctc agttcactgc aacctctgcc tccctggttc |
| 72841 | aagcgattct cctgcctcag cctcccaagt agctaggatt acaggcctgc accaccacac |
| 72901 | ccagctaatt ttttatattt ttggtagaga caagtttcac tatgttggcc aggctggtct |
| 72961 | cgaactccgg acctcaagtg atctgcctac cttggcctca caaagtgctg ggattacagc |
| 73021 | atgagccacc acacccggcc ttagtattta tttctttact tttttttttt ttttgagaca |
| 73081 | gagttttgct gttgttgccc aggctacagt gcagtggcgc gatctcggct caccacaacc |
| 73141 | tccgcctccc aggttaaaac gattctcctg gccgggcgcg gtggctcacg cctgtaatcc |
| 73201 | cagcactttg ggaggccgag gcgggcagat cacgaggtag gagatcaaga ccatcctggc |
| 73261 | taacacagtg aaaccccgtc tctactaaaa atacaaaaaa attacccggg cgtggtggcg |
| 73321 | ggcgcggtgg cgggtgcctg tagtcccagc cactccggag gctgaggcag gagaatggca |
| 73381 | tgaacctggg aggcggagcg tgcagtgagc cgagatcgtg ccactgcact ccagcctggg |
| 73441 | tgacagagcg agactccgtc tcaaaaaaaa aaaaaatatt aaaaaaaaaa agcgattatc |
| 73501 | ctgcctcagc ctcccaagta gctaggatta caggcatgtg ccacctcgcc tggataattt |
| 73561 | tgtattttta gtagagacag ggtttttcca tgttggtcag gctcttctcg aactcccaac |
| 73621 | ctcaggtgat ccgcccactt cggcctctca aagtgttggg attacaggcg tgagccaccg |
| 73681 | tgcccagccg atattttatt tctctttttt tttttgagac ggagtcactc tgtcacccag |
| 73741 | gctggagtgc agtggcacca tctcggctca ctgcaagctc cgcctctcgg gttcatgcca |
| 73801 | ttctcctgcc tcagcctccc gagtagctgg gactacaggc acccgccacc acacctggct |
| 73861 | aattttttgt atttttagta gagacggggt ttcaccatgt tagccgggat ggtcccaatc |
| 73921 | tcctgacctc gtgatccgcc cgcctctgcc tcccaaagtg ctgggattac aggcgtgagc |
| 73981 | caccgctcct ggccccaata ttttatttct tcaacaaaca cttaggtgag gctcactctg |
| 74041 | tacctagaaa ttctggaagc cctttttgtc tgttaactca tctaatcttc ctgacagctc |
| 74101 | catacattag ctactatgac tgtccccatt tacagatgag gaaactgagg cacaggaagg |
| 74161 | tcaagtacct taccaaagct tttatagctg gactgggatt tgaaccaggc agtctggctc |
| 74221 | cagtctgagc ttttaagcct ccccagtcct ggctgaactt gtttcctcct tggtcaagtc |
| 74281 | agcagcacct agccctcggg gttggggagg ggttaatgag gtgccttgtg gagtggttca |
| 74341 | gcctgctcag gtttatgtag ggcttctttt catccatctc actcccgagg tgagagcctg |
| 74401 | gtgctgcatg agggctgcag cagacttgac cgtgagatat ccactcacac gggctggaca |
| 74461 | ggggtgagag taagggaagg caaagcaggg gaatcggaga gggcttcctg gaggagtccg |
| 74521 | cctccaggat gtagagggtt tttgtgcaga taggggctgc attctagggg cctgaggcag |
| 74581 | agagaacccg ggctgcaggg cactatgggc cagggtagct ctggcttctg gccctgggtc |
| 74641 | agtccagcac ctatatcccc aggtgctgct tcgcgtaaaa gaaaacgaac tccagtacct |
| 74701 | aaagaaggag gtgcagtgcc tccgggacga gctccagatg atgcagaagg taggtccttc |
| 74761 | cgctgggctg ggggccgtcg gggactctgg agccatctgg atgccatcct gtgagcacct |
| 74821 | gctctgtgcc aagccctcca ctcgcttcat ccttcctcag gctctcctgt gcttctccca |
| 74881 | tgtaggtgtc atcatcccca ttcactgaca gggaaactga gactaagaaa ggggaagtga |
| 74941 | cttccccaag gctgtacttg gcttctgttt atttagcatt atttaattta ttgattatta |
| 75001 | ttgagcacct actatatcct gggcactgtg ctaggtgctg ggaagacaga tgtgaacaaa |
| 75061 | ataaccccaa atctctgctg tggggagctt gcactccagt gagaataacc acgttgctag |
| 75121 | gagttgggtg ggtcagggtt cagcccaggt ctgagtctgc agcccgcctg gcccaccctt |
| 75181 | ggctgctatg tcactgcctg gtgcgctctg tcagtttggg gcaggaagtg gacatgttgg |
| 75241 | tggctctgta agtgcacgtg agccagaccg tgtttaccgc acttgaagaa caggtggcgt |
| 75301 | gaactcacag gaagggcccg agcacccagc caggctgacc tggttctcat cccagccact |
| 75361 | cactgccctg cggtcttggt caaaatcttc tcccccgaaa caggatgagg atgacaatga |
| 75421 | cgcctgtgtc cctgggtggt accagggagg tgggaggggt aagcccagaa cccacggcca |
| 75481 | tcttggggag ccacctggag ggatgaagcg aggtatctgc ggggaggtct ggctgctggt |
| 75541 | gctgcccatg tgcccggttg gcagggctgg agggagagac ttcatgccct catacctgcc |
| 75601 | tcctgccccc caggacaagc gcttcacctc gggaaagtac caggacgtct atgtggagct |
| 75661 | gagccacatc aagacacggt ctgagcggga gatcgagcag ctgaaggagc acctgcgtct |
| 75721 | tgccatggcc gccctccagg agaaggagtc gatgcgcaac agcctggctg agtagaggtg |
| 75781 | gatgccgagg cgtgtgccct gcagggtggg caggggctga ggctctccca cacccgggac |
| 75841 | tccctggacc accccagcca ggccacttcc ttcttctggc ctccaattcc cgttctgtaa |
| 75901 | aaaggacaat gaaaccagca acacggggtt gttgcagaga tcccatcaga cccgggccac |
| 75961 | aggctgcttc atcagcttaa gcctgttcct ccagagctgg gcggcacagt gcagagccac |
| 76021 | agcctactca gtgcaccccc cttcttgggg ctggaggcaa atgcagcgtg cacatccctg |
| 76081 | acccccacag tgaatctcca ctgagcttca cacacacaca tcgcacgctg aggccgggct |
| 76141 | tctatcccag ttagtcttaa cactccttct tgacttagca gggctcaaag tggttggttg |
| 76201 | gttggttggt tggttggttg gttggttggt tggttggttt tggttgcttt gagaaaggtc |
| 76261 | ttactctgac acccaggctg gagtgcagtg gtgcaatcac ggctcactgc agcctcaacc |
| 76321 | tcctaggctc aagggatcct cccacctcag ccttctgagt agctgggacc acaggccctc |
| 76381 | accaccatgc ccagataatt tttgcacttt ttgtataggt ggggtttcgc cgtgatttcc |
| 76441 | caggctggtc tcgaactcct gggctcaagc aatacacctg cctcagcctc ccaaaattct |
| 76501 | gagattacag gtgtgagccg ctgcacctgg ccaaagtgtt cttatttttg ctttttcaac |
| 76561 | gccacatcta cctggagcat cctctttctg ataagtcctc atggacttcc tatggcatgc |
| 76621 | aggagaggcc accctatggc tgagctgctt gggaaggagc caggaagagc ggatctggct |
| 76681 | gtaccttagg ggctgagagg tggtggaagg acccactcag cacccttgct tggccttggg |
| 76741 | gtggcctcgc agcccctcag cccaggcctc agccctgccg caccttgtct ttcaggtccc |
| 76801 | gcccagctgc agaccctcca ggctggagga ccagccgccc tccttccctc ctggatggaa |
| 76861 | gtaaaaagcc aagctttctc cccaccctct gtgggccaca cgtgcacttg cacccaccac |
| 76921 | acacacacac acacacacac acacacacac agacacacag acacatacgc acacacgtgc |
| 76981 | acacatgtac acacggatac acacacacac acacacactg catatctgag cgcgcccctc |
| 77041 | gcactgggtc tcaccttgca ccttcttcag gattttatat gtgaagagat ttttatatag |
| 77101 | atttttttcc tttttttcca aaacacttta tactttaaaa aaaaaaaaaa aaagcaattc |
| 77161 | ctggtggctg tgtgcctcca accctggtcc ccctctgtct ccagccaccc tctgcttggg |
| 77221 | cttctgagct ggtggccctg gcccagaggt ctggcggagg cccaggcagc agccatggcg |
| 77281 | gggtgtctct acaggggaga ggcgggagcc tgccaccctc ttcctgccct acctcctact |
| 77341 | aacacttcct gccccatttg gacccgtacc atggggctca ggacagaggg agctagcagc |
| 77401 | tggcctccat ggccccacag cctccttcga ggctgtgctg ggtgcagaac cgccagagcc |
| 77461 | acccaaaagg tgtttctctt ctgctccctg aacctcttaa cttaataaaa cgttccagca |
| 77521 | gctctggtgt cctagatggc tggcagaaca ggaatggaga ttggggtttc ttgaagtggc |
| 77581 | ttccccactc acaccctacc cacaccccac ccttcctaaa gcagcggcct ctaggcttct |
| 77641 | gagggtgggg ctgaaaatcc aaggtctccc ttagtacaga ctgtgacgcc cccagtgtgg |
| 77701 | cttgcaggct agtggcagcg gaagcatgtg ggaaatagca agtccagtcc caccccaacc |
| 77761 | tactgaacca gcgtctgcat tttaacaggg tgccccagtg attcacgtga ccttcaaagt |
| 77821 | ctgaggagcc ctggagtagg gcccagggcc caggcctgag agagaggcct ggggctaaga |
| 77881 | ctagccctga ccatgacctt gggcaagtca cacccctcac tgagcctcag tttgcttctc |
| 77941 | catcaaatgg gtgtttttct ttcagtccca gtcctctcct gaggcttttc ccagaggtca |
| 78001 | tgcccagccc tgggcagggg tgagggcagg gttgtggctg ccaggcacag gtccagcggg |
| 78061 | ctgggagtcc tcaccatggc tggctgtgct gattatggtt gcatggggct gggaagaggt |
| 78121 | cccatttctg agccacagaa acaggccaca tccagtaggg gtcctggaag gctgtgagaa |
| 78181 | cccagaggag accctgaccc agcctgcagg tagaagactg cctttggagg cagctttgaa |
| 78241 | agatgactct tccagatgga agaatcagga agggccttcc cacgtggagg caccagcagg |
| 78301 | agcaaacgtc cctggtgtgt tcaggcaaag agccacagga gagtgacctc gtggagtggg |
| 78361 | aagagattgt ttcccattcc cttgccaggg attggttgag gaatggacaa ggcttgcagt |
| 78421 | tcacgcaagc cagtgaaatg cgaggggaag tcttgggagt ggggtggagg aggtcatctg |
| 78481 | ggaagggtgt ccttggtctt aaaaagaggc acaagaggcc gggcgcggtg gctcttgcct |
| 78541 | attatcctag cactttggga ggccaaggca gggggatcac ttgaggtcag gagttcgaga |
| 78601 | ccagcctggc caacatggtg aaaccccgtc tctactaaaa atacaaaaaa ttagccagac |
| 78661 | gtggtgccaa gcacctgtaa tcccagctac tcaggaggct gaggcaggag aatcacttga |
| 78721 | acccaggagg cagaagttgc agtgagccaa gatcgcgcca ctgcactcca gcctgtgtga |
| 78781 | caagggcgaa actccatctc aaaaaaaaag ggggagggtg gcgcaaggag aggacacctc |
| 78841 | tctcctggcc ctgtcgccat cttccctcat caatagctaa aaatggcaaa gaagaaagag |
| 78901 | ccaggacccc gtgccatcaa atcctccggc cttgtgctgc cctgtctcgg cactgcttgc |
| 78961 | cttaggagat caggagtgtt cctagccact gggagatggg atgcctgcca ctggcagctc |
| 79021 | tgccgtccat cctcaccatc ccaccgtccc cccaagacag ctctccatcc catcctctgc |
| 79081 | ctcccttcta gcctcaacct aactcagggc tcccccgtct cacacctggg ttgcaggaac |
| 79141 | atcctcttca ccaacccccc tcccatccct cctcagggct gcctagggca gctacaaaaa |
| 79201 | ccaggccagc ctctgaaagg tgctccacat ctcattaaaa accgtgaaat cgcactgccc |
| 79261 | cactatcaga atagctcaga ggagaaggtc tgccagtacc aagtgctggt ggggatacgg |
| 79321 | agaagagcag ctcccaggcg ctgctggtgc aaatgtgctt tgttgcaccc acggcaggaa |
| 79381 | actgaccagc ttctcatcaa gttaaatacg catctaccct atggcctagc atttccactc |
| 79441 | ttgcttacac atccaggaga aatgagtcct tacatgcaca aaaggatgag taccgtaata |
| 79501 | ctcacatcag caaataaaca atacagctgt gtgtcagtag tagaatgaat aaattgtggt |
| 79561 | cgattcata |

### Stereocilin

The STRC gene encodes stereocilin, a protein that is normally expressed in the inner ear and is associated with the stereocilia of specialized hair cells in the inner ear (see, e.g., Verpy et al., Nature 456: 255-258, 2008; and Zhang et al., J. Med. Genet. 44: 233-240, 2007). Stereocilia, which are about 10-50 μm in length, are important for hearing and balance. Stereocilia play a mechanosensing role in hearing. By bending in response to sound, they form a structure for mechanoreception of sound stimulation.

The human STRC gene is located on chromosome 15q15. It contains 29 exons encompassing ˜19 kilobases (kb) (Verpy et al., Nature Genetics 29(3): 345-349, 2001; NCBI Accession No. NG011636.1). The gene is tandemly duplicated on chromosome 15q15 in a telomere-to-centromere orientation, with the tandem copies located less than 100 kb apart on the chromosome. The coding sequence of the second copy is interrupted by a stop codon in exon 20, meaning that it is a “pseudogene” that encodes a nonfunctional truncated protein. (Hereinafter, references to “the STRC gene” mean the gene that does not have that stop codon in exon 20, while references to the “pseudogene” or the “STRC pseudogene” denote the gene that does have that stop codon in exon 20.) In some examples, the full-length STRC protein is a full-length wildtype STRC protein. The full-length wildtype STRC protein expressed from the human STRC gene is 1775 residues in length, and contains a signal peptide and several hydrophobic segments. When the signal peptide is cleaved off during processing of the wildtype protein, the resulting mature wildtype stereocilin protein is 1719 residues in length. In some embodiments, a full-length STRC protein can include a heterologous signal peptide positioned N-terminal to an amino acid sequence of a mature wildtype stereocilin protein.

Various mutations in the STRC gene have been associated with hearing loss (e.g., non-symptomatic sensorineural hearing loss). For example, a homozygous 1-bp insertion was identified in exon 13 in a consanguineous Pakistani family with autosomal recessive non-syndromic sensorineural deafness-16 (DFNB16) (Verpy et al. (2001). Another example found in a family with DFNB16 includes both a 4-bp deletion in exon 5 and a larger deletion encompassing exons 17-29. The two deletions were inherited from the father and mother, respectively. The 4-bp deletion was predicted to result in the translation of 5 out-of-frame amino acids and a premature stop codon in exon 5.

Additional exemplary mutations in a stereocilin gene detected in subjects having non-symptomatic sensorineural hearing loss and methods of sequencing a nucleic acid encoding stereocilin are described in, e.g., Verpy et al., Nature 456:255-259, 2008; Verpy et al., Res. Systems Neurosci. 519:194-210, 2011; Hofrichter et al., Clinical Genetics 87:49-55, 2015; and Mandelker et al., J. Mol. Diagnostics 16:639-647, 2014. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype stereocilin protein is or includes the sequence of SEQ ID NO: 34. Non-limiting examples of nucleic acid encoding a wildtype stereocilin protein are or include SEQ ID NO: 35 and SEQ ID NO: 36. As can be appreciated in the art, at least some or all of the codons in SEQ ID NO: 35 can be codon-optimized to allow for optimal expression in a non-human mammal or in a human.

In some embodiments of any of the compositions described herein, the STRC protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 34.

Human Full-length Wildtype Stereocilin Protein
(Signal sequence shown in bold)
(SEQ ID NO: 34)
MALSLWPLLLLLLLLLLLSFAVTLAPTGPHSLDPGLSELKSLLSTLDQAPQGSLSRSRETTFLANISSSFE
PGRMGEGPVGEPPPLQPPALRLHDFLVTLRGSPDWEPMLGLLGDMLALLGQEQTPRDFLVHQAGVLGGLVE
VLLGALVPGGPPTPTRPPCTRDGPSDCVLAADWLPSLLLLLEGTRWQALVQVQPSVDPTNATGLDGREAAP
HFLQGLLGLLTPTGELGSKEALWGGLLRTVGAPLYAAFQEGLLRVTHSLQDEVFSILGQPEPDTNGQCQGV
FFLTLSLLGNLQQLLLWGVRHNLSWDVQALGFLSGSPPPPPALLHCLSTGVPLPRASQPSAHISPRQRRAI
TVEALCENHLGPAPPYSISNFSIHLLCQHTKPATPQPHPSTTAICQTAVWYAVSWAPGAQGWLQACHDQFP
DEFLDAICSNLSFSALSGSNRRLVKRLCAGLLPPPTSCPEGLPPVPLTPDIFWGCFLENETLWAERLCGEA
SLQAVPPSNQAWVQHVCQGPTPDVTASPPCHIGPCGERCPDGGSFLVMVCANDTMYEVLVPFWPWLAGQCR
ISRGGNDTCFLEGLLGPLLPSLPPLGPSPLCLTPGPFLLGMLSQLPRCQSSVPALAHPTRLHYLLRLLTFL
LGPGAGGAEAQGMLGRALLLSSLPDNCSFWDAFRPEGRRSVLRTIGEYLEQDEEQPTPSGFEPTVNPSSGI
SKMELLACFSPVLWDLLQREKSVWALQILVQAYLHMPPENLQQLVLSAEREAAQGFLTLMLQGKLQGKLQV
PPSEEQALGRLTALLLQRYPRLTSQLFIDLSPLIPFLAVSDLMRFPPSLLANDSVQQGYRGSEENSLEEDK
GLRPMTPRSLAAIRDYSPGMRPEQKEALAKRLLAPELFGEVPAWPQELLWAVLPLLPHLPLENFLQLSPHQ
IQALEDSWPAAGLGPGHARHVLRSLVNQSVQDGEEQVRRLGPLACFLSPEELQSLVPLSDPTGPVERGLLE
CAANGTLSPEGRVAYELLGVLRSSGGAVLSPRELRVWAPLFSQLGLRFLQELSEPQLRAMLPVLQGTSVTP
AQAVLLLGRLLPRHDLSLEELCSLHLLLPGLSPQTLQAIPRRVLVGACSCLAPELSRLSACQTAALLQTFR
VKDGVKNMGTTGAGPAVCIPGQQPIPTTWPDCLLPLLPLKLLQLDSLALLANRRRYWELPWSEQQAQFLWK
KMQVPTNLTLRNLQALGTLAGGMSCEFLQQINSMVDFLEVVHMIYQLPTRVRGSLRACIWAELQRRMAMPE
PEWTTVGPELNGLDSKLLLDLPIQLMDRLSNESIMLVVELVQRAPEQLLALTPLHQAALAERALQNLAPKE
TPVSGEVLETLGPLVGFLGTESTRQIPLQILLSHLSQLQGFCLGETFATELGWLLLQESVLGKPELWSQDE
VEQAGRLVFTLSTEATSLIPREALGPETLERLLEKQQSWEQSRVGQLCREPQLAAKKAALVAGVVRPAAED
LPEPVPNCADVRGTFPAAWSATQIAEMELSDFEDCLTLFAGDPGLGPEELRAAMGKAKQLWGPPRGFRPEQ
ILQLGRLLIGLGDRELQELILVDWGVLSTLGQIDGWSTTQLRIVVSSFLRQSGRHVSHLDFVHLTALGYTL
CGLRPEELQHISSWEFSQAALFLGTLHLQCSEEQLEVLAHLLVLPGGFGPISNWGPEIFTEIGTIAAGIPD
LALSALLRGQIQGVTPLATSVIPPPKFAVVFSPIQLSSLTSAQAVAVTPEQMAFLSPEQRRAVAWAQHEGK
ESPEQQGRSTAWGLQDWSRPSWSLVLTISFLGHLL
Human Wildtype Stereocilin cDNA
(SEQ ID NO: 35)
atggctctcagcctctggccoctgctgctgctgctgctgctgctgctgctgctgtcctttgcagtgactct
ggcccctactgggcctcattccctggaccctggtctctccttcctgaagtcattgctctccactctggacc
aggctccccagggctccctgagccgctcacggttctttacattcctggccaacatttcttcttcctttgag
cctgggagaatgggggaaggaccagtaggagagcccccacctctccagccgcctgctctgcggctccatga
ttttctagtgacactgagaggtagccccgactgggagccaatgctagggctgctaggggatatgctggcac
tgctgggacaggagcagactccccgagatttcctggtgcaccaggcaggggtgctgggtggacttgtggag
gtgctgctgggagccttagttcctgggggcccccctaccccaactcggcccccatgcacccgtgatgggcc
gtctgactgtgtcctggctgctgactggttgccttctctgctgctgttgttagagggcacacgctggcaag
ctctggtgcaggtgcagcccagtgtggaccccaccaatgccacaggcctcgatgggagggaggcagctcct
cactttttgcagggtctgttgggtttgcttaccccaacaggggagctaggctccaaggaggctctttgggg
cggtctgctacgcacagtgggggcccccctctatgctgcctttcaggaggggctgctccgtgtcactcact
ccctgcaggatgaggtcttctccattttggggcagccagagcctgataccaatgggcagtgccagggagtt
ttcttccttactctttccctcctaggtaaccttcaacagctgctcttatggggcgtccggcacaacctttc
ctgggatgtccaggcgctgggctttctgtctggatcaccacccccaccccctgccctccttcactgcctga
gcacgggcgtgcctctgcccagagcttctcagccgtcagcccacatcagcccacgccaacggcgagccatc
actgtggaggccctctgtgagaaccacttaggcccagcaccaccctacagcatttccaacttctccatcca
cttgctctgccagcacaccaagcctgccactccacagccccatcccagcaccactgccatctgccagacag
ctgtgtggtatgcagtgtcctgggcaccaggtgcccaaggctggctacaggcctgccacgaccagtttcct
gatgagtttttggatgcgatctgcagtaacctctccttttcagccctgtctggctccaaccgccgcctggt
gaagcggctctgtgctggcctgctcccaccccctaccagctgccctgaaggcctgccccctgttcccctca
ccccagacatcttttggggctgcttcttggagaatgagactctgtgggctgagcgactgtgtggggaggca
agtctacaggctgtgccccccagcaaccaggcttgggtccagcatgtgtgccagggccccaccccagatgt
cactgcctccccaccatgccacattggaccctgtggggaacgctgcccggatgggggcagcttcctggtga
tggtctgtgccaatgacaccatgtatgaggtcctggtgcccttctggccttggctagcaggccaatgcagg
ataagtcgtgggggcaatgacacttgcttcctagaagggctgctgggcccccttctgccctctctgccacc
actgggaccatccccactctgtctgacccctggccccttcctccttggcatgctatcccagttgccacgct
gtcagtcctctgtcccagctcttgctcaccccacacgcctacactatctcctccgcctgctgaccttcctc
ttgggtccaggggctgggggcgctgaggcccaggggatgctgggtcgggccctactgctctccagtctccc
agacaactgctccttctgggatgcctttcgcccagagggccggcgcagtgtgctacggacgattggggaat
acctggaacaagatgaggagcagccaaccccatcaggctttgaacccactgtcaaccccagctctggtata
agcaagatggagctgctggcctgctttagtcctgtgctgtgggatctgctccagagggaaaagagtgtttg
ggccctgcagattctagtgcaggcgtacctgcatatgcccccagaaaacctccagcagctggtgctttcag
cagagagggaggctgcacagggcttcctgacactcatgctgcaggggaagctgcaggggaagctgcaggta
ccaccatccgaggagcaggccctgggtcgcctgacagccctgctgctccagcggtacccacgcctcacctc
ccagctcttcattgacctgtcaccactcatccctttcttggctgtctctgacctgatgcgcttcccaccat
ccctgttagccaacgacagtgtacagcagggctacagagggtcagaggaaaacagtttggaggaagacaaa
gggttaagacccatgactcctcgcagcctggctgccatccgggattacagcccaggaatgaggcctgaaca
gaaggaggctctggcaaagcgactgctggccoctgaactgtttggggaagtgcctgcctggccccaggagc
tgctgtgggcagtgctgcccctgctcccccacctccctctggagaactttttgcagctcagccctcaccag
atccaggccctggaggatagctggccagcagcaggtctggggccagggcatgcccgccatgtgctgcgcag
cctggtaaaccagagtgtccaggatggtgaggagcaggtacgcaggcttgggcccctcgcctgtttcctga
gccctgaggagctgcagagcctagtgcccctgagtgatccaacggggccagtagaacgggggctgctggaa
tgtgcagccaatgggaccctcagcccagaaggacgggtggcatatgaacttctgggtgtgttgcgctcatc
tggaggagcggtgctgagcccccgggagctgcgggtctgggcccctctcttctctcagctgggcctccgct
tccttcaggagctgtcagagccccagcttagagccatgcttcctgtcctgcagggaactagtgttacacct
gctcaggctgtcctgctgcttggacggctccttcctaggcacgatctatccctggaggaactctgctcctt
gcaccttctgctaccaggcctcagcccccagacactccaggccatccctaggcgagtcctggtcggggctt
gttcctgcctggcccctgaactgtcacgcctctcagcctgccagaccgcagcactgctgcagacctttcgg
gttaaagatggtgttaaaaatatgggtacaacaggtgctggtccagctgtgtgtatccctggtcagcagcc
tattcccaccacctggccagactgcctgcttcccctgctcccattaaagctgctacaactggattccttgg
ctcttctggcaaatcgaagacgctactgggagctgccctggtctgagcagcaggcacagtttctctggaag
aagatgcaagtacccaccaaccttaccctcaggaatctgcaggctctgggcaccctggcaggaggcatgtc
ctgtgagtttctgcagcagatcaactccatggtagacttccttgaagtggtgcacatgatctatcagctgc
ccactagagttcgagggagcctgagggcctgtatctgggcagagctacagcggaggatggcaatgccagaa
ccagaatggacaactgtagggccagaactgaacgggctggatagcaagctactcctggacttaccgatcca
gttgatggacagactatccaatgaatccattatgttggtggtggagctggtgcaaagagctccagagcagc
tgctggcactgacccccctccaccaggcagccctggcagagagggcactacaaaacctggctccaaaggag
actccagtctcaggggaagtgctggagaccttaggccctttggttggattcctggggacagagagcacacg
acagatccccctacagatcctgctgtcccatctcagtcagctgcaaggcttctgcctaggagagacatttg
ccacagagctgggatggctgctattgcaggagtctgttcttgggaaaccagagttgtggagccaggatgaa
gtagagcaagctggacgcctagtattcactctgtctactgaggcaatttccttgatccccagggaggcctt
gggtccagagaccctggagcggcttctagaaaagcagcagagctgggagcagagcagagttggacagctgt
gtagggagccacagcttgctgccaagaaagcagccctggtagcaggggtggtgcgaccagctgctgaggat
cttccagaacctgtgccaaattgtgcagatgtacgagggacattcccagcagcctggtctgcaacccagat
tgcagagatggagctctcagactttgaggactgcctgacattatttgcaggagacccaggacttgggcctg
aggaactgcgggcagccatgggcaaagcaaaacagttgtggggtcccccccggggatttcgtcctgagcag
atcctgcagcttggtaggctcttaataggtctaggagatcgggaactacaggagctgatcctagtggactg
gggagtgctgagcaccctggggcagatagatggctggagcaccactcagctccgcattgtggtctccagtt
tcctacggcagagtggtcggcatgtgagccacctggacttcgttcatctgacagcgctgggttatactctc
tgtggactgcggccagaggagctccagcacatcagcagttgggagttcagccaagcagctctcttcctcgg
caccctgcatctccagtgctctgaggaacaactggaggttctggcccacctacttgtactgcctggtgggt
ttggcccaatcagtaactgggggcctgagatcttcactgaaattggcaccatagcagctgggatcccagac
ctggctctttcagcactgctgcggggacagatccagggcgttactcctcttgccatttctgtcatccctcc
tcctaaatttgctgtggtgtttagtcccatccaactatctagtctcaccagtgctcaggctgtggctgtca
ctcctgagcaaatggcctttctgagtcctgagcagcgacgagcagttgcatgggcccaacatgagggaaag
gagagcccagaacagcaaggtcgaagtacagcctggggcctccaggactggtcacgaccttcctggtccct
ggtattgactatcagcttccttggccacctgctatga
Human Wildtype Stereocilin cDNA - Codon Optimized
(SEQ ID NO: 36)
atggctctgtctctgtggcctctgctgctgctcctgttgctgcttctgctgctcagcttcgccgtgacact
ggctccaacaggaccccactctctggatcctggcctgagctttctgaagtccctgctgagcaccctggatc
aggctcctcagggcagcctgagcagatccagattcttcaccttcctggccaacatcagcagcagcttcgag
cctggcagaatgggagaaggacctgtgggagaacctcctccactgcaacctccagctctgcggctgcacga
ttttctggtcacactgagaggcagccccgactgggaacctatgctgggactgctgggagatatgctggccc
tgctcggacaagagcagacccctagagatttcctggtgcatcaggctggcgtgctcggaggactggttgaa
gttctgcttggagcactggtgcctggcggacctcctacacctacaagacctccatgcaccagagatggccc
cagcgattgtgtgctggctgctgattggctgcctagcctgctcctgcttctggaaggcacaagatggcagg
ccctggtgcaggttcagccttctgtggatcctaccaatgccaccggcctggatggaagagaagccgctcca
cactttctgcagggactgcttggactgctcacacctactggcgagctgggctctaaagaagccctttgggg
aggcctgctgagaacagttggagcccctctgtacgccgcctttcaagagggactcctgagagtgacacaca
gcctgcaggacgaggtgttcagcatcctgggacagccagagcctgacaccaatggacagtgtcagggcgtg
ttctttctgaccctgagcctgctgggcaacctgcagcaactgcttctgtggggcgtcagacacaacctgag
ctgggatgtgcaggcactgggctttctgtctggaagccctccacctccaccagcactgctgcattgtctgt
ctactggcgtgccactgcctagagcctctcagcctagcgctcacatcagccccagacagagaagggccatc
accgtggaagctctgtgcgagaatcacctgggacctgctcctccatacagcatcagcaacttctccatcca
tctgctgtgtcagcacaccaagcctgccacacctcagcctcatcctagcaccacagccatctgtcagaccg
ccgtttggtacgccgtttcttgggctcctggtgctcaaggatggctgcaggcctgtcacgatcagttcccc
gacgagttcctggacgccatctgcagcaatctgagcttctctgccctgtccggcagcaatcggagactggt
caaaagactgtgcgccggactgctgcctcctccaacatcttgtcctgagggactgcctcctgtgcctctga
ctcccgatatcttttggggctgcttcctggaaaacgagacactgtgggccgagagactgtgtggcgaagcc
tctctgcaagccgtgcctccatctaatcaggcctgggtgcagcacgtttgtcagggccctacacctgacgt
gacagcctctcctccttgtcacatcggaccttgcggcgagagatgtcctgatggcggcagctttctcgtga
tggtctgcgccaacgacactatgtacgaggtgctggtgcccttctggccttggctggctggccagtgtaga
atctccagaggcggcaacgatacctgtttcctggaaggactcctgggaccactgcttccatctctgcctcc
gcttggaccctctcctctgtgtcttacccctggacctttcctgctggggatgctgtctcagctgcctagat
gccagtctagcgtgccagctctggcccatccaaccagactgcactatctgttgcggctgctgaccttcctc
cttggacctggcgctggcggagctgaagctcaaggcatgcttggcagagccctgctgctgtcatccctgcc
tgacaactgcagcttctgggacgccttcagacctgaaggacgcagaagcgtgctgaggaccatcggcgagt
acctggaacaggatgaggaacagcctacaccaagcggcttcgaacccaccgtgaatcctagcagcggcatc
tccaagatggaactgctggcctgcttcagccccgtgctgtgggatctgctgcagagggaaaaaagcgtgtg
ggccctgcagatcctggtccaggcctatctgcacatgcctccagagaacctgcaacagctggtgctgtctg
ccgagagagaagctgcccagggattcctgactctgatgctgcagggaaagctgcagggcaaactccaggtg
ccacctagcgaagaacaggcccttggcagactgacagcactcctgctccagagataccccagactgacctc
tcagctgtttatcgatctgagccctctgatcccattcctggccgtgtccgacctgatgagattccctccta
gtctgctggccaacgactccgtgcagcagggatacagaggcagcgaggaaaacagcctggaagaggacaaa
ggcctgcggcctatgacacctagatctctggccgccatccgggattacagccctggaatgaggcccgagca
gaaagaggccctggctaagagactgctggctcccgagctgtttggcgaagttccagcctggcctcaagaac
tgctgtgggctgttctgcccctgctgccacatctgcctctggaaaacttcctgcagctgagcccacaccag
attcaggccctcgaggattcttggcctgccgctggactcggacctggacatgctagacacgtgctgagaag
cctggtcaaccagagcgttcaggacggcgaggaacaagtgcggagacttggacccctggcctgttttctga
gccccgaggaactgcagagtctggtgcctctgtctgaccctacaggccctgttgaaaggggcctgctggaa
tgtgccgccaatggaacactgagccctgagggcagagtggcctatgaacttctgggagtgctgagatctag
cggcggagccgttctgtcccctagggaacttagagtgtgggcacccctgtttagccagctgggcctgagat
tcctgcaagagctgtctgagccacagctgagggctatgctgcctgtgctccagggcacatctgtgacacca
gctcaagccgtcctgctgttgggcagactgctccctagacacgatctgtccctggaagaactgtgcagcct
gcacttgctgctgcctggactgtctcctcagacactgcaggccattcctcggagagtgcttgtgggagcct
gtagctgtctggcccctgagctgtctagactgagcgcctgtcaaacagccgctctcctgcagaccttcaga
gtgaaggatggcgtgaagaacatgggcaccacaggcgctggacctgccgtgtgtattcctggacagcagcc
cattcctaccacctggccagattgtctgctcccactgctgcccctgaaactgctgcagctggattctctgg
ctctgctggctaaccggcggagatattgggaactgccttggagcgaacagcaggcacagttcctgtggaag
aagatgcaggtccccaccaatctgacactgcggaatctgcaggctctcggcacacttgctggcggaatgag
ctgcgagttcctccagcagatcaacagcatggtggactttctggaagtggtgcacatgatctaccagctgc
caaccagagtgcggggaagcctgagagcttgtatttgggctgaactgcagcggcggatggccatgcctgaa
cctgaatggacaacagtgggccccgagctgaacggcctggactctaaacttctgctggatctccccatcca
gctgatggacagactgagcaacgagagcatcatgctggtggtggaactggtgcagagagccccagaacagc
tgctggcactgacacctctgcatcaagctgctctggccgaacgggcccttcagaatctggctcccaaagaa
acccctgtgtccggggaagtgctggaaacactgggacctcttgtgggcttcctgggcaccgagtctaccag
acagattcctctccagatcctgctgtcccacctgagccagctccagggattttgtctgggcgagacattcg
ccaccgaactcggatggttgctgctccaagagagcgtgctgggaaagcccgaactgtggtcacaggacgaa
gtggaacaggccggcagactggtgtttaccctgtctaccgaggccatcagtctgatccccagagaagcact
gggccctgaaacactcgagaggctgctggaaaagcagcagtcttgggaacagagcagagtgggccagctgt
gtagagaacctcagctggccgctaaaaaggccgcactggttgctggcgttgtcagacctgctgccgaggat
ctgccagagccagtgcctaattgtgccgatgtgcggggcacatttcctgccgcttggagcgctacacagat
cgccgaaatggaactgagcgacttcgaggactgtctgactctgtttgccggcgatcctggactgggaccag
aagaactgagagccgccatgggcaaagccaagcaactttggggacctccaagaggcttcagacccgaacag
attctgcagctcggccgcctgcttatcggactgggcgatagagagctgcaagaactgatcctggtggactg
gggcgtgctgtctactctgggacaaatcgacggctggtccaccacacagctgcggattgtggtgtccagct
tcctgaggcagtctggcagacatgtgtctcacctggacttcgtgcacctgactgccctgggctacacactg
tgtggactgcggcctgaggaacttcagcacatcagctcttgggagttcagccaggcagccctgtttctggg
aaccctgcatctgcagtgctccgaagaacaactggaagtgctcgcccatctgctcgtgctgccaggcggat
ttggccccatctctaattggggacccgagatcttcaccgagatcggcaccattgccgctggcatccctgat
ctggccctgtctgcactgctgagaggacagatccagggcgtgacaccactggccattagcgtgatccctcc
accaaagttcgccgtggtgtttagccccattcagctgtccagcctgacatctgcccaagccgtggctgtga
cccctgaacagatggcatttctgtctcccgagcagcggagagctgttgcttgggctcaacacgagggcaaa
gagagtcctgagcaacagggaagaagcaccgcttggggactgcaggattggtccagaccttcttggtcact
ggtgctgaccatcagctttctgggccacctcctgtga

A non-limiting example of a human wildtype stereocilin genomic DNA sequence is SEQ ID NO: 37. The exons in SEQ ID NO: 37 are: nucleotide positions 1-142 (exon 1), nucleotide positions 445-1230 (exon 2), nucleotide positions 1319-1343 (exon 3), nucleotide positions 2111-3368 (exon 4), nucleotide positions 4325-4387 (exon 5), nucleotide positions 4489-4605 (exon 6), nucleotide positions 4748-4914 (exon 7), nucleotide positions 5570-5756 (exon 8), nucleotide positions 5895-6010 (exon 9), nucleotide positions 6292-6424 (exon 10), nucleotide positions 6777-6959 (exon 11), nucleotide positions 7264-7302 (exon 12), nucleotide positions 7486-7653 (exon 13), nucleotide positions 7817-7882 (exon 14), nucleotide positions 8364-8489 (exon 15), nucleotide positions 9467-9525 (exon 16), nucleotide positions 10598-10721 (exon 17), nucleotide positions 10826-10938 (exon 18), nucleotide positions 13402-13537 (exon 19), nucleotide positions 13955-14151 (exon 20), nucleotide positions 14350-14440 (exon 21), nucleotide positions 14649-14805 (exon 22), nucleotide positions 15390-15559 (exon 23), nucleotide positions 17250-17405 (exon 24), nucleotide positions 17787-17929 (exon 25), nucleotide positions 18119-18267 (exon 26), nucleotide positions 18509-18605 (exon 27), nucleotide positions 18694-18841 (exon 28), and nucleotide positions 19041-19238 (exon 29). The introns are located between each of these exons in SEQ ID NO: 37, i.e., at nucleotide positions 143-444 (intron 1), nucleotide positions 1231 to 1318 (intron 2), nucleotide positions 1344-2110 (intron 3), nucleotide positions 3369-4324 (intron 4), nucleotide positions 4388-4488 (intron 5), nucleotide positions 4606-4747 (intron 6), nucleotide positions 4915-5569 (intron 7), nucleotide positions 5757-5894 (intron 8), nucleotide positions 6011-6291 (intron 9), nucleotide positions 6425-6776 (intron 10), nucleotide position 6960-7263 (intron 11), nucleotide positions 7303-7485 (intron 12), nucleotide positions 7654-7816 (intron 13), nucleotide positions 7883-8363 (intron 14), nucleotide positions 8490-9466 (intron 15), nucleotide positions 9526-10597 (intron 16), nucleotide positions 10722-10825 (intron 17), nucleotide positions 10939-13401 (intron 18), nucleotide positions 13538-13954 (intron 19), nucleotide positions 14152-14349 (intron 20), nucleotide positions 14441-14648 (intron 21), nucleotide positions 14806-15389 (intron 22), nucleotide positions 15560-17249 (intron 23), nucleotide positions 17406-17786 (intron 24), nucleotide positions 17930-18118 (intron 25), nucleotide positions 18268-18508 (intron 26), nucleotide positions 18606-18693 (intron 27), and nucleotide positions 18842-19040 (intron 28).

| Human Wildtype Stereocilin Gene (NCBI Accession No. NG_011636.1) | |
|---|---|
| (SEQ ID NO: 37) | |
| 1 | gccctgccct cacctggcta tcccacacag gtgagaataa ccagaactca cctccggtac |
| 61 | cagtgttcac ttggaaacat ggctctcagc ctctggcccc tgctgctgct gctgctgctg |
| 121 | ctgctgctgc tgtcctttgc aggtaagaag aacagtgagc agaactgggg atgaggagga |
| 181 | gggtggctgg aaaaagactt taagaatatg gaggtgaacc tgttagatag aaggacaaag |
| 241 | gagagaggca gagacttgtg caaaagggaa aaatgagggt taagaaaagc aggccaagac |
| 301 | ttactgtagg ccagtgaaag gggttcagct caccatcccc tcacctcatc tttagatcca |
| 361 | ggtagggaac tgtgctcagg ggcagggttg agtttgggct ctgtgttcct ctccttcagt |
| 421 | gacctctggt ttctctcctt acagtgactc tggcccctac tgggcctcat tccctggacc |
| 481 | ctggtctctc cttcctgaag tcattgctct ccactctgga ccaggctccc cagggctccc |
| 541 | tgagccgctc acggttcttt acattcctgg ccaacatttc ttcttccttt gagcctggga |
| 601 | gaatggggga aggaccagta ggagagcccc cacctctcca gccgcctgct ctgcggctcc |
| 661 | atgattttct agtgacactg agaggtagcc ccgactggga gccaatgcta gggctgctag |
| 721 | gggatatgct ggcactgctg ggacaggagc agactccccg agatttcctg gtgcaccagg |
| 781 | caggggtgct gggtggactt gtggaggtgc tgctgggagc cttagttcct gggggccccc |
| 841 | ctaccccaac tcggccccca tgcacccgtg atgggccgtc tgactgtgtc ctggctgctg |
| 901 | actggttgcc ttctctgctg ctgttgttag agggcacacg ctggcaagct ctggtgcagg |
| 961 | tgcagcccag tgtggacccc accaatgcca caggcctcga tgggagggag gcagctcctc |
| 1021 | actttttgca gggtctgttg ggtttgctta ccccaacagg ggagctaggc tccaaggagg |
| 1081 | ctctttgggg cggtctgcta cgcacagtgg gggcccccct ctatgctgcc tttcaggagg |
| 1141 | ggctgctccg tgtcactcac tccctgcagg atgaggtctt ctccattttg gggcagccag |
| 1201 | agcctgatac caatgggcag tgccagggag gtgagtgtgg ccagggctgg gactgggatg |
| 1261 | tggcagggca aggaaagtga aattggggta gttttcttcc ttactctttc cctcctaggt |
| 1321 | aaccttcaac agctgctctt atggtaagta acaggagacc agttctgagg gattgggcct |
| 1381 | ggaaaatctg gaggtgaaga gctgaagacc tcagcctcta gagaggaaaa ctgatgggag |
| 1441 | gagtgtagtt tagtggtttt ggggtgtgac tgtctgggtt ggtgtcccag ctccacctct |
| 1501 | tcctagccat atgaccttga gcaggttaca tagtctttct atacctcagt ttccccattt |
| 1561 | ataaaatgag aatgataata ttagttacca cagagttgtt gcacccggtt aaatgagttg |
| 1621 | atactgtgta tgcaaacgac ttaaaaccgt gctggcacat agcgcttaat aatgttagct |
| 1681 | agtaaagatg ggatttggaa aataaggaca cagctggatt cctctacccc cttactactt |
| 1741 | cagtacaaca atgccagaca gtagttagac atattgagtt gctgagcaga tttcctaaca |
| 1801 | tgaggcccgc tgagggttgt gtttaagcta tctaaaagca tacgaagaaa ggagacagaa |
| 1861 | gggggccagg tggacagaaa gaattccaac tggggcttct cctaggtgat tttggacctt |
| 1921 | ggcagggcag ctttctcttt tttgccccgt tgcagcattt caaccagtaa cgcctaaact |
| 1981 | ctcagggacc tcgcttgtag aaaagcctat gcttgccatg ccccttgagg gctctgagtc |
| 2041 | agggtcagaa tcttcagctg gaggaaatgt gaactgacca gatcctgcct gctcctccct |
| 2101 | ctgcacccag gggcgtccgg cacaaccttt cctgggatgt ccaggcgctg ggctttctgt |
| 2161 | ctggatcacc acccccaccc cctgccctcc ttcactgcct gagcacgggc gtgcctctgc |
| 2221 | ccagagcttc tcagccgtca gcccacatca gcccacgcca acggcgagcc atcactgtgg |
| 2281 | aggccctctg tgagaaccac ttaggcccag caccacccta cagcatttcc aacttctcca |
| 2341 | tccacttgct ctgccagcac accaagcctg ccactccaca gccccatccc agcaccactg |
| 2401 | ccatctgcca gacagctgtg tggtatgcag tgtcctgggc accaggtgcc caaggctggc |
| 2461 | tacaggcctg ccacgaccag tttcctgatg agtttttgga tgcgatctgc agtaacctct |
| 2521 | ccttttcagc cctgtctggc tccaaccgcc gcctggtgaa gcggctctgt gctggcctgc |
| 2581 | tcccaccccc taccagctgc cctgaaggcc tgccccctgt tcccctcacc ccagacatct |
| 2641 | tttggggctg cttcttggag aatgagactc tgtgggctga gcgactgtgt ggggaggcaa |
| 2701 | gtctacaggc tgtgcccccc agcaaccagg cttgggtcca gcatgtgtgc cagggcccca |
| 2761 | ccccagatgt cactgcctcc ccaccatgcc acattggacc ctgtggggaa cgctgcccgg |
| 2821 | atgggggcag cttcctggtg atggtctgtg ccaatgacac catgtatgag gtcctggtgc |
| 2881 | ccttctggcc ttggctagca ggccaatgca ggataagtcg tgggggcaat gacacttgct |
| 2941 | tcctagaagg gctgctgggc ccccttctgc cctctctgcc accactggga ccatccccac |
| 3001 | tctgtctgac ccctggcccc ttcctccttg gcatgctatc ccagttgcca cgctgtcagt |
| 3061 | cctctgtccc agctcttgct caccccacac gcctacacta tctcctccgc ctgctgacct |
| 3121 | tcctcttggg tccaggggct gggggcgctg aggcccaggg gatgcbgggt cgggccctac |
| 3181 | tgctctccag tctcccagac aactgctcct tctgggatgc ctttcgccca gagggccggc |
| 3241 | gcagtgtgct acggacgatt ggggaatacc tggaacaaga tgaggagcag ccaaccccat |
| 3301 | caggctttga acccactgtc aaccccagct ctggtataag caagatggag ctgctggcct |
| 3361 | gctttagtgt gagtgctctg ccagagggaa agctcctaga acagtgagaa ggccctccag |
| 3421 | gggaattcct cgaatactca gaggcagtag tgtggggtag tagttgaagc acacagctct |
| 3481 | agagtcagac aggcttggat tcatatcttg gttctgtgac cagccttgaa tgagttattt |
| 3541 | aacttctctg agcaatattt ttctcgtctc atttataaac tagggatgat aatggtatat |
| 3601 | gagataatac atgcbgtggg cttagcacag tgcatgatac acaaacatgc aataaatatt |
| 3661 | accttgttat tcttttgggc tctttgactc tctcactttc tgcaccagaa agaaaaagga |
| 3721 | tcaagttaga ggactctaaa tttttcccct agagagtgag aattggaggc tggcagaata |
| 3781 | caggaagata aggtaggaat gagaaagatt cagggacact accaatcaga agactttggt |
| 3841 | tctaggttca actgtgccac aaattagtgt gatcttaggc aagcaatttc atttagtttt |
| 3901 | tctgggcttc agtttttagt ctgtagaatg gaggggtgag aatatgttaa acaccataat |
| 3961 | taattcactg agtgcctatt atatgcaagg cactttgcta ggttctgtag gatatataaa |
| 4021 | gatttcttac tccatgttgg ggccaccttt ttcaaaccct gggcccagta aaatggaatt |
| 4081 | agatagtctc atagtatttg gttcaggtct acaagtatta attgagccaa ctatggacct |
| 4141 | ggcatgggag agggtacaag agaaattaga gatatgatcc cggacctaaa agagcttaat |
| 4201 | atctgaagaa tcacacttga gatgatggac aagcatccca gcaagtggag ctggaatgcc |
| 4261 | tgggggagct gcaggagaga cagagaagac agctctgttg gcatattgtc tttcttccca |
| 4321 | ccagcctgtg ctgtgggatc tgctccagag ggaaaagagt gtttgggccc tgcagattct |
| 4381 | agtgcaggta acaggtggag ggcacatggg tgggcbgggt gacagccatg gctggaggtc |
| 4441 | cctgccccgt gaggtgaggc catacccacc atgacctcct attcgcaggc gtacctgcat |
| 4501 | atgcccccag aaaacctcca gcagcbggtg ctttcagcag agagggaggc tgcacagggc |
| 4561 | ttcctgacac tcatgctgca ggggaagctg caggggaagc tgcaggtgag cactgagaaa |
| 4621 | ggggagcaag ggcacctgga gcctagtgtt cagagggctt gctttagtgg gaggaggaac |
| 4681 | tccagagagg aaatggcagg gatactgagc atctccagag gcagaatcca ttcctgtgcc |
| 4741 | cctacaggta ccaccatccg aggagcaggc cctgggtcgc ctgacagccc tgctgctcca |
| 4801 | gcggtaccca cgcctcacct cccagctctt cattgacctg tcaccactca tccctttctt |
| 4861 | ggctgtctct gacctgatgc gcttcccacc atccctgtta gccaacgaca gtgtgtaagg |
| 4921 | ttcttgcact actcctcctg ctcctgtcac ggtcaggcca accgcatcca cctggagcag |
| 4981 | ccccttccgg agctcctctc tgtttttttc tttcatgcca gataggcaat gtgccaacat |
| 5041 | cgtagcaagg tttgagagag gcacatctca cgcctgagtg tgaaaaccca atcattatgc |
| 5101 | taatgaacta caaaaggatc agagagctcc tctctattaa aaccagggag aggatgggcg |
| 5161 | tggtggctca tgcctgtaat cccagcacgt tgggagcccg aggcaggtgg atcactaggt |
| 5221 | ccgcctagtg agttcgagac cagcctggcc aatatggtga aaccccgtct ctattaaact |
| 5281 | acaaaaatta gccaggcatg gtggtgggcg cttgtagtcc cagttactct ggaggctgag |
| 5341 | gcaggaggat agcttgaacc tgggaggcag aggttgcagt gaaccaagat cgtgccactg |
| 5401 | cactccagcc tgggtgacag agcgagactc cgtcttaaaa aaaacaaaaa acaaaacaaa |
| 5461 | acaaaaaaac agggagagtc tccttcctat ctagacagca gggctacaga gggtcagagg |
| 5521 | aaaacagttt ggaggaagac aaagggttaa gacccatgac tcctcgcagc ctggctgcca |
| 5581 | tccgggatta cagcccagga atgaggcctg aacagaagga ggctctggca aagcgactgc |
| 5641 | tggcccctga actgtttggg gaagtgcctg cctggcccca ggagctgctg tgggcagtgc |
| 5701 | tgcccctgct cccccacctc cctctggaga actttttgca gctcagccct caccaggtat |
| 5761 | gagaatcatc ttctttactt gactggccca tcttctgcta gtggggacaa agagtcaatg |
| 5821 | gcatgtctct cagtggcccc tccctgcaag aaccctatag tgaccccagt gcgagctaac |
| 5881 | cttccccatc tcagatccag gccctggagg atagctggcc agcagcaggt ctggggccag |
| 5941 | ggcatgcccg ccatgtgctg cgcagcctgg taaaccagag tgtccaggat ggtgaggagc |
| 6001 | aggtacgcag gtgagttgtt gtgggatcag taaccaaggc aagagtggaa gaggtagaga |
| 6061 | gaggaaggca cagcbgtcac gcbgggtcgg tgttctagga agaaaggggc aagagagtag |
| 6121 | gcagtggcct caggcagcat agagttccag gagagaggtc tatagatggt gcccctgtgt |
| 6181 | agtggtgtag tgtcagagtg cccagtgtat gtacccatac catctgctgc caggcctgcc |
| 6241 | ttagtgctag tcttggggac cacacaaagg tcagcttcat gccctcctca ggcttgggcc |
| 6301 | cctcgcctgt ttcctgagcc ctgaggagct gcagagccta gtgcccctga gtgatccaac |
| 6361 | ggggccagta gaacgggggc tgctggaatg tgcagccaat gggaccctca gcccagaagg |
| 6421 | acgggtgagc ccctcagcac aagcctacaa gactttaggc ttcccctggg tctgtgtgga |
| 6481 | tggctttccc attgtgtcaa cttgagcaca gtggtgccag cccccatccc acttttgcaa |
| 6541 | cctccattcc ttactccatg gccattctta cctgttacca cctcttcctg gcccttctct |
| 6601 | atctggtctg tagcacccca aacataccct ttgccatttt gaacctaatc tactccagtc |
| 6661 | caatccctag ttccaaaccc tagcccaggc cctgggaaat tcagatgtgg gattagagag |
| 6721 | gaagttcaag gttcatctgt cttttctctc cagtcctaaa ccttctttgg ttacaggtgg |
| 6781 | catatgaact tctgggtgtg ttgcgctcat ctggaggagc ggtgctgagc ccccgggagc |
| 6841 | tgcgggtctg ggcccctctc ttctctcagc tgggcctccg cttccttcag gagctgtcag |
| 6901 | agccccagct tagagccatg cttcctgtcc tgcagggaac tagtgttaca cctgctcagg |
| 6961 | tttgcctgtc tcactccctg gcatgtaccc tccatccccg cttgagcccc agtcaagaga |
| 7021 | atcccattca gggataaaag cagcccctcc tttccctggg tgaacagtag aggtaaactc |
| 7081 | tgtctgcagg aggacgcctt cattcccttt cctcagatca agaagggacc tgagtcactg |
| 7141 | aggatggtta ctagggatgg ttaagaggca gcgggaagtt ttggagggtt tgccttagga |
| 7201 | acccacttag gacctggctg ctgggtcctg agagcbgttg ttttcggtcc catcccaaca |
| 7261 | caggcbgtcc tgctgcttgg acggctcctt cctaggcacg atgtgagtag cagcaacttc |
| 7321 | tcagcctccc gccagaggtc tctatcctct tttaacctgg ctcctgcatc tgcccctcct |
| 7381 | ctctctccgc tcccctcata cttactgcct tgctgcattg tgattgttgt cttccccaac |
| 7441 | acccttccct tcttcttcag gcctcttgtc tctcttgctc tttagctatc cctggaggaa |
| 7501 | ctctgctcct tgcaccttct gctaccaggc ctcagccccc agacactcca ggccatccct |
| 7561 | aggcgagtcc tggtcggggc ttgttcctgc ctggcccctg aactgtcacg cctctcagcc |
| 7621 | tgccagaccg cagcactgct gcagaccttt cgggtatgag agtggcaagg aggatgagat |
| 7681 | aatcagggat accggctctt tctggttggg aggaaggcat cttccctgag gccagggaag |
| 7741 | gcctttcata cctccccact tacacacaca cacacacaca cacacacaca cacacacaca |
| 7801 | accaattctc atgcaggtta aagatggtgt taaaaatatg ggtacaacag gtgctggtcc |
| 7861 | agcbgtgtgt atccctggtc aggtaagtgt gagatctccc aactgagctc ctctccccat |
| 7921 | tctggggcag tttcatatgg ctggtgctac ctcccacact accctgcagt ggccctgaga |
| 7981 | gttctggtta gctctgtgcc cattagcagc cctccccagt gccagatgca ggacagcatg |
| 8041 | atccactcac attgtcctag actaatgtca aagctggaag ggcctgagaa atcttccagg |
| 8101 | ccacccaccc tgctttcaga tgaaaagacc aaggcbggga gaagctaagg gactttgttt |
| 8161 | gcctggtgcc taactagcag caacacttga ccacagcagc ctgcagtgtg aggctcttag |
| 8221 | gcgtttattg ctacagtggc aaatgccatt ccacttctgt cctagctttg gtccctttcc |
| 8281 | acccccatgg ttccttttct ctgagtgcta agtacagact ctctcaccta tcactacact |
| 8341 | gctataccca tcaccgccag cagcctattc ccaccacctg gccagactgc ctgcttcccc |
| 8401 | tgctcccatt aaagctgcta caactggatt ccttggctct tctggcaaat cgaagacgct |
| 8461 | actgggagct gccctggtct gagcagcagg taattctccc cacttaattt cagaacttcc |
| 8521 | tccctcaatg tagtctacct tctttaccta tcccttagcc ctatttggcc agcttatccc |
| 8581 | tactatcctt tatttgattg tttgagatac agtctcactc tgttgcccag gctgcagtgc |
| 8641 | agtggcatga tcagagttcg ctgtaacctc aaactcctga gctcaggcaa tctttctgcc |
| 8701 | tcagcctcct gaatagctag gacgacaggt ggttaccacc atgcctggct aatttttaaa |
| 8761 | tttttttttt gttttttgag atgaagtctt gctctgtcac ccaggcttga gtacagtggc |
| 8821 | acaagcttgg ctcactgcaa cctctgtctc ccgggttcaa gcgattctcc tgcctcagcc |
| 8881 | tcccgagtag ctgggactac aggcactccc cacaatgcct ggctaatttt ttttttgttt |
| 8941 | tagtagagac agggtttcac catattggcc aggctggtct cgaactgctg accttgtgat |
| 9001 | ctgcctgcct ctgcctctca aagtgctggg attacaggtg tgagccacca tgcccggcca |
| 9061 | atttttaaat tttttgtaga gacagacaat acaaaaatgt ggacactatg tggagacact |
| 9121 | atgttgaggt actatgctgt ccagattggt cttgaactcc tggcctcaag caatcctcct |
| 9181 | gccttggcct cccaaagtgc tgggattaca gacctgagcc actgcaccca gccccctagt |
| 9241 | atctcttata atgtgacttg cttttctttt tctttctcct tcccttttct ttcatttctt |
| 9301 | tctcactctc gagagaagag tgggcatctg ggagagtggg aggctggtgg gtcccacaga |
| 9361 | gtgaggaggc aggactgggt ccaaggcagt cctgcctctc cactctaggg ggtatccttg |
| 9421 | gacagtgtct cttctgggaa ggggctcgtc tttctttctc ttgtaggcac agtttctctg |
| 9481 | gaagaagatg caagtaccca ccaaccttac cctcaggaat ctgcagtgag taacttgtgt |
| 9541 | tgagcagtgc gctgaattcg accaacattt ttttgagtgc ttactatgtg ccaggcacca |
| 9601 | tgtgatatgg aatgggggat atagggatga atgatgcata gtccctgcct cgtggacgtt |
| 9661 | ctcctagcac ctccctttgc cctcctttcc ttccacagtg ccatgcctat cctgactaga |
| 9721 | gccaaaggac tcagaaaacc tggattcagg ttccagtcct gtcacctact tgtcctcttg |
| 9781 | ggcaagtcat ttaacgtccc tgtgtcagtt ttcccttctt taaatgagaa ttacaatggc |
| 9841 | accagcctca taggtagtta ctgtgaagat taaatgaggt aggtcatgta agatatttaa |
| 9901 | cacagtgttt ggtccattgt aaagtcccag tagtcatttg ctactgttag tttacttcag |
| 9961 | gatgacttca gaggcactgg ccaagcaaga ataaatagga ataagaaggt atcactttac |
| 10021 | ttacacccac attagaagaa caatgggctt cagaatcttt tttttttttt ttttttcgag |
| 10081 | acagtcttgc tctgttgccc aggctggagt gcagtggcgc gatttcggct cactgcaacc |
| 10141 | tctgcctccc aggttcaagc gattctcctg tctcagcctc tggagtagct gggattacag |
| 10201 | gaatgtgcca ccatacccag ctaatttttg tatttttagt agagatgggg tttcaccatt |
| 10261 | ttggccaggc tggtctcaaa ctcctgacct caggtgatcc acccgcctca gcctcccaag |
| 10321 | ggcttcagaa tctaagacat ggctctagtt tcagtttacc acatttctag cagaatgatg |
| 10381 | ttgggaatgt cacctgactt ccataaatcc ttattttctc ctctgataaa cagcagtgat |
| 10441 | gttatgggga gctgatgaga tatctatgta aaaacatttc tcaaaccata aattacggtg |
| 10501 | gatgaacatc tgtacttgtg ttgagagtac tgatatcaag gagcaaacag gctgttgtat |
| 10561 | gtgttgaatg agcctctccc cactcacaca cccacagggc tctgggcacc ctggcaggag |
| 10621 | gcatgtcctg tgagtttctg cagcagatca actccatggt agacttcctt gaagtggtgc |
| 10681 | acatgatcta tcagctgccc actagagttc gagggagcct ggtgagaggg ggtgcctgga |
| 10741 | ctttagtggg agcagggagg ctgggaccct aggtatagaa cccagctcct atgttctgct |
| 10801 | ctggcctcac actgcttccc tacagagggc ctgtatctgg gcagagctac agcggaggat |
| 10861 | ggcaatgcca gaaccagaat ggacaactgt agggccagaa ctgaacgggc tggatagcaa |
| 10921 | gctactcctg gacttaccgt aagtactgca gctagagata ttggcccctc agaaagctca |
| 10981 | atctggggtg aagatctgcc cttagggaat gccctggagg aggtagtttt tctgtctggt |
| 11041 | agttccctga cataatttat agcccaaagc agaggatttt attcaaagtt gctctatgta |
| 11101 | ttgactggtt cccagaatat gctccagcac agggcagctg agggtggcaa cactgtattg |
| 11161 | aagcctgcca agtaatctta caataaccta gtccacatta attgagattg agacagagca |
| 11221 | tctgaagtga gggaggcaat gctccaaatc tgccccagag gattgtagtt tgctcagggc |
| 11281 | actgtgttct tagtgcattc agaggagtag atcgagagaa aaatatatga aaaatgtgat |
| 11341 | aaataccttc aaatacctga ggggctatca agtagaaatt agattgtcat atttatgagt |
| 11401 | ggccccattg ggcaagacta agagtagtta acggagatca gatttttaca tagtataaga |
| 11461 | aaaactaagg tagtgagttc ctggtccttg gagcbgttcg agcctaagcc agatggcccc |
| 11521 | atggcaggaa tgttgtagag cacgttcata tacaggttgt gggaagaaaa ggctatagga |
| 11581 | acccaaggct cctccctacc catggagaaa tttattagta tgttactcat atgctgcttt |
| 11641 | tctcatttta cccctaccac caccccgttg ccatccgcac tgtaagtcag gataggaaaa |
| 11701 | tgcbggtgtt acagtcttcc tggggaatat ggagctgaag tggagtaaaa gcagttgact |
| 11761 | tcattcctac ttttttcttt tttttctttt tttttttttt tgagacagag ttttgctgtg |
| 11821 | tcaccaaggc tggagtgcag tgacgtgatc tcggctcact gcaacctcca tcttccaggt |
| 11881 | tcaagcaatt ctcctgcctc agtctcccga gtagcbggga ctgtaggtgt gcaccaccat |
| 11941 | gccaggctaa tttttgtatt tgttgtaggg acgagctttc accatgttgg ccaggctggt |
| 12001 | cttgaactcc tggcttcaag tgatctgccc acctcggctt cccaacattc ttatattttt |
| 12061 | ataggccttt ccacagattt cagctcttgt atgacttagc ccagttccag aactggtaat |
| 12121 | cctaggtagg gtacaggtta tcacctctga tttcgggtaa aagggattta tttatttatt |
| 12181 | tgtttattta tttatatttt tgagacagag tctcgctctg tcacccaggc tggagtgcaa |
| 12241 | tggtgccatc tcggctcact gcaacctctc cctctggggt tcaagcaatt ctcctgcctc |
| 12301 | agcctgctga gtagcbggga ttacaggcgc gtgccaccac acccggctaa tttttgcatt |
| 12361 | tttagtagag acggggtttc accatgttgc tcagggtggt ctcgaatttc tgaccctgtg |
| 12421 | atctgcctgc ctcggcctcc caaagtgctg ggattacagg catgagccac tgcgtccggc |
| 12481 | ctgtttttac ttttttttaa tgccattcag atctgtttaa atatgtgggt tctgtgagat |
| 12541 | aatttagaat cccaaggtta cagatgaggt gaaagatcct agaccatgca tcaaaaaact |
| 12601 | tgagtttctc atttgtgaaa gaaggataag agaaacacct attttgtctg ggtgcagtgg |
| 12661 | ctcatgccta taatcccagc atttggggag gccaaggtgg gtggatcacg gaggtcaggt |
| 12721 | gttcaagacc agactggcca acatggcaaa acaccatctc tactaaaaat acaaaagtta |
| 12781 | gcbgggcgtg gtggcacgtg cgtgtaattc cagctattcg ggaggctgag gcacgagaat |
| 12841 | tgcttgaacc tgggaggtgc gggttgcagt gaactgagat cgcagcacca ctgtgctcca |
| 12901 | gcctgagtga tggagtgagg ccaggtcttg ttgtaggatc aaatgagata acacctgaaa |
| 12961 | gaactttgta aattgtatag cacgtacaaa caagaaggga cctcttcaca agcagaggaa |
| 13021 | gggtggtcct gtggaaaaaa acgggaattg ggagtgagag acctcaacat ttgatctctg |
| 13081 | tgaacctcag ttttttaatc tataaaatgg ggaaatgtta atggtactta atatttggag |
| 13141 | cttttgagtc cattagatca ggtaggattg ttcgttattt ttttttttta ggaagactag |
| 13201 | aaatatgttg ctcccttttt ctcccccact caagcttgat ggtgggaatt ggccctggag |
| 13261 | ctgtttacta tcagttcctg tccagcttca ctaaatttgg tctggggtca catcttagct |
| 13321 | gcggactgtg gggttttgtg gtcccttctc gacttggccc agctccacct gaatcctgtt |
| 13381 | gttgtcaaat tgctgtaata ggatccagtt gatggacaga ctatccaatg aatccattat |
| 13441 | gttggtggtg gagctggtgc aaagagctcc agagcagctg ctggcactga cccccctcca |
| 13501 | ccaggcagcc ctggcagaga gggcactaca aaacctggta agagtccacc ctaccagact |
| 13561 | cagatttgct gccctgggca attcttgctc ctcagacaat gctctctgac tgtcccccaa |
| 13621 | ccctctactt cttgctttct tgctgccaaa cagattcctg tctacaaggc ctggcccctg |
| 13681 | ttttgcctct gggttctgtt ccttgataat atgcttcacg ttacttgtcc atacctcttg |
| 13741 | gagtccgaga aatctcttgg agtccacctc tcagtctttc tgcctgctcc tatctgggct |
| 13801 | cattgcttaa ggaagtgaac aaaggtagtg agcatcatag ggtgctgagc tgggagcagg |
| 13861 | agggagggaa ggttaggggg cttggtgtct tgatcaaggt gtctggtatt ctgagtcaga |
| 13921 | agtgcattgt ccaagttctg atgctcttct ccaggctcca aaggagactc cagtctcagg |
| 13981 | ggaagtgctg gagaccttag gccctttggt tggattcctg gggacagaga gcacacgaca |
| 14041 | gatcccccta cagatcctgc tgtcccatct cagtcagctg caaggcttct gcctaggaga |
| 14101 | gacatttgcc acagagctgg gatggctgct attgcaggag tctgttcttg ggtatggacc |
| 14161 | ttcgagaact tcagattcta actcattcta tacccagtcc ctcagccacc atcatcagtg |
| 14221 | gcagcctgtt ccatattctt aaggtcccct ggagccctgt gtccgaaatc ctagcatgtc |
| 14281 | ctcttttccc cttccttttc ctcacagttc cctcagctcc ccagcccccg attttcttcc |
| 14341 | tgtccccagg aaaccagagt tgtggagcca ggatgaagta gagcaagctg gacgcctagt |
| 14401 | attcactctg tctactgagg caatttcctt gatccccagg gtgagatgaa ggaagaaggg |
| 14461 | aagggagtaa atgcatagag gggactggtg agctggttat ggggacccgt ggccaaagag |
| 14521 | ggcaaaggat atgaagccta gatctggggg gagactgcaa aacagagaca ggactttgga |
| 14581 | cttagagcta tagcagcagg tcctgatctg tccagatctc cccactctcc ttctaccttc |
| 14641 | tcatgcagga ggccttgggt ccagagaccc tggagcggct tctagaaaag cagcagagct |
| 14701 | gggagcagag cagagttgga cagctgtgta gggagccaca gcttgctgcc aagaaagcag |
| 14761 | ccctggtagc aggggtggtg cgaccagctg ctgaggatct tccaggtgaa actacccaaa |
| 14821 | tacttatatg tccagcagga tgtacaggga gtatcaaacg gtctgggttc tacatgtgct |
| 14881 | cttccctggg actgggtttt ctaatttata aagcaaagag tttagaggga tgatcttcaa |
| 14941 | gcctcttgta gttctagaat tctgtagttc tgggagtttg taaactatta agttttcttt |
| 15001 | tagcccagaa cttccatttt cctgctctct cgtgtctgct ctagactcag ctctagctcg |
| 15061 | gctaagtgtg gagctctctg ctggggagat ccctagaagc tttgaaggag acattgtgag |
| 15121 | gctggagaac tgggttcaaa ttcagtgcta ccattaaatc tctgaataac atcctcagtc |
| 15181 | ttccatctat aaaagtcttg gcatctccaa tcacttcttg ttctattatc tcctaagccc |
| 15241 | tatacatatt actctgtaat actcctttga tccctatttc tcacagtgct ctatcctcca |
| 15301 | aaggttggaa gactcactct atctacagat atctctctgg gcatatttta ttactgcgct |
| 15361 | gacctcctgg ccctgccttc ccccttcaga acctgtgcca aattgtgcag atgtacgagg |
| 15421 | gacattccca gcagcctggt ctgcaaccca gattgcagag atggagctct cagactttga |
| 15481 | ggactgcctg acattatttg caggagaccc aggacttggg cctgaggaac tgcgggcagc |
| 15541 | catgggcaaa gcaaaacagg ttagggatgg agagccaact ggggttggcc atgaggaagc |
| 15601 | tatttgggtg tgatgtagga cacaaagaga atggagagtt ggatgagagg tgggggaagc |
| 15661 | aagagataga agagttagaa gatttgggtc acaagtagga ggtgaaggga gataaatatt |
| 15721 | gaggaaagag agctagtata atgaatagag ggacgaaagc agtggttacc aaattttaat |
| 15781 | gcatatcacg atcatcaagg gaacagattt ttttctttat ttttttttct ttcttaaaaa |
| 15841 | aataatggca tgcttcggct gggtgcagcg gctcacgcct ataatctcag aactttggga |
| 15901 | ggccaaggcg ggcagatcac gaggtcagga gatcaagacc atcctgtcta acacggcgaa |
| 15961 | acacggtctc tactaaaaat acaaaaaagt tagccgggca tggtggtgca cacttgttgt |
| 16021 | cccagctact tgggaggctg aggcaggaga atggcgtgaa cctgggaggg ggagcttgca |
| 16081 | gtgagccgaa gtcaagccaa tgcactccat cctgggtgac agagcaagac tccatctcaa |
| 16141 | aaaaaaaaaa aaaaaaaaaa ggcatgcttc atgaatttgc gtgttatcct tgcacaggcg |
| 16201 | ccatgcaaat ctctgtatca ttccaatttt ttggggtatg tgctgctgaa ctgagcatgg |
| 16261 | gaacagtgcc agtgccagat taccatgctt cactgactta ataaaaacct ttggggaggc |
| 16321 | tgggcgcagt gactcatgcc tgtaatcaca gcactttggg aggcggaggc aggtggattg |
| 16381 | cttgagccca ggagttagag accagactgg gcaacatggt gaaaccctgt ctctactaaa |
| 16441 | aatagaaaaa acattagctg ggtgtggcgg cacatgcctg taatcccagc tactcaggag |
| 16501 | gcbggggtag gagaatccca tgagtgcagg aggtggaggg tgcaatgtgc caagatcgca |
| 16561 | ccactgccct ccagcctggg tgtcagagca agaccctgtc tcataaatta aaaaataagc |
| 16621 | ctctggggga aagagtctag acatctgcat ctcctttttt tttttttttt tttttttttg |
| 16681 | agacagagtc tcactctgtc acccagcatc caggctggag tgcagtggtg tgatcttggc |
| 16741 | tcactgtaac ctctacatcc tgggttcaaa cgatcctcct gcctcagcct ctcaagtagc |
| 16801 | tgggactaca ggtgcaccac acctggctaa tttttgtatc tttggtagag atggggtttc |
| 16861 | actatgttgc ccaggatggt ctcgaacttc tgggctcaag caatcctccc acctcagcct |
| 16921 | cccaaagtgc tgggattaca gcbgttagcc actgtgctgg gccctaggca tctgttttaa |
| 16981 | taagcgtctc tgtgtctgat gcacataaaa gtgtggaact catggactag agttagtttg |
| 17041 | ctcttctttt ccactgattg taatgtcttt caaaacacct tagaggaact gtaaggcaac |
| 17101 | ggtctcattt tatagtggag gaaactaaag aaaaggcaaa tgatttacct agagttatac |
| 17161 | agctaagggc agaggcaaga cttaaaaccc agcagtatga ctcccaatcc actgcttttc |
| 17221 | cactcacatt gttcctgtct ttctcctagt tgtggggtcc cccccgggga tttcgtcctg |
| 17281 | agcagatcct gcagcttggt aggctcttaa taggtctagg agatcgggaa ctacaggagc |
| 17341 | tgatcctagt ggactgggga gtgctgagca ccctggggca gatagatggc tggagcacca |
| 17401 | ctcaggtaac acttttcctc ctccctacgg cttcccaaac acccatccca cagacccagc |
| 17461 | cctatagatc atctaaagcc caaggaattt ttttcctgtg accctacctg gtccttcttt |
| 17521 | ctatcttttg ttgatacccc atactagtga ccttcaggac tctgatttat tcactctgag |
| 17581 | gccctggaca cataatactg tctcctacct cttttcctgg aggcttcctc tttttctttc |
| 17641 | cttttctttt ctgagtcctc agccttcccc atgactcctt aggtcttaat agtaacagaa |
| 17701 | tataacccag taacacctat cacttccctg tccattaatt ctccataact ttcctccttc |
| 17761 | ccctcttctc ccacccccca ccccagctcc gcattgtggt ctccagtttc ctacggcaga |
| 17821 | gtggtcggca tgtgagccac ctggacttcg ttcatctgac agcgctgggt tatactctct |
| 17881 | gtggactgcg gccagaggag ctccagcaca tcagcagttg ggagttcagg tcatttgtga |
| 17941 | aggggctgag ggtggtggtg ctgaggtaaa ggtggactta ctggggaaag aaggatcatg |
| 18001 | aaggtctggt cccatggagg aagggaactc atttgaagcc atctcttcct ttgtctcatg |
| 18061 | accacagccc ctttcactga agccgaattc ttcttccttc cttcctactg ttctacagcc |
| 18121 | aagcagctct cttcctcggc accctgcatc tccagtgctc tgaggaacaa ctggaggttc |
| 18181 | tggcccacct acttgtactg cctggtgggt ttggcccaat cagtaactgg gggcctgaga |
| 18241 | tcttcactga aattggcacc atagcaggtg gggagctggg ccactgctgg tgcaagttgg |
| 18301 | tttggtttct ataccatggg tggactggat ggaagactgc cctgcaattc ttaaggtggg |
| 18361 | ggcctgaggg tgtttaaata aggggctaga gacatattgg ggaaggtcta tgatagggca |
| 18421 | ctttgggagt agttagagaa ggtctatagg tttgaagaga gggaaggtca gtctaagaca |
| 18481 | atgtttggat gccacttgct tcaacagctg ggatcccaga cctggctctt tcagcactgc |
| 18541 | tgcggggaca gatccagggc gttactcctc ttgccatttc tgtcatccct cctcctaaat |
| 18601 | ttgctgtaag tattaatgga ctggggtgac cacaggagag ccagggccca atggggacta |
| 18661 | catgcatgca ctgattccta cccctgccct caggtggtgt ttagtcccat ccaactatct |
| 18721 | agtctcacca gtgctcaggc tgtggctgtc actcctgagc aaatggcctt tctgagtcct |
| 18781 | gagcagcgac gagcagttgc atgggcccaa catgagggaa aggagagccc agaacagcaa |
| 18841 | ggtgagttcc cagctgcaca gcttgatcct ccatctcctg acccagaatc aaacccctaa |
| 18901 | tttggtgctg tctggctctt agagtgcacc cagggagatc cctggagtga aggagtctac |
| 18961 | aggcagagcg ctaatttcca agtatcaatg ctcctggaga gctgagttgt gatattactc |
| 19021 | ccattccctg tctattatag gtcgaagtac agcctggggc ctccaggact ggtcacgacc |
| 19081 | ttcctggtcc ctggtattga ctatcagctt ccttggccac ctgctatgag cctgtctcta |
| 19141 | cagtagaagg agattgbggg gagagaaatc ttaagtcata atgaataaag tgcaaacaga |
| 19201 | agtgcatcct gattattttc agaagctgat gaggaata |

The nucleotide sequence of the human STRC pseudogene is shown in SEQ ID NO: 38.

| Human Stereocilin Pseudogene 1 (STRCP1) | |
|---|---|
| (SEQ ID NO: 38) | |
| 1 | gccctgccct cacctggcta tcccacacag gtgagaataa ccagaactca cctccggtac |
| 61 | cagtgttcac ttggaaacat ggctctcagc ctctggcccc tgctgctgct gctgctgctg |
| 121 | ctgctgctgc tgtcctttgc aggtaagaag aacagtgagc agaactgggg atgaggagga |
| 181 | gggtggctgg aaaaagactt taagaatatg gaggtgaacc tgttagatag aaggacaaag |
| 241 | gagagaggca gagacttgtg caaaagggaa aaatgagggt taagaaaagc aggccaagac |
| 301 | ttactgtagg ccagtgaaag gggttcagct caccatcccc tcacctcatc tttagatcca |
| 361 | ggtagggaac tgtgctcagg ggcagggttg agtttgggct ctgtgttcct ctccttcagt |
| 421 | gacctctggt ttctctcctt acagtgactc tggcccctac tgggcctcat tccctggacc |
| 481 | ctggtctctc cttcctgaag tcattgctct ccactctgga ccaggctccc cagggctccc |
| 541 | tgagccgctc acggttcttt acattcctgg ccaacatttc ttcttccttt gagcctggga |
| 601 | gaatggggga aggaccagta ggagagcccc cacctctcca gccgcctgct ctgcggctcc |
| 661 | atgattttct agtgacactg agaggtagcc ccgactggga gccaatgcta gggctgctag |
| 721 | gggatatgct ggcactgctg ggacaggagc agactccccg agatttcctg gtgcaccagg |
| 781 | caggggtgct gggtggactt gtggaggtgc tgctgggagc cttagttcct gggggccccc |
| 841 | ctaccccaac tcagccccca tgcacccgtg atgggccgtc tgactgtgtc ctggctgctg |
| 901 | actggttgcc ttctctgctg ctgttgttag agggcacacg ctggcaagct ctggtgcagg |
| 961 | tgcagcccag tgtggacccc accaatgcca caggcctcga tgggagggag gcagctcctc |
| 1021 | actttttgca gggtctgttg ggtttgctta ccccaacagg ggagctaggc tccaaggagg |
| 1081 | ctctttgggg cggtctgcta cgcacagtgg gggcccccct ctatgctgcc tttcaggagg |
| 1141 | ggctgctccg tgtcactcac tccctgcagg atgaggtctt ctccattttg gggcagccag |
| 1201 | agcctgatac caatgggcag tgccagggag gtgagtgtgg ccagggctgg gactgggatg |
| 1261 | tggcagggca aggaaagtga aattggggta gttttcttcc ttactctttc cctcctaggt |
| 1321 | aaccttcaac agctgctctt atggtaagta acaggagacc agttctgagg gattgggcct |
| 1381 | ggaaaatctg gaggtgaaga gctgaagacc tcagcctcta gagaggaaaa ctgatgggag |
| 1441 | gagtgtagtt tagtggtttt ggggtgtgac tgtctgggtt ggtgtcccag ctccacctct |
| 1501 | tcctagccat atgaccttga gcaggttaca tagtctttct atacctcagt ttccccattt |
| 1561 | ataaaatgag aatgataata ttagttacca cagagttgtt gcacccggtt aaatgagttg |
| 1621 | atactgtgta tgcaaacgac ttaaaaccgt gctggcacat agcgcttaat aatgttagct |
| 1681 | agtaaagatg ggatttggaa aataaggaca cagctggatt cctctacccc cttactactt |
| 1741 | cagtacaaca atgccagaca gtagttagac atattgagtt gctgagcaga tttcctaaca |
| 1801 | tgaggcccgc tgagggttgt gtttaagcta tctaaaagca tatgaagaaa ggagacagaa |
| 1861 | gggggccagg tggacagaaa gaattccaac tggggcttct cctaggtgat tttggacctt |
| 1921 | ggcagggcag ctttctcttt tttgccccgt tgcagcattt caaccagtaa cgcctaaact |
| 1981 | ctcagggacc tcgcttgtag aaaagcctat gcttgccatg ccccttgagg gctctgagtc |
| 2041 | agggtcagaa tcttcagctg gaggaaatgt gaactgacca gatcctgcct gctcctccct |
| 2101 | ctgcacccag gggcgtccgg cacaaccttt cctgggatgt ccaggcgctg ggctttctgt |
| 2161 | ctggatcacc acccccaccc cctgccctcc ttcactgcct gagcacgggc gtgcctctgc |
| 2221 | ccagagcttc tcagccgtca gcccacatca gcccacgcca acggcgagcc atcactgtgg |
| 2281 | aggccctctg tgagaaccac ttaggcccag caccacccta cagcatttcc aacttctcca |
| 2341 | tccacttgct ctgccagcac accaagcctg ccactccaca gccccatccc agcaccactg |
| 2401 | ccatctgcca gacagctgtg tggtatgcag tgtcctgggc accaggtgcc caaggctggc |
| 2461 | tacaggcctg ccacgaccag tttcctgatg agtttttgga tgcgatctgc agtaacctct |
| 2521 | ccttttcagc cctgtctggc tccaaccgcc gcctggtgaa gcggctctgt gctggcctgc |
| 2581 | tcccaccccc taccagctgc cctgaaggcc tgccccctgt tcccctcacc ccagacatct |
| 2641 | tttggggctg cttcttggag aatgagactc tgtgggctga gcgactgtgt ggggaggcaa |
| 2701 | gtctacaggc tgtgcccccc agcaaccagg cttgggtcca gcatgtgtgc cagggcccca |
| 2761 | ccccagatgt cactgcctcc ccaccatgcc acattggacc ctgtggggaa cgctgcccgg |
| 2821 | atgggggcag cttcctggtg atggtctgtg ccaatgacac catgtatgag gtcctggtgc |
| 2881 | ccttctggcc ttggctagca ggccaatgca ggataagtcg tgggggcaat gacacttgct |
| 2941 | tcctagaagg gctgctgggc ccccttctgc cctctctgcc accactggga ccatccccac |
| 3001 | tctgtctgac ccctggcccc ttcctccttg gcatgctatc ccagttgcca cgctgtcagt |
| 3061 | cctctgtccc agctcttgct caccccacac gcctacacta tctcctccgc ctgctgacct |
| 3121 | tcctcttggg tccaggggct gggggcgctg aggcccaggg gatgcbgggt cgggccctac |
| 3181 | tgctctccag tctcccagac aactgctcct tctgggatgc ctttcgccca gagggccggc |
| 3241 | gcagtgtgct acggacgatt ggggaatacc tggaacaaga tgaggagcag ccaaccccat |
| 3301 | caggctttga acccactgtc aaccccagct ctggtataag caagatggag ctgctggcct |
| 3361 | gctttagtgt gagtgctctg ccagagggaa agctcctaga acagtgagaa ggccctccag |
| 3421 | gggaattcct cgaatactca gaggcagtag tgtggggtag tagttgaagc acacagctct |
| 3481 | agagtcagac aggcttggat tcatatcttg gttctgtgac cagccttgaa tgagttattt |
| 3541 | aacttctctg agcaatattt ttctcgtctc atttataaac tagggatgat aatggtatat |
| 3601 | gagataatac atgcbgtggg cttagcacag tgcatgatac acaaacatgc aataaatatt |
| 3661 | accttgttat tcttttgggc tctttgactc tctcactttc tgcaccagaa agaaaaagga |
| 3721 | tcaagttaga ggactctaaa tttttcccct agagagtgag aattggaggc tggcagaata |
| 3781 | caggaagata aggtaggaat gagaaagatt cagggacact accaatcaga agactttggt |
| 3841 | tctaggttca actgtgccac aaattagtgt gatcttaggc aagcaatttc atttagtttt |
| 3901 | tctgggcttc agtttttagt ctgtagaatg gaggggtgag aatatgttaa acaccataat |
| 3961 | taattcactg agtgcctatt atatgcaagg cactttgcta ggttctgtag gatatataaa |
| 4021 | gatttcttac tccatgttgg ggccaccttt ttcaaaccct gggcccagta aaatggaatt |
| 4081 | agatagtctc atagtatttg gttcaggtct acaagtatta attgagccaa ctatggacct |
| 4141 | ggcatgggag agggtacaag agaaattaga gatatgatcc cggacctaaa agagcttaat |
| 4201 | atctgaagaa tcacacttga gatgatggac aagcatccca gcaagtggag ttggaatgcc |
| 4261 | tgggggagct gcaggagaga cagagaagac agctctgttg gcatattgtc tttcttccca |
| 4321 | ccagcctgtg ctgtgggatc tgctccagag ggaaaagagt gtttgggccc tgcagattct |
| 4381 | agtgcaggta acaggtggag ggcacatggg tgggcbgggt gacagccatg gctggaggtc |
| 4441 | cctgccccgt gaggtgaggc catacccacc atgacctcct attcgcaggc gtacctgcat |
| 4501 | atgcccccag aaaacctcca gcagcbggtg ctttcagcag agagggaggc tgcacagggc |
| 4561 | ttcctgacac tcatgctgca ggggaagctg caggggaagc tgcaggtgag cactgagaaa |
| 4621 | ggggagcaag ggcacctgga gcctagtgtt cagagggctt gctttagtgg gaggaggaac |
| 4681 | tccagagagg aaatggcagg gatactgagc atctccagag gcagaatcca ttcctgtgcc |
| 4741 | cctacaggta ccaccatccg aggagcaggc cctgggtcgc ctgacagccc tgctgctcca |
| 4801 | gcggtaccca cgcctcacct cccagctctt cattgacctg tcaccactca tccctttctt |
| 4861 | ggctgtctct gacctgatgc gcttcccacc atccctgtta gccaacgaca gtgtgtaagg |
| 4921 | ttcttgcact actcctcctg ctcctgtcac ggtcaggcca accgcatcca cctggagcag |
| 4981 | ccccttccgg agctcctctc tgtttttttc tttcatgcca gataggcaat gtgccaacat |
| 5041 | cgtagcaagg tttgagagag gcacatctca cgcctgagtg tgaaaaccca atcattatgc |
| 5101 | taatgaacta caaaaggatc agagagctcc tctctattaa aaccagggag aggatgggcg |
| 5161 | tggtggctca tgcctgtaat cccagcacgt tgggagcccg aggcaggtgg atcactaggt |
| 5221 | ccgcctagtg agttcgagac cagcctggcc aatatggtga aaccctgtct ctattaaact |
| 5281 | acaaaaatta gccaggcatg gtggtgggcg cttgtagtcc cagttactct ggaggctgag |
| 5341 | gcaggaggat agcttgaacc tgggaggcag aggttgcagt gagccaagat cgtgccactg |
| 5401 | cactccagcc tgggtgacag agcgagactc cgtcttaaaa aaaacaaaaa acaaaacaaa |
| 5461 | acaaaaaaac agggagagtc tccttcctat ctagacagca gggctacaga gggtcagagg |
| 5521 | aaaacagttt ggaggaagac aaagggttaa gacccatgac tcctcgcagc ctggctgcca |
| 5581 | tccgggatta cagcccagga atgaggcctg aacagaagga ggctctggca aagcgactgc |
| 5641 | tggcccctga actgtttggg gaagtgcctg cctggcccca ggagctgctg tgggcagtgc |
| 5701 | tgcccctgct cccccacctc cctctggaga actttttgca gctcagccct caccaggtat |
| 5761 | gagaatcatc ttctttactt gactggccca tcttctgcta gtggggacaa agagtcaatg |
| 5821 | gcatgtctct cagtggcccc tccctgcaag aaccctatag tgaccccagt gcgagctaac |
| 5881 | cttccccatc tcagatccag gccctggagg atagctggcc agcagcaggt ctggggccag |
| 5941 | ggcatgcccg ccatgtgctg cgcagcctgg taaaccagag tgtccaggat ggtgaggagc |
| 6001 | aggtacgcag gtgagttgtt gtgggatcag taaccaaggc aagagtggaa gaggtagaga |
| 6061 | gaggaaggca cagcbgtcac gcbgggtcgg tgttctagga agaaaggggc aagagagtag |
| 6121 | gcagtggcct caggcagcat agagttccag gagagaggtc tatagatggt gcccctgtgt |
| 6181 | agtggtgtag tgtcagagtg cccagtgtat gtacccatac catctgctgc caggcctgcc |
| 6241 | ttagtgctag tcttggggac cacacaaagg tcagcttcat gccctcctca ggcttgggcc |
| 6301 | cctcgcctgt ttcctgagcc ctgaggagct gcagagccta gtgcccctga gtgatccaac |
| 6361 | ggggccagta gaacgggggc tgctggaatg tgcagccaat gggaccctca gcccagaagg |
| 6421 | acgggtgagc ccctcagcac aagcctacaa gactttaggc ttcccctggg tctgtgtgga |
| 6481 | tggctttccc attgtgtcaa cttgagcaca gtggtgccag cccccatccc acttttgcaa |
| 6541 | cctccattcc ttactccatg gccattctta cctgttacca cctcttcctg gcccttctct |
| 6601 | atctggtctg tagcacccca aacataccct ttgccatttt gaacctaatc tactccagtc |
| 6661 | caatccctag ttccaaaccc tagcccaggc cctgggaaat tcagatgtgg gattagagag |
| 6721 | gaagttcaag gttcatctgt cttttctctc cagtcctaaa ccttctttgg ttacaggtgg |
| 6781 | catatgaact tctgggtgtg ttgcgctcat ctggaggagc ggtgctgagc ccccgggagc |
| 6841 | tgcgggtctg ggcccctctc ttctctcagc tgggcctccg cttccttcag gagctgtcag |
| 6901 | agccccagct tagagccatg cttcctgtcc tgcagggaac tagtgttaca cctgctcagg |
| 6961 | tttgcctgtc tcactccctg gcatgtaccc tccatccccg cttgagcccc agtcaagaga |
| 7021 | atcccattca gggataaaag cagcccctcc tttccctggg tgaacagtag aggtaaactc |
| 7081 | tgtctgcagg aggacgcctt cattcccttt cctcagatca agaagggacc tgagtcactg |
| 7141 | aggatggtta ctggggatgg ttaagaggca gcgggaagtt ttggagggtt tgccttagga |
| 7201 | acccacttag gacctggctg ctgggtcctg agagcbgttg ttttcggtcc catcccaaca |
| 7261 | caggcbgtcc tgctgcttgg acggctcctt cctaggcacg atgtgagtag cagcaacttc |
| 7321 | tcagcctccc gccagaggtc tctatcctct tttaacctgg ctcctgcatc tgcccctcct |
| 7381 | ctctctccgc tcccctcata cttactgcct tgctgcattg tgattgttgt cttccccaac |
| 7441 | acccttccct tcttcttcag gcctcttgtc tctcttgctc tttagctatc cctggaggaa |
| 7501 | ctctgctcct tgcaccttct gctaccaggc ctcagccccc agacactcca ggccatccct |
| 7561 | aggcgagtcc tggtcggggc ttgttcctgc ctggcccctg aactgtcacg cctctcagcc |
| 7621 | tgccagaccg cagcactgct gcagaccttt cgggtatgag agtggcaagg aggatgagat |
| 7681 | aatcagggat accggctctt tctggttggg aggaaggcat cttccctgag gccagggaag |
| 7741 | gcctttcata cctccccact tacacacaca cacacacaca cacacacaca accaattctc |
| 7801 | atgcaggtta aagatggtgt taaaaatatg ggtacaacag gtgcbggtcc agctgtgtgc |
| 7861 | atccctggtc aggtaagtgt gagatctccc aactgagctc ctctccccat tctggggcag |
| 7921 | tttcatatgg ctggtgctac ctcccacact accctgcagt ggccctgaga gttctggtta |
| 7981 | gctctgtgcc cattagcagc cctccccagt gccagatgca ggacagcatg atccactcac |
| 8041 | attgtcctag actaatgtca aagctggaag ggcctgagaa atcttccagg ccacccaccc |
| 8101 | tgctttcaga tgaaaagacc aaggcbggga gaagctaagg gactttgttt gcctggtgcc |
| 8161 | taactagcag caacacttga ccacagcagc ctgcagtgtg aggctcttag gcgtttattg |
| 8221 | ctacagtggc aaatgccatt ccacttctgt cctagctttg gtccctttcc acccccatgg |
| 8281 | ttccttttct ctgagtgcta agtacagact ctctcaccta tcactacact gctataccca |
| 8341 | tcaccgccag cagcctattc ccaccacctg gccagactgc ctgcttcccc tgctcccatt |
| 8401 | aaagctgcta caactggatt ccttggctct tctggcaaat cgaagacgct actgggagct |
| 8461 | gccctggtct gagcagcagg taattctccc cacttaattt cagaacttcc tccctcaatg |
| 8521 | tagtctacct tctttaccta tcccttagcc ctatttggcc agcttatccc tactatcctt |
| 8581 | tatttgattg tttgagatac agtctcactc tgttgcccag gctgcagtgc agtggcatga |
| 8641 | tcagagttcg ctgtaacctc aaactcatga gctcaggcaa tctttctgcc tcagcctcct |
| 8701 | gaatagctag gatgacaggt ggttaccacc atgcctggct aatttttaaa tttttttttt |
| 8761 | gttttttgag atgaagtctt gctctgtcac ccaggcttga gtacagtggc acaagcttgg |
| 8821 | ctcactgcaa cctctgtctc ccgggttcaa gcgattctcc tgcctcagcc tcccgagtag |
| 8881 | ctgggactac aggcactccc cacaatgccc ggctaatttt ttttttgttt tagtagagac |
| 8941 | agggtttcac catattggcc aggctggtct cgaactgctg accttgtgat ctgcctgcct |
| 9001 | ctgcctctca aagtgctggg attacaggtg tgagccacca tgcccggcca atttttaaat |
| 9061 | tttttgtaga gacagacaat acaaaaatgt ggacactatg tggagacact atgttgaggt |
| 9121 | actatgctgt ccagattggt cttgaactcc tggcctcaag caatcctcct gccttggcct |
| 9181 | cccaaagtgc tgggattaca gacctgagcc actgcaccca gccccctagt atcccttata |
| 9241 | atgtgacttg cttttctttt tctttctcct tcccttttct ttcatttctt tctcactctc |
| 9301 | gagagaagag tgggcatctg ggagagtggg aggctggtgg gtcccacaga gtgaggaggc |
| 9361 | aggactgggt ccaaggcagt cctgcctctc cactctaggg ggtatccttg gacagtgtct |
| 9421 | cttctgggaa ggggctcgtc tttctttctc ttgtaggcac agtttctctg gaagaagatg |
| 9481 | caagtaccca ccaacctgac cctcaggaat ctacagtgag taacttgtgt tgagcagtgc |
| 9541 | gctgaattcg accaacattt ttttgagtgc ttactatgtg ccaggcacca tatgatatgg |
| 9601 | aatgggggat atagggatga atgatgcata gtccctgcct cgtggacgtt ctcctagcac |
| 9661 | ctccctttgc cctcctttcc ttccacagtg ccatgcctat cctgactaga gccaaaggac |
| 9721 | tcagaaaacc tggattcagg ttccagtcct gtcacctact tgtcctcttg ggcaagtcat |
| 9781 | ttaacgtccc tgtgtcagtt ttcccttctt taaatgagaa ttacaatggc accagcctca |
| 9841 | taggtagtta ctgtgaagat taaatgaggt aggtcatgta agatatttaa cacagtgttt |
| 9901 | ggtccattgt aaagttccag tagtcatttg ctactgttag tttacttcag gatgacttca |
| 9961 | gaggcactgg ccaagcaaga ataaatagga ataagaaggt atcactttac ttatacccac |
| 10021 | attagaagaa caatgggctt cagaatcttt tttttttttt ttttcgagac agtcttgctc |
| 10081 | tgttgcccag gctggagtgc agtggcgcga tttcggctca ctgcaacctc tgcctcccag |
| 10141 | gttcaagcga ttctcctgtc tcagcctctg gagtagctgg gattacagga atgtgccacc |
| 10201 | atacccagct aatttttgta tttttagtag agatggggtt tcaccatttt ggccaggctg |
| 10261 | gtctcaaact cctgacctca ggtgatccac ccgcctcagc ctcccaaggg cttcagaatc |
| 10321 | taagacatgt ctctagtttc agtttaccac atttctagca gaatgatgtt gggaatgtca |
| 10381 | cctgacttcc ataaatcctt attttctcct ctgataaaca gcagtgatgt tatggggagc |
| 10441 | tgatgagata tctatgtaaa aacatttctc aaaccataaa ttacggtgga tgaacatctg |
| 10501 | tacttgtgtt gagagtactg atatcaagga gcaaacaggc tgttgtatgt gttgaatgag |
| 10561 | cctctcccca ctcacacacc cacagggctc tgggcaccct ggcaggaggc atgtcctgtg |
| 10621 | agtttctgca gcagatcaac tccatggtag acttccttga agtggtgcac atgatctatc |
| 10681 | agctgcccac tagagttcga gggagcctgg tgagagggga tgcctggact ttagtgggag |
| 10741 | cagggaggct gggaccctag gtatagaacc cagctcctat gttctgctct ggcctcactc |
| 10801 | tgcttcccta cagagggcct gtatctgggc agagctacag cggaggatgg caatgccaga |
| 10861 | accagaatgg acaactgtag ggccagaact gaacgggctg gatagcaagc tactcctgga |
| 10921 | cttaccgtaa gtactgcagc tagagatatt ggcccctcag aaagctcaat ctggggtgaa |
| 10981 | gatctgccct tagggaatgc cctggaggag gtagtttttc tgtctggtag ttccctgaca |
| 11041 | taatttatag cccaaagcag aggattttat tcaaagttgc tctatgtatt gactggttcc |
| 11101 | cagaatatgc tccagcacag ggcagctgag ggtggcaaca ctgtattgaa gcctgccaag |
| 11161 | taatcttaca ataacctagt ccacattaat tgagattgag acagagcatc tgaagtgagg |
| 11221 | gaggcaatgc tccaaatctg ccccagagga ttgtagtttg ctcagggcac tgtgttctta |
| 11281 | gtgcattcag aggagtggat cgagagaaaa atatatgaaa aatgtgataa ataccttcaa |
| 11341 | atacctgagg ggctatcaag tagaaattag attgtcatat ttatgagtgg ccccattggg |
| 11401 | caagactaag agtagttaac ggagatcaga tttttacata gtataagaaa aactaaggta |
| 11461 | gtgagttcct ggtccttgga gcbgttcgag cctaagccag atggccccat ggcaggaatg |
| 11521 | ttgtagagca cgttcatata caggttgtgg gaagaaaagg ctataggaac ccaaggctcc |
| 11581 | tccctaccca tggagaaatt tattagtatg ttactcatat gctgcttttc tcattttacc |
| 11641 | cctaccacca ccccgttgcc atccgcactg taagtcagga taggaaaatg ctggtgttac |
| 11701 | agtcttcctg gggaatatgg agctgaagtg gagtaaaagc agttgacttc attcttactt |
| 11761 | ttttcttttt cttttttttt ttttttttga gacagagttt tgcbgtgtca ccaaggctgg |
| 11821 | agtgcagtga cgtgatctcg gctcactgca acctccatct tccaggttca agcaattctc |
| 11881 | ctgcctcagt ctcccgagta gcbgggactg taggtgtgca ccaccatgcc aggctaattt |
| 11941 | ttgtatttgt tgtagggacg agctttcacc atgttggcca ggcbggtctt gaactcctgg |
| 12001 | cttcaagtga tctgcccacc tcggcttccc aacattctta tatttttata ggcctttcca |
| 12061 | cagatttcag ctcttgtatg acttagccca gttccagaac tggtaatcct aggtagggta |
| 12121 | caggttatca cctctgattt cgggtaaaag ggatttattt atttatttgt ttatttattt |
| 12181 | atatttttga gacagagtct cgctctgtca cccaggctgg agtgcaatgg tgccatctcg |
| 12241 | gctcactgca acctctccct ctggggttca agcacttctg cctcagcctc ctgagtagct |
| 12301 | gggattacag gcgcgtgcca ccacacccgg ctaatttttg catttttagt agagacgggg |
| 12361 | tttcaccatg ttgctcaggg tggtctcgaa tttctgaccc tgtgatctgc ctgcctcggc |
| 12421 | ctcccaaagt gcbgggatta caggcatgag ccactgcgtc cggcctgttt ttactttttt |
| 12481 | ttaatgccat tcagatctgt ttaaatatgt gggttctgtg agataattta gaatcccaag |
| 12541 | gttacagatg aggtgaaaga tcctagacca tgcatcaaaa aacttgagtt tctcatttgt |
| 12601 | gaaagaagga taagagaaac acctattttg tctgggtgca gtggctcatg cctataatcc |
| 12661 | cagcatttgg ggaggccaag gtgggtggat cacggaggtc aggtgttcaa gaccagactg |
| 12721 | gccaacatgg caaaacacca tctctactaa aaatacaaaa gttagcbggg cgtggtggca |
| 12781 | cgtgcgtgta attccagcta ttcgggaggc tgaggcacga gaattgcttg aacctgggag |
| 12841 | gtgcgggttg cagtgaactg agatcgcagc accactgtgc tccagcctga gtgatggagt |
| 12901 | gaggccaggt cttgttgtag gatcaaatga gataacacct gaaagaactt tgtaaattgt |
| 12961 | atagcacgta caaacaagaa gggacctctt cacaagcaga ggaagggtgg tcctgtggaa |
| 13021 | aaaaacggga attgggagtg agagacctca acatttgatc tctgtgaacc tcagtttttt |
| 13081 | aatctataaa atggggaaat gttaatggta cttaatattt ggagcttttg agtccattag |
| 13141 | atcaggtagg attgttcgtt attttttttt tttaggaaga ctagaaatat gttgctccct |
| 13201 | ttttctcccc cactcaagct tgatggtggg aattggccct ggagcbgttt actgtcagtt |
| 13261 | cctgtccagc ttcactaaat ttggtctggg gtcacatctt agcbggggac tgtggggttc |
| 13321 | tgtggtccct tctcgacttg gcccagctcc acttgaatga tattgttgtc aaattgctat |
| 13381 | aatagaatcc agttgatgga cagactatcc aatgaatcca ttatgttggt ggtggagctg |
| 13441 | gtgcaaagag ctccagagca gctgctggca ctgacccccc tccaccaggc agccctggca |
| 13501 | gagagggcac tacaaaacct ggtaagagtc caccctacca gactcagatt tgctgccctg |
| 13561 | ggcaattctt gctcctcaga caatgctctc tgactgtcct ccaaccctct acttcttgct |
| 13621 | ttcttgctgc caaacaggtt cctgtctaca aggcctggcc cgttttgcct ctgggttctg |
| 13681 | ttccttgata atatgcttca cgttacttgt ccatacctct tggagtccga gaaatctctt |
| 13741 | ggagtccacc tctcagtctt tctgcctgct cctatctggt ctcattgctt aagaaagtga |
| 13801 | acaaaggtag tgagcatcat agggtgctga gttgggagca ggagggaggg aaggtcaggg |
| 13861 | ggcttggtgt cttgatcaag gtgtctggta ttctgagtca gaagtgcatt gtccaagttc |
| 13921 | tgatgctctt ctccaggctc caaaggagac tccagtctca ggggaagtgc tggagacctt |
| 13981 | aggccctttg gttggattcc tggggacaga gagcacacga cagatccccc tacagatcct |
| 14041 | gcbgtcccat ctcagtcagc tgtaaggctt ctgcctagga gagacatttg ccacagagct |
| 14101 | gggatggctg ctattgcagg agtctgttct tgggtatgga tcttcgagaa cttcagattc |
| 14161 | taactcattc tatacccagt ccctcagcca ccatcatcag tggcagcctg ttccatattc |
| 14221 | ctaagggccc ttggagccct gtgtccgaaa tcctagcatg tcctcttttc cccttccttt |
| 14281 | tcctcacagt tccctcagct ccccagcccc cgattttctt cctgtcccca ggaaaccaga |
| 14341 | gttgtggagc caggatgaag tagagcaagc tggacgccta gtattcactc tgtctactga |
| 14401 | ggcaatttcc ttgatcccca gggtgagatg aaggaagaag ggaagggagt aaatgcatag |
| 14461 | aggggactgg tgagctggtt atggggaccc gtggccaacg agggcaaagg atatgaagcc |
| 14521 | tagatctggg gggatactgc gaaacagaga caggactttg gacttatagc tatagcagca |
| 14581 | ggtcctgatc tgtccagatc tccccactct ccttctacct tctcatgcag gaggccttgg |
| 14641 | gtccagagac cctggagcgg cttctagaaa agcagcagag ctgggagcag agcagagttg |
| 14701 | gacagctgtg tagggggcca cagcttgctg ccaagaaagc agccctggta gcaggggtgg |
| 14761 | tgcgaccagc tgctgaggat cttccaggtg aaactaccca aatacttata tgtccagcag |
| 14821 | gatgtacagg gagtatcaaa cggtctgggt tctacatgtg cttttctctg ggactgggtt |
| 14881 | ttctaattta taaagcaaag agtttagagg gatgatcttc aagtctcgtc tagttctaaa |
| 14941 | attctatagc tctgggagtg tgtaaactat taagttttcc tttagcccag aacttccatt |
| 15001 | ttcctgctgt cttgtctctt taaacagctg actcagctct agctcggcta agtgtggagc |
| 15061 | tctctgctgg ggagatccct cgaaggagac attgtgaggc tagagaactg gtttcaaatt |
| 15121 | cagctctgcc atgaaatccc tgaccaacat cctcagtctt ccatctataa aatgagggac |
| 15181 | ttggcatctc caatcacttc ttgttcaatt cacttctaag ccctatacat cttaacctgt |
| 15241 | aatactccct cgatccctac ttcccacagt gctccatcct ccaaaggttg gaagagtcat |
| 15301 | tccatctaca gatacctcct ggccctgcct tcccccttca gaacctgtgc caaattgtgc |
| 15361 | agatgtacga gggacattcc cagcagcctg ctctgcaacc cagattgctg agatggagct |
| 15421 | ctcagacttt aaggactgcc tgacactatt tgcaggagac ccaggacttg ggcctgagga |
| 15481 | accacgggca gccatgggca aagcaaaatg ggtcagggac ggagagccaa ctgaggttgg |
| 15541 | ccatgaggaa gctgtgtggg tgtgatgtag gacacgaaga gaatagagag ttggatgaga |
| 15601 | ggtgggggaa gcagaagata aaagagttag aagatttggg tcacaagtag aaggtgagga |
| 15661 | gagataaata ttgaggaaag agagctacta taatgaatag agggactaaa gcagtgatta |
| 15721 | ccaaatttta atgcatatca caatcatcaa gggaacagat ttttttcttt tttctttttt |
| 15781 | tttctttctt aaaaaataat ggcatacttc atgaatttgt gtgttatcct tgcgcaggcg |
| 15841 | ccatgctaat ctctgtatca ttccaatttt tttttgtata tgtgctgctg aaccgagcac |
| 15901 | gggaacagtg ccaatgctgg attaccatgc tagactgact taataaaaac ctttgcggag |
| 15961 | gctggacgga gtggctcgtg cctgtaatca cagcactttg ggaggctgag gcaggtggat |
| 16021 | tgcttgagcc caggagtttg agaccagact gggcaacatg gtgaagccct gtctctacta |
| 16081 | aaaatagaaa aaaaattagc tgggtgtggc ggcacatgcc tgtaatccca gctactcagg |
| 16141 | aggcbggggt aggagaatcc cctgagcgca ggaggtggag ggtgcaatga gccaagatca |
| 16201 | caccactgca ctccagcctg ggtgtcagag caagaccctg tctcataaat taaaaaataa |
| 16261 | gcctctggga gaaagtctag acatctgcat ctgctttttt tttttttttt ttttttgaga |
| 16321 | cacagtctca ctctgtcacc cagcatccag gctggagtgc ggtggtgtga tcttggctca |
| 16381 | ctgtaacctc tacatcctgg gctcaaacga tcctcctgcc tcagcctctc gagaagctgg |
| 16441 | gactacaggt gcacaccact acacctggct aatttttgta tttttggtag agatgaggtt |
| 16501 | tcgcbgtgtt gcccaggatg gtcttgaact cctgggctca agcattcctc ccacctcagc |
| 16561 | ctcccaaagt gcbgggatta cagctgtgag ccactgtgct gggccccagg catctgcatt |
| 16621 | ttaataagcg tctctgtgtc tgatgcacat aaaagtgtgg aactcatgga ctagagttag |
| 16681 | tttgctcttc ttttccactg attgtaatgt ctttcaaaac accttagagg aactgtaagg |
| 16741 | caacggtctc attttatagt ggaggaaacc aaagaaaagg caagtgactt gcctagagtt |
| 16801 | atacagctaa ggtcagaggc aagacttaaa acccagcatt ctgactccca atctactggt |
| 16861 | tttccactca cattgttcct ctctttctcc tagttgtggg gtcccccccg gggatttggt |
| 16921 | cctgagcaga tcctgcagct cggtagactc ttaataggtc tgggagatca ggaactacag |
| 16981 | gagctgatcc tagtggactg gggagtgctg agcaccctgg ggcagataga tggctggagc |
| 17041 | tccactcagg taacactttt actcctccct accgcttccc aaacacccat cccacagacc |
| 17101 | cagccctata gatcatctaa agcccaagga attttttcct gtgaccctac ctggtcattc |
| 17161 | tttctgtctt ttgttaatat cccatactag tgaccttcag gactcttgat ttattcactc |
| 17221 | tgaggccctg gacacataat actgtctcct acctcttttc ctggaggctt cctctttttc |
| 17281 | tttccttttc ttttctgagt cctcagcctt ccccatgact ccttaggtct taatagtaac |
| 17341 | agaatataac ccagtaacac ctatcacttc cctgtccatt aattctccat aactttcctc |
| 17401 | cttcccctct tctcccaccc cccaccccag ctccgcattg tggtctccag tttcctacgg |
| 17461 | cagagtggtc ggcatgtgag ccacctggac ttcgttcatc tgacagcgct gggttatact |
| 17521 | ctctgtggac tgcggccaga ggagctccag cacatcagca gttgggagtt taggtcattt |
| 17581 | gtgaaggggc tgagggtggt cgtgttcagg taaaggtgga cttgcbgggg aaaggaggat |
| 17641 | catgaagtta tagtcccatg gaggaaggga actcatttga agccatctct tcctttgtct |
| 17701 | catgaccaca gtccctttca ctgaagccga attcttcttc cttccttccc actgttctac |
| 17761 | agccaagcag ctctcttcct gggcaccctg catctgcagt gctctgagga acaactggag |
| 17821 | tttctggccc acctctttgt actgcctggt gggtttggcc caatcagtaa ctgggggcct |
| 17881 | gagatcttca ctgaaattgg caccatagca ggtggggagc tgggccactg ctggtgcaag |
| 17941 | ttggtttggt ttctatacca tgggtggact ggatggaaga ctgccctgca attcttcagg |
| 18001 | tgtgggcctg agagggtgtt taaataaggg gctagagaca tattggggaa ggtctatgat |
| 18061 | agggcacttt gggagtagtt agagaaggtc tataggtttg aagagaggga aggtcagtct |
| 18121 | aagacaatgt ttggatgcca cttgcttcaa cagcbgggat cccagacctg gctctttcag |
| 18181 | cactgctgcg gggacagatc cagggcgtta ctcctcttgc catttctgtc atccctcctc |
| 18241 | ctaaatttgc tgtaagtatt aatggactgg ggtgaccaca ggagagccag ggcccaatgg |
| 18301 | ggactacatg catgcactga ttcctacccc tgccctcagg tggtgtttag tcccatccaa |
| 18361 | ctatctagtc tcgccagtgc tcaggctgtg gcbgtcactc ctgagcaaat ggcctttctg |
| 18421 | agtcctgagc agcgacgagc agttgcatgg gcccaacatg agggaaagga gagcccagaa |
| 18481 | cagcaaggtg agttcccagc tgcacagctt gatcctccat ctcctgaccc agaatcaaac |
| 18541 | ccctaatttg gtgcbgtctg gctcttagag tgcacccagg gagatccctg gagtgaagga |
| 18601 | gtctacaggc agagcgctaa tttccaagta tcaatgctcc tggagagctg agttgtgata |
| 18661 | ttactcccat tccctgtcta ttataggtcg aagtacagcc tggggcctcc aggactggtc |
| 18721 | acgaccttcc tggtccctgg tattgactat cagcttcctt ggccacctgc tatgagcctg |
| 18781 | tctctacagt agaaggagat tgtggggaga gaaatcttaa gtcataatga ataaagtgca |
| 18841 | aacagaagtg catcctgatt attttcagaa gctgatgagg aata |

### Clarin 1 (CLRN1)

The CLRN1 gene encodes “clarin 1” (CLRN1), a protein that is expressed in hair cells of the inner ear (e.g., inner ear hair cells, outer ear hair cells) and in the retina.

The human CLRN1 gene is located on chromosome 3q25.1. It contains 7 exons encompassing ˜47 kilobases (kb) (Vastinsalo et al. (2011) Eur J Hum Genet 19(1): 30-35; NCBI Accession No. NG_009168.1).

Various mutations in the CLRN1 genes have been associated with Usher syndrome type III (e.g., Usher syndrome type MA (MIM #606397) (see, e.g., Fields et al. (2002) Am J Hum Genet 71: 607-617, and Joensuu et al. (2001) Am J Hum Genet 69: 673-684) and retinitis pigmentosa (see, e.g., Khan et al. (2011) Ophthalmology 118: 1444-1448). Usher syndrome type III-causing mutations have been predominantly found in exon 3 of CLRN1. Usher syndrome type III-deafness can be modeled by generating CLRN1-deficient mice (see, e.g., Geng et al. (2017) Sci Rep 7(1): 13480). Exemplary mutations CLRN1-associated with Usher syndrome type III include: T528G, M120K, M44K, N48K, and C40G.

Exemplary mutations CLRN1-associated with retinitis pigmentosa include L154W and P31L (see, e.g., Khan et al. (2011) Ophthalmology 118: 1444-1448).

Additional exemplary mutations in a CLRN1 gene that have been detected in subjects having hearing loss and methods of sequencing a nucleic acid encoding CLRN1 are described in, e.g., Fields et al. (2002) Am J Hum Genet 71: 607-617, Joensuu et al. (2001) Am J Hum Genet 69: 673-684, Adato et al. (2002) Europ J Hum Genet 10: 339-350, Aller et al. (2004), Clin Genet 66: 525-529. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype CLRN1 protein is or includes the sequence of SEQ ID NO: 39, SEQ ID NO: 41, SEQ ID NO: 43 and SEQ ID NO: 45. Non-limiting examples of nucleotide sequences encoding a wildtype CLRN1 protein are or include SEQ ID NO: 40, SEQ ID NO: 42, SEQ ID NO: 44 and SEQ ID NO: 46.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 39.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 41.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 43.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 45.

| Human Full-length Wildtype CLRN1 Protein Isoform D | |
|---|---|
| (SEQ ID NO: 39) | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISVALWLPATRHQAQGSCGCLVMILFASEVKIHHLSEKIANYKEGTYVYKTQSEKYTTSFWVIFFCFFVHF | |
| LNGLLIRLAGFQFPFAKSKDAETTNVAADLMY | |
| Human Wildtype CLRN1 Isoform D cDNA | |
| (SEQ ID NO: 40) | |
| atgccaagccaacagaagaaaatcattttttgcatggccggagtgttcagttttgcatgtgccctcggagt | |
| tgtgacagccttggggacaccgttgtggatcaaagccactgtcctctgcaaaacgggagctctgctcgtca | |
| atgcctcagggcaggagctggacaagtttatgggtgaaatgcagtacgggcttttccacggagagggtgtg | |
| aggcagtgtgggttgggagcaaggccctttcggttctcattttttccagatttgctcaaagcaatcccagt | |
| gagcatccacgtcaatgtcattctcttctctgccatccttattgtgttaaccatggtggggacagccttct | |
| tcatgtacaatgcttttggaaaaccttttgaaactctgcatggtcccctagggctgtaccttttgagcttc | |
| atttcagttgccctttggctgccagctaccaggcaccaggctcaaggctcctgtggctgtcttgtcatgat | |
| attgtttgcctctgaagtgaaaatccatcacctctcagaaaaaattgcaaattataaagaagggacttatg | |
| tctacaaaacgcaaagtgaaaaatataccacctcattctgggtcattttcttttgcttttttgttcatttt | |
| ctgaatgggctcctaatacgacttgctggatttcagttcccttttgcaaaatctaaagacgcagaaacaac | |
| taatgtagctgcagatctaatgtactga | |
| Human Full-length Wildtype CLRN1 Protein Isoform A | |
| (SEQ ID NO: 41) | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISGSCGCLVMILFASEVKIHHLSEKIANYKEGTYVYKTQSEKYTTSFWVIFFCFFVHFLNGLLIRLAGFQF | |
| PFAKSKDAETTNVAADLMY | |
| Human Wildtype CLRN1 Isoform A cDNA | |
| (SEQ ID NO: 42) | |
| atgccaagccaacagaagaaaatcattttttgcatggccggagtgttcagttttgcatgtgccctcggagt | |
| tgtgacagccttggggacaccgttgtggatcaaagccactgtcctctgcaaaacgggagctctgctcgtca | |
| atgcctcagggcaggagctggacaagtttatgggtgaaatgcagtacgggcttttccacggagagggtgtg | |
| aggcagtgtgggttgggagcaaggccctttcggttctcattttttccagatttgctcaaagcaatcccagt | |
| gagcatccacgtcaatgtcattctcttctctgccatccttattgtgttaaccatggtggggacagccttct | |
| tcatgtacaatgcttttggaaaaccttttgaaactctgcatggtcccctagggctgtaccttttgagcttc | |
| atttcaggctcctgtggctgtcttgtcatgatattgtttgcctctgaagtgaaaatccatcacctctcaga | |
| aaaaattgcaaattataaagaagggacttatgtctacaaaacgcaaagtgaaaaatataccacctcattct | |
| gggtcattttcttttgcttttttgttcattttctgaatgggctcctaatacgacttgctggatttcagttc | |
| ccttttgcaaaatctaaagacgcagaaacaactaatgtagctgcagatctaatgtactga | |
| Human Full-length Wildtype CLRN1 Protein Isoform C | |
| (SEQ ID NO: 43) | |
| MQALQQQPVFPDLLKAIPVSIHVNVILFSAILIVLTMVGTAFFMYNAFGKPFETLHGPLGLYLLSFISGSC | |
| GCLVMILFASEVKIHHLSEKIANYKEGTYVYKTQSEKYTTSFWLTKGHS | |
| Human Wildtype CLRN1 Isoform C cDNA | |
| (SEQ ID NO: 44) | |
| atgcaggccctgcagcagcaaccagtttttccagatttgctcaaagcaatcccagtgagcatccacgtcaa | |
| tgtcattctcttctctgccatccttattgtgttaaccatggtggggacagccttcttcatgtacaatgctt | |
| ttggaaaaccttttgaaactctgcatggtcccctagggctgtaccttttgagcttcatttcaggctcctgt | |
| ggctgtcttgtcatgatattgtttgcctctgaagtgaaaatccatcacctctcagaaaaaattgcaaatta | |
| taaagaagggacttatgtctacaaaacgcaaagtgaaaaatataccacctcattctggctgactaaaggcc | |
| acagctga | |
| Human Full-length Wildtype CLRN1 Protein Isoform E | |
| (SEQ ID NO: 45) | |
| MPSQQKKIIFCMAGVFSFACALGVVTALGTPLWIKATVLCKTGALLVNASGQELDKFMGEMQYGLFHGEGV | |
| RQCGLGARPFRFSCYFLDPFMGLPTGVPHLLSLPCSTSCRREHTSERVQEPAGCFSAVRSKLHAGPAAATS | |
| FSRFAQSNPSEHPRQCHSLLCHPYCVNHGGDSLLHVQCFWKTF | |
| Human Wildtype CLRN1 Isoform E cDNA | |
| (SEQ ID NO: 46) | |
| atgccaagccaacagaagaaaatcattttttgcatggccggagtgttcagttttgcatgtgccctcggagt | |
| tgtgacagccttggggacaccgttgtggatcaaagccactgtcctctgcaaaacgggagctctgctcgtca | |
| atgcctcagggcaggagctggacaagtttatgggtgaaatgcagtacgggcttttccacggagagggtgtg | |
| aggcagtgtgggttgggagcaaggccctttcggttctcatgctattttcttgaccccttcatgggactccc | |
| aacaggggtaccccatttactcagcctgccctgctcaacctcttgcaggagggagcacacgagtgaacgag | |
| tgcaggaaccagctggctgctttagtgctgtgaggagtaaactccatgcaggccctgcagcagcaaccagt | |
| ttttccagatttgctcaaagcaatcccagtgagcatccacgtcaatgtcattctcttctctgccatcctta | |
| ttgtgttaaccatggtggggacagccttcttcatgtacaatgcttttggaaaaccttttga | |
| Human Wildtype CLRN1 Genomic sequence (NCBI Accession No. NG_009168.1) | |
| (SEQ ID NO: 47) | |
| 1 | aggagatact tgaaggcagt ttgaaagact tgttttacag attcttagtc caaagatttc |
| 61 | caattaggga gaagaagcag cagaaaagga gaaaagccaa gtatgagtga tgatgaggcc |
| 121 | ttcatctact gacatttaac ctggcgagaa ccgtcgatgg tgaagttgcc ttttcagctg |
| 181 | ggagctgtcc gttcagcttc cgtaataaat gcagtcaaag aggcagtccc ttcccattgc |
| 241 | tcacaaaggt cttgtttttg aacctcgccc tcacagaagc cgtttctcat catgccaagc |
| 301 | caacagaaga aaatcatttt ttgcatggcc ggagtgttca gttttgcatg tgccctcgga |
| 361 | gttgtgacag ccttggggac accgttgtgg atcaaagcca ctgtcctctg caaaacggga |
| 421 | gctctgctcg tcaatgcctc agggcaggag ctggacaagt ttatgggtga aatgcagtac |
| 481 | gggcttttcc acggagaggg tgtgaggcag tgtgggttgg gagcaaggcc ctttcggttc |
| 541 | tcatgtaagt agcaattgca tttgagttat ttaatgcttt aggcagactc ttcccagtgt |
| 601 | tgcgaggaat tatatttgag aattttgccg tgtttactgc aggacttttt aaatcggtgt |
| 661 | gaaccatatg aaaaacctat gactctgagc aatttcttct tcctagtttt tattatttta |
| 721 | tacttgcttt ttattataat atagagttaa ttcattgtta cataattaag gtttttggaa |
| 781 | atattggcaa ttaagatgct taagtattaa tatttatgta aaaaattatg gagtcttttt |
| 841 | aaaaaagtaa acttggggaa ataggaaagc tgtaaagaat gatctttatg ctttttgttc |
| 901 | tttataaaaa gaaccaaggt catgggctcc gtatttaacc aggttgccac ctttctcatg |
| 961 | attttgtttc ctgctcccca ctccctccca ttattcctgc taagaccttt cctgctgcta |
| 1021 | aatattcagt tttcattttt aactaatttg gaatcatttg gctatagaaa tttaaaatga |
| 1081 | tctgcbgtgc taactgggaa agaaatggat gcctatttag tatagaacat tttaaactga |
| 1141 | ttgacctgca aatcatgtag agaatatgag agagattttc ttgttgtgat ttttgtgaaa |
| 1201 | tggaagtgta atccacagta tttataacct gtttatctta agaagagaat ttttaaaaat |
| 1261 | taccatgtga ataggcaact cattaaatga aaattaatag gaagtcattt gttatatctc |
| 1321 | ttacaacaca cattcagaag ttattattat ttcagaaggg ctggtttgga acaaccttat |
| 1381 | gaagacacag tcagtaaatt actgcataaa tcactcttca ggaaaggagg ttaccaactg |
| 1441 | aagcatttaa aatgaattat tattttgccc aggttttttt tttctttcta gtataggtag |
| 1501 | aaggctaaat taattgaatt tattattaac atatgcagtg cctaattaaa tttcagtgct |
| 1561 | ggtctattta tatttctgca acattcctta tatcttctta gcagtcattg gacaccaacc |
| 1621 | ttcagctcac ataggttact aagtgatatg aattttcata gggctccaga aaatttccaa |
| 1681 | gaattggttg ttagcttttt aattgatgaa gtggatacca gttcttttca ctgaatggct |
| 1741 | tttattcatt aaggtaatgg ggcbgttaga gttgcttagt tttcctgggg aaggggaagg |
| 1801 | aagaaaacaa agcagaatgt catgtgatat gcaactgtat taaaaaaccg aaaaggaaaa |
| 1861 | aagttgagag agatgattta accgtgagtc accggcagcc aaagcgtgag taaagcttct |
| 1921 | cacagatgaa tttagacaaa agcggagaag gtactggtga attttctgga gcctttacat |
| 1981 | tttctacagt gaaatggaga taaactttac tcatgccata ggacatgttt caaaacaata |
| 2041 | ataagatgtt ttctgaacac ttactacata ctaagcactt tatatgcttt gtctcattta |
| 2101 | atccttacac agccacattc ttctggggtt tagcgaatga tttttgtggt tgtgtcctat |
| 2161 | gcttgtcctg tctaaggatg aagttgttct aattgggtgc ccctcctttt gctttctgtg |
| 2221 | aggacttgca gaactggtgg ggtttaaaca gtaccctcac ttatctcaca gaatttcatt |
| 2281 | agctcccaga tacccctgac attctccccc tagcctagtg aagaaaatct tccatttact |
| 2341 | tgttcattct gcagtgacag ctccatcaat atacaataga ctatacatat taagtgtact |
| 2401 | gtatatacta tacatgttaa aaatctcatt cattttggtg aggcccagct aagaatactt |
| 2461 | acagtagagc tttttttttt ttcctaagca taaaagtatc tttttcaatg cagcatgaga |
| 2521 | cagagttggg aaaaccaaaa taaatagatc caatggactc cccaaagagg ataatattca |
| 2581 | tttaaataaa cacccctctc agtgttaaaa ctttctaatc aacatgcctt tgggacacat |
| 2641 | tgcaccctca aagtttacac tcccattgca acgcagcttt gtggttcacg ttttttccat |
| 2701 | tcagaatgtc attaccctgt caatgatgtt tcatcaacgt ttgcttggat gagaatcctc |
| 2761 | tgatattctt cctgatagaa atgtataagc cctgttcata taaatgaata aaagatctaa |
| 2821 | ccttactttc tcagtagtgg cttccttgga gcaaaaagca gggacctcca gagagctcag |
| 2881 | gtggatgact cttttctgtt tcttccagag ctcaacttac aattagtgca caattcattt |
| 2941 | cccagaatgt cttctttctt attgtgcctt tagaaagtta ttaagcaaac atttgaattc |
| 3001 | acagaatctt accagtgtaa gaggaatgga aaaggtaact tatcaaggta acaatcactt |
| 3061 | cgtggccagt tttttcggct cactgcaact acccctcctg ggttcaagcg attctcctgc |
| 3121 | ctcagcctcc caagtagctg ggactacagg cgtgcaccac catgcccagc taattttttt |
| 3181 | tttgtatttt tagtaaagac agggtttcac catgbgggcc aggctggtct catggcaagt |
| 3241 | tttctttgtg ttgtcatgtt attatcaatt aataggaatt tatatttcag ttctgttagg |
| 3301 | tggataaaca ctattttgca tacctaaatg tttcatttat atcagcactg gccaataaaa |
| 3361 | atatactata agcaggccgg gtgcagtggc tcacgcctgt aatcccaact tttgggaggc |
| 3421 | caacactttg ggaggacaca gggtcaggag atcgagacca tcctggctaa cctggtgaaa |
| 3481 | tcccgtctct actaaaaata caaaaaatta gccgggcgtg gcgggggcgc ctgtagtccc |
| 3541 | agctacttgg gaggctgagg caggagaatg gcacgaaccc gggaggtgga gcttgcagtg |
| 3601 | agccgagatc tcgccactgc actccagcct tggtgacaga gcaagactct gtctcaaaaa |
| 3661 | aaaaaaaaaa aaaaaaaaaa aaaaaatata tatatatata tatatattac aagccacaag |
| 3721 | ccacatatgt acttttaaat gtcctagtag ccatattaga aaacaaaagt aaaaaggaat |
| 3781 | agatgaaatt aattttaatg atttttttaa acccaagata tccaaaatat tatcatttta |
| 3841 | acatataatt aaaaatttat tgagatgttt tacattgttt ttttttttct tgacgctgtc |
| 3901 | ttggaaatct agagtgtatt ttacatttac attacatcta attcagtcta gccacatttc |
| 3961 | acatgctcat ttttttgtgt gtggttttat ggagcagaga gtttaatagg caagaaagaa |
| 4021 | aagagaaggc agaagaaaat ggctcccctg tacagagacg cgggggtggg gcgctccaaa |
| 4081 | gccaaaagag gaggtcccta agtatggtag acaccagcca ggaatatatg cagtgtctgg |
| 4141 | aggaggggat gtctgatttg catagggtca catgctcatt tttatggcta ctgtattggt |
| 4201 | cagtacagat ttagatgggg atttgctctg aacaagttgg tgattgatgg tgttcatatt |
| 4261 | ttaattgaat ttttcctggg ttctgctgta cacttgtatg tgtgttagtt tcatgtgcaa |
| 4321 | tgcttgtgtc acttttaaaa cccagatata tggcataaca tgagaatgaa aaatggacca |
| 4381 | gaaaaatagt ttggcaatgt agtcatgttt gttcctatta aatgttccct attgaccact |
| 4441 | ctatctcttt taattataac aagaatctgc cctgccagca tgcccagtta cgctgggaaa |
| 4501 | acttctgcct catttactct ggctgattct catccactta tgggtcagtg gttcattttc |
| 4561 | tagaggtcac cagcattcat acctagcata caatttcatt cattataatg aaggaatgtt |
| 4621 | ttcccttcaa agagacacaa ctagtgggct taatttttct tgatatgtca cctgtaaaat |
| 4681 | tttaatgatg atgtttaaac tctaaatgta gccatcaaga caaaaactgc aaattttgag |
| 4741 | cctcagtgtg tgtgggtggg tttctgtttc ggtaatttga aacattgcag aatatcatca |
| 4801 | aaatatgata cccaagaatc atatggtatc aatcattcct agatatactg atctattcat |
| 4861 | tgccaagata gttcaatgag ctggcaaaaa catatggaat tattttctta aaatgtgaaa |
| 4921 | aataaaattt aaccaatcat gtatcacagc ttgcaacttt agtcatactt tgaaaagcat |
| 4981 | tttaatttgg acctcatgat tgaaaattta taaaaagctg aacagaaatt agttcacttc |
| 5041 | atattttaga aaagcagagt ttctttacta aatgaggcat ttgacccaaa ttggagagaa |
| 5101 | aatgttgaaa ctacttctgt gagcaagcag gtggcttctc aaacacatgt tgggatgaaa |
| 5161 | tggttgggcc tcagggtctc agtgcctgtc actgagagtt ggcactctct atctccatgg |
| 5221 | tctcctccaa gtgtgactct tgtctcttgc tgacctgacc tgccccaagt gactcactgg |
| 5281 | tcatgaccct gcacaccttg cgtctctcct atcaccctgc cgatggcaga gctacaaagg |
| 5341 | tctttgatgt agctctgtct gatatcctgt gtttccccct atggtctgtg tggaagcagg |
| 5401 | tatgggggtg tgtaaagggg aagcctatga agttcatctg caaagactac ctggttaggg |
| 5461 | gaggagagga agaagctata tgcaccattt caccagcaag catgggctct tctgcctttt |
| 5521 | agcttagggg tcctgttgtc tagtctcact cacctattaa aacagtccag caaatagagg |
| 5581 | ttttgtttac ctcccattaa aaaaaaagca attaatggaa tagaagataa taatgtatga |
| 5641 | gaagcactat tgtgaaagaa aaaccttcaa cttctctcag ccaaataatt gcttcctcct |
| 5701 | ctgtccttcc cagaccttga tgtttgctct attatttcaa aacaactata ttataaatat |
| 5761 | ttgagaatgt gtatttccct gcaaggagat cttaatcccc aagaaggcag gagctgtgta |
| 5821 | ttattcatcc cagtgctcct tcaaaccagg gcctcagaca gtgcatggcc caaagaggta |
| 5881 | cttaataaac gtttggtaaa ctgacactat tgaaattaag caacctggat ttgaggtggg |
| 5941 | tctctgccac tcacaagaga ttacgctttg agaaaattcc atcacttcat tgattttcag |
| 6001 | ttcttgcatc tgtatatggg agacgatact aggtgatttc tgacatctct caccgtttaa |
| 6061 | atgctctgtg atctatacaa cgaggggctc gcbgttctag acaagttcct tccagcttta |
| 6121 | cagttgcata acccttctaa tcttagtcac atgatgactc cactgacaga tttttggcca |
| 6181 | ccatcattag acatgctgag ttacgtgtgc ctttgctctg atcctcaaaa ctcatgattt |
| 6241 | ttaaagtttt ctgaaatatc taccatttat caggatccag atggatttca tgaccaaagt |
| 6301 | ggatgtttct tttctctccc attacaatct tttacttttt gtgtgggaaa ttgcatgtta |
| 6361 | aagaaaggga aattgaagaa tgggatgctt tggaattctg gcaagatgga ttagtgggtt |
| 6421 | ccagaaagta ggggcagcca caaataccga aataaatgag atcgtattat tgagaaagca |
| 6481 | caaatggaag aaggtcaaaa gcaagaagaa gctgacatcc tgcttcctcc aatttttgct |
| 6541 | ttctctgttt ttccaagaaa ctcctcttcc aagccttgct gaaaaactcc actttcctaa |
| 6601 | atctaacttc ttaaactgat aatggcaaga agttaggaat gaccaatatg tttaacactc |
| 6661 | caagagtatt tgttttgttt tgttttgaga ctgggtctca ctctgtcgtc cagcctggag |
| 6721 | tgcagtggtg tgatcacagc tcactgcagc cttgacctct cctgcccaag gaatcctccc |
| 6781 | acctcagcct cctgagtaga agggaccaca ggcatgtgcc actacacctg gctaactttt |
| 6841 | aaaaaatttt tttgtagaga tggggttgcc caggcaggtc tcaaactgct gggctcaagc |
| 6901 | aatcctcctg cattggcctc ccaaagtgct ggaattatgg gcataagcca ctacgcctga |
| 6961 | cctctccaag ggcattcttt acccagaaga ggaacttggc agaacttatc ctccaattgg |
| 7021 | tgaggaatat ggagaaaatg actttaagca aaggaacttc tggttctgcc tacctaatcc |
| 7081 | agaaaaagaa gttttatttc tcccttcccc tagtaactat cttcccatat tcacataaaa |
| 7141 | aagtacagaa tcaacattgt tcaagaatta taattttact tgtaagcaca tgtgcacacg |
| 7201 | cacacccata taccttcctt ccctttaaat catcccacac cctaatagta gtaaaatcat |
| 7261 | tgacccgagc atacctggga gaggaagagg agtctgacag gggcaggttc taagtggcac |
| 7321 | tcctggaact taaccctggt gtatatgaac tttacctatt gaaggatgac tcctcaactg |
| 7381 | ttctcacaat ttgctgctct gctttctttt ctaatttctg aaggtgactc atcttcccca |
| 7441 | aggactttca gacttctcag aagaaaaaaa tattgggtgg gtctctgcca ctggcaaaag |
| 7501 | attagacttt gagaatcata aaagtatatc agtatatact cattaatatt gaattactat |
| 7561 | aattaatatt atgatattga tataatgata gaatgatatt gataaaagca atattcaata |
| 7621 | atgaatatta tttcagctgc ccacttattg ggtgcctcat aggtgccagg cattttgtat |
| 7681 | gtattatcta caacccttac atgggacata ttatgatgct gtttctcttg aagaaatatg |
| 7741 | gaaactggaa acagagaggt caccacaatt ttccaaagtc acatagctaa taggtagcag |
| 7801 | acttgggatt caaattcata tgcatatggt aaatcatgct cttcctctgc tacattttgc |
| 7861 | ccccttagaa tatgaaaaag ggatacaaag agatgaagaa aatatgtaag attatccttc |
| 7921 | aatttcacta tcttttttaa agtttttttt attatacttt aaattctgaa atacatgtgc |
| 7981 | agaacatgca ggtttgttac ataggtatac acgtgccatg atggtttgtt gcacccatca |
| 8041 | acctgtcatc tacattaggt atttttccta atgctatccc tcccctagtc ccccacccac |
| 8101 | cgacaagccc cggtgtgtga tattcccctc cctgtgtcca tgtgttctca ttgttcaact |
| 8161 | cccacttatg agtgagaaca tgcggtgttt ggttttctgt tcctgcgtca gtttgctgag |
| 8221 | aatgatggtt tccagcttca ttcatgttcc tgcaaaaggc atgaactcat tttatggctg |
| 8281 | cataccattc tatggtatac atgtgccaca ttttcttaat ctagtctatc attgatgggc |
| 8341 | atttggctta gttccaagtc cttgctattg tgaacagtgc tgcaataaac atatgtgggc |
| 8401 | atatgtcttt atagtagaat gatttataat cctttgggta tagacccagt aatgggattg |
| 8461 | ctgggtcaaa tggcatttct ggttctaggt ccttcaggaa ttgccacact gtcttccaca |
| 8521 | atagctgaac tagtttacac tcccaccaac agtgtaaaag cgttcctatt tctccacatc |
| 8581 | ctctccaaca tctgttgctt cctgactttt taatgattgc cattctaatt ggagtgagat |
| 8641 | ggtatctcag tgtggttttg attagcattt ctttaatgac aagtgatgat gagctttttt |
| 8701 | tcatgtttgt tggccgtata aatgtcttct tttgagaagt gtccgttcat atcctttgcc |
| 8761 | cactttttaa cggggttttt tcttgtaaat ttgtttaact tccttgtaga ttctggatat |
| 8821 | tagtcctttg tcagatgggt agattgcaaa aattttcttc cattctgcag gttgcccgtt |
| 8881 | cactctgata atagtttctt ttgctgcgca gaagtttttt tagtttaatt agatcccatt |
| 8941 | tgtcaatttt ggcttttgtt gccattgctt ttggtgttta gtcatgaagt ctttgcccac |
| 9001 | gcctatgtcc tgaatggtaa tgcctaggtt ttcttctagg atttttatgg ttttaggtct |
| 9061 | tatgtttaaa tctttaatcc atcttgagtt aatttttgta taaggtataa ggaaggggtc |
| 9121 | cagtttagtt ttctgcatat ggctagccag ttttcccaac accatttatt aaatagggaa |
| 9181 | tcctttcccc gttgcttgtt tttgtcaggt ttgtcaaaga gcagatggtt gtagatgtgt |
| 9241 | ggcattattt ctgaggcctc tgttctgttt cattggtctc tgtatctgtt ttgatacaag |
| 9301 | taccatgctg ttttggttac tgtagacttg tagtataatt tgaagtcagg tagcgtaata |
| 9361 | cctccagttt tgttcttttt gcttaggatt gtcttgacta ttcaggctct tttttggttc |
| 9421 | catatgaaat ttaaagtagt tttttctaat tctgtgaaga aagtcaatgg tagcttgatg |
| 9481 | ggaaaagcat tgaatctata agttactttg ggcagtatgg ccattttcat gatattaatt |
| 9541 | cttcctatcc gtgagcatgg aatgtttttc catttgtttg tgtcctttct tatttccttg |
| 9601 | agcagtagtt tgtagttctc cttgaagagg tccatcacat cccttgtaag ttgtattcct |
| 9661 | aggtattttg ttctctttgt agcaattgtg aatggaagtt cactcataag tttgctctct |
| 9721 | gtttgtctgt tattggtgta taggaatgct tgtgattttt gcacattgat tttgtatcct |
| 9781 | gagactttgc cgaagttgct tatcagctta aggagatttt gggctgagac gacagggttt |
| 9841 | tctaaatata caatcatctc atctgcaaac agagacaatt tgacttcctc tcttcctatt |
| 9901 | tgaatacgct ttatttcttt ctcttgcctg tttgccctgg ccagaacttc caatactgtg |
| 9961 | ttgaatagga gtgttcacca cctattttaa gaatagtatt gaagcctcac aaaagctggt |
| 10021 | tctcatgtaa ccatctgaga atatttggcc ttatgacttg aattcattca ttgccttttt |
| 10081 | atttcacatt ttagtgatcc tgatgtctaa atcttaatct ttgatccttg caaggtaaaa |
| 10141 | tagccaagtc aagcctgttt aataatattg gttgaggaag tcacatgctt atgatcaatc |
| 10201 | tttgggttat gtaattatat taccttaatg ttggcagttt aggtgtaagg cagagatatc |
| 10261 | tgatcacatg tgtggttagc taatttaaga tcactgccaa ctaaaatctt catggtatga |
| 10321 | tcttcaaagt tagctacttt gaccacagca atgatttcac cacagcaatt aacaaaatgg |
| 10381 | cagactcttt cctgaggtgg catgaacagt tttaaaacaa agtcaaggac caaaagaaaa |
| 10441 | gcaggcacat ggcatttgat tcatttcaaa aactagtatt gtattaagag ccaaggggat |
| 10501 | agaattgtag catcaattaa aatcttgttt gaaaaaaaat aaaaacaaac gtccattttt |
| 10561 | atctctcaaa tatattaggg ttttcataaa gttataggtg tatttttaaa aaaacaaaac |
| 10621 | tcatatacat tagactgaaa aatttgcctg tcatctcatc atgcagctaa atgcaattgt |
| 10681 | ctatggcgag atacactctt attagaggta tgatagcact aactaatagc aaactttgta |
| 10741 | cctggtagtc taatttatgc agggttcata tttcgctccc tctcagcatg ctgtaactgt |
| 10801 | ggcaaagcca ttctcaggag ttattacccc aacataatca tacccctgtg gattaggagc |
| 10861 | agttaaatgg gtcctgttat cagagacaca tatgtgccac agccgcbgtc atccctaagc |
| 10921 | caccgtgggt gattaagact cactgatggg actacctctg aataggcttg agtgaggtgg |
| 10981 | atacacttca gctgagagaa attcaggtaa ggggctgaga aaatcagatt ttgaatggtt |
| 11041 | ttatcatacc atcaggtctc cttttaagtg ctggggtcat ggatttcatt caacctgacc |
| 11101 | acatagcctt tggaagcttg gctcaatacc tgagtgtgag attatgggtg atattaaagg |
| 11161 | agatgaatgc attgagctga tgtcagagaa tgtcttttac tggattttca taatgactgg |
| 11221 | ctgcagatgg gctgagggga aagtcagatc aagagcattt ctgtaagaag aaagaaatct |
| 11281 | tccctcttat tctctttcaa gaaaatgaat agctgagcac caagaggcca aatacttttt |
| 11341 | taaaaaacac atccttttat gtagagaagg acaggttgag acgaaaaaca ggacctctga |
| 11401 | aactgtcttt atagctttaa tctaggaaga aattgcggca ccattgctga catcattatc |
| 11461 | agaggctgcc ttagttctga gagcttcaca gatggccttt tctgcatttt acatctggcc |
| 11521 | agatgaaggc aaaaaggttg atgaaaccaa aactattaga tcagtggtcc ataactctgg |
| 11581 | ctgcacttta gaatcatctg gggagtcctt aaaactactg atcctgaggc tccatcccag |
| 11641 | accaattgaa tcaggatctc tgggggtggg acctgagcat tggaatgctt taaaagttcc |
| 11701 | tcagggactc taatgtgcag ccaaggctga gaatgactga ggtggatggt ggccaagaca |
| 11761 | ggtgaggcca agagttagaa gcccttactg ttagggaagg cagaagccac aaggaggagg |
| 11821 | ggagggggaa ggagcagtat ttagcatttc ttccacacag ttggggggtt ttctgatcaa |
| 11881 | aattaggctg ggatctttcg cttccatttt catgaaggtc ttatgctttg tccacagcca |
| 11941 | cctggcctca gggcaatgag caatctgatt gatgaatttt cagtaagaaa ctgagcacac |
| 12001 | ttggctctca gccccagtgg cttcccctgt ttccaaatct gcccaccagt tacaggagcc |
| 12061 | tgctcaccaa ctggttgggt taaagaagtc ggctctgtcc ttggcaggga ggccttcagc |
| 12121 | tgtctggccc tgtctgtgac tgcgtgggtg aagctgccta atttggggaa cttgatggaa |
| 12181 | gatctaagct atgttctcta acagttttat cagaaataaa gttaactttt gacctccatc |
| 12241 | tgcctgtctc ctgtggaagg gctctgctcc ttccaaagag actctcaggg gttctcctta |
| 12301 | gaggtgtgtt atcagtccaa agatcatttt agaccagcca ttacagagga tgtccaagaa |
| 12361 | attgcacaag ggaaatagaa gtaagaatga gagcaatata tcagatagta aggaaagtat |
| 12421 | gtgactggtt tcaaataagt aattaaccat aaatccaaag ttcctgcatt agttatagca |
| 12481 | atagtcatcc acctggtcag gaaaaaaaaa gtaaaagact gagctgcaag atagaaagtt |
| 12541 | tgcctgaatt ccacgtactt caaggactac tatggaggat tccttggtcc caatgcagag |
| 12601 | acatgtactc tgaccaccca tgggccaaat ccctcttacc caccctcagt gattcctcct |
| 12661 | gggacctcac tacattgagt tcttacattc cccactcttt tcgggggaaa tataaccctt |
| 12721 | ctgctcttct catgatgtta aaaatattta gacaattact taaaatttac aacaatcttc |
| 12781 | cacataaatg atctcactta tgtttcttgt gagctctgtg atttatttct attatcatct |
| 12841 | atgctgatag ttaaggaaac tgagatatcg agaccaacat gcatagtaaa tggcaaaacc |
| 12901 | agatgaattt taaactttcc taactccaaa agccacatcc tgcccaaccc gccatgctgc |
| 12961 | cgctcagtta atgcctggct gtttgtctcc ccattggccc ctctacccat tgtgctttga |
| 13021 | tggcacactg tattccaact gcctgagact cctggttaat gccattacgt acagacttag |
| 13081 | gttgaattta ctaggatttt taagatttgt aggataagag atatgactgt tagactggaa |
| 13141 | tcagcaatag ataaaaggtt aacaaagttt cagaataaaa tataatagaa aaccccagca |
| 13201 | gatagaagta aaatgaatgg taagacaact gaaatgaaag ccatacattt agaaatatca |
| 13261 | aataaaacac agattaatgg cagacaataa aggaacatac ttagcagtta gtaaaaacac |
| 13321 | tttttacctt attcttatta ccctccagta accttttttt tttttttttt tgagacaaag |
| 13381 | tctcgcbgtg tcitccaggc tggagggtag tgatgtgatc tcggctcact gcaacttctg |
| 13441 | cctcttgggc tgaagagatt ctcctgcccc agcctcctga gtagcbggga ctataggggc |
| 13501 | ccatcactgc acctggctaa tttttgtatt tttagtagag acggggtttc accatgttgg |
| 13561 | ccaggctggt cttgaactcc tggcctcagg tgatccaccc gcctcagcct cccaaagtgc |
| 13621 | tgagattaca agcgtgagcc accaagcccg gctgtaacct attgaaaata acattacttg |
| 13681 | acatgtgaga caaattattt gtaagttaaa gagtttatgt gccttcaatg ctcccaccct |
| 13741 | tccctcccct aaaaagatta ttgagtgccc atgatgtgcc taagccttct ggtagactct |
| 13801 | gagaatgtga agagagttag aggatacttg ttcaacaacc cagaatctag cagaagtgtt |
| 13861 | ttgaaatgac cagccacttg ggagagctat gctagcatat cattcaggat gggctgggtc |
| 13921 | atgttttaat aacaagtaag tttgaacttt ataggtttga aacaaaaaag atgtgtttct |
| 13981 | tgctcacagt tcatattttt tggagattgg ctggagcttc attccaggat actgccacca |
| 14041 | tctggaatgt tgacagttac cgtaacagag aaagagttgt ggaggcbgtc acactggcaa |
| 14101 | ttaaattcgc tgacctggaa ctgacacatg ccacctccac ttatatttta ttggccaagg |
| 14161 | cttgttacac agccacatct aacttcagag aggtcaagat gagcaaatcc taccacgagc |
| 14221 | tatagatgga tgagtagcac tcattattat cacaattatt ttatttgtta taaaaactcc |
| 14281 | aagaaggaga ggtactctat gtgaggaata ggcataggaa aatcagaggg aagttcttaa |
| 14341 | agatgagcta gatttcacta gatggcattg aagaaacatt ccaagtaaat gaacagcata |
| 14401 | agcaaatgca tgaagaactt attgtggtcc tagtacagac gatgcgtgag agtgtggagg |
| 14461 | agggaaaagg taagactgga gaggaaggca ggaaccagaa caggacagac tgtgttcact |
| 14521 | gtcaggcagt taagtctatc ttgtaggcaa catgaagcct tttaaggaag gcaagcagct |
| 14581 | atgtgacagg acagaagatg gattggaaga aaatcaaaag agaagtgggg accagtcata |
| 14641 | aggctcctcc aatatttggg gatccaaacc aaagcactgg cagagaaata gaaaggaagg |
| 14701 | gaaatatttc caaaatattc aaaacagaaa ccaagagaac ttgatgacag aagatgaacc |
| 14761 | caggtttcta ctgaatggac agttgttaca ttctctgaga taaggaatac agaaggaaaa |
| 14821 | agttgagagg gaatatgaaa ttatttttaa tcatgtttaa tttgatcact tgtggaacat |
| 14881 | caaccagaga tgtccatcag ttacaccaat ttgtaggttt tgagagaggc ctgaggtaga |
| 14941 | ggcagagaag tgggaatcag cagattagcg gcagtagttg gaaccataac tgaagatgag |
| 15001 | atttcccagg gaggggagtt gaacaggaag gtagacaaag gctgagtgca gtggctcata |
| 15061 | cctgtaatcc cagcactttg ggaggctgca gtgggtagat cacaaacaag atcaggagtt |
| 15121 | caagaccagc ctggccaaca tggtgaaacc ccgtctctac taaaaaaaca caaaaattag |
| 15181 | ccgggcgtgg tggcacacac ttgtaatccc agcctcagga ggctgaggca ggagaattgc |
| 15241 | ttgaacccgg gaggcagagg ttgcagtgag ccaagatcgt gccattgcac tccagcctgg |
| 15301 | gcgacagagc gagactccgt ccccaccgac cccctaaaaa agaaagtaga caaagatgtg |
| 15361 | tcctagaaaa cactaatatg aatgggtaga gaggggagac ttgtcaagga gatcgagggg |
| 15421 | agagtcagtg aggtgaaacc ttagaggata gaccttcacc aaggaagtaa cagtcaacag |
| 15481 | gcctaaatgc cacagagatg tcaaatgaga gacactggaa atggttcttt gaatattaca |
| 15541 | gccagaacat cacaggagac catttccaaa gcagtcttga tgaagtggtg gggaagggag |
| 15601 | gtccctgaag gagctaagga gggactggga gatcatgacc cagagataag tgttgcaggt |
| 15661 | ataaaaggga aagactgaga tcaggaaata gccacaggat ccctaaggcc aggcttcggg |
| 15721 | tggggtggtg tgtgtgtgtg tgtgtgtgtg tgtgtgtgtg tataaaatgg gaagaccttg |
| 15781 | agtataattc tgtactaaga agatagagca gtagagagga aaaggctgaa gacagatggg |
| 15841 | agggtaaata gtgaattaga aaggtctgtg caaagatgag aaaggatgag tctgaaagca |
| 15901 | cagggagaag ggccagcctt ggacaggaga aaacatttct ttcactaaga ataaagggaa |
| 15961 | ggttgggtat acaccacaaa gaaggtgtag atgggtggca atggagttct gtcaagttga |
| 16021 | gggcattcca tgatagcctc acctttctct gtgaaatgag agagtttagt ttacaagata |
| 16081 | tttctgagca cttcataagc caggttcata gactgaaaga agtggaggtg gagtgtgata |
| 16141 | ggtccttaag aataggggaa gtttggaata tctgacaagg gacagagagg aaaaaactag |
| 16201 | aaaaggcttt gcagaatgtg ggcccacaga tcagaggcta aggggtcccc atttgtgcag |
| 16261 | gagagtaagg gcagggaggc aaggctcaca gcccaaaata tagagcccct caccaaatga |
| 16321 | ctgcaggagg gcagctttcc tatgagagca tccctatcac tgttttcact ccgagtcatt |
| 16381 | aacttacgac ttactcagct ctgtttcgta atagcagact cgagtaatga gggtatgaca |
| 16441 | gcctctctct gcatgccaag gtatgcagcg tggatttcct ttttcgcttt ctctctcctg |
| 16501 | tggcttaggt gccttctgtt ctgctaccag gatagagaac ccagtgacta gtttcttcta |
| 16561 | gctctctttt tctgactagg tatcttgtca gaaatttctg cttaccagac ttcatggaga |
| 16621 | gggaatcaag ctttgaatca gggttgaaaa agtagagctt aatatatata ttacaaaatg |
| 16681 | ccactcacgt tcttgaggtt accttgtatc tataccacaa ctagcattct tttagaaagc |
| 16741 | accattaccg aagtaatccc tttcctggga attcacccaa aaaggttatt cccacttatc |
| 16801 | ccccatctcc aaaataaaaa agaaaaatgt gtgtgcttag agatgttcct ggaagcatga |
| 16861 | gctgtaatac tgaaccaata gaagacgaac taacagattg cagggcatcc gtttggcaaa |
| 16921 | aaacttatgc agttatctaa atgatagtta tgaagacaat atgtataaca cattatattc |
| 16981 | tgagtgaaaa gaacagaagg tgatttcaaa actgcattgg gataatagta atataggaaa |
| 17041 | tagtgagatg aacaaagatt tcaaaggaac aaaaataaag aaaaattttg ctttttatat |
| 17101 | tggtaggcgt atgggtgaaa ttccaatttt aattttaatt tcatagttat aaaattgttc |
| 17161 | ataaaaaaag gtcccccata gacagttggg ctttgggaca aactaacaga aacagagtag |
| 17221 | gaagaaaatt catcttcctt caatcccctt tctctgctta aaacaaaaca aaaaagagct |
| 17281 | ttgtcatgtt caggtgtgca acgaattctt tttccaaatc tggaacttta catctgctat |
| 17341 | taaacaggag tcagtttcca tgtaacatgt tgacaatccc ccaagtgtgt tggaataatt |
| 17401 | ttttttaatg aggagatttg aaattccatt tcaattgcca acctgcctct ttcaacttct |
| 17461 | aaaaacaaag taaaacaaaa caaaaacaca ctgggtccta tcaccccctc ttgctactac |
| 17521 | tattttatct ccattgccct gaattctttc caaacttctt tccacccagc tttgatttgt |
| 17581 | tttcagtcgg gtttattgag gcataattta cgtacagtaa aattcatcct tcttagattt |
| 17641 | agaggtctat gcattttgac taatgcatat tggcttataa tgactaccac aacaaagata |
| 17701 | tagaacacac ccatcactcc cgggttctcc ctgtcccttt tttggtccat ctcctctcct |
| 17761 | acccccaacc ccttggcaac cactgatctg ttttctgtcc ttatcatttt gctttttcca |
| 17821 | ggatgttgta tataaggaat catgcagcat gcagcctctc gagtctgact tcttccagtt |
| 17881 | agcacagtta tttaagatct atccgagtta ttgtgagtag cagcatcttt tttattgctg |
| 17941 | actagtattt catcacatgg atgggccaca acttgtttat ctgttcacct gtcaatggat |
| 18001 | actaagttgt ttccagtttt tggcaaatat gaataaagta aacatttgca tacagatttt |
| 18061 | tgtgtggaca catgttttca attctcttag gtaaatactg agcaatggga ttgctgggtt |
| 18121 | atatgttaag tctatgttca gttttctaag ttctgaaaca attggatatc tatatgcaaa |
| 18181 | aatacaaaat taaccttgac tcaaacttgt accatacaca aaaaataacc tgaaagagat |
| 18241 | cacaggtgta aatgtaaacc tagaactaaa aacttcaagg agagaaacat agcagaaaat |
| 18301 | atttgtgacc ttgcattagg caaagacttc ttagatttga catttgaaac atgatacata |
| 18361 | aaaaatcttg ataaattgga gttcataaaa ataagaacta ctcttcaaaa gacactgata |
| 18421 | agagaatgaa aatacaagcc acagacagag aaaatatttg tgaatttctt atctgataaa |
| 18481 | gggtttgtat ccagcatgca taaagacttt tcaaaactca ataataaaca atccaataag |
| 18541 | aaatgcacaa aagatacaaa cacatcatcg aaaatgacct atgaaaggca aataagccca |
| 18601 | caaaaaaatt ctcaacatca ttagacactt gggaaatgca aattaaaaca acactgagat |
| 18661 | aaactacata tctattaaat ggctaccact ttaaaaacct gtcaagtgcc agccagaatg |
| 18721 | tggaacaagt aggactctct tacattgcta gtgggaaggt gaatggtaca gccactttgg |
| 18781 | aaaacactcc gcagcttctt atagttacac ataggacctg ggggtagagg atggattgac |
| 18841 | tgtaaagggg caaaacttcc tggtaggggt ggggaaagtt tttagaaggg tgaaattgtt |
| 18901 | ctatatcttg attattgtgg tggttacaca actgtctgca tttgtcaaaa ctcacagaac |
| 18961 | tgtacactaa aaaggtatat ttttatgctc tgctaatttt actttaatct taaaaatagg |
| 19021 | aaggaaaaaa taaaatcaat gccactgtgc gactttgggc aagttacttc acttctctgt |
| 19081 | gccttggtct tttgaaatct atacattaag gataaaataa taccttcctc atactgttag |
| 19141 | aattaaatgt gctaatttat gttttatata tataaagtac ttggccgggt gtggtgactc |
| 19201 | acacctgtaa tcccagcact gtgggaggcc gaggtgggca gatcacttga ggacaggagt |
| 19261 | tcaagactag cctggtcaac atggtgaaac cctgtctcta ctaaaaatac aaaaattagc |
| 19321 | tgggcttggt ggtgcgtgct tgtaatccca gctacttgag tggctgaggt gggaggatca |
| 19381 | cttgaactcc agaggtggag gctgcagtgg gctccaccca ctgcctgggt gacagagcta |
| 19441 | gactccatct cgaagaaaaa ataagtactt gaaacatagc aaatgtttta taattattgg |
| 19501 | cttttttttc ttattgttat tacttgcatt attgcbgttt gaagaagttt ggtgataatg |
| 19561 | gagagaaaag ggcaattagg ggtctgggat ggtttaagta tgaggagacc gagacacatt |
| 19621 | gactcagagt gaagaaatca aagataagag aatgaaagaa agggagggta attctgaacg |
| 19681 | cacagacaaa gttatgttac taacgtggca tcggcbgtgt gttgtaataa ataactccct |
| 19741 | ttcacttgtc aatagctaat caaatacttt ctggagacca gaagtgactt gctggatcaa |
| 19801 | tgacaactcc tccacagatc aaaatgttca aatccttttt ctgtgttgta atcctaaatc |
| 19861 | taaaagaaca gagagaccaa gcaaatctac ctcccaacat cattaaagtg acaactctca |
| 19921 | gtatttattt gaatggtctg ctctcagctt caaccaagga aaagtcaaat tagtgttggt |
| 19981 | cagaaaacag aagggtgtta cgagagttct ggctggtcat tacagacttg gggatttttg |
| 20041 | attaaaagaa gaagaagaag aagaaacctg ataaagtgta aatatagcaa gcagggatta |
| 20101 | gtgtcctgct gggtcatgtt ctcacaacag tgagaatttc agagatttca taagaattaa |
| 20161 | actgctccac atgacaattt attttacctt ctggcttttc caggaggcaa atcagtgcaa |
| 20221 | cttctttctg cctttgtttc aatttggtaa caaccctcaa ttttaggaca ggctaaacct |
| 20281 | agccacccta tcagagatga tgaagtagcc atctttttaa caggtgggga gatgaatgga |
| 20341 | atcagggttt gtttgtttgt ttgtttaata actgctagta aaaaccaagt caatagctga |
| 20401 | ctgagtgtaa gggaggctcc agaaggcagg ttattgtagt atagatgtga ctcgacttat |
| 20461 | gatgatgtta cttcccgata aacccgtcat aagttgaaat atcgttaaat tgaaaatgct |
| 20521 | tttaatacac cgaatctacc gaacatcata gcttagctca gcctacgtta aatgtgctca |
| 20581 | ggacgcacat tgcctacagc tgagccaaat cacctggcaa cacaggacac tgtagagtat |
| 20641 | cggttgctgg cccttgtgat gctgtgactg actgggagct gcgcttagtg cctctaccca |
| 20701 | gcattgagag tttcttatcg cttattacta gcctgggaaa agaccaaaat tcaaaactca |
| 20761 | aagtgcggtt tctaccgaat gcttataact ttcagaccat catgatgttg aaaaatcgaa |
| 20821 | ccatcgtagg ttgggatcca tcctataaga cgagctacac tgccggaagt gtaagactgc |
| 20881 | tatgctgccg gaagatgggg catagtggac aactgcaagt cctgacaaca ggaggtcagc |
| 20941 | atctgcgacc tttaacatcc acattgacac taccacagtc ttccaaacag agctgatgat |
| 21001 | atagtttgga tgtcgtccct gcccaaatct catgtcgaat cgtaatcccc agtgttggag |
| 21061 | gtggggcctg gtgggaggtg attgggtcat gggggcagag ttcttatgaa tggtttagca |
| 21121 | cggtcccccc ttggtactgt atagtgagtg agttctcatg cgatctggtt gtttaaaagt |
| 21181 | gtgtggcacc tcccctctct ctctttctcc tactctggcc atgtgaagtg ttggctcccg |
| 21241 | ctttgccttc caccatgatt gttaaattcc cagaggtctc cctagaagct aatgctgcca |
| 21301 | cgtacagcct ggagaactgt gagccaatta aacctcatct ctttttaaat tacccagtct |
| 21361 | caggccgggc gcggtgactg acacctgtaa tctcagcact ttgggaggct caggcaggaa |
| 21421 | gatgatttga ggtcaggagt tcgagaccag cctggccaac atggtgaaac cccatctcta |
| 21481 | ctgaaaatat aaaaattagc caggcatggt ggcgggtgcc tgtaatccca gctacttggg |
| 21541 | aggctgaggc aggagaatcg cttgaacctg ggagtcagag gttgcagaga gccaaaatgg |
| 21601 | agccactgta ctccagcctg ggcaatggag tgagaccctg tctcaaaaaa tatatatata |
| 21661 | ttacccagac tcaggtattt ctttacctga gactatgaga gaatggacta atatagctga |
| 21721 | agaattttat tttattttta aaaaactttt acgtttgggg gtacctgtaa aagtctgtta |
| 21781 | cataggtaaa ctcctgtcat gaggatttgt tgtacagatt ctttcctgct cctccccctc |
| 21841 | ctcccaccct ccatcctcaa gaagatccca gtgtctgttg tttccttctt tgtgttcgta |
| 21901 | agttctcatc atgtagctcc cacgtataag tgagaacatg cagtatttgg ttttctgtcc |
| 21961 | ctgtgttagt ttgctaagga tgatagcctc caactccatc tatcttcctg caaaagacat |
| 22021 | gatctcattc atttttattg ctgcatagta ttccatggtg tatatgtacc acattttctt |
| 22081 | tatccagtct gtcattgatg ggcatttagg ttgattctgt gtcttcagaa ttgtgaatag |
| 22141 | tgctgcaacg aacattcgtg tgcttgtgtc tttatagtag aatgatttct attcttctgg |
| 22201 | tagtaatggg attgcbgggt caaatggtcg ttctgctttt agctctttgc agaatcacca |
| 22261 | tactgctttc cacagtggtt gaactaattt acactcccac taacagtgta taagtgttcc |
| 22321 | cttttctctg caaccttgcc agcctctgtt atcttttgac tttttaataa aaaccattct |
| 22381 | aattagtgtg atggtatttc attgttgttt tgatttgcat ttctctaatg atcagtgatg |
| 22441 | ttgagctttt tttcatgttc gttggctgca ggtacatctt cttttgaaaa gtgtctgctc |
| 22501 | atgtcctttg cccacttttt aatggggttg tttttctctt gtaaatttaa gttcctcata |
| 22561 | gatgcbgggt attagacctt tgtcagatgt atagcttgca aatattttct cccattctgt |
| 22621 | aggttgtctg cttactcttt tgattgtttc ttttaccatg cagaagctcc taagtttaat |
| 22681 | tagatcccat ttgtcaattt ttgcttttgt tgcaattgct tttggtgtct ttgtcatgaa |
| 22741 | atctttgcca ggtcctatgt ccagaatgat attgcctagg ttgtcttcta gggtttttat |
| 22801 | agttttgggt tttacattta aatctttaat ccatcttgag ttgatttttg tgtttggtgt |
| 22861 | aaggaagggg tccagtttca atattctgca tatggctagc cagttatccc agcattattt |
| 22921 | attgagtaag gagtatctcc tccgttgctt gtttttccca ggtttgttga agatcagatg |
| 22981 | gttgtaggtg tgtggcctta ttttggggct ctctatcctg ttcatttggt ctatgtgcct |
| 23041 | gtttttgtac cagtaccatt ctgttttggt tactgtagcc ctatagcata tttcaaagtt |
| 23101 | gggtaacatg atgcctccag ctttattctt tttgcttaga attaccttgg ccatttgggc |
| 23161 | tctttttggt accatatgaa gtttaaaata gttttttttc tagttatgtg aagaatgtcg |
| 23221 | ttggtaattt gataggaata acatgtaatg atattgattc ttcctatcca tgagcatggg |
| 23281 | atgtttttcc atttgtttgt gtcttctctg atttcttcaa gcagtgtttt gtaactcata |
| 23341 | ttgtagagat tattcacctc cttgcttagc tgtattccta ggtattgtat tctttctgta |
| 23401 | gtaattgtga atgggattgc ttttctgatt tggccctcag cttggtattg ttggtgtata |
| 23461 | ggaatgctag tgattttttg tatcctgaga ctttgctgaa gttatttatc agctgaagga |
| 23521 | gcttttgggc tgagactagg gggtttttta gatatagaat catgttctct gcaaacagat |
| 23581 | ttagtttgac ttcctctctt cctacttgga tgccctttat ttctttctct tgcctgattt |
| 23641 | ccctggccag gacttccagt accatgttga ataggcgtgg tgagagaggg cattcttgtc |
| 23701 | ttgtgccagt tttcagggag aatgcttcca ccttttgccc attcagtacg atgtttgtgg |
| 23761 | tggtttgtca tatatggcta ttattatttt gaggtgtgtt cctttaatac ctagtttatt |
| 23821 | gacagttttt aacatgaagc agtgtttaat tttattaaaa gtcttttctg cctctgttga |
| 23881 | gatagtcatg tggcttttgt ctttagttct gtttatgtga tgaatcacat ttgttgattt |
| 23941 | ccttatgttg gaccaacctt gcatcccagg gatgaagcct acttgattgt ggtggtttag |
| 24001 | ctttttgata tactactgga ttcagtttgc aagtattttg ttgaggattt ttgcattgat |
| 24061 | gttcatcacg gatatcggcc tgaagtttct ttttttgttg tgtctctgtc aggttttggt |
| 24121 | atcagaatga tgctggcctc ctagaatgag ttggggagga gttcctcctc ctcaattttt |
| 24181 | ttggaatagg ttctgtagga atggtaccag ctcttcttta tacatctggt agagtttggc |
| 24241 | tgtgaagcca tcaggtcctg ggatttttta gttggtaggg tatttattac tgattcccta |
| 24301 | aatagaccga taatgatttt agaagtggag tcggtttttt cctggtccag tcttgggaag |
| 24361 | gtgtatgtat ccaggaattt atttagctct tctaggtttt ctagtttgtg tgcatatggg |
| 24421 | tgttcatagt agtttctgat ggttgttttt atttccgtgg gatcagtggt aacattctct |
| 24481 | tcatcatttc tttttttttt tttttttttt ttttttgaga cggagtctcg ctctgtcgcc |
| 24541 | caggctggag tgcagtggcg cgatctcggc tcactgcaag ctccgcctcc cgggttcacg |
| 24601 | ccattctcct gcctcagcct cccgagtagc tgggactaca ggcgcccgct accacgtccg |
| 24661 | gctaattttt tgtattttta gtagagacgg ggtttcaccg tgttagccag gatggtctcg |
| 24721 | atctcctgac ctcgtgatcc gcccgcctcg gcctcccaaa gtgctgggat tacaggcgtg |
| 24781 | agccaccgcg cccggcccat ttctaattgt gtttatttga atcctctctc ttctcttctt |
| 24841 | tattaggcta gctagtggcc tatctatctt attaattttt tcaaaaaacc agctcctgga |
| 24901 | tttcttgatc ttttgaatgg tttttcatgt atcaatcctt cagttcagct ctgattttgg |
| 24961 | ttatttcttg tcttgtgcta gctttggggt tgacttgttc ttgcttctct aattctttca |
| 25021 | gttctgatgt tagtttgtta gtttgagatc taactttttg atgtggacat ttagtgctat |
| 25081 | aaatttaact cttaacactg ccttagctgt gtcccagaga gtctggtatg ttgtatcttt |
| 25141 | gttctcatta gtttgaaaaa acttcttgat ttctgtctta atttaattat ttatccaaag |
| 25201 | tcattcagga acatgttgtt taatttccat gtaattgcat ggttttgagc gattttctta |
| 25261 | gtcttgactt ctatttttat tgtaccgtgg tctgagggtg tttgatatga ctttggttct |
| 25321 | tttgcatttg ctgaggattg ttttatgtcc aattatgtgg ttgattttag agtatgtgcc |
| 25381 | atgtggtgat gagaagaatg tatattctgt tggttttggg tacagagttc tgtagaggtc |
| 25441 | tattagatcc atttggtcca atgttgagtt cagatcctga atatctttgc taatttcctg |
| 25501 | cctccatgat ctaatactgt cagtaaagca ctgaagtctc ctactactat tgtgtgggag |
| 25561 | tctatgtctc tttataggtc tctaagaact tgctttatga atctgggtgc ttctgtgttg |
| 25621 | gatgcatata tatttaggat agttagatct tcttgttgaa ttgaaccctt taccattatg |
| 25681 | taacgccctt ctttgtcttt ttttttcttt gttggtttga agtcttcttt gtctgaaatt |
| 25741 | aggattgcaa cccctgcttt tttctgtttt ctgtttgctt ggtagatttt cctccatccc |
| 25801 | tttattttgg acctatgggt gtccttacat attttatctt tatctatcca tccagccatc |
| 25861 | cagccatcca ttcatccgta tcatttttaa ccaataagga cttttaaaag cgcaaccaca |
| 25921 | acaccattaa cataaccaat aaaatctata acaatgataa aatatcatct aatactcagt |
| 25981 | ccatgtccaa ttttccctcg ctatctcaaa atcgtcttct tagaaatggt ctgttcaaat |
| 26041 | gagatcacat ggacagagga aggcgaacct cacactgtgg ggactgttgt ggggtggggg |
| 26101 | gaggggggag ggatagcatt gggagatata cctaatgcta gatgacaagt tagtgggtgc |
| 26161 | agcgcaccag catggcacat gtatatgtat gtaactaacc tgcacaatgt gcacatgtac |
| 26221 | cctaaaactt aaagtataat aataaaaaat aaaaataaaa aaataaaaag tgaaaaaaga |
| 26281 | aaaaaaaaaa aaaaagaaat ggtctgttca aatcacaaac cagattcaga aacaatagcc |
| 26341 | atacattaca ttttattaat atgtctctta aatttctttt aatctattac agtctttgga |
| 26401 | atttttatgt cttcgtttat ccttccaatt attaaaaaaa aagtattttt gtattcattg |
| 26461 | aatagacaat gcttgcagaa aagtaaaaaa aaaaaaattt agtacaaaaa ggtacatagt |
| 26521 | gagcbgttcc ttagtctccc ttcccagaag caatgttacc acttttgtac aaatagtctc |
| 26581 | tgcctagaca cacatgccag tccctaaggt ggctgtaaca aggtggttaa gagtgagaac |
| 26641 | atgaattcaa attcctatta tgccactcac taagtataaa tcttggtcat ggtacatgcc |
| 26701 | tctgtgcctc agtttttaat aatggtacct acctcatagg gctgttgaga gaattaaatc |
| 26761 | agataagtgc ttaaataact attaatattt attattattc acattccctt ttggcttttt |
| 26821 | tcccaaatag cagagtggtg cacatatgtc ttcatttatt tggcttgttt tttcacctca |
| 26881 | catcacattt tgatgaataa ttccatacat gttgttatag atttgcttca ttctttgtaa |
| 26941 | tcattgacta atattccatt gtatgaatat gctactgcta aacatgtacg ttatttccaa |
| 27001 | cctcttatta tcaagaaatg ctgcaatgaa tatccttgta atactttagt ggattcatgt |
| 27061 | gcaaaaatat tcataggata aaatcctgaa agttaaattg ctgagctaaa gggtatgtgc |
| 27121 | attttaatgc tttatagatt gcccagctgc ctcaaaggag gttataacaa tttacactcc |
| 27181 | caagaaaaat gcacaagggt ccccatttcc ccatacccta gctaacacag gatattgcta |
| 27241 | aatgctttca tctttgtaaa catgatgtat tgaaaatggt atctcaaagt tttaatgtgc |
| 27301 | atttttctga ttgtgagaag ataaaggaaa tgtagtacaa ctaaacatca gcagtcaaat |
| 27361 | gacctggcca tgactcctga gtgaggacac tggtaaacac catcaggatc caaacacctc |
| 27421 | tgtatttacg aagaggatgc tccctattgg atagcactaa gcttatttca tgtatgtaca |
| 27481 | tatgtagtta gttaattaca tccagcggtg gcaaagggct tgttctgacc caatgaaact |
| 27541 | ttctctcctg gcccccttcc agcatgtggt caggagtaga gtgttgtggc catgaggcat |
| 27601 | gcatttgtac agatgactac ttactcctcc ttgaaacatt tttttccatt tgcttccctg |
| 27661 | ctgtctcact catgggtctg ctcctaattc acaaatcact cttttcccag tcttcttggt |
| 27721 | tgggttttct cctcttctgt gcttgtagac atgggggagc cccagggctt ctctcttgaa |
| 27781 | ctacagcttc tccctgggtt catctccttg ggatgtctgt tccaatgggt ttaaatacta |
| 27841 | gggctaggac tagggagagg tcattgaggc acttagcaca taaagtttaa ggaaacattc |
| 27901 | tttatcaggc ccatgcaagt gcaggactgg ccctggaggg tgactaccac cttacctttt |
| 27961 | ccaccctagg caccttgttt gccttaccct agccccagtc ctctttaaca ccccagtctt |
| 28021 | ctccctggac ctccagaaac ataaatccta tttgacattt ctacttggag gttttaaggt |
| 28081 | aactcaaacg taaaatatct aagacagaac tcttgcatca cttcccatcc tggggcccaa |
| 28141 | gcctgtctct tctactagtc tatctcagtt aacagcatca ccatttattc agttgctcag |
| 28201 | gacaaaaaat ttgaagtaat ccttgactct tctttttttt tttttgagac ggattctcac |
| 28261 | tctgttgccc aggctggagt gcagtggtgg gaccttggct cactgcaacc tctgcctcct |
| 28321 | gggttcaagc aattctcctg cctcagtctt ctgacctcgt gatccactcg cctcggcctc |
| 28381 | ccaaagtgct gggattacag gcgtgagcca ccgcacccgg cctggattct tttttttttt |
| 28441 | tttaaacaag tcctatcttc catctccaaa atgtatccca aatctgacaa cctctcccac |
| 28501 | cgtaggccag cccccatctc tcccctctga aaatagcctc ccttagatct ctggacattt |
| 28561 | gttcttcccc acccccttgt gatcactatt cagcattcag aatgatcttt taatattatg |
| 28621 | aaggagactg tgttcctctc ctacttaaaa ttctctagtg gcttcctaat aaatttagaa |
| 28681 | taaaaagcca actcctcgcc atggtcacca ggccaggatc cagtgggtac aacaatctgt |
| 28741 | tcccagcaca ataatcccac ttctgcctcc ccaccattca ccaggctcca ctgcactggc |
| 28801 | tcattcctgc ctcagttgtg cttcttttcc ctggaaagtt ctttctgtag atctttaaag |
| 28861 | ggtgatttcc ttctcaacat tcaggtgtca gctcgtttca ctttcctgac catgcccatc |
| 28921 | cactcagaga tcactcaaaa tcccattacc ctattttatt tctccatcat atgtatcact |
| 28981 | atctgaaact atcttgttgt tgatgcaggc tattttcttg accccttcat gggactccca |
| 29041 | acaggggtac cccatttact cagcctgccc tgctcaacct cttgcaggag ggagcacacg |
| 29101 | agtgaacgag tgcaggaacc agctggctgc tttagtgctg tgaggagtaa actccatgca |
| 29161 | ggccctgcag cagcaaccag gtaggggtgc ctgcaacccc agggccccag agggtgtgtt |
| 29221 | acaatgctct cgtagctctg ccatctgtgg acagcagtgt gttgtcagct cagtgggccc |
| 29281 | tttgcttcat catgtagggt ggctgccctc tgcctgtgag ggcaaagggc cagggtgaca |
| 29341 | gtctttttgg gtacccacaa tttgtgcatc ctgaattctt gtttggtgcc caagaagaat |
| 29401 | ggggtcacac agatgaactg aaggatggtg aatgcagaga attagcaatg aaagtggctc |
| 29461 | tcagcagaga gagaagctga aaagggaatg ggaagggcag gtcactctcc cctgaagtca |
| 29521 | agtcacatct ctccaatgtc cagccaccat ctctgaagtc aagtttcctc tctctgatgt |
| 29581 | ccagccactt ctcctctcta ctggctgagt ctggggtatt tataggcaga ggataggtgg |
| 29641 | tggggcaggc catacataat tttggaaaag gcaacattct attggtaaaa agacattatt |
| 29701 | cataaagaac caattgggaa agagcgggca cacagggatg gaagttctca ctttgggctg |
| 29761 | caggtttcag gcttttcagc tcaaaagtga ggtttttcca gggacctgcc ccgtctgcct |
| 29821 | aaaatttcta cacttctgtc attgataccg ctggataaca gctctcctat aaagttcagg |
| 29881 | ggcttaaaac aaaaatttac taattcaccc ggacagttct tgatagggtc tctcctaact |
| 29941 | gttgcagtta catagcaact gggttggagt catcttttct gggcttgaca tccaggacag |
| 30001 | cttcttccct tgtgtgtctg gtgcctcagt gctcctccag gcagcctttc tctccagaag |
| 30061 | agtagcctgg acttcttggc aactcaagct tccaaaagaa aaaaaagcag agactgctgg |
| 30121 | ttctcttatc aaacaggcct ggaactggca caatgtactt ctgctgccat attcaggagg |
| 30181 | tcaaagcaat cgaaggccaa tccaagttca aaggcattgg agaaaaatga gaagtgtcac |
| 30241 | ttaaatgaga agtgacacat gcgtaaaagg gggaaaagca ttgattgtgg ccatttttgg |
| 30301 | agataagcta tcacgtttat ttgtttgttt gctttctaat ggtctgtctt ctcccattag |
| 30361 | gttataagct ctgtgagaca acaggaatct tgtccatctt gttttatggc tctacttcca |
| 30421 | acacctagaa taatgcctgg cacatagtag gtgctcagtg aataacttaa gcacttgata |
| 30481 | catgtttggg gaactaaaat gaacagaatt aaacttcccc agattggtcc tgccagattt |
| 30541 | gctgatgcca agcatgctga tgcctcacca gatgaaagaa gccctaaaat gtagggtttc |
| 30601 | gctttctctg caaacaagaa aaacttgccc tgaacacaaa atctagaaat agatttggcg |
| 30661 | tgttttctac attgaaatat ttcccgtagt accagaaatt attttcccac agctttgtgc |
| 30721 | tacattaaaa tattgaagtt gactgaaaat atctccattc tttaatcttg gtgtagacta |
| 30781 | gaaacaattt ttttgtaaca aagtaaatat gaaaacttcc taatatttga actccccaga |
| 30841 | tatccccaga tatctccaaa cttaaaatat cattgcaagt taagataaat ttttttaaat |
| 30901 | gactaccgag aaaggtcatt aaaggcttgg ttattaaaat gtacagattt gggttataaa |
| 30961 | gccagaactt atttgtttaa atcattacat atgaccaagc acagaaaata aattacctca |
| 31021 | aatctcctct ttgctaattt ttactggtaa actctataaa atgatcctat ctttaaacct |
| 31081 | ttttgtaaac cccttataag ttagtaagtg agaatgtatt catcagaagg attttagtga |
| 31141 | tgtttgaaat taaaaaagag agatttgatt tttaaaatta tacttgcaga ctactgctaa |
| 31201 | tgaaacttct tctaacccta gttttgtctt atcttcagtt tttccagatt tgctcaaagc |
| 31261 | aatcccagtg agcatccacg tcaatgtcat tctcttctct gccatcctta ttgtgttaac |
| 31321 | catggtgggg acagccttct tcatgtacaa tgcttttgga aaaccttttg aaactctgca |
| 31381 | tggtccccta gggctgtacc ttttgagctt catttcaggt aagtacaaaa ttctacctct |
| 31441 | gaagacaaat gtgcttttca atatgtcaaa aagaccgtct acctaaatat aaagttataa |
| 31501 | tcttaacata tatacatgga tgcacactgt agtattatac ataataacaa aaagtgtgga |
| 31561 | aataccacac ttgctcaaca gtagggaatt caataaatac tttatggaca tctatatgaa |
| 31621 | taactgtgat gctgacatta aattatattt ttgaagatgt aatcaagagg ataaacgctt |
| 31681 | gtctcaaaaa gttacatggg aaaagcagta tgtaaactta tataaacatt gtgaacctaa |
| 31741 | tttgattata tatataaaat atagggaata tacatataaa atacatatat gtatatatgg |
| 31801 | gggctatata tatatatata tgaaagagat agatagatag atagatagat agatagatag |
| 31861 | acagacagac agacaataca gactccaatc tgttggtcgt ggttgtctct gacccatgac |
| 31921 | actatggggg acttttattt ttgctcatac ttttcaatat ttcttagtgt tcaataatgt |
| 31981 | gctattattt atacataata ataaaaataa ataaatggca tcaaaaaaga gtaaagggcc |
| 32041 | agtgttccgc ccacatatga gcagccatat tcaagcctgt agacactttg tgtagcctaa |
| 32101 | tgctaggtgt atctgggcaa ggataaactc taaagccaga aattagttca tcaataaaca |
| 32161 | tgtgctactc aatagctagg gctgatggaa aagaatataa aacccagtct gtgccaaatg |
| 32221 | gtgcttacta tctgcaagtg ggagaaggag aaagacgaga aaatgaaaaa tgtgtgtata |
| 32281 | atttatatgt agcbgttctg taggagatct ctgacttcac cccattctaa ctttgcaaaa |
| 32341 | agatccaaca ctttgtcaga ttcctgggag gcaagtaatt ttattgatgg tttcatggag |
| 32401 | ggatacagaa cgataacaac tcacacaaag caaacaatgt aatgaaaatc tctattcgac |
| 32461 | tgtttctttt tctcctgaag ttgccctttg gctgccagct accaggcacc aggctcaagg |
| 32521 | tactttcttg ctcttgacac tactcccttc tctcatacaa ttcaacccca accacaaacg |
| 32581 | tgtatagatc tctctctcta taaaacaaag gcctgtagtt aacaggaggt cacttgcagt |
| 32641 | gtagcctctg ttcattgtta cttgtgcaca ctgcttaggg tctcacccca tccacattct |
| 32701 | gctaatcaca ttattcaccc atccaatgta gatctctcca gtggagattc tgctaatatt |
| 32761 | ttctttagat ttgtcacaag tatataatac agttttaaat tgtacagatg attatcctat |
| 32821 | aacagaagag tctaagcctt ttacatcttt gtatctctaa cgaaagcatt cagcatgaag |
| 32881 | ctctgtacac agcagacaat tcaatatgaa tttgctgact tgaaacagca agcctagaaa |
| 32941 | ggagatgtta acttggtcac ttagacagaa caggtttcag caatcagaat tcagatgaca |
| 33001 | tggaactggt agaacaggcg ctttgaagca ataggacatg agccagtgag gagagggatg |
| 33061 | gaatatcata aacaaaggcc aagggctttg caatcagagc tgaagagcca agagcacagg |
| 33121 | ctcagggtgt gggcagactg aatgagaaag tgattcaatc acatgtgaaa gtccagatga |
| 33181 | gaagagagag ttgggattac ttctgctcac caaacatcca aaaccaaaca ggtggatccg |
| 33241 | ggtggtgtgc tgttttactg atgaccatta cacagaattt taacagaagg aatgtaaagc |
| 33301 | agtggttctc aaactggagt tcccagatga gcagcgtctg catcatctgg aaacctgata |
| 33361 | aagcagcaaa ttctcaggcc ctaccccaga cccactgaat cagaaacttg ggggtctggg |
| 33421 | ggaagatggc catctgtatt ttaacaatct cccttcagga gattctgagg ctggctcaag |
| 33481 | tttgaactac aggtagttgg ttcaacaggt gttggtggac tgacaaacaa aaagagactc |
| 33541 | cgaggtaact ccaagatggt aatgtcagaa agcagctacc acccctaggg cttgggggaa |
| 33601 | ccaacaaaag agtttggcat tgccagaacc tagaatcttg aggagaggcc ccagagcatt |
| 33661 | gtgtctcaga ccttgaggac ttggcactgg gacaccatga ggggtttctg ggtgtgggaa |
| 33721 | agggctggaa actccccagt tgctgccacc agggagaact acaagtgaag tggaaggtgt |
| 33781 | gggcctttct cccttttctt ttctcgtctt ctctctcccc ctggtgctca tgtttgacag |
| 33841 | aaaacagctg aaaaggcaga actagtttgg ggagtcttga cctggcatca taaagcagag |
| 33901 | aaaagcaaag ctggagtgaa ggtgagacac aacagctcat tagcagcaac agccgtctag |
| 33961 | cgctccagct tctgaatgaa attctgaaga acagcgcact tggaagacaa attatttgac |
| 34021 | agttctgaca gacgaccaaa ctaacagcat ttgaaaagca agatgactca gagaatacag |
| 34081 | aatttaatcc aaatcccaga tcctatctct gcctttggcc aggcctaatg caaggagaac |
| 34141 | ctgagcacac aaatatatgc aggaatgatc aggacctgtg cctgcattct attctgtctc |
| 34201 | acccaccttc aaatttgttg taaaaacatg ggctcaataa aggtttgtga atcagggaag |
| 34261 | gaagagaagg ggagaaagga agggaaggag ccagctccag atctgtgtct tgcagaggat |
| 34321 | aaaggccagt gtttttagat cacccagtgt ttttctaagc ccccaatact tattttgaaa |
| 34381 | tatcaaaatg ttcaataact aaaaaaaaaa ccgttacaac aataaaacat gtttgagggt |
| 34441 | cagatggact ggcagtttgt gacctctggg gataaacagg tcactttgga atcacagact |
| 34501 | tcctcattcc ccttaaatct catatggtac ccagaagccc ttggaacttt ggaaggtgtt |
| 34561 | tattcacagt tgtaatgtcc atgcagaccc tggctctaag acccaattgt gtaagggtag |
| 34621 | gtttgtagcc cttatcccaa acattctaag tgtgagccaa tgcgtcacac actcagaggc |
| 34681 | cagagactgt attggggtcc tttatttcac gtacgagtca cattccatta agagacccca |
| 34741 | gaagtcagct ctcttccact gactggttct cttcccttgt ttctcttgcc aatgtgtgct |
| 34801 | gcccaggtgg cagtgctcac tgtcagcaga gaagaaaaat gctttcctcc ttggacctct |
| 34861 | tttctctttt tctcctccct actcacattc aggttcccta agcttccccg ctccttgtgc |
| 34921 | tgaagtcatt ctatggtcat ttcttcaact gtctacttcc ctgctggatg ggcacccaag |
| 34981 | acttggcatc ctggggcatg tagaaagggg aaagggaagg gaagggaaaa gaagtcctcc |
| 35041 | caattgtcta tctggacctt tccacactgc ccagagtact gctatgggca tctccttatg |
| 35101 | tctcccgatg tggtgcatgc cagaccctgc aggtagaaaa ggaaagaaag caacccattg |
| 35161 | gaccaggcca gcaaaggctt cagtcacaca gctggctcat acttatggct tcatattctg |
| 35221 | ttgcctcttg aaccagacat ttcttccact ctcataacct ccagtttagc tcgtattcct |
| 35281 | cagcattctc catgtaatat tgttgcatga aacccatgca agtcagccaa tttgctcttt |
| 35341 | ctcatcttgt catttataaa ttgatgctga gaagtctttt tcccaaggtt tttagtaata |
| 35401 | ccttcatcat cccccatagt tcattttggg cagtgattcg cttctttgac tgtacattag |
| 35461 | aatcatctga agaactttct aaaactactg atctcaggtc tcacacaaca ctaattaaat |
| 35521 | tatagtctct ggtggggtag ggcttgggca ctgctatttt accttaagct ctctggagtc |
| 35581 | attctaattt gtagccagtg ctaagggttg caatataagt gaatatattt cacgtatttg |
| 35641 | tcaaacattg actgagtgcc cattatgtgc cagccattat aataggcact ggtgatccca |
| 35701 | cagtgaatca ggcacacaat gctgtcttca cggagcttgt tgtctagtgg gagagtcaaa |
| 35761 | caaaagtgta tatcaataat taagtgatta cagattgcaa taattacaat aagggtgata |
| 35821 | aacaggttgc tataatatac aatagtattg cctttcacca gacatttctt aaagaggtaa |
| 35881 | atcatctatc agacaccttt taaaaatctc atctaatttc aaaagtgtac ataaataatt |
| 35941 | aagtgattac agtttgcaat aattacaatg agggtgataa acaagttgct atgaaagaga |
| 36001 | aagatagcag agatctggct ttgagatggt ggtcagggaa gactgctcca atagctgagt |
| 36061 | tctaaagcta agaaggaact gagaaccttc acagaacgtc ccaagtagaa gagaaagcac |
| 36121 | actgaagact ctagggaaag aggtctgctt gttgtaggaa ccgaaagaag gccaatgtgg |
| 36181 | ctaggtgctg ggtagtgagg ggaaatggca caaggaaaaa atgaggttag agagatcagt |
| 36241 | tgataccagt taatgttgga ccctaaacat taaccatggt aagtctttta gaattgattc |
| 36301 | taattgcaat gaaaaccttt tgaaagattt taaaaagata tctgatagct gatttacctc |
| 36361 | tctaagaaat gtctggtgaa agacaatacc atcacattgg agatggaaaa agatgaatgg |
| 36421 | attctaaaaa cattctgaaa gtacattcaa aatgtttttc aggtagctta tgcaactaat |
| 36481 | aaatagtggt ggcattctgg gtaagacaag ggaggagcag gcttgcagtt tagggcaaga |
| 36541 | aaggggtggg gagaagagct cagcactaaa atcatgtgtt ccatttgggg cacatcgagt |
| 36601 | ctgagttgct atgagaccac caagtggaga tgccaagtaa atagtcagtt acatgaatct |
| 36661 | ggagttcagt gaagaggtct agaagaaaga tgtatatttg ggcattattc ggatatagat |
| 36721 | attatgtaaa gcaataaaat tggatgagat cacctaggga gagaatgcac atagataaaa |
| 36781 | actgacctag gaccacttca tgtctaaaac accaaaagca atgtcaacaa aagccaaaat |
| 36841 | tgacaaatgg gatctaatta aactaaagag cttctgcaca gcaaaagaat cagagtgaac |
| 36901 | aggcaaccca caaaatggaa gaaaattttc acaacctact catctgacaa agggctaata |
| 36961 | tccagaatct acagtgaact caaacaaatt tacaagaaaa aaacaaacaa ccccatcaaa |
| 37021 | aagtgggtga aggacatgaa cagacacttc tcaaaagaag acatttatgc agccaaaaaa |
| 37081 | cacatgaaaa aatgctcacc atcactggcc atcagagaaa tgcaaatcaa aaccacaatg |
| 37141 | agataccatc tcacaccagt tcaaatggca atcattaaaa agtcaggaaa caacaggtgc |
| 37201 | tggagaggat ctggagaaat aggaacactt ttacactgtt ggtgggacta taaactagtt |
| 37261 | caaccattgg ggaagtcatt gtggcgattc ctcagggatc tagaactaga aataccattt |
| 37321 | gacccagcca tcccattact gggtatatac ccaaaggact ataaatcatg ctgctataaa |
| 37381 | gacacatgca catgtatgtt tattgcggca ctattcacaa tagcaaagac ttggaaccaa |
| 37441 | cctaaatgtc caacaatgat agactggatt aagaaaatgt ggcacatata caccatggaa |
| 37501 | tactatgcag ccataaaaaa tgatgagttc atgtcctttg tagggacatg gatgaagctg |
| 37561 | gaaaccatca tcctcagcaa actatcgcaa ggacaaaaaa ccaaacaccg catgttctca |
| 37621 | cccataggtg ggaattgaac gatgagaaca catggacacg ggaaggggaa catcacacac |
| 37681 | tggggactgt tgtggggtgg gaagaggggg gagggatagc attaggagat atacctaatg |
| 37741 | ctaaatgacg agttattggg tgcagcacac cagcatggca catgtataca tatgtaccta |
| 37801 | acctgcacat tgagcacacg taccctaaaa cttaaagtat aataataata aaataaaata |
| 37861 | aaataaaaaa acaaaaattg atgtaggacc aattcctgaa gaacactgac agttaatttt |
| 37921 | ttggtttagg aggaggagaa gccagcaaac gacactgagt agcaatatcc aaagaaaaag |
| 37981 | aggaaaaagg aaaactggga gattatgagt gtcccagagg gaatgtttca agattaccat |
| 38041 | cagcagtgag ctttgtgtaa aggtggcctc ctgtaataga ggtgcgggca ggagaaggca |
| 38101 | gaatagggaa aagggggtga aaaagcttcc ctcaagattt ataatacagt ggaagagaga |
| 38161 | gacagagaga gagaaagaga aagagagaga gagaacttaa ggaggtagag gaagagagag |
| 38221 | aaccaaaaaa gagggagctg agtatagaag caattagatt catagttttt agttgcggca |
| 38281 | gtgatatttg agtgggggcc ttttatatat tccattctag gtgtttccca gttgatggga |
| 38341 | gagggtctta cctagatctg catgtaaaag ggagtaggcc agctggcaga cttgacatgg |
| 38401 | atcagtggta gaacatccta gcagttctgt gaatactctc tgagaatgac atgaaaggta |
| 38461 | ttggttcagg ccttttggag gtgataaaaa ccaccaaaat gtggacttat tgcaaattgt |
| 38521 | atttgttacc atttgcaatg attataatta ttctcttata tataggcttt cttacatata |
| 38581 | gcttctctta cacatagcgt catgagttat gccttctctt acatatagtg tctgctatga |
| 38641 | gttatgccaa gcagggcaaa caaaattgct gcctttcttt aaaaagagga cgctcctagt |
| 38701 | atgggcctaa ttaattatga taattacagc tatggcatgg aacataagca cattcatata |
| 38761 | cacaaagaca ataaaataaa gaacagttca aaaacagaac agttacatta tatatcagtt |
| 38821 | tcagtatgaa taataccctg gactctgaaa tatgtctggg gcacattatt ctgtaattgg |
| 38881 | tggtgaaaaa aaatctgcat cttatctcta cgccaatcct tatgaaggag ctgtttttca |
| 38941 | ggagttcgag aaagagacac agggatgtcc agtcatcaaa gccgcagagc ctgaggaaga |
| 39001 | ataaaggatt tgtggcagga aaacccagta atgaatgtat tctgctagtt tctcagcata |
| 39061 | gaacttagaa aagaggccac agaaggaaaa gagaagtaat gttagacagc tgatgttggc |
| 39121 | aatgggcaaa gaatttcatt ctcatatgca gggcagtggc taaaggggca gtgttgtgag |
| 39181 | gaatcacatt ccaggtcatt catgtccagg gtgtggtagg aggactgtgt ttcatttatt |
| 39241 | ttgtacatgg cccagctatg accttgtgca aggaaatgct taatcatttt ctatcacagt |
| 39301 | tgcaaagaga ttcttatcct actcagaatg tacgtcttct cttctgtttc atttacagtc |
| 39361 | acaacccaag tccttgcttt gacctccaaa gcaccacttg atgtatccac ttcagaaaca |
| 39421 | cacacagaca gctcacctct ttctatccct taccttcctc ccctcttcac tcaaaccacc |
| 39481 | tacttccttt gcattcactc ttccttcagc ctgaaataat cttcctccaa atatctacct |
| 39541 | tctcactccc tcacttctct caagatacac ttaaatgtta tattccctat gaggcctccc |
| 39601 | ctggccatcc ctccccagcc ttcctatccc ccctctctgc tttattttat tctccttaat |
| 39661 | atatatcaca ctctgataaa ccattgaatg tacttatcaa gttattgcct ctctctccct |
| 39721 | ttccattgtc tccatcactg agagctctgt gaagaaaagg atttgtacct gtttcattta |
| 39781 | gtgctgtacc cccagttccc acaacagcgc aacaggcact caataaatag ttgttgaata |
| 39841 | agtgaataaa acagaagtag ctgcatattt tctggtaaca aatgatattc ttctgaaaat |
| 39901 | gtcatatttt cagacatatt tccgaaaata aattcaaatt agataaacat tgtattttta |
| 39961 | gaccatttct tctttgcatt aatcatcctt ctcaataata taacatttgt aaaacttagg |
| 40021 | ttagatatgg gctcttcaac tttccattac agaagataaa gtgaaaaggc tagacccaat |
| 40081 | ggtgttattc cttcatctac atctatcctt tggaaacaca tgaccaaatt gcttgccatc |
| 40141 | acaatctcaa aatctaccct ttggtattaa ctcacttcac ttgtccctct gtcccttttt |
| 40201 | agatggtagc catcggtctc tggagcagtg tagagtcaga acaacttcta tttggggaag |
| 40261 | aaatcattgc tggtgacctt actttcaatt actaactttc tagtgacatt tacataattt |
| 40321 | tagagaaaat taacacctac acttgtaaag ttgtggcttt cccacaccta tttatcatct |
| 40381 | ctcaatattc cttgaaaagg aaattatcaa tttatcattc tatattggca atgaaatgcc |
| 40441 | cctaatatct gtcacctata agacaattga agatgatgtg ttgaaagctt tctgaaaatg |
| 40501 | ctgatcatta ctttaaatgg aattgaaatt ccagtttatt atttccaaaa atatgatctt |
| 40561 | actgatcata ggataacatt tcataacatt tcagagattt cttccccttc gaggagccaa |
| 40621 | acccatagga cctctggact cccacagatc ctggcaggga gttcccactc ataaaagcac |
| 40681 | aggtgccctc agagtcattc agggatgaag aagcaaccct cattggccat gtcctacgtt |
| 40741 | ccccatatag taaggactgg aggagaccag tgctcaattc tgcagtctca gacagctgtc |
| 40801 | agaggagagt catgaatgtg cagtgtctag cacattacaa acgtttgtta attgactgat |
| 40861 | cattcatggt gtgacagccc tataactcag ctatcctatt cagtcagaaa ttaactcagt |
| 40921 | aatcaaagtc attaaaagga agaaaaaaaa aacctacagt accagacaga tggtggggaa |
| 40981 | atcagacaga tgaaaggaaa aatggctgta ggtcattgag taagacactg ggcagcaaaa |
| 41041 | cctgggcctt gtgcctggtt atactccaca ttatagctcc agcaaggttt ggcaggattt |
| 41101 | ccacagtcct ggcttattct aacctttctt gggagcagga gcagtgttgg tcagatagac |
| 41161 | agacacataa ggaatctgtc caactggcac cgtgtgaatt tgggctcttg gtgtacatgg |
| 41221 | ataactggga aaaagaggag agagacatgt aggactgatc ctaccgtttg tgaagtcttg |
| 41281 | ggcaagagta tgaatgaaaa cccacttctc ttcccctgcc tggctccact gcacacagta |
| 41341 | aagagcctca agcataggtg tgtggacatt gcaccatgta tccaagctct gaccatgcct |
| 41401 | cttgaaacag ctattcctca gccaccctct gaccatggga ggaatgaccc aggagaaatg |
| 41461 | accacatagg tcttgaaaat gggctcaggg ctatttacga agtcaattcc ggggtcccag |
| 41521 | gagtatggac taaaatgtga gtcaggcatg ccagatgggt atgttctatt gacttcaagg |
| 41581 | attcctcatg ctgtgggaag gaacctctcc agaagagaga cagagcagaa ccctctaaat |
| 41641 | gtggggcaca aagcaggagc ccctcttgct ggattcaaag ggtcatactg gaagagtgta |
| 41701 | ggttgagtct tattctcaca tcactcatat cacttacaca cttcttttat agccttagca |
| 41761 | gccatgccac aaagagaaac tcttcatgca tatcttttgg tccataagtc ataaatagtt |
| 41821 | atccttgatc ccatgtcttt tttagagcca tggacagaga gaagcaaaaa tataccaagt |
| 41881 | tcacactgag ttgtctccct tcatatctct tagcagtcac tgaaaggtta tgagactcag |
| 41941 | gcbgggtttc tatcctctgt ccctgaaacg acaacgttga cctcgtgatc aaccctagaa |
| 42001 | tccagagcaa gatctcagac tgtcctctct actaccagac agcacacttt gttttggggg |
| 42061 | ctgtgtgctt gaaatattag ctatggcaaa aggctttgag tcttatgaca ccccaagtaa |
| 42121 | cttttacttt aggaatttga aatacagcct tgctgtaatg ctgtctcctt aacaaagcag |
| 42181 | tacctttgaa atatttaaca acttgaaaag gaaaccgagc ttgaattttc ctttcaggtg |
| 42241 | ctcaggaaat aatgtttcac ttctgtctga aattcaccat ctcctcagac aaagaaggct |
| 42301 | cttatggtaa aaggaatggc attttctcca caattttcga ataaaagata aagagaaaac |
| 42361 | agcactgcag cctttttgtt aggatctaac aataaagaaa taatacggtt ttgccagggg |
| 42421 | agagctctgg ttttaagctc agaatacaaa aataggctga caaaatttta caaaggaata |
| 42481 | ttctcagcta ccactctgag gatggtagaa agtgaaattt caagaaaatt atattatttg |
| 42541 | attatttgat gatgattaag ctgattggcc agtcctatgt gaaattctaa agtagaagaa |
| 42601 | atgtgatgtt gtcttttctg ctcacctctc ccctcattcc tacccccaaa tctctgcctc |
| 42661 | taccccaaac ccagcctgac ctttgggaaa ggaatggggg ctgtcacttg caccgtagct |
| 42721 | cctcctgcct gcagtactct ccccccacag tccagcctcc ctctgctcag ctaacttttc |
| 42781 | ctatagctcc ttctggctac ttctaaggaa ccttccataa tgctgcccac cctctcagta |
| 42841 | acagcccctc tactatccct tgattacatg cactgtaatt ggttagtaat tgattttatg |
| 42901 | tcctctgcca aattatactc catgagagca aaaatcatga gtattatctt taatgtttaa |
| 42961 | atctccacaa ctatccccca tataagtcta gagaataaat gaatgagtga attaatgaat |
| 43021 | agaaactcaa gccattatgt tgccactcct aggatatttg gattaacttt aactgaagag |
| 43081 | taaaaagcat ttacctgtcc taaaggagac ataaaattag tgggagagta tttggagaaa |
| 43141 | aaaaagacac tgtaatacat tcttttgtgt tgcctaccct gatgtagtca ggtgtccctg |
| 43201 | atatggggtg ggctgaggat ttgaaataaa atacctaatt tgacattgta agtggaggaa |
| 43261 | tcaggatttg aaaccaaatt ggtttgacct aaatcaagct attatgataa ggacttgcaa |
| 43321 | gaaaaaagga atccataaaa accatatgaa tagctaccaa ttagtaagca tttatatgtg |
| 43381 | tcaattacaa agccaaatgc aaatcatgct ttatttatat tagccacctt ttatagatga |
| 43441 | agaaattgag acctgaatat taaaattgcc tacttttaca tagtaagtaa aggaatcagg |
| 43501 | gtttgaaccc aaattggttt cacctaaggt agaaaaccat cccagcaagt ctcctattaa |
| 43561 | ctggaaccct attgtggtgg cctgagatat aacagtagct gtggaagcgc tgtagagtcc |
| 43621 | tggccatcct atgtgctcct gatctggtcc ctcctgccac ctgcttctgc tccctgtgcc |
| 43681 | atccacccat ctggaagtct cccagtgtcc atcttcgggg gagacactca ccagagtttc |
| 43741 | cagcttccag ccagtatgga gtgcccctgt cccacagcaa tctcaccgaa atcacagcta |
| 43801 | catctgttaa aattaggcta ccaatgagtg atagatgagg gggaaaaata ataatagtgt |
| 43861 | actaaacaaa acaaatgttt atttttctca cacataaaaa tctagaggtt gaagtccagg |
| 43921 | gctggtccag aggctccaag gatctgggat ttagactccc tctttcttgt ttttccacag |
| 43981 | catatggctt ccatttctgg ggccacattg gtccaaaatg tatgcbgggg ctccagccat |
| 44041 | tgcatccata tttcagccac aggaaggagg aagtggggaa gaaaggacag gcccctaata |
| 44101 | cctgtatagt tcaagaagac tatcccgccc atacttccca accaccctta gttgaacaat |
| 44161 | gcbgtcttaa ttcaagacac tcacatgtct agccaaaaat ctgaattctg ttacaaacaa |
| 44221 | ggagaataga gatgtgcgcc acctcaatac ctcatccata gctacctttt cctttgtgca |
| 44281 | gctgtggcca agtgaaagct gaaggagctg tggtaaccct tctgaaggga ggctggggcc |
| 44341 | tttcacaaga ggctgcatga ttgacattta tcctgcatgg cctgtgaagt acagagaaat |
| 44401 | attttctctt gaagccacat catagcagtg gctgctttgt agcctgattc caccattatg |
| 44461 | cctttaaagt gcctagcaat tcagccttca catcatgcaa agaggaatat ctcccagtct |
| 44521 | ttgtaagatc agcttaattc taaccacctc cttacctccc actgcactcc tacacgcaca |
| 44581 | cacaaatctt cttcactcag agcagaacca taacccaagc cctaccacct agagactgaa |
| 44641 | gaatcaggct catgattaca aatatgcaat aattttttgt gtggataatg tcaatgggga |
| 44701 | tgatggtaag agaattcctt ggtttacaca ttgaccctct tccctgtccc ttacaatcag |
| 44761 | gaaatatttg tcccaacacc ttgtttcttc tgttgcaggc tcctgtggct gtcttgtcat |
| 44821 | gatattgttt gcctctgaag tgaaaatcca tcacctctca gaaaaaattg caaattataa |
| 44881 | agaagggact tatgtctaca aaacgcaaag tgaaaaatat accacctcat tctgggtcat |
| 44941 | tttcttttgc ttttttgttc attttctgaa tgggctccta atacgacttg ctggatttca |
| 45001 | gttccctttt gcaaaatcta aagacgcaga aacaactaat gtagctgcag atctaatgta |
| 45061 | ctgaaaggca aacctttcta taattttaca agggagtaga cttgctttgg tcacttttag |
| 45121 | atgtggttaa ttttgcatat ccttttagtc tgcatatatt aaagcatcag gacccttcgt |
| 45181 | gacaatgttt acaaattacg tactaaggat acaggctgga aagtaaggga agcagaagga |
| 45241 | aggctttgaa aagttgtttt atctggtggg aaattgcttg acccaggtag tcaaaggcag |
| 45301 | ttgactagaa tcgacaaatt gttactccat atatatatat gtgtgtgtgt gtgtgtgtgt |
| 45361 | gtgtgtgtaa gatgtcttcc tatcaaaaag atatcaaagg cacatggaat atattttaat |
| 45421 | aaaaacaaat aatatctcta atatatccac acatttgttg ccagatttca gaaaactgag |
| 45481 | ctgcaatcgc tttcctaaaa cagtagtgta ttaaatgaac atctataaaa tgtatcaaca |
| 45541 | cacattttaa aaaatttgtt taaagtatac tcttaggcca ggcgtggtga ctcacacctg |
| 45601 | taattccagc acttcaggag gccaaggtgg gaagatcatt tgagttcagg agttcgagtt |
| 45661 | acagcctggg caataaagtg agaccctgtc actaacaaaa ttaaaaaata aaataaatat |
| 45721 | aaaatatagg ctttaaaaaa gcatagtctt attaaccatg tctgttggtc aaaatctgca |
| 45781 | aactctaaaa gaagaaaaga agaaaaaacc aagcttaggg tatttttcct cccgtgcctg |
| 45841 | agtcccaatt acattcacga cagtactttc aatgaacata attgttagga ccactgagga |
| 45901 | atcatgaaaa atgatctctg cttagtacat ttgatgcaaa atgacttatt aggggctgtt |
| 45961 | tttctagcta tagtgtctcg agtactaata tgcaattatg aaaattatat taaatctggg |
| 46021 | attatgacgg tatcactgta tcatcttggt cttgttctgg ctgtcaccaa gcatgaccca |
| 46081 | ggtcaacttt ttttttcccc tgaattaccc atcaaattga tctgcagctg actaaaggcc |
| 46141 | acagctgagc ctggaactga cccttccttc atcctcaacc tgctgtcctc cagaaagcac |
| 46201 | caaggaaaaa gcagagaatg acagcaaaca gatcactagg cctctgacca caggtgctga |
| 46261 | gtactcagca gccctcatat aataggtttg aaagtactcc ttaaaataaa acactgtttc |
| 46321 | cctttggaac tatttacaag gatgaaacaa ccgtatacct gagaaataac ttgctctggt |
| 46381 | gtcaattcgc tattcgccag cagacatcag aacacaccga gtttccagat gctggttttt |
| 46441 | ccccttaaat caggaaatac acctggacaa tttctagaag actacaattc agtctagcca |
| 46501 | caaaggggat tttttttttt tggtaacagg ctagagcccg gttctgtaag tctttagctg |
| 46561 | aaatggtcca gtacaaaagc actggaaatg agtgggctag gaggacaagg accgtctcct |
| 46621 | gcgtgaggag ttggttggag gtccccaagg ccaggtaccc cctgcactct tattggattc |
| 46681 | ctctctgtct tcttggagtt ttgaaaaact ccttcgaaca ccaggctttt ttctttagaa |
| 46741 | aacaagtctc caatcgttct ctgttccgta gaaagagaaa gaaaacctgg agcagctgct |
| 46801 | gaaaaatcta atgaggaact aagaggcaaa cccacca |

A non-limiting example of a human wildtype CLRN1 genomic DNA sequence is SEQ ID NO: 47. The exons in SEQ ID NO: 47 are: nucleotide positions 1-544 (exon 1), nucleotide positions 28764-29180 (exon 2), nucleotide positions 31239-31418 (exon 3), nucleotide positions 32481-32519 (exon 4), nucleotide positions 44799-46433 (exon 5), nucleotide positions 44799-44935 (exon 6), and nucleotide positions 46128-46837 (exon 7). The introns are located between each pair of these exons in SEQ ID NO: 47, i.e., at nucleotide positions 545-28763 (intron 1), nucleotide positions 29181-31238 (intron 2), nucleotide positions 31419-32480 (intron 3), nucleotide positions 32520-44798 (intron 4), and nucleotide positions 44936-46127 (intron 7).

### Transmembrane Channel-Like 1 (TMC1)

The TMC1 gene encodes “transmembrane channel-like 1” (TMC1). TMC1 is a calcium-dependent chloride channel and interacts with transmembrane O-methyltransferase homolog in the stereocilia of hair cells. Mutations in TMC1 may lead to non-syndromic sensorineural hearing loss. For example, a D572N mutation in TMC1 has been associated with deafness (Kurima et al., Nat Genet 30:277-284, 2002, and Wei et al., J Transl Med 12:311, 2014).

The human TMC1 gene is located on chromosome 9q21. It contains 24 exons encompassing ˜314 kilobases (kb) (NCBI Accession No. NG008213.1). The full-length wildtype TMC1 protein expressed from the human TMC1 gene is 760 amino acids in length.

An exemplary human wildtype TMC1 protein is or includes the sequence of SEQ ID NO: 48. Non-limiting examples of nucleic acid encoding a wildtype TMC1 protein are or include SEQ ID NO: 49.

In some embodiments of any of the compositions described herein, the TMC1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 48.

Human TMC1 Protein (NCBI Accession No. NP_619636.2)
(SEQ ID NO: 48)
MSPKKVQIKVEEKEDETEESSSEEEEEVEDKLPRRESLRPKRKRTRDVINEDDPEPEPEDEETRKAREKER
RRRLKRGAEEEEIDEEELERLKAELDEKRQIIATVKCKPWKMEKKIEVLKEAKKFVSENEGALGKGKGKRW
FAFKMMMAKKWAKFLRDFENFKAACVPWENKIKAIESQFGSSVASYFLFLRWMYGVNMVLFILTFSLIMLP
EYLWGLPYGSLPRKTVPRAEEASAANFGVLYDFNGLAQYSVLFYGYYDNKRTIGWMNFRLPLSYFLVGIMC
IGYSFLVVLKAMTKNIGDDGGGDDNTFNFSWKVFTSWDYLIGNPETADNKFNSITMNFKEAITEEKAAQVE
ENVHLIRFLRFLANFFVFLTLGGSGYLIFWAVKRSQEFAQQDPDTLGWWEKNEMNMVMSLLGMFCPTLFDL
FAELEDYHPLIALKWLLGRIFALLLGNLYVFILALMDEINNKIEEEKLVKANITLWEANMIKAYNASFSEN
STGPPFFVHPADVPRGPCWETMVGQEFVRLTVSDVLTTYVTILIGDFLRACFVRFCNYCWCWDLEYGYPSY
TEFDISGNVLALIFNQGMIWMGSFFAPSLPGINILRLHTSMYFQCWAVMCCNVPEARVFKASRSNNFYLGM
LLLILFLSTMPVLYMIVSLPPSFDCGPFSGKNRMFEVIGETLEHDFPSWMAKILRQLSNPGLVIAVILVMV
LAIYYLNATAKGQKAANLDLKKKMKMQALENKMRNKKMAAARAAAAAGRQ
Human TMC1 cDNA
(SEQ ID NO: 49)
atgtcacccaaaaaagtacaaatcaaagtggaggaaaaagaagacgagactgaggaaagctcaagtgaaga
ggaagaggaggtggaagataagctacctcgaagagagagcttgagaccaaagaggaaacggaccagagatg
ttatcaatgaggatgacccagaacctgaaccagaggatgaagaaacaaggaaggcaagagaaaaagagagg
aggaggaggctaaagagaggagcagaagaagaagaaattgatgaagaggaattggaaagattgaaggcaga
gttagatgagaaaagacaaataattgctactgtcaaatgcaaaccatggaagatggagaagaaaattgaag
ttctcaaggaggcaaaaaaatttgtgagtgaaaatgaaggggctcttgggaaaggaaaaggaaaacggtgg
tttgcatttaagatgatgatggccaagaaatgggcaaaattcctccgtgattttgagaacttcaaagctgc
gtgtgtcccatgggaaaataaaatcaaggctattgaaagtcagtttggctcctcagtggcctcatacttcc
tcttcttgagatggatgtatggagtcaatatggttctctttatcctgacatttagcctcatcatgttgcca
gagtacctctggggtttgccatatggcagtttacctaggaaaaccgttcccagagccgaagaggcatcggc
agcaaactttggtgtgttgtacgacttcaatggtttggcacaatattccgttctcttttatggctattatg
acaataaacgaacaattggatggatgaatttcaggttgccgctctcctattttctagtggggattatgtgc
attggatacagctttctggttgtcctcaaagcaatgaccaaaaacattggtgatgatggaggtggagatga
caacactttcaatttcagctggaaggtctttaccagctgggactacctgatcggcaatcctgaaacagcag
acaacaaatttaattctatcacaatgaactttaaggaagctatcacagaagaaaaagcagcccaagtagaa
gaaaacgtccacttgatcagattcctgaggtttctggctaacttcttcgtgtttctaacacttggagggag
tggatacctcatcttttgggctgtgaagcgatcccaggaatttgcacagcaagatcctgacacccttgggt
ggtgggaaaaaaatgaaatgaacatggttatgtccctcctagggatgttctgtccaacattgtttgactta
tttgctgaattagaagactaccatcctctcatcgctttgaaatggctactgggacgcatttttgctcttct
tttaggcaatttatacgtatttattcttgcattaatggatgagattaacaacaagattgaagaggagaagc
tagtaaaggccaatattaccctttgggaagccaatatgatcaaggcctacaatgcatcattctctgaaaat
agcactggaccacccttttttgttcaccctgcagatgtacctcgaggaccttgctgggaaacaatggtggg
acaggagtttgtgaggctgacagtctctgatgttctgaccacctacgtcacaatcctcattggggactttc
taagggcatgttttgtgaggttttgcaattattgctggtgctgggacttggagtatggatatccttcatac
accgaattcgacatcagtggcaacgtcctcgctctgatcttcaaccaaggcatgatctggatgggctcctt
ctttgctcccagcctcccaggcatcaatatccttcgactccatacatccatgtacttccagtgctgggccg
ttatgtgctgcaatgttcctgaggccagggtcttcaaagcttccagatcaaataacttctacctgggcatg
ctactgctcatcctcttcctgtccacaatgcctgtcttgtacatgatcgtgtccctcccaccatcttttga
ttgtggtccattcagtggcaaaaatagaatgtttgaagtcattggagagaccctggagcacgatttcccaa
gctggatggcgaagatcttgagacagctttcaaaccctgggctggtcattgctgtcattttggtgatggtt
ttggccatctattatctcaatgctactgccaagggccagaaggcagcgaatctggatctcaaaaagaagat
gaaaatgcaagctttggagaacaaaatgcgaaacaagaaaatggcagctgcacgagcagctgcagctgctg
gtcgccagtaa

A non-limiting example of a human wildtype TMC1 genomic DNA sequence is SEQ ID NO: 50. The exons in SEQ ID NO: 50 are: nucleotide positions 1-113 (exon 1); nucleotide positions 56102-56223 (exon 2), nucleotide positions 94568-94677 (exon 3), nucleotide positions 106121-106263 (exon 4), nucleotide positions 126797-126864 (exon 5), nucleotide positions 166909-166956 (exon 6), nucleotide positions 172743-172914 (exon 7), nucleotide positions 178718-178843 (exon 8), nucleotide positions 218319-218409 (exon 9), nucleotide positions 220644-220725 (exon 10), nucleotide positions 230050-230156 (exon 11), nucleotide positions 232986-233084 (exon 12), nucleotide positions 250613-250755 (exon 13), nucleotide positions 266539-266683 (exon 14), nucleotide positions 267323-267517 (exon 15), nucleotide positions 270086-270265 (exon 16), nucleotide positions 270391-270552 (exon 17), nucleotide positions 283582-283710 (exon 18), nucleotide positions 294343-294410 (exon 19), nucleotide positions 299042-299281 (exon 20), nucleotide positions 305069-305194 (exon 21), nucleotide positions 308651-308729 (exon 22), nucleotide positions 308831-30882 (exon 23), and nucleotide positions 314151-314551 (exon 24). The introns in SEQ ID NO: 50 are: nucleotide positions 114-56101 (intron 1); nucleotide positions 56224-94567 (intron 2), nucleotide positions 94678-106120 (intron 3), nucleotide positions 106264-126796 (intron 4), nucleotide positions 126865-166908 (intron 5), nucleotide positions 166957-172742 (intron 6), nucleotide positions 172915-178717 (intron 7), nucleotide positions 178844-218318 (intron 8), nucleotide positions 218410-220643 (intron 9), nucleotide positions 220726-230049 (intron 10), nucleotide positions 230157-232985 (intron 11), nucleotide positions 233085-250612 (intron 12), nucleotide positions 250756-266538 (intron 13), nucleotide positions 266684-267322 (intron 14), nucleotide positions 267518-270085 (intron 15), nucleotide positions 270266-270390 (intron 16), nucleotide positions 270553-283581 (intron 17), nucleotide positions 283711-294342 (intron 18), nucleotide positions 294411-299041 (intron 19), nucleotide positions 299282-305068 (intron 20), nucleotide positions 305195-308650 (intron 21), nucleotide positions 308730-308830 (intron 22), and nucleotide positions 30883-314150 (intron 23).

| Human TMC1 Gene Sequence (NCBI Reference Sequence: NG_008213.1) | |
|---|---|
| (SEQ ID NO: 50) | |
| 1 | cagaaactat gagggcagaa cccagcaatc tgtgctttct ttcacaagcc ctccaggagt |
| 61 | tgctgaaatt taggaatcat tgccccaaaa agtggccctc ataatgatgc caggtaagga |
| 121 | aagacaacat tattacctat gtgtggtgtg ttaacattct ttatgaaaca tgtgtgaaga |
| 181 | cctaaatcga agactttctc catgagatat tccctaatca cattagtgaa ataaaactat |
| 241 | atttcctcta cttttctact gctacagaaa tctcccccag ttaggatagt cccagtttcc |
| 301 | tatttatctt tgtttttctt gcagtgcaca gcatgtcacc cagtaattga taagtttttt |
| 361 | tttttttgag acggagtctt gctctgtctc ccaggctgga gtgcagtggc gcgatctcgg |
| 421 | ctccccaagt agcbgggact acaggcgccc gccatcacgc ccggctaatt ttttgtattt |
| 481 | ttagtagaga cggggtttcg ccgtgttagc caggatggtc tcgatttcct gacctcgtga |
| 541 | tccgcccgcc tcagcctccc aaagtgctgg gattacaggc gtgagccacc gcacctggcc |
| 601 | taattgatat gtttaaatgt taaaattaaa tgcaatgatt tcctaaaata aacaaatgtg |
| 661 | ggacaattta ttgcactctg ttcaaattcc cattttgaag tggaattcaa acaaacatga |
| 721 | cagtaaaggg ggacagtgag cacagggaca agagaaaagg agaaagggaa gaaatacaga |
| 781 | gaactagata ggaggaagtg tcaaagactg atgggtgtaa gccaccttta cctctggata |
| 841 | ttggttttcc tttgaattgt ggtatgtggt catattgctt attagttata acatacagct |
| 901 | gtcagaagag tttttcaatt aaaatttcaa atgtaatatt aaaactgttt ggattccttc |
| 961 | ctactgggca ctggacactg tctgtggagc tgctggaaca gcgctgaata gggtcatatt |
| 1021 | ttaaaggaac tcagcctagt gcagtttctc aaccttagca ctattggcct ttggggcctg |
| 1081 | ataatccttt tttgtgaggg gctgtcctgt gcactgtagg atgtttagca gcatccttgg |
| 1141 | cttctactca gtagatgaca gtatcaaccc ccttccccag aagtgacaat taaaaatgtc |
| 1201 | tccagacttt tccaaatgtc ccctgaggag caaaattgtc tccagttgaa aaccactggg |
| 1261 | ttagtgggag aaacagataa gacagaagtt ctccatacac catgataatg ccatgatgcc |
| 1321 | atagggctgg caggagtgaa gggaagagca tctcacaaag actcgggggg atgcagggag |
| 1381 | gaggaaaaat taggaaatga agtgggctga gttgcagtta gcctgtgggg aaaatgaagg |
| 1441 | agaagggata aggaagaaga ggaggaagga gggaggaaga gtgaggagta atagggaatt |
| 1501 | atagttgacc ttgaacaact tggggtttag ggatgccgac ccttgcgtag tcaaaaatct |
| 1561 | gagcttaact tttgactacc ctcacacttt cctaacagcc tactgttgac cagatgcttt |
| 1621 | accaataaca taaacagttt attaacacat attttgtatg ttttatgtat tatacactgt |
| 1681 | attcttacaa taaggtaagc tagagaaaag aaaatgctat taagaaaatt gtaaggaaga |
| 1741 | aaaaatatat ttactgttca ttaaggggaa gtagatcatc ataaagctct tcatccttgt |
| 1801 | catcttcaag ttgaataggc tgaaggagga gggggagaga gaagagactg gccttgctgt |
| 1861 | cttaggggtg gcagggatgg tagaggtgca ggaggtggaa ggggaggcag gaaaggcagg |
| 1921 | cacactcggg gtagctttcg ttgaaagaaa tcttcatatg agtggaccca tgcagttcaa |
| 1981 | acctgacttg ttcagagtca actgtacttg ttccaggcta agaacagaga ataaaaaaaa |
| 2041 | aatgtataga ggcctgaaac agcatattat attaaaagaa ctcaaagccg gtaaatgctt |
| 2101 | tgctactgaa gcataaagtg agaaaggggc atcatgggct ctgaaattgc agccacaagt |
| 2161 | ctacagaaca catttttcat agcccaaggt cagtcttcta ttaccctttg cagtgaggtt |
| 2221 | atcatcatcc attttcactc gtcagtgctg gggagcccat taaatacttg ccactatact |
| 2281 | tggtcccaac agttaataga ctgtagcatc ttttatttct tcacctagcc tgaaatcagt |
| 2341 | tgagcttttg cagaatctta gatatgtcgt ttttttcttc aggtttagaa tggggcactt |
| 2401 | aaaaagatgc atagtacttg aaaatagaga aagaaaatag catgtagaaa tttgtgcctt |
| 2461 | tattctgccc aaacgtctca aaaaggaaaa aaataatttt aagggtaaat attgtcatca |
| 2521 | atttatctga gctcttttga cttcctttga gcctgaaaaa caggatcagt gtctttttag |
| 2581 | tatttaccat agtgcctgat atgtagaagg tgtttgacca atgtttatta gataaatgtt |
| 2641 | cacttttatt actagtaata ttaaccacat tttaatagag actaataata catgataaac |
| 2701 | ttctcaagga tgcgatctca tttgatcact ataacaaccc aatgaggtaa gcgaggaata |
| 2761 | gttgttaact ctaatacatg ttatatctta tccttgaccc agtattaccc cagtcatgcc |
| 2821 | ttcctttccc tgcaatgaaa gaaaaccgat aaacacaaaa tgttgtatcc catataagca |
| 2881 | ttcattctta tggtagataa gtaaatggaa aagataggta tgcattctca atttttttaa |
| 2941 | gtcctagaat tataaaacag taaagaagtg agaagagaga gaaatcttaa gggaccaacc |
| 3001 | tcttgatttt acaggtgaga aaactgaggc cacatagcca tttagtgcag agccaggatt |
| 3061 | ataaaccaga tgtttggaat cctatgtggt ggtggacacg aggatatact aattagcgtc |
| 3121 | gttaccggaa aattccacat atctctaagg agcagatcac cacacacctg cacagcccaa |
| 3181 | gcctggattg tggaggtttt tcttgtagag tttgtctttg atagaaagat acggtccaga |
| 3241 | aggccggctt caaggacata agaaagggca tattcaaaac aaatacatgt atgcctattc |
| 3301 | ttttaaaccg gtacttaaca atagtatcca ggtgtccaaa tataatagct tatctgtttt |
| 3361 | attttttcca gaaaaggaaa caaggtctta gagaggttac attttgcata ggattttgtt |
| 3421 | agccaagtca gcatctgttt gcatcttaaa aatgagtcat acctattcct tgtaagtagg |
| 3481 | acaggagggc agctggaatt tcccagctca ctccttgtca ctgcttcctg cctctgctgc |
| 3541 | tctttgtcct ccacagcacg cttcattggc taaaaagtga accagatact acattacctc |
| 3601 | agggcagttg taagtactga gtcagatgcc ttaggcttgt gtgatgggaa gtttagggcg |
| 3661 | agaccaatgt cttgctttaa caacagtgtg aataagttaa tttcttttga gattttatta |
| 3721 | ctcaatattc cacccagaaa gaaacacaga tgacactcag ccaatcaaag gccatctggg |
| 3781 | actgacaaac gcagagcttg ttgtagatga caaggctcag tggatggatc atcactgaaa |
| 3841 | gaaagggaaa cagtttgaac tgttgtatct ctctccttgc tcacatgaaa aacaaaaaaa |
| 3901 | tccttggcca gttgcggtgg ctcacgcctg taatcccagc actttgggag gcccaggcgg |
| 3961 | gcagatcact tgaggtcaga agttctggac cagcctggcc aacatggcaa aaccccgtct |
| 4021 | ctactaaaaa tacaaaaatt agccgggcgt ggtggcacac acctgtattc ccagctactt |
| 4081 | gggaggctga ggcatgagaa ttgcttgaac ctgggaggtg gaggttgcag tgagccaagg |
| 4141 | tcgtgccact gcactccagc ctggatgaca gagtgagact ccgtctcaaa aaaaaaaaaa |
| 4201 | aattccttaa ccacagacta agtaagcatt attgcacaga gtgtcattcg ctaatcttgc |
| 4261 | ttgtgcctga actttgaaag caccagcccc ttgctttggg ggcbgggggc gtggggtgta |
| 4321 | agtgtgtgtg tcctctagaa gaagacaaca ctgagaacag aaggagagga tcttggagat |
| 4381 | ttgttaattt cctattgcag ttagccttgt ctggggctct cactttagca cttaaaataa |
| 4441 | catactcctt tgcacagatg ttattttttt ctcttcatgt gtacaacttg aattatccaa |
| 4501 | tggccttgct ttgaggatat tcatcctatc ttattgatcc tgtcactgtg taggacaatg |
| 4561 | gagaacacag taaaaaaccc tcaatggata ctacttacac taaattaact ctatgaattc |
| 4621 | aaaatttgaa gatgctactt aatcatgtaa gcctggttct ccaaaacttc atctgccatg |
| 4681 | ggaaaaatca aagcttgtaa aatgattaaa cttgtaaagg aattaaactc taggattcac |
| 4741 | tcattttata attggtattt tcaccctaag agctttcttt ttcctctctg gtgtttaaaa |
| 4801 | acaaagtaaa accagacaaa acaagattaa ctaaattaaa acccctgtga ctttaaaaaa |
| 4861 | tgtagccaag aataatgtgc aagtgtctag ttaggccaat agatatcctt aacatacgtg |
| 4921 | agcbgggaac acaaatgagc atgaagggca aggaagagca ccaccccctg cttcttttcc |
| 4981 | tgctcctgtt cttgcagagg gtcaggcttg gagaccacat atggtcagct caacactcaa |
| 5041 | gtccaccaag cagcccatga ggaactcttc cttccagagt tgtgcacacc atgcagtgtg |
| 5101 | gtccacctgc agaagcacac aacaggcgct gcctgggcca gaggtccatt caggccatgg |
| 5161 | aattggtctc aatgccattt aggcatgaat ccagggtcct gggtaccaga cgcatggtct |
| 5221 | aggtaggggg tgtcatgtac taggtgagca tggggttccg ctgggcatgt tcccttgtct |
| 5281 | tgtaggatgt tagctctgag gggcgtggct ggaagaaggc tagccttgag ccctctaaaa |
| 5341 | ggcagggctc agggcaaagc tcccagttgc tcaggcctgt gggtaatact gcttaaaaat |
| 5401 | ccccttgaga acttctttaa aatgcaaata tctcgattct tttgtagttc aaaagcatga |
| 5461 | tgattgggtg ttcacaggca tgtgtaagac gtgacaccct ctgaacctcc ttatgacccg |
| 5521 | gtacattacc tgtctgacct gaaaaaaaaa tgcaaatttc tggattctac ccgcctcccc |
| 5581 | gcactccgac attacgatgt agcagctctg tggtaggatc caggaatctg cctgtgtaat |
| 5641 | cacaggttga tattctggtg tttcctggtt ttgctctgct gatttggggg atgtttacca |
| 5701 | gcaggagagc agcctctttc ctctgccctc ctcccttgct gccctcccca gaccccagtg |
| 5761 | actcacgctc ctcctgctcc tcctgttgct ggcctctctg ttgtggttgc cagcgctact |
| 5821 | ttgcagacgc tgcctgctcc ctgcacgcca acacagattc tgtttccctg cgaccgctgc |
| 5881 | tgctgttgtt tgttccgtcc ctggtttatt gtaggagagc tgattgttct gttgccactg |
| 5941 | ctaccaagct gcaaacacca ggctcacggt gcaaggggaa ggggcttctc ctctgggtgt |
| 6001 | tctgcctcta tcgcctaggc tctcaggctt tgttgccaat ggcactgcga gaccctaaac |
| 6061 | tatggccttg tgattcccac atctccattt agttctttct tttgttctct aagatctgag |
| 6121 | aaggagccca tgtctttcag tgttcctgtt ttcttgcgat acttcccccc tccccgactc |
| 6181 | cagaatttag ggcaaccttc tgcbgtggtc tgaatgttta tttctctcca ccccgccaaa |
| 6241 | cttatatgtt gaaatcttaa cccctaaggt ggtggtatta gaaactgaag cctctgggag |
| 6301 | gtaattaagt tataaggaca aagccctcat gaatgggatt actgttctta taaaaaaaag |
| 6361 | gccccagaga gctagctagc tccttccacc atgtgaggac acaacgtgaa ggtgctgtct |
| 6421 | atgaggaagg gtccctcacc agacaccaat catctggcac attgatcttg aacttcctat |
| 6481 | cctccagaac tgtaaacaaa taaatttctc ttgcttataa accataagtt catgattttt |
| 6541 | tttttttttt tgagacggag tttcgctctt gttgcccagg ctggagtgca atggtgtgat |
| 6601 | ctcggctcac aacaaactcc gcctcccggg ttcaagcgat tctcctgcct cagcttccca |
| 6661 | agtagcbggg attataggca tgcgccacca tgcctggcga attttgtatt tttggtagag |
| 6721 | acagggtttc tccatgttgg tcaggctggt ctcgaactcc tgacctcagg tgatccaccc |
| 6781 | gcctcggcct cccaaagtgc tagggttaca gatgtgagcc actgcacctg gccaagttca |
| 6841 | tgatttttgt aattagcatc caaaatggac taaaaccccc atcctcctga aggttctgaa |
| 6901 | tctgaggacc caagacttgg ctctcagcct taatgtcctg ctcccactat tttcaggggg |
| 6961 | cccaatataa atttcctgag tttttgaggt ggttgggctt gcagactgac ctttagtatg |
| 7021 | ccctttgtta catttacaaa atagattgct tacagggtta gacaaataca gttgaccctc |
| 7081 | tgtatccctg ccttccatat ccatgggtca aaattatttg gaaaaaaaaa tgtgtctgta |
| 7141 | ttgaacatgt atagattttt tttttgtata tttcctaaac tatatcgtat aataactact |
| 7201 | tacagagcat tcatattgta ttaggcatta taagtagtgt agttatgatt taaagtatgt |
| 7261 | gagagaatgt gcacaggtta tacagaaata ctacaccatt tttttttttt taatcaggga |
| 7321 | ctgaagcatc ttttcagcta tgactttggt atccggtggc gggtggggcg gtgggtgggt |
| 7381 | cctggaacca ttcctccatg aataccaagg gataactcta attgctaaat cgtttcctat |
| 7441 | ttttgtgaac tggtaaagga ctgcatgaga aaggccaaaa attaaagaat aatatgctta |
| 7501 | ttcaagcccg gtgtggtggc gggcacttgt agtcctagct acttggaagg ccgagatggg |
| 7561 | cccaggagtt tgaggctata gagggctatg gttgcacctg tgaatagtca ctgcacacta |
| 7621 | gctcgggcaa catagctaga ccctgtctct aaaaaagaaa aattctgctg taataaacct |
| 7681 | gcacgcataa aatgatgtct atgtgtctat atagtgtgta tatacatatg tgtttaggaa |
| 7741 | atgtgtatat atgtgtgtgt gtttgtgtgt gtggatactg tattagttaa gacaaaaaaa |
| 7801 | tctggcttca tatgttataa aataatgtct ggagctatcc actcaagtgt gctttcttgt |
| 7861 | ttctctgttt tttttttttt cctatttcat tcagtggttt tcactgcctt tgggaaaaag |
| 7921 | tgacacagca aaacacttag aagaatcaaa tgtttatcct ttgagaatgt ggtctgtaca |
| 7981 | taagagaaaa catttagaat caaattatga gactaaacca gtttccctca cctgcagcta |
| 8041 | gataaaaatt tctaagagga aggagagaat caggaaagat tgaagacttg actccctgat |
| 8101 | aagggttctg cccgagttct gagaatggag aataaaagac accctccccc tctctaatag |
| 8161 | attttgtcag tgaaggtcag agaggcccac gaaatcaggt ctggggagca aggtaaaacc |
| 8221 | cagaatttct acatgatcat gttaaagagg gaataaagga atatcattat agtgtatctg |
| 8281 | ggcaggtggg catgagacag cctgattgag ttcctgaaat cccaggtcgg tgtgggagca |
| 8341 | gcagaatatt ttaggtggct gagagctcca tagtttcttt ctttctttct ttctttgaga |
| 8401 | tggagtctca ctctgttgcc caggctggag tgtagtgacg cgatctctac tcactgcaac |
| 8461 | ctgtctccct ggttcaagta attatgtctc agcttcctga gtagcbggga ttacagatgt |
| 8521 | gtgccaccac acctggctaa ttttttttta tttttagtag agatgggatt ttatcatgtt |
| 8581 | gcccaggctg gtctcaaact cctgacctca agtgatccac ctgcctcagc ctcacaaagt |
| 8641 | gcbgggatta caggtgtgag ccaccagcag tcccagctac tcaggaggct gaggcagaag |
| 8701 | aatcgcttga acccgggagg tggaggttgc agtggaccga gattgtgcca ctgcactcca |
| 8761 | gcctggtgac agagcaagac tccatcttaa aaaaaaaaaa accttctcaa ggtattttct |
| 8821 | gtgcttcttg ttttctcttt tgtatttttc atctttatat ctttgtgttt cattctggat |
| 8881 | aacttttagt gacctgtctc ctagttcact aattctcttt ttagctatgt ctagtattat |
| 8941 | aatttgttta ttgaattatt aagtttaggt atcaaatttt cagctttagg atatctaatt |
| 9001 | tgccacttgt tttctgccat gatttttttt ttttttttga ggtggagtct tactctgtca |
| 9061 | cccaggctgg agtgcactgg tgtgatcttg actcactgca agatctgcat cccgagttca |
| 9121 | catcattctc ctgcctcagc ctctcaagta gcbgggacta caggcgcctg ccaccacgcc |
| 9181 | cagctaatta ttattattat tattattatt tgtattttta gtagagacag ggtttcacca |
| 9241 | tgttagccag gatggtctcc atctcctgac cccatgatcc acccaccttg gccttccaaa |
| 9301 | gtgctgggat tactggcatg agccactgca cctggcttct gccataattt ttaaccttga |
| 9361 | gttttaccta cttaaacata gcaaacacag ttgtttatat atttatctaa taattccagt |
| 9421 | atcaggagcc tctggagtct acttttgtct actttcattg ttttttgttt tttaaattta |
| 9481 | cattaacagt gtctagtctg tatttatgcc tggttacttt tgattgtgtg ctagacattg |
| 9541 | tttttggcaa ataatttttt aaacacaata tgtacaaatc atatgaagcc tagactggtg |
| 9601 | ttttgttcac caaaaaagat tttaaattac ttcagttacg ttcctaggga cactattaac |
| 9661 | tcaggatgat gttaattgaa gtttcagatt tgatattttc caggtcaact ggaccaactg |
| 9721 | tccctgctat atgatttatt tcctaaagca atatcaagtt cagctctgca cctctcttat |
| 9781 | atactatgga gctctcagtc acataaaata ttctgctgct ctcacactgc cctgtagttc |
| 9841 | ataagaggaa cagtttactg ttaatccgta ttactcttac atctagggtg tagctctttg |
| 9901 | agctaccagt ccaaagtgaa gaataattta ctgaattcct actttgtctg gctctaggca |
| 9961 | ctgatttttt ttaccctctt ggccacctat cttcaattgg aaaatgccct caattcaaaa |
| 10021 | atgctccaag tgccaagctt tcctatctga attcccattt tttcctgtat ctggctggca |
| 10081 | attcattaga atcctgctag ccctctgatg cttctaagat tttcatcttt tatttttatt |
| 10141 | tatttattcg agatggagtc ttgctctgct gcccaggctg gagtgcagtg gtgtgatctt |
| 10201 | ggctcactgc aacctccatc tcctagggtt aagtgattct cctgcctcag cctcccaagc |
| 10261 | agcbgggact acaggcatcc accagcatgc ccggctaatt ttgtattttt agtagagatg |
| 10321 | gagtttcacc atgttggcca ggctgatctt gaactcctaa cctcaggtta tctgcctgcc |
| 10381 | ttggccttcc aaagtgttgg gtttataggc attagccacc atacccagcc tgaagttttg |
| 10441 | attaaaaatg ctttactggg ctgggcatgg cagctcacgc ctgtaatccc agcactttgg |
| 10501 | gaggttgagg cgggcagatc acttgaggtc aggagttcga gaccagcctg accaacatgg |
| 10561 | tgaaacccca tctctactaa aaatacaaaa attagccagg tatggtggtg catgcctgca |
| 10621 | atcccagcta ctcaggaggc tgaggcagga gaatcgcttg aatctgggag gcagaggttg |
| 10681 | cagtgagccg agatcgtgcc actgtactcc agcctggaca acaagagcga gactccgtct |
| 10741 | caaaaaaaaa aaaaaaaaaa aaaaaaaaaa aaaaaaaaaa aaaaaaactt taccggaatt |
| 10801 | aaagcccagt catcttgaag ccatgatcat gacgcbgtgg aagaaccggt gaggtaggat |
| 10861 | gagacatatt ctcagatgct atgctgattt ggggcbgtga tgattttgga ccatagacta |
| 10921 | tgatgaggct tcaaaaagag tgcaatagtg tattttaatt tctttggtta gattcaaatt |
| 10981 | aatcttcaga aagacaaaat gcagatgtag tcttgttaga gcttgctgat gagactaaat |
| 11041 | tttccaagtg tttgaggaat gcctaacagg ggaaaggcaa agcaaactat ttttaatgac |
| 11101 | cttatggctc tgaattgcta aaagggagtc agtccaccag gctcaggcta cttggagtag |
| 11161 | aggatcctgt ttcagtgaca aactggctca tcccttaatc tgccataaaa tgatggctta |
| 11221 | gcatacatgt aggagcbggg gctaagggag aaggaggtgc tgtgttttgc agaagaagct |
| 11281 | acattctgta gtaaggcctg actcttccct ggccttggag gggaaatact tgtgagacag |
| 11341 | tggccaggat cagctgaagt gccctctctt cttgctttgc ctatgggaca ctgagcccct |
| 11401 | ttttgtggaa tatattcatc cagagaaccc tcacacctta taatgcataa agtgataaag |
| 11461 | gcttggagat ctggagaaac ccaaggtgcg atccctggct ctagaagaaa ctctgggcag |
| 11521 | ccagcatgat ttacttttct ctgctccaaa gactggagga taaattaaag gaaaagaggg |
| 11581 | tatgacccag caatccactg gtgcgagttg ttgctttaag caaagactac tgcttctctt |
| 11641 | cctgattttc ccctgaccat actctcccag aggtgtctcc cagcaaccac cagagttgtg |
| 11701 | gttaagtaca cctagccaat ccaataaatc taaaggagat gcagtccaca caaccttgca |
| 11761 | tgacttgtca tgtgagttac tgttctggag agacaaactc aggctggaaa cttatttgag |
| 11821 | atagcttatg aaaacaaaga taatttatga taaggataca ggcactctca tacaatctaa |
| 11881 | ggtagaaaaa cagctaagca tcagaacaaa ctggaccaaa gacttaaaca cttctgctcc |
| 11941 | tttgtgtaat tggtttcatg cgtttctctc tctcccttcc tctctctctt ttctctctgt |
| 12001 | ggactaggtt tttctacttt tcatatacaa attttaaaaa tacaatttct ggccaggtgc |
| 12061 | agtgactcac gcttgtaatc ccagcacttt gggaggccga gggaggtgga tcacttgagg |
| 12121 | ccaggacttt aagaccagcc tggccatcat ggtgaaactc catctctatt gcaaatacaa |
| 12181 | aaattagccg ggtgtgatgg tgcacgcctg taatctcagc tacttgggag gctgaggcag |
| 12241 | gagaatcgct taaacctggg aggcagaggt tgcagtgagc tgagattgtg tcactgcact |
| 12301 | ccagcctggg taacagagtg agaatctgtc tcgaaataaa tgaataaata agtaaaataa |
| 12361 | acaataaaaa taaataaaga caattttaaa aatttcccaa cattaagtgt cagcaatttg |
| 12421 | gagagggaca gctgtctctt gttctctctc ttctccaatt ctcaattcct aggtctgatg |
| 12481 | ctttccctgg ctcaactgac tatggttgtg gggtagggcc atgaaacaga aatatcacat |
| 12541 | taggatccaa cctttaatta tgtagatgga cagcagttta cagaaaaagt ggtggggagg |
| 12601 | tggagggagg tgctgaacag acattctaaa agggctccac caaagaaagc tatttgttat |
| 12661 | atttatgaac attctggaaa attctttcaa ttgtgttcat gaataaatta tgattggtta |
| 12721 | aaagctaaca cacatgtaga ttatataact tctaaaattt gtatttggta tctgaatcta |
| 12781 | tatattattt tggcatgtat agaaaaatcc cagatatttc aaaggctatc attagaaaaa |
| 12841 | cgttaatgaa aaggttaatg gaattctata tatttagttc ctgtgagaat gtccagttta |
| 12901 | actttcttaa tcaactgatg aagaaacaga actgaaatca caagattata aagatatttt |
| 12961 | aacgatgcat ttaggttttc attctaaatg ctgaaaaaag gtatccctgc aaatactggt |
| 13021 | taagaaatta agaagtccaa aacccaataa tgtagttggc agcctgttat aaagatttag |
| 13081 | agtattcaaa gataaaggca aataaaaaga ctacttttta taccagagta gaaacagttt |
| 13141 | attttcatga tcactactct tacttcaaat atatctatta tctaagttga tgcttgcaaa |
| 13201 | aagtcctgat tcttacacaa aggcagtcga ttcatggatc tcttagagag agagaaaaaa |
| 13261 | aaaaagctag ttgggtgtgt tacagttata tgaatcatgt aggatgctgc ttaacgctat |
| 13321 | atttatcatg gatgaaggca tgttttcagg ggaaactatg agtctaccta aagttaccca |
| 13381 | gttcacctct gttccaaggg actcatcttt tcagcatttt gtttagcatg ttacaaagta |
| 13441 | ccccgatgtc ttctaaacta ttaggattgc aggaggaaac tctcccagag ctgctagaag |
| 13501 | tcatccttga aagtttggat ttctcccgaa aacttgtatt tctatcattg ttttaactat |
| 13561 | cttcttgaat cctcttgatc ctgacctaca tgtcctcttc ccttgattgc atttaaattt |
| 13621 | agctgcatat aaccttgctc ttttccttct aatccaacct ataaaccata tcaggggagg |
| 13681 | atatgtgatt tgtcaatttt gttgcctcac ttcctcctca agaactttca gtggctccat |
| 13741 | tttacctctg agattaaatt gcagccactc atcctgagtt ttggtgttcc acaatctgac |
| 13801 | atatctagtc agctttgtgt tctagtgtgt ttgtgttgct ataaaggaag actgagacta |
| 13861 | gatagtttaa taagaaaaga ggttgatttg gctcacaatt ctgcttgctg gaagattggg |
| 13921 | catttggtaa aagccttagg ctgcttctat tcatggcaga agagtaagcg gagccttcat |
| 13981 | gtgtggaaat catagggcaa gggaggcagc aagggaggtg tggagaggtg ccaggctctt |
| 14041 | tttaacaacc acttctcatg ggaactaata cagtgagaac tcactgactc tttctacagg |
| 14101 | gagggcatta atttattcgt gaatgattct cccccatgac caaaatacct ctcattaggc |
| 14161 | cccacctcca acattgggat aaaatttcaa catgaggttt gaagggggga caaacatcca |
| 14221 | aactatagca ttctgctttg gccccctaaa acttatgtcc ttctcacatt caaatacaat |
| 14281 | catttcttcc taatagtccc taaaagtctt aacttgttcc agcataaact caaaagtcca |
| 14341 | aagtccaaag tatcacctgc gactcaaggc aacttcttta cagctgtgag cttgcaaaaa |
| 14401 | taaaaaatga attatattct tccatgatac aatggttgta caggcattga gtaaacattc |
| 14461 | ccatttccct aagggagaaa ctggccaaaa gaaagtggta acaggtccca tccaagtctg |
| 14521 | aaacccagtg cggcagacat tctttttttt gaggcagagt ctcgctccgt cacccaggct |
| 14581 | ggagtgcagt ggtgcgatct cggctcactg caagctctgc ctcctgggat cacgccattc |
| 14641 | tcctgcctca gcctcctgag tagcbgggac tacaggcagc tgccaccatg cctggctaat |
| 14701 | tttttgtatt tttagtagag acggggtttc actgtgttag ccaggatggt cttgatctcc |
| 14761 | tgacctcgtg atccgcctgc ctcagcctcc caaagtgctg ggattacaga cgtgagccac |
| 14821 | cgcgcctggc cagacattaa ttcttaaaac tccaaaataa tttcctttga ttccatatac |
| 14881 | cacatcctgg gcatagtagt gtgaggggtg ggtcctaagg ccttaggcag ccccactctt |
| 14941 | tggctttgct gggtgcagcc cacgtgactg ctctcacaga ttgaagccca atgcctgtgc |
| 15001 | ctttttcagg ctgcagttgt atgttgctgg tggctctata attctggggt ctggagggtg |
| 15061 | gtagccctgc tcccacacct ccaatagata atgcccaagt agaggctgtg tgtggttgat |
| 15121 | ctgaccctat ggcagacttc tgcctgcctt cccaggtgtt ccaataaatc ctctgaaatc |
| 15181 | taggtggaag ctgccaagcc tccaacattc ttgcattatg ggcacctgca gatttcacac |
| 15241 | catgaagcca ccaaggctta ccacttacac ccacctaagc agtggctcaa acagtacctg |
| 15301 | gggccattgg agccatggat ggagccagag tggccaggat gcagacagca gcatcctgag |
| 15361 | gcagcacagg gcagtggtac cccaggcttg tcctgcaaaa ttattttgtc ctcctaggcc |
| 15421 | tctaggcttg tgatgggagg agcagcctca aaggtttctg aaatgccttt ggggcctttt |
| 15481 | ttttccaaag tcttggctat tagaacctag ctcccactta gtcatgctaa tatctctcta |
| 15541 | gcaagtgttt ggctgcatcc ttgaattcct ctcctgaaaa cactctttct tcatctacca |
| 15601 | cagggccagg ctgtaaatct tccaaatttt tatgctttac ttggttttta gttacaattt |
| 15661 | acaactttag gtcactcatt tgctgccata tctagtttac aatcatattt acaattaaaa |
| 15721 | gcagtgtaaa ccaaaaagta tctgagacag gtctcaatca atgtaagggt ttattttgcg |
| 15781 | atggttaagg accatggccc atgacacagc ctcaggaagt cctgagaaca tgtgcccaag |
| 15841 | gtagttttgt gtagcttggt tttatacatt ttagggagac agaagttaca gacaaaaaca |
| 15901 | taaatcaata catgcaaggt atacattggt ttggcccaga aaggcaagac atctacaagt |
| 15961 | gggggcttcc aggtcatagg tatattcaaa aatttcctga ttggcaatgg ttgaatgagt |
| 16021 | taagctctgc ctgaacagtt gaagtcagct tgagttaaga taaagaggtt ggctgggcac |
| 16081 | tgtggctcat gcctgtaatc ctagcacttt taggaggctg aggtggtcag attgccacct |
| 16141 | gggcaatctg tttgagatca gcctgggcaa catgatgaga ccctgtcttt ccaaaaaata |
| 16201 | aaaaaattag atgggcatgg tggagcacac ctgtagtcct tgctactcga gagactgggg |
| 16261 | caggaggatt acttgagccc aggaggcaga ggttgcagtg agctgagatt gcaccattgc |
| 16321 | attccagcct gggtgacaga gccagaccct gtctgaaaaa aaaaaaaaaa aaagtgaaag |
| 16381 | tcaaggttct tgtcatgtgg atgaagcctc caggtagcag gcttcagagt gaatagatga |
| 16441 | tgaatgtctc ttattagaac ttaaaaggtg tcagactctc tggaaaaaac ttggtaaggg |
| 16501 | agagattctc tacagaacac taacttcctc cacaagagac aacattgcag ggccttttca |
| 16561 | aaatgtcaaa taaatatatt ttgggataaa atacttgtat tgtattgtat tgtattgtat |
| 16621 | tgtattgtat tgtattgtat tgtatcgtat ttttgagaca gagccttgct ctgtcatcca |
| 16681 | ggctggagtg cagtggcacg atctcggctc actgtaacct ccgtctcccc agctcaagcg |
| 16741 | attctcctgc atcagcctcc tatgtagctg ggattacagg tgtccaccac catgcctggc |
| 16801 | taattttttt gtatttttag tagagatggg gtttcaccat gttggccagg ctggtcttga |
| 16861 | actcttgacc tcaagtgatc tgcctgcctt ggcctcccaa agtgctggga ttactgggat |
| 16921 | tacaggcatg agccaccaca cctggacaat aaaatacttt tatttccttt aggtcttgct |
| 16981 | atgtcatgtg atgctatatc agagtcgggt tcaaatttgg tatcttattg ctacaaagag |
| 17041 | tctgtcttgt cagtcatatg atctctgttt taatgttaat gctggtcatt tgtgtctaaa |
| 17101 | ctccaaaggg ggagggtgta ataaggcatg ttgggtaccc tctcatcttg ctatggcatg |
| 17161 | agctagtttt tcaggtttct ttgggatctc cttgaccaag gggtccattc agttgattgg |
| 17221 | gggagtttga attttaattt tgatttccag cagccacgcc acttcttgaa tgctgtgttg |
| 17281 | cttagaaatt tcttctgctg ctgggcatgg tgactcatgc ctgtgagagg caggcacttt |
| 17341 | ggaaggctga ggtgggcaga tcaattgggc tcaggagttt gagaccagcc tgggcaacat |
| 17401 | ggcaaaaccc catctctaca aaaaatacaa aaaaaaaaca acaacaaaaa aacaaatcag |
| 17461 | ccaggtgtgg tggcatgtgc ttgtagtccc agttactcgg gaggctgagg tgggagaatg |
| 17521 | acctcagcct aggagatgga ggtttcaatg atcctagatc gtgccactgc attccagcct |
| 17581 | gagtgacaga gcaagatcct gtctcaaaaa caaaacaaaa caaaacaaaa tttccgccag |
| 17641 | atatcctagg tcatcactct taactgcagc cttccacaaa tccatagggc atggacacaa |
| 17701 | cacagccaag tttttttttc taaagcataa catggaaccc tttgctctag ttcccagtaa |
| 17761 | gtttctcatt tccttctgag acctgctcag ccttgccttt actatctata tttctatcag |
| 17821 | cattttggtc aaaaccactt aactaatctc taagaagttc caaactttcc ctcatctgcc |
| 17881 | tgtcttcttc tgagccctcc aggttcttcc aacctctgtc caatgccctg ttccaaagtt |
| 17941 | gcttccacat tttcagttgt ctttacagca acatttcact acttggtacc agtattctga |
| 18001 | ctttgtccat tttgcattgc tttaaacgaa taccggagga tgggtcattt acaaagaaag |
| 18061 | gggtttattt ggctcacagt tctgcaagcc tgtacaagaa gcatggtgcc aggatttgct |
| 18121 | tttggtgaag gcaaggaagc ttccattcat ggcagaaagt gaaggggagc ctccatgtgc |
| 18181 | agagatcaca tggcgagaga ggaagagcgt gagaggaggg gaaggtacca tggtcttttt |
| 18241 | aacagaggta caggcttttg agggaactaa cagattgaga actcgctcac tcccacccct |
| 18301 | gccagggagg tcattaatct attcatgagg gatccaccca cataacacac acatctcact |
| 18361 | aggccccacc tccaacattg ggatcaaatt tcaacatgaa gtttgggagg gcgaacatcc |
| 18421 | aaagtacagc accttgtttc ttgttgtttt ctaatatttg ttctttatcc caggaaatgg |
| 18481 | ggggtaggaa ggtgagttct tggtgtactc taagcagtca tgctcagcct gtactatcaa |
| 18541 | attttattcc atttattctt ctgttgaggc ttgcatgact tccctaaggt acaatggcag |
| 18601 | agatagccac atgggtagta aaatcagggg cagacagacc agctttctgc caggaattta |
| 18661 | cctcagggaa attcctcatc tttgactttc ctcgttctga cttttttttc ttttaattcc |
| 18721 | acctcctctc caaacctggc actattcctt tccgatcttt atccttgtcc ttactgtttt |
| 18781 | gaagttggct cttatgcctg tttgtctttg caatctgtct ctgcgtcgcg tgttggtagt |
| 18841 | catttatttt tgtccctagt catgattccc agaaccttgc tgtggtcatt ttgatgcccc |
| 18901 | accctttgac tattacaaac caggagagct catgcaagtg gccacatgct gtagtggtta |
| 18961 | agagcacagg ccccgtagct gaacttgaaa ctcagcatga caacatgctg gctgtgtgac |
| 19021 | taaggcaaat taaatgacca ctctgtgtcc ttattttctt atacataaaa tggagctaca |
| 19081 | atagtacctc ctcataggga tgttgtgaag cttcagtgag ttaatacaga tataatgatt |
| 19141 | agttatctgc ctccattaaa tccaatcatc aatactaccc agctttatta aaacttctga |
| 19201 | cttttgtttt ggatgtggcc aggtcctgaa ctgaattgtt ttttccaggt caaatttaaa |
| 19261 | tctaccctcc tttccttttt ctcaatgcca aattgttctt cagtttccat ctctagcccc |
| 19321 | ataatctttg atttctcttg cccacatgga tggttacctt ttctgaaatg tcatagctct |
| 19381 | tacagtgcta gccacctatt ttgatgccaa atctttacac caaccagtgt tatcacttaa |
| 19441 | tcaattctta catctatgtc ttaacttctc caaccaaact taagacttgt tggtgccaag |
| 19501 | aatcaaatct catacttttg agttattcaa accatacaga acagtagata ctgaataagt |
| 19561 | atttgtctga atgaattgtt tgaataactt aaagagccta gaagcggctg ggtgcgatgg |
| 19621 | ctcatgccta taatccgagc actttgggag gctgaggcgg gccgatcacc tgaggtcagg |
| 19681 | agttcgagac cagcctgaca aatatgatga aaccccgtct gtactaaaaa tacaaaaatt |
| 19741 | agccgggcat ggtggcatgc acctgtaatc ccagctactc aggaggctga gacaggagaa |
| 19801 | tctcttgaac ccaggaggtg gaggttgcag tgagccaaga tcacaccatt gcactccagc |
| 19861 | ctgggcaata aaagcaaaac tccatctcaa aaaaaaaaaa agagcctaga agctttccag |
| 19921 | ggtggtcttg ggggtattgg ctttagaaga ggaagcttga tagatgaaaa tttccagatc |
| 19981 | aacggcattt tatgtacttc tcagaaacaa ggtatgacta gactgaaaga tctgaagtca |
| 20041 | ggaagaaact tcttggtatc agtctgtgcc cttaggaaga agtgaggtca taagctctat |
| 20101 | tagcacacat agctgaaaaa ctgaaagagg atgtaagaga aaggattgtt aaaaaacaaa |
| 20161 | aacaaaaaaa cacaaaaaac caaaaataaa aaatctgggt gaggtatttg caatactaga |
| 20221 | gccaaagaat ttgacctaag aggccgggcg tggtggttca catctgtaat cccagtactt |
| 20281 | tgggaggccg aggtgggtgg atcacttgag gccagaagtt tgaaactaac ctggccaact |
| 20341 | tgttgaaacc ccatgtctac taaatacaca caaaaaaggg cccggcgcgg tggctcgtgc |
| 20401 | ctgtaattcc agtactttgg gaggctgagg tgggaggatc acctgaggtc aggagatgga |
| 20461 | gaccatcctg gctaacatgg tgaaaccccg tctctgctaa aaatacacaa attagccggg |
| 20521 | tgtggtggca tgtgcctgaa gtcccagcta cttggagggt gaggcaggag aatcacttca |
| 20581 | acctaggagg tggaggttgc agtgagctga ggtagcgcca ctgcactcca gcctgggcga |
| 20641 | cagagcaaga ctctgtctca aaataaacaa ttaactgggt gtggtggcac acacctgtaa |
| 20701 | tctcagctgc tcctgagcct gaggtgggag aatcacttga acccgggagg cagaggttgc |
| 20761 | agtgagctga cattgtgcca ctgcgctcca gcctaggggg gagaaaaaga aaaaaaagaa |
| 20821 | ttctgatcca ggcactgact cattgagcaa gtcatcttta ctgttcaggc ctcagctttc |
| 20881 | ttcctttgtt tttttttgag acagagtctc attctattgc ccaggctgga gtgcaatggc |
| 20941 | atgatttcag ctcactgcaa cctctgccgc ccgggttcaa gcaattcttc tgcctcagcc |
| 21001 | tcccgagtag gtgggattac aggtgcctgc caccgcgccc agctaatttt tgtactttta |
| 21061 | gtagagatgg gggtttcact atcttggcca ggcbggtctc gaactcctga cctcatgatc |
| 21121 | cacccacctt ggcctcccaa agtgctggga ttacagatat gagccaccgc acccagctag |
| 21181 | gcctcagttt tctaatgtgt aaaatgaaaa tatgcactag accttcactt gtttttttct |
| 21241 | atgtgataat tttttttaaa aaaagaaggt gtcatgttac attttatctt tatgtttaca |
| 21301 | tttcatcttg taaggttgcc tttgagcctt gacacagtga tttttctcat tcaactagtt |
| 21361 | tctctcccat ctcaactagt gacctcacca tccactaatt gtttaagcca gaaattggga |
| 21421 | acttttcctt ggttcctctt ccttgtttac ttcttccatc taccccatga gcaaatccta |
| 21481 | tctgttcttc cttaaattta gttcccagat ctatctcctg catctcaggt ctatgtactt |
| 21541 | cttcccatct cccctgctac tagtctgtcc agagctacaa ccacttccct tatagattat |
| 21601 | tggaataacc ttcttattgg tcttcatatt ttcactctga ctcccctctg atcaattttc |
| 21661 | tgcaatgcag ccagaattat cttttaaaaa tgtaagtttg agccaggcac agtggctcac |
| 21721 | gcctgtaatc ccagcacttt gggaggccaa ggtgggagga tcacctgagg tcaggagttt |
| 21781 | gagatcagcc tggccaacat ggtgaaactc tgtctctact aaaaatacaa aaattagctg |
| 21841 | ggcatggtgg tgggtgcctg taatcccagc tacttgggag gctgaggcag aactgcttga |
| 21901 | acctgggagg ccatatatat ataaagtttg atcacaccac tgtcctgctt aatctttttt |
| 21961 | tattttttaa cttttaaatt ttttaagaga agggtctctc tgtgttgccc agactggtct |
| 22021 | ctaattcgtg ggctcaagag atcctctcat cttagcctcc caagtagctg ggaatacagg |
| 22081 | catgtgccac cacacccagt gactttcctt cctaaaatgc ttcaatagtt tctttttctt |
| 22141 | tttctttctt tctttttttt tttttttttg agatggagtc tcgttctttc acccaggctg |
| 22201 | gagtgcagtg gcgtgatctt ggctcactgc aacctctgcc tcccgggtta aagtgattct |
| 22261 | tctgcctcag tcttccaagt agcbgggact acagcatgtg ccaccacacc tcaataattt |
| 22321 | tttgtatttt tagtagaaat gggtttcacc atgttggtca ggcbggtctc gaacagctga |
| 22381 | gcttgtgatc cacccgcctc ggcctcccaa agtgctggga ttacaggtgt gagccactgc |
| 22441 | acccagccac ttcaatagtt tctttttgtg cttaacataa agtttaaatt catgtccaga |
| 22501 | gcctataagg tgtcatatga ccaggcccct caaggtatat ccacttcact caagccatgt |
| 22561 | ttgtctcatc ttaattcttc aaacacccca cattctttcc taactcaagg tctttgagct |
| 22621 | tgctgcttct tccaggtgga ctgatcttag cccttccctt tgttgttgat tttttaactt |
| 22681 | tttttttttt ttttttgtga gaaagtcttg ctcttgtttt ccaggctgga gtgcagtggt |
| 22741 | gcaatctcgg ctcactgcaa cctccacctc ctgggttcaa gtgagtctcc tgactcagcg |
| 22801 | tcctgagtaa ctgggattac aggcacctgc caccacaccc ggctaatttt tgtattttta |
| 22861 | gtagaaatgg ggttttgccg tgttggccag gctgatcttg aactcctgac ctcaggtgat |
| 22921 | cctcccacct cggcctccca aagtgctggg attacaggca tgagccagcg ctcccagcct |
| 22981 | ttttaccttt ttttttttta atttcaatag gtttttgggg aacaggtagt ttttggttac |
| 23041 | gtgaaaagtt ctttagttgt gatttctgag attttagcac acccatcacc tgagcagcgt |
| 23101 | acactctacc caatgtgtag ctttttatcc ttcacccccc caagtctttc cctgagttcc |
| 23161 | caaagttcat tgtatcattc ttatgccttt gcattctcgt agcttagctc ccacttacga |
| 23221 | gtgagaacat acaatatttg gttttccatt cctgagtctc caattccacc caggttgctg |
| 23281 | tgaatgccat taatatgttc ctttttatgg ctgagtagta tttcatgata tatatataca |
| 23341 | cacacacaca cacacacaca catatataca cacacacatt tatatgtgta tatatacaca |
| 23401 | tatatatata tgtcacattt ctttatccac tcattgattg atgggcattt gtagccctgc |
| 23461 | cctttgaatg gctgacttct tcttatgctt cagaattaat actttgtaat tacattaata |
| 23521 | tgtttttttt aaaaaaaaac cttttacata tatattggtc tgtcacaata atgtataaca |
| 23581 | atattatagt ttctgtttta atgacttaac tttggcaaac tggtcaatga tttcattaaa |
| 23641 | actgatcttt ttaaaaataa tcagcttcaa tagacagttt aaccagattt gtcaactttt |
| 23701 | tgtttttggc acagtctcac tctgttgccc aggatggagt gcagtggtgc catcttggct |
| 23761 | cactgcaacc tctgcctcct gggttcaagt gattctcatg cctcagcctc ccgagtagct |
| 23821 | gggattagag gcacctgcca ccatgcccag ctaagttttg aatttttagt agagataggg |
| 23881 | tttcaccatg ttcgccaggc tggtctcgaa tgcctgacct caagtgatct ggctccctcg |
| 23941 | aactcccaga gtgctgggat tacaggcgtg agccaccaca cccggtctcc ataaaaaaat |
| 24001 | cttaaacaaa aggatatggt tggtgaaata caaatcctat tgcgcagtaa ttttcacaaa |
| 24061 | ttttaaaatt gacaatacct tcgcttcttt cagatcaatt atagtggctt tcaaatgtca |
| 24121 | tcgctcaaaa aagttcaaat gtcatcgctc aaaaaaaaaa caaaccttca ctagaaaatt |
| 24181 | aatcataaat tgatggtaca gactgggtgc agtggctcac aactgtaatc ccaccacttt |
| 24241 | gggaggccga ggtggatgga tcacctgagg tcaggagttc aagaccagca tggccaacat |
| 24301 | ggtgacaccc cagctctact aaaaaaaaaa aaaaatgaaa aaattagccc ggcgtggtgg |
| 24361 | tgcatgtctg taatcccagc tacttgagag gctgagacag gagaatcgct tgaatgcaga |
| 24421 | aggcggaggt tgcagtgagt cgagatcata acactgtaat ccagcctggg caacagaggg |
| 24481 | agactcctca aaaaatataa aaataaataa attgatgtta catttggaaa aactgctgaa |
| 24541 | attttcttct tctgcaaaaa gttagaaaaa accactgaat gattgaaaac attgacatta |
| 24601 | tttaattctt tgctttagag caagtactca gttcttgatt gaagtgattg agggattcaa |
| 24661 | acattgtcct tttgttttct tccggtaacg tcagaccagc atattttgtt attgctgctg |
| 24721 | gctgttttct tcaaaatttg ctttgaaaaa gatgtaaatg gccatttcct tatacttgtg |
| 24781 | attaaatatt tgttggcctt tccaccagtt ctctgcactg atcttcaaga aaaaatttta |
| 24841 | aaacctgcac acaaaatttt aaagactgaa cacagaattt taaagcatgc acatttttca |
| 24901 | aagcbgtttc tatctctttg gaagtttgtg tctgattaag ttcatctaag acttcactcc |
| 24961 | aggaaaaaag ttaacccaga aaagataaat agcctcagca gagagaaaaa tctactgatt |
| 25021 | ttcttgtgtc tacattttgt tttagatagc ataatacttc aaaagttggt actcatttct |
| 25081 | taaactttac tgtgtgcata gcttcaaaat gaatactttg ttgtgtgtgg gaaagccttt |
| 25141 | tcattgttat acttacaatc ttcagcatgg tccatccgtg aatctttttt aaaattgata |
| 25201 | aaaaggccag gcgcgatggc tcatgcccgt aatcccagca ccttgggagg ccaaggcaag |
| 25261 | tggatcacct gaggtcagga gtttgagacc agcctggcca acatggtgaa acctcgtctc |
| 25321 | tactgaaaat acaaaaatca gcbgggtgcg ttggcgtgcg cctgtaatcc cagctactca |
| 25381 | ggaggatgag gcaggagagt ggtttaaacc tgggaggcgg aggttgcagt gagccgagat |
| 25441 | tgtgtcactg cactccagcc tgggcaacag agcgagactc cttctcaaaa aaaaaaaaaa |
| 25501 | aaatggttaa aaagatgaaa taaaaatttt ccaagttccc ctcaaccccc ctaaaaaaaa |
| 25561 | atcatacatg aagaaacatt tccaaaacag tggagtttgg agattgattt gcacaagcac |
| 25621 | aaatgaggat ttaggataaa tttctttgac ttttacctga acactacagt cagtcttggc |
| 25681 | catagtttca gcattgccat gtccttggtc atgatagagg ttcatgtcca gcatatcaca |
| 25741 | attcagtcat atggtgacta ttatgtgagt tcaggagtgt ttatcctaga aatagaaaga |
| 25801 | cacccaggaa tgactccttt ctttcaaatt tgctgttctc aaaatgaaca tacttaagga |
| 25861 | ctttacattt gatcagggta taaaccactg ggaagactaa gtgctcagaa aaagaaccaa |
| 25921 | agacttggag ataggaacag agcctgaaat gaacagaaac tattctatac cccccatgaa |
| 25981 | cctagaaaca aatgtgtata gctaagcgta ttagtccact cgggatgctt taacagaata |
| 26041 | tcaataaact ggaatgcttc acagtaaaca tttatttctc acagttctgg aggctgggaa |
| 26101 | gtccaagatc tggtatctgc tgaggttcct cttactggtt tgcagatggc cgtcttcttg |
| 26161 | ttctatcctc acatggcaga gagcagagaa cagaccatct cttgtgtctg ttcttagaag |
| 26221 | aacattaatc ctatttatga gcactctacc ttcgcaacca aaaggcttca tctccaaata |
| 26281 | ccatcacttg gagatttagc cttcaacaca ggaattttgg gggtacacaa acattcaatc |
| 26341 | catagcactg agtctgtatg tgttgggaac tgattgataa attggaattc ttcaaattaa |
| 26401 | cttacatgta gaatacagta tttattaaag gataagtaaa aagaaatgtg ttaatttata |
| 26461 | gtgtatttat tattattaaa agtctatagg gcbgggcaca gtggctcatg cctgtaatcc |
| 26521 | cagcactttg ggaggccaag gtggggtgga tcacgaggcc aggagtttca agatcagcct |
| 26581 | ggccaagatg gtaaaattcc atctctacta aaaatacaaa aattaattgg gcatggtcag |
| 26641 | gcgcacgcct gtaatcccag ctacttggga ggctgaggca ggagaactgc ttcaatgaac |
| 26701 | ccgggaggtt cattcaagat cacgccactg cacttcagcc tgggtgacag agtgagactc |
| 26761 | tgtcttaaaa aaaaaaaaaa aaaagtctac agtaactgca attgtttaga atttaagcca |
| 26821 | acaagatatg ttttcttttc cgggagttgt attttttatc actgattgtc tacaattgac |
| 26881 | agtgcatgtc aaaaagcaga cttgtcgtgc tgcagtggct cacgcctgta atcccagaac |
| 26941 | tttgagaggg cgaggcagga ggattgcttg agcccaggag ttggagacca acctgggcaa |
| 27001 | catagtgaga tcccatctct aaaaaaataa aaataaaacc actcattttt gtcaatttta |
| 27061 | ttactgcttt taatttttct aaagacgatg tgctgctcca gctctgggta cacacatgcc |
| 27121 | cacgagagca agctcatgag agaagaaact acatcccctc tatccaccta tatttctaac |
| 27181 | gccttgcaca gtagctggca catagtggac atgaaataaa tatttgtgga ataaataagt |
| 27241 | gaagaagaat tttagaatac tttataagaa acaataaatc ttcagagatc aacatttcaa |
| 27301 | attagtaact ttatttacaa acctctcttc tgggttttat tatttactac aagtgcttgc |
| 27361 | aagacctgtt attgtacact catgtattcc tatcaaaggt aatcatcaga cattatattt |
| 27421 | cattaaataa tttaatggtg ctttctgttc tttattcatt aatttttttg tgtgtgtgtg |
| 27481 | agaatggagt ttcactatgt tgcccaggct ggccttgaac tccagggttc aagtggatcc |
| 27541 | tcctgccttg gcctcccaaa gtgctgggat tacaggcatg aaccaccatg tccaacaatg |
| 27601 | ttttctgctc tttagtaagt ctagccatat taaattttat gtgttttttt tttctttttt |
| 27661 | gagactgagt ctcactctgt cacccaggct ggagtgcatt ggcgcgatct ccactcagtg |
| 27721 | caacctccgc ctcccagttt caagcaattc tcttacttca gcctcctgag tagctaggat |
| 27781 | tacaggcacg taccaccaca tctggctatt tttttttttt ttttttttga gacgaagtct |
| 27841 | cactctgttg cccgagctgg attgcagtgg catgatcttg gctcactgca acctccacct |
| 27901 | cctgggttca agtgattctc ctgcctcagc ctcctgagta gcbgggacta caggcatgtg |
| 27961 | ccaccatgcc cggctgattt ttgtatattt tttaacagag atggggtttc attatgttgg |
| 28021 | ccaggttggt ctcgaactcc tgacctcgtg atccacccac cttggcctcc cgaagtgctg |
| 28081 | ggattacaga tgtgagccac cgagcccggc ctaatttttg tgtttttggt ggagatgggg |
| 28141 | ttttaccatg ttggccaggc tggtctcgag ctccggacct caggccatcc acctacctcg |
| 28201 | gcttcccaaa gtgctaggat tacaggcgtg agccactgtg cccagctcat attacatttt |
| 28261 | aaatggaggt agaatatcaa cctgaattca ctaagagaat gcaggacaaa ttgccctgat |
| 28321 | atcctgacac aaaggtgaag caggactttt ttgtaatttg aggatttaaa agcatggttg |
| 28381 | ctccaggcca ggcgtggtgg ctcctgcctg taatcccagc actttgggaa gccgaggcag |
| 28441 | gcggatcatt tgaggtcagg agttggagac cagcctggcc aaaatggtga aaccctgtct |
| 28501 | ctactaaaaa tacaaaaatt agcbgggcgt ggtcaggcgc ctgtaatccc agctacttgg |
| 28561 | gaggctgagg caggagaatt gcttcaagga acccaggaag cagaggttgc agagcccata |
| 28621 | tcgtgccact gcactctagc ctaggtgaca gagcgagacc agcctggcca acatggtaaa |
| 28681 | accccgtttc tactaaaaat acaaaaaatt agcagggcaa gctggcgggc gcctgtaatc |
| 28741 | ccagctacac gggaggctga ggcaggagaa tggcgtgaac ccaggaggcg gagcttgcag |
| 28801 | tgagctgaga tggcgccact gcactccagc ctgggcgaca gagggagact ccatctcaaa |
| 28861 | aaaaaaaaaa aaaaaaaaag tatggttgtt ccagtcctgg gaaattgaaa tagggggtaa |
| 28921 | aaaggctggt gggtgtggta ggcagaataa ttacttcttc cctctcccaa gatgttcagg |
| 28981 | tcctattccc catcctattc cctggaacct atagctagat tgccttatgg ggcaaaagga |
| 29041 | atttttcaag tgtgactaaa tttagaatca tgagttagag agattatcct ggattatctg |
| 29101 | tgtgggttta atgtaatcac aggggtccct agaaagaaaa gagggaagca gaaaagccaa |
| 29161 | aggagatgga atgatgggag ttgagatcag tcagatttac agatgcaata ttgtttccta |
| 29221 | taaattctgt ggtgaaagaa atgctatatt gttgctggct ttgaagatgg aggaaggggc |
| 29281 | tgagagtgaa ggaatgcaag tgaccttaaa ttggaaaaga caagaaaata attggccctg |
| 29341 | tagaggttat aaaagaaggg tggtccttct gacacactga ttttagtccg atgagattga |
| 29401 | ttttgaactt ctgacttcca aaaactgtaa gacaatacat tagtgttatt gtaaaccact |
| 29461 | acatttttgg taatttgtta cagcaatagg aaacttatat agtgggccct gggatccatc |
| 29521 | tgccctagcc gtttatctaa aacaaaagat cttataggga aagcaaacaa acaaacaaaa |
| 29581 | aatacagaaa caaagcaaaa caaagcaccc ctatgctctg agcttgggcc tggtaagggc |
| 29641 | actatgagcc catgatttag catgttgctg ggaaatctcc ttccccctgt tgagttgcca |
| 29701 | gaatcgtgga agcagatggt gtgggggatt caggctcctc cactgctttt ctcagccaga |
| 29761 | cctttgagga gagcttggct gaagtcttcc aagctttatc cccttgtagc cattgttttg |
| 29821 | agccatctca aacattaagt agctcttgag ggttgaattg agtccttcct aaagacagac |
| 29881 | taaagtccta gtctccagga cctgtaaatg ttcccttatt tgtaattagg atgtttacag |
| 29941 | atgtaatcaa attaaaatga ggtcataccg gactagggtg ggctctgatc caatatggcc |
| 30001 | ggcatcctca taagaagagg agaagagaca tagacacata gggaaaagac cgtgtgatga |
| 30061 | tggcagagat tagagtgatg tgtttacaag ccaaggaatg tcaaatattg ccggcaatat |
| 30121 | cagaaactaa gaaaaaggca tggcacagat tgtctctgag agcctctcag agagtatggc |
| 30181 | cctgctgaca ccttagtttc agaggtctag catccagaac tctgagagaa cagatttgtg |
| 30241 | ttgttttaag acacccacca tgtggtaatt tattacagta gcccttaaac taacacagtg |
| 30301 | accttgtgtc ttctgccaga gggatttgat cacactggtg aggaagggtc ctggtccata |
| 30361 | ataacccaaa tttctgccag ggaagaggag attgaaaggt cccagtgatc ttggcctatt |
| 30421 | tacttgggag ctcttccaca gaacaaatga actcctgaag atgagctgct acaaggattc |
| 30481 | tgtgctttgg acagaattgc acttgtctct tccccctgct gtcactccct gactaggaaa |
| 30541 | ggtccaagct gtgaaggaat cccccccaga agagacttgt aagcattaaa gacctgtgga |
| 30601 | aaactaagag ggagggtgga tagaggagtc tggagaccga ctaaccttct gagctgctta |
| 30661 | ttgcctgaag tgggggtgga gtgggctgca aacactgttg tatttggggg acacatcaca |
| 30721 | gatgcttatt tgcaccttgg actttgtcag gacactctgg ttacagcaca actttgtgaa |
| 30781 | catggatcac aagatacttg gaaaagcagt gtttgcatta atcagattag gattagctaa |
| 30841 | aattttaatt aaagaaccag agagtacttc agaggcaagg agatgatagt tacctgggat |
| 30901 | tacttgctat ttgtgaaacc aaccagctga ccacttgtgt tgtttgtccc aaggtataat |
| 30961 | aataactaga tgtttgcata ttgtgattgt ttctgttcgg atttaatggc tgatctattt |
| 31021 | atcatattaa aagagggatc aagttcaaag atttcaatgc tctttatctt ctttctatat |
| 31081 | atgatattta tttttaaatg tccatattaa atatacatat atacacacga ttctcagaat |
| 31141 | tacgaagaat gtgcbgtgta gcattcaatt atatatataa tttttttttt gagatggagt |
| 31201 | ttccctcttg ttgcccaggc tggagtgcaa cggcatgatc tcgggtcaca gcaacctctg |
| 31261 | cctcccaggt tcaagtgatt ctcctacctc agcctcctga gtagcbggga ttacaggcat |
| 31321 | gtgccaccat gcbgggctaa ttttgtattt ttagtagaga cggggtttct ccatgttggt |
| 31381 | caggctggtc tcgaactccc aacctcaggt gatccaccgg cctctgcttc acaaagtgct |
| 31441 | gggattatag gcgtgagtca ccaagcctgg ctccaattct gtatattttt aaatgtaagt |
| 31501 | ataaagatca aagatacaaa gtatattcac atatcagttt ccatgttgtt attattatta |
| 31561 | ttatttgcca ttttaaccat ctccagatga agaaaactga atgtaaagtt aaatatctcc |
| 31621 | ttaactttta ctctatttaa aaagagactg aattatttgt gaggtaaccc agttgtagct |
| 31681 | gttctactct tctctactgc tagattttgg aagaaccaaa aaggaataaa gcaaagcatg |
| 31741 | acacagaaga gaaagcagaa ttatactgta gtagtatgct catattttat cagagtttgc |
| 31801 | ctgtactttt gctgattttt ttttctccta gcttggacat tttaaaggct tattttaaat |
| 31861 | ttatctcttc tgtattaaca ggtgagaagt ggacaaatca atgtataaaa actactatgg |
| 31921 | ctatcatcat ttagaatttt gtcttaaaag taattggatt tctggggtcc tagacacata |
| 31981 | atgattagag atcatgccac cagttctata tggagccatc tcaattcatc ctttctcaat |
| 32041 | ttatcctttc caggcatttt tcttgaaact gcctaagtac tggcgtctcc ctacgactat |
| 32101 | tatatctgaa aacacattca gttttcaaac cagctgaatt ttatgcctga ctattccttt |
| 32161 | taacttatac caatgaaaat cctttaataa attcactcac aattgctcca ctttctcaaa |
| 32221 | cagagcacaa agcatctaac aatatttctt catgattgat agcatacaca ctcttgactt |
| 32281 | catattcttc aaaatcttgg tatttttata agcattagtg cctttttttt ccccagtcag |
| 32341 | aagcactgac ggatttattc tattgggtgt tgtcttctgt ttggtcttta gcttttctta |
| 32401 | gctgtgatcg tttattgctg aataattaag ggcaattctt tcccactgaa atacttttca |
| 32461 | agttaaacat tttttcctta aatttttgcc ccaggctact tctagatcca gcgttgttcc |
| 32521 | agcttactga gcattccaga gggctaaggt ccaagttagc caaggtttta aaatgttgac |
| 32581 | catgtggacc tgaattcttt ccagttacat atttgcatac ttttaacagg agtggttatt |
| 32641 | tctcgtcaaa agacagaacg ctggaaatac cagggcaaaa ctatgtgttt ttttcttggt |
| 32701 | gatatcttga tcttagcatt ttactctctc ttcctggttt ctagcttgac ctttgtattt |
| 32761 | tagaggagag taatagagta acagtgcact gagaagcaat gggacatgac agaatagttt |
| 32821 | cagcagtgaa agtcagggaa gctggtttca accattcatt cacaagctta aaggactttg |
| 32881 | gtatctgact ttgtggatgc cactttctca tctgtaaaat aagggggttg gatcgtgtga |
| 32941 | tttataaggg gctttcttac tttctaacat tccatattct ataaagacag ttaaagtctc |
| 33001 | cagagagtaa agaatgtaga aaattcctga aatttactca gctcctttat tcctaaatat |
| 33061 | ttttgaattc tgaaattatt attattatta ttttttgaga tggagtctca ctctgctgcc |
| 33121 | caggccaaag tgcgatggca caatctcagc tcactgcaat ctctgctgcc caggttcaag |
| 33181 | tgattctcct gcctcagtct tctgagtagc tgggattaca ggcatgtgcc accatgcctg |
| 33241 | gctaactttt gtattttcag tagagaaggg gtttcaccat gttggacagg gtagtatcaa |
| 33301 | atttctgacc tcaagtgatc cacccgcctc ggcctcccaa agtgctggag ttacagccaa |
| 33361 | agcacctgac ctgaattctg agttttgatt ctgttttttt tttttttttt ttgagacaga |
| 33421 | gttttgctct tgtcacccag gctggagtgc aattgcacgt tctccgctca cagcaacctc |
| 33481 | catctcccgg gttcaagtga ttctcctgcc tcagcctccc aagtagatgg gactaccacc |
| 33541 | gtgcctagct aataattttg tgtattttag tagagatggg atttcactgt gttagccagg |
| 33601 | atggtcttga tctcttgacc tcgtgaccct ccctccttgg cctcccaaag tgctgggatt |
| 33661 | acaggcgtga gatactgcgc ccggcccata ctcacagatt tctttgtgat attatctcat |
| 33721 | ttgttttcac cacacccaga tgatgtggat ggtaggagaa atcccatgcg aataagtgga |
| 33781 | atatgttagt aactgcttcc ttgggtcctt ttgcctctgg acttaactct ttttttttga |
| 33841 | gacggagtct tgctctgtca cccatgctgg agtgcagtgg cgcaatcttg gctcactgca |
| 33901 | acctctgcct cccagattca agcaattctc ctgcctcagc ctcccgagta gctgggatta |
| 33961 | caggcatgtg ctgccacgcc tggctaattt ttttgtattt ttagtagaga tggggtttca |
| 34021 | ccatattccc caggctggtc tcaaactcct gaccttgtga tccacccacc ttgaccaccc |
| 34081 | aaagtgctgg gattagaggc atgagccact gcacctggcc tggacttaac tcttaaatga |
| 34141 | tgttacccag gaaaaccaat ctatgactaa ggcaaaaaaa aaaaaaaaaa aaaaaaaaaa |
| 34201 | aaaagttgca cagagaagag tggaaatatc agtaaatcag aagctaggca ggcctaatcc |
| 34261 | ctaatccact tggtgacctt ctgcaaaaca cctttctgtt agccttttca cccgtgaatt |
| 34321 | tttttttttt tttgagacgg agtttcgctt tggttagcca gactggagta caatggtgcg |
| 34381 | atctcagctc actgcaacct tcgccttctg ggttcaaggg attctcctgc ctcagcctcc |
| 34441 | tgagtagctg ggattacagg aacctgccat cacacccaga taatttttgt atttttagta |
| 34501 | gagatggggt ttcaccgtgt tggccaggct ggtcttgaac tcctgacctc aggtgatcca |
| 34561 | ccttcctcgg ctgcccaaag tgctgggatt acaggcgtga gtcactgtgc ctggcttact |
| 34621 | tgtgaaatta aagcttcaac ttttactcct taggaacgtc tcacaaattt ccacacataa |
| 34681 | atctaaagta gacactcctg cttgccaaaa gcatactctc taaaacattg gttctcagac |
| 34741 | ggacatagtg gctcatgcct gtaatcccag cactttggga ggccgagacg ggtggatcac |
| 34801 | ttgagatcag gagtttgaaa ccagcatggc caacatcgtg aaaccccatt tctactgaaa |
| 34861 | atacaaaaaa aattagccag gtatggtggt gcgtgcctgt aatcccagct actcgggagg |
| 34921 | ccgaagcagg agaatcactt gaacctggga ggcggaggtt gcagtgagcc agatcccgcc |
| 34981 | actgcactct agcctgggag atagagcgag actctgtctt ggaaacaaac aaacaaacaa |
| 35041 | acaaacaaaa aaattggttc ttgagcaggg caatttcacc tccaagggga catttgacaa |
| 35101 | tggcbgggga catttttggt tgtcacatct ggggttgggg agtgaagtgc tattggcatc |
| 35161 | taatacgtag aggccaggga tgctgctgaa catcctgcag tgcacaggac agcccctgac |
| 35221 | agcattaaac agtccaaaat gtcaattgtt ttttgaggtt gagaaacgct ggtctaaaag |
| 35281 | aaatgacagg acaggtggct tggctcatgc ctatcatccc agcactttgg gaggctgagg |
| 35341 | cgagtggatc acctgaggcc agtagcttga gaccagcctg gccaacatgg tgaaacctct |
| 35401 | tctctactaa aaatacaaac aatagccggg cgtggtggcc cacacctgta atcccagcta |
| 35461 | ttcagaaggc caaggcagga gaatcgcttg aacccggggg gcaaagattg cagtgagcta |
| 35521 | agatcatgcc actgcactcc agcctgggtg acagagtgag actctcaaaa aaacaaacaa |
| 35581 | acaaaaaaac agaaatgact caaatgttta cagtgattac ttctggatgg gattatgata |
| 35641 | attgtttctc tactgttcta aaaaactttc tgttctttcc aaatgtttaa tattgagcac |
| 35701 | ttattttaca atcattaaac aaagaaatga caacaactgt taaaaaaaaa ttcttaagaa |
| 35761 | ctgaaaggca aacaccacca ccactaaagc catgtccccc agatctaggt tgtgagtcac |
| 35821 | atgataccga cttcagaggc agcatgtctc aaggcctctc caatgtgtgt cagggacagc |
| 35881 | agtgggagta gggccctctg agtcagcctc tttgacctgg gatgctgcag gagatcttag |
| 35941 | caaacattca agattttcac tgagtcattt gcctgtggca ttgatctgct cactcaagtc |
| 36001 | ttccggtgtt ttggtgtgag ggaggctaaa gaggaaaaac agcbgtttag tttctcattg |
| 36061 | atttgtattg tcattgagca atacctggag cccaagtgga tgtcttttat tctgtggcct |
| 36121 | ccacagctgg gtcagtgtgg aaggcttcag accatgagaa attgttcctg ggagtcaatt |
| 36181 | attttaataa gtgagatatc agagtatatt cacagaaaga ggcttccctg gcactgtaag |
| 36241 | ggcagccagg aataaaataa aaatgcttct tcagtgaaga taattgtcca aattgaagac |
| 36301 | ataggaaata tttctaattt gcctgtgtat ttggtgaaca tttctctctc tctctctctt |
| 36361 | tctctctttc tctctctctc tctctttctg tctccataaa tgaagacatg ggaaaatcta |
| 36421 | ttaaaaatta aattaaaatg caacactttt acataaacaa aattcccaat cactctcctt |
| 36481 | actttacctc agcccaacca aatgtgagtt aaatctcttc cctttcccat gggacactag |
| 36541 | catcttggtg gcccagctca gtagctaagc atagagtaat gagtttgtta aataatttca |
| 36601 | attacattgt ttttgagatt ccagggtcac agggaaattc tgacctgaat tatctggatg |
| 36661 | aactctcaca attgtgcact gccttgttca aatttgggaa gtgctgagaa gcactcatcc |
| 36721 | aaggggtaga tctggacttc agctcatgca gcataataga atttcaggct cagtgatttc |
| 36781 | taatctgtta ccacttgcca ttttctactt gaagagggca gacaccttca ctgatctctg |
| 36841 | tttctatttc atcttttgaa aaatcgggat gataatggtt atctggttta catgctttta |
| 36901 | catgcttgtc aggagttatt ttaaagcact ttacaaatat aaaaatgtac aataaaattg |
| 36961 | cagagtcgga tgcagcaacc cctgtatcct ttgaggaatc tatcacaatt catacccctc |
| 37021 | ccaagccaac ttttctggta ctactctgga aaattgtgct tatacagtta aaatttttat |
| 37081 | tttcaaaaag ttgtgtaatt ccctttttca accatttttt tttgttataa gaaatgcaat |
| 37141 | taaaaacata cttttctttc caaatagaag agcctattct gccttataag aattgtttga |
| 37201 | aagattgaag gacaaatgaa gtcatacaac ttctctgata ccatattgaa ctcaaatgaa |
| 37261 | cacattttga ggtaagaaat gagcctggaa gatttttctt tttttttttt ttgagatgga |
| 37321 | gtcttgcttc gtcgcccagg ctggagtgca atggcgcaat cttggctcac tgcaacctcc |
| 37381 | gcctcctggg ttcaagtgat tctcctgcct cagcctcctg agtagcbggg attacaggag |
| 37441 | tgctccacca ggcctcacta attttttgta tttttagtac agatgggttt tcaccatgtt |
| 37501 | ggccaggttg gtctcagact cctgacctca tgatccaccc gccttggctt cccaaagtgc |
| 37561 | tgggattaca ggcgtgagcc actgtgcctg gcaagagata gaatttttca caaaaataac |
| 37621 | tcataccaga aactttgtag gcccccaccg atgcccataa tctttaggaa acaaactcta |
| 37681 | gtgtcaccta tttcctgata ccttctgtaa cgaccaggtc atattctgag ttcctacagc |
| 37741 | actcagtgtt tctcatcgga gctgtagcaa tggagtaata aatggcactg ctcaggaaga |
| 37801 | gagaattcag aaagaaaaga gcagggccta gaaaaggaac aaacttatcg gatgtgtaga |
| 37861 | gaaagaatct gcaaagcaag taataaaatc cagaaaaacg tggtatcatg gtggtcacaa |
| 37921 | aaaaaagagt ggttaaattt tcgaaaatgg agacaaacca ctcaccatga tcgtgccatc |
| 37981 | tgaatagaaa cacagaaatg atgacaaaaa aatggctaaa gaaagaaggt caacaaccat |
| 38041 | gtccaatgct ttggatatta agaagtgttc agatggattt agcaagtaga aagtcactga |
| 38101 | cacttggtgc acaaccagac ctcaatgtat ttaggaggaa actgttattt tggaacaatt |
| 38161 | gaaagctttc atttggctga gaaatatcaa ctcattctta aataggtatg ttagaaggag |
| 38221 | cctctgggcc tgatatctcc ttgattacaa ggaaaatgaa taatagttgc gtctgataac |
| 38281 | catgacatat cacccctatg tttgcttgta gctttcagca gctcagaaca ttgttgaaaa |
| 38341 | cactcattat cccaatatcc ccaacaggga agtgttaaaa cctgccttgt gcaactgtca |
| 38401 | atctctgatg attagataaa aacaattatg tcatgagttg tagctattgt tatttacttc |
| 38461 | ccaagagcca gattacacag gtatctgatt gctgaagcaa tgtctggatg taccacgaaa |
| 38521 | ttccacatgc atgtacagta taaacacctc tggctatcac cttatctaat tttgatcaga |
| 38581 | tacaggttaa tcccctcctg tatctttata tgtcctgaat ttgcctcatc cacctgaact |
| 38641 | ttgccataat ttactctcaa tttgtatgtg tctgtgatgg agttgttgtc attttccttt |
| 38701 | tctttttctt ttttttgagg ccaagtctct ttctgtcacc cagactggag tgcaatgacg |
| 38761 | cgatctcagc tcactgcaac ctctgcctcc cgggttcaag taattcccct gcctcagcct |
| 38821 | cctaaatagc tgggactaca ggtgcatgcc accatgcctg gctaactttt tgtattttta |
| 38881 | gtagagacgg ggttttaccg tgttagccag gatggtctct tgatccgccc gcctccgcct |
| 38941 | cccaaagtgc tgggattaca ggcatgagcc accgcgcccg gcctcatttt ctctatctgt |
| 39001 | ctgcttaatc aggttttgca agtttcacat ttcctcaact aaacttcatt ttaccaccca |
| 39061 | agcaaacttc attataatta ttttattttt ctgatgacat cagatttctg taggggtacc |
| 39121 | tattagctag agatacttag attttatttt cccagcatgt gactatttcc cattctatac |
| 39181 | aacacaatgg caagagcagg agtatgtttt tggacaatta aagagagaaa aggccgggcg |
| 39241 | cggtggccca tgcctgtaat cccagcactt tgggaggctg aggtgggcag atcatgaggt |
| 39301 | caggagatca ggagattgag accatcctgg ctaacacggt gaaaccccat ctctactaaa |
| 39361 | aatgcaaaaa attagccggg cgtggtggtg ggcacctgta gtcccagcta ctctggaggg |
| 39421 | tgaggcagaa tggcgtgaac ccgggaggca cagcttgcag tgagccgaga tcgtgccact |
| 39481 | gcactccagc ctgggcggca gagcgagact ccgtctcaaa aaaaaaaaaa aaaaaaaaaa |
| 39541 | gagagagaga aaataactca gcttcattta gatttctatt attcttctct gatccatata |
| 39601 | gtggatgttc acagaagtgt aggaaccaaa actatgcaag ttaaactgtt aattttagaa |
| 39661 | gtttatagca gtgatgtgga aggctgacca aagttggcgc tttggtctca ctataagcca |
| 39721 | tagtaataaa tacattaaca atgtccacaa acaacagtag acaccattta gtaagggctt |
| 39781 | gctccaagcc aggcactgtt ttcagcttaa gtctcacagc cttgtgaagt agattctgtc |
| 39841 | ccatcttaga gcaagttggt ctgaggatcc tcaagggtaa ggtctcttgc caaaggtcag |
| 39901 | cagaggtagt gagttcagaa ctgggattta aatccaggct gttctatttc tattccacaa |
| 39961 | ttctgcctca actgcttctc tacattttag gcacacgttt gaatactgaa aatttgggtc |
| 40021 | acagagttgg agatgtttct tcttctaggc cccaaaccgg agcggggtgc agtggctcaa |
| 40081 | gcctgtaatc cgcactactt gggaagctga ggcgggagga tcgcttgagc tcaggagttt |
| 40141 | gaagatacag tcagctatga tcctgccact gcactctagc ctgggcaaca gaacaagatc |
| 40201 | ctatctctaa aaaaaaaaaa aaattaaaat tttaacacaa gggcccaaac aagtcaacag |
| 40261 | taattacaac atgatcttta ctactgtcta ataacatctc acagtgcttt aaacatacgt |
| 40321 | agttaccatt ctgtccactt ggttgcctgt aatattaact cagtaagtag gaatacttcc |
| 40381 | tgccctgact aatatactca taaatatatg ctctaagtca agggtcttgc aggttctgat |
| 40441 | ttaaaaaaca caattttttt gagggtcact ggctttacat tatctcattc ttttcaagtt |
| 40501 | cctttgtacg taaaacatcc tatgctcagt tatatgatct gttcaacctc ctgcttgaca |
| 40561 | gatatgaaaa cagtgctctc atgtaattta tgtaatttac atttttacat atttagcagt |
| 40621 | acacctttgt gctatgttat attgtttgag aactaatctg tttttctgat ggctggaagt |
| 40681 | ctaagtgaaa agattataca tgaaaataat tcatgtttat tatcactagg agaaagtaag |
| 40741 | acaatttagt ttgattcaca agcatttgaa tccctattgt gtcagacatt gtattggaaa |
| 40801 | ctggggatac aaggaagata taacctcatc tctcagagtt ccctagttat cccatacata |
| 40861 | tatacaatta ttatttgtca attaaatttt ttttaaaaat ttagaaagga gtttccaagt |
| 40921 | aagaaaaatc atagcgaaat ggtataattg gtataataac agttaattag ctatagtatg |
| 40981 | aaaatacaga ggaagatcta actaagttgg ggattattaa agaaaattta aattagctgg |
| 41041 | gtgtggcggc agacgcctgt aaccccagct acttgggagg ctgaggcagg agaattgctt |
| 41101 | gaactcaggt ggtggaggtt gcagtgagat gagatcactg ggtaacagaa caagactccg |
| 41161 | tctcaaataa aataaaataa aaaataaaga aaattggaca gagatgtggc atttgaactg |
| 41221 | tctttcagat taagaagaaa gttactagtt agccaattca aatataagat ttacaaacat |
| 41281 | agagtacagc atgagcaaag gtgtggaggt acaaaaagaa taatgttttt gaggggagaa |
| 41341 | tagcaagtac tctgggggtg gccaaagttt gagttgtgtg gttttggtgg tggatggaga |
| 41401 | tgcaggaaga ttaggaagta gattgagatc agattgagaa aggtcttcca gaccagatga |
| 41461 | aaaattttgc catatttagg caatagggag ccccagaaat tgttcaacca tcatcagctt |
| 41521 | tacatttcac tatggtaatt tggcttcata agaagcatgg gttggagatt gataagagca |
| 41581 | gactccaagg ggccaattca ttaaactcaa ctgccaataa tgcaagagac aatgaaagcc |
| 41641 | agaatgaaca cagtgaccat ggatgccaga ggcatttcta ggatttttgt agaggactta |
| 41701 | agagaccatt tgggagggga ggaaggcaga agataagggg aaagaaggag ttgatatctt |
| 41761 | tgatgactga tggcactttt gctaagatga agaataaatg gtagcaagaa taaattgact |
| 41821 | gggcgcagtg gctcatgcct gtaatccaac cattttggga ggctgagatg ggtggatcac |
| 41881 | ctgaggccag gagctcgaga ccagcctggc caacatggtg aaaccccatc tctactaaaa |
| 41941 | ataaaaaaaa ttagcbgggc atggtggcgg gtgcctgtaa tcccagctat tcgggaggct |
| 42001 | gaggcaggag aatcgcttga acctcgaagg tggaggctgc agtgaacgag atcaagttat |
| 42061 | tgcattccag cctggacaac aggagcaaaa ctctggctca aaaaaaaaaa aaaaaaaaaa |
| 42121 | aaagtataaa ttgggggagg ggaggagata taggattcct attggaatgt attgagtttg |
| 42181 | agattccagc ctggacatat ggcactcttg aattgtttcc taggcgatca tatcactgtc |
| 42241 | ttgatgatat ctttaatcac acatatcact tgtggggaaa gaagaaactt caagagtttt |
| 42301 | acacaccaat cctttacttt gccatctaga caaattatat ttttaccaat gccatttttt |
| 42361 | gttgttattt tgtgggttct gcttaaaact tcccctatat ttttctaatt atggtaacct |
| 42421 | agggaaaaca tcaaattatg ttgataaaat tggctacaat ctggctctaa tgataatagc |
| 42481 | ttagaatcat gaagcagcaa tattttatcc tgagagcaaa caaactttat cctgaacaca |
| 42541 | tgtgttcttt tgccttattc atctaatatt acttgaaact tttatttttc cactttgaat |
| 42601 | agtagattct gccaagtgtt ttaaaagctt ttctcttttt aggggaggag gggaacagct |
| 42661 | tttctccctt ttgtctgata actgaggagt gaagatttaa ttggtgagaa aaatatacac |
| 42721 | actataaacc tcagaatgtt cccactgctt ttaatgtttg atgtgcctga cagattaccc |
| 42781 | tttcaaaaca gataatagga taggtggtgc tggaaaaaaa cttttccatc caagacaaaa |
| 42841 | ccacatggac attgaaaaga actggcattt gcctagacat ttagtggggg acatgtcctt |
| 42901 | agggcacaga ttccatagtg aaaggctctt ctcccccttc cacacactcc agctctctct |
| 42961 | agaaggtata atggaaagaa ctctgagcaa gaatcaggaa gttggccttt gttcctgact |
| 43021 | gtgccacttt gtggaactgg gcagaactta cctcattctg tgctttagca gcttcatttg |
| 43081 | caaaaattaa tgattacctc aaatgatttc tgattttagc caaaattaaa tacagtagtt |
| 43141 | ttaaaacaac tgctacatat taactactta cgtatgtaat tttgcactca aaataaatta |
| 43201 | tattatggaa aagaaactca acctatgtta accatccctg tactgatgag gaaactaaga |
| 43261 | cttaggaaaa agaagtgatt tactcagtat tgggcagtta gtgagcattt ttgagccaag |
| 43321 | gtcttacttt aaccagagac ctttgcataa tattgtactg ttactaaaca ctttctatta |
| 43381 | tttgactaga ggaacacaac atattgatgc atttgaaaca ataaaataat aaatattttt |
| 43441 | ccttaccctc ggcagaaaca ggatgatgag aaacattgac taaaaattta atcaagcctt |
| 43501 | aaggacaaat agtatcacag ttttgaatag gagattgggt accttggaag taattatgaa |
| 43561 | gaggtgtttt aactcagcag taggctcttt cttattcctg ctaggaatga agaagcacaa |
| 43621 | attgaaagtg aagaaggagg aaataaaaca ctgactctat gtgtaagaaa ggtgacttgc |
| 43681 | aaattatgag gcagttagga aaatgtttat tttttcttat tttataggct gtatatattt |
| 43741 | gagaattaat aattgtcctt tggagggtgt gtgtgtatgt gtatgttgga gctttgtgtg |
| 43801 | tgttttagtc cattttccta ctgctatgtg aagaaatgcc cgagacggta atttataaag |
| 43861 | aaaaagaggt ttaacggact caccgttcca catggctgtg gaggcctcac aatcatggtg |
| 43921 | gaaggtgaag gaggagcaaa ggcacatctt acatggtggc aggcaagagc gtgtgtgcag |
| 43981 | gggaattgcc ctttataaaa ccatcggatc tcacgagact tattcactat cacaaggaca |
| 44041 | gcatgggaaa aacttatccc catgattcaa ttacctccca ccagctcccc tcaagacaca |
| 44101 | tggggattat gggagctaca attcaagatg agatttgggt ggggacacag ccaaactgta |
| 44161 | tcagtatgcc tgtgtgtatg catgtgcacg tatataaatt ttttcactag tgactgatgg |
| 44221 | accatctata aaataaacaa aagagaagag acagggtctc actttgcctc ctaggctgga |
| 44281 | atacaatggc atgaccatag ctcattgcag cctctaattc ctgggctcaa gtgatcctcc |
| 44341 | cttctgagta gcbgggatta caggcacgtg ccattatgcc caactaatgt tttgtttgtt |
| 44401 | tgttgtggag atgggagatg ggagtactgc tatgttactc aggctggtct tgaactcact |
| 44461 | cctggcctta agtgatcctc tggctttagc atcctaaacc actacaatct taaaaaggac |
| 44521 | cagtttcctg gattacctct ctccctccat catccactat ctgccgtcaa ctctatagag |
| 44581 | tcaagggcta tccctgcctt ttcaatagaa gccctcaaga agtcactcca tagggactat |
| 44641 | cttgggcttt gggaagtgta cagggttggg gtgggtggga aggtgaggag gaaaggccac |
| 44701 | tgtttcaggt ggggttggag ggagatggaa aatggaccac agaggttcag agaacaggcc |
| 44761 | ctttgtggaa agggggagac caaataatgc tgcgttgtga aaggtgaaac aggctgcaaa |
| 44821 | cagaaggagc ccacaggaag ccctgctctg agcagctgaa cattctgggg tcctcagact |
| 44881 | ccgataaaaa agcacattta gcctacagct ttatgccctt cctcctaccc ttgtggactc |
| 44941 | tgccacagac atgccctcca gattgattct atccacttgt ctctagtcta ctgctactat |
| 45001 | tttagtctaa ggccttctcc ccgctttcct gggttatggt cacagactcc tatctgagtt |
| 45061 | cccagcacca ggtgcccagt tgttttcatc tcttaacaca ttccacatat tatagcaaga |
| 45121 | gagatctttc tcaaatgcaa atcagacctt gttgcatccc aatctcaacc cctttagttg |
| 45181 | ctcttctctt tgccctcatg gtcaagttca agctctctag tgtgggggca gatgttcctt |
| 45241 | tataattttt ctgcttatgt ctcctgcctc aggcctcaca attctccacc tcacagatga |
| 45301 | tgtctcagcc atgatgagtt cctctcaaat cctttaggtt tcagagatgt catttcccct |
| 45361 | gggaacattt tcctgactcc tcgttccaag catatgtctg tctttatgtt cccatagcac |
| 45421 | ctgatcattg gatcatggta gggtttatca cggtctcgta tttgtctgct tacttgtctg |
| 45481 | tattcttcac ttctcaaata tgtcttcttt gttcatcact gtaactcctt tccccagaat |
| 45541 | gttctcagat atttacaccc atcttccccc tctgtgaagg gaataaatgg atatataaca |
| 45601 | ctgtattagt ccattctcat gccactatga agaaatatcc aagactgggt aatttataaa |
| 45661 | gaaaagaggt ttaaatgatt acagttctgc atggttgggg agaactcagg aaacttacaa |
| 45721 | ttatggcaga ggcacctctt cacggggtgg cgggagagag aataagtgca agtaggagaa |
| 45781 | atgccagatg cttctaaaac tatcagatct catgagactc actcactatc acaagaacag |
| 45841 | cacgggggaa accgcccccc atgatccgat tgcctctacc tggttccacc ctagacatgg |
| 45901 | tgattatggg aattaaaatc caaggtgaaa ttttggtggg gatacagagc caaaccatat |
| 45961 | taagcaccat cctcaaaata gccttccagc tgaagacagc taaggcactc atgtagtctt |
| 46021 | attttgtctc agacaaataa ttttaccttt gaatcattgt tcattaatct agattttaac |
| 46081 | ctgaggccct aaaaattgtt tatcatcttc tgggagatct tactcctaga tggtgtttta |
| 46141 | ttccacaggt taaatttaaa cttgggccaa atctccatcc ttgtggcctg tgagtactga |
| 46201 | atttgggact gaaagcctgg gactaactgg aagtgtggaa aaggaggagg gtgtggcgct |
| 46261 | gccctcaatg attttttttt ttttttagga tttctggtgg aaaaggagag acactcagga |
| 46321 | atccccagat aaacatggtt tagcttttgt agtcagatcc tttccccaaa tttactgctt |
| 46381 | ctttctccac cacttctcaa ccccccaaac tgaacttgcc tcttcaacac actcccttgt |
| 46441 | gccctataga cagtcacaag cttgacctat agagagcatg tatttcttga ggcaacgaag |
| 46501 | gtaggaaacc ctattctaaa atgtttctta cacttttggc tctgtctaaa ttctccattt |
| 46561 | ttcaattaaa atggaaacga gattagaatt ctattctagt tatgaatata atcagaacac |
| 46621 | aataaagcag aaagattgct tcaggctctt ctgttaacgt tacatttatt caacaactct |
| 46681 | ttatggggac ttgctatgag tgaggcacca ggctaggcac cgcagctaca aaggtgaaga |
| 46741 | gaacataggc tcttcttcaa agaaacttac actgtaagct ttatagttat gaaatcatga |
| 46801 | ttgcttacct taaatgttct tgacctacat tcttagatat tttgagtctt gagaaatttg |
| 46861 | tttatttttt tcattattat acaatgtcaa ttagctcagt ggaatccaag atgttcggct |
| 46921 | agccttctga tccaagatag tggaccaaac atttgtattt actcattgtg cccctccata |
| 46981 | cttctctgaa ataaaagtgt agatcttcaa aattaaacat atctataaag caaagggaac |
| 47041 | agaaggggac attatcagca gattagacat tccaaaaatt ttttagaaaa taaaagctaa |
| 47101 | attgaatagt aagacacgga attattagat gtgtttggaa aaccaacagc atgaacaaga |
| 47161 | taatccaaga aaaacaagtt gtcccttggt gaaaatggaa ggaatttgag aaatatagaa |
| 47221 | gtgaactaaa caataaatac agtcatccct tggtatctgt tggggactga ttccaggacc |
| 47281 | tccctcagat accaaaattt ggatgcccaa gtctctgata taaaatggta cagtatttgc |
| 47341 | atacagccta tgcacatcct actgtatact ttaaatcatc tttagattac ttataatacc |
| 47401 | taatacaatg taaatgcttt gtaaatactt gttatattgt attgtttagg gaataatgag |
| 47461 | aggaaaaaaa gtctgttcat gttcagtaca gatgcaactt tttttttttc cagaatcttt |
| 47521 | ttaacctatg gttggttgga atccacaaat gtagaacccg gctacagaga gctgactgta |
| 47581 | attaaaacca acagcttcta tttggtatac tgaaagagaa ttgggaaatc attaaatcca |
| 47641 | taaaataaga acaggatgga gtgaaaaaag ctggactctg tgacgaaatg gacatatgta |
| 47701 | tggataagaa tcaggaaagt tttcataaag gaagtgccaa actgagctag ttggccaaat |
| 47761 | gaactattct ccaggtggat ggggcagggc atttaggcag aggaagcaac aaagcacaga |
| 47821 | gtgaagccca ggaaactaca ggtagctgca gggagatgac ggggctacag tgtgcatgga |
| 47881 | gagaggccaa catggtgagg taggcaggag gagctcctga agggctccaa cctgggaagc |
| 47941 | caggctaaag agatggcatt ctatcttgtc agtaataaga agcattgaca ttttgtagag |
| 48001 | atgatgaatt gaggaaggga tttatgtggt catttgtggt tctggctgat ccccctgagt |
| 48061 | gctgtctgct agaaggattc caagggttaa gtcctgaggc gtgaagaaga accgatctgt |
| 48121 | aagatagctt aactaaggtg gtagcattgg gataagggtg tggggtgtgt gtgtgagggg |
| 48181 | gatacaaagt tattaaggtg gtagaattga tggcaacatt catcttgaca gagaatacag |
| 48241 | gatgaggagc agatttcaga ggaatataat gagccctttt ttagatcttt gtgaatgttc |
| 48301 | agaaatggca gcbggtgttg cataattgca aaattagggc ttagttagtt attggcttct |
| 48361 | gagaacgtaa ctgcagtttt tttctttccc tctgccccac tctttgaaag ggggtggggc |
| 48421 | tcaaagagta gtgtgtgtgt gtgtgtgtgt gtgtgtgtgt gtgtggtaaa tacttatata |
| 48481 | caaatttctt tagatttttc tttatctcct tcactccttt tcaacatttt ggctggttgt |
| 48541 | tctgatatta ccaaggtaat ccacaattat ggcaaatatt caaacaaaac aaattataga |
| 48601 | aagtccagaa aaaaaaactc tgaaatacct tttccagaga ccaccatcat gaacagtttg |
| 48661 | gtctacagaa ttacaaatct tttgaagtat atagtatatt cttttatgtg gccattcatt |
| 48721 | atattttatg ggatcttagt attattgctt ttaaattcat ttcttatatt ttaaatttga |
| 48781 | aatacttcaa aatatgcaga aaagtagaga aaataacatt aacaccatat acttaccatg |
| 48841 | tagatttaag agaaatctac actttgccaa atttcctttt tttttttttt tttttttttt |
| 48901 | ttttttgaga cagagtcttg ctctgtcacc caggctggag tgcagtggtg cgatctcggc |
| 48961 | ttactgcaac ctctgcctct tgggttcaag caattctcct gcctcagcct cctgagtagc |
| 49021 | tgggattaca ggtactcacc accatgcttg gctaattttt gtatttttag tagagatggg |
| 49081 | gttttgccat gttggccagg ctggtctcga actcctgacc tcaaatgatc cacctgcctc |
| 49141 | ggcctcccaa agtgctggga ttacaggcat gagccaccgc acccgaccaa atctccattt |
| 49201 | ttgtaaataa tttttcagat atagaaaact acccaattat ggcbgggcat ggtggctcat |
| 49261 | ggctataatc ccagcacatt gggaggccaa ggcaggcgga tcatttgagg tcaggagttc |
| 49321 | gagaccagcc tggccaacat ggtgaaatcc cgcctctaca aaaaatataa aaattagccg |
| 49381 | ggcatggtgg caggtgcctg tagtcccagc tacttgggag gctgagggag gaaaatcact |
| 49441 | tgaacctggg aggcggaggt tgcagtgagc tgagatagca ccattgcact ccagactggg |
| 49501 | tgacagaaca agactctgtc tcaaaaaaaa gaaaagaaaa gtaagaaaac tacctgatta |
| 49561 | ctattacttc tctctttctc tctctctctc tgtgttctag aggtttgcag tatcctgaag |
| 49621 | ctggtgtgaa tcaacctcac gctttttttt tttttctttt tttttgaggt ggagtctcgc |
| 49681 | tctgtcgccc aggctggagt gcaatggtgc aatcttggct cactgcacct ccacctcctg |
| 49741 | ggttcaagtg attctcctgc ctcagcctcc cgagtggctg tgattacagg catacaccgc |
| 49801 | catgcccagc taattttttg tatttttagt aaagatgggg tttcaccatg ttggccaggc |
| 49861 | tggtctacaa atgtatctat ccataagcaa taccgtatat agtaatattt ttattattct |
| 49921 | attatttcta ttgaatgttt atattcatag tatcacactg tacacatcct tttgcaattt |
| 49981 | gctttcttta gaagcaacaa tatatttttg agatttatca tgttgctttg tgtagattgc |
| 50041 | ttactttttt tttttgatgg agtttccctc ttgttgctta ggctggagtg caatggcgta |
| 50101 | atctcggctc accgcaacct ctgcctcctg gattcaagag atcctcctgc ctcagcctct |
| 50161 | tgagtagctg ggattacagg catgtactac cacactcggc taattttgta tttttagtag |
| 50221 | agatgggctt tctccatgtt ggtcaagctg gtctcgaact cccaacctca ggtgatctgc |
| 50281 | ccgcctgggc ctcccaaagt gctgggatta caggtgtgag ccactgtgct tggccaatta |
| 50341 | tttacttttt aatttatttt atgatacaca atttgtctct ttatctcact ttattttttg |
| 50401 | attttttttt ttttaaacca ggatctcact tggtcaccca ggctggagtg cagtggcgtg |
| 50461 | atctcggctc actgcatccc ctgcctccca ggtgcaagca attctcctgc cttggcctcc |
| 50521 | tgagtagcta ggactatagg gtgtaccacc acatctggtt aatttttgta ttttagtaga |
| 50581 | gatggtattt tgtcatattg gccaggctgg tcttgaactg ctggcttcga gcaatttgcc |
| 50641 | cgccttggcc tccaaactct ctttattttt tagatattgt taacggtact atgggcagta |
| 50701 | tccttgaaca tctctctttg tataaatgtg aaagacattc atagacatgg gactaaagga |
| 50761 | ggtgcccata gtcaaccatg ctaggcattg acaaatggct tcctaaaggg gtaatacctg |
| 50821 | tgtacattgc cactagcaat ttgtacactt tcttgattcc tttcactctt gtcaaaactt |
| 50881 | actctttttt aaatttaaaa ttattggcca ggccatggtg gttcatgact gtaatctccc |
| 50941 | agcactttgg gaggctgagg caggtggatc acctgaaatc aggagtttga gaccagcctg |
| 51001 | gccaaacatg gtgaaaccct gtctctacta aaagtagaaa aattaactgg gcatggtggt |
| 51061 | gggtgcttgt aataccagat actcgggagg ctgaggcagg agaatcgttt gaacctggga |
| 51121 | ggtggaggct gtggtaagcc aagatcacac cattgcactc caacacaggg gacagagtga |
| 51181 | gactctgtct caaaaataaa ataaaataaa ataattgtca atattgttgg ttatgaaatg |
| 51241 | ctatctcatt attgtttaaa gaggcatttc ccttgataac tagtgaggtt gaattttgtt |
| 51301 | ttccagatgt gcatgagcca ttggtttttt tctgttatgc attttctatt catatacttt |
| 51361 | gaccacatga tatgtgcctg gtcataaccc atcaaactct tctttctctc agatacttca |
| 51421 | ctttatggaa atctttttca cgtttatccc tcaaacccag aattttgagt gtcatacctg |
| 51481 | ttctaacata aatttgagga aaatgaaggc taattcagaa taaagaacta tataagcaga |
| 51541 | tggacagtgt gttctcctct aaataggggt gctaggataa gaggcggtag gataaatgca |
| 51601 | cagcctcaaa atccaccacc cactaaagac ctcacagtac atgatgacat tagcatgctg |
| 51661 | tgccagcatt tctacctacc acgatgacct ggaatgaagg acaaaagggg caattgttaa |
| 51721 | gtcctgtgcg taaactttgg aagagaacat accttgcttt aaaccattaa gggggcaggc |
| 51781 | aatagattcc tgtacagggt ggcttagcca aaagtggctg gtcttgttct catttatatg |
| 51841 | gtttataatg attctaaatc attctcggag aaaaacagac tcatttcatc tccctttaat |
| 51901 | taggatttct gttactgcaa tattttagtg gcatttatgt caaatgtttt cttttgagaa |
| 51961 | cttctccctt ttactgtctt ttttctttta ttttaaactt ttattttagg ttagggggta |
| 52021 | catgtgaaaa ttggttacat aggtaaactc atgtcaagag ggtttgttgt acggatgatt |
| 52081 | catcacccag ggattaagct cagtactcaa tagttatctt ttctgctcct ctccctcctc |
| 52141 | ctactttcca ccctcaagta gaccccagag tctgttgttt tcttctttgt gttcataagt |
| 52201 | tctcataatt tagctcccac ttatatgtga gaacacgcag tatatgattt tctgttcctg |
| 52261 | cgttagtttg ctgatgataa tagcctccag ctccatccat gttcctgcaa aagccatgat |
| 52321 | cttgttcctt ttaatttact ttttgctatt ctgattagag agtggccatg ctccatgctc |
| 52381 | catatggata ttctttggaa acaaagcatt tttgaaatgg taggattggg gctagtaaaa |
| 52441 | gagaacagaa agaattgtga acagggtctc ctccagtaca gatggttaaa gaacacaaat |
| 52501 | agggaagtgt gatgagtgac ctccatttaa ttgggcgccc aagctcttgg ctgctccagc |
| 52561 | atgggttgcc atcctgactg atcccttggc tcagaaacag atatggtgct atcaggaaag |
| 52621 | caaggccaat atctgaatat tgcagcagag actgctccca tttcagaaag caacccagat |
| 52681 | taaagttgat gattcagttt caataaatgc ttcctttttt ataaaaggca tttaacatca |
| 52741 | ctctttcccc tggtaatatg gaattagctt ttgggatcaa ctggaaaatg tcagagggcg |
| 52801 | attgtaaaag gccacctgct aaccacagga agcbgggctg atacagaatg ccaggttaac |
| 52861 | ttaaggtcga taatggttac ttccttgtca ctttttacat gcatccttgc tctgcagtgg |
| 52921 | gggtcataag aagtaagatg catgcaaggt gaatttctag aagtcagctt ctctgagaag |
| 52981 | tattctctga ccatacattc ctactagtcc ccacatacgc tgctgatctc cccatcttca |
| 53041 | cccccattgt tgaaattgct ccctcattgt aactgttcac tcatctgtct ccaccataaa |
| 53101 | gcttaggctc ctagaaagtc aggaccagag ctagtacagt gccccatgtt cttatggaat |
| 53161 | gattttagtg ccttgagttt ggtgtggtga ccagagctgg tggccaactg tcgtgaggcc |
| 53221 | ccagtccttc tttttgtcat gctttccctc ccagcccctc cttgaccact ctttcacctc |
| 53281 | catctttcta cttatataga gacaaataat aagatgcttg tacaaagggt gtacttgtat |
| 53341 | tatcccattt gtgttgatat tccacaacag caaattaggt ttttttgttt gtttgttttt |
| 53401 | tgatggagtc ttgctctgtt gcccaggctg gagtgcaatg cgtgatctcg gctcactgca |
| 53461 | acctctgcct cccgggttca agtgattctc ctgcctcagc ctcccgagta gctaggacta |
| 53521 | caggcaccca ccaccaagcc cggctaattt ttgtattttt agtactgacg gggtttcacc |
| 53581 | atattggcca ggcbggtctt gaactcctga cctcaggtga tctgcccgcc tcggcctccc |
| 53641 | aaagtgctgg gattacacgt gtgagccact gtgcctggcc tgataaggtt taaagattaa |
| 53701 | ataccatgtc gttaaatttg caaatctctc gaataggaaa atcaggaaca atgacaacat |
| 53761 | tcggagcttc ccagaaggta acaactatgg ggagttttgt gggcatctga gagaagcaca |
| 53821 | cacacagatg gtttgccttt cattggtcca ttcttgccca gaaggcgaca ctaactgaaa |
| 53881 | caaagaagcc tcatatgaga aggacatggg gtcacagcag cttttggggt ggccctgggt |
| 53941 | ccttgatctg gtctttctct ggctcatttt gtattgctca ctttgttact tcacaatcca |
| 54001 | gggcttgtga catttttctt ctctggagtg gaaatgacag agctgcagaa cacaagtttc |
| 54061 | tgattggttc ctatgagtca ggaaagaggg ccagttgaaa ctgggctcac acacacacac |
| 54121 | acacacaaac taggctcaca tgtgttttcc catcagctaa acaagttttc agaagtacag |
| 54181 | aaataactcg ttggggaagg ggacccagga caatgacaat gtttttgatg gtaatgtctg |
| 54241 | cagaagtaag gcaggtttcc tctgtgcctg aaaagatggc accatttgag gagatggcat |
| 54301 | actcgtggtg gaatcttcat taagatgtca aactaagctg taggaaatca actctagaaa |
| 54361 | acagctctaa tgtgagaaaa catatatcca gttttataga gcaagagctg tgatcatggt |
| 54421 | gacatttaac aactgtatta gttggccaag agggtaaaaa tatacttgac ctcttagagc |
| 54481 | accataaata ctctcagctt gtgggccaac tgtgtcatgg tcttttttct agcccctcct |
| 54541 | ctttctccct tccttcttgt ctctctcctc ctctctcttc cctccccttt ctcctttctg |
| 54601 | accctctttt ctcttctcta tcttctcgaa ccttcccttc ctcctctcct ttaggtgctc |
| 54661 | agcacctctt tgattcaata aacatttacg gagctccttc ttgtgccaac gactgtacac |
| 54721 | agagcbggga tgtcaagatg aatcagttac atttcctgcc caatgccaag ggatggcttg |
| 54781 | atgggttttg accagcccct ccctggactc ccaatttctt tttttttttt ttcttttttt |
| 54841 | tttttagacg gagtcttgct ctgttgccag gctggagtgc agtggcacga tctcggctca |
| 54901 | ctgcagtctc tgtctccctc ccaacttcaa gcgattctcc tgctcagcct ctcgagtatc |
| 54961 | tgggattgca ggcatgcgcc accacaccca gctaattttt ttgtattttt agtagagaca |
| 55021 | gggtttcacc atgttggcca gggtggtctg gatctcctga cctcgtgatc cacccgcctt |
| 55081 | ggcttcccaa agtgctggga atacagtcat gagccaccgc gcccggtgcc ctggactccc |
| 55141 | aatttctacc tcctggaacg tgctttactc accagccttg gtgttattct gcaatatggc |
| 55201 | caactgggat ttttttcaat cttacagaaa ttattctcgg aaatgtttgc tatttttttt |
| 55261 | taatgaagct acagcataca atttaaggat aaataatgta cttactctgc agctattgtt |
| 55321 | tcaagtcaaa tccgctgatt actcctccaa aattcttttc ttaataaaat ctctgaccct |
| 55381 | ttcctcttcc tgttagaact tctggagcta ccattgttcc tcctcttggg ctgctcataa |
| 55441 | cctaggggag tcacagtgaa attacttgct ttaaaattct ttaatcactc tgcgagttgt |
| 55501 | taggaaaagc tgacacatct gatttggaaa ctcccactcc agtggactgt tgtgcctcag |
| 55561 | cacacaggga cgggtggagc aaaggaacaa gaagccaaaa actctcaggg cacttactat |
| 55621 | gattgtgtaa aagagagact catcaattaa cacaggtact ggcaagtgca cttcataatg |
| 55681 | cctccatttc tagacagatc aaaaacagtg tgccctgtca ataaatgctg agtttctgta |
| 55741 | gcagcctgat tgagatcagc taacctttag gccaaaagcg ggattcttcc ttgctgaagc |
| 55801 | ttttggcaaa gcatattgag cttcatgcca gggattagat taactcttgc aaaactggtt |
| 55861 | caccaggaac aatgactcag tgtttttttt ttagtatccg gcatattgtg tggagcagct |
| 55921 | ataaatatat ttacaagctg gagtttcaag ggaggggagt aatagcttgc tgaaaaacat |
| 55981 | gccaactgag ttatgggtct tcacgcagcc agcatgctgt gttttgggct ctgcctcttg |
| 56041 | taatttttag aaattaatta gtttattctt tatttatatt tatttattta tttattttca |
| 56101 | gatgggatct tactctgttg cccaggctgg agtgcagtgg tgcgatctcg gctctctgca |
| 56161 | acctccgcct cccaggttca agtgattctc ctgcctcggc ctcctgagta gctgggattt |
| 56221 | caggtatgcc tcaccatgcc tggctgattt ttgtattttt agtagaggca gggtttcacc |
| 56281 | atgttggtca ggctggactt gaactcctga cctcaagtga tccacctaag ggtactggga |
| 56341 | ttacaggcat gaaccactgc acccggcctg ttggttgtaa ttttattttc ctcctcaagg |
| 56401 | actttgagca cattatcttc tctttaataa cttgtttcca ccctcctttg tgcacgcgcg |
| 56461 | cacgcgtgtg tgtgtgtgtg tgtgtgtgtg tgtgtgtgtg tgtgtatttt ctgatctccc |
| 56521 | tacgggggca gagagtctca agcaggtatg tccatgttat taacatactg tctgcttatt |
| 56581 | aactaactgc accaatgatg aaagccaact gacaaacaag tgatgctctt atccctcctt |
| 56641 | cgcaagcaat gaatctgaaa gccaagagaa gttttccttt caacaggaag tgttttggag |
| 56701 | tccgaacaac tctgcattta gacagtgcag cttttagtta cagatcttgg caatggggca |
| 56761 | ggcagaggga gcggcaacta cagactttgc cgtaggactg ttttctatgg agtgtgtgtg |
| 56821 | ctctgacgag ctagtgacag gagggaggtt acaaaagcat ttactgaaat aaggatttct |
| 56881 | aatagaggag ctttttaaaa gtattaaata tgctaatgtg cctagggcat agtcaacaaa |
| 56941 | gagttataat acatgtcatt tttcatattt atcaaggaaa ccatgtcacc cgcagccttt |
| 57001 | ctcaatttct ggcattctga gaaacacact gtagtggtgc ctgagggtct ggggaaagaa |
| 57061 | gaatagaatg aatggggcat ctcagaatcg taaactcccc cagttgtagt tcctttcgat |
| 57121 | gttggtcgtt ggatgaattt ccagtttcta ctattgtggt tggattgggg agagtagctc |
| 57181 | aaattgggaa ttgctttttg aggatctagg gcttctggaa ttctcccagt ttctttttac |
| 57241 | ctctttatgc tatcttgtct actttgaaaa atcagtagca tccatttgat tgcagagctg |
| 57301 | aatctttcta tacagtgagg agttgtaatc acattactta taacctgctt atttaacttt |
| 57361 | ttaaaggtgc aagtcattgt aacctaagta tttactcaca ctgatcctta atatactccc |
| 57421 | ctttcttatc tgcaagcttc tttctctggt ttctcctaac tctgttactg ctcatgagtt |
| 57481 | taaaactttg tcttctcagt tcctggcttc acctgtttgg tctctgctct ccaggactct |
| 57541 | gccttggaaa tctgactcac acttctatat gtccctgggg ctcaacttcg ggcctggcgc |
| 57601 | cagaatcacc caggctccaa gcaagccttg tagactccac tcagtccttt gactcaagga |
| 57661 | aatctccact tctaaatggc tctccggttg attctgatgc cagtccaggg ttgagaacca |
| 57721 | ccgctttagc ttctagacac acagccccct cccctttttc ctggagcccc tcttgggtca |
| 57781 | tgattctatt ttccctattt ctgtactcag aagggataaa aatggaagtg tatgtgaagt |
| 57841 | tgctactacg tcccaggtgc tttaaatatg ttattttatt caatcttcac aacagtctta |
| 57901 | tgcagtctct atttttattt taagaatgag gaaaacaata cttgggtgca gtggcttatg |
| 57961 | tttgtaatcc cagcactttg ggaggccaag gtgggtagat cacttgagcc caggagttca |
| 58021 | agaccagcct gggcaacatg gcataaccct gtctctacca acaatacgaa aattagccag |
| 58081 | tctcataacc tggtctataa ataaataaat agataaaaat ttaaaaataa aataatttaa |
| 58141 | aaagaaaaga atgaggaaaa caagattcag agagccttgg tcatcttccc aaaggtgcac |
| 58201 | aattaagaaa tggctaagca ggggttctga ctcagatctg tcagcatcta cagctttgct |
| 58261 | atttaaaaga gcgtggtccg tggactagca gaatcagtgt cacctgggat cttggtagaa |
| 58321 | acacaaatac ttgagtgcca tctcagacgt actgaatcag tttcttcagg ggtagggtcc |
| 58381 | aggaatatgc attttatgaa ggcctctaaa tgtttctcaa agtttaagaa gcatggggaa |
| 58441 | ctatatcttt tcctactata tagtcttatc tctttccttg tttctttact ccaaagtatt |
| 58501 | gccttatttc tttcccagaa acctcactat cagctcactg tgcagcctta ctcatatctt |
| 58561 | cctcaaccag cagttttatt acacacaccc ttctcatttc ctcacattgc tcatggtatc |
| 58621 | tgactgagta cagggatcca acctggcttt gggttttgca ctgtcttgcc tcagtctctt |
| 58681 | aatttcttcc tatgtacatt ttaagatatt gaagacacta acttccaaac ctcttctcta |
| 58741 | aatctgacag tgaagcagtg tgtgtgtgtg tgtgtgtgtg tgcatgccag tgcatacacc |
| 58801 | acacatacaa gcacatacac agagttaagt ttggtcacat ttacttcatt aattgatata |
| 58861 | taccacaact gagaaaaact tactggcatt ctgctccaag cttgaattcc cagacgaaaa |
| 58921 | acaaacaagc aaacaaacaa ctcctcctta cacaaaaata tctagtggcc ttggaaaatt |
| 58981 | ccatttaaca ttaaattgtg tctcttcaaa gtgaagcagg gtgggcagat ttttgaacat |
| 59041 | gaatctactg ataacacaac acctcttcaa aactagaatc tcattcaggg ttaaatgatg |
| 59101 | tccaagtcaa acctgagatg ctagcatgat gccccatcct cagtcagagg tttcagggtg |
| 59161 | gcaaacaact tttgctcact ctcctctgcc tctctctata tttgcagaaa gtccagcttt |
| 59221 | cactgcaaaa gttcgataac tctgtaaaac agtttgatcc taacttcaaa agaattttta |
| 59281 | tgctatttgg aatacataaa aaatgaattt attccaatac tttagcccca taaataatgc |
| 59341 | ataatgattg gtgaaagatt agcattttgg agttcaagta cttaaagctt tcttggatgg |
| 59401 | ataatgcatc acccttaact agcataaggg gaaatagaag gttgacaagg aaaacttaat |
| 59461 | ttagggttct ttatttattt ttctctagtt atgtttacct tcccaggttg ttttattaat |
| 59521 | gccacttttc acaaggatat ttcatgaata aatatcaggt tgtatctctg gccatctgtg |
| 59581 | atgatcacgt ttttattttt aatgagttgc tcatggatta tatcctaact ggaattgcag |
| 59641 | tcagtatatt gagatgcatt gactccattt ctgtctttca ttcttctttc tcaatgtatt |
| 59701 | ctcactggat tagcactctt agccctgagg actcttgggc cagttttatt actaaaagaa |
| 59761 | gaattacagt tttgctacac agtgaccttg caagaagtat cagcacagct caatttcaaa |
| 59821 | ttctcctcaa atatcaaata tgttcttggt atattttgct tagcagttta ttgccttctc |
| 59881 | atctccactg tttttcctga gtaacagctc agaaatctat aaataaaact taataatggg |
| 59941 | gagtgactca aaagtctggt tatttttatt agtaaactat tttaggggtg aacacttcac |
| 60001 | attttacatt ctcatgctcc acagtgctga aaatagtctg aatttttttt gtgtgttagg |
| 60061 | ttgatgagaa tatttttgag atgactagca gaaaacagaa tcattaaaac agtgctatca |
| 60121 | gttttcatgt tgaataactt tacaggaaat gatagcatct ttttttcttt ttttgagacg |
| 60181 | gagtttcgct cttgttgctc aggatgaagt gcaatggcgc gatctcggct caccgcaacc |
| 60241 | tccgcctcct gggttcaagc gattcttctg cttcagcctc ccgagtagct gggattacag |
| 60301 | gcatgcgcca ccatgcctgg ctaattttgt acttttagtg gagatggggt ttctccatct |
| 60361 | tggtcaggct ggtctctaat tcccgacctc aggtgatcca cccgcctcag tctcccaaag |
| 60421 | tgctgggatt acaggcgtga gccactgtgc tcagccgata gcatcttaac aataaacatg |
| 60481 | caaagatttg taaatgttgg caaacagcaa gaaaaacata tttagaaaat tgtgctatgc |
| 60541 | tttaatgtcc tgtctgtacg ataatttact tagctcccta ccttcccccc agcccaccag |
| 60601 | aatgtttaaa ctgaaattta aagcacagtt gatcttggct tggataggca gcctgcatat |
| 60661 | taactgagga tttagttagt atccaggagc agatctactt ctcaaagtga gattcacata |
| 60721 | gcccccttgg aagctttcaa atagattgac tagcctttta aaattcctct tctccccttt |
| 60781 | cttattgccc ccgccttgtc tagctgtatg ctaacttgct gcatagaatc ctgaatttct |
| 60841 | ggatttctac atttctagct caatgaatgg ggtaaatcaa aacttaataa cactcttgat |
| 60901 | tttgaaaagc ttcagtgctt catgtaagac tttttgagaa aagatcattt tcatttattt |
| 60961 | ctgttttatt tctagcttaa tgattaaatg taagcbgttc aatacctttt tcaatatccc |
| 61021 | acttgttaaa cccacactaa acctcagaaa tattgagtca gtgatctaaa tttccattca |
| 61081 | aaatttccac aaagcagaag cataaatgtt ttcattgtca ttacaaataa acagacaggg |
| 61141 | ctgggcgcag tggttcatgc ctgtaatccc agcagtttgg gaagctgagg taggcggatc |
| 61201 | acttgaggtc aggagtttga gaccagcctg gccaacatgg tgaaacactg tctctactaa |
| 61261 | aaatacaaaa aaattagctg gattggtggt gtgtgcctgt gatcccagct actcaggagg |
| 61321 | ctgaggtagg agaatcactt gaacctggga ggcggaggtt gcaatgagct gagattgtgc |
| 61381 | cactgcactc cagcctgggt gacagagtga gacttcatct caaaaaacaa aaacaaaaac |
| 61441 | aaaaacaacc ccttcccctc cacgaaagaa aaccccaaat aaacagacaa atgaaagcaa |
| 61501 | tgaaaacagg aaaataattt tgacacctac ttctgggatg ttatgttatg ctttgcattg |
| 61561 | gttctattta ttagacatgt tcccatcttt attatttaag agcagataaa ataacctcac |
| 61621 | tacattaatc tcttttgctc tgcctctttc catatgattc ttcactagca gttcccaaaa |
| 61681 | caaaaaaatt gtgcaccaaa atcccaactg agactcaatt gtttgctgac tcaacaactc |
| 61741 | taatgagaaa tcttatacaa gagcttttgt ttcaaaagga agtgccagga aaattttgct |
| 61801 | tccctcaagc aatggcatgt tttctttttc tggacagtga gaaccatgta attaaacata |
| 61861 | ttttcctttg tgctttggct gtcaggaaac tcagagccaa acttgtagcc caagttttat |
| 61921 | ggctatgtaa gacattatgg cccatatatc tggactcttc ctttctcctt ggtattgatc |
| 61981 | ttcagtcttg gctttagttt acaaaatatg ccattataaa aaataacagt atctgtaggc |
| 62041 | tgtctgaaat tattatggaa aaagtagtgt acaaataaat gaataagtca attgtttgaa |
| 62101 | aaacaatgca tttgtatcag aaataacttt gtgagtggtc ttcaggttcc tagtctattt |
| 62161 | cattttgata tatatttctt gtgggtttac cacgtgccag acaaagatgt tcctggcagg |
| 62221 | ggggttaata atgttaacag tactcagtct ttctagtgcc ttacaggcta tagaaggaga |
| 62281 | aagaaataca aaacaaacta aaataggtat tcaattgcaa caaaagcaac ttgatgtagg |
| 62341 | ggtacagggg aaggaataat tcattctaca ggcctttaaa agataaagag gtatttcaaa |
| 62401 | ggcaaacaat ggagagaaga tcattctaag ctaaggaggt tgcatgaaga aagcataatg |
| 62461 | tcactacttt tttttttttt tttgagacag ggtctggctc tgttacccag gctgtagtgc |
| 62521 | agtgatgaga tcacaggtca ctgcaacctc caccttcagg gctcaaacca tccttccaca |
| 62581 | tcaacctccc aagtagctgg gactacagct gcatgccacc atacccagat aatttctttt |
| 62641 | attttttgtg gaaacagggt tttgccatgt tgctcaggct ggtcttgaac tcctgagctc |
| 62701 | aagtgatctg cccaccttgg cctcccaagg tgttgagatt acaggcatga gccaccgcac |
| 62761 | ccagcataat gtcatcaaat tgaagtgaga aatgggaatg tcactaccaa ccataagtaa |
| 62821 | taaacaatag attatcatta actttgctcc tagagaaaga caagtttatt tagaggtgag |
| 62881 | gcgagtagaa tttttttaag ggaacttctg ggtaatttaa caccatgatc cttctcgaat |
| 62941 | gccttacaac gtaaaaatgt agttctcctt tttcttttct tttctttttt tttttttttt |
| 63001 | ttgagacagg gtctcgcttc gttgcccagg ctggagtgca gtgacacaat cttggctcac |
| 63061 | tgcatcctcc gccgcctggg ttcaagtgat tctcctacgt cagcctcctg agtagctggg |
| 63121 | attacaggcg tgcactacca cgcctggcta atttttgaat ttttagtaga gatgaggttt |
| 63181 | tgccatgttg gccaggctgg tcttaaactc ctgacctcaa gtgatctgca cacctcgacc |
| 63241 | tcccaaagtg ctgggattac aggtgtaagc caccatgcct ggccattttt cttgataaag |
| 63301 | gggtgtcatt ggggcttctc ttactcctgt cactcatcat gggtgccatg tctttttttt |
| 63361 | ttttttttga gatggagtct tgctctgtcg cccaggctgg agtgcagtgg cgcgatcttg |
| 63421 | gctcactgca agctccacct cctgggttca cgccattctc cggccgcagc ctcctgagta |
| 63481 | gcbgggacca caggtgcctg ccaccacgcc ctgctaattt tttgtacttt tagtagagac |
| 63541 | ggggtttcag cgtgttagcc aggatggtct caatctcctg acctcgtgat ctgcccgcct |
| 63601 | cagcctccca aagtgctggg attacaggcg ggagccactg cgcctggcct gggtgccatg |
| 63661 | tcttaattga caaccacaat gaagagagga aggaaagagg aaagaataag acaatacaag |
| 63721 | aagaaaaggg aaggaaaagc caatgacaga cattaactgg attttcccac aaagagaggt |
| 63781 | catgaggatc aagagcatac agaagtttgc aaaataaaga gtttctctgg cttggagttg |
| 63841 | accaaaataa gagaaaggtg aagcgagtat aatgtacgga gacttctcct gggagaagtt |
| 63901 | gtcagtggag aagttattgg gtcatatagt gatggtgtgc agataggacc tggtggtgag |
| 63961 | ttgacgagga gccattgcca ttcagccatc aagaatcatg atgtgtgttg tgtaggaatg |
| 64021 | gagaggtgag tgtggagtgg atggggcgta gatggagaag caggaggatt cgtcatgcac |
| 64081 | cctgaggctt gccagtattg atgtcagact tcctccgacc gtgttgccac cctggactgt |
| 64141 | ttgactttgg tagctatttt aaaacagcaa gaagagctgc ttgagaagaa cctgggggag |
| 64201 | atttcacaac cagcagctgc atcgaatctc cctggatgtg tgcttttgga aacaatttac |
| 64261 | acaagggctg atttcaacat gcactaatat atacagttta gctggcattt tatatggaaa |
| 64321 | ctttaataaa atggttctaa ttagtgctta atttacatag attagagagt tctacaggcc |
| 64381 | cacacagtag ggcttgggga ttttatgtca tttgatggag atgccagttt gattcagata |
| 64441 | aagaaccaat tttagggaat actgctgaag tttgcccgac acaaatgaag gagttgtagc |
| 64501 | ttgcatttct tttacccact cttgttcagt ggcttgaact agctttctgt ctcttccaca |
| 64561 | agatttagga caagatggat ttctgtgtca gcatgtagta ggagagcttt gattttctag |
| 64621 | ttctgtcctg tgtggatctg tatgtagctc agattccccc acaatctagg tcagagaagc |
| 64681 | tgctagcctt ggtgcaaagt tttcaaaaag gcttctattc agagagatga ataaggaaaa |
| 64741 | actcacaccc tacgtgcgaa ggttgcttaa tgagaggtgt gagatggagc tcagttgcca |
| 64801 | cttatctctg tagttgggta ccgttgtgca gtctgtgcac tgcccagcca tttgcagtga |
| 64861 | ccccaaagag gatcatctgg tcatcttgcg gggagagact gtgagttcct gccatgtcac |
| 64921 | ttggtataac atgagaaaac tcaaaactct aggcagatct ttgcacctgc cctacttttc |
| 64981 | tacctgctct cacaatcttc ttgagggtga ccagagtcca agtaactaag tgtaattcaa |
| 65041 | tgtggtgaat gatatgattg ctacacagga gtatctatta aggctttcaa aatgtaaaga |
| 65101 | ggaagtgact cattcttcct ggtggtggtg gttgcatcaa ttggtgtagt ctattgcctc |
| 65161 | tctcccatca acaagggaca tttggcaaag tctggagtgt gtcaactgtg gggtggtggt |
| 65221 | actaccggca tctagtggat agaggtcagg gatgctgctg aacattttac aatgcccagg |
| 65281 | acagccctcc cacaataaat tatctggccc aaaatatcag tagtgctgcc attgagaaat |
| 65341 | ctggttatgt aacagataac tttttttttt tttttgacat ggagtttcac tcttgttgcc |
| 65401 | caggctggag tgcaatggct tgatctcgac tcactgcaac ctctgcctcc tgggttcaag |
| 65461 | tgattctcct gcctcagcct cctgagtagc tgggattaca ggcatgcgcc accacgccca |
| 65521 | gctaattttg tatttttaat agagacgagg ttttaccatg ttggccgggc tggtcttgaa |
| 65581 | ctcctgacct cagatgatcc acccacctcg gtctcccaaa gtgctgggat tacaggcatg |
| 65641 | agccactgtg cccagccaac agataacttt ttaaattctc agcbgtttaa cccaacaaat |
| 65701 | aattattttc ttcctcaagc tgtatgtcca gcatgggttg gcaacatagc cctgtttttc |
| 65761 | ctagtccctt caagaagtct attgatggaa gatgcatttt tatgtgggct tctaccacca |
| 65821 | ctatgagagg gggaaggaaa tttgttggat tgtaccctga attttaaaaa gcttctgccc |
| 65881 | agaattgaca tatattcttt ccaattgcat tttattggcc acaatccatc atatgaccat |
| 65941 | gcctgaattc agagggagta ggtggagaat acatttttaa tttttaattt tttttttttt |
| 66001 | tttgagacag agcctcactg tcttgcccag gctggagtgc agtggcgcca tctcggctca |
| 66061 | ctgcaacctt tgcttcccag gtttaagtga ttctcctgcc tcagcctcac aaatagccgg |
| 66121 | aattacaggc atgcaccccc atgcctggct aaggtttttc atatttttag tagaaatggg |
| 66181 | gtgtcaccat gttggccagg ctggtctcaa actcctggcc tcaagtgacc catcctcctc |
| 66241 | agccttccaa agtgctggga ttacaggtgt gagccattgt gcccagagag agtgcatttt |
| 66301 | tgtcttgtac ccagaaagag gagagagtga gaataatcat gaactaccct aatgactatc |
| 66361 | atgctgatgg taatggtggt tgtaggatgg tgtgagaatt taaagacggc ttcactcctg |
| 66421 | tcactttgct tacttactgc tgtataacaa actgccccaa acttcaggga tgaagctaac |
| 66481 | aaaaaaatca tttaatttgc tcatgaagtt gcaatttgga tagggctgtg tagaacagct |
| 66541 | cctctctata ccatggggca tcagttagga cagctcaaaa cctgggagta actcaaaggc |
| 66601 | tgggggctat aatcatctga atgcttgctc actcctgtgc tctcatgtca ggcagtggat |
| 66661 | gctggctgta gacagagacc tcagcbgtgg ctgtcagctt gaccacctac tcccagcctc |
| 66721 | tacatgtggc tgtttggctc ccttacagta tggtggctgg gttccaaggc agaaagtaaa |
| 66781 | tgctgccagt ttcttaaagt ctgtctgcaa attggcacag cattctttct accatattct |
| 66841 | attcatcaag gagttgtaga ggccaatttt aaggggaaga aaaatagacc ctatgtttca |
| 66901 | atagcaggaa ggtcaaagaa tgtggaggcc ccagaaaact gcttcccagg taagtgacag |
| 66961 | gatagaaaaa caagattttt tttttttttt ttgagacagg gttttgctct tgttgcccag |
| 67021 | gctggagtgc aatggcgtga tctcggctca ccacgacctc tgcctcctgg gttcaagtga |
| 67081 | ttctcctgcc tcagcctccc gagtagctgg gattacaggc atgcgccacc cagttaattt |
| 67141 | tgtactttta gtagagacag ggtttctcca tgttggtcag gcbggtcttg aactcccaac |
| 67201 | ctcaggtgat ccgcctgcct tggcttccca aagtgctggg attacaggtg tgagccacca |
| 67261 | tgccaggcca gaaaaacaag acttctttag gtggagtttg gggaatgagc tttctggaca |
| 67321 | agtaaacggc atgcatcaag gcacagaaac atcagagctg catttgaata aattctccta |
| 67381 | gctggtctta cctcctagct acttacttct actgttgaca tcaaccttat aaacttgcta |
| 67441 | gaggctcagt gtggggaaac aaacaaacaa agcctcatga aacgttttga tccaattaga |
| 67501 | aaatgtctat taaatacatt gtgtttcaca aatgtgaact attaatacaa tctgggtttg |
| 67561 | ttccgattta attgtctaaa attatacata attcatcttg ccaacaaatg aaattattga |
| 67621 | aaaattaaaa atatcaccac caattttttc tattaaatgt atatttttgt aatgatagct |
| 67681 | taagtttcat ctcagtttga gatttagtac actgtggcat tcactctggt tggggttcat |
| 67741 | ttggctctga gcttcaaggt tcaatttaga cttcaaagca tgtcggactt gacctgaggc |
| 67801 | acgtttttaa caatcaggtg cagtttgtaa ggttatgggt tgaggaatag cataaatggg |
| 67861 | ccatcctttt tctgattttg agaagagaat gactactaat acccaggtgt gttcttgtct |
| 67921 | ttcttgagtc aattctaaca gcattggctg tgggttataa aaccataaaa ttttacagcc |
| 67981 | aaaaacaact ttaaaggtcg tttagatgga acttcttcat tttgcagatg aggaaactga |
| 68041 | gaatcagaga caaagtgacc tcagggcatt acaacttact attactatat tgaaagagga |
| 68101 | agggaagctg gtgcaagttg agggacccac tgatgaactg cagggtatcc aatgttgccc |
| 68161 | ctgtgtttgg gctgagggag aagcagcttc ttagcttctg ctatgctgac ttatcttgtg |
| 68221 | atctctgtcc cagcccttgg ctgcctctcc tccttgtccc caggcctgca ccattccatc |
| 68281 | tgcctcctga ttattcactg acagctgcct tttccctcac cctttgttct cagtttttaa |
| 68341 | aaatccaatg gggagaagct tggattcaaa tgatagcatt ttcagctcat gaaaaattct |
| 68401 | ataacaagtg tatttgaccg tttgatctct ggcttctccc acacacttgc cctttatact |
| 68461 | ctgacagcca taagcttcat ttccttgaag cagattgcaa tttcaatgga aaggaaataa |
| 68521 | ttgtgtagct ttcaagctga gttcagttct aaaaaggact gagcaaagct caatcctggg |
| 68581 | ctattgtaga atcttagtca ctgaatcagg cagtgaaaga gctctctcca agcttcatgg |
| 68641 | ttatgatctt gaaaaacacc agagctaggt tcaaattcca gccataccat ttcctaactg |
| 68701 | tgtgattttg gcaaatcaca caaatactta aaattctttc gactcggttt cctcttctgt |
| 68761 | aaaatgggaa cactaattaa tgctcacctc aaaaggttgt tcattcattc aacacatatt |
| 68821 | cgttgggtgc ctcctgtgtg tcaaactgtt tcagtccagt gaggaagatg gacatgaatc |
| 68881 | caagagtcac caaaaaaatg taaagtggca cctgtgatga gtcacaaagg ggaggtatat |
| 68941 | gagaaagtgt gtgacagtat ctaacaggtg agggcattat ataatttatt gttgcaaaca |
| 69001 | ggacattttt gagcatgtaa gagggtgtgt taattgtcct ggaacaacag gcataaatca |
| 69061 | ggactttctg ggcaaactca ggccctaccc cactcttacc tacccttagt ataagaggga |
| 69121 | aggttgggaa aggctttcct ggggaaggga agaagaaact gagatccgcg gaataagtga |
| 69181 | ggattcctag gcaaaaagag gaagacagag tggtttaggc caagagagca aacagtggtg |
| 69241 | caagactcta aggcaggaca aagcatggag cctgcagaga acagacagaa ggtcagtgtg |
| 69301 | atgaagatgg gaggggtgcg tggctttaaa cacaggcccc ttgcttgttg gtttgccctg |
| 69361 | taggtgttaa gaaatgaggc tatatcaagg tgaattttaa gtagaggtgg cacctctact |
| 69421 | taagaccaga cttgcagctt taaacatcag tctggctgca aggcagaatg gattagcatt |
| 69481 | gagtagtagt tctggccttg gctttccaga ggaaacttct aaacaatatc aaagccttgg |
| 69541 | tgtcacccac agagatcctg atttaattag tttaggttgg ctcccagtag gttataaagt |
| 69601 | gtgtcaggat tgagattcac tgtagaagga agtgtgaata tggagaagaa taattacaag |
| 69661 | gctattgcag tagcccgggt aagaagtgat ggaataggtt catggcagtg ggtttctgaa |
| 69721 | gcagtggttc tcaaactgta gtggatttga gtatcacctg gaagtcttat ttatttatta |
| 69781 | gagatggtct cacactgttg cgtaggctag agtgcagtgg tataatcata gctcactgca |
| 69841 | accttgatca gccttcatct cctaggctca agcaatcctg ccacttcagc ttcctgagta |
| 69901 | gcbgggacta caggcatgga ccaccatggc tatttgtttg tttgttttta atagagacag |
| 69961 | ggtctcactc tgttgcccag gcbggtctca aattcctggg ctaaagcaag cctctctcct |
| 70021 | tggcctccca aagtgctggg attatagcca ctgcaccttg gcacggaaac cttgttaaaa |
| 70081 | tgcagatatc tgggtatcat tgatttagta ggtcgtggat ggggcctgag aatttgcatt |
| 70141 | tctgacaaat tacctggagg cgctagttgc tagtttagag atcacactta gcatctagtg |
| 70201 | ggtagtggtc agggatgctg gtgaatatca tacaatacac agaacagccc ttttgctgat |
| 70261 | cactgatcga gcaaaaaaac catctgatgt ggtagataac atcaacagga tttgatcagc |
| 70321 | agggtaaagg acaactccag gactcttaca gttttctgaa tttcacaact aaatgacgag |
| 70381 | tttcactatt cattgacatt caggagacag ccctacagac ctgagaagat ggtgaagtca |
| 70441 | aagctacaca tatatttgag ggttaccttt gaaacagcta agaggataca tcaagcagga |
| 70501 | acatatatgt ggaacctgga agggaagatc attacagata taaacttggg gatcattaaa |
| 70561 | gccaggaact tatcaatggc atcagtcagt tctcttttct tttactctta tcattggtgt |
| 70621 | caaagctctg gcctcttttt gttggttcca ctcaggtgcc gtgatcctcc agcctcagca |
| 70681 | ccttgcbgtg agctctcctt gggaggctgc ctctccctcc tccttcagaa tttaccccct |
| 70741 | acttctcctt tgggtcccac ctctcaggct tggtcatgtg tggcattagc aagcaggtgt |
| 70801 | gcgtgtttcc ttctgaagca caaatcaaag ccatggttat gtatctgtgg gattatttgc |
| 70861 | taaatggcca cctctcctgc tagattctaa gttgcctgca gtgactggag aacatcccta |
| 70921 | ggtatcagat gctcaatgtc ttacacttat ggaaccgtca aagacagctg ttaaatgtcc |
| 70981 | aatgggtcag ttagggagat ggtggttgtt aaaactgtga tgagttctca ggaagaggga |
| 71041 | gaagcagtta cctttgtgaa gcagttgttg gtatttactt agtaatgaga ttaaatttcc |
| 71101 | taggacagtg ggtgtgtgaa ccagcaatag cgccatctgc tggaagtaag tgatacaaca |
| 71161 | actattatct tgactaggct cagggcbgtt aaaacagtat tgctctggga gaaaagaatc |
| 71221 | atttttttta aggttaaaaa tgtgagtaaa attctttaga gtgaagatag aatttctttt |
| 71281 | gaaatggttg gaaaaattgg ataattattg aaaatagtgt tcagttttca aagaggagag |
| 71341 | aaaccttcaa gtcactgtaa ttaagagaag ttttattgaa agaacacatg gaataaggaa |
| 71401 | gttagatatc tccacccaaa accagcattt cagaaaacag aaacaggcaa aaatctggcg |
| 71461 | gagtgtggtg gctcatgcct gcaatcccag ccctttggga ggccatggtg gaaggatcgc |
| 71521 | ttgaagccag gagtttaaga atagcctggg caacatagta agatccccat tttctttttt |
| 71581 | cttttctttt cttttctttt tttcttttct tttctgagac agagtgttgc tccttttgcc |
| 71641 | caggctggag cgcaatggca tgatctcagc tcaccacaac ctcccgggtt caagaaattc |
| 71701 | tcctgccttg gcctcctgag tagcbgggat tacaggcatg caccaccaca cctggctaat |
| 71761 | ttcgtatttt tagtagagac gtggtttctc catgttggtc aggctggtct tgaacgcccg |
| 71821 | atctcaggtg atctgcccac ctcggcctcc caaagtgctg ggattacagg catgagcccc |
| 71881 | cgtgcccagc cccaagaccc ccatttctat tttaaaaaaa aaaaagaaga agaagaagga |
| 71941 | aaagaaaaga aaaatggaaa caggcgaaaa cctaggcttc tcaaagactg aagggtattt |
| 72001 | tggaactgac tggatattgc tcatctctgg ctggttgtgt tgcagcctcg ttggtggcaa |
| 72061 | gtgcagaccc tcagtcagtg acctcctttg ttgctcttcc atcaaagggc ccccttctcc |
| 72121 | tgcccaggca tggtgttact aatatggcga tggtaggggc aggggctgca tctatgacgg |
| 72181 | ggagaggtat gagagtatac tcatcattct gctgttgtct ttgttagttt gtttctaatt |
| 72241 | tctattgttg gggagaaaga taaattcttc cctaacctct tgggatcttc acctgggtct |
| 72301 | aagaattaaa tcactgtaag acaggttaac aggagaaaaa tatatgagtt ttattgattt |
| 72361 | ttacatgtac gcggagaccc tcatgagaca gcagaagacc tgaagaaatg gacaaagcag |
| 72421 | aaagctctaa taccttttag acagagaaac cataaattca tgaagagctg acaagacaaa |
| 72481 | ggggtttggg ccagtggcag taaattgtgg gaattcacta gggaggtgta aagatagagg |
| 72541 | aagataaggg atagtttaga aagtttattt gtacagctcc attgcagcat caactcccag |
| 72601 | tcaatggtga taagagttat tttcttattc tggtacaggg agggcaactt tcttaaataa |
| 72661 | atctttttgg cttacgacag gtagaagagg gcagatcaca tagccctttc cggcatctgc |
| 72721 | tgtttctcag gtgactttag ctcaaaataa tcaacatgcc acatcagcaa attttggggt |
| 72781 | gacatggcct gtttcgctat gataatggga aagaaatcag actttttaac tttgaattag |
| 72841 | aaaacacaaa ctttgactat gtcccacaat ttatgtgact gatttcatat tctatcacat |
| 72901 | ctattttata ttctctcata ttctcctcat gaattttccc ctagaatgtt gggggaattt |
| 72961 | tgaaatgact tggggggcat gctacaattc gaaaaggaga gagacatact ctccaattct |
| 73021 | ctccctgcct gcctcacaat gtgcagctct cctctgcttc ttcttcttct tttttttttt |
| 73081 | tttgagacag cgtctcactc tgtcacccag gcttgagtgc agtggcgcaa tcttggctca |
| 73141 | ctgcaagctc cgcctcccag gttcaagtga ttcttctgcc tcagccttcc gagtaggtgg |
| 73201 | tactagaggt gtgtgccacc acgcccagct aatttttgta tttttagtag agacggggtt |
| 73261 | tctttatgtt gaccaggcta gtctcgaact cctgacctca ggtgatctgc ccacctcagc |
| 73321 | ctcccaaaat gctggaatta caggtgtgag ccaacatgcc tggcctcctc tgcttctgat |
| 73381 | tcaactgctg ctaccgatag acttactgaa ggatttgaag ccaaatggta ttaattttgg |
| 73441 | tcagttactt ccctgtatct ggtgtcctca ctagacagaa cttctgtgta tggctacttc |
| 73501 | atagaccccg accagcctac atattgtttg ctctaaggtc tagtgccctc tcttatttca |
| 73561 | ctaagataca gccactgttg gtgggggagt cacataacat ggaacataat cactgtatca |
| 73621 | gttcaggtgc tcttgattca agtggattta attatgaggg aatttattcc ttacataatt |
| 73681 | actaagtcta gcattagagt ggcctgcaga tgagggttga tccagtggct cagtgatggc |
| 73741 | cagaacgctg tttacttttt ccattctgct atctatgggg ttggcttcat cctgaggttg |
| 73801 | ttttcctttc tgtcacaggt tttacatcaa tggggtaaac tagtttacat tgtggtaaca |
| 73861 | agcaactctc aacttttttt tttttttttt tttttagact gaatcttgct ctgttgccca |
| 73921 | ggctggagtg cagtggtaca gtcttggctc actgcaactt ccgcctcctg ggctcaagtg |
| 73981 | attctcctgc ttgagcttcc tgagtagctg ggattacagg catgcaccac cacgctggct |
| 74041 | aattttcgta tttttagtag agatggggtt tctttttttt ttttttttct ttttgagaca |
| 74101 | gaatcttgct ctgtcgccca ggctggagtg cagtggcgtg atctcggctc actgcaacct |
| 74161 | ctgcctcctg ggttcaagcg attcttccgc ctcagcctcc caagtagctg ggactacagg |
| 74221 | cgcatgccac cacgcccggc taatttttgt atttttagta aagacagggt ttcaccatat |
| 74281 | tgaccaggct ggtcttgaac tcctgacctc gtgatttgcc cgcctcagcc tcccaaagtg |
| 74341 | ctgggattac aggcgtgagc caccgcgccc agccaactct caactctagt agcttaagac |
| 74401 | aacaaagatt tatttcttgc tcttactgtc tgcccatctg tatcaccttc tctgcaggac |
| 74461 | ccagtttaat ggagcacaca cagaaaaagg tgctggtcac tagggccaag ggaacaatgg |
| 74521 | ctctggaagg tttttcttga acaattcagt gttccagtct tggagcaata catcttattt |
| 74581 | ctactgttaa atccatggcc agaactaatc atgttgtccc actcaccaca aagccatatg |
| 74641 | cagccccacc atgtgctcag gaaagaggaa tgggaagcat ttgatgaaca gaaataatga |
| 74701 | cttcaacaat tgggactatc agttcacagt ttcaattgta aaaaaaaaaa aaaaaaaaaa |
| 74761 | agacagaaat agaaaaagaa caattgaaag tacttgcttt ctcatccacg aaaaaagttg |
| 74821 | gtcaaagcct cttagtgtaa gtctatagca gatgatgcct gataaggaat tgtacatacc |
| 74881 | gttcttcgtg aaaaccaaga gtaccatgta tgtaacatag ttgctttggg cttcttatag |
| 74941 | cagttaagag tgtatcagca ggcctccatg gtatttgaat gttaaaagct ttcattcccc |
| 75001 | ctggaagaaa acacatagct aactaaagaa agaaaaaaga agggaaaaag aacaaagaaa |
| 75061 | aagaaccact agagtctctg aaaagagctt atgagatacc tgactgtctg cgccttctaa |
| 75121 | gtatgtacca tgaaacctta gattcctacc tcattttgtg atggtgtaga aggcacattg |
| 75181 | gccaccagca acccttgtaa atatttgcgg atttgaattt cacaaattga aacccagact |
| 75241 | gctgagggct ttgctgctaa cgtcaaagag ctggggcaac taccacaaaa ccacatgtgg |
| 75301 | gagacttttt agatgaggag ataggaaatt cctaataaga aaagtttttt tgtaaaatac |
| 75361 | tttctaatgc tggctcttta gagaagcaca ttttacatgt gcaatggtgc aaagtcctag |
| 75421 | cactaagatt tcttcactca cagaaagaaa gaatgatgca ggtgctgttc acatgcggcc |
| 75481 | ggaaccatgc ccttgccaag caggtcagcg gggcacaggg actgcgctcc tcatgatagt |
| 75541 | gcagattggc tcggaagtac tcaattctgt ggagtcacag cgccctctga cagatttcag |
| 75601 | tttctggaaa aggaagggcc cccttcaggg tacctgctgt tcaccttcca ttagattaag |
| 75661 | gttgaagagc gagtggccat ttgtaggtac tggtatgaga tgttcctagc tacccgcatg |
| 75721 | tcagccccgg gaaacggcaa catttcccac ccttcttacc ctgggtcctg agtaggaata |
| 75781 | aaagttagtg attcttaggg gcatattgag agctggcaat gatacctctc acaaatccat |
| 75841 | cccccagaga taaggggtcc cccatgcctt tgatgtattt gggagcbgtg ctgtatgtgt |
| 75901 | tctctttggc tccccagtgg agaataataa caataacaac cgcgtactaa tacaacccag |
| 75961 | atgtcagcac agaccctctg ttctctgtct tccctgagat atctgtatct tctcagggat |
| 76021 | tactgtcggt tcaggcctgg ctcctgcttt cagcttgtgg gaaaactcac tccatcttca |
| 76081 | ttccataacc tcacatctcc tatgccagtt cccttctctg tgctccatgc tgaaccaaat |
| 76141 | gccacactca tcccctttgc tttgaattat atgaaaaaaa gaaaaaaccc tcatggggac |
| 76201 | tttatgtgta acagtcttca ctgcacattt gataacacag atgggggttt atatcctagc |
| 76261 | agagcagata ttgtcaatgg aaaatggaaa caaaacaaaa cacaaaagac aatcttcagc |
| 76321 | catcactatg tagcttggct atcatctttt gctttctcct ttacttctgg agtcccccca |
| 76381 | cataccctca gaaatggaga tagactctac atcaatctct gctctgggaa atctactcac |
| 76441 | atatccaaag gcaaactaac aagctcttgc tccgacaacc catcgacttg gggtctcatc |
| 76501 | cctccctgcc taccagtctc tgtgactgaa aggggcccat acagagactt attttcccag |
| 76561 | gaacgtgaat gatatccttc tcctgttcct tgaccttggg aggcagattt gattatggtc |
| 76621 | agctttgatt caaagtaagt tttttttttt ttctccctta ggagaatgtt gtgaggtgct |
| 76681 | gattgttctc tgcttcataa tcgtatcact tattactgat aatctttttc tgcccccaga |
| 76741 | ggttgctgaa gaatgtagca tatacagtgg acacagagcc ctgataatac aattactaaa |
| 76801 | ttctccaagg ttgagggcct ggtcttaggt catgagatta ctgatccttg ccttctgcct |
| 76861 | gctaggaagg cccaactaat ttcacagtct ttatcagtga accatagtaa agggggacca |
| 76921 | gaagagaaaa aggttatgtc ttttgggaaa atgccaaaat catattgtga aaatccttcc |
| 76981 | tactgttctt ctcatgggag ggggaggact ttggacttgt ttctctcctt ttcctgtaca |
| 77041 | tttggggatg tcagaaccgt ctgtttcctc atctacacgt ggttagatgc cgacaacaat |
| 77101 | ctcttctctt aaaattgctt actattagac cagcagagtg tttggtatta attgagcaag |
| 77161 | gaggaatttg taggaaaggc ttggcttaac taggagaggg aatgccaggg agaaatctgt |
| 77221 | atttcccact attttttttt ctttttttga ggcacagttt cactctgtcg cccaggctgg |
| 77281 | agtacagtgg tgcaatctca gctcactgca acctccgcct cctgggttca agcgattctc |
| 77341 | atgcctcagt ctcccgagta gcbgggatta caggcgtgtg ccagcacacc cagctaattt |
| 77401 | ttgtattatt agtatagatg gggttttatc atgttggcca ggccggtctc aaactcctgg |
| 77461 | cctcaggtga tctgcccacc ttggcttccc aaagtgctgg gattacaggc atgaaccacc |
| 77521 | acacctggcc atttcccact attttgttga taaagacatt tggatttccc tgcattaggc |
| 77581 | agatgtggaa gaagatggaa gcgatgaaaa ccttcttgcc aaaatctccc aaaagacctg |
| 77641 | gccagtgggt gagcagatct cagttacaaa aagcctatag gttgaatttt ctaagaaaag |
| 77701 | gcctggggag gaggtaccag gagtccaaat tgaagttcga ttttcattat cacagatttt |
| 77761 | catatctctc tatctccctt tttttgtttt tggtgagaca gagttcaaga ccagcctggt |
| 77821 | caacatggtg aaaccccgtc tttactaaaa atacaaaaaa ttagccagcc gtagaggcgg |
| 77881 | gtgcctgtca tcccagctac tcgggaggct gaggcaggaa agtcacttga acccaggaga |
| 77941 | tggaggttgc agtgagccga gatcatgcca ttgcactcca gcctgggcga caagagccaa |
| 78001 | actccatttc aaaaaaaaaa agaaagaaaa agaaaaaaga aaaaaagaaa atgcactttt |
| 78061 | tgcbgtcagt aaaagttaag caaaagtctt gctgagagac tgatatttaa ataactatta |
| 78121 | taatgcaatg taaaattact gcaagagata tgaacacagt gctcaggaag cttagaggaa |
| 78181 | ggagaaacca agtccttagt gatggaaggt gggctgtcaa ggaaggcttt gtggaggttg |
| 78241 | tgtaattgta ggatgagggc catgtagtag gtcgacgggg ccctcgtgtg ccaggtggaa |
| 78301 | ggaagccatt taagtctttt aagctgagtg gtggcatgat tacaagtgca ctttagaaaa |
| 78361 | atctctatgg cagcaatgtg gaggatgaag taacaaaaat atacactttg gttcattttg |
| 78421 | ttctaaatgg caactttacc tttaactgtg tacagagctt actgactata ctcaagcaac |
| 78481 | aaaatgcaca cttggaaatc ctgtgtcacg tgcctatact agctttgtcg ctgattcagc |
| 78541 | atatacacat cctccagtgt gacaggattg tgtatggaaa caggtaagac tggtgaatac |
| 78601 | taggctgtgt tcaagtgtag agtctcatga ttatcaccaa tactgbgggc tctccctctt |
| 78661 | ttagatgagt ttttctacca tatctttggc tttacaaaat atattaacac agtttcataa |
| 78721 | tagatctctt ggaatatggt cgtttggtgt taagcattct atctggcaaa atgaaaaata |
| 78781 | agatggtaag agttatccta caaaaatcct aatgttctca agctactgct gaaattcttt |
| 78841 | ttctagtaat ttacaaaaaa gtccccattt tcttgtagaa ataagccaat taaaaaaaga |
| 78901 | aaaaaattaa aataaaaaat taaagttctt aatgataaat tatattaaaa tttatttcct |
| 78961 | gttttaccct atatcctatg agcgtggttc tcaaacttcg ttcagtgcat accagaatca |
| 79021 | tcatgtgcgc ttgttaaaca gaggtctggg ttccaccccc agagcttctg attcacttca |
| 79081 | tggaatctga gaactggcat ttctgacaag ttcccagggg atgctgaggc tgctggtcca |
| 79141 | gggaccacac actgagaaat attgttctga aataataaca gctaattttt gttaggcagt |
| 79201 | ttctgtgttc caaatacagg ttgaatatcc cttatccaaa atgcttgggg caagctgggt |
| 79261 | gcagaggctt attcctgtaa tgtcagcact ctgggaggct gaggcaggag gattgcttga |
| 79321 | gaccaggaga ttaggaccaa cctaggcaac atagcaagac actgtttcta cacacacaca |
| 79381 | cacacacaca cacagacaca cacacacaca cacacacaca aattgccagg catggtggca |
| 79441 | tatccctata gtcccagcta cttgggaggc tgagggagga ggatcacttg aaacaaggag |
| 79501 | tttgagacta tcctgggcca catagtgaga ccctgtctct acaaaaaata tttgaaaaat |
| 79561 | tggccaggtg cggtggctca tgcctatagt cccagcactt tgggaggctg aggcaggtgg |
| 79621 | atcatttgaa gtcaggagtt caagaccagc ctggccaaca tggtgaaacc ccgtctctac |
| 79681 | taaaaacaca aaaattagct gggcagtagt ggtgggcgct tgtaatccca gctactcagg |
| 79741 | aggctgaggc cagagaatcg cttgaaccca agaggtggag gttgtagtga gctgagatcg |
| 79801 | tgctcctgca ctccagtctg ggtgacagag tgagaccctg tctcgaacaa aacaaaacaa |
| 79861 | aacaaaacca aaaaaaccaa aacaaaaaac atattagctt ggcatggtgg tgcattcctg |
| 79921 | tagtttcata tgtgatctca ctactgcact ccaatgtggg tcacagagca ggactcaaat |
| 80041 | gaaaacaaaa ccaaaaccaa aattcttggg accagaagta tttcagattt cagattattt |
| 80101 | tggattttga aatattggca tgtatctaat gagacatctc agggatggga cccaagccta |
| 80161 | aacacaaaat tcatttattt ttcatataca ccttgtatac atagcctgaa ggtaatttta |
| 80221 | tataatattt taaataattt tgtgcacctg tcacgtgagg tcaggtgtgg aattttctac |
| 80281 | tgtggtgtca agtcaatgct caaaaaattg cagattttgg agaatttcag atttcagatg |
| 80341 | tttggattaa atgtgcttag cctggacctt acttgttgta gctcgtttaa tcctcatggc |
| 80401 | aatgctagga cacaggtggt tttagtataa tattattaca aatgaagagc ctgagacttg |
| 80461 | agaaagtaag acaacacact ccaggtcact cagccaatta gtggtgagca agaattgaac |
| 80521 | ccagatttga tatgcgaatg acttgctgtg attctcctat tggtgatttt tttttttttt |
| 80581 | tttttttttt ttgagacagg gtcttgctct gtcacccagg ctggagtgca gtggcatgat |
| 80641 | ctcagctcac tgcagcctct gcctcccagg ttcaagcgat tctcctgcct cagcctcctg |
| 80701 | agcaactggg actacaggca tgtgctacca tgcttggcta atttttgtat ttttagtaga |
| 80761 | gatggggttt caccatgttg gccatgtgga tctcgagctc ctgacctcaa gtgatccatc |
| 80821 | cgcctcggcc tcccaaaatg ttgggattat aggcgtgagc cactgcaccc ggccccttat |
| 80881 | tggtgtattt ttatttccca agtatgacac ctgttcagag actttggaac cctgagaaca |
| 80941 | ttttgttggt gtgttttgtg actaatattg cctttttctg agatgaatgc tgttctttga |
| 81001 | aaaaacattt gtatttttag atagaaatgt acttcatccc cagtgaatat ttctgacttt |
| 81061 | agagaaattg ccaaacctgc tgactcataa caaaaataaa gagcatctac tattcagttt |
| 81121 | atggctttta gttttgggag gtggtaggag gaaggataga ttaccaccca ctatagatca |
| 81181 | ttttattttc attttatttt aatgccattt accagtattg tgtatatgga ggtggtcttg |
| 81241 | agagctttgt ggagattatt ttttcaatag tacagatggc tctgataaat atttaaggca |
| 81301 | tcttgggaaa caagaatacc atcctgactc aggagaaatt ggtcacgtaa atcactttgt |
| 81361 | ttcaagtgct ttggcctctg agcatctcaa atggacacat acttcattgg cttattaact |
| 81421 | cattgccgga gtcactgaag gcccaaacta caacttcatg cctgaccaca caccaaggct |
| 81481 | tttttattgc ttagttttct ggctctttgc ctattctgct ctccctaccc ataaccatgt |
| 81541 | gagtatcatg tccagcaaac taacttactc cttctctctc cccactacac acacacacac |
| 81601 | acacacacac acacacacac accacactcc acacgcacac atacacagag catttttcat |
| 81661 | gggtatgttg tcagacagat ttctctgttc tgaaggctca tctccttaga gaaccttacc |
| 81721 | gtgtcaatgt tgcatttagt ttagtttagt gttgttgttg ttgttgttgt tgttgctgtt |
| 81781 | tttaaatctt gctctgttgc ccacagtggc gcgatttcag ctcactgcaa cctctgcctt |
| 81841 | ttgggttcaa gcaattctcc tgccgcagcc tgctgagtag ctgggattac aggcccccgc |
| 81901 | caccatgtcc agctagtttt tgtattttta gtagagacgg ggtttcacca tgttggctgg |
| 81961 | tcaagtcttg acttcctgac ctcgtgatcc acctggcttg gcttcccaaa gtgttgggat |
| 82021 | tacaggtgtg agccactgcg accggcbgtt tttttttttt tttttttttt ttttttcttt |
| 82081 | tttgagacgg aatttcgctc ttgttgccca ggctggagtg caatggtaca atcttggctc |
| 82141 | attgcaactt ctgcctcctg ggttcaagca attctcctgc ttcagcctcc caggtagctg |
| 82201 | agattgcagg tgcccaccac catgcctggc taatttttgt attttagtag agatgggatt |
| 82261 | tcaccatgtt ggtgttgtat ttagttttaa cactgttatc ttctcagatt gtaatgagag |
| 82321 | aacaggctat catcacttgg tgataaaatt tcctgtaatt ataatctcat tctcaaagat |
| 82381 | aaaacattga tacctaatgt gcttttaact ttaatgccaa tttatcacaa tggaagacat |
| 82441 | ttcaaataaa tactgttttt ttgttcataa ggtcttaaaa atcttgaata taactgcaaa |
| 82501 | aaagcattac atgttaaaag aaatcacaaa attagttaag aaaggagcca aactgaaaga |
| 82561 | agattatatt tctatgccct tgaatcttta caaaatttag taaaaactat taatatagaa |
| 82621 | ttatttacaa gagacttctc tatttaccag cccaatatct tcacatttct acaatctaag |
| 82681 | gggaggtgat tgtggccaga aaagtttctg ggctctggta ttgcagtttt cctgcactta |
| 82741 | ccaccatccg gcatgttata tattttactt atttttcttg cttattgctg tctctttcca |
| 82801 | caagttccat gagcagagac agtttatctg tgttttttac ggctattctc aatgtctgta |
| 82861 | acagtgtcta acgtactata gtcattaaag aaatgctggt tgggtgaatt cagaaataaa |
| 82921 | ttagtaaata ggtgaagcat gttggtttaa tattcatcat cagaacttaa aaacagttta |
| 82981 | aagaacctca actgagtttt gtacaataag tagtgacaag gtgggacaga ggtaatttct |
| 83041 | ttgtatctct gaggctttcc atttcatgag gaacatagaa taatatgtgt ttccaaatgc |
| 83101 | agtgttcaat atctgaagag tatatatcat cagtatagaa aatagacaac acatgttaaa |
| 83161 | gggaatgtaa aaattctaga tgatttttaa ggtatgtatg gtttaatcaa ttgtttatat |
| 83221 | gctattctct tctcttaaaa tgaaataatg cgaatgaaaa aataaacctt acaggtagtt |
| 83281 | tctgggaaat tcagacaaca aaattgtcta acatacgcca aaaacaatat agcatttaat |
| 83341 | gatgacatgg attccgaaag acaaatcact gctcatcata ttacaaggaa tccaaacaac |
| 83401 | tgaggggtta tcatttatct atagttgcat aacaaattac cctaaaattt agcagcttaa |
| 83461 | aacaacaatt tattatttca taaatttctg gggatgagga atccagaagc agattcactg |
| 83521 | ggtggttctg gctcaaagta tcttatttgt ttgctagggc tgcbgtcaca gaacacaaca |
| 83581 | gcacaggtgc cttaaataac agaaacatgc attctggagg ccagaagtcc aagaccaagg |
| 83641 | tttcctctga ggtctccgtc cttggattgc agatgcttgt cctcttgctc cctcttcaca |
| 83701 | tggttgtccc tctgtgcaca tgcacttctg tgtctctttt tgagtgaaaa tttataagca |
| 83761 | ttccagtcag attaattttt tttttttttt tttgagatgg agtctcactc tgacacccag |
| 83821 | gctggagtgc agtggtgcaa tcttggctca ctgcagcctc cacctccctg gctcaagttc |
| 83881 | ttctcctgcc tcagcctccc aagtagctgg gattacaggc atacaccacc acgcctggct |
| 83941 | aatttttgta tttttagtag agacgtggtt ttgtcatgtt gcccaggctg gtctcgaact |
| 84001 | cctggcctca agttatccac ctgccttgtc ctcccaaaat gttgggatta caggtgtaag |
| 84061 | ccattgcacc ctgccccagt cagataaaat tttaatctca ctttaactta atcacttctt |
| 84121 | tgaaggccct gtctccaaat atagtcacat gaattagggc caagctgtat ggcttcattt |
| 84181 | aaccttaatt acttctttag aggctctatc tgcaaatgcc tccacattct gaggtggtgg |
| 84241 | gggggttagg atgtcaacat atgaatttga gggggcacaa tttagcccat agacacctgt |
| 84301 | catgaggttg tagtcagctg tcagttgggg ctgcaatctc atctgatgga ttaactgggg |
| 84361 | aaagatgggc ttcccagctc actggtgtag ttgttggcag gcctcagttt cttctcgctg |
| 84421 | gagacttcag ttcttgccat cggcttctcc acaggttgtc cagattacct aatgacatag |
| 84481 | cagctggctt ccccaaaagc atggaatggg ggttggggat gggataagag agagagaatc |
| 84541 | caagacagaa tgcacagtct tcttataact taggtttaga aataatatat cagtacctct |
| 84601 | gccacattct gttggtcaca cacactaacc ctggtaaaac atgggagtga accatacagg |
| 84661 | gggaactggg gtcattggtg gtcatctttg aggctggcta tggtaattag gtaaaaataa |
| 84721 | aaattcagtc tgcagaaacg tatgtactta tgttacttaa aatattttaa ccaattagtg |
| 84781 | aaaaatgact ttgaagtatt tttagctgct ataataattg agtaggaatt attttttctt |
| 84841 | ggattgagtt atatttacta taaaaatact tacttttttt gtcaaacaca tttataagtg |
| 84901 | ctgcaaatat tagtgaccaa tggtctattg ctctaaataa aaagcaccag gagttagtat |
| 84961 | agtagttgac tccagcctct ggtgctagac tgcctgggtt tgaatattta cttttgcact |
| 85021 | tacaacttgt aacaacttga acaatttatt caatctctct gttccctagt tttctcatat |
| 85081 | gtcaaatggg agaaacaaat aacatctgct tcatatggct gttatgaagg ctaagtaggt |
| 85141 | ttatatatat gtatgtatgt atgtatgtat gtgtgtgtgt gcatatatat atatatatat |
| 85201 | atagagagag agagagagag agagagaaag agaacttaga atagggcctg gcacagagaa |
| 85261 | tatgataaaa gaatattatt tgtttcctct ttttatcatc cagaacatgg tgtaatgccc |
| 85321 | aatacattgt agatactcaa taaatgtttt tgtatgatcc ctaaacattg tctgtaagga |
| 85381 | atttacaatg gagtttacag taaaaggtgg tacatggtag gtaccatgga agcattacaa |
| 85441 | acagatgacc atgtgaatct gaaagaagca cagattgatt tcttcgtcat aaaacatggt |
| 85501 | tgtatttgag ctaaagctta gaactaatga agattattgc caggcgtggt ggctcacgcc |
| 85561 | tgtaatccca gcactttggg aggccgaggt gggaggatca caaggtcaga agatcgagac |
| 85621 | catcctggct aacatggtga aaccccgtct ctactaaaaa tacaaaaaat tagctgggtg |
| 85681 | tggtggcggg cgcctgtagt cccagctact caggaggctg aggcagaatg gtgtgaaccc |
| 85741 | aggaggtgga gcttgcagtg agccgagatc gcgccactgc acttcagcct gggcgagaat |
| 85801 | gcaagagtct gcctcaaaaa aaaaaaaaaa aaaagagtat caatgtatca aaatcattat |
| 85861 | tttaggctgg gcatggtggc tcacatgtgt aatcccagca ctttgggagg ctgaggtgag |
| 85921 | agatcacttg agcccaggag ttcaaggcca gcctgggcaa aatggtgaga ccctgtctct |
| 85981 | acaaagaaaa aaaaaagatt attattcccc aaataggaaa taacataagc aaacgtcccc |
| 86041 | tatttccccc acgatggagc tggagcatgg tgtatgagaa ggagagtcag agggagcaag |
| 86101 | aatgaagggg tcaggttggt accaggtaat gaaatcattt taacataaga ctacatgatt |
| 86161 | tggacttcat tgtgtggtca cttcggagca ttttagcaac aaaataatgt cttaggaaga |
| 86221 | ttaatctggt ccaattgtaa agtaaattga gattcaggct tcaggcttgg cttgatccag |
| 86281 | gattcaaata aaatcaccaa gaatctgtat ttcagatctg ctttttttgt gtggctctgc |
| 86341 | ctgtggctcc ataagaagca cagaaaaaag agaaatggtc tgttaaacag aaaaagtgaa |
| 86401 | aaagcttcac tcccagaaaa tatcttcttg catctcatcg gcctgagttc actcatgttc |
| 86461 | gtattcttca acaaattatc atgtccagga tatgaaacgg ctgcttggat ccaggcacac |
| 86521 | tgataacgct gataccacac ctagaaaaag aaatgaattt cattcaaatt ccattctact |
| 86581 | attttaaggt ttcatgtcta gatggctgga gattaagtgt tgcagtccag gcgtggtggc |
| 86641 | ccacgcctgt aatcccagca ctttgggagg ccgaggtgga cagctcactt gaggacagga |
| 86701 | gtttgagacc agcctggcca acatgatgaa accccatctc tactaaaaat acaaaaaata |
| 86761 | gccaggcttg gtggtgcgtg cctgtaattc cagctactcg ggaggctgag gcaggagaat |
| 86821 | cacttgaatc caggaagtgg aggttgcggt gagccaagat ggcactactg cagtccagtc |
| 86881 | tgggtgacag agacttcatc tcaaaaaaaa aaaaaaaaag taagttttgc aattaggaga |
| 86941 | catgcctata atgtttattt agtggtgata cgtatggttt agtcctttaa ttatagtgtg |
| 87001 | gtttaaatgc aaatttaaag taactcaaat acagctattt tttctcacat catagggaga |
| 87061 | ctataaagtc cctcagtaat cattcttcaa atagatattt ttcttctttc atctactgcc |
| 87121 | ttcacgactg cagtcacttt ctccaacagt cttttctcca aataaaaaca aaaacaccac |
| 87181 | taccaccact accaaaaatc tcaagtacct gctatctctg atctttctca tctgctaata |
| 87241 | ttctcttcct cccttactcc cctcctcttt tcttcctttc ctccttcccc atctctccat |
| 87301 | ctcacacctc cctctcttcc tacctccctt tctttcaacc ttttcccctt ctattcttcc |
| 87361 | ttccctcgct tcctccttcc cttccttcct tccttccttc cttccttcct tccttccttc |
| 87421 | cttccttcct ttttgctatt aaaaattcct caaactggct gggtgtggtg gctcatgcct |
| 87481 | gtaatcctag cactttggga tgccgaggtg ggtggatcac ttgaggtcag gagtttgata |
| 87541 | ccagcctggc caacatggtg aaaacccgtc tctactaaaa atacaaaaat tagtagtgta |
| 87601 | tggtggcacc gcacctgtag tcccagctac tcagaagggt gaggtgggcg aatcacttga |
| 87661 | acccaggagg cagaggttgt agtgagccga gatcgttcca ctgcattccg gcctgggtga |
| 87721 | cagagtgagt gagactgtct caaaataaca acaacaacaa aaacaaactg gcaatgcagg |
| 87781 | acttccttga aagacagagg aaaggaaaag attaagagaa gttataccaa aactcttttt |
| 87841 | aataggtagg cagggaaaaa attgggaacc tttatacatc aataatttta ttctcataat |
| 87901 | acttggaaat catgactagc tgttgctctt cttcaacatc taactattat catttccaca |
| 87961 | tctgattcaa gcacttcctt tttacatgtc tttttttttt ctttgcctga aatattattt |
| 88021 | ccttccatca aattaacctt tctgcccacc tggcaaccct ctcttcttca agccttactc |
| 88081 | atcaagaagc cttccctagc aacagggaag gctctgctac caatcccacc tttatgctct |
| 88141 | ctggactacc actttgtctt gtacatttct attaaattgt aacttttcat ttttgttaca |
| 88201 | catctattga ctctactgct cagtgaacta tttgagacca ggacaccatt ttgcatacct |
| 88261 | agcacaatac ctaccacata gtagacattt cataaatcct tattgattga ataaatcaat |
| 88321 | gatgaataac atttttataa atttcatttt gtatttgagt tgaagctcca agtattacac |
| 88381 | cctgttagag attttaaact tgatttttaa aatattggac tagagcttaa aagaagatta |
| 88441 | tattagtcag gggtctcaag agaaacagaa tcaataaagt atacatttat tatataattc |
| 88501 | attataagaa attggctcac atgattgtgg aggctgacaa gtctcaatac ctacagtcgg |
| 88561 | caagctagag acccaggaga acctatggtg tagtccaggc tcattccaaa ggcctaagaa |
| 88621 | ccaagcgagc caatgatgta gttctagtgt gaaaacctgc aggctctaga cacaggaaga |
| 88681 | agccaatgtt tcagttaaag tccaagggca ggaaacacca aggtcccagc tcaaacatag |
| 88741 | tgagacagaa ggaattctct cttactcagc cttttgttct attcaggctt tcaacagatt |
| 88801 | ggatgaggcc cacccacaat ctgctttacc cagtctactg atttagatgt taatctcatc |
| 88861 | cagaaacacc ctcacagaca cacctagaat aatgtttgac caaatgtctg ggcaccctgt |
| 88921 | tacccagtca agtggatgca taaaattaaa caccataaag actaaagcct gtagatgaat |
| 88981 | atctggtaac acctcacaga ggagagacct aaagctgtga taacgtctct gaagccaaga |
| 89041 | gagaaaggct agaaaaagca agatctggga tatttcctta gaatccttgc ctgaaggcca |
| 89101 | agaagatgag gaagttaatt ttctcatcca gagtggtggt atcgagctag aaaattgtga |
| 89161 | agatctttct agttctaaaa catctgttac ctccttacct tggggtttca agactgggta |
| 89221 | aatggaaata ctaagataat tggatgatgt aagaagattt tggatgggaa caatatcttg |
| 89281 | ttatgcacca gttgtatgga aactacttat ttataagatt taaaattcta cgtatattcc |
| 89341 | ttcaggaaat gttgtcggct ttctacaaga tgcagcttta attgtattat tgggaaccat |
| 89401 | cattgattat taggttcttt ggagaaattt ctttccttca attagaagca cccatgaagg |
| 89461 | atatgaagaa gagttggtca aagaactata agaagatcat agaatgtact gcactataga |
| 89521 | agccaaagga agaggtaaat caagaggcaa gaaatgatgg aaaggtattt tgctatagag |
| 89581 | aggtaaaggt tgataagact tgggaaaaag gccatcggat tttacgatta ggatattaag |
| 89641 | gacaagtttc agccaagtca tgagaaactg agcatcagtt aggtccctgt tgtttgatat |
| 89701 | tatgacaatg tggtggattt agtaattaga gtctttcaaa attgtctgtt agtgctttgc |
| 89761 | agcttggggg catgagcaga gaaggtaggg ggcaggagga atggggagca tgtctgtggc |
| 89821 | tgagagttga aggcttgaat ggtaagagta ggcttgagga ttgtggcttg ttagaaaaga |
| 89881 | cagcattaaa atatctgaac atgggacttt gtttttagaa aggatacaaa tgaagttaaa |
| 89941 | agccagaaga aggtcttggg tgggattttc aagctgcatt ccttggagtc ttgaatcgtg |
| 90001 | gattagagaa aagaccaccc tgtgttgatc tcagtagctt ctcctgttaa acatcacagc |
| 90061 | acatgtgctt tgaccagttt tgtatattaa ggctctatgt ggctatcttc tgtcccgctc |
| 90121 | tgtcccccac ggccattctc tttccttcac catgctgctc tatgacccgg gaggctgacc |
| 90181 | tatatggacc atatcagtgg gctgctttgc cctcctgctt ctgtttggat tttgcccttg |
| 90241 | gggagcactt gcatgagctt gcagggaggg agaatgagag gcaaggtata atctgctgtg |
| 90301 | acctacaggg tcatcaggat cctttcccct tcaggcctag agttggtaat aatgcccagc |
| 90361 | tttcttaggc ccttgaaatt tctctatacc ctgctatatc tgttcaattg cctacataac |
| 90421 | ttaaccagtg tatgcttact actcactgca gccttgatct ccctggctta ggtgatcctt |
| 90481 | ccacctcagc ctcttgagta gcbgggacta caggtgtgca ccaccatgcc tggttaattt |
| 90541 | tttgtatttt ttttgtagag acagggtctc accattttgc ccaggctggt cttgaactcc |
| 90601 | tgggctcaag caatcctccc accttggccc cccaaaatgc tgagattaca gacaagagcc |
| 90661 | accactcccg gcccaaacat tttttttttt tgtaaaaagc caggcagtaa atatttttga |
| 90721 | tctgtgagtt atacaatctg tgttaccacc acttaactct gctatctcag tgagaaaaca |
| 90781 | gccatagatg ttaagtaagt gaatgaatgt agcbgtgttt caataaaact ttatttacaa |
| 90841 | aaacaggcag tgagctttgc ttttggccca taggctataa ttgaaagacc catgatctag |
| 90901 | gacaagaagt tttccatcta ctctatgaga gtggttctca atctgggata atttagctcc |
| 90961 | ctaagggaca tttgttaatt taagacattt ttggttgtca caactggagg agtagtgcta |
| 91021 | ctggcatcta gtggataaaa tccagggata ctgctaaaca tactgcaatg cacaagacag |
| 91081 | cccccacaac aaaaattacc ctgcccaaaa tatcaatagt gctgaggttg agaaatgctg |
| 91141 | gtcttgaaga tgttaaaaag attgtcagtt tgttcatcat atcgctgtgg cttggcgaaa |
| 91201 | agaatcacag agttaatttt ataactatga aggattgtta ggagctgagt ttttttctag |
| 91261 | ttcttatttt ttaattattt tatgatactt ttcataaaaa gtggtgtaag ttgtgtttat |
| 91321 | tcagaaatgt gatttttttt tttttttttt gatggagttt cgctcttgtt tccgaggctg |
| 91381 | gagtgcaatg gcgggatctt ggctcactgc aacctccgcc tcccgggttc aagcgattct |
| 91441 | cctgcctcag cctcctgagt agcbgggact actggccacc atgcctggct aatttctgta |
| 91501 | tttttagtag agatggggtt tcaccatgtt ggccaggctg gtcttcaact cccgaactca |
| 91561 | gatgatccac ctgcctcggc cttccaaagt gcbgggatta caggtgtgag tcactgctcc |
| 91621 | cagcccagaa attgatggtt tctttagcaa ttttgtcacc catcaagtcg ttagtcatat |
| 91681 | taatcctcca taacttagtc tttttgcccc ctgattatct tttgggtggg cagtgaaagt |
| 91741 | attcatacct cttttagcaa aacctatctc ttcttggatt tcctttgctt gctgattaag |
| 91801 | aggaaggact atctgtgaaa gactcagatg tggctcaatg gaatgaatgt gagaatggta |
| 91861 | tgagtgtggg tggtgggtgc tgtgggggtc ggagatgagt ggctgaacca tccaaaaata |
| 91921 | agaatactga caagcgtgtt ttcttgtgtg tgtgtgcgtg ttgtgggagg agtgtaactg |
| 91981 | atgtggctag acaggattaa cgaaagtctc aggagttgag agagactggg gacattttac |
| 92041 | caatggcgag aaggaggaag gatgacagct gggaaaatta gtgattggaa aaggtaggct |
| 92101 | gggcagtgtg gccatgagct agtccttttc attatattct tctggggctc tatgtttaaa |
| 92161 | actagatact ttaacatttc agaatgagaa agagaggaaa ggatggtaac tgatatgggc |
| 92221 | tattatactg tgaattttaa aggagcctat atattatcct aaaactattg aacctgagca |
| 92281 | gaactgcaac gttggtattc ttgaactgaa ctgatatttc ctttgactaa tgtccttgtt |
| 92341 | caatgaccag agaccatgac actccccacc ttcaccccca cacattgttt gtttgtgtcc |
| 92401 | agtttgaaga acaatgacac atggaggtca gtgtttggat gatgtcataa ttttgatcag |
| 92461 | ctggaaagcc tggagtggcc ttgaagagat gcttaacttt tggaaatcac ttctttattg |
| 92521 | caaccaaagc tattaagttt aaaggtccat aaagatgaga taaacagtca taaaggcctt |
| 92581 | ttgaaattca tacttagtgt gttaaatgga tttaattgct gttagttaaa tgtggtgatg |
| 92641 | ttaaagcaat gtctatacac tggcaaaaat cagatcatca actgcttaat gagggacacg |
| 92701 | tcttgagaaa aacagtggaa gaaatgctgc ttatatgtaa tttgtgatta atttttgcaa |
| 92761 | ggttttcaag ggaataactt ggccttaaaa tattacagtg aaaaacagct ttatacttgg |
| 92821 | gtccaatctt cttaaaatta gagttcattt ttgctattag acacctttta ttacaggata |
| 92881 | actttttctt cccccttgga gacagagtct tgctctgttg cccaggccag aatgcagtgg |
| 92941 | cgtgatctcg gttcactgta accaccgcct cccaggttca agtgattctt gtgtctcagc |
| 93001 | ctcctgagta gcbgggatta cagtgcctgc caccatacct ggctaatttt ttgtatttta |
| 93061 | gtagagacag ggtttcatca tgttgctcag gctggtctga aactcctgag ctcaggcaat |
| 93121 | ccacccgcct tggcttccca atgtgctagg attacagatg tgagccactg agcccagcca |
| 93181 | gttataggat agcttctaaa cttacaatcc ttgaaatatc attttaggat aattctgatc |
| 93241 | ctgcatacca gtgaaataag tataattgaa atattaaacc aaatttggct tcaacattaa |
| 93301 | accaaaatta aaccaaatta agccagtatg ttaaatacat agtttaatct taattctagt |
| 93361 | taatattttt catttcctat gtgtcaggca atatactaaa caacttacat gtattactat |
| 93421 | tttaattctc ctggtaaact ggaggggtgt gcaattattg tcatctccat tttatagatg |
| 93481 | aagaaaactg agtcataaag aggttatgta cttggctgaa agtcacacag tcagtgatac |
| 93541 | tgttggaatt tgcacccaga caatttaatt ttagagccta ttttcaacca ctctctgaga |
| 93601 | ctgctttcct tttctgtact tttgatgata aatgtgaaaa gataattttg agggcattat |
| 93661 | ggtgtattgg atcttaaaaa cccaggtaat aagtcccatc tatagtacaa ataagctgcg |
| 93721 | tgatcttagg gaaatcacat aactgcttct gtaaaatgag agattacatt aagttatttc |
| 93781 | caaaatgtct tttcgacttc aatgtggtga ttccatagta atgtataggt ttttaaaaaa |
| 93841 | ccatatcctt caatcaaatg gtcatccttt agcattaagt tgtttggaat agatactccc |
| 93901 | caaattcaca gaataatttt ccagcctctc tttttgcttt gacacctttc ttattttttt |
| 93961 | tttttttctt cgagataggg tctcactcag tcgctcgggc tggagttcaa tggcacgatc |
| 94021 | ttggctcact gcaacctctg cctcccgggt ttaagcaatt cttgtgcctt agcctcctga |
| 94081 | gtagcbgggc tacaggcgtc cgtcaccaca ctcagctaat tttttttaat agagatgggg |
| 94141 | tttcaccatg ttggtcaggc tggtctcgaa ctcttgacct caggtgattc ctcggcccta |
| 94201 | gcctcccaaa atgctgggat tacaggcgtg agccaccgtg cccgctattg accccatttg |
| 94261 | ttgatgctta gggatgaaag tggaaactcc actgctaaaa cagagaatat tacttgagtc |
| 94321 | attcctctgt catatatggg atgtttgcca gcatggaaca ttcacagaaa ttataactgt |
| 94381 | aaaggcccta gcaatgattt aatgccgtct cttcacatta tagttgggga agctcaaact |
| 94441 | gtgacagggt cagggacagg taaaattcac aaagttggtt tacagcagaa ctgtgctaca |
| 94501 | gttatctgga ttcagatcca agtcccctcc aaatgaatca caaccacttc tacctttata |
| 94561 | attccaggcc atgaaagatc actgttttag tctgcgtggt gcagtggaac agatagacct |
| 94621 | cggtttgaat ctcagctcta ctgtttacta gacatgaaat ggggaaatct aaaatgagta |
| 94681 | agtatacaca gaaacacaca ttacatacat atatgtaatg ttcaggatca ttacactgaa |
| 94741 | ctttagggct tagtactaat gtacaatcaa ggttgtgtta gtactgagca gtatatcttt |
| 94801 | tattgttgaa gtccaataag aacagagcag atatcaacct aatttgtaag tgatgcattt |
| 94861 | tcctaaacta aatgcccaat aatggaatgg gctagaatat tgcaggacag ggctgtgaat |
| 94921 | ggaggaaagc agttggaaaa tcaatccttt taaaggcaca aaaggatgaa tgaaccacat |
| 94981 | tcccaagaac ctgctcccac ccactgctgt tcccaccccc gctgtagttg gtagcatgtt |
| 95041 | cacaaggaag gagctggcta gctttcatgg gagccacatc atccatcaaa tcacattagt |
| 95101 | gttgttgatt tgaatgttgt ttcctttgta ggtctgcttg ctttgacttt gtaaaattgt |
| 95161 | ttagtcttgc cattattata taaggactat aatcataagg catttatatt tcattttgta |
| 95221 | ttatgtattt aagcaaaatt aagttaaaaa ttagacagag atatatagga tgatttttcc |
| 95281 | ttcaagagtt attcatttat ttccttttgg atatgaaaaa cgcttatgca gtcctgtatg |
| 95341 | tggtctctct ctctttttat ttttatttat ttattttttg agatggagtc ttgctctgtt |
| 95401 | gcgcaggctg gagtgcaggg gtgctatctc ggctcactga aacctctgcc tcctgggttt |
| 95461 | aaatgattct cctgcttcag cctcctgagt agcttggatt acaggtgcca gccaccatgt |
| 95521 | ccggctaatt tttttgtatt tttagtagag acggggtttc accatgttgg ccaggctggt |
| 95581 | cttgatcctg acctcaagtg atccgcttgc ctcagcctcc caaaatgctg ggattacagg |
| 95641 | catgagccat tgtgcctggc ctgtggtctc tttttaaatg tttgagattt tctctgacca |
| 95701 | tcacatgatg gcagccgcaa gttgtaaata tattaaatga gataatacca aatggcataa |
| 95761 | gaccactgtg aattcagtga tgttccaaaa taacttgtat tttattcatc acacaggcat |
| 95821 | ttatataaat taaaaaataa tagcacctga aaacatgaaa ataattgtat tcaaggttat |
| 95881 | ttactgcagc atgattttta agagaaaaga ctggaaacaa cccaaatgtc cactaatcga |
| 95941 | gactgtgtga acccaagatg ttatagctgc atcacacagc tgagagaagg aataagatct |
| 96001 | atttctactc actgccagtg gtaatctcca ggaagcgctg ttaagtgaaa gagcaaacta |
| 96061 | gagaaaatgt gaatagtggt taccatttac ccaagaagtc tatgtgtggt gtgtgtgtgt |
| 96121 | gtgtgtgtgt gtgtgtgtgt gtgtgtgtgt atgtgtgatt tctagaggga cagaggaaaa |
| 96181 | aggtgaagac aaggatagga gcgagacttc tctgggtaca tctttttttt tttttttttt |
| 96241 | ttttttgaga caaagtctca ttttgttacc caggctggag tgcagtcgct tgatctcagc |
| 96301 | ttactgcaac ctctgcctcc ggggttcaag tgactctctt gcctcagcct ctcgagttgc |
| 96361 | tgggattaca ggtgcccacc accacgcccg gctaattttt gtatttttag tggagactgg |
| 96421 | gtttcaccat gttggccagg ctggtctcct gaccttaagt gatccaccta ccatggcctc |
| 96481 | ccaaagtgct gggattatag gtgtgaacta ccatgcctgc ctgtctggat ctctttttag |
| 96541 | tacatttgac tttgaagcca agttagtatt ttccataatt aagaaaaaaa tagaataaaa |
| 96601 | aaggaacaat ttctaaaaag gaagtcaaaa gaaacacatt aaactaactg taagttgagt |
| 96661 | tggtagcata accacacaga cagaaattat ttcaagtgac tttaacacat actgatgtga |
| 96721 | ctgtgcatcc ctatcgagat agaaataaag gtaaatataa ataaaggtaa aaataagcaa |
| 96781 | gatataaata aagttaaaat gaactgcaac aatcctgaat agttttcaat aattatatca |
| 96841 | taaatagtaa ttttggtatt gttattttaa aacagttata tttataatac atatgttaat |
| 96901 | gtaatcatgt ttatgcttat cataacatat ggatgcacag ctattaggat aatgtataaa |
| 96961 | catgattatg ttaatgccat tagggaccaa gatttcagag aaaaacaaag aaaaatataa |
| 97021 | aattaaagaa gccagtaaaa atgttatctt aaatttaaac tggaaacctc agtataaact |
| 97081 | cataatgtgt tttcttttaa aaaattattt tcaaattcag tctcctgaat agaactcgca |
| 97141 | acaattacca agccagtagc aacgacaata aacagcccta gtgcccagat ggtgctctcc |
| 97201 | aaatgccatt ttccactaaa aggaatgagg gctccttgga gaattggatg ttttcagata |
| 97261 | cagagcagaa cacatacaaa aagttcctgg aacatttgtc atactgaaaa gcagatacac |
| 97321 | tatgaaagac tatagtaatt gtatcaaaag aactcaggag ccaccatgaa gaggccccca |
| 97381 | gtgtccagaa atggaataat gtgaacatta agaataataa atcttaattt gtcaagatac |
| 97441 | aaatttaatc atatcgtaga tactcatagc agcattattt aaaataatga aatattagac |
| 97501 | acaacttaaa tattcaaaac tagcagtgtg cccttatata tagaaatgtc atttaaacct |
| 97561 | tgtagaagag aagtttatga tttaggaaga tgtttacaat atgctgtaaa acacacacaa |
| 97621 | caagtcataa aatattatgc atgcagtata ttccagttta ttttaaatga tctaggtata |
| 97681 | aatatccata aaaagaaaac tgaatgaata aatggtgaaa tgtgaatagt tgctatatct |
| 97741 | gtgtggcagg attacaggtg gttgtattct agtttctctt tatgttccaa tcttctacag |
| 97801 | ttagcatgta taattttata tttttttaat acaaaatatg ttatttaact agtatttact |
| 97861 | aaacaaaaaa ttaaaaatta atatatttta taaaaacaaa agcatatgaa tagaaaagta |
| 97921 | cattattttt taggttatag acaatgcttg tacctgtgtt tagggatttg atcctccagg |
| 97981 | taggaaacta cagatctaat ttaacttatt taattaatta attaattaat taatttattt |
| 98041 | atttatttac tttgagacag agtctcgctc tgtcaccagg ctggagtgca gtggtgtgat |
| 98101 | ctcggctcac tgcaacctcc acctcctggg ttcaagtgat tctcctgcct cagcctcctg |
| 98161 | agcagctggg actacaggca tgtgccacca tgcccagcta atgtttgtat ttttagtaga |
| 98221 | gacggggttt caccacgttg gccaggatag tctcaatctc ttgaccttgg aatccaccca |
| 98281 | cctcaccctc ccaaagtgct aggattacag gcgggagcca ctgtgcctgg cctaatttaa |
| 98341 | aaaattttta agtcaccaaa aagcaaagtg acatgctcaa gtccattatt gccaagcaaa |
| 98401 | gaaaaaaatg tcaaaagtac aaaatgtggt tatgttttaa aaattagatt attattatta |
| 98461 | agataggatc tggctctgtt gcccagacgg gagtgcagtg gtgtactcat ggctcactga |
| 98521 | agccttgact tcttgggctc aaatgatcct ttcacctcag cctcctgagt agccgggact |
| 98581 | ataaggcagg caccaccatg cctggctaat taaaaaaaga attttttttt tgtagcaatg |
| 98641 | aggtctcact atgttgtcca ggctgatctt gaatgcctgg actcaagtga tcctcctacc |
| 98701 | tcagcctctc aaagtgctgg cattacagtc ctgaaccacc atacctagcc agatttctat |
| 98761 | tactttgaca tggtcctgga tacttcatct atgtgtgcaa aatttcgtac ttatctctta |
| 98821 | aatctggaga aaaatggcca aatttggtat agttgaattt atcatggggt gattaataag |
| 98881 | tgactaaaga ttgaccttca aggatctgaa gatctgacca gtgtagattt atagtaatta |
| 98941 | tagtaattac atggtacctc caggtatttt ctatacattg ccgtagttag ctatgaaagg |
| 99001 | atgtaaagaa taaaggcaat tgactatttt aaagaaatta acctcagaag gagattcttc |
| 99061 | acctaaatgt ccctgcaaat attattgctt tatgctctga gttagctgaa aatgagttta |
| 99121 | ctatgcctgg tgtaaacact tgcaccaccc acctgtgtca aagaactttt cactaactgc |
| 99181 | atcatgactg attgacgtca gtctacttgg caaccaagtg agccacttag aagaaggctc |
| 99241 | cccttatgag gttaatttct ttaaaatggt gaattgccct tattccttat gtcttttcat |
| 99301 | aactgattac acaaatgtat agaaaatacc tcttgagata caatacaatc tgtaactagt |
| 99361 | catcagagaa taacacgtta ttgtaacaca gatttagctt ggccataaag ggcatattta |
| 99421 | catacacatt gaaaacaact ttaataaaat gaaaaacata tatccttgga ttcttagcaa |
| 99481 | tttaacagca atgctctcag gggatcaagc tactatttat tgactccccc tgtgtgcctt |
| 99541 | acacgtaatt actgaccctc acaacatccc tgggagttag tgtcttacag gagacctttg |
| 99601 | acatttgtga gaggacatcg tgaatatttt ccaagtcctc aatccaaaag tgtggacata |
| 99661 | gctgttggtt ttcctgaatt gtatttcttt caagaatcaa atgttgcttg tgaatattct |
| 99721 | catgtaagat taaaaaaaat cttttcctga ctcttagaac tttagtttta tctctgcttt |
| 99781 | gagagccact ttaataagca aactaaaatc actctacaga aaggctgtac agcaaatgag |
| 99841 | tagagaacaa aagctggcta aactgggcag tcagctaaga tcactcagtg tctgagaatt |
| 99901 | tgttctagaa aatcaccctc cgagaatcat gcagattgtg cttaggcttc cttcatatat |
| 99961 | tcatactctc ctcgggtgaa cataaattgg cacatttata aggaaatttt attattatat |
| 100021 | actcaaatat ttttaaacat ataaatagtt tgtcttgtta attctacctc ttggaatcta |
| 100081 | tcctaaggaa aataatcata agatatctgc aaagaattat ttgaatgttg ataataattt |
| 100141 | tatctataac aataaaaatt tgcaaggtgg cagatatcag tttatgtctc tgagctccaa |
| 100201 | atccaccctt gtttatcttg ctttgtgtga atggagctgg accctgtgga tctttctctt |
| 100261 | ttgcaaattt gggggatatt caactttgtc aattgagggc actggagaga ccctgcaatg |
| 100321 | gaatagaggc tccaggtgca cctacaggcc atctttccct accagcagcg atttcagagt |
| 100381 | aacttcagcc tgtccatacc ctccagcaat ggtgggtcac ttctatagat gaactctggc |
| 100441 | ccacccgcag caccaagcaa aggaaggtca ctcctgtaca ccagcttcca accactctca |
| 100501 | tccaccagtg atgatagatt gcttttatgg atcaatgcca gcctttgcac tcagtcgttt |
| 100561 | ctttgttcct gcaggctgcc tgtggtagtt actatctctc tgaagaggtc tgaatctcag |
| 100621 | ccttggggca aggagggctt gtcctcatcc ttagtgcctt tactgttctt cttcctcagc |
| 100681 | ctagaaataa tagctccttt taaaattctt gatgcctggg cctcttagag tcctccttta |
| 100741 | tatatctttt agtaggtaat ttttggaaca ttaaattttc cctgttcaaa taattatcat |
| 100801 | agtttttgtc ttccacttgg accttgaatg atacaaaagt taaaggctca acaataccag |
| 100861 | acttgttaaa taagtaggga gctatccaca cactggcata aaattgtttt gattatctat |
| 100921 | tattcccttg gtgaggtgac ccagaccctt gtttgaaagc gttttattgg ctgggtgtgg |
| 100981 | tggttcatgc ctgtaatccc agcactttgg gaggctgagg tgggcagatc acctgaggtc |
| 101041 | aggagtttaa gaccagcctg gccaatgtgg tgaaacccca tctctactca aaagtacaaa |
| 101101 | aattagctgg gtgtggtggc gggtgcctgt aatcccagct actcgggagg ctgaggcagg |
| 101161 | agaatcactt gaacccgggt gtcggaggtt gcagggagcc gagattgtgc cactgcactc |
| 101221 | cagcctgggt gacagagcga gactccatct caaaaaaaaa aacaaacaaa agaaaaacaa |
| 101281 | caacaacaac aaaagaaaaa acaaaaatga aagaaagggt tttatttgtc ttctctctct |
| 101341 | ctccctgttt tttttttttt tctttttttt ttttttgtct catctttgct gtaattgctc |
| 101401 | atttgctagg gtcattggac acagagatac caagatgtct tcagaaatca tgaatattcc |
| 101461 | ccaagtcctc tatcaaaaag tgtggacaga gctgtcggtt ttcctggggt ttagacatgt |
| 101521 | tctttcttgc ttctgttgtg tagtaaccct agctcctcaa ggaaatcgtg gtctatgggc |
| 101581 | tctgccaaac agtaattcct ttctttgctt gctggctcac tggcttgagg aactcaaagt |
| 101641 | aatcctttgg taatttcagc ttccaggtca gtggggacat taatgtgttt cttggtagaa |
| 101701 | atgtcactct actccaagaa ataagatctt taatccagaa aacctgagtt ttcagggaca |
| 101761 | gaatgcacaa acttttatgg tcattcactg ggagttctaa tgagcaggtt cgatcatatt |
| 101821 | tctattcttt agttcctcga cccatgcatt ttagctactg ggcggcgaac catgtatcag |
| 101881 | ctgttagtgc tgtgtgtatt ccatatcatg aaggtaattt cccaggccca caacgttttc |
| 101941 | ttcccaagct ggtgccgtaa ctgagccttt accaggctat tccatcattt taggtgtttc |
| 102001 | tagggtatag agtaaatgat aagaccagtg actcccatga taaatgtgca ccatggtgaa |
| 102061 | cttcctttgc tgtaaaatgt catcttggtc ctagtctata tcgtaagggg caccatactg |
| 102121 | atagaccagg tactggtgag cccttggatg gtggtactgg tagaggaact gcaggaagaa |
| 102181 | aaggtaaatc catactcaga atatgtgttg attctggtaa ggacacgttg cttttccctc |
| 102241 | caagacagat ggttccgata gatggttgtg tagctaccct ccctgaggaa cggtactatg |
| 102301 | tcaaggtctt ggcatcagat tccgtggctg acaggttgga aatttagcat tgacagtagc |
| 102361 | tgaattatct tttgtaagtg ggagctcaaa tatgaccatg catagcacca tagtgattgc |
| 102421 | agcactagag acactattta tgggctttcc tgcacaagaa ctggggtggc taaagacggc |
| 102481 | ccggctaaaa ggcaggatgg atcatcctga gcacatggtt gttgagagtc ttctttgcag |
| 102541 | tcgggcggta gtgtgagatc tgctttgtgt ttatttccgt agggttgtcc acatacattt |
| 102601 | ttttccccac accttttctc tgagcttcaa atcttttttt cttgtcctgt cgaaatggcc |
| 102661 | aagccattca ctactgctta gaagccagtg catacttaag gatctggtct tttcttccat |
| 102721 | atgaaatgaa taactaagtg tgttgctcat agttttcccc actgggagga tttcccttta |
| 102781 | cctctcttct ttggggccac ctcaagtgag gatgaaatgc aacagcagac tatctctggc |
| 102841 | tagaggcaat aaatcacact agcccaatta tgcaccaggc ctgagctgtt tcctccacta |
| 102901 | ctgattgttt gtaggtagca gttgtgggag cttctgtgga acagattcag gtggtacagc |
| 102961 | tgctcaaact cataactcac ctgcaatgtg ttctgaaaat gtgtggctta actgcatcta |
| 103021 | ccctaaaaga acaagttggt gagtgatgag atgagtggga agtagtgagg atgacctgga |
| 103081 | ttttccattg aggaagtggc caagtcctgc ggacaaattg tacctaggaa aaggtacaaa |
| 103141 | aacaggagaa tggatcatgc gttcagtatt ggaactgatg actattaagt tgctgtgggc |
| 103201 | atctgaatat agatgttgtg atggttttaa aacatgtctg aaaattcttt gacactgttt |
| 103261 | ccatcaagag gtgatgtctt tgtctcctcc ccttgaatct aaatgggcct ttgtgacaga |
| 103321 | tctgatgaat agaatgcagg aatgtgatgt ccaaggctag tttagaaaag accatgtttg |
| 103381 | tttctctgga gacacttgct ctgggagcta ttagtgtcct tccaccaaag ccccccagga |
| 103441 | acacccctgc agctaaacaa attggattta ttgcttattg tagcaagaca gaacattcac |
| 103501 | cagggggaac cataggttat ctcagtaaga gggttttaga aagaacctat tataagattt |
| 103561 | gggctttggt tgggtgattt gggggaaggt ttaaggaatc aggggtggcc aggcacggtg |
| 103621 | gctcacgcct gtaatcccag cactttggga ggccgaggca ggtggatcac gagatcagga |
| 103681 | gatctagacc atcctgacta acacggtgaa accccgtctc tactaaaaat tcaaaaaatt |
| 103741 | agccgggcgt ggtggcgggt gcctatagtc ccagctactc aggaggctga ggcaggagaa |
| 103801 | tggcgtgaac ccaggaggcg gagcttgcag tgagctgaga tcgcgccact gtgctccagc |
| 103861 | ctgggtgaca gagcgagact ctgtctcaaa aaaaaaaaaa agaaaaaaaa aaaggaatca |
| 103921 | ggggtttggt ctagattgaa tgctctcaga aaatgggagt aattctaggg ttgtatattt |
| 103981 | tataggtggc atagtgacct tgttttgtct gtccttacac aaatgatgaa gtaggctgat |
| 104041 | tttgtttcat tttatcttgg tcgcagagta actttgtctg aggttggtgt tctataagat |
| 104101 | tgtatatgtc caacaagaga atgacatggc ttagctgtga atgtcagatc agttctggat |
| 104161 | atgagaggct gatattttct ttttgagctt tcagttgtca tgtaaggagt gtggctacca |
| 104221 | tgaggccact gcatagagtg attgtatacc tatatataag agagatggtt tggagttcaa |
| 104281 | gcccccagct agtcaagtct tcccagccca ggagacagat gttcgaatga aggagactta |
| 104341 | ggtaatttca gccccagtca ctgtctgacc acaactgcat gagagaccct gagggagaac |
| 104401 | tgctttgctg aacccagtca acctccagat ttgtaagtaa aataaatggt tatcattgat |
| 104461 | tgaagtcact gttctagggt ggtttttcct acagtaatgt cattgataca ggtgtctggc |
| 104521 | aggcagttgg ctctagaggt ctggagctaa ggagaaagat ctggcttgga gatgtggatc |
| 104581 | aggaagtcct tttacaagga gaaaagtgct tgaagtcatt tgagtggatg acatcacctg |
| 104641 | aaagataata tacattgaga aagaaaaaaa taaaaagcca acaatagcac ctttagtaac |
| 104701 | aacactgaag agagcaggaa gaaataaaga aacatttgca aggagacaga gaaggagtga |
| 104761 | ataaggtgat caaatggcat ccatccaaga gatgatgtcc aggaagccaa gaatgagagt |
| 104821 | atttcagaaa gaagagtgtt gtccacagag ttaaatgcta tggagagaga tggagtaaga |
| 104881 | caagattgat tagcaccaat gagctttcat tgttgacaaa tgcagtttga gggaaactgg |
| 104941 | gggctaggga gtgatggggc agagaccaga atgcagtggg gtaaggaggg gtaagaagat |
| 105001 | gagaagaatg ccaaatctgt gctgtttgcc acatcatggg ccctctctcc atactattta |
| 105061 | tgtgtgtata atttttttct acaatttaat aatgcattaa accatcacat cacatcacat |
| 105121 | cacaaaccca acaattccac aatagcatgg gtagatcata ccctggtgta tttttaatgt |
| 105181 | tatactttaa ccggtagttt tgagtcataa atgtactact atgttgttgg tttttttttt |
| 105241 | ttttttgggt ctgtcattat agtaataact ttaaatctct tattgtttat ttctcttgca |
| 105301 | attaaaatag ttcatcattt aaaatagtac ccagtgttca gtgtccattg gtgccttgta |
| 105361 | attacttcag aaaaacagtc tcagtttaaa agttataaac tcttaatgcc tctctgcagg |
| 105421 | aggaatagct tccttctgcc agagcggtct ggggagcatt attctagtca taccctgcta |
| 105481 | attcattggc ttgattgtca ggggtcctga tctagttcac cctcctggga ggctggtgtg |
| 105541 | ggaccctgct tatcccagtg ggggtcaagg cctccttgac aacagtgaat gggttgctag |
| 105601 | gaggctgcgg aaagaaattt ctctagttcc atccacaagt agatcccact gcagtggcct |
| 105661 | catttggctc ctctaatcct ctctttccca ccttctttca gggcaagtct caccctattt |
| 105721 | tcttcattac ttgcttgtgg tttccttgcc ggagatgctt ggaggcaagc attatgacaa |
| 105781 | ggatgggcgc gggtgggagt aaggccttta aacgaagagc tacatttcaa agaacttcaa |
| 105841 | atgtgtattt caaatgtcac ttccaaatga ggttgtacac acagacagaa acgcaattaa |
| 105901 | cagatttgga agaggccacg tagcatgcat ttgaaactcc tgttgcattc cttggctatt |
| 105961 | cttgtctgtg gcatcttttt aacaaatcct gtagttacct tgtaaatacc atcatagaat |
| 106021 | atattgtatt taaaaaacat tctttactca gatatgaaag ttgaaaaggt actattattt |
| 106081 | ttaaaaatct gtattggatt tttttttttt catattttag gatgccagaa gcctcaaaaa |
| 106141 | tggaaaaccc cctgtgcttc acatctgaaa atctctgctg ggggcagcaa ctttgagcct |
| 106201 | gtggggaagg aactgtccac gtggagtggt ctggtgaatg cttaaggagc tgcagaaggg |
| 106261 | aaggtagtga ggagacagtt aaatattgag cacgttttgg tgcctagcat tgtactggcc |
| 106321 | tttcacatgc tatgtttaat cacgcaggat tgtatggaat gggattgtac agaatgaggc |
| 106381 | ctgggtttgc taaatcctgg gggagattgt ctaagcbgtc acttgaaggg aaaactgtag |
| 106441 | catgbgggac ttaaaaacag cttgggatgg agtttgaggt agggcgagag ggaaaaagat |
| 106501 | cttggcagat acattctgaa caaatacttg tgtcatcctc aactgtatag tttttatgac |
| 106561 | ttttcctgtg ttcatctggt tggcagatga atgcaaaatg ccttttagct tcatggaagg |
| 106621 | cacacatgaa tcatggctat gtgataaaca gaagaagcca cgtttcggag ggtttctggt |
| 106681 | gtgtgtacgt gtgcacttgc atgtgtgtgg gcacaccctt gtgccaaatg gaccaccagt |
| 106741 | ttaaatcgat tttcaacagg gcagatttca tatgcttctt tagtgtggcc caagtcaatc |
| 106801 | tatttttaga aggggaaagt agagcatgtt ctgaaagttc ttttcttctg ccatcaggga |
| 106861 | ttgtagtact ttttatttat agtaatttca ggtctctaaa tacttcaagc aaaattaatg |
| 106921 | aggcaaacaa aaaaaattag gtaccagata attggatcat gtgttatttt atagagagct |
| 106981 | ttatttatct tgcctttgga aagtataatt tgtatttttc aaaattatga ttagatctaa |
| 107041 | ttctttttca agatttactt aaaataaata tttctgccta ggcacaagac tgtgtatagc |
| 107101 | acaactggcc ttgtattatt gacttgatga tgcaaagaaa aatatataag aatcagtttt |
| 107161 | ttttaaatgt gttaatattt atcataatgg aaatagtctt actaattagc aataactaga |
| 107221 | aaaatgtcta ctatttattg agccactact atatgccata ttctttacct atgttcattt |
| 107281 | tctcttctat attcaaaata atcctataag gtgtgcttta tgatcaccat ttgttccaat |
| 107341 | agggtctcaa ggacgttagg taattattcc tatgacattg agcctatgaa tggcagagct |
| 107401 | gggagtttga acctaagtct gatgactctg aagcttatag tctttctata agcttcttca |
| 107461 | gtctgaagct gcccctcaaa gtcatggcaa taactgagaa tagcattcag ttttacagct |
| 107521 | aggaaggtag ttgtagttgt gtttattatt caatactgtc catattcaaa gaaccattca |
| 107581 | ttgagcttct attttactcc agtcacggtg ataaataatg cagaatctag agatcaaaga |
| 107641 | taaaatggcc ttgccctcaa ggaacccatg agctagttgg gaaaactaat aggaatatta |
| 107701 | atgattataa tgcagaatgt taggtgcaat gagagaggta acaaggtact actggggcct |
| 107761 | gacaaaggcc accgtttctg tgtgtgtgtg tgtttatgtg tgtttgggaa aaggcaaaag |
| 107821 | acacttgaga taaagggaac aacatagaca aaatcaaagg cttgagagca tataacttat |
| 107881 | atgagacata gcaaattgtt tgctttaact agaaaatagt ggttatgtgt ggcagactat |
| 107941 | gagaaatgag aatgtaagtc cttgagaatg ataactttag aaattctaca tttgggatga |
| 108001 | tcataacctt ttaaaattgt ctcaatatga tagatatttt ccttctgttt tggggtatct |
| 108061 | gtctttttat tttttatttt tttgaggtgg agtctcactc tgtcacctag gctggagtgc |
| 108121 | aatggcgtga tcttggctca ctgcaacctt cacctcctgg gttcaagcaa ttctcctgcc |
| 108181 | tcagcctcct gagtagctgg gattacaggc gcctgccacc atgcctggct attttttgta |
| 108241 | tttttaatag agatggggtt tctccatctt ggccaggctg gtctcgaact cctgacctca |
| 108301 | gatgatccac ctgcctcagc ctcccaaagt gctgagatta caggcatgag ctaccacgcc |
| 108361 | tggcctgggg tgtccatctt ttaaagacac actgtaagaa tcataatatt ttcccaaaga |
| 108421 | agaaatgtaa ttagtacaca attacatgtt aaaactaggt gttattggta attaaaattt |
| 108481 | aaaaaaatta acccaaccaa ataataaaac taatgaaaat atttaatatt ggtaagaaca |
| 108541 | agatgcgata agcatatttg tgttttgctg attggtctat aaagtggttc aaaacttttg |
| 108601 | taaactatat ataagctatt ttaaatgttt atgccctttg attcaatagc actaggtttg |
| 108661 | gtactttata ataggaaagt tattagaaat atttactgca gggtttcttc aatgatgagt |
| 108721 | tgtaagaatc agcctaagta gctatggtac atcactttga tgcaacttaa tgtgtcatta |
| 108781 | aaaatcatgt ttatgaataa cattccagat gcatttaata caattctagc tgaaaaaaag |
| 108841 | atactgaaac attttgtgtt tatatatgtc tgcatatgta tctatatata tccctctccc |
| 108901 | tgtcaatcta taaattaaaa actatacatt aaaaactgca aattaaaact atatactaaa |
| 108961 | gaaagaaact ggaaagaaat ccacagaatg ttaataatca tggtcattgt gttgagtggt |
| 109021 | aggactatgg gtaatttttt ctttctattt ttatacattt ttcacttttt tttttttttt |
| 109081 | caatttgagg cagagtctca ctctgtcacc caggctggag tgcagtggaa cgatcttggc |
| 109141 | ttactgcagc ctctgcctcc gggttcaatc aattattcta cctcagcctc cagagtagct |
| 109201 | ggaattacag gtgcacacca tcacgcccgg ctaatttttg tatttttggt agagacaggg |
| 109261 | tttcactatg ttggtcaggc tagtctcaaa gtcctcccaa agtgctgggc ctccaaaagt |
| 109321 | gcbgggattt ggcctcccaa aatgctggga ttacaggtgt gagcaccaca cccggcccat |
| 109381 | ttttcacatt tttaatgatg tttttaactt taaaagtaaa aatttgtaat ttaaaatttg |
| 109441 | tttaactttt acaaataaaa attacaaatg caaattaaaa ttgctatata tttttgttac |
| 109501 | tggacaagaa aagatgcatt atagagtctc gttttatttt gttttctgat aaatctattg |
| 109561 | ctggggagca tgtgcgtaat tcgttttaaa aattctcagt gttaactaga tttcttttgg |
| 109621 | gatagctctg ctgaactgga atgagatctt cttccccaga gccacttgct agtttgattt |
| 109681 | tctagaaaga gagaaattat agaaagctac aatctacaaa ttgctatgta ctacagtcta |
| 109741 | ctaaatagta taatacttta ttttgcaaac tatatctctc agagtacaag gctccacagc |
| 109801 | tagtcttgca ccaaatatat gaggttcttg aatagtcatt ttctcaaagt tctcatctct |
| 109861 | ttacaaagca agttgagggg ggaagcagag agagcactct ggtgcttcca aaagcatcag |
| 109921 | ccatccacac tctgcctatt gaaaacacca aatctgagca tgtgtgtcat tgtccaggaa |
| 109981 | taaacaatca caaaaaggca tcaatgaggc ctataaaaac aagagcgcat gcaagaagat |
| 110041 | gggattgtta tgaaacagga gcacaaggct actaaaagag ggcaagccta aaggaaaagt |
| 110101 | caccagggtg agatggaaat agatatagga agaatagtga acggtggcaa ggaagtaaag |
| 110161 | gaatacaaaa gtaataaaat atccattgca ttagtccgtc cttgcattgc tgtaaagaaa |
| 110221 | tacctgagac tggataattt ataaagaaaa gggtttattt ggtttatggt tctgcaggct |
| 110281 | gtatgagaag catggtgcca gcatctgctc ctggtgaggc ctcaggaagc ttctattcat |
| 110341 | ggaggaaggt gaagggggag ctagcatatc acatggtgag agatggagtg agagagagag |
| 110401 | agaaagagag agaggagatg ccagtctttt aaataaccag atcttatgtg aactcagagt |
| 110461 | aagaactcac ttatcatgag gacagcatca agacattcat gaggaatctg accccatgac |
| 110521 | ccaaacacct cccactaggc ccacctccaa catgggcagt cacatttcaa cataagactt |
| 110581 | ggagaggaca gaacatccaa accatatcac ccacattgag ggagcaagga gcagactcca |
| 110641 | tactgcagaa cacacatggg aaacaaactt gagaagtttc cccagggcat agggtaagag |
| 110701 | gacgaggggc tggaaaaaga aaggtaatgg atatagagga cagagaatgg agttcgaaga |
| 110761 | tacatctaag aactttacat attcctaagg aacccacaaa gataaaacaa acagagcaag |
| 110821 | tatgaaagat gtaatagaag caaatgaccc tgcattgaaa atgatcaaag tatgcagatt |
| 110881 | ggaaaggctc acaaattcca ataaaaagaa ttgaaaacaa aaaaaaatgc tgcaataaca |
| 110941 | ttctttaaac tatattaatt gtaaggtttt tacaaaaata agtactcagg cagtagtaaa |
| 111001 | aagtttgcga ataaagaaac caaaattaaa ctctaacttt tcttatgtaa taccaaattc |
| 111061 | cagaacttgg aaacatatct gcatgcttta ccagggacca gattatgagg cagttattta |
| 111121 | agattagaaa agttctcttt cattggtaaa ggcaacagaa agatatattt tagaggtaga |
| 111181 | agctctctaa catagacact attctgccaa aaaaaaaaaa taagataaat gtaacaaaat |
| 111241 | aaaatgagct tcaaggtgta ttttggtcac tgagagatga ttcaaaagaa gactttgaac |
| 111301 | aatgagaatg ttaacattta aaagaatagg tggaattttt ccttactcag tttgttcaat |
| 111361 | aaaatcatgg gtccactgta acctgatata tcttgttgct taaagggaaa gcttggccct |
| 111421 | accttttctg ctttcttctg tgttcatggt gtagaaaatg tatttttgtt aaccaattat |
| 111481 | ttacttcaca cttgaggtag actttgttgc catgttggat ggctattttg ttaggtcaaa |
| 111541 | cagttttctg ccacatgcct atgtgatttt aaataatgta gccctcaccc tgagcttctg |
| 111601 | ctatagggac cacatgcttt gtggtaacca ggtgctgcca aaattggatg atagcaggct |
| 111661 | cctgacccat gcctgtcaag tcataggtgg ttaaggtcat gggagttgga gtcagactgt |
| 111721 | ctggttccaa atccaaggtc taccagttga ttttgacctt aattttctta ttctgtaaaa |
| 111781 | tgagtttatt gattgttccc actcagttcg tttgaggtga ggattaaatg agttaatacc |
| 111841 | tgtaactttt tgactggcac aagacacaat cctattacat gttctgaaaa aaaattctct |
| 111901 | acatgaacca aaggaatgaa ttctatttcc tccactgcta ctacaactca acacttctaa |
| 111961 | tcaccaaatg tgtggaggtt tttctacatc agtttgtcag acacctacta tgtgtcctat |
| 112021 | aattcagttc agttctgaca ccatctacat ggaaacaaca tccgatccca tggactaaaa |
| 112081 | gctcagtctc ataagactgt ctataatggc tcaagtgggt tcttcttgcc tgctgcacag |
| 112141 | aaaaaccaat acatcaaggc agtggtgttg cagtagagaa agcatttgat aatcacaagg |
| 112201 | ccagccaagt ggaagcatgg gagataattc ttaaatctgc ctccccaaga gctcaggggc |
| 112261 | tagggttttt aaggataatt tggcagacag gggactaggg aacgagtgct gctgatgagt |
| 112321 | tgtggatgaa atcataggag tatccaaact gtcttcgcat gctgagtcag tttctgggtg |
| 112381 | gaagtttctg cgtgggagtc aatttcttgg catgagtcac aggtccgggt ggagtcagtt |
| 112441 | agttgccaga atgcgaaagt ttaaaaatat ctcagagacc aatcttagat tttgacaata |
| 112501 | gtgattttat ctataggagc aagaggggaa gttaaaaatc ttgtgacctc tggctacatg |
| 112561 | attcttgagc agtaactatt atagaaaggt aagctaggga acaatgtctg gttatcattt |
| 112621 | aattacacct acatcttagc agaatttaag ttcttctcat aagcccaacc ttgtggactt |
| 112681 | tcattagtct tacaaaggca gtttcagtcc cccaccaagg agggcattag tttcaggtag |
| 112741 | ggactgttat catctttgtt ttaaagttaa aaaaggcagt tagcttgtga tgttagaaga |
| 112801 | aagatgtcag atttctctca ctgtcataat ttttgcaaag gtggtttcat ggccccactt |
| 112861 | gaaatgagaa tgggaagtcc aggttttcac ctgtgcttta gactgggtgg agataaatta |
| 112921 | aaggttcaca ggacctcctc ctcagatttg accatttgct agaatggttc acggaactca |
| 112981 | gaaaaacagt ttacttattt gattaccagt ttactttaaa aggaaattac aactcaggaa |
| 113041 | gagccagatg gaagagaggc atagggcaag gtatgtggga aggagcatga agcttccgtg |
| 113101 | cattcttggg ctcatcaccc tctcagcacc tccatgtgtt cagcaacctg gaagctcttc |
| 113161 | gaaaaatgtt cttaagccag gtgtggtggc tcacacctgt aatccccgca cattgggagg |
| 113221 | ctgaggtggg tggatcatct gaggtcagca gtttaagacc agcctggcca atgtggcgaa |
| 113281 | accccgtctt tactaaaaag agattagcca ggtgtggtgg cacatgcctg taatcctagc |
| 113341 | tacttgggag actgaggcag gagaattgct tgaactcagg aggcagaggt tgcagtaagc |
| 113401 | caagattgtg tcactgtact ctggcctagg tgacagagca ggactccgtc tccaaaaaaa |
| 113461 | aaaaaagaaa gaaaagaaaa agaaaaatgt tccttcagat ttttatgagg gcttcattgc |
| 113521 | atatacatgc ccctccctgg aggttggtct ggaggttggg ctgaaagttc caactctaac |
| 113581 | cacatggttg atttccatgg caacagccct gcttatcatt agggacttcc caaaagtcac |
| 113641 | ctcattaaca caaactctgg tgtgactgaa taacaaaaga tgctcctgtt acctttattg |
| 113701 | ctcttagaaa attccaaggg ctttaggggc tttgtgccaa aaacaaacac tatatatatc |
| 113761 | ttattataaa tcacactatc tcacattatt attatttaaa atgataagtc agtcaagctc |
| 113821 | tcttttcttg agaatttgat ttgggaaact tcgagaatga ggcaaggagc agtgggaata |
| 113881 | ggagtttaaa gtaagcatag aaagcataga aagaagctag ggaatagatt aaggttgcaa |
| 113941 | ggagggataa caaatggaga ttaggaaata gtggagactg tgattaagca gagcctgtca |
| 114001 | ttaggcattt ctgatataaa cccagatgct gtcatatcac ctcactgaat gcttgccctg |
| 114061 | aaacacttgt tttagtgcag ataaattcat cacctaagga tggcttatcc tttctatggc |
| 114121 | cattgaactt aagagctcca ctaaatagga taactccctt tatgcbggga aatctaccaa |
| 114181 | aattacctgt gtcagaaggg taagaaaaga gaatccagga tccagaatgg gattgaagtg |
| 114241 | atctcccatt ctttttgtga tatttagact gccttctgcc tctccaatcg acagaataaa |
| 114301 | gaccaaactt ctacccgggt gggtagggct tatcctgagg atggagactt tctaagtata |
| 114361 | gatggtcaag aaactggtgc acatttaagt tagacgcatc tctaaattgt ctgggcttag |
| 114421 | actattctaa gaatgaataa ttatgaaaat tgaggcagct cagagactca ttttctaaga |
| 114481 | tttcaatata tgattagcaa agtgctatag aaaaaaataa aatggcacag tttggcagtt |
| 114541 | ggcatcttgt atctttctgt cccaggtctt tcttgtgctt gtaattcttt cattgataaa |
| 114601 | gaactcagct atctatccag gcgtgatggc tcatgcctgt tatctcagca ctttgggagg |
| 114661 | ctgaggcggg aggatcactt gaggtcagga gttcgagacc agcctggcca ccatggtgaa |
| 114721 | acccacatgg tgaaacatct gtactaaaaa tacaaaagtt agcbgggtgt ggtggtggga |
| 114781 | gcctgcagtc tgagctactc aggaggctga ggcaggagaa ttcctcgaac ctgggacttg |
| 114841 | gaggttgcag agagccaaga ttgcgccact gcactccagc ctgggtgaca gagcaagact |
| 114901 | ccatctcaaa aaaaaaaaaa aaaaggattc agcaatgtgt ggttcttttg aatctatggt |
| 114961 | atgtattagc acatcacagc agagcagagg ttctcaacaa ttgattttat tccccagggg |
| 115021 | acatttggca atgtctggag acaattttgg aacttagagg ctgatgctat tttggaactt |
| 115081 | agaggcttag atgctattgg tgttgtgggc agatgacaag agagattgca aacgttctcc |
| 115141 | aattcagaag gcagcctcca caaaaagaat tattcagtcc aaagtgttaa tagtgctgtt |
| 115201 | attgagaaac catgccctag agaagcagcc tagaactttg agtttttcct tttttttttt |
| 115261 | tcaatcaact aatgagaata aatgtggagg agacgggcaa aaatgtcagc agccccgtat |
| 115321 | ggttttcaaa atcatttcag atatttgcta ttcaccagtg tcagccaatg tgtttatttt |
| 115381 | tgaaaggtca gaaatttcat agggccagat tactaaaatg gttattatta tttttttgtt |
| 115441 | ttcaagagct tatgttagtt atctagtata gagaagtcta ccatagagat ccctttgttc |
| 115501 | tgagtactgg actttttcac ttaccaaaac tctcctactc tatacgttct tcaccgtgat |
| 115561 | aatccaagaa gtctgaatcc tccccatggc tctcccctgt ttccatcccc agtcggtggg |
| 115621 | tttcccacag gtctccagtg tgctttggaa gtaggaactc ctaacagatt cacagtttga |
| 115681 | acatggtagt cctctgagct gttcagtatt ttaaatctct ctgcagttag ttataatcat |
| 115741 | tcaactcttt tagtcattct tctaatgtca gtatagataa ctaaaagatt gtaatattag |
| 115801 | aagacctatt tcccagagac cacaaagaca atctcatgga agtcccactg tagtaaatag |
| 115861 | agtctatttt ctacccagca tctggtagag taattcagac atgtgttttg acagtttgcc |
| 115921 | tgaaagtaac ttgacaaata attttactaa agaatcctag aaagtcaatg gcaacatttt |
| 115981 | gttcattcta agttgttcaa gccacgtcct tagatgtggt tgattcaaat ttcttcagaa |
| 116041 | ctggaaaagc acaagagaat tagtattcca gtaataagga atcatggatt gtggcagtct |
| 116101 | ttttaaaaca taaactagtt tagcacataa gtggtggcct agtacatttt aaagtggagt |
| 116161 | ccaagggaag tttcttgaat tgctgtagtt cttatggtgt tctgctgaag ccgtgtagat |
| 116221 | cttatggtgt tctgctgaag ccgtgtagat attgatcatg ctgcctccca ccaccacccc |
| 116281 | cttcattcca gatctgctca aagacaactt agaaggtcct atcctggtct cctgtctgaa |
| 116341 | acatatactt tctctcctcc cgcccccagc ctgcccatac tattccaatc cttctctacc |
| 116401 | tattattcct ttgttttgtt ttcaatataa aaatcttatc tatttcttta tacgttaatc |
| 116461 | atctatcact ccccacacac tacaatgtgt gcattcattc cctgagtaac tgaaacctag |
| 116521 | aggcccggga gtcatttttg aaacttcctt tccttggctt tctcctctaa ctgctcttcc |
| 116581 | aattcatcgg ctggatttga ctattctaat tccaaatctg tctggaactc gtgtacctct |
| 116641 | cgcagtgtcc actgcbgttg ttctggtctg gggcacaatc gacactcatg ctcaccgaac |
| 116701 | tgcagcagcc cccttcctgc tgcttatttt gaccacacct ctttttgaac ctattctgta |
| 116761 | caaaggagct ggaaagagtc agtaaaaata cagagcatag tgttcattgc cttgcttgga |
| 116821 | caggacctgc catatgtgtc ttctctatag tattggctga atgcataaac agcgagaaat |
| 116881 | aaatgtgtag agagtaaatt gatagaaagc tgccctcagt catctcccat ggagagggct |
| 116941 | agtgatgact ggcgaaggtc acagagctac tatgtgttag attttggact aaaatccagt |
| 117001 | ttttctgaga acggtcaaga gtttatttat agaaaacatg ctgctttatc tcttaattcc |
| 117061 | ttctcactcc ctgcaaatta gtggcatttt agtttactta aatgcacaat tatttttaat |
| 117121 | tttgattgaa aaattaatac tcatttactt ctgaaaacta tttctttacc gaagagcata |
| 117181 | gagaaataag ggagtaattt atcaacctac cataggtgga atttttactt tcatctttat |
| 117241 | actttcttct tcattattgt tttttttaaa aaacgtaagt atgcatttat ttttataatc |
| 117301 | aggaaatatg tgaagttata taaatgtgta atgaatttca ttactgggaa ctaactgctg |
| 117361 | gtgatatttt acacttgatt ttagccaaaa ggccgagaag cgatgctggt gatattctaa |
| 117421 | tgtactataa cttattctaa tatttcatgg gatttatttg caattagtgt gatataaatt |
| 117481 | tccataatgt atggaaagat atattagaag atgctaacca tgggcttaag aaagtagatt |
| 117541 | tcataatttg aaacacggcc cacacaatta ataaatataa gacatttggc actctacttc |
| 117601 | taatctcttg atttttttta tagcaactgt ctttaagaac cccagcatat tcatggattt |
| 117661 | tttagaaacc taattatttc ctagaaaaat cctttgaaaa aggaaaatta tatttgacag |
| 117721 | ttttctaaag attttctgtg cagaagttgt atctggatat acatataaag ccctgaagta |
| 117781 | atctatacat tttatcgctg gtcttcagat atgttggcag agtcgctcgt gaaatatatg |
| 117841 | cttgaaatta atgtaattac tctgacattt gttattttaa taagcattct tagctaatac |
| 117901 | atagaggaat acattaaagg aactctaaca ccaaaattat cctttcttaa ttaaataaga |
| 117961 | aggactgcaa ttatttaaaa agtgtatgtc aaatctaagt tgccttcaaa tgtaatttaa |
| 118021 | ttttctgtta cttacatttt aaaatggcca tagctttttt cgttgttcat ctttcttaat |
| 118081 | tctttaggca ctacttaccc cccaactcca gcaaccaaaa ttaatcctaa tttgcatcac |
| 118141 | aataatttaa aatagatcaa agaaccaatt tcatagccta attcaaaagc atcatgcgac |
| 118201 | caatatgtag tattaataaa acatatttct ggttttgcca atccaatttt ttttttaatt |
| 118261 | aagtgcttaa cacagtgccc agcacagaaa aagcacttga tacatgtttg gtaatgtctt |
| 118321 | tgtgactgtc atttttacta atgttgtata ttggtactac taaagtctaa tgggatgctt |
| 118381 | gtattatggt gtgataatat agttaggatg accacttgtc atggctggcc tgtgagaatc |
| 118441 | ctgtttaata tctattgttc cagttttctc caacttaaca ttttagcact caaaagtatg |
| 118501 | tgatgtggag gataaatttt atggtcgcca atcctttatt gtaaagaatc gtgatgcaaa |
| 118561 | ggctccagtg tagtcatcac tcatctcttg atttaggcat ctaagctatc caggcatcat |
| 118621 | catgggggtg agcagcctga atgacagtaa gctttccaag tatggggaca tgcacttaag |
| 118681 | ataggtatag ttattccagt gccaatctcc tcttacctgc tgtatgtcaa gaatcaggca |
| 118741 | acatttagga aatgaaacca caagtctttg tctctttttt taccctttat ctcaaaatgg |
| 118801 | tttttacttg ggatttgttt ttttgttttt tgttttttgt tttttttttt gagacggagt |
| 118861 | gccgctctgt cgcccaggct ggagtgcagt ggcgcgacct cggctcactg caagctctgc |
| 118921 | ctcccaggtt cacaccattc tcctgcctca cctcccgggt agcbgggact acaggcgccc |
| 118981 | gccactacgc ctggctaatt ttttgtattt ttagtagaga cagggtttca ctgtgttagc |
| 119041 | caggatgatc tcaatctcct gacctcatga tccacccgcc tcggcctccc aaagtgctgg |
| 119101 | gattacaggt gtgagccacg gtgcctggcc tttagttgag atttgattat gaatgaaaat |
| 119161 | gtaaagaaat ttgctagatg tgtccatctt ttcatgtggc agacaatatg gactagatat |
| 119221 | atggtggtca aaattatgtt tgtgggtata gcctaatagc ctaaaccaat gacacttatg |
| 119281 | tggcagtttt atttatttat tttttctttt taagacaggg cctcactctg ttgcccagtc |
| 119341 | tggaatgcag tggtgcaatc tcagctcact gaaacctccg cctcctgggt tcaaatgatc |
| 119401 | ctcctgccta aacctcctga gtagctgaga ttacaggcat gagccaccat gcccaactaa |
| 119461 | tttttgtatt tttagtagag atggtgcttt accatgttgg ctaggcttgt tttaactcct |
| 119521 | gacctcaagt gatccacctg ccttggcctc ccaaaatgct gggatagcag gcgtgagcca |
| 119581 | ctacgcccag cctcaccctg gactttatga caatgccaaa tatatgcact gtgtcaaaaa |
| 119641 | cagtcctctt cactgtcctc cagtagaaaa agctcaataa tgggtctaca aactcctgga |
| 119701 | tttagtccag gctatgtaat ttgtggaact tagtgccaaa agtagggtcc tttgttcaaa |
| 119761 | aattattaag aatttccaga tggcaacagt ggaacagtca atgaagcaca ggatccttct |
| 119821 | gtccccaagg tcctgaacaa ctgcagaaac cttgtgcccg tgcattcatc cctccttctt |
| 119881 | ggatccttta cctgttgatc tccttcgttt tctttccaga caacaattat ggacaactta |
| 119941 | ttattaatat atgtgagcca ctgtgttcac tatcggaaat aaaaatatgc ttataaggta |
| 120001 | gtcccaaatc tttttttttt tttttttttt tttttgagag acagtctcgc tctattgccc |
| 120061 | aggctggagt gcagtggtgc gatctcggct cactgcactc tccacctccc gggttcaagt |
| 120121 | gattctcctc ccgcagcctc ctgagtagct aggattacag gcgtgagcca ccactcccgg |
| 120181 | ctaatttttt gtatttttag tagagacagg gtttcatgat agtggccagg ctggtttcga |
| 120241 | actcctgacc tcatgatccg cctgccttgg cctcccaagg tgctgggatt acaggagtga |
| 120301 | gccactgtgc ctggccccag atcttagatt ttggtgatta aggacttcac gaagtagatg |
| 120361 | aaataacacc cagaatttgg ctggtttatt ttctccttaa gatgctcagc tgtgattttc |
| 120421 | tgtatattag tctcaagctg caaattctct attccaggat cttatgatct gtgtcctaac |
| 120481 | tacatattat agggccccag tggtccctat gcctctggtt ctattgtcat cacacacttc |
| 120541 | ctgtttcatt cctatagaca gatttattca tgactaggga acagattatg ctagtaaagg |
| 120601 | tgttggttaa catgagttta aggcaagaac ttaaacgtaa atatctctag ggattggaca |
| 120661 | ggcaatatgc cttaaatagt aaagctatca ggtagggatt gactgcatgc ctgcagagga |
| 120721 | aactgctgcc tatttagcca cagaagatta ttgccatgta aaaatgtgag tatggtttgt |
| 120781 | cacatatttt taaagaaaag tcagaaatcc aaaaatttgt atgaaatttt taaacatttt |
| 120841 | atattgtcaa ttcaattttt aaaaagactc aatataggca gagaaaaata actacagccc |
| 120901 | aaggttaagc caagaagtat tgtttttcta ctttttcttc gtagtcagat ttgttcagat |
| 120961 | ataattcaga tgtaataaat aaacccttta agttctatga gttttaacaa acacctacat |
| 121021 | tagtgtgaca atcactgtag acaagatata gaataattct ctcgtctccc ctccaaaatt |
| 121081 | ctctcaagcc tctttgtggt taattctcct agctcctgac aatcgctaat aaatttcctg |
| 121141 | tctatagttt tgccttttca agagtgtcaa ataaatggaa cactactgta tgtagccttt |
| 121201 | tgagacaggc ttctttcact gagcacaaat aatttgactt attcacgttg ttatatgtat |
| 121261 | ttagattgtt tctatctaat tttcaaaggt tttttccttc ctttcttctc ctttcctccc |
| 121321 | tccttccttc ctttcttttt ttcttcttcc tccttttatt ctgccttttt tctctccttc |
| 121381 | caactttccc gcttttttag atatcacatg tacagaagta gattaatctt gatgtttatt |
| 121441 | tcataacata ctctcatttg ggcccatgca tggcttgtat ttcaatagct ctattgagat |
| 121501 | aaaactcata tacattcaca tggagaaaca gccctgtgga gggaaagaaa ttgatatgca |
| 121561 | gtaaactaca catactcatt tacaacttga aaagtttgcc atatgtatac acaatgaaac |
| 121621 | catcaacact ttcaaggtaa taacatatct cagtttcctt gtgtctttat gttattcttc |
| 121681 | cctccttcca tctgatctgc tttgtgtcac tgcattttct agaattttat gtaaattaaa |
| 121741 | tcatatagca tgtgctcttt tttttgtctg gcttctttca tgcagcacaa ttattttgcc |
| 121801 | attcatatca gtttttgtgt gtatcaatag ttcattctgt attattacaa attagcattc |
| 121861 | aattacatga atacattgca atttattcat ttgtgaatgg acattgcatt atttccagtt |
| 121921 | ttggctgtga caaagattca tgtggaagtc tgtataggga catatgcttt catttctctt |
| 121981 | gggtaaatgt cttagagtag aatgcctaag tcatatggta gatgtatgtt taacttttaa |
| 122041 | agaaattgcc aaattgttgt ccaaagtgtg taccatttta cattcctggc agcagtgtat |
| 122101 | gagagttcta gttcttcacc atcccccttc caacacttgc tatagtcagt cttgcaattt |
| 122161 | tagccatttt aataaatgtg tagtgatatc tcactgttgt tttaatttgc atttccctag |
| 122221 | tgtctagtta tgttgtgtct ttacttttgt ttatttgcta tcattatctt ccttggtcaa |
| 122281 | atatctgttt aataattgat acatttttta ttggactgtt tgttttctta ttagtaactt |
| 122341 | tggagagtgc tatatatatt ctggatacaa atcctttaac aaatgtatga tttgcaaata |
| 122401 | ttttctccca gtctgtgatt gtcttttcat tctcctagca gtcttttgaa gagcagatgc |
| 122461 | tcttaatttt ataaagttca ctttatgttt ttttttattt tgtggattgg acatttggtg |
| 122521 | ttatatctaa aaaaaaatct gcctaaccca tagccttatg tgttttcttc tagaagtttt |
| 122581 | atagttttag gttttacatt tgggtctatg gtccattttg agttaatttt tgtatatggt |
| 122641 | ataagatgca gattgaagat ttttttttaa tatgtggaaa actgattgtt ccagcactat |
| 122701 | ttattgaaaa gactaatttc tccattaaat tgtctttgta cctttgttgt gaatcatttg |
| 122761 | cctatatatg tgtgagtcta tttctgaatt ctcttttctg tttcagtgat tcatctatgt |
| 122821 | tgatgccaat gctacactgt cctgataact gtagttaata aagtcttaaa gttaggtagt |
| 122881 | aaaagtctgc tattttcttc cttttcaagg ttgcattttc ctattccaag taatttgcat |
| 122941 | taatcaaaaa gtacatggaa aatgtgtatt ataacaaaag gtatggaatt ttaaaatttt |
| 123001 | caaaattctt ttgcatcaaa gtaaatgtgt actaacttgt tataatatgt gtgaacagga |
| 123061 | gctagtttga ggcactaaaa aggataaatc atcaatttaa aaacagcccc tatcagagca |
| 123121 | acatgaattc tgataaaatt gaagcaagaa caaatatcaa atttatcgtg aagcgtgggt |
| 123181 | gaaaaaaggg tgaaatcatt gatgctttat gaaaaattta tggggaaaat gtcacattat |
| 123241 | caaacagata actcatttta agaacagatg atatgatgtt gaagtcgaag cccacagcag |
| 123301 | cagaccatcc acattaattg ataggaaaaa aattcatctt gtttgtgtct taattgaaga |
| 123361 | gcactgatga ttaacagcac aagtaatagc cacaaccata tacacctcaa ctggctcagc |
| 123421 | ttacacaatt ctgactgaaa aattaaaatt ggccaaagtt tccactctat gggtgccaaa |
| 123481 | accattgtgc ccagatcagc tgcaggcaag agtagagctt ttgacggaaa ttttaaacaa |
| 123541 | gtgggatcaa ggtccagaaa gatttcttaa aagaacatta ataggagatg aaacatggat |
| 123601 | tatcagtatg accctgaagc caaagcacaa tcaaaacaat ggctaccaag aggtggaagt |
| 123661 | ggcccagtca aaataagagc agaccagtca agagcaaaga tcatggcaac agttttttgg |
| 123721 | atgttcaagg cacttttctt gactttctgg agggtcaaag aacaataata tctctcatta |
| 123781 | tgggagtgtt ttgagaaagt caaagcgtta gcagaaaaac gtctggcaaa gcctcaccac |
| 123841 | agagtccttc tccaccatga caatgttcct gttcattgtc acgaaacaag ctctcacgaa |
| 123901 | ataaagctct aattttgcaa gaatttcaat gggaaatcat taggcagcca ccttacagtc |
| 123961 | ttgatttggt ttcttctgac tttttgtttt ctaatcttaa aataatcttt aaggggcacc |
| 124021 | catttttcct tagttaataa tgtaaaaaag actgcattga cataattcaa ttcccagggt |
| 124081 | cttcatttct taaggaatgg actatttggc cggtatcatt gcttacaaaa gtgtcttaaa |
| 124141 | cttgatggag cttatattga gaaataaagt ttatattttt cattttaatc ttttaattat |
| 124201 | atttttccac aaaatttttg aagtcctctc acatatataa atttttggaa tcagcttatc |
| 124261 | aagtcatgta catttactgc tatatcacac tctgttttgt gaatattcca aaattatagt |
| 124321 | ctatgattta tattgttgtg actattgcat aatttacata tcaatgtact gatgatcata |
| 124381 | ttttgcaata tttttgttat tgttattcta ttatgaacag tgctactaat ataaatagta |
| 124441 | tttgccacct catacacagg tgcaagttta ttgtaatagt cttagtatta aattttctga |
| 124501 | gtcatagttt agcaaaagct caacttttca caggatattg ctggattgtt ctccaaagta |
| 124561 | gctaagttat actcctatta gccgtatatg agagattcta atcatcttca ttcttcaaca |
| 124621 | tttactattt tcagacttac taattttgcc agaaaaaata catgacaatt catacccgta |
| 124681 | cctgtagtgg tcttgatttc catttactca gaagacattg gacttttctt cttatgttta |
| 124741 | tgggctatat tttcccttct tttatgatat tactgttcat gtttttgctc atcttattgg |
| 124801 | gttatgggcc attttatttt atttttattt tttatttatt tatttttttt tgagatggag |
| 124861 | tctcgccccg tcctgcagac tggagtgcag tggcactatc tcgactcact gcaacctcca |
| 124921 | atgggccatt ttatatccta acatatgaag tatctgttca aatcttttac catttttttc |
| 124981 | ctgtaggtgt gcttgtcctt ttcttgttat aataaaacag ttctttattt ttgatactaa |
| 125041 | tcttttagaa agtgttctgt tgggcbgggc atggtggttc atgcctgtaa tcccactctg |
| 125101 | ggaggctgag gtgggtggat tacctgaggt caggagttcg agaccaacct ggccaacatg |
| 125161 | gtgaaactcc atctctactg aaaatacaaa aattagcccg gtgtggtggc gggtgcctgt |
| 125221 | aatcccagct actcgggagg ctgaggcagg aaaattgctt gaacccagga ggtggaggtt |
| 125281 | gcagtgagct gagagcatgc cactgcactc tagcctgggt gacagagtga gactccatct |
| 125341 | caaaaaaaaa aaaaaaaaag agagagaaaa gaaaatgctc tgtcagtggc aaatatctct |
| 125401 | tacttttttt ttagttttct ttgaaacctc ttttaatgaa tagaaatttt tttattttaa |
| 125461 | tgtagtttaa ttcatctatt tttctttttt aattgtcttc agtttcttta gtcttgtttt |
| 125521 | cataatcttc tggatatgag tcttgcaaaa catcttttgt taattttctc atggtttttg |
| 125581 | ttataatagt atttagagtt attttatata tttatggtta atataaatca aattgcatgt |
| 125641 | tgttcttgct ctaaattaca gattcaataa tttgcaacaa aaccttatct ctttgtaagt |
| 125701 | ccttaagtta attaagtact ctggatatct tcacatataa atacttgtat cagctgtctt |
| 125761 | ctctcttact actccaatta aaatccagta tctagaatag ggaaaaacca ggtgaacatc |
| 125821 | acccttacag agatttattt taagacataa tttagggtgt gctccatact gccttggcta |
| 125881 | tgtactagtc tatatattct agaagggtca tatatgtgca caaattgatt tgtgtagcca |
| 125941 | aaagctgctc cagtataaat gtgccaccag gatccatttc aaaacattgc ttcctcttgc |
| 126001 | caaggttagt gcagctatgc ttgctatcac tcaagtaacc agagaatgcc agcgtccgta |
| 126061 | ctctcaaaaa gacagccttt tctgcaccat tacaattggt gtcttttgtc cctttctttt |
| 126121 | cactgatgga aatctttttt atcattccaa tcaaagatca ctaccttcct aggagaagtt |
| 126181 | tcaatagaaa gaaaaaaatg ttatacttcc tcagaaaaat agcactttct tcatatagca |
| 126241 | agttttactt tcagaaaagt tgagaaaata aatactatgc ccccaggttt cccgttattg |
| 126301 | gagattgatt gctatgttga atgtgctata gctttgctgc ttaaacccaa acgccaagtg |
| 126361 | caggagtatc ttctttacta acgcatttaa tttaccaagc acacttagtg gctggtattt |
| 126421 | cttacctttc taagggtgca ctcacttaca ctgccctcaa tatatgtaat taggataaaa |
| 126481 | ccatagaatg gaaggcatta aaaatgaggc aggaaagcac aggcagagtg ggatgaagcc |
| 126541 | agctttgctc tgaataggac ttatatgatc agtttagttt tattccaaac atgaacccca |
| 126601 | aattcaaccc ctttgacctg agtactccgt cccttaaatg gcaaactaaa cattcgtgaa |
| 126661 | aatttaatgg tgaggaaatg agtaccttct taagtttcag atggtgaaat ggcaccaggg |
| 126721 | gagcactttc agtggcaggc agaagtgtgt ggatgtgcta gatcaaacct gaaatatttg |
| 126781 | ttccttcatc ctgcagtccc tctccaaact agccagccac tgagaccttc tgacaggaca |
| 126841 | cccccaggat gtcacccaaa aaaggtattt acaaaatcaa gactgtctgt aggacactat |
| 126901 | gtctttctga gaggtataaa ggtatttgaa aactttggaa agtttgtttt acaatttttg |
| 126961 | ttccctcttt tggggctttc cctttttcat ttttttctgt gggtagtttc cctttgtttc |
| 127021 | ctgagaaatt gagaagggtt taatatctct ccaccttctt ctcttttatg gatgaatgat |
| 127081 | ttcaccaact ggtaagtgct aaaactgacc tcgctttcac tcagaaagac aatggaggaa |
| 127141 | aggaaaatgc actgagattt cagcatagca actcaaatgc gggctttatt ccacagcagt |
| 127201 | gacctattgt cagattggtg tgtgagacag tttttgaaag gggatgtgtt ctgcttccgt |
| 127261 | ttatgagtta agcaaagttc taaattaagt tgagaagagg aagcaggtaa agtcagggat |
| 127321 | agcagagaaa ggagacatca aaccctattg ttgtaggata tttatgtgaa ggaatgagca |
| 127381 | acgtgataaa acacattttt gcttgcbgtg acttgccata gggtgcattt tcagtactgt |
| 127441 | gtggataatg tgtggcatgt tcattcatca gtcattctgt gacatttatg tagcttacac |
| 127501 | ttggatctat tttcaacaac atttcagttc cctaaagaca aactggcaga attttcaaat |
| 127561 | aggaaaaacc tggacacaca catttgcttt tctgtataga ctgtctagtc cagataatct |
| 127621 | tgctatcagt cattttgtta gcttactcat ttaatcttag aatggaaagt ggaaacagag |
| 127681 | acctcattta gagataatct agtaaagcca tccagcttca ggctcatatc ttgggaatct |
| 127741 | gagcagtacc ctatttaata tgctcatttt attaacttta aacacctgga gaacatcatt |
| 127801 | attgagaagg tcattgcttt tgaaaaaaca aaaacaaaac aaccacttct tatgaggctc |
| 127861 | tacacatctt tccaaagaag ctattggaaa tggcccaaac tttcatatta actcttaaca |
| 127921 | gggtggcgtg gtttttcatt agtaagaaca ggattcaaat cttatggaag atggtagatt |
| 127981 | tcaacacctt agcatttttc tcaaccctgg gatcttcaag ttcctcaggc tatttaggta |
| 128041 | gttatagtgc tggcaaactt tttaagtgcc cagtagtttt tacaaaaact tgtaataaat |
| 128101 | ctgctcaggc tatcttaatt gccaaccatc tcattaagca agtcaaatta gaatggatta |
| 128161 | ttcctgctgt ttattagtgc tgcatattat ttatcattta ttaaccttat tgtgctcaca |
| 128221 | tagcaggtat tttaaagttt aacagcttta ttttaattat gaatgtaata tatgctgttg |
| 128281 | cagaaaatat gtaaacttta gagaaaatgt actaaatgat gcactgatga aaatacagaa |
| 128341 | tcccaccaca aaaatattat tatcaacatt tgggtattct cttccaaact ttttatatga |
| 128401 | gagttatgtt tggtttatac ataataaaat gggtaaaaat caataaatag aaagcaaatg |
| 128461 | tttttaaggc agtacttggg cttcataaga atggaaaaaa atgatagact tgcctggtgt |
| 128521 | ggtttgcata atgtcacaga atcataacaa tagctccatt attgagtgtc tattatgtgc |
| 128581 | caggttctgt acaatgttca gtgatgcgtc actgtgatga ctagaggtaa aggaaagata |
| 128641 | agtgatgcct tactatgatg acttccaaca agctatgaag ggacagaaca agcctttttt |
| 128701 | tttaactttt attttaggct tgggagtacg tatgaaagtt tgttacatag gtaaacatgt |
| 128761 | gtcacagggg tttgttgtgc atattatttc acacccaggt acctatgtcc agaacccaat |
| 128821 | agtttagttt tcttttctcc tcccctccct cattctatcc tcccccctca catagacccc |
| 128881 | agggtctgtt gtttccttct ttgttttcat aagttcttat cattatcata cttataaatg |
| 128941 | agaacgtggt atttggttgt ctgtttctgc attagttggc taaggataat gtcttccagc |
| 129001 | tccatccacg ttcccacata agatataatc tcgttctttt tttatggctg catggtattt |
| 129061 | catggtgtat tttatatata tagatatata gatatatata taaatatata tagatatata |
| 129121 | tataaatata tataggtata tatatgtcac atatatatat cacatataca aggatatgtc |
| 129181 | ctctagctcc atccatgttc ccacataaga cataatctcg ttcttgtttt atggctgcgt |
| 129241 | ggtatttcat ggtgtatttt atatatatat atctatataa aaatatatag atatatatat |
| 129301 | cacatatata tagcacatgt atctatatca catatatata tatatcacat tttctttatc |
| 129361 | cagttgggac agaacaagtc aactggcaag tccattgctc agcttgttga aatactctgc |
| 129421 | acagaatgca aggaaaaact gttttcatga ggtagcccat gaagcgatga agcaaggcaa |
| 129481 | atttatctgc ctggataacc tcctgtatgc tgttttcact ccatggagag tcaacacaac |
| 129541 | agacttctga gttgtattat cgagctcctt caacagggca ttgctgtaag atcctagatc |
| 129601 | ccacactcca tggtgtgatg ttttagccaa gtctggaagt agcacaggcc ctaaaatctc |
| 129661 | atgattgcag gttgtcaact ctgctgtagg cttgccactt caagggcaag tggttggttg |
| 129721 | gggctgggat ttgggcagag ccatagaaag aaagggaaca ttcaagggaa tttgatgatg |
| 129781 | tgcatgatgt ctctgtctga tacaaaaaga aaaaaaaact ctaaaaacat ttgatacata |
| 129841 | gaaatactag tatttggtga gattgaatgt gggaagatga gggataagaa ggtattaaga |
| 129901 | atgactccca agtttctgac ttggtcagtt gagtagaagg gaaattttta tttttatttt |
| 129961 | tatttttaaa atttcagtaa gtttttaggg aacaggtggt gtttggttac atgaataagt |
| 130021 | tctttagtgg tgatttctga gattttggtg cacctatcac ctaagcaggg tacactatgc |
| 130081 | ccaatgtgta gtctcttatc cctcaccccc catcctttcc cttgactccc caaagtccat |
| 130141 | tgtaacattc ttatgccttt gtgtcctcat aatttagctc ccaattgtga gtgagaacat |
| 130201 | atgatgtttg gttttccact cccgagttgc ttcacttaca ataatagtct ccaattcatg |
| 130261 | ccaacatcta ttattttttg attttttgat catgaccatt gttgtaggag ggaggtggta |
| 130321 | tcccattatg gttttgattt gtatttccct gataattagt gatgctgaac atctttccat |
| 130381 | atacttgttg gccatttgta tatctgagta gaaggtaatt ctaataaata aaacaggaag |
| 130441 | gcagcagaaa gaggtttgtt tgggatatgg cttgattatt tgcaagatat ttgctgtagc |
| 130501 | ttaggagaaa ggtggatgtt ggtgatactg attttgtcat tatctgtgag ttaaagtttc |
| 130561 | taaaatcacg tggcttccta gggaaagcat atagaatgac aagggaagat ggctagatgg |
| 130621 | ctagtaatgg caccctgaac aacttcagca tttagaggtg ggtggaggag gataagccca |
| 130681 | tggaagaaac agaaaggatg ggccagcgag atgagaggag aagctaaggg agtagacagt |
| 130741 | taccagaggg aaggtgggat cagttaagaa ccttagtatg ttgcaaagaa ataacctgca |
| 130801 | atgcggacca acccggggaa cgtctgtaaa aatgatgaca attcttaatt gtaatttaaa |
| 130861 | atgtaattgt attgagagct ctctctgtct ttgtctcatc ataatttgaa gcagattaac |
| 130921 | tacatattag ctagaaaaag ccctttgaaa ttttccttca atgtatcaat agattctatg |
| 130981 | actttttgta agtagaaact gcctattggt aaaatagcac ttaattgaga taatgcttgt |
| 131041 | gaaagccatt tctaaacttt gaattgacat acaaaataaa ggtggcaatc atcaccatgt |
| 131101 | tgctatactg tctttggcca tataaaattt ttttcaacaa tacaaagata agccttgtac |
| 131161 | aaattcttca aaattctaaa tttgtggtac ttgaataatg ttttactata tataaagatg |
| 131221 | aatagatagc atttcctaca ataaataaat aatttatata tcaatgtgtt gggattataa |
| 131281 | attctcatag aaattcactt tattaattca ttcattcatc tgatcacagt tcattcagca |
| 131341 | aataatttgt tttgttcttt ttaagtgctc tgggaagaag gtattgtgac agagtccctt |
| 131401 | ctctcaaagc gtttatagat tcatgtgttt ggggaaagta cagaacaatt attgaaataa |
| 131461 | aaccagagtc aggcacattg ctatgccagc tataaagagc ctatgaaaat taggaacatt |
| 131521 | gacacatttg ttcctaacct ggtttgtgct ttcaggattc ctctgaagtg gcagcactcc |
| 131581 | ttgggtttga aatgttcgtc agtaatgact gatggacatc aacaggtgaa tagacacaca |
| 131641 | aaaaacctat gaaacttaca taccataggt tactattcaa caataaaaag aaaaactgtt |
| 131701 | gatacatcta acaatgtaga tgaatctcaa aagcattatg ctattccttt ggctgataaa |
| 131761 | atggtgacac tcctttattt ctctttcttt catcccactt ttaaaatcag ccttagtata |
| 131821 | ctttaggcac aatatactat atacatttaa aatgacaatt tgacaagttt tgacaaatgt |
| 131881 | atatacatta taaaaatcat gataaagatt tccgccgcct caaaatgttt cctcatgcct |
| 131941 | tcttggaata aatccacaca ttccttcctt ctctacaccc caccacccat atactctacc |
| 132001 | tctagaccca agcaattgct agtctgcttt ttgtcagcgt aaattagttt gcattttcta |
| 132061 | gaatttcata ttaatggaaa tgtacagtaa gtactctttt gtgcctgact tcttacattt |
| 132121 | agtatagtgc ttttaagatt tatccaaatt gttgggtgta tcaataattt gtttcttttt |
| 132181 | attgctgagt agtattctat tgtatacgtg gttcacagtt tatccattca ccagttaatg |
| 132241 | gacattccag ttgttttata tttggggctg ttatgaataa agctgatgtg aacatttgaa |
| 132301 | tagaagtctg ttgtgaatgt ttggattgga attgcbgggt tgtacagtaa gtgtttgatt |
| 132361 | aacttcataa gaaactgcca aatgattttc caaagtggct acatataatt ttgcattttc |
| 132421 | accagcagtg tatgagattt ccagttgatc ctcatcctca ttcaaaacct ggaattaaga |
| 132481 | gtgaatcttt taaattttag ccttttgaga aaatgtgtag tggtacttca ttgtagtttt |
| 132541 | agttcgcact tctctaatga ctaatgatgt ttaacatgct tttatatctt cttttgtctc |
| 132601 | catctattct ttgttcttca ttctcttttc ttcctttaga gtaagtattt tttattttat |
| 132661 | tccattttca ctattggtat attatctata aattctttgt tgttgttcca ttttagtggg |
| 132721 | tttacaatat aaatctttaa cttatcacag tctgtcatca aataatggta taccccttac |
| 132781 | atatgtttta ggaaccttac agtggtaaaa atatcagttc cccatcctgt acttgttgct |
| 132841 | attgttttca tgcattttat tctgtatata ttataaactc tacaatctat cattattact |
| 132901 | ttttgtttaa atattaaatt ctttaaaaga gatttttaaa aataagaaaa tagtctttca |
| 132961 | tattttctca tgtatttgcc atttttattg tttttttttt cttccatatt tgagatccaa |
| 133021 | atttctatct ggtatacttt tccttctgcc tgaataacta cctttagtat ttttttgtag |
| 133081 | tacagatctg ttggcaatga ttatctgaac ttctgtatgt ctgaaaaagc ctttagctaa |
| 133141 | tttttgaaag ttttttgatg gacatatgat atggtttggc cctgtgtctc catctaaatc |
| 133201 | ttatcttgaa ttgtaatccc catgatcccc atgtgtcaag ggtgggacca ggtggaggta |
| 133261 | attggatcat gagggccgtt cgccccatgc tgttctccta ataatgagtg agtctcatga |
| 133321 | gatccgatgg ttttataagt gtctgacatt tcccctgctt gcacttctcc tttctgccac |
| 133381 | cttgtgaagc aggtgcctgt ttccccttca ccttctgcca tgattgtaag tttcatgagg |
| 133441 | tctccccagc cttgctgaac tgttagtcaa ttaaacctct ttcctttata aattacccag |
| 133501 | tctcaggtat ttcttcatag cagcatgaga atggactaac acggtacata caattacagg |
| 133561 | ttgctagtcc cttttttccc ttcagtgctt taaggatttc actcttccat agtttctgat |
| 133621 | gagtagtctg accattccta actttgtgcc attattttct catttttttc tgactctccc |
| 133681 | ttctgggact ccaattacaa atatcccaga cttcctaact ttggcaggtc aagagatcga |
| 133741 | gaccatcctg gtcaacatgg tgaaacccag tctctactaa atatacaaaa aattagccag |
| 133801 | gcatggtggt gggcgcctgt agtcccagct acttgtaagg ctgaggcagg agaatggctt |
| 133861 | gaacccggga gggggagcct gcagtgagct gagatcgcgc cactgcactc cagcccgggc |
| 133921 | gacagaccga gaatccgtat caaaaaaaaa aaatcattca aaagattact gatgcttgtt |
| 133981 | tcattttttt ttaatttttc aactggtaat taatttatta aaatagttga cttatacgtc |
| 134041 | tacaatggtg acttccatct caactcctgg ctcaatactg atggaagtaa tctgcttaac |
| 134101 | aatctcagaa ggactgagca agtcaatgag tcgcttgtgg attctcatct ggaaatgatc |
| 134161 | ccatggctta gaaccttcac cacatggagt tttttcttgt agtgattctc aaagtcttag |
| 134221 | taggcatttg aactggtctt ttcactttga gattcttttc ctttgctcct ctgatcaagt |
| 134281 | cagcacacac cttcgccagg gattttacat tgcagcttgt tagagtgatt cgaattcggt |
| 134341 | gaattgccat cttcggctcc gtgggtgttt ttccggtatc cttaaaagcc atcactgctg |
| 134401 | cgcggcttcc tgacctactt gttccggcaa gagtgaacag tggtgagtca ggagcaggtg |
| 134461 | cgggcggatc agagctccac aacacctacg accacctctt cttcaaagag gtgtttcatg |
| 134521 | ttttaaaatc ttaatttcct tctgtttcat tttggataaa atctgtcttc aagtttggtg |
| 134581 | cctttttttt tgggccaata tctaatctaa agttcatcct atccagtatg tttctcattt |
| 134641 | caaatattgt attttctatc tgcagagttt gatttgtatt tgaaaacgtt ttttaatctt |
| 134701 | tctcattgtc ttaaacattt ggagtatact tataataatt gttttactgt tgttatctgt |
| 134761 | tacttctatc atctatgaaa ttactatatc tgtttctatt gattgatttg attactattc |
| 134821 | tggttgggag gcaatatttt ccctacttct ttgcttactt ggtaattttt ttatcaatga |
| 134881 | aaatttataa aagtgaactt catttacaat tggatataat gaattttaca tttttgtgtc |
| 134941 | ctggattttt ctgtattcct ttaaatattg ttggatttct tttttttccc ccctccggga |
| 135001 | tgcagataag tttcttggaa ttagctggat gcttttgaga tttgctttta agatttgtta |
| 135061 | gggtgaattc agagcagggc tactttgatc taggaaatag ccctctgaag ggtctcatgt |
| 135121 | tctgtgagat aggaggtctt tctcctctga ctggttggta gcctaagcta ttcctgattc |
| 135181 | ttgtaaggtc tggacactgt tctgcatact cttttctgat agttctttcc ctggccttag |
| 135241 | tagttaccac acatctatgc acatagcggt attcagccaa agtttgaaag gattcaaaga |
| 135301 | tctccagagc tctctctgta tcactgtttc tgttattcta tcccacaaat tctaagtgtg |
| 135361 | tgaaactccc tgaaatcaga actatgtctc ctcaactcag tgagaccaca agccactgtt |
| 135421 | tgggtttccc ctgcccatgt tgcagcctag aaactctcct caggcaaaaa gatcaggcaa |
| 135481 | catcggggtc atctcatttc cttccctttc agagatcact gtaattgcct attgaacaat |
| 135541 | gcctgaagaa agatgtttca tatattttgt ctggctttct agttgcttaa ggtaagaagg |
| 135601 | taaatgcatt cctagttact gcattgtggc tgggagcaga attccaatac tgtttttttt |
| 135661 | ataatatgct tttacttaat tatttgagga ttccctttag ttatcataat tttacatgat |
| 135721 | gtaaacaggt aaatgagaat atcttcaaaa tatatttttc agttttgggg tggttctctc |
| 135781 | agttctggtt tttgccaaat ataatagatt tttagtatct gagttccata tgacaaaatt |
| 135841 | tataaatctt gggtgtatcc atatggtatg ttataatcgg aaaagataga tgaaattccg |
| 135901 | cttttgtcta atgtctttat aggtcttgga gaaacttgaa gaagggttgt aaagtccata |
| 135961 | actttacaat gagacaaaat taaaagaatc caagccaaaa ttgtcctaga caatgaaaag |
| 136021 | tatcgctatg cagaagccaa atctcttaag ctcctccacc atatcagtca tgacacagtc |
| 136081 | tatagtttaa caattgtttc aatttggaaa ttaattttta gtactaattt aaatatttca |
| 136141 | tattagtata tgagaattct tccttctgtt caggctgtgg tatggggaaa agtcagttcc |
| 136201 | catcaatgca gattcatatc taccagcaca gtgattaaat tatcctcata tcctcttgtg |
| 136261 | tttgaacaag ctgtgtactt ttaattttaa tggatttaac tcaatattca atatatgttg |
| 136321 | attactgaat acaacaaaaa gtgtaataga cactccttga gaagctcata atctatttgg |
| 136381 | gcagatggaa caaatactta gaaaaattga aaggtagttt aggaccacag taaaaaaaaa |
| 136441 | ttgggggtgc aggatataaa gtatcaattg aattgtagaa gcaatatata ctgtgtattt |
| 136501 | tttcacctgg gtgagagggt gagactccgt ctaaaaaaaa aaaaaaaact atggttgttc |
| 136561 | accataacta aacatttaat ctctttcagt ctatcttact catctacaaa atggagataa |
| 136621 | tacctgtttt gcagtgcagg ccctgacata cgggtagagt tccataaatc ctagcttcta |
| 136681 | ttccttaact cttttaactc ttttcttcct tccttctttc tagctaattt ttaactctct |
| 136741 | atcctagaca acttttgcta aacgtgtata acatgtagta cctacgcata ggaatatgtt |
| 136801 | ttatagaaca gaattattga aaacagcttc ttacatttct gttatttttt caaaagcaag |
| 136861 | aattcatgta ttccttctga gccaagtaca tgcaataaaa agtaagagag attcagaaat |
| 136921 | tctgatgatt attctcccaa gaagagtgct tgctagagtg agggtcttgc agacacagaa |
| 136981 | ggagctgatg gggtgaactg ctggggtgtt acaaaaggcc ttcgggaaaa tacgtaatct |
| 137041 | aagtcaataa atcatagagt gaagattccc tcagggaaaa agggagtggt tctgaagaac |
| 137101 | ttacaaattt ttccaggcaa tagccacaga tgctagcaca agggcattat ggccacatag |
| 137161 | tgtgagcaaa agtgcttaac tttttgccct tcttctaata tttactctat tagcccagac |
| 137221 | aacatgagat ccagaagcct catgatgagt ggagaggaaa tataaatcag aagaaatggt |
| 137281 | caaaaagatg cctatattca cttccgcact taggtctcca agcctcagtt cctggcatga |
| 137341 | gtaggtagag gagaggagct tggaatagga tgtgagatta cagttttaat atagactgga |
| 137401 | ctggatattt gaatacctga aaatgaatct gaattctcaa aatgaggtgg ttcctgcaac |
| 137461 | tgaaaataac aagaaagcta caggatcttc tgccatgtta tccaggggtg gaaaagtgaa |
| 137521 | attcagtgga gccaggttaa agatggagtt tgggccggga gtggtggttc atgcctataa |
| 137581 | tcccagaact ttgagaggcc aaggtgggca gattacttga ggccaggata tcaagaccag |
| 137641 | cctggccaac atggtgaaac cccatctcta ctaaaaataa aaaattagcc aggtgtggtg |
| 137701 | gcatgtgcct gtagtcccag ctactggggt gggtgaggca tgagaatcac ttgaacctgg |
| 137761 | aagggggagc ctgtggtgag tcaagattgc accactgcac tcccgcctgg gcaacagagc |
| 137821 | aagaccctgt gtgaccttgt gtccaaaaac agacaaacaa acaaatacca tgtggagttt |
| 137881 | gtttactctt taaacaaata tccttcatgt gttgaactga actataatta tagttagtag |
| 137941 | ggcacatagc aggttttaat aagatatcta gttcttcctc ttaaggtaac tcgttagaac |
| 138001 | ttttatgaga aagaactaaa gaatttctaa tggggcagct aatccactct tttgtgcact |
| 138061 | ctgttctgct gatgatctga tgttagtcgg ggtttctgtt tgagattcct gagccttaac |
| 138121 | tgctgatgac agagttcaaa atcattgctt tctaactttg agaatgactg cctaatatga |
| 138181 | catcattttc tacccctgag tgctaatagt ctaccttatt cctcctgcct gtccttctcc |
| 138241 | ttgaggagga ggatcatcaa tgcactgtgt ggattgaagt ggcttcatac ccttttgatg |
| 138301 | ttaatgttct cctttgctta aagtgtgaac tgaatatgga tccctcattt atgaagcacc |
| 138361 | atgacacttc ctgggtaatg aacaattaaa cagacttctc agcaatagtg actatttttc |
| 138421 | tgatttttca cttcatggac aatattgtat gcaagttaaa tttgcaacta gcttgaggct |
| 138481 | tcacacatgg tgagtttgag aaattgaatt ctctacctca aatgtatttc cttttccttg |
| 138541 | aactctacat tcaggtcagc tgtccagttc ttaaacaaca ggttttggag aggcaatgag |
| 138601 | taataaaata gcattttctc cttcaattct atctgcattt gaaaaaaata accttttatt |
| 138661 | tttgattcaa agaagagagg agttttcttg tctttctaca tctgttccaa ctcctccaaa |
| 138721 | tgcacaaaaa ttttttcaga gcattccaga gtttctcaga ctgcacatag aagattttgc |
| 138781 | atatagatac atcttggcca taataacctt ctactgaatt agtagaaaat agatgtattc |
| 138841 | taggttttca tatctttatg gcaatatgaa tagacaaggc tcttcttgca aaaatatctc |
| 138901 | ctgaactgtg cgggagatgc ttggaagacc cagtcaaaat tacgaagtat cccaagtttg |
| 138961 | tatcctcctg tggcttcccc gtattacaat aatgtcattc tgatccaggt tccagtactc |
| 139021 | tcaagccctt gtccttccct tttcatttta gcttgcattc atccaagtat ttggatggaa |
| 139081 | attatcaata ccttatacga gaacgtatgg gaggctgagg cgggcggatc acgaggtcag |
| 139141 | gagatcgaga ccatcctggc taacacagtt aaaccctgtc tctaccagaa aaatacaaaa |
| 139201 | aattagccat gcatggtggc acgcgcctgt agtcccagct acttgggagg ctgaggcagg |
| 139261 | agaatcgctt gaacctggga ggcagaggtt gcagtgagcc aagatcacgc caccgcactc |
| 139321 | cagcctgggt gacagagcga gactcatctc aaaaacaaac aaacaaaaag aatgtaaatg |
| 139381 | attgggagtt aaaattatgc ttttcagttt tcagccatgg atcatgtttt cttgccttgc |
| 139441 | ctgattccta gagtctcact ccatgatgtt tcctctttat tataaagatt agatggcttt |
| 139501 | gcattgctgc catgtggcat ttatacttct caagcagctg tgtcatctta ttctaccgtt |
| 139561 | taaacttagt tttactttaa gtccacatct aaactgtttg cctgcatgcc cagcactttt |
| 139621 | tccaccctct acattttatt tgactggagc aatcacctta gcctctctag gtctttgctt |
| 139681 | atcatcactc tgctcattct tcaagcctca cttgattact gcctcctctg tgttgctttc |
| 139741 | ctggatttcc tcagtaagac atagtttctc cctttcatac ttctgtggta ttaagtatgc |
| 139801 | accagttaca tgtgactcat ctccaagtgc tttggcatgg agatattatt tatgttcctt |
| 139861 | atatgactac tgaccacttt tttctagact aaatattgaa acacttggcc agataatttt |
| 139921 | gcttaatttt gagtttcctt ttatactttt taaaacaaat ttttgaaatt aaattacatt |
| 139981 | ttgttaggtt gcaaatctga aaaagtccaa ggatgaggtg agtttgttgt ggtcagttgg |
| 140041 | gtagttgcta aactcatagc tggtttgtgt ctgctgacca gcacattgtt agcctagaga |
| 140101 | aagtctacat ggggaaccag attatgatgc aagcgttgct tggagagccc ttgatcaaga |
| 140161 | gcaaaacagg ttggtagact cgagggatag ttcagctagg tctgatggaa cattccagtt |
| 140221 | ttgccaacag aaggaaaaac attggaaggg cttatcttcc ccaggatctg gatctgttcc |
| 140281 | actgggtaac atttaataag cactaacatg gtctcttctt tcacttgcca tttcctgtgc |
| 140341 | ttctttttcc aactactaca tctcctttga cacattttct ttttcttttt cttttttttt |
| 140401 | ttttgagata gagtcttgct ctgtcaccca ggctggagta cagtggtacg atcttagctc |
| 140461 | accgcaacct ccacctcccc ggtccaagcg attctcctgc ctcagcctcc tgaattgctg |
| 140521 | ggatcacaga tgtgcgccac cacactcagc aatttttttt ttttttttgt atttttagta |
| 140581 | gaggcagggt ttcaccatgt tggccaggct ggtctcgaac tcctgacctc aggtaatcca |
| 140641 | ctggcctcgg cgtcctagag tgctggcatt acaggtgtga gccactgtgc ctggcctctt |
| 140701 | tttaagtgta tcaataccat cattttggct gcttattctg ccctgggagc cattctgtct |
| 140761 | ctttaggaag tttttgcgtg tatcagatct catcttttat attttgatgt ttaatagcat |
| 140821 | ttaaaggatt ttgttttgtg ttttttaaat gatttgaaaa caagtgtgct cactaattaa |
| 140881 | taaacagcag attcaaactt ttgttgcttt ttcaagaaaa aaatgaaaaa ttctgtaaca |
| 140941 | atatatctta gccataaaac acttgtaatc agcactcctc cagaaatttc aggatgtaag |
| 141001 | ttatcttaat gattatacta gaaaatcttg gaaagcaggg atatggctaa ctttttataa |
| 141061 | catgtatccc tactgtcaca acaagagtta agacttaata aatacagtca tgtgttgctt |
| 141121 | aacaactggg atatattctt gtaaccaccc aaggggttca ttttgcctgc tacctggata |
| 141181 | cagccaattt atcaagaacc aggaattgca gtagagaaag ggtttaattc atacagagcc |
| 141241 | agctgaatgg gacactagag ttctattact gaagtcagtc tccctgaaaa ttcagagggt |
| 141301 | agggtttttc aaggatagtt tgaagggcca gggaatggtg ctgctgattg gttggagata |
| 141361 | caatcatagg gatgtggaaa atggtcctca tgcatttctc aagtccactc cttttcttag |
| 141421 | ttcctgctta gcggaggtgt tagaatttgc ttcttggtgg ggccacagga ctggttggtg |
| 141481 | ggtccaggtg gggccaccag ttgtcagaaa tgcaaaaacc tgaaaagaca tctcaaaaga |
| 141541 | ccaatcttag gttctgtaat agtgatgtta tttgcaggag taattgggga agttgcaaat |
| 141601 | cttgtggcta atttgttagt cctacaaagg cagcctagtc cccaggaaag aagggggttt |
| 141661 | atttaaggaa agggcbgtta tcatctttgt ttcaaagtta aactataaac taagttcctc |
| 141721 | ccaaagttag ttggacctgt ccccaggaat ggacaagggc agcttggagg ttagaaacaa |
| 141781 | gatggaatct gttagtttag atctctttca ctgtcataat tttgtcactg ttaacgtttg |
| 141841 | caaaggtggt ttcactgtag actttataaa caccatatac ttagactata ctaagtttat |
| 141901 | taaaaatatt tttctttctt tagtaacaaa ttaaccttag cttactataa gttttttact |
| 141961 | ttttgaagtt tttaattttt ttgaactttt gactcttgta ataacagcat aaaacacact |
| 142021 | ttgtacaact gtacaaacat attttcttta tatacttatt ctataagtgc tttctatttt |
| 142081 | cattttttcc ctttaaactt tttaatttaa atgaagacag aaacacaata ccctaggtct |
| 142141 | acacaggttc tggatcatca atatcattat ccaccacctc cacatcttgt cccactggaa |
| 142201 | gatcttcatg gacaataaca cgaagctctc gtctcctgtg ttaacaatgc cttcttctgg |
| 142261 | aatacctcct gaaagacctg cctgaggctg ttttatagtt aatttccttt tttacaagta |
| 142321 | gaaggagtac actctaaaat aacgattaag agtattgtac agtaaataaa tacagcagta |
| 142381 | acagagtcgg ttattatcat tatcaagtat tatgtactat acctaattgt gctacatttt |
| 142441 | tgtatgcctg gcagtgcagt aggtgtgttt caccaggatc accacaaaca tatgagtaat |
| 142501 | gtgttgatct atgacattac cagagctacc atgttgatat aaacaggaga cagggaaata |
| 142561 | ctgggtagaa gagggcagtt ccctggcaaa agccttactc ccaagcctgg aaacctgcag |
| 142621 | tcctaaatgg gaataggcat tcctgtttgt gcatccaaat gttgcctttt ggcctgccat |
| 142681 | gcccctctgt cctgtatcca tataaacccc aaatcccagg cttgatgagc agatgagcag |
| 142741 | acaaacagaa gagcagagga gcagaagagt ggtgctgcag agaaagagag aagagaagga |
| 142801 | gcatctgaat gtcaagagga gtttggatgg ggatggtcag agaggagatc agctgcagga |
| 142861 | cagccaaact ccaggggaag atcatctctc actccatccc ctttccagct ccccacccat |
| 142921 | cctgctgaga gccacctcca tcacccagta aaatcctcac attcaccatc cttcaagttc |
| 142981 | atgtgtgact tgattcttcc tggacactgg acaagaacct gggtaccaag agggcactga |
| 143041 | gctggttaac acttaagcca tctgcggatg gcggagctaa aagagcactg tagcacaccc |
| 143101 | actggggctt tgagagtcac aggcacacac ccctagatgc caccgtggga tgggaaccca |
| 143161 | aaagtgctcg ccctggctcc tgcacctatc tgtctacatg ctccctctcc tataacaggc |
| 143221 | ttgcacagca gtgactgcgc aacagatgag ccacacccct tccacatgtc tgcaaggggt |
| 143281 | atcagggaac tctccccttt caatgtcagt aggtaatagg aattttttag ctcctttata |
| 143341 | atcttatgat accacctttg tgtatgtggt ctgtcattga ctgaagtgtc gttatgtggt |
| 143401 | acgtgactgt atttattgag tggatggtca attgtatgag gtacttgaat aatgagtggt |
| 143461 | gtgaaccttg gttttatgac atcttctgag agctgctttc aggtgggtat aagaagagat |
| 143521 | atttctagtc taggtcccca ctccttttct tagttcctgc ttagtggagg tgttagaaaa |
| 143581 | aaaggttgga ctctggcact attttcctcc attggttata tggactcaaa gccataactt |
| 143641 | aaggaagaat gtctattttt agatttggtg actgatttag ataggttgct acagaaagtg |
| 143701 | gtacaaagat atttcacaca tagaatatca aatccagggt gctttagcac tgcattccaa |
| 143761 | tggcctggga tggatgggtg gaagccctgg caagctgaaa taaaccaggg caagctcctc |
| 143821 | tttggaattc acaagtctga caactggtga ctcaggctct agtcaggaga cctgaatgtt |
| 143881 | acgtctggca tcacgatacc ttagtttaca gagattaatc taacaggcct tcagttagtt |
| 143941 | tgctttattt taaaattctg cttagctttt tgttgcctac taaatttctc ctgatgtata |
| 144001 | tttagatgta ggtgaaggct gtagatgtat gcactaccaa caccagcttc tgggagggaa |
| 144061 | cattttccca ctcacaagca agtgaacttt gctttctcca tgaagaagaa tgacctgcat |
| 144121 | gatggctttc agaagtggag aacatggctc acagttccct ccacagttcc tccatggcct |
| 144181 | ttggttgcat cttctctgaa tggaggagct tgggttatgt gaaaagcaga gctaccagac |
| 144241 | tacataaatg ttttagtgag aaggcagcaa attaaaatat cagaaatttt gaaaggaaag |
| 144301 | gtttataata gttagattta aaacatactt tgaggccagg cagcgtggct tacgccagta |
| 144361 | atcccagcac tttgggaggc caaggcggga ggattgcttg agggcaggtg ttcgagacta |
| 144421 | gcctgcacta cataacaaga ccttgtcctg tctctattaa aaaaaaaaaa aggaaggaaa |
| 144481 | gggaagaaaa agtgctttaa tattcagagg catgagagat gcaatgagta atataggacc |
| 144541 | ttctctaagg acactggggg agcactctac agaggtggtt ctaatctagc ctggcaaatc |
| 144601 | tctgttccct tcatgccttg tacttccttc ttctttgtga ctttgctaat gttattcctt |
| 144661 | cctcttggac aaatttccta tactaaccaa aaggccccaa gacacagggg gaaatgtggt |
| 144721 | aaatctgaga ctggaaggaa attaccatct aatgtcactc ttgtacagta taactattgt |
| 144781 | taatattatt gcbggggtca ctcatttatt catatcacaa acactgactg agtgcctatt |
| 144841 | atttgccaag catgggcaat aaagagttga agaccaacaa agcccctgcc ttcccagaag |
| 144901 | cttatttgct tgtggggaaa ataaacagtg gatcaattaa caaataagta gataacataa |
| 144961 | tgtcacggcc aggcgtggtg gcctcatgct ggtaatccca gcactttgga agactgaggt |
| 145021 | gggaggatca cttaggccca ggagtttgag accagcctgg gcagcatagg gaaatcccat |
| 145081 | ctctaccaaa acaaaacaaa acaaaacaaa acaaaacaaa acaaacaata aacgaaacac |
| 145141 | atggtggcgt gtgcctgtag tcccagctac tcaggaggct gaggtgggag gatcacttga |
| 145201 | gcccaggaca cagaggttgc agtgagccaa catctcccca ctgcactcca gcccgggtga |
| 145261 | cagggtgaga ccctgtctca aaacaaacaa acaaacaaaa caaccaaaaa ccaaaaaaag |
| 145321 | taatgtcaga tggtgactgt tttgaggaaa ataaagcagg gtaggggaac tagtgataac |
| 145381 | tggtagattt gggtttctcc atctctggct gcttttgcat cctcagtaag gtgagagtgg |
| 145441 | caaaggaaat tccaggttga ctttcgggtt cagtgaattt tctaccccgt gtctcttcac |
| 145501 | tgccctttcc ctccccagaa cctctaaaat ctgatcctta gattctgcaa tgtaagtcag |
| 145561 | cttccttctt cctggttgcc agctcactac attcttattc ctggataaac caaaaatctc |
| 145621 | ttccttgttt tcatattttc aaaaagatgt agaaatctct cacctgctca tgttccttcc |
| 145681 | tttttgtctt gttggtatat atacctttta caattctttc actaccaaat gtagttggat |
| 145741 | attgggatgt agtcacaaca aacacataaa tacaattcac catttttaaa tggacttatt |
| 145801 | catttagtct gcttaaaatt gagataataa cacacgcacc agttgggttt ctctgagaac |
| 145861 | tctgggcatt tttaatcctg gcaattttta aattgcttag cagtaaaaca gtcagacaac |
| 145921 | agcacctatt gttataaatc cctgcctctt cttacctcaa tgaggctatc ctttctctct |
| 145981 | atcaattctg tcagcagaat taagaagagc cattgatttc cagtttgttc agcgtttttc |
| 146041 | ttcttaggat gggagtgatg acttctaacc tctttacatc tagagactca atttaagtct |
| 146101 | acttctttca ttcagccttc tctcttcatt ttcagctgcc tggaatgtct tctttctctc |
| 146161 | agttcctatc tttgacatta aaaatcacag tacttagtta tattctacct cacaccaatc |
| 146221 | attttacttg ttaatatctt ttttcctagt tagattgtga cattttggaa taaaagaaaa |
| 146281 | atatcctttt ttactttata gctctccaga tttcattaga ttagtataga ttcgagtgcc |
| 146341 | ttttatatat caggcaatgc ctagtgtata atatttttac ttatgggttt tagcatttaa |
| 146401 | cttatttaat gatcacaaca ttcctatgat gttatttgtt ttcataattt gttgaaatga |
| 146461 | aagctgatgg tcagaaaggt taaaaagttg tctggtcata atttttgagg aggcagagct |
| 146521 | aggttttgaa cgcaggtctg tctgattctg agaataatcc tgtgctcctt cagctgaacc |
| 146581 | acacagcccc tcttagtgct ttgtctccat agacactaag tgtattcttg ttgattgagt |
| 146641 | gaattaacga gtgaattatc tggagctctt tctgcttctg ttttcttctc tgtacaatga |
| 146701 | ggatcatcac atgaacctga tctaagaata gaatagtcca actgagttcc cttaatttgg |
| 146761 | taaccaagag agagtagagt taattaacta gaaccctttg ccatcacttc tctctaaact |
| 146821 | tttcatcatt ttcctctaaa agtccggtcc tgagctctat tacaagatag aacattttag |
| 146881 | cctctatcga agagagcact attaatattt tatggcattg gctccttttt tcctaatact |
| 146941 | gtgggattaa aataatgttt actaaattct ggatgttcga ttcctgtttt ttcatgatat |
| 147001 | tttataatcc aggccttctt catagagaca cagggctatg ttttataact gccagggtca |
| 147061 | agcaaagaac agcttgtgtg agatacttaa agaaatagct aagcagtaac ttcagacctt |
| 147121 | ctgtatattt tcctcaaaat tttctctcag tttttgtctc cctacaactc atttttagaa |
| 147181 | aacaaaaaca taacacaatt ttgccctttt aaaagataat acagcbgggt gcagtggctc |
| 147241 | acgcttgtaa tcccagcact ttgggaggct gaggtggggc agatcacctg aggtcaggag |
| 147301 | ttcaagacca gcctggccaa tatggtgaaa ccctgtctct actaaaaata caaaaattag |
| 147361 | ctgggtgagg tggcggcagg cgcctgtaat cccagatacc cgggaagctg aggcaggagg |
| 147421 | atcgcttgaa cctgggaggc ggaggtagca gtgagctgga attgcaccac tgcactccag |
| 147481 | cctgggcgaa agagtaagac tctatctaaa gaaaaaaaaa tgtactataa taatttacaa |
| 147541 | agtacagaaa atatctaaag gaaaagaaat aacatctcac aacctcatca tctagatatg |
| 147601 | actgcaaaaa ttttggcata tttcatttca gcttttaatt aatttatttt tgcagaagac |
| 147661 | atttatttca gatattctaa ctttataatc tgtagaattt taatgatatt tttgtatgac |
| 147721 | atctatcata aacatttctg catgtcattg aaatatttat aaatgttatt tttatttaaa |
| 147781 | caatgcataa ttgatataac ccttctctta aaattggtga tttcacatac ctggggatga |
| 147841 | gccgatgtga aacaaaagag aaagggtaag agatagctag tctcttgact catttttttt |
| 147901 | tttccatatg acttgaaaaa aagaacttat cttagagacc tctagaatag ttagcacaaa |
| 147961 | tagatttctc aatgtggtta agatgaaata cagatactga atttactctt ttatgtgaaa |
| 148021 | taaataaaaa ctggataaga tatatatagc aacagttttc aagacattat acctcagaca |
| 148081 | acaaaggata atgacctctg agagagggaa aacaaattag gtgagcctgt gattgcccca |
| 148141 | gtttactgcc tggagagaga ttccaggcca gggtgcagag agaagggttg cgaatggaaa |
| 148201 | ctattgctct ttctgagtta aggagagaga cctgaaagtc tgcaggtaca aagatagctg |
| 148261 | caattcatga gaaagagttc cagagaaaag aaaactgaac agagagtgag ctctggattt |
| 148321 | ctgtagtgtt cctgctgact cttctgctga atcatgatca gtgcatgtgt gtggctggag |
| 148381 | aaagaacctc tgaaaaggtt tagagggaca aatccttaga atcacatagg cctgggaata |
| 148441 | gctcctcttt ccaccagaaa gagtataaaa accatgaaat tcatgggaca ttaggtagag |
| 148501 | tacttagaag ggttttgctt cagcagtgag aaaaaattag tcctagacca aatgttgctc |
| 148561 | tgatcctaac aaaacttaaa aaatgagacc ctaaaggata aaactgtttc caggtaactt |
| 148621 | aaatatatac cagaacaaag ccgaagacta taggaatatg aaaatatcta gcagtcagta |
| 148681 | aggtaaaatt cataatgtct aatatgcaac caaatattac catgaatata aagaagcatg |
| 148741 | aaaataaata cccataattc caagaaaaat caattataca taattgcccc atcaatgaca |
| 148801 | cagatgatgt aattagtaaa caaagagcta attataactg ccaactctag ccattacaca |
| 148861 | tgttcaagtg gctagaggaa agatcaaatc acgacacaga agaaataaaa agacaataaa |
| 148921 | atatgtacag atgaaaatta caatatctga gatgaagcat aagcgagatg ggattcgcag |
| 148981 | cagattagat atagaagaag gattaatgaa catcaaaaca tgtatgacaa cagtagtata |
| 149041 | aagtctgtaa gaaaagaaat ggaaacatac tggtataagg ttttacacta tgtatgaaat |
| 149101 | ggtgtagtat cacttgaaga gagactgtga ctaaagacca ctgtaacctc ttagggaaag |
| 149161 | aaaaatatct ttttctaccc atcctgggtt cacagctgag gccactataa caaaaaacag |
| 149221 | attaataaca cagaagcata cacatttact taatataagt gttatgtgac acagtagcca |
| 149281 | tcataaggaa agaagactca aagaaacaga tctgagtatt tttatagtag gtttgatgaa |
| 149341 | gaatggatag tcatggagaa atataatggg gcatataata caatggggtt atgatctaat |
| 149401 | ggtagtaaac tgggggaaat ttagcbgggt ttgcttgttc agatttttct ccttgacctt |
| 149461 | ttgtcgtgag agacaaggat gtttcttttc tctgggtata gggagggcac ctctcacatg |
| 149521 | agggacttat gatctgcttc agagaagtgc cagggaagaa cagagtgacc gtcctgcttc |
| 149581 | tgcagttttc tcaaattcat acagcttaaa atatttaaca tgctaaggtg ttacattttg |
| 149641 | gggtagtata tcttgatacc catcacctga aaccaaccac tttataatag taacacaaag |
| 149701 | agttgtacat aatataccat aaaggtggta aaatggaatc attggaaaag tcaattcatc |
| 149761 | caaaagaagg ctgaaagagg aaagcaaaaa caaagaagag atgggagaaa taaaaattaa |
| 149821 | atggtaggga catgagcatc cattaatttt ggtatacagg aggggcttga ggggtgtcct |
| 149881 | ggaatcaatc cccctcagat accaaaggat gactgtatat tttttaaatt aagagacttt |
| 149941 | cattctttaa atattcttga cttacagttt tttaaaaaga aaaaaaatca aagcatagta |
| 150001 | tagttgttaa atcattttaa tagtatattc agtggctgaa atgacaggga gtatcaatca |
| 150061 | ttatttattc aatataaagt tatccttact gaaaaaataa gatataactg ggaaatatgc |
| 150121 | ttggatccct cacacatcca cttaatttct taaaggaaga aagatgaaac acatgtaaga |
| 150181 | acagaaacaa ataaaccatt tactgtaaca ttccaggaat tgtgggatcc ttttcttccc |
| 150241 | aagtttattt atttgcttat ctttatctcc tagtggtagt ttgtttcctt tggtagaata |
| 150301 | tatggcagtc ccacattggt gacacctggt gctcccttca tccaactgga tcactttgag |
| 150361 | attcagtttt acaccttttt agcattttct ttgtgttcat atatcagtta tgatgagcca |
| 150421 | ctgataatat tttgttctaa aacgaaacat attacttagt acttttttta ttgtctcgct |
| 150481 | gacttttaaa atttgagtga tgtacagact ttgttttttt aatgaaatat aaatattttc |
| 150541 | tcatggtttc taaattcctt ataaaattaa attaattcaa tctgcaaaaa tattaaatgg |
| 150601 | taagagaata aactcatcac ttagatgttc atattaaatg taaatggcct aaatacccca |
| 150661 | attaaaagtt aaaaatggtc taattgaata aaatacccat atccaactat atcttgccta |
| 150721 | taagaaaata ttgccacttg cagtataaaa atacaaatag gtaaaagata aaaggatgga |
| 150781 | aaaagacata ctattttaaa ctaataaaat attaattaat taattagtca gatttcagga |
| 150841 | cctttttctg aataattgat agaataaaca gacaaaaata tcagtaagga tacagaaaac |
| 150901 | ctccaaaata ttatcaatca ccttgaatta attaatattt atggggccag gcttggggct |
| 150961 | catgcctgta attccagcac tttggcaggc cttgagctca ggaattcgag acaagcctgg |
| 151021 | gcaacacaca cacacacaca cacacacaca cacacacaca cacgtgtata tatatatgca |
| 151081 | cacacacaca tcagctaaaa gcagcagaat acacattttc ttcaagagaa cacagaatat |
| 151141 | ttagcatgat atgctatatt ctaggccata aataaatctc aacagactta aaataattca |
| 151201 | gctaatacaa agtatattct gtaactacag tggaattaag tttgaaatta atagcaaaag |
| 151261 | atatctggca aatccccaaa tatttggtaa ctaatatcat acttctaaat gacccagggt |
| 151321 | caaataagaa atcaaaatag aaatttgaaa gtgtttgaac taaattaaac acacatttca |
| 151381 | aaacttttga gatgcaacaa cagcagtatt tagaggaaaa tttataatag tatagaccta |
| 151441 | tattagaaaa gaagatctca agccagtgat ctcagtttct acctgaagaa actagaaaaa |
| 151501 | caaaacaaat aaatccaaaa taagcagaag aaagtaagta ttaaagacca gagctgaaat |
| 151561 | caataaagta gaaaacagaa aaacaaaaca aaacaaaatt taataacacc caaaacttgt |
| 151621 | tattatacag atcagtaaga ttgataaacc actggtcaag actgattagg aatttatcaa |
| 151681 | gaagaaacga tttccttttt ttcatactgt atatgtagca cccaaaaata aataacttta |
| 151741 | gtataacatt tattcatcta atacagaaga cagaaatgag aaaagtggca tcattacaaa |
| 151801 | ttctacagat agtaaaagaa caataagata atatgataaa aaactcaggc caataaattt |
| 151861 | gacaacttag ataaattgca caaattactt atatgacaca atctaccaaa gttcattcag |
| 151921 | gaagaaattg ataatcttag tagctgtata tctattaaag aaattaagtt gatagttaaa |
| 151981 | aattatccta caaaacgtat tccaagctct gactgtttcc ctagagaatt gtacccaata |
| 152041 | tttaaggaga aagtaattct aattctgcgt aaacttttct aggaaattga agaggaggaa |
| 152101 | atatttccca actctccctg aggccagtgc tactctatcc aaaaccagaa aaagacattc |
| 152161 | caagaaaaca aaactacaga ccactgtatg caagaagcaa tcggaaatta aaaattgtaa |
| 152221 | aatatcattt aaaatatcag aaaaagtaat tatgaagtac ttaggaacaa acctgacaaa |
| 152281 | agtggctcaa gatttgaaca ctgaaaacta taaaacattg ctgtaagaaa ttaaagaggg |
| 152341 | cagaaataaa tggaaacctc atttgtgaat tagataaatt taacactgtg aagatattaa |
| 152401 | ttttccttaa attggtctat aattcaacag aatctcaatc aatacctcaa taggctttta |
| 152461 | aaaatgcttg caaaatttga caagctaatc ctaaaattta tatgaaaatg caaaggacct |
| 152521 | acaagaggca aactatcttt gaaaaagaag agtaagtttg gaagacatat acttgccctc |
| 152581 | agagctcatt ataaagctac agtaatcaag acagtgtgag actgaaataa agatagacaa |
| 152641 | agaaagacaa tttctaggtc atgagagtta tgtttcctaa aggaaggcag cagatgctta |
| 152701 | ttaactttcc ccgtgccctc aggatgcctt aaacagtggt tgccaaccac tagcagcttt |
| 152761 | agcaccactt ggaagctttt tagaaaagct aattactggg cccatcccag acctactgaa |
| 152821 | ccagaatctc tgtgggcaaa gcccaggaat ctgtgttttc acaagctctc caggtgataa |
| 152881 | tgatgtccat taaagtttga gatccattgc cttggccttt ccctgctaga aatcctccag |
| 152941 | gatttgggag acaggggaag aggtctggag agagtggcag aattcaacac cacagaatga |
| 153001 | agaggaaaat tgatggtctt tgtccatgga atccattaaa atatactctc attgctgcca |
| 153061 | ggctgagctg atgaataaaa aataagctaa tctcaaatca agccatcagg ctattgatga |
| 153121 | taattgtgtt ttagggtgag caaacagctt taccaagctc acacagttga gacaatttgt |
| 153181 | tgtttggaac tatcttactg taagatcctt aagaagaatg cagaagtatg aaaataaatc |
| 153241 | caggactcag aaatctttca actaaatctt taaaattctc tctctcatgc acacacacac |
| 153301 | atatgcacac atacacatat agtaagattt catgacacat gagacaggat gtgaagctga |
| 153361 | acagttcgat gaatgcctga gtggtaagat caccttgaag aaccagaagt gagggtctat |
| 153421 | tctagcbggg tatgagctac aaactcattt cttggggaac acatgaacac atcggggagg |
| 153481 | ctaagtagct tctgaggcta ctaatagaat ttcagtcatt tctcctgtca tctgaaaaat |
| 153541 | atatgatatt gaatttctaa aaatgggttt agtgtagtct agtcttactg tctaaaacta |
| 153601 | agatgtggct tattccaata accaaagtga ttccatccca tgtgagggaa aaaaaggata |
| 153661 | atggtgaatt aaaagcacct gcgtgtattt gttgctcctt tagaggagtc tctcttcatc |
| 153721 | aacgttaact ttctttccta cttttgatct ggctgctaaa agtctaggtt tagatctcaa |
| 153781 | accattgtcc ttctctgcat gatgtaatag ttacaattac agtctcaacc ctctggcatt |
| 153841 | taatgaagat gaaattgatt tctttagact gaaaacaaaa aaatcactta caatcaggat |
| 153901 | actgaatatg gtagcaagta caaggtccca gcttctagta gacttggcag actgtcaagc |
| 153961 | tcttactaag tgaccccacc ccatgccttt ttgtcttttc tgttgccaca caattacaat |
| 154021 | aatttgttct ctgaatgcca gagtactgac aaggaaagga attctgaagc tggctgagag |
| 154081 | aagtcgtgta aatctcccaa tctaggctcc attgactctt gtctctttct ttgtatggaa |
| 154141 | ctgattttat aatttctgtg gcaatgaaag actagtaccc aaaatgactg ctggtccttt |
| 154201 | gtttattgat caatctgagc atggactaga attcatcctc caattggtaa caagaaaaaa |
| 154261 | ttggatgggg aagcatacca ttggctaagg cagcacagca agtttgaatt tggctgcctc |
| 154321 | ttctttcaat attggttgac atcatcaaga gcagacattg gcgttggagt taagagtatc |
| 154381 | aagagagtta ataaatacca atttagggta atattttatg cctagtcttt atcttttggt |
| 154441 | gaagtggttt ctaatatcat ctcagactaa atcttatggg atatttctac ttgtagatgc |
| 154501 | taaggtatta agaaaaagct caaagtagtt gtgttttcct tatcttccta aaagtttaga |
| 154561 | ttaaagatgc caaaaatttg tgtgttggta tgtgtcaaag catttgaatg cccttgtaga |
| 154621 | ctattaaaac ctttgttctg taccaggcac tgtattgagt atattgcatg tatttattat |
| 154681 | tatatttagt tctcacagca accgtacaca ataattacaa ttccttcctt tcactaaggc |
| 154741 | atagggagtt taaataaatt agccacagta caggatggca taaaactgta gcagagccaa |
| 154801 | aactcaaaca tgtctctgtc tgactcaaaa acataggctc attctcctag agccaggcaa |
| 154861 | caacctgaaa gctcttgagt tgacttttct agttctgtgg atgaatgcaa tcatctcagg |
| 154921 | acccagataa acagaagtcc ttctttctta ttcgtatttc tcagtactag gccagtcagg |
| 154981 | acaaactctg ttctccatgg ccgtgatctt gaagaaattg agatttcatc caaaggaaat |
| 155041 | caggtattga aacgtgtgtt tatgtgtatt gattaatctt ctgcctgttt ctggaaacat |
| 155101 | ttgaagaaat ataacaaaaa gcaaatatcc tgaaactgcc taatgaacct gatctctctc |
| 155161 | tctgtctttc cctttctctc tcataggtct tttgcctgta tttctctggt ggaatttact |
| 155221 | aaataattat atcaggtgtc ttcttattgt acttgactgc tttcatcaag gtaatcttga |
| 155281 | ggacagagac ttcctttgtt agccagggca ctctagggga acagaaatta cacacacaca |
| 155341 | cacacacaca cacacacaca cacaggaact ggttcatgga ggctaaaaag tctcaaaatc |
| 155401 | tgcaggccaa aggcttgaga acccggagtc ctcattgtgt aagttccact cagagtctga |
| 155461 | agaggggaga agacagatgt cccagctcag agacctttag gcagagagag aaaattctgt |
| 155521 | cttgctcagc ctttggttct tttcaggcct ccagtggatt agatgaggct cacccacata |
| 155581 | ggggaaggag tctgatttcc tcagtctacc aatgcaaagg ttaatctcat tcagcaacat |
| 155641 | cctcacagac acacccagaa taatctttca ccaaatatct aggcactttg gcccagccaa |
| 155701 | atagacacat aaaattcact tccacacttt cttactaatc cgtgcattct ctccaataca |
| 155761 | gggcacccgg cctggcaagg agtaagtgct ccatatatat ttactaaatg acaagagatg |
| 155821 | agattatcct tagactatga ctcagatata gacaatcaat tattaatacc ctatcatttt |
| 155881 | tccccactct gacaacccaa actgaaaaat gttgggattg ccttttttga caatatgatt |
| 155941 | ttctctgaat gcagactcat gttttgacct tccctgtctg ttcctgttta gtattcatgc |
| 156001 | tggcttaact cattgattga tcaagtaacc attgagcatc tgccgtgtta acactctgaa |
| 156061 | caagatcctg taatatgaat ggtgtttagc ctgatgaata aagtgtggtg tctgtgcatt |
| 156121 | catttttata tatttattgt ttttaaccaa actttaccca tgtaaagaat aacattgatg |
| 156181 | gcaggaactt gaatttggat ctacgtacat gttccatttc agactcacca tgaaatgagg |
| 156241 | tgcttgcatt tttcattgct gattttacaa tacactttca caccttttgg gtaaaatttt |
| 156301 | gcaatcaatg taactgaggc agcatggtat gaaagcaacc ctggaggtgg aaatagaaga |
| 156361 | ctagaattta aatcttggtt atgccactta ttggttgtat gatttttgtc aagtcatttg |
| 156421 | acccgtgtct attcagagtt tctttgctag aatgaaggag ttaactagaa tttattgatc |
| 156481 | tcagtagtca gtctcctcag gcacagataa atcttggata tgtatcctga gatagctttc |
| 156541 | ccatttccac attgaagaca acattctatt ttagagcaaa aatgaattat attatttaag |
| 156601 | aatatgagtt tcagaattca gactcatgct tacgggtttg aattctagtt ctgccacttt |
| 156661 | cttactatgc agactttagc aagtcagtta atgtttgtga gcctcaggtt tctcaccagt |
| 156721 | aaagcagtgt gagtaataac tatcatatta tattattaca agcataaaat gaaataagag |
| 156781 | tacttggcac aaagaaggat cttaattttt agctttttaa aaaagctgta tttgtttata |
| 156841 | tgcagtatgt atatatatat tctcaattct attattatag actatattta ttatgttttt |
| 156901 | cataaagtca ttagtttcac tcaggaatta gttgaccctt gtgagataca tggtttgacc |
| 156961 | acttttcact ttgatgttct tcagaaaata agacaattag aaatctctga ttgtaatctt |
| 157021 | catgtaaaat attttacagt catggctaaa atgatcccag gagtggaatc atacacataa |
| 157081 | attcttttct ttattctaat aatgttacct ttagtgtcta tgccaagcaa gtatgttatg |
| 157141 | tgttgagata aaaattctta gtatgtttgt atgtgacatt cccaactccc atcccactga |
| 157201 | agtctatatt tacctagttt ctttcttttg ttttcctcta acattcatgc tattttaatc |
| 157261 | aatgttcttt cattttgaag cccccaataa gtttgacccc aaagactttc agtttgcccc |
| 157321 | tgctattcat aagcatactt atgacaatgc atgtctccca ttcttttctc atatttacac |
| 157381 | cgatcttttt attgggacaa atgggtttgt tttttcatgc ttgtgtttaa tatctcactg |
| 157441 | agttcagcat aagttattaa acataatttt ctgttttctc tttctccctc aagactttct |
| 157501 | ttttctctag acctttattt cctaactcca gagtatttgc ccaggtctat aggcccctct |
| 157561 | ctcttttgct ttcttaccaa cccatccacc ttcttctgtt tcctattgcc caactggctt |
| 157621 | cacttaaaca agagcatttg ttatttgtac agagtgcaac atataattgt cctactctgt |
| 157681 | ttggttgcta taacaaaata tcatagacta ggcagcttat aaacaacaga tatttatttc |
| 157741 | tcatagttct ggaggctgga aaatccaata tccaggcact actggcagat ttggtgtctg |
| 157801 | gtgagagcct acactctggt tcatagacag ccatccattt gcbgtgtctc catatggtag |
| 157861 | aaagaatgaa taaagtttct ggagtcttct ttacaatggc actaatccat tcatgaaggt |
| 157921 | ttcatcccat gacctaacaa taccattgta ttgggggtta tcttgcaaca tatgaatttt |
| 157981 | ggggaacata agctttcaat ctatagtaat gaagtaggaa attttccctg atcctttcat |
| 158041 | gggcaggaac tggagtatgc ctatttcaca gaggtcttat cagataaaaa aattctccat |
| 158101 | taaatggcaa aacaagtaac catttgggat tcctgggtgt acagtgccta ctattccaaa |
| 158161 | atgagattat tgaggctaac atgacccttt gtatacaaag acgggggcag aaataatatt |
| 158221 | tctagcaaat ccacacatct caatcacatt taatttcaca aaatttactt ctaataagca |
| 158281 | ttgttccaac taacaaacca tctggtgcta gtctaagata gagatggctg aagtgctcct |
| 158341 | gattccagta ttttaggtgt taaatctcca ttctaacatt gtcaatcctt aactggagta |
| 158401 | ctcaaatatt aactaacttc ctctaggata taaattaata tgtataccac attctatgtg |
| 158461 | aattatttta attatattac atgccatata caaattatat ataggtttag tcttcctgaa |
| 158521 | tattgcaagt tgagtctgtg agagaaaaaa ttaggatgaa ttgagaataa ctagaattca |
| 158581 | gaattctttt tcattaagtg ttgactgtgt taactcatat tcccggttag gtagcaagtc |
| 158641 | ctatgaagtg tgattttctc ctaacagcta aaaagcagaa aaaaaatgga tttattctag |
| 158701 | ttaatgactt gctttttcta tttttataga aaaaatgaag taaattattt cacttttctt |
| 158761 | gatgctatta ctattttgtc aggaagatag atcttctaaa ttttaggaaa ctatgaatct |
| 158821 | aagttctata ttgctctcat ttcctttttc tctttgtctc tcttctgttg tagcccctcc |
| 158881 | ctcaccgcta agccttcacc ctgactctac cctgccccca ctgccttttg ttctttggaa |
| 158941 | gacactcatt aacatttgct aatgtatgct aatgagcaat aattagcatc tctaatgtga |
| 159001 | acaaaagcag tgctacaaag gaagggtaaa gatgtgtaca gagttcagcc agccttttat |
| 159061 | gcttctttat gccttaggaa caatcctctt ctttcagttc tttcacaatt ctttagtgtt |
| 159121 | ttttgtttgt ttcattttgt ttgcttatcc atgaaccctg aaaaccattc ttcttcttga |
| 159181 | gttagaatat tgcaaaaatg ctacggagga cagaaagatt cctttttgtc ctaaataacc |
| 159241 | caaggatata agcacagctg ctaagaggtt ataatgcaca tttgtatttt catctggttg |
| 159301 | ttattttggt atcctattac tagaaaaggt tttaaccaga aaacctcaca caagataatg |
| 159361 | tatttcattt taccacactt atttacagtg gttgcccttt atggtggtaa ttgcaatgtg |
| 159421 | aaagcataca ctctgaatat atattatgac agcatggtta aataagaaat gttccctccc |
| 159481 | tcaaacaatc taaatgcaaa gaaaatgcaa gactcaccac ttactttttc ataaccacag |
| 159541 | agggctctaa atgtctttgt aaatagctat ttatttattg aatatgtata gttttttttt |
| 159601 | tgaaaaagcc agccctccaa accacataga gtaagagctg tttttgttct ttctgtagac |
| 159661 | tgatggtgtc gtgaaatacc taattgcagt ttctcacaaa tactccagca tgtaatttgc |
| 159721 | catttgcaga accaggcttc acatttcata tttgctcttc cagctcaggg gtggtagaat |
| 159781 | ttggaaacat accagcaacc cacttcactc agcctgtgtt taattataat ttcacgtaca |
| 159841 | ataaaagcag aaggaaaacg tgcaaaaaaa ttggtgtgca tccctttccc ctccttctta |
| 159901 | cgtatataaa aaagatttat ttccattgta aaaggataga atggggatct ttattttgag |
| 159961 | tagattttct atttttgttc tttctgtttg cctcagagtt agtgtcctct ctctgtgacc |
| 160021 | tcagagtaaa atgtgtttct ccataggctt catcctgttc tatttatcaa ctatattata |
| 160081 | tttatcaatt attttatatt gtgtttttaa taaagcagac atcagcagct atttattttc |
| 160141 | tacttctgag ggcctaaaga gtctcagaaa atgagtgtaa tctagaaggg aaaacattga |
| 160201 | agagtgacag ctattataac agttgtgatt tttgcctgcc tccctctttt ggattggtga |
| 160261 | gtcctaccaa aaaccaaaac tattattctc cttgtaattt tgaaaccaca gagattgaaa |
| 160321 | ataactattt agtatctcca agatattata atgtttgaaa atatgaccag ttttgggggc |
| 160381 | atttattaca aaagtttcca gtagggtgtt tttgaatgtc tgtgccctct ggttttcaca |
| 160441 | aaactggtct taaggaagat aagatactgg tacacacaga agaaatatca gcctacgtct |
| 160501 | gtctcattaa atcttatccc cagatacttt gatgatattt aagtgtaatg atctcattct |
| 160561 | gtgtttctca ctcctgataa ttttttaaat gtcttatagc ttacaggttt attaaggtat |
| 160621 | ttgatttatt taagaactac ataattattc tcttcattca atggcttctg aaatattatg |
| 160681 | tttaattcta tcatttgaag gacttgggat tgttataaac aaatggcacg agcttctttt |
| 160741 | tgtcctgacc tagaagtagc aaagataaag ggagctgctt agttaacctg agtgagattt |
| 160801 | aataatcttg ggtatatttt attctagagt aagaagaatt gcaccctatt acttttattc |
| 160861 | agttttgtat gcagtagctt ctgttgtcta cttcacaacc accaatagtg ttatcataaa |
| 160921 | ttcagagtgt tgggctgtat tagcccattc ttgcactgct ataaagaact acctgagaca |
| 160981 | gggtaacctg taaagaaaat aggtttaatt ggctcatggt tctgcaggct gtacagaaag |
| 161041 | catggctgct ttctgctctc cagcctcctc atggaggcct caggaaactt acaatcatgg |
| 161101 | cagaaggcaa agttgaggaa ggaggtacct cctacatggc cggagcagga ggaagagaga |
| 161161 | gaaggggaag gtgctacaca cttttaaaca agatcttgtg agaactcact attccaggaa |
| 161221 | cagcaaaggg aaagtctgcg cccatgatcc agtcacctcc caccaggcac ctcttccaac |
| 161281 | actggggatt acaattagac atgagatttt ggcacaaatt tgaaccatat cctgggccat |
| 161341 | tctctatatg gagatgatgc ctgaatatga tttggtgact cagtctagca tccaagtggc |
| 161401 | tcattgttgg gcaattcttc tttagtcttc tttagggcta attttactca ttccttttga |
| 161461 | aaattggatt gatttacttt ttgtttggtt ttaagaaagc aaaatatcta aatgctgttc |
| 161521 | ttggcatagc ttatatacat atacatgcat acacatgtgt aattttcttc agatcaaaat |
| 161581 | atgcatcccc atcattcgta ttgctataat cagaaattag taatttgagg ggcaactgaa |
| 161641 | tgaccttata cctagagttt tcagagtata atattacaca gtgtgtattc ataagaatgc |
| 161701 | tgccttttaa agaggttttt ttttaaaaaa atcatagctt attttttata gtttcactgg |
| 161761 | tctgtcctcc cttttctctc tttttttccc ccttgagaag agagatttca catttttgtt |
| 161821 | aaggggctca taatttcctt atgaaatatg acaaatttca ctttatctta aaagcaaaat |
| 161881 | attaatagta cagacagctg aaggtggtct gagttaacct gagttacaca ttttatatat |
| 161941 | atatatatat atatatatat atatatatat atatgttaaa aatatttatt tgtcatatgt |
| 162001 | agcaagacta aaatgcaaaa ttttgcttta tgcttattct tttccagtat catttactgt |
| 162061 | aattctcacc ccatatttat ttttttactt ctctttctgt ttctctcctt tctcctctca |
| 162121 | ttctttttct ttcttttctt ctccctaccc catcttttag ttccatactt ttgaattgct |
| 162181 | atagtggact caaatctaag caagaggtat attagtaagt tgttcagtct tttaacaaag |
| 162241 | tatcagtcag agaagcacag cccagggata aaccaaaagc agaaataaaa ctgaggaatt |
| 162301 | aataatctaa ttcgtgttgt tttctttaca ttataggtgg ttcatttcat agcctacatc |
| 162361 | ttcaatttct caattctcat tagatctttc ttaacagcat ataagaccat tctaaatagc |
| 162421 | aaaaatagct gtatcctttt actcactgcc catcagttca taaagccact tgaattcagc |
| 162481 | tttttgccca cacaactgca ctaaacatat gcctgccaag atcatcaata tctctacatt |
| 162541 | gtcaaatcca gtggtcaatt ttcagtccac attgtactta gcaacttatg atacgtctct |
| 162601 | gatgcacatc ttctgccatc cacaatctct gactttcctt ctacttaaga gctgattgtt |
| 162661 | catttttgat ttcttcagtt tgttcttctc ctgttcctca ccattttgag tctccttttt |
| 162721 | ctgttttgtt taccctcatt ttctttgtta tctcatctgg tcttatggct ttaaatacaa |
| 162781 | tgtttttgct gataattccc taattcatat ttccagtcca gacctttctt ctgaactcca |
| 162841 | ggctcaaata ttcaactgcc tacttaatgt catgcttgga tgtctagaca gcacctgaaa |
| 162901 | tccaacggat ctgattaaag tcctgatctt taacagctcc tacctaagct attctcccta |
| 162961 | cagtcttcca tcttcttttc ctgtaattcc attcttcagg tgctcaagca aagaacctgt |
| 163021 | tgctatcctt gatttctcta tttctctcac atcctacagg gatgacatgg gttcatttat |
| 163081 | gcctgtcatg ccactgatta acatccctat caagaaagtt tagtctgagg taaaaacatc |
| 163141 | caatttgtat gagaagggcc aacattgaca ttcttcgtac acaaaaaaga atcctgagaa |
| 163201 | gaaacacaaa agagctaaat aatacattgc tgttgcagct taggtaggct ggagaagaaa |
| 163261 | cccatacata atctcaaacc ttactgttat aaagtcattc tttttttttt tttttttttt |
| 163321 | ttttgagacg gagtcttgct ctttcgccca ggccagagtg cagtggcgct atctcagctc |
| 163381 | actgcaagct ccgcctcccg ggttcatacc attctcctgt ttcagcctcc cgagtagctg |
| 163441 | ggactacagg cgcccaccac cacacccgga taatttttta tatttttagt agagacgggg |
| 163501 | tttcactgtg ttagccagga tggtctcgat ctcctgacct catgatccac ccgccttggc |
| 163561 | catttttttt tttttttttt taagagggag tcttattctg tggcccaggc tggagagcag |
| 163621 | tggcacaatc tcggctcact gcaacctctg cctcccgggt tcaggtgatt ctcctgactc |
| 163681 | agcctcctga gtagcbggga ttacaggcgc ctgccaccaa gcccagctaa tttttgtgtt |
| 163741 | tgtagtagag gcggggtttc accttgctgt ccaggctggt cttaaactcc tgacctcaag |
| 163801 | tgattcatcc acctcagcct cccaaagtgc tgggattaca ggcgtgagcc actgcgcctg |
| 163861 | gcctataaag tcattctttt gcatcaaata gtaagactca ttaaaggcag tggtattttc |
| 163921 | ttttactgtc tacaaaccct tatccaagag cattttctct atattccttt gtgagcacac |
| 163981 | aataaatggt aattcaataa gtgaattttc agtttccaaa ataataacct ggagctataa |
| 164041 | acattgcttc aagaagtaaa tgtgggtcag agaaaaaggc aataccattc aactcttctg |
| 164101 | gccacttggt cactgtgtta tcctgaagaa gcataaacta ctttcagcat tatagattta |
| 164161 | aaaagaagcc ccagaacaac atccactttt ctgaacgact cctatgcctc tgaaatgtga |
| 164221 | cattttgctg ttgtttcttt ttactgtttt taattactga gtctatctgc atgaaatgag |
| 164281 | gatctgggtt gcctaggagt tcatctgctc ctattgcata ttcctctttt aactacacat |
| 164341 | acttcaatgc agttttgctt gctgcctcca acattttgac agacttcccc tcctcttcct |
| 164401 | tcactatcct cccatcactc tccaggagac aaggttattt ttaaagtgca tgatcaatgg |
| 164461 | gcattgggga gcagctgagg catgaagaga tcatacgatt gggaataagg tactagggac |
| 164521 | tgctgagaga aggaatggca acatgccatc tcagacattg tctgagaaag gaaagggttt |
| 164581 | gtagctccca ttcaagtatt ctgagctatc tcttcacaca ggtttttgtc ttggagggag |
| 164641 | ttcttaacat agttctttag cattgcttta agttgaagga gatataggcc agtgctgata |
| 164701 | gttacttttc aggccaaata atgttccatt aaaatcgagc ttgctctgag gatcttggta |
| 164761 | gtgtcttaaa tctcctggtc atagtgctgc tatttggttc ttgatgctta tgacaccttt |
| 164821 | gctgagggtt tgaatcaaga acaaaacaag tattatcaca atgactataa aacatgactg |
| 164881 | ttccacgatc agacaaagga atgcbgttaa gcaaagaagg cacctaagga gaatgtggag |
| 164941 | atgaccatgc acagaccaga atcactattg gaaaaccaac tcaagaggag ggactgggtc |
| 165001 | atttctgcag cagtcttcat tgcatcactg agaaattgca gctctgtttt agttctctac |
| 165061 | ttgccaccac agggaatatc ttcccttatg gacttttagg ttttatttaa tggttattta |
| 165121 | taaactattc cttctgtgcc ggagaagatg attcatgact ctagccctgc tttttctctc |
| 165181 | ccgtggttat tagaggaaac tgagttgttg cttatctctt tagcttggga taccttattc |
| 165241 | tgctcttttt atttaatata taatctttcc taaagatttc cttatatttt tgttcattta |
| 165301 | gtaggttttt ctgttttatt taattaactt tatattatca cattctagtg aaataatcat |
| 165361 | cacaaatcat cctaatacat cgtgcaaatg ttttaaaaat gtgctttggt tgtgcatatc |
| 165421 | gcaaagtagg ctgtgggtca gcatttgcta tttgtcctca gctgtttctt ctttatatat |
| 165481 | aactgataac accctgctcc aatgttaatc ttatatcatt aaaattcttt cctagagaat |
| 165541 | ccttctttga ctgaagaagc ttagtggtga cattaattgc aaataaaaga ccatgaccta |
| 165601 | ttgtagaatg tggaatatct ccttagaagt gtctgccttc attattgcaa actttcagta |
| 165661 | gtgacataaa gatctttgtc ctgtgtagtt tcagttgcag ccagcaacag aaggcatatg |
| 165721 | ttcactttga acaaaagaac atttattttc acattctgtg gctgatgttg agtgataaca |
| 165781 | tttttgcata tcctaaaatt ttttacccag atgcbgttta aaggcaaaaa gtgcttttac |
| 165841 | aataatattc tttgtgaatg gaatttacac tcttggcatg tgttgccttt tcaatatgcc |
| 165901 | catcaagttc tgaataataa cctttgacag tgagtgaaca gaaacaaaac cagaggttgt |
| 165961 | ttcatctgtt cacagcaaca ctgtttgtca tctttgcttt tttggaaaaa tgtcattcca |
| 166021 | ccttaaataa aaatatgata aaattcatgt tttataagca agttaataga cttcgattat |
| 166081 | taagaaatat tttaattttt ccaaggcaag aaacctgtga tgaatgaaca ttaagttgac |
| 166141 | aaaagaaaaa aatatgccaa taaatatatt ttttcatatt actgtagtgt caaaaatttt |
| 166201 | ataaaacttt tatatgcctt tataaaaatg aagcaaagct aagaagaaga tgctgtatgt |
| 166261 | gttatggggt gaggaataaa cagaaaaagt gtcctttgtc ttggaaagaa ctgtaaaatc |
| 166321 | acctatctcc atgttgccaa agggaatcac atattgggaa atttaggcta tattaatata |
| 166381 | atacgtataa aaataattga aatagaaaag atctggcttt aagcattttt ctaaagactt |
| 166441 | tttcatgctt ggtaaaagtg tttggaataa attattctct atttttaagt agttaatttg |
| 166501 | gttattaacc taattggtat gggcatgtgg atatagcttt attaacaatt caatttctca |
| 166561 | tgaaataaaa taacccgaca aaccccaagt ctacttattg caatacagaa tttattttgg |
| 166621 | ttgaagttat tttgaattga gctgatagct tataactttg aattgggctt ataactgtta |
| 166681 | tgttttgcaa atccctgaag attatgaaat agaaaaatct ttgcatggca ttgtataaga |
| 166741 | tgctgtagct gtatatcttt aataaatcgt gctcattttg gcaagtttat ggaaaaattg |
| 166801 | tattaactgc acaaaaacat tatgataaaa acaataaaag taaaaaccac ttactaacaa |
| 166861 | ttgtacaggc atttaaataa ttgctgtact gttttctttc ctcaacagta caaatcaaag |
| 166921 | tggaggaaaa agaagacgag actgaggaaa gctcaagtaa gtggtgatgg gccacttggg |
| 166981 | atacatttcc tatggaatac cagtaaactc aaagaattta tcctcttgtg gacttgagct |
| 167041 | accatatgta agcagaagtg tgtgtggata acttttcatg gtctcacagg aggaatcaag |
| 167101 | ttcagcaaac agaatcccca ctctagagtc tatccagttg gcactataag ggccaagact |
| 167161 | gtgttccctt gattgcttgc tgaaaaatca acaagcaaaa gacatgttaa taggagaaaa |
| 167221 | ggcatataat ttcatagaac ttgtattcat taaagcctta ggatgaagac ccaaagatag |
| 167281 | agaaaattgt ccatttttat gcttaggttc aacaaagtgt ggatagcagt gtagaaatat |
| 167341 | gattggacga aaagggtata atctaatgtt tacatagagt ggggaaacac aacaaggcct |
| 167401 | gtctgcctag attcttcttg gcctctctaa gcatgcattt ctttcttctg ggtatggggc |
| 167461 | agggaccctc tttggaatga gggtcttatg atctacactc aaacaaggta ggtcggatga |
| 167521 | tttcttgatg accagttttt acacagacag ggcagaggga atgttagagt agtattttta |
| 167581 | ggtttcatgc atttgagaag aatgtttatt ctgttgttat tgggtagaat gttctgtata |
| 167641 | tgtctgctag gtctgtttga tctataatgt tgttcatgac tgctgtttcc ttattgattt |
| 167701 | tctgtctaga tgttctatcc attatggaat gtaatgtagg gtattgaagt ttcctactat |
| 167761 | aattatattg ctgtatattt tctctcttga gtttggttaa tgtttatatt taggtgctct |
| 167821 | gatgttggat gcatatatac ttataattgt gatatcttcc tggtaaattg acccttttat |
| 167881 | tactatttaa tgcccttatt tgttgcttat gacagtttta tactaaaagc atattttgtc |
| 167941 | cgatgtaagt atagccaccc ctgttctttt ttagctacca tttgcatggc ctatcatttt |
| 168001 | ccgttccttc actttcaccc tatgtgtgtc cttaaatcta aagtgaatct tttatagtta |
| 168061 | gcatacagtt gtttcttttc tttggtttct tttttaattc attcagccac tttatgtctt |
| 168121 | ttgattgaga agttcaatcc acttaaattt aaagttatta ttgatagagc aagactaact |
| 168181 | tttgccattt tgttcattgt tttctgtctt aaagctcttt tgtccctttt ttatctcatg |
| 168241 | ttattctcct tcatatttta ttgattttta ttatagtgat atactttgat tcctttctct |
| 168301 | tttcttttgt gtgtcttcta tagaaatttt attgtggtta ccatgggact catgttcaac |
| 168361 | atcttataat tataaaagtc ctattttaag tagaaaacaa tttaacctca attgcatact |
| 168421 | aaaactctac acttctactt tccttgacac ttaatattat tgacatcttg agttacatat |
| 168481 | ttttatatta tgcatccatt aacgtatttt tatagttact gttaatactt ttttactttt |
| 168541 | ataatacaat taaaagttac caatatactg tcattaaagt attatagtat cctgtatttg |
| 168601 | tctacatatt tacctttacc agacagtttt atattttcat gtgattttgt gttgctcttt |
| 168661 | agtgtcctct cttttcaact tgaagaactt cctttagcat tacttgtaag aaaagtctag |
| 168721 | gggtgattaa cttaggtttt gtttgtctga gagtctttat ttctctgtca tttataaagg |
| 168781 | acaaacttgc tgagcatagc tttcttggtt ggcaggtttc ttctttccaa cacttttaaa |
| 168841 | atatcatcct actttcttct ggcctgcata atttctgcta agaaatgata gtcttatgaa |
| 168901 | ggtttccttg tacatgacaa gttgcttttc tgtttctttc aaaattctgt ctttgtcttt |
| 168961 | gacttctggc aatttaagta taatatgtat tggtgtggat ttatttgggt tcacataatt |
| 169021 | tagggtctgt gggcttcctg aatctgaggg tccattttct ttcccagatt tgggaagatt |
| 169081 | ttggttatta ttttctttga atagtctttc tggccttttc tttcttttat tctccttcag |
| 169141 | aactcccata gtgtatatat tggtctgctt gttgatccct cataaaactt tcttgacttt |
| 169201 | ttcttattct ttttatggtt ttgctcttct aactataatt tcaaacatcc tatttttcaa |
| 169261 | tttggtaatt ctttcttttg cttgatcgag tctgcbgtta gacctctgta gtgaatttct |
| 169321 | gttactctat tcttcagtta ttgtgttcag ttattgtctt gtactcttca atatttatct |
| 169381 | ctggttattt ttatattttt atttctgttg aaactcatac tttgttcatg catttttctt |
| 169441 | ctgaatttgt tgagcatctt tatgagttat ttttaatagt ctgttaagta attcatatat |
| 169501 | ctccattata ttagggctgg tttctggagc tttatcttct gttgtttggg gtatgttttc |
| 169561 | ctgtttcttt atttttcttg attttgtttg tttgttttgg tatccatata ttagaaaaaa |
| 169621 | cagattcctt tcccagcctt caggagaagg attagccttg tacaggagaa gagtttcacc |
| 169681 | aatgaggcag gccagagatt cttgggcctc tcaaacattt cctgtgtatg catcgtctct |
| 169741 | ggacttgtgt gtgtaaattc tcaataagag ggatttttca gttgactttt tctttttaag |
| 169801 | aattcataat ctcttgttcc ctccagtgtc cttctgtcat aacatgtgtc ttctgaggaa |
| 169861 | gcagcatgtc ctgagttctt ttttttgttt ccagtagtcc ccaggcatca acagtatgct |
| 169921 | gggtcctgtc catgccctga gactggtcat tcccttaggc agcctcctga gaagtctgaa |
| 169981 | cacagggtat atatttcagt cttttatttc ccatttcagg ggcaagtcaa gggctggaat |
| 170041 | tcttgccaac tgtgtcatgc tgaactgggg gaaggggctc tggtgaatga gtacaaacta |
| 170101 | attcaaaact tcacctttgt tctccatggc cctgggccat ttagaatatg ctgggtccta |
| 170161 | ttagtgctct aggagacaag tgggacagaa gccagtccag tcatttttta gaaaagttgg |
| 170221 | agtgttagat gcactatcca actgtttttc tccccagaga ggtgcagatt tggtgtttat |
| 170281 | catctggtta ttctgtgcta aattggggga gtggctattt gagtgaggca tactattcaa |
| 170341 | acggctatct ttgctctcat tggttcctaa ccagaggtct tttcctgtca gtgctcagat |
| 170401 | tctggcaaga cagaaaccag tcctttggga acctccagaa aaatcagaat gttgaacaca |
| 170461 | tggttcagtt gttttctccc ctccccaggg agagactggg agatgatggt tttctcccag |
| 170521 | ttgtatggta ttgtgttagc agagagggat tatggtgtga gggtgtgtca gatttttcta |
| 170581 | ccagctacct agctgatttt gtgttcatct agggttcagg cacctttcaa ctagtttccg |
| 170641 | aatttctcac gaagggagct tatctccaca ttgtagttga acttttatgt ccatggaggg |
| 170701 | aaggcgggtc cagggcttcc tattttgtca tcttgttgat gtcatcccag tagtttagtt |
| 170761 | ttaatacaaa aaatactaaa attcagagta ccaagtagct gcttgggttt gtgagttatt |
| 170821 | agtgggtaaa ttgtttcttg cctcctgtca agtttttgtc ctttctttaa ttagtgacta |
| 170881 | atgtggctaa taaaagcagg gtaaatattc agaatgccat gatggggttc aaataacata |
| 170941 | catttgccac aatattaaac aaacagaaac tcgttcttga tttggatgac agaatatata |
| 171001 | tacagttttc caagcccagt tagaaaagtt attatgggcc gggtgcgata cctcacacct |
| 171061 | gtaatcccag cactttggga ggccaaggca ggtgaatcac ttgaggccag gagttcacga |
| 171121 | ccagcctggc caacatggtg aaaccccatc tctactaaaa atacaaaaaa ttagctgggt |
| 171181 | atgatggcgc acacctgtat tctcaactac tcaggaggct gagacaagag aatcgtttga |
| 171241 | actctggagc tggaggttgc agtgagccga gactgtgcta ttgtactcca gcttgggtga |
| 171301 | tagaacaaaa ttgtgtctca aaaaaaaaaa aaaagtgaag ttattctgtt ttttactctc |
| 171361 | tgctacctga catttagtaa tatatgtaat atttagatct tatacctgaa atatcaataa |
| 171421 | aaaagtttta acaagtggta attaaagata attttgaaaa aataaaatga tgactcacac |
| 171481 | gtatgtgaaa agaacatatt taactaataa aacaacttgt gagatgctat aatcagttca |
| 171541 | aaggaacgtc caaagaatag atacatttat agataataaa tattgaaatg attgtacaaa |
| 171601 | tgaaggcaaa gttgtttttg aaagggggct tgtctctcct ttggaaagag ttcatttgga |
| 171661 | tgtctacaga taaaaagtgt tttgtattgc tatatatggt tatgtataat tttcaaagtt |
| 171721 | gtcatatagt tctcataaaa tcagaatcag ataacacaga aaggtatgct gcagataatt |
| 171781 | catagttttc cattgaaata taattttttc tctggggtag cctctattta agagagtaat |
| 171841 | gataactgta atatttaatt ttttacagta gtttatgcta aatgttagaa gttaggagca |
| 171901 | atagaaatga aactggaata gtaatgttat gttataaact ttgtgaagga aattttatct |
| 171961 | taattatctt aatttatctt aattatctct ctattcttta cctggaagca tcctaaaaag |
| 172021 | ttatgaacac atgaggcatt cattgttgtt gatgatgaaa caaaataatg ctgtcattat |
| 172081 | tctcaatttt acattaaatt tatgttacct atttctattc ataagaaata aatgatcaag |
| 172141 | caaatgtgta tactactttt ttttttaaag aaaagaacaa aaatattgag gaaaaaggag |
| 172201 | aaaaatattt agaatcaaga atgaagaaaa tttaaattga gaggaacctc aagggttgaa |
| 172261 | tgaatttctc caaaattatt ctagtttgta gcacctcttt aacgttcctc ttctttgttc |
| 172321 | taagatagaa gtagtaataa atatgaagtg gttaatcact caacaattat gtaatggaat |
| 172381 | ggtaatctct aaacttggac ttatttgaat ggtcttagag gtcatggtat gatattaacc |
| 172441 | accaagctat ttagttactc aagtaaatga agaccagaac ttcttcaaaa acttattaaa |
| 172501 | atttgggaat ccacattcac ctatttgagt cggtgaacca caatggaaat ggaaatctag |
| 172561 | atacatttat ttaaatgatg ggctatgaaa tatgtattcc atgatgcatc ccatcacgat |
| 172621 | gtggagaatt gctagactgt gcbgtcagat gtgtaataat agttattaaa aactaagaat |
| 172681 | aagcttggta gtgggaggaa gcactttctg acattactca ttgaatcaag tgctatgttt |
| 172741 | aggtgaagag gaagaggagg tggaagataa gctacctcga agagagagct tgagaccaaa |
| 172801 | gaggaaacgg accagagatg ttatcaatga ggatgaccca gaacctgaac cagaggatga |
| 172861 | agaaacaagg aaggcaagag aaaaagagag gaggaggagg ctaaagagag gagcgtaagt |
| 172921 | tagttctgat attctttcaa aagttccaat gctgaagaag atcactcctt tcagatactc |
| 172981 | tttgacctta aaattggtaa ttaaagctac ttttagaaag agagccttaa tctgatgatg |
| 173041 | ctattttatt ctacttaatt aaatgtattt taatggttaa aatagaattt taaaatgaat |
| 173101 | atatattaaa aatggaccat gagaaattct cttatgaagg atctgaatta tcataggtcg |
| 173161 | ttgacatatt acagttaatt taaagatttt aaaatttccc cactaattgt atgcattgtt |
| 173221 | atctaggtta agtagaaact cctaaatagt gagcaaacat cactttatct tacagagcag |
| 173281 | gggttggaaa attatggcta aatccagccc aagaggtaag aatggttttt acagcttcaa |
| 173341 | agggtcatta ataaaaagca acaaatatgg gacagagttg tacatctcac tcaaagctta |
| 173401 | aaatattaac tatttggccc tttatagaaa gaaagtgtgc tagcccttgt tacagaggat |
| 173461 | ttgggttgtc attttctttt taatacaact caatatcttt tagttccacc aggaaaacat |
| 173521 | tttagtaaac gctgactttg tgtagaataa tgcattaaat gcbgtttagg aaaaatccaa |
| 173581 | gaaaatactt ttcttctttt tgctttttgg aaatttgcag atagcaggca ggtaggaaaa |
| 173641 | atgtacgtgt aaaacacctc taattaaaga gaaaacctag accccttgga aagaatgcaa |
| 173701 | gagaacagcc aacctgttcc acaactcaca ggtaataggg ccatgcgagt cgccatcagg |
| 173761 | tggtggaatc tcctccaaac actcctagga ctttgaagca tgaggaactt tctaagatgt |
| 173821 | cctcccaaga tgacacactt ctatactcaa tagctctaac tattgtgtta ttccttataa |
| 173881 | atgattgtca ttaaaaacaa ctgaatgaaa aatttgtcaa gtagagaagc ttagcattaa |
| 173941 | caaaaaatgg tttcttttag aataagaata acaccatcta cttcatgtct aagttttcct |
| 174001 | cctcctcttc cccagctttt cctttctccc ttttgaatac caagacaaaa cttgctttta |
| 174061 | aattttgact ctagtatttt ttatttatat ctgcttttaa agtttcttta tgctttattc |
| 174121 | ctttcaaaaa taaaatgcaa cacattttat gtcccatgta ttactatgat tgttagttcc |
| 174181 | attattatgt agtaatagtt cctatttagc aaagcttatg ctttccaaca gtggtatttc |
| 174241 | attgagaaaa catctccaat tgatgtatgc atgatatagg ttgtgttcca aaatttgctt |
| 174301 | ttcctctcta tttatgccgc aaaaaaatcc ttttaaaaag atgagttgga caggaattcc |
| 174361 | tctgctctct tcgtctgctg atggccctca tggcaactgc tcaacttcta ggaaatgggg |
| 174421 | ccatgtgaag caactggatc ctgtggctat gacagtgtct cttcctagct ggcctcactt |
| 174481 | tcattttgat cttgagaatc aagatccctt acctcccact cttccacata acccaactct |
| 174541 | ctccataggt tttgcaaatt gtatttttgc tgcgacaatt tgtgcatcta ttataaatag |
| 174601 | tatcagaaat tagttgtgat tattgatagt tctgttctct attttagagt ctattttcta |
| 174661 | ttgttcattt tgaagataaa catctttttt ttaaaagaaa atcttagata atatttttga |
| 174721 | gaaagattag tagccaaatc agggcacatt tatgtaggtt aattaattca gggtattttt |
| 174781 | tatttaacta ttcaagtaat gcctcaggga atttcccttg cattgacaca acatttgaaa |
| 174841 | ccaactaaaa ccaaggttgt aatgctaact atctttggag gcagttaaaa aaacactgcc |
| 174901 | ttgttagaca ttttggagta tttccagtaa tattacaggc ttttcctgac ccagttttaa |
| 174961 | gtggataagt aatatattca tagcagtgag aaattcagca tattatgcct tcttcttcct |
| 175021 | aacaactgtt gtttaaatat atcaaactta agatagaaaa ccattcttgc acagattagc |
| 175081 | ctgaaaattt agatgaggca tgcctatgcc ttttaatatg attagccctt tctttgagtg |
| 175141 | attctagaaa agcaggtttc ctgtaatgct gattagaaaa gtattttaat taccttcata |
| 175201 | tttgagggta acttatgtgt caacaactgt accctgtaac acagcctgat cctcctggtg |
| 175261 | aaataaacaa ttaacaacag agacttaaaa gatataccaa tctggggtca tttagtacag |
| 175321 | gaataaacgt ctttccatac cataataatc tcagaggtag ttgttatatt ttagtgtact |
| 175381 | ttgagcactg ggtttatgat gataaccctg agcaatccag aaaagttttg tatacatatg |
| 175441 | ttcattcaat aagttattta cccagctcta cttttgtgca acctaatttg cttatatctt |
| 175501 | tgggatactt ggatatgaac atgtaattgc ccacaatctt gcaatcttca aaagtccata |
| 175561 | atatagtaag ggataggcca cttagataca aattattatg acataagaaa gtatatgtgt |
| 175621 | cccagagaaa tacaaacaaa gtagcttgga gtctcagaga agtgaaaact gttctcacag |
| 175681 | ggatagttag aaaagggatt cttaaagaca tttcatatga tttttaaagg acttgaaatt |
| 175741 | tggacaagct gaacttataa actatttaat tagtattaga attgtttcta ttaaagggtc |
| 175801 | tctgaaagtt ggcctgtagg tttagttacc tggcaaatcc acacatcaaa tagataattc |
| 175861 | tttcattctt tggtgatgtt ggattagaga tgcbgtttta catgtgattc ttaatggaag |
| 175921 | ttcagtagtg taaagcttta aggtaattat atgtaaatat catgaattat tttgaaatgg |
| 175981 | ttggcaagtt aaaggcttaa catatcaagc aattaaatta ttaatcataa agattttttc |
| 176041 | taattgactt tattactgat aatttcttgc catctgtttt attttatttt tgctagtttc |
| 176101 | tgaaaatatt tcaataaaac tgctatctat gtaatttaaa gttattccta tgtttgcatg |
| 176161 | gaaacaattg tgatcatttt aattaaaaca catctttacc tcctaagaca aactaaccca |
| 176221 | catatcattc taattggcaa attaaaaaaa aaaattgtgg atatagatag agtaaaaaaa |
| 176281 | atcctgaaat attttatgcc atggctacac agcatcatag gcttttatat gataaacacc |
| 176341 | aaataatgaa aatagaaaca acccaatggc tatcagagat gtatcttaga agagtgtact |
| 176401 | tccctggttc tcttgaacca aaatgattat tctaagcaaa atgtttgaat tgtccaaaaa |
| 176461 | acaaactgtc aataggaagt gttattttaa agggttaata tgaatttttg tttcagtaat |
| 176521 | catagcatga ttaaaaacta taaattggtg gcaaataaag gagattgaaa gtcattttgt |
| 176581 | aaggtgaact ataaaactgt ggatatgtcc ttttcctcaa ctagaacttt aaaaagcagc |
| 176641 | ttttgcctta tttctactgt atctagtcat aaattatttt tctgagtaat actgaattgg |
| 176701 | attacaattt ttgttattgg tcaatatcta gtgaggggag aaacttcaac agttaattga |
| 176761 | acagagagaa tttaataagt gaattgttag gaggtataaa tgttaattag actgcagaaa |
| 176821 | aggtaaaaag agaacactaa agtatcatag tgacggtaac tactggaagc agctaccatc |
| 176881 | ccaagggctg gaggaacaat ggaaagggtt gaaacgaaaa cactgtagcg tagaggaaag |
| 176941 | accctaggag ctaaaactca ggcctctgca gaggggctga tgctctgtgg tgctggtttc |
| 177001 | tgtgggagag gtgatgaagc tggttctgct agtgttggta atattggtga taaggattca |
| 177061 | cttgcagtat ggcaaggacg ctcagctcca ggagtgaaga agagatgcta aggaggaagt |
| 177121 | gtcaccccac aaagcaaata tgaagcagag aggaagccac aggaaagacg gagaccctct |
| 177181 | tcagtatccc ctattgtcag agtatatatt gacccagcta acaaagcaga aatatgactc |
| 177241 | acaaaatacc agccccagca ccacaatatt aaagtgtagg agggtgtgtt tgaagttggg |
| 177301 | acaatagctt aaaaactgaa ataatctatt accaaaaagc tgatctctac aatgtggtat |
| 177361 | tgcatgcctc gatttaaaat tatgttgctc ttgtttagat tttgtttcct ttgagttctg |
| 177421 | gaccgcatag tgttttcgta atactaggaa cacagaaaag aaaagctaca tctcatgctc |
| 177481 | agtaggaact aaattccttt gaagatctct tcaaaggatt cacttccacc aaatatattt |
| 177541 | caaggtgaat tttaccaaat gaagttataa gtttaattaa cagataacat taagttacct |
| 177601 | ttgagaaata gatttttatt tttcccgatt tcaatttttc tttctttctt tctcttgaga |
| 177661 | cagggctaac aaaattttta agggcaaaga catcttcacc cctaatttat tatccttatt |
| 177721 | gagaaataaa atatttaaac gaaatgtaga attgagggaa ggtattcaag ggaacaaaga |
| 177781 | agagagggaa atatcttgat atatctgtaa ttattctttc cacagatttt ctggttcaga |
| 177841 | gagtttgagg ctctagacat aaaagaattg cttagggcgg gtgcggtggc tcacacctgt |
| 177901 | aatcccagca cattgggagg ccggggaggg tggatcacct gaggtcagaa gttcaagacc |
| 177961 | agcctggcca acgtggtgaa actctgtctc taataaaaat acaaaaatta gccgggcaag |
| 178021 | atggcagatg cctgtaatcc cagctactgg ggagcctgag gcaggagaat tgcttgaacc |
| 178081 | tgagaggtgg aggttgcagt gagccgagat tgtcccactg cactccagcc tgggcaacag |
| 178141 | aacgagactc tgtctcaaaa aaaaaaaaaa aaaaaaaaaa gcccggctta tgcctgtaat |
| 178201 | cccagcactt tgggaggccg aggtgggcag atcacctgag gtcaggagtt cgagaccagc |
| 178261 | ctggccaaga tggtgaaacc cccatctcta ttaaaaatat aaaaattagc tgggctaggt |
| 178321 | ggtgggcgcc tgtaatccca gctactcggg aggctgaggt agattgcttg aacctgggag |
| 178381 | gcagaggttg cagtgagcca agatcgcacc attgcactac agcctgggtg acaagagcaa |
| 178441 | aactccatct caaaaaaaaa aaaagaaaag aaaagaaaaa aacagaatgg cttagattca |
| 178501 | gattcaggaa ggcaaacact actgcttaaa gttataatag ttccagtaat cctgcatcag |
| 178561 | tgttcagatt tgagttctca tgcttatggg tcctaatgtt gactgcatat ggttacattt |
| 178621 | aaaaaaaatg agctaaatat ctgatacctg ccttccttaa gttccaaagt caagcaaagc |
| 178681 | ttaactttat taaggtgttt cttttttact tttaaagaga agaagaagaa attgatgaag |
| 178741 | aggaattgga aagattgaag gcagagttag atgagaaaag acaaataatt gctactgtca |
| 178801 | aatgcaaacc atggaagatg gagaagaaaa ttgaagttct caagtatggt gccacttttt |
| 178861 | ataagtagaa acactttccc tgatattgat tttctaaatc ctctgaatat tccatatata |
| 178921 | tatatatata tatatatata tatacacaca cacacacaca tacacacaca cacacacaca |
| 178981 | tacactgtat atatagtata tgtaatacta tatatatcat ttatatatga ctcattatat |
| 179041 | ataatggtca cagtcataaa catgtatatc atatttctgc atatataccc aaatatatgt |
| 179101 | atatttacat atatctgttt cttatcacta gagagactgg gctaatgatt ataaattgga |
| 179161 | agcaactctg agaaattatt tagagtcttt taaattcttt ggtcatcttt aatcatttgt |
| 179221 | tccttaaact ggagtttcct tagcttaata catttgttac catctctttg aaagagataa |
| 179281 | tatatttgaa ttttacaagt ttgtgtggat tgtgcactct tgtttctctt cagggccctg |
| 179341 | gtctcaaatg aacttttcat gtgacagctt acattaaagc acagagaaga tgctaggcca |
| 179401 | acagaatagt aaataggatt ccaattaaag gttatgctaa tagctattcc ccgaacatgt |
| 179461 | cagtgaccca atttcagtct tatgttgatg ctaatgtgga cgcgaacgca gtcagagcac |
| 179521 | acagatttat aaactcttca tatgaaaagt caaatgatta tcctattctg ggggatggga |
| 179581 | agattcaccc ctgaggatca cactatattt ccctgccctg gtggttggga gaggaggaaa |
| 179641 | gggcagggaa aagggaaact gtttgtgata acagccatgt ttgcatctcc tagttttgcc |
| 179701 | aaagattgca ttcttccata aatcattttt ataaatagta gtaagtttta aaaactatcc |
| 179761 | cttcacaagc ctatttaact agtaatgctg taaaaatgat agtatagtat tattgtctct |
| 179821 | gatgatgaaa acaagaactg aaatagctat caggataatt cacttctagt cttggttctg |
| 179881 | caagagcttg tgtgctattt aaaggtatcg cttaatctcc tgaagcctta gttacctcat |
| 179941 | atccaaaata aagaaagata ttctatatct tctctgtgcg cttgaagcgt agccatcatt |
| 180001 | tatagtattt ttgttttgtt ttgtagagta aggttttcca agttctaaac aatggttttc |
| 180061 | ccagaaaact tttgcaacac cacaaaattg cagtctggga actccctttg catgctcatg |
| 180121 | tacacatcca tcataaatga aggcaaaggc aagcagcatc ctacgttcca cagagctaca |
| 180181 | aattttgtgt atgcatgctc atgtgtctgt acattaaaat aattttcatt tagcgaagtg |
| 180241 | ataatgatgc taaattatag gttttaaaaa tattcttacc tggcaaaata gaagtttggc |
| 180301 | aaccctatgt gggcctctca aatatatttg aaatattaat gacccataaa gtccccaaac |
| 180361 | ttgggaactt cagataacta tatatagaaa gtttaaaatt ggagcaatat tgtgaacttc |
| 180421 | caaaatatat gattgaataa aaattccaag gactaccttt cctttccaac tgcatggagg |
| 180481 | tgtagaaata cccccctggt ctttgcccgt atctctcgtg caggatcgtg gaggagacag |
| 180541 | cagcttcaga tttgagttag gatctcagat cttccattta atagctgtgc acatttgatc |
| 180601 | gagttgtgta gtctctctac acaactcagg ttccttatga aattgtacct tattgacaga |
| 180661 | gtttttggaa ttataaaatg agataattta tgtaaaacat gttgccagaa cttggcacat |
| 180721 | agcbgggaaa cctaagttgt agttattact tcatctcaga acagacttga tgcccaattc |
| 180781 | ctgaagggtg acaagagaga tggagagaat ttgggacctg gcaagtctag gtgggagcag |
| 180841 | acctagtcac aatcagaccc cagaagcctt gtgggatggt gacacattgg taccagtttg |
| 180901 | gataaacaat aagattttga ggatgcaaca gaaggaagta aggcatttga caatttagca |
| 180961 | aaatccagga ccagaccgtt ctcagatctt ctaaaacata gccatagttc tgtggcagat |
| 181021 | ttcaaggtct caggccttgg tctggagtcc aagagttaag caaattttag caatggggag |
| 181081 | agataggcat tagcattgga aagaaggaca atacgagccc ttagtacgaa agcagacact |
| 181141 | gacaataagg agaccgcacc tgactctaat ctagacatag gtttttttat ttggcccaca |
| 181201 | tgtgttttgt ttctttttct tttatttttt ttaaccgtgg caatatatac aatacaaaat |
| 181261 | tcaccatttt aaccatgaaa tcgattttct ttatcataat ttttttcaat atttttaaaa |
| 181321 | atttaaatta cttttttttt ctcgagacag ggtcttaact ctgttgccca tgctggagtg |
| 181381 | cagtggtgct atcttagctc actgcagcct caatctcctg ggagcaaggg atccttctgc |
| 181441 | ctcagtctac cacgtacctg ggactatatg tgcacactac catgtttggc taattttttc |
| 181501 | tttttgtgta gggatggggt cttactatgt cgcccaggct gatctcgaac tcctggcctc |
| 181561 | aagccatctt cctacctcag cctcccaaag tgctgagatt ataggcatga gccactgcac |
| 181621 | ccagcctcaa tcttttaaaa gtgggagtaa aatattcaga tgttcagctt ttcatgaaca |
| 181681 | agtggaagat ttggtcctac attcttccat cacagtaaca gctaggacta agcacctggc |
| 181741 | ctctattaat ggggcattcc tttttcaatt tgcaccattc tcaaacaatc catattactt |
| 181801 | gtttatgctg cttggctgga ccctgtagac attcagattt gctgcctcct accagtgcag |
| 181861 | ctggggaaca agctaggcat gttaccaaga cggtgattca gatcttcaaa gccaaagtca |
| 181921 | gctcacagaa tggaagcaca aatttagtcc cagaaaagaa acaactgtgg cttctcacat |
| 181981 | gtcagcaagg ctcacagtct ggtgatgatt ttccatggcc agaagtggtt tctgccttgt |
| 182041 | ggccgcacct atgggttcca gctggtgcca tggccggggc cagcctaatt cttttggttt |
| 182101 | cagtctccgt gatgaagttg ttggtactta agtgcctgac ttatctctct ttgaatgctc |
| 182161 | tattgagctt tttttgaatg ttctattgag cttctcccca ccagattcca actacattat |
| 182221 | tagaacaagt ggtcagtact tcacagaacg tgcaggtaaa tgtttctcta ccccaatgcc |
| 182281 | accaatgctt gggagtgagg cagctttccc tctacccttt atttctttca gagacatcta |
| 182341 | actttgagag cgtgtttcta aggattatgt tattaatatt ccccaaatta gcctgcatca |
| 182401 | tcgctcctgg ctaagtctgt gaaacccaag atactaagct cagtgtaact gaaagagatg |
| 182461 | ttttgatcac agctttgaat ctgtgcctgg gttcgaagcc ttcttgttct ggacttggag |
| 182521 | aaagttttga aacttctaaa agatatttta gacagtgtca gggttttaag atatatttta |
| 182581 | gagtaagcct caggaatggc aacttatggt tctgagggtg ttatgggaga ctcagagata |
| 182641 | gttcccacat ttttaaagag catctaccat taggaacccg agcacactca gggcttagga |
| 182701 | tcaaaagatg gttaatataa gtggttgcta cataggcagg cagctctagg aattggttgt |
| 182761 | gaatccatat taatcctcaa acatggctca ggagctagag ttcagcattt tcaagatagc |
| 182821 | agaattggct ctatacagct gaaaggaagc catctccttt tcatcatcac tgtggaaatt |
| 182881 | gtgattttca ttctatgttt tattgtatat caatacaatt tcacacattc ctgtagcctt |
| 182941 | aaacctactg aatgataatc tcttggtgga aaggcaggtg tgtctggccc agaccagaaa |
| 183001 | tgtatatttt tattttttga tctaaaaatt actgatatat agtcagaatg aaagctgctg |
| 183061 | ttttcaattc tgagagacag aaagctccac taaacagaat actcagctct acagatgatt |
| 183121 | tatacaatga ggaagagtgt gtgtagggag ttggaggggg gtaagggaaa cagtgtcata |
| 183181 | ttttccaatt atttgaaagt caatttctat attttattaa tcacatcatt taaaaatatc |
| 183241 | cattaatgag aatactaagt ataatctgtt tttaagctgt tcatgggttt aattcagagg |
| 183301 | ctgtatgact agatctttcc tacctataag aggtcgcagc acttacctga tagcagccac |
| 183361 | cacaagtgaa ataaaatacc caggtagaaa gtaacaggtc tacttattac ttgtcttgaa |
| 183421 | acctaagact tttaactgta tcagaacatt atatgtggta gattgaaagt gctcatgact |
| 183481 | aaacctaaat aagcaaccac cttattttgt tacagtagaa taggttttag actgaaaatt |
| 183541 | gagacttgga ctttgagcta agtgcttcct ataacttctc tttgaacctc catttattca |
| 183601 | tttataaaac agataaattg gattgtggac catggtcttc aaactgctat cctccaaagt |
| 183661 | caagcagttt ggtgaaatca aatcagggca aaggaggcag aggtcgcagg gagagctact |
| 183721 | gggccacaat gccggcttca aaaacagacc tatggtttct ctcttctaca cagtgtactt |
| 183781 | ccatataaag cattgattgg aagaaagaat gcttctgtgg ctattaaaat aaaaaacagt |
| 183841 | tttaaaaacc cacttagctg gatatttaaa gaaacttatt ttggagaagg gacagttcta |
| 183901 | acatttcatg agtctacaaa ttaggataca gaaacactcc aagtatactt ttgcagggaa |
| 183961 | ttggcttttg gatagacacc attttaaaat gatttatttt gataacaaaa atgtttattt |
| 184021 | agctattata ctgactactt taatttctga gctttgtagt accctgacct attgtttctg |
| 184081 | ggagttgtcc attggctggc taggcctggt gatttattaa tgagacaccc agtaaagcca |
| 184141 | ggaaagttat cctcagattt cattcacact taaacaggtg cttcaaactg tcagctggcc |
| 184201 | attaaggtaa cattcctcct tactcaatct cccttgatct tttcacactt cttttcattt |
| 184261 | gtttgtttgt tttttatttt gttttatttt ttagttggga gaatgaatac atttttagga |
| 184321 | aagtgaaaac agaccatggt tgatttcctc taatggagct gttgggaaaa ctttgggaaa |
| 184381 | actatttatt tggcttccat tctcaccctt tctctcctgt tttcttgtag cttaagattt |
| 184441 | tcacaggcaa gaatcatggt ttatacttta gtttttctct catctgtcat tgtgaaggaa |
| 184501 | taaaagatgc tatacaagag gacatttaag cgtgtgaaca cacccaccaa cataccctca |
| 184561 | aaccattctg gattattttc ttttttaaat tttatatttc cataagtttt tggggaacag |
| 184621 | gtggtatttg gttaaatgag taagttcttg gggaacaggt ggtatttggt tacatgagta |
| 184681 | agttcttctg tgatgattta ttagattttg gtgcacccat cacctgagca gtatacactg |
| 184741 | aacccaattt gtagtctttt atccctcatc ccaatcccac cctttcccca gagtccccaa |
| 184801 | agaccactgt atcattctta tgcatttccg tcctcatagc ttagcttcca cttacgaatg |
| 184861 | agaacatatg atatttggtt ttccattcct gaattacttc acttagaata atagtctcca |
| 184921 | gttgtatcca ggttgcbgtg aatgccaata tttctttctt tttctttttc tttctttctt |
| 184981 | tttttttttt ttgagatggg atcttgctct gtcacccagg ctgtagtgca gtggcgtgat |
| 185041 | ctcggctcac tgcaaccttt acctgccagg ttcaagcgat tttcctgcct cagcctcctg |
| 185101 | agtagcbggg attacaggca tgtgccacaa cacccagcta atttttgtat ttttagtaga |
| 185161 | gatggggttt tgccatgttg gccaggctgg tcttgaactc ctgacttcgg gtgatctgcc |
| 185221 | caccttggcc tcccaaagtg ctgggattac aggcgtaagc caccatgcca ggcccaaatg |
| 185281 | ccaatatttc actccttttt atggctgagt agtattccat catgtatata taccacaatt |
| 185341 | tctttatcca ttcattgact gatggacatt tggtctggat tcatatttct gcaattgtga |
| 185401 | attctgctgc tataaacatg catgtgtaag tatccttttt gtttaatgac ttcttttcct |
| 185461 | ctgggtagat accctgtagt gggattgctg ggtcaaatgg tagttctact tttagttctt |
| 185521 | taaggaatct ccaccctgtt ttccatattg gttgtactag tttacattcc caccagcagt |
| 185581 | gtagaagtgt tcccttttca ccacatccat gccaacatct attatttttt gatgttttga |
| 185641 | ttatggccat tcttgcaggt gtaaggtggt attacattgt ggttttgatt tccattttcc |
| 185701 | tgatcattag tgatgttgag cattttttca taagtttttt ggtcgtttgt atatcttctt |
| 185761 | ttgaaaattg tctattcatg accttagccc actttttgat aggactgttt gtatttttct |
| 185821 | tgctaatttg tttgagttct ttgtagattc tggatattag tctttgtcag atgtagagat |
| 185881 | tgtgaagacc ttctccctct ctgtgggttg tctgtttact ctgctgactg ttccctttgc |
| 185941 | tgtgcagaag ctctttagtt taattaagtc ccacttattt atctttgttt ttgttgcatt |
| 186001 | tgctttgggg ttcttggtca tgaagtcttt gcctaagcca atgtctagaa ggttttttct |
| 186061 | gatgttatct tccagaattt ttatagtttc aagtcttaga tttaagtcct tgatccattt |
| 186121 | tgagttgatt tttgtataag gtgagagatg aagatccagt tttattctcc tgcatgtggc |
| 186181 | tagccaatta tcccagcaac atttgttgaa tgaatagggt gtcttttccc cacattatgt |
| 186241 | ttttgtttgc tttgtcaaag atcagttggc tgtaagtatt tggatttatt tctgggttct |
| 186301 | ctattctatt ccattggtct atgtgcctat ttttatacca gtagcctgct gttttggtga |
| 186361 | ccatagcctt atagtatagt ttgaaggaag gtaattggat gcttccagat ttgttctttt |
| 186421 | tgcttagtct tgatttggct atgtggcctc ttttttggct ccatatgaat tttagaattt |
| 186481 | tttttctagt tctatgaaga atgatggtgg tattttgatg agaattgcat tgaatttgta |
| 186541 | gattacttta ggtagtatgg tcattttcac aatagtgatt ctacccaacc acattagcat |
| 186601 | ggatgtattt ccatttgttt gtgtcatctg tgatttattt cagccatgtt ttgtagtttt |
| 186661 | ccttgtagaa gtatttcacc tccttggtta ggtataatcc caagtacttt tttttttttc |
| 186721 | ctgtggctat tgtaaaaggg gttgaatagt tgatttgatt ctcagcttgg tcactgttgg |
| 186781 | taaacagcag agctactgat ttgtgtacat taattttgta tcctgaaact ttgctgaatt |
| 186841 | catttattag ttccaggagc tttttggagg agtcttcagg gttttctggg tatatgatca |
| 186901 | catcatcagc aaacagtgac agtttcacct ctttaccaat ctggatgccc tttatttctt |
| 186961 | tctcgtgtct gattggtctg gctagtacta tgttgaatag tagtgagagt gggcatcctc |
| 187021 | gtcttgttcc agttctcaga gggaatgctt tcaactattc cctgttcagt attatgttgg |
| 187081 | ctgtatgttt gtcatagatg gcttttatta cattgaggta cgtcccttgt atgccgattt |
| 187141 | tgctgagggt tttaatcaaa atgggatgct ggtttttgtc aaatgctttt tctgtgtcta |
| 187201 | tttttttttc gggggggtgg gtgtttaaga gcagaaagtt taataggcaa aagaaagaag |
| 187261 | agaaaaccac ccccatgcag agggaggggg atcctctctt gctgcatcta ttgagatgat |
| 187321 | catgtcattt ctgttttaat tctgtttatg tgttgtgtca catttattta caagtgtatg |
| 187381 | tcaaactatc cctgcatcca tggtatgaaa ttcacttgat catagtggat tatctttttg |
| 187441 | atatggtgtt ggatttggtt agctagtatt ttgttaagga tttttgcatc tatgttcatc |
| 187501 | agggatattg gtctgtggtt ttcttttttt gttttgttct ttcctggttt tggtattagg |
| 187561 | gtgatactgg tttcatagaa tgatttaggg agtactccct ctttctcttt cttgtggaat |
| 187621 | agtgtcaata ggattgaatg tctgatagaa ttcagcbgtg aatctgtctg gtcctggact |
| 187681 | tttttttgtt ggtaattttt aaattactat ttcaatttca ttgcttgtta ttggtctgtt |
| 187741 | cagggtttct aattctgatt ctagctaaat cctcctgatt taagctagga ggattgtatc |
| 187801 | tttgcaggaa tttattcatc tcttctaggt tttctagttt atgtgcataa aggtgttcat |
| 187861 | agtagccttg agtgatcttt tgtatttctg tggtgttggc tgtaatatct cctatttcat |
| 187921 | gtctaataga gcttatttgg atcttctctc ttctttgctt cattaatctt gctaatcgtc |
| 187981 | tatcaatttt atttatcttt tcgaagaacc agctttttgt tcaatttatc ttttgtattt |
| 188041 | ttgttgttgt ttcaatttca tttagttctg ctctgatctt tgttatttct tttcttctac |
| 188101 | tatttgggtt tggtttgttt ttctagttcc ttgaggtgtt accttaggtt gtctatttgt |
| 188161 | gctctttcag actttttggt gtaggcattt aatgctatga acttttagca ccacctttga |
| 188221 | tgtatcccag cagttttgat aggttgtgtc actattattg ttcagttcaa agaatttttt |
| 188281 | catttccatc ttgatttcat tgttgaccca acaatcattc aggagcatgt actttaattt |
| 188341 | ccatgtattt gcatggattg gaggatttct tttgatttcc aattttattc cactgtggtc |
| 188401 | taagagagta cttgatacaa tttcagtttt cttaaattta ttgaaacttg ttttctggcc |
| 188461 | tgtcatatgg tttatcttgg agaaagttcc atgcactgat gaatggaatg tatactctgt |
| 188521 | ggttgttggg tagaatattc tgtaaatatc tgttgggtcc atttgttcta ggtatagttt |
| 188581 | taatccattg tttctttgtt gactttctgt cttgatgacc tgtctagtgc tgtcagtgga |
| 188641 | gtattgaagt cccccactat tattgtgttg ctgtctatct catttctgag gtctagtagt |
| 188701 | aattgtttta taaatttggg agctccagtg ttaggtgcat atttatttag gattatgttt |
| 188761 | tcctgttgaa caaagccttt tatcatgatt tagtgtccct ctttgtattt tttaactgct |
| 188821 | gttgctttaa agtttgtttt gtctgatata agaatagcta ctcctggtca cttttggtgt |
| 188881 | ccatttgcat ggaatgtaat tttccaccct tttacctcaa ctttatgtga gtccttatgt |
| 188941 | gttagttgag tctcttgaag acagcagata cttagttggt gaatactttt tttttaaatt |
| 189001 | atactttaca ttctggggta catgtgtgga atgtgcaggt ttgttacata ggtatacaca |
| 189061 | tgccatggtg gtttgctgca cctgtcaacc cgtcatccac attgggtatt tctcctaatg |
| 189121 | ctgtccctcc cttagctccg catccccaaa caggccacag tgtgtgtaca tgtgttctca |
| 189181 | ttgttcaact cccacttatg agtgagaaca tgtggtgttt agttttctgt tcttgtgtta |
| 189241 | gtttgctgag aatgatggtt ttcagcttca tccgtgtccc tgcaaaagac attaactcat |
| 189301 | ccttttttat ggctgcatag tattccatgg tgtatatgtg ccacattttc tttatctagt |
| 189361 | ctatcattga tgggcatttg ggttggttcc aagtctttgc tattatgaat agtgctgcag |
| 189421 | taaacatacg tgtgcatgtg tctttacagt aaaatgattt ataatccttt gggtatatac |
| 189481 | ccagtaatgg gatggctggg tcaaatggta tttctagttt cagatccttg aggaattgcc |
| 189541 | acactgtctt ccacaatggt tgaactgctt tagactccca ccaacagtgt aaaagcattc |
| 189601 | ctatttctcc acatcctctc cagcacctgt tgtttcctga ctttttaatg atcaccattc |
| 189661 | taactggcgt gagatggtat ctcattgtgg ttttgatttg catttctcta ataaccagtg |
| 189721 | atgatgagcc tttttttcat atgttggttg gctgaataaa tgtcttcttt tgagaaatgt |
| 189781 | ctgttcatgt cctttgccca ctttttgatg gggttgtttg atatttcttg taaatttgtt |
| 189841 | taagttcttt gtagattctg gatattagcc ctttgtcaga tggatagatt gcaaatattt |
| 189901 | tctcccattc tgtagattgc ctgtccaatc tgataatagt ttcttttcct gtgcagaagc |
| 189961 | tcttcagtgt aattagatcc catttgtaaa ttttggcttt tgttgccatt gcttttggtg |
| 190021 | ttttagtcat gaagtctttg cccatgccta tgtcctgaat ggtattgcct aggttttctt |
| 190081 | ctagggtttt tacggtttta ggttttaggt ttaagtcttt aatacatctt gagttaattt |
| 190141 | ttgtataagg tgtaaggaag gaatccagtt tcagctttct gcatatggct agccagtttt |
| 190201 | cccaacacca tttattgaat agggaatcct ttccccattg tttgtttttg tcaggtttgt |
| 190261 | caaagatcag atggttgtag atgtatggcg ttatttctga ggcctctgtt ctgttccatt |
| 190321 | ggcctatata tctgttttgg taccagtacc atgcbgtttt tgctactgta gccttgtagt |
| 190381 | ataagtttga atcaggtagt gtgatgcctc cagctttgtt ctttatgctt aggattgtct |
| 190441 | tggctatgca ggatattttt tggttccata tgaaatgtaa agtagttttt ttcaagtctg |
| 190501 | tgaagaaagt caatggtagt ttgatgggga tagcattgaa cctacaaatt actttgggca |
| 190561 | gtatagccat tttcacgata ttgattcttc ctatccatga acatggaatg tttttccatg |
| 190621 | tttgtgtcct ctcttatttc cttgagcagt ggtttgtagt tctccttgaa gaggtccttc |
| 190681 | acatcccttg taagttagat tcctaggtat tttattcact ttgtagcaat tgtgaatggg |
| 190741 | agttcactca tgatttggct ctctgtttat ctgtttttga tgtataggaa tacttgtgat |
| 190801 | ttttgtacat tgattttgta tcctgagact ttgttgaagt tgctaatcag cttaaggaga |
| 190861 | ttttgggctg agacaatggg gttttctaaa tatataatca tgtcatctgc aaacagagac |
| 190921 | aatgtgactt cctcttttcc taactgaata ccctttattc ctttctcttg cctgattgcc |
| 190981 | ctggccagaa cttccaatac tatgttgaat aggagtggtg agagaagaca tccttgtctt |
| 191041 | atgcbggttt tcaaagggaa tgcttatgct tccagttttt gcccattcag tatgatattg |
| 191101 | gctgtggatt tgtcataaat agctcttatt attttgagat acattccttc aatacctagt |
| 191161 | ttattgagag tttttagcat gaagggctgt tgaattttgt tgaaggcctt ttctgcatct |
| 191221 | attgagataa tcatgtggtt tttgtgattg gttctgttta tgtgatggat tacatttatt |
| 191281 | gattcgcata tgttgaacca gccttgcatc cccaggatga agccagcttg atcatgttgg |
| 191341 | ataagctttt tgatatgcta ctggattcag tttgccagta ttttattgaa gatctttgca |
| 191401 | ttgatgttca tcagggatat tggcctgaaa ttttcttttt ttgttgtgtc tctgccaggt |
| 191461 | tttgctatta ggatgatgct ggcctcataa aataagttaa ggaggattcc ctctttttat |
| 191521 | attgtttgga atagtttcag aaggaatgat accagatcct tttagtactt ctggtggaat |
| 191581 | tcatctgtga atttgtctgg tcctggactt tttggttggt aggctattaa ttgctgcctc |
| 191641 | aatttcagaa cttgttattg gtctagtcag ggattcaact tcttcctggt tcagttttag |
| 191701 | gagggtgtat gtggctagaa acttatccat ttcttctaga ttttctagtt tatttgcata |
| 191761 | gaggtattta tagtattctc tgatggtagt ttgtatttct gtgggatcgg cggtgatata |
| 191821 | ccctttatca ttttttattg catctacttg atttttctct cttttcttct ttattagtct |
| 191881 | ggctagttgt tgatctttca aaaaagccag ctcctggatt cattgatttt tttttttttt |
| 191941 | gaagggcttt ttgtatctct gtctccttca gttctgctct gctcttagtt acttcttgcc |
| 192001 | ttctgttagc ttttaaattt ttttgctctt gcttctctag ttcttttaat tgtgatttta |
| 192061 | gggtgtcgat tttagatctt tctttctttc tcttgtgggc ttttagtgct ataaatttcc |
| 192121 | ctcaacatac tgctttcaat gtgtcccaga gattctggta cattgtttct ttgttctcat |
| 192181 | tggcttcaaa gaacatcttt atttgtgcct taatttcatt atttacccag tagtcattta |
| 192241 | ggaacaggtt gttcagtttc gatgtagttg tgaagttttg agtgagtttc ttaatcctga |
| 192301 | gttctaattt gattgcactg tggtctgaga gactgttatg atttccattc ttttgcattt |
| 192361 | gctgaggagt gttttacttc caattacgtg gtcaatttta gaataagtgc gatgtggtgc |
| 192421 | tgagaagagt gtatattctg tttatttcgg gtggagagtt ctttagatat ccattaggtc |
| 192481 | tgcttggccc agagctaagt ttaagtcctg gatatccttg ttaattttct gtctcgttga |
| 192541 | tctgtctaat attgacagtg gggtgttaaa gtctcatgct attattgtgt gagggtctaa |
| 192601 | gcctctttgt aggtctctaa ggacttgctt tatgaatctg ggtgctcctg tattgggtgc |
| 192661 | atatatattt aggatagtta gctcttcttg ttgcattgat ccctttacca ttatgtaatg |
| 192721 | ctcttctttg tctttttttg atgtttgttg gtttaaagtc tgttttgtcg gagactagga |
| 192781 | ttggaacctc tgcttttttt tgcbgtccat ttgcttggta aatattcctc catcccttta |
| 192841 | ttttgagcct atgtgtgtct ttgcacgtca gatgggtctc ctgaatacag cacaccaatg |
| 192901 | ggtcttgact cttaatccaa tttgccagtc tgtgtctttt aattggggca tttggcccat |
| 192961 | ttacatttaa ggttaatatt gttatgtgtg atttgatcct gccattatga tgctggatgg |
| 193021 | ttattttgct cattagttga tgcgatttct tcatagcgtt gatggtcttt acaatttagt |
| 193081 | atgtttttgc agcggcbggt actagttgtt cctttccatg tttagtgctt ccttcatgag |
| 193141 | ctcttgtaag gcaggcctgg tggtgacaaa atctctcagc atttccttgt ctgtaaagga |
| 193201 | ttttatttct ccttcactta tgaagcttag tttggctgga tatgaaattc tgggttgaaa |
| 193261 | attattttct ttaagaatgt tgaatattgg cactgtcttc tggcttgtag ggtttctgca |
| 193321 | gagagatcta ctgttagtct gatgggcttc cctttgtggg taacccaacc tttctctctg |
| 193381 | gctgccctta atattttttc tttcatttca accttggtaa atctgatgat tacatgtatt |
| 193441 | ggggttgctc ttcttgagca gtatctttgt ggtgttctct gtatttcctg aatttgaatg |
| 193501 | ttggcttgcc ttgctagctt ggggaagttc tcctggataa tatcctgaag aatgttttcc |
| 193561 | aacttggttc cattctcccc atcactttca ggtacaccaa tcaaacatag atttggtctt |
| 193621 | ttcacatgat cccatatttc ctggaggttt tgttcatttc ttttcactgt tttttatcta |
| 193681 | atcttgtctt cttgctttat atcattgaat tgatctccaa tctctgatag cctttcttct |
| 193741 | gcttgatcga ttcggctatt catactcgtg tatgtttcat gaagttctcg tgctgtgttt |
| 193801 | ttcagctcca tcaggtcatt tatgtcctct aaactggtta tttcagttag caatttgtct |
| 193861 | aacctttttt caaggttctt agcttctttt cattgggtta gaacatggtc ctttagctcg |
| 193921 | gaggagtttg ttattaccca ccttctaaag cctacttctg tcaattcgtc aaactcattc |
| 193981 | tccatccagt tttgttcgct tgctggcaag gagttgtgat cctttggagg agaagaggca |
| 194041 | ttctggtttt tggaatgttc agccttttcg cactggtttc tccccatctt cgtggattta |
| 194101 | tctacctttg gtctttgaag ttggtgacct tcggcbgggg tctctgagtg gtcctttttg |
| 194161 | ttgatgttga tactattcct ttctgtttgt tagttttcct tctaaccatc aggcccctct |
| 194221 | gctgcaggtc tgctggagtt tgctggaggt ccactccaga ccctgttcac ttgggtatca |
| 194281 | ccggcagagg ctgcagaaca gcaaagattg ctacctgttc cttcctctga aagctttgtc |
| 194341 | ccagagggac atccaccaga taccagccag agctctcctg tatgaggtgt ctgtcagccc |
| 194401 | ctactgggag gtgtttccca gtcagtatac ctgggggtga gggactcact tgaggagtca |
| 194461 | ctgtgtccct tatcagagct ggaacccagt gttgggaaat ctgctgctct cttcagagct |
| 194521 | gccacacagg gcbgtttaag tctgctgagg ttgcacccac aaactcccct tcccccaggt |
| 194581 | gctctgtccc aggaaggtgg gggttttacc tatgagtccc tgactggggc tgctggcttt |
| 194641 | ttttcagaga tgccctggcc acagaggagg gaatctagag aggcagtttg ggtgcagtgt |
| 194701 | ccttactgag ctgtggtgag cttcgtccag tttgaacttc ctggcaactt tgtgtacact |
| 194761 | gtgagggtaa aaccccctac tcaagcctca gcaatggcag acgtccctcc ccccaccaag |
| 194821 | ctcgagcatg ccagatcgag ctcagattgc tgtgctggca gcgagaattt caagccagtg |
| 194881 | gatcttagct tgctgggctc cgtgggagtg ggacccactg agccagacca cttggctccc |
| 194941 | tggcttcagc cccctttcca ggggcgtgaa tggttctgtc tcactgacat tccaggcacc |
| 195001 | actggggtat gaaaaaacaa ctcctgcggc tagctctgtg tctgcccaaa cggcagccca |
| 195061 | gttttgtgtt ggaaacccag caccagggcc ctggtggcgt gggaactgga gggaatctcc |
| 195121 | tggtctgcag gttgtgaaga ccatgggaaa agtgcagtat ctgggccggt gtatgtggta |
| 195181 | cagtccatag tggcttccct tggctaggag agggagttcc ctgatccctt gcacttcctg |
| 195241 | ggtgaggcaa cgccccaccc tactttggct tgccctcctt gggctgcatc cactgtccaa |
| 195301 | ccagtcccaa tgaaatgaac tgggtacctc agttggaaat gcagaaatca cccaccttct |
| 195361 | gcatcgatct tgctgagagc tgcagactgg agcbgttctt gttttgccat cttgccagca |
| 195421 | aatcggtgaa ttgttatgta ttctgcaatt ctatatcttt taagtggagc atttaggcca |
| 195481 | tctgcattca atgttagtat tgagatgtga ggtactattc tattcatcat actatctgtt |
| 195541 | gcctgaatac cttggttttt tatttattta cttatttatt gtatttttgt tttaatgggt |
| 195601 | cttgtgagac tcatgattta gagattctgt tttgatgtgt ttccatggtt cgtttcaata |
| 195661 | tttagagctc ctttcagcag ttcttatagt gctggcttgg tagtggcaaa ttctcttggc |
| 195721 | ctttgtctga aaaagactgt atctttcctt catttatgaa gcttaggata caaaactgtt |
| 195781 | ggctgataat tgttttgttt aaggaggata aaaataggac cccagtcccc tctagcctgt |
| 195841 | agggtttctg ctgagaaatc tgctgttact ctgatagatt ttcctttata ggctacctgg |
| 195901 | tgcttttgcc tcacagctct taagattttt ccctctgtct tgactttaga taacctgata |
| 195961 | actataggtt taggcaatga tctttttgca atgaatttcc caggtgttcg ttgaacttct |
| 196021 | tgtatttgga tgtctacatc tccagcaagg ctgaggaagt tttcctctat tattccccca |
| 196081 | aatatgtttt ccaaacttag atttctcttc ttccttaggg actccactta ttcttaggtt |
| 196141 | tggtcattta acataatccc aaacttcttg gaggctttat tcatttttta aaattctttt |
| 196201 | actttccagt acattttgca tttctctgtg ttctttactt cctgaagtag tgattgtttt |
| 196261 | atatttatgc tatctatttc actgaagatt tctcccctca tatcttgtat catatttttt |
| 196321 | tatttcctta agttggactt cacctttttc tggtgcctcc ttgattagct tactactcga |
| 196381 | ccttctgaat ttttttttct ggcaattcag ggatttcata ttggtttgga tccattgctg |
| 196441 | gtgagctatt gtgatttttt gatgatgtta aagaacatta ttttgtcata ttaccagaac |
| 196501 | tgtgtttctg gctccttctt atttgggtag gctatttcag aggaaagatc tgaagctcaa |
| 196561 | ggctgctgtt cagattcttt tgtcccatgg ggtgttccct tgatgtagta ctctccccct |
| 196621 | tttcttagga atgtggcttc ctgagagcca aactgtagtg attgttattt gtcttttgga |
| 196681 | tctagccacc cagtggagct actggtctct gggctggcac tgggggatgt ctgcacagag |
| 196741 | tcctgtgatg tgaaccatct ttaggtctct cagctgtgga taccagcatc tggtccagtg |
| 196801 | gaggtggcag aggagtaaaa cagactctgt gaggtgcctt agttgtagtt gtttaatgca |
| 196861 | ctagttttgt gctagttggc ctcctgccag gaggtggcgc tttcaagagt acgtcagctg |
| 196921 | tggtagtata ggggaggatc aggtggtggg tgggggccct agaactccca agaaaatatg |
| 196981 | aactttgtct ttagctacca gggtgggtag ggaaggacca tcaggtggga gcagggttag |
| 197041 | gtacgaatga cctcagactc tccttgtgtg gggcttgctg tggctgctgt ggggataggg |
| 197101 | gtgtggttct caggtcaatg gagttatgtt cccaggagga ttatggctgc ctctgctgtg |
| 197161 | tcatgcaggt tgtcaggaaa gtggagaaag ctgatagcct cacccagctc ccatgcaacc |
| 197221 | caaaaggctg gtctcattcc cactgtgccc cctccaacag cactgagatt gttttcaggc |
| 197281 | agtgggcaag tagggctgag aacttgcccc aggttaccag cctcctggtt gagaaagcaa |
| 197341 | gcggggcttt tgtgccacct ccctgcctgt caccggattc acatcctccc ccaagttctg |
| 197401 | gccaggaaac ttcacatttg gttggaatcg ttacagagtt cagctggagg tttccttctc |
| 197461 | cctgtggtct tttcccagtt cctctggcag ccctcctcaa ggactcctgt gagacaagtc |
| 197521 | agaaattgct tctctgggga cccggagaac ccacatggct tttcctgtgg cttcttctac |
| 197581 | ccttgtgttt cactcagctc tctaaattgt ctcagctcca ggtaaggtca aatctttctc |
| 197641 | ttgtgatctg gacctttagg ttccccagtg agtgtgtata ttcggggcag gggatgatct |
| 197701 | ccctttccca cttttgcagt ttgggcactc acagtatttg ggctgtctcc caagtcctgc |
| 197761 | aggagcaacc tgtttccttc aaatggtctg tggattctct tgactttccc agtatatttc |
| 197821 | tgcagcagtt cttggaacaa aagttcacaa tgcaagtctc cacattctat tctatccatc |
| 197881 | caagtggggg ctgcaattta gtcctgactc ctatctgcca ttttttccta gcagttcatt |
| 197941 | ctggattatt tttaaccaat aactcaacca aagcacaggc cttcagttct tgtactttac |
| 198001 | aggtatcttg agtatagcaa tttttcatct gcagaatgga gagtatatag gcaattaatg |
| 198061 | catctagatt ttgtccatca aaaggtaaca ttgacatccc atttttctgc tattttataa |
| 198121 | gttcatacca ggtgtcaagc attatgctgt gggcaagttt cttagtctct acatccttga |
| 198181 | gtctccttac tatgaatgga gataaaagta ctactgcata aagttattga gataattaaa |
| 198241 | taagctcata gatacaagca tgtaacatgg tacctagcat aataattttc tttatccctc |
| 198301 | ttaaccctcc ttcaaggtag gttttactat tttgacttta catataagga aactgaaact |
| 198361 | ctgagaggat aaataattgt cagacaatta ataagtgata gatccacaat ttaagcttgt |
| 198421 | tgtgtggaaa cttaccttgg tatgatggag gtctctaagt acactaccaa gcaagctctc |
| 198481 | ccagaagtca caaacctgaa gttcacatac caaattaaaa tgctcaaatc acatggctta |
| 198541 | gtaagcttat ttaaagcaca gtgagaagta aggggaaaga gatgggttat ggtaagacat |
| 198601 | gggataaagt agcaatatcg tgagatcatg ggcaacagga ccacacttct gtatcctgtg |
| 198661 | ttctgcagtg ttcgtgtgcc ctgtctctct catcctgctg atagtgtggc taaggggact |
| 198721 | gaggtcccaa tgggggccac ataataaaga ttacttgggc caatgatagg tacaagctcc |
| 198781 | attggaatga atgacacagg acaagtacgc ctgatcatag cagtagcttg tgattagcct |
| 198841 | gctgaaattg caggagttgt gtctgcattt cttggacaga gcagatgcaa gaggaacaaa |
| 198901 | ctggatgaca ccaatggctg gtcaagttag cagtgacagg tatctcagtc ccaaagtgtc |
| 198961 | aggataatga tttggtcttt tcatctcttt ctaactgtaa tgttcttgtt tgttcatcat |
| 199021 | atatattttt aaaaatttat tttctgtctt tctttggcag atattacagg ttactaattt |
| 199081 | acctgctatc tgtagtgaac acatcaaaca tgcctatgca tcatatgtac atgtttactt |
| 199141 | attagctata tttaacatat cctatgtgca ggaccagcta cataatttgc atgatgtaat |
| 199201 | gtaaaatgca aatggggggc cccttgttaa aaaatactta agaatttcta ttattaagaa |
| 199261 | ttttaagatg atgaaagaaa ccagaatatg ccagctctaa atatgcctca ttgacgtaaa |
| 199321 | tatttttgag ctgaagggaa ttaagaagca catgcagaaa agctctctgc ctcttctatt |
| 199381 | tgcctaaaag caggacagag aattacaaaa acaaaaggtc ttttatgttc ctcctcccct |
| 199441 | ttcctgccta aagacaagat gtaaattctc ctttacaacc tttaccagct gagagcctgc |
| 199501 | ttcagaaaaa tctgggaaca gacttgactc tttcccacag ttctctggcc tttggaagct |
| 199561 | tagggcbgtt ttcctctttg tcctgtcact tctctacaat gtattgatct tgttgaatac |
| 199621 | tttgtattag ctgggattct aagccacact gccttgagtt acttttcacc tgcgtgatgt |
| 199681 | tcatgtatta atagacttgt ttgtttttct cttgttaatc tgtcatttgt tacaggagac |
| 199741 | tgtctcaatt aaaaacttac gagggttcag aaaaattata atttcttccc aacaatgaca |
| 199801 | atagcagagc ataaaactaa gcacagggcc catgtgagta tggggtcctg taacagtgca |
| 199861 | ggtcacaggc ctatgaaatt gcccctgcct atgaatttca cctgactatt ccaagccaag |
| 199921 | agctacttat tatctatgct gaacatttct aatgatttcc atcttttcca atagcagaat |
| 199981 | tcagtggtct caaataattc agccaactca cattatagaa ctcaagtcta tgtaactcca |
| 200041 | agtcactgtt catgcactat agtcatggag ttctttttaa atacctgtca tccccatgtt |
| 200101 | caaacccttc aatgtctccc tagtaattaa caaaatttag tcaattttgc ctaaaaaata |
| 200161 | tcggtgagct ccacaacctg aattccaact tactttccaa agcaaactct cattaattac |
| 200221 | tgatgatgac catgaggttt tggagaggga caaaaccatc tccaaatgtt tttgagtgtc |
| 200281 | tcctgtgtat cgtgtcctgt actagatgtt ttacacatca actcatttaa tccttatgag |
| 200341 | aacattgaaa acaaagttat ccccatttta cagatgagaa aattgagatt gatagaggta |
| 200401 | aggtcacctt acacagttat taactgtgag ggtggattaa aatccagatt tctctgtctc |
| 200461 | taaaacctca ggctttttct actgtgccat actacttcta atttgaactg tattttccag |
| 200521 | tgaaatgaga ttaatcttcc ctatagacat gccaggtacc tgttatcact ttattctggt |
| 200581 | atccgctcca ttttttggca tcctatgctt cttccatgcc tgactttatc tcccgtttga |
| 200641 | gcctctttct ctgccaacgc ccatagaaat aaaattcaaa tgaattctct tgattattca |
| 200701 | tttggctctt ctgttaggca gcattgtgtt gcatttttaa aaattatatg taaatctcca |
| 200761 | tttcagaaga gattttgagc tcctcagggc acgaaacgta tcacatagct ctttattgtc |
| 200821 | tcaagtacta gcatagtgct ttgtatgcaa tatttaatta atattttctg tgtgggtgaa |
| 200881 | aatgttaatg tcttttttgt tggacattca tttccaaagc agaacaatta acatgactgg |
| 200941 | atgtttaaag ggtctggatg aataggtgag tgaaggacag aaatgtgctg gtaaaaagcc |
| 201001 | ctgatctata gtgcttgcca atttccgtgg tgtaaatact tccactatag ccaattttaa |
| 201061 | gctaccaaca gttagagcca actcacaaaa ttcttgaaaa tttcataatc agtggttttg |
| 201121 | tgtgagccag cataagtcag cttcggtatc tctttgaggg atttaataat gaactcaact |
| 201181 | aaggtaaaac tgatctggaa caactgaaag cagaggttaa agtaaaataa ctctgttatt |
| 201241 | taatctataa tcaactatga aatgcttgaa ttagagctgc ctgtgagaag gaaaaacaat |
| 201301 | gattccataa tgtgaaggct ccagacaaag gccaaaagtg catcaatgac ttcttatccc |
| 201361 | agttttttaa actaaaaaaa ctgctagcta aatgattttt gttttatatg acttttgttt |
| 201421 | tatatgactt ttgtttatat gactttcctc ccaaatctgt tagcatgcat agagtttcag |
| 201481 | tttttctttc atcttctcct cttctgctca tttctctctt tttaaatttt atcataaagt |
| 201541 | ggaacacttt ttattcaaga gaaatcaatt cttgattgag atcagaaatc tcttgccatt |
| 201601 | cacattagag ttgatctacc acaattgcct gcttcatcat tttcattcta ttggaataag |
| 201661 | tatttacagc tataaactat cctgcatctt acatatttct attaaacaag aacaatgtcc |
| 201721 | gtgacagttt tctgtctcag gatttatctt attaatattt attgtattgt tatgagtgct |
| 201781 | tagcaaatta ttgtggttaa ctttaccaat gcctttcaaa taatattttg aaattttccc |
| 201841 | tgaaaggata ttgagaaaag ccaaatgtaa aaattattgt tttttttcct tgggatttgc |
| 201901 | atgttttgaa aacgttctca attgttaggg aagtcagctg taaaacacag gctaaatata |
| 201961 | cttttgtcta ccaagaattc cctttacttg tctgtttcgt gatttgttta atctttctta |
| 202021 | gttatgcatg taccatttta ccatagttac ctggagatat tcttgtctct gccttagttt |
| 202081 | ggcaggcttt tcaagtggct cactgagcta ctgtttctat ttcttttaga aagttgtgga |
| 202141 | ggcatttatt actctcataa ctgcctcaaa atctcaagca catggcctag ttaaatcatc |
| 202201 | ttgaggttct gcccatatgg ttgcagaagg atattagtca ttggaaaaaa tgggtctaaa |
| 202261 | atgataatat tcaaagatgt tttccaatat tcttggcaat cttattcact aatttccacg |
| 202321 | gacatttaat tagcaagaat ttaaagaacc ttgttttagt ttcctgctgc tgtaatagaa |
| 202381 | tatcacagac tggataatat ataaagaaag gaaatttatt gggcttatgg ttctggaggc |
| 202441 | tgggaagtcc aagaggcatg acactggcac ctggcaaggg tcatccaatg gtggaaggca |
| 202501 | tcacatggtg agcaaggacg agagacaggg agaaatgggg ctgtatttat ctttttatca |
| 202561 | ggagcctact ctgtgatact agcccactct catggtaacc acattaatac attcatgagg |
| 202621 | atagagccct catggccgaa ttatctctta aaggtcccat ctcttactac tgttacaatg |
| 202681 | gcaattaagt ttccaataca taaactttag gagacatagt caaaccatag aaatcttaaa |
| 202741 | cttcaaataa acagggattt tttttagtag cacagtataa aatgataata ataaatatca |
| 202801 | gatataaaaa atttctgaag ttctttttag atagataaat catggttact attatcttct |
| 202861 | tagttacatt tcattgcacc ctattttctg tggtattatc agtcaacata ttatatatag |
| 202921 | taagacacat taaattttct gagcttgcca gggcatcctg tcccctaaca gtttttgata |
| 202981 | atgatttatc aaatgtgtac tctttagaat tttagcbgtc agtaacagtt gattttcaca |
| 203041 | acttttatta gactatccat gatgtttttt ggaaaggtag ttaaaaggat aaatacatta |
| 203101 | attatcataa aattatctag gaatgctttt aaatacattt gacataatac attaacacta |
| 203161 | aaaggaaagc aaatatgcac ctaataagca caaaataaga ttaatcagag aaatgtgtcc |
| 203221 | ttgctgtttt ggggaagagt tatccagatc tcaggtaagg tactttttgt ggagataacc |
| 203281 | ttctgtttta tgaagcttgt aacaagataa caatcaaggc atttttgtgg ccatgtttat |
| 203341 | gtatatgagc aagcaatggc aagtgagata ctagtttggg ggcttaaata agataaaagt |
| 203401 | agttaaactt tattaaaatg cacatattaa aataaatttt gaaatacatt gtattcaaat |
| 203461 | attgtattag ggttctctta gacaactcat aggatacccc cacacacaca ttttatgtgt |
| 203521 | gtatgtatat atatatatat atatatatat atatatatat atatatatgt atatacacac |
| 203581 | acacatgcgt acatatatat acaggagttt attaagtatt aacttacatg atcacaaggt |
| 203641 | tccacaacgg ctgtctgcaa gctgaggagc aaggagagcc agtcccaaaa tcaaaactga |
| 203701 | agaacttgga gtccgatatt cgagggcatg aagcatccag cacaggagaa agatgttgtc |
| 203761 | ggggaggcta ggccagtctc tcattttcag atttttctgc ttggtttata gtcactggca |
| 203821 | gctgattaga ttgtgcccac cagattaagg gtgcatctgc cttccccagc ccactgactc |
| 203881 | aaatgttaat ctctctctct ttttttttga gacggagtct ggctctgtcg cccaggctgg |
| 203941 | agtgcagtgg cacgatctcg gctaactgca agctctgcct cccgggttca agccattctc |
| 204001 | ctgcctcagc ctcccgagta cctgggacta caggtggccg ccaccatgcc cggctaattt |
| 204061 | tttgtatttt tagtagagac agggtttcac tgtgttagcc aggatggtct cgatctcctg |
| 204121 | acctcgtgat ccacccgcct cagcctccca aagtgctggg attacaggcg tgagccacca |
| 204181 | cgcccggcct caaatgttaa tctcttttgg cagtacccac acagacatgc ctaggattaa |
| 204241 | ttctttgtac ccctcaatcc aatcaagttg acactcagta ttaaccatca caaatatcat |
| 204301 | aacaaatcgt gtgaaaggag gaaaggggaa tagttttaca tttcaaatat aaggggtcca |
| 204361 | agattccctt ttatagttat aaaaattaga aatttttaaa tggtatgaat cacttatttc |
| 204421 | aaatagttgt atttatgatg cttttgtaat atatatgcat aagtttacct atatatttat |
| 204481 | atatgtgtgc atgtacatat aatgtataaa tacacaaagg cactcatatc tacataatga |
| 204541 | tacatgattc tttggatgaa ttaactttta cattttatgc atacactctt attgtgtttc |
| 204601 | ttgaccagtg cttggtcaca gggatcagga caagttagaa gggaggctat gggaggacac |
| 204661 | tgtaaaatat taacacaact taattctgca gaaaggccag ttcacatagg aagatggtgt |
| 204721 | cctctgaggg tgcacttacc caagctgtgt tccccaattg cttaggacag tcttccatgt |
| 204781 | ctaagtttgt catgtggcaa acaatcaaat ttacctggta gtcctagtga tttgcagagc |
| 204841 | tcactgaaat gccatcgttt aaaagcatca ttttaaattt attgtccact gatagatcaa |
| 204901 | aagatagaat accctattcc aagagaaaaa ctaaagactt ctattagaaa aatttaggtt |
| 204961 | tagcacttct aaatatcact atacaacatt ttgaaaaatg ttttgaaaaa acagttataa |
| 205021 | tgcttagaaa catttagaag aatataagca cacaggtatt gtgttgaaac acattaccta |
| 205081 | cactttcaat gcaggatgct tcttctcact atggggcagc actttgtgaa tttttctgtt |
| 205141 | tcacagtatt cagaattcat tagaacatgt ttgattagaa tgcacatgag gcaagcaaaa |
| 205201 | ttagcacagt ctgtctatca agccacgtag atggccttgc aaagagctga atgctagtat |
| 205261 | atctaagaat atgagattat atgtatgatt catggtggaa atgtctcctt tgttagataa |
| 205321 | attcctgcaa accaatgcct ggaatgccct ctctgcctac tcgaattcta ttagctcttt |
| 205381 | aacatccatt tgaattctta tctcatctac aatatcttca gcaactctga agcctccatt |
| 205441 | gtcctctccc ttttcacttc aaggcbgtct tgcattgtta tgtaactatt tcatgggcct |
| 205501 | agatctcatt tttcttaata aagtttaagc tgtagaaagg aattcatata tttccttgat |
| 205561 | ttcttttaaa cacttaacac attaagaaca taaaacatac ccagtaatta cttaaagaaa |
| 205621 | aaaaagacta agtaaaaggg acaatgaaag aataatagaa ataaaatgaa tttgtttatc |
| 205681 | agccagaatt ttgggtgggt cctcctcata gctatgtata gcaatctata atttcagtgc |
| 205741 | tatttctacc tacttctaat aaaaatattc ccacagtaaa ggggcaaggg gaaattccac |
| 205801 | cattttgcaa cattttggtg attagaattg ttgagatatg gttttagaca gagtttcctt |
| 205861 | cttattcttg ggctggtgat tttaagattc atttgtgtgt tttaggtcac attctcccca |
| 205921 | aatctcattt ttgctcaaca caagaacagt aagagtaagc tgggatgcct tagtgatatg |
| 205981 | gcctggaact ctgcagccta cataatacta tccctacctt caggtattag tgttgctaat |
| 206041 | aacatgctca actgattcat taaaacaaag gacaaaaaaa taaacaaaca taaaatcatt |
| 206101 | ttcatagagc ttaaagagac tatccagaga gacatttata aagttaccat tataaaatgc |
| 206161 | ttcacactaa aaaagaattc tttacaataa tttggttgta aattgtgtaa aattcttttt |
| 206221 | ccctccatca attttagatt tattggttgg agccttgtaa gtcagactga caaaagacag |
| 206281 | attaacaaga gaaaaacaag cagaagttta ttaccatgta tatccctctt attcatggga |
| 206341 | gatgagtaac tgaaagagga ggttagaact tgagctcaca tagcatctta acaaaagaac |
| 206401 | agtacatttt taaagaagtg acaagacaaa ggaaaaggac tttgagtttc taggatggca |
| 206461 | aatgatggga aagcaaatat atgggggaac taatggaaaa taaggactat ttagtaaagc |
| 206521 | ttgtcatgca gattcctctg gtgccctctc ttgactgata agtctggaat agtcttcagt |
| 206581 | gattaacttc tgtccttcca ggtaagaggg gaatgaaaat aataccttta tatatttatc |
| 206641 | tcctgctttt aggcaaaggg ggagagcaga gagcttttct tgtatctgct gcttcttgat |
| 206701 | tgctttcatc ttgaaataat ctttatacca tatttttggc acattttggc ataatatggc |
| 206761 | acataatcct gacatggcat attttggggt gccatattct gctaccctcc aaattgtctc |
| 206821 | tacccttcta ttatcttagc agataatagt acaacctccc aaattttggt ttccttctgt |
| 206881 | gaggaaaaca cagtttgtgc tgtgaccagt attgtagctc ctggggaaac ctgtgtggtc |
| 206941 | tcctctctct tctccctgct actgtaatac taacaaaaag aatgtttaaa ccataaattg |
| 207001 | aatcttaaag caatcaagta caaatcagag ttaggaaaag ttttaatcag caggtagaaa |
| 207061 | aaggatctag tgtgctttta tttttaaatt tttcattaat ccactaaagc ccagcttgtt |
| 207121 | atttttacct tgtttcatct aaaattaccc taagatgctg gcatgacctc tagggtgtga |
| 207181 | ctaaaatgtg tttgagtctt tatttgacat ttaagccagc catgaaaatt agcattggtt |
| 207241 | tagagctcca gggagcaaag aagtcatcct gagtctgaag aatttctgtg gctgtaactt |
| 207301 | ggaatttcat acaaatttga cctgtccaag acttgcttta attattcact aacaccagca |
| 207361 | tctggcttta acacatcttc atgagtatca tcagaactag agaaaaaaaa ttcccttaag |
| 207421 | aaatttttgt acataggtca ctctcatgtt tgttgctaat aaaaatttta gaggtcttag |
| 207481 | taatctaaaa aatatatatt ttacctgtaa agtggtacta tccaaaagga actataccaa |
| 207541 | aatacgtcag acataaaagt taagaggtta tgaaaatgta catgcaattc tctttttgct |
| 207601 | tgttcttagt ttatttactg cttgttttca gatagtcagg tagttactct ttagagatgt |
| 207661 | aaaataaagt atggacatga aaagtagtta tttaccaaag gcaattatca agtatcaaaa |
| 207721 | catgcagagt acttactgcc agttcaggga gacaacaaaa caattatata taatatagta |
| 207781 | gctttttagc ccaatcttcc aataattgct aggccaacat gctaaattta agagttcttt |
| 207841 | acctcttaaa tttagtagtc tcagtaaata aaatttgttt agttctgcag gggcagaaag |
| 207901 | atgtgatact ttgtccctat cataaggtca aggacaacac ttctctaatg aaagagggtt |
| 207961 | cacaagagaa aaacataaca catttcttta accaaagttt tatatgacat ggaagccttc |
| 208021 | agaatgaaga tttgaagacc caaggaaaaa ctgtccattt ttatgcttag attcaataaa |
| 208081 | gtgtgaacag ccatgtagaa gtgttattag acaaacaggg aattatctaa tagtaatgaa |
| 208141 | ctgagtgggg aaacccagca atacctgtcc agattcttct tggcctcact gttgtaacat |
| 208201 | ctcttcaccc tcctggctta tttggtatag ttccttttag acagtactac ttcatagata |
| 208261 | aaatatatat attttgatta caaagactac aatttttgtc agcaacaaac atgagagtgt |
| 208321 | ttcttcctcc cttgacctac tgttgaccaa gactaggtca tagaatttct ttatggccac |
| 208381 | cttctagaca gaaaggtaga gaaggttaga gtaataattc ttgtttttat ggccggcttt |
| 208441 | ggggaaaagg gggttttagt ttctacggcc tgccctgggg aaggggaatt ctgaattcta |
| 208501 | tggcbggttt tggggaagaa caaggggtga gaggtaggag agcaggagaa ggtcagagag |
| 208561 | accttggctc tgatgctgct actgaggcct tccagtgtcc tttagttcaa ggtgctcggc |
| 208621 | atgccaaagc agcacattca ggatatcatt ttctgagccc aacagttcca tattctgccc |
| 208681 | ccaacttgtt gcacattgga attgttcatg gacttacaaa aatattgatg ccatatccca |
| 208741 | gccctagaga ttgtgactta atttagctgt ccccaacctt tctgatacca gggaccagtg |
| 208801 | tcatggatga caatttttcc atatactggg gcbgggtgag gagatggttt caggatgatt |
| 208861 | caagtgcatt acatttattc tgcacattgt aatatataat gaaataatta tacaactcac |
| 208921 | cataatgtgg aatcagtggg agccctgagc ttgttttcct gcaactatac gatcccatct |
| 208981 | gggggtgatg ggacacagtg acagatcatc aggaattaga ttctcataag gagttcacaa |
| 209041 | cctagatctc tcgcatgtgc agttcacaat agggtttgca cccctatgaa aatctaatgt |
| 209101 | gactgctgat ctgacagcag gaggagctca ggtggtaata tgagttagca atgggtagtg |
| 209161 | gctgtaaata cagatgaagc ttcactcact cgcccaccac ttgcctgctg tgtggcctgg |
| 209221 | ttcctgacaa tcatctggtc ttctcactgg tattcatttg ttcaattagc atttgttgaa |
| 209281 | attcacatct atgccaggtg ctaaacccat atctgtgcca gtggccacga cctgacagtc |
| 209341 | atctggtctt ctcactgatg tccattcatt caattagcat atgttgaaat tcacatctat |
| 209401 | gccaggtgct atattaagta ctgggaaatt agtacaaaaa atatagacct tggagattcc |
| 209461 | agtgcaatgg taaaaatgga ctagaaagca acacactggg ataactactg cagaaaggtc |
| 209521 | agaggttaag tcacactacc tagggggtgg aggagattgt tggaaagaaa gaaaggatca |
| 209581 | ggaaaggttt tgtggagaag ctgtagtgtt ggcctaactc tagaactgaa gataaagaaa |
| 209641 | aggaagaaaa taggtgtagg agaagagaaa gggtatctta gagaaatgaa acagcatatg |
| 209701 | taaaagcctg gatgcattag aaatcaactt gcagccagcc tctcatctta tgttttctct |
| 209761 | aacttatctt gtatatcatt gttgaaacat ttttctcaca gcactgtatt acacccatca |
| 209821 | atttcctgct caaaaatact ctggatcctt actaataaca gaatgaattc tgcactgcag |
| 209881 | acgtgacatt agggcatgcc agatcctcct ctaaagcatt taaactgtcc tcttcattgt |
| 209941 | ctatctgaag accccaggta tgttttcatc tcttcatttt tgcttatatc attcccctgc |
| 210001 | accagaatgt ccctcctact cctttgacca tgtaactgaa tcctcccttt gttcagggtt |
| 210061 | ccttgatacc actccatctc tcatctcaat cttttagcat ttatgtttac ttttactatt |
| 210121 | cttgtttgtt ggtaattaga tggtgcccta aatatttatc tatatcttta ctttttaatg |
| 210181 | aaattgtaaa ttccttacag aaaagcatag gaccacattc ctttatctct ctttctgcat |
| 210241 | atggtacaat gccttaaaat aaacaaaatt tatttttatt actgataaca aaactgaatg |
| 210301 | acaataatat aatgagtttg gaaaatggag aaaccaagca tttgcataat tgcctagtct |
| 210361 | ttgatcattg atgcacacta cttttacata attataatga gagtatacaa attgttctgt |
| 210421 | attctgaaat tttacttgac attgtatcat caacatttgc tatgaaaaat aataaatatc |
| 210481 | agcbgggcaa ggtggctgac acctataatc ccagcacttt gagaggccaa ggtgggcgga |
| 210541 | tcacttgagg tcaggagttc gaaaacagcc tggccaaaag ggtgaaaccc tgtttctact |
| 210601 | aaaaatacac aaaaactagc tgggtgtagt ggcacaagcc tgtagtctta gctacttggg |
| 210661 | aggctgagac atgagaatct cttgaaccca ggaggcagag gttgatttga gccaagattg |
| 210721 | caccactgca ctccagcctg ggcagacaga gtgagtctct gtctcaaaaa aaaaaaaaaa |
| 210781 | aagataaata tctttggata ataactaaaa gcatcattta cctggtgtgc caggtactgc |
| 210841 | tttgcttaca ctgtttcaac ttttgtatgt atgtccttat tccaattcca tgaggtaaca |
| 210901 | ctggtgttat cctggggaaa ctgggagcac tgagctcaaa aagagtagag ctgtgctact |
| 210961 | tactaggtga ccttggatac ttattttact tagcgaatcc tcagtttcct tatctctgag |
| 211021 | atggggataa taatggttcc cacctttcaa gattttgagg ggatgaagtg agatgatgga |
| 211081 | cacgaatagt gcctggcaga tggccaacac tcagtgtgct ttagctcttg ctgccgtcac |
| 211141 | tgttgcbggt cccattttac aagtcaggaa actggatcaa aggcatttaa taactctccc |
| 211201 | caaatcacac acctggtgtg tgacagagcc aggattcaaa cccctgtccc aattatgaca |
| 211261 | tgagataggg atcacagaat ttaatacgat gttgtccaag tgtaacattt tacaatttag |
| 211321 | tgctacagag aacatgtgca gagttttgag agaaaacaca gttggatatc tacagattat |
| 211381 | cttctgcttg gagagaaaga gaaattgaga gagatagaga gagagagatt cccaggggcc |
| 211441 | cgagctctct acagcagcac agcatggctg ggtcagggct caggatagaa ttcctgactg |
| 211501 | cctcctccca ggcggtgtca taactggggc ttctgaagcc caacacctgg gccctctcct |
| 211561 | tgggtctcaa atggagggtt gaatcctctc atgctcactt actaacaaga acaattccca |
| 211621 | ctttcaatgg ggtaacagtc tgaggggaaa tgggctaggg ttttgcctgg aagtacctag |
| 211681 | ttatttctta actttgtagt ttggagattc agcataaaaa ctgaagtcag ccctgttagt |
| 211741 | agtattgctg cttatacagc cgtccaccct tatcagaggg gaataccttc caaggctccc |
| 211801 | cgtggattcc taaaaccaag gatactactg aacctgattg ctgtcaatca gaacatattc |
| 211861 | ctgtttatgt cttccaccac acatttaatg ccttttctat cttaactaac cacttatcac |
| 211921 | acactgtggc cataactttt gcagtttgaa gtacaacaga aaaactagca agaatttctt |
| 211981 | ttcccttctt cacaatttca ggaagagaag acgtcttacc atagatctta gcaacctcag |
| 212041 | catacttttt tttttctgcc ctcattaggt tgagaacttt cccttattca cttaaaggag |
| 212101 | gcactttgtg gcttctcttt ggcatatcca aattgccagc atcactaaac ttatgctttg |
| 212161 | ggggcattat taagtagaat aagggttact ggaacataag cattgtgata ctgtgacagt |
| 212221 | caatctgaga acccagaggg ctactaacag gaggagagtg cagactgtgt ggatttgctg |
| 212281 | aacaaaggga agattcactt cccaggctgg acagcacagg atggcatgag atttcatcat |
| 212341 | gctactcagg acagcacaca atttaaaact tatgaattgt ttatttctgg aattttccat |
| 212401 | taaatatttt tggactgcag ttgactactt gtaacaaact gtggaaggca aaactgcaga |
| 212461 | taagggggaa ctactgtaat agttactgct tatactgctc ttgctatttg ccatggacta |
| 212521 | agctaaacac attacataca ttattttcta taatggatgt gacaatccta cagagtagct |
| 212581 | actattatct ccattttaca gatgaagcaa ctgggcatct taaccattcc agctactgcc |
| 212641 | atcagcattt gccttaggaa aaaaatgtgt tttgaataat cacttccagt atttgggatg |
| 212701 | attttcaata attctaccag cctaactatg aagttatatt atctaggagg cctctagttc |
| 212761 | ttatttttaa tctttgtacc ttcgagtagc ttcttacctc tgtaactcac tcccacagta |
| 212821 | atatttcatg agaagccaga ttctctggac tattggttaa attcttttag atggcagtgt |
| 212881 | ttgttatgaa aagacttttc attcttctgg tcccttcact ggaaacagct agaaagtaca |
| 212941 | agttttgatt acagttgcct aaacaccagt tttgaaatgt ctttcatttg cttgagcaat |
| 213001 | aaatttgttg ctcaatcata ctatcaaatt gaaaacaaga ggaaaggaaa gtcaacctaa |
| 213061 | cctaacccaa aaggggaaca tgtttcctgg gatgagaatg tgtcaaaggc agcatagcat |
| 213121 | gacctaacac ttctgtgtaa tcgtggttgt atcatgtgtt atgggtaaat tatctattct |
| 213181 | gtaagttagt ccagtgtgtg cttcttcaac taactgtgaa agcattcttg aaagaccatg |
| 213241 | aaaccttatt attctaatta ccaacacaac ggattgataa atgaatgatg aggccaggta |
| 213301 | ttattgtcat taaatagacc tcgctcaatt atttatcaat gagtctgggg ttacagctca |
| 213361 | gagagctgtg catattgtag cacggtttgc ttcagctctg agacctgaag accagattac |
| 213421 | agtcttggca ggtggctttt gttgaatgca attcaagggg gtggtacaca tttgtttcag |
| 213481 | tcagacattt ggatggtttg aggccaacag atattcaaag tttatccaca aaagcctatt |
| 213541 | tcattttaac catgtagaaa aatctcttta atttattcag ataaatacaa atgagtagga |
| 213601 | gaatggatta gctgtagaaa atcatacatg taaagatgtt ttcttgcatc ttagtgttct |
| 213661 | aaagactgtt ctttattttc ttagattctg ctatctgtca tggacacttc actgttctgt |
| 213721 | attgatcaat gtttatcttc atatgagatt tgtttttgta tcttctttct aattgtacaa |
| 213781 | tgaaatatgt tttctggtta agaaatacgt tatcaaggag gatagaattg ggtgatacta |
| 213841 | gtttttttcc atcctgaaat catgccagct cattgcaaag gtcagccctc aaaatagtat |
| 213901 | taaggcatgc cctctccttg agtctctgat accttgcagc ccaatgtccc ttcttgaagg |
| 213961 | cctgtcacct gcaagagtac tttcttacat cctatgagca tcagatatgg gggaatgaag |
| 214021 | aatctcttct tgctaaacaa atattcttgg tccaccttct gccccctggc tttattttaa |
| 214081 | acccatcccc aacatatctg atgagttgag tctagtaatt ctgtcttcag tgggctatgg |
| 214141 | ggagcaggga ttagctaaaa tggcbgttca gggacctctt tggtataaaa cttggtgggg |
| 214201 | gtgctggcag ggatctctca gagaatgaag gaggggatag agcattagag aagaaaagaa |
| 214261 | attcatacct gagattatgt aatagataat agagtctgga ctttgtttaa aaaccagaat |
| 214321 | ctaagatata tatgttgagg ctagggcggg gtagtaattg aaagagaaat atgaaactat |
| 214381 | acctagcgtc aaggaaaact aacagtaatt gaagagacaa aaataaaact tgtttatatt |
| 214441 | gttcaaagga aaatgatatt tataacatct gacaaaaact ttattaatgc catgtagaca |
| 214501 | gggttttata ttattaactg agtttataat taattttgta ctgattttaa tttgactgtt |
| 214561 | accttggctt ccttctctgt ttcttaagtt ttgctagttt aatgttcacg agctcacaaa |
| 214621 | attaaaaatc ttggtagaaa tatggagtag ctatttcttt acatcctttt tagaacaaag |
| 214681 | ttgtgctttc gaattgacca cttatctaga aagtgatata aatctaataa gcaaaactgg |
| 214741 | atgtctttta aattatctgt taaataacta gagcagtgga gatgtcctga aagtgagcac |
| 214801 | tttattacat atgaaaaaca aattttaatt ttactactaa gaagaatttc catttctcta |
| 214861 | tttatatgtg ttcattattc tagagattaa tatgataact cctatgtata cttagttttg |
| 214921 | tgaatttcaa aattcttgat gccaaataac tgaagtatag taaattctgc cgtaacatga |
| 214981 | tgtatgggtt cctaaaaatc ccgacactat gcaaaattgg gcaataaaaa cacattttac |
| 215041 | taaaaaaatg gggataggca cacaatacct agaaatatca ctagtggcac attacaaaaa |
| 215101 | atatgggaat ttagtaacaa tgacagcaat attttacaca tgttaatggt taagaaattt |
| 215161 | acaataaata tggcagttta ctttgaaaaa gacctaacgt tccctgtgga agttggcctt |
| 215221 | caaaagggtg cagcttgtga gtttttgaga agtgttggga ggagagtcgt tgttgtccca |
| 215281 | gttgtgagca ggtggggctt ttctacctgt gtggggaagt gtggtagctg atggatatgg |
| 215341 | tatgtggatt ttgtgtattc ctacgctgct tggtttagct gggtgcagtt ttctgcattt |
| 215401 | acctggcgtt tcttgtggat agaatcacat ataagcaaat cccagacttg tgttgtgatc |
| 215461 | aaattgttcc gtaatatatc aatcatgtta gaacaaggtc acggtttcaa aaccggcatt |
| 215521 | gccgtagaat tgactgtgta ataatccctt caacacgttc acatctatga tcccaccttg |
| 215581 | ttcttgccac catcttagca agcaggggtg cgattgcctg accatttgtt tgttttctgg |
| 215641 | gaaatcttga cctattaaaa gccacacagt tactgaggaa acctgtgtca gaccttgcct |
| 215701 | aggtctgtgt gagtctgagt caggtttgct ttcctctgaa aaatcagaca ctcatgtatg |
| 215761 | tgggacaaag agcatcagaa acatgggcaa ggtcatagag acagatccca gagactggat |
| 215821 | tagaacccaa cttgcttgat tttctgctta gaggcttcag ttcaccattt tagaaagtgc |
| 215881 | ctgctggctg ccaccctccc tcctgtctta tcacctctaa aagacattgt taatgcttag |
| 215941 | acaggatgtg ttttcttcat tgcatggatc taaaaaccca gccatgagaa cttagttttt |
| 216001 | aatttttgcc attgctttat tggatatttt agaatgtgca aaaggtttta gaaattgttt |
| 216061 | ttcaactaat tttaccagca cagaggtcat gtcaaatgtc agggtgtttg acaacaaggc |
| 216121 | caatctagtc tatcaattga gtttcagaga agattaatga taaagggtgc aatcccaaag |
| 216181 | tctgggaatg actttgcttt caagcattaa ttattgtgga aaataaaatt attagatact |
| 216241 | cccttgtcta catctacccc aaactatgtc aatcagctct atttctagaa ggtttacatt |
| 216301 | tattaaacac tccctgtggt caatgcaatg gtcaatgcta tggaagttac aacaagacaa |
| 216361 | gatattgtgt ccgctatcag gaaactgatt tgggagttct gtttgatgac cgaagagagt |
| 216421 | ctttaccact gtgcctatat ttttatgcca gaatatgggt gggtgcacat gtacaggtga |
| 216481 | gtaatgaggc cccactccct agtattagtt aagttagcag gtgtgtgctg tttttttggt |
| 216541 | cactgtgtat tgcaaaattc tgagtttggc actgatacta aggaacatct tacagaaaaa |
| 216601 | attccccact gacccttctt ttttttttct ttttgaaaca gaatctcact ctgttgcccc |
| 216661 | aggctagaat gcagtggcac aatcacagct cactgcaacc tccgcctccc aggttcaagt |
| 216721 | gatcctcctg cctcagcttc cccagtagct gggactacag gtacacacca ccacgcctgg |
| 216781 | tcgatttttg tatttttagt agaggcaggg tttcaccatg ttggccaggg tggtctcgaa |
| 216841 | ctcctgacct caagtgatcc acccaccttg gcttcccaaa gtgctgggat tacaggtatg |
| 216901 | agccactgtg cctagccaga cccttcattt ttaaaagttg actttatatg ttaataggag |
| 216961 | tttaattata gtcatatgtt gaagggtctt ttctgtaaaa ccacatattc caggagccat |
| 217021 | ccaactgagg acagcattgc acttgtggaa ccacaatcca ggcttgggtt atctgtttct |
| 217081 | atcatgacag aggctactcc atcacttaaa ctatatgttc tgcagccccc gagagctgaa |
| 217141 | gacaagcctg gattgcctct atttgtaatt ttcttttttg taaatttttt tttggtaaat |
| 217201 | tgtttttcaa acaaacaaca aaaatgactg tcccagtcag ttcaagctga tagaacaaat |
| 217261 | ggccatagac cgggtggctt aaacaacagc catttatttc tcacagtgct agaggctgaa |
| 217321 | agtctaaaat cgaagtgcca gcatggtcgg gctctggcaa gggccttctc ccaggttgca |
| 217381 | gatggctggc tccttgtgtc ttcatgtagt ggaagcaggg ctagctagct ctctggccac |
| 217441 | tacttacaag ggcactaatt ccattcatga gggttctacc cccatggcca attacctcct |
| 217501 | aaatacccca cctccaaata ccatcatgtt ggagattaga tttcaacata cagattttga |
| 217561 | tagggacaaa aacattcagt ctataacaat gatcaaataa ggttacaaaa attatagtgt |
| 217621 | atgtagcatg cttgcattat gttttgatga attaatgttt tttattatgc ataccaaaga |
| 217681 | aactaatgca actgctgtaa atgtataaat atttgggagt aatgttaaag tttacattta |
| 217741 | aagccctttc taaatgtgag gccgaaagta ttccttttct ctctctttct cttctcaacc |
| 217801 | ccatttactt tacagaatga tttttctgtt ttaagtcata attgcttttt ttttacagga |
| 217861 | ttaagtacca ggtcattaag tgtgttctat gtcagtttga cctaacctct ctgccctttt |
| 217921 | cttttttcta aaggaattct ttaaaaagaa gaagaggaag tattttaaaa taaacatttg |
| 217981 | actgccagaa tgtgatgtaa acttcaggtt caattctaca cttgatagaa atgaaaagaa |
| 218041 | ctagtgttct ctattttgac atttagcatc aattcccttt cagtgattcc agctgattag |
| 218101 | taagtgagac taaaatatct tactcttcct ggtcagcatt taaaaattta aacatggatt |
| 218161 | gaaaatttct aggttatgaa atacagtcta ggttcacctg tgaaggatct agaagaaata |
| 218221 | agactgcaga cctggtaaat tcaaatgtcc actacttctg tattctagtc attgcaactc |
| 218281 | acctcctttt atcccttatg ttatttttat tttctcaggg aggcaaaaaa atttgtgagt |
| 218341 | gaaaatgaag gggctcttgg gaaaggaaaa ggaaaacggt ggtttgcatt taagatgatg |
| 218401 | atggccaagg taggtatttt tatagttgtc tggtttatgg catattgcct ctaagaagaa |
| 218461 | cactggttct tttctaaagg ggtgaaaaaa atcttacatt ttgtatatat aaaacacaaa |
| 218521 | tactcataca gtatatacac agtatatctg tggtattgaa atttacaggg gagatttaaa |
| 218581 | aaattgttct tagaagagtt aaaatgaaaa ataagtcaag aaatacagat ttatggcaaa |
| 218641 | agacttactt gtgtgacttt agcacttcca aatataaaag attgcatcct ttttcatttt |
| 218701 | cattctctgg aagtgaaggg gttctggtca agttgttttt ctcaaagtat ctgtatagtt |
| 218761 | tttttctacc aggaacatca actatacatt ggacagaaaa taagaaaaca ataaatgata |
| 218821 | tactgataca aatatagatt ttggggaaag acagaaaagc aactaacatt tatttaacac |
| 218881 | ttactatgtg ccaagcactg aattgaattc ttttataaag ttatatttaa ttcttacagt |
| 218941 | aaagtaggaa catagatatt attatctcct ctttacaaat gttataccaa ctgtaagcta |
| 219001 | aatgtattcc cataaagttt tgtgaataaa gttcatatat cctaactttc atatatatat |
| 219061 | atactcacat acaatagatt aattttgtct gtttctacag tttaaataaa ttgaatcata |
| 219121 | tagtatctat gcatttgttt ctggcttctt tgatgcaatg ttatgttcac aagatatatc |
| 219181 | cacgttgttg tgggagttat acttcatttt tattcatgtg ttgtagtcta ttgtataaag |
| 219241 | atgctgtgac aatgtattta cccattctat tgtagatgga catttgggct gtttctagtt |
| 219301 | atgagtaatc tttttttttt ttgttcattt ttaaaattaa ctcatacaca attacttgtc |
| 219361 | ttctggtttg ttgaaacaat aatgcagaca tttgctacaa caatgactgt taaaatggct |
| 219421 | ggccacaaaa tctccagcaa tacattaatc tgaagggcat tcttgatgaa gttgactaat |
| 219481 | tttgccattt tcatccacct tatagtattt caggacagcc agcttaacct tccttttctt |
| 219541 | atgcttattc ttctttggag tggtgtaaga cttcttcctt ttcttagcac caccatgaag |
| 219601 | tctcaacaca agatgaagag tagactcctt ttgaatgttg tacccagaca aagcacatcc |
| 219661 | atcttccagt ttcttgccaa caaagatcag tctttgctga tcagtagaaa ttcctgtctt |
| 219721 | atcctagatc ttggacttcg cattttctat tgtatccaag ggttcaaact ccagagtgat |
| 219781 | ggtctttccc ataagggttt ttaaagaaaa tctgtgtttt ggtggtggtt ccactgcaga |
| 219841 | aggcagatgg gaaaaaaagc gtgggtgatc ttttgaactt cagacttgtc tcatgctatt |
| 219901 | atttagacaa tagtgcctgt ccagctccca cttcatccca gcatttgcat acatgcaaaa |
| 219961 | ttctctgcca cattctcatt cttcctggtg gtcctcttga ctatggatga cccaacattg |
| 220021 | gagttaaata gaaagaaggc cttttgcaaa atgattttat gacttgttgt cttctctttc |
| 220081 | aattaagctg accaatctgc ctctaccaga atgcagtgtc tcatgtcata caacttacca |
| 220141 | tcttgtattg aactcaccta tttgtacaca gactatcctg gtagatcaga aggctttcaa |
| 220201 | gacaggacct ggaaataaaa gacagaaaat aaaatttatt ttgtttatta tttttattat |
| 220261 | ttattttatt taattattca tattgagtcc ttcatacaat gaaaactaat taaacacata |
| 220321 | ctctgttcca atcaccctat tatgtgctaa tcccctgctt tcttattgtg ttatataata |
| 220381 | aatagcbgtt tctgagaggc cagcatattt tccataaaac aggggatcga taaacatttt |
| 220441 | tttgttgaat taaattggtc tggggttagt ggagaacagg gaaacaagtg aaacctactg |
| 220501 | tttccccctt tgactagaaa gtagtatttg ctgccagaga gacatttcca agcttactgc |
| 220561 | attgtaatgt tttgaggtgg gggataaaca tcttgacaaa tcaagaggtt ggactttact |
| 220621 | tttgtatttg actttctttt cagaaatggg caaaattcct ccgtgatttt gagaacttca |
| 220681 | aagctgcgtg tgtcccatgg gaaaataaaa tcaaggctat tgaaagtaag tccttatcag |
| 220741 | atctcaggtg gagaaggttg tcttagagaa gtatctcata agcttatcag atgcctttgt |
| 220801 | cagtttctgg acttttggga agactagaaa caaacaaaca aacaaacaaa aaccaaatca |
| 220861 | acaaaaactt tgtttatgcc tgctttcgtc actgtgaaga tttgctagca cttaccaagg |
| 220921 | acaatactgg ggaccccact ctcgtgttct accttagtcc aaaacattac cattctaacc |
| 220981 | tggttttctc tcctatacag ggaagtcact attgtgcaca atgacattga atcatcttac |
| 221041 | ttggaaggca gagattttat tttggcaatg aacatgacat tgagaatata tactctcatg |
| 221101 | aatgttagac agtaatcaaa atgaacaatg ttttaacaag tgaagaagtc ttagtctggg |
| 221161 | cgcagtggtt cacgcctgta atcccagcac tttgggaggt tgaggtgtgc agatcacttg |
| 221221 | aggtcaggag ttcgagacca gcctggccag cgtggtgaat ccccgtctct actaaaagta |
| 221281 | caaaaggccg ggcgcggtgg ctcacgcctg taatcccagc actttgggag gccgaggcgg |
| 221341 | gcggatcacg aggtcaggag atcgagacca tcccggctaa aacggtgaaa ccccgtctct |
| 221401 | actaaaaata caaaaaatta gccgggcgta gtggcgggcg cctgtagtcc cagctacttg |
| 221461 | ggaggctgag gcaggagaat ggcgtgaacc caggaggcgg agcttgcagt gagccgagat |
| 221521 | cccgccactg cactccagcc tgggcgacag agcgagactc cgtctcacaa aaaaaaaaaa |
| 221581 | ataaaaaaaa taaaaaataa aaataaaagt acaaaaaatt agccaggcct ggtgcaaaag |
| 221641 | cctgtagtcc cagctactca ggaagctgaa gcaagagaat cacttgaacc cgagaggcgg |
| 221701 | aggttgcagt gagccgagat cacactactg cactccaacc tgggtgacac agccagactc |
| 221761 | tgtttcaaaa aaaaaaaaaa agaaagaaag aaagaaaaaa aacgaagtct taaaacctca |
| 221821 | ttttcaaatc tctgaagtag aataagcaat agtcatccag gagcttgcta tctgaatatt |
| 221881 | cacattgtgc ctttattttg tagaatattc ataggaagga aggaaggaag gaaggaagga |
| 221941 | gaatatagcc aatcaaaaac ctatttagtt tagtgcttta ggcttagaat aaggcaagta |
| 222001 | aacattttaa aatattgtaa ataaaaatag taaattgttt tttaaaataa taatctaaaa |
| 222061 | aaagggataa catttgaaac aattttgagc acaatctttc catattctgg tttctaacgg |
| 222121 | tttcaactag gatctgattt ccatccattc ctctctagat tggaggcagg ttttcctgct |
| 222181 | ggttaagtgt ggcgttgtat aattaaaatg gaggctccat agggagataa cacagctgat |
| 222241 | ggatagcttg ctgacatgat tgggcatgtt ttaggagtgg ctaaccctcg gttccctgtc |
| 222301 | tctcaatatc agatgggaat tattaagtgg caatgaatta aaatacatca gctcattaat |
| 222361 | tgtatcccct tccacacatt cccagtgcca tttctctctt gctttaattt cctccttaac |
| 222421 | acttaccgct atctaaaatg ctatatcttt aatttaattt tttgatgtaa tcttaattca |
| 222481 | ttttatgttc atttctccca atagatggga aatttagttt tgttcactcc tgcattccac |
| 222541 | ttggcactta ataaatatgt attgaataaa attacagaca aactcgctaa atgtctacca |
| 222601 | atctaggaaa aaaagaaagg aatttagata catcaaaaaa gagaagacat catggatttc |
| 222661 | ttaggtttca actgtacagc tcaaatgtgg taggttgaga ggcactgaaa aacaaaaggc |
| 222721 | catttggagc attacggtcc tgatacaata tatatagtaa gaactctgaa tttggtcttt |
| 222781 | gctttgctgc agattagctg tttgacttct agccagtcat ttaactcctc tggacctcag |
| 222841 | tctactaatt tataaaattg tagggctgag tcttgatcag ttgttagata aatcccaaga |
| 222901 | aatttgtaat taatgatagg taaatatata catttgaaaa ttatttttaa agcacactag |
| 222961 | agcaggttcc agcctatcct aatagtaata tcatgctcat ttgtgttcca ataggctata |
| 223021 | gccagtgaaa caaaaacaga cagaattatt tttattgcca gaaattgcat gcagcaagag |
| 223081 | ttacacatat gaccaccatc catcatgagt gtcacagtag aaaagacaga ttgagagcta |
| 223141 | tccaataata accttcagtt atctcctctc ttcttctctt cttctagtaa atttggaaag |
| 223201 | ccacgctgga aaattgaggc aaatgtagat tatttttctt tccatatata atttatgatt |
| 223261 | tgattagttt gtcatgactt cttcactgtt aatggtgctt tgtctcaact gtgaagaact |
| 223321 | gagctttatt ttttatttta tcaatattca ccacaccacc atttattgag taccaccatg |
| 223381 | agtcagatgc tctacaaaca ttgtttcatt taatcttcag tgttcctgca atgtgcacat |
| 223441 | taaccttcct attttatagt tgtgattaaa taactgtccc agaattagta gcagagctga |
| 223501 | aatataaggc aaaatctgtc tgggttcaaa acgtatgctt ttccatgtgt taggctgctt |
| 223561 | tctcctttag ggaaggtttt acatactttt tctttttaaa tatgttttgc accagttcat |
| 223621 | aaaaagtatt ctacgaggcc gatggcagga tgagtagtta tagattttct tgatcgttat |
| 223681 | caaggaatta tttttcattt tctcatgtat ttacttttta gtgagtgatt tatttaatta |
| 223741 | tatgttttgt attctatagg atgcttaatg tactgatttt aatgataacc atgaggcttt |
| 223801 | gggctatagt attagttgaa aaaatatgaa acctatgtaa gaatatatta ttagagcact |
| 223861 | ggattggact atcttataaa aagtacccta ctaaatatag gtaaagcatg tatctgtgat |
| 223921 | cacctttgaa actgaactta atgcaaaatg ggaagaaata ggacatgcaa tggattttct |
| 223981 | gagttgagga ctttttgacc cctaccacct ctgtagtttg gacaatctgt tgaaatctct |
| 224041 | ggttctgttt ccacatttgg ggctactaca atatctacaa catcttgggt ttaactagtc |
| 224101 | actatacaag atcatttcca gctcaacatt ctatgatgtg taagttacag tcagttttac |
| 224161 | atgtatatgt aggcatgtga aattcattaa tcatactttt gattgataaa tcctcaaaaa |
| 224221 | tttgaatgca aaacagattt atactaagag gggactatat aattgccttc tcaaattctt |
| 224281 | tttgaaataa agttggttaa gtgtgtaccc tacttataga atacatattt aattataaaa |
| 224341 | cggatataaa tgcttttctc ttaaataaat ggccatttaa tcttacaaaa ggttcagaag |
| 224401 | tatcattgta ttccatttcg acatcagaat catctaatat atatcttatt taatcctcag |
| 224461 | aataagcctg acattattga ttaactgagc aaatagtgga tgcctaacat gtttctaggc |
| 224521 | actattgcta ctgccaaaga cagcagagac tctaccctca ttgatcttac atccaaatgt |
| 224581 | gttaatgtat gtatgcttgg atggggaaga cagaatataa tataagcaaa taaataatga |
| 224641 | cggtaattgt ggattgtagt aagtggcatg gtcaaaagag gagcbgttgc cttccattcc |
| 224701 | caacagcctt tctaatccac ttttccccac tctctgctca gtatctagat aataccaaat |
| 224761 | aagtattttt aaaatgtggc taaaggagtc caaaattaac agcctttact gagttttccc |
| 224821 | aaagctctgt ccaataattt gcagaatcaa gagcgggacc caaattagag tctaaaattc |
| 224881 | atactgtttc catcttgatt ccagtaagac ctagatctct ttattagtgt gaagcggatt |
| 224941 | ctcacctgat gactcccaac tctgtttcac ttaaaaaata gatttgataa ttcctgccac |
| 225001 | agaggatact gtgacgttat gcatcacttg ggaatactgt ggggttttac tgtcacgtca |
| 225061 | gccttaaggg gatcatgcct aaaactcgat ggttgctcct gtggacacat cttattcatc |
| 225121 | tcattgaact gtgtattttt ctgaagcaac tgcatgtttg tacccccagg aaatataatc |
| 225181 | tatacccatt gtgatgccaa cattattatt aagaccataa attaaataat tgaatgtatt |
| 225241 | tctagcbggg gattacaaat ttttctcagg atcaaaatct actttttcct aatagtagat |
| 225301 | tgcatcatca aaattatagt ttcagaaacc tatttttttt cagacaggaa atagctttgg |
| 225361 | ataggcaagc caaagcacag ggcttaaacc ttatgtttcc ttgctgccct aaggattcta |
| 225421 | gaaaatacaa atcaaatttg tcacatttaa ttccctaccc ctaaatagtt cgaaaaaatt |
| 225481 | gtgcttcaca ttctagccta acatgatggt atttatattg tagtcacaca gcaggtcatt |
| 225541 | taattggtct tttcccattt agaaatactc atatttatct gcttgtctta ttacccatct |
| 225601 | attcaagagg aaatgatgtg ttatgacaga ctacaaatta ctttgcctta ataataaaca |
| 225661 | accagaaaga gagttgaaat tttaacttta atttgaaaga aacaagaata ttcaggaaca |
| 225721 | agaatatgct taagcataaa gcctcagttt aatacgtatc agagactctg ttttttggtt |
| 225781 | gttgttgttg tttgtttgtt tgtttgtttt ttgagacaga gtcttgctct gttgcccagg |
| 225841 | ctggagtgca gtggcgcaat ctcggctcac tgcaaacctc tgcctcctgg gttcaagtga |
| 225901 | ttctcctgac tcagcctcct gagtaggtgg gattacaggc tcctgccact atgcccagct |
| 225961 | aatttttgta tttttagtag agacggggtt tcaacatgtt gaccaggcag gtcttgaact |
| 226021 | cctgacctcg tgatccacct ggctcagcct cccaaagtgc tgggattaca ggagtgagcc |
| 226081 | accgtgcctg gctgagactc tgtttttata cattaataat ttactatgga agcagcattt |
| 226141 | atcagtctga ttaaagaaat aagaattggt cattatgggc atcaaatttg tgaatctgac |
| 226201 | tggctttagc tgattgatgg atattatcta tacctattgt ttcagaaaca catattcatt |
| 226261 | gcatgtttat tgtatatatc atagtcttaa gaaaattaat tgaactatgt tttacttaaa |
| 226321 | gggccatata gcagtgtggt taagagagat ctagatttgg agtcaaacac ctgtagctca |
| 226381 | agactgcctg gtttagaagc ctggcttcct tgtttactag ctatgtagcc tatagatcta |
| 226441 | agtctctgta aaatgggaaa acaatagttc ccatttcgta gggatttgat gatcattaaa |
| 226501 | taagttaata catatagatc aaattggtgt ttgaaacagt gattcactaa atgttaccta |
| 226561 | ttattaatca acttttgatt cagtcaacaa ctgtgttaaa gtttttgttt cctgattcat |
| 226621 | agtgggacta ataatactta acctcattaa gttctagtgg cagcataatg agttgatgct |
| 226681 | gataaagttc ttggaacaca taactgacca tcaagatata ctagctatta ttattattat |
| 226741 | tactcatttt gcttccttac actgagagat taagtatgtt aaaattgcta ttcttttcat |
| 226801 | ttatgctctt ttagtcacac taatcaggca tttcttttta gtgaacatca gttttagtta |
| 226861 | acaacatccg gtaggataga accactagat aaaagggata aagagggaag atttcttctt |
| 226921 | ttttgttttt gtgttgagac aggaaaaact ggagttttat gacaacttgt tgtttcattt |
| 226981 | tctctacttt aagatcttca tttgttttgt tactgtgttt tctcctctgc tattgatagg |
| 227041 | tctagtgtgg aagaaacttt gtcttctgga tttcttagtc tcttccacct gtgtctgggt |
| 227101 | ctttcctata atcgttctag ttttaatgaa atctattggc ctgcaattca ttttctttac |
| 227161 | acttaagatt ctgtaaagac tcaacctcca aataaaatag ttgtgaagcc actctcaaat |
| 227221 | ctgagaactg gagtttggaa gttttggact ctaaagtata tttcattatg atgcttaagc |
| 227281 | atgttggagg aggggatact ttctgaaaca tcttaattca cttctgtctt ttgcatttaa |
| 227341 | gtatatttgt accctacacg caagaaattc atttgtcagg gattgtgtgt aggtgctgag |
| 227401 | aggatgggcc gtggcaataa ttagaaaaac aaaagcataa aaagtgagga aggaagaaag |
| 227461 | gaatggtggg ttagtatcct atatgcggga tttcatactg gaagatctta catgcgattc |
| 227521 | cagggaaaaa cttacattga tacctagtga ccaaggcttt cctccagtaa atgagtaact |
| 227581 | gtaatgagta acaaactgta agtccactgc cttcaataaa acatgatgag tgaacctggc |
| 227641 | tgagcagccc tcaggtagtc gttaccagag aatgtagata acggccatga aatggtatga |
| 227701 | agtcactggg agccaagtgg gtgatcagat gtcctaggag aagcagacca gtgcacctgg |
| 227761 | tgacagtgct gggctgggct gaggtagggt ggggtggggt tgtgtatcaa aaccacacac |
| 227821 | ccttagaaac cttatagaac tctgcacccc tcggcaatgt gaaatcaatc caaattctct |
| 227881 | tgtgagcata aagaaaaata ttgacttcaa aatcattgat tttttccaaa atatccattt |
| 227941 | aaagattgac ttcttaagtt tctaaaaaaa aacctgccac tgttttacag gaataagaaa |
| 228001 | gatggtaggt aaagaataga caaaggggcc aggtgcagtg gctcactcct gtaatcccag |
| 228061 | cactttggga ggctgaggcg ggtggatcac gaggtcagga gttgaagact agcctggcca |
| 228121 | acatggtgaa accctgtttc cactaaaatt acaaaaatta gccaggtgtg gtggtgggtg |
| 228181 | cctgtaatcc cagctactca ggaggctgag gcaggagaat tgcttgaaac tgtaaagcag |
| 228241 | aggttacagt gagccaagat tgcgccactg cactccaacc tgggtgacaa gggcaaaact |
| 228301 | ccatctcaaa acaaaacaaa aaaagaatag acaaaggtcc agaaatagag gtgttactgt |
| 228361 | cctgtgaggc cggcctcatc taagtaagta gaggctggaa tttgctaccc atgaaccttc |
| 228421 | cttttccatt gataatgctt ccccactgat aatgtttcaa agccagaagg ctccagaaaa |
| 228481 | tcattggtca atagagatgt ttagctgaat gttagctgaa tgggacagaa cacatgggct |
| 228541 | gatgcagcca gggactgaag gtgtttgttg ttttttcttg ggcaactgac aatgataatt |
| 228601 | tggaagattg ggaaagctag caacttggtg ctgaatctta aatgaccgtc aaagaaactt |
| 228661 | ttgcttttgt aaatctatga aatctgaacc taagacttaa cctcttcatt tttcttcttc |
| 228721 | cactctctgc tttaagaggg tgcttttttt ttctctctct cccaatgaga attaaccaat |
| 228781 | cactttactt ttcacttctg ggggcagggg tgagttgact gaacattctt gcctattgcc |
| 228841 | tattcattcc ccaaagctga agtggttttt taaaagtcat gctcatcacc ccttcctctg |
| 228901 | gccctaatgc ttaacacctg tcaatgactt ctccttacat tttaaataaa attctcactc |
| 228961 | tttaccatgg gttgagaggc cccacacact gtggtttggc ctctccatcc ttatctgtca |
| 229021 | ccactttccc tcattcatgt actccaactg cacaggcctg agttttattc ctcagtagtc |
| 229081 | ccaagccctt gccccttagg atctttgttc tggctttttc ctctgcttga gctgctcttc |
| 229141 | cccagctcag catgttgctg tcttttcttc aaacttcaga tctctgctta gatgacatct |
| 229201 | ctgatgttct ttccttgaca aaactttcta aagctacccc tttcaatact ttcccttccc |
| 229261 | tcctgtagga cctgctatta tctctatctc tatctcctgt catctacatc tatagctaat |
| 229321 | gtctatgtct gtatctctat ctctgtatct ttctatgtat cttacatatt tgttgtatcc |
| 229381 | atacctcact aaaatgtaag ttctattata gcagcacccg tatctatgac attgtgcttc |
| 229441 | acatctaaga taatttctgg cataagatgt gtggagtgaa tgaatgaatt tcagctttta |
| 229501 | taccatccct gtaaaattcc agtaatgggg gaaacagcac agaactgctt gtaagataac |
| 229561 | tatccgtaaa ggatgtggta aactcttttt gagaagaaca ttgtttgcct gagaaaagga |
| 229621 | agagaaaata gcagtaatta taattatagt gatagctaat tactagctag gcactgtagt |
| 229681 | tacctttaca tatatttgct cagttatcag tattaactca agaaataaaa tgcactctgc |
| 229741 | aggtggcaat catggctgct cagcaaaagg cagctttccc ttggatggac tatgatgggg |
| 229801 | taggtcacat gcagtctgca taactgcatt ctacgtacct tctacattcc acatgacagg |
| 229861 | gaatgcttcc atcatgatta acattttctc ttcctgaata gagagaaaag aatgctatag |
| 229921 | cctataaata taaaaaggac caatgcctca caattaatgg attcattttg aaggcaacca |
| 229981 | agacaaagct ctttttgttt gtttgtttgc tttgatctct cttcaaactt tttattttct |
| 230041 | ttgtaatagg tcagtttggc tcctcagtgg cctcatactt cctcttcttg agatggatgt |
| 230101 | atggagtcaa tatggttctc tttatcctga catttagcct catcatgttg ccagaggtga |
| 230161 | gatctgactt ccagtttaca aaacatgctg aaaaatgttt ctcctagaaa agtatttatc |
| 230221 | tcttctttaa agtagaataa ttaactggct attttcacgt cttagagtca taggatttgt |
| 230281 | aatgagatta accttcaaaa tggtgcccat gggccaaagc tcccaatcct tgcactaaag |
| 230341 | ggggtgtaag cctcttgaaa ttatataact tatgtataat ttcacatgta tgtttaaatg |
| 230401 | tgtattttta tgtggaaagt ttcatggctt ctttcagttt cccaaggggg tccctgttcc |
| 230461 | taaatgttaa aaactattgt tcaagaggaa gactttgaca ccttgtgagt tgtatttctt |
| 230521 | tattttgcca agttagcttt gaaagaaatg cttccttaat gcaactcaaa gcctccagat |
| 230581 | tttctatact gaagaaacac aaaatgaaca ttttccccct aagatacaaa gtaagataat |
| 230641 | gcccacaaca cacacacaca cacacacaca cacaaatagt gggatgggat gagagtggga |
| 230701 | tgggatgatg aggtccaaag tcagaaacta aaacacatag agggtccaaa tcaaagaaga |
| 230761 | caaagccctg agctaatgtg gggaaagggt cagctgaggc caaaaagcta gactgaggtc |
| 230821 | agagccttca ctcatatgaa gttaaatgat tctgacacct ttgaatgctt accttacaga |
| 230881 | agttggatta ggttttagtt aacctacaaa ggacctttta ggaccagtat ttcaaagcaa |
| 230941 | atttggtcca tacagcattg acagtggcca aaatttggtg gttttgggtg actttttatc |
| 231001 | ccgatagctt caaatcagtg ccagtttcta tactaagact cctattaaca taataataat |
| 231061 | aataatgctt tacattcata aagagatttg agcttttaat agtacaaagc actttcactt |
| 231121 | cttttactta ctgtgcgtca caacaaattt atgatgtaga ctgtagaaga cattttcttc |
| 231181 | acccatttaa aacatgtaaa tggagtcaca taatgctgat gtgacttagc taacatcttg |
| 231241 | gaatatattt ttttattaag ctgatctagg actagaaccc atgattcaat ccctttctct |
| 231301 | aagattttgc actttaatcc actagtttta taatccttga gataattcca gagcttctaa |
| 231361 | agttgtctgg ctcgtagaag acagtgacca aagatcagag gtcctgtgta gtttatttcc |
| 231421 | caagtgcaca ctttcaggta atgaagctga atgtgtgtat tttatctatt tcaaattaac |
| 231481 | cccagctttt tttttttttt ttttttttgc ttacctttct aaggaaatct gaaactcctc |
| 231541 | cattccatca ctggctacta tgacgttact tagtgtcaga tgcaggtata aatggagttt |
| 231601 | tatcagcagc atttattctt tcccccacac catgctgcct ccatgtggag aaaatgaaag |
| 231661 | gctgttgctg atttatttga ataatacgaa attacagaat gtctgctatg tagtggatgg |
| 231721 | agtgcctacg tgttaggttg gatgtattcc aagagctagg aagcattctg tctttggata |
| 231781 | cccttttata ggttgtatta ggtgctgagg ggcatcctgt tttctgggca atgattacca |
| 231841 | actccagcaa aagaacaaga gaatggacac cctgcatgct gactttgcag aaccaccaga |
| 231901 | ggccagggca gtcatctcct ggcagcaacg ctgtattttt cagctgcctc tacccgacgg |
| 231961 | gctcactcag tattcctcca aggcaaatgc ccttttgccc caaggtcctg tgtccctggg |
| 232021 | caagcctgag accttgatct cctttgtcct ctgtgccccc gaactttggg gcgagacagt |
| 232081 | gtgtctggaa cccaccttct attctgttcg gaccaggaag ccagatgggc caaaggccta |
| 232141 | ctttgggagg ttgtgcagct tgtgcacagg gagtgagcag agattgatgt ccagcccttg |
| 232201 | ttcagtcaag ctctgcaccc aggcttcaag ccatattcat cttctgttcc cagctttagg |
| 232261 | agggaagttt ccatctcaca gaagaagcaa catctgcttg gtaagctatg ggggctagaa |
| 232321 | aggtgctctt tccagttctc accaaggcac cttcctatga ccctattctt gccctctcac |
| 232381 | tggcccactc ttcccagtac tattctccct accgtttttc tcttccaatc tggtagcatg |
| 232441 | aatttgaggc agcagtcctg aattagtgat gaagcagaca ccataatcag ggtgcaagtg |
| 232501 | ttctcacgtt gcagacaccc acaaattaca tgattggatt tccttgagtt ctctctcttg |
| 232561 | acatgaagga cagaatctcc ctccaccctc actcctaagg ggtattctct tatgaaagtg |
| 232621 | tgggtaagat tgttttgttg agtcctatgt gactcaaaag gctacctctc tcctttccag |
| 232681 | acttccaagt cagaacagtt tggtggtggt ggtggcaatg gggtcctcag aaagccctct |
| 232741 | ggggaatgtg gttgacctga ctgcbggtgg tcccctgaac ttggaatgca gggtcctttg |
| 232801 | cactcccttt gttcttacct ctggatttag tagtcgattc ccagaaaatg ggaccccaca |
| 232861 | tggagaccca aagagtcccc ataaaaatgt ttcagaaggg cttttaaaca aaaccatttg |
| 232921 | tgatcacatg tgtggctcag actttgtttc taattgttca ctgctttctt tgcttcttca |
| 232981 | tacagtacct ctggggtttg ccatatggca gtttacctag gaaaaccgtt cccagagccg |
| 233041 | aagaggcatc ggcagcaaac tttggtgtgt tgtacgactt caatgtaagt gtctccacac |
| 233101 | aagtgtattg gtgggaggat ggatttttat ttagtttatt ttattctgaa acccacggcc |
| 233161 | tcctctcctg ggtcaggctg aatgtcttaa gaaagacgtc cctgctccct ttaagaaaga |
| 233221 | aaggaaagaa agaaagaaag agagagagag agagggaggg agggagggag ggagagagag |
| 233281 | agagagagag agagagaaag aaagaaagaa agagaaagaa agaaagaaag aaagaaacat |
| 233341 | gtccctttat ataaatgtga ctatgattta ggacaatggt aatatgatta tatgggaata |
| 233401 | tagactactg gataaaggtg agctcagagt tcaaagactt gaagcaaata ttcttatcac |
| 233461 | tgactttgga aaagaataaa ggaagagttt gcaaatgtgc tttttggaaa atcttgttgg |
| 233521 | attcctaggg aattaaaact tttacacttc ctattaacct ctttacttcc taaacaagcc |
| 233581 | tcctatttag tattactcaa tagttagtac taaataaagc taaatattga aaactaagaa |
| 233641 | atactaaatt ctaactttta aataatacta aaattagtat tattccattg tagtattcaa |
| 233701 | atgagcatta ctccttgagc tcccttaggt agagattatt actatggaaa ggagttattt |
| 233761 | ctgtccattg aaaaaccatt ttggactata tgattcaagt cttcgacaac aatgtaattc |
| 233821 | cacatattct ttttttaagt agtgactaga ctagaaatgt ctggaagcat atgcgtggga |
| 233881 | aagtagccag gttgaaattg aggaattgat aattcaagta atttttggag gctagacaat |
| 233941 | gagaataaaa ttttatacag attgcagaat gaaggaggga aaagcagact gcctgtttat |
| 234001 | agtgttaata tgtatatctc acaaacattt tgatttctac ttttaacctc cttaaattgt |
| 234061 | gagtttccca aagaagagat caggtctgct acatcttttc agcccgtaat ctagtcctca |
| 234121 | tccactacag gctaaatgaa tctcattgac tgaaaatcct gagtgagggg gatgattcat |
| 234181 | agagccttga agtaagaacc acaggaccag tttatgtgag gagtaccaga acatgcaaac |
| 234241 | cttaagaaaa gtttcccatc tccgaatgaa caaagcaagc tattgagtta gcatgaggca |
| 234301 | caatgatagc aacaaatatg tggcatttgt aatcttcaat cacccctgtc agttggactt |
| 234361 | tccatttcaa ttcagcattt tttaaactga attataaact tgcataggtt ttacaaagat |
| 234421 | gaatgtgaca tgattcttga cttcagggta ctcgtgatat tagaaggaag acacgtaagc |
| 234481 | aaccagtata caagatgctg tgcacaaagt tagcaaaatt taaaaaaagg aaaaaagatc |
| 234541 | ctggggaact gagcaagtgg ttcttatttc ttcttaggcg ggtctgggtt ttacagaaga |
| 234601 | gatggcctct gagttggatt ttacctctct tttctatcac ttctagattt tatttctctt |
| 234661 | ttctaccact tctaacttct tccacctctt cctcctcccc tccctttatc cctcccttta |
| 234721 | tttatctctc tcactttcaa atttcaaata atgcattact atgtcccttt accactgttt |
| 234781 | taatttctca ttctcttcct ctgattagca aaatgacaaa cttccagtct gcttaataag |
| 234841 | aacaaggacg aagatcactg actttttctg ttttttaagg catagcagag aaaggaagga |
| 234901 | ctaaaaatat gaacaaaagg tcttttgatt tttatatata gatttctgaa gtttaaatac |
| 234961 | ttctgaattt cccaatgcac tgaaatggaa aattttgggc aaaaatgtcc atttgagatt |
| 235021 | tgaaagttag tgactcagtt gaggaagaca aacagaaaag ttgctgtaag agagttacta |
| 235081 | atgagtggac aagagggtct gagaggcaaa ttcaggggaa agtagaggga aagggtttgg |
| 235141 | atgatgagtg tgcttcccaa gattgtatca ctgcccttga cagctctctc acatccagtt |
| 235201 | tggtgaaata tgatttgtcg gtcagcaggg gatacagaaa ccagagtgtt ctatgatagt |
| 235261 | tatttcaagg cctgcctgaa aacaatacta aggcaatgag agaaatttta aaggtcaaga |
| 235321 | aaatataaga aagccattac actgatttta ttcacttaca ggcttgctgc atgctggtat |
| 235381 | cagtcattaa tttggtatgc atcattgctg gggttagtag tgattttaat agctgtattt |
| 235441 | ctccaatgtc tgcatcatgc agtgaactgt gattggtaag taaggtccct cacttcacaa |
| 235501 | cccacttcat ggtgggtatt tatatctgag actaagctcg agtgttctta tatgtgaaat |
| 235561 | ggagaaaata atagtatcta cctcaaaaag catcaagtga gataatgcat gaaatagcag |
| 235621 | atagctcaga gtatgtagca cacagtacag gctgagtatt ccttatccaa aatgcttgag |
| 235681 | accagaagtg tttcatattt caaatttttt cagattttgg catatttccg tatacataat |
| 235741 | gagatatctt gaagatgaca ttgaagtcta aacacaacat tcatttagtt tcatatacac |
| 235801 | ctgtacatat agcctgaaag tgattttatc caatatttta agtagccttg ttcatgaaac |
| 235861 | aaagatttga tggtgttttg actgtgacct gtcacgtgag gtgaggtgtg gaatttttca |
| 235921 | cttgtggcat catatcggtg ctcgaaacat ttcatatctt ggagcattta agagtttaga |
| 235981 | ttttttttta ttaggaatgc tcaatttcta aatattcctt attacaagaa ccataacttg |
| 236041 | accaaaggca aacagctatt aggaaaccga attgggatgc agggctggtg gtattacaca |
| 236101 | cggtaatgta tgtggatagt ggcaagctag taaaaggact tttattataa caaagaagta |
| 236161 | aatacataag taaataaaag tattctgacc ttaatgagga gttcttgaaa gattcaagga |
| 236221 | aaggaagcaa taggattata tagtcattat caaattgttc attgaataac attattattt |
| 236281 | aaaagaaaat aatactgctt taaggaattt tatatgatat acaagttttc atgacttttg |
| 236341 | aagtttgaaa gatttgtctt ttgttttggc aatgattata ttctaaattc agcaggaaag |
| 236401 | ataggttgat tcatattaag ggacactttg cttttacatt tttgtaagaa aaaggaattc |
| 236461 | agcccatcct acaataagct aataattgag cacattctct atactagggt caaaataatt |
| 236521 | tcacaagtac atcaataaga gagagggaag gacctttaat tattaaactg aagataacaa |
| 236581 | caagaaaaaa atgtacaaag cttaatttaa aatataggag atttggtttc acagtaaaaa |
| 236641 | tccaaaagaa taaagccaag tagtcactgg taataagaca ttatagattc aaatgagatg |
| 236701 | ctgtcttggt caggacgtgg tgtcctccac ggcagaaggg aagaggctgc ctgagcacag |
| 236761 | attgtcttga gtatcgtgca tagatggtag agatcagacg ccacagacag ccgtgggctg |
| 236821 | tccctttaag aactcccata gaatgggcag agatgtccta gaattggaca ataaggaaag |
| 236881 | agtacaagga aggaaaaata aaatctagcc aagctcaagg acctgtatgt gttacctcag |
| 236941 | gttaaccaga cgtttctcag aagacagaat ctaaatttct acttacattg cttttgtttt |
| 237001 | tattatcatt tttgtgaata ataccaaaat taatgtagac taagtactta tatgggcagg |
| 237061 | cactgtgctt agtgctatga tatttctatt tatttatgaa tattatactc atatatttat |
| 237121 | agtgatttat actacttgtt tcagtatgca tcaagaaatg gaaggcacag atatcttgac |
| 237181 | agagataatt taatataaga aattaaacag gtattgggtg actaaaaagg caaaagggga |
| 237241 | cattgaggta acatagaaat ggtaacaggt aacacagacc agtttttacc cctactgttg |
| 237301 | ggagaacaaa agaggaagtg gggttattag aatctagggg cttggaggaa gggcgctgca |
| 237361 | tggccccaag aggtgtgatg agattggttt tgagtgtgct cgaacagact ggaaatggaa |
| 237421 | ccaaataccg ttaccagagt gaagaggcat tgctggagcc atgcattcag gagagagcaa |
| 237481 | atcccttctc ctgacttttt gtctaagtgc ctcctatagg aaaaatctgc aaggaatccg |
| 237541 | atctcaaagg aaaaaagtag ctccccaagt cccgtctcca gcatcacaaa gcagcatata |
| 237601 | caaggatggg tctgaagctg aaacagctta ctaactggca cattattcta tttatattat |
| 237661 | gacctattcc tcttcctagt ccagtaaggt agatatgact attcctaatt tacagatgga |
| 237721 | gagactgagt tttagtagga tttggtattt tcctccaagt tccgtggatg gcaagtggag |
| 237781 | aggcbgggat gctcatctgg tctgcctgac atctctgtct gactctgaag cctaggacaa |
| 237841 | gctgcctcta cataggctac taggagccac tgagatggct acaggtttat actggaaatt |
| 237901 | cacaaaaata agagaatttt agaaatagag gtgctcttcg atatcttcca tcctatatct |
| 237961 | taccttataa agttacagaa attttgagtt actgaagcat gttcagggtc ttatagttgt |
| 238021 | taagaggctc ctaatctagt gttctcttca tgtcctgtat tccgcccgta attttcctgt |
| 238081 | tgtaatttcc agcaacattc tcactgactg ctcctggaac atttactgac tctgtttcat |
| 238141 | gatatatcta tttcacaagg ggaaatggag ttgaagcatt tgggccccat tttaatcttt |
| 238201 | cataataatc ttgtaattcc caaatcagct tctcaaatag aatgagagct ttcatgtttt |
| 238261 | tttttcctaa attctttttg tatcatttaa atattatgct ttattaaaat attcataatg |
| 238321 | gaaaatcata ctttgaaaat tgttattatt aattttgctt tggttggtta gcaaagttct |
| 238381 | atctttagtt tttaaaatta atttattttt gaaatagaca ataaaaatcg tatatttgca |
| 238441 | tgagttcttt gcttgatcct ctgaacaatg acttcataga ggtgctcact ctgtattgtc |
| 238501 | agatgatcta agtatacaac ccagtataca acccagtatt ccatctatca tggcaccatg |
| 238561 | gaggaaatca ttgacaaaat gtttttaaac tccttaaaaa ttcataatcc tgacttttgc |
| 238621 | aaagcccagg ggtgccaaag caggcatccc tgggcttttg aaaaatgcca ttatttaagc |
| 238681 | tagagccgtt atctaggcta tcattatcta ggtagagttt ggggtgtgtg tgacctcaat |
| 238741 | atgaaaacat ttggatccac tctcttggat ccaaagagat ggcatctgga ggctgtctct |
| 238801 | caaaggagat ctgagcagca cacccagcaa caggatcagc tgaccttgga cagactatat |
| 238861 | agaggtaagt tattccacat agaggcaggt gaacagaagc agaatagatt taagatctgc |
| 238921 | acttacagaa taatgtaaga taaacacaag catgatctct tatggtacag gagcctattg |
| 238981 | gatgggaagg atcagaaaat ctttggggta aggtagggga ggttgtgagg aactaccaat |
| 239041 | ctgtagagct tcatctggga accttggaac ctcctctcca tacccatgaa agcgcagtgc |
| 239101 | tctgtgactt agatgtatat atcacaactc atccaaattc tcttcagtta atagtagaca |
| 239161 | aacaccatac acaacatgca tgatcagcta gtctcagggc ttttcaaagt cagaatttac |
| 239221 | catttatgaa acactgtatt tttaaaatat tcttattgag tgcataatga tacatcttac |
| 239281 | tgatggtgct ggaaatctgt tttattaaaa tagtaagaaa cagagagatt attatgtatg |
| 239341 | caatgaatca aataagcaga aattgtatca tgtaaattaa tagaagcaga agaatgagtt |
| 239401 | taagtcagta ggaagaggat gtctttgttg gcaagatacc tggagagaca agggtgatat |
| 239461 | agaaatgata aagaagaaaa ggctacacag ttttaatatt cttgtgagga gaaagaagga |
| 239521 | ggaactaaag tggaatttgg aaggagcttc agctgatatc agacagtgag atcatatggg |
| 239581 | taaaggttta agttgtgagt aaagggaaat gcattctgct tttcaccttg agagcatctt |
| 239641 | gtacattgct gatggataaa tggtatgtct actttgcctc cccttttctg ctgattatca |
| 239701 | tagatgtggg agatagaaga tatcctacaa tgggcaggag aggagactta acacctatta |
| 239761 | tgtgttaagt aggtacatac attaaatttt atcctcataa caactgttta agatggaata |
| 239821 | tatcatccca gattattcct tctgccctgc ctgcctcaca tcccatcttg gaggtttgtc |
| 239881 | cttaaaggag acaggttaga cacatataga atgttaataa aaaggagata agttctttaa |
| 239941 | tcaacatcaa caactctttt cagtaataat gttccaaagt aaaggtgtct tctctcaaac |
| 240001 | atatctcagt tctcttaagt tccttcatct cttattgcac tggacacagg tagaggagat |
| 240061 | attttgtgat tcacacgtgg aataacacat cttttgtttt agaactattt cagtggttcg |
| 240121 | aatgcatctt tatttgaaaa ggtgaatcat tttaggtttc ctgaggtgta gacatcaaaa |
| 240181 | ttatgttaca tgcaggagat attgggggag ggaggcgtgt gaaggataaa ggagagagtg |
| 240241 | aacaagagta gacaggaaga gcttttagat ggtaatgcag gccctatacc tgtgaaagga |
| 240301 | gagagaaaag gaaggattta ggaagagtct cagattgtac tacagttttg aaagagtctt |
| 240361 | ggccaggcta acggtgagtc tctaaacaaa gtatgtccat tagataaatt ccatattggt |
| 240421 | tagcaataac ccagctccag tatttcccat catgctcagt cattatctag gtagtggttg |
| 240481 | gggtttgtgt gacctcaatg tgaaaacagt ttggtccact ctggtgggtc caaggaggtg |
| 240541 | gcatctggag gctctctctc aaaggagagc tgagcagcac gcccagctgc taccttgaaa |
| 240601 | atgtgactgg gctcctccac tttagatcat agctgttcat attgcbgttg ttgctgttat |
| 240661 | ttagtttcaa caggtttcaa atgcagccag aatgacctgt ttcccacatc catcgtctgg |
| 240721 | gcctacctcc gtgcbgggtc agcagaggct gaacttctag atcatgaata attcttcggt |
| 240781 | ggcatgaccc caagcgagac tgctaagcct cttgtggaca aatttagagt tataaatttt |
| 240841 | caaatccacc tagagatacg tctctttccc tagtagcaga gatcataact ttggaggggg |
| 240901 | tgaggaccct ctcatctgtg gatgtagtga gattcctcca atggctttgc tgagcagggt |
| 240961 | gggctgctgc tcaggcatgt aacgcbggga ctggcagtat caaagggctc tgtcccttat |
| 241021 | ttacttcact tcctcaatgt ccagtgtggg tctctttggt tcaacaggct tattaagttt |
| 241081 | gattaaaaga agatgcattt catgttctat tctaggtcct agatcacttc ttttgctttg |
| 241141 | taagcttctt aatttgctac tcctgagtga ctttatgaca cagctctagc ttgtgacgag |
| 241201 | agagattcct gggtctgctt ttagcttcct tggtatttta aacttgggga actgatttct |
| 241261 | taaggtctga agtcctagcg atgccttttt tttttttttt tttttttgct ttagtaacac |
| 241321 | attttagttt tcatcacctc cctcaggcta cacttggcca aaccaaaagt gagcttttct |
| 241381 | tttgcaaata ttagggatgc atatgatcct aatgctatac ttacaatatg aaaacagcat |
| 241441 | tccttctttg gaagtgacca tagaaatgag tcttcttttc attaacatag taaacatttg |
| 241501 | ttgggcattt gccaggtgcc aggcattaag caaagatgag aaaacccaag gtctctgtcc |
| 241561 | tcaggaacat aaagtccgag ggagtagtca aataatcaca aaaataaatc caactgtaat |
| 241621 | tgtgccaagt gctatgaagg aaagctatat agtgttaaga gaacttatag aagacaacct |
| 241681 | gatgtggttt aggagctaaa ggaaggagac agataaaaga aaatattttt ctctgagaaa |
| 241741 | gtaatgttca aattgggctc taaggagtca ggacaacctc tgaatttgaa agaagggaga |
| 241801 | gatattctca gcagtaagaa catcaattac agagaacgtg tgatgataag aggtggcctt |
| 241861 | attcagggga ctgagtaaaa cgagagagct cagaatgtga agggacatca gaacacaagg |
| 241921 | cattggagag acagaaggga accaaagcac gtgggatcct ctaaaatcta gactgcacta |
| 241981 | attatttttt ttttttctca gagcaattaa aagtaactta agtggggggt aggtgggtag |
| 242041 | taacatatca gacttggatt ttgaggtaat caacagactg tagacgggca agatggctgt |
| 242101 | gaggagatca aggaagaatc tttcgatatg actaggttag agactaaggt tggaagagct |
| 242161 | aaatttacct ggcaggaatt ttctatatct aaatattggc tgccaaacta atagattaaa |
| 242221 | ggatgagcca ttgagttagg ttagaacttc acaatgcatt aaattatagt caataattta |
| 242281 | aacacatagc accaaatcat aaatctaata catctgtggt gacaagtaga ttatgtaaat |
| 242341 | ctacattcat ttttttccaa gtagctgcaa tatattcttt acattttaaa ttattttatt |
| 242401 | tgttcaaaaa ctccttttta aatacataat accaaggcat ctcccagtat aggctgtgtc |
| 242461 | cagatgacca cacacgtctc gtttcatgta tgaattcaca aagcttcact ctgaatattt |
| 242521 | taaagaacca gcagcaaatt tataactcca gaagtcaacc agccaacaaa accctctaat |
| 242581 | atgcttttaa tctcctcctt gcgaaatgaa agaagacaac ttttttttct ttgtggaaag |
| 242641 | aaattataca taaaagctca ttttatagca ctgagactgt ctcattttgt taagagtttt |
| 242701 | atgcaattag acagtaggaa aaattaaaga gtttttccag tttcagtgtc tctcagacac |
| 242761 | ttctttggtg ttttgattca ttaaatccca acataagatt attattgcca cgttactgtc |
| 242821 | ttggatgatt tactctattt tattaaaatg ggtagcagag agcagaagaa taaaaaaagt |
| 242881 | tggtgtacat gacaatttaa aggagcttat tctaactgag gattgctaat gattaccaga |
| 242941 | agcaattttt ttttgttcat tcaatcactc cacaaatata aattgaagtc agatgttggg |
| 243001 | aatacaaaga caactaacat atattccctg cccacaggga cattatgatc taattggata |
| 243061 | tgcaaacaaa caattgccat gcaaaatgac aatctgaggc tgatgtacat atgtcttagg |
| 243121 | cacttgggaa cataatggaa taagggttca gtgtccgggt taactggcat tcagatactg |
| 243181 | gactcttgag agagcccagt aaagagaacc tctatttccc atgtgagttc tgaataattt |
| 243241 | gtttaaaaag aagaggcaga tgggcaaaaa atttttccac attcattatt aatggaaatc |
| 243301 | gaattggagg aacaatccgg aattcttagc caaatggtct tgctgattta gaaccccaaa |
| 243361 | ttagccattt tacctctagg gcaacttatt tcctagaaga agaaagatat gtttgggctg |
| 243421 | ggccagccca gttaattctt tcccagttta ccaacagaaa tcactatgcc tttcctggtg |
| 243481 | tgcagtgagt atagggacag agaaaagctg aggcatattg catcaagtga agctccgtgc |
| 243541 | ctctgaagga aatgctgaga gaaaggaagg ggcattcatt acccctatca caactggctg |
| 243601 | tggttgagag gtcaacaaag gctttacaag tatcttgaac cagctttaaa gaaagagtag |
| 243661 | gaatagagca ggctctagaa tggggcaggc aattgggaag cgaattccaa gcagagagat |
| 243721 | cggcatgcac aaaggcatgg aggcatgaga tagtgctatg ctctcaacac ccaagagtga |
| 243781 | ttcagcaaag ttcatattta gggagaaagg agggcagcaa gaggtgagcc tgtcatacta |
| 243841 | aaaaaaaaaa aaaaaattca aaaaagagcc tgatcaggga gagaatttca ttatgctcaa |
| 243901 | gtcagacttt atcctatagg cagcggagcg cacctgagat aggaggaagg agcaggtttg |
| 243961 | gggaattttc ttgaatgtgc tatgttcatg cttcctatga gacatgcaag tggagtgaga |
| 244021 | tgtgtaatgg cattgggcat agggtctaga attcccactg ttagtgtcag tcgtgggcca |
| 244081 | tggcacaaaa ggcaaatgga agtactttct atagaagcca gaagagttaa aaatgcagtg |
| 244141 | tagcataatg acaaaaacta ggcatgagat tccaacaaac ctgggtttga atcctgccac |
| 244201 | atacgaccta tgagatatta gacaatcatc ctgaaccttg tagttgccat gtataaaaca |
| 244261 | gcagtattaa tacctagcta ccaggcttgt ataaaaaata catgagatta tgtgtgtaaa |
| 244321 | gtattagcac atctcggctg ggcgtggtgg ctcacacctg taatcccagc actttgggag |
| 244381 | gccgaggtgg gtggatcaca aggtcaggag atcgagacca tcctggctaa catggtgaaa |
| 244441 | ccccgtctct actaaaaata caaaaaatta gcbgggcgtg gtggcgggtg cctgtagtcc |
| 244501 | cagctactcg ggaggctgag tcagaagaat ggcatgaacc caggaggcgg agcttgcagt |
| 244561 | gagccgaaat tgcgccactg cactccagcc tgggtgacac ggtgagactc tgtctcaaaa |
| 244621 | aaaaaaaaaa aaaagtatta gcatggctct ggcacatagt agattcttct ttaaatgcct |
| 244681 | cttcaggata cttagctcat gaaaattccc atttgttgga gggacaagga aacagaagga |
| 244741 | aggaacttgt agccagagca agaaagtgga agtggggcag cagccatgtc ccctgtgtag |
| 244801 | gcaggaggca gggggacaag agtaccaatg caaggatgga gggatggata cccaaaggaa |
| 244861 | ctgcctcatt cacagctgct tgaggggcag agatcactgt ctacagtcct taacacaact |
| 244921 | tgattccctc tgctgaaaat tagatctgcc taggctcagc tcagcttcct ctttgtactc |
| 244981 | cacactgtct ctgttaagaa ccatagagca cctgtttcat gtctatgaat ctgggttgga |
| 245041 | gcaaaaggaa aataaaataa aaaaaactca gagatgagca aagtattagg cagaagccta |
| 245101 | atgggtctgg ctggagagga ggagtgtggc catgcgctgc tgtcagcatg cagctcacaa |
| 245161 | ggcccgggct ggctgctgtt cttgtgacgt tagttgaggt ttattctggg cataattagc |
| 245221 | cttttcactc cttgtttctt tcctgtgttg cgtcctactc catgaaatgt tgagtgccag |
| 245281 | tccttgtctt tcatccacac ttgaaacctt cgttcatgct aaggtgtaaa taaccagcaa |
| 245341 | caactcagag aaaagtagaa atacacccag acttgaatct tgcagtgtca cctcccctca |
| 245401 | cataccctca gcagatatgc ttaaaaaatt atatcttcct ttcctaccag ttttacttta |
| 245461 | tacattcttt caaaataatg gtctggcagc tattacaaat taaaaagagt tactaagaat |
| 245521 | gaagaaattc aatgatcatc ctggggatag agtgttatct ttcatggtga acatttcttg |
| 245581 | gtaatcatga gaaacaccaa agggatgcag ttttaatata atttcaggca acaaataatt |
| 245641 | atgaaggaga tgttttgcat gcagagcctt ttggggacct ctgaaatcct gtctcctaat |
| 245701 | ccatctatgt atattgggca aatagtaagt tctcaataag tgttagttag aaaagtaaat |
| 245761 | gcatgaattg agtcctggta accataataa aaacagtctt tcttgtggaa atgtgttggt |
| 245821 | gattatggac tggaccagct gcagcaatca aacagaaatt agagtcttaa tagacaacac |
| 245881 | acgagcatgg caccccagag ttggacagaa attttctgtg ctcactggga acttggctct |
| 245941 | gactctggtt tctctgtcac agattttggg gtgggagagt gctcccatgg ggaaagcagc |
| 246001 | acgtgctgat atcgaaattg aatagaacct tggctgggct aggagtcatt gctattttag |
| 246061 | tcacttccag atgtctgctg ctgtagtttt aagggacagg gaaagatgag tggagtgtgg |
| 246121 | catgccactg ggtaaaagat gaggagcgtt ctccttctaa gcccttcaag ttgtatttaa |
| 246181 | agccatccct caatatgcta caaagatgga ttggataaag cctttaaaaa ctgtgttctt |
| 246241 | ctattcacaa tagcaaagac ttggaaccaa cccaaatgcc catcaatgat agactggata |
| 246301 | aagaaaatgt ggcacatata caccatggca tactatgtag ccataaaaaa ggatgggttc |
| 246361 | atgtcctttg cagggacatg gatgaagctg gaaaccatca ttctcagcaa actaacacag |
| 246421 | gaacagaaaa ccaaacacca catgttctca ctcataagtg ggagttgaac aatgagaacg |
| 246481 | catggacaca aggaagggaa catcacacac tggggcctgt tggggggttg ggggctaggg |
| 246541 | gagggatagc attagaagga atacctaatg tagtgacagg ttgatgggtg cagcaaacca |
| 246601 | ccatggcata tgtataccta tgtaacaaac ctgcacattc tgcacatgta ccccagaact |
| 246661 | taaagtataa taaaaagtta attaattaaa aataaataaa caaaaactgt gctttttaac |
| 246721 | attcatttat agatttgact tcatatatat caaataggca aactgcagaa acctttgtga |
| 246781 | actaatcatg actctgcctg ccccaactag aaaaacaaaa caacacaaaa caaaacaaaa |
| 246841 | ccagaaccat tcttgggctt tggttatgat ctagtattta tgaagtgcca gccattaaac |
| 246901 | atatgttgaa tggtacctat gccagatgag ctggctgccc ccagtagcta ttgtgctaac |
| 246961 | ctaacaagct caccagatgc ttgttctata tctgctttct ccttttatga tattttcggt |
| 247021 | gtgtctttta atcccaaaat tgtcattatg aattagataa aagatatcag gtggggggtg |
| 247081 | accccctctg aaatattctc aaaagggatg cctggggttc tttccaagag gccctgtcac |
| 247141 | caccagggac cagtctaggg tatagatcaa agtctgtgat gggttcatag agtttcttgt |
| 247201 | ttctttcctt tctttcactg agaaggtatg gcactagcat gatataactc ttgctggata |
| 247261 | aagaaagcac aggagaagca gtatagtgca gtggttaagc atataggatt tggattagac |
| 247321 | tcaggtcaag ttcagacact gtcactcatt agtcatgtga acttggacaa attacttaac |
| 247381 | ctctctaagg ctcagtttct tcatctgtaa aataggtata gtgatagcag ctattgaatt |
| 247441 | tttgtgtgtg caggagaggg gaaaattatg gtaaataata attcctccct tgtccaacaa |
| 247501 | gattgtgagc tcaccggaaa tataacaaag ggtaagacca gtgaaggtca acatgccgcc |
| 247561 | tttattattg ttaaagctga cataaaagaa tgtagtgtct ttatggacaa gtgaacttca |
| 247621 | cagccagacc tctctgccag gacaggcctc tccttgcagg agcagagcac agcatgttcc |
| 247681 | ctctcaagag aaggaagaga tgtccgcatg tctccatcca tgctctactg tcaaatcagt |
| 247741 | cacttttccc cgaggctaga gtgaggcagt ggagggtggc tgggagtgtt atgttaatgg |
| 247801 | gtcttcactt ggaagggaac gtcaagagtt ggagatacga ttaccctagt agcagcaact |
| 247861 | tcatagtgtt ctcctgagga ttctgtgaga taaaataagg tgtttggcaa gtgctaaatg |
| 247921 | ttcacgaaat gtatacggtg atgttacttg tcacagcaac cattatcact gagtgtcaca |
| 247981 | tctgtggcaa ggaaataggg caatgaaaaa cagagacatt tggaagttgt ggtgttgtcc |
| 248041 | catgagggtt aaataaagag ttcaggatat aatctttaaa ccaaacaaga gattaagtgt |
| 248101 | ctatacttag catgttctgt tattgtcacc ctgggacaga ggaacaagca tgaaggttta |
| 248161 | agacatgata gaaggagatt ggatttaaca caagtgggaa gaagcagaga tcttaactag |
| 248221 | ataaatgtga aacaagaaga tgtcatgcac ctgggagctg ggagatcctc ccaagcatat |
| 248281 | tctcctggga tgaagatttc tgggtcttgg ggagttggtc aatccatgtc ttatcttaga |
| 248341 | catgagatta gctcacccac tccctctgct tgaggtttct actagctgtt tatccccaaa |
| 248401 | tgttctgttg gatggcatcc tagtcagggt tctccagaga aatataacca gtagaagata |
| 248461 | tgtatacaca catacacaca tatacatata tatatatata aaatttatta tatcaatcta |
| 248521 | gtatttatat aataaattta ttttgattta tcattattat tacttaaatt tattatataa |
| 248581 | atctttatat atatatatat gcatataaaa tttgttgggt tttttttttt ttttttgaga |
| 248641 | cggggtctca ctttgtcacc taggctgcag tgcaatggtg tgatcttggg acactgcaac |
| 248701 | ctccacctcc tgggttctag caattctcct gcctcagcct cccaaagtgc tagaattaca |
| 248761 | ggtgtgagtc accatgtctg gctgatatat ataatttata agttaatata aataatagat |
| 248821 | ttactacaga tataaaagga gattcatttt gaggaatagg ctcacacaac catggaggct |
| 248881 | gagaaatctt ctaacctgac atctgcaagc tggagatcca gagaaaccag tgctgtaatt |
| 248941 | cagtctgaat ctgaaggcct gagaaccagg ggagccaatg gtgtgaatcc cactctgagg |
| 249001 | gcaggaggag atgagatgtc ccagctcaag cagtgagtga ggcaggagaa aaaggagcaa |
| 249061 | atccctcctt cttccttttg ttcttcctct ttttgtcctc agtggattaa atgatgtcca |
| 249121 | gccatgctgg ggagggcaat ctactttact gagtccacct atttaactgc tcatcttatc |
| 249181 | cagggacacc ctcccgggca tacccagaaa cagtgtttga tatggacacc ccatggccca |
| 249241 | ctcaagttgg cacatacaat taaacatcac aagttggctt tattttttct taaactgaaa |
| 249301 | aatgcagata caggcctgtg tggcccaggg aaacccagac ctttggtatt tacctgtgat |
| 249361 | tcattccgtg cagtcccgag ttaagcacac aagttcctct gagattattt ggagatttta |
| 249421 | ttttgttcta gttcttaaag agtacaatat ttaagaaaat ttgccccata ttttgaaagc |
| 249481 | aatagttttt cagatctctg ttggcctctg atttgagtta ggcatgatgt ttgtttaagg |
| 249541 | caattttcta aattacttct cttaattctt cctctaggaa ttagcatcta cgacatttct |
| 249601 | actttctttt ccacacctcc actctccccc acttcacttc ccttcttcag ctgtgagtgt |
| 249661 | caaagtccct gggttcgtat aaatgatgtc aaaggcaacc aattgggttt tctaagattc |
| 249721 | agggttgact cacctgatct caagaggaag ccccatattg acaagtgatt atcttctcat |
| 249781 | ttcacaaaac atagttattg tcagagagaa gtaagagggg agcbggggag tggatagctg |
| 249841 | tagtgataca gtaacagtgc ttcaatgctg aaagtgaaaa acacagtgaa tgtgaggaag |
| 249901 | agaaaaggtg catattataa aggaagcagc atcagaatat gccaatgttc tgagaataaa |
| 249961 | gagggagtta ttcaggtggc tttgagagag ggtgatttca accctggcag ataattttta |
| 250021 | ggaacgagaa tctaatgcac aagtaaacat gcttgttata tgggaatgct gtgatagtct |
| 250081 | tgatgaatgg gaagtgtgca gatgagttcc aatgtcatcc cacctgctct ttgatatttg |
| 250141 | tgaagtgaga gctgagacat aacctctgtg gtttaagaag aactgatcta attatggtca |
| 250201 | ggtgattgga aatggaaatc tgccacagca ctttgaggaa gcatttttca gatccccatc |
| 250261 | ccctagtatt aagcaaactc ttaaaatcgt aaatctaatg ataccacgaa atgtgttttg |
| 250321 | atgtttaaat caaagggcat ttcacggtaa tggctccaca gctgagcaag cctgtatttg |
| 250381 | cttaaaaatt ttatctattt agaagtcact gattttcaaa taaactgaaa agctgccaat |
| 250441 | caacatggca gctgaaacat tcactttatg gagcaaaaca atagggctca tgtcagagct |
| 250501 | gtgacccaag tttgaaaatt aatgtcattt tgttatttct cttaagagtt gatcttttcc |
| 250561 | tttgagttgc tcttcacgac aactgctaag tggctttgtt gttggatttc agggtttggc |
| 250621 | acaatattcc gttctctttt atggctatta tgacaataaa cgaacaattg gatggatgaa |
| 250681 | tttcaggttg ccgctctcct attttctagt ggggattatg tgcattggat acagctttct |
| 250741 | ggttgtcctc aaagcgtaag tttcatttgt cttttgggaa gcaaataatg atttctggaa |
| 250801 | tcaaatggag ggattaattt ggggattgga tataattttg ttgctatttt atgtctaaag |
| 250861 | gaaacagagt ctgtaagaga tgaaatgtag ttttaatacc aacactaatg ttagactatt |
| 250921 | acatcttcaa aagaactgaa cataaacaca ggcctttgag tcatctcatg gggtcagacc |
| 250981 | aatgcttgca tgcctatctt gtgtttaaac atatttttta aatgtgtatt tttgtacttg |
| 251041 | aaggtgtgaa ttaatgttgc cagcaagaca ggaaaacagc tgcbggtctc tcttttgcgt |
| 251101 | gcagagaggt ttgtaacttg cagaatcagc tcaataggca gaatctattg ttctctgctc |
| 251161 | attttttaaa aaaaccagaa taatcactca ttcaaaaaat tttagctgtg taagacagca |
| 251221 | aaaatgaatg gtccccaatg aaaccatcac aatcaactgt ataatcatgg tttgtcttgg |
| 251281 | gaaaagattt attggagtat ggggaagaat ttcactggag aactgatatt actgcagttc |
| 251341 | atttttctcc ctgcctaaaa ttatttataa tactccactt aaagactgca gaagcctagc |
| 251401 | agagttattt gggtttatat attaaaaaaa tgacccttta gccaattttt tttgaactag |
| 251461 | tttacgagaa gagttgaaac atctcaagta aggttgccat tcttttattt gactatgtgc |
| 251521 | aagttgaatg agcttttccc ctcattgtag gaggtccctc agcctgaggg tctaagaggg |
| 251581 | gaaaagtgca aagtcaccct aagcaggagc tctctgaagc agtctgtgac acactttgtg |
| 251641 | tcctaaacct gcctcactta cttgttcctc cagagaaaac tgccatattt catttttaag |
| 251701 | ctcttttagg acttaaaacc ttataggaaa tcctatatct aagatattta tcaacctgct |
| 251761 | aaagtgtaat cagagacctc aaatataaga gactgtccat cagataaact actatcacat |
| 251821 | tccaaaaatc gaagatattt tctagtcttt tgactatatt gctctggttg ggctgaagtc |
| 251881 | agaggtatca tttacattac tgaggcctca tgagtatcat ttctgtgttg ctcctttagt |
| 251941 | ttctgtggtc agtggagaag aatagtttct catgtctgat gactcagctt ttattccggt |
| 252001 | aactgtgcag gctgtgtacc ccacagccat tctgcctagg ttcaaatccc agctctacca |
| 252061 | tgtatgtatt tatacaatca ttaacagatg gctcaactgc tttgttctcc tgtttcccct |
| 252121 | tggtaataga ggtgtaatgg atgtgaggac taaatgaatt aatacaaaca aagcacttag |
| 252181 | aatatgcctg gatgagaaaa ttattcaaca aatgttaaca attttattag gctatatttt |
| 252241 | attaggctat attatatttt attagcctat aattcatagg cttatttaag aaaaaaagcc |
| 252301 | agcatggtag gagttgaggc cacattatag atccccaaaa caaggcagtc atgtatggtt |
| 252361 | atgcagttgt acatcagtag atttgtatat ttaataaatg attttctggc agatgaattt |
| 252421 | aaagtatcta gagcagcatt gtgccaggca agccacatat ataagtttaa aacttctagt |
| 252481 | agtcattaaa taaatagaca ggtacattta attttaataa taaatatatt tgctttaact |
| 252541 | caatatatca aaagtattat cattttaacg aacaatcaat ataaaattat tattgagata |
| 252601 | ttctctgcct ttcgaaaggt atcaagtctt tgaaatctgg tgtgtatttt ataccagtcg |
| 252661 | catttcaatt gttcagtagc cagatgttgc tggtggaaca ctctgttggg tagagtggct |
| 252721 | ctagaggaag tagtggcttt ttcaaagttg ctcgaagatg ccatttggga tagctgcagg |
| 252781 | gcatcaaatc cacattttcc ccctcaaagt cattcatgtt atatcatcca aaccagtgtg |
| 252841 | gccttgaacc ttccctctgg catggacctt tggctgctcc ttacctggtt aacaagtagt |
| 252901 | tcaaactcat tgcccaaaca gcaagacaac tcaaaaacaa tacaccactc tcactccacc |
| 252961 | accagcccat gggagaatat tcagtagaaa aagaattgat gagatataaa tctttgtaca |
| 253021 | gaatcattaa tcatctcttc ctactcctat cccagtcaaa tattctcttg gaataagttt |
| 253081 | gagcggattt atgacataca ttactcaaat ggaatagaag gtaacctaag ttctaaaatc |
| 253141 | gactaggcta catcaagatt ttttcactgt actcattctg ttagcgtctg catactcatc |
| 253201 | cacaaaatgg gagcaaatat ctatttttag ttacttaata gcaaaatcag aaaataaaaa |
| 253261 | gatagaaatc caagaaaata gtttgaaatt ttaagataag atgactaact ttgttggccc |
| 253321 | aggattctcc tggttttaga actgaaagtc ttgcatttga gaacctttca gtccccaaca |
| 253381 | aacatggatg gttggtcacc ttggttttaa gtccttccct tttcttcttc ctcccttcat |
| 253441 | tcatttcttt tatccttttt ttttttcctt ttctttccag gtcagtgtac attttcaacc |
| 253501 | acatcatatg cttagaaatc ttcatgataa atatagttac ataagtatta aaagcaaatt |
| 253561 | aaaacagtat aacataaagt tacacaacat ggaaaccatc agctcattta aatattgtcc |
| 253621 | tcattagttt gaagttttat atcataaaat aaaggtggct tgacatcaga acccaactgt |
| 253681 | tcccgttaaa aacaattctc taagctaaaa atttggggtt ttactggtct attttacagc |
| 253741 | agctaaacta ctggaagcag ttgaatcatt ttgagaatgc atgtcttaat aagaaactca |
| 253801 | tagctatttt atccttgtag gctttttcaa gactttttcc tgtttgtagg atgttttagt |
| 253861 | ccatttcgtg ttgctataaa ggaatacctg aggcttggta atttgtaaag aaaagaggtt |
| 253921 | tatttggctc acagtctaca gactgtacaa gaagcatgga gtcagcttct gcatcaagtg |
| 253981 | agtgattcag ctgtttccac tcctggtgga aggcaaaggg gaaccagcat gtgcagagat |
| 254041 | cacgatggtg agataggaag caaaagagag atggggaggt gcbgggctct tttaaacagc |
| 254101 | cagctctcgt gggagctaat agaataagaa ctcacttact atcccctgca aaagaaggca |
| 254161 | ttaatctgtt cataaagaat cttcccctat gacccaaaca cctttcatta ggctctacct |
| 254221 | ccaacattga gatcaaatct ctacatgaca tttggggaga caaacagcga aaactcagca |
| 254281 | cagggctccc tgggattaga tataattttt ctgtttatga tcctggctat aataatactc |
| 254341 | atagaatgga ctaacaaata ttcttggata cttaaatcat gtgctatacg taacccttaa |
| 254401 | atggtgtcaa aatttttgtc tacatagact ataatataga ttatatatat atgagatata |
| 254461 | tattataaac tacatatatc acaaaccaac ctcaacacat gcagataaat atttttaaat |
| 254521 | ttggcaaaat gacagtatgt tcatccatat ttctggagct gcaacaataa ttataaatta |
| 254581 | tacttgaaat aataataatt aacatctgtt gagcatctct catgaatcag tttaaattac |
| 254641 | attacttcta atccctaaac attgcaaagt agctattagt tttataattt tacagataat |
| 254701 | gaaataaaac tcagagatcc taaatgaact gcacaaggtc atttgactat aagacctctt |
| 254761 | tgaatttaga gttttctagt tcaaaagata tcttctaccc cttacacctt gccacctctc |
| 254821 | tgtactgtgc ttctccttac gtaggccaag gtgttcctcc caccaatctt atatactgtg |
| 254881 | ttgagtaccc attagttatt cctattggat ggatggagaa attaaggctc agcagagttt |
| 254941 | gaggatttca ctaagttcaa atgcccagta atttgcataa ctgaactttg aacaccttgc |
| 255001 | tttaatttca gcacttagct tttctaattt ctgatgtgga ttcatctata ttgatgataa |
| 255061 | caatcatcaa tatagactga tgtctctgtc tcgtcctggg tagccttttt atttttaaaa |
| 255121 | attctgtatt aaaatagttt cttgtctgtt gttaaattcc cttagagttt tatggccttg |
| 255181 | atgtgactca tgtgaccaaa cgttttgtta aaaaaaaaat taaacttatc aaaaccaagt |
| 255241 | ccttgttcta caagactctt gcatattcca agataaatca tggccagctt cacacacacc |
| 255301 | atttctaaat caacgagact ttattttctt ccttctactt agatgtcagt gcctttgtcc |
| 255361 | attttctttt cacttctttg gtacatgttt acccagacag ttatgaccta cctgatactc |
| 255421 | tgcctaaata tcttccatgt ctcttgacat ccttaatttc ttaacattga aattgtttct |
| 255481 | ttttcctccc cattttccca cccccaatta atttttttct ttgtcgaagg aatttctcct |
| 255541 | tatctattta gtagaaagca gtcagagtgc ctcatagacc ccagtctaat attttacttc |
| 255601 | tcagagatat tttcattgga gatcataatg tttgatgtat gtaatgatga atttcagacc |
| 255661 | ctagagaaag ttatggaagg gtaagtggac ctggcctaca gggcatgata ttctggcaaa |
| 255721 | tcccacaaaa catatggtta tctcatcttc ctcaaactat agttgacctt atttcaaaaa |
| 255781 | tgcttggagc agaaaagcaa cgaggtatga aagacatgaa gtcaccacct tccattttat |
| 255841 | attactactc tatgaattgc atctagattt taaaactcaa cataaaaaac tgtcttcttg |
| 255901 | caagcatgga ctaataagga tagtagtagt tcacaaagaa tggcagctat ttacaaagtg |
| 255961 | aggtgatatt gtcaaaacct atcataatga caaagacaga actaaacagt gatagaataa |
| 256021 | caagcatatg tggtatgcat ttattatatg tattgtgcca tgcaaaaaag caatttatgc |
| 256081 | tgtatgtatt tacttggtga ggctctttcc agtgctaagt ttacttctag aatgacaatt |
| 256141 | catgtattca ttcatttgat tactatttgt ggaacaccta gaatgggtaa tctaagagaa |
| 256201 | ctatccagta cctgggtatt tcatggtgag caaaataaag tccttgcctc tgtggtgctc |
| 256261 | agttggggaa atattgtaaa caaagaaatg accagtggtt ttggccaatg tagtataatt |
| 256321 | ttatgttgtt gttatcctct gcagtcaaat atttgctaca gcttcttcat cctcaagaat |
| 256381 | tttaaatagt tgagaagagc caagatggct aacaagatgc agccaggaag agcttctccc |
| 256441 | aatgagaaac cagggtatta agaagattgg cacactccaa gtagatcttc agaagggagg |
| 256501 | cattgagagt ggatggagga aggatgtaga ctctgtgctg aagttagagg aagcttggaa |
| 256561 | tgctatgctc aggaatgagt tctaggactc attcctggct ccgagtgtct cctaaggaag |
| 256621 | ggagagttaa ataggcgtgg agtagcccac tctcaccatg gacctccata attctagctg |
| 256681 | caggagtctc aatgaccccc atggatattt gaactgatag ggggagctgc ttgaagagtt |
| 256741 | gacagggaca ggactgcaga ctgtgtggag ttcagaggat ttggcaaagg aaggactgca |
| 256801 | gtagagcaca gccaaggaca cccatccctg aagactcacc atgcccctgt aggctttggc |
| 256861 | ctttgttgac tgtcagacca atacagagca gggctgtctt gcccatggaa tgaggccatt |
| 256921 | ctgatctgag tgcctccctt gtctgccagc ctttcctgag gttcatgcca ggctgtgccc |
| 256981 | acttgcagca caggcttgga tgtccaactg gggcacttcc taaaggccat catcatagct |
| 257041 | tctttgttgg cagaccccac ccaactgttg gagagcttct gaagacaagc ctccacccgc |
| 257101 | acacaaccac ctgcagcctc ccccctctgc tttgccaaca cgcaggtatg catggaccct |
| 257161 | gctgctctgc tgccaccaac acatgagcac agaccccact gccactgccc cgacaaagca |
| 257221 | ctgttgccag caaccctcat ggagcattgt tgttaacaga ctggaaacac ctcagctcct |
| 257281 | tcagcacagc aggtgtttaa ccttgagagg ccagagaaca aagcbgtgtg cctggtccta |
| 257341 | taccactggg ttacagcaca cagtccagga gtgctgagct gagcccaggt cccctgaaat |
| 257401 | cagccagaaa tgaagccagt tgactagacc taacttatat cacagtcaaa acctcaaagg |
| 257461 | catcaaagaa ttgcaacttt aaagattaaa gaaacatcag ctcacacaga tgagaaaaaa |
| 257521 | ccagcaaaag aactggggca actcaaaaag ccggagtgtg ttctcacttc caaatgacca |
| 257581 | tgctagctcc ccagcaatgg ttcttaatca gactgagatg gctgaaatga cagacataga |
| 257641 | attcagagtc tggatggcaa tgaagattgt tgagatttag taggaagtta aaacccaata |
| 257701 | cgatgaacct aagaatccag taaaacaata caagagctga aagacaaaat agtcatttta |
| 257761 | agaaccaagc tgatctgata gagctgaaaa ccccattaca ataatttcat aatacaattg |
| 257821 | gaagtatcag gagcagaaca gcccaagctg aggaaagaat ctcagagctc aaaagtggtt |
| 257881 | cttcaaattg actcagtcag acaagaataa aaaagaaaga ataaaaagga atgaacaaag |
| 257941 | cctccaagaa atacagaatt atgtaaaaag accaatccta tgactcactg acatccctga |
| 258001 | aagagagggg gagagagaaa gcaagttaga caacatattt gagggtactg cctatgaaaa |
| 258061 | tttccccatc ctcactagag aggtcaacat tcagattcag aaaattcaga gaacccctgc |
| 258121 | aagatcctat aaaagatgac catccccaag acacatagcc cacagattct ttaaggtcaa |
| 258181 | tgcaaaagaa aaaatattaa aggcagctgg agagaaggga aagacatcta taaaaagaac |
| 258241 | ccatcacact aatggcagac ctttcagcag aagcagtaca accaagaaga gattagggcc |
| 258301 | tatattcacc atcctaaaag aaaagaaatt tcaactaaga atttcatatc gagcctaact |
| 258361 | aagcttcata agcaaaggag aaataaaatc cttttcagac atgcaaatgc taaaggaatt |
| 258421 | cattactatc acacttgcct tacaagaggt ccttaaggaa gtgctaacat ggaaataaaa |
| 258481 | aactgttacc aactaccaca aaaacacaca taagtacata gaccactgac actataaagc |
| 258541 | aactacacaa tcaagtccac ataacaacca actaacaaca ctatgacagg atcaaatctt |
| 258601 | cacatatgaa tattaacctt aaatgtaaac gggctaaatg ccacattcaa aaggcacaga |
| 258661 | atggcaagtt ggatcaagaa gcaagaacca actgtatgtt gtcttcaagg gacccatctc |
| 258721 | acatgcagtg acacccacag gctcaaaata aagagattga taaaaatcca tcaagccata |
| 258781 | aagagattga taaaaatcta tcaagccata aagaaattga taaaaatcta tcaagcaaac |
| 258841 | agaaaacaaa aagagaagag cctgttattt ttattttaaa ccaaacagac tttaacctaa |
| 258901 | caataatcaa aaagggacaa agaagaggat tacataaaga taaaagattc aattcagcaa |
| 258961 | gaagacttaa ctgtcctaaa tgtatatgta cccaacactg gaacacccag gttcataaaa |
| 259021 | taagttcttg acccacaaaa agacttagat aactacacaa taatagcaag agagttctgc |
| 259081 | accttactga cagtattaga cagattattg aggcagaaaa ctaacaaagg tatttgggac |
| 259141 | ctgaactcaa cacatgacca aatggaccta acagacatct acagaacact ccacccaaca |
| 259201 | acaacagaat atacattctt cttatctgca aatgacacaa actataaaat caaccacatg |
| 259261 | ctcactcata aagcaattct cagcaaatta agaaaaactg aagtcacacc aacaacattc |
| 259321 | ttggatcaca gtgcaataaa aatagaaatc aataccaaga ggatctctaa aaatcacatg |
| 259381 | attacatgga aattaaatag cctgctcctg aatgactttt gggtaaacaa tgaaattaag |
| 259441 | gcaaaaatta agaaattatt tgaaactagt gagaacaaag acatgacata ccagaatctt |
| 259501 | tgggacccag ttaaagcagt ttaaaaggga agttatagca ctaaacaccc acatcaaaaa |
| 259561 | gttagaaaga tctcaaatta acaacttaac atcacatgta gagaaaccag agaaacaaga |
| 259621 | gcacaccaac cccaaagcta gcagaagata agaaataacc aaaatcagac ctgaactgaa |
| 259681 | tgaaattgag acacaaaaag ccatacaaaa ggtacataaa tccaagagtt gtctatttga |
| 259741 | aagtataaat aagattgata gaccactagc tagattaata aagaaaaaag aagattcaaa |
| 259801 | taaacacaat cagaaatgac aaaagggaca ttaccaacaa ctcaaaagaa acacaaaaaa |
| 259861 | ccctcagaga ttattacaaa cacctctatg cacacaaact agaacactta gaagaaatgg |
| 259921 | ataaattcct gaaaacatat aacctcccaa gattgaacca ggaagaaatt gaaaccctga |
| 259981 | acagaccaat aacaaattct agaattgaat cagtaataaa aagcctatca accagaaaaa |
| 260041 | gtcctggacc agacaaattc acagccaaat tctaccagat gtataaagaa gagttggtgc |
| 260101 | caatcctact gaaactattt caaaaacttg aggaggaggg atgcctcttt aactcattcc |
| 260161 | atgagaccag catcattctg ataccaaatc ctggcagaga cacaatgaga aaataaaact |
| 260221 | tcaggccaat atccttgata aacatacatg caaaaatcct caacaaaata atagcaaacc |
| 260281 | aaatccacca gcactcccaa aagtaattta ccatgatcaa gtaggcttta ttcctgggat |
| 260341 | gcaaggttgg ttcaacatac acaaataaat aaatgtgatt tatcacataa acagaatgaa |
| 260401 | aaacagaaac tacgtgatca tctcaataga cgcagaaaag gctttggata aaattcaaca |
| 260461 | tcctttcatg ttaaaaactc tgaacaaact aggcattgaa ggaacatacc tcaaaataat |
| 260521 | gagccatcta tgttaaactc acagccaaca tcatactgaa caggcaaaag ctggaagcat |
| 260581 | tatccttgag aactggaaca agacaaggat acccaccctc actattctta ttcaacatag |
| 260641 | tacgggaagt cctagccaga gcaattaggc aagagaaaga aataaaaggc acgcaaatag |
| 260701 | gaagagagga agtcaaacta tctctctttg tggacaatat aattctaaac ctagaaaacc |
| 260761 | acatagtctc tgcccaaagg ttcctagatc tgataaataa cttctgcaaa gtttcagaat |
| 260821 | aaaaaaccaa tgtacatttc tatacagcaa tattgtccaa gccaaaccag gaatgcaatt |
| 260881 | tcattcataa tagccacaaa aagaatataa tatctaggaa tatagctaac cagggaggtg |
| 260941 | aaagatctct acaatgagaa ttacagaaca tagcttaaag aaatcagaaa tgacacaaac |
| 261001 | aaatggaaaa attccatgct catagatggg aagaattaat attgttaaaa tggccatatt |
| 261061 | acccaaaaca ctttacagat tcaatgctat tcctatcaaa atacaaataa cagtttttag |
| 261121 | agttagaaga aactatttaa cattcatatg gaacccaaac accctgaata tccaaagcta |
| 261181 | tcacaagcta aaaattaaaa aaaatttaaa aagctggagg catcagaatc acactacctg |
| 261241 | acttcagact atgctacaag gctacagtaa ctaaaatctc atgatactga tagaaaaaca |
| 261301 | ggcacataga ccgtattagt cagggctctc tagagagaca gaactaatag gagatatata |
| 261361 | tatatatatg ggagcttatt agggagaaag gctcacatga tcacaaggtg aagtttcgtg |
| 261421 | ttaggccgtc tgcaagctga ggaaggaaga agccaagagt ggctcagtct gagtccaaaa |
| 261481 | gcctctaaaa cagggaaacc gacagtgtag ccttcaatct gtggctgaag gcctgagagc |
| 261541 | ccctggcaaa ccactggtgt aagtcccaga gtccagatgc tgaagaacct ggagtctgat |
| 261601 | atccaagagc aggaggatga aaggaagcat ctagtacagc aaaaagatga aagccagaag |
| 261661 | actcagcaag ccagcttacc ccactttctt ccacctcctt tgttttagcc actgtggcag |
| 261721 | ctgattagat gatgcccacc cacattaagg gtgggtcttc ccctcccagt ccactgactc |
| 261781 | aactgtcagt ctcctctggc aacaccttca cagacacacc aagaaacaat attttaccag |
| 261841 | ctatctaggc atccttcaat ccaatcaagt tgacacctaa tattaaccac cacatagacc |
| 261901 | aatggaataa gttagagaat ctagaaataa agctgcacat gtacaagaac ctgatttttg |
| 261961 | acaaagttaa caaaaacaag caatggggaa aggactccct gggtcataca cagcattgga |
| 262021 | ataactggat agccctatcc agaagatcaa agctggactc tttcctttca ccagatacaa |
| 262081 | aaatcaactc aaggtgggtt aaagacttaa atgtaaaatc gaaaactata aatttttagt |
| 262141 | ttttaaaact aatctataaa aactcttgaa gaaatcctag gaaatactat tctggatatc |
| 262201 | ggccctggca aatccttcat taagaagact ccaaaagcaa ttgcaataaa aaccaaaatt |
| 262261 | gataggtggg acctaattaa actaaagaac ttctgcacag caaaagaatt tatcaacaga |
| 262321 | ctaaacagac aaccaacaga atgagaaaaa tatttgcaaa ctatgcatcc aagaaaagtc |
| 262381 | taatatccag aatctataag gaacttaaac taaccagcaa aaacaaaaca aaacaaaacc |
| 262441 | ctaaaaaacc attaaaatgg gcaaaagaca tgaacagaca cttctcaaaa gaaggcacac |
| 262501 | acggccatta agcatataaa gaattactca acattactaa tcattagaga aatgcaaagt |
| 262561 | aaagccacaa tgagatacta tctcacacca gtcaaaatga ctattgttaa aaacaaacaa |
| 262621 | acaaacaaac aaacaaaaaa cagatgctgg catgactgtg gagaaaaggg aacacttaga |
| 262681 | tgctgttggt gggaatgtaa attagttcag ccactgtggc aagcaatttg aaaatttctc |
| 262741 | aaagaactca aaacagaact accatttagc caagcaatcc aattactggg taaatatcaa |
| 262801 | aggagtagaa atcattgtac tataaagaca catgtactca tatgttcacc acagcactat |
| 262861 | tcaaaacagc aaaaacatgg catcaaccta gatgtccgtc aacagtgtac tggatgaaga |
| 262921 | aaatgtagta tatatacacc atgcaatgct atgcagccat aaaaaagaat taaattatgt |
| 262981 | ctttgcagca acattgatga agctggaggc cattatccta agtgaattaa tgcaggaaca |
| 263041 | gaaaaccaaa taacctcata ttctcacttg taaatggaag ctaaacattg agtacacatg |
| 263101 | gatgcaaaga agagaacaat agacaccagg gcctatgtga gggtggagag tgggagcaag |
| 263161 | gtgaggattg aaaaactacc tattggttac tacgctcatc atctgagtaa tgaaataatt |
| 263221 | gatacaccaa acccccacaa catgcaattt acccatgtaa gcaacctgca catgcattcc |
| 263281 | ctgaacctaa aataaaaatt agaaagaaaa aaataataat taaaaaaatt atctggggga |
| 263341 | aaaataattt acattagtag caccctcttg tcttcagggg atatgttcta agacccccag |
| 263401 | tggatgccta aaactaagga tagcaaattc tattgcbgtc agttggaaca tgtttctttt |
| 263461 | catgtcttca gtccacatat ttaatgactt ttccacttta actaaatttt aatcacacac |
| 263521 | tgtgaccatc acttttacag tttgaggtac aacagcaaaa ctatcattaa tttctttttt |
| 263581 | cttcttcaca atttcatgga tagaagattt gttcttactg tagatcttag caacctcagt |
| 263641 | gtatgatttt cttttccttc cttattaggt caaaaacttt tgccttttca cttaaaggaa |
| 263701 | gcactttaag tcttatctgt ggcatatctg aattgccagc atcactgctc ttgcactttg |
| 263761 | agagtattac atgaaataag ggttacttga acccaagcat tgccttactg taatagtaag |
| 263821 | tctgataact gagaggacta ttaacaacat gggtatacta ttgtatccac aggatgcatc |
| 263881 | cacactaaac aaagggagaa ttcacatcct gggcagggtg aagtgggaag gtgaggaatt |
| 263941 | tcatcacagt actcgaaaca tcataaaact taaaacatga attgtttatt tctagaattt |
| 264001 | tccatttaat atttttggaa ccatgaattg atcacaagta actaaaacca tggaaaagag |
| 264061 | agaactagtc cacatgttgt ttagtatcgc tattacagta attttctttt tcttgtgtgg |
| 264121 | tcttatcctg tgggcttcta gaaaattgtt tcctacctgc ctctcaatcc ccttcctctt |
| 264181 | ctcaactttc attttagcag aggctacatt atagtgaccc tgttggttta ggatttaaag |
| 264241 | aggcatttat ttgatttgtt aaaaacctcc acatgcaccc atgaaataac caaatgacct |
| 264301 | cagggtataa atggagctgc aaacttgtcc aagtcaagaa attcagtata aaaaccttag |
| 264361 | gcaggccagg cgcggtggct cacgcctgta atcctagcac tttgggaggc cgaggtgggt |
| 264421 | ggatcacgag gcagaatatc gagacaatcc tggctaacat ggtgaaacct cgtctctact |
| 264481 | aaaaatacaa aaaattagcc aggcatggtg gcgggcgcct gtagtcccag ctactcagga |
| 264541 | ggctgaggca ggagaatggc gtgatcccgg gaggcggagc ttgcattgag ccgagatcgc |
| 264601 | gccactgcac tctagcctgg gcgacagaga agactcctcc tcaaacaaaa acaaaacaaa |
| 264661 | aacaaaaaca aaacaaacaa aaagaaacac cttaggcaac aacatatatg ctagaattgc |
| 264721 | ctttgctaac tcccctggca aagcaaatga tgtattgaga ttgcttctca gataaggtgg |
| 264781 | ttgaaaatga tagcaatctt aaaaaatgag cagttttcat aaatttgtat acattttgct |
| 264841 | ctatagacaa agagtagagc ttatagctat catggccata tctcaataaa aatgtgttgc |
| 264901 | aagcaggttt gccttgggca tctttcctta ttcctttcag tattccactt cttttcacac |
| 264961 | cctttcccac atgttagatg ttcccagtct gctcattcag ttcgggctcc cttccctggg |
| 265021 | aggcbgtctt cagctcaaaa ctttgccagt gataaaactc cattcaaaac ttatctgcca |
| 265081 | agtggccatt tttacttgaa gccctgccct tggtcaatga cagtgcaaaa actggatagt |
| 265141 | ggcatattaa tcaaataagt cattttctcc ctaggttaaa ccatagggaa tatctgcttt |
| 265201 | caaccctttt ggcctccaaa aaaaaaaaaa aaagcatttt gtatggcttt tcatacctca |
| 265261 | tggtgtccta gcttgagcac tggagcaaag tcagaaaatc tgagcccttg acttatctct |
| 265321 | gtggttcact gctggataat gtccagaaaa ttatttcatg cctttgagct tcagcttcct |
| 265381 | tatctgtaaa aaggagagta actaccttga cttcttgctg agattttaaa gagaacaaat |
| 265441 | gtgaaagcac acacaatcac catttgcaaa gggtaaagtg gtagatggat gaaaggaata |
| 265501 | atgatctgta atttttcctt acatgctagc ctatttaaaa tgtatgagcc tccatgataa |
| 265561 | gtgtggctaa tagtggggga atgtagatga tctagagata ccggtcaacc tacaatgtca |
| 265621 | gctttatgtc attgtgcccc tttcttcttt ggggtttata tgactataat attttaaaaa |
| 265681 | ccaaaacctt atgggaccat tttataattt gtgtcatatg aaattttaat tttgctattt |
| 265741 | atagtgaaag ggaaaaaagg aagtggagta tataggatat agatctctaa ccactaaaat |
| 265801 | aaacctgtga gtaaatgcat tttaagctct tcatgcaaat acatatatat atatatatac |
| 265861 | acacatatat atacacacac acacatataa ttactgatta aaaatatata taaaatcaca |
| 265921 | tatatataca tataaaacca cttggcattc aggttgtata aaaatgttga tttttattca |
| 265981 | agaattgtag agtagatgga aatttaatgt actcaatgta atctctattt gggatgagat |
| 266041 | tctaggggga attaaaattt acataggagt ttctttgcct ggtttagtcc atccagaggc |
| 266101 | ttcagatggt ctgggaacca actaaagttg tgtggagcaa tgtaaaagtc agtatatatg |
| 266161 | tttcttttcc cctggggagg gattcaatag atttcatcgt attcttaaag aaattcttgt |
| 266221 | cctaccaaag gttaagaact aattatctca gttaaacaaa acacataaaa atatgttttt |
| 266281 | atatacatgt tgaattattc aaatcttcag tgcttagtag tttattaatc ttgaaggcct |
| 266341 | agaaagtgaa taatttcaac aactgaacaa aaataaaatt taaaaagaaa ctattttctg |
| 266401 | gtcattagaa caaacttgtt ataaaatata tgtctttttg ctattgcttc tccacttcaa |
| 266461 | cactcatgat catgttttgt tgctatttcc cctatctcat tttttctggc tgctgggtta |
| 266521 | aacttcctgt ttttgcagaa tgaccaaaaa cattggtgat gatggaggtg gagatgacaa |
| 266581 | cactttcaat ttcagctgga aggtctttac cagcbgggac tacctgatcg gcaatcctga |
| 266641 | aacagcagac aacaaattta attctatcac aatgaacttt aaggtagagg caccaacttc |
| 266701 | aaaaacctgc tgtttgtatt tctaagagta aaattggagg cattccatct ggtccagtag |
| 266761 | tgcagtatct tgacaattta catgaaagat taactcatgg tttctgccta ccaagatctt |
| 266821 | gccatggttc tgattataac tggaaaatct tccatgaccc acaacagcac tattattcct |
| 266881 | taaacacttc tcttctctaa atctggtaat aaaataatgt tactggattg aaataggacc |
| 266941 | atgagtaatc agaataagat attttaaaaa ctagaacaat gttcactaat tatttacagg |
| 267001 | aataagcata atattgaagt gaccctaaaa cattattttg tagttttttt tttaattcac |
| 267061 | tggaatgaag atttttagag tgaagactat cctaatatca gagcaatcac agtttgtttg |
| 267121 | caagaattct tccttctgtc ttacttgcca tcgtttgttg gcaatattca cctggtttgt |
| 267181 | ggaatctcag atttagttta cattgtcatt cctcacattc tgatgattgt aaaaataact |
| 267241 | tttgaggttt tgatactttt aaaatgtagc taagatattc tattttgggt ttttgtttgt |
| 267301 | ttgtttgttt tcatggatac aggaagctat cacagaagaa aaagcagccc aagtagaaga |
| 267361 | aaacgtccac ttgatcagat tcctgaggtt tctggctaac ttcttcgtgt ttctaacact |
| 267421 | tggagggagt ggatacctca tcttttgggc tgtgaagcga tcccaggaat ttgcacagca |
| 267481 | agatcctgac acccttgggt ggtgggaaaa aaatgaagtt cgtctctgca tgctttttat |
| 267541 | gtgcttagaa cctgacattt gtttcttttg tgggttatgt ttctttgatg gaacatttaa |
| 267601 | aaaggccacc ctttacctat cccctatcct gcccttacag ttaaccaacc ctgttgctca |
| 267661 | ttttccattc tattctagtc ttatttctaa atctggactg tctgtttttc attttgtgac |
| 267721 | atgatggaaa gttagagtca gaaaagatat taaaaaacac atagtctaat cattatgttt |
| 267781 | tacagatggg aatgataaat ctcaagagat tgcattatat gtttatggtc acagagttag |
| 267841 | gagaagagtc agggtgagaa cctaggaatc ttgacttcga gtccaaggct cttgctgctt |
| 267901 | tctctgcaaa tccttcatct gtcattaaaa gatcaaattg ttgcctgaac tttttgaagc |
| 267961 | agccatgata aagccactga ttcctatgtt gatctcactg gaaacactca gattgcttct |
| 268021 | ccacctttgg gcatgtctgg gcttccctct gttttcatta gaagcctaac tgcagcatct |
| 268081 | ccatctccat ttcctcctgt cctgtacctt actttctctc cctctaaatg ctattcaaat |
| 268141 | gaagaaggcc agctcttttc caggaattaa acattatgac cgtaatttgc ctttagagta |
| 268201 | tctgcccatt tagtgaatca gagaaaggag gttagacata gatcatttag aatcagaaaa |
| 268261 | tcattggtca tgagttgttg ctagagtctg ttcccgttca taaaatgctt atgtgtatga |
| 268321 | tgtgacgtca ttttgagctt gggcattttc aaagtatgtg cagatttagg ctgtgggaag |
| 268381 | tgtagacttc ccacactgaa acttttgcag aacagtatag atcaaaagaa ttaaaatatg |
| 268441 | tctttataag catagaattt tctaaaaaat cttggacagc aaatgatata atctacttac |
| 268501 | agaaaaaggt tccaaataac attctgagta tttcagaagt ggtaccaata gagacgggac |
| 268561 | tagatatttt aaagacttta atatacagtc atgtcataaa aacttgtaaa atataagtaa |
| 268621 | tttagcagag ggcttttatt tatttaaaat gaaaaatgta ggaacacctc aaagcattta |
| 268681 | ctaagtttgc tttcaaaatg gtctcataat tgtaatagta catagaggta acattatcat |
| 268741 | gcagctttgg tccattattt atattggtta tgtttttgta taaataaacc atattaaagg |
| 268801 | atgggtagtt gattatcaat aaaataacta ttctttgtaa gaacctcttt aacatatatt |
| 268861 | ttagggaccc tctgagtcct aggggccaat gaaggtaata atggagattc atgagtaact |
| 268921 | ttacaaattt gataaaacca aatttaaatt tgccttgcat aagtttgcac aggataagcc |
| 268981 | aaattttctt tacattagtc taaaattata gcatgctgat tttgtattat gtcatgctga |
| 269041 | gatttggatc ctgttggtca tacttatata atgttttaat aaaaatgaca aaaatctaat |
| 269101 | agcaattgtt ttctaaatat agtctggtag ttattataat tattcacacc acgaagtcca |
| 269161 | caggagaaaa taggaaatgt ttagtggtga cagtttgagc cgggtatttg ggaaaggaga |
| 269221 | tagtcaccag agttacctaa aatcggtatt tccctattag aaaattagta catgttttgg |
| 269281 | attcttttta ttgacttcct ttagccaaat aatatcatca taaggttgcc aaacatccag |
| 269341 | gaaataaaaa gcacatgttt cctacttttc atgtttctat tccttcttct ttctctctcc |
| 269401 | cccctctccc cacccctgca ttattctctt gtctctgaaa ttatagccca ttctttcact |
| 269461 | tttgaaactt ttctacgttg gcttccataa cattatgtca tgtgttctcc atttgtttct |
| 269521 | ctgagttcct tctgtttcct ttaataaaac tcattgtatg tgtttatgtt ttcggcctta |
| 269581 | aaagtttaca tttgccaaaa ttcaatattt aggcctaggc ccatctcagg gaattcatcc |
| 269641 | attctaactg attaaactag cccatatgct gatgattgtc agattagaat tttcagtttg |
| 269701 | aacctcctcc tcaagcagat tattttccat ttcttttgga aatcttgttg tgaagtgcat |
| 269761 | caccatatca gaatcagtta ccacatagaa caatatgtgt tctctgtgta gagactgatg |
| 269821 | taaattgcca ccttcactat accaggaaac agactgttac tactggcaca tacacacact |
| 269881 | cagtcacatg cctctcttga gaatgtgaat tgaatatttt ccctgtcatt ttattgagat |
| 269941 | caaattctaa acgtgcataa aatagtcatt tttagtctta aaaaagatca aagaagccta |
| 270001 | gctcagaatc ttccaaaatt ctggcaaaaa gcaataataa ctttaaacac cattaaccta |
| 270061 | gtttctccct tgtgcctcct tgtagatgaa catggttatg tccctcctag ggatgttctg |
| 270121 | tccaacattg tttgacttat ttgctgaatt agaagactac catcctctca tcgctttgaa |
| 270181 | atggctactg ggacgcattt ttgctcttct tttaggcaat ttatacgtat ttattcttgc |
| 270241 | attaatggat gagattaaca acaaggtaag ccttgtttct ggattgtcct tgccataaga |
| 270301 | gtgttgttca attcccagtc tcaagtttgc cagaaatgcc tttgaaatct ctgttgtctt |
| 270361 | cattttagtt tctatctctt tgctttctag attgaagagg agaagctagt aaaggccaat |
| 270421 | attacccttt gggaagccaa tatgatcaag gcctacaatg catcattctc tgaaaatagc |
| 270481 | actggaccac ccttttttgt tcaccctgca gatgtacctc gaggaccttg ctgggaaaca |
| 270541 | atggtgggac aggtaatgcc accaacagaa gtgtatggca attagtagat taaaaaaaga |
| 270601 | gagtcaatat ctcttccata agattggctt ttaaaaaatt gcttattagt aaaaatagct |
| 270661 | aagatttgtt gaatgttgac tgtgtgctgg ctctgaattt catgtatatt atctcacact |
| 270721 | gtttgcaaat ctatcccttg aggtaaatac tatttgtatg tcccctttac agttgcatat |
| 270781 | aaaggaagga tagttgaaaa tggtgcaaag agatgtttac tatctgtatt ataataaagc |
| 270841 | actcgattta gatacaggcc aaagcaaata tttaaatgga actctattta gtgttacttg |
| 270901 | tttgctgtaa aacaaaacaa aacaaaacaa acaaacaaac aaaaacagag tcttgggggt |
| 270961 | ggccaagaca gccaactaga ggcagctagt gtgcatggct ctcatggaga gaaatagagg |
| 271021 | ggttgagtaa atatagcacc ttcaaccgaa acatccaggt acatgcattg gatctaatca |
| 271081 | aggaaacaac ttgacccatg gagagcagag aaaagcaagg cagggtgaca gcccacctgg |
| 271141 | tagtgacatg aagccaaggg atccttctat gcccagggaa gtggtgagtg aatgcacgac |
| 271201 | cttgggaacc catacttctc ccgtggatct ttggaaccct tgtgtcagga gatatcctca |
| 271261 | tgaacccact ccacctgggc cttcagtccg acacacagag ctatgtggag tctcagcaga |
| 271321 | gcagccactt aagcacatgc agagacccgg gagccttaga tacctgggct tttctttttg |
| 271381 | ggcttcctgt caaaagtagc tatacctctg gcaaagcagg aggttagacc cccaaccaca |
| 271441 | ccccttagaa aaggggctga atccagtgac agtctgcagg ccccacttca cagcacctca |
| 271501 | cagaatgaaa cccactggct tggaactcca gccagttacc ggtagaaatg ttgcacctcc |
| 271561 | ctgagacaga gctcctgggg gaaagtgtgg gccatcatct ttgctgtttg gttgacttgg |
| 271621 | ccattccatc attcaggctt cagagagtcc aagccaacca gcggctgaag cagaccctga |
| 271681 | gcacagcata gcbgttctat gaaaatgtgg ctagactact ttttcaaatg ggtctcctat |
| 271741 | ccctttcctc ctcactgggt aggacctcct aactggggtc tccaaccacc tcctacaggt |
| 271801 | gcgtttgggc cagcaacggg tctgtacatt tctgggatgg agcttccaga ggtgggggca |
| 271861 | ggctgccatc tttgctgttt cacagccttc actgttgcta cctccagata ataaaacatc |
| 271921 | tgaggtgacc agggacggat gtggggtccc aatatactgc agcagcccta tgaaaaagtg |
| 271981 | accagacttt tacatgggtg cccatcccca tatcggctca ccagacacat cctccaggcc |
| 272041 | taggcctcca gctaccctct gctagaatta tcgatcaagt agcagctctg taactccctg |
| 272101 | gacaggatcc ccaggggcaa ctgaaagttc ctctgccact acctctgcag tagaactgcc |
| 272161 | cttgctaccc ttggactaac aaaggaacaa agaccctaag cgcctcatcc atacctccaa |
| 272221 | taagctgcag ttgactccag gagagaaggc cagtctgtct ctcacaggtc ctacctaccc |
| 272281 | ccacccccct ccttgtcacc agacagggaa cccccagctc ggacccacaa catagaacat |
| 272341 | ccatcctggg ctaattgcaa ggagtaattg ctgacctgca tctcaatggg gtggagcccc |
| 272401 | caggagataa gcaaaagacc cttggccata accatgccaa ggtatcttcc tctgctgctg |
| 272461 | ccaagttgga gaagaaacat aacgctgaga ttgccccaga gctgaagggg cagcccagga |
| 272521 | gtgccaagcc gtaatctaca gccagctctc aagggggaaa gaaatctaca ctttcagagc |
| 272581 | attgagaggg aacatggctg caactgtgcg gcaacacagg ggagcctcac aaccaagcaa |
| 272641 | gagtctacca actgaccaat atgccgaagt gccacctact ggatcacatt ccccaaagca |
| 272701 | tcaacaccaa aaatatctca ctaacatacc cccctctgaa accaaagaca agtactcagc |
| 272761 | ttcaaataaa gaccctgtgc aaagccttgg cccaatgaaa acttccagga aagatgccta |
| 272821 | ttgactgtat tcaatctaca ttgcagttaa aggaacacct acacactgag atgagaaaga |
| 272881 | accaatgcaa gaactctggt aactcaaatg accagagtat tgtatgtcct ccaaacaacc |
| 272941 | acaccagttc tccaacaaga gttcttaacg aggctgagct agctgaaatg actgaaatag |
| 273001 | aattcagact gtggagagga atgaagatca ttgagattca gaagaatggc aaaacccaat |
| 273061 | ccaaggaaaa taagaatcac aataacacga cactggagct gaaggacaaa gtagctgtta |
| 273121 | taaaaaagaa gctaataggt ctcataaggc tgaataacac aatacaggaa tttcacaatg |
| 273181 | caatcacaag cattaacagt agaataaacc aagctgagga aagaatctta gaacttgaag |
| 273241 | actggctctc tgaaataaga cagtcagata aaaataaagg aaaaagaata aaacgggaag |
| 273301 | aacaaaacct ccaagaagta tgggattatg taaatgccaa agctatgaat cactggcatc |
| 273361 | cctgaaaggg atggggaaaa agcaaacaac ttggaaaaca tattttagga tattatccat |
| 273421 | gaaaacttcc ccaaccttac tagagaggca aatttgctgc cagtctttgc taacagtcaa |
| 273481 | atttaggaaa tacagagaat tcctgcaaga ttctacacag gaagatcatc cccaaaacac |
| 273541 | acaattgtca ggttttctca ggtcaaaatg aaaaaatgtt aaagacagcc agagagaaag |
| 273601 | ggcaggtcac ctacagagag aacaccatta ggctaacagc agatgtctca gctgaaaccc |
| 273661 | tacaagccag aagcagtcag ggtcctatat tcaacattct taaagaaaaa aaatcttcaa |
| 273721 | ccaagaattt catatcaggc caaactaagc ttcctaagca aaggagaaat aagataattt |
| 273781 | ttgggcaagc aaatgttgag ggagtttgtt accaccagac ctgctttaca agacatcttg |
| 273841 | aaaggagcac taaatggagt aaggaaagac tatttgcagc taatacaaaa gcatacttaa |
| 273901 | atacacagac cagtgacact gtaaagcaac catacaaaca agccagcata ataatcagtg |
| 273961 | aacaacacaa tgacaggatt aaatccatac atattaatac taatcttgaa tgtaaatggg |
| 274021 | ctaaatgctc catttaaaag gcacagagta gcaagctgta ttaaaaaaaa agcaagaccc |
| 274081 | aatggtatgc tgtcttcaag agacccatct catgcttaat aacacccaca cgctaaaaat |
| 274141 | aaagggatgg aagaaaatct accaaccaag agaaaacaga aaaaaacatg ggttattgaa |
| 274201 | attaaaattc ccaatttcaa aaaaaaaatg caaaaaacaa aacacacaca tgcacacaca |
| 274261 | aatacacttc aaaccaacaa agataaaaaa aaggcaaaga agggcattac ataatggtaa |
| 274321 | agggttcaat gcaacaagaa gacctaacta tcctaaatat atatgcactc aatacaagag |
| 274381 | tacccagatt cataaagcaa gttcttagag acctacaaag agacataggc taccaccacc |
| 274441 | caataatagc ccaggccctg atgaattcac agctgaattc taccagatgt acaaagaaga |
| 274501 | actggtaaca ttcctactga agctatttca cgaaattgag gaggaggagg aacttcttcc |
| 274561 | cagctcatcc tatgaggcca gtatcattct gctaccaaaa cctggcagag acacaacaaa |
| 274621 | aaaagaaatc ttcaggccaa tatcatgagg cccatcaatt caaaaatctt caacaaaata |
| 274681 | cttgcaaacc gaattcagca gcacatcaaa tggctaatcc accacaatca agtaggcgtc |
| 274741 | atccttggga tggaaggtta gttcaacata tgcaaaacaa taaatgtgat tcatcatgta |
| 274801 | aaaagaaata aagagaaaaa ccacatgatt atctccatag atgcagaaaa agctttcaat |
| 274861 | aaaattcaac acccgttcat gttaaaaact ctcaataaac taggtattga aggaacatac |
| 274921 | ctcaaaataa taagagcctt ctatgacaaa cccacagcca aaatcataat gaacggacaa |
| 274981 | aagctgaaag cattcccctt gaaaaccagc acgagacaag gatgccctct ctcaccactc |
| 275041 | ctattcaaca tagtatttga agttctggcc agggcaatca ggcaagagaa agaaatgaag |
| 275101 | ggtatcaaaa taggaaaaga gggagtcaaa ttatctttgt ttgcagatga catgatccta |
| 275161 | tatctagaaa accccattac cccggcccaa aagcttctta agcggataag caacttcagc |
| 275221 | agtctcagga tatatcaatg tgcaaaattg ctcgcattct tatacaccaa caacaggcaa |
| 275281 | gcagagagcc aaatcatgaa tgaactcaca tctacaattg ctacaaagat aataaaatac |
| 275341 | ttaagaatgc agctaacaag ggaagtgaag gacctcttca tggagaacta caaactactg |
| 275401 | ctcaaggaaa tcagagagga cacacacaaa tgggaaaaca ttcaatgctc atacatggga |
| 275461 | agaatcaatt ttgttaaaat ggccatactg cccaaagtaa tttatagatt caatgctatt |
| 275521 | cccattaaac cacaactgaa attcttcaca gaattagaaa aaaactattt taaaattcct |
| 275581 | atggaaccaa aaaagagctc gtatagccaa gacaatccaa agaaaaaaga ataaagctgg |
| 275641 | aggaatcaca ctacctgact tcaaactata ctacagggcc acagtactca aaacagcatg |
| 275701 | gtactggtgc aagaacagac acatagacca atggaacaga atagataact tagaaataaa |
| 275761 | accacacaac tacaaccatc tgatctttga caaacctgac aaaaacaagc aatggggaaa |
| 275821 | atatttccta tttaataaat gttactggga gaactggtag ccatatgcag aaaattgaaa |
| 275881 | ctggacctct tctttacacc atgtataaaa attaactcaa gatggattaa aggcttaaat |
| 275941 | gtaaaaccca aaactatgaa aactctagga gaaaatctag gcaatatcat tcaggacata |
| 276001 | ggcacaagca aagatttcat gacaaaaatg ccagaagcga ttgcagtgaa agcaaaaatt |
| 276061 | aacaaatggg atctaactaa actaaagagc tccagcacag caaaagaaac tatcatcaga |
| 276121 | gtgaacagac aacctacaga atgggagaac atttttgcaa tctatccatc tgacaaaggt |
| 276181 | ctaatatgca gagtctacaa ggaaattaaa caaatttaca agaaaaaaac tggatcccat |
| 276241 | taaaaagtgg gcaaaggaca tgaacagaca cttctcaaaa gaagacatac atgcggcaaa |
| 276301 | caagcatgtg aaataaaact tcatatcact gatcattaga gaaatgcaaa tcaaaaccac |
| 276361 | aatgggatac cagctctcac cagtcagagt gattagtagt aaaaagtgaa aaaataatat |
| 276421 | gctggcaagg tcgcagagaa aaaggaacat ttatacactg ttagtgggaa tgtaaaatag |
| 276481 | ttcaaccatt gtggaaagca gtatggcgat tcctcaaaga gctaaaagca gaactatcat |
| 276541 | tcaacccagt catcccatta ctgggtatat acccagaggt atataaatca taaagacaca |
| 276601 | ggcatgtgaa tgttcattgc agcactattc acaatagcaa atacacggaa tcaacctaaa |
| 276661 | tgtccatcaa tgacagattg gatagggaaa atgtgatata ccatggaata ttatgcagcc |
| 276721 | ataaaaaaga atgagatcat gtctgttgca gaaaaatgga tctattatcc ttagcaaact |
| 276781 | catgtaggaa caccaaatac tgcatgttct tacttatatg ggagctaaat tatgataact |
| 276841 | catgaacgca aagaaggaaa caacagacac tggggtctac ttgagggtgg agggtgagag |
| 276901 | gagggagaag agcagaaaag ataactattg ggtactgggt ttaatacctg ggtgataaaa |
| 276961 | taatctgtac aacaaactcc catgacacaa atttacctat gtaacaaagc ttcacatgta |
| 277021 | ctactaaacc taaaataaaa gttttttttt aatggataat ttggggagca cacactaaga |
| 277081 | aattttaata atgtcaattt ttgttaaata attttccctt aaaagaaaaa aaatactatt |
| 277141 | ttaagttatt tgttttcatg atgattttgt aaataacccc agcccaagac tagccatacc |
| 277201 | ttaccagaga aaagaaataa tgtttcatat ggctttctat cccttaaggg aaaccaagac |
| 277261 | aaataaaacc aaacaaaacc caagaaaaat ctacttcaga tctcgtattt ttatacaaat |
| 277321 | ccaatttata ttttgaaaag cttttatgat ttcatttagt tagaattgat attcctaacg |
| 277381 | tgcagtccat aaattgattt tggggcttca gattatcatt gagaacagct tttgtcataa |
| 277441 | agcttctgta tgttagacaa cagttgagtt tagcactgaa tgtagtgttt gtgaaattat |
| 277501 | ttgaacaatt ttttactgca ttatttggta agataacata aaaattgaag gactagcatt |
| 277561 | agtcttataa aattcatata atttaaacaa atatgtaaaa atatatctta ggcttcaatc |
| 277621 | agatgctcat caaacttaag ccaatcaaga tttcaactgt gaaagaaaaa aataagataa |
| 277681 | atgtgtaaaa tgctagacat gtatcattaa tgattgagta caaaaattat ggtataattg |
| 277741 | aaaattctat tttattttat ttgttaagct ttcgggaacc agataaatcc cccctttgat |
| 277801 | gatatatgct attaatttga ttttcagaca tgcacatttc atacaactta caggaacata |
| 277861 | ttacatatga aataataaaa tcaaagtcat ctgcacttaa tacaggagtg cttgtgattc |
| 277921 | aggatgtggt agacagaata atggccctct ataacagtac acattctaat ctctggaacg |
| 277981 | ataaatatgt tacatggcaa aatggacttt gcagatgaaa ttaagttaaa gatctcagct |
| 278041 | tgaaagaatt attgtggatt atgtgagcag gcttaatgaa atcataagga tctttataag |
| 278101 | caggagataa agggaaaaag tcagaaaatt gagatgtaac agcagaagca gaggttggaa |
| 278161 | tgatgtgctt taaagatgga ggaaggagat taagagccaa ggaatgcagg tggcctcaga |
| 278221 | aactgaaaaa gaaaggaaac atattctctt cccaagtctg cactttgaat ttagacttct |
| 278281 | gacttccaga actgtaagaa gataaatttg cattgtttta aaccactaca tttatggcaa |
| 278341 | tttgttacag caacaatagg aaatgagtac atcatggtac ttgggtctcc ctgttccatg |
| 278401 | gctttttgaa gtgaagtctg gttgtggtgc ttaacacgag tctgttttat cacaactaag |
| 278461 | ccgctcccta aatcttcagc atacgattca agaaaggaaa atataggaag tacagtttta |
| 278521 | gaaactgaag gaatttgaga cgttctcttc tgcacatcct agaacacagg ttggcctgaa |
| 278581 | agctaagaaa cctgcacttt aacaagcaaa aacatctact gaatgggatt aatagaatta |
| 278641 | gctgtaaaga gtaggatctg ggctggccac ttctggagtg aaggaatgga ttaattagat |
| 278701 | aggaatgcga atgcaatcta ggagaatatt catgtttcag atgcagatgg ttctgcgata |
| 278761 | gtcccacaca tactgactta aaagagaaga aataccattt agttaatcct ccaccttgga |
| 278821 | agcccgtgga ttatgctgct tgtctttttt tttttttaaa cctgtctgta gtgattttcc |
| 278881 | ttgcttcatc atattttctt actgccattt tagttggatt tcccccttat ctttcacctt |
| 278941 | cctttctcca aggaagcatt aagctctagt gggaaagtct gcagaagcgg gccaagggaa |
| 279001 | aaggggttgt ggttctgact ggaatgttga ttaggcagat ggaatctctg ggaacaaaaa |
| 279061 | gaacatatag gacataacaa atctgagtgc tggcaaggtc tggcctttaa gagatcaata |
| 279121 | tctgtatcca tgcatctaaa atttctattc tttaattgct taaaaaatat gcctccaaaa |
| 279181 | atagtaaagg cagctgctct gtatgtaatg ttagatatgc accctcattc attgttctat |
| 279241 | gagagcatta cagatgtttc ctcttctcct ccatggatac cctctgcttt gattttctcc |
| 279301 | aaggatgcta tgagactcaa agcacttatt gttttgaata taggataatg tgattgctcc |
| 279361 | tttacttctg cagattccca agcaatcatt tagtttcatc cccaatggat ttgataggtg |
| 279421 | ggcagcagag ccattcctct ttgaagctat atcctgaggt cttcactttg gggaagggga |
| 279481 | gaaagcaaca gctgaagata aaatggctaa tgtgccaagt atttcctcct caaattttta |
| 279541 | cgtttcattc tgagatacgt tatattatta tttccgtttt atagatgaga agactgagac |
| 279601 | ttaggtaatt tgctcaagat ctctgagaaa gtcaatgaca ggagccccag ttcagcctac |
| 279661 | atagtctgct tacagagttt gtgtctttgt tcacagtgct aaattgtccc ccagttagca |
| 279721 | tcaacacaca ctccccaaac tcctgcctgc aatgtctata tttggtggcc ttttatgatg |
| 279781 | ttccttgcct attgttttcc cttcacctat taataaatta tttctttagc tttattgatc |
| 279841 | aaagctgccc aggcatttta actaacatca cgtgtgcaaa gtttttaggt gtggagtgtg |
| 279901 | gggtttagca ggaggcctgt gtgggggctg tgctccccat cctgacaagc acacaaggaa |
| 279961 | ctaggatgat atccttgaga ttggctgggc taccattgtt tggattctct ttcttttaaa |
| 280021 | atatattttt cctatggtat atgaataaac atttaaaatt tttaatacaa tgtttgaatc |
| 280081 | ttcttcagat attttatgtg aaagttaggt cactgcttgg gaaggaatag aatttcaacc |
| 280141 | tttgggatga ggatgcttaa gaggatgtgg aggacttcca aaccactctg aaatgttttc |
| 280201 | ttacctgagg aagtcttcta gacctggcag tagaaaccta aaaatatccc tcatagcctt |
| 280261 | cctctaattg atgctagtta ttagaatctg agctcaacat tgcctggaaa gggtgggcat |
| 280321 | ggaggcatgt gtctgtagtc ccagctactc aggtggctga gacaggagga ttgcttgagt |
| 280381 | ccaggagttc caggtcagcc tgggcaacat agcaagaccc ctgtctctac atacatacat |
| 280441 | acatacatac atatatacat acatacatac atacatacat acatacaaag aactgcctta |
| 280501 | ggaaaggaaa ggagacaaag agtaggaagg agaaaaatta tgtccaaaaa taaccacagg |
| 280561 | aatactatca ttcattcatt ttttatttat gagtgtacag aaattgctgg gcattgtgtg |
| 280621 | attatataat tggaagtaaa acacttctca gcaaatgcaa aataactgaa atcataacaa |
| 280681 | acagtatctc agacaacaga acaattagat tagaaatcaa aattaagaaa ctcactcaaa |
| 280741 | accacacaac tacatggaaa ttgaacaatt tgctcctgaa cgactcctga gtaaataatg |
| 280801 | gaatgaaggc agaaatcaag atgttccttg aaactaatga gaataaagag accatgtacc |
| 280861 | agaatctctg ggacatactt gaaggaactc ttcaaggaga actacaaacc actgctcagt |
| 280921 | gaaatcagag aggacacaaa catatgtgaa aacattccat gctcatggat aggaaaaatc |
| 280981 | aatattgtga aaacggccat actgcccaaa ataatttata gattcaatgc tatttccatt |
| 281041 | aaaccaccat tgacattctt cacagaatta gaaaaaaaac atttttaaat tcatatggaa |
| 281101 | ccaaaaaaga gtccttataa ctgagaaaat ccaaagcaaa tagaacaaaa caggaggtgt |
| 281161 | cacactacct gacttcaaac catactacaa ggctatagta atcaaaacag catagtactg |
| 281221 | gtgcaaaaac aggcagacag accagtggaa cagaataggg aacacagaaa taagatcaca |
| 281281 | gacctacagt catctgatct ttgacaaact tgacaaaaac aagcaatggg gaaaggattc |
| 281341 | gctatttaat aaatgatgct aagaaaactg gctagcctat gcagaaaatt gaaatgggac |
| 281401 | cccgtctata caccttatac aaaaattaac ttaggatgga ttaaatgctt atatataaaa |
| 281461 | gccaaaacta taaaaaccat agaagaaaat ctaggcaata ccatttagga cataggcatg |
| 281521 | ggcaaagatt tcaagacgaa aacatcaaaa gcaattgcaa caaaaacaaa aattgacaaa |
| 281581 | tgggaactaa ttaaactaaa gagcttctgt acagcaaaag aaactctcat cagagtgaac |
| 281641 | agacaaccta cagaatggga gaaaattttt gcaatctatc catttgacaa agatctaata |
| 281701 | tccagaatct acaaagaact taaacaaatt tacgagaaaa aaacatacaa ccccattaaa |
| 281761 | aagtgggtga aggacatgaa cagacacttc tcaaagaaga gattcacgtg gccaacaacc |
| 281821 | atatgaaaaa acactcaaca tcactgatga ttagagaaat gcaaatcaaa accacaatga |
| 281881 | gataccatct cacaccagtc agaatggtga ttattaaaaa gtcaggaaac aacagatgct |
| 281941 | ggtgaggctg cagagagata ggaaggcttt tacacagtta gtgggaatgt aaattaattc |
| 282001 | aaacattgtg gaagacagtg tggcaattcc ttaaagacct agaaccagaa ataccatttg |
| 282061 | acccagcaat cccattactg ggtacatacc caaaggaaaa taaatcattc tgttacaaag |
| 282121 | atacatgccc acgtatattc attgcagcac tattcacaat agcagagaca tggaatcaat |
| 282181 | ccaaatgcct atcaatgata gactggataa agaaaatgtg ctatatgtac accatagaat |
| 282241 | actacgcagc aataaaaagg aatgagatca tgtcctttgc agagacatgg atggagctga |
| 282301 | aagccattat cctcagcaca ctaacacagg aacagaaaac caaacaccat atgttctcac |
| 282361 | tcataagtga gagctgaaca gtgagaaaac atggacacag ggaggggaac aacacacact |
| 282421 | ggagcctggt cgggggtaag ggagaaatga gaacatcagg aaaaatagct aatgattgca |
| 282481 | ggacttaata attaggtgat ggattgatag gtgcagcaaa ccaccatggc atgtgtttac |
| 282541 | ctatgtaata aaactgcacg tgtaccccag aacttaaaat aaaaaaagaa gacataaaaa |
| 282601 | atttaaaaaa atacacaatt caatggtttc taatatattc acatagttat gcatccagca |
| 282661 | tcacgatagc attttgtttt aggggcttac aatataccat tgtgcagaca tttaatttgt |
| 282721 | tttatttaat tagtctccca agcatattca aatttttccg tttttaatct ggaagaagca |
| 282781 | gtactttcat gattgtatgt atagatttgt gcacatgtgc aaatattgct gtcataggca |
| 282841 | tttctaaaag tggattttct tgatcaagct ccaaacactt taaaatgtga tggatcttaa |
| 282901 | caaatttctt cccaacgttg tgcccggttt aacttcccca tcagtttatg ctcatgctag |
| 282961 | ttcaccacac tcaccaatat gaagaagcat ggattttaca tttgtcattc gtgaggcaac |
| 283021 | aactcattgt tttcatttgt atttttatta tgtggggttt cggtattttt caattttaac |
| 283081 | ggaccgtttg tatatgttct tctgtgaatt tttaatttgt gacttttgct ggtttctttc |
| 283141 | tttattggat tgccaatcta tattcatact gatttgtaaa atcttttaat gtgttagcaa |
| 283201 | tactcaccct ctatcatata cgttgcaagc actttttcca catttgtctt tcaagtctgt |
| 283261 | aaatagtgct attcaccaca taaaatttta aactgctgtg tggccaatat tttcctgttt |
| 283321 | ttggctctcg tatcatgttt atgaaatttc tcagtaagaa gaaaactttt gatttagtaa |
| 283381 | acgggaaata attatttttt aagtaataaa gtcagggtga aatatttttt atttacttga |
| 283441 | tatgactagt tgttttcttc ttttgccatt aattgcagtc ttcaagccaa tacttcttta |
| 283501 | gaaatatgac agatttaata ccaacactag gaccatttga agacagaact gtgtgtttta |
| 283561 | atagagataa tatctcaaca ggagtttgtg aggctgacag tctctgatgt tctgaccacc |
| 283621 | tacgtcacaa tcctcattgg ggactttcta agggcatgtt ttgtgaggtt ttgcaattat |
| 283681 | tgcbggtgct gggacttgga gtatggatat gtaagtatga tgttaatttt gctttttttt |
| 283741 | ttttttaaat ttatttattt tttattgata attcttgggt gtttctcaca gagggggatt |
| 283801 | tggcagggtc ataggacaat agtggaggga aggtcagcag ataaacaagt gaacaaaggt |
| 283861 | ctctggtttt cctaggcaga ggaccctgcg gccttccgca gtgtttgtgt ccctgggtac |
| 283921 | ttgagattag ggagtggtga tgactcttaa ggagcatgct gccttcaagc atctgtttaa |
| 283981 | caaagcacat cttgcaccac ccttaatcca tttaaccctg agtggacaca gcacatgttt |
| 284041 | cagagagcac agggttgggg gcaaggtcac agatcaacag gatcccacgg cagaagaagt |
| 284101 | tttcttagta cagaacaaaa tgaaaagtct cccatgtcta cttctttcta cacagacacg |
| 284161 | gcaaccatcc gatttctcaa tcttttcccc acctttcccg cctttctatt ccacaaagcc |
| 284221 | gccattgtca tccgggccca ttctcaatga gctgttgggc acacctccca gacggggtgg |
| 284281 | tggccgggca gaggggctcc tcacttccca gtaggggcgg ccgggcagag gcgcccctca |
| 284341 | cctcccgggc ggggcggctg gccgggcggg gggctgaccc cccacctccc ctcccggacg |
| 284401 | gggcggctgg ccgggcaggg ggctgaccac cccacctccc tcccggatgg ggcggctggc |
| 284461 | cgggcagagg ggctcctcac ttcccagtag gggcagccgg gcagaggcgc ccctcacctc |
| 284521 | ctggacgggg cggctggccg ggtggggggc tgaccccccc acctccctcc cggacggggc |
| 284581 | ggctggccgg gcggggggct gacgccccca cctccctccc ggacggggtg gctgccgggc |
| 284641 | ggagacgctc ctcagttccc agactgggtg gctgccgggc ggagaggctc ctcatttctc |
| 284701 | agacggggcg gctgccgggc ggaggggctc ctcacttctc agacggggca gttgccgggc |
| 284761 | agagggtctc ctcacttctc agatggggcg gccgggcaga gatgctcctc acctcccaga |
| 284821 | cggggtcgca gccgggcaga ggtgctcctc acatcccaga tgatgggcgg ccgggcagag |
| 284881 | gcgctcctca cttcctagat gtgatggtgg ccgggaagag gtgctcctca cttcctaggt |
| 284941 | gggatggcgg ccgggcggag acgctcctca ctttccagac tgggcagcca ggcagagggg |
| 285001 | ctcctcacat cccagacgat gggcggccag gcagagacgc tcctcacttc ccagacgggg |
| 285061 | tggcagccga gcagaggctg caatctcggc actttgggag gccaaggcag gcggctggga |
| 285121 | ggtggaggtt gtagcgagcc gagatcacgc cactgcactc cagcctgggc accattgagc |
| 285181 | actgagtgaa cgagactccg tctgcaatcc cggcacctcg ggaggctgag gctggcggat |
| 285241 | cacttgcggt ttggggctgg agactggcct ggccaacaca gcgaaacccc gtctccacca |
| 285301 | aaaccagtca ggcgtgtcgg cgcgagcctg caatcgcagg cactcggcat gctcagtcag |
| 285361 | gagaatcagg cagggaggtt gcagtgagcc gagatggcag cagtacagtc cagcttcggc |
| 285421 | tcagcatgag agggagaccg tggaaagaga gggagaggga gaccatgggg agagggagag |
| 285481 | ggagtaattt tgcgcttata aggtttttcc tttgtaattc gtcatttaca cacactatat |
| 285541 | aagagaaagg catcatagag tttagtggat gggatcaccg ttttaggtgc aataaatgaa |
| 285601 | gaccaatgag tacatttata atgcagagaa gttcagactc gagtatcatg gtttacaata |
| 285661 | ttctagtaaa attagcacaa gatagcaaaa gcaatggacc tcaacttgtc agctgaaatg |
| 285721 | ttgaactcag taaatcttgg ccctgtagcc caccttctcc ctataagttt actagcattt |
| 285781 | ctagaagcag ggaaagatgg ctaatatcaa ctgcatattc tatttatgcc tttgaattct |
| 285841 | actaaactcc tggatactat catatgccag tcactgtgca gtagatgggg tggataaaag |
| 285901 | gatttgataa atatggtact tgcttcacaa agttcacagt ctaataagag ctcgaaagag |
| 285961 | acacacacaa gaggctgcag aaggttcaag gaaggcctag tttgctgtca tgcatctggc |
| 286021 | aagtggtgga gctgaattct ggcaggggcg gcttcttttc ctgaaacaat ggctcaggtc |
| 286081 | tctcagaacc acactggtgc tgactccatc accagctttc cttctatggt catcctcagg |
| 286141 | tcccccaaca aagtctgagg gcaacagcac actcatcacc ctgtgattag cacattgcct |
| 286201 | ggtttccact gtcctccttt gagtcaaact ggtctcagtc atcctggaaa atatgacctg |
| 286261 | gggggttggt agtactcatt tgcttaaaat cttccagtgg cttctcaagt acttagaatg |
| 286321 | atttccaaat ccctttctag gccttcagta ccctggatga tctgctcctt ttctttctct |
| 286381 | ctcaagtcat ttgctacctc ttttgctctt gctttgtaa gttcacatgg gcttcttttt |
| 286441 | atgctcaaac atgggctgct ttttatgctc aaacacaaga tggtccttgc atttgtaatt |
| 286501 | gctgtttctc ccgcaagggg tgctgttgcc ctagagctag gaatggctgg tttcttcttg |
| 286561 | tcattaaaga ctcagctatt atgttacctc cttagtgctc ttctctgacc acctattcta |
| 286621 | atgtagccaa tcaggcactg tttatatcaa cacactgttc tgttttcttc ataagatgtg |
| 286681 | catctatttg acattactct tgtaaaatgt actcatggtc tatctccctc tgttagaaag |
| 286741 | aatactctat gaaaatagag gccctgacct ctcttatttg aactgtgtga gtcacgtagc |
| 286801 | aggcagcatg tgttggctgg ctgtttgaat gttgaacctt ctcatttttg caccatcaag |
| 286861 | gcagcactgt ctattgccag aactctgcct ccttcgaagc atgtgcccat ggagtctcag |
| 286921 | tgtttgtaat gtttttgagt tattgagcca gtaatgcttc tctcctcacc agtgagggaa |
| 286981 | tatcctttat agctgatctt ctatctcttt tatctcctgc cctctagtct ctggatgagt |
| 287041 | ccaacagtta ggactgtttt caagatttca ggctagatct gtctggctgt ctgactttga |
| 287101 | gatggtacct tttataaggt cgtctccatc accctcattg aaagtgcctc ctcggactgg |
| 287161 | ccaaaagcag ttaggtaatg ctcttcggtg aacgacagtg aggtaatggc tacagataca |
| 287221 | ggatgctctt gtgttgcttt cccattcttt gctatgaggt attcagctgg cttggtttca |
| 287281 | tttctatttt ccttacctga ttcagctcct ctgaggcaga cataaaaatt gccatttctt |
| 287341 | ccttagacca ttgcttggct tgtacttacc ttttatccct tggctgcctt cttcttaagt |
| 287401 | ggcagaaaat ctgagtgcaa atatgaagtc aatattacat gacctggatg cataaaagct |
| 287461 | gtaattaaat gactgtaatt tattatttat tgactgctga cagtgttcct gggttagtag |
| 287521 | aaagtacagg aaaaaagaca agttcctgtt ttaggtcaca gagaaggaat tttctcccac |
| 287581 | gttacctttg gaaggctcaa aaaagctctg tcagaatcaa aacaagtttt aaaacattaa |
| 287641 | tgatggatga cgaatctttg atgtagatcc cattgtcatt gacttaccaa atggatagct |
| 287701 | tacacgggtg agggcctact aggtgcaagc cattatgaac aaactctgtg ctaggtgata |
| 287761 | aaggaataca gggatgtaga agatgtagac ttacgtagag gttgcaaatt tggcctgcag |
| 287821 | ggtagattca gctaatggat atttttgttt ggtatgccca gtggtggtgt ttgtttttgt |
| 287881 | ttcatttttt atgagctaat attttaaaat gagagctctt acataaaaac acatggattt |
| 287941 | attgtttttc ttgaaaggct ggcaacatta aatctgtatt cccactaggg agaaattggc |
| 288001 | tggagttgag aaaccactgc catctgcagt tgggatattc attctccagt tcctcatact |
| 288061 | cttcaccact cttttctctt acaagctccc gatataaata gagaaatcaa ttgtatggcc |
| 288121 | cttgaagaga aagtgtgtgt gtgtttgtgt gtgtgtgtga gagagagaga gacaggcgtg |
| 288181 | catcagtaga aatgcagaca cagaggagga gacatttgaa ctaagtcttg aagaatgatc |
| 288241 | aggatttcaa gagatgaaag aaagaattcc cggtggaggg acaagtgtaa gacagagcac |
| 288301 | cagagataga tagtgtgggc aatccaggca tagattaaaa aaaaaaaaaa aaaaaactaa |
| 288361 | aatgaggctg ttttagagca gaaaaggcta ataagaatgt ttctctgcta ttctcctaga |
| 288421 | cctgctaatc aataatatgg tttcagaaag tacctgtcat gaagggaagg acaacctgag |
| 288481 | ataggcagag gacatacttg agaattctgt cagaaataaa agataaaatc atggtccttt |
| 288541 | ctactgtatg catgcatgtg catgtataca cacacacaaa gcacatgatt tttttcttct |
| 288601 | gaattccaaa aataatgcca gtttattgta gaaaatttgg aaaatataac aaagctaata |
| 288661 | tgaaagtaaa aatcataggt aatctctgcc actaatgaga ttaattattt ggattgttga |
| 288721 | atcaaatagt gttagataac aagtgctaca tatttatata tagtaacaga tttttataaa |
| 288781 | tatacacata tatacttttg tatacgtata tatgtgtgta aacaggtagt agagtagact |
| 288841 | ggttacttgc ttttatttgt tttacctaac actgatcatt tcccaatgct attaactgtt |
| 288901 | gttttgtaac gtttttaatt tgctttaagt tgaactgtct aattattctt atgataataa |
| 289021 | gtgttgtgat gacatttaat tatatccatg tacataagta tcttcataga taaatcatag |
| 289081 | tggatgtaca aattattttc ttatgataaa tttctctgag ttgaattgct cagtcacagt |
| 289141 | agagggcttt aaaaaagtct attcactttt attacaacag cctctgtaga gactctgcag |
| 289201 | atttcagcat tagattcaga gatctataga atgctaaagt taggtggatt ttgacacaac |
| 289261 | cagttgtaat gcatgtattt aacatatgaa aaattgcatc tgtgaattga aatgaggttc |
| 289321 | ctaagtttat gtagccagag aataataagg acaatgaagt acacttctgg acgaaaatgt |
| 289381 | tatgttaaat gtattgattt ctgtactggc tctatctgag taagcbgggt taaaagggat |
| 289441 | tgtgaatatt tttcaagaca caaatttatg gtcataatgt tatcacaaca aacctatgaa |
| 289501 | gattaactaa ggtggaaaaa ttactggctt ggagcagatt accagaccaa tgtaagttaa |
| 289561 | tgtaatgtaa gttaatgtct ccaaaggctt ctctgagcac agttctcagt gtttcagaag |
| 289621 | agttcatgag gagtagaata gtttgtccct aggaaggatt ttaactgctc taatttaata |
| 289681 | caaagtgtgc cctcttgtaa actttgggtt ttctttggca gttcaatgca gaatatcttt |
| 289741 | atcttcactt cagttttcat tgacaccatt cttactattt accagcaaag caagtaaaga |
| 289801 | atattcaaga agaaatagaa ttagattcag ggcatgatgg ggtaagggaa tgttcaataa |
| 289861 | actcagcatg gtgttcacag ttgtattttt aaggtgatca ttgctcactc gtaagtgaga |
| 289921 | gccgaacagc gatgggagac ttctttaaag tctgatgaag gatgctcagg attatccata |
| 289981 | aataagaggc atataattga ctgactacct tagtggattc ccaatcatat tatattgtat |
| 290041 | tatgtcacat ctcaatgtac tgcttatttt gtatcagaaa ctctacaaaa tactttacat |
| 290101 | gtattattcc atgtgatcgt accagcaatg ctgtgaggtg gtattattgt tcctacttta |
| 290161 | cagtaaactg aggcttagag gatgttaaat aattgctcaa agacacatta atggttagtg |
| 290221 | gtagagttag gcctgaaact cgagtctcat caccttcaaa gccatcaacc aaatttccat |
| 290281 | catgttgaat ttacaccgta atgcttgact ctagaaattt taaaaattct gccaaaactt |
| 290341 | gaataattca tacactaaac ctatggcttt tggaactcaa agtgctcata ggcttaggca |
| 290401 | ggctacctgt ggtagaatgg agttagcaaa tgaactggga tacagaatta gaagatcaac |
| 290461 | tgttctctag ctgggaacgt ggagatatta tatatcagtt cgggaaggta gaatgataaa |
| 290521 | gaatcaggaa gaaatcatca gcatcccata aggggttgag aacatgggaa cagggacaat |
| 290581 | gcagactgta ttttaagtgt tccttgaata tgtggacagg atctgtctgc agttaacagg |
| 290641 | atcctattct caactagggt tgaagtacaa agcctcaagt gctcaatcat cctagcagac |
| 290701 | gaggctagag tttgtcctag atcagtgctt ctggaatttg agcatacatc agaatcccct |
| 290761 | gaggacctgt gaaaacagca tttctgattc agtagatctg aggccaggcc cgagaatgtg |
| 290821 | cttttctgac aagttcccag gtgatgctgc tgactctcgt ctgaggacca cactcaaaag |
| 290881 | ccactgctct ggagccacag cccagactag cttgcagctc taagcagagt gattccagct |
| 290941 | gtgagaaatg ggatttatgt gcagaaggtc aggcaactca ttccacaaca gaattaattc |
| 291001 | tctctgagat cctgaattgc atttcatatt atccacaggt tggcaatctg cctcaaataa |
| 291061 | tgaaacttgc agagagagaa gagatcttcc caggcttttt ctttgtcaag tgagcaccat |
| 291121 | atgtcttccc tctgggtgga tgactgactt agcacatcac aagcaaaggt ccaaaaatta |
| 291181 | aacaacatca tttccaatcc ctcaactatg atggacttta gcccaaatcc acatgggctt |
| 291241 | ttctgatgct ggcccccaac agtcttacca ggaatgagca gtcattagtg agtttattac |
| 291301 | acttagctca ttcaaataat tttatgatct caaagttcaa gagaatctgt ttttttttct |
| 291361 | ttttccctaa ccgtttttta cctttgagct ttttctattt tagagacaaa caaaatatat |
| 291421 | tgcaaattga tttatattta ttaaagtatt gctttaaaaa tttaaaatat aatataacct |
| 291481 | aatgtttata aaagcctacc tctaaaacaa tgagcactga cttttatact acatatgtgt |
| 291541 | agtttttctg ccttctcttt tgttttagtt ttaaagtgag agcttctgtg tttattgcat |
| 291601 | aagttaaatt ccatttctaa ggtgtttaca gagaagcaat tctttcataa ctggttctaa |
| 291661 | ttttcaacca ataacgtatt tttagactag aaagggatct tagaaatgaa ctagttcaga |
| 291721 | ggttggcaaa cttttgctgt aaagggctag atagtaagta ttttagcttt tgcaagccat |
| 291781 | ggtgtctcta ttataactgc taaatgctgt tgttttagta ccagtcatag ataatatata |
| 291841 | aatgaatgaa tatagctgta ttccaataaa atttatttgt taaaacaggt ggcagtccag |
| 291901 | atttggcaca tgggccatag tatgctgacc tttggtctag ttcatttttg atttatgtgt |
| 291961 | caggaaacta agcctaatga gataagtgac ctgcccagct tacctagcta agaaatggta |
| 292021 | gagtgaagtc taaaatgcag gtttgaatcc cagtcaaatg tctgttctac aaatccactt |
| 292081 | ggtccagtat ggccaccctg tatcctttct gtgcatcagg ccaatttata gatcaagggg |
| 292141 | gttgttcctc cagcbgtccc tgctgcaaac acagcaggaa gctagggacc ctagggtgtc |
| 292201 | ctttatgctt ttctagtcaa ctagttataa aatgtctcat ctctaatgac cctcaggagc |
| 292261 | tctatgtgct tgtgagagag tgaagaaaac aggcatttaa aacgttgagt tttctccacg |
| 292321 | ttcagtagtt tggtgggcca cctcatgata ccattctaag gaggcgaagg cttaagattt |
| 292381 | gatttttaaa cagagacaga gaaaggctgt tccaaggcaa cagatgggta cgggccaacc |
| 292441 | gagtaagcat ttttcagatg tgttctgtga tcacacaggg ctccagggga aaagtgctga |
| 292501 | agatcatcca gagcagggca ctcccccaga aggaaaaaga ctcagagtta atgttctcca |
| 292561 | gataagaagc agttcatttt tctccatgaa ggacacattt tttttccatt gatgatttca |
| 292621 | gcacaaaaac tgtcaaccaa acctttttgc ctcttgtgaa cattgccctt ctagaaagtc |
| 292681 | ggcaaactaa cgaaacactt tgtagctgtc cttcaacagt atctagagtg atactgcagt |
| 292741 | tatccaggtc atctttgttc tgtttaatac acaggactct ttattatttc attatgtatc |
| 292801 | atcctatcat tttaccttga gggttaacca ttcctgttat gccatacttg ctcttctttc |
| 292861 | cctgaatctg ttacaaggga gattgtccct atgtttcagt tgaataggag agtaatactg |
| 292921 | ccttttagga aagtgatgaa gttgaggcct ggattcatga gactcttgag gccaagcatt |
| 292981 | tgaaaattga gaatggtttt gttggagttc caaatcagga accaattcta tctttttaca |
| 293041 | aatatatttt tgagttcttg ggtttgggag gaacagaggc tctcttggat catctttgtg |
| 293101 | tgtgtgtgtg tgtgtgtgtg tgtgtgtgtg tgtgtgtgtg tgtgtgttgg ggagggagct |
| 293161 | gttatgggga tatgtgtggt ctgaagcaca cctggagata ataagatctg agcagtgcta |
| 293221 | ggaacctgcc aatgtcttgg tgtctttttc gtggtcttct tggccctctc atttcgtatt |
| 293281 | tactgcattc ttattccctt attaccagtt ggtcagtctc ctctgattct ctatcccaaa |
| 293341 | ttaaataaga gccagtttta ttgcccttgc taattagcat ttactccatt gttagaatct |
| 293401 | cctgttagga tgcctaatgc atcaccagga agcctttaga tgggccaccc tatcacagtt |
| 293461 | aaccacttcc attctattgg ttttttgtgg ctcctctgta caaaaaacag cctagtgttg |
| 293521 | ctgccctgag catgaaggtt aatatccaag acatggagac tggacattga attgagtaac |
| 293581 | ttcttaagct ttttttatat ttcttttgct gtatttactt tatgttaaag ttaattcaca |
| 293641 | ttttcattga aaaaattttt aatatgcaaa acttgaaaaa gaaaaggaaa actatagttc |
| 293701 | atcacccgca tataaagtat gactatcttg ctgattattt tccagtcttt gtgcatagtt |
| 293761 | tcatatagtt gattaatgtg tggctttata ttttttgttt aacattagag cacagttttc |
| 293821 | ctcatgtaca taaatattct ttcaaaatga ttgtaattca ttgcaatata tcatagtata |
| 293881 | aatatactgt atatacataa ctaatgttca acacttagat ggtttcatca ttgcttgtta |
| 293941 | aagtttatca tcacatcaga taattttatt aagacagatt ctagaagttt tcttacagaa |
| 294001 | tcaaaaagaa taagattttt aaagctttta atatgtactg tcaaattgtt tttctgaaag |
| 294061 | gttgctacag tttttactgt caccagcagt gtataaggga gcctgtcttg ctgtactttc |
| 294121 | atagcattgg atgacagcat ttaaaaatga gtctcactgt gtttaaaaaa aaaaaggtgc |
| 294181 | tttctttgtt ttatttatat ctggttactt atcctagtga ttattctgct attgttgctg |
| 294241 | aagggaagta attgaaaccc tagccatgcc tatgcagaac aatttgcctt tcagttttga |
| 294301 | ccatgtgaga cgctaatcca atgaacattg tgtctcctct agccttcata caccgaattc |
| 294361 | gacatcagtg gcaacgtcct cgctctgatc ttcaaccaag gcatgatctg gtaggccagc |
| 294421 | tgttggacag cttatcactt acagaaaagc ctcccaggtt atttttgcat acagcattga |
| 294481 | atcctggtgt tagaggagaa tacaatcggt gtctaactcc atatttactt ttgcaaagtg |
| 294541 | gatgcctgta tatagtttcc ttttctttca tatcttaaat gtcagtcatc aaaggtgtct |
| 294601 | ctctgttttg caaagcactt cttcatcaaa taatgggagt ctgtgttctg gttagcagat |
| 294661 | ttatttgagg actatatttt tatattttgt gacgccagga tctaatatta aaacattaat |
| 294721 | aattacatta ccacatgata agtatgaaat aaattaacat aaggcctttc cgagagaccg |
| 294781 | agtttatttt gttgtcacta ctaaatatat ttaaaatgta acttatagct acttttaaaa |
| 294841 | ataatacctt atcaaatcca atagtttatc gagatgaaac gcatgtttta ggaaggtcga |
| 294901 | gttaaaatat gatcttcatt ttgtaacttg ttttataggg ccatggcaca tcagaattat |
| 294961 | tttttttgat gtgtgaaggg ttgtcatatc ttttatcaaa ttagagcatt ctcactgtaa |
| 295021 | acttgacagg ttaagatgag tcgatacagt agaaatcgtt tttgagttta aaaactgaat |
| 295081 | cacgacatcc gattcatctg gggaccatgt gtcttggaaa tctgtcactt cagatttcca |
| 295141 | gcctacaagc agttacaata aggctacttt atcctgggga catgtgccta agacaatatt |
| 295201 | atgctgccaa ccataccatt gtcctattca caagtgccgg tatagactca gtttttgaac |
| 295261 | aagtttcata tattgtacca atagatctgt cctgaattat taaacctttg ggtgctgaca |
| 295321 | gacaccggac ccctctgcaa atgctaaaac cgtcttgctt ctgtcacaca aagacaactc |
| 295381 | tccagggctc catcgtcctg ccatgggatg actgttaaat tcctgtgcct gactaggtga |
| 295441 | atcaatatgg aaaagatatt ataaaaatat tgtttcctcc tttgtcaggt aggtttaaaa |
| 295501 | atctaatttt tattcccttc ctttctaaat tttccaagta tgactcacat gtccagactc |
| 295561 | actaaggaag gaaatgcatc ctacattttc caagcaggga ggaaagtcaa ggtggcccga |
| 295621 | ggaacgtcta ctgacacaga agtaaaggga gcagcttctg tgggttctga gctttcattt |
| 295681 | cctggagttc aatttgctgt tatggcagaa aatggctaac acggctatgg gagtaatgag |
| 295741 | aaaccactgt tctatcagtg gcttttacac aaagtgtcat aaatccttca ggcctcatgg |
| 295801 | gtaccattca cgcagacata tgctgcacgt cgattgtgtt ctttcagagt taagacaaca |
| 295861 | gattggtcta gaaatttgtt tttattgtca gaaaagctgt tataatggct gtcctgcttg |
| 295921 | gtttcactac tgattttgca actatgttag tccactattg ttcatattga aattcttaaa |
| 295981 | tatctaacat gtttttccag ttctctcgtg ggcagatcca ttatcaaaat ctctgcactg |
| 296041 | tctttttctg tttattaatt cttcagacag tggtcaagct tgcttctggc taatttgaag |
| 296101 | agaatattga gttttttgag catacaaaga tattcacaca ctttccaaag ttccagaagc |
| 296161 | aaactgacca ttattctatt attcaccttc acgtcaatgc ttggcacaaa tattcttttc |
| 296221 | ctaaccctag gaggctgatg agggaggggg atgtcatgta gagggaggtg tggggcaggt |
| 296281 | gtaatgtaaa attagtaggc tgactctgca caaaggtcct cacacacaat cgttccatta |
| 296341 | ttttatgaag catgaggata taatgcctga gcttaaccat tttctacaga aacaattaaa |
| 296401 | aatgtacggg gatgttttat tcaattccat tggcttgtga ctggagatat tgaattactc |
| 296461 | ctttaggaat cgctgagtag tggaggttat ttacgcagat gttgttaaca tcaacattta |
| 296521 | tagataaaat ccattttgct atgatttggt tgaatgatta gtaaccttta tttacgcact |
| 296581 | tattttttga aatacacatg atagtttctg tgctaatatt tttaaatgat gaaagcacag |
| 296641 | aatatcttta tcatagcagg atattgagaa atggcagaga acacacagtg agagagaaaa |
| 296701 | tgagttttgg acattgctcc aacactgctt cactgggtac tctcccgcca ggtttatcac |
| 296761 | ctttttttct tcactttctc agttgatcag atgtgaataa tttacctgac atgtacatgt |
| 296821 | cgctgatgta ataaggagag ttaaataaaa gaagctttca ttttctttga tcttccagga |
| 296881 | agaaaagaat tttataaggc taaagtgtta caatgatttt tactgacaga tgatgggatc |
| 296941 | atgcctgggt aagggactta gtgagccgag ctgattagta atgcatgagt aaagatagga |
| 297001 | tgagcattaa cattcggata ttattatgcc aggacttatt tttctagctt atggctcttg |
| 297061 | agttatttgt ttaaaatgtc tttgtgatac ttattttgaa attttgaaat ttatttatag |
| 297121 | atgtatacac aaataataag catatacata caaacaatgt gcacgtatat aggtacaaat |
| 297181 | atatgtggtt ttaggcaagt ctcgattttg aattacgtgc aaaattatat tttagaattg |
| 297241 | atcaggttat gcagcaatga aacaacctca aaatcgtagc agctcataaa aggaacatgt |
| 297301 | tcaatgtggg gtggaagaag agctttgctc ttagaactta ccttcacttt agagacccag |
| 297361 | actgatggaa taggatccat ctgaaacatt gctatcacac tagcagagaa tgaagagaag |
| 297421 | gaaaaccaca caggactttt aaagtttcta cccagcagta aggacctaat ttctattcat |
| 297481 | actttattgg ccaaaacaag tgatgtcacc acctgatttt taggaagact caaagtgtat |
| 297541 | tacatcaaca tgtattggta ttggggagta taatctagtg ctttgcaatt ctaacccatt |
| 297601 | agctgggact atcacttgct tacttgctca cttttttggc gttccctctc tcttgctgtc |
| 297661 | ctgtaacaaa agtatactgt attgttttaa aatctgtctt ctcattagaa aaaaatagtt |
| 297721 | taacttccta caaagtcttt gttcaattca ttatggtaaa atatcattcg tatctattgt |
| 297781 | ccgaaatctt gtcaacaaga cagcccaaat tgtgacaata ttccactttt caaataaatg |
| 297841 | agttaacttt gggttggcaa aagttcaatt atagtggaaa ctaatttctt catctaaatt |
| 297901 | aaaaatgtct ctataatgaa ccaaatttac gttggattgg ctaaacaact attcagggag |
| 297961 | tacaaaatgc taaacctagc tataaatgtt gagggcttca ttatagatag gacagtttaa |
| 298021 | aagacagggc tccccttaca tttttaaaat aagtgcacat atatcttctt tttgattttg |
| 298081 | aaataataaa ttgctattta tattgattta tggtgctaat gaattttctc atccaagaag |
| 298141 | agtgaaactt attttgttga aataatcaac taagccatca agtaaaagtg accacaccta |
| 298201 | agttgttccc acctaaaagt agtattttaa aatgcttata aaaactcaga tgcacaaagc |
| 298261 | agagaattcc caagacaagc taaagtaaga aagtaaacag ataaactaaa tttaaataaa |
| 298321 | tgtgtggaat tttcatctca aaaacctata agctagttat aaattttcaa taaaacccta |
| 298381 | ttttaggagg cacaaacaca tcattgcaat tggctttaaa acaccatatc acctacaaag |
| 298441 | agaaaatgat caacatcaga aagagaaaat ttattttaat ttttttcttt cattaagcaa |
| 298501 | aaaacttttt tcatttgtac agtcgtggcc cactgcaacc ttgacctcct gtcattaagc |
| 298561 | aaaaactaat tattcatgac agaagtgatt aatgcaaatg tacagttttt ttgagtagaa |
| 298621 | gaagtgactt taagggttaa ttatttttaa caagttatat ttataggtgt attagtcaat |
| 298681 | gatataagtg ttggggaaag tttattttag aattagaaga aagaagtaaa acaattgtaa |
| 298741 | atattgttta ctctgttgta gtttaaattc actaatgata gtaggtatag ttagttacag |
| 298801 | catcctttct atgaaataat tatgatatgc aattcctaca ataatgggga aagtggcaaa |
| 298861 | atgtgtttag aagttggaaa tagataagtg cagtttcttt atgaaaaggg tttgtctggt |
| 298921 | tgaaagtggc agtgtatttt ctgcctttgg aaaagaagca tgatgtcaaa taaacaggtg |
| 298981 | cagtgtgact ttgttatgga gtaaagactc aaaactgagc agagttctgt tttctttcta |
| 299041 | ggatgggctc cttctttgct cccagcctcc caggcatcaa tatccttcga ctccatacat |
| 299101 | ccatgtactt ccagtgctgg gccgttatgt gctgcaatgt tcctgaggcc agggtcttca |
| 299161 | aagcttccag atcaaataac ttctacctgg gcatgctact gctcatcctc ttcctgtcca |
| 299221 | caatgcctgt cttgtacatg atcgtgtccc tcccaccatc ttttgattgt ggtccattca |
| 299281 | ggtctcttgc ttttgaaatt tgactcaggc atcgtgttct ttcgggggtg gaggtgggaa |
| 299341 | tggtcattca ttgtataagc tattttttcc ccccaaacaa tgacattttt ggttggtgga |
| 299401 | aatgagatcc cggctgggcg cagtggctca tgcctgtaat cccagcactt tgggaggctg |
| 299461 | aggcaggcag attacctgag gtcaggagtt cgagacaagc ctggccaaca cggtgaaacc |
| 299521 | tcgtctctac taaaaataca aagaaaaaaa aattagtcgc gtgtggtggt gggtgcctgt |
| 299581 | aatccctgct acttgagagg ctgaggcagg agaatcactt gagctcagga ggcggaggtt |
| 299641 | gcagtgagct gagatggtgc cattgcactc cagcctggag gacaagagca aaactctgtc |
| 299701 | tcaaaaaaaa aaaaaaagaa agaaatgaga tcctttgggt attcaggttc aataggtgac |
| 299761 | atggagattc ttaggcaagt tctgtctatt tcctcatttg ggaatcataa aagagaaaat |
| 299821 | actttatact tttccaggct ctgcctgggt aaaatatgta tggtgaatct gagctgtttc |
| 299881 | catgttagag gatagctaag agcaaggtct gatgacagtg gtctggatgt gagtttgcct |
| 299941 | gactcbgggc cgtcacatcc agacgtggtc agctatcact acttctcttg tgcattacta |
| 300001 | ccttaaaaag agagagagag agacagacag agagagagag aataccccac actttaccta |
| 300061 | ggaaagagaa ttattactaa atactaatga gcataaggag ggccacccaa tttgttatcc |
| 300121 | aaaatatcac atgctagtga aaaaaattaa ggcbgttcca catatatatt ctctcctaat |
| 300181 | tatcattcaa gtcaggaatc gagagtcaca acggctcctt gatccaattt agagatacac |
| 300241 | atttctacag ttctctatga cagagctctc ttctctctat gacacatatc agaaacctaa |
| 300301 | gtccctggga tctggaggat gaagtttctt tccctaaagg gccttagaaa tgaatctact |
| 300361 | acaagttctc ccaagaatct ctgcatgttg attcactggc cagcatgcac ttgagctgtt |
| 300421 | tctccttttc agtgaaggaa atgagactgt aaaaagccat gtgaatgata tgcatgatgc |
| 300481 | tgtaatgccc acatataatt cctccattat gttgtcagac ccaagttatt taattaaagt |
| 300541 | caaaaggttc attgctgaca gactaagtat ataagagaca ttttcactct taacagtagt |
| 300601 | ctgagttatt gctttgctgg aaaacaagaa aatgttaagc cagctgatcg agttaaacta |
| 300661 | gctggacctc tgtctactca gagaactgat gttgagaagc tctgttaatt ccgttgtgtg |
| 300721 | tgtgtgtgtg tgtgtgtgtg tgtgtgtgtg tgtgtgtgtg tttccaagtg ggctgaaata |
| 300781 | aataatgcag acagtgacat ctggagacta aatatgaaat tacagtcttg atgagacatt |
| 300841 | cgtctcaata aattccgaac caatctcaga gtacaaagtt caaggaatat gccattcaaa |
| 300901 | ggctttagtt aattgcaaat tattcacact acttattatc caggataagt ttaggtgtgc |
| 300961 | attaagctat taattggaag ttgatccttc tcagtaaaac atagcaaagg accctagctg |
| 301021 | cctgtaaggg aaaccacaca atgtaaacat ggatctccac agatgcattc agatttacaa |
| 301081 | tacgtcttag acatgaacaa tagttgcaca atttcagctt cttaagccct aactcttctg |
| 301141 | gctttcctaa ttggcaaact taggcaaaat tatggcatgt aaatcactat tttgaaatca |
| 301201 | gggaaattta ttttttattt ttaaaattat ttatttgttt tttaatttcc ataggttatt |
| 301261 | ggggaacaag tagtgtttgg ttacttgagt cagttcttta gggtgatttg tgagaaatca |
| 301321 | gggaatttta aatattttca atggagtact gggaagggtt atctctatgc tatttagatt |
| 301381 | ttttttttca tgtcagtttc ctcactttag agccaaagga tgtttgcatt gtttatttgt |
| 301441 | ctggacttaa aagtttgaaa ggattgtcct gctctagaaa tgaggtgaga atgcttgtat |
| 301501 | gcttgtacta gtgggtgcct catagcttgt catctttatg aaccccaagt tttaggtgat |
| 301561 | ttgcttaacc ttcctggtga aggttgtctg actctatcta gatcctgggc aagcaggaat |
| 301621 | ttaggaaata taaatttgaa ctaggttact ggaggtatca cagtgatggc tagttgctct |
| 301681 | ccagtgtagg gaagttttca ggtaagatgg ctatagtata tttatccatt cagtaaagca |
| 301741 | atttgatgag atattctagc ttgtctggtt ttataagacc tggctagaga gggtaaattt |
| 301801 | tccaagaaat tattccaata ggctagtaag tggatttatc gtaaatagtt gacaatttaa |
| 301861 | agatgtccag atgaaggcaa ttctaattgt tctatagcat ttcctgacac ctgatgtttc |
| 301921 | ctcccatcta tgtcaataat tctgaacaat ttatagttct ctaggttttt atatacatcc |
| 301981 | ctgcttacat tatgacaatt ttatttttca tatattttca tcttactgca aagataatat |
| 302041 | gtgtttatgc tagaaaacct aaacattgta gaaatatttt tacatgaaaa gaaacatggc |
| 302101 | ccataatctt gaaaacctga aataacaact ttttagcatt ttatattctg ttttgccttt |
| 302161 | catgcatgat ttttcataac tgagcttgta ctgacacagg atgtttctca gcctcttcac |
| 302221 | tggactcaca gaggggcacc ctgtctactc ggcctgctgt gcttaacccc ttgtgggagg |
| 302281 | aagcacgtgg gcaagtgagt gcgggatctg gcgagacaca ctgggcactg acacaggagc |
| 302341 | aagctccatg tggggcccgt ggccagacca ggtgtgtcac cttgaggaga atgcaatggt |
| 302401 | gcccaagtgc ctatgaccct aaagccccag agggggtgtt acaatgcttc ctgagttcca |
| 302461 | ccatcctcag cctgatggat ggcagtgtct tagcagctca gttggcccct tgcctcatca |
| 302521 | tgtggggcag ctgccctcca ctggagaggg caaagggcca gtgtgacagt ctttcctggg |
| 302581 | ttcccacaat cggtgggtcc caagctcttg tccagcatcc aagaagaatg aggtcacatg |
| 302641 | gattattgaa ggatggtgaa ggaggagaat tttattgagt aatgaaattg gctctcagca |
| 302701 | gagagggcag ttggagaggg gccaggaagg gcaggtcatc ttccccaaag tcaggttgtc |
| 302761 | tcttctccaa agtcaggccg actcttcccc aaagtcaggc cgtctctccc ctctaccaac |
| 302821 | tgagtctgtg gtctttatag gcacaggatg gaagtgtgtg ctgactggtt tgtgagtatg |
| 302881 | caaaaaaggc taaagtgaag gtgtcactca aaggtagaca cgacagtata gaaacccaac |
| 302941 | taggaaaggg taggtatatg taaaataggt gaagggtggg gatgaatcag aggaaagtgc |
| 303001 | gccaaacagg aagacaagtt ctcaatccgg tctgaggatt taacttggac cttggctttc |
| 303061 | aggctttaaa ctgtctttgg cttggaggtg gcatttcacc aggtacctgc ccctatctgc |
| 303121 | ctaggcattt ggctgcctcc tgttgctatc agtatactag gtgcaggttt tttagtatta |
| 303181 | aaagttaaaa ttatattgtg agactgctat tatattggtc atttgcaatc atgctttatg |
| 303241 | catctatttt tcttaatagt ttagctgact tctagagatt ggggatagag aagtttcctc |
| 303301 | tctgtggatc taggacacag taaagtgctg gtcaataatt tgaatgaatg aatgaataat |
| 303361 | gactgcttat tgtgcactaa gcactagaag agacacaggg ttaagtccat gcattaagca |
| 303421 | ttgattctgg tcctgtggca cttatcaccc agcbgggaag acaggcctcc tcataaactg |
| 303481 | ttctaatcta gagtggcgct atgcctacag caggactatg gatagacaca gacaactgga |
| 303541 | aaacaattct gagaatattg tatgatagca tactttggct aacctggcag gagaaggcat |
| 303601 | tgccagggac atgtagatgc gtccataatt tgtacgtaat taagatatct ggactccttt |
| 303661 | ctttggtaaa agacctgacc tgaaaaaggt acacaggaat ccccagggac ttagcaactt |
| 303721 | tgctttgggt cacactggat gctgagaagg cctttgtgtg tagggataat ttcataaaat |
| 303781 | ttgaatgccc ccactctaac tacgttcatc agctatgacc tgaggtgctg gagaagggag |
| 303841 | gatagatggt cttatctttt ttggtacttt taatttatct gcttatcctg tattccatct |
| 303901 | ggcbggggct gcattggggg gtatgaacaa tttggaatgg tgaaaaataa aacatattgg |
| 303961 | aatagggagt tataatctta tattgcttgt aagcttcatt ccctcttatg tatcataagt |
| 304021 | cttgaaaatt agagtgtaga tctttaattt gtgaagcaaa acttgtacct ctgtagtcca |
| 304081 | atgactactg acaaaatata tggagtcact atgtctgctg gaagatagta ttggttagac |
| 304141 | caggagaaaa aaaccataat ctgggacttg cgtgtcattg ttatttagta gttgaaaatg |
| 304201 | cttaaagatt aaaatacaaa agcaaaacta aacattgtat taaatgttgc accaacattt |
| 304261 | attctgatga tgcatacaat ttggtgaaac ccaacccaaa tctcttctga aaattacatc |
| 304321 | ttttcgtagt acaatcagta tgcatataca gagaaaataa tgatgtaatg aatattttga |
| 304381 | atattatggt acaattctag ctattcctta gtaagtcaag acatcctgtt ctcagttttc |
| 304441 | tagtttatag gagctgatac ttgccttcat ggagcttagc agtggagcta agctggtatt |
| 304501 | cattagggcc tgtccaggag aagaaactga gagggaaaaa acccatgatt cggtgtggtt |
| 304561 | accttaccta ttaacatatc tgatgaagga agtctggtgg aaaatgaggt ggaagctatt |
| 304621 | gcgtggaaag agatattgga ttatatgtgg attttattcc actgggctat tccttgctct |
| 304681 | catttccaag gctaattaaa aatacttagc tcaaagagag tgttctccta caagagcagc |
| 304741 | tgactgagag gcagacttat gagctattta caaaatatat ttcaatcaaa atcttagaaa |
| 304801 | tacagttttt aaaaatcttc tcctaaatat gtggttaatt ttctaactgc tttgactata |
| 304861 | tgtttatact gggatcatat ttctcgtatc ttacccatat attgactaca tgaggtcaaa |
| 304921 | gtgtcagcaa gttgtagcta aacatcagat tcctaccctg aaagaggacc taagcagtaa |
| 304981 | ttgagaaaga tttccagttt tcttgcttta accatggttt ttccatatgt tcttaataaa |
| 305041 | cttatctccc cctttttaat tcccccagtg gcaaaaatag aatgtttgaa gtcattggag |
| 305101 | agaccctgga gcacgatttc ccaagctgga tggcgaagat cttgagacag ctttcaaacc |
| 305161 | ctgggctggt cattgcbgtc attttggtga tggtgtatgt gcttttcctc ctcatagaaa |
| 305221 | gagcctctgt gatgttcttc ctggctgttt ttacatttca gctttttcag ctctctcact |
| 305281 | ctccctaagg gttctgctgt aacctatcca aattcaacac tgattacatg gtggtgagag |
| 305341 | gctaaaattc atgagccata ttctgatcct attggatgta attcacaggc atctgcatct |
| 305401 | gtctttgctt ccccaggatt ataagatttg ttcccaagta accacaataa ttatccataa |
| 305461 | acagctaaag caagggaaac ataaatgcta aatgctaaat ctaatcagta cattgagctt |
| 305521 | ccccccaaca acccggcctc cacctctcgt tccataaagc agatggcaat gaatatacag |
| 305581 | aaataatgat cctctaagtt aatattcccc ttgggggccc tatagactat attttagggg |
| 305641 | caagggcttc aagggtctta aaaatttcac tgtccttgtt gttagagggc cgccaataac |
| 305701 | tgtgtgttca cgataataga atcagaggac cagaggatct tgggaagttc ctgtagtctg |
| 305761 | tcccttcaaa gtcagaagtg atttccaaag gggaacttgt cagatgactt ttgttttcac |
| 305821 | ataactttca ttggcttctg actcttggcc atcacagata aacttagatt ctaagtaaaa |
| 305881 | gtacagattt gcbgtggaag agccaaaagc agcgtccatc tcttcctttc agtccctgag |
| 305941 | tgcctcctac atagaatgtc tggagctgcc acgactctca gtcagtgggt aaatattaaa |
| 306001 | cagccccagg gacactttaa gaacaaagtt gatgcaaagg atttggttca tttaaataaa |
| 306061 | ggtaaaacaa ggaaatctca gggagaaaac tcccagtggt ttgcagtaaa tgagcaggga |
| 306121 | ttgtgaccca gttattgtcg tgatcacaag ccacataaag tagtgagggc aggtttatct |
| 306181 | tcttcaattt tataacctaa gaaattgatt tacacaaaga aagtgatttt ccctagacaa |
| 306241 | gagagagagt aaaatggtca tagaaggacc tgaagcagat cctttcacag taaatccagt |
| 306301 | gctcacttct ttatactgcg aggctttctg tgagggagag ctaataacct gtagggataa |
| 306361 | ttaaacagta aatcagatta ccattatata ttacataacc ttgataatgg ggtgctatta |
| 306421 | agaatctgat agtaaatctc tggcacagat gcattagaaa atgacctact gaatgcagca |
| 306481 | tgtttcacca gatggccttt gaaacaattt tctttctcta catgtccatg atacagtggc |
| 306541 | ttctgagagc atgttaatca gtatcctgtg gcctttgaga gtcaaggccc tttagtggta |
| 306601 | tgaatgaggt gttgtttaaa acacacacac acacacacac acaatgtaaa tgtcagattt |
| 306661 | cgttaccata ttcttacttg attaggagac atgtttattt ttaagtgaag gagtggcatt |
| 306721 | gttcaaatgc cttaatccaa agatttagta atcagtccta agagggttta aaaggaattc |
| 306781 | acactgcctt tgtttcaggt actatttaca cactgactca tttaattccc ataacagctt |
| 306841 | tgagaagctg gcttcattaa tcatactatc aataacacat tgaaattctg ccccaagtca |
| 306901 | agtgactaag tggaactgaa actcatgtct tctccttatt tcctacactc acttgcagca |
| 306961 | ccaaacaggg atcagaatca gtgggaattt cagatgggct gaaagccact tgtccgaggt |
| 307021 | ggtatagacc cagggtcagc acacttttcc taattcgagg gccagaggca aattaggcca |
| 307081 | agaggcaaaa ttgaggctct taattaagta attactaata ataaccgttt aaaagacaga |
| 307141 | aaaaccattc ttaattcatg ggccatacaa aacagtctgg cagtcatctt catcctgcag |
| 307201 | gacctagttt gctttgatct ggacagcctg gaggaaagct ggccccatgc aaatgtcaaa |
| 307261 | tagatgtact gaaaggttga ttgtggctct gaggttctga tttaaaaccc agattttagc |
| 307321 | ttattactga gtatattttt tcaaattctg agatctcatc ttctattttt ggggcccata |
| 307381 | caattggctt agctttttat tacctttaat tagagaattg tactagtatg ttgaatttcc |
| 307441 | cccaatggat tatattaaat caacctggat ggctttcggg tatctaagca gagttcaggt |
| 307501 | tagttctctg caggcagttt cattcctttg gagactagaa aaagatttgt gaggaatact |
| 307561 | tatttaacgt gcattacaag gtagaaaatc atggtggata aatagagaat tccagaactt |
| 307621 | gaaaggactt gtgaaatctt tgagttcatc ttccaaatcc aggaactttt ctaagaaaat |
| 307681 | ccttataata cacaccacac acaaacacac acacacacaa acatttattg agcacctatt |
| 307741 | tcatgccaga tattgccctg catgcattta tgtatgtaat tttagtaaaa ctcccatcac |
| 307801 | tcatgtaaga gataaacaaa ctcagccaaa ccgtatcaat aattgtgttg ctagcaccag |
| 307861 | atggttcata aaggatactg tctttcattt tggggagtag aggaaatgtg ttcaatatcc |
| 307921 | agttatttgc agcaaagagt aatataagtt acctttttta ctgcatttct tctttgtttg |
| 307981 | tttgttcgtt ttgccttaag tgaagataag catatttatt tttacagaat tgtctggcta |
| 308041 | gaatttgccc tttctagaat gtaaatgtca tttaaaatcc aataatattc ctttatacaa |
| 308101 | cgttttcttt ttgagggaga aagaaatgca aacaatacat tctacggaaa aaattccatg |
| 308161 | ttcattatct atgcctatga ggattacatt cttagctgca aaggccaaag gacgaaattt |
| 308221 | aaggcagaaa gtcgttttcc tttttgttcg gcatgttccc taagctgcag taggttttcc |
| 308281 | attctctggg atgccattgc catccattta aaatgctact ttcaattata atttaaactt |
| 308341 | atgtgaatac atttccagga aaaagctaat attaggcagt gtgaaatagc agacagaaca |
| 308401 | ggctgtggat caggagcctt gggccccaac tttccaactt caccccagct tcatccctat |
| 308461 | gtcatcctga atgtatctct ttacctctct ggacctcagc ttttatctat aagacaagag |
| 308521 | atttagagta gaagatagct taatgttcat tcccatgctg atatttcata atttacttaa |
| 308581 | tgtaaattta agaagtatct tggggaactg aaatatacct tacactgtat tttttttctt |
| 308641 | tttaatttag tttggccatc tattatctca atgctactgc caagggccag aaggcagcga |
| 308701 | atctggatct caaaaagaag atgaaaatgg tatgatacaa tttatttcat agaaatatta |
| 308761 | tctttattaa tgtcaagcag tagaaaacac tgagaaatct acttttttcc ccttattttt |
| 308821 | tattgtgtag caagctttgg agaacaaaat gcgaaacaag aaaatggcag ctgcacgagc |
| 308881 | aggttggaga tacgtttatg tttgtaatgt ttgtaatttt tctttttctt tttctttctt |
| 308941 | tttttttttt ttttgctttt tctccattgg atggtgagtg gcattattgc tgtgagcgag |
| 309001 | tcattccaca atccttacct tccacctttt tagcccttct caggcatcac atccagggta |
| 309061 | tttggttgtt ttctagtggg aggaaaaaaa gagaaatgaa aatgtgggca tataaacttg |
| 309121 | gcagtagtga tttttaaaag tgaccttgaa aatgcgcaga aaatgaagaa atcccccata |
| 309181 | tagtcgtcta caaacagcag atacaaatgc agatgctctc ctgcccatgt tgactatttt |
| 309241 | catacatgga aggatatggc aagaagatcc caagagggtc ctctgggctt tccaccactg |
| 309301 | attgccagaa gccctttcta atccattaaa ctttcttttt ttaaaaacct ctttattttt |
| 309361 | taaagtatta gagtaaagcc aacttctagt tgccaaaatt aataatatgc tttgagttcc |
| 309421 | ccacggaaag gtgataccta agacctttag tgaaaatctt ttccaacttt gtccttactc |
| 309481 | ctaactttaa aactctttac attagtagga ctgtttcccc actgtaagta ataaaacact |
| 309541 | caaattggtt taattgagaa agagtaattt attcgaccac acaaatgcag ggaaatggta |
| 309601 | tgtatggctt caggccactt tcttttttct atatatatat atatttttta attatacttt |
| 309661 | aagttctagg gtacatgtgc acaacgtgca ggttagttac atatgtatac atgtgccatg |
| 309721 | ttggtgtgct gcacccatta actcatcatt tacattaggt atatgtccta atgctagccc |
| 309781 | tccctgctcc ccccaccgca cgacaggccc cggtatgtga tgttcccctt cctgtgtcca |
| 309841 | agtgttctca ttgttcaatt tccacctatg agtaagaaca tgcggtgttt ggttttttgt |
| 309901 | ccttgcgata gtttgctgag aatgatggtt tccagcttca tctatgtccc tacaaaggac |
| 309961 | atgaactcat cattttttat ggctgcatag tattccatgg tgtatatgtg ccacattttc |
| 310021 | ttaatccagt ctatcattgt tggacatttg ggttggttcc aagtctttgc tattgtgaat |
| 310081 | agtgccgcaa taaacatacg tgtgcatgtg tctttatagc agcatgattt ataatccttt |
| 310141 | gggtatatac ccaaaggatt ttttttcttt tttgagacct ggtctggctc tgtggcccag |
| 310201 | gctggagggc agtggtatga tctcggctca ctgcagctcc aacttcccag gctgaagtga |
| 310261 | tccttcctcc tcagcctcct gagtagtggg gacagcgcac accaccatgc ctggataatt |
| 310321 | tttaattttt tttttgaagt gaggtcttgc taagttgccc aggctgctct caaagtcttg |
| 310381 | ggctcaggtg atcttctctt tttggcctcc caaagtgttg ggattaaaag catgaaccac |
| 310441 | tgtgcccagc attcagggca ctttttatcc taagtttaga acagagtcag gtctgagccc |
| 310501 | acctctctgt gaatctctga agctaaactc ctttcacatt ggctgacctt tatggcagca |
| 310561 | ggaatggatg cagcaacttc caatgccatg tccttagact gtaaaacttc tttcctagaa |
| 310621 | attcctcaac ctctcctcct ttcagattgg ccccatatgg gtcatgtatc catccttgtg |
| 310681 | ccagtcactg tgtctgagta tggtaagtta ctgaagtcag tcaggaccca tccttgaagc |
| 310741 | tgggattggt ctgttttcct gacacatgtg aaatgcactg gggaggtcac acctacctaa |
| 310801 | gagaaggaga aaaggatgct gggtgtcccc aaacaacaaa tgttcaagac ttggttttgt |
| 310861 | tgccttcttt atgcttagcc ttttttcttt tccctctctc tatctagcat gcataaatgt |
| 310921 | aaagttgaat gcbgggtgct gaattagatg attacagtga tgtacagtta tcagactagt |
| 310981 | ttttaatgaa atttttaatt ttttaaatca atactacatg tgtgcataat ttagagttcc |
| 311041 | atttagaatt tcataattct ataaggctta tcatgaaaaa cagcatcttc ttctgcctcc |
| 311101 | tacaccagtt ttgctctctt ctcctcccct gcactcaaat accctaagac aaacactttc |
| 311161 | aatttgttag ttgattttta gatatttatc tccaaattac ctaattttgt tgtaatttct |
| 311221 | tgacttttta gttttaagta ttatctctta aaattattaa ctacaatgga tataataatt |
| 311281 | ttaatagata atatttaaac gatttttata ctcttgcccc agtctttcta tgcattctac |
| 311341 | ctatgtagtt atattattat tttggttagg ccagtataca ttattatgga cacacagtct |
| 311401 | tcacagctaa gccaagtagt aaactatgat acttgtgcat actttaaaat tttctcagga |
| 311461 | ttcataattc ataatttctc tttttaaaca ttttcttatt tttatatatg tttcagtgat |
| 311521 | gcatttaatt tccaaatctc tgctatttat tgagttatac tctcaatata ttcagacata |
| 311581 | ttgggttatc tgtccatttc atttccttag agaaatcttt cccagaatct tctgcacttc |
| 311641 | tccaatttca actagtgtgt gtctgggata cagctgtcat cctttcattt ttcttcactg |
| 311701 | tcattcaaca ggatctctgt cactttatcc tgttttggat actgtgtttt ccattttccc |
| 311761 | caacttcctc tcttttggtc tgctcacatt ttgccagtaa gaaagggtat gtgagaggca |
| 311821 | aattttctga gaccatgcct atctgaaatt gttcttctct tactctccct tttgactgat |
| 311881 | agtttggggg tcaaaaatac tcatctaatt atattagaaa taatatccct tcaaaatagt |
| 311941 | attttttcag aggtttccca tgccttttag attccacaat tgctcttgag tagtttaaag |
| 312001 | ccattgtgat tcttagtctt tcttgagcaa cttgtatttt tgattatctg tttggttttc |
| 312061 | caatctggaa agttctaggg tctccttatc tgcatgttca gaatttcatg atggtgtatc |
| 312121 | ttgcatggat ctatttttat tccttgtgta ctcatcaatg gacttttgct ctttggttga |
| 312181 | agggaatttt cttgaattgt ttacaaattc tttccatcca ttttctgttt tttttttttt |
| 312241 | catgtagaat ttctaattta tttttcatcc ttaaattccc tttgtctctt tgctttacct |
| 312301 | atgaacattt ccttatcttt aaattttaac atttctcttg gccttttcac ttttgctatc |
| 312361 | ataattttag tttctgcaag ctgttttgtt ctttaattct tcttttttaa acagcatgct |
| 312421 | gtttttgttt aatggctgca atggcttgtt ttgtctttat gagaatatta ataactcttt |
| 312481 | ttgatgtttc tttgcctttg atagtctgtt ttctctaagc tgttttgttc ttctttctcc |
| 312541 | cctgcttggt aattcccctg ctttgtgacc ctcagctcat atttcagggt aggatgcaaa |
| 312601 | ggtgggtttc gaagctttaa gcacatggat ggggcttgtg ggcagatcct cattggggga |
| 312661 | ctctgcctgg gctgccgctg aacccccatg tcagtatttt taggcttctc ctcttaagct |
| 312721 | gatcagattc ctcagaaaaa ttacttccga actcctgccc agagagtaaa gatttgcctg |
| 312781 | gcaaatctgt agcacctgag tgggaaaaac aacacgggtg gggctcagct ttcaacctac |
| 312841 | aaatgctcac ttagccccct ctttttactg cggcactcct tgtggtcttc taacccagaa |
| 312901 | gcatctctgt tttactcttt ttagaaaaaa ttattccagt cttctgccag attaaaggta |
| 312961 | gtgggggttg gggagcaatt attcatttgt accaagtgaa gccgatctca gactctaact |
| 313021 | gctgctgttt cgttttttaa taacccccac ccctttgccc caccaagctt gactgcttct |
| 313081 | taacagctaa aacatcctct ttgtattggt tccttacttc cagaggtacc tgttgctgca |
| 313141 | aattactgaa cgtttcagag attttatgtg taaattatgt tgcttactgt ctttttcctc |
| 313201 | ccctggctta gcttttcagt ttgttagaat tgataagtca ttatcattaa tctgtctgct |
| 313261 | ttccagcttc taaaattttg tcattgatgt ctcttcccct attctccccc tccttgcagg |
| 313321 | tttatgcaac cttctccatc ttcctcccca accccctcaa aatttcttga ctgtcatgtt |
| 313381 | tgtgaaaggg tgaaagttca attcaatgta tttacctccg cagtttaggc aaacctttgt |
| 313441 | atttcctaca ctaggagaac aatggtttta tgtccatctt cactctgttc atcccacagt |
| 313501 | gcttacctga ggtattattt tgaaatagag tctgggatgg agtctacctt agtgagaatt |
| 313561 | tattagaatt gtttagtctc tgcagaagat gaggtggttg aagcacatac atcacatata |
| 313621 | ctagcagtgc ttttgtataa tttaatatta tcaaattagt acttaagaat ggaaaattct |
| 313681 | gacttataag atctaaattc taattaaggc ttattgttat ctttattgtt ttaaatgata |
| 313741 | atataatgct atagcatgct attcatctga tattttttcc ataatgtgta tggccaccca |
| 313801 | gtaaacacct actgaatatc tactgaaaaa tgaggaactc ttttgttgga aaaatataaa |
| 313861 | atattcatca cataagcaat aatgtataaa tgttacaaaa aagtagtaca ttcattctgc |
| 313921 | cttgtttaga atatgtgaag cctttgcaaa ctaagaaaca tggaaatatt ttgaacatta |
| 313981 | aaatggtaaa tattatggga aagtagagca catttggaca atacagattt ctggccacct |
| 314041 | cattcagaac cattaagcac actgaaagct acgtttttat ttgcctcctg ttcatcttct |
| 314101 | ctctctctct ccttgttttt tttttttttt tttttctgtt ttgtgaacag ctgcagctgc |
| 314161 | tggtcgccag taataagtat cctgagagcc cagaaaaggt acactttgcc ttgctgttta |
| 314221 | aaagtaatgc aatatgtgaa cgcccagaga acaagcactg tggaactgct attttcctgt |
| 314281 | tctacccttg atggattttc aaggtcatgc tggccaatta aggcatcatc agtcctacct |
| 314341 | gagcaacaag aatctaaact ttattccaag tcagaaactg tttctgcaga gccactctct |
| 314401 | cccctgctcc atttcgtgac tttttttttt tttttaacaa attgagttta gaagtgagtg |
| 314461 | taatccagca atacagttta ctggtttagt tggtgggtta attaaaaaaa atttgctcat |
| 314521 | atgaactttc attttatatg tttcttttgc c |

### Pejvakin (PJVK)

The PJVK gene encodes “pejvakin” (PJVK). PJVK is a member of the gasdermin family, and is required for the proper function of auditory pathway neurons. Mutations in PJVK may lead to non-syndromic sensorineural hearing loss. For example, a v.930_931del AC frameshift mutation in PJVK has been associated with deafness (Zhang et al., Acta Otolaryngol 135(3): 211-216, 2015).

The human PJVK gene is located on chromosome 2q231. It contains 8 exons encompassing ˜17 kilobases (kb) (NCBI Accession No. NG_012186.1). The full-length wildtype PJVK protein expressed from the human PJVK gene is 352 amino acids in length.

An exemplary human wildtype PJVK protein is or includes the sequence of SEQ ID NO: 51, SEQ ID NO: 53, SEQ ID NO: 55, SEQ ID NO: 57, SEQ ID NO: 59, or SEQ ID NO: 61. Non-limiting examples of nucleic acid encoding a wildtype PJVK protein are or include SEQ ID NO: 52, SEQ ID NO: 54, SEQ ID NO: 56, SEQ ID NO: 58, SEQ ID NO: 60 and SEQ ID NO: 62.

Human PJVK Protein Isoform 1 (NCBI Accession No. NP_001036167.1)
(SEQ ID NO: 51)
MFAAATKSFVKQVGDGGRLVPVPSLSEADKYQPLSLVVKKKRCFLFPRYKFTSTPFTLKDILLGDREISAG
ISSYQLLNYEDESDVSLYGRRGNHIVNDVGINVAGSDSIAVKASFGIVTKHEVEVSTLLKEITTRKINFDH
SLIRQSRSSRKAVLCVVMESIRTTRQCSLSVHAGIRGEAMRFHFMDEQNPKGRDKAIVFPAHTTIAFSVFE
LFIYLDGAFDLCVTSVSKGGFEREETATFALLYRLRNILFERNRRVMDVISRSQLYLDDLFSDYYDKPLSM
TDISLKEGTHIRVNLLNHNIPKGPCILCGMGNFKRETVYGCFQCSVDGQKYVRLHAVPCFDIWHKRMK
Human PJVK Isoform 1 cDNA
(SEQ ID NO: 52)
atgtttgctgctgctaccaagagctttgtcaagcaagttggagatggagggagattagttcctgttccaag
cctcagtgaagctgacaaatatcaacctctaagtctggtggtaaaaaagaagcgatgctttctgtttccta
gatataaatttacttcaacaccttttacactgaaagatattctcctaggagacagagaaatttcagctggt
atttcatcttatcaattactgaattatgaagatgaatcagatgtttcactctatggaaggcgaggtaacca
tattgtaaatgacgttgggattaacgttgctggatcagattccattgcagtgaaagottcatttggtatag
taaccaaacatgaagtggaagtatcaacattactcaaagaaattactacacgaaaaattaactttgaccac
agottgatacgtcagtcaaggagcagcagaaaggcagtattgtgtgtggtcatggagagcatccgaaccac
acgacagtgctcactgtctgtgcatgctggaattcgaggggaagcaatgoggtttcactttatggatgaac
agaatcccaagggaagggacaaagctattgttttcccagcacatacaaccatagotttcagtgtttttgaa
ctottcatatacctggatggtgcctttgacctttgtgtcacttcagtgtcaaaaggaggatttgaaaggga
agaaacggcaacatttgcactgctgtacaggttgagaaatatcctatttgaaagaaatagaagagtgatgg
atgtcatttctcgttcacagotttacttggatgatcttttttctgactactatgacaaacctctcagcatg
actgatatttcactcaaagaagggacccatatccgagttaacttacttaatcacaacattcccaaagggcc
ttgcatactctgtggaatggggaacttcaaaagggagacagtttatgggtgotttcagtgttctgttgatg
gtcagaagtatgtgagacttcatgcagttccttgttttgatatttggcacaagaggatgaaataa
Human PJIVK Protein Isoform 2 (NCBI Accession No. NP_001340704.1)
(SEQ ID NO: 53)
MAFAWDAAPLSFVKQVGDGGRLVPVPSLSEADKYQPLSLVVKKKRCFLFPRYKFTSTPFTLKDILLGDREI
SAGISSYQLLNYEDESDVSLYGRRGNHIVNDVGINVAGSDSIAVKASFGIVTKHEVEVSTLLKEITTRKIN
FDHSLIRQSRSSRKAVLCVVMESIRTTRQCSLSVHAGIRGEAMRFHFMDEQNPKGRDKAIVFPAHTTIAFS
VFELFIYLDGAFDLCVTSVSKGGFEREETATFALLYRLRNILFERNRRVMDVISRSQLYLDDLFSDYYDKP
LSMTDISLKEGTHIRVNLLNHNIPKGPCILCGMGNFKRETVYGCFQCSVDGQKYVRLHAVPCFDIWHKRMK
Human PJVK Isoform 2 cDNA
(SEQ ID NO: 54)
atggcgtttgcctgggatgcagcacctttgagotttgtcaagcaagttggagatggagggagattagttcc
tgttccaagcctcagtgaagctgacaaatatcaacctctaagtctggtggtaaaaaagaagcgatgctttc
tgtttcctagatataaatttacttcaacaccttttacactgaaagatattctcctaggagacagagaaatt
tcagctggtatttcatcttatcaattactgaattatgaagatgaatcagatgtttcactctatggaaggcg
aggtaaccatattgtaaatgacgttgggattaacgttgctggatcagattccattgcagtgaaagcttcat
ttggtatagtaaccaaacatgaagtggaagtatcaacattactcaaagaaattactacacgaaaaattaac
tttgaccacagottgatacgtcagtcaaggagcagcagaaaggcagtattgtgtgtggtcatggagagcat
ccgaaccacacgacagtgctcactgtctgtgcatgctggaattcgaggggaagcaatgcggtttcacttta
tggatgaacagaatcccaagggaagggacaaagctattgttttcccagcacatacaaccatagctttcagt
gtttttgaactcttcatatacctggatggtgcctttgacctttgtgtcacttcagtgtcaaaaggaggatt
tgaaagggaagaaacggcaacatttgcactgctgtacaggttgagaaatatcctatttgaaagaaatagaa
gagtgatggatgtcatttctcgttcacagctttacttggatgatcttttttctgactactatgacaaacct
ctcagcatgactgatatttcactcaaagaagggacccatatccgagttaacttacttaatcacaacattcc
caaagggccttgcatactctgtggaatggggaacttcaaaagggagacagtttatgggtgctttcagtgtt
ctgttgatggtcagaagtatgtgagacttcatgcagttccttgttttgatatttggcacaagaggatgaaa
taa
Human PJVK Protein Isoform 3 (NCBI Accession No. NP_001340705.1)
(SEQ ID NO: 55)
MYVYRLLSITVLMDILLEGLHSQYFTIYELRITPMAKHFSTQSFVKQVGDGGRLVPVPSLSEADKYQPLSL
VVKKKRCFLFPRYKFTSTPFTLKDILLGDREISAGISSYQLLNYEDESDVSLYGRRGNHIVNDVGINVAGS
DSIAVKASFGIVTKHEVEVSTLLKEITTRKINFDHSLIRQSRSSRKAVLCVVMESIRTTRQCSLSVHAGIR
GEAMRFHFMDEQNPKGRDKAIVFPAHTTIAFSVFELFIYLDGAFDRRVMDVISRSQLYLDDLFSDYYDKPL
SMTDISLKEGTHIRVNLLNHNIPKGPCILCGMGNFKRETVYGCFQCSVDGQKYVRLHAVPCFDIWHKRMK
Human PJVK Isoform 3 cDNA
(SEQ ID NO: 56)
atgtatgtatatagattactatccatcactgttttaatggatatcttattggaagggctacatagtcagta
tttcacaatatatgaactccgcataacacctatggcaaaacatttcagcactcagagotttgtcaagcaag
ttggagatggagggagattagttcctgttccaagcctcagtgaagctgacaaatatcaacctctaagtctg
gtggtaaaaaagaagcgatgotttctgtttcctagatataaatttacttcaacaccttttacactgaaaga
tattctcctaggagacagagaaatttcagctggtatttcatcttatcaattactgaattatgaagatgaat
cagatgtttcactctatggaaggcgaggtaaccatattgtaaatgacgttgggattaacgttgctggatca
gattccattgcagtgaaagottcatttggtatagtaaccaaacatgaagtggaagtatcaacattactcaa
agaaattactacacgaaaaattaactttgaccacagottgatacgtcagtcaaggagcagcagaaaggcag
tattgtgtgtggtcatggagagcatccgaaccacacgacagtgctcactgtctgtgcatgctggaattcga
ggggaagcaatgoggtttcactttatggatgaacagaatcccaagggaagggacaaagctattgttttccc
agcacatacaaccatagotttcagtgtttttgaactottcatatacctggatggtgcctttgatagaagag
tgatggatgtcatttctcgttcacagotttacttggatgatcttttttctgactactatgacaaacctctc
agcatgactgatatttcactcaaagaagggacccatatccgagttaacttacttaatcacaacattcccaa
agggccttgcatactctgtggaatggggaacttcaaaagggagacagtttatgggtgotttcagtgttctg
ttgatggtcagaagtatgtgagacttcatgcagttccttgttttgatatttggcacaagaggatgaaataa
Human PJVK Protein Isoform 4 (NCBI Accession No. NP_001340706.1)
(SEQ ID NO: 57)
MESIRTTRQCSLSVHAGIRGEAMRFHFMDEQNPKGRDKAIVFPAHTTIAFSVFELFIYLDGAFDLCVTSVS
KGGFEREETATFALLYRLRNILFERNRRVMDVISRSQLYLDDLFSDYYDKPLSMTDISLKEGTHIRVNLLN
HNIPKGPCILCGMGNFKRETVYGCFQCSVDGQKYVRLHAVPCFDIWHKRMK
Human PJVK Isoform 4 cDNA
(SEQ ID NO: 58)
atggagagcatccgaaccacacgacagtgctcactgtctgtgcatgctggaattcgaggggaagcaatgcg
gtttcactttatggatgaacagaatcccaagggaagggacaaagctattgttttcccagcacatacaacca
tagotttcagtgtttttgaactottcatatacctggatggtgcctttgacctttgtgtcacttcagtgtca
aaaggaggatttgaaagggaagaaacggcaacatttgcactgctgtacaggttgagaaatatcctatttga
aagaaatagaagagtgatggatgtcatttctcgttcacagotttacttggatgatcttttttctgactact
atgacaaacctctcagcatgactgatatttcactcaaagaagggacccatatccgagttaacttacttaat
cacaacattcccaaagggccttgcatactctgtggaatggggaacttcaaaagggagacagtttatgggtg
ctttcagtgttctgttgatggtcagaagtatgtgagacttcatgcagttccttgttttgatatttggcaca
agaggatgaaataa
Human PJVK Protein Isoform 5 (NCBI Accession No. NP_001340707.1)
(SEQ ID NO: 59)
MESIRTTRQCSLSVHAGIRGEAMRFHFMDEQNPKGRDKAIVFPAHTTIAFSVFELFIYLDGAFDLCVTSVS
KGGFEREETATFALLYRLRNILFERNRRVMDVISRSQLYLDDLFSDYYDKPLSMTDISLKEGTHIRVNLLN
HNIPKGPCILCGMGNFKRETVYGCFQCSVDGQKYVRLHAVPCFDIWHKRMK
Human PJVK Isoform 5 cDNA
(SEQ ID NO: 60)
atggagagcatccgaaccacacgacagtgctcactgtctgtgcatgctggaattcgaggggaagcaatgcg
gtttcactttatggatgaacagaatcccaagggaagggacaaagctattgttttcccagcacatacaacca
tagotttcagtgtttttgaactottcatatacctggatggtgcctttgacctttgtgtcacttcagtgtca
aaaggaggatttgaaagggaagaaacggcaacatttgcactgctgtacaggttgagaaatatcctatttga
aagaaatagaagagtgatggatgtcatttctcgttcacagotttacttggatgatcttttttctgactact
atgacaaacctctcagcatgactgatatttcactcaaagaagggacccatatccgagttaacttacttaat
cacaacattcccaaagggccttgcatactctgtggaatggggaacttcaaaagggagacagtttatgggtg
ctttcagtgttctgttgatggtcagaagtatgtgagacttcatgcagttccttgttttgatatttggcaca
agaggatgaaataa
Human PJVK Protein Isoform 6 (NCBI Accession No. NP_001356841.1)
(SEQ ID NO: 61)
MFAAATKSFVKQVGDGGRLVPVPSLSEADKYQPLSLVVKKKRCFLFPRYKFTSTPFTLKDILLGDREISAG
ISSYQLLNYEDESDVSLYGRRGNHIVNDVGINVAGSDSIAVKASFGIVTKHEVEVSTLLKEITTRKINFDH
SLIRQSRSSRKAVLCVVMESIRTTRQCSLSVHAGIRGEAMRFHFMDEQNPKGRDKAIVFPAHTTIAFSVFE
LFIYLDGAFDLCVTSVSKGGFEREETATFALLYRLRNILFERNRRVMDVISRSQLYLDDLFSDYYDKPLSM
TDISLKEGTHIRVNLLNHNIPKGPCILCGMGNFKRETVYGCFQCSVDGQKYVRLHAVPCFDIWHKRMK
Human PJVK Isoform 6 cDNA
(SEQ ID NO: 62)
atgtttgctgctgctaccaagagctttgtcaagcaagttggagatggagggagattagttcctgttccaag
cctcagtgaagctgacaaatatcaacctctaagtctggtggtaaaaaagaagcgatgctttctgtttccta
gatataaatttacttcaacaccttttacactgaaagatattctcctaggagacagagaaatttcagctggt
atttcatcttatcaattactgaattatgaagatgaatcagatgtttcactctatggaaggcgaggtaacca
tattgtaaatgacgttgggattaacgttgctggatcagattccattgcagtgaaagottcatttggtatag
taaccaaacatgaagtggaagtatcaacattactcaaagaaattactacacgaaaaattaactttgaccac
agottgatacgtcagtcaaggagcagcagaaaggcagtattgtgtgtggtcatggagagcatccgaaccac
acgacagtgctcactgtctgtgcatgctggaattcgaggggaagcaatgoggtttcactttatggatgaac
agaatcccaagggaagggacaaagctattgttttcccagcacatacaaccatagotttcagtgtttttgaa
ctottcatatacctggatggtgcctttgacctttgtgtcacttcagtgtcaaaaggaggatttgaaaggga
agaaacggcaacatttgcactgctgtacaggttgagaaatatcctatttgaaagaaatagaagagtgatgg
atgtcatttctcgttcacagotttacttggatgatcttttttctgactactatgacaaacctctcagcatg
actgatatttcactcaaagaagggacccatatccgagttaacttacttaatcacaacattcccaaagggcc
ttgcatactctgtggaatggggaacttcaaaagggagacagtttatgggtgotttcagtgttctgttgatg
gtcagaagtatgtgagacttcatgcagttccttgttttgatatttggcacaagaggatgaaataa

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 51.

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 53.

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 55.

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 57.

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 59.

In some embodiments of any of the compositions described herein, the PJVK protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 61.

A non-limiting example of a human wildtype PJVK genomic DNA sequence is SEQ ID NO: 63. The exons in SEQ ID NO: 63 are: nucleotide positions 5001-5334 (exon 1); nucleotide positions 6953-7185 (exon 2) and nucleotide positions 7897-8092 (exon 3); nucleotide positions 8281-9208 (exon 4); nucleotide positions 9575-9716 (exon 5); nucleotide positions 12075-12192 (exon 6); nucleotide positions 13913-14011 (exon 7); and nucleotide positions 14547-15131 (exon 8). The introns in SEQ ID NO: 63 are: nucleotide positions 1-5000 (intron 1); nucleotide positions 5335-6952 (intron 2); nucleotide positions 7186-7896 (intron 3); nucleotide positions 8093-8280 (intron 4); nucleotide positions 9209-9574 (intron 5); nucleotide positions 9717-12074 (intron 6); nucleotide positions 12193-13912 (intron 7); and nucleotide positions 14012-14546 (intron 8).

| Human PJVK Gene Sequence (NCBI Reference Sequence: NG_012186.1) | |
|---|---|
| (SEQ ID NO: 63) | |
| 1 | tttcttgccg ggagagaggg attagaggga ttgtcoggcg tgggtctagg gtcgttgagg |
| 61 | cctaaagtta agcctttgtg gggaggacgg attgggggct gggtcacttt aagaaaaaga |
| 121 | ttctaaaatt acgaaggcag aattccaggg agtctccojg gcctggtcct gaagcttgag |
| 181 | accccggata tatacgctcc agggggctgc ggtgcgctct tcgggtcccc gagccctgtg |
| 241 | tttaggaaca cgoggggacg tccaaacacc cgctoctotc ccgccgggcg ggctcctttg |
| 301 | tcttctgggc tttcgtcgcg agatggaacg ctgggtcagt gcatcccagc aaaacacgtt |
| 361 | aggagtaaac gaaggccttt cojgggacct gggtagtojg ttgtaatggc gtttgcctgg |
| 421 | gatgcagcac ctttggtggg ccattagatg acgtgtcgct tctccagggg cttgctaggc |
| 481 | actagcagaa atgctcccct gtgcttactc ggtgttatgg aagattgctt gcacaatgct |
| 541 | attcgtacga aaggatatta ttaaaagtga tttatgggcg ggcgcggtgg ctcacgcctg |
| 601 | taatcccagc actttaggag gccgaggctg gcggatcgcc tgaagtaagc agttcgagac |
| 661 | cagcctggcc aacatggtga aaccctgtct ctactaaaaa tacaaaaatt agccgggcgt |
| 721 | ggtggcatgc gcctgtaatc ccagctactc gggaggctaa agcaggagaa tcgcttgaac |
| 781 | cojggaggca aabittgcag tgagccgaga tcgcgccact gcactccagc ctgggcgaca |
| 841 | gagtcagact ccctctcaaa aaaaaaaaaa aaaaaaaaag cgatttgttg ttttgttagc |
| 901 | atgtaagccc tggatgcttg aaacaagtga aagatgttca gaatctaatc aaattatatc |
| 961 | atctgatgct ttaattagag cctattctct cttcctctcc gtctatatat gtatgtatat |
| 1021 | agattactat ccatcactgt tttaatggat atcttattgg aagggctaca tagtcagtat |
| 1081 | ttcacaatat atgaactccg cataacacct atggcaaaac atttcagcac tcaggtaaaa |
| 1141 | ttcatttttt gccacaaaat tacttaaaaa caggccagaa ctttatatag ctcttgttaa |
| 1201 | aggttaagta gcbjggataa taatgaaaca aaaagatgca gcaggactcc atttcaaaga |
| 1261 | gagagagtct gtgtgcccaa cttcagagta agaaaccact aggtaatagt ttatatagtt |
| 1321 | acttatatgt aatgtggctt tattttttaa ataaagattt gcatataatt gtttatatct |
| 1381 | gtgaatttat atttaatatt aaacacagtt ttgtaagcat ataagatgga aaatgaatga |
| 1441 | aatgctcaat caatgagctg tatgccatga ggattcaaaa cagcactttg gtgttgccaa |
| 1501 | aattccggtg gtggacttgt gaaggaattc cctttggact aggtccactg tagattgtat |
| 1561 | cojggactat cgttagtgcc taaattgcat tgcttcccac agcactatat gcttccctta |
| 1621 | aaattggccc attgccagtg gcaaatcact atctactgtg atccttatca tagacattgg |
| 1681 | tcatattttt acgttattag ttttctgtgt atattttagt gctatagcct gccattccaa |
| 1741 | gttaaacatg ctcttagcat atcattttac accgttgatc tataattcat tacctgagca |
| 1801 | catgagcaga ggcagggaat tatacagtgg taaactactt ggtgaaacca tgctttgtat |
| 1861 | ttttaagatg tataattata tttaaaaaca agcaatgctt aattttgtgc ctgattttcc |
| 1921 | tctttaaaaa tggatttatc tgggggttgc agttgatgac gttttgattt taatatgttt |
| 1981 | gctgctgcta ccaagagctt tgtcaagcaa gttggagatg gagggagatt agttcctgtt |
| 2041 | ccaagcctca gtgaagctga caaatatcaa cctctaagtc tggtggtaaa aaagaagcga |
| 2101 | tgctttctgt ttcctagata taaatttact tcaacacctt ttacactgaa agatattctc |
| 2161 | ctaggagaca gagaaatttc agctggtaag tttaaatgtt tgggagtgcc aactcattca |
| 2221 | tctgtattat tggcaaccta aacataggtc atatatgcta cttatgttag taatacacat |
| 2281 | tttcaatgat taaagcaact tttattatga bittagtgta ataacattat taagattaac |
| 2341 | aatgccaaaa gtttgtataa tatcaaatct cttattggtc aaataatttt caaatctatc |
| 2401 | aattatttta taaattgttt taaccaaatc aacttagaat atgacatatt tcttcatcta |
| 2461 | taaaatggga tattatttgg gcttacctat gttacatggt caaatgagat gaaaataatt |
| 2521 | tgtatttatt ttttacaatt caattattac aattggtaaa ctgaaatgta ttgaagggta |
| 2581 | ggttatataa taagctgata ttttgatgag ttactactta taaacgctta ttttttcttt |
| 2641 | taattgcccg gtggatcttg atgcttaaga agttctgttg gctgaataga aaatttgagt |
| 2701 | tccttttaag aattctggct atgtgtttct ttttgttgta agattaattg aattactact |
| 2761 | ttaagtcttg gtgagtcatg ttgcctttct ctaacatttg ggtatttgag tcttcaattg |
| 2821 | ttaatttaac atgttactat atataatcta cataagataa agatgtttaa aaaatactga |
| 2881 | gtttcttctt ataaaggtat ttcatcttat caattactga attatgaaga tgaatcagat |
| 2941 | gtttcactct atggaaggcg aggtaaccat attgtaaatg acgttgggat taacgttgct |
| 3001 | ggatcagatt ccattgcagt gaaagcttca tttggtatag taaccaaaca tgaagtggaa |
| 3061 | gtatcaacat tactcaaaga aattactaca cggtcagtat aataatccta atatatttca |
| 3121 | gtgttttcat taaatacttt aggtatataa acttcattac aaagaatgct taaaacattg |
| 3181 | aggttgtatt ttactagtgt aatttgctag atatgtttga aatacattgg tagtatatcc |
| 3241 | aaactttgaa taaggataga tatcaaaaat cacttggctg aagtcagaga cctcagggcc |
| 3301 | ccagatcaag gagctaactg atgaagaggc agagaggctg cagctagaga ttgaccagaa |
| 3361 | ataggatgca gagaatcatg aggcccagat caagaatggc agccttgact ccccagggaa |
| 3421 | gcagaagact gaggaagatg aggaggagga agatgagaag gacaaaggaa aactgaagcc |
| 3481 | caacctaggc aacagggcag acctgcctga ttaccgctgg accaagaccc tgtcggagct |
| 3541 | ggacctggcg gtccctttct gtgtggactt ccagctgaaa gggaaggacg tggtggtggc |
| 3601 | atccaacggc ggcacctcca ggtggggctc aaggggcagc cagcgatcat cgatggggag |
| 3661 | ctctacaacg aagtgaaggc ggaggagagc tcgtggctca ttgaggacgg caaggtggtg |
| 3721 | actgtgcatc tggagaagat caagaagatg gagtggtgga gccgcttggt gtccagtgac |
| 3781 | cctgagatca ataccaagaa gattaaccct gagaattcca agctgtcaga cctggacagt |
| 3841 | gagactcaca tcatggtgga aaagacaatc tatgaccagt aacagaagtc catggggctg |
| 3901 | ccaacctcag acgaacagaa gaaacaggag attctgaaga agttcatgga tcagcatccg |
| 3961 | gagatggatt tttccaaggc taaattcaac tagcccctgt ttttccctcc ctgaactctt |
| 4021 | ggggctgagc tgcaaccacc caactttctt tcccactctt ctctgggact tgtgggcctc |
| 4081 | agggcttggg gcaggcatgg gactggccca ggcacacagg tcccagggca tcaggagaaa |
| 4141 | ggctggggct tggggtcttg tcctccccag ttggcctact gttacacatt aaaatgattt |
| 4201 | gcccagctaa aaaaaaaaaa aaaccacttg gcttaaacaa aacttttaat tgttccacaa |
| 4261 | aattactttg gctctacaca catttgctta attattagat ttaaataata cagcaatctg |
| 4321 | tctgcaacaa atagagtccc cttcaacaga ctttcaacag acagagtgaa acattctgca |
| 4381 | taagcatgtt ttaagaaaat agagaggaga gacaaattat tacatttctt ttgggtgtat |
| 4441 | tttctgacta ttaggattgc cttgatttac tattaggtga actatgaatg aataacacat |
| 4501 | gatagcaaaa aatgtatttg attatgtgta attagatgtt atagagtctt gtgaatgtat |
| 4561 | cttctttatt ttagaaaaat taactttgac cacagcttga tacgtcagtc aaggagcagc |
| 4621 | agaaaggcag tattgtgtgt ggtcatggag agcatccgaa ccacacgaca gtgctcactg |
| 4681 | tctgtgcatg ctggaattcg aggggaagca atgcgggtaa accacacttg ttgggttctc |
| 4741 | ttactaacta aagtgttgcc atgggaattt ttttaagtat tgaatttcta gtcaggcttg |
| 4801 | tgtattttgt gaaatgttct gatgaaagag ctgcagttgt ataggatttt gcacagcaat |
| 4861 | gtctgttcta gaatatgaaa aaaaaaaaca aatcttggga ttctaaacat gtgtaatata |
| 4921 | atgcaaaaac agtaatcctt gtgaaattac gtgcatactg gagtccctca agttactatt |
| 4981 | taagtgttgt tttaagaata aaaattgagt gagttgaatt gattttattt ttaagtgata |
| 5041 | cagtttttga ctgggcacag tggctcacgc ctgtaatcct agcattttgg gaggccgagg |
| 5101 | tgggcggatc acttgcagtc aggagttgga gatcaagttg ggcaacacag agaaacccca |
| 5161 | tctctactga aaatacaaaa attagccagg tgtggtggta cacccctgta atcccaagcc |
| 5221 | actcaggaag ctgaggcaca agaatcactt gaacctggga ggtggaggtt gcagtgagcc |
| 5281 | gagattgtgc cattgcactc taacctgggt ggcagagtga gactccgtct caaaaaaaca |
| 5341 | aaacaaaaca aaaaccccaa ccaaaaaaaa aatgtgtaac ataatgcaaa aacagtaatc |
| 5401 | cttgtgaaat tacatgcata ctggagtccc tcatattact atttaagtgc tgttttaaga |
| 5461 | ataaaaattg agtgcttaag ttgaattgat ttaagtgata gagtttttac actttgaatt |
| 5521 | ttttaaagct gaatttatag attttttttt tgagacggag tctcgctctg tccccgaggc |
| 5581 | tggagtgcag tggcgcgatc ttggctcact gcaaactccg cctccojggt tcatgccatt |
| 5641 | cttctgcctc agcctcccga gtagcbjgga ctacaggcac ctgccaccac gcccggcagt |
| 5701 | gttatccagg atggtttcga tctcctgacc tcgtgatccg ctagcctojg cctcccaaaa |
| 5761 | tgctgggatt acaggtgtga gccaccgcgc ctggcctgaa tttatatttt tatattcaaa |
| 5821 | tagtttttca aagttaatat tatcaatggc catacatgct ttattcaagg ttagttaaat |
| 5881 | cctttaaaaa atattagcgg caaggtgcag tggatcatgc ctgtaatccc agtgcttagg |
| 5941 | aagactgaga taggaagaca gcttgggacc aggagttcaa gaccaacttg agcaaaataa |
| 6001 | tgagaccttg tctctacaaa aaataaaaga attagctggt tgtggtggca catgcctata |
| 6061 | gtcccagcta ctcggaaggc tgaggcaggg ggctgcctgc ggggtttggg tcattgtctt |
| 6121 | ttagaatttg ggaggctttg aaaagccgtg cttttgtgct ggggaagatg tagcagtctc |
| 6181 | ttctccaaac cacccatttg ccttcggact tctctggggc cagcagccat ccaactaggg |
| 6241 | gccojgggcc acgggctcag ctgacgacca tgggcttcgc gtccaaccag cagtttgcag |
| 6301 | gttcgagctc aggacttggc ggggacacct taggatgcag gagtggccag gagttcaagg |
| 6361 | ttacagtagg ctatgattgt gccactgctc tccagcctgg gtgacagaga gagaccctgt |
| 6421 | ctctgaaaaa aaaattggaa aagtatctgt gtttaatttt cagctcaatt atgatattgt |
| 6481 | ttatctttgt cattcagtga cacctaataa tgacttgatt ggattaaaac caattgttag |
| 6541 | aaagataggg ttcaaggata atcttttcaa gtctttgcaa ataaaagtta ttttcgcata |
| 6601 | ttcagctgcc tgtcaaattc tgattaacat tggatcattt ttgaggcaca aactgaaaca |
| 6661 | tttacttgat tgttagcgat tgctttagaa agctactaaa agctctggag ggctttggaa |
| 6721 | tggtagacag gctaggctga aaactttaac tctggcactg ctttctaaga tttcctgtta |
| 6781 | aaaaaaaaaa aaaggcttct gctccagagc attatggttg tggtagcact aatcatgggc |
| 6841 | tgtaatacca aagtaagtga aaagtgcaga ggtatttcct aatatagacc aggagtttct |
| 6901 | gttggaccaa ttggatctct gctataaaaa atggtttaat tcaagcagaa taatccttta |
| 6961 | cttgggaatt ttttgttttg tttttggtag gattatagga aatgtttcat ttctccaaaa |
| 7021 | agctatcctt acatgttatg atccttaatt atgttattta tttattcaat atagtttcac |
| 7081 | tttatggatg aacagaatcc caagggaagg gacaaagcta ttgttttocc agcacataca |
| 7141 | accatagctt tcagtgtttt tgaactcttc atatacctgg atggtgcctt tggtgagtta |
| 7201 | agggtctgca tgaaatacaa atgattatat ttttaaggag cattcattag ggcatgtgct |
| 7261 | ctctctctct cttttctctc atttctcgtg tctaacaatt atagaagatg ctctcagcac |
| 7321 | tgtcaatctt atgctttggc aaaatagctg agaaagaccc tccgttcgct gcttccccat |
| 7381 | tccacttttc tgcttcttgg tgaaggttgt aaactctcca ctaccccatt taaaattata |
| 7441 | aataagagct aaaatgccat ctactgacaa agtattcctt agcttacatg gggagataat |
| 7501 | aaatgacctt cttataggta gtttttgatg gtggcaaaca gaggagtagg tagatagtag |
| 7561 | gatgcctgaa gtgttaggat gcccaaacca gggcctctaa agacagcttt tcttacttca |
| 7621 | gttttactta tcatagaccc agcttcccct tgcactcttc ctaaaaaata actttaaaaa |
| 7681 | gtgaaaatac ttcctccaaa attgtcacct tgtataatac ctaagtgcag ctcactttag |
| 7741 | gtctctacat ttcacaggtc tttaaggtct gttggccaac tggaacaagt ccaggtgtag |
| 7801 | cccaagggat caggaaaggc agcccctagc catggtaatt tacaaatgtt tttagacctg |
| 7861 | ttcttgttca tttctgcaat tttgtaggag gagggaatag ggagtccaga aacaaaacaa |
| 7921 | aactacatgt taagtaaatt gtctttttaa aaaatatttt taatttttgt ggctatatag |
| 7981 | tagattatat atatatttat gtatgggata catgaggtat tttgatacgg gcatacagtg |
| 8041 | cataataatc acatcatgga aaatggggta tccatcccct caagcattta tcctttgtgt |
| 8101 | tacaaacaat ccaattatac tcttttagtt atttaaaaat gtacaaattc tcagtctaag |
| 8161 | atggagaaaa aactgtacaa ttacattatt ttgactgtag tcaccctatt atgcttccaa |
| 8221 | atactaggtc ttattaagta aattctaatt ggataacttt aggtttttac agttttattg |
| 8281 | atggttggga ggaatgtaaa tggactgata tatccacaag aatttaaaat atatatataa |
| 8341 | tttggacttc cacttctagg aatttatcct acagatatat ttacacatat atgaaatgat |
| 8401 | atgagtgcaa gtatattcag tcctcaataa caagagattg gaaacaacat taaggagact |
| 8461 | aaataatggt gcatccttgc aagagaatac tatgaagcca ataagaatat aatactttta |
| 8521 | atagatattt ttcaagagct gagtataata tgtaactatt tgggcataaa taattttata |
| 8581 | cacatacatg tatatatgca tctttcttta tgcttataaa atatatggag tatttctgga |
| 8641 | agcaaacata agaaacttaa accacgggtg cccctaagcc cctgaggtag aagactgtgg |
| 8701 | ctggatacag ggtgggccag agacctattt gtcactgtat ctcctttctg tatgaatttt |
| 8761 | gtaccatgtg aatgttacct tttccaaaag tatgtaaaag tcaactttta aaaacaatac |
| 8821 | aatgaatgat ttgtactaga attcatcacc ccatcaaaca ataaaatatg aattattcaa |
| 8881 | gttataacat cttctaacaa ttttttttgt agacctttgt gtcacttcag tgtcaaaagg |
| 8941 | aggatttgaa agggaagaaa cggcaacatt tgcactgctg tacaggttga gaaatatcct |
| 9001 | atttgaaaga agtatgttta ttgaagagta ctgtgaatgt cttttttttt tctttcctgg |
| 9061 | cttaacacat gaggttttct attcagtaat ctcatctgtt aaggaaaaat ttaaatttaa |
| 9121 | aagcctggct ggtatggtgg ctcatgccta taatcccagc actttgggag gccagggcag |
| 9181 | gcagattgct agcactcaga agtttgaaac acttgggcaa tgtgagaaaa ccccatctgt |
| 9241 | acaaagaata caaaagaaaa aaaattagtt ggttgcagtg gtgtgcacct gtagtcccag |
| 9301 | ctacttgaga ggctgaggta ggaggatcac ttgagcgtgg gaggcggagg ttgtagtaag |
| 9361 | tggagattgt gccactgccc tccagottgg gtgacagagc cagaccctgt ctcaaaaaaa |
| 9421 | aaaaaaaaaa aaaaaaagcc aaattatttc atgactagtg gattcacata tttattaatg |
| 9481 | ctgtttgcat tatgtatttt tcattttcac ttctaacaca tttcttttct gtttttgtcc |
| 9541 | ttttagatag aagagtgatg gatgtcattt ctcgttcaca gctttacttg gatgatcttt |
| 9601 | tttctgacta ctatgacaaa cctctcagca tgactgatat ttcactcaaa gaagggaccc |
| 9661 | atatccgagt taacttactt aatcacaaca ttcccaaagg gccttgcata ctctgtggaa |
| 9721 | tggggaactt caaaagggag acagtttatg ggtgctttca gtgttctgtt gatggtcaga |
| 9781 | agtatgtgag acttcatgca gttccttgtt ttgatatttg gcacaagagg atgaaataaa |
| 9841 | atgaaaaatg aatacaccgt gttggtgttt taggtgcagt tgtgccacaa accttcccta |
| 9901 | aattatctag gtttgctttg atgaattaaa ttaaaatgag aaaagcaaaa agaaattaac |
| 9961 | ctgtctgggt atcatattcc ctatctataa agtagcaatt ataacagtag tgtctatttc |
| 10021 | ttaagttgtc atgaaaatta ataacattgt ttatatcaaa aaagatttac atataaaatt |
| 10081 | cagagattat gaatcatgcc agtcacttta gaatcatttt tgggatgtag t |

### Heat Shock Protein Family a (Hsp70) Member 1A (HSPA1A)

The HSPA1A gene encodes “heat shock protein family A member 1A” (HSPA1A). The human HSPA1A gene is located on chromosome 6p21.33. It contains 1 exon, encompassing ˜2400 bp (NCBI Accession No. NC_000006.12). HSPA1A encodes a 70 kDa heat shock protein, which stabilizes proteins against protein aggregation and is involved in protein folding.

Exemplary wildtype HSPA1A protein sequences are or include SEQ ID NO: 64, SEQ ID NO: 66, SEQ ID NO: 68, SEQ ID NO: 69, SEQ ID NO: 70, and SEQ ID NO: 93. Exemplary DNA sequences that encode a NDP protein are shown below.

Human Mature HSPA1A Protein (NCBI Accession No. NP_005336.3)
(SEQ ID NO: 64)
MAKAAAIGIDLGTTYSCVGVFQHGKVEIIANDQGNRTTPSYVAFTDTERLIGDAAKNQVALNPQNTVFDAK
RLIGRKFGDPVVQSDMKHWPFQVINDGDKPKVQVSYKGETKAFYPEEISSMVLTKMKEIAEAYLGYPVTNA
VITVPAYFNDSQRQATKDAGVIAGLNVLRIINEPTAAAIAYGLDRTGKGERNVLIFDLGGGTFDVSILTID
DGIFEVKATAGDTHLGGEDFDNRLVNHFVEEFKRKHKKDISQNKRAVRRLRTACERAKRTLSSSTQASLEI
DSLFEGIDFYTSITRARFEELCSDLFRSTLEPVEKALRDAKLDKAQIHDLVLVGGSTRIPKVQKLLQDFFN
GRDLNKSINPDEAVAYGAAVQAAILMGDKSENVQDLLLLDVAPLSLGLETAGGVMTALIKRNSTIPTKQTQ
IFTTYSDNQPGVLIQVYEGERAMTKDNNLLGRFELSGIPPAPRGVPQIEVTFDIDANGILNVTATDKSTGK
ANKITITNDKGRLSKEEIERMVQEAEKYKAEDEVQRERVSAKNALESYAFNMKSAVEDEGLKGKISEADKK
KVLDKCQEVISWLDANTLAEKDEFEHKRKELEQVCNPIISGLYQGAGGPGPGGFGAQGPKGGSGSGPTIEE
VD
Human Mature HSPA1A cDNA
(SEQ ID NO: 65)
atggccaaagccgcggcgatcggcatcgacctgggcaccacctactcctgcgtgggggtgttccaacacgg
caaggtggagatcatcgccaacgaccagggcaaccgcaccaccoccagctacgtggccttcacggacaccg
agoggctcatcggggatgcggccaagaaccaggtggcgctgaacccgcagaacaccgtgtttgacgcgaag
cggctgatcggccgcaagttcggcgaccoggtggtgcagtoggacatgaagcactggcctttccaggtgat
caacgacggagacaagcccaaggtgcaggtgagctacaagggggagaccaaggcattctaccccgaggaga
tctcgtccatggtgctgaccaagatgaaggagatcgccgaggcgtacctgggctaccoggtgaccaacgcg
gtgatcaccgtgccggcctacttcaacgactcgcagcgccaggccaccaaggatgogggtgtgatcgcggg
gctcaacgtgctgoggatcatcaacgagcccacggccgccgccatcgcctacggcctggacagaacgggca
agggggagcgcaacgtgctcatctttgacctgggcgggggcaccttcgacgtgtccatcctgacgatcgac
gacggcatcttcgaggtgaaggccacggccggggacacccacctgggtggggaggactttgacaacaggct
ggtgaaccacttcgtggaggagttcaagagaaaacacaagaaggacatcagccagaacaagcgagccgtga
ggcggctgcgcaccgcctgcgagagggccaagaggaccctgtcgtccagcacccaggccagcctggagatc
gactocctgtttgagggcatcgacttctacacgtccatcaccagggcgaggttcgaggagctgtgctccga
cctgttccgaagcaccctggagcccgtggagaaggctctgcgcgacgccaagctggacaaggcccagattc
acgacctggtoctggtogggggctccacccgcatccccaaggtgcagaagctgctgcaggacttcttcaac
gggcgcgacctgaacaagagcatcaaccccgacgaggctgtggcctacggggcggcggtgcaggcggccat
cctgatgggggacaagtccgagaacgtgcaggacctgctgctgctggacgtggctoccctgtcgctggggc
tggagacggccggaggcgtgatgactgccctgatcaagcgcaactccaccatccccaccaagcagacgcag
atcttcaccacctactccgacaaccaaccoggggtgctgatccaggtgtacgagggcgagagggccatgac
gaaagacaacaatctgttggggcgcttcgagctgagoggcatccctccggcccccaggggcgtgccccaga
tcgaggtgaccttcgacatcgatgccaacggcatcctgaacgtcacggccacggacaagagcaccggcaag
gccaacaagatcaccatcaccaacgacaagggccgcctgagcaaggaggagatcgagcgcatggtgcagga
ggcggagaagtacaaagoggaggacgaggtgcagcgcgagagggtgtcagccaagaacgccctggagtcct
acgccttcaacatgaagagcgccgtggaggatgaggggctcaagggcaagatcagcgaggcggacaagaag
aaggttctggacaagtgtcaagaggtcatctcgtggctggacgccaacaccttggccgagaaggacgagtt
tgagcacaagaggaaggagctggagcaggtgtgtaaccccatcatcagoggactgtaccagggtgccggtg
gtccogggcctgggggcttcggggctcagggtcccaagggagggtctgggtcaggccccaccattgaggag
gtggattag
Human Mature HSPA1A cDNA
(SEQ ID NO: 94)
atgtaccggatgcagctgctgagctgtatcgccctgtctctggccctggtcaccaattctgccaaagccgc
ggcgatcggcatcgacctgggcaccacctactcctgcgtgggggtgttccaacacggcaaggtggagatca
tcgccaacgaccagggcaaccgcaccaccoccagctacgtggccttcacggacaccgagcggctcatcggg
gatgoggccaagaaccaggtggcgctgaacccgcagaacaccgtgtttgacgcgaagoggctgatcggccg
caagttcggcgacccggtggtgcagtcggacatgaagcactggcctttccaggtgatcaacgacggagaca
agcccaaggtgcaggtgagctacaagggggagaccaaggcattctaccccgaggagatctcgtccatggtg
ctgaccaagatgaaggagatcgccgaggcgtacctgggctacccggtgaccaacgcggtgatcaccgtgcc
ggcctacttcaacgactcgcagcgccaggccaccaaggatgcgggtgtgatcgcggggctcaacgtgctgc
ggatcatcaacgagcccacggccgccgccatcgcctacggcctggacagaacgggcaagggggagcgcaac
gtgctcatctttgacctgggcgggggcaccttcgacgtgtccatcctgacgatcgacgacggcatcttcga
ggtgaaggccacggccggggacacccacctgggtggggaggactttgacaacaggctggtgaaccacttcg
tggaggagttcaagagaaaacacaagaaggacatcagccagaacaagcgagccgtgaggcggctgcgcacc
gcctgcgagagggccaagaggaccctgtcgtccagcacccaggccagcctggagatcgactccctgtttga
gggcatcgacttctacacgtccatcaccagggcgaggttcgaggagctgtgctccgacctgttccgaagca
ccctggagcccgtggagaaggctctgcgcgacgccaagctggacaaggcccagattcacgacctggtcctg
gtcgggggctccacccgcatccccaaggtgcagaagctgctgcaggacttcttcaacgggcgcgacctgaa
caagagcatcaaccccgacgaggctgtggcctacggggcggcggtgcaggcggccatcctgatgggggaca
agtccgagaacgtgcaggacctgctgctgctggacgtggctcccctgtcgctggggctggagacggccgga
ggcgtgatgactgccctgatcaagcgcaactccaccatccccaccaagcagacgcagatcttcaccaccta
ctccgacaaccaacccggggtgctgatccaggtgtacgagggcgagagggccatgacgaaagacaacaatc
tgttggggcgcttcgagctgagcggcatccctccggcccccaggggcgtgccccagatcgaggtgaccttc
gacatcgatgccaacggcatcctgaacgtcacggccacggacaagagcaccggcaaggccaacaagatcac
catcaccaacgacaagggccgcctgagcaaggaggagatcgagcgcatggtgcaggaggcggagaagtaca
aagcggaggacgaggtgcagcgcgagagggtgtcagccaagaacgccctggagtcctacgccttcaacatg
aagagcgccgtggaggatgaggggctcaagggcaagatcagcgaggcggacaagaagaaggttctggacaa
gtgtcaagaggtcatctcgtggctggacgccaacaccttggccgagaaggacgagtttgagcacaagagga
aggagctggagcaggtgtgtaaccccatcatcagcggactgtaccagggtgccggtggtcccgggcctggg
ggcttcggggctcagggtcccaagggagggtctgggtcaggccccaccattgaggaggtggattaa

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 64.

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 66.

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 68.

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 69.

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 70.

In some embodiments of any of the compositions described herein, the HSPA1A protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 94.

Mouse Mature HSPA1A Protein (NCBI Accession No. NP_034609.2)
(SEQ ID NO: 66)
MAKNTAIGIDLGTTYSCVGVFQHGKVEIIANDQGNRTTPSYVAFTDTERLIGDAAKNQVALNPQNTVFDAK
RLIGRKFGDAVVQSDMKHWPFQVVNDGDKPKVQVNYKGESRSFFPEEISSMVLTKMKEIAEAYLGHPVTNA
VITVPAYFNDSQRQATKDAGVIAGLNVLRIINEPTAAAIAYGLDRTGKGERNVLIFDLGGGTFDVSILTID
DGIFEVKATAGDTHLGGEDFDNRLVSHFVEEFKRKHKKDISQNKRAVRRLRTACERAKRTLSSSTQASLEI
DSLFEGIDFYTSITRARFEELCSDLFRGTLEPVEKALRDAKMDKAQIHDLVLVGGSTRIPKVQKLLQDFFN
GRDLNKSINPDEAVAYGAAVQAAILMGDKSENVQDLLLLDVAPLSLGLETAGGVMTALIKRNSTIPTKQTQ
TFTTYSDNQPGVLIQVYEGERAMTRDNNLLGRFELSGIPPAPRGVPQIEVTFDIDANGILNVTATDKSTGK
ANKITITNDKGRLSKEEIERMVQEAERYKAEDEVQRDRVAAKNALESYAFNMKSAVEDEGLKGKLSEADKK
KVLDKCQEVISWLDSNTLADKEEFVHKREELERVCSPIISGLYQGAGAPGAGGFGAQAPKGASGSGPTIEE
VD
Mouse Mature HSPA1A cDNA
(SEQ ID NO: 67)
atggccaag aacacggcga tcggcatcga cctgggcacc acctactcgt gcgtgggcgt
gttccagcac ggcaaggtgg agatcatcgc caacgaccag ggcaaccgca cgacccccag
ctacgtggcc ttcaccgaca ccgagcgcct catcggagac gccgccaaga accaggtggc
gctgaacccg cagaacaccg tgttcgacgc gaagcggctg atcggccgca agttcggcga
tgcggtggtg cagtccgaca tgaagcactg gccottccag gtggtgaacg acggcgacaa
goccaaggtg caggtgaact acaagggcga gagccggtog ttottccogg aggagatctc
gtccatggtg ctgacgaaga tgaaggagat cgctgaggcg tacctgggcc acccggtgac
caacgcggtg atcacggtgc ccgcctactt caacgactct cagojgcagg ccaccaagga
cgcgggcgtg atcgccggtc taaacgtgct gcggatcatc aacgagccca cggcggccgc
catcgcctac gggctggacc ggaccggcaa gggcgagcgc aacgtgctca tcttcgacct
ggggggcggc acgttcgacg tgtccatcct gacgatcgac gacggcatct tcgaggtgaa
ggccacggcg ggcgacacgc acctgggagg ggaggacttc gacaaccggc tggtgagcca
cttcgtggag gagttcaaga ggaagcacaa gaaggacatc agccagaaca agcgcgcggt
gojgcggctg cgcactgcgt gtgagagggc caagaggacg ctgtcgtcca gcacccaggc
cagcctggag atcgactctc tgttcgaggg catcgacttc tacacatcca tcacgcgggc
gog-gttcga gagctgtgct cagacctgtt ccgcggcacg ctggagcccg tggagaaggc
cctgcgcgac gccaagatgg acaaggcgca gatccacgac ctggtgctgg tgggcggctc
gacgcgcatc cccaaggtgc agaagctgct gcaggacttc ttcaacgggc gcgacctgaa
caagagcatc aaccoggacg aggcggtggc ctacggggcg goggtgcagg cggccatcct
gatgggggac aagtojgaga acgtgcagga cctgctgctg ctggacgtgg cgccgctgtc
gctgggcctg gagactgcgg gojgcgtgat gacggcgctc atcaagcgca actccaccat
ccccaccaag cagacgcaga ccttcaccac ctactcggac aaccagcccg gggtgctgat
ccaggtgtac gagggcgaga gggccatgac gcgcgacaac aacctgctgg ggcgcttcga
actgagojgc atcccgccgg cgcccagggg cgtgccacag atcgaggtga ccttcgacat
cgacgccaac ggcatcctga acgtcacggc caccgacaag agcaccggca aggccaacaa
gatcaccatc accaacgaca agggccgcct gagcaaggag gagatcgagc gcatggtgca
ggaggccgag cgctacaagg ccgaggacga ggtgcagcgc gacagggtgg ccgccaagaa
cgcgctcgaa tcctatgcct tcaacatgaa gagcgccgtg gaggacgagg gtctcaaggg
caagctcagc gaggctgaca agaagaaggt gctggacaag tgccaggagg tcatctcctg
gctggactcc aacacgctgg ccgacaagga ggagttcgtg cacaagojgg aggagctgga
gojggtgtgc agccccatca tcagtgggct gtaccagggt gojggtgctc ctggggctgg
gggcttoggg gcccaggcgc ccaagggagc ctctggctca ggacccacca tcgaggaggt
ggattaga
Rhesus Monkey Mature HSPA1A Protein (NCBI Accession No. XP_014991489.2)
(SEQ ID NO: 68)
MAKAAAIGIDLGTTYSCVGVFQHGKVEIIANDQGNRTTPSYVAFTDTERLIGDAAKNQVALNPQNTVFDAK
RLIGRKFGDPVVQSDMKHWPFQVINDGDKPKVQVSYKGETKAFYPEEISSMVLTKMKEIAEAYLGYPVTNA
VITVPAYFNDSQRQATKDAGVIAGLNVLRIINEPTAAAIAYGLDRTGKGERNVLIFDLGGGTEDVSILTID
DGIFEVKATAGDTHLGGEDFDNRLVNHFVEEFKRKHKKDISQNKRAVRRLRTACERAKRTLSSSTQASLEI
DSLFEGIDFYTSITRARFEELCSDLFRSTLEPVEKALRDAKLDKAQIHDLVLVGGSTRIPKVQKLLQDFFN
GRDLNKSINPDEAVAYGAAVQAAILMGDKSENVQDLLLLDVAPLSLGLETAGGVMTALIKRNSTIPTKQTQ
IFTTYSDNQPGVLIQVYEGERAMTKDNNLLGRFELSGIPPAPRGVPQIEVTFDIDANGILNVTATDKSTGK
ANKITITNDKGRLSKEEIERMVQEAEKYKAEDEVQRERVSAKNALESYAFNMKSAVEDEGLKGKISEADKK
KVLDKCQEVISWLDANTLAEKDEFEHKRKELEQVCNPIISGLYQGAGGPGPGGFGAQGPKGGSGSGPTIEE
VD
Rat Mature HSPA1A Protein (NCBI Accession No. NP_114177.2)
(SEQ ID NO: 69)
MAKKTAIGIDLGTTYSCVGVFQHGKVEIIANDQGNRTTPSYVAFTDTERLIGDAAKNQVALNPQNTVFDAK
RLIGRKFGDPVVQSDMKHWPFQVVNDGDKPKVQVNYKGENRSFYPEEISSMVLTKMKEIAEAYLGHPVTNA
VITVPAYFNDSQRQATKDAGVIAGLNVLRIINEPTAAAIAYGLDRTGKGERNVLIFDLGGGTFDVSILTID
DGIFEVKATAGDTHLGGEDFDNRLVSHFVEEFKRKHKKDISQNKRAVRRLRTACERAKRTLSSSTQASLEI
DSLFEGIDFYTSITRARFEELCSDLFRGTLEPVEKALRDAKLDKAQIHDLVLVGGSTRIPKVQKLLQDFFN
GRDLNKSINPDEAVAYGAAVQAAILMGDKSENVQDLLLLDVAPLSLGLETAGGVMTALIKRNSTIPTKQTQ
TFTTYSDNQPGVLIQVYEGERAMTRDNNLLGRFELSGIPPAPRGVPQIEVTFDIDANGILNVTATDKSTGK
ANKITITNDKGRLSKEEIERMVQEAERYKAEDEVQRERVAAKNALESYAFNMKSAVEDEGLKGKISEADKK
KVLDKCQEVISWLDSNTLAEKEEFVHKREELERVCNPIISGLYQGAGAPGAGGFGAQAPKGGSGSGPTIEE
VD
Cattle HSPA1A Protein (NCBI Accession No. NP_976067.3)
(SEQ ID NO: 70)
MAKNMAIGIDLGTTYSCVGVFQHGKVEIIANDQGNRTTPSYVAFTDTERLIGDAAKNQVALNPQNTVFDAK
RLIGRKFGDPVVQSDMKHWPFRVINDGDKPKVQVSYKGETKAFYPEEISSMVLTKMKEIAEAYLGHPVTNA
VITVPAYFNDSQRQATKDAGVIAGLNVLRIINEPTAAAIAYGLDRTGKGERNVLIFDLGGGTFDVSILTID
DGIFEVKATAGDTHLGGEDFDNRLVNHFVEEFKRKHKKDISQNKRAVRRLRTACERAKRTLSSSTQASLEI
DSLFEGIDFYTSITRARFEELCSDLFRSTLEPVEKALRDAKLDKAQIHDLVLVGGSTRIPKVQKLLQDFFN
GRDLNKSINPDEAVAYGAAVQAAILMGDKSENVQDLLLLDVAPLSLGLETAGGVMTALIKRNSTIPTKQTQ
IFTTYSDNQPGVLIQVYEGERAMTRDNNLLGRFELSGIPPAPRGVPQIEVTFDIDANGILNVTATDKSTGK
ANKITITNDKGRLSKEEIERMVQEAEKYKAEDEVQRERVSAKNALESYAFNMKSAVEDEGLKGKISEADKK
KVLDKCQEVISWLDANTLAEKDEFEHKRKELEQVCNPIISRLYQGAGGPGAGGFGAQGPKGGSGSGPTIEE
VD
Human Mature HSPA1A Protein
(SEQ ID NO: 93)
AKAAAIGIDLGTTYSCVGVFQHGKVEIIANDQGNRTTPSYVAFTDTERLIGDAAKNQVALNPQNTVFDAKR
LIGRKFGDPVVQSDMKHWPFQVINDGDKPKVQVSYKGETKAFYPEEISSMVLTKMKEIAEAYLGYPVTNAV
ITVPAYFNDSQRQATKDAGVIAGLNVLRIINEPTAAAIAYGLDRTGKGERNVLIFDLGGGTFDVSILTIDD
GIFEVKATAGDTHLGGEDFDNRLVNHFVEEFKRKHKKDISQNKRAVRRLRTACERAKRTLSSSTQASLEID
SLFEGIDFYTSITRARFEELCSDLFRSTLEPVEKALRDAKLDKAQIHDLVLVGGSTRIPKVQKLLQDFFNG
RDLNKSINPDEAVAYGAAVQAAILMGDKSENVQDLLLLDVAPLSLGLETAGGVMTALIKRNSTIPTKQTQI
FTTYSDNQPGVLIQVYEGERAMTKDNNLLGRFELSGIPPAPRGVPQIEVTFDIDANGILNVTATDKSTGKA
NKITITNDKGRLSKEEIERMVQEAEKYKAEDEVQRERVSAKNALESYAFNMKSAVEDEGLKGKISEADKKK
VLDKCQEVISWLDANTLAEKDEFEHKRKELEQVCNPIISGLYQGAGGPGPGGFGAQGPKGGSGSGPTIEEV
D

A non-limiting example of a human wildtype NDP genomic DNA sequence is SEQ ID NO: 71.

| Human HSPA1A Gene Sequence(NCBI Reference Sequence NC_00000612) | |
|---|---|
| (SEQ ID NO: 71) | |
| 1 aacggctagc ctgaggagct gctgcgacag tccactacct ttttcgagag tgactcccgt | 61 |
| tgtcccaagg cttcccagag cgaacctgtg cggctgcagg caccggcgcg tcgagtttcc | 121 |
| ggcgtccgga aggaccgagc tcttctcgcg gatccagtgt tccgtttcca gcccccaatc | 181 |
| tcagagcgga gccgacagag agcagggaac cggcatggcc aaagccgcgg cgatcggcat | 241 |
| cgacctgggc accacctact cctgoitggg ggtgttccaa cacggcaagg tggagatcat | 301 |
| cgccaacgac cagggcaacc gcaccacccc cagctacgtg gccttcacgg acaccgagcg | 361 |
| gctcatcggg gatgojgcca agaaccaggt ggcgctgaac ccgcagaaca ccgtgtttga | 421 |
| cgcgaagcgg ctgattggcc gcaagttcgg cgaccoggtg gtgcagtojg acatgaagca | 481 |
| ctggcctttc caggtgatca acgacggaga caagcccaag gtgcaggtga gctacaaggg | 541 |
| ggagaccaag gcattctacc ccgaggagat ctcgtccatg gtgctgacca agatgaagga | 601 |
| gatcgccgag gcgtacctgg gctaccoggt gaccaacgcg gtgatcaccg tgccggccta | 661 |
| cttcaacgac tcgcagcgcc aggccaccaa ggatgojggt gtgatcgcgg ggctcaacgt | 721 |
| gctgcggatc atcaacgagc ccacggccgc cgccatcgcc tacggcctgg acagaacggg | 781 |
| caagggggag cgcaacgtgc tcatctttga cctgggcggg ggcaccttcg acgtgtccat | 841 |
| cctgacgatc gacgacggca tcttcgaggt gaaggccacg gccggggaca cccacctggg | 901 |
| tggggaggac tttgacaaca ggctggtgaa ccacttcgtg gaggagttca agagaaaaca | 961 |
| caagaaggac atcagccaga acaagcgagc cgtgaggcgg ctgcgcaccg cctgcgagag | 1021 |
| ggccaagagg accctgtcgt ccagcaccca ggccagcctg gagatcgact ccctgtttga | 1081 |
| gggcatcgac ttctacacgt ccatcaccag ggcgaggttc gaggagctgt gctccgacct | 1141 |
| gttccgaagc accctggagc ccgtggagaa ggctctgcgc gacgccaagc tggacaaggc | 1201 |
| ccagattcac gacctggtcc tggtoggggg ctccacccgc atccccaagg tgcagaagct | 1261 |
| gctgcaggac ttcttcaacg ggcgcgacct gaacaagagc atcaaccccg acgaggctgt | 1321 |
| ggcctacggg gojgcggtgc aggcggccat cctgatgggg gacaagtccg agaacgtgca | 1381 |
| ggacctgctg ctgctggacg tggctcccct gtcgcbgggg ctggagacgg ccggaggcgt | 1441 |
| gatgactgcc ctgatcaagc gcaactccac catccccacc aagcagacgc agatcttcac | 1501 |
| cacctactcc gacaaccaac cojgggtgct gatccaggtg tacgagggcg agagggccat | 1561 |
| gacgaaagac aacaatctgt tggggcgctt cgagctgagc ggcatccctc cggcccccag | 1621 |
| gggcgtgccc cagatcgagg tgaccttcga catcgatgcc aacggcatcc tgaacgtcac | 1681 |
| ggccacggac aagagcaccg gcaaggccaa caagatcacc atcaccaacg acaagggccg | 1741 |
| cctgagcaag gaggagatcg agcgcatggt gcaggaggcg gagaagtaca aagcggagga | 1801 |
| cgaggtgcag cgcgagaggg tgtcagccaa gaacgccctg gagtcctacg ccttcaacat | 1861 |
| gaagagcgcc gtggaggatg aggggctcaa gggcaagatc agcgaggcgg acaagaagaa | 1921 |
| ggtgctggac aagtgtcaag aggtcatctc gtggctggac gccaacacct tggccgagaa | 1981 |
| ggacgagttt gagcacaaga ggaaggagct ggagcaggtg tgtaacccca tcatcagcgg | 2041 |
| actgtaccag ggtgccggtg gtcccgggcc tgggggcttc ggggctcagg gtcccaaggg | 2101 |
| agggtctggg tcaggcccca ccattgagga ggtagattag gggcctttcc aagattgctg | 2161 |
| tttttgtttt ggagcttcaa gactttgcat ttcctagtat ttctgtttgt cagttctcaa | 2221 |
| tttcctgtgt ttgcaatgtt gaaatttttt ggtgaagtac tgaacttgct ttttttccgg | 2281 |
| tttctacatg cagagatgaa tttatactgc catcttacga ctatttcttc tttttaatac | 2341 |
| acttaactca ggccattttt taagttggtt acttcaaagt aaataaactt taaaattcaa | |

Human HSPA1A Gene Sequence
(SEQ ID NO: 94)
atgtaccggatgcagctgctgagctgtatcgccctgtctctggccctggtcaccaattctgccaaagccgc
ggcgatcggcatcgacctgggcaccacctactcctgcgtgggggtgttccaacacggcaaggtggagatca
tcgccaacgaccagggcaaccgcaccacccccagctacgtggccttcacggacaccgagcggctcatcggg
gatgcggccaagaaccaggtggcgctgaacccgcagaacaccgtgtttgacgcgaagcggctgatcggccg
caagttcggcgacccggtggtgcagtcggacatgaagcactggcctttccaggtgatcaacgacggagaca
agcccaaggtgcaggtgagctacaagggggagaccaaggcattctaccccgaggagatctcgtccatggtg
ctgaccaagatgaaggagatcgccgaggcgtacctgggctacccggtgaccaacgcggtgatcaccgtgcc
ggcctacttcaacgactcgcagcgccaggccaccaaggatgcgggtgtgatcgcggggctcaacgtgctgc
ggatcatcaacgagcccacggccgccgccatcgcctacggcctggacagaacgggcaagggggagcgcaac
gtgctcatctttgacctgggcgggggcaccttcgacgtgtccatcctgacgatcgacgacggcatcttcga
ggtgaaggccacggccggggacacccacctgggtggggaggactttgacaacaggctggtgaaccacttcg
tggaggagttcaagagaaaacacaagaaggacatcagccagaacaagcgagccgtgaggcggctgcgcacc
gcctgcgagagggccaagaggaccctgtcgtccagcacccaggccagcctggagatcgactccctgtttga
gggcatcgacttctacacgtccatcaccagggcgaggttcgaggagctgtgctccgacctgttccgaagca
ccctggagcccgtggagaaggctctgcgcgacgccaagctggacaaggcccagattcacgacctggtcctg
gtcgggggctccacccgcatccccaaggtgcagaagctgctgcaggacttcttcaacgggcgcgacctgaa
caagagcatcaaccccgacgaggctgtggcctacggggcggcggtgcaggcggccatcctgatgggggaca
agtccgagaacgtgcaggacctgctgctgctggacgtggctcccctgtcgctggggctggagacggccgga
ggcgtgatgactgccctgatcaagcgcaactccaccatccccaccaagcagacgcagatcttcaccaccta
ctccgacaaccaacccggggtgctgatccaggtgtacgagggcgagagggccatgacgaaagacaacaatc
tgttggggcgcttcgagctgagcggcatccctccggcccccaggggcgtgccccagatcgaggtgaccttc
gacatcgatgccaacggcatcctgaacgtcacggccacggacaagagcaccggcaaggccaacaagatcac
catcaccaacgacaagggccgcctgagcaaggaggagatcgagcgcatggtgcaggaggcggagaagtaca
aagcggaggacgaggtgcagcgcgagagggtgtcagccaagaacgccctggagtcctacgccttcaacatg
aagagcgccgtggaggatgaggggctcaagggcaagatcagcgaggcggacaagaagaaggttctggacaa
gtgtcaagaggtcatctcgtggctggacgccaacaccttggccgagaaggacgagtttgagcacaagagga
aggagctggagcaggtgtgtaaccccatcatcagcggactgtaccagggtgccggtggtcccgggcctggg
ggcttcggggctcagggtcccaagggagggtctgggtcaggccccaccattgaggaggtggattaa

### Vascular Endothelial Growth Factor (VEGF)

The VEGF gene encodes vascular endothelial growth factor (VEGF), formerly known as fms-like tyrosine kinase (Flt-1). The VEGF protein is a heparin-biding protein that induces migration and proliferation of vascular endothelial cells.

The human VEGF gene is located on chromosome 6p21. It contains 8 exons encompassing ˜16 kilobases (kb) (NCBI Accession No. NG_008732.1). The full-length wildtype VEGF protein expressed from the human VEGF gene is 412 amino acids in length.

An exemplary human wildtype VEGF protein is or includes the sequence of SEQ ID NO: 72, SEQ ID NO: 74, SEQ ID NO: 76, SEQ ID NO: 77, SEQ ID NO: 78, or SEQ ID NO: 79. Non-limiting examples of nucleic acid encoding a wildtype VEGF protein are or include SEQ ID NO: 73 and SEQ ID NO: 75.

Non-limiting examples of protein and nucleotide sequences encoding a wildtype VEGF protein are shown below.

Human VEGF Transcript Variant 1 Protein Sequence
(SEQ ID NO: 72)
MTDRQTDTAPSPSYHLLPGRRRTVDAAASRGQGPEPAPGGGVEGVGARGVALKLFVQLLGCSRFGGAVVRA
GEAEPSGAARSASSGREEPQPEEGEEEEEKEEERGPQWRLGARKPGSWTGEAAVCADSAPAARAPQALARA
SGRGGRVARRGAEESGPPHSPSRRGSASRAGPGRASETMNFLLSWVHWSLALLLYLHHAKWSQAAPMAEGG
GQNHHEVVKFMDVYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKKSVRGKGKGQKRKRKKSRYKSWSVYVGARCCLMP
WSLPGPHPCGPCSERRKHLFVQDPQTCKCSCKNTDSRCKARQLELNERTCRCDKPRR
Human VEGF Transcript Variant 1 cDNA
(SEQ ID NO: 73)
ctgacqqacagacagacagacaccgccoccagocccagctaccacctcctcccoggccggcggcggacagt
ggacgcggcggcgagccgogggcaggggccggagoccgcgccoggaggcggggtggagggggtcggggctc
goggcgtcgcactgaaacttttcgtccaacttctgggctgttctcgcttoggaggagccgtggtccgcgcg
ggggaagccgagccgagoggagccgcgagaagtqctagctogggccgggaggagccgcagccggaggaggg
ggaggaggaagaagagaaggaagaggagagggggccgcagtggcgactoggcgctoggaagccgggctcat
ggacgggtgaggcggcggtgtgcgcagacagtgctccagccgcgcgcgctccccaggccctggcccgggcc
tcgggccggggaggaagagtagctcgccgaggcgccgaggagagcgggccgccccacagcccgagccggag
agggagcgcgagccgcgccggccccggtcgggcctccgaaaccatgaactttctgctgtcttgggtgcatt
ggagccttgccttgctgctctacctccaccatgccaagtggtcccaggctgcacccatggcagaaggagga
gggcagaatcatcacgaagtggtgaagttcatggatgtctatcagcgcagctactgccatccaatcgagac
cctggtggacatcttccaggagtaccctgatgagatcgagtacatcttcaagccatcctgtgtgcccctga
tgcgatgcgggggctgctgcaatgacgagggcctggagtgtgtgcccactgaggagtccaacatcaccatg
cagattatgcggatcaaacctcaccaaggccagcacataggagagatgagcttcctacagcacaacaaatg
tgaatgcagaccaaagaaagatagagcaagacaagaaaaaaaatcagttcgaggaaagggaaaggggcaaa
aacgaaagcgcaagaaatcccggtataagtcctggagcgtgtacgttggtgcccgctgctgtctaatgccc
tggagcctccctggcccccatccctgtgggccttgctcagagcggagaaagcatttgtttgtacaagatcc
gcagacgtgtaaatgttcctgcaaaaacacagactcgcgttgcaaggcgaggcagcttgagttaaacgaac
gtacttgcagatgtgacaagccgaggcggtga
Human VEGF Transcript Variant 3 Protein Sequence
(SEQ ID NO: 74)
MTDRQTDTAPSPSYHLLPGRRRTVDAAASRGQGPEPAPGGGVEGVGARGVALKLFVQLLGCSRFGGAVVRA
GEAEPSGAARSASSGREEPQPEEGEEEEEKEEERGPQWRLGARKPGSWTGEAAVCADSAPAARAPQALARA
SGRGGRVARRGAEESGPPHSPSRRGSASRAGPGRASETMNFLLSWVHWSLALLLYLHHAKWSQAAPMAEGG
GQNHHEVVKFMDVYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKKSVRGKGKGQKRKRKKSRPCGPCSERRKHLFVQD
PQTCKCSCKNTDSRCKARQLELNERTCRCDKPRR
Human VEGF Transcript Variant 3 cDNA
(SEQ ID NO: 75)
ctgacggacagacagacagacaccgccoccagccccagctaccacctcctcccoggccggcggcggacagt
ggacgcggcggcgagccgcgggcaggggccggagcccgcgccoggaggcggggtggagggggtcggggctc
goggcgtcgcactgaaacttttcgtccaacttctgggctgttctcgcttcggaggagccgtggtccgcgcg
ggggaagccgagccgagoggagccgcgagaagtgctagctogggccgggaggagccgcagccggaggaggg
ggaggaggaagaagagaaggaagaggagagggggccgcagtggcgactoggcgctoggaagccgggctcat
ggacgggtgaggcggcggtgtgcgcagacagtgctccagccgcgcgcgctccccaggccctggcccgggcc
togggccggggaggaagagtagctcgccgaggcgccgaggagagogggccgccccacagcccgagccggag
agggagcgcgagccgcgccggcccoggtogggcctccgaaaccatgaactttctgctgtcttgggtgcatt
ggagccttgccttgctgctctacctccaccatgccaagtggtoccaggctgcacccatggcagaaggagga
gggcagaatcatcacgaagtggtgaagttcatggatgtctatcagcgcagctactgccatccaatcgagac
cctggtggacatcttccaggagtaccctgatgagatcgagtacatcttcaagccatcctgtgtgcccctga
tgcgatgogggggctgctgcaatgacgagggcctggagtgtgtgcccactgaggagtccaacatcaccatg
cagattatgoggatcaaacctcaccaaggccagcacataggagagatgagottcctacagcacaacaaatg
tgaatgcagaccaaagaaagatagagcaagacaagaaaaaaaatcagttcgaggaaagggaaaggggcaaa
aacgaaagcgcaagaaatcccgtocctgtgggccttgctcagagoggagaaagcatttgtttgtacaagat
ccgcagacgtgtaaatgttcctgcaaaaacacagactcgcgttgcaaggcgaggcagcttgagttaa
acgaacgtacttgcagatgtgacaagccgaggcggtga
Mature Human VEGF-A
(SEQ ID NO: 76)
APMAEGGGQNHHEVVKFMDVYQRSYCHPIETLVDIFQEYPDEIEYIFKPSCVPLMRCGGCCNDEGLECVPT
EESNITMQIMRIKPHQGQHIGEMSFLQHNKCECRPKKDRARQEKKSVRGKGKGQKRKRKKSRYKSWSVYVG
ARCCLMPWSLPGPHPCGPCSERRKHLFVQDPQTCKCSCKNTDSRCKARQLELNERTCRCDKPRR
Mature Human VEGF-B
(SEQ ID NO: 77)
PVSQPDAPGHQRKVVSWIDVYTRATCQPREVVVPLTVELMGTVAKQLVPSCVTVQRCGGCCPDDGLECVPT
GQHQVRMQILMIRYPSSQLGEMSLEEHSQCECRPKKKDSAVKPDRAATPHHRPQPRSVPGWDSAPGAPSPA
DITHPTPAPGPSAHAAPSTTSALTPGPAAAAADAAASSVAKGGA
Mature Human VEGF-C
(SEQ ID NO: 78)
AHYNTEILKSIDNEWRKTQCMPREVCIDVGKEFGVATNTFFKPPCVSVYRCGGCCNSEGLQCMNTSTSYLS
KTLFEITVPLSQGPKPVTISFANHTSCRCMSKLDVYRQVHSIIRR
Mature Human VEGF-D
(SEQ ID NO: 79)
FAATFYDIETLKVIDEEWQRTQCSPRETCVEVASELGKSTNTFFKPPCVNVFRCGGCCNEESLICMNTSTS
YISKQLFEISVPLTSVPELVPVKVANHTGCKCLPTAPRHPYSIIRR

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 72.

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 74.

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 76.

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 77.

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 78.

In some embodiments of any of the compositions described herein, the VEGF protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 79.

A non-limiting example of a human wildtype VEGF genomic DNA sequence is SEQ ID NO: 80. The exons in SEQ ID NO: 80 are: nucleotide positions 1-1104 (exon 1); nucleotide positions 4133-4184 (exon 2) and nucleotide positions 7261-7457 (exon 3); nucleotide positions 8252-8328 (exon 4); nucleotide positions 8681-8710 (exon 5); nucleotide positions 10524-10646 (exon 6); nucleotide positions 11748-11879 (exon 7); and nucleotide positions 14333-16279 (exon 8). The introns in SEQ ID NO: 80 are: nucleotide positions 1105-4132 (intron 1); nucleotide positions 4185-7260 (intron 2); nucleotide positions 7458-8251 (intron 3); nucleotide positions 8329-8680 (intron 4); nucleotide positions 8711-10523 (intron 5); nucleotide positions 10647-11747 (intron 6); and nucleotide positions 11880-14332 (intron 7).

| Human VEGF-A Gene (NCBI Accession No.NG_008732.1) | |
|---|---|
| (SEQ ID NO: 80) | |
| 1 | cgcctgtaat cccagcactc tgggaggcag aggtgggccg atcacttgag gtcaggagtt |
| 61 | cgagaccagc ctgggcaaca tggtgaaaca ccatctctac taaaaacaca aaaattagcc |
| 121 | aggtgtggtg gcaggcacct gcagtcccag ctactccgga ggctgaggca ggagaattgc |
| 181 | tcgaacctgg gaggcagggg ttgcagtgag ccgacatggc gccactgcac tccagtctgg |
| 241 | gcgacagagt gagaccctat ctcaaaaaaa aaaaaaaaaa aaaaagaccc aactcaagta |
| 301 | tcatctccag gaagccttcc cctactccca gcaattaaat gctcctcaga gaattcccat |
| 361 | ttttggttta ctotttggtt tacctccaga caggaagocc ccactgacac tgttgtagtc |
| 421 | ccagggtgca acacaaagca gagatcacaa gctgagttta ataattgctt gtggaataca |
| 481 | tgtcccaagc cacctcctgc aggaagccct tccagatgcc cattctagcc agtctggctc |
| 541 | tttgcttcca taccttcaca acacttgtgc ctcccccagg gcctctttct catcttgctt |
| 601 | tctggggcag ctgtgtgcac atttgtctgt gtgcagcaac tctctaaggc agggattttt |
| 661 | actcctattt ttgatgaggg gagctgtggc tcagagaggt tgaataacct aaggccacac |
| 721 | agtgagtggc agagccagga atgtgacttg ggtccatttg aatccaaagt ccctgtactt |
| 781 | tccactgccc tacctagatg tccctgtacc tcctataaaa tcagcatgga gcctggtgcc |
| 841 | tggtagtccc tacaaatatt cacaaattgg agcttagctc agctctcagg caaggcccag |
| 901 | gtcaaaaggg cagatacagc tttgggacct tagttgccac cacatgccat accttcttcc |
| 961 | cagcagaagg actccctcca agacagggta ggggtggagg atgtgaacag gggcagaaat |
| 1021 | gggcatgttt tggggtcaga cttggaggaa tagcagagat tggagtgtca gaaggtgagc |
| 1081 | atgcctgggg gtgttgggga gatgcaattc atcagggaca gcttagtgtc aggggattag |
| 1141 | actggggccc atgaaggaga ggcagaggct gatgggccta ggggtggtgt gggtaggtga |
| 1201 | gcttccccag acagtgactc tgccctgccc tctctccagc taggtcctct tccccattcc |
| 1261 | ttcccccttt cctgactgga tcctcttggg agagttaccc tccttggctt cctctgctcc |
| 1321 | aatcttttta tcagttggcc atcattactt atcattacct caagtcaaac ctccagatcc |
| 1381 | acatggggct aggacattgg cactggacca aagaggccct ttcctttgct ttctctttgt |
| 1441 | ctttttaatg cttbittgca aagacctagg cggggagaga gagagagaga gagagacaga |
| 1501 | gattgaccca cagtcagggt cagggaattg aggggaacca acccaattct ctctccttca |
| 1561 | attcaccagg tttgtatcct gcccttcctg cagatcagtg tcctgctagt cacctggggg |
| 1621 | tcaggggatg gagtgaagga caagacctcc ttccattgca gtgaagccac ttggagaaat |
| 1681 | gtgtggaaaa cagcaagacc cagtgactct ctcctcacct tctttccaat ctcaggagag |
| 1741 | attttgtccc ttcatccacc ggcttctaga ttaaccaccc acacccacac aggcgagagt |
| 1801 | ttccctgaat attggaggtg acaggacatc aggacaaagt acaactattg tgccttggcc |
| 1861 | caatcactac tttctttgtc tggggccgcc gctggctcct ggctgccttt cgcacttttc |
| 1921 | tccaccccca cccctttctc cccttccccc tcacctggaa caccttcccc ttcctccttg |
| 1981 | gctccttctg aactgccttc agagccacag actgbjggga gtggccactg cgctcccaag |
| 2041 | gtgaggccct ccaagojggg ccgagtttgc ccctcaactg ggagccagca tgacctctgt |
| 2101 | gtgggctgct ctctgcttca ctgccccttc ccccaatctg ctaggtgacc ctgggcccct |
| 2161 | ttgtgccctc tctgggcctt cggaggattc tttggggaga cagtctgctc tgacgcccct |
| 2221 | tcccctgcag caagcagcct ggggagggag gtgaggataa gtgaagtcaa gttgttcagg |
| 2281 | gggctaagcc catggaaggg aagatgccac agagatacat gtggtcttgt gattgttgtt |
| 2341 | ttgtgctttt tccccttttt tgaaagctca ggtgactagg tgacttgagc ttttaatttg |
| 2401 | gtgacaatgt gggcactggc tgagtcctta agagtacatt gttgtaaatg ccggtgacaa |
| 2461 | cacactgggg catgggatcc agagttaacc cctccaggtc acagccaggt tatatctcca |
| 2521 | caatgaaggg gggaggtggg ccatactttc tcgccctaat gaaggtagct caaaaacccc |
| 2581 | taggccaggt tgtaatccta gccttatata aaaggaattc tgtgccctca ctcccctgga |
| 2641 | tccctgggca aagccccaga gggaaacaca aacaggttgt tgtaacacac cttgctgggt |
| 2701 | accaccatgg aggacagttg gcttatgggg gtggggggtg cctggggcca cggagtgact |
| 2761 | ggtgatggct atccctcctt ggaacccctc cagcctcctc ttagcttcag atttgtttat |
| 2821 | ttgtttttta ctaagacctg ctctttcagg tctgttggct cttttagggg ctgaagaagg |
| 2881 | ccgagttgag aagggatgca agggaggggg ccagaatgag cccttagggc tcagagcctc |
| 2941 | catcctgccc caagatgtct acagcttgtg ctcctggggt gctagaggcg cacaaggagg |
| 3001 | aaagttagtg gcttcccttc catatcccgt tcatcagcct agagcatgga gcccaggtga |
| 3061 | ggaggcctgc ctgggagggg gccctgagcc aggaaataaa catttactaa ctgtacaaag |
| 3121 | accttgtccc tgctgcbggg gagcctgcca agtggtggag acaggactag tgcacgaatg |
| 3181 | atggaaaggg agggttgggg tgggtgggag ccagcccttt tcctcataag ggccttagga |
| 3241 | caccataccg atggaactgg gggtactggg gaggtaacct agcacctcca ccaaaccaca |
| 3301 | gcaacatgtg ctgaggatgg ggctgactag gtaagctccc tggagcgttt tggttaaatt |
| 3361 | gagggaaatt gctgcattcc cattctcagt ccatgcctcc acagaggcta tgccagctgt |
| 3421 | aggccagacc ctggcaagat ctgggtggat aatcagactg actggtccca ctcttcccac |
| 3481 | aggcctcaga gccccaactt tgttccctgg ggcagcctgg aaatagccag gtcagaaacc |
| 3541 | agctaggaat ttttccaagc tgcttcctat atgcaagaat gggatggggc ctttgggagc |
| 3601 | acttagggaa gatgtggaga gttggaggaa aagggggctt ggaggtaagg gaggggactg |
| 3661 | ggggaaggat aggggagaag ctgtgagcct ggagaagtag ccaagggatc ctgagggaat |
| 3721 | gggggagctg agacgaaacc cccatttcta ttcagaagat gagctatgag tctgggcttg |
| 3781 | ggctgataga agccttggcc cctggcctgg tgggagctct gggcagctgg cctacagacg |
| 3841 | ttccttagtg ctggcgggta ggtttgaatc atcacgcagg ccctggcctc cacccgcccc |
| 3901 | caccagcccc ctggcctcag ttccctggca acatctgggg ttgggggggc agcaggaaca |
| 3961 | agggcctctg tctgcccagc tgcctccccc tttgggtttt gccagactcc acagtgcata |
| 4021 | cgtgggctcc aacaggtcct cttccctccc agtcactgac taaccccgga accacacagc |
| 4081 | ttcccgttct cagctccaca aacttggtgc caaattcttc tcccctggga agcatccctg |
| 4141 | gacacttccc aaaggacccc agtcactcca gcctgttggc tgccgctcac tttgatgtct |
| 4201 | gcaggccaga tgagggctcc agatggcaca ttgtcagagg gacacactgt ggcccctgtg |
| 4261 | cccagccctg ggctctctgt acatgaagca actccagtcc caaatatgta gctgtttggg |
| 4321 | aggtcagaaa tagggggtcc aggagcaaac tccccccacc ccctttccaa agcccattcc |
| 4381 | ctctttagcc agagccgggg tgtgcagacg gcagtcacta gggggcgctc ggccaccaca |
| 4441 | gggaagctgg gtgaatggag cgagcagcgt cttcgagagt gaggacgtgt gtgtctgtgt |
| 4501 | gggtgagtga gtgtgtgcgt gtggggttga gggcgttgga gcjgggagaa ggccaggggt |
| 4561 | cactccagga ttccaataga tctgtgtgtc cctctcccca cccgtccctg tccggctctc |
| 4621 | cgccttcccc tgcccccttc aatattccta gcaaagaggg aacggctctc aggccctgtc |
| 4681 | cgcacgtaac ctcactttcc tgctccctcc tcgccaatgc cccgcgggcg cgtgtctctg |
| 4741 | gacagagttt cogggggcgg atgggtaatt ttcaggctgt gaaccttggt gggggtcgag |
| 4801 | cttccccttc attgojgcgg gctgcgggcc aggcttcact gagcgtccgc agagcccggg |
| 4861 | cccgagccgc gtgtggaagg gctgaggctc gcctgtcccc gccccccggg gcgggccggg |
| 4921 | ggcggggtcc cggcggggcg gagccatgcg cccccccctt ttttttttaa aagtcggctg |
| 4981 | gtagojggga ggatcgcgga ggcttggggc agccgggtag ctcggaggtc gtggcgctgg |
| 5041 | gggctagcac cagcgctctg tcgggaggcg cagcggttag gtggaccggt cagcggactc |
| 5101 | accggccagg gcgctoggtg ctggaatttg atattcattg atccgggttt tatccctctt |
| 5161 | cttttttctt aaacattttt ttttaaaact gtattgtttc tcgttttaat ttatttttgc |
| 5221 | ttgccattcc ccacttgaat cgggccgacg gcttggggag attgctctac ttccccaaat |
| 5281 | cactgtggat tttggaaacc agcagaaaga ggaaagaggt agcaagagct ccagagagaa |
| 5341 | gtcgaggaag agagagacgg ggtcagagag agcgcgcggg cgtgcgagca gcgaaagcga |
| 5401 | caggggcaaa gtgagtgacc tgcttttggg ggtgaccgcc ggagcgcggc gtgagccctc |
| 5461 | ccccttggga tcccgcagct gaccagtcgc gctgacggac agacagacag acaccgcccc |
| 5521 | cagccccagc taccacctcc tcccojgccg gcggcggaca gtggacgcgg cggcgagccg |
| 5581 | cgggcagggg ccggagcccg cgcccggagg cggggtggag ggggtcgggg ctcgcggcgt |
| 5641 | cgcactgaaa cttttcgtcc aacttctggg ctgttctcgc ttcggaggag ccgtggtccg |
| 5701 | cgcgggggaa gccgagccga gcggagccgc gagaagtgct agctcgggcc gggaggagcc |
| 5761 | gcagccggag gagggggagg aggaagaaga gaaggaagag gagagggggc cgcagtggcg |
| 5821 | actojgcgct cggaagccgg gctcatggac gggtgaggcg gcggtgtgcg cagacagtgc |
| 5881 | tccagccgcg cgcgctcccc aggccctggc cojggcctcg ggccggggag gaagagtagc |
| 5941 | tcgccgaggc gccgaggaga gojggccgcc ccacagcccg agccggagag ggagcgcgag |
| 6001 | ccgcgccggc ccoggtojgg cctccgaaac catgaacttt ctgctgtctt gggtgcattg |
| 6061 | gagccttgcc ttgctgctct acctccacca tgccaaggta agoggtcgtg ccctgctggc |
| 6121 | gccgcgggcc gctgcgagcg cctctccogg ctggggacgt gcgtgcgagc gcgcgcgtgg |
| 6181 | gggctccgtg ccccacgcgg gtccatgggc accaggcgtg cggcgtcccc ctctgtcgtc |
| 6241 | ttaggtgcag ggggaggggg cgcgcgcgct aggtgggagg gtacccggag agaggctcac |
| 6301 | cgcccacgcg ggccctgccc acccaccgga gtcaccgcac gtacgatctg ggccgaccag |
| 6361 | ccgagggcgg gagccggagg aggaggccga gggggcbggg cttgcgttgc cgctgccggc |
| 6421 | tgaagtttgc tccojgccgc tggtccogga cgaactggaa gtctgagcag cgggggcggg |
| 6481 | agccagagac cagtgggcag ggggtgctcg gaccttggac cgcgggaggg cagagagcgt |
| 6541 | ggagggggca gggcgcagga gggagagggg gcttgctgtc actgccactc ggtctcttca |
| 6601 | gccctcgccg cgagtttggg aaaagttttg gggtggattg ctgoggggac cccccctccc |
| 6661 | tgctgggcca cctgcgccgc gccaaccccg ccoitccccg ctcgcgtccc gctcggtgcc |
| 6721 | cgccctcccc cgccojgccg ggtgcgcgcg gcgcggagcc gattacatca gcccgggcct |
| 6781 | ggccggccgc gtgttcccgg agcctcggct gcccgaatgg ggagcccaga gtggcgagcg |
| 6841 | gcacccctcc ccccgccagc cctccgcggg aaggtgacct ctcgaggtag ccccagcccg |
| 6901 | gggatccaga gaaccatccc taccccttcc tactgtctcc agaccctacc tctgcccagt |
| 6961 | gctaggagga atttcctgac gccccttctc ttcacccatt tcctttttag cctggagaga |
| 7021 | agcccctgtc accccgctta ttttcatttc tctctgcgga gaagatccat ctaacccctt |
| 7081 | tctggcccca gagtccaggg aaaggatgat cactgtcaga agtcgtggcg cgggagccca |
| 7141 | ctgggcgctt tgtcacattc caccgaaagt cccgacttgg tgacagtgtg cttcccttcc |
| 7201 | ctcgccaaca gttccgagtg agcbitgott tagctotcgt gggggtgggt caagggagga |
| 7261 | tttgaagagt cattgcccca ctttaccctt ttggagaaat ggcttgaaat ttgctgtgac |
| 7321 | acgggcagca tgggaatagt ccttcctgaa ccctggaaag gagctcctgc cagccttgca |
| 7381 | cacactttgt cctggtgaaa ggcagccctg gagcaggtgt ttttttggaa ctccaaacct |
| 7441 | gcccacccaa cttgcttctg aaagggactc taaagggtcc ctttccgctc ctctctgacg |
| 7501 | ccttccctca gccagaattc ccttggagag gaggcaagag gaaagccatg gacaggggtc |
| 7561 | gctgctaaca ccgcaagttc ctcagaccct ggcacaaagg ccttggctac aggcctccaa |
| 7621 | gtagggagga gggggaggag tggctgcctg gccacagtgt gaccttcaga ggcccccaga |
| 7681 | gaaggacacc tggcccctgc ctgcctagaa ccgcccctcc tgtgctccct ggccttggaa |
| 7741 | ggggtatgaa atttccgtcc cctttcctcc ttggggccca ggaggagtgg agggtcccgg |
| 7801 | gagaatattg tcagggggaa ggcagggggt gtcatgggaa tgggtgaggg ggctgaggtg |
| 7861 | cagaatccag ggggtccctg caggagccgc agtggtaagc tgtccagctg gaagcctggt |
| 7921 | aactgtbitt ttctcttgag aggggcttcc tgtgaccttg gctgtctctg ggagcagggc |
| 7981 | tggggtacct gagtggggtg catttggggt gtttgggaag gagagggaaa gaaagatgga |
| 8041 | cagtgggact ctcccctagc agggtctggt gttccgtagg ctagagtgcc cctctgctct |
| 8101 | gcgagtgctg ggcgggaggg gagttggtga gagctggaga cccccaggaa gggctggcag |
| 8161 | aagcctttcc ttttgggtgc bitcaggtcc gcatgtcttg gcttgttgac cttcacagct |
| 8221 | tctggcgagg ggaggaatga tctgatgcgg gtggggaggg ttagaggagg cctcaggcct |
| 8281 | aaggtggtgc agggggcccc ctaggggctg ggcagtgcca aggcataaaa gccttccctg |
| 8341 | gtccctggtg gcatttgaag gtgcccaggt gagaggggct tggcacctcc tcaccctggg |
| 8401 | agggagaaga aaccagggaa caggtaggag tgggagacag gtgaggcttt ggaaatctat |
| 8461 | tgaggctctg gagagatttg tgtagagagg aaaatgtggt tctcccccag ggtctcctcc |
| 8521 | tgggttttta ccctctaagc aacctgtggg catgcbjggt tattcctaag gactagaaga |
| 8581 | gcttggatgg gggagggtgg ttggtgccct toggtcctcg gcacccccct ccgtctccaa |
| 8641 | caccagctca ccctggtatt tttcatgtca gcaggagaag gtcaccatgt tgtttttctc |
| 8701 | gcccctagtc cttccttcct gccccagtcc aaatttgtcc tcctatttga ccttaatact |
| 8761 | taccatggct ttggaccagg gaactagggg gatagtgaga gcagggagag ggaagtgtgg |
| 8821 | ggaaggtaca ggggacctcg acagtgaagc attctggggt tttcctcctg catttcgagc |
| 8881 | tccccagccc ccaacatctg gttagtcttt aacttcctcg ggttcataac catagcagtc |
| 8941 | caggagtggt gggcatattc tttgcccgtg gggacccccg gttgtgtcct gttcgactca |
| 9001 | gaagacttgg agaagccaga ggctgttggt gggagggaag tgaggaggga ggaggggctg |
| 9061 | ggtggcbggg cctgtgcacc ccagcccctg cccatgccca tgccttgctc tctttctgtc |
| 9121 | ctcagtggtc ccaggctgca cccatggcag aaggaggagg gcagaatcat cacgaaggtg |
| 9181 | agtccccctg gcbgttggat ggggttccct gtcctctcag gggatgggtg gatggcctaa |
| 9241 | ttcctttttc ttcagaactg tggggaggaa ggggaagggg cacaggaata taaggatcaa |
| 9301 | gaaagaaaga gcbjggcacc acgaggttca ccctcagttt cgtgaggact ctccgctgtt |
| 9361 | caggtctctg ctagaagtag gacttgttgc ctttttcttc tgctctttcc agtaaaattt |
| 9421 | tatttggaga aggagtcgtg cgcacagagc aggaagacag tgttcaggga tcctaggtgt |
| 9481 | tgggggaagt gtcccttgtt tcccotagct cccaggggag agtggacatt tagtgtcatt |
| 9541 | tcctatatag acatgtccca tttgtgggaa ctgtgaccct tcctgtgtga gctggaggca |
| 9601 | cagagggctc agcctaatgg gatctctcct cccttccctg gtttgcattc ctttgggggt |
| 9661 | ggagaaaacc ccatttgact atgttcgggt gctgtgaact tccctcccag gccagcagag |
| 9721 | ggctggctgt agctcccagg cgccccgccc ccctgcccaa ccccgagtcc gcctgccttt |
| 9781 | bittccgttg tggtttggat cctcccattt ctctggggac accctggctc tccccaccac |
| 9841 | tgactgtggc ctgtgctctc cacctctggg gagggaaggc cctggggtct tccttcccgc |
| 9901 | gagtttccct gacctaaatc tggcgtggct gggtagtggc cagcagtggt gatgcccagc |
| 9961 | ctgttctgcc tcctccttcc ccaccccagg agccctttcc ttggcctagg acctggcttc |
| 10021 | tcagccactg accggccccc tgcttccagt gcgccactta ccccttccag cttcccagtg |
| 10081 | gtctctggtc tgggagaggc aggacaaagg tctttgtttg ctggagaaaa ggttgtctgc |
| 10141 | gataaataag gaaaaccacg aaagcctggt bittggagtg tacgtgtgtg ctcccccagg |
| 10201 | cagtggaggc cagccctcct tggaggggcg gctgcctgat gaaggatgcg ggtgaggttc |
| 10261 | cccgcctcca cctcccatgg gacttgggga ttcattccaa ggggaagctt tttgggggaa |
| 10321 | ttcctacccc aggtcttttt accctcagtt accaacccct tgcccaggcc agaccttcct |
| 10381 | gctatcccct cctgggccac aagcctggcc ctcctctgtc ccaattgtga tgaaggggca |
| 10441 | gttcaaaact tcttgattag tcatcttctc ccctatcgac ttggctttaa aaaatgacct |
| 10501 | tttcagactt ctagtctcgt tcactctttt tgatgatgct ttgccgtaac ccttcgtggg |
| 10561 | tagagaagga ttctgtgccc attggtggtc tggataaaag aaatagagac ctcacaggaa |
| 10621 | gcagtggact ggcctgtttc cccactgttc tttctgtttt cacacctgtg gccttctccc |
| 10681 | caccttcttc ccaatcaacc tattgtgtac atagcccccc tcattgtcct ttattcttct |
| 10741 | ggaaagcaga ccttggaggg aggagtgagg gggaggctca gcbgtggtct ctggggggtg |
| 10801 | ggggttggga gcbggggtgg aagtccacga agcatacact taagatgctt tggtgaagtt |
| 10861 | ctaaacttca tattacccag gctgaaaaaa gagcacttgt tcctagggct ggaaatggaa |
| 10921 | gccaaaacac cacctttttc agcctgtttc agcatcttta gagatcagcc caacccactt |
| 10981 | acacagttga gcagagttgg aggcctagag aggggaggga ctggcccaag gtcataccaa |
| 11041 | ctcatggcca gagcctgggc ctcctcactg gccaggtgtt atttcttccc tctgggtagg |
| 11101 | gaacctattt cagggacagg attgctatgt ggtagtggtg gtggggtgcg ataggcgtgg |
| 11161 | caggcbjggc cacaatttgg agtagtcatg ccagagtcct gcatttattt attctcaagg |
| 11221 | gccccgcctc tgtggcccag aattacccct tcatgctcca gtgcacccca ggcttcgtgg |
| 11281 | ccagcctggg aaactgtctc taccctggtc tcccttcaga tcagcttcta gaaatgtttc |
| 11341 | gtggctacag tggcagcact gttttttcca tgatgcaagc agtttgccct cttgggcggg |
| 11401 | gttatcagtg gctggcaggg ctggcacagc gtgtccgccc actgccacct gtgggttcca |
| 11461 | ggagggccca gcccctgtgc tgatgcccac caccttctca gctcatgtct ggggaagagg |
| 11521 | actggcaggg ggaaaggtgc ctcctcctga aaggtgcctc ctctgttttt gcctaatata |
| 11581 | ggcttgggaa cactttgatg tcagctaatt ctgactcctt tacttactag ctgtgcggcc |
| 11641 | ttggggcaac ttacttagcc tctttgagcc tcctgttccc catctgtaaa atggaatctc |
| 11701 | aatagtgtct aatagtacca tgtggagaaa cttgtgtgaa atgatagctg tggactactg |
| 11761 | tacacagtac tcaggatgta gtaagtgctc aataaacagc bittggtatg gttgacgtta |
| 11821 | tggtagtggt tgtggggagg acgtaggaaa ctggagacta gcttggcaaa gctggctctt |
| 11881 | cctcctttta gggaaagctt agagcatccc catggggtat acccatactc agactgtcct |
| 11941 | ctggcatcga ggttggccca ggattcagtt cagcbgtcac agtgaggtgg cgggatcaga |
| 12001 | tgtggcaggc catgtccctt ggaacttgag tacatcgtgt gatctctgga atgaaaacag |
| 12061 | gccttcacca gtgttgatgg tggaaagctt agggaagtgc ttcaaacaca gtaggaggga |
| 12121 | cttacgttag attttggaag gacttgcctg attcggaagc tccaaagagt ggcattacag |
| 12181 | agcbjggtgg agagaggggc tagccatctt ttgtgtcgcc caccgggctc atgtgtcatc |
| 12241 | gcctctcatg cagtggtgaa gttcatggat gtctatcagc gcagctactg ccatccaatc |
| 12301 | gagaccctgg tggacatctt ccaggagtac cctgatgaga tcgagtacat cttcaagcca |
| 12361 | tcctgtgtgc ccctgatgcg atgojggggc tgctgcaatg acgagggcct ggagtgtgtg |
| 12421 | cccactgagg agtccaacat caccatgcag gtgggcatct ttgggaagtg gggcaagggg |
| 12481 | gggataggga ggggggtaac actttgggaa caggtggtcc caggtcgttt cctggctaga |
| 12541 | tttgccttgt ctggctcctg cccctgagtt gcacagggga ggtatggtgg ggtcttgcct |
| 12601 | tctgtggaga agatgcttca ttcccagccc aggttcccag caagccccaa ccatctcctt |
| 12661 | ctccctgatg gttgcccatg ggctcaggag gggacagatg gatgcctgtg tcaggagccc |
| 12721 | ctctctccct ctcttggaga gagtcctgag tgccccccct tcttgggggc tttgtttggg |
| 12781 | aagctggatg agcctggtcc atggagagtt taaaaagtct tttggtgtta cctggtaatg |
| 12841 | gggcacatct cagcccagat agggtgggag ggagctgtga aacacaggga gggggttgct |
| 12901 | ttcgggtatc tactaggagt cagggtgaag cctagagagg atgaaagaag gggaggggat |
| 12961 | ggggagtggt aagaacctag gatttgaatt cccagcctgg ccaacccttg cagccatgtc |
| 13021 | ttggcctcaa gtggaacaag ggctccttga ggccagcagg gttgggggag ttggggtggg |
| 13081 | cctgagcctc tttcctgcta gagctcttgg tcctccctgc ctccaccacc catccctgct |
| 13141 | ctgcagaacc cctgggtgct gagtggcagg agccccaggg ttgtcccatc tgggtatggc |
| 13201 | tggcbgggtc actaacctct gtgatctgct tccttccttt ccagattatg cggatcaaac |
| 13261 | ctcaccaagg ccagcacata ggagagatga gcttcctaca gcacaacaaa tgtgaatgca |
| 13321 | ggtgaggatg tagtcacgga ttcattatca gcaagtggct gcagggtgcc tgatctgtgc |
| 13381 | cagggttaag catgctgtac tttttggccc ccgtccagct tcccgctatg tgacctttgg |
| 13441 | cattttactt caatgtgcct cagtttctac atctgtaaaa tgggcacaat agtagtatac |
| 13501 | ttcatagcat tgttataatg attaaacaag ttatatatga aaagattaaa acagtgttgc |
| 13561 | tccataataa atgcbgtttt tactgtgatt attattgttg ttatccctat cattatcatc |
| 13621 | accatcttaa cccttccctg ttttgctctt ttctctctcc ctacccattg cagaccaaag |
| 13681 | aaagatagag caagacaaga aaagtaagtg gccctgactt tagcacttct ccctctccat |
| 13741 | ggccggttgt cttggtttgg ggctcttggc tacctctgtt gggggctccc atagcctccc |
| 13801 | tgggtcaggg acttggtctt gtgggggact tgtggtggca gcaacaatgg gatggagcca |
| 13861 | actccaggat gatggctcta gggctagtga gaaaacatag ccaggagcct ggcacttcct |
| 13921 | ttggaaggga caatgccttc tgggtctcca gatcattcct gaccaggact tgctgtttcg |
| 13981 | gtgtgtcagg gggcactgtg gacactggct cactggcttg ctctaggaca cccacagtgg |
| 14041 | ggagagggag tgggtggcag agaggccagc ttttgtgtgt cagaggaaat ggcctctttt |
| 14101 | ggtggctgct gtgacggtgc agttggatgc gaggccggct ggagggtggt ttctcagtgc |
| 14161 | atgccctcct gtaggcggca ggcggcagac acacagccct cttggccagg gagaaaaagt |
| 14221 | tgaatgttgg tcattttcag aggcttgtga gtgctccgtg ttaaggggca ggtaggatgg |
| 14281 | ggtgggggac aaggtctggc ggcagtaacc cttcaagaca gggtgggcgg ctggcatcag |
| 14341 | caagagcttg cagggaaaga gagactgaga gagagcacct gtgccctgcc ctttccccca |
| 14401 | caccatcttg tctgcctcca gtgcbgtgcg gacattgaag cccccaccag gcctcaaccc |
| 14461 | cttgcctctt ccctcagctc ccagcttcca gagcgagggg atgcggaaac cttccttcca |
| 14521 | ccctttggtg ctttctccta agggggacag acttgccctc tctggtccct tctccccctc |
| 14581 | ctttcttccc tgtgacagac atcctgaggt gtgttctctt gggcttggca ggcatggaga |
| 14641 | gctctggttc tcttgaaggg gacaggctac agcctgcccc ccttcctgtt tccccaaatg |
| 14701 | actgctctgc catggggaga gtagggggct cgcctgggct cggaagagtg tctggtgaga |
| 14761 | tggtgtagca ggctttgaca ggcbjgggag agaactccct gccaagtacc gcccaagcct |
| 14821 | ctcctcccca gacctcctta actcccaccc catcctgctg cctgcccagg gctccaggac |
| 14881 | acccagccct gcctcccagt ccaggtcgtg ctgagcaggc tggtgttgct cttggttccg |
| 14941 | tgccagctcc caaggtagcc gcttccccca caccgggatt cccagaggtt ctgtcgcagt |
| 15001 | tgcaaatgaa ggcacaaggc ctgatacaca gccctccctc ccactcctgc tccccatcca |
| 15061 | ggcaggtctc tgaccttctc cccaaagtct ggcctacctt ttatcacccc cggaccttca |
| 15121 | gggtcagact tggacagggc tgctgggcaa agagccttcc ctcaggcttt gccccctgcc |
| 15181 | ggggactggg agccactgtg agtgtggaga cctttgggtc ctgtgccctc cacccagtct |
| 15241 | cggcttccca ccaaagcctt gtcaggggct gggtttgcca tcccatggtg ggcagcgtga |
| 15301 | ggagaagaaa gagccatcga gtgcttgctg cccagacacg cctgtgtgcg cccgcgcatg |
| 15361 | cctccccaga gaccacctgc ctcctgacac ttcctccggg aagojgccct gtgtggcttt |
| 15421 | gctttggtog ttcccccatc cctgcccacc ttaccacttc ttttactccc cccaccgccc |
| 15481 | ccgctctctc tctgtctctg tttttttatt ttccagaaaa tcagttcgag gaaagggaaa |
| 15541 | ggggcaaaaa cgaaagcgca agaaatcccg gtataagtcc tggagcgtgt acgttggtgc |
| 15601 | ccgctgctgt ctaatgccct ggagcctccc tggcccccag tacaacctcc gcctgccatt |
| 15661 | ccctgtaacc ctgcctccct cccctggtcc ttccctggct ctcatcctcc tggcccgtgt |
| 15721 | ctctctctca ctctctcact ccactaattg gcaccaacgg gtagatttgg tggtggcatt |
| 15781 | gctggtccag ggttggggtg aatgggggtg ccgacttggc ctggaggatt aagggagggg |
| 15841 | accctggctt ggcbjggcac cgattttctc tcacccactg ggcactggtg gcgggcccat |
| 15901 | gttggcacag gtgcctgctc acccaactgg tttccattgc tctaggcttc tgcactcgtc |
| 15961 | tggaagctga gggtggtggg gagggcagac atggcccaag aagggcbgtg aatgactgga |
| 16021 | ggcagcttgc tgaatgactc cttggctgaa ggaggagctt gggtgggatc agacaccatg |
| 16081 | tggcggcctc ccttcatctg gtggaagtgc cctggctcct cacggaggtg gggcctctgg |
| 16141 | aggggagccc cctattccgg cccaacccat ggcacccaca gaggcctcct tgcagggcag |
| 16201 | cctcttcctc tgggtoggag gcbgtggtgg gccctgccct gggccctctg gccaccagcg |
| 16261 | gcctggcctg gggacaccgc ctccgggctt agcctcccat cacaccctac tttagcccac |
| 16321 | cttggtggaa gggcctggac atgagccttg cacggggaga aggtggcccc tgattgccat |
| 16381 | ccccagcagg tgaagagtca aggcgtgctc cgatgggggc aacagcagtt gggtccctgt |
| 16441 | ggcctgagac tcacccttgt ctcccagaga cacagcattg ccccttatgg cagcctctcc |
| 16501 | ctgcactctc tgcccgtctg tgcccgcctc ttcctgojgc aggtgtccta gccagtgctg |
| 16561 | cctctttccg ccgctctctc tgtcttttgc tgtagcgctc ggatccttcc agggcctggg |
| 16621 | ggctgaccgg ctgggtgggg gtgcagctgc ggacatgtta gggggtgttg catggtgatt |
| 16681 | ttttttctct ctctctgctg atgctctagc ttagatgtct ttccttttgc ctttttgcag |
| 16741 | tccctgtggg ccttgctcag agcggagaaa gcatttgttt gtacaagatc cgcagacgtg |
| 16801 | taaatgttcc tgcaaaaaca cagactcgcg ttgcaaggcg aggcagcttg agttaaacga |
| 16861 | acgtacttgc aggttggttc ccagagggca agcaagtcag agaggggcat cacacagaga |
| 16921 | tggggagaga gagagagaaa gagagtgagc gagcgagcga gojggagagc gcctgagagg |
| 16981 | ggccagctgc ttgctcagtt tctagctgcc tgcctggtga ctgctgcctt ctctgctttt |
| 17041 | aaggcccctg tggtgggctg caggcactgg tccagcctgg cggggcctgt tccgaggttg |
| 17101 | ccctggttgc ctgagtggta ggcbggtgtg gcttagtgta gtggtgtgga cgcaagctgt |
| 17161 | gtgttgtgtc ctgtggtcct tctgctcata gtggctgttg gtcctgatgt tattactacc |
| 17221 | tctggtagta atgctgagaa gctgaaagcc gattccaggt gtggacaatg tcaacaaagc |
| 17281 | acagatgctc tcgctggggc cttgcctcgg ccctttgaag tctgcatggc tgggcttctc |
| 17341 | actcactcag tgtttcttgc tgggggaagg aattgagtct cccacttcag actgggcctc |
| 17401 | cctgaggaaa gggttgtgtc tccccactca gactgaggtt ccctgagggt agggctgtgt |
| 17461 | ctctcccctc cgacctgggc tccctgatag ggctgtctcc ccgctcagac tgaggctccc |
| 17521 | tcaggccagg gctatgtctc cctcctcaga ctggggctct gagggcaagg ggtctggctg |
| 17581 | ttcgtttagg atggggcact tttgcctaca cactgaagga gctgtagcat ccaagaatac |
| 17641 | tagatacctt taatcctcca ccagtcatgg tgacaacccc aagcagccca cacattttca |
| 17701 | agtgccccca ggatgcgtgg agggaggggt ctgtgcccat tctcctgaca ttagcctgtg |
| 17761 | agctccgtaa gccojggcct cgtttacgta cctttgtgag cccojggcat ctgtacctct |
| 17821 | ttcctttgcc catactgggg accaaggaag tgtcaagtgc atgagtgaat gtgtgactca |
| 17881 | gttcagaggg tgaggtcagg agcacagggt cgggacaggt ggctggcatc ttttaatgcc |
| 17941 | ttagcttatg ttctttatac caacttggcc tgtgctcaga gtgagggagg ccctgggggt |
| 18001 | cagggtaagc gtcagtcagg gaggcaagac tttgtgggga tttcctagac agggccaagg |
| 18061 | cacccccagc tcaccccgag gcbgtgttag ggaagtcctt ggagtgtctc ccctccccca |
| 18121 | gcaatgttct tgtggcttgt gtgtgctcag gggatgctgg gaaccaggcc tgggtagttg |
| 18181 | gtgtggggtg ctgtctgtct tggccctatg tgaaaccaag agggcgtata ttagtgctgg |
| 18241 | ggtgggggct ctgcctaact tcagggctgg atgaggggag tctcagttcc ccaggggtcc |
| 18301 | ttgggaaaga taagggactt gacattttag ggtttttagg tgattattct gctgatgggg |
| 18361 | gtttgtgtga agtgacctgg gagctaactg aagttactct aacctcccaa tacctttacc |
| 18421 | caacccccaa gctggctgta tctgggaata tcagtttcca aaattggagg cttaggactc |
| 18481 | cgtttogggg ctccccagaa gggtagggcc tgttctgcct ccttctcaca atcacccagg |
| 18541 | ggcaggggca tgctgagaaa gttcttggag gccccctttg cttcagctgg agtagtgaag |
| 18601 | ccgccgaatt gtotctoccc atcctaagtg aagcagcata tttgaaagga aagacaacct |
| 18661 | gttacctggg cctgcaacct ccaggcagct caagagagat gaggcctaca gccacagtgg |
| 18721 | gaggggacat ggggaatgga gatggtccct caccttcctg gggcctcctg ctctacgcta |
| 18781 | ccccctojgg agcctcctgt ccccagggca ggcccttgcc attgttggtc acccggccaa |
| 18841 | gcctctctgc ctcaggcgtt ctcccagaag atctgcccac tctcttcccc acaccagccc |
| 18901 | ctagagactg aactgaaaac cctcctcagc agggagcctc ttctgattaa cttcatccag |
| 18961 | ctctggtcac ccatcagctc ttaaaatgtc aagtggggac tgttctttgg tatccgttca |
| 19021 | tttgttgctt tgtaaagtgt tcccatgtcc ttgtcttgtc tcaagtagat tgcaagctca |
| 19081 | ggagggtaga ctgggagccc ctgagtggag ctgctgctca ggccggggct ccctgagggc |
| 19141 | agggctgggg ctgttctcat actggggctt tctgccccag gaccacacct tcctgtcctc |
| 19201 | tctgctctta tggtgccgga ggctgcagtg acccaggggc ccccaggaat ggggaggccg |
| 19261 | cctgcctcat cgccaggcct cctcacttgg ccctaacccc agcctttgtt ttccatttcc |
| 19321 | ctcagatgtg acaagccgag goggtgagcc gggcaggagg aaggagcctc cctcagggtt |
| 19381 | tojggaacca gatctctcac caggaaagac tgatacagaa cgatcgatac agaaaccacg |
| 19441 | ctgccgccac cacaccatca ccatcgacag aacagtcctt aatccagaaa cctgaaatga |
| 19501 | aggaagagga gactctgcgc agagcacttt gggtccggag ggcgagactc cggcggaagc |
| 19561 | attccojggc gggtgaccca gcacggtccc tcttggaatt ggattcgcca ttttattttt |
| 19621 | cttgctgcta aatcaccgag cccggaagat tagagagttt tatttctggg attcctgtag |
| 19681 | acacacccac ccacatacat acatttatat atatatatat tatatatata taaaaataaa |
| 19741 | tatctctatt ttatatatat aaaatatata tattcttttt ttaaattaac agtgctaatg |
| 19801 | ttattggtgt cttcactgga tgtatttgac tgcbgtggac ttgagttggg aggggaatgt |
| 19861 | tcccactcag atcctgacag ggaagaggag gagatgagag actctggcat gatctttttt |
| 19921 | ttgtcccact tggtggggcc agggtcctct cccctgccca ggaatgtgca aggccagggc |
| 19981 | atgggggcaa atatgaccca gttttgggaa caccgacaaa cccagccctg gcgctgagcc |
| 20041 | tctctacccc aggtcagacg gacagaaaga cagatcacag gtacagggat gaggacaccg |
| 20101 | gctctgacca ggagtttggg gagcttcagg acattgctgt gctttgggga ttccctccac |
| 20161 | atgctgcacg cgcatctcgc ccccaggggc actgcctgga agattcagga gcctgggcgg |
| 20221 | ccttcgctta ctctcacctg cttctgagtt gcccaggaga ccactggcag atgtcccggc |
| 20281 | gaagagaaga gacacattgt tggaagaagc agcccatgac agctcccctt cctgggactc |
| 20341 | gccctcatcc tcttcctgct ccccttcctg gggtgcagcc taaaaggacc tatgtcctca |
| 20401 | caccattgaa accactagtt ctgtcccccc aggagacctg gttgtgtgtg tgtgagtggt |
| 20461 | tgaccttcct ccatcccctg gtccttccct tcccttcccg aggcacagag agacagggca |
| 20521 | ggatccacgt gcccattgtg gaggcagaga aaagagaaag tgttttatat acggtactta |
| 20581 | tttaatatcc ctttttaatt agaaattaaa acagttaatt taattaaaga gtagggtttt |
| 20641 | ttttcagtat tcttggttaa tatttaattt caactattta tgagatgtat cttttgctct |
| 20701 | ctcttgctct cttatttgta coggtttttg tatataaaat tcatgtttcc aatctctctc |
| 20761 | tccctgatcg gtgacagtca ctagcttatc ttgaacagat atttaatttt gctaacactc |
| 20821 | agctctgccc tccccgatcc cctggctccc cagcacacat tcctttgaaa taaggtttca |
| 20881 | atatacatct acatactata tatatatttg gcaacttgta tttgbitgta tatatatata |
| 20941 | tatatgttta tgtatatatg tgattctgat aaaatagaca ttgctattct gttttttata |
| 21001 | tgtaaaaaca aaacaagaaa aaatagagaa ttctacatac taaatctctc tcctttttta |
| 21061 | attttaatat ttgttatcat ttatttattg gtgctactgt ttatccgtaa taattgtggg |
| 21121 | gaaaagatat taacatcacg tctttgtctc tagtgcagtt tttcgagata ttccgtagta |
| 21181 | catatttatt tttaaacaac gacaaagaaa tacagatata tcttaaaaaa aaaaaagcat |
| 21241 | tttgtattaa agaatttaat tctgatctca aagctcctct tggtttctcc ttctccattg |
| 21301 | aatccttgct ctagacttcc tcccgccccc tttccctctt cctctgggga acatggcatt |
| 21361 | tgtcttgggt ctgggaaagg tacgtctaat gtgtaggata tggggtgacc caccttgttg |
| 21421 | tgcbgggggc aaagtccttc cattttggct gagctggtcc tgggggaacc catccatcct |
| 21481 | gtcttgatat agaaggtggg aagctctggg aatgggtgga gggggagaaa cagcttagga |
| 21541 | gccaagggcc cttgcaattg gtagtgctgc cttcaggaat tagatgatcc aggcccctgt |
| 21601 | cccttagcca gggaaagaac tggccatgtc tccaagcttg ctgcccagga gacaggagag |
| 21661 | agcbgttttt gtctgtgggg gtcttgttgg ctgcaacagg ctgggagtgg gagggggatg |
| 21721 | ctgctggagg gctgtgactc cagggtgtaa cttaatttta catagtttta caccctggag |
| 21781 | ttccttgagc ctctggaaga tggacccata ggtttggtca ccactgatgg gacactccct |
| 21841 | gccccagctt gccataagct cttctctcac gtcctgctcc tgggtaaggt ggcacctatc |
| 21901 | caggtctttg acactgaagg gcagtgcttc caaattcact tcctctagcc tctcatttat |
| 21961 | tcttgcaaac catagatatg gcttgaataa atattgagcc agtcattgtg ctgctatgaa |
| 22021 | taagacacaa ttccacccct caaggatctg gtgaggatgg gtgggtgggg agacccaaaa |
| 22081 | ctataaatcc atgagcagaa aaatacataa aatgtgctgg gggcatctga tctagcttgg |
| 22141 | gagtgggagt tgtattgggg gtggtggctg gggggtggtg tcttatgaca ttatctctag |
| 22201 | gctgccactt aaagtatggt ttgaagacag ggagaacggg gojgcggagt gaaagggttg |
| 22261 | aggacatccc aggcagaagg gatagtgtga gcaaggcatg aaggtggcac ttgggcaggg |
| 22321 | gcagggagtg ggtgggcagg gtggaggctg gagacacggg caagggttag ctccaaggga |
| 22381 | gcctgcagca ccatgccaag agcctagggc ctgtcaggga tgagcagcag ccgaggggtt |
| 22441 | ttaaacactg agojgctcag tcagatagac ttttccatag gtgcctggca acagtatggg |
| 22501 | aggcagagag gccggtagag gctgttgtgg tcttccagga ggaattgtgc tggtggtggg |
| 22561 | gaagcacctt tggtgactgg cagtgccggc cattgcaagg tctcagcctc aggcagcttt |
| 22621 | tggtctagta agtgctcctt tcccaatcac tgaccacgtg ccgaccccta cattggttaa |
| 22681 | cactgaatct gcacagcagc cctgggaggt aggtgctttt tttttttttt tttttttttt |
| 22741 | gagatggagt cttgctctgt cacccaggct ggagtacaat ggtgcgatct cgggtcactg |
| 22801 | caacctccgc ctcccaggtt caagggattc tccctcctca gcctcccgag tagctgggat |
| 22861 | tacaggcacg caccatcatg cccgactaat tttbitgttt tcgtagagac ggggtttcac |
| 22921 | catgttggcc aggctggcct caaacccttg acctcaggtg atctgcccgc cttggcctcc |
| 22981 | caaagtgctg gcattacaga tgtgagccac cgcgccojgc aggtaggggc ttttttcatc |
| 23041 | ccatctggat ttttgctgga gcctttaaat tggccgcctg gcttctcctc tggccttcct |
| 23101 | ttagttgaat ctcacacaac tgccagagtg agcctgttaa aatgtgagtt aagttttggc |
| 23161 | accttttgac tcaaaacctt ccagagatag ttttactcgg agaaaaaccc aaatggcctt |
| 23221 | aaaaggcctg caccatttgg gctgcctccc tgactactca atgacttcgg gc |
| Human VEGF-B Gene (NCBI Accession No. NG_029823.1) | |
| (SEQ ID NO: 81) | |
| 1 | gccgtcccog ccgccgctgc ccgccgccac cggccgcccg cccgccoggc tcctccggcc |
| 61 | gcctccgctg cgctgcgctg cgctgcctgc acccagggct cgggaggggg ccgcggagga |
| 121 | gtcgcccccc gcgccojgcc cccgcccgcc gcgccojggc ccgcgccatg gggctctggc |
| 181 | tgtcgccgcc ccccgcgccg cojggctagg gcgatgojgg cgccccojgc gggcggcccc |
| 241 | ggcgggcacc atgagccctc tgctccgccg cctgctgctc gccgcactcc tgcagctggc |
| 301 | ccccgcccag gtacgtgcgg cccgacagcg cgcccgcccg cccgccgtgc cctctctcaa |
| 361 | ggttggcgga agtgaggagg cgacccgcgg cctojgccga ggggatctgc ggggtccggc |
| 421 | cttggacgcg ggcgtctojg gggccoggcg cgtgcatccc ctgggaggtg cccctccccg |
| 481 | ctggcccgcc agccccagtc tggggccact cctccgcccc accccgtccc ctgggcaccg |
| 541 | cogggggcgg ggctccctcg gtgcctgcag gacctgcttc ggtgggcact gtgcgcgctc |
| 601 | ctgatctgca tgcagccccc aaacatgctc ggctccgcgg cctggagatt tggcgcggcc |
| 661 | accagagcac ctgttgcgtt ttgggccagg gcaggtcccg ggtggccagg agaggacagg |
| 721 | taaagctaga gcagtggccg caggaacaga gcaatctgga aagatgtcag agaggtggtg |
| 781 | aagcctactg atgcaagggc aaagccccag ggacggtgac agggccacac cctcctaaag |
| 841 | tgtaccttgg gtacaggtct tttctctccc acaggcccct gtctcccagc ctgatgcccc |
| 901 | tggccaccag aggaaaggta atacttacaa aaactojgca ctagccagca ctaagagggt |
| 961 | ttgtcatggg ccctgattcc aggtgtccat catcttgtgt aactccccta gaagattcaa |
| 1021 | actattattc tacccatttc acagaggagg aaattgaggc agtgggttta ctgggatctg |
| 1081 | gataaacagg gcaggggaag acaggttgtg agggcagagt ggggagggct agtggagggt |
| 1141 | ggctgtgact tggggactgg ggaggacaga tgctgggagc agctgcagga aaaccagtga |
| 1201 | ggacttaacc cctaccggtc tgctcccagt ggtgtcatgg atagatgtgt atactcgcgc |
| 1261 | tacctgccag cccojggagg tggtggtgcc cttgactgtg gagctcatgg gcaccgtggc |
| 1321 | caaacagctg gtgcccagct gcgtgactgt gcagcgctgt ggtggctgct gccctgacga |
| 1381 | tggcctggag tgbitgccca ctgggcagca ccaagtccgg atgcaggtac tgggcaggtg |
| 1441 | gggcaacggg caggggatgc aggtactggg caggtggggc agojggcagg gtggatgctg |
| 1501 | gctggctctg gggcbggggc agcctggaga ctgatgcaca ggagacaggg ttggggggag |
| 1561 | ggcbggtggc cagcctcccg gctgttgggt gagcttttct cccttcagtc ttagagcatc |
| 1621 | ccctttctct ctctccctca ctgtcccccc tgttcttctc ctgagcacag atcctcatga |
| 1681 | tccggtaccc gagcagtcag ctgggggaga tgtccctgga agaacacagc cagtgtgaat |
| 1741 | gcaggtgcca gccaggccca acttctgagc tcgcagaggc caggcttggg gggtgctggg |
| 1801 | tatggtggtc cacagaactg gggaccaggt tcctaagagt agaaccaagg ggccgggcgt |
| 1861 | ggtagctcac gcctgtaatc ccagcacttt gggaggccga ggcgggcgga tcatgaggtc |
| 1921 | aggagatcga gaccatcctg gctaacatgg taaaaacctg tctctactaa aaaaatacaa |
| 1981 | aaaattagcc gggcgtggtg gojggcacct gtagtcccag ctactccgga ggctgaggca |
| 2041 | ggagaatggc atgaacctgg gaggcgtagc ttgcagtgag ccaagatcgt gccactgcat |
| 2101 | tccagcctgg gcgacagagc aagactctgc cacaaaaaaa aaaaaaaaaa aaaaaaaaaa |
| 2161 | agagtagagc caagggtagg ccttggatct ggggcaacaa agtaggatgc ttgggccggg |
| 2221 | ggtagtggct cacacctata atcccagcac tttgggaggc ccaggcaggc agatcacctg |
| 2281 | aggtaaggcg tttgagacca gcctggccaa catggtgaaa ccccatctct actaaaaaaa |
| 2341 | aatacaaaaa ttagccgcgc atggtggcag gtgcctgtaa tcccagctac ttgggaggct |
| 2401 | gagacaagag aatagcttga acctgggagg cggaggttgc agtgagccga gaccacgcca |
| 2461 | ctgcactcca gcctgggcaa gaagagggaa acacagtctc aaagagagag agagagagag |
| 2521 | agagagagag agagagagag taggatgctg ggatttcctg atcttcctct tgtttgtctg |
| 2581 | tgtctgtcta tcttactttt cagacctaaa aaaaaggaca gtgcbgtgaa gccagacagg |
| 2641 | tgagtctttt ggactccagc tgagtagggg tatggggagt acaagtgagt ccagaagctg |
| 2701 | ttgctcctct ttctcctccc acccgtcccc cactttccct tttcctctgc tccccaagcc |
| 2761 | bitgttctct gccccgaccc cagctctagg gaagactctt ccttcccacc ccagacatgt |
| 2821 | cgcttctcct ccctagggct gccactcccc accaccgtcc ccagccccgt tctgttccgg |
| 2881 | gcbjggactc tgccccogga gcaccctccc cagctgacat cacccatccc actccagccc |
| 2941 | caggcccctc tgcccacgct gcacccagca ccaccagcgc cctgaccccc ggacctgccg |
| 3001 | ctgccgctgc cgacgccgca gcttcctccg ttgccaaggg cggggcttag agctcaaccc |
| 3061 | agacacctgc aggtgaggcg tctgtggggt ggtgtttgtt tgggaaggca ccaaggccct |
| 3121 | gtcctggagc aggtccaggg tgagcctgcc agtaggagga gggccaggga aggggaaact |
| 3181 | gttgaatgtg ttgaacaaat atttaccaaa cagctgctgg cacctggagc tgtataagca |
| 3241 | agtccaacat tctaactcag gagtcagata caggagggac aatgccagca gaggatgtca |
| 3301 | agggcbgtgg tgaggggtac ctggcctcgt ctttcagagg tcagagatct cttggaggag |
| 3361 | gtgacatctg ccoggagtag aaatgtagga cacagctttt ccagcttgca gttcgggcct |
| 3421 | tcccagaagc ccctgtggta gacatctgcc cttacctacg ggcttcaaga ggccagggga |
| 3481 | catagtggaa cccttgtcca cccttacctg abitgtttct caccatccgt gtcccgcagt |
| 3541 | gaggccatgg ggtgaaatca tatgggcagg gccagtgggg gctcccgagg agtgtcagga |
| 3601 | ggggatgacc tttgcaaaag gttcatcctt cgccctgcag cctccagtgc ccagggccga |
| 3661 | gtggtgtggc aggatgctca ggttgtgatc ttagtgggcc cgaggcacac atgagtccag |
| 3721 | ggcbjgggct gcgctccagc cagcatgaac agatcacttc ctccctcctc aggtgccgga |
| 3781 | agctgcgaag gtgacacatg gcttttcaga ctcagcaggg tgacttgcct cagaggctat |
| 3841 | atcccagtgg gggaacaaag aggagcctgg taaaaaacag ccaagccccc aagacctcag |
| 3901 | cccaggcaga agctgctcta ggacctgggc ctctcagagg gctcttctgc catcccttgt |
| 3961 | ctccctgagg ccatcatcaa acaggacaga gttggaagag gagactggga ggcagcaaga |
| 4021 | ggggtcacat accagctcag gggagaatgg agtactgtct cagtttctaa ccactctgtg |
| 4081 | caagtaagca tcttacaact ggctcttcct cccctcacta agaagaccca aacctctgca |
| 4141 | taatgggatt tgggctttgg tacaagaact gtgaccccca accctgataa aagagatgga |
| 4201 | aggagcbgtc cctgcctgtg tcactgtttg tcactgtcca ggctggctgg tttgggcatg |
| 4261 | aatgtctgca tcactaaatc cagagcttgt cttgctccct cattgtgcag atggaggaaa |
| 4321 | tgaggactaa ggccocacag cagatoccag gcagggccag aattatgtat tcatcacttt |
| 4381 | caagttattg ccacgcatgg gagtcaggga tagcccagtc aatacagact gcctgccctc |
| 4441 | ctgctcttca ccagggttct tttctagaag gagacagcct tctgtggcca gagagcttgg |
| 4501 | ggtaggaccc agatctactg agtgaccttg cttgtcacta cccctgcctc tctgagcagc |
| 4561 | agtttccaca tgtgcacata gagggaacag aagattgctg tggttggcgt cctcgggccc |
| 4621 | cagagaagtt tgagactatc tttacgtaat agaaaagaac acttgttctt cctgccaggc |
| 4681 | a |
| Human VEGF-C Gene (NCBI Accession No. NG_034216.1) | |
| (SEQ ID NO: 82) | |
| 1 | acttcgggga aggggaggga ggagggggac gagggctctg gcgggtttgg aggggctgaa |
| 61 | catcgcgggg tgttctggtg tcccccgccc cgcctctcca aaaagctaca ccgacgcgga |
| 121 | ccgcggcggc gtcctccctc gccctcgctt cacctcgcgg gctccgaatg cggggagctc |
| 181 | ggatgtccgg tttcctgtga ggcttttacc tgacacccgc cgcctttccc cggcactggc |
| 241 | tgggagggcg ccctgcaaag ttgggaacgc ggagcccogg acccgctccc gccgcctccg |
| 301 | gctcgcccag ggggggtcgc cgggaggagc cojggggaga gggaccagga ggggcccgcg |
| 361 | gcctcgcagg ggcgcccgcg cccccacccc tgcccccgcc agcggaccgg tcccccaccc |
| 421 | coggtccttc caccatgcac ttgctgggct tcttctctgt ggcgtgttct ctgctcgccg |
| 481 | ctgcgctgct ccojggtcct cgcgaggcgc ccgccgccgc cgccgccttc gagtccggac |
| 541 | tojacctctc ggacgcggag cccgacgcgg gcgaggccac ggtaggtctg cgttagggtt |
| 601 | tgcggagaac ccgagagttt gctttagggt ctgcggcgga cccgagaacc tgcgcggggg |
| 661 | aaagtgtgtg tgctttaagc ttgtgtacgt gggatccaaa gttactgagc tcagtgcacg |
| 721 | ctgctttgga gaaaaatctt cttcttttta aatagaaagt tgttactaga gaggcaagca |
| 781 | agttacacga gtgaagggcc cggagaggtg cccagtgaga gatcccgaaa tctatttcag |
| 841 | actggtttcc tctggggcaa ccaaggggtc ttgaaccctg cccagtcagc ggggctctgg |
| 901 | agagtatgag ttcattttgg tcgggaaatg ctcgtttctt tccccagctg attcatggga |
| 961 | ctccaaacag attctgggac actggtgatc agtcaaccca gccttacttt cctggagtgt |
| 1021 | tcatagtctg cagagcacca ggcgcbgtga gcgactttag aaaaaaagtg tcagggactt |
| 1081 | tagtaaccag gctccagagc tttcagagtt cacttgaagt tggtcagact tgagatgtaa |
| 1141 | cgggagatta gagtcagtta gatcacaatc aagcaatgca aggcttgttt caactttata |
| 1201 | agctcttcat tcctaaaatc tggtcatgag atactgcaaa ctaagttttt ttttttcttg |
| 1261 | tttgggtttt ttgtttgttt tgttttcttt taaataagag gtgtttaatc ctttgcctga |
| 1321 | aaatgttgcc aaaatacttt tgaggcatcc tgattttgaa aaaggatttg tgtgtgttcc |
| 1381 | tacttcttac tggctcttcc aaaagagtat atctttatct aaaaagttca tacctgccat |
| 1441 | aaccatatag agtatgctta agagagttcc taaagaagtt atattcgaca tttcagttca |
| 1501 | aacacttgca gtaccccttg ctggaactat actgoggtag tttatttcaa ctggtggcaa |
| 1561 | gtggagaggt acctgtggtg tgttacagtc aactttaatt tgactgttga ttaacacata |
| 1621 | cacaatgtgt agaaaatagc cttatatttt gaaatatttt atttatattt atattttgaa |
| 1681 | atacacttaa agattgagaa gactaatttt tggaaatcaa actacctttg catttaaatt |
| 1741 | ttgggaaaac attaaaatgt tggaatgtgt aataatttaa tataggggtt aaaggaatgc |
| 1801 | ctcttgagta aaaacaatat acatgaaata gaacagactg cattctgtga atcacaaaaa |
| 1861 | taattttcag tgcctatact tacattgccg taactatagt gatgaaaata tctttctgtt |
| 1921 | cttaaatacc gcagaaatgt aataatcggc tcaataacgc ttctgataat ttgagtcctt |
| 1981 | gattttgcag tacctatttg ctatttctgc aaagtcaaaa ctagtaaaag tatgatttag |
| 2041 | taaggcaaca tctctgtatt ggttacggtc acttactttg gtttacagta agaaaatgtg |
| 2101 | cagtagagag taaaatgaga aacattccct gataagccaa tagcctgtat gagagtcact |
| 2161 | cactgcbjgg tgagtctaga attaatgtac cttgagtgtg tgtgtgtgtg tgtttacgtg |
| 2221 | bittatgtat aggtatggga ttggaggatt aaaaaagtta acttattttt aactttcaaa |
| 2281 | agtgtcttcg ttggtgatgt aaaatttgcc attggtgata tggcaaattt taccattgct |
| 2341 | gtgaaaatca ctatgcattt tbitttgcaa atgtcgcttt tcatatttaa ccatttttat |
| 2401 | attctgtatg gtgaatatgg gttagtattc tgcatgtgat gtgggttcct ttcctatgtg |
| 2461 | atgaaacatc atttcccata tgaaacatca tctctgagtt ccatagacca caaagcagta |
| 2521 | tttcattgct ccattaaagt gtcgttcccc agtgggtgaa tagagccttg acagggcatg |
| 2581 | tgtcattaat gcattcagtg aaactgctgt gtccttgcta tgaaacatgt cacacaactg |
| 2641 | ccaagaaact gatatgaaga ctctatgtct ctccctagtg gcctgtacaa gcaccacatt |
| 2701 | tagatttaag cactagagaa gaactgggga gaaatcaatt gttaattttc tataactaca |
| 2761 | tgaaccccaa gaaaatattt cagaaggtaa acagaagatg ctaaatatct ataatcccaa |
| 2821 | tagatgcaat gttttcataa ctttcaccta ggaacttttt taatatttaa aaaatcaaaa |
| 2881 | atagcacaca aactatgaac caaggttagc catccagccc ttcagcccac ccgtaaagcc |
| 2941 | atgcaaaaga ttgtaggtaa aaatcacaaa tagaatgaag atctccttat tcctcgactt |
| 3001 | atatttgcat ttactggatt cctatttcta gttatttgtg gttgcatgac ttactgaaaa |
| 3061 | gtcttaaaat gtttctctga aggatgttaa cacaaacaag acagtgtcaa aaaagtaagg |
| 3121 | caggcaaaag taaaattaac tggacatgat gggaacaaaa gagaatagca agtctgaagc |
| 3181 | tgttctattt acaatttttc ttatagtata tgattatgtt tgtcaacatc tactcttata |
| 3241 | agagatttct gacattcctc tgagtgatgc tacttatatg aggttttaga tatagaaagt |
| 3301 | tcaattcatc tttcattttg atttccactc cattatagtc ttctgaacta tgaatatctg |
| 3361 | attgatagct atttattcac aagacagtta tcaaaattgc ttcatagaaa gcagttaatt |
| 3421 | actgatctta gtggcatatg tctaagaccc gtagatctct aatcctgtcc ttttcagttg |
| 3481 | agttgagaaa gacggagggt agtggattta gatttcattt tggacttcca gttctggttc |
| 3541 | cggttcctct tgagaggaag gtcctggcct cctagttatc tccatgtgtc caacccagaa |
| 3601 | tgcbgtttct cagctcttgc tggggttttc accaacagag gaactggcgt gagactaatg |
| 3661 | aaggtttccc gaatccctgg gactgtgctt gcctgcttgc ctttggcagt cctggggttg |
| 3721 | ttattgagag taggagttta taatcattat ctgtgatgtt cataacgtgt gttaagcact |
| 3781 | tcgcaaagag ctttgcaagt tagttttgtt tttcactaca cctccataat atggacatta |
| 3841 | ttttatttca ttaaggatga tgaacccaag atctgaatga cttaaccagg aaaaagaata |
| 3901 | gatgtttttt tccttttttt cttttaaaaa aagttttatg gcatatacca acagtccccg |
| 3961 | acttagaatg gttctattta ggacttttgg actttacaat ggtgcaaagt cgatagacat |
| 4021 | tcagtagaaa ccatacttca aggtttgaat tttgatottt toctgggcta ataatatgca |
| 4081 | gtaaaatact ttctctggat gctggacaat ggaagtgagc tgtgaccagt cagccatgtg |
| 4141 | atcgtgagag taaacaacag gtactctact gtatattaaa tatattttca actcaggagg |
| 4201 | aatttgtcag gatgtagccc catcgtaaat cgagaagcat gtatgtttta gagaaatgtg |
| 4261 | gtaatgggtt tgggcctttg ggcctgtgat tcagactcct ttcagcccct ctgctggtct |
| 4321 | cctttggcac actatagcag cgtgcaggaa ggcagagaaa agcactgcgt gtgactttgc |
| 4381 | agttaggggc accttgttgc atttatttag gtcctaattc cccacaacgg gacaaacgga |
| 4441 | aataaaccca gactttcttc caggctttac cacatggtgt tcgtttgact aaagataaat |
| 4501 | tataaatgac ctaatccatg gtaatattat aagaacgact gcttaactgg aggcttaaca |
| 4561 | agattttctg acaaacggtt tttcaaagtt gtgctcaagt aacttcagtg aaagattgtg |
| 4621 | attttacctc tgccaaatga cctttcacaa ataagtggat ttggaaagaa caaatttaag |
| 4681 | ttggcagaag cgttatactt cctctgactt ctgagcatat attgtttcac ctttcaccag |
| 4741 | ttacattatt ccttgtagat gtgcatttta taaatactat atgggtctac cttttgttac |
| 4801 | aaacacatat ttgtatgtac agatttgtat aaattgtgga ttaagttatc attgattttt |
| 4861 | ctcattagaa aaaacagata tagcagagaa ctttaaacaa ggagagatgc tgatgagttt |
| 4921 | gagttatagt gtaattataa aatttactat aaatatttta ggtttgtgta attgtttttc |
| 4981 | tgaaacttac tagtgtaaat ttttggggtg gcttctttcc toggttgatt aatttttttt |
| 5041 | ttcagagttt atgtttaacc atgagaaagc agagactgtt tttatttaat ttgctttatt |
| 5101 | gggcatccat catggaatta atcaaattgt acattaaatg ctttgtcagt ggattagtac |
| 5161 | tgtttcctta tactgctcgc gccctagtgg ctttcaggtg ttaggagtta tccacgatag |
| 5221 | ttttttttcc aattttgaaa tccaaaaact cattttcttt ttctttcttt cctttttttt |
| 5281 | tttttttttg agacagggtc atgctctatt gcccaggctg gagtgcagtg gcatgatgtt |
| 5341 | ggctcactgc aacctctgcc tcgtgggttc aagcgattct cgtgcctcag cctcccaagt |
| 5401 | agctggaatt acaggcatgt gccactagag caggctaatt tttgtatttt tcatagagag |
| 5461 | agggttttgt cttgtaggcg aagcbggttt caaactcctg acttcaggtg atccacttga |
| 5521 | tctcctaaag tgctgggatt acaggcatga gccactgcgc ccggccccaa aagctaattt |
| 5581 | tcttaaatta tttggtgcta taatcctgat ctgaactgac ataaggctat ttatagtcat |
| 5641 | tattatctaa tttaaatatt tctaatcttc actgggaaaa tagtaatgta tttatttaca |
| 5701 | tgatgctgcc atcagtcctg cttgtttttg gggtgtgagt attaatatgt ctatgcactt |
| 5761 | ggtttccaga aaaattccag gttcagagac tcttgcagtc tccaggcatt ttggataagc |
| 5821 | agatggattg tagccctgtt ccacaggcct tcctccattg ctttattgcc ttggggctgc |
| 5881 | ctggaagagg cagggaaagg taatccagga attcaagaaa gaagccatgt ggatgctcct |
| 5941 | aattcctttt tacccattag taccaggttt atttgctctt taatctactt caactcatgt |
| 6001 | taatcagttc ttactagtgg cagcttcagt tctaagcagg aaattacagt tcagtgtgtg |
| 6061 | atgcaaaaaa tgtagagtga gtgatcttgt ctataacaag cttataatca ttttggagaa |
| 6121 | aataaattta ctcattaaaa atactacaag tgaattaaac tataaatcca gaccatgtaa |
| 6181 | gtggttttgt aagagagtct tgtgagttac caccagttgt tggctagcct tctgggagtt |
| 6241 | gcatgggcag gcctgggaaa ggtggaatga tatgagaccc aatgttaaaa atgagtagat |
| 6301 | gctaattaca gaaatggggg agagagggat gttttgaata ttgaagaatg acagcaaagt |
| 6361 | acagaggaag taaaggtcag ggatatttgg ggaactagaa gcaaaccctt tggtgcaaaa |
| 6421 | gtcaagagct ttaagggaaa ctgaagggag catggtggga agtgtgaaaa ccaaagagaa |
| 6481 | agtctgagtt tgagttgaaa gcagcaggag ccaatgaaga ttctttagtg gtatgctgaa |
| 6541 | agtgaggttt ttaaggagct taacctggca ttgttgcaca gaagtgtcta agaaagctga |
| 6601 | ccagaagctt ctcagaagct tctcagaagc cattttcaac tgcagggttt cttaaagtgt |
| 6661 | ctggcagtga ctctgccttt ctcattacag tggccaccca tatatggtat tcgaactcct |
| 6721 | ctatatgctc ctgattgctg gcgatgatag tggtgattac ttttgactta ctgtaggcac |
| 6781 | ccatcattgt gctatctgga tacatttaca aaagtataga aatagacatc tctgccatta |
| 6841 | tggtttgcat tccaaacgaa gcaagcagtg taaacagtgg tcatgcttag atacacatac |
| 6901 | acagaacccc ccacacacat attcatgcat acaccgctta atagttcttc agactgccaa |
| 6961 | gtgtgactta cgtgttcttt cttgcatggt taagaatttg tagacagttc attgaaatag |
| 7021 | aatatttaga caatcagaag tgtacaagat tagaatctct atatttgcac acacatttat |
| 7081 | ttgtaggtca gtgggcaatt gaaaaaaaag agaaggagag aaccctaaag tgtcctggaa |
| 7141 | attcctgctt tttaaaatct gctaaaaatg agaccagaaa ggtgggagtg gggatgtgag |
| 7201 | gaggtgggta aactaccaaa taaacaatat aaatactttg gcbgttctta taaaaggttt |
| 7261 | ttttaaatat ggtggaataa tttactcaaa ctcaagagat gccacctact agagaaagga |
| 7321 | catactgaaa gaggaaatat tcaaatgcac aactttgtgt aaaaggtaat acttacaagt |
| 7381 | ttaaaagact cacttctaaa gaagtttggc ttcaacctgt cctcatttgg acaggtggaa |
| 7441 | agatgtatat tttggggtag accagaaaga tgtggtttga cttctatagt tgaaaggttt |
| 7501 | ctatttagac atttattttg taatttaatt tacctaaaac ttcatcccta aattaccatt |
| 7561 | ttctcttact tttatgcaat agtaaagtat gcagtcataa gtgataaagg ctgggatcaa |
| 7621 | agctagctat tttgctaact gcagaaccta tgcactctgt ttacactgcc tgtgatagcg |
| 7681 | cctggaagaa ctcactgcca atatttctgc ttgtttcagt cacataactg ttgcttgtgt |
| 7741 | cagtgacgta acttgtttag agatgcagtg cagtggataa gagcacaggg tatggacatg |
| 7801 | cctttctgta ttcatgtcct gaggatgcct cttagtacct gtgtgacctt ggacaagtta |
| 7861 | cttagtttct ctctgcttct ttgtagcaca atgagtagat gctaattttt aatttttaat |
| 7921 | tttttctttt tcttbitgtc tgttttgtgt atttagttac tttatctttt tttttttttt |
| 7981 | tttttttttg agacagactc tggctctgta gcccaggctg gatggaatgc agtggtgcta |
| 8041 | tctcagctca ctgcaagctc cgcctcccag gttcacgcca ttctcctgcc tcagcctcct |
| 8101 | gagtagctgg aactacaggt gcccaccacc acacccagct aatttttttt ttatattttt |
| 8161 | ttagtagaga cggggtttga ccatgttagc cagaatggtc tcaatctcct gacctcgtga |
| 8221 | tctgcccgcc tojgcctccc aaagtgctgg gattacaggt gtgaaccacc acgcccagcc |
| 8281 | tagttacttt atccttttaa aagtcctctt ccaaaatgta gaatataatt aatgcctgct |
| 8341 | taatcaggtt gttttgagga taagaatgtg tatataggcc aggtgtggtg gctcacgtct |
| 8401 | gtaatcccag cactttggga gtctgagtca ggtagatcac ttgaacccag gagtttgaga |
| 8461 | ccagcctgag caacatgggg aaaaccctat ctctacaaaa aatagaaaaa ttaactgggc |
| 8521 | atggtgacac gtgcctgtat tcacagctac tctggaggct gaggtgagaa gatggcttga |
| 8581 | goccaggagg cggaggttgc agtgagtgag ctgagatcac cccactgcac tccagcctgg |
| 8641 | gcaacagagt gagactctgt ttcaaaaaaa aaaaaaaaaa aacagcatgt gcatacatat |
| 8701 | aaagcccttg gaaccatgat tggcatatag taagctttct gtaaatttgt gttaaataga |
| 8761 | tagtcttcat gatcagaaaa caaacaaaga atatagcatg taagtactat gtcctagaca |
| 8821 | tatgacataa ctgtgattct tcagcacctt gcaactaatt tattaaattt ttaaaattat |
| 8881 | aaattactga caattttccc aatttctaaa atatagcaag aggatataat actgatgttt |
| 8941 | gattttgaaa caggtgaagg aaatgcaggg tgtttaatca ttcagtgtct ggaggctaca |
| 9001 | tactgtagtt ctgcctataa tatgttccca cttcccctgg gtgccggggt ttgttcctct |
| 9061 | cctggagggc agcacaggtg tccatagacc acaccaaacc aaagaatctg ggatgatgct |
| 9121 | gtctccttat gtgcttcatt catacttagg tcatttggct gtaaaggatt ttggtacaca |
| 9181 | taagcttttt ataatgccat ataatttaat atgaactcat cttgaatata ttcagagcat |
| 9241 | atacattttc aagtttaaat gtttattaat gaaacatgct aatgttgcta ctttgatgtg |
| 9301 | taaatatctc atctatatta ttgccacaaa aaataaatat gcattctaaa tttacaatga |
| 9361 | aatcatcaca gaatacctaa aatatcaaca atttcactga ggaaaatata aagaaaactt |
| 9421 | aaggggagaa tgctatatgc cagagttaaa cttgtgtgaa tttgtttagg aaaatgtgat |
| 9481 | tgcactgttc ctaagggacc ctcttccaga atggtaacta gtacagcgtg tgctgtctga |
| 9541 | gatgtgcgga cactgggaca ctgggatgaa ttatggagta gatactttcc ttctctgaga |
| 9601 | gtaagctgat ttgcttgagt actctagttt attccaaata gttgattttg tttggtggtt |
| 9661 | ctctttgtca atttgccagt cagtgctgtg acttgcgtgg ttaccctgtg ctgaagacta |
| 9721 | cagtgcbjgg ggcattggag gcccgtgagc agacaggaaa aagggaggaa aaatcagtgg |
| 9781 | tggacacact tcccagctct tgaacagctg acagcatatt tttttgattc agtaacatcc |
| 9841 | agaaagtatt cacttcccta ggattccagt gcagatgtta gttggagtat tttagaactt |
| 9901 | ttgaccagat accacatttt taaacagaat tttttttctg actggaactg gaacaaagca |
| 9961 | ctaaacacta aatagaccct caggtagtat gtaagtacat taaaaattag ataatactat |
| 10021 | attattcatt cacatctcta tggagatact gtatcatctt tttaaaagca attttcgtat |
| 10081 | taatatttgt atttcagaat tagaattgta tgtaagcata taataaaatg aagctttata |
| 10141 | gttcagtgct attatgtttt cccaattata gaattcaaat taacatttta atgtgatttt |
| 10201 | cttcatagta atttttcatt gtcacactaa tttatttttc tctcaacttt tttttattta |
| 10261 | aaaatttttt ttttttttaa tttgagacag gcctcactct atttcccagg ctgcagtgca |
| 10321 | gtggcatgaa cgtggctcgt tgcagcctcc gcctcctgta ctcaagtgat ccacctacct |
| 10381 | cagcttccca agtagcbjgg atcacaggca cgtgccaccc cacccagcta ttttttaaaa |
| 10441 | ttttttgtag aaatgaggtc tcactaagtt gcccaggctg ctctcaaact cctgggctca |
| 10501 | agtgatctgc cttggcctcc caaagtgctg ggattacagg catgagccgc tgttccagcc |
| 10561 | taatttcttt ttctcttaca agtgctttgc ttacattttt attgttagaa acaggattca |
| 10621 | aataactgct tatttaatat tctttttatg tagaagaaac agtcttgaac ttgataatac |
| 10681 | ttttggttcc aacttgttaa aatattggaa aaggatgaaa catttgtgaa aataatcaga |
| 10741 | ccttggcact goggtagttg aggaagagtc atgcgtgtga gtgagtgcca ctctgagaca |
| 10801 | ggcagcactt tctcccttgt ggtaattgaa tagggtatgg gaagccgact acttaatctg |
| 10861 | cttcaggcaa gcagtgttca aagtttaacg ccottttact gaaatgacaa acataatcac |
| 10921 | cttacctaaa aggatgtgtg ggtaatgatt ttaccaaata gttcacttgg tgaacaatag |
| 10981 | tgaaaacagt tcaccttccc ttgtctttgt ttacaattaa atgagcccct ccaggtccct |
| 11041 | ccctgcccca agagtggatg aatttggcaa atcagactga aatgttaaag aaaatggtct |
| 11101 | tgactactct gttttaactg aagtgaccac acagctgcca tgaatcctgt gattgaattc |
| 11161 | ctataacctg taaaagggat tgttatttcc actcactcca aatttcttgg aagtagctag |
| 11221 | ttgatatcag ataccagatg cgaggctatt ttaaatcact gtgatttaat ttactataat |
| 11281 | atcattgcct acatggaata tttttaattc tttctttttg tttgtgtctg ttttgtgtat |
| 11341 | ttagttactt tatcctttta aaagtcctct tccaagttta tgttttgaaa aaagagaaca |
| 11401 | cttactgaga ttcatgtttg cttacaggtc atgacattta tgacttccct aaaaaacaaa |
| 11461 | ctgatccctt tgtctcagag ccaaatttta gttttaacat aatagtgatt cattgtttgt |
| 11521 | tctctctgag tcctagatct ctcttttaat tttggtgtct gtccatgcca aattagaatt |
| 11581 | ttattaaggc caaaattatt catgagtcat gaccatcaca cttaacaatt gggtaatttg |
| 11641 | aagctgcttc agatacacat taaagcaaat aatgatgtca taaattttaa tgcaatattt |
| 11701 | tttttccttt aatattggta gcagtgaagc ctgctctgtt agagaccaat tctgttttac |
| 11761 | tttagaaaaa tgtacctttg tgctcatgtc catgagagca cagtgtagtt attttattac |
| 11821 | tataatatag cacacagtga cacataactt agtaaattaa agattgaaaa tatttaaaat |
| 11881 | tggtgatttt ttgtgccatg ttgatttgac atttattctt ctatctgtct aaaactagga |
| 11941 | gcagtgctag tgtgacagcc tcaagtcatg ttaacacgac acacgtagaa gactgacgct |
| 12001 | cgggcttcct ctagtgcaaa acacctgaaa ttcaaatttt gcttttctgt gctattttcc |
| 12061 | taattaaatg tatccttgat gttgagttta actttattac acccctgtta aatatctgca |
| 12121 | atgtgttggg aaggaggagg acacatatgc ctgatttaaa tttaatataa ctgtatagat |
| 12181 | ataatttcat tttccattca atcattgaaa tgatataaat taatatatgt acagctaaaa |
| 12241 | ttaaatctgt gatgtcgaga tgaactccat gctgtccttg gtgcactgtg tattacacat |
| 12301 | gggtattaaa tgcgtgctta gtgcttagtg aatgggttag aaggagtttt tttttttttt |
| 12361 | ttttgagatg gagtctcgct ctgtcgccca ggctggagtg cagtggggta atctcggctc |
| 12421 | actgcaacct ccgcctcccg ggttcaagca attctccctg cttcagcctt ctgagtagct |
| 12481 | gggattacag gcacgcacca ccatgccagg ctaatttttg tattatttag tagagactgg |
| 12541 | gtttcaccct gttggccagg ctggtcttga acacctgacc tcaggtgacc tgcccgcctc |
| 12601 | tgcctcccaa agtgttggga ttacaggcgt gagccaccac acctggccta gaaaagagtt |
| 12661 | ttaatagata cccctgtaaa atgccaagca atcagttgcc attactaaga actgcaacaa |
| 12721 | gttccagtta aagatgtaca tgtttaagag ctgtcgttct ttaataaaat atgaccaaga |
| 12781 | tgtcttaaac atgggcttta atacatgtta ctgtgggttt cactccaact taatagcaca |
| 12841 | caaagttctg aagacacatg atttcactgg tggtacattc gcacattgtt ttctgtccat |
| 12901 | gtttaagttc attcttgtcc agaacagttt ctggttttct aagcctaacg tattaacatt |
| 12961 | tgagttaatt aaatcgatag ggagcctttt atcttttttt ttttcctatc tgggggcttt |
| 13021 | gctttcgcac tgacattagc agttcccttc ctatgtgcaa taggaaaatg tgcagtcatt |
| 13081 | ccaggagcat ggtctcctca ggaggttaaa agtgattgta acttagaggt taatgtttca |
| 13141 | ctccagagga aagtgtttca coccacttga tgatgtgocc ctgggtgctc acagaaagag |
| 13201 | tagtactctt ttttaaacca aaattttaga cacgtgttat gcaaatcttc tttataaatt |
| 13261 | acacattctt ttctttcctt ttattctttt actccagtat actattttgc aattaaaaca |
| 13321 | ttttaaactt cttccagaaa aaaacttctt ccaataactt cttacttcaa actactttta |
| 13381 | ctaatgacaa aataactatg tccatcacag tgaggaatta ttcattgttt ataccacctt |
| 13441 | gtattactta tataaatatc atcctataaa aaatactata tattgggact agattttctt |
| 13501 | aaatttaaat ttaaattctt ttggaaaatc aggttctatc agctctgttg tcttttcatc |
| 13561 | tctaattgta catcttccca ctattttgat ttatacatga tgttattata ttaggatagc |
| 13621 | tttgatttta atgtaatatt agctagtttt atggcttgaa aaacttagta catagactat |
| 13681 | attcataaat acagcactat acattctagc atatggcgtg tgcacttaat ctgaagaatt |
| 13741 | agacattttt tcctgttctg tgatgattga ttgattggtg gtgggggcag aaggtggcca |
| 13801 | gtctttggca acatgtaaaa ttttgttaaa attttgtcaa actgatttat tccattgcta |
| 13861 | aattttgaaa tcagcatttt tttaggggga atatctcgag tttatgagga cagaaagaaa |
| 13921 | tattcttaga aaattgagaa ggttttgtcc ctgtacatgc aaactagggg gtgtttagat |
| 13981 | cagagcaaat gtcttgcagc aggggttgac gccgggccat gtttagtcat caaagcatga |
| 14041 | cttctattct gggaatgtgg taattgagag gtaaatatta tttacaatag cccttataaa |
| 14101 | tctttagttg ttcaacaaaa taacttgcca tcagagttag atgatttagc taaaattatt |
| 14161 | taattgggtt attcctactt gatccaacat ttgattataa tatatgtagt agcgttaaat |
| 14221 | aatttgggat ttctggcctc tggctggctg ttgaagactt tttgttggtg gcggggggca |
| 14281 | tcctaaggaa caacttgccc tctactagca ttctttacta gaaaaagctc ctatttactt |
| 14341 | gttttttttt tcctaaatat gtgttttatt tccttttggc attgtctttc ttgctcccaa |
| 14401 | ttataattgt gagcaaatag caatcgaaat tctgtgtaac ttccagcaca gtttagtcat |
| 14461 | cagagcttgc cagcaagtat gtctattgtg aggaaactgc atttgccaca gctagaccat |
| 14521 | cagcaagtga agtcgtattg tcctctgttt tgtagttgtt ttttcctatt tatgctttac |
| 14581 | tctgattcaa gttcagcatc tcagtgatca taattgtggg gaaaaaaatt cattttgaac |
| 14641 | tataaacaga aaagatacca agttgttgta tttgaaagat tattttagaa tgtggttata |
| 14701 | ccttcaagta aaataagctg taaaataagc tgaagaacca tatatttagg cacattttga |
| 14761 | ctataaaata gatcctctac atataacaag aatagctttt tttttttttt tttttttttt |
| 14821 | tttttttgag acaaagtctt gctctgtcgc ccaggctgga gtgcagtggc acaatcttgg |
| 14881 | ctcactgcaa gctccacctc ctgggttcat gccattctcc tgccccagcc tcccgagtag |
| 14941 | cagggactac gggtgcccac caccatgccc agctaagttt ttgtattttt aatagagaca |
| 15001 | gggtttcaat gttaaccagg atggtctcga tctcctgacc ttgtgatccg cctgcctcgg |
| 15061 | cctcccaaag tgctgggatt acaggtgtga gccacggagc ctggcctata tataacaaga |
| 15121 | attgctttta tatgtctata tgtcacttca taatatttgt ttattattta gctagttatt |
| 15181 | gcctataagc cagtatttag gtattatata aaatctgagg aaaacagttc tataagattc |
| 15241 | agtaaaattc caattctata agattccaag atgtaaagtg ttttaggtat ttaaaagttt |
| 15301 | tatatagaca atagtgactc atttgttatg gtatatgaat atagacaaga acttagactg |
| 15361 | aagcaaaaaa taacaattag ttacatagct atttcaaagc ccatgcagta gttttttgtt |
| 15421 | tgtttgtttg agacggagtc tcgcbitctc ggccaggctg gagtgcagtg gtgcgatctc |
| 15481 | ggctcactgc aagctccgcc tcctgggttc aagccattgt cctgcctcag cctcctgagt |
| 15541 | agcbjggact acaggcgccc gccaccacgc cojgctaatt ttttgtattt ttttttagta |
| 15601 | gagatggggt ttcaccatgt tagccaggat ggtctggatc tcctgacctt gtgatccacc |
| 15661 | cacctojgcc tcccaaagtg ctgggattac aggcgtgagc taccgcaccc ggccagtagt |
| 15721 | ttttaatgtt cagtgaagtt aaaacatctc ccgtccagtt atgctgtgta ataaatatta |
| 15781 | aagcaggaga tattagaaca atacaacact tactgtgcta tttataattt aaaatattaa |
| 15841 | agataaataa tggaataact ataaatattg atactatgat aaaactttta gcatgtctaa |
| 15901 | gttgttcatt ttgatagata caaaaaacca aagccaagca aaataaaaca cacctaaaac |
| 15961 | tttatgaatg ttaacaattt tctatttttg ccataaatct ttggccgttg caccatattt |
| 16021 | tattgaataa aggacagttg ttaagctagg gcttggtcca ctgbitgtca gtatcaggat |
| 16081 | gtgatgattc ttttctgacc catattcttc tctctctttg gctttggttc tcttgtacga |
| 16141 | ctcttctgta ggtggccttg gccacacctg tgaaatatag ggcacagttg tgctgcttct |
| 16201 | actcattgtg aacttgatca tabittttgt attgtgcctc tcttcattgg ctccttttta |
| 16261 | ttttagtgta attcaaagtg ttaattggag gttttaaatg tagttttgac caaattgtgt |
| 16321 | ttgaggtcac atgtacccag tttaaaaaac gaagaaaaaa attcaactca cttcccagcg |
| 16381 | tcctttgcag ctaatgttgc cctcgaatag ttcttgctca taagagaaaa tctagcaggg |
| 16441 | atttatggga aaacttttgc ttttttttgc taaggcactg cttttcttct tgttccctta |
| 16501 | tttccttcca tggaaatcag aaaatgtttt ataattttaa aactttacct ttgtttctca |
| 16561 | aaataacact ttcagactta tgtaaagcaa gattttaata attaatatgt tgcctcgatt |
| 16621 | atggagtaac tttccaaagt tgtactatca tgatcttaat ttattgaccc ttagggttaa |
| 16681 | gataatttgt gtgtttataa cctaacattc tttgcttggc tgatagattc ctttaaaatg |
| 16741 | ttabitgttt atattttgta catactttaa gaagctttat tcaattgcca catttcaaat |
| 16801 | aaataaaggt tctttttttt ttggtaagcc tatacctttg tttcttctag cttttaaaag |
| 16861 | tgttaatagc gtgggaatga cccttgacca tctctgattg tttgggaaat ggtaactaga |
| 16921 | aagaactgag agaggacaaa cttggatgag aagtctcaga actgatagta aaaacaaatg |
| 16981 | caaacaagaa aatatattac gtacttcatt cagattaata agtaattaaa cacttgaact |
| 17041 | attcaaaaat ctagtttgag tctaggcttt tgcaagcctg aatttgtcat cataatcatc |
| 17101 | aaaattagaa attacgaaag aatgttaatt taaatttaaa acagctggaa atacaataca |
| 17161 | tggcctataa gcttatcttc tcatctgtca acaactgctt gcatatgtca catgaactgt |
| 17221 | tttaactgca attacaaaag tcaatggaat gtttagaggt gattcaaaaa atgtgattgg |
| 17281 | ctgacatata gagttatcct aagaaacagg ctgtccaaca caattttaaa tatactcaag |
| 17341 | ctcttttata ctgattaaga gtttcaagtt aaggttttgg gttaaaaaat caacatcaaa |
| 17401 | tgataaataa gaaatataat cttgtttaaa ttatttattc taatagttta tttatgaaca |
| 17461 | caacatattt ctctttcatt actttttttt tctcagttgt aaaattctag agctggagcc |
| 17521 | cataggccaa tccgtagtat atctgaaatg attaaaggtt ttatacttga caagccctct |
| 17581 | gtctgcaggt tacttgcttc tacttggagc agtggattta tcatcctcca ctccagggta |
| 17641 | aatatctaaa agtcacttct gccacgttat ttcccatctg gtagattgtt ataatacctg |
| 17701 | tgagtcagta ttatctgcac agtgtatttc ctotgctcaa ggcactttga ccatatgagg |
| 17761 | tgttcacatc tgatcaatcg ggctgaagct tgccctagtc tgaggctgga ccaattgatc |
| 17821 | tctaacgata ttcttacctg acaatctaga ggatacaaat caagatgttt caagtgcaga |
| 17881 | aagcaacagt aaagcataag ttcttttttt ttttttgcat tctttttttt tttattatta |
| 17941 | tactttaagt tttagggtac atgtgcacaa tgtgcaggtt agttacatat gtatacaagt |
| 18001 | gccatgctgg tgcgctgcac ccactaacaa gcatatgttc taatgtgtaa tatatcttca |
| 18061 | agacagtctg gaccatgaag tgtattttta cttattgagt gtatatggga ggatcatttt |
| 18121 | ataggattgg ttatattttt ccatttcttg attctttgat acttaaattt ttggtttcat |
| 18181 | attctcctct ttcacatgtt gtccccattt tacatatgca aagatagttg tttgccacaa |
| 18241 | aaataaacat ttaacagact ccaaacacat agactgtgta attagcataa cgaaataata |
| 18301 | gtggctgagt gacgagcaga agtgattcac tcatttggct ctgtggcttt caaagttatt |
| 18361 | aatgatgtta catattcttc tctattttat ttaaaaacta acactcataa agttaattct |
| 18421 | cataattttt gcbjggttta ataattcaaa atatgaatca aaatttttat ttattatgca |
| 18481 | gtttcattct attaaaatta tctgctaaat taatattaag tagtcctata gcatatatta |
| 18541 | tttaataatt gcaagtagtg acatatcata aataaactgt ataatatgta ttattgattc |
| 18601 | tgttattttt cctagcaatg cacagggaac cagtaaattt cacaagcaga gaatactaac |
| 18661 | ttgtcattta tttaatattc taaacaaatg aagccgcctc tataagtgaa ttttctggac |
| 18721 | ttctaaagat gagcattgtt gagtttaata actcaaattt ttattgtgtt aagtaaagta |
| 18781 | tattaaatat aacctcaccc taatgactca gctgtaatta aaaaagagtt cacgaccagc |
| 18841 | ctgggtaaca cggtgagacc ccatctctac aaaaataaaa aataaaaatg aaaattaaaa |
| 18901 | aaaattagcc aggcatggtg gcatataccc aagtactccg aaggccgagg gtggaggatc |
| 18961 | gctcaaacct aggagtccaa ggctgtagtg acctgtgata gtgccactgt actccagcct |
| 19021 | gggaaacaga gcaagaccct gtctcttaaa aaacaacaac aaacctacac atgaaaatta |
| 19081 | ttgctgcttc ccatcaattt tcagtgctat ctttatttaa cctctctact atttgccttc |
| 19141 | actttttaaa attttgcttt tattcttctt tctttggtct ccttctctaa aatttttccc |
| 19201 | ctgcttctgt ttttttgtat actaggaaga catttattat ttttataggt atgatcaaat |
| 19261 | tgtaaaataa gtttatttca ttctctggtg atttcactaa ctattttgag agtatcagtt |
| 19321 | aaaatgggat ttgtttgtgt gtaacaaaat ctaactgtag tggctaaagc aaattggagt |
| 19381 | ttatttttct ctagtaacta aaaggcccca gtgggcgttt aagggctggt accacatgga |
| 19441 | gcaacatcat atcatcaatg tccagactcc tgtgcttttt tcacttgatc atttttagag |
| 19501 | tttggccttc tgtgcttcat tcctcgtggt cacaacatgg ctgattgttg tgcagcttta |
| 19561 | tgtctgattt caaggaagaa agaatagtga gagaggaggt tgttgcctta acacattcaa |
| 19621 | accaaagcaa aagttttctc tgaaatcccc agtagatttg cccttacatc ttctcaggta |
| 19681 | gaagtgcatt tttgccattc ctggctgtaa gagaagccga taaagaaaat acatgcactt |
| 19741 | tctaacaata ttgaaattct tttcacaaag aaggaaagga gaatgaatgt actttgggta |
| 19801 | ggtatcgtgg tctgttttat gttgctgtaa atcacaaact ggatgattta taataaaaag |
| 19861 | aaattagttt tcctcacaat tctgaagact ggaaagtcca gggttgcagg gctgtgtctg |
| 19921 | gtgagggcct tcttactgtg tcttctcatg gtggaaggca tcacatcagg agagatttct |
| 19981 | ccagagtgca agaacaggtg gaactttata taacaaactc attcctgtga taaggagccc |
| 20041 | actcccaaga taataccatt aatccattca ggtgggcaga gcctcatgac ctaatcacct |
| 20101 | tctgaaggtc ccacctttca atgcttttgc attggggatt aaatttccaa cacatgaagt |
| 20161 | ttggggaaca cattcacgct gtagcaggag gcaaccacat ttgtgacatt gcgtcattgt |
| 20221 | actttctaga tttgttttct ccaggttgtt tgcaacgttg atgtactctt tgtataccca |
| 20281 | cctgaaagat taatgttact gaagtattgt gtttaagaaa gaaagatgaa tgctaccatg |
| 20341 | taagaccttt tgactgtcca aaagattaaa attccagttt ggagaggtag cacttacatg |
| 20401 | aaaaccttat aaaacatgag aacttaaact tgctaatttc attgtgtcaa atcgcataac |
| 20461 | agagaaagga aaaacttgaa ggccactcct cttgaaaaag tatttgagta gtgtcgattt |
| 20521 | cagtgtaaga ccaattaaat aatgtcaaat taagtaaata taacattaaa atattttctt |
| 20581 | taacatattg gtgttgattt cacttttact ctgtaatttt tgtgacattg gaaacaggaa |
| 20641 | atattcaaat tttgccttct tttggatcgt ttcctctccg tttcttaatg tgtaaaccgt |
| 20701 | aatagggctt tctgcagcca acgtgctcat tcttttcttt ctgttaggct gtgccttttc |
| 20761 | tgagaacagt tggtactgat ggagatttct gttctttttc ttcacacaag tgtcatatga |
| 20821 | aaattggggg atcagctatt ttcagatcac aggctcctct gataacagaa taccataatt |
| 20881 | ccaactttcc tgtgttgatt tgtagatccc acactgtgtg atcatgacaa acatctaagc |
| 20941 | ataagagggc gatgaatgat taaaatttat tcactcttcc cataaaacac ctggtacaga |
| 21001 | tgcagtgtgt tgtcctcaca gaggaaaaac agtggttcct ccttccaagt ggcgtgtcca |
| 21061 | cagcactggt accagataac aagaacctta gacttttctt gttgctgcag tgtgtctacc |
| 21121 | tgtgcctctc ttaagtgtct tacacgttac tcacagagaa agaaatacaa aaatggaaaa |
| 21181 | cataagctca caaaaatgca tttagtgcat ttatgatgtt aatggaaacc ttggatacag |
| 21241 | tcctctgtta tttccttact actcttgata ttttacaata ttgctttatt attcaaattc |
| 21301 | ataatcatat atgtgttaaa caattttttt tggtaagtag actgtgtatc aaacttaaga |
| 21361 | gggatttgac atggaaagac atttaaaaga tggcagaaaa cagctattat aagggcattt |
| 21421 | gaactttaga tattttccgt tggtcagttt ccatagatca tcatctgatt cactagggtt |
| 21481 | tggccagttt atattattta aattgtttcc cctgcaactg tagtattttc tttaatgttt |
| 21541 | tgaattattc ttaatgtgac ttgaataagg gtaattgtca catatgctga tgttttccca |
| 21601 | ttaggatgaa aatatttaga aaagctgtac tagcaaaaac caggttaact tgttttcaaa |
| 21661 | gtagaattta attattttat ttcaagcttt gattcctttt ttaaaaatta agtaaatgtc |
| 21721 | cttgagagca atacctttgt gtgtgtgtgt gtgtgtgtgt gtgtgtatca gttacttgca |
| 21781 | ccttcaattg aatgaaagac agtagtaggt tgtattatgg cagaggggta gaagaatgga |
| 21841 | cattctcatg ggtatctcaa aggtcaagtc agaattggat tccctgtgga tggaagagag |
| 21901 | gagtggagaa ggtgcttctc atgtggaaaa acaagaaaag tctatgacag gcctcagtgt |
| 21961 | ttgccagctg cagtggaatt ctcatagctt gtattgaact ctgtgtccta atcttctgaa |
| 22021 | accacctcaa gagagtatat attgctaaaa taggagctca gctatttttc aggggcagtg |
| 22081 | atccagagga cactataaca gaaatagata atttcaaagt cgaaaatata ttagatacta |
| 22141 | tttttattct ctatctggaa agtctgtata gaggctttat tctggtctgc tttctcagat |
| 22201 | aaatatccta ttcaggtcac tggaaataat atgtcaggtt ttaaaagaga cttcatatgt |
| 22261 | agccaaataa acctgctaag catatttaga aaatcatgct agtgactagt gtcttaaact |
| 22321 | gtttccacaa aaacaatgct gttaatagta ggatttaccc ttttgcattg gaaatgagtt |
| 22381 | gtcaaggtta aaaagatgag tagccagata ttcttgctaa acatgttttg agaagaaatt |
| 22441 | taataatata aagtttttgt taaatgtggc ctattaattt taaacaataa tactgactga |
| 22501 | agtcattagg tagctgtgga aagaagttag cctggttttt actaatgtag cttttctaaa |
| 22561 | tattttctgc ctaatgagaa aagagggata agatccaact acaattggaa gcttaaatat |
| 22621 | ttaatcaagt ttgcagaact actattaaga tcatatgtaa ctatcagata ttttttaatc |
| 22681 | tgaggacttc tttctagtaa atagaaattt cttcccaagc attataggaa tatgtcttat |
| 22741 | tgacttattt ttttggcatg ggttactagg aaataatatt ttcagggagt tcagggaccc |
| 22801 | agatttgatt cccagataat tcagattaac cttctttaat cattctattt tatgttatta |
| 22861 | ctggagagta cagggtaggt tgaaacagca taaactgcca aatcagaact ggatttgaat |
| 22921 | ttagtgatgg gcctcaggag ctgtatgacc ttaattctgt gagtcaggct ttctgagtca |
| 22981 | ggctgtgaac tgggcaaaat agtgtatacg ttacaacatt gtaacagagc tcatttaaag |
| 23041 | cattgtctca tgttagctac tgataaagca ttagttccca ctccagggcc tgttagaatt |
| 23101 | ctaactccat ccctgtgagt catgttcatg ctttcgtggt tctcatttga ggttttgtga |
| 23161 | ccaaaggccc cacagatgga ataaaattaa tctaaatagc catttattaa tgtaatatct |
| 23221 | gctagctaat tacatattta ttaaatataa ataacaaata gctccaagat accagtgacc |
| 23281 | tccatgttgc taaagccaac agttattaga acttctctgt tctgtgagct tttctgtgat |
| 23341 | ggtcacaggt gagaccgcaa gaggacacac aggagaggca caatgaaaca aagtttatta |
| 23401 | tactcatagg tcttagacag gaggtcaccg catgccacac agggtcacag ggtgtcttag |
| 23461 | tttgttcagc ctgctataac aaagtgtctt agactggata atttataaag aacagaaaat |
| 23521 | tcattgttca tagttctgga ggttgggaag tccaagatca aggagccagc agattgggca |
| 23581 | tctgctgagg gcccactctt catagatggt gccttgttgc acgtccccct cacatggtga |
| 23641 | taggggtaag gaggaagcag gctctcccaa gcctctttta taagggtacc aatttattct |
| 23701 | tgagggctcc accctcatga cctcatcatc tcccaaaggt ctggcctctt aataccaaaa |
| 23761 | cgcbjgggtt gatattttaa catgtgaatt ttggagggac attgacatta cagaccacag |
| 23821 | cacggggggt ggggggtggt tctccatgtg gcagaagaca ggaaagagga gagagcttat |
| 23881 | gccacagcct ttactggagt ttcagggtaa aggcaaggca gagtagagta gggtaaacag |
| 23941 | tttagaactg gctggtttga atactttcag taggctctaa ggtataggat taatccctag |
| 24001 | ttgcttgata cctgacagtg ggaaattaag gcagaggaat attgtctcct ggggtatata |
| 24061 | gccagataaa ggaggtatgg ctctggattg ggtaatttgc ataccaaagg tgttctccct |
| 24121 | gctgaggttt ttgctgtctg taaggattgg cagcccctgg agggccatct ctcccaagcc |
| 24181 | ataaactttt ttttaagatg tojaaatatc atagtactca gaaaaatttg aaatatatat |
| 24241 | atatatatat atatgtataa aacatcctct tacttggcag catttacaat gtttgatcat |
| 24301 | tatttttcag ctttgtcttc aatgcactca ttttacctca gtggtggtta ctccatccca |
| 24361 | ttctccactg ctggctactc tccttgaact tttaatgtcg aagtgttcag ggctcagccc |
| 24421 | actgtcctct attatttttc atttctattc actctcttag tgatttaacc cagtcccatg |
| 24481 | gttctagata tcttcatatc tgtatcttta gtccaaatct gtcttcttaa ctttaaaata |
| 24541 | taactagctg cotttgtcat ttccactcag atggctaaca gacacttcaa atttaaacat |
| 24601 | tcccagttga aatttttatc tctccgtttc tgatcaaatt tgctctagct ctacctatag |
| 24661 | ccttccctaa ctcagtcagt ggcaacaaat tgctccgagt tggtcaagtc aaaattcatg |
| 24721 | gtatcatcct tgacttcttt ctttcctacc ctacattcag actcatcagg gaatccaatc |
| 24781 | ctcttggctg tgtcttcaaa aatataacca gagtctaacc acttgctaca tctctgacct |
| 24841 | gagccaccat cgtcttccac ttggatcagg gcagcgttct gtttaccagt ctcactggtt |
| 24901 | cttatcttca cctccctaga gtgtattctc aactgagcag aaagaagaat ccttttagga |
| 24961 | tgtaagtcac attatattcc tctcctgtta tatgtaaggc cctagaatag ttccctactt |
| 25021 | cacaaaaagt aaaaaccaaa atctctatca agtaatagta gtcattgccc tggcagtgtt |
| 25081 | tcaattaaaa gagtttaacg actggttcat tttatagagg tgtggacagt gtcaaggaaa |
| 25141 | cagaaaagaa caaaagaaca tgaagcatcc aggtactatc aacagtggga agtcctaact |
| 25201 | accctagtcc tgcaggggca aggtgagcga atagtgctat cagccctgga aattgctggg |
| 25261 | cactgtagga gaggggctgt caatagaagc tctaactgta gagtgcacac acttctctca |
| 25321 | ctatggtgag gcaccataaa tgtggtgagg cagagggaga gtgaggagaa tgaatatcca |
| 25381 | acctattttt tcttcacatc ccagccaaat acttggcatt gttagtcttt tttattttag |
| 25441 | ctattgtggt ggtgatgtaa tagcatccca ttgtggtttt attttgcctt ttcatggaaa |
| 25501 | ctgatgaagt tggacatctt ttgacaagct tgttgcccat ttggatatcc tcttttataa |
| 25561 | aatgcctgtt taagtgtttt gttcattttt ctttttggtt gttttttttt tttttttttt |
| 25621 | ttagatttct acaattcctt tacatgattt ggttgccagg ctttttaaag ctgtgtgtat |
| 25681 | tgcagatatc ttcttccatc tcttactggt cttttttctt tctttctttc ttttttttta |
| 25741 | ctggccagat ttctttaatt ttaatgtaag gtgatttttt cttgtttttc ctctatgatt |
| 25801 | aatatttttt atgtcttgtt taagaaacat ttgtctatcc aatgttatga aaatagtctt |
| 25861 | gtagaagctt tattgctttt tcactttcag atttatattt acgatcaacc tggaattgat |
| 25921 | atttgtgtag tggggagggt agaaatcaag actagttatt tttaatttgg ataactaggt |
| 25981 | aacctagcat atttctctgc tgctctgtag cattaccctt gtaaataaat catgtctcta |
| 26041 | tacggtttct ttctggactc tgttttacga ttgtatgctc tctggcatta gtatcacact |
| 26101 | gcctgaaatt ctgaagttcc atagtatctt ttcttttcct tgtttttttt tttttttttg |
| 26161 | agacagagtg tcattctgtc acacaggctg aagtgcaata gtgcaataat ggctcaccac |
| 26221 | agccacaatc tctcagggtc cagcagtcct cccacctcag tctcctgagt acctgggact |
| 26281 | acaggtactc accactacac ttggctaatt ttbitgttct tctgtagaga taaggcttca |
| 26341 | ctatgttgcc cagactggcc ttgaactcct gggttcaatt gatcctcctg catcagcctc |
| 26401 | tgaaagtgct gggattatag ctgtgagcca ccatgcacag cttcattgta ccttaatatt |
| 26461 | tgacagaaat acctacagtt tgttctttaa tatcaccttg aatatagata ttgagattcc |
| 26521 | attgaatcta aaaattgatt tggggaaaac tgatatatta gcaataatgg gctttctatt |
| 26581 | ttggccttat agcatttatc ctattatata atcattttta agtttttctt tttgtgtaga |
| 26641 | gaccttgcat atcttccttt toggtaccta ggtatgagac attttggaag ctatggcaaa |
| 26701 | ggatatttgt ttttgcattt cattgtcttt ttttttctgt gtatagtatt ctaggttggc |
| 26761 | atttattttc tatattctaa agattttttt ccatattacc ctctgttttt tattacatcc |
| 26821 | actatcaatc ttattgttgc ttotttgaac aagaaatgat tttcactgtt agcatttaaa |
| 26881 | attctgtctt tggggccggg cacggtggct cactcctgta atcccagcac tttgggaggc |
| 26941 | caaggcaggg ggatcacgag gtcaggagat tgagaccatc ctggctaaca cggtgaaacc |
| 27001 | ccgtctctac taaaaataca aaaattagcc gggtgtggtg gojggcacct gtagtccagc |
| 27061 | tactojggag gctgaggcag gagaatggcg tgaacccagg aggcggaggt tacagtgagc |
| 27121 | cgagatcacg ccactgcact ccagcctggg cgacagagcg agactctgcc tcaaaaaaaa |
| 27181 | aaaaaaaaaa aaaaattctg tctttggatt tcagtagttc tattatggtg tagtatttta |
| 27241 | gatgtagctt tcttbitgtt taatctgttt gatgatcata gcaatttttg aatctacagc |
| 27301 | ttgatgcttt ttgttatctt ggaaaatcct agaaaactac ttctttaaat atgccttttt |
| 27361 | ctttatagtc tctatcctcc agttaaatat atattaaaca tttgcagtat ctgctctata |
| 27421 | tctctatgtc acttttccaa tcatctatgt ctattatgct tttttttttc tagttttctc |
| 27481 | tggatgtttt ctactgatct gtcttccagt tcactaatcc tttctttagc tctaatatta |
| 27541 | ttaattccat atattgagac ctattttcag ttattacatt ttacagttct caaatttctg |
| 27601 | ttttgtaaaa taaattctat tctttgctca aattcttcat cttcctttct gttttctcaa |
| 27661 | acatactagt tgtagtactt taacaatacc tatttctttt ttttgaggca gagtcttgct |
| 27721 | atgttgctga aggtggtctt gaatttctgg gctcaagaga tcctcccagt tcagcttcct |
| 27781 | gagtatacaa tacctatttt aatatcaagc acaactgaag tttcattttt attgtctatt |
| 27841 | ttttctattt ttttgttagt cttatttgtg gatatttttc ttatttgctg aaaacttttg |
| 27901 | attgaatgtt tgatgtttta tttaaaaatt ataactgtct agactatgca gttttgcctt |
| 27961 | tctagagagg attcagtgta tcctctggta atcagccaaa aggcagacag atcactccag |
| 28021 | ttccattaga atatgacatg gctagaagct tttctgaagc ctgbitgtat cagtctggta |
| 28081 | tggcctaccc actttccttg ggagttgtct ttcaggatac ccattcagag cctatgaatt |
| 28141 | tatgagattt tttttctttg gtgggttctg aacttcaatt tttgtaacct ctgcactatg |
| 28201 | agactgttgc atacattctt gtgcttttca gaagatattt gcttggcttc tttgcttctt |
| 28261 | accatggata atttgaaaac ttagtgcctc aaaaggaaaa ccttaagtat cagggaacct |
| 28321 | catttctctc ttgttttgtg taccctctgt ccgtgatctt agcccctcaa atctgactgc |
| 28381 | ctttaaaggc ccaaacttta atgttttgtc tctccagctc tgtgaaaatg ctggcagtct |
| 28441 | cacttttgtt aactctgttg ttttctgcct ggattttcag cctcttacct atccaaggac |
| 28501 | tatcatatta tattacccaa gggaaaatgt tgctctcata atgtctgctc acctctctta |
| 28561 | ctttatgttt ctgcccagga tcttggcaac tgaaagcttg gctgtctcta tggttatatc |
| 28621 | ttcaaatagg tgtttgttgc ttttcgtttc atctcatatt ttctagttgt tctcagtggg |
| 28681 | aacattgttt tgtcacaagc tattccatca tggtcagaag tggaagttgc taagtcaact |
| 28741 | accttgataa ttattgtgcc gttagtatgg cttagtgact aagggaacac atttttgttt |
| 28801 | ggataggcct ttggtttaaa ttcctcttac tatgtttgag ataccgttcc attgaaaact |
| 28861 | tagttttatc acctgtaatc agaaaaaaaa agaccttttt ttagaggact cttaggagga |
| 28921 | ttaattggaa aatattcacc taaagtactg attacaataa ccatcacata gtaaatttag |
| 28981 | taaatatgtc tcaaatgata cagaaacctt atggaacctg taaggttaaa tcatgcattt |
| 29041 | ctgaaagtag taataatatt tactttactt ccagtgggtt aatatgaacc aaattatttg |
| 29101 | aagatatttc tattaaatat tgattacgtg aaagaatata acatatactt aagatctata |
| 29161 | acaataaatg aatgccagtg taccgctact caagttaaaa ataaaatact accagtccca |
| 29221 | ttgagtttta tgtatatgta ttccttataa atatttcatt gagtttttgc ttttgaaatt |
| 29281 | tatgtaattg gaatgatgtt ctatgcacct ctttgcaact tgcattatta taacttgctt |
| 29341 | tatcttaata tcatctctgt gattcattca tgttaactat agtttttttt cattttctct |
| 29401 | tattttcaat gtggtgagat tatgttgtat aatatctccc aaatatgcat ttattattaa |
| 29461 | tagaaatggc gtgctttttg atctttcact tttggtcctt tttccttgga attgtatttg |
| 29521 | ggaattagtt gaagatgaat ttctctagag cagctttttg tttgcttgtg ctccgcacct |
| 29581 | gtggacacta taaatagaga ccactttcaa aggtattttt ggcttaagtc tgctcgaacc |
| 29641 | acacagatag tgttacttca gaccacaact aggatgagag ctttctcctg attatatccc |
| 29701 | aaggaggaga tttctttctt ccttttaccc agagcaaatg ttgagacagg caaatttcct |
| 29761 | tgcagtctcc ttctgtagat cagagttatt tttctagttt gcccttatat tgagaggaca |
| 29821 | ctatagattt gttctagctt aacttaggga actactatta gactctttaa gagagctctg |
| 29881 | ggctctgttg cctgttaaac tgtcaagttc aaagcccagg ttcaccagct ggagcaactg |
| 29941 | ttccagggtg aatatgactt agtgttgaat actctctgaa ttatttcact ttatttttgc |
| 30001 | acccctagga atactttacc ttcttaatag atctttatat gcagacacac atatgtttaa |
| 30061 | acatatggaa gtacatatgt gtgcatgtat gtagataaat agatagatgc actttttcag |
| 30121 | aaagagtctt ctaccgaact gttagaaaca gaaatgtctt aagtctgatt tgtgctgctg |
| 30181 | tgacagaata cctaaaactg ggtacatgat gaaaaataga aatctagttt tcacagttct |
| 30241 | agagattgaa acgttcaaga tcaagttgct ggcaggtcag gagcctggtg agggccccgt |
| 30301 | ctctgcttct aaggtggcgc tttgaaggct gcgtcctctg gaggggagga atgttgctcc |
| 30361 | tcacatggca gacggacaga agagcaaaga gatagaaccc atgatacatg aaagcccttt |
| 30421 | tgtaaaggca ttaaacccac caatctgggc aaaggcttca tgacctaatc acctcttaaa |
| 30481 | ggccccacct ccccccatat tcttacattg gcaattagaa ttcaacatga gttttggagg |
| 30541 | ggacattcaa actataccaa gaaataactg attcagtttt tataagaaat tttagaactg |
| 30601 | tccctaagac ctggtgagtt tcaggcctca tgttctgagc ctacatctga catcagctac |
| 30661 | catagtatcc gctgtgcagg cagagcctct tcagtcatag gtatggggtg aaacagattt |
| 30721 | ccacctcttc cctgagcttc gctggcacag aataaatgca ttctaaagaa aactgatacg |
| 30781 | tcagagacat ctgagcttac tcttaattgc tttcttacct ttctctgtct tgggccagat |
| 30841 | tttcttccag gctgatggct tcttccaact ctgaattcta gcactctctg ctgtttcatt |
| 30901 | ttgtattcca tgccttcgtc tcttctctga tcccttccat ctgcctgaaa gcacttcctt |
| 30961 | tttattattt ctgtgtccta taataataaa gttaacaaaa gtgttaagta tatgtttatt |
| 31021 | tttccaaggt tacataggca catgtataat gtagggaaac tggctgacaa tcagagacag |
| 31081 | aagacacata cacgtgggcc cacacacatt cgttcactgt tgtccttaat gcagtattca |
| 31141 | acattacaaa ttactgtaat cttaataaaa ctgcgacatt gttttccttg tttatttagg |
| 31201 | gagcaccttt aaaaatcatg tatttgctga tttattcatt cactattgtc cttaatgtag |
| 31261 | tattcaacat tccaaattcc tgtaatctta ataaaactgt gacatagttt ccttgtttct |
| 31321 | ttatttaggg agcatctata aaactcatta tttactcctc agaaaacttg catgggcata |
| 31381 | tagttatgaa ccagtttgaa tattatgatt ttatattcac aatattttta tacttatgat |
| 31441 | cctaggctaa ggatatttat attagaatgt tcgctgagga cttaaataag ttctcactga |
| 31501 | tacttacact gaatattatc tcaatgtact tgagagaaaa ggtattgcaa gtaaaggtaa |
| 31561 | atatatgagt acatcaattg tataatttaa ttttctcaca attttaggac aattttgtat |
| 31621 | aggatctata atcatatact tttttaaata ttcacttgct tttattaaat caatcacaag |
| 31681 | aaaatacaga acgttttccc cttccacttc tgaagtggag gaaatcactt caaattcatg |
| 31741 | ggatctttga actttgccca ttgggccaga cttgcagtat cataaacttc tttttcctac |
| 31801 | ctatgtatga ttttgccatg tgcgaaacaa tcattgctag taccacgagt gagtgatgat |
| 31861 | aagcttagaa tacatccagc taagatcagt gccatttgaa gctccctcaa tatcgcattt |
| 31921 | cttcggttct ttaaaaaaaa ttcattgttt tggagtcagc atattattaa tgaatggagt |
| 31981 | atttgagggg aataaatatg actttggcaa tatcacatgc atgggaaaaa ttggctagag |
| 32041 | aaagggctaa gtctttcatt ctgtgtttta gagaacagct ttagctcgta tgaaaaaatt |
| 32101 | ctggcataga tatgtgaggg ggagtgttgg ataagaaaag gaaagagaaa aagcttgctg |
| 32161 | tggagtggaa ttctcatcac atttctttct tacctactac tctggggaaa cttcagttac |
| 32221 | agggctgata aatatttatt aagaatatat agacagtata acctaaaaac attggaagat |
| 32281 | attttagcga ggcttgagtc agattaaatc taccaaccgc taaaagatgg ctttgcctag |
| 32341 | agaaattctt ttttctttgg gagcagctat tagggaaaag cctattgcct aatatcttaa |
| 32401 | ttgaaaccta tgcataaaat tcaattaaaa atggcctata ttttgaattg tttttcaagt |
| 32461 | tacagaagaa ctttaagatc tttgtaggta atttctaaaa agcataaata tcttaaaata |
| 32521 | tatatctgta ctatcaccat taaaacttac cattctttgc tctatatttt ccaaagctat |
| 32581 | attcagagaa aataaaacct attatttcat agcctgcttt aaaaatatct cttaaaatac |
| 32641 | gctatttttc atctcctgat ctatccctca agaatatgat tttaaaactg cattacctca |
| 32701 | tttcgactta tttgttttat gtcctccttg ttattaacta tttttaattt tttctgtgta |
| 32761 | aaaacactta tttctttaca tattttctct aaatttggaa gctatgcctt cgtcaatgat |
| 32821 | accatgttaa cattttaaat ttttaaaaat attttaatat attttattca aacagtccca |
| 32881 | ataattaaat ttatttcact ggcaaatgcg atataaacca attgctgcat ctctcctctc |
| 32941 | caaaaagcat gtcttgttct agtgatgtat ttggagatag aagacttaaa ttattttatt |
| 33001 | tttttttact ttttaaaaat gagaaatgaa aactojgcta tatgcatggt gtacactgtg |
| 33061 | acgttttgat tgaagtacat tgtggaatga ttaaatcaaa ctgattagca cattatcatc |
| 33121 | tcacatactt atcatttttt gtgatgagaa tgtttgaaat ctactctatt attatttacc |
| 33181 | atagtcacca tgagtatatt acatcttcag aaattatccc ttctgtctaa ttgaaactgt |
| 33241 | accctgtaca caacatctcc ccgttgacca ccccaccagt ccccgagccc ctggtatcca |
| 33301 | ccattctact ctctgcttct atcatttgat ttttttaaat ttctacattt aagcgtgatc |
| 33361 | atgcatattt gtctttttgt gcctggctta tattgctaag cataatgccc tttagtttta |
| 33421 | ttcatctttt tgcaaatggc cggatttcct tcttttttat ggttgaacaa agtacctttt |
| 33481 | ttccccccac tgtcccaaat atatgggctc aacccccaaa ttggcaggtc taagaagtct |
| 33541 | ttccaaaggg tagtctatta catttaattt ctaaacataa gttgaagaaa agaaaattta |
| 33601 | tcctgacagt tccaaagttc aataaaaagt atagtttttg agtttttact tcagtaaggt |
| 33661 | attattttag tttottagct tttcagaaag totcagtott ttcaatggga aactctctta |
| 33721 | gggcatttaa aatataattt gctaacaatg aacttttaga tgagtaatcg tggctgctat |
| 33781 | cactggccct ggatggcagt gggatgagct tgcaatgcca aggatacagc tgagcaaatg |
| 33841 | ccactggaag ctaaataaca tgaaagaaac cagggttttg ttgtttacaa tggccatgtg |
| 33901 | atggtatgag tacaggaaag ggctagaacc tgggcatctg aggagaaaaa gcattgacag |
| 33961 | catggatgta aatgacgact ggtatttgaa tgcaatgtgg ggccagaggt agacatcatg |
| 34021 | gaaaagtatc agactttgca tacaggacac aaactaaagt aatcactagg gttagaagag |
| 34081 | gagcccactt tctggagata ggttactcag agcaagaaat gcagcaagtc agaaagctgt |
| 34141 | cgatttggga agctacttat taatgtacct tacatcctcc ctgacaacaa tcgagattga |
| 34201 | taaccacatt taattcagtt ctagtcagta agacggaggt tttctagcac cagctgccaa |
| 34261 | gattaaaaaa tgtgcatgtc ctagctctgg gaagtgtctg gtatgattga tgcttggtac |
| 34321 | ttgttcatct aaatatgaat ttactactgg gaaagaaatt aaacttctag atattcattt |
| 34381 | ggtagatcgg tgaggtgact gaagtgtggc aaacctcaca atgattatga ataaacttta |
| 34441 | ttaagaaaaa ctgaggcaat taacttattt gttcttcaaa actgaggaaa tatttgtcct |
| 34501 | cacatatatg atagaatatt gtttgctgct ggtaaccaat ctttgctttc ttctactaac |
| 34561 | aacttgtttg tcaatatgtc ttatttctca ctctaataag actatgtcta aataacttct |
| 34621 | ctcctaaggg ttaaaaatac aaagaatttt cacacataac caagtaataa agaatgtata |
| 34681 | ttattgaatg aatgagtaat ttacaaaaga aagtgttgag aagtagaata cataatttca |
| 34741 | gatttatgaa atatgaaatt tcagattcag gatctgtcca gtggagttgt gtttttactt |
| 34801 | ttatggggat taagtcaaag gcagtatatt aaatataatg gaataatatt cccccaaaac |
| 34861 | atggtaaatc tatactttcc ctgaatataa attttccttt aggacaccaa atctttaatt |
| 34921 | aggttaatta taatttcagg tagattaaag aggactattt ctctttaaac ataaggataa |
| 34981 | ttatatgaga attaaatctg tgagaaagaa ttggaacctg aaaaagacaa tggagaaaac |
| 35041 | tagcagacgg tatgttcaac tagaagaaag agaatttaaa agttaaaccc aaggtggtga |
| 35101 | ataatcacaa aaattgttaa tgaagagcac tttttttttt ttgctgatgt tctttaaagt |
| 35161 | tttttagccc tggtaataca tatttagaat tattttagaa tttgattatc tatgactgta |
| 35221 | ccaatattat tgtgttagca gtatgagtct tgctttctat tattgaaatc gttgttaaca |
| 35281 | gatcaaatta cccattatag ttttatgctc tgtgcataca gtggcatgac ttagattaga |
| 35341 | agaggtggat aabittgttc tcatattgat attataagct tttatcacag atctagggac |
| 35401 | ctttgtcttc taattcttgt gtgtttatta ttgcttattc cattcaattg gcatgtaatt |
| 35461 | gaaatggaat tgaagacaag tacacatgtg tgttccttta tggcccctaa cattcactgt |
| 35521 | ttagttatat aggtctagat tatgaatggt gcctagaaaa aaaagtatga ataaggaata |
| 35581 | attaataatg aatacttatt tccatcacct gttaacatac tctgtccatt tattcctcat |
| 35641 | tttaccgtct acatttaagt tctcttttct agccaccaag cattattgtc tagctaccat |
| 35701 | ctactgagtg tttaatatat gtccagaatt gagcagagtg ctttgtatgt aattcatata |
| 35761 | accaatctag caaacatcct tgatttttca ttttacctag ttgcctaaac cttccagcaa |
| 35821 | tctattagga agtcaaaaag cttctcttcc tctgaaatat attccaaatc catccatttc |
| 35881 | acctgttatt cttctaggct aagcbgtcat catcctttgc tctggtgatt gcaatagaac |
| 35941 | ctaattgtcc tatctttttc ttgotttaca ccottaaaat ctaatctact tcttgaaatc |
| 36001 | agggtgtgtt tcattgtaaa tgagataata tcatttctac atcttaacct agcagtgact |
| 36061 | tctttccatc atatctggac taaaatccaa actctgtatg gatgataaga cctatcagaa |
| 36121 | ttggagtacg tttttttcat ccccatcaca tgccatgccc gttcttttca aaaggctacg |
| 36181 | gctctttctt gtctaacatg taagcacatt cttttcaaag agcctggaat gttcttccct |
| 36241 | agatgggcat gactggctct tttttgtgga ggtcccagct accaattcct taagggaatc |
| 36301 | tacccttcct tatctgttct aaagtagcca cccagtttct atcatgaccc tcagtttatt |
| 36361 | gtcatcaaca cttcattttc ttcatttatt gtttattcct taattgattc tacttcttta |
| 36421 | gtagaaattc cagaagcttt aaagtgtttt gttcatagct gaatgcccag aacctagact |
| 36481 | aatttttgac attcaaaaaa tagcttataa gcatttagta cattttatta aataagaaaa |
| 36541 | tagtatctcc tctaatctca gaatattata aatttatatt gttattatca cttaatagat |
| 36601 | ttaaaaagtc atgaatgtgg gagataagcc cagggttatc cagttatttc agtggcagag |
| 36661 | tcagaatttg agctcatgtg tttcagacct tagaacttta cctatagtct ttgatgtcta |
| 36721 | catcacatta tgctccctcc catgtaccaa attattctct taacttacta tatcaactaa |
| 36781 | tatatctata aaataatctc tggcatagga atatagcatt catataataa atcattactg |
| 36841 | taatgcgttg acaatgcatt acagtaaata atgcattact taacctgata cattattcag |
| 36901 | ctgtttagat gagtcatcaa cctaaaaata tctgactggc tgtacagtaa tgtggtaata |
| 36961 | cattcatcat agagaaatta acgtatgggt tgaatattca tagtgatatg aatacaaata |
| 37021 | tgbitatcag taggattatg gatgtatgat agtttagtat ttacctagca tgtcaatgga |
| 37081 | tgataacaat tatctgcaaa tatttctgag tgttttaatt tgggtttccc tagaagtaga |
| 37141 | ctttgagaca gggaattgag gcaaggagtc tatctgggag gcaatcccag ggcactttgg |
| 37201 | caggaaaatg gtaaaatgag acaggatggg agaggaccca ataaatggta tgttatcaag |
| 37261 | cgagctatcg ttatgggtat ctggagttta attctacagg ggactctatg aaacagtgta |
| 37321 | gaacacacaa ctcagactta tcctgaccaa gatgcaaggg agcbjggtat ttatacaaaa |
| 37381 | agtcttgtta aactttggct tagggctgct ctggggagca taattacttg tctcctgtct |
| 37441 | gctgtaagtg ggcagcatgg gttctggcag taagagcaaa aactcaggga ataaagtgga |
| 37501 | gatgataaca gttgaaagtt ggactggtat ctactgaaat actgagacca tggacagctc |
| 37561 | gcaggctatt aacaaggtct tctaaactac tcatatatgc atggtaatct gcatgcattt |
| 37621 | ctgaatgtat acatctatac gaattggaac caacagatga aagtaactac atgaaaactg |
| 37681 | tgaaatatag aaattactta cctgtaatag gacagataga atatacagac aatgctctta |
| 37741 | agttcatggg tttctatttg aatgtatatg ttggtgcaga ccctgtgaca atctgtattt |
| 37801 | tcaggaattg taggtatatt tgagaaagtt atatttttat gtaatatata acagttctct |
| 37861 | ttaggactta tcagtggata tcctaagaaa cttttaaata ttttcctatg tttattcatc |
| 37921 | ccacttcaca ctggtataga tagatagttt tttgataact caaatctacc tgtggtaagc |
| 37981 | agaataatgg cccccaaaga tgtccacaac ctactccctg gaacctgtgg acttgttacc |
| 38041 | tacatggcag aaggggtttt gcaggtgtgg ttaaattaag gatcttgaga tggaaagatt |
| 38101 | atgatcctgg attatctaca tggttccagt gtctttacag gctctttata agtgaaagag |
| 38161 | agaggatgtg gagtcagatt cagaatggtg tgatgtgaaa aagactcatc tggtcattga |
| 38221 | tggctttgaa tgtggaagga agccatgagc caaggagagc atggagcttc tagaagctag |
| 38281 | aaaagggaag aaagtgtatt cttctccaga gccccttaca ggctttgcca gacaccttga |
| 38341 | tcgtagccca gtgaaaacca ttttggacat ttcacctgca aagctgtaaa aaaaaatgca |
| 38401 | tttctaatgt tttaagcaac taaacttgta gtactttaca ccagcagcaa tgagaaacta |
| 38461 | atgagctacc taataaacac agaggtaaag tcacctcaag taaaaaaaaa aaaaacaagt |
| 38521 | taagacctat gttttaaagg tcatagcagt atttccactg tccgtatgtc actgcagtga |
| 38581 | taaatgctgt gttttgacca ttgaaagaat tojaaattcg agtaagcaaa actaagttgg |
| 38641 | aattctgcct aattactctc tatttaacct cagataactt ttaatttctc tttgctttat |
| 38701 | gttcatcatc tggaatatgg ttacagtaat aatattctga cttccctcat gagttcattg |
| 38761 | taagtattaa tttgaataac ctaggcgaaa ggacgttgta gtctgcaaca tagtacatgc |
| 38821 | aattttattt ggctttgtta gcaaataata gtgctaaaag aaatcagata tccaaaagac |
| 38881 | ataaattaaa ccacataatc ctagtcaatt tgtagatttc tgagtatttc tgtatcaaac |
| 38941 | tggcaagtat atgctatatt tatgatgagt aagtcataac agttaaaaga ttgactatta |
| 39001 | attgagagta aaaattgtcc ttgttattgt cttatgcatt ttaatttgca ttttctgtgc |
| 39061 | cttaaataga aatattttaa aacaattcat gtatgcagga ataaaagcac tttagtggac |
| 39121 | tattaaatgg taaacttctc cacatcattc atgagggagg agttataaaa ctgttctaac |
| 39181 | tctcaatttc ctaaaacttt ggtaaaataa tattctatct catattctcc cttctgtatg |
| 39241 | tctttatgtt tgcaaagatt ctttggggca cagaaataaa caaatcatcc ctatgaaagg |
| 39301 | gtcatcgtag ctgccagtga ccaaaagagc tgcctgctga gcagtttcat actgttactc |
| 39361 | cagctgccat ggactcatgc gtgaggaatt cacacagcta atcaattcca taaggtgcag |
| 39421 | ttgctggctt ctctcttctt aagcctgtac ataaggaatt ggcacttgag tgtttcatcc |
| 39481 | tgtggactct acatcattcc ttcacaccct ctgccatttc ctggccctcc ccaaaataga |
| 39541 | cccaaagcaa aacaataata aacaatttgc tgtttgattt ttagtcagca ctaccattca |
| 39601 | ccagccctta agaatgccac agcagctgcc tgtcactgac bitttcagca ggatcaggct |
| 39661 | agaggtagga gaggcggggc aaaatttctc ctccttgtag gttttagtta attgctgtca |
| 39721 | atgacaacta gcagttgtgc acctgtttcc atgcatttag tttctaccct ccatcaataa |
| 39781 | attctaacaa ttatttattg aacatattct atagttttgg tccttaaaat aacatctgat |
| 39841 | tataggtact gttgttatca aatgaggaag cagacttcac aaatagcaac tgggccacaa |
| 39901 | atcttgctac caagtgtcct agccagtggt cttggaagtc taggctcttt ccctatagcc |
| 39961 | cccctgattt tattagccaa gttctaattc taacatggga aagtcctatt tcttagttat |
| 40021 | gtgtgatgta acttagattt gtgtgtaagt tattatttct tagttttgtg tgatataact |
| 40081 | tacattatca tgtacatgtg cacatttaca tcttacatgg gaaattttaa tcagaggatg |
| 40141 | tcttcatctt tgcgtcacta ctgaattgac taaagactca ctcaaagaca tttccgtttt |
| 40201 | aatagtgaag agaaggaagt gattctcttt gcatcttatc ccttcatcct acaggcattg |
| 40261 | tgctcctact gaacactgaa gagtagtttt gttggcagta accttagaac gtttggagtc |
| 40321 | aaaagttgtt gtctctatta cactcagatt gtagaaagtc agcaggccat tcggagtgcc |
| 40381 | aatatgagct atgtaaaagt catctcaaga ctggaggaag agtgactctt gaggccttta |
| 40441 | ttttccatat aaattgtgcc ctctgtttcc tagcaccttt caggatccgg ggcagcctgt |
| 40501 | ctgccatttt ccacttgttt agggctttaa aatcgtaaag aacttcatta actccatgaa |
| 40561 | ggccttcaat ttaatattga ggatactgga gtatttatca cagaaaagat acagctcaaa |
| 40621 | aattttaacg caatttcatg gaaaattaaa aaaataacta ttacattttg gtaagacttg |
| 40681 | tatactgaat ttttcagaat gtaagctaaa ttgggtttaa tcttatattt tataaaaaca |
| 40741 | aagaaatatg aaatttatcc atttattttt tttttcttta tgacatattc caaatataca |
| 40801 | gaggaggaca gagacctaaa agcctcaggg acccatggtg gggctctatc aaattctttc |
| 40861 | attttgcctt atgtttccag atattgttat gagaaaaaaa cttacaggta aattgaagac |
| 40921 | ccccatataa gccttcagaa atctcattcc ccttcctcca ccaagttaac caatattctg |
| 40981 | aatttagtat ttatattttt atgctttttt tttacgttta cacatatcat tgatctatcc |
| 41041 | aactttttgt aaattatgtt gtgctgaaca tacattcttc aacatgttat tttctgtttt |
| 41101 | bitttatgtt ttattttagg gattaagcct tgttgatgcc tatagcttta atatatatgt |
| 41161 | agaaatctgt tgtatgaccc atgggttagc aattctctgg taaaggatat tccattttct |
| 41221 | tcaaacattt tgctgctgca attagtactg tagtgaacat tttgatgttt ccttataaaa |
| 41281 | agaatggtgg tctttttagt ttgtaggaat tctttgatca ttctattaat cctttgtcaa |
| 41341 | ttatgtggtt gaaatgcctt gaattttcaa atttatttgt atgaaaatac tgacagtagt |
| 41401 | cccttatgat ttttgccatc cattctataa agagctattc gcgtttcaat tctaaaattc |
| 41461 | tttgtaagtt ttctctttca ttcttggaag actgttbitt tactctttag tctcttcaaa |
| 41521 | gaattttgtt tggttttttg gctctctcta tttctgtgtt tatatttcac tattttatac |
| 41581 | tgttgctttg attaaatcct tccttttaga ttgtttaagt acattttcat tctttttcta |
| 41641 | acttcttgag ttgaattacc ttaaatttat tattctcagt cttatagttt ttaataaatg |
| 41701 | tatttcattt attaaataaa tgtatccatt tctcttttaa cttcttccca caagtttgga |
| 41761 | aaagtattcc ttttattgta aagttctggt tctctttctt ttttttcttt ttgtcctatt |
| 41821 | gggatttcct gttataaccc atgggctact gaagttttca aacgtataga aattttttgc |
| 41881 | agaaagtttt tgcagtatct attaattaac gatttctatt gtaacatcat ggtaaaaaaa |
| 41941 | cattggttct gtaagatacc aattctttga aattgtttgt gatcttttgg aacccagctg |
| 42001 | gtggtcagtt ttcataaata gtttttgtgt atctgaaaaa gatgtcaatc cagacaggat |
| 42061 | tttatatgta tacctatgag aagaaacttg ttaaatttat gttagatctt ttctatttta |
| 42121 | ctaaattcct gcttcccctg aagggtttag gtatgcaagt cctttagaaa tctaaaggta |
| 42181 | gtaatgttag gtttataatt gaaatggctt ctcagtggac tctacgtctt ttactgtatt |
| 42241 | gtgaaccctt tgatgcattg ttttacattt ttttcttact ggcattagta ttactacata |
| 42301 | tataaatttt tttcaagtta gtatctatat agtatatttt cttcagtctc tattttaaac |
| 42361 | tttcaaaaca aatatagttt tgttttaggt tattcttgta atcatcatgt agttttataa |
| 42421 | aaatctaatt tgattatcac taatatgtga gtttgataaa tccacatagt cataattact |
| 42481 | gatatgtcac aacacagtaa gggcbgtggt cttcatttat ctgaacaact gctttacatt |
| 42541 | tagtttcaga gatcggagta ataatgaacc tttctgcatg tacccagtgt tatgactcct |
| 42601 | gtaaggatta atttaaaaat gttcaatatt tattttcctt ttggattttg ctgaaaattt |
| 42661 | aattttagaa ataaaaaaaa tctctagttc tttcaaattt tccatgaatt gaagattgtt |
| 42721 | taccgatcta tttttttaat actgcatttt ggcataattt tttaataaat gtttacactg |
| 42781 | taattttcag taattcagtg taaagctaac aggttttttt caaagatgaa gagacacaat |
| 42841 | atgttataga atatagtaca tggtgggaca gttaagtagg ggttctggag tttatttttg |
| 42901 | cttctgctac ttaactatgt tattacaaag aaaaaaatca tgtaagagac tccgccaaat |
| 42961 | tctcttctgt ataacaagag ggagtgacat actatctcaa tttcagtttt catatttaat |
| 43021 | gactcacaat tggatttttg gtatatattt gaccactttc taagatttta ttttttatgt |
| 43081 | tcttgaaaat tttatattct ataaatattt ttgtatacat atgtacctca cattcaaggt |
| 43141 | aagaaattct taagtaattg aactttgtgg ctataattaa atatatacac taatagatga |
| 43201 | cagaataaag aaaatatttt tattctgaga gcctaatttt aggtaatgaa aatcatttta |
| 43261 | tgggtaaata aagtacacat tttatagtgt gctaaaaaat acacaaaatt tgacatttaa |
| 43321 | aacaataaaa aatattaaat aacttggaat gaaaggagca tatcgaagta tttgtgaata |
| 43381 | aataatctca aattgatttt ttccaacact ataacatctc ttttctgttt aaaagaagtt |
| 43441 | taatagttgg ttcaatcttt tctttgagtt tgttttttat attttccaag cttttaattg |
| 43501 | tcagtggagg gagaggactt gagtgttttt aaggtttcca tcaacatcca ttctcctgta |
| 43561 | ttcattgtta ggccaaaagg actcaacatc tttgagtctt ccctatttga gaccaagtct |
| 43621 | atactcagct tctaatttca gctctagctt ccattcactc tcagtctttg ttagcattat |
| 43681 | ctaagtttag gttgagatta ttgcattatt gacttgcatt ataaacttct tctttcaaga |
| 43741 | ataatatgac cttttaatct tgttatatat gtttacccat catttacata aatttttatt |
| 43801 | ttatattcca tatggtttta ttccaacagt cttttgtagt taaatatttt atactcattc |
| 43861 | atcataaatc ttcaagtaat ttttagaaga attttgtaaa agtatatgac ttttttaaat |
| 43921 | tttgtgggta gactatatca ataaaatatt taaatatatt cagaaacttc ttttaaagtt |
| 43981 | catatcactt tactttggta aagagtttta gcaaaagctt ataattaaaa tatgcgacag |
| 44041 | tgctcacaga gcatttctga ttactatcag aaataggggt ttattctgga aagttgtttt |
| 44101 | cctgaaagtc ttaattgtaa aaagtgcagt ttaataccta ttatattctt atgtgtatat |
| 44161 | taatgccaaa ttaagcaatc ttcctccctt tctttcttcc ttctttcctt tcctctttcc |
| 44221 | ttcttttctt cgtttctata tttatgctgt atacgtacta cttgaaatat ccagacacta |
| 44281 | ctatttgttt aattcacaag ccatatctgg tgatatggat attattgttc ttgtttcata |
| 44341 | gttgaagaaa ctgaaacttt aatgaggttg gataagagcc acaagtatag cagattttat |
| 44401 | agtgtataac cctgcaaagc tcaaatttgg attgatatct ctgtttattc tagaaccctc |
| 44461 | ctctgtgtca ttctatgtca gtttgctttg ttgtttatta tggtaaagta atcattcttc |
| 44521 | acttcaacag ttatctgtga gttgattcta aaacatagaa aaattttgtt ttaaaataaa |
| 44581 | ttgtatgtat gtoitcacct aataatgggc ctatgtgggg tccacacaaa accagtttgc |
| 44641 | tgcagtcttg gcatagtgta aaattcccaa agtttttata tacatattga atgcctcaag |
| 44701 | gtatatttta aaagcccttg ctaatcccaa aggaattgtt ctaaaaattt gtttaaatga |
| 44761 | aatttttata tattttaata tttaaatttt aaataatatt tttaaaaatc ttaggctgaa |
| 44821 | ctttcagttt gtaattagca aagtaattat tatttgcccc aagccagctt cctcctattt |
| 44881 | cctgaccctc atttgtcttc cctccttata agatgcttaa tgaaagagaa cttgattttt |
| 44941 | gttcatttgt ttttgagaca gggtctggct ctgttgccca ggatagagtg cagtggcaca |
| 45001 | attgtagctc actgcagcct tgaacctggg ctcaagggat cctcctgcct cagtgtcctg |
| 45061 | agtagcbjgg actacaagct catggctaat gagcctggct aatttttaaa ttttttgtag |
| 45121 | agatgggggt ctcgctgtat tgoccaggat ggtottgaac toctaggctc aagcagtcct |
| 45181 | ccagcttggc ctcccaaagt actgggattg caggtgtgag gaccacgccc agccttgaaa |
| 45241 | cttgattgtt atttacatgg tgcacttaag aagcctaatc ttaccttctg acccagggtt |
| 45301 | tgcagtcaca gtccttagag tttttgaata aagactgtgc tatttctttt ttttaaaagg |
| 45361 | gatttgtttt tatctaattt atttctgcct ttatgtttac tcattattaa ttattcaaaa |
| 45421 | taagattggg atcatcattt atgtatgaat aactcaaatt agtttattgt aatacatagt |
| 45481 | cctggtgaag aagaacatat tcagttcatg ccttcaattt gcatttatct tgattagtgt |
| 45541 | ttaagtgcat ctcatttccc tatgcttctg ttttcatagc ttatataact acactgttag |
| 45601 | attttatgca tgatgtaaca ggatacaatc ccatgatata tttccatgat gtcaaagctt |
| 45661 | tgaattatgt aaataggaaa catgaagcaa aagcatacta cttattcatt atgcattgct |
| 45721 | atgojgcaga cgcacagtca agtgatgcta tttaatctcc ctatctgcat attcttaaag |
| 45781 | tgcatatctc tgtgatatcg aagcactcaa gcaatgagta aatttctgca tctggttaat |
| 45841 | aaattcagca aacaggagtt catcaacatc tggcatctgc cttattttac tggctttata |
| 45901 | tgaataactg atggatggcg acctttgata aatttagtgg ttgcttggaa ttttagtagg |
| 45961 | ggtttgatat tgtttgtgtg ctattgtgaa tttaatatgg caatcaactt tctcttggtt |
| 46021 | gctttactca tactagaaac cacagcatca accaatcaaa aggcbgttta gcatttggtg |
| 46081 | ttatcacata taccctaatt tgcacaactt tccaaaatgt aagtaccaag agagaagaca |
| 46141 | catcaataaa atataggaat tataaggaag aggaagaggt ttttaaaagt atattattat |
| 46201 | tcagcaaata atggaaactg gtgctcacaa actactaaat taagagggct tttttccttt |
| 46261 | aaaaattaat gcaatgatag tgcagaacac caaataaaaa ctgcatagcc aaggctctgg |
| 46321 | aaggttgctg gtggctcctg gacatcagtg ggatttcaca tctttctctc tgacatatca |
| 46381 | tttgttcatt tccagagaat gcccagctct gtgttttctt aaaaataagt ttagaattct |
| 46441 | cataaaggtg gacattattg gcctagcatg agttaatttt ctctacccct ggacctggac |
| 46501 | caatcatata tataagcaga gagcagaaag gtagcacaga ctgaggcaag gtgaggataa |
| 46561 | ggtatttttt gbitgtcaca gagacacctt gcttgtctca tttttgaaaa tgatgaaaac |
| 46621 | aaatggtgac taataaatag aaaatgctct gtggtattat cttctagtac taatgttttc |
| 46681 | ttcactgtcc ctggcctggt atctttcttg gcatacctca caaattactt gacttctgtt |
| 46741 | ttatgttgac ttcactcctt tacattacat tattgggaag ccctaggtct tagaaaacta |
| 46801 | tttcttaaac tgaattaaaa ttttgccctt gtacactggt actggctctg tcctctcagt |
| 46861 | aatagaaaag aagtcttcca tatgtccaca gccattattt gcaaaaagat atctctttct |
| 46921 | tcaggcttat gtttcccatg ctacatctct cttgttctct gaaccacact ttatacagcc |
| 46981 | tgactttcca tattctcact gttgctgtca tccccatctg atcattcccc tctgtcttaa |
| 47041 | aatgtgacaa ttacaactgg acataatact ccatctgtga tcttctcagg acagacccaa |
| 47101 | gcaggtccca gtatagtgtt agcaggacat cacatttttg gcttcagatg aacctgtaaa |
| 47161 | tacctaaagt acctggactt ttcccatgaa ttgttggtag gccaggatgt gtgccccatt |
| 47221 | agttggggac actttctagt gacagttcct ggaactctat ctggctattt ctctagtcag |
| 47281 | gcagtgaagg cagataaatc ctoggttcct ggcaggttca ggtggaacac gcagcacagt |
| 47341 | gtgcgctgtt gaatgagtgt tcccgagatc taatcacatg gaatgcctga ttgtcaaatg |
| 47401 | atgaacaaat aattcaggag atggacctgg gagttgaaaa tttttttaac aaaaaagaaa |
| 47461 | ttacatatat gcatttttaa caaaagtttt ataatcattg atatagtgat ggtagcacca |
| 47521 | gtagatattc aggcaagtaa ttatcaactg gatgacttat aaaggaattg ataaagcctc |
| 47581 | ccaagggatt caagaaaagg gtaaagcttg gaagtcccta aggataagtg gctgagatgt |
| 47641 | ttgattcagg acttcctgtc cttgaaacct tggtcagtta ctagcacgta ggacagtctc |
| 47701 | tattgtggac cagtaaaaag ctggttgtct ggagaattca gacagaaaag gtaaggctgg |
| 47761 | gacccagtta tcaaggctgc actacaacta gggcttgtaa aagttagggg attttggaat |
| 47821 | tagcgtagaa ataatgtaat atggtgtaag taacacaggc tttgtaatta gtgcaaaaat |
| 47881 | cataagcata tgggcttgag tcagtcttct ctttcctact tactggctgc ataccttttg |
| 47941 | tcagctattt aacctctctc taagcctcag cttcttcatt tataaaacaa attaatatta |
| 48001 | tctacactga agtattatag gagagatgat aatgtatttt ttaaaaacta gcacagaccc |
| 48061 | tggtcaacaa taataaacat actaataatt gttactatta taataaacgt gttgatatct |
| 48121 | ttttcttcat caatgacttt ataaaaatat tgggatggag atgccaaaat ctcctctgct |
| 48181 | aaatgccatc tttcatccta aggctataca ggaacaggcc ttgcctactg aactttgtag |
| 48241 | ctataggcct actacattgc ctcccacttg aaccctoggt tataaacgga atggagagtt |
| 48301 | acttttccat tcagaaaaga accttgatat ttcagaagga aaagttagcc ctggacactt |
| 48361 | gagcatttct aagaaaatag gttttttttt tctttttaaa ttgtgtacct gctgccctga |
| 48421 | attctctaga tgttctgagc actctgtata ttcttactca tagcttagta ataaattatg |
| 48481 | tttataaatt ggagactggt attgtataat ttagttcatt gaaataattt gatctggtga |
| 48541 | cttcatagcc agacagagtc aatggattca tataaatgag aatccagggt taatcaccat |
| 48601 | caaacatttt caacataaag tgacattttt ttccttagaa tgggattttt actacattca |
| 48661 | aattttacta atgctgtaaa catgaaaatt ttgagtcagg taatgaattt gagaatggat |
| 48721 | agctaatctt taaaaatcta gtaacaaatg tttctgggaa ttgtggctat tgttcccttt |
| 48781 | tggaatgatg accaacattt ttggaaatga aaatattgat aaatttgtgt ggacttaaat |
| 48841 | atggagtcca cacaagattg gggtcatagg acagaaacaa tatttgtttc acatttacat |
| 48901 | tgctgaggta ttatgaagag gttagaaagg tgatttttat ttagcttgca gaataaatat |
| 48961 | tttaaaatca cagaataaca tatactatta aatgcctaca tacatttata tccatatgaa |
| 49021 | taaaacagta gaagctgaaa tataaaataa catgccactg ctgctggaga gatttaagcc |
| 49081 | aattatctgc tatcctggag catcagacat atatatctat atctatctat aatattatat |
| 49141 | atttcataat ttatatattt atatatcggt ataatatatt gttttataca gattatatta |
| 49201 | ttatatgctg tttttcacca abittgttta catatattat acattattta tatccggtga |
| 49261 | ctacataatt gtgcatggta catcccgtct ctagtcagtt gtcatgacaa gagttttaat |
| 49321 | catttatgct tactttatga ttctattgta caatttttat tggacattat ttcagttttt |
| 49381 | attaattaac agggcatatc aaatacctcc atacacactt tattacagat ttttttgtcc |
| 49441 | caaatatttt tagcattttt ttaaatatga aaatagatag tttggaaaca agcttctatc |
| 49501 | atctactatt tcttcagtta tttgaatgca gatggcctat cacaggacac aagaagttga |
| 49561 | gaatgtgcta ggagaattgc cagcactgct caatacaggt gtttgcccag gtctaccttt |
| 49621 | tgactotctc acttgatctg ttottgtgct tctcactttc ctgggcagcc agtcccttac |
| 49681 | tgagtatttc tggccttcag tatctgcttc ttccggttcc tttgatgcag cacccacttg |
| 49741 | ggctcgcttc tgtagttcgc taaactcagt tgcttatcat tgtaatattt ccgtaatatc |
| 49801 | agaatgacta ctgtctcaac ttaggagaca tccacttgtc ttctccagca cctgactgag |
| 49861 | ttcttgccat ataaattttt gttgaataaa ttcaatgttt ggcttttcac atcatcataa |
| 49921 | acaagactta caccaacaat catgggggaa tttgaataaa attaagatta ggtctgactt |
| 49981 | ttgcaatttt acaacttaca agttagtgtt aagaaaatgt ataaagatgg tgatatgcca |
| 50041 | ttttgaaaca tattaagcat tttcatcttt agaacagcct tcaagagagt tattatgaaa |
| 50101 | cacatttaag cttaagaaag ctgagaatca gaatgataat taataattat ttgagggtgg |
| 50161 | atgaaaaagt aaagaacatt tacacagttt tagtgagaat gtaaattagt tcaatcctaa |
| 50221 | ggaaaacaat atgaaaattt ctcaaagaac taaaaataga actacagttt gacccagcaa |
| 50281 | ccccattact ggtatgoggg tggtactata taagaggaaa agaaacatcc tgtcaaacag |
| 50341 | acatctgcac tcatgtttat cgcagcacta tttttaatag caaagtcatg gaatcagcct |
| 50401 | aagttcctat cagojgggga ctggatacag aaaatgtggt acatacacac catggaatac |
| 50461 | tatgcgtccg taaaaggatg aaatgatgtg ctttgcagca acatggactc tgctggaggc |
| 50521 | cgttttccta agtgaattaa cacagaaaca gaaaatcaaa tacttcatgt tctcacttat |
| 50581 | acgggagcta aacagtggtc atgtaagcac ataaagatgg aaacagtaga cactggggac |
| 50641 | tccaaaatgg ggggaaagag ggaggagaac aagggttgta gaagccaaat tgaagcctga |
| 50701 | aacaatacac ctttgtaacc cacctgcaca tgtggcccct gaatctaaaa taaatcaaac |
| 50761 | aaaaaattaa caagcaaaca aaaaacagag gaactagggt ttgaattaat atttctgatc |
| 50821 | tgcatatcgc tattttcctc ttaaattatg tgcttaaata catgaagaag gaggaggaga |
| 50881 | aaaaaacaac ttaattagga gacttacttt aataatgtat catttataac ttttcttcca |
| 50941 | aaatctggtt acatgtcaag aaactgtata atgtgaatct ctctagttca cctttccttt |
| 51001 | cctttatgta gatcttggtt gcaattcaga ttttcttttt tctaatgaac agtacaatca |
| 51061 | gtaacagttg caccaaacat ttcaatttgc aatatacatg taatggtttt tcaagtgaag |
| 51121 | gttacatttt atcttttttt attatccata gaataatgtg ttttaggatg aaagagacac |
| 51181 | tgtgtgtgtg tactccgttt aaaattaaga tctgacggtc tgagacatca catacaaagc |
| 51241 | ataattcagt agctatctga cagtttccca attgctatga cactaaaaat aaaccaaacc |
| 51301 | agataaatca accccctaag tggactctct ggcctgaaat gttojggttt taatgtgcct |
| 51361 | gtggaatgtt ccaatataat tttcatcaat gttgattgtc cagctcacac agccctgggc |
| 51421 | actagagtga ttgacataa ccccgtcatt attccaacaa ttaaatcagc aggaattagc |
| 51481 | acaaacattt ttttcctgtt ccgtacacta aaaatgtttc catgctgcaa tgttaggatt |
| 51541 | taaatgtaac aaacataatc ttccctgtgt tgtataactt tccgatctac taagaaagcc |
| 51601 | taccaaaatg gagcatttgg tttaaaaata aatacctgga ctcttccatg tggcaacaaa |
| 51661 | caatacataa gagatattgg acaatgaaca attaattaca ttgataaaaa taattgatga |
| 51721 | attaagaatg taaatctaaa gatactttat attcctcttt ttggtttaac tttcaaatcc |
| 51781 | agaaagctct taacctggtt aactaatcga tgataacgtt atacaacaaa cttttgttga |
| 51841 | gtacttgcta ttcattcctt gaacgttatt tattgtaata aacgtgttaa ttgaggttct |
| 51901 | acatatataa aactgtttaa ggtgcaatga aacaaaaatc acctgaactt cggacaaatg |
| 51961 | tgatttttgt ctttgcttag gacgtttttg gaatatttac ttttgaaaat gtctccatga |
| 52021 | cctgtggcag tgttttcttc tgaatagtat aaatgacgat ggcaaatcta aaataagtta |
| 52081 | tttggagcca catctagaaa ataatgtgag tattccagtt attagggaag gctagatttg |
| 52141 | atcaaatata tatatagtta tatgtattct ctctctatat atagaatata tagttacata |
| 52201 | tgttctatat atagttatat atactctcta tatatagaat atatagttat atatactctc |
| 52261 | tatatataga atatatagtt atatatactc tatatactct atatagaata tatatataac |
| 52321 | tatatataga atatatataa ctatatataa atatatagtt atatataact atatatagaa |
| 52381 | tatatataac tatatataaa tatatagtta tatatattta tttataaata tagttatata |
| 52441 | tactctatat agatatatac tctatataga gaatatatag ttatatatat tctatatata |
| 52501 | tattctatat atagttatat attctatata tagatatata ttctatatat agaatatata |
| 52561 | gttatatata gttatatata ttctatatat ataacttcta tatatagtta tatattctat |
| 52621 | atatagaata tatatataga atatatgtat agaatatata gaatatataa tatatagtta |
| 52681 | tatatattct atatatcgaa tagttatata tattcgatat atcgaatata tagttatata |
| 52741 | tattcgatat atcgaatata tagttatata tattcgatat atcgaatata tagttatata |
| 52801 | tattctatag ccataatgcc tattagcctg gcatttagac ttgcaagtgt caacttatta |
| 52861 | cttaccttgt tttattttat ttctttaatg atctcaaaac cttcctctct cgatttcaat |
| 52921 | ctgtggtttg ttcccaccat ttgccgaggt ctagcatctt attctttgat tttctttttt |
| 52981 | cttttcttca acttttaagt tcaggggttc atgtgcagga tgtgtaggtt tgttacgtag |
| 53041 | gtaaatgtgt gccatggtgg tttgctgcac agatcatcct atcacatagg tattaaaccc |
| 53101 | agcatccatt aactattctt ccgatgcgtt ccctaccctc atacgctccc cccacaacag |
| 53161 | gccccagtgt gtgttgttcc ctcccatgtg tgcatgtgtt ctcatcattc agctcccact |
| 53221 | tataagtgag aacatgcagt gtttgctttt ctgctcctgc attagtttgc tcaggatgat |
| 53281 | ggcttccagc tccatccatg tccctacaaa ggacatgatc tcgttccttt ttatggctac |
| 53341 | atagttttcc atagtgtata tgtaccacat tctctttatc cagtctatca ttgatgggca |
| 53401 | atgattttgt aatataatga tttatattcc tttgggtata tacccagtaa tgggattgca |
| 53461 | gggtcaagtg gtatttctgc ctctaggtat ttggggaatc accacactgt ctttcagaat |
| 53521 | ggttaaacta attaatactc ccacaaacag tgtaaagggt tcctttttct ccacaacacc |
| 53581 | accagcatgt gttgtttttt gacttcttaa taatagccat tctgattgga ataagatggt |
| 53641 | atctcattgt ggttttgatt ttgcatttct ctaatgatca gtgatattga gcttttattt |
| 53701 | catatgtttg tcagctgcat gtatgtcttc ttttgagagg cagaaatacc attcgaccct |
| 53761 | gcaatcccat tactgggtat atacccaaag gaatataaat cattatatta taaaatcatt |
| 53821 | gcccatcaat gatagactgg ataaagagtc tctgttcatg ttctttgccc actttttaat |
| 53881 | gggatttttt tttcttgtaa atttaagttc cttgtagact ctggatatta gacttttgtc |
| 53941 | agatggatag attgcagaag ttttctccca ttctgtaggt tgtctgttaa ctccgatgat |
| 54001 | agtttctttt gctgtgaaga agctctttag tttaattaga tcccattcgt caatctttgc |
| 54061 | ttttgttgca atcgcttttg gcgtcttcgt catgaaatct ttgcctatgc ctgtgtcctc |
| 54121 | aatggtattg cctagattat cttctaggat ttttgtagtt ttgagtttta catttaaatc |
| 54181 | tttaatccat cttgagttaa tttttgcata tgatgtaagg aaagggtcaa gtttcaattt |
| 54241 | tctgcatgtg gctagccagt tctcccagca ccatttatta aatagggagt cctttctcta |
| 54301 | ttgtttattt ttgtcaggtt tgtcaaaagt cagatggttg taggtgtgtg gctttattcc |
| 54361 | tgggttctct gttctgttcc attggtctat gtgtctcttc ttgtaccagt atcctgctgt |
| 54421 | tttggttact gttgacttgt agtatagttt gaaatcaggt agtgtgatgc ctccagcttt |
| 54481 | gtgctttttg cttaggattg tcttggctat ctgggctctt ttttgattcc atatgaattt |
| 54541 | tttaaaagtt tttctaatgc tgtgaagaat accaatggta gtttaatgga aatagcattg |
| 54601 | aatctatcaa ttactttggg cagtatggcc attttcatga tattgattct tcctatccat |
| 54661 | gagcatgaaa tgcttttcca tttgcttgtg tcctctctga attttttatc agtggtttgt |
| 54721 | acttctcctt gaagaggtct ttcacttccc ttgttagctg tattagcatc ttattctttc |
| 54781 | aagaggacag tttgttcagt ttgtttaaat tattatcacc atcacaacca ttaaaccttt |
| 54841 | attcagcaac tgaagaatgt accacaatat ctctcatttg caagacatat tctgttcatc |
| 54901 | ttctgctttc tgcttctgta gcttataaag atatataaat atattttaaa cccaatttgt |
| 54961 | aattatatat gatgggaatt gaaaacgtgg gtgattttca aataatcact aaggaataaa |
| 55021 | atatgctaaa aataaattgt gaagaattga tatgattttc taaggatggc tttactggag |
| 55081 | aaaagcagtt atcctacaat agttaatata taactatttt tatgattatt ctgttcagaa |
| 55141 | tctgaattta tattattatt tggggccttt tttaatcaag tgaagaaagg tatattttct |
| 55201 | tttcaaactt tttattttga acattcataa attacatgaa cactaaaaga cataaattat |
| 55261 | ctttcaggaa acattgaata aattctggat tcttctatat tttattgatt tgtaaaacat |
| 55321 | ctcaaattta acatagaaat gttaaaaaga gattatatta taacgtttgt tggaaaatat |
| 55381 | gctatattta taatatttat tctgtattta tatattataa atatattcta tatttatata |
| 55441 | ttataaacat attctatata tattataaac atattctata tttattaaca tttaacatta |
| 55501 | atgttaataa atatagaata tttttcaaca aactttataa tataattccc tccttcaatg |
| 55561 | aactaatttt tatggtggtt tacatttctt tttcaatgtt ggtttacatt tgacgttagg |
| 55621 | atgtttatta gttaatacaa taaagtgggt ggtcttttca ttaatattat cagtcaggtt |
| 55681 | tgtaaatagt gtatgttttg tataaccact taaaatttat atactgatat atattttgtt |
| 55741 | taagaatact taaaataaaa acattagtta tcatttgttg aactggaagg atcattactt |
| 55801 | aaaaatccat ccttaaaatt aaaatgactt atgcttcact gtgtagtgca ttgataaata |
| 55861 | tttagtctat agttttttct attgctatgt gtttagccaa gaattgtttt taatttggtg |
| 55921 | aacagtacct tattaggaat atgaaaattt ttgaagcagt catgaaatat ttaacaatta |
| 55981 | attatttaac aaacaattat gcaaaattaa ttttactaaa gaaatatttt taaatcactg |
| 56041 | caagaaatat tttcattcaa atgagtactt acaaccaaga aagagtcttt tattttttaa |
| 56101 | atgtgtataa atttaaactt caaaatactt aaatcactta atgcctcaga gaaaatatct |
| 56161 | taatacttat gaagtgttac gtgtactaag cattatgtta aattctataa agacatcttt |
| 56221 | ttaaatcatc acaagaaatt acaaggcagg tgcatctatt atatttttta tagtagaact |
| 56281 | aattgaggca cagtgagagg aaatgatgta ctcaagttcc acaggtgata agtggtacag |
| 56341 | ctgagtaaaa ttccaagtct aaaagctagt ctcttaacta ttatatatcg gcctatatgc |
| 56401 | ataggccgac aagtagttat ttaaagttat ataaatcaag agaaaacact acaaaattta |
| 56461 | cctataggat atttatacaa aatatgggca ttttattagg aaagotttct agaaatatgg |
| 56521 | gggaacttta agaaacagtg aaattcaaag ctatcattgt ccaaaagcat gtggtcttct |
| 56581 | gtttttgcac tagtgactta ggtgcaaatt acttccaaga tttttctcct aattgagaag |
| 56641 | tactttttag gtctagattc catggaggag gtgggtcttc gaggtggggc ttcaaggtgg |
| 56701 | agatagtgtc ttttttctag catgtaatga ggtaaatttg cttttgaatg aacaggtgca |
| 56761 | cctatttcaa ttaaggcaaa ttagtttcca aaggctttgc tatttaagat attatagaag |
| 56821 | gctatttaaa tgtgccaatt tacataaaat acattttgaa agattacagg aaacaatttt |
| 56881 | atataattca ctgaaacaat ttcacagtcc tctcaaatga tgattttgaa aatgacctaa |
| 56941 | atggcaggca cctctttgaa gcagctggca tgatgttatg tggtcccatg gccccttaca |
| 57001 | ttttcaggtc aatggtaaat tctccagagt ccaacttcaa ccctgcagta ttctgttcaa |
| 57061 | aattgcccct gttcagaagc ctgcatatgt aacctcttac attttgtgtt tcacaatttc |
| 57121 | cattgaactc ttcctacaca ccagaatggg caaaaatagg aatttaattt ctgggaagat |
| 57181 | ttcctctaag agcctctgtc caaaacatcc agtgctgaaa caagaaaagg tggtcctggc |
| 57241 | atatatccac tagtaatttt tttgtcttct tcttttcttt cctgttttat gtgtgaatag |
| 57301 | tgaaacttat cggttcaaag accgatgagt cagaatgctg tcattttaca tccaaacagt |
| 57361 | ggaaatttaa attgttaaaa taaaaataca caatcactaa cattaccctg atgaaataat |
| 57421 | gataagcaaa gtagtcttaa attacaggct ataagtgctg gataaagaac aatattttaa |
| 57481 | aaatatctaa agtcatataa tgaacagaag acagatttcc atttatacct gaaagactgg |
| 57541 | ttttcagaaa ttataataga acatggggat ttaaaaattg tatttggatc atttatttta |
| 57601 | cagttagaaa gtgatgtagg aaggagttcc cagaggagat gaaacagatt gaaaacagca |
| 57661 | aaattccatg acagaggtgg cttgaaatag accctttttt caaagcaata actggcctct |
| 57721 | tcatcatcta tagatagcat ttttggcaaa atcagaaaca tttgctttgc cagtcattga |
| 57781 | aggtaaaaag aaaaaaaaaa tggctgaatg cttttaaatt acatttattt aatttctgct |
| 57841 | ttcatacaaa ggcataaaat tatcagttta aattttaaaa aatattatta taaaattaca |
| 57901 | tgcttagaag tctgttgatt gttttcccac agccatttta taactgaaac cctcaatttt |
| 57961 | ggatatgaat gtataataaa cgcacatgtt cattctaaac attttctttt caaatacttt |
| 58021 | tacattcctt taggtttcct aaagaaatgt aaattccttc tgccacttag tttttgctat |
| 58081 | ttttagcagt ttcttaaagt ctgcaaattc ctttcactca gtttttgcac ttagttcata |
| 58141 | atgacttcac atttgacatt gattgtccac aaaaggggtt atttttagtt tcaaaaggtg |
| 58201 | aacgttattt gttttagaaa ttctcgcgta tatttcttag tctaaagata ggttttctaa |
| 58261 | cataaaaatg tgatagtgta gtctgtttgc tgagcacttg tagtggaaca aactcagtgc |
| 58321 | taaattcttg gtttttaata actaatttat ttattacagt aactgctaaa tatgcttcaa |
| 58381 | attataaact atttgccaaa attacagata ataatataac caaacaccat gtggatttca |
| 58441 | cctaaactag aagtaacttg gtgactttct tatttaatct tttatttatt gtacttggcc |
| 58501 | atacttattg tatgtttatg tataaaaaca taaagcaata gaagttttta cagttattat |
| 58561 | actcactgtc ttataaatgg agaaactgag acccagagaa ttttagtaac ttattcaaag |
| 58621 | ttacctagct ggtgactaga agcaaaacac aaataagacc acagattact agttttgtca |
| 58681 | agatcttcaa cataatttaa tattgcbgtc aactcagagg tctcagaaga taaggtctta |
| 58741 | ccatctgaaa acaacaaaaa agaaatttca taaaatcctg tttgtaccaa gcaatttatt |
| 58801 | tctagaatga aagcaaaatt atttttttaa agttctttag attgctgaga aatcaagagc |
| 58861 | attagggttg tgtctcccct ttaaatttac cttttagttt ctttgggttt aagtttttag |
| 58921 | atgaaatttt aaattctatt tagctctcag tttatgtgta tttaaaattt agatttagga |
| 58981 | tctattcttt ttaataaggg tgaaattaca tatggagaaa tttactgttt atagcgagtt |
| 59041 | ttgacaacta gagttgtata accaccatcc ccacagtcaa gatacatcac ttctcccctt |
| 59101 | cccaaacata ttccctggtg acactttgtg gtcatactca ttctccgcct ctagcctttt |
| 59161 | acaatgactt ctgttactcc ccctgtagat ttgcctatta aagaatgtta tataaaggaa |
| 59221 | tcacacatca tgtagcattt tgaatctggc ttgctttact tgttttaatg catttgagat |
| 59281 | ttgttcctat tgtggcagat atcagtagta gtcttctttt cattgttgga tattccatta |
| 59341 | catgaatgta caatcacatt ttatccattt gcccagctaa agaacatttg ggtgttttca |
| 59401 | gttttcatta ttatgagtaa accctccata aacatattgc atacaaattt ttatgtgaac |
| 59461 | ataagttttt gtttcaccta aacagatact atacaggact gaaattttag taagtatatg |
| 59521 | tttaactttg tgagaaactg cccaactgtt tttgcaaagt ggctgtacag atttacattt |
| 59581 | tcaacagcaa catgtgagag ttccagttgc tgcacgtcct tgtgagcact tagggttgta |
| 59641 | tttttaattt tatccactct aatgggtgtg gagtggtgtc ttattaaggt tttaataagc |
| 59701 | atttctctaa tttaatgcat tatattactt ctttttgttg gtttgtaaac tgcttatctt |
| 59761 | ctttggtgag tttgatcaaa tcatttgtcc abitttgtgt ttttgattat atttcagata |
| 59821 | caagtcttta ccagttatgt gctttgtaaa tgttttctca ctgtttatca tttgtgtgtt |
| 59881 | ttatggaaag cagaatattt cgattttctg gaaatataat ttgtcacttt aaaaattatg |
| 59941 | taaattgcta ttggtattaa gtgtaagaaa actttgcttc atctatgatc acaaatattt |
| 60001 | tcttctgaaa aaaaagctct ctgaagagtt tttttttttc aatgaatgtg ttttctttct |
| 60061 | tctctccata tgttatcttc tgaatagcta agcaatgttt acattattta ttcaggcctt |
| 60121 | tccatccact ttcacggatt ccactgaagg agaaattggg tttgaaatcc tcttttctca |
| 60181 | aaaactaatg agtcatgcat cttctcattg atattatctg gctcccctga aaacctctag |
| 60241 | tttggtccat ttttagtgat atctttctac tcaggttcct caaaccaatc tgtacctaag |
| 60301 | agacccccag attaaaattc aagttcattc ttttaacaaa ttaggtgcta attgctacaa |
| 60361 | atgcaagaaa acttcaggtg ctaatttgtt aaaagaatga acttgaactt tagtctgggg |
| 60421 | gtctcttaga tatagattga aaaggattga aaagaattat agtttctttc acttttgcca |
| 60481 | tcatacaata tgtattattt atatttcttc ttcgttaata ttcagttcac cttctttgtc |
| 60541 | catctttcta ttgaagcacc agtattcatg cagtgtttta agaatattag tccattttta |
| 60601 | tccattatgt atattgcaaa taatttcgta ctttattgtc ttttaatttg tttgcagtgt |
| 60661 | cctaagagta taaatataat agcttttcta cttttttcat tttttaatat tgcattttct |
| 60781 | ctattgtgaa tgttatgctt atgtttttct ccagctgatc tattgaggaa cagtgttttt |
| 60841 | ttaataaata taaatattgt tatagttatt tctcatttcg aagttttacg gtactaattt |
| 60901 | gtctgtctta aaacttcaaa taatacttaa ttgaaagtct ctgtctacca tcatcccttt |
| 60961 | cttaacagtt cggtgaatat cttttattat attgtgtaag gatgttgtat tgcagctcct |
| 61021 | cttgctaggc tgaacatgca gacctgttct ggaaatagat gtgatcagtt taggtcacaa |
| 61081 | cctgtcttga atcacagctg acttgtagtg gagagttcca ggaaccttcc aatcacacac |
| 61141 | aggtccctat tgctcctttt cttctagggc tattcaacaa gcagagacaa acaaaaactg |
| 61201 | atctgtcaca ttttactgca ggtaacagtg accacaaata cacctgtaca ctgttttttt |
| 61261 | tccaacatta ttgaggtgtg actgacaaat aaaaatggta tatatttaag gtatgtgatg |
| 61321 | cgatgttttg ctgtacatgt acattgtgaa atgattatca taatcatgtt aacatatcca |
| 61381 | tcacctcaca tattgttatt tttataatca cttttataaa ttttcccttc agtagtaacc |
| 61441 | ttttgagatt ctgaaacact tttgagtttt gaatgttgtg tcataagtga gtttttttgg |
| 61501 | caaatatttt ataaacatct aattgggaaa ctgtgagagg atgttttgta actttcattt |
| 61561 | aaactaagat ctcctctcca cctccaccct tcccatattc agtagatttt ctatggctgt |
| 61621 | atggtgtatc aggaatcatc ccatatactt cagaaagtaa tggaataata taaatatttt |
| 61681 | tctaacatag acctacagag attaagtgat atactcaaat cataccataa atggtagtga |
| 61741 | aagggtaaac tcaacattca cagccaactt cattatctaa tagggaacaa cacatttcat |
| 61801 | attaataatt gacttttatt tttaggaggc tctgttataa tgaaattaaa aaatgagatt |
| 61861 | agattatgtt tctcaatata tggtacaaaa atcacttaca tagaataact tcgattatta |
| 61921 | aaaatagaag tgaaaaatac ttttcagatt aaataagatt tcaattttaa ggcaaatatg |
| 61981 | ttcattttgt gctcaaaaat tagacaggaa aactataatc tgttaaaatg taggttcttg |
| 62041 | atccacccaa gaaaccatgg agaggtgggg gacagttacc tgcatttctg acagttagtt |
| 62101 | cattgactct tattcgacta aactttgaga accagtgtcc aaagtattta cttacatatt |
| 62161 | tatatttaaa agctgggata atcatgacaa tataaaataa acaattgatg ttgtatttta |
| 62221 | cttggcagat agataaagct atacaagttt cctttttcct aattctaaag cataattaga |
| 62281 | aaaaaattgt atttatttct tagatatggt aactactaaa atataatttg gaatgttcta |
| 62341 | cttaaatgag tatacttatg tatgcagtta gccgtcaata agatttagtg ttctctgaac |
| 62401 | tatttcattt ctatgaaggt ggtattaatc ttgatgataa aattttcttt taaatattga |
| 62461 | gagattctgc caaaatataa atcttactgt gacaatgttg ttgaaattcc tttcttcata |
| 62521 | tgattatttg ttctaaaata gctcccatga aagtaaaatt cctactctca agcatcctgc |
| 62581 | ttaagcatat tgtattgttg ctggtaacta aaaataaagc aaagcattag ggtggatttc |
| 62641 | agtgtaattt tatctgttta gtagagtgat acaaatgaat aaattttcaa acatacaaat |
| 62701 | tgaccaatta tttgaaagag ctatgaataa aataaataag ctaccttaaa attattttaa |
| 62761 | aattatatca caaaattttt ttcctaacaa caaaatatgt atttttaatt gttttggttc |
| 62821 | atacaaatta aaatacaaat ataattttac caaggaaagt gaagtaattt aggagtaatt |
| 62881 | aataatattt gtgttaggga acggagcata gatactatta aacctaaccg ttttatagtg |
| 62941 | catttgtttc taaatccctt aagctggtaa gtcattgatc ttgcattttt ctttgacagg |
| 63001 | cttatgcaag caaagatctg gaggagcagt tacggtctgt gtccagtgta gatgaactca |
| 63061 | tgactgtact ctacccagaa tattggaaaa tgtacaagtg tcagctaagg aaaggaggct |
| 63121 | ggcaacataa cagagaacag gccaacctca actcaaggac agaagagact ataaaatttg |
| 63181 | ctgcagcaca ttataataca gagatcttga aaagtaagta tgggaaataa aatgtatagt |
| 63241 | aagccttata tatcaaacca gtagaagttg ccagcctggt tctttaattt cagtgtttgt |
| 63301 | atcaccgaat acacctttag ggcggaaagc aaatagaaaa taaagaatta cttcaaaata |
| 63361 | caacatttta tgatttcact ttgagtatta agatgacttc ataaaataca ggatagatag |
| 63421 | gtccatgcat aaacaacaca catttatttt ttcaggtgat agccattgga aaatttttaa |
| 63481 | aacagatggc tatattttac atactatatc tgtttaataa cttaaacatt ctttatctta |
| 63541 | tcaaataatt ataccttaat ttcctatctg atttggattg agaaatgctt catttgaaat |
| 63601 | caccttttca gatgtcactt gttgctttgc tgcttcacag ataactggct cagagcggga |
| 63661 | cttaagctga tgaggtatct agcataaagc ttcacaaaaa atgatcatgt tgcataaaat |
| 63721 | gtcatagcat caattggtgg ggtatgactg ggcacagagg ttcatgtctg taatccgaga |
| 63781 | ccattgggag gccaaagtgg gaggattgct tgaagccaga gtcagagacc agcctgaaca |
| 63841 | acacagcaag acctcatctc tattaaaaaa aattaaaaag tggtggagta atttaaaaac |
| 63901 | atatttgcct tcagttggta gctcttaatc gctataaaag tttgatattg gaaagtattt |
| 63961 | tgagaacaga ggcaaacaaa atggcttcat ggttgtataa ccaatagttt atgtcctttt |
| 64021 | ccaaaaattc tattttaggg gaaatatggg ataagtaaac tagtatactt aggaagtaca |
| 64081 | ttatttgtaa tgcagaattt aaaaagtcaa cagtattata ggtaagctac taactcatca |
| 64141 | gaaacaaaat tccagaggag agttaaaagg gataggaaat attgactttg ctgaagaaag |
| 64201 | ttcagaatga gtcccagttg gtgaaaaata gccttgtatt ttatgtagct gcagccacga |
| 64261 | cattactcaa ggttgccaaa tagtccgcct gaaaaggacg tttgacttga atctaaaacc |
| 64321 | atagcbgtct tctcatatgc aactcagttt taaagaaaaa taaaattata tttatatttg |
| 64381 | cattgagagc acaaaaatgg cccatactat agagaatgaa catgactctc tctgcacatc |
| 64441 | ttataaacat tcatatatta aattaccatt ttcctaagaa agaaaaacca aacattagtg |
| 64501 | ttgtgggata ttggtgtact tttaataaag cataatgcta atttttctta ttgcctagct |
| 64561 | atagaaatga gtttaagaaa tatccttagc ttgtcttttc aatgttatat cattctcatt |
| 64621 | cacaagtcaa tttaggatcc catcagctgt ctctttagtt atatattgaa cttaaatgtt |
| 64681 | aaaatctgaa atgaatgtga gtgatttgca gctgcbgtgg gcttttttta tcttggtggt |
| 64741 | ttccgtaaaa cttactgatt tccctatgac ttgacaggta ttgataatga gtggagaaag |
| 64801 | actcaatgca tgccacggga ggtgtgtata gatgtgggga aggagtttgg agtcgcgaca |
| 64861 | aacaccttct ttaaacctcc atgtgtgtcc gtctacagat gtgggggttg ctgcaatagt |
| 64921 | gaggggctgc agtgcatgaa caccagcacg agctacctca gcaagacggt gggtattgcc |
| 64981 | cattcctgct acctgcgaga gcccccttaa tcagaagctt taactttggg gatgatgtgt |
| 65041 | ttaaacttct ggttgcttta aactaacttg gttcaactca gggtctcata ttttctttag |
| 65101 | gggaatgaag tcagtttttc agtgttttgt gtgtttgaca acacagtgtc acaaagaaag |
| 65161 | taattgatgc tttattactt tgcaaactct tcatatagtg gagtcaaagc ctcatgcaga |
| 65221 | aaacaataaa tatacgtatg tatgtgtgta catgtacgtg tgtatgtgtg tgtgttgatc |
| 65281 | ttcaagaata aacatgttca tttggcaata aaatactttt aaatgggtga gttttcagat |
| 65341 | ggtggagata acagtgcaaa cagtattcaa agtgtaaaat taaatgcgtg tgttctgtga |
| 65401 | aacagtggta tagtattgaa ataagccatc tcactgaaga aatttattgt cttcctgtcg |
| 65461 | aaagcaaagg aaataactct gacactaaat ctcagatgtt ttgaataaac tgggaagttt |
| 65521 | atctcattaa atggtaagct tctgataacc aggaaatgtc tcttaaggtt ggctttggat |
| 65581 | cagggatttt cagaccagac acataaaccc gotttgtggg cottacgtgg tagaggaggc |
| 65641 | tggcbgtgga acacgcgtat gtgctggcga taaccatgaa agtgttgttt gaaattgtgt |
| 65701 | gcacctctgg cctctgaaca gaaatgtttg gagaaatcca gtcccgtggg ctggactgaa |
| 65761 | gcaaacttct cccgaaagac aaaactgtgg ttgtcctaat gacctttcta agctattttt |
| 65821 | ggagttagac tcacacacgt aaatctggct aaattcagaa ctgttctttg acctggagaa |
| 65881 | tgaaaaatga aattgaaaga tgtggtgtta gatttgattt gtatgatgtg gtatgacaga |
| 65941 | aaattcacat tttaatttta tgcaaacttt agctgatcta catggttttc agtaataaaa |
| 66001 | cctactctta tttcaatata gtattaatag aaatcagagt taagattact atctcaattc |
| 66061 | catgaactac attttttcag ctagctgaat atttccaaaa tttttactgt tacgatcatt |
| 66121 | tggcctctga gtttggagac attgtcttat tatacaaccc aaattaattg ctttcttttc |
| 66181 | aaaaattgag tttctttata ttgctcaccc acttgataaa gaaacaagcc attttagatt |
| 66241 | aagacttgca agaagaaaca aaaaataaat tataaagcta aattttctat tcagcttgcc |
| 66301 | tctgcctcaa ctctaaaggt taaaaaaaat taaaaggtga tgctttgggg cctgctatca |
| 66361 | tttcttttag tatcatagtt agttaagctt agccagatgc aattcattct gattatttcc |
| 66421 | taaaagcaca gcaaaataat gtaggggtcg ttccaacatg tgttctcaag gaatacaatg |
| 66481 | tgactctaaa catagtcctg catttagaaa ctttaccaga aaaatgtaca gttgtaggaa |
| 66541 | catgacaata tttcagtggt tgccaaaatt ataatatttt tgatagacta acatataatt |
| 66601 | atactattgg acatagcaaa agttatcctt agttcattta tcctccctga gtctttatca |
| 66661 | aacaacaata aacaataaga cgttatagtt ttgtacttac tattctaaat aaattaatac |
| 66721 | tactttaaca ggttttattg gtattatgta ttttatcata accctgaaat caaacaggat |
| 66781 | tctttaagat cctctaaatt ttagaactgc atactttaaa ataaattaaa tagaaatact |
| 66841 | taggccaagc agatattctt tgaaaggtaa tattcatttt taaaacatat taataattaa |
| 66901 | aabittgttt ttatcttcaa gccaaacctc ttaatgggaa ttttggtata ctgagtctct |
| 66961 | tgagaaatat gtcatttcct tttaaagtta gagaaaataa atatggatga tttaggaatt |
| 67021 | gaaatataca atttttggaa tgccgttaat gatttatatt ttaaaataat tcagttaagc |
| 67081 | aatatgatgt attctagatt atattcctaa tatggtattt gatttaaatc aaatagtaaa |
| 67141 | ccaaggtact ttggatactg tttccaacta ataagataac cttctctctg ctcttcctcc |
| 67201 | ttccatttcc tttcttcata atcttttcta taaattactt tcaagcatat tttttaaaaa |
| 67261 | catagatcca aatctatggc taggcatggt ggctcacgcc tgtggtctca gctgctgagg |
| 67321 | aggcagaggc aggaggatcg ctggagccca ggagttcagg gctgcagtgt gctaggattg |
| 67381 | caccactgca ctcccagcct aggtgacaga gcatgaccct gtttcaaaaa caaaaaaacc |
| 67441 | ccacagagct gatgtcagtc tccagcataa aggcctttca gtgatttccc attgcttgta |
| 67501 | aaataagaca aaaatctttt ttgaaggtta tatttattga catatcatag ctgtacatat |
| 67561 | tttggggtca tgtgttatat gttgatacct gtacaaaatg tgtaatgatc aaatcagggt |
| 67621 | aattgggata tccatcatct caaacattta tcttttcttt ctattgggaa caatacagta |
| 67681 | cttctcttgc agctttttaa aaaatataca atatatcgtt attcactcca atttccctga |
| 67741 | tgtactattg aacactaaaa cttactcctt ctatttaact gtgcttttgt acccctcaac |
| 67801 | catcctctct ccatcctccc tacccttgcc cctcccagcc tctgataatc agtattcttc |
| 67861 | totctacttc catgagatcc actcgtttag ctcccacata aatgagaaca tgcaggattt |
| 67921 | tcaagaccac atctttatca caacctgcaa tctctgcccc atagcccctg ttcagcatca |
| 67981 | tctaatgtaa tgattcttga tcttgtgctt ccagtgctct agactttctc caattcctgg |
| 68041 | gcatcatcgt gtttcctctg ctcctgggcc cttgcacaca cacctttagg ttaacctggc |
| 68101 | cttcctgtct cctctttcct gaggtgctag ctcttcagat cttaatttgg gtgtcaagtg |
| 68161 | gctttggcaa ggcctcaggc aagactttct gacctatttg gattagaccc aatatttgta |
| 68221 | gtaaatgctt tatagtttcc catattttta aaatagtatt tattacagcc tataatcaca |
| 68281 | tagtttatag ttatgtagtc attgttactt gattaatgtc tccctcctcc acaaaggcaa |
| 68341 | aaacagatcc gttcactact tatcccagtg cctaccacag aatcagtgca caaatctctt |
| 68401 | ttagtatcac ataaaaactg tggacccttt catgagaaca aatgaaaata tagacacatc |
| 68461 | cacagcattt ccccccacag ttttagggag tttgttccct gacccctagc cttttccctt |
| 68521 | taaaccaggc tttaaaactg ctgccttgaa gattattaat aataaaatag ctgattaaca |
| 68581 | cacttgtgga attttcaaat aattggtctt tttgctaaac aaacgtttat gctatcaatt |
| 68641 | tagcttgatc ttttaaaaaa tatctgtttc tggagaggaa cagaaaaggt aactattggg |
| 68701 | tactgggctt aattcctggg tgatgaaata atctgtgcaa caacccccat gacatgagtt |
| 68761 | tacctatgtc acaaacctgc acatgcaccc ccaaacctaa aataaaagtt tacctattta |
| 68821 | acaaacctgc acatggaccc ccaaacctaa aataaaagtt tacctatgta acaaacctgc |
| 68881 | acatggaccc ccaaacctaa aataaaaccc caaacctaaa ataaaagttt atgtcacaaa |
| 68941 | cctgcacatg cacccccaaa cctaaaataa aaccccaaac ctaaaataaa agtttaccta |
| 69001 | tgtcacaaac ctgcacatgc acccccaaac ctaaaataaa agtttacctg tgtcgcaaat |
| 69061 | ctgcatatgc accgcgaaac ctaaaataaa agtttaccta tgtcacaaat ctgcacatgg |
| 69121 | acccccaaac ctaaaataaa agtttaccta tgtcacaaac ctgcacatgg acccccaaac |
| 69181 | ctaaaataaa accccaaact taaaataaaa gtttacctat gtcacaaacc tgcacatgga |
| 69241 | cccacaaacc tgcacatgga cccccaaacc taaaataaaa ccccaaacct aaaataaaag |
| 69301 | tttacctatg tcacaaacct gcacatgcac ccccaaacct aaaataaaag tttacgtatg |
| 69361 | taacaaacct gcacatggac ccccaaacct aaatataaac tttatatatg tatgtatata |
| 69421 | tatacatatg tatatacaga tatacatata tgtatctgta aaagaaacat atctatctat |
| 69481 | atctatatct agatatccag atgactagat gagatataga tatctgtttc tcttacagat |
| 69541 | aaggaagaaa attcttcttt attctctcat tcattagtta atatgagcaa aagccaacat |
| 69601 | tatatctttc ttactattgg tcaaccaaat aaatgcatac tttacatatg ggccaaatag |
| 69661 | atacctaagc ataccttagg taaaatctaa acttggaaaa cagaaaatca gtacattgtt |
| 69721 | agtaagcaaa gtaaataaat ttagtcttct attaaccatt tgagtttttt ctgtgaccat |
| 69781 | tgcatattca ttttctgcat tggtctaatt aggtgtcaca ttcacccagt gggtattgga |
| 69841 | gtgaaagtat ttattcaagg gtaggtgtgt atgctcagta gcataaaaat tgctctatca |
| 69901 | agagggaggt gttggagagg tttaggcttt agaaattgca aggcttcgct gggcactgtg |
| 69961 | gcttacacct gtaatcccag cactttggga tgccaagaca ggcagatctc ttgagcttag |
| 70021 | gaattggaga ccagcctggg caacatagtg aaaccctgtc tccccaaaaa agtataaaaa |
| 70081 | ttagccaggc atggtgttgt gcacctgtgg tcccagctac gcgggaggta gctgaggtgg |
| 70141 | gaggattgct ggatcctgga aggcagaggc tgcagtgagt aaagatcgca gcactgcact |
| 70201 | ccagcctggg tgacagagcc agaccctgcc tcaaaaaaaa aaaaaacaaa aaaaaaacaa |
| 70261 | aagaaaaaaa ttacaagggg ccaggggtaa gatgagcatt tagtgctaag tgtaagctgc |
| 70321 | atataggtga aataattttg cccagtctac ttagtactta aacatcagat atttgctata |
| 70381 | agaaaattgt ctactttaga tcacatggaa tcttttcttg ctgtatttca tctctttcac |
| 70441 | ctcccagctt tgcagaattt aggcttactt ttataataga tttacaatgt tgatatgagt |
| 70501 | tttgtcaact tcttcccaat catttgcttg tgtgaaaaat caggacttgc ccatgaaatt |
| 70561 | tctgaagttt gtctgctttt ttgttattgg ttgatttttt tttcagtttt ttttctatcc |
| 70621 | tattggtggg ctatatattt acaataatca caatatttat acattctgat ctgtaatgca |
| 70681 | gtcatatttt aaaatttata tagcattagt ttttattttg tcattttaat tttttaaaga |
| 70741 | tttgaagatt ttgagaatta aaaatttgta gttcatcaaa aatatggtat ttctcctgag |
| 70801 | aattctgacc taagacacct tctctgcaga ctgtaactct ccttgtattt tctcatctac |
| 70861 | ttttttttac ttttcccaaa cctttccctc caattcaggc ttctttcctg atttctatac |
| 70921 | tgtccactca accttgtgac ttggctgtcc ctcagacagg tcagtctgtc tcacttaaca |
| 70981 | gttagaacct attccctacc accttgccca aataagcaag taagtgaatc ctattctttt |
| 71041 | tccagtgttc bitgtcttag taaatgacat catcattcag ccagtoggtc aagccaagtt |
| 71101 | attcttcctt tcaccaaaac taattgctat tcaatctacc ttgcaaacat ctctaggacc |
| 71161 | tatccagtgt ctccacgctg ctaatcccgc tctgatctag gtcacattta ccccttacct |
| 71221 | agaccattcc tcttggacaa gcctaataac tggtctccta cctccattct tcctttcctc |
| 71281 | caatctattt gccacttttt agcagtgatt tttttttctt ttcttaatgc taagtcagat |
| 71341 | actgctaaaa actccttgat aacattgcct tattgggatg gagccagcat tcaactagtg |
| 71401 | tggtgataag cttatctttt ctaattgggt tgcttaggcc agcatctggt cagattgatt |
| 71461 | ggtacaaatg ttctaagttg ttagtgtttt gaatggaact cttctttcag acaaaatccc |
| 71521 | ttaaacataa tttcctgggc cctcaatgat ctggatgcta cctaagtgtc cagcctcaaa |
| 71581 | tgtcaatgct tttccccaca aacactactt accactaaga cccaaattct tttcacttct |
| 71641 | tgggttatgt caatatctgt cacctccagt attgcaatgc tgtactctgt gtttgaaaca |
| 71701 | tctccctaca catgatcccg tatctcgatg tatctagtta atgtctactc atccttaggc |
| 71761 | ctaaacataa atgtgacttc caggacattc bittatgtgc ttccacagca tccattgttg |
| 71821 | ctgtatctta gcatcattac cttatctgtc agattgtttt atctttgttt aattccctag |
| 71881 | gttttttttt tttttttttt tttagatgga gtctcactct tgttgcccag gtgggggagt |
| 71941 | gcagtggcag gaactcagct cactgtgacc tctgcctcct gggttcaagt gattatcttg |
| 72001 | cctcagcctc ccaagtagct gggattgcag gtgtgcacca ccacacctgg ctaatttttt |
| 72061 | gtatttgtag tagagatggg gtttcaccat gttagccagg ctggtctaga aactcctgac |
| 72121 | ctcaggtgat cctcccacct ctgcctccca aagtgctggg attacaggtg tgagccactg |
| 72181 | cacccagtct tattccatag gttctagact ccatgaaaac aaagattgcg tctgcttgtt |
| 72241 | acactgaaca atagcacagt agttggcaca tagcaaacac ttagaagtga ataaaatgaa |
| 72301 | tatttgaata aaaagaaaca aatttggggt caaaggtcaa attttgaata tgctgatttg |
| 72361 | aaagagtctg aatggtgttt aggtggagat accagaaggt ttgatgtaca attctaaaat |
| 72421 | tttagaacaa taactgggtc tttaaatata gatttgggag gaagcatcat tctgataaat |
| 72481 | gtcatttgaa gcctggtgtg cttattaatt cttagggtat tgatgtatca caagagaaga |
| 72541 | gcaaacatca gtaccaaggg aaagccaaca ttcatcatta gcbgtgccct gcatttccct |
| 72601 | ctttgatttt gtattatcat gcccttttcc tatccaaagg attcgcttat atttttgttt |
| 72661 | atagtataag gaattttttt ttttttgaaa cggagtctca ctcttgtcac ccaggctgga |
| 72721 | gtgcaatggt gtgatcttgg ctcactgcaa cctccacctc ctgggttcaa gcgattctcc |
| 72781 | tgcctcagcc tcccgagtag ctgggattac aggtgtccac caccacgccc agctgatttt |
| 72841 | gtattttaat agagataggt tttcaccacg ttggccaggc tggtctcgaa ctcctgactt |
| 72901 | caagtgatcc actcgcctcg gccacccaat gtgctgggat tacaggcatg agccaccgca |
| 72961 | ccojgcagat agttttaatt taaattcagt tagacaagtg catgtggtac acccattatg |
| 73021 | tgacgcttca tattatgtac taaaagcatg ctatgcacaa agtgtaggtg tacagtgatg |
| 73081 | gatagatcct gccctatgga actaacaaat tagcaagcga aatcaacatt tatcaagtaa |
| 73141 | ttaaaagtag tttaagtgtt gtgaaggaaa agtacaaagg cttatggaag tgcatatcca |
| 73201 | ggatgtttat aaaccaaggg aggtcgaggc ataaaatttc tgaaagcaag aatgcagtgt |
| 73261 | tcctacattt ttactaatat tccaagttct gaaaacgtgt acttaagtag cagcttttcc |
| 73321 | tgagtttatt caatttattg cattatcagt gtagtatgaa tgggagtgtg tgatgatttc |
| 73381 | cttcaaatat aaaaggaaat ttcttaatgc gttgttttct taaggtaaaa aaaaacaaaa |
| 73441 | aaaagacttt toittttgtt aaaagagttc caggcacatt ttgtagcaaa gccaccaatt |
| 73501 | ggcatgatgt aaattgtaag gacaatgtac cttaggtaat gttaaactca agttttttat |
| 73561 | agatacttca tagaaatgct tttttaaaat gggactcaat gaaggggagg ttatcagggc |
| 73621 | gtgtgaggaa gccagcbgtt cattgaaaag taaaagcata tgcactgtgc attccaaagt |
| 73681 | atactaatgg ataaaaactg cttcaaagtt ctccacacaa gtatctaagt ggctttgaaa |
| 73741 | agttaagtag attggatgtc ttttcaacct ctttcataaa catacacttt gagtatattt |
| 73801 | tcttatgaga agttgtatgt ttaagagata taaccaaaaa gtagaaaatg aattttgcag |
| 73861 | ggggtaaatt atttggtgat gaaagcagaa gcagaagaaa gtcatctgct tttgaggcac |
| 73921 | acatttggat taaaagttac ttgaatgtta gtatttacct tatatatttt actaaatcta |
| 73981 | ggaaagaata aagaaaaaca gctcaatctc tagagtcttc ctgaaaatgg tggggttaaa |
| 74041 | tcaagctcaa ggaactagaa gttctttgct gtgttcatta gtgccagact aaagaagcca |
| 74101 | gtttagttaa gaagctagct tgctatgagc taattctgat acatagaagc agcattattt |
| 74161 | taaattctac attgccatct taaaaacaag cagctacttc tttttcatta ttttataaag |
| 74221 | caataatatg agcaatcaat aatatgagca tgtatattaa caaagacatg ctcgattata |
| 74281 | tttaagttgg atgtttcata aaattttaca acaaaaaata tttaaaccta agtaaaatat |
| 74341 | gcaactatat aatgaaatta gaggtattat tttgcactgc agtcagagat atttcttcca |
| 74401 | ttaattaaac agaatactgc agcagtctgg aagattaatt tattacattc ctttgtattc |
| 74461 | agcaagtgga acgtgatttg tattgtatct ttagcaagtt aatatttggc aagcacagtt |
| 74521 | tttggaaatc aggttttatc ctgatgtgga aaaagaactc tatgccagta ccagttatca |
| 74581 | gaactttctt taagaataac atttgcctta agaacataat taatgcatct gtgaaaaata |
| 74641 | ccactttttt ccaacccaag gtacaatgag attcacgtat tttattcagt atttctaaaa |
| 74701 | ctcttcaatt gaggttttta ttttaattgt ggtactgtgt atacttttta tgcataacaa |
| 74761 | tatgttaaaa ttaattcagt accagattta aatctgttag gctatctgtt attccatttt |
| 74821 | ctttcccaaa cccaatttaa gttccacagg ccttttatag ctggccatgt ggttgcaatg |
| 74881 | ctaagtcaaa atgctaagac cttttcccat tttggggcat tatttcttta aattcgttgt |
| 74941 | agttttaaaa tctgatttta tagttattaa tcatattctt ctgggggtac atatatctgt |
| 75001 | acttaatgga ctaaagtttg gaatatttaa atttaaatta aatatggtag tgagatttgt |
| 75061 | tctgaagaga aactggttat atttaggata taatggtgtt ttaaaggaag gcattttttg |
| 75121 | tgtcatattt gtatccacac aaacattact tataatttag tactaaatta taactcaaga |
| 75181 | tcctctcagt aaggaagttc tacacctttt tcttgaatag ccagcaagat aagtacgatt |
| 75241 | aggaattgtt gctttatagc cgttgctatg ctgctgtaaa caacctattg tttcaaaatg |
| 75301 | tggctttatt tgaagtccat tctbittggt gagaattgac tattaaatat tcctattaaa |
| 75361 | gaatgccagt agattacaac tgggaacctc tcagttggaa acacttgcca tttcctcttg |
| 75421 | tttattatag ttttacagaa tatacagtca gatttttttt tcagtttcca accttgctta |
| 75481 | aatgcctttt tttatatgtc cttttgatct tctccaaaac caacaactgg tattatatgc |
| 75541 | ctaaagtata tgtcaggctt cataatgaat tgtattatta caactttttt tgtgtcctac |
| 75601 | attggacaca agttaatatt acttattggt tcagaacaat tcactttatt gtataaaact |
| 75661 | gttttagaaa aatcataatt ttcaaaatat caaattataa tagatacatt atcagttaaa |
| 75721 | tacatcatca atcagtattt actaaataaa catacttgga gctattagct agtgctgaat |
| 75781 | aatacataaa ttttatgtgc tatgaactta aatgtagaac agaaaatact attatataca |
| 75841 | caataaagca caaaatagtc tgacaatagt atcatgattc atattttctt aaaataatct |
| 75901 | acttactgtt tcaaacattg atatgtttgg atagatctaa actcttttga gaggaaattc |
| 75961 | tagttactat gttagttcag atcattatta atggcaagaa caaattgcat aagggaatta |
| 76021 | aactccagtg tttacggtaa gctatttggt atttcagaaa ttctaccata gtatctctct |
| 76081 | ctgtgtatat gtatgttctc attcaccctc accgtgaggt ctcttttttt ttctttttct |
| 76141 | tttttttttt tttttttttt tcggagaggg agtccacctc tgttggccag gctggagtgc |
| 76201 | aggtgcagtg gtgcaatctg ggctcactgc aacctctgcc tccojggttc aagaaactct |
| 76261 | cctgcctcag cctcccaagt agcbjggatt acaggcatcc gccacgccac ccagctaatt |
| 76321 | ttagtatttt tagtagaaac gggatttcac cabittgggc cggcbggtct tgaactcctg |
| 76381 | acctcaagtg atcctcccac ctcagcctcc caaagtgctg gggttacagg cgtgagtcac |
| 76441 | catgccccgc cctcattgtt aggtttctga tcagcataga ggcaaggtca ttactgatga |
| 76501 | ctgttatcct gagattctag caaggtgctt ggtatcatgt aggtactgaa taaatagtta |
| 76561 | ctgaaaacta aacaaggaaa gattttaaat aaaaatttta aaatttagaa tttctttaac |
| 76621 | aaaaatttaa ctaaatcttg gctgatgtga atgatgataa agaacaagag aaagaagagg |
| 76681 | aggaagagaa atccaaacat tacactgaaa tcaccattat tattatccct attttatagg |
| 76741 | taatgaaatg gaggcttggg gaagttaggt aatttttcca agtctcagaa gtagtaaatt |
| 76801 | gaagatccag gactctaatt actgtctgtt tctcaagact aagtaaactc ttgtttattc |
| 76861 | ttacatactt cgtggagtag ggaagttgcc agtgatttga aaatgttatg tatttatttt |
| 76921 | gggagggcgc catgcaagtt tataatgcac ttctaattat tattttttca gttcaaaaaa |
| 76981 | tgtatatttt ctaagcacat actatctagg aaaaaaagct tacggaaatt cttagtaacg |
| 77041 | tagggattaa atatagctat gtaaaaattc acaaagtggt ctgtaaacaa gaaaatagaa |
| 77101 | tttactatat ttattgaaaa ggcagtagaa actgtaatca tgtctaactg ataagagtaa |
| 77161 | gtcagtcaat atacacacat gagaccagca cagaaagaat tagaaaaatt aaagaggcct |
| 77221 | ctttagtatg tgacaagagc tggtataggt acaattatat cttaatacat acccagatgg |
| 77281 | catattccca gtagatatta ctaattttcc tgaagttgat agataaataa aactcaatta |
| 77341 | tcaagactct ttctactttt aatattatca aaggtatctt tctctggact cttaaaattt |
| 77401 | tttcacactg gtatcatcaa gtagcaggtc aaaatttagg gatagaggat gtgtttcaag |
| 77461 | agaagaacaa aattttttaa ctttaatttt tcaatctctg ttccttttga caaaattaac |
| 77521 | tgaattcagc ctgccaattg ctacttagac acaaaaaaga gaaatgagac attagttatt |
| 77581 | aagattttga ttttgattcc tttatgttgt gatttccttt atttccttta agtagttaaa |
| 77641 | ctgcaacaaa gatttttttc tctccaactg taaaaaagtc ttccatttta ccacttataa |
| 77701 | aagtcttcaa cttagaggaa aagacagttc tctaaacagt gatttggttt tagtgctgtt |
| 77761 | ttacctgtct ccttattctt ttagtcagtg tattcaaaaa cacatttctt gcagcctttg |
| 77821 | aaagcataga gccacactta gttttccagg aagatgcttt cttaatgtga attagctatt |
| 77881 | cttcatctca ccagaacatt cttcaaagct gctgttcctg aattcatgct tgatacccat |
| 77941 | gggtggatcc tgatttgtga acattatcta cctaggcatg gatgaggtct ctccatgatc |
| 78001 | actgatactg ctcacggcaa tctgtctacc atttggaaaa caggccgggg cagaatctgg |
| 78061 | aagggcagcc atcagtggga gcaggaatga tgctgccaag ggttaggggt gggaaaacat |
| 78121 | aaactcttct acaatagctg ttagttatct caaatagtgc cttaccttca ctatcaggct |
| 78181 | ctgtcctgcc tggtttcact ggagacaatg ccaaatcctg ctgtgcttac ttattacttt |
| 78241 | gtttttgcat tgctataaag gaatacctaa gactgggtag tttataaaga aaataggctt |
| 78301 | aattggctta tggttctgca ggctgtacag gaagcatgat gctggcattt gctcaccttc |
| 78361 | tggtgatgcc tcaggaagct tgcaatcttg gtggaagggg gcgcbggtgt gtcatatggc |
| 78421 | cagagtgtga gcaagagagg gaagggagag gtctcagact cctttaaaca accagctcta |
| 78481 | gcatgaacta acaggaaaat ctcacttctt accgaaggat ggtgctaagc cattcatgag |
| 78541 | ggatctggtc ccatgaccca ttcatctccc gccagctccc acctctaaca ttgggaatca |
| 78601 | catttcaaca tgagatttgg aggagacaca gatccaaacc atatcagatt acaaacactg |
| 78661 | tgcgtgtaag caaaattgtt tctgcbggtc tgagccactt gagccctttc ccactagtca |
| 78721 | catggagcac ctcttcttgt cccccgtctc cactgctgct tccttcctac agcccctgcc |
| 78781 | ctgccacagc ccttggaacc acatcgggtg ctggttatct cctgtcaatt cttgttacat |
| 78841 | ttgacttccc gaagacatgt ggccctgtga ccacacacac ctgtttgaag ctctcccctc |
| 78901 | tttctctggc acccgttggc ttgacagtct cccctcctga tgctccatca ttgttatgct |
| 78961 | tcttcccaga catattcact agttcctttg cctcttccca cctcatagat gttcctgtgc |
| 79021 | cccaggtttc tattcttaga cattttctct tttcatttag agtgctcatc aagtaatctc |
| 79081 | atcaatgtcc aagactccaa ttacagctta aatgttcatg acttttcaat ggctctccta |
| 79141 | aactctagac acacatatgt gactgcctac tagacctaaa ttcttaggta caccttccta |
| 79201 | ccatctacat tattgcagag cttaaataat caaattataa attatacaaa tataaaaaat |
| 79261 | aagtaatttt ataaatttat taaaaattgc tttttaaaac cactttgtat gtcttctgta |
| 79321 | gtgctagttc ttaatataga ttccatagat gtcttaaaag atagaaaaat tgtagcttat |
| 79381 | gaagacactg cagaattctc tcttaaaata ggtatatata gtgcaaagga ttactgccat |
| 79441 | tgcaatattt cacctaatca aagattttat ccaacgtgta taaacgggta tctttttaga |
| 79501 | taagaaactg gtcaaaatca taatagtggc atatgttgag gtcacatagt gaagttatct |
| 79561 | atgcagatca gacagtattg tggagaatac ctagaatctt gagaatttac acaaatagta |
| 79621 | ataacttaga tctgtatagc aaataatata catatcagat tcatttagtt tatgtaatct |
| 79681 | ttataatctg agaataatga gtcaagtgtt atccttagtt tactgattat caaagaaaaa |
| 79741 | tacagcttgg agaagtcaaa tgaattttcc aaatttatgc tacttataaa tgggagagct |
| 79801 | aataaaagcc acctgtctgg gtataaccct gtacctattt cttctaaagc atattgtttt |
| 79861 | ctaaagaaag tagttacatt ttaaatgaat atataaggaa tacatcttga atgaagacaa |
| 79921 | tatgcatttt ataatttaca tatttctgta tttcattatt cagaattcat ttaaaaagtc |
| 79981 | tccttgttgg catggacaaa attggcagct aatcctatcc cataaatatt ttcaatggtc |
| 80041 | ttcagctgat ggcgtgaaag ttcagcaaag gccaagtcac aatgctttga agtttgtcat |
| 80101 | cttttttatc actgctcaga ttgtcatgaa gacaccaagt aatacatgga tcaattatag |
| 80161 | ttaaatgact gcaagttgat cttatcaagc agttagtggg caagatttca cttttaccat |
| 80221 | tttttctggt aatgatttag agtgtaagaa cactaggaat ttaaatctac ttagttttac |
| 80281 | ttaatcaatg atgtatttgt ttatttaatt taaaacattg tttttggaga aggaagagaa |
| 80341 | cacaagaagt tcttgatcca taaataaggc gttcagaagg agaccagtta aactaaattt |
| 80401 | tgactgtaat tcagaattgt gtaaaatgcc ctactttatt ttaagctcat tattttcata |
| 80461 | attcctttac caagatagca gaaacatggc tttatctatt aaattataaa tgtagttttt |
| 80521 | tttgttttaa acataatcaa tttataagag atctaagaca tataaaacta cattgaggta |
| 80581 | gaaatttaat aaaaattaac tttacatcta tgtaacttac gtctatgtat tcagtttatt |
| 80641 | taaagtaggt cataaccaag tatttctggg ttgtgtggcc aatagctttg ctcctaaatc |
| 80701 | acaaagatgt ttctgtgtgt gacacttttt aaatccctgg aatattttaa atgttcattt |
| 80761 | caaataatct ataactttga aacagttctt ttttcttgaa cacattgttc tgtcacgtct |
| 80821 | aataggataa ccaattagtt ggttatccta attctaatat gcttcataac tagtgacata |
| 80881 | agataaacat atggagtatt tattactctg ataaaagata ctggttagga gcaagacagt |
| 80941 | ttttactttg taatcataaa aacatagcgt cctgcgtaca ctaggcagct acttatatag |
| 81001 | agctaatctc actctttttc ggactccaat atttaccata aaagtgcttt acagcatcta |
| 81061 | tataattttt tcttcttttt gtcccttctt tgtagttatt tgaaattaca gtgcctctct |
| 81121 | ctcaaggccc caaaccagta acaatcagtt ttgccaatca cacttcctgc cgatgcatgt |
| 81181 | ctaaactgga tgtttacaga caagttcatt ccattattag acgttccctg ccagcaacac |
| 81241 | taccacagtg agtatgaatt aaatctattt tttctgcatc ctctactaat attaaattat |
| 81301 | tttcaagtaa acataagtta aacaaataac ctctgtgaaa tttactgtaa tatactctgc |
| 81361 | ttcaaataca aaacaaagac aaaattttgt atagtataca atgaaaataa actgaaaatt |
| 81421 | aaaattcagt gggaagcgta ttttagaggc aaagtaattc acaatataaa agaagtatgt |
| 81481 | ctgtgaaata aaacacattc caccagtgtt cataaaatat cagtaaggta gcttaattga |
| 81541 | tttaacattc ctgaaatcag gtatgctaac tcataaatca tattaaagtt caaaacattt |
| 81601 | cagccttgat aagattttag gccaaaacaa aaagtaaata taaataattt tatatctatt |
| 81661 | agcattcagt tgctttcttc aatctcaaac ataaaaatat atatgbitgt gtgtgtatat |
| 81721 | gtgtatatat gtatctccat gtttcttatt tcagcaatat ttgaatagtg aaatagtgct |
| 81781 | taattacttg aaaaaaattt ctgttaatat catttcaaat tgacattcaa accattattt |
| 81841 | gttattcagg tttcctttcc atcctatttt tttggagggg caaatggcag catatacctc |
| 81901 | atgtgatcat ataggagaac ttatcacgtt ctcctgtatg atcatgttct gaccctgtga |
| 81961 | atagctgata atgaattaat atgcattact gcctaagatc tgagagtttt ctgccccaaa |
| 82021 | gtagggatgc aaccctttgt tccttaggat atccggtgca tatggatgaa acttacataa |
| 82081 | ctctagcatt tgtaacaaat gtataaataa attgatcaaa atacatatat attacaaaaa |
| 82141 | agaaaacctt acatttttaa aagactcaac aataaatgta atatgcaaac catggttttc |
| 82201 | cccactgaat cacgttgtac tactctgctt agtgtggcag atagggggac cccttgtttg |
| 82261 | tccagggaca gtttaggaaa acctggaaga tagcctaatt tgagtgacag agatctgtcc |
| 82321 | ccttttatgt gagaaagcaa agatcttaca gacatttgtg aataaatgta tgctaataca |
| 82381 | agcaagagac cactaccatc tgaactcaag aggatgccgt atgtgcaatt tcatccccgc |
| 82441 | aggctagaga gttctgttca ccttcctcag ccctgagcag aagccctcag tgactcctcc |
| 82501 | attctgagag ctttagccat tatctccata ctcatggctt ccaaaatgac atttgaagat |
| 82561 | ctaaccatat ttttgactta taatatttta tctaaatcta tcttctattg ctttgcttgt |
| 82621 | aattatatta gctgtaagta caaagagaaa gacttgttaa atcctttatt tctacattat |
| 82681 | tcatggcagt gttttctcat tttctttgag gccttgaaat cacagagttg tcattacttc |
| 82741 | ttcttcttct tctatcccat ctaaacacaa ttgattattt tggaggaggg ggagatatgt |
| 82801 | gttccaagct aaacttatat ttcatttcca tcactgccat tatcatccac ttcattatgc |
| 82861 | atgtctctga tctaatgcag tgatattaat actagttttc aagcatcaag tctttctcct |
| 82921 | gttgtataat gtcactgttt tatgaagtaa gctttaaaag gcatcgtttc attcatctca |
| 82981 | ccagaaacag tgactttcag ttgctcaaag aactgagcac aaatttctca ccaccctatt |
| 83041 | ctggtgtcag ccttcctttc ttgttttgtc ttccattatt cacccagtct gtgtttcacc |
| 83101 | aagtccgatt cccagaacgc caagctcatt tcctgctctg tatcttgttt ctattctggc |
| 83161 | cctggcttgt gttctgcctt tccaaagccc tctccatttc tcagagttca ggtaaaggtc |
| 83221 | tttctcctgc atttgtcttg tccgaatacc acggccaact ttgcttcttg aatacccata |
| 83281 | gcactcactg gattaacttc atcctcacat ggctttatca gggtctgcaa atgttgcatg |
| 83341 | tggaactgga ttatgagagt gtttccagta agtttatatt gtctgcaaca ttcagctaac |
| 83401 | aacactcaaa aatacttgag ccattcctgt gctctcagcc cctttacctg cattgtctga |
| 83461 | tttaaccctt aacaaaatca tctagaaggt cagcagcagc atttgcctta ttttacagat |
| 83521 | aaaggtggaa atcacactcc aaagcbgttg gctaaataat gtacataaat gcttgttaaa |
| 83581 | taaaatgact catttcctgg atgtatttta tagcatttgc catgttttca tcattactta |
| 83641 | ttcatataac tactatccta tacactgttt gaggtcaaga agagacagga agctacagca |
| 83701 | aaaagagcct gagtcttaga gtaagccata cctgattctg aattcagatt tgccactaat |
| 83761 | tcattctctg tgatcaggta ggtgttttat gtctctgaaa ctcagtttac ttatatataa |
| 83821 | actgtaaaga tgtaaagagg attgaatgaa gtactgaatg cttatgcaat gcttagcaga |
| 83881 | gtgtctggta tccagaaggt agtcattaaa gggcaaattt attattacca ttaatatcta |
| 83941 | agtctgattc atttcctcac ccatgtgttt ttcagtattt ttaaaaccac tatctacagt |
| 84001 | aagaaaaaca ttttatattc caaaccagga tgtgtggtat acacacacat agtgtgtctc |
| 84061 | tgtgtatata tatatgtcga tattagagaa ttttaaatta gctagataac ttcacatgta |
| 84121 | attaattatg tataagcata aaactaaaac aaattaccat ttaaaaatat tgtccttaat |
| 84181 | tatggtgctg ctcaagcttt ctcattatca caaccccaag gatcctttta taaacatttt |
| 84241 | ttccctaatc actttcccca taaaatttta atgtaacaaa tatactctgt ctttatacta |
| 84301 | tatgtatctc tgtgctttat acataaaaag gaagtgtaaa gtttactttt ttgttcaatg |
| 84361 | tagtgttaaa taacaaataa aatgtttaag ttaacattta aattaaaaat ttttaatctc |
| 84421 | ttaacagcta acataaattt ataattgtat ttgccaggaa acttttaagg taatctgttt |
| 84481 | acttatataa attgtaatat atcaaattac atatgcaaag taacaattga tatgaatata |
| 84541 | taatacaaat ataataattt ataaaaacat tcaaaatcac tatttttcca gcaaaagtaa |
| 84601 | aataattttt aaaataactt ctttaaaaat atttggagag cttaactatt aactttgctc |
| 84661 | agtatgagaa aacttttaac ttatatagat gataaatatt aatttagata ttgtcaactt |
| 84721 | tttcttttca tcatttgaca tgtaatggta tgactaacat ttgtagaatt aataataaaa |
| 84781 | tataaattat tattatttaa ttctcaaagc tcaagtcaca catgaaaaca ccaaggacca |
| 84841 | agcataagca atatccacag ggcaaccaat ggcagccaat aggcccacat aggtaccact |
| 84901 | cagtatatgt ttgaaatcac tcagtcaatt aagagaaaat cctccagtca tagccatcta |
| 84961 | gtcaccataa tttcatagct catatattgc aaacacttgg ggtgtgttcc gttgccaatg |
| 85021 | ctgttcatgt aatggccaat aaaaccaaat agaatttaag tattaaggaa tattattttc |
| 85081 | ttgggttttg gagagccaaa aatcattgag atattaagat tttagcccca tccaggcctc |
| 85141 | atacccaatt tttaccccct tagagatgat tttactccct ttatgaatat ttgccttatt |
| 85201 | attacgtgtg atatacccaa ataatttttc tattctgttt ttctttgtga aaatattggt |
| 85261 | ctcaaccctt tatgaattat ggcccacaat tcaataaaag ctacactagt gataatatat |
| 85321 | atatgatgga catgtgacta tgttataaaa ttctaacatt tgttaagatt taagaggaac |
| 85381 | aatcaccacc acggtggcta aaattaaaaa cactgacaaa aacaagtgtt agaatgtggt |
| 85441 | gcaaccaaaa ctcctatgtc accaatgggc attaaaattg ttcaatcatt tttcaaaaca |
| 85501 | gttgacagtt tcttatgaat ttaacatgta catatttcca cacaataact tgtgtacaaa |
| 85561 | tgttctttat tagtcaataa ctggaaacaa ctcaaatgcc catgaaagat gagtggatta |
| 85621 | acacatctta gtatatgcat acagtggaac attgctgagc aatgtaaaaa gaatgaacta |
| 85681 | ctgatacaca caagaacata aaaggatctc agagacatta agttgagtga aataagcttg |
| 85741 | gctaaaaaga gtacattctc tatgatttga tttacatgag gcccttaaaa gacgcaaatg |
| 85801 | tactctagag tgagagaacg caattcagtg tttgtttgag gatttggatc aggattgact |
| 85861 | gacaatgggc aagagagaca ttgtgaggtg ttagaaacat tctttatttt gattgtgaca |
| 85921 | gtagttaatg tggatacata aacttacgaa aagatgataa attcaaaata ggagcatgtt |
| 85981 | attgtatgca aattattccc caataaaatt gatttttgaa aagcctagaa aagaattcct |
| 86041 | ttattaagta caatttttta agtagcttag gtttcaaata gtagatttct cttttttttt |
| 86101 | tttttttttt tttttttttt gagacagatt ctcactctat cacccaggct ggagtacaat |
| 86161 | gacgtgatct cagctcactg caacctcctc ctcctgggtt caagcaattc tctgcctcat |
| 86221 | cctcccgagt agctgtgatt acagttgccc gccaccatgc ctggttaatt tttgtatttt |
| 86281 | tagtagagat ggggtttcac cattctggcc aggctggtct tgaactcctg accttgtgat |
| 86341 | ctacccgcct cggcctccca aagtgctggg attgcaggca tgagccacca cgcccagcca |
| 86401 | aaatagtaga ttttttaaaa cctttacaat aacattttaa gtcaaatctt tgattaggga |
| 86461 | gggagatttc ccacagtcat ccaaaaacta tacgttgagt cactaagata aagaatgctt |
| 86521 | ctagatccta ttagagaaaa caaagatgcg cttggctggc tctgaactta gagcacttat |
| 86581 | agtcaagtaa gaacaacaca tattatagtt acattattat tgttttattt tactaaatat |
| 86641 | tacaaagcac caagcatctg ccacacattg tctcatgttc atacaaatac tgccggtggc |
| 86701 | tccattcgta atacacatca catagtacaa ggctgaagag tgtggatgct gtagggagct |
| 86761 | tccagttttg agaatttagt ctctgatttc tctttgagtt tgagtaggtt ctgtaaactt |
| 86821 | tcttgtgaca catttttttt aatctacaaa agagcacata agtaagaatg ttacatataa |
| 86881 | catcatacag aatatttata agctaatctt cactgaaatc aatctgttca atagcattat |
| 86941 | accatatttg acataccata gccatgttaa tctgatattg tagaatagca tagtataata |
| 87001 | ataataactc ctaactcaag gabittgtga tctttataac cagcaatcca tgttaaatat |
| 87061 | tagcacagtg cctaaaacat attaagcatt caataaatga tcgctactat ttttactaac |
| 87121 | atcctacaga tttggaaatt gagtcttaga aatgttaatg tgtaaaatgc taaagagcca |
| 87181 | aaaaaactgc caggaaaata taaaaattaa aatcatttta tttctgaaac ccatgtgttc |
| 87241 | cccccaatat cttctaacat ttctagtatt tacagaaaaa ctttcaagtc tcaatatcag |
| 87301 | aaagtttcat aaaagccaga ggaagtagca attctcttta gcagacagag ttagatacca |
| 87361 | attttcatat tggtgttctc acagattatt ttttccaatt atttcctctc atattttctt |
| 87421 | caaatttaac tccatgtatt tccatgggcc ctttagtaga gactttcttt tcatggaggg |
| 87481 | agtcatttgt gattaagtag cgatccccta gattttcctc cttggtagta gtgtttgttt |
| 87541 | attcattcat gagacattta tgtcaggtct tctatctcta taatactgta ttaggaaatg |
| 87601 | tggggtattt ttaaatgaat aatataattc tttagctaaa ataactcatg tactagagta |
| 87661 | caaacaaaca atatttctag tatagtaagt gtctaaaaca ctatgctgta cttcattcac |
| 87721 | tcattcaaca aatagcattt gtgtttctgc tagatactaa taaggtggtt agaccacaga |
| 87781 | gatagaaaga tacatagaat tgttactcca gaagaaattc cattcttgaa ccagacatgt |
| 87841 | aaacatatga ctgaaatcag tgaaatcagt gtggtgagga agcatcttgg agacatggct |
| 87901 | acagtagaga ggggatacat gagtctggag aggtcaacag aatcacatat gcccagccaa |
| 87961 | ggatgggctg tttcttaaag tctcttgaga actactaaaa gtatatatgc agagatgtgg |
| 88021 | tatctgcagt tttgtattat agaaagaaca gcttggtggc aagggagtgt gtggattaga |
| 88081 | ggaagcaaaa cgggaggtgg aaaaattcgg gagtaatcat ttgaaataat ctgagtgaaa |
| 88141 | aataataaag atggtggcca cagcagtgga tatggggata gaaagaaaag aggtggaaaa |
| 88201 | gacgtttgaa atacaagaaa gcatatgact tgggaaccgc atatggggga tggtaaagtg |
| 88261 | ggagaagttg aggatgaaac tgtatggatg acattcaaaa gacaaggagc acctgttcat |
| 88321 | agtaggaact tacctgaaaa ggagaactga gtttttaaca catgttagct tgagtgcttg |
| 88381 | taatatttgt aagacatcag agtggtagag taattggaag agatgacctc aaccaaaaga |
| 88441 | aatatagcaa gtaagaagac aagatgagcc acagatgaaa ctgtggggaa atcaatattt |
| 88501 | aagaaacagt tatctgtaga gcagacacaa taatctggag aaacagtaat ctgtacggga |
| 88561 | gactgagaag aagcaatcag aacaatcgga ataacaacag agagagagac cacaaaagcc |
| 88621 | agtggagaca tgagttcaag gaaggaggaa gtggtaagag gtgccaaata ttagaaaggt |
| 88681 | tcaagaataa ctgggactta ctctgttagc tgaacagcat gaaatagaga tgtttattta |
| 88741 | ctaataactt accatactct gggcacagag agggagaatg tgttttgaga aagggggaga |
| 88801 | gaaagcaaga cagtgaaaga aattcacatt tacacatgag aaactgaatc agaaagggta |
| 88861 | agtaaatttc cccaatgtca cacaactaga aaagtagcaa agcagagatt caaactaaat |
| 88921 | ttgtttggat ataatccatt tcttttgagg tgtggctatg actgcatttt tattgttttt |
| 88981 | tagttgatac ataattgtac atatttatgg ggtatatgtg atattttgat acatacatat |
| 89041 | aatgcgtaat ggtcagctca ggatattttg gatatccatc acctcaaaca tttatcattt |
| 89101 | cttbitgttg taaacatttt aaatcttcta gctattttga aatatataat aaaatattat |
| 89161 | taactattgt taccctactg tgcbgtggaa tactagaact tgtttcttct atataactgt |
| 89221 | actttggaat gcattaccaa cctctcttca ttccccccac aactgaccca cacttcccag |
| 89281 | actctagtaa ccatcattct actctctact tccgtgagat tcactttttg gctctcataa |
| 89341 | atgagtgaga acatgagata bitgtctttc tgtgcccagc ttatttcact taacataata |
| 89401 | gcctccagtt ccctccatgt tgatgcaaat gacaggattg cattcttttt atggctaaat |
| 89461 | agtactccgt tgtgtatata taacagattt tctttatttt taacttctat aaattaaaaa |
| 89521 | atatcaaaca tcaaggacaa aacaaaaaca aaacaaaaaa gttcccttct accccaattt |
| 89581 | gaaatgtaac aactattttt ttgtatttac taaagataca acccaaatcc ccagtcatca |
| 89641 | ttcttcactc ttcattccca gaagtaacca ccactcaaaa attgtattat tctcactctt |
| 89701 | ctgtattttt atggcatctg gatttattaa cataccagaa gatattatac agtgcagctt |
| 89761 | tgagtgtttt gtattttaag ttaaaatgtt ggcattactt tttttctctt gacattatgt |
| 89821 | tttacagata tagtcatatt gctatctgta cacctggtgt attcatttaa taactatata |
| 89881 | gcattccatt aaataaacac aagtttttat gtttttttta ttctgtgacg atgtactata |
| 89941 | gggaagtcaa tatacactac agattagcaa gagagtacaa tcaaagagga gagagtgatg |
| 90001 | tcaaatagac aaggaccttg aagttaatag tgctaaattt attagttatt tggttagcat |
| 90061 | ggacaatgtc cctaaagatg taataaaata acacgttctg tgcacagtta ataaaccagg |
| 90121 | ttgatttcag tcttaagctg tgaaagcaat tggatattaa atgaaaacac aatactgtga |
| 90181 | ttctaaagct ttgagcagca gcctagggaa tttcacctat tctgtagagg tattagggga |
| 90241 | gcagaggttg tttctgggta gaggaatgat tgacaagaca abittcagca tagccaagaa |
| 90301 | atcttgaatt atttttaatg gataataaat atagaagata ggtattattt caatttggaa |
| 90361 | gacagtcgcc tcgtggtgaa aaagagaaaa gggatgaaaa aagatgagga aaaaaaatgg |
| 90421 | ataaatctag cctgaaggct aatttgcata tttatttatt tgctgtcttt tatttctttt |
| 90481 | attaaaatgt tgacattgtt tactcagtaa aacttatttg cttgcaagaa gttacactta |
| 90541 | ctagggttta tttaagaaga ttttattctt atgagaacat gtcttatttc caaggaatag |
| 90601 | agtaagtaga tttatagcca aggaaacatt tgatttggaa cttgaggtct ccgtgttttt |
| 90661 | ggtotattaa tatttaacag atcctcagtc caaatgccta gatgottaaa tatccataag |
| 90721 | ttgtactttt agccataatg cttcactctt tcctttttaa tttgaaggtt atacattatt |
| 90781 | gctttaccaa aagacaatga agcaaaatag cctaataaca taaatttaat aaactgtatc |
| 90841 | agagcagaaa tggagagata gcttagcttc cagttaattg gaccacagtc tcgtactaga |
| 90901 | tcatcagaaa ctactgtttt tcagcatttt aaaacatgca tctttagtct cccctgaaat |
| 90961 | tatcctcttt gccagttcaa aatctttatg tccatatacg gttatttttg aaatcgtaag |
| 91021 | tcatatgtct gttcatcccc tttcctataa agctcattat ctaaacattg tctcacactt |
| 91081 | caaaggatca cctgttgatg tgaatggatt taccagtgga actcaggcaa gagattccag |
| 91141 | gctgtgagcg ttactgtttt tgtaaaggga aggtaacttt tatatatagt ttgcctgcct |
| 91201 | ttggacagtc ttgtttctac cttctaaaat acgataattg aaagcacagc gttcctatgg |
| 91261 | cttgctgata gattgtgaga aataacagca aaatatatac agctgaggag attgcatcaa |
| 91321 | tacatctctg caccgaaatt atggcagtga tttgcctaga attgggggct attagatcaa |
| 91381 | agattgtcct aaagtttaaa gttacattca accaaacaca tggacaattt ttgttaatat |
| 91441 | catattagac cagatttacc agttgttttc taaaatttaa tatacatttt ttaaccattt |
| 91501 | taggttacag aaaaagcaga aagtatagag agttttcata taacatcctc tttcacgagt |
| 91561 | ttcctctagt tttaacatct tgcattagca tggtacattt gtaacagttg aggagccgat |
| 91621 | actgacatgt gatcgttaac taaaatccat agcttacatt agggttccct cttttttggg |
| 91681 | tgtaagttct gtggattttg acaaaggtat aaatgatgtg tcaatcatta cagtatcatg |
| 91741 | cagaatactt ttactgccct aaaagtctcc gtgttccgcc tgttcatttc tcctcccctg |
| 91801 | tattcctggc atcaaccagc cactttgatg tcatatcaac tcttataccc tcataacgtg |
| 91861 | ttttttattg tcatcattgt tttgtttttg tttttctgtt aatgacgtat ggctgtgaca |
| 91921 | aggtaatgag accagojgct gttgttggag gacctttttt cttaattcct ggttacttat |
| 91981 | ccttgaatta tgtgacaagc tgataaatcc aataaccatg gattttccat tgtttcaacc |
| 92041 | tgccaatact tctctcaagc ttgtatatcc aagaaaatat aaaaaatagt agagcagaaa |
| 92101 | attacaagct tccatactta tcctactctc cctgtgtccc catccccact tctggtggct |
| 92161 | ttccaactca gttaagtcct gtacttagcc agttggtgac cacacaatgt gacatgtttt |
| 92221 | atatccaagt cagtctcaga ctaggtgagt tttttatgtg gcagattact gcttagttta |
| 92281 | tactcagaca aaaaggaaaa aattaaacat ataaacaccc tttttttttt ttcgagagtc |
| 92341 | tcatgcbgtt gcccaggcta gagtgcagtg gcgtgatctc agctcactgc atcctctgcc |
| 92401 | tccojggttc cagcgattct cctgcctcag cctcctgagt aactgggatt acaagcacgt |
| 92461 | gccaccacac ctggctaatt tgtatatttt tagtagagat ggggtttcgc catattggcc |
| 92521 | aggcttgtct caaactcctg acctcaggtg atctgcccat ctcgacctcc caaaatgctg |
| 92581 | ggattacagg cgtaagccac cgtgcccagc ctaaacccat gttttaaatt aatgcaatga |
| 92641 | ttgtattagc ctttcaaaca ttaagactgg caaaattgtt acaactatgg agttttcatt |
| 92701 | gattcatcct actcaccatc ttttccttta atatctgaac aaaccccaac tctgttcact |
| 92761 | gttctccctg tgagtagact aatggaacaa gagcaaacag taacacaaac agacgctaac |
| 92821 | acaagttcat gcagaatcaa taaaaatcag tacaacagaa gataagtcta tgtgtttttg |
| 92881 | atacatacga ttaagcatgt gcctttttaa caatttatat aaaacctaaa atatatgtat |
| 92941 | ttctgatttt tacctgtagt actgaagaag ttacttaata acgttgaata taaaggccac |
| 93001 | ttttacttaa ccaccttcca ttcactatta actgcbgttt ccaaagtgta agcaaatcag |
| 93061 | tctctgtgta catagtcaag tgtatacaag cgtcaggcgt aacaaactca agatgaacat |
| 93121 | gacagttcaa agatatttgg gacaaaattg ttgaagcatt tttacccagg gctctgtagc |
| 93181 | tctgacaatg aaagaatata gttgctcttc cagctgctat tcagacagaa agcttgggca |
| 93241 | agaagggtct gtatctatgt tcttcataat acaattacaa gtttgaactt cagataacat |
| 93301 | cagcagttgg catgtggaaa accaaaaccc tattttggta tttatcaaga ttgttaatgg |
| 93361 | agtcaggttt cccttatttg tttctttaat ggggtacaga acatcctgtt ggataacccg |
| 93421 | ctgagtgaca tgacgatgct ctgaaggaat gcatgagagc ttgtggtacc tgccttgaaa |
| 93481 | catgggtttc actaatgctg gtggctcaca cttcccattg aacaagacta gagataggaa |
| 93541 | ggctatttga gggacacagc tatggaacca taggtgccac atggtaagcc aaatttttat |
| 93601 | ttgttgtgtt gttgtgaaaa accttattaa aagagcttcc aatgagagta cttgattaat |
| 93661 | aacacagttc gtatctatag aaataatttg cttttcaaga aaatcatcat gtgctacagt |
| 93721 | tgaattgaca ttaatgttat cattcatttt gaatgatctg tgaagtattt taagagagat |
| 93781 | ctggggaagt aaatcaataa gtagtttaac ataaatcaag gtgcagttaa tttttttcaa |
| 93841 | ttagaaatat attacaaaga ttctgtcatt tccaacaacg tgaaataacc tggaggaaat |
| 93901 | tgtgctgagt gaaataagcc agacacagaa agatgcatat tactgatctc ccttatatat |
| 93961 | ggaatctaga aaagtagaat tcatagatac agaatagaag agtggtcacc acggtcttgg |
| 94021 | gggtggggga catggggaaa tgttagagtg tgacaacttg cagttacaag gtgaatatgc |
| 94081 | tctggagacc taacgtatag tacagcatag ttactctact caatagtatt ctcactgcac |
| 94141 | cccccaactc ccccacacac acagtaactc tttgaggtgt ggatatgtta attagcttga |
| 94201 | ttttggtaat catttcacag tgtatacata taccaaaaca tcactttgta taccttatat |
| 94261 | atgtacaata tttatcaatc atacttcaat aaagctggaa aaatgcatga atattatata |
| 94321 | tatgtatatg tatatacaaa tgtataagag attatagcag attatagctc tttgaaaaag |
| 94381 | aataacattt cagcccagtt ctgactaaga ccaaacaaaa ggtgatagca tgttttagtt |
| 94441 | ccttaaatgt ggatttgagg agtcaagaaa tctccaagtg taggaaaacc tccgtggcaa |
| 94501 | agagtgtaga atatgagaat cacaaaaata gcatgcacaa aatagcctgc tgtgatgttg |
| 94561 | aaagtataag agctagtaat tattattatt attattattt ttatttttat tattatactt |
| 94621 | taagttttag ggtacatgtg cacattgtgc aggttagtta catatgtata catgtgccat |
| 94681 | gcbggtgtgc tgcacccact aacttgtcat ctaccattag gtatatctcc caatgctatc |
| 94741 | cctccccctc cctccacccc acaacagtcc ccagagtgtg atgttcccct tcctgtgtcc |
| 94801 | atgtgatctc attgttcaat tcccacctat gagtgagaat atgoggtgtt tggttttttg |
| 94861 | ttcttgtgat agtttactga gaatgatgac ttccaatttc atccatgtcc ctacaaagga |
| 94921 | catgaactca tcatttttta tggctgcata gtattccatg gtgtatatgt gccacatttt |
| 94981 | cttaatccag tctatcattg ttggacattt gggttggttc caagtctttg ctatcgtgaa |
| 95041 | taatgccgca ataaacatac gtgtgcatgt gtctttatag cagcatgatt tatagttctt |
| 95101 | tgggtatata cccagtaatg ggatggctgg gtcaaatggt atttccagtt ctagatccct |
| 95161 | gaggaatcgc cacactgact tccacaatgg ttgaactagt ttacagtccc accaacagtg |
| 95221 | taaaagtgtt cctatttctc cacatcctct ccagcacctg tbitttcctg acttttgaat |
| 95281 | gattgccatt ctacgtggtg tgagatggta tctcattgta gttttgattt gcatttctct |
| 95341 | gatgaccagt gatggtgagc attttttcat gtgttttttg gctgcataaa tgtcttcttt |
| 95401 | tgagaagtgt ctgttcatgt ccttcgccca ctttttgatg gggttgtttg tttttttctt |
| 95461 | gtaaatttgt ttgagttcat tgtagattct ggatattagc cctttgtcag atgagtaggt |
| 95521 | tgtgaaaatt ttctcccatt ttgtaggttg cctgttcact ctgatggtag tttcttttgc |
| 95581 | tgtgcagaag ctctttagtt taattagatc ccatttatca attttgtctt ttgttgccat |
| 95641 | tgcttttggt gttttagacg tgaagtcctt gcccatgcct atgtcctgaa tggtaatgcc |
| 95701 | taggttttct tctagggttt ttatggtttt aggtctaacg tttaagtctt taatccatct |
| 95761 | tgaactgatt tttgtataag gtgtaaggaa gggatccagt ttcagctttc tacatatggc |
| 95821 | tagccagttt tcccagcacc atttattaaa tagggcatcc tttccccatt gcttgttttt |
| 95881 | ctcaggtttg tcaaagatca gattgttgta gatatgcggc gttatttctg agggctctgt |
| 95941 | tctgttccat tgatctatat ctctgttttg aaccagtacc atgcbgtttt ggttactgta |
| 96001 | gccttgtggt atagtttgaa gtcaggtagg gtgatgcctc cagctttgtt cttttggctt |
| 96061 | agggttgact tggtgatgca ggctcttttt tggttccata tgaactttaa agtagttttt |
| 96121 | tccagttctg tgaagaaagt cattggtagc ttgatgggga tggcattgaa tctgtaaatt |
| 96181 | accttgggca gtatggccat tttcacgata ttgattcttc ctacccatga gcatggaatg |
| 96241 | ttcttccatt tgtttgtatc ctcttttatt tccttgagca gtggtttgta gttctccttg |
| 96301 | aagaggtcct tcacatccct tgtaagttgg attcctaggt attttattct ctttgaagca |
| 96361 | attgtgaatg ggagttcact catgatttgg ctctctgttt gtctgttgtt ggtgtataag |
| 96421 | agtgcttgtg atttttgtac attgattttg tatctggaga ctttgctgaa gttgcttatc |
| 96481 | agcttaagga gatttttggc tgagacaatg gggttttcta gatatacaat catgttgtct |
| 96541 | gcaaacaggg acaatttgac ttcctctttt cctaattgaa taccctttat ttccttctcc |
| 96601 | tgcctaattg ccctggccag aacttccaac actatgttga atagtagtgg tgagagaggg |
| 96661 | catccctgtc ttgtgccagt tttcaaaggg aatgcttcca gtttttgccc attcagtatg |
| 96721 | atattggctg tgggtttgtc atagatagct cttattattt tgaaatatgt cccatcaata |
| 96781 | cctaatttat tgagagtttt tagcatgaag ggttgttgaa ttttgtcaaa ggctttttct |
| 96841 | gcatctattg agataatcat gtggtttttg tctttggctc tgtttatatg ctggattaca |
| 96901 | tttatttatt gatttgcata tattgaacca gtcttgcatc ccagggatga agcccgcttg |
| 96961 | atcatggtgg ataagctttt tgatgtgctg ctggattcgt tttgccagta ttttattgag |
| 97021 | gatttttgca tcaatgttca tcaaggatat tggtctaaaa ttctcttttt ttgttgtgtc |
| 97081 | tctgcctggc tttcatatca gaatgatgct ggcctcataa aatgagttag ggaggattcc |
| 97141 | ctctttttct attgattgga atagtttcag aaggaatggt accagttcct ccttgtacct |
| 97201 | ctggtagaat tgggcbgtga atccgtctgg tcctggactc tttttggttg gtaagctatt |
| 97261 | gattattgcc acaatttcag atcctgttat tggtctattc agagattcaa cttcttcctg |
| 97321 | gtttagtctt gggagagtgt abitgtcgag gaatttattc atttcttcta gattttctag |
| 97381 | tttatttgca tagaggtgtt tgtagtattc tctgatggta gtttgtattt ctgtgggatc |
| 97441 | ggtggtgata tcccctttat cattttttat tgcgtctatt tgattcttct ctcttttttt |
| 97501 | ctttattagt cttgctagca gtotatcaat tttgttgatc ccttcaaaaa accagctcct |
| 97561 | ggattcatta atttttgaag ggttttttgt gtctctattt ccttcagttc tgctctgatt |
| 97621 | ttagttattt cttgccttct gctagctttt gaatgtgttt gctcttgctt ttctagttct |
| 97681 | tttaattgtg abittagggt gtcaattttg gatctttcct gctttctctt gtgggcattt |
| 97741 | agtgctataa atttccctct acacactgct ttgaatgcgt cccagagatt ctggtatgtt |
| 97801 | gtgtctttgt tctoittggt ttcaaagaac atctttattt ctgccttcat ttcgttacgt |
| 97861 | acccagtagt cattcaggag caggttgttc agtttccatg tagttgagca gttttgagtg |
| 97921 | agattcttaa tcctgagttc tagtttgatt gcactgtggt ctgagagata gtttgttata |
| 97981 | atttctgttc ttttacattt gctgaggaga gctttacttc caagtatgtg gtcaattttg |
| 98041 | gaataggtgt ggtgtggtgc tgaaaaaaat atatattcca ttgatttggg gtggagagtt |
| 98101 | ctgtagatgt ctattaggtc cgcttggtgc agaactgagt tcaattcctg cgtatccttg |
| 98161 | ttgactttct gtctcgttga tctgtctaat gttgacagtg ggtgttaaag tctcccatta |
| 98221 | ttaatgtgtg ggagtctaag tctctttgta ggtcactcag gacttgcttt atgaatcttg |
| 98281 | gtgctcctgt attgggtgca tatatattta ggatagttag ctcttcttgt tgaattgatc |
| 98341 | cctttacgat tatgtaatgg ccttttttgt ctcttttgat cttbittggt ttaaagtctg |
| 98401 | ttttatcaga gactaggatt acaacccctg cctttttttg ttttccattt gcttggtaga |
| 98461 | tcttcctcca tccttttatt ttgagcctat gtgtgtctct gcacgtgaga tgggtttcct |
| 98521 | gaatacagca cactgatggg tcttgactct ttatccaatt tgccagtctg tgtcttttaa |
| 98581 | ttggagcatt tagtccattg acatttaaag ttaatattgt tatgtgtgaa tttgatcctg |
| 98641 | tcattatgat gttagctggt tattttgctc attagttgat gcagtttctt cctagtctcg |
| 98701 | atggtcttta cattttggca tgattttgca gcggctggta ccagttgttc ctttccatat |
| 98761 | ttagcgcttc cttcaggagc tcttttaggg caggcctggt ggtgacaaaa tctctcagca |
| 98821 | tttgcttgtc tgtaaagtat tttatttctc cttcacttat atgaagctta ttttggctgg |
| 98881 | atatgaaatt ctgggttgaa aattcttttc tttaagaatg ttgaatattg gcccccactc |
| 98941 | tcttctggct tgtagggttt ctgccgagag atcctctgtt agtctgatgg gcttcccttt |
| 99001 | gagggtaacc agacctttct ctctggctgc ccttaacatt ttttccttca tttcaacttt |
| 99061 | ggtgaatctg acagttatgt gtcttggagt tgctcttctc gaggagtatc tttgtggcat |
| 99121 | tctctgtatt tcctgaatct gaacgttggc ctgccttgct agattgggga agttctcctg |
| 99181 | gataatatcc tgcagagtgt tttccaactt ggttccattc tccccatcac tttcaggtac |
| 99241 | accaatcaga catagatttg gtcttttcac atagtcccat atttcttgga ggctttgctc |
| 99301 | atttcttttt attctttttt ctctaaactt cccttctcgc ttcatttcat tcatttcatc |
| 99361 | ttccattgct gatactcttt cttccagttg attgcatcgg ctcctgaggc ttctgcattc |
| 99421 | ttcacgtagt tctcgagcct tggttttcag ctccatcagc tcctttaagc acttctctgt |
| 99481 | attggttatt ctagttatac attcttctaa atttttttca aagttttcaa cttctttgcc |
| 99541 | tttggtttga atgtcctcct gtagctcaga gtaatttgat cgtctgaagc cttcttctcg |
| 99601 | cagctcgtca aagttattct ccatccagct ttgttccatt gctggtaagg aactgtgttt |
| 99661 | ctttggagga ggagaggcac tctgcttttt agactttcca gtttttctgt tctgtttttt |
| 99721 | ccccatcttt gtggttttat cgacttttgg tctttgatga tggtgatgta cagatgggtt |
| 99781 | ttoggtgtgg atgtoctttc bitttgttag ttttccttct aacagacagg accctcagct |
| 99841 | gcaggtctgt tggaataccc tgcbgtgtga ggtgtcagtg tgcccctgct ggggggtgcc |
| 99901 | tcccagttag gctgctcggg ggtcaggggt cagggaccca cttgaggaag cagtctgccc |
| 99961 | gttctcagat ctccagctgc gtgctgggag aaccactgct ctcttcaaag ctgtcagaca |
| 100021 | gggacattta agtctgcaga agttacttct gtctttttgt ttgtctgtgc cctgccccca |
| 100081 | gaggtggagc ctacagaggc aggcaggcct ccttgagctg tggtgggctc cacccagttc |
| 100141 | gagcttcccg gctgctttgt ttacctaatc aagcctgggc aatggcgggc gtccctcccc |
| 100201 | cagcctcatt gccgccttgc agtttgatct cagactgctg tgctagcaat cagcgagact |
| 100261 | ccgtgggcgt aggaccctcg agccaggtgc gggtataatc tcgtggtgtg ccgttttttg |
| 100321 | tttttttttt gtttgtttgt ttgtttgttt ttgagacgga gtctcgctct gtcgcccagg |
| 100381 | ctggagtgca gtggcgggat ctojgctcac tgcaagctcc gcctgccggg ttcacgccat |
| 100441 | tctcctgcct cagcctccca agtagcbggg actacaggcg cccgccacta cgcccggcta |
| 100501 | attttttgta tttttagtag agacggggtt tcaccgtttt agccgggatg gtctcgatct |
| 100561 | cctgacctcg tgatccgccc gcctcggcct cccaaagtgc tgggattaca ggcgtgagcc |
| 100621 | accgcgccgg gccggtgcgc cgttttttaa gcccttcgga aaagcgcagt attcgggtgg |
| 100681 | gagtgacccg attttccagg tgctgcccct cacccctttc tttgactagg aaagggaact |
| 100741 | ccctgaccac ttgtgcttcc caagtgaggc aatgcctcgc cctgcttcgg ctcgcgcacg |
| 100801 | gtgcgtgcac ccactgacct gcgcccactg tctggcactc cctagtgaga tgaacccggt |
| 100861 | acctcagatg gaaatgcaga aatcacccgt cttctgcgtt gctcaggctg ggagctgtag |
| 100921 | accggagctg ttcctatttg gccatcttgg ctcctcccca agagctggta attattaccc |
| 100981 | aacgtttaga atataatgta acagaacgta tttggaatac atttatcttt taatatttca |
| 101041 | aaggagagat aaatgcaaac taagatttta tttatcatta aattcattgt tctttagttt |
| 101101 | gaaaaacaca tatactcata tttttgtcac gtgattttaa ccagcataaa gcaatagctg |
| 101161 | ccacaccaaa gtcaagtgaa gctgtgaaaa taattaatga aacaattctg ataaaagcat |
| 101221 | ctttatcctt catacaggtt ataggcagga ttgtggttgt gcctgtgtgt gtgtgtggtg |
| 101281 | gggtoggtgg ggggatagga aagttagttt attcacatga ttctcaacat agacatacct |
| 101341 | tgcaaataat tgtgcaattc cctgctttgc tttgcagaca gaatgtcttt gaatcaatgt |
| 101401 | cttttataaa tacactatac aaaaaacata attataaacc tttaaagtat ttatgcatac |
| 101461 | tttaatattc agaactagaa gagaaatttc tgtaaggaga aatgctttgg agattaaaac |
| 101521 | gtaaaggaga taaccaaatt ttactaaacc tctatccatc atgaccacat ttcctttcat |
| 101581 | tctaaaataa aaatattttg gaaatgttga ccccctgccc accatttttg cctgagaagt |
| 101641 | tttaaatgcg aagaacttgc tcctcacttt tgaagcacgg tgctttcttt aggaaccaat |
| 101701 | ctgtatcaca tgcattctat tggtttgtat tttattacat atcattctat tacattttaa |
| 101761 | aatgcatttc tgagcccatg tcattgattt catgattcat taatgggttg caagcagcag |
| 101821 | ctggaataac actggcttag gtaatggcag tataaatcac abittggtaa aatgaaagat |
| 101881 | gcctttctac caacacgtta taatggccac tcgatgccaa catggcagtg tgttacttaa |
| 101941 | accatatatc aatcaacaca gaagcgacat aaccaaaatg tttttaccat catttataga |
| 102001 | agattatgag tttcatgcag ccaggattaa ttgcattgct attatagaac ttaatataag |
| 102061 | ttgtatcata tttgatggaa ggatgctaac tatcaagagg atataaattt taaaaacaat |
| 102121 | gatactgttt tggataagtc ctgactgtct gcattttatc tagaacaata tatgttcatg |
| 102181 | attaacctag aaaataatga caaaaatgcg caatgagtag agacttgccc ttggaattcc |
| 102241 | taaaattatt aggaaggtac aatgttctcc aagaaatatt ggtaagtgaa aaaaacaagg |
| 102301 | tttacaataa tgtgtaacgg atgctatctg tgtaacaaaa tgtgaaaaaa catatatgca |
| 102361 | attttttggc aagaagatac cctgaaaaca taaaccaaaa agccaaaaaa aaaaaaagaa |
| 102421 | acttgctgat gatacagaac ttctctgact gtacattttt ctcatagtct taactctgat |
| 102481 | aattacatac atgtttgaca ttctcaaaag taagaacata gaagtacata atatatgcag |
| 102541 | gataaagcaa ataacagttt atgtgaagat tgttagaagc caaggttttc atggaagtta |
| 102601 | ttagtatgaa gtcaaagtag tacattaaaa tccagtaatg ttaattggat ttggaagtaa |
| 102661 | cagtatgaaa ttttttctct aaaaatgtgt gcaggaatct cttgcttaag atcagtaatc |
| 102721 | tattcccaaa aatagatttt gagagcacat ttctgttcta aatgggaaca attatagtga |
| 102781 | atggaaggta aaccaaaaca tagctatttc ctacaaaata attgatacct aataatgcct |
| 102841 | gtagaaaagc atagaaaagt gacaaactct cttgataaaa tctaaaatgt atattgtttc |
| 102901 | ctaatcagca attattgatg ttgctaacat acagttgctt tgtatctagt tacatggacc |
| 102961 | gtgtatatgc tgtatgagaa attgtcactt tgtggtatag catagcaaat aacacagcaa |
| 103021 | tcaaaaagtt aaaagtcaaa attaaaataa ataaaagacc cataatagac tgtttttcac |
| 103081 | cttgctaaag gagaaatgac tcttgaagaa actcaagaag ggacaaagaa aggtcaaatc |
| 103141 | agtcattgct atttgcagaa ggtgatgggt ggatggagga caagggggca atcttcgaca |
| 103201 | aactgtcata tgaagaggaa tgaagagacc ttccccatgt ctgtgtctag tagatgctgt |
| 103261 | agagacttgc tggacaggaa agctttggta aatcttcaga aatgatgttc aagctgaagg |
| 103321 | aagatgttca agctgcattt tggttgcaaa atgcagattt gcttgttttt ctgttgaaaa |
| 103381 | attcctctat gtgatgctgt aagagttacc aggctattat taaaagaaag atgtattgtc |
| 103441 | acactgagta tgtctgcaaa actttcttag taactggcct gtttgtttat actcaatgca |
| 103501 | catgttaaga tggctacata ggattcttac tatgttctat caatttggaa tgtaactcaa |
| 103561 | cattcttcca gcacataaag ttgtttacaa tatataatga gtgtgaaaga tataaataaa |
| 103621 | tacttctatt tgatgtctac accaacatta gatgaaaaaa tgaggtagtg agaagttgat |
| 103681 | atatatcctg taaattagca tatttaaaac caaaaatcct tctactgggt aaatattaac |
| 103741 | taggaaaaca ttctacagga tataatgcat ttttctagct ctgccccctg aaaaggtcta |
| 103801 | gtagtaatgt gatctgatgg cactaaacac ttctgatctc aaatgatagc ctctaaaatg |
| 103861 | ctgttttcca acaaagagaa ttagggctcc tgaagaaatg gctgatgcca ggtgtggagg |
| 103921 | agaaaatgag caagctgagc ttgtgatata tgttcacccc agagagggct catgtcacgt |
| 103981 | gggcaccaac agagaggggt ctcaggtagc aacagtgggg ccattggacc atcaaaggag |
| 104041 | caattagggt acttgactga agcctgtagg ttcataattt ttactctaaa acaaggaagg |
| 104101 | aaggtgggaa gaaaggggaa aggaggggaa gggaaggaag aaaggaggga gggagggaag |
| 104161 | gaaggaagta aagagctaga acaacaatga aacaaaatat ctttgataac tttttggggg |
| 104221 | agtacagagt ggaaattttg catttctgga ggctttacaa gatggggatc tgtgcttcag |
| 104281 | aaataagtca gtaaagatac atttacatag atgtttcctc cagtattgct tggaaaagcc |
| 104341 | aaaatcaaag tgatgtagat agttacctaa gagatgaata aaataaataa tggtacatct |
| 104401 | tttgtatgga atattacata gccactaaaa ataatggagc agaactctac tcaatgacat |
| 104461 | ataaaaatgt ccatgatata ttgtataatg gaaaaaaatt ttaaagcaaa aggttttacc |
| 104521 | ttatttattt cacaagctaa aaccctatat gattatacaa accaatgtgt ttgccttaaa |
| 104581 | aagataaaag tctggtgtcc tctaagacca taccacaaac agtgatcacc tctgggattg |
| 104641 | gaattttata gcagttgtgg gtagagagag tttgggagaa aacattcaca ttttatctca |
| 104701 | taattcttat atttttaaag cttttctatg agcattattt tgatacatat gtgagcagag |
| 104761 | atggtccctt ctaggtacca gttacaccat tgctaaacct cgtctgtttc ttccaaaggt |
| 104821 | gtcaggcagc gaacaagacc tgccccacca attacatgtg gaataatcac atctgcagat |
| 104881 | gcctggctca ggaagatttt atgttttcct cggatgctgg agatggtaag cagaatgatg |
| 104941 | ctttaaagac taaacgccag gggtctatat tgtgattaac atactctaat attaaaacca |
| 105001 | cacatataat attcatcatt cttctaaata agtgactaaa aaatagtaca ttttctactt |
| 105061 | cacgttggta ttattttgaa ctcctttata aaggaacata ctcatgtttg tacccaatcc |
| 105121 | tttgatggaa aacatacaaa aggaagatgc ttttaaaagc ttttgtttca tgccacaccc |
| 105181 | agaaccaagt aaggtatagt aaagattctc tgactatttg tctagactca acagatggat |
| 105241 | tccatgacat ctgtggacca aacaaggagc tggatgaaga gacctgtcag tgtgtctgca |
| 105301 | gagojgggct tojgcctgcc agctgtggac cccacaaaga actagacaga aactcatgcc |
| 105361 | agtgtgtctg taaaaacaaa ctcttcccca gccaatgtgg ggccaaccga gaatttgatg |
| 105421 | aaaacacatg ccagtgtgta tgtaaaagaa cctgccccag aaatcaaccc ctaaatcctg |
| 105481 | gaaaatgtgc ctgtgaatgt acagaaagtc cacagaaatg cttgttaaaa ggaaagaagt |
| 105541 | tccaccacca aacatgcagg taagagatcc tcatgaagaa tattaattta tcttactaga |
| 105601 | aatatgctct aaaccaaaag cagttttctt aaccaaagta acaattgctt gttagaatga |
| 105661 | ggccaataca ttctgaaaga caaaaatctt atattttttt aaccaatgct aaacttgcat |
| 105721 | gagactggat ttgcttccaa aaaagcaaga caggagcctt gaggatcaaa attagatcca |
| 105781 | agctcagaat atactctaaa ttagtggttc ctgtgggatg ttttcaaaat acctaaaggc |
| 105841 | aatccacttc acccccacca aaaagtagga tgccttcaga cataatttag gacaactgtt |
| 105901 | ctaaatgcag acaattttta tgcaaaactt acaaacaaag gacaggagag taccactggt |
| 105961 | atagaattat atctggaaat gaaaatacat agctaagtat ttgatagaat tagagaaata |
| 106021 | aatgaatatg ttgttaaagg tacccaagac tcatgtctta aacttggttt tgactcttct |
| 106081 | gcatcaagaa aaaaaaaatg aagttattcg actcaagcac attcagtgtt aatcccgaaa |
| 106141 | cctcttcctc tttaatctca gagaataaat tgatcctact atttaagcca gacctgtccg |
| 106201 | tttgacattt tctccacatt ccttcaacat tgcttcatgc cattccttct ttcttctgta |
| 106261 | acttgaacat cttcaactct actttttttc cccttagttt ataaaagttc ccaaatttct |
| 106321 | tctattttat tagcctctta ctgtgaagtt gagcaaactt tagcaaccca ggtctgttct |
| 106381 | cttcaaagct gctcaaaaac gaggcatttg ttttcacctc tttcacctac catgtattca |
| 106441 | accaactaaa acctggttta tggcctacag tgctatgtta aacctctgtt taacgtttca |
| 106501 | ttggcctcat agttgccaaa tccaacagag aattttgtcc ttaggtaacc tgtcctttcc |
| 106561 | atggcctttt tctttaagct cacctcttct atgacatgac attgtacttt cctagttgtc |
| 106621 | tacttaattt tcagaccatt tgtttttaat cactttcact ggattctctt tttacatcca |
| 106681 | ttctttacat atgggtagat aatgttcttt ttaacagaaa aaagcttgta tttcatttat |
| 106741 | ttactatctt cactaatgtt cattattatt tttccacagt atataacgag ttcatctttt |
| 106801 | tattctcaag agttttgtgt ccttatttta atgtatattt ttggtggttt tcctaaacta |
| 106861 | atattttagc tactggataa aaaggcttta gtggatctgg ctatctcatt gattatattt |
| 106921 | ttcttccctt atactgcaaa gtacatcata ggtataattt tattttgaga aagaactata |
| 106981 | ttccaatttt acctatatat tcctactgta ataaccaaga ggacatttca aaaatgtata |
| 107041 | tgctactatt ttcagaagga tatagatact aaatcttttt aagccttatt aaactatgtt |
| 107101 | ttatattgaa ttcttattga gtgaatttag tatacattga aaaatctgaa attcttatgt |
| 107161 | ctgcatacct acttttctac agaattatat gaacacagac aatatttcta tcattgcatt |
| 107221 | tctcctaata ctatacattc tttcaaaaag actctgttta ctgcaactca tcctagattc |
| 107281 | atttttctaa gtgtataatt ccaagctttg tgatataaat tggagcaaaa gagaaaatat |
| 107341 | catattttga aaatatatat tctcctcatt ctctcttaat atctattaca aagtgcagta |
| 107401 | ggcagagtat atattctttg gttcattgcc ttaattaaca gcttttatgg atcattaagt |
| 107461 | atcatatccc tttttagtag aggaatacca gtagtcataa ctatgaattt gcatttgcaa |
| 107521 | agtttaagaa aaattaacta gacatgcttt cttttctaga gccaaataaa tgaaatgtca |
| 107581 | aaaccaaatt gtgtagggtt ttttatagca cacttttgat ttccacctca ggtcatcttg |
| 107641 | cacttttccc tttacaccta ttcccacaca ttaggcatgc tcatactcca aaatgttttt |
| 107701 | aaaagatagc tccaattctc accaggcgtg gtggctcaca cctgtaatcc tagcactttg |
| 107761 | gaaggccaag gcgggcagat cacttgaggc caggagttca agaccagccc agccaacatg |
| 107821 | gcaaaaccct ggctctatga aaaatacaga aacttagcca ggcgtggcca cacatacctg |
| 107881 | taatcccagc tactojggag gctgagacac aagaatggct tgagcccagg aggcggaggt |
| 107941 | tgcagtaagc tgagattgcc ccactgcgct ccagcctggg cgacagaaca agactctgtc |
| 108001 | tcaaaaaatg aagcctcaat tcactggagc aacagggcag gagacacact gaccaattga |
| 108061 | taatttgtcc tctcaccgct actggaaact ggaagtctag gatgggagga aggggaccct |
| 108121 | cgctgctgag tattgtcctt ctcacctccc tgccccagaa tgcctctgtt tcacctggag |
| 108181 | ccaaggtgga aaggaagagt tagtcaccgt catgcagcca gctgccaagt agagacacaa |
| 108241 | gagtgagggt ggggctgctc actgaggacc acagttctgc caacatggct gggtttaatt |
| 108301 | gcctggaagt ctgggtccca tacaagtccc agactcatgt gaattctgcc atgcttatgt |
| 108361 | gatcacagta agtcagcaga ccttcagtgt tcactttcag aatttctgtt ttatgtttct |
| 108421 | ttcacccatg aagcaaagag cacttcaaag gaactaatgc ttacaatacc tcatccttcc |
| 108481 | atttgttata tcagcaagca agacttttat tttaaggctt ttccattcct cttaaatgtt |
| 108541 | gtaattctaa gcagaaacaa acttttttaa acctatgaac aatttcgtca taaaattagt |
| 108601 | aattttattc cagtctgaaa tttaaaaaca cagaaatacc ttggtagcat gatgaatcca |
| 108661 | ttgccttgat ctttaacgta agtgtgttct tgtttgttct cctagctgtt acagacggcc |
| 108721 | atgtacgaac cgccagaagg cttgtgagcc aggattttca tatagtgaag aagtgtgtcg |
| 108781 | ttgtgtccct tcatattgga aaagaccaca aatgagctaa gattgtactg ttttccagtt |
| 108841 | catcgatttt ctattatgga aaactgtgtt gccacagtag aactgtctgt gaacagagag |
| 108901 | acccttgtgg gtccatgcta acaaagacaa aagtotgtot ttcctgaacc atgtggataa |
| 108961 | ctttacagaa atggactgga gctcatctgc aaaaggcctc ttgtaaagac tggttttctg |
| 109021 | ccaatgacca aacagccaag attttcctct tgtgatttct ttaaaagaat gactatataa |
| 109081 | tttatttcca ctaaaaatat tgtttctgca ttcattttta tagcaacaac aattggtaaa |
| 109141 | actcactgtg atcaatattt ttatatcatg caaaatatgt ttaaaataaa atgaaaattg |
| 109201 | tattataagc tg |
| Human VEGF-D Gene (NCBI Accession No. NG_012509.1) | |
| (SEQ ID NO: 83) | |
| 1 | aagacacatg cttctgcaag cttccatgaa ggttgtgcaa aaaagtttca atccagagtt |
| 61 | gggttccagc tttctgtagc tgtaagcatt ggtggccaca ccacctcctt acaaagcaac |
| 121 | tagaacctgc ggcatacatt ggagagattt ttttaatttt ctggacatga agtaaattta |
| 181 | gagtgctttc taatttcagg tagaagacat gtccaccttc tgattatttt tggagaacat |
| 241 | tttgattttt ttcatctctc tctccccacc cctaagattg tgcaaaaaaa gcgtaccttg |
| 301 | cctaattgaa ataatttcat tggattttga tcagaactga ttatttggtt ttctgtgtga |
| 361 | agttttgagg tttcaaactt tccttctgga gaatgccttt tgaaacaatt ttctctagct |
| 421 | gcctgatgtc aactgcttag taatcagtgg atattgaaat attcaaaatg tacagagagt |
| 481 | gggtagtggt gaatgttttc atgatgttgt acgtccagct ggtgcagggc tccagtaatg |
| 541 | aacatggacc agtgaaggta ggtgagctca atttttaatg tgatgaagtt ctttttattg |
| 601 | gctcatacag aggactttct ttgccctcac tatttttgaa acaatataaa aatgcagaac |
| 661 | catcctagta gtggactgga caggatatat cctgcatttc ttggtgactg ttctgtttct |
| 721 | tagtcttatt aaacttctgg ccatgagtat ttgaaaatgc tggggtgggg gttggttgtt |
| 781 | ttttgaagtt atggtagaac atgagcagtc tggaatcttt ttggatggag aagtttctcc |
| 841 | toggttgcat atgcagatgg cacagtggtt ttttaatgaa acattgattt gtttttatga |
| 901 | ctgataactt tgtgggccat ttattttcag gtctttgtga gcagcagagg ttagtttatt |
| 961 | cgtttggtaa attgaaaatt gcagtgcttt ctatgaacat tttctatctg cttttgcaaa |
| 1021 | ttaccaattg ctgccatcca gaaaaatgtt ttctcagaga gtgtttggtt ttttcgcttc |
| 1081 | ttgaaaggtg taaaattcaa cactaatcaa atctttggtt ctgtgagcaa gcatcaaata |
| 1141 | gagcttaacg aacagagagt aatgcaaaaa attaaccaga atgttgctga ctgctggaac |
| 1201 | aatacttagc ccaattttat gcatttcatg gattatatat attttaagct tctattttga |
| 1261 | aaatgattcg gatgtaattg ttttgactga agtggtgcta aatcattatt gatttaggaa |
| 1321 | cttctggcct tgaaaagaca attaaatgtg agtttgagtt tttgtagctg ttgtttagga |
| 1381 | cgtgcatttt caacaggggt gatatcgccc ccagcagggg aaaattggtt cgtggaggga |
| 1441 | tgaaaaagtt ttaccctttt gatacatcac gcacagacgt acatacagta catatggtat |
| 1501 | gtagtttaaa atacgttata cattcaaatt gcataggggg aaagtgtctt caaaggctcc |
| 1561 | tggcbggtgt tggtggcctg gcgagggtgg gggctgatat ttaaaaatgt tgaacactga |
| 1621 | gctggaggtg agtgggaagt caccttgtct ggcagccagc acttttctaa cttctcaaat |
| 1681 | tccaaattta catgggagaa atcacagtgt ttggctccat tttactgcca ccagccttga |
| 1741 | tattggatct caaggccaga ttgtaggtaa atgggaatct gactgtaagc ataggtaaca |
| 1801 | ggagaaaacg gtctgacttg tttgaagcaa gtaagacttt tccgactgtc cagctttttc |
| 1861 | gtaagttcat gaaacagaaa tcctttagat gtgctgggct cctcgagttt taaaatattt |
| 1921 | ccattttcct tgtaaaacat cactttatct gatggtataa taaaggagac tgaattgttg |
| 1981 | gacataagtc atattcttta ggttgctcag ctctttcaaa gagcacaagc accaagaatg |
| 2041 | tctcaggtgt cctgggcact gtgaagtttt atttatttat ttatttattt atttgagacg |
| 2101 | gagtctcact ctgtcaccca ggctggagtg tagtgatgca atctcagctc gctgcaacct |
| 2161 | ctgcctccog ggttcaagtg attcccctgc ctcagcctct cgagtagctg gaattatagg |
| 2221 | cacgtgccac catgccgggc taattttttg tatttttagt agagacaggg tttcactgtg |
| 2281 | ttagccagga tggtctcgat ctcctgacct gatgatctgc ccgccttggc ctcccatagt |
| 2341 | gcbjggatta caggcgtgag ccaccgcgcc cggctggcac tgtgaagttt taaatgcttt |
| 2401 | gaatgtactg ttaatatata aatttgttga atggagtaac gaaggaaagc agctgaattt |
| 2461 | gcatgtgaca gaaaacaagt tgttacacgt ggaggatgca gcattatata cttactaata |
| 2521 | cgattagcaa ttttttttca gaatatatca gaatttcctt taaaaagtga agctacatat |
| 2581 | actgaactta tatccaccct acccctcgct caattaaatt atttctgata tgaaggaaag |
| 2641 | gattgcctat agctaaacag aagttttggc atagatatga ctgagtggta agggataatg |
| 2701 | agatatataa tattggttat agttttcttt tttccccaac ttttaagaaa agaaaatcta |
| 2761 | agaggaaata atgtttctaa atgttaaagt tcttcttgtt aaaattaaaa tgtagtgtga |
| 2821 | aaacatattc atggtggtaa tggtttaggg tggtggtatt tgaatttttt gttttttttt |
| 2881 | ttttactttt tgaaataaaa tgttttattt attataagaa attcatggtc ttcctcggta |
| 2941 | cttggtccat atgtatttct ttctcctcac cctcccctct tctaaccacc tcttttccag |
| 3001 | aatggatggt gatgccaatt ttgctcctag gttgtcctcc agccaccccc agcccagttt |
| 3061 | tcactttcta gctttgaaga ttgtccagtt ttccagctta gactagcatt gcctctacga |
| 3121 | tagttctagg tatctgtgaa gatttttttt tttcctgtag caatcatgaa atagctacta |
| 3181 | gtgctctgga cgattgtgct taatgccatt tgggtgtaag gcctcctaat ttcattagtc |
| 3241 | ttaccacttg bitttgcaag gatagctgtt tggagtcagc agtgcatgga acatgtgaac |
| 3301 | tgagacaaat ctgtttgaaa atctcaactt aagatttcaa atccactgtg tatactattt |
| 3361 | agcbgttaag tttctcagtc taagaagaac acgttttcct ctagaaataa gcttaaccca |
| 3421 | gtacctatgc atcgttcatt aatataatca gattacggcc tagatgtgga atttatttct |
| 3481 | gtttctattt tctccagctt tttaatacag ctcagacagt catatacagt tgtacacgat |
| 3541 | gtgcactgca caactctagg gtgctccatg catatagaca tatctgtatg tgacagccct |
| 3601 | aagtacaatg tggtagacac tctggacatt tggtaaataa gtatctacat cctcactctt |
| 3661 | ctctcattca cattggcctt ctggccagca ctatttctgg tttatttaat aaacaattct |
| 3721 | tgtcccccag aaattctgtg agtcttctga agatgccggg gaaagccagc cttcataaat |
| 3781 | cttttcaatg tcactaatga agacatgtca ctattgcaac aaaggaatac caggccatag |
| 3841 | taggctttca caactttttt ctgacagaat ttgtttcttt ctgctttatc ttccctgtgt |
| 3901 | actgtctttt tttatccacc ctctttctta attacactgg tagaagaata ttggtgggtg |
| 3961 | atggagtcat tcacattctg tgtaagacta gaatcagtcc aagatccaca ggaatgtggg |
| 4021 | atacttcttt catgagaagc cacatggctc acaagacttc tttacatttg tgttacttgc |
| 4081 | cttctcctga aattgtotct acagtgaatc aaataacaag gactgaggtg tattttccat |
| 4141 | atttaattcc aggaagcagt cccactcctt ctccctgccc gatttccagt tccaaagtca |
| 4201 | tgtacaggga agtccattga ctttgatgtt ggtcaaatgg cccagcaagc ttttgtccac |
| 4261 | ttttctgtca gacacagtcc ttggcacaaa agacatctga aatacttccc attaatttct |
| 4321 | tctttattta tgaccttgta gttaagtgtg ttgaattata tgcttctata cacatctgtt |
| 4381 | ttaatccagt tactcactta taactggtga ctgaaacttt tatatcacta tagcgtatta |
| 4441 | ttggtaccca ggatacaata gaatcctcaa tatcttacat cttcaatata gtacttcctt |
| 4501 | gtatgactta tttcttaaca taaaaatcct gttttagacc tgtggataaa cctttactat |
| 4561 | aaatcccaga gacaatttct ggaatgagaa tatagtattt gtccacacac atgattccat |
| 4621 | gtagaacttg aggtaataag atctgggact cccagataca tttattatta ccacaattgg |
| 4681 | atagaggtgc tacagaaata ggcacacaaa tgacagtaaa attgtttctg agaacatttc |
| 4741 | taggctcaac ttttttctac aacttaggat gaacagagag agtggaaact gtatataatt |
| 4801 | gggaatgtga ctgagatgag acaaagatga catggaagca tttcttcttc ccttgctttt |
| 4861 | ctggtggtaa gttcactctt ctgtcttcct tgccactagt tcttgcagtc cctaccaaga |
| 4921 | cactagggca ttatacagca aacacaccct gaggccccct gctactgcat catgaaagcc |
| 4981 | ttagctggtt gtgatgtcct agttcataaa gaatctgcct agtctctcat tagagctttc |
| 5041 | caatatttga caattcattc atttattttt ttcaacaggt atttattggg caccaatata |
| 5101 | tctaggcatt gatctagggg ctgggaatga agcaggaaat aagtctaggg gctgggaatg |
| 5161 | cagcaggaaa taagacaatg tttcaattct cttggaattt gcattccaag tgtgatgggt |
| 5221 | agaggcagac aatatgcaaa tgtgtaacat gtcaggtgtg ataaatgcta tgaagaaaat |
| 5281 | caagtagggg gagcggatac agtactgaat aaatcagagg agacctttct gattcagtta |
| 5341 | tgtctctaca gagtcctgaa ggtaataaga gagacagagg caaccatcag tgcagtggcc |
| 5401 | atggtgggcg cagcaagttg gcttcgatgt ctgggtatta gccagggagc cggtgcagct |
| 5461 | ggaggtgggg tgagtggagg ccagaagtgc agttgttgag gttggagaag tagccagaga |
| 5521 | agatcatgta aggtgtgact gtgctcccat aaaactttat ggacactaaa aattgaattt |
| 5581 | tatgtaattt tcctgcatca tgaaattaaa aaaaaaattg tttcaaccat tggaaaatgt |
| 5641 | aaaaaattgt tcttagctaa caggccatac aaaaacaggc tgcaggttag atttggccca |
| 5701 | tgggtcatag tttggcaatt gtgttatata taaattatta cactgtttta tcttcatcat |
| 5761 | ggtacacatc catacctgac atgttttatt tgcctgtgta tacatttatt gctttgtcac |
| 5821 | cgtctactag aatacaagtt ttttgagaaa aggagctctt aattgtcttg tttactgcag |
| 5881 | catcccaatc accagtgaaa agcctgtatg taatagagta gatactaaaa acagtatttg |
| 5941 | ttgactgatt gatggactag acctcaggat ttttagatac ccaggatcag gatatgaact |
| 6001 | actgcattaa atcatctcca acatctactc tcatgatggc ttgtctgact tcatgattta |
| 6061 | aaaaaaattc taaagcattt ttaaggcagt atattcaccc tgtgctattt tcctgtgggc |
| 6121 | ataggatagg gtttgtgtgg atgtatcaaa gacttctcca ctgtccttcc aggcacaagc |
| 6181 | catcctataa ggcgttccct tttaggagcc agaggtatag aaggaagtca tctgagccca |
| 6241 | ttattccagc ctgttggtaa cagaaagaga gatgagcaaa gtagcatttg gctgcacagg |
| 6301 | ggtctggaca aaacttcaac atcttgatgt tttccttcat ccctgagagc cttagaaatg |
| 6361 | ctacctatgc tttcatttta bittaggttg tttgtaaaat catggtaaat tatggcaaaa |
| 6421 | tactctggtc agatacttta tcaggttagg ccaggatatg ctgtgaaaac aaacaccccc |
| 6481 | gaaatcttgg tggttcataa acaacagaga cttatttatt gcttatacca catgaccagc |
| 6541 | ctggttcatc tggggtcaaa tccacatggg gatgaagctg gctgagtatc cgatcttgaa |
| 6601 | tgttaggaat cactatgaca gagggaggga gagcctgaga gcgcctcacc ctggcactta |
| 6661 | aattctctgg cccagaagtg acacatatta cttctagttg cagccccttg acccactgcc |
| 6721 | ccatctagcc acaaagggcc aggaagtgca atcccaccat gcgctcagaa ggcagagaga |
| 6781 | tggaaatatt tgacaaaccg tattaatgac tgtgacacat ggtgagttct tcacattggt |
| 6841 | gacttctaag tgcttctgaa cacacctttg actgtagaat gtcaaaaaca ttctcaattt |
| 6901 | tacagcagtg ctggaaaatg aattgtattg agacaacttg ggaggagtag aaggagatgg |
| 6961 | aataaaaaaa attcactgat ttctgtcatg tgtcatgcct cctttaatgc acggaagaac |
| 7021 | gtatactttg cttaggaaat cacattacca cattcagaac aactttagtg aagatgggaa |
| 7081 | acaaccatgg ctggtcaatg gggtaacgtg tgctgaagga agacaggttt atttctcagg |
| 7141 | aggcatcttc tccctgggaa aaggaaagac acatattcat ctcagtaaat atttgtggaa |
| 7201 | tgtctaccat gtgccaggca tatgagtgga aatgatgttt ttataaaata aagatgtctt |
| 7261 | aagtatttgc atgtcttact aacagtcagg gagttcaact tggccagtgg gaaggataat |
| 7321 | tttatgcctg aaactttagt aatgtaaatt tttatgctga ttctcttttt gtcattcact |
| 7381 | gagtattcct aagtaactat aacactcaca tattggagat aagtattggt ttcctgaaat |
| 7441 | ctaggtttgg ctaacattgc ttctggaaag agaaaataac abitgttgaa tgtccctatg |
| 7501 | gcaaattctt gctgttactt actgttccat tatggagagt gttagaaaga gtgtaatttt |
| 7561 | gattgaatga atatgctaaa aaaaacaccc taaataagaa actcacaaaa agaacagagt |
| 7621 | ggaatgaaaa agtgaaaaca actttgcaat ggtgtgcttc gtttataagt acattgtcac |
| 7681 | agcaagacta gggatgattg ggcatcttag agagtgcaga ggatgttgat aaggaagagg |
| 7741 | aagaggagat aagtggattt tgatatggga cactagatcc ttagggaaga aatgtgaaag |
| 7801 | agggtattct gggctgactg ccgctggcat agcaaggctg ctttgtagaa gggacaataa |
| 7861 | agttttgcat aaggatcctt tttcccatcc aatggattat gctatacttt agctggaata |
| 7921 | gggctgtagc agttagctat tgccacaata atgctgtgta acaaagcagt ccaaatttca |
| 7981 | gtggcttaaa acatcactac cttattctca cttatgtctc caagtcagct gggagtcaga |
| 8041 | ggattaaggc tcaaggctta ggtttggtgc aggtcagccc caggtgtctc tcatcctcct |
| 8101 | ttgatcagtg ggctagcagg agcatgtgct tgcaatgatg gtggaaatag aaaaacgtgc |
| 8161 | aatcatatgt gagtttcaag cctctccttg catcatgtct tgtaatgtcc tattggtcag |
| 8221 | tagggcbjgg aaatatatcc ctccctcaga gagtcaggag tggaaatgaa aatgttttta |
| 8281 | acaatagcct aatcaccata aaggctttgg gtggcacact gggcccttga tcttggagat |
| 8341 | cagtgataga ctgtaacaga cctcagaaac cccttggggt tattcagagc atacttctag |
| 8401 | agggtctggg gttgtcttgc cattcaagcc catcccacac ccatctgccc ccacatatcc |
| 8461 | taagcacagt tatgtcaagt gagaacagag gcatttgagt ggagcacaga tttcaaacac |
| 8521 | ataaaggcag ggcatggcca tctcccgcag tgctagatga tctggcbggg agaaaaccac |
| 8581 | aagcagtctt catcaaattg gaggatgtgg aggtcccagg catttagtcc cctagtatgt |
| 8641 | ccttaaaaaa cccagacagc catgcacaca cactcacaca aatgcacacg cacacacaca |
| 8701 | tacattcaca cacacactct tttcctgcat ttgtagttcc tttcctcagt ctaagtttaa |
| 8761 | ttaaacaact tggatttcat atatattcat ttaaatatag aattgcataa agaaaagagc |
| 8821 | caccagaacc accaatggta gcatgaggcc tctgaaatga aagatgagcc caccccaacc |
| 8881 | ttaaatggcc aggtttctgg atttcagaga agcaagatca gacccttggc aggttccatc |
| 8941 | tgagcagcca cagcagccat agctacagca ataattatta gcaagtgtgc gttctccctg |
| 9001 | ccctagttag tacaccctat accatcaggt gtcatctgtt ctagttaaag tcagaccgta |
| 9061 | ataacaaaac atcaaaatgt aaaatgtggg ttgttttttt ctttacacac ttctaaagac |
| 9121 | atttaaagcc tgagcagata ccttaaaatt catcttctat ctgaaaatac ctcctagaac |
| 9181 | cattttaaaa aggagatgga ggcaaggtgg agagaaaaag tttgtctttg gttgacactg |
| 9241 | ggaaagttgt cttttaaaaa attttttaat gtttatttac tatggagttg ccgtgtcttg |
| 9301 | ctcttttcaa gctcacctgt cojgcttcta aggaatatca ggggccctaa aacacagact |
| 9361 | tctaaacccc aacacatagg acacacctaa gtgtggaaag aaaggaaaga ggcaggaaag |
| 9421 | aaaggaaaga ggaaggaaag aaaggaatgc aacatttatt gtgtgcctac catatacctg |
| 9481 | accctttcct acatatgacc acatatgatc tcttcatgag ctttatgagg tgacctatta |
| 9541 | ttaacaatgc tgctttataa atgaggaaac caagccttaa ggaagtcaaa taccatatcc |
| 9601 | tcagtcaaat agctaaaaaa aaaaaaagaa aagaaaaaga aaaggtagtg cctggattca |
| 9661 | aagacagctt atttttttaa cttatagagt aaaatggatt ttttcccttt tggtgtgtag |
| 9721 | ttctatgaat tctaacacat gtatagattt atataaccac aaccataatt aggatacgga |
| 9781 | actattccat caccccccaa aaattcactt acgttatcct tttacagtca gaccttcctc |
| 9841 | ccagcctccc aacctctggc aatcactgat ctgttctcct tcactgtcgt tttgtatttt |
| 9901 | tggttcgctg tcattttggt tttttgggaa tgtcatataa atggaatcat acagtatgta |
| 9961 | gcagtttgag attggcttct ttcactcata ctgcctttga gattctacca gtgtcccagt |
| 10021 | agttcattcc tttttattgc tgagtagtat tacatcgatt gctgaaccgt tcacccattg |
| 10081 | aatgacacat aagttgtttc tagttttttg gctattgtga ataaagctac tacaaacatt |
| 10141 | catgtacatg tatttatgtg aacataaggc ttcatttatc aagggtaaaa tccccagaag |
| 10201 | taggactgtt gggtcatatg gtgagcatat gtttataaga aactgcaaac acattttcca |
| 10261 | gagtgttata ccattcaaag ccagttttga acaattcaaa tctatgcttt ttctttccag |
| 10321 | taagcbgttt ctccagacga cagagtggtt tactatatta ggcaaagctt catcttgggg |
| 10381 | aatgttattt ccaactgaga gctatgggag gcaaagtcac aaaccttcag ctcagactca |
| 10441 | ccaatctaac ttgatctggg aagatacctc gacaggtggg ggagggagaa catctgctga |
| 10501 | ggaaggccgg gagtggagtg agacattgct acttggaccc caaaaccagg aaaaatttgg |
| 10561 | tggttttggt tgagcttgaa ccatcaggtt aaacaggaaa tgcaaaatgc tcttccagtg |
| 10621 | aaacagaaga agctgctaag aggtagaggg aacatggaac tatttgtatt gcaaccgaat |
| 10681 | ccacctcccc aaacccactg caaagggaaa ttgctgactg caatgtccct gaactccagg |
| 10741 | gtgagtaaga agagaaccac gccccccagg gaacacttgt aaatttttaa gcaaatctag |
| 10801 | ggaaagttgt gtaaaatgtg tcccttccat aactcaccct aaagcttctg ggtattctaa |
| 10861 | atatacaaat tctattcatc acaccgtttt atcaccaagc ttcctgaagg catattgcca |
| 10921 | acacaatttg acaaagaaaa tagcaccaac agotttttaa agccttggaa ggatagtctc |
| 10981 | agacccatgg ctctctagag gtccagggat gaatctagca aagctggcct ccttttccat |
| 11041 | atatatagaa caagaggatt gttaatagca tgggattgga atggctcaag aaaagctttt |
| 11101 | tgaagctctt tgagaaaagt agccagggtc cccccttaaa ccttctttgc tgatggcctt |
| 11161 | acagctcaaa accagcattg gaaccggcta ggtactccca aaccaaagcc tcttccactt |
| 11221 | ggcatgtata tttatattct catatcttct ttcaaatacc tttcagtgtt gccttttgaa |
| 11281 | aagattataa tttgtacttt tttgttgaat tcagtaggtt aaagcagtac ctattatctt |
| 11341 | taattttatt ttgaaaaatt ggttggtttg gcttttgggg aatttgagag gcaatttagt |
| 11401 | ctgtcattta ggatggcatc atataaggca gccaacttca tcatgctatt tagcctgaaa |
| 11461 | agcgatattg ttgatcttca accttgaaaa agtcgtacag gttgggagct gacctgcaaa |
| 11521 | tggtcaggta tcttgagata ggaaaaggat ttacaaatcc aggtcagcaa gatactcata |
| 11581 | caaatatttt gaacccaagg tatacatcaa atagatttaa agactaagac tatttctgag |
| 11641 | catgcatcga ctgtttgaag actagtggac ttgtatgaag ttcaggagtt aacctgggtc |
| 11701 | ttgtaactta aaaacagcat ttctcatgcc ctaaattagt agtagagaga caatatagag |
| 11761 | agttcctata tagccctcac ctatcttcct ctaaggctaa cacctgggaa gttttttttt |
| 11821 | aaaaaatcct gatggcaaag ctgcagccca gacccaaaaa atgagaatct gtgagattta |
| 11881 | agattcgagc atcagtattt ctttgaagct tattcccagg tagattcaag aagaagccaa |
| 11941 | gattttgaat gcctgcttta ttattttttt ttttgtgaga gctttaatgg cacaaaatgt |
| 12001 | ttatagctac aagttataca tgtattgtaa attgtatata acgatacaaa gtgctaacta |
| 12061 | aatagatgga gaacgcataa ccactttggc tcagttaatc caataatttc cacaatgggt |
| 12121 | tcatgaaaaa cctcatctga ctggctaagt cttcaattct cattttttta gaatagtaaa |
| 12181 | tatttttact gcaatcacag gaacttgttg cgctttccaa tgtgcaagtt tctgcagttt |
| 12241 | tgctcttgat tctatttaca aatatcttaa gccagttttc atcaaacata tgaaaaatga |
| 12301 | agcctgcctt tttgtcccca aattgtaaat aggtgtaaat ctttttaagc atcaatctca |
| 12361 | caatttcaac agttaattgt tgaattctcc tttccaatac acgatgtttt cgttcaacac |
| 12421 | aaatctgggt agtagtaaaa tgttcatccc gtgtgttagg gctacagata catactacgt |
| 12481 | gctctcataa taaattttac tgttatgatg tattcatttt ttaagtaatt ttccataatt |
| 12541 | tcactgatgc tttaagaaaa agagcaacac tatgtttctt tttaacgttg aaagaaatca |
| 12601 | taaatatacc caggttcaat ggcgctactg atagaactgc tgctgaattt ggaatgagca |
| 12661 | caatgcacta gctgtaagaa gtcagttttg ggtaatgaat aattttactt ttccctcctg |
| 12721 | taataccaat ttcatactgt agcagtttat ctagtcagtt ctcctaactt tctgtccttt |
| 12781 | ccaattttat tcaggctttt caaaaatcag tgagccatca gatttacttt cttcccaaag |
| 12841 | aaattcaaat atgaatgctt gaaataaata ggttatagaa ggcaaatcaa gtgttatcag |
| 12901 | gtttaagatt aagacagaaa catggtctaa taaaaagatg tccaaaatta tctagacatc |
| 12961 | aaatgctaga aagaaaaaag aaaattactt gcaaaactat ttaggatata tcatctttct |
| 13021 | taaagattgc cagctatatt cttgaaaaaa agataaaaat tttcttgttt aacctgcatg |
| 13081 | ttattttcca ctataacatt aattttacaa atgttgatgt gagagagaag agttagatag |
| 13141 | tttctgcaaa ctggcaaacc tggctgtgat agtgttttta ttcaagttaa cctggtgcac |
| 13201 | tttatagatt gctagottta aaattaacag tgtgaacccc tgotttaaac ccttctcaaa |
| 13261 | gtgcagtcag ccaatcaaca gtattgccat cacctgggaa cttgttagac cccactttgt |
| 13321 | caaactcatt tcctggtatg acaatgaatc tggctacagc aacagggtgg tggacctcat |
| 13381 | ggtccacatg gcctccaagg agtaacaccc caggaccatc agccccagtg agagcacgag |
| 13441 | agaaagagag aggccctcag ctgcttggga atccctgcca cgctcagtcc ccaccacact |
| 13501 | gaggatctcc tctcctcaca atttccatgc agaccccctg aagagggaag ggcctaggga |
| 13561 | gccccacttt gtcatgtacc accaataaag ttccctgtac tcagccaaaa aaaaaagaaa |
| 13621 | gaaagaaaaa agaaaaagaa atgcagcatc ctgtcttgcc ccaaacccac tgcgtcagaa |
| 13681 | tctgcatttt aacaagatcc ccaggtatgc acattaagtt ttgagaggca gtactacata |
| 13741 | taaaatatgc aacagaccac acaaacctgg gcccaattgc tatttgtcag atacatgtat |
| 13801 | gtatttatta cttataaatt ccttcatcag atatgtatta cgtgcttatt actttaaaaa |
| 13861 | gatgaatctg acacatatcc taccctcaaa gagatctcag tctaggaagg gaaatacaat |
| 13921 | gtgttcataa ataatatagt ctaagttagg gacttcataa aatgcttgat ggaagaagtg |
| 13981 | gcatttattt tggacctgga atgagaactg aaagaagata ttcaaaagac aagtgatgta |
| 14041 | gtgatgtagt tttgaataca cgaataatct cagggttact ttttctatag cttttggtga |
| 14101 | atcaagttgt ttattcatac atttaataaa tatttattca gctcttacta ttgctataag |
| 14161 | acagatacca ttactgctca ggtggttttt acaacctagt ggggaagaca agcagataac |
| 14221 | aagaaatgac acaattcatt agtgttactg gtagcgttgt gaaggacaag gatggggtgc |
| 14281 | tagaaatcca tgtgacggac cagacctagg caggggtggg agttatggag gaggtttccc |
| 14341 | tgagtaggta acatctaagc tgagaaagat ttagttagac aaagagtgaa tggcattcca |
| 14401 | ggcaaaggaa acagcatgga ctaaggtcaa aacaagaaga aatgaacgaa agagcaagtt |
| 14461 | gataaaatgg atgtccaata atattccatt gttcaactat gcaacatttt tcattgcctc |
| 14521 | ccaaattgga tcaaagtagc ctggtcttca tgcctccagt ccttcctctt tttaaaaatt |
| 14581 | ttatacaact atattaatat agaatttaca tactataaaa tttcattcaa gtatattagg |
| 14641 | aaatttactg agttgtatag ctatcatcca tataaggtcc gttatgtccc tttgcaatga |
| 14701 | accctccatt ctcactccca gcctcaggca gccaataatc tattttctgt ctctaggggc |
| 14761 | tggaaactta ggattggaat tagttggttg tataataaat ttagcttatt agagaactgc |
| 14821 | tgaactcttt tccaaagcaa caatacaatt ttacagtttc atcagccttg tgtgagggtt |
| 14881 | cctgttgctc catatccatt acaacacttg gtattgtcag tccttttgtt tatagccatt |
| 14941 | ctactgggtg tgaagtggta tctcatttca ccagcccctc ttcctaactt atttgccaga |
| 15001 | ttccccactt ctaaacttgc tctatgccca accaaagtga accccttgct gttttttttt |
| 15061 | tcccctgagg ctcgttgata tccccaaatt atgcctttct tcattctttg aatgtgttac |
| 15121 | ttgactggat atgtattttt ggtgaaggag aggggctgga aattgagaga tgaaattggt |
| 15181 | aggagtacag aaggccgttg acttcctcac aacgtgggac acttccaagt ttctcctaca |
| 15241 | aaccccacca ctataccttt ccaaatatct tgcctgccat cttggtcacc cgtgacctaa |
| 15301 | attaatactt aggaagctat ggagatgctc atgcttcctc aggggagaat gctgtgtgaa |
| 15361 | cttagaaatt agcaaaaggg gaaatgacta cccttgaaat tgtaccacag aaaggagcat |
| 15421 | acattcaaat gatggtgaag tcttattaaa aagactttaa gcctcccaat ttagaagctg |
| 15481 | gottgtggta gccaagaaat tagagaatga tttgttattt aagagtgaaa ttgctagtag |
| 15541 | tcttgaatta attaccataa aaaagctgcc gctgtaataa aaagcagaga tctggtaacc |
| 15601 | ctgcttcatt cactttcttc ctcttgatga aaacctttta attattttga ttttgattag |
| 15661 | cacttccatc agaaaatagg aagaaattag gagcaattta cttggcaaag gtttagtaat |
| 15721 | taaaaccact gaaatcaatg gtttaagtat gtatttggga taaacacatt tttaattgtt |
| 15781 | atttttgtct tacccaagtg gataacataa agttcattat acaaaaatca accagtgcag |
| 15841 | aaagccgatt gggaacaaac aaccattctt gaaccctttg aaaatgtatg cccttgatga |
| 15901 | attacttgtc aggtttctac ttaagtttaa ttgttagtac ttccacttaa aatagcaaaa |
| 15961 | tgactgtctg caatttctgt tatttcaaac taggttttaa attctggggt tgaaagtaag |
| 16021 | cttaaataac tctgagtact tgagtaaaca ctgcattttt tatgatgagg caagttgtat |
| 16081 | gagccattta attactttaa ttcaatccag agatttgata ttttctcctg agttctcaca |
| 16141 | gttggatagt ttaaaaatat ttctaagatt gaattgaatc ctgtaagaac tataataaaa |
| 16201 | ccatacatct gcctgttttc tttctttctc tttctctcct ttcttttctt tctttctttc |
| 16261 | tctttctctc ctttctttct ttctctttct ctcctttctt tctttccttc ttttctttct |
| 16321 | ttctttcttt tctttctttc tttctttctt tcctttcttt cttctttctt tctttcttct |
| 16381 | ttcctttctt tctttctttc tttctttctt tctttctttc tttctttttc tttttctttc |
| 16441 | tttctttctt tctttctttt tcaagatcgt gctccattgc ccaggctgga gtgcaagtgg |
| 16501 | cgagatcatg gctcactgca accttgacct ccccaggctc aggtgattct cccacctcag |
| 16561 | tctcctgagt agcbjggagt acaggcatgc accagcatgt ctggctaatt tttgtatttt |
| 16621 | tagtagagac agggtctcac catgtttcca gtctgatctt gaactcttgg gctcaggcaa |
| 16681 | tcctcccacc tojgcttccc aaagtgctgg gattacaagt gtaagccacc atgcctggcc |
| 16741 | ttgtattttt ggataatatt gagtgaaatt tgcctcaata cataaaaatc ctttcaatat |
| 16801 | gcttcaactc taaaatcttt aaaaagctgc ttggtttcat tattttcacg tggaagcagc |
| 16861 | attgtcacaa agttttattt tgaagactga attgtatatg gagtactgtc caactagaag |
| 16921 | agaatgaata atcaggaaaa gagagactat tgacaaataa gacttaatgg agaagcattg |
| 16981 | aatttcaaag ctgccagaga cattagcaat tgttcagttt catctccaga tttcttagat |
| 17041 | gaagaagctg tggcaagcca atttcacaaa cccgatctat gatcctcatg ctccccactc |
| 17101 | ctctcactcc cacaattttg ccttgattca aactttatcg aaaagtgaga aatcacaggt |
| 17161 | atgagctcac tgagcttccc aggcagtctt gccatctaca aacccatgtc attctgtatt |
| 17221 | ttcctcttgt ttctgacagt gatcctctca tctagtcaca ctacatccac ttatctctgg |
| 17281 | cacttatgtc aaaacaaccc ctctaggtcc tgccttagct ctcctgcata tgctttttct |
| 17341 | ttcttcctgt ctgttgcctc tttcccccag cttataattc tcccgctttt attaaaaaca |
| 17401 | tgcacccaca gacacacact cacatctgga ttcccaagtg cgataacctt gtagattcat |
| 17461 | attttcagat cgttcttagt ccacagaata taaaattgat tgaatgatgg caaacatctg |
| 17521 | cagatgacaa tcatttacag aaatggcact aaagccatga agctgaagct gcaggactta |
| 17581 | tggatctggg ctagaagaca ggggccatca aaaatttagc ttcagttgga aatcggctct |
| 17641 | tttagtgcac tacatcagat ccatgaaaga ggaaggaaga gttactgctc attgctgcct |
| 17701 | ggagctgtga cttctatgaa attacttaac ggggcaaagg aggaagcaga tacttccttg |
| 17761 | caactaggac gtgagctgag ctgatocctg aatcataaat actcaacaag attcggattc |
| 17821 | aggacccagg gtattatggg gtacaaccat aaaacttcta aatgttggag aaagaataat |
| 17881 | gagaaagaaa aatgaatctc tgaatgaaaa tggaactact aaccaaaatt ccaaatcacg |
| 17941 | ttaatataat tattactaag aatgacagca acaaaatgaa taatcagtat atgtcatacc |
| 18001 | tataaagtta aaatctaaaa taatttgaag aaatacttaa aaatatttgc aaccctcaaa |
| 18061 | gagataaaga aataatattc ataaaattga aatgacaaac taggggcagg gcgcagtggc |
| 18121 | tcacgcctgt aatcccagca ctgtgggagg ccgaggtggg tggatcacaa agtcaggaga |
| 18181 | tcgagaccat cctggctaac acggtgaaac cctgtctcta ataaaaatac aaaaacaaaa |
| 18241 | ttagccgggc gtggtggtgg gcgcctgtag tcccagctac tcaggaggct gaggagggag |
| 18301 | aatggcgtga acccgggagg tggagcttgc agtgagccga gatcgcgcca ctgcactcta |
| 18361 | gcctgggcaa cagaatgaga ctgggtctca aaacaaacaa acaaacaaac aaacaaacta |
| 18421 | ggaagaatat acagaaatgt aacagaaagt agataggaag aagaatgaag tagaaatgct |
| 18481 | tgaaattaga aatacggtga ttgaaattaa aaccttaaca aagtaaactc tagactcaca |
| 18541 | acaattggag agttggtgat tggaaagatg aaccaattaa tttacccata acacattaga |
| 18601 | gagaggtgaa gaggaaaaaa tagctaaata taaaagcaca tttaagagac atgtaagatg |
| 18661 | attaaaagcc tccaacattt ttcaaaaaag agagtagagg caatattata ggtggaataa |
| 18721 | tgaaaatgat aatggctgag aatattaaag aattgagaat aaaaaagaca agtggcctca |
| 18781 | gattgaaact gccatgaagg tctctgtctc aaattctcaa ctctgctttt gtagtatgaa |
| 18841 | agcagccgta gacaataagt aaatgaatgg acatggctgt gtgtcaatac aacttgattt |
| 18901 | acaaaaacag gcaggcagca ggctaaatta ggcbgggagg tcatagtttg acagctcttg |
| 18961 | atctaaagaa taaccattgt tggccaggcg cagcggctca tgtctgtaat cacaacactt |
| 19021 | tgggcggcca aggcgagcag atcacttgag cccaggagtt caagaccagc ctgcgcaaca |
| 19081 | tggcgaaacc ccatctctac taaaagtaca aaaattagcc gggcatggtg gcaggctcct |
| 19141 | gtagtcccag ctacttagga ggctgaggca ggagaatcgc ttgaacctgg gaggctgagg |
| 19201 | ttgcagtgag cggagatggt gccactgcac tccagcctgg atgacagagc cagactcggt |
| 19261 | ctcgaaaata aataaagaat aacctttgtt tacattttca tgtatatgtc tccagatgtt |
| 19321 | cctcatatat ccatataagt atgtatatat gatgtacata agtgcataag taaaaataca |
| 19381 | taagtaattt taaaagaggt tatatcatag ctgaatcaca catgacttag accacacttg |
| 19441 | ctcttctgag tacccatttt tggacaactc tcagtgtcca ctagcatgtc taaaaaaaat |
| 19501 | ttctcctcct aaaatgctaa tgcatgatgt agattttaac tacagctttt gaggctcagt |
| 19561 | ggaggagagt gtacatcaca ctacccaatg caaagccttt taacagcccc ttttgctcat |
| 19621 | ctttgttttc cttcactgtt cctcgccctg cagcaggcag ctgacattca cctttccctt |
| 19681 | ccaccctcct ctctcagcac tggtcctttg cctcctccct catcccagct ctacaaatgg |
| 19741 | cctcagcctg catattccaa ccacccagca aaacaaacat ctagtaacta ccgggtcagc |
| 19801 | aagagaaaat cggaggccct agccatagat agatcacctt atcatttctc cagcacattc |
| 19861 | caggaaggat ctcaattcgt tctgtcccaa tatacctcta catcctttgt tttgatgatc |
| 19921 | acatagaatt cattgtatgc atatatcatt atttatataa ctaagccccc aattttgaac |
| 19981 | aagaggctac atactgaaaa tcttttatct accaagtctt ttttttctta caaacccatc |
| 20041 | caatacagtt gcactgtatc cgccaataca gttcatgcaa ctgtatggaa agcaaactct |
| 20101 | agaagagtat ttgatagctc cataagaata ttaaactctt atacttctag agcgtaaaac |
| 20161 | cccagaaaag tattttgtca gaaaaaaatc atgtttttga aatagtaggt tttaaaagaa |
| 20221 | aacaaagaga gttagaagta agaaaattca ggttatcagg ggcttgatat cttttttttg |
| 20281 | tgactgtagt caggttaccc aggcctttgt ggatcaattt ctcaaacaaa taacagagat |
| 20341 | gaaaacaact gtcagtagct actgtcacaa aaataatatg ataaacatct tttcgtttcc |
| 20401 | tatggctgcc ataactaatt atcacaaact tggtggctta aaataacaga aatgtatttt |
| 20461 | ctcacagctc cagagtccag aattccaaaa tggaagggtc agcaaggctg cacgccctca |
| 20521 | aaacactctt ggagagaatc ttttcttgct tcatccagct tctgatggct ctaggcaatc |
| 20581 | ccccgcttgt cactgcatca ctcoggtctc tgcccccatc ttcaaatgac cttcctctgt |
| 20641 | gtccctgtct tctcttctgt ctcttataag gacacttgtc attgtacagt atttagggtc |
| 20701 | cacatagata atccaggagg atctcatttt aagatcctta acttcattac atctgtacag |
| 20761 | atcctttgtc caaattaagg taatgttcac aggttctgtg ggttaggatg tggacatatc |
| 20821 | attttgaggc ccaccattca atgcactaca attttctttg taagtgttct gagaacccag |
| 20881 | gaagaataag taaatcctct gattcaatca aaaatgagca gaatgtaaac caaaaatact |
| 20941 | agaagatatc ctttttccta ggcactggca tacgtttcaa tagtatctct aattacagat |
| 21001 | gtatttacac ttctaagtgt cagaaatgaa gattattatg acaaatttaa aatttagtaa |
| 21061 | cttgttttta aactggtatt tttgtttttt taagcgatca tctcagtcca cattggaacg |
| 21121 | atctgaacag cagatcaggg ctgcttctag tttggaggaa ctacttcgaa ttactcactc |
| 21181 | tgaggactgg aagcbgtgga gatgcaggct gaggctcaaa agttttacca gtatggactc |
| 21241 | tcgctcagca tcccatcggt ccactaggtt tgcggcaact ttctatgaca ttgaaacact |
| 21301 | aaaaggtagg tgtgtggatg tggagacaaa ttctttctct ttattagata gtgacaaaca |
| 21361 | cacttacttt ctccaaaagt ctgcgtaggg cagacacgtg tcagagggag aatgaaagtc |
| 21421 | ggtctttgat ttccagtatt agtgcagaat cttgattttt taatgtttac ttcacagaga |
| 21481 | aatatattca atatgatttt gagataaaaa acacaaaaga acatctcaaa atatagaaat |
| 21541 | aaatttaact caaggatgtg ggggcaaaga aaacccagga agactattgt tctcaataga |
| 21601 | aaaacaatca tcaggtttac ttagaaaata aaacctgggg attcatccac aaggagtatt |
| 21661 | ctactgccat acatacatag acaaaaacca aagatggcca ggcatggtgg ctcatgcctg |
| 21721 | taatcccagc actttgggag gccaagaacg gcagatcgct tgagcccagg agtttgaaac |
| 21781 | cagcctgggc aacatgacga gaccccatct ctacaaaaaa atagaaaaaa atagctgaac |
| 21841 | attgtggtac gtgcctgtat tcccagctac ttgggaggct gaggtgggag gatcggctga |
| 21901 | gcccaggagg tcgaggctgc agtgagccat gatcatgcta ctgcactcca gcctgggcaa |
| 21961 | cagagcaaga tcctgtctca aaaaaaccac cccctccccg aaccaataga ttaggtatag |
| 22021 | cttttttttt tatttctgca aagttgtgtg attttttagt aaaagtgaaa agtaaaagtt |
| 22081 | agacactgag ttgtaatatt gggtactggt attgctagga gaataatgaa gggagaaaat |
| 22141 | tctcaagaca aaatgatcaa aactccagac tagaaaataa cataatttga aataaggata |
| 22201 | atgatcgttc cctagtaacg tatgaagatt ctttaaaaga aaatcaggcc aggtgtggtg |
| 22261 | gatgacacct gtaatcccag cactttggga ggttgaggca ggtggatcac ctgaggtcag |
| 22321 | gagttcgaga ccagcctgac caacatggtg aaaacccatc tctactaaaa atacaaaatt |
| 22381 | agccaggcat ggcggtgcat gcctgtaatc ccaactactt gggaggctga ggcaggagaa |
| 22441 | tcacttgaac ccaggaggcg gaggttgcag tgagccaaga ttgcaccatt gcactccaac |
| 22501 | ctgggcaaca agagtgaaac tccatcttaa aaaacaaaaa aacaaaacaa aacaaaacaa |
| 22561 | aaaaacaaag aaagaaagaa agaaaaccag tgtgagtagg tactgtcata atgaataaaa |
| 22621 | tgcttgtcaa gtaaagttct gtggtttctg attaggcact taattttttg atgtcatgtc |
| 22681 | aaaacaacac aagaagctgc tacagcattg ctgatctagc attctagatt tgattaacat |
| 22741 | gattacctgg taactgacta tatatatagc ataattcaaa ttaaccacta ggccaaaact |
| 22801 | atttttttct ttctacctaa aattgtattt gttttttccc atttgtaaaa agatgttggg |
| 22861 | gttgcacaga cctctgttgt tggcbgtggg gagagctgtg catagatggg gacatattct |
| 22921 | ttggtagcac cttgggcatg agtgtgaaga ggattcttag tgttcatgtg tatgactgtg |
| 22981 | tgtctaagtg agttgatgtg gtaggaacca tagcctcgag ctcacttcag acagaaattt |
| 23041 | gaagctctag atgtttctat gtggtgaatt aatatagcct tcatgagaac aaagggttgg |
| 23101 | cacctcatct agaggtcaaa ggccttaacc agattacctg actgaaggcc tgaggtgaac |
| 23161 | ctggagctat ttcatgaaca tgaacgttac cataaaacca agactaggtg atctaaaaaa |
| 23221 | agtcaatgac attgacattt tctaaggaga aaaccattaa aatatcacgg aatttgcaaa |
| 23281 | ccctatgaat ttatctcgta atttctaggg gatcatctga agttatttac taagttagaa |
| 23341 | gcagtcaata atgaaatcag catgttacac caattagaat ttgttatcaa ctctcccttg |
| 23401 | tggatttcaa ggtaatttcc tgaggcagag cagtcctgct gggaagcagt aaagaatagt |
| 23461 | agagttatga agcctttaat cagtgatgtt ggtcataatt gcatatgatt ttgattaaaa |
| 23521 | gcttaccact ccaatttcaa aacttagttt tataaaaaga gaaaaagcaa atggaaagca |
| 23581 | acctttaagt agaattttaa attgggaata ttttcatagg ttggtttctt gttttttcaa |
| 23641 | ggattcatga ttaattatag tgaattaaat aataaccata ataatactta attgagtaat |
| 23701 | ttatctctca bitgttcagc cttcaggctt tttatataaa tattaataat cagaaatttt |
| 23761 | taaaaaggtt tcttgggggc tgggcacagt ggctcacgcc tttaatccca gcactttggg |
| 23821 | aggctgaggt gggcggatca caaggttaag agatcaagac cagcctggcc aacatggtga |
| 23881 | aactccgtct ctactaaaaa tacaaaaatt agctgggcat ggtggcgcat acctgtaatc |
| 23941 | ccagctactt gggaggctga ggcaggagaa tcgcttgaac ccaggaggtg gaggttgcag |
| 24001 | tgagccgaga tcgcgccgct gcactccagc ctggcaacag agtgagaccc cgtctcaaaa |
| 24061 | aaaaaaaaaa aagtttctca ggatagaatg ttttattatg aggaaaaaaa taaaaaaaaa |
| 24121 | caaagtttag gtagactgtg tagtataatg cagtgctcca gacaactcat attatgtttt |
| 24181 | cagtgattga agaagcaaca tatgaaaaaa attggtatta agggatgaaa aaggagaaag |
| 24241 | gaatggagaa gatgttataa gggaataatt tagggactag ggtcacatct caaatggtaa |
| 24301 | tattgtatca gtatacttca agcagacgtt ttctctattc cttctattcc cctatatggg |
| 24361 | aattcaggat aatctcagga gagttcttga aaatgcagta aaaataaagg agagtttgcc |
| 24421 | tattgttgca taacacatta ccacaaaact tagctactta aaacaatatt tatctcagtt |
| 24481 | tctgtgggcc agaaatgctg acatggccta gatcagtttt tctgcttcag atgtctctgg |
| 24541 | aggctgcagt taagatgttg gcttgggctg caggcatctt agggcacagc tgaagcagag |
| 24601 | tgtgctttca agttcagtca gctgttgttt gcaggactca attcctcaca ggctgttgga |
| 24661 | ctgagggtct tagttcctcg ctggctattg gccagaggct gcctcaagtt ccttgtcgtg |
| 24721 | tggacctttc taacatggcg gcaaactaac acaagaactg aaaaccaaac accgcatgtt |
| 24781 | ctcactcata agtgggagtt gaacaatgag aacacatata cacaaggagg ggaacatcac |
| 24841 | aaaccagggc ctgttggtgg gtggggggca aagggaggga gagcattagg acaaatacct |
| 24901 | aatgcatgcg gggcttgaaa cctagatgac gggttgacgg gtgcagcaaa ccaccgtggc |
| 24961 | acatgcatac ctgtgtaaca aacctgcacg ttctgcacat gtatcccaga acttaaagta |
| 25021 | aaataagcta aaaaaaaaaa aaaaaaaaaa agcctgcaag tggagaagga ggagagagag |
| 25081 | agagagagtg tgtgtgagaa agtgagacag agtaagaaag aagggagctt gcaataagta |
| 25141 | gaaacaagac tgcaatcttt tttaacttca tcatggaagt ggtatcccat cacttttgct |
| 25201 | gtattctctt cataagaagc aagttactag gttcagctca cacacgaggg gaaggcattt |
| 25261 | acacaagggc atgactatca ggagatgggg accactgggg gttagtttca aagctgccta |
| 25321 | ccacaagaga ttttcaccag caccacaacc ccacacataa taaataaact ctacagttcc |
| 25381 | atggcaagct acaaggaagt ctttgtgttg tctttatcat aaaattgaat ctttccccca |
| 25441 | aaacacctgt agaaaggcta tgcagcatta agtatcaaag ggtcaaaatc taatttaaaa |
| 25501 | aataaattat gagagaaaag caagtactgt tttacaagaa agtatataat ttttttattc |
| 25561 | ccataggctt ctttttttgt taccttcaaa ttaactatgg tcaaaggatt gctcattcca |
| 25621 | gtaataagaa caggcttatt tcatcattta cataaatatc agtatcgggc acttgcaaat |
| 25681 | gagaaaactc tgatttgagt aaggattagt ttcacttcaa tttccattta tgcgtttgcc |
| 25741 | aagtttaaat cctactgtag ttacttaagc aaccaacaga aacatatttc ttgatgatgc |
| 25801 | taaaacctac ctacacctgg ctttaaagca gaagaaatgc tatccagctg tatcctataa |
| 25861 | attgcttgat aaataatatt tacatgttat ttctttacac gcttctattt acttccctat |
| 25921 | taaccagctt ctccagatgt attcagtgta attatgtaca ggacatggtg ttctacctca |
| 25981 | aagggaaata aaatacttat attctacaag gtatggtata aatgggaaag agcacagaga |
| 26041 | ttggttgtaa cttctctgca aacaatcttt ggttaaagga ctgacaaaaa atatccatct |
| 26101 | agtaaaagaa acaagcagta gaaatgtgaa aatgactgtg ttcagcataa ccctggcaca |
| 26161 | tgttggcaca ccaggaccat tccacctgct agccccagtg agctttcttt ctctctgcag |
| 26221 | ttatagatga agaatggcaa agaactcagt gcagccctag agaaacgtgc gtggaggtgg |
| 26281 | ccagtgagct ggggaagagt accaacacat tcttcaagcc cccttgtgtg aacgtgttcc |
| 26341 | gatgtggtgg ctgttgcaat gaagagagcc ttatctgtat gaacaccagc acctcgtaca |
| 26401 | tttccaaaca ggtataagga catcggcaag cctgccgtct ctaaagccct gatgataata |
| 26461 | gtgttgatag ctacaacaac cttgttgcta aacactgtac taaatgcttt atgtgcataa |
| 26521 | tcgcatttaa tctttgaaac aaatcaagag ggtattttat gctcctttat agatgaggaa |
| 26581 | actgaggctc agagaggtaa cttacttatc caagtttaca cggctgataa atagcagagc |
| 26641 | tgggattcaa acccaggtct gtgggaggcc agagcctgaa ctcttatcag ttattgtctc |
| 26701 | tctgatgcta tgatgtcaag actcagaaaa ccatatggaa gttactagga caggatgtta |
| 26761 | aaaggaatga agatagctga tggcaggttc tgtgttgctc aaatggagtg attgagaaaa |
| 26821 | atttaatgaa ggttgtgggc agaattgggg aaagagggat gggacagtga agcacctgaa |
| 26881 | gctagcaaaa agggtgagat ttocccaccc ttcgactgag gagacaaggg gagggagcag |
| 26941 | ttatgggaac ccagagggaa agcggagaag agggtttctg gactggccac tgtcaactct |
| 27001 | tagcctggca gggagtaggg gcaggggctg ggagaggcaa gagggtttat agatgtagcc |
| 27061 | catggagggc agcgtcctgg ggcacagagc ctgatgggga aggaagggga gaggatggtg |
| 27121 | tctctggagg ggcaaagaga gaatatctag cacaaaagac atttggaaat aattctgcag |
| 27181 | tccccatgaa agaaataatt cagccctctt catcttataa atttcccttt gcttagtagg |
| 27241 | taagttttcc tttctctggt taccatgttt ttaataaatg tcgcatatga gagcagtagt |
| 27301 | aacaaacttg ggaaagcaat actaaaaaac tgtacatcaa actacattat ttttggtatc |
| 27361 | bittgcaaat tctttgaatg ttgatatact tatttaattg tcagtttgcc tatatggttt |
| 27421 | attctccttt taactgcaaa aagaggtagt acaagaaaat tacatgcact gaatatatcc |
| 27481 | atgttaggta attttcttaa tgctgatttg ttttgttaat agatttactt atccttaatt |
| 27541 | ttgttcctcc accctctaaa atatccttta aactgcagag tttctttttc taaattcttt |
| 27601 | tagagaatct tggagcttgt gtgtatttta acctctacta tttgacaatt aggaaaaaaa |
| 27661 | aatgacccaa gtagggctaa gcatcaattc tgagtagaaa aaaagcttgc ataacgtaag |
| 27721 | agggtttagt tcatgatcat gtgattccct agagcatagc caaaaaattg tgcaagaaga |
| 27781 | tgaagtttat gcaaagtaaa cactacaaaa acactctcac tgagactcat ggaagttcca |
| 27841 | ttacatattg agatgtcaga ataaacacaa ccttaatgga ttgcttccac ttaatagata |
| 27901 | gttgagtact ttattttgac tgctgtactc aacaaatgat taatttcata caatgtactt |
| 27961 | aagtgtaagt gtacagcagt ttttcttcca aatatgcatt tttgaacatt ttaatagtag |
| 28021 | ttttttaaaa actatctctc gccccaaagt tatggactat ttggcaaacc acctcttaga |
| 28081 | aacaaagtga acaaatgaat actctatgta gtaaggacta catggaatgg tgattgaata |
| 28141 | gacaatgcat taaaatcaac ttcaaactca tccacacatc atagaaccac tcaagaaagt |
| 28201 | tgtgtaatct ctctgtacat ctgtttcttc acctgtaaga tggggataat aaaaacccac |
| 28261 | cccataatgt tgttataagg ggtaaatgag ttaacatgaa agcacttaca acagggcctg |
| 28321 | gcctacggaa agcgtagaaa gaatgatata catgttagct ataattattt gtttgggcat |
| 28381 | gatgatcttg tatcagtcag ctattaatac acagtgctac gtaacaaaca cactaaaact |
| 28441 | cagtggttcc aaacaacaat gatttactct cacttatgta tctgaggttc agttagttta |
| 28501 | gggtggattc agcbjggctt gactctaagc tgcagtgtga gtccaagtca acttctatgt |
| 28561 | ttcccatcgc ttggaccagc aagcctgccg gattatgttc ttttcatggc tatagcataa |
| 28621 | gtttgagaaa acaagcccca taaaacatgc acagttcaaa tttgtacttc tgtcatgtct |
| 28681 | gctaacatcc cactagccaa agaaagtgac atggccaaat aaaaagtcaa gagacaggaa |
| 28741 | actatatttc ttcaatagag atggtgtgag aagggatgaa taaatagttt gaataataat |
| 28801 | ctattctaat aaaatcctaa agaaactgca tagctttaat ctcatttaac ccttacgatt |
| 28861 | tacactcaaa tttaaagcag gagcagggat taacagtacc attttacaag agtatcaatg |
| 28921 | atcaaaaagt taaaagattt gaccaaactt aaacacttca taaagttaga tgagcctgtt |
| 28981 | tagttccatt cttaatggac aacttggcta aagtcattgc cgtaaatgac aaaggcaaaa |
| 29041 | attcagacta gaacaacttt aaaaatgata tataaaatta aaaaaatata ttgtggttat |
| 29101 | ctttctctgc tgtagctctt tgagatatca gtgcctttga catcagtacc tgaattagtg |
| 29161 | ccbittaaag ttgccaatca tacaggttgt aagtgottgc caacagoccc ccgccatcca |
| 29221 | tactcaatta tcagaagatc catccagatc cctgaagaag atcggtaagc acttttgttt |
| 29281 | ttttttttat actggattat gcttattttt gccttgccaa aactgtgtgt agaggatttt |
| 29341 | tcattaatgt agagttatca atttatcttc aaattattca tattttagct tctaacctga |
| 29401 | acctactggt aataactggc aaatacatat ttttattaga tgttagatat atattgtggt |
| 29461 | aggaccaaaa cgctcagtgg tatgagcttg tcatgtctac ttgtagacac gtttgttctt |
| 29521 | aatatgcata gtctactgca ggtgtaaaac cgctgtaacc tcaaatttag cacatgttcc |
| 29581 | agaacaagca ttaaataaaa cttatttcag gcbgtttgta tatttacatc ctgaaatgag |
| 29641 | agaaaacaaa gaagattcct ggggtcattg gggagtgaca aaactagcag cacgtgagtt |
| 29701 | ttgcaacttt ctaatctttc ccccttaaga ttaccaaatg gaaaaccctt attttaaatt |
| 29761 | aaaatgactt aaaattaatt tggctttgaa attctcttga gattaatttt gtgattattt |
| 29821 | ataagacaaa cgacaaaacc aaaggtaagg atcacttatt tgaagaaaaa aaaggattta |
| 29881 | atataaagaa acaagtaaag ggtaactgaa tgaaaagatc attagttaga tacttagact |
| 29941 | ttgcaaaagt aaagcaataa atatattgtg tgagtggcaa gtaaaagagt aagctggtga |
| 30001 | ttagttaaca ttgaaccaag aagacacctc ccgcctctct ccagcagaaa agaaaacttg |
| 30061 | tagaaaagca acatttattc tcaagatgtt cctgcbgttc tggccaatgc ttttatcttt |
| 30121 | attttaaaga aaataatctt aggggccggg cacggtggct cacacctgta atcccagcag |
| 30181 | tatgggaggc tgaggcgggc agatcacgag gtcaggagat tgagaccatc ctggctaaca |
| 30241 | cggtgcaact ccgtctctac taaaaatata aaaaaaaatt agccgggtgt ggtggtgggt |
| 30301 | gcctgtagtc ccagttactt gggaggctga ggcaggagaa tggcatgaac ctgggaggtg |
| 30361 | gagcttgcag tgagccgaga tcgcgccact gcactccagc ctgggcaaca gagggagact |
| 30421 | ccgtcaaaaa aaaaagaaaa gaaaataatc ttaggaggta tgttagaaca tatttaattt |
| 30481 | tcgtaaataa actgttgaca gctataattt gcatagtttt aacatatacc ttcttaagaa |
| 30541 | gattacaatt tccaaaatta cagtttagtt tctaaacatt tccacgtaga atatttataa |
| 30601 | ttacagaatc actatctagt aagatgcagt tcataaatac aaattaagct cctttaaaat |
| 30661 | cttttttttt tttgagacgg agtttcgctc ttgttgcccc ggctggagtg caatggcgag |
| 30721 | atctojgctc actgcaacct ccgcctccca ggttcaagtg attctcctgc ctcagccttc |
| 30781 | caagtagctg ggattacagg catgtgccgc cacgcccggc taattttgta tttttcagta |
| 30841 | gagatggggt ttctccatat tagtcaggct ggtctcgaac tcccgacctc aggtgatctg |
| 30901 | cccgcctcgg actcccaaag tgctgggatt acaggcgtga gccaccgcac ctggcccgct |
| 30961 | cctttaaaat cttaactctt ctcacatcct tatcattttg tggtgagaac atttgaaatt |
| 31021 | tactctctta gcaatttgga aatataaaat acattattat tattgactat agttaccatg |
| 31081 | ctgtgcaata gatctcaaaa ccttatttct ccaaactgag tataaaaagc aaaaaataaa |
| 31141 | tttaaaaaat cttaactatt atctactcag gaatttattt aatgttccaa agttttgatt |
| 31201 | atccttgaaa gcttttaaat ctggttcatt gttttctttt cctagcbgtt cccattccaa |
| 31261 | gaaactctgt cctattgaca tgctatggga tagcaacaaa tgtaaatgtg ttttgcagga |
| 31321 | ggaaaatcca cttgctggaa cagaaggtaa tgatagtagt agtaaaataa taacagtaat |
| 31381 | aataaaagta gcaacaatag cagcaaccgt tattgagtac ttagtatgac cagacaatgt |
| 31441 | tgtaaatott gtatatgttt taactcattt aattctcaca acaaccctgt aatgtactat |
| 31501 | tgttatttct ttttttacaa ttacaaaatg ggcacaccaa gatatcgagt aatttcctca |
| 31561 | aggtcacaca gctgagagtg atagaagtgg tcagattcca gaaccctctg taccataatc |
| 31621 | atatagtaag tccatcaatg catcactctg ctttttaatc tcaaagaaaa gcccagagtg |
| 31681 | gttggagcat cctgaaacac tgaaattgaa aggggatgtt aaacttgaat gaaagctgct |
| 31741 | tcagaactaa atactagtca actgcagttt actaatttat ttgtataaaa gtctcacagg |
| 31801 | aacataaagt aattttggac ttggtttctg tcagctgcat catatgaaaa cacgccagta |
| 31861 | atttgaaaaa agaaatggag aataagagaa aatttttctg gaattagggc aaagcaaaag |
| 31921 | caaaatatgg ctattggtct aaggggagct gggagcaaaa tgagatatag tcaaattgtc |
| 31981 | attgctcata atccgtaatt ctgtgacttt gggtgatgaa catttgcttg aaaatgaatg |
| 32041 | cattttaatg tattttccag agagctttac atggttccac caaaaaaaaa aaagaaagaa |
| 32101 | aaaaaaagtc acaagtagtc atgcagtttt aaccttgctt gttttaaact ataatctata |
| 32161 | agcaagaaca gtaagggcta aactctaaga aatctgtcct ataaactgta aatagcaacc |
| 32221 | tactctccat tcttctgcct ctcagcttcc ttctaaatgt tttcacgaaa gctcagccca |
| 32281 | gagcatgtgg ggaaatttaa tatttatgtg gtttaaatgc ccatgccgat cttgaatgtt |
| 32341 | ctttacaccc ttcccaccaa gtgttcagcc atcctctacc agaacataca tcagttattt |
| 32401 | ggagactcgc catccagtga ggcagcacca aggggtgggc acactcctgt tgttatactg |
| 32461 | aaatgaaatc tctctccaga acagaccaga ctgcccaatg gaatacaata agagtcatat |
| 32521 | atgtaactca cattttacat taaaaagata aaaagaaaca agtgaaattt atcttaatat |
| 32581 | attttattta atccaatcta tccaatgtac tatcatttca acatgcaatc aatataaaaa |
| 32641 | atttatgagc tagcctacat tctttgtgct aagtctttga aatccagtgt gtgttgtaca |
| 32701 | cttacagcac atctcaattt agactctcta tttcatgccc aatagtcaca tatggcccat |
| 32761 | agctaccttg ttggacagcc cagctcaaga gcatcaactc attagttcag gttttaccct |
| 32821 | atagaaccat gtaaaacaaa tctaattctt tctccatgtg actcatgttc taaaagtgtt |
| 32881 | agaaaatttt tgtggggttg gccgggaatg gtggctcatg cctgtaatcc tagcactttg |
| 32941 | ggaggccgag atgggaggat cacttgaggt tgggagtttg aaaaccagcc gggccggccg |
| 33001 | ggcgcggtgg ctcacgcctg tagtcccagc actttgggag gccgaggcgg gcggatcacg |
| 33061 | aggtcaggag attgagacca tcctggctaa aacggtgaaa ccccgtctct actaaaaata |
| 33121 | caaaaaaaaa attagccggg cgtagtggcg ggcgcctgta gtcccagcta cttgggaggc |
| 33181 | tgaggcagga gaatggcgtg aacccgggag gcggagcttg cagtgagccg agatcccgcc |
| 33241 | actgcactcc agcctgggca acagagcgag actccgtctc aaaaaaagaa aaaaaaaaaa |
| 33301 | aagaaaacca gccgggccaa catggcgaaa tcccatctct actactacta aaaaaaaaaa |
| 33361 | aaaaaaaaaa aaaaaaaaaa aaaaaatagc tgggcatggt gatgtgcgcc tgtagtccca |
| 33421 | gctactcagg aggctgaggc atgagaatct cttgaacctg ggaggcagag gttgcagtga |
| 33481 | gccgaggtca tgccaccgca ctcgggcctg gatgacagag tgagactctg cctccaaaaa |
| 33541 | aagaaagaaa gaaagaaaga gagaaagaga aaaagaaaag aaaagaaaga gagagagaga |
| 33601 | gaaagaaaga aagaaaagaa aagaaagaga gagaaagaaa gaaagaaaag aaagaaaaag |
| 33661 | aaaggaagga aggaaggata gaaagaaaga aagagaaaga aagtaagtta ctgtgggaag |
| 33721 | ggcaatatac ttagagctta aaggctctag ctgaggattt cctggcacaa agcctctgag |
| 33781 | acagttttgt acggaggagg tttgtaggca ggcacaaggc attaaagggt aaagagagag |
| 33841 | gcagcagacc acagcgcagt tgctaagagg catcagccta agccaatggg aagttgaagc |
| 33901 | tgggaacccc tgcgggggta agtcttaaat ctaggtgaag aggctgagcc ctgtagaccg |
| 33961 | ttcattggct gccttggaga ggggatgtag ccttaggaga ggcagcttat ttctgctgga |
| 34021 | gataattctt gggaaggcac tcagccagta cacgtgtgta gagggtggta agaagaatag |
| 34081 | ggaagaagag aagtgctgtt ctaagcagcg ggtatatgag cactcagggc tggaggtgag |
| 34141 | agaacatact ggaagaagtt cagcatggtg gggatataat gggtggggac ataatgggga |
| 34201 | aggggctggt tagatgtgag agaagaagct ggacaggctc acaggggcaa agcagaagga |
| 34261 | ccttgtaatt cttgcatggg agcttggatg tgatactaag gacaaaggga aaccacacaa |
| 34321 | ctggctaaag caggagagtg acagtaccca ttagaatttt aacaaaatca tatcagcaac |
| 34381 | agtgtagaag aggatgtgtt gactcggggc agggaaggag gcaggaaacc ttttgggaag |
| 34441 | ttgtagcaat gatgtaagtg agaggcgcag ggtggactgg atcacaattg tttctcattc |
| 34501 | tcatctccta taagtcccca atgtggatca agttcacatt catttctcca gtggccccat |
| 34561 | caaattgatg ggatttactg agcttactgc tcactatact ttttttccgc acatgtgact |
| 34621 | gttaagccaa ctgccttttc tacagtgact tctcttgccc agatgagcac atttaggtta |
| 34681 | gtgaatccaa gtctaattta attgagtctt gttagagtca ggtcattgct ctaaattgtt |
| 34741 | aaaacaaacg atatacacaa acacacacac acacaaaaga ataaattgag gaggtcccaa |
| 34801 | ttctgtattt taaaatactt aatatatccc ttccttcttg aagagagcta caatttcaag |
| 34861 | aatttatttt gaaaaaattc tatcagttta ttgataaaaa tggtaaaagg aacaaagctg |
| 34921 | agaacagaac atatcatcag actctctagg gacggaaatc aatacataat acttcaagat |
| 34981 | atcaccctca gctacttgct gttccaccac cagttctctt gcatccagtt tttgtttctt |
| 35041 | ttaacttggc tataaggatg tgagcacaga ctctgccaaa cagcctgctg aagttctgca |
| 35101 | aacaccttgt ggattgtgtt tccctgaaca accctgtcca acaatggcct tgggcctgtc |
| 35161 | tgaagtattc ttagtgaact tctgctgcca cctagtgatc attgcgaatt tttaaaaagc |
| 35221 | ctctttatcc tttaacaagt tactcttgtc ttaccccaaa gcggcatctg cctcactggc |
| 35281 | atatgactta cagagctcac attttccctc tccacaaagt tcaaaatgga gacttctcag |
| 35341 | atttagcctt ccagcaccca ttctcaaacg tcgtgcatta cagaaaagtt taaactataa |
| 35401 | agttgaaccg cacgagattc tcattttgat atgtcattaa agtcagatat tggcaagaac |
| 35461 | atatggttta acccaatggc ttggaatgct ggatgggatt cccagaagct ccatacatcg |
| 35521 | gcctgcttca ggaccctttt attacaccaa ataaacccaa caaacatcca tcgagtgcct |
| 35581 | attttatatc agaacaatga gacctgctcc ttctccttca ggacatttta cttaatgggg |
| 35641 | gaaacataca agtaaacaga caaatgtaat cagtgcaaga aagaatcgga cagcaatatg |
| 35701 | cacaggataa ggacacttag ctaaacctgc agacattggg aaagatgtca gggaaaaggg |
| 35761 | atggcagaag gaaagacaag gccaaaggca tgggactggg aggcactgga atatcagggc |
| 35821 | aacagaaagt gctgtcattt ggtataaatg gaacacacag gatatatgaa agcaagaaac |
| 35881 | agaagatgag tctgtgaggt tactatgatc cagttcatgg agggccttgt atgtcctggt |
| 35941 | gggaaaattg gtttttattg tgggaaggga accttttaat gcattcaaag cagtggaatg |
| 36001 | aagtgatttg gtttgcattt gttaatcatg tgaagaagaa taggttggag ggataaaact |
| 36061 | tgtggtggtg agatgagtga cagagcgaca gccatctaca aaagaaacac aaggtacaga |
| 36121 | ctgaattgtg ggagtggtag tggggatgga aaataaagaa caaatttaga gaggcagaac |
| 36181 | tgagtggaca tgatgctcag acttaccaga gcaggtgagt caagaatgat gtcaccattc |
| 36241 | cctaaatgag gcaaactggg agagaagcag atttggggag aaaactacta aacatgtacg |
| 36301 | tccctccaaa accttcaaga atgctaaagg gacagagtca agttcttcca aagtttctgt |
| 36361 | ctcttccatt tattttgcaa tgaatttcaa cttgttaatc agaaatggat ttggagtagt |
| 36421 | agctattcaa ttgttttata caccttctag aagaaatatt tgtcagatga gtatgtcaaa |
| 36481 | aacttctcag accctcccct ttacttgaat gtatcttcca gcagatgttg gtggaatcaa |
| 36541 | agttcattat tactcctata tcttttatac tagtgttatg atctgcagtg aggacatgca |
| 36601 | ttgctattgc tacgcctggc ctggaatgtc tgaggtgtat ttctacaaca ccaattattt |
| 36661 | tgtgtgtctc tccttttaat ctccaccaaa aaatggagac agctagattc ataaccctta |
| 36721 | gacatcctat attttattac taatacttgg ttgtggaagt ttgcattcta aacaagtcat |
| 36781 | caacataaaa atctctaaaa atattttatg aaaaatattc agcttatgat attttaaaaa |
| 36841 | tcacaaacct aagaaaacac acaaacctgt gtttgtattg tgaaatcaga ggttaatctt |
| 36901 | cctcaccctg tgaaaaaatt aggggcataa atctatagga aggagattgg gtgaatccaa |
| 36961 | gttacttacg gatgacataa aagatacaat caactttggt gatccaattg ccacactaca |
| 37021 | actatctgac caacacgttt ctgtttctct ttagaccact ctcatctcca ggaaccagct |
| 37081 | ctctgtgggc cacacatgat gtttgacgaa gatcgttgcg agtgtgtctg taaaacacca |
| 37141 | tgtcccaaag atctaatcca gcaccccaaa aactgcagtt gctttgagtg caaagaaagt |
| 37201 | ctggagacct gctgccagaa gcacaagcta tttcacccag acacctgcag gtaaatgcct |
| 37261 | tgagttgtct tgcctttact catgacattt aacgtagatg ccattttaat ttggtgggat |
| 37321 | tccttagtaa gctaattttt ccccttggtg cctgagtccc ttttggataa tagaacagag |
| 37381 | gtttagcagc taaggaataa gcaagagtat accgagtcat gattaatcat gaaaataaga |
| 37441 | aaatcaatcc tgcctgttat tatgtgtgta gagctttttt tttttagacg gagtctcgct |
| 37501 | ctgtctccag gctggagtgc agtggcgtga tctgggctca ctgcaacctc cgtctctctg |
| 37561 | gttcaaggga gtctcctgcc tcagcctccc aagtagctgg gattacaggc acgtgccacc |
| 37621 | acacccagct aatttttgta tttcagtaga gacggggttt caccatgttg gccaggatag |
| 37681 | tcttgatctc ctgacctcat gatccacccg tctojgcctc ccaaagtgct gggattacag |
| 37741 | gcatgagcca ccgcgcccac cojgggttta gagctcttaa acaacaatga aattggtaaa |
| 37801 | atttaagcag ttattaggat attgcttaat aataatattt taggaagggg aggtttcaga |
| 37861 | tcatcaacct gtttagttca atctctcatt gtttatgtct atatatgttt gtttttttct |
| 37921 | ttaatggagg gaatatcatt ggatttatat agcaagtaat ttattcatga atagaacact |
| 37981 | tggaggttac tgattcactt cagtagattt tgaatccatg tgggataacc atgcaggatt |
| 38041 | ttatactgac attttaaata catgtctgga aaacttaatc aaatcagagc agagttattc |
| 38101 | ttttgaatcc agtcattgat tctgaattct catctttatt ttcttttgca acagctgtga |
| 38161 | ggacagatgc ccctttcata ccagaccatg tgcaagtggc aaaacagcat gtgcaaagca |
| 38221 | ttgccgcttt ccaaaggaga aaagggctgc ccaggggccc cacagccgaa agaatccttg |
| 38281 | attcagcgtt ccaagttocc catocctgtc atttttaaca gcatgctgct ttgccaagtt |
| 38341 | gcbgtcactg tttttttccc aggtgttaaa aaaaaaatcc attttacaca gcaccacagt |
| 38401 | gaatccagac caaccttcca ttcacaccag ctaaggagtc cctggttcat tgatggatgt |
| 38461 | cttctagctg cagatgcctc tgcgcaccaa ggaatggaga ggaggggacc catgtaatcc |
| 38521 | ttttgtttag ttttgttttt gttttttggt gaatgagaaa ggtgtgctgg tcatggaatg |
| 38581 | gcaggtgtca tatgactgat tactcagagc agatgaggaa aactgtagtc tctgagtcct |
| 38641 | ttgctaatcg caactcttgt gaattattct gattcttttt tatgcagaat ttgattcgta |
| 38701 | tgatcagtac tgactttctg attactgtcc agcttatagt cttccagttt aatgaactac |
| 38761 | catctgatgt ttcatattta agtgtattta aagaaaataa acaccattat tcaagccata |
| 38821 | taa |

In some embodiments of any of the vectors described herein, the vector includes a sequence encoding an antibody or antigen-binding antibody fragment that can bind specifically to VEGF. In some embodiments, an antibody or antigen-binding antibody fragment can include a human IgG Fc domain (e.g., SEQ ID NO: 113). In some embodiments, the sequence encoding an antibody or antigen-binding fragment can further include a sequence encoding a secretion signal sequence (e.g., SEQ ID NO: 92). In some embodiments of any of the vectors described herein, the vector comprises a sequence of a T2A peptidase (e.g., SEQ ID NO: 99).

In some embodiments, the antibody can be a humanized antibody, a chimeric antibody, or a multivalent antibody. In some embodiments, an antibody or an antigen-binding antibody fragment can be a scFv-Fc, a V_{H}H domain, a V_{NAR} domain, a (scFv)₂, a minibody, or a BiTE. In some embodiments, an antibody or an antigen-binding antibody fragment can be a DVD-Ig, and a dual-affinity re-targeting antibody (DART), a triomab, kih IgG with a common LC, a crossmab, an ortho-Fab IgG, a 2-in-1-IgG, IgG-ScFv, scFv₂-Fc, a bi-nanobody, tanden antibody, a DART-Fc, a scFv-HAS-scFv, DNL-Fab3, DAF (two-in-one or four-in-one), DutaMab, DT-IgG, knobs-in-holes common LC, knobs-in-holes assembly, charge pair antibody, Fab-arm exchange antibody, SEEDbody, Triomab, LUZ-Y, Fcab, kλ-body, orthogonal Fab, DVD-IgG, IgG(H)-scFv, scFv-(H)IgG, IgG(L)-scFv, scFv-(L)-IgG, IgG (L,H)-Fc, IgG(H)-V, V(H)-IgG, IgG(L)-V, V(L)-IgG, KIH IgG-scFab, 2scFv-IgG, IgG-2scFv, scFv4-Ig, Zybody, DVI-IgG, nanobody, nanobody-HSA, a diabody, a TandAb, scDiabody, scDiabody-CH3, Diabody-CH3, Triple Body, miniantibody, minibody, TriBi minibody, scFv-CH3 MIL Fab-scFv, scFv-CH-CL-scFv, F(ab′)₂-scFV₂, scFv-KIH, Fab-scFv-Fc, tetravalent HCAb, scDiabody-Fc, diabody-Fc, tandem scFv-Fc, intrabody, dock and lock bispecific antibody, ImmTAC, HSAbody, scDiabody-HAS, tandem scFv, IgG-IgG, Cov-X-Body, and scFv1-PEG-scFv2.

Additional examples of an antibody or an antigen-binding antibody fragment include an Fv fragment, a Fab fragment, a F(ab′)2 fragment, and a Fab′ fragment. Additional examples of an antibody or an antigen-binding antibody fragment include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

Any of the antibodies or antigen-binding antibody fragments described herein can bind specifically to VEGF.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

A “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of antibodies and antigen-binding antibody fragments are known in the art.

In some embodiments, any of the antibodies or antigen-binding antibody fragments described herein has a dissociation constant (KO of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹¹ M, less than 0.5×10⁻¹¹ M, or less than 1×10⁻¹² M), e.g., as measured in phosphate buffered saline using surface plasmon resonance (SPR) for a VEGF protein (e.g., any of the VEGF proteins described herein, e.g., one or more of mature human VEGF-A, mature human VEGF-B, mature human VEGF-C, and mature human VEGF-D).

In some embodiments, any of the antibodies or antigen-binding antibody fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR), for a VEGF protein (e.g., any of the VEGF proteins described herein, e.g., one or more of mature human VEGF-A, mature human VEGF-B, mature human VEGF-C, and mature human VEGF-D).

A variety of different methods known in the art can be used to determine the K_{D} values of any of the antibodies or antigen-binding antibody fragments described herein (e.g., an electrophoretic mobility shift assay, a filter binding assay, surface plasmon resonance, and a biomolecular binding kinetics assay, etc.).

In some embodiments of any of the antibodies and/or antigen-binding antibody fragments described herein, the half-life of the antibody and/or the antigen-binding antibody fragment in a subject (e.g., a human) is decreased about 0.5-fold to about 4-fold (e.g., about 0.5-fold to about 3.5-fold, about 0.5-fold to about 3-fold, about 0.5-fold to about 2.5-fold, about 0.5-fold to about 2-fold, about 0.5-fold to about 1.5-fold, about 0.5-fold to about 1-fold, about 1-fold to about 4-fold, about 1-fold to about 3.5-fold, about 1-fold to about 3-fold, about 1-fold to about 2.5-fold, about 1-fold to about 2-fold, about 1.5-fold to about 4-fold, about 1.5-fold to about 3.5-fold, about 1.5-fold to about 3-fold, about 1.5-fold to about 2.5-fold, about 1.5-fold to about 2-fold, about 2-fold to about 4-fold, about 2-fold to about 3.5-fold, about 2-fold to about 3-fold, about 2-fold to about 2.5-fold, about 2.5-fold to about 4-fold, about 2.5-fold to about 3.5-fold, about 2.5-fold to about 3-fold, about 3-fold to about 4-fold, about 3-fold to about 3.5-fold, or about 3.5-fold to about 4-fold) as compared to the half-life of a control antibody and/or a control antigen-binding antibody fragment (e.g., any of the control antibodies and control antigen-binding antibody fragments described herein) in a similar subject. See, e.g., Leabman et al., MAbs. 5(6): 896-903, 2013. In some embodiments, an antibody or antigen-binding antibody fragment described herein has one or more amino acid substitutions in the Fc region that decrease its half-life in a mammal, and a control antibody lacks at least one (e.g., lacks all) of these one or more amino acid substitutions in the Fc region.

In some examples of any of the antibodies and antigen-binding fragments thereof described herein, the antibody and antigen-binding fragment can bind to a VEGF antigen (e.g., any of the exemplary VEGF proteins described herein, e.g., one or more of mature human VEGF-A, mature human VEGF-B, mature human VEGF-C, and mature human VEGF-D) (e.g., any of the binding affinities described herein).

In some embodiments described herein, an antibody or antigen-binding antibody fragment can decrease an activity of a VEGF (e.g., one or more of any of the exemplary VEGF proteins described herein, e.g., one or more of mature human VEGF-A, mature human VEGF-B, mature human VEGF-C, and mature human VEGF-D). In some embodiments, an antibody or antigen-binding antibody fragment can block a VEGF (e.g., one or more of any of the exemplary VEGF proteins described herein, e.g., one or more of mature human VEGF-A, mature human VEGF-B, mature human VEGF-C, and mature human VEGF-D) from binding to one or more of its receptors (e.g., one or more VEGF receptors) See, e.g., WO 1998/045331, U.S. Pat. No. 9,079,953, US 2015/0147317, US 2016/0289314, Plotkin et al., Otology & Neurotology 33: 1046-1052 (2012); and Ferrara et al. (2005) Biochem Biophys Res Commun 333(2): 328-335. In some embodiments, an antibody or antigen-binding antibody can decrease downstream signaling (e.g., signaling downstream of a VEGF receptor, e.g., one or more of any of the exemplary VEGF receptors described herein, e.g., one or more of human VEGFR-1 (SEQ ID NO: 111), human VEGFR-2 (SEQ ID NO: 112), and human VEGFR-3). In some embodiments, a decrease in an activity of a VEGF can be detected indirectly, e.g., through an increase in hearing (e.g., a 1% to about 400% increase (or any of the subranges of this range described herein) in hearing) or a decrease (e.g., a 1% to 99%, a 1% to 95%, a 1% to 90%, a 1% to 85%, a 1% to 80%, a 1% to 75%, a 1% to 70%, a 1% to 65%, a 1% to 60%, a 1% to 55%, a 1% to 50%, a 1% to 45%, a 1% to 40%, a 1% to 35%, a 1% to 30%, a 1% to 25%, a 1% to 20%, a 1% to 15%, a 1% to 10%, a 1% to 5%, a 5% to 99%, a 5% to 95%, a 5% to 90%, a 5% to 85%, a 5% to 80%, a 5% to 75%, a 5% to 70%, a 5% to 65%, a 5% to 60%, a 5% to 55%, a 5% to 50%, a 5% to 45%, a 5% to 40%, a 5% to 35%, a 5% to 30%, a 5% to 25%, a 5% to 20%, a 5% to 15%, a 5% to 10%, a 10% to 99%, a 10% to 95%, a 10% to 90%, a 10% to 85%, a 10% to 80%, a 10% to 75%, a 10% to 70%, a 10% to 65%, a 10% to 60%, a 10% to 55%, a 10% to 50%, a 10% to 45%, a 10% to 40%, a 10% to 35%, a 10% to 30%, a 10% to 25%, a 10% to 20%, a 10% to 15%, a 15% to 99%, a 15% to 95%, a 15% to 90%, a 15% to 85%, a 15% to 80%, a 15% to 75%, a 15% to 70%, a 15% to 65%, a 15% to 60%, a 15% to 55%, a 15% to 50%, a 15% to 45%, a 15% to 40%, a 15% to 35%, a 15% to 30%, a 15% to 25%, a 15% to 20%, a 20% to 99%, a 20% to 95%, a 20% to 90%, a 20% to 85%, a 20% to 80%, a 20% to 75%, a 20% to 70%, a 20% to 65%, a 20% to 60%, a 20% to 55%, a 20% to 50%, a 20% to 45%, a 20% to 40%, a 20% to 35%, a 20% to 30%, a 20% to 25%, a 25% to 99%, a 25% to 95%, a 25% to 90%, a 25% to 85%, a 25% to 80%, a 25% to 75%, a 25% to 70%, a 25% to 65%, a 25% to 60%, a 25% to 55%, a 25% to 50%, a 25% to 45%, a 25% to 40%, a 25% to 35%, a 25% to 30%, a 30% to 99%, a 30% to 95%, a 30% to 90%, a 30% to 85%, a 30% to 80%, a 30% to 75%, a 30% to 70%, a 30% to 65%, a 30% to 60%, a 30% to 55%, a 30% to 50%, a 30% to 45%, a 30% to 40%, a 30% to 35%, a 35% to 99%, a 35% to 95%, a 35% to 90%, a 35% to 85%, a 35% to 80%, a 35% to 75%, a 35% to 70%, a 35% to 65%, a 35% to 60%, a 35% to 55%, a 35% to 50%, a 35% to 45%, a 35% to 40%, a 40% to 99%, a 40% to 95%, a 40% to 90%, a 40% to 85%, a 40% to 80%, a 40% to 75%, a 40% to 70%, a 40% to 65%, a 40% to 60%, a 40% to 55%, a 40% to 50%, a 40% to 45%, a 45% to 99%, a 45% to 95%, a 45% to 90%, a 45% to 85%, a 45% to 80%, a 45% to 75%, a 45% to 70%, a 45% to 65%, a 45% to 60%, a 45% to 55%, a 45% to 50%, a 50% to 99%, a 50% to 95%, a 50% to 90%, a 50% to 85%, a 50% to 80%, a 50% to 75%, a 50% to 70%, a 50% to 65%, a 50% to 60%, a 50% to 55%, a 55% to 99%, a 55% to 95%, a 55% to 90%, a 55% to 85%, a 55% to 80%, a 55% to 75%, a 55% to 70%, a 55% to 65%, a 55% to 60%, a 60% to 99%, a 60% to 95%, a 60% to 90%, a 60% to 85%, a 60% to 80%, a 60% to 75%, a 60% to 70%, a 60% to 65%, a 65% to 99%, a 65% to 95%, a 65% to 90%, a 65% to 85%, a 65% to 80%, a 65% to 75%, a 65% to 70%, a 70% to 99%, a 70% to 95%, a 70% to 90%, a 70% to 85%, a 70% to 80%, a 70% to 75%, a 75% to 99%, a 75% to 95%, a 75% to 90%, a 75% to 85%, a 75% to 80%, a 80% to 99%, a 80% to 95%, a 80% to 90%, a 80% to 85%, a 85% to 99%, a 85% to 95%, a 85% to 90%, a 90% to 99%, a 90% to 95%, or a 95% to 99% decrease) in the size or the severity of one or more symptoms of an acoustic neuroma, vestibular schwannoma, or neurofibromatosis type II in a mammal as compared to the level of hearing or size of an acoustic neuroma, vestibular schwannoma, or neurofibromatosis type II in the mammal, respectively, before administration of any of the AAV vectors described herein. In some embodiments, a decrease in a VEGF activity can be detected in an in vitro assay.

In some embodiments, the antibody that specifically binds to a VEGF is bevacizumab (Avastatin®) (SEQ ID NO: 103) or an antigen-binding fragment thereof. Bevacizumab (full size antibody ˜150 kDa) inhibits all isoforms of VEGF-A. Bevacizumab received Food and Drug administration (FDA) approval in 2004 for colon cancer for intravenous (IV) dose of 4.0-7.5 mg/kg at 2-3 weeks (plasmatic half life 21 days), for intravetrial (IVT) dose 1.25 mg in 0.05 mL (half-life 5.6 days). Bevacizumab has a K_{D} for VEGF 165 (VEGF-A) of 58 pM. See, e.g., WO 2017/050825. The leader sequence of bevacizumab is SEQ ID NO: 104, the heavy chain variable domain of bevacizumab is SEQ ID NO: 105, the heavy chain constant region of bevacizumab is SEQ ID NO: 106, the light chain variable domain of devacizumab is SEQ ID NO: 107, and the light chain constant region of bevacizumab is SEQ ID NO: 108. An exemplary cDNA sequence encoding bevacizumab is SEQ ID NO: 109.

In some embodiments, the antibody that specifically binds to a VEGF is ranibizumab (Lucentis®) (SEQ ID NO: 95), or an antigen-binding fragment thereof. Ranibizumab (˜50 kDa) inhibits all isoforms of VEGF-A. Ranibizumab received FDA approval in 2006 for ocular use for intravenous (IV) dose of 4.0-7.5 mg/kg at 2-3 weeks (plasma half life of 0.5 days), for intravetrial (IVT) dose 0.5 mg in 0.05 mL (half-life of 3.2 days). Ranibizumab has a K_{D} for VEGF 165 (VEGF-A) of 46 pM. See, e.g., WO 2014/178078. The leader sequence of ranibizumab has a sequence of SEQ ID NO: 96, the heavy chain variable domain of ranibizumab has a sequence of SEQ ID NO: 97, the heavy chain constant region of ranibizumab has a sequence of SEQ ID NO: 98, the light chain variable domain of ranibizumab has a sequence of SE QID NO: 100, the light chain contant region of ranibizumab has a sequence of SEQ ID NO: 101. An exemplary cDNA sequence encoding ranibizumab is SEQ ID NO: 102.

In some embodiments, the antibody that specifically binds to VEGF is sevacizumab (APX003/SIM-BD0801), or an antigen-binding fragment thereof.

In some embodiments, the antibody or antigen-binding fragment thereof that specifically binds to VEGF is aflibercept (SEQ ID NO: 110). An exemplary cDNA sequence encoding aflibercept is SEQ ID NO: 114.

In some embodiments of the antibodies that specifically bind to VEGF and antigen-binding fragments thereof described herein, the antibody or antigen-binding fragments thereof includes a variable light chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable light chain domain of bevacizumab, and/or includes a variable heavy chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable heavy chain domain of bevacizumab.

In some embodiments of the antibodies that specifically bind to VEGF and antigen-binding fragments thereof described herein, the antibody or antigen-binding fragments thereof includes a variable light chain domain that is or includes the variable light chain domain of bevacizumab, and/or a variable heavy chain domain that is or includes the variable heavy chain domain of bevacizumab. In some embodiments of the antibodies that specifically bind to VEGF and antigen-binding fragments thereof described herein, the antibody or antigen-binding fragments thereof includes a variable light chain domain that is or includes the sequence of variable light chain domain of bevacizumab, except that it includes one, two, three, four, five, six, seven, eight, nine, ten, eleven, twelve, thirteen, fourteen, or fifteen amino acid substitutions, and/or includes a variable heavy chain domain that is or includes the sequence of variable heavy chain of bevacizumab, except that it includes one, two, three, four, five, six, seven, eight, nine, ten, eleven, twelve, thirteen, fourteen, or fifteen amino acid substitutions. In some embodiments the first antigen-binding domain includes the three CDRs in the light chain variable domain of bevacizumab, and/or the three CDRs in the heavy chain variable domain of bevacizumab.

In some embodiments of the antibodies that specifically bind to VEGF and antigen-binding fragments thereof described herein, the antibody or antigen-binding fragments thereof includes a variable light chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable light chain domain of ranibizumab, and/or includes a variable heavy chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable heavy chain domain of ranibizumab.

In some embodiments of the antibodies that specifically bind to VEGF and antigen-binding fragments thereof described herein, the antibody or antigen-binding fragments thereof includes a variable light chain domain that is or includes the variable light chain domain of ranibizumab, and/or a variable heavy chain domain that is or includes the variable heavy chain domain of ranibizumab. In some embodiments of the antibodies that specifically bind to VEGF and antigen-binding fragments thereof described herein, the antibody or antigen-binding fragments thereof includes a variable light chain domain that is or includes the sequence of variable light chain domain of ranibizumab, except that it includes one, two, three, four, five, six, seven, eight, nine, ten, eleven, twelve, thirteen, fourteen, or fifteen amino acid substitutions, and/or includes a variable heavy chain domain that is or includes the sequence of variable heavy chain of ranibizumab, except that it includes one, two, three, four, five, six, seven, eight, nine, ten, eleven, twelve, thirteen, fourteen, or fifteen amino acid substitutions. In some embodiments the first antigen-binding domain includes the three CDRs in the light chain variable domain of ranibizumab, and/or the three CDRs in the heavy chain variable domain of ranibizumab.

### Vectors

The compositions provided herein include at least two (e.g., two, three, four, five, or six) nucleic acid vectors, where: each of the at least two different vectors includes a coding sequence that encodes a different portion of a target protein (e.g., a supporting cell target protein or a hair cell target protein), each of the encoded portions being at least 30 amino acids (e.g., about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 250 amino acids, about 30 amino acids to about 200 amino acids, about 30 amino acids to about 150 amino acids, about 30 amino acids to about 100 amino acids, about 30 amino acids to about 50 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 1600 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 800 amino acids, about 600 amino acids to about 750 amino acids, about 600 amino acids to about 700 amino acids, about 600 amino acids to about 650 amino acids, about 650 amino acids to about 800 amino acids, about 650 amino acids to about 750 amino acids, about 650 amino acids to about 700 amino acids, about 700 amino acids to about 800 amino acids, about 700 amino acids to about 750 amino acids, or about 750 amino acids to about 800 amino acids), where the amino acid sequence of each of the encoded portions may optionally partially overlap with the amino acid sequence of a different one of the encoded portions; no single vector of the at least two different vectors encodes the active target protein (e.g., an active supporting cell target protein (e.g., a full-length supporting cell target protein (e.g., a full-length wildtype supporting cell target protein)) or an active hair cell target protein (e.g., a full-length hair cell target protein (e.g., a full-length wildtype hair cell target protein)); and, when introduced into a primate cell, the at least two different vectors undergo homologous recombination with each other, where the recombined nucleic acid encodes an active target protein (e.g., an active supporting cell target protein (e.g., a full-length supporting cell target protein) or an active hair cell target protein (e.g., a full-length hair cell target protein). In some embodiments, one of the nucleic acid vectors can include a coding sequence that encodes a portion of a target protein (e.g., a supporting cell target protein or a hair cell target protein), where the encoded portion is, e.g., about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 250 amino acids, about 30 amino acids to about 200 amino acids, about 30 amino acids to about 150 amino acids, about 30 amino acids to about 100 amino acids, about 30 amino acids to about 50 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 1600 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 800 amino acids, about 600 amino acids to about 750 amino acids, about 600 amino acids to about 700 amino acids, about 600 amino acids to about 650 amino acids, about 650 amino acids to about 800 amino acids, about 650 amino acids to about 750 amino acids, about 650 amino acids to about 700 amino acids, about 700 amino acids to about 800 amino acids, about 700 amino acids to about 750 amino acids, or about 750 amino acids to about 800 amino acids in length.

In some embodiments of these compositions, at least one of the coding sequences includes a nucleotide sequence spanning two neighboring exons of target genomic DNA (e.g., supporting cell target genomic DNA or hair cell target genomic DNA), and lacks the intronic sequence that naturally occurs between the two neighboring exons.

In some embodiments, the amino acid sequence of none of the encoded portions overlaps even in part with the amino acid sequence of a different one of the encoded portions. In some embodiments, the amino acid sequence of one or more of the encoded portions partially overlaps with the amino acid sequence of a different one of the encoded portions. In some embodiments, the amino acid sequence of each of the encoded portions partially overlaps with the amino acid sequence of a different one of the encoded portions.

In some embodiments, the overlapping amino acid sequence is between about 30 amino acid residues to about 800 amino acids (e.g., or any of the subranges of this range described herein) in length.

In some examples, the vectors include two different vectors, each of which includes a different segment of an intron, where the intron includes the nucleotide sequence of an intron that is present in a target genomic DNA (e.g., a supporting cell target genomic DNA (e.g., SLC26A4 genomic DNA or a GJB2 genomic DNA) (e.g., any of the exemplary introns in SEQ ID NO: 3 described herein) or a hair cell target genomic DNA (e.g., an OTOF genomic DNA, a TRIOBP genomic DNA, a PJVK genomic DNA, a NDP genomic DNA, a CLRN1 genomic DNA, a TMC1 genomic DNA, a VEGF genomic DNA, a HSPA1A genomic DNA, or a STRC genomic DNA (e.g., any of the exemplary introns in SEQ ID NO: 19, SEQ ID NO: 22, SEQ ID NO: 33, SEQ ID NO: 37, SEQ ID NO: 47, SEQ ID NO: 50, SEQ ID NO: 61, SEQ ID NO: 71 or SEQ ID NO: 80 described herein), and where the two different segments overlap in sequence by at least 100 nucleotides (e.g., about 100 nucleotides to about 3,000 nucleotides, about 100 nucleotides to about 2,500 nucleotides, about 100 nucleotides to about 2,000 nucleotides, about 100 nucleotides to about 1,500 nucleotides, about 100 nucleotides to about 1,000 nucleotides, about 100 nucleotides to about 800 nucleotides, about 100 nucleotides to about 600 nucleotides, about 100 nucleotides to about 400 nucleotides, about 100 nucleotides to about 200 nucleotides, about 200 nucleotides to about 3,000 nucleotides, about 200 nucleotides to about 2,500 nucleotides, about 200 nucleotides to about 2,000 nucleotides, about 200 nucleotides to about 1,500 nucleotides, about 200 nucleotides to about 1,000 nucleotides, about 200 nucleotides to about 800 nucleotides, about 200 nucleotides to about 600 nucleotides, about 200 nucleotides to about 400 nucleotides, about 400 nucleotides to about 3,000 nucleotides, about 400 nucleotides to about 2,500 nucleotides, about 400 nucleotides to about 2,000 nucleotides, about 400 nucleotides to about 1,500 nucleotides, about 400 nucleotides to about 1,000 nucleotides, about 400 nucleotides to about 800 nucleotides, about 400 nucleotides to about 600 nucleotides, about 600 nucleotides to about 3,000 nucleotides, about 600 nucleotides to about 2,500 nucleotides, about 600 nucleotides to about 2,000 nucleotides, about 600 nucleotides to about 1,500 nucleotides, about 600 nucleotides to about 1,000 nucleotides, about 600 nucleotides to about 800 nucleotides, about 800 nucleotides to about 3,000 nucleotides, about 800 nucleotides to about 2,500 nucleotides, about 800 nucleotides to about 2,000 nucleotides, about 800 nucleotides to about 1,500 nucleotides, about 800 nucleotides to about 1,000 nucleotides, about 1,000 nucleotides to about 3,000 nucleotides, about 1,000 nucleotides to about 2,500 nucleotides, about 1,000 nucleotides to about 2,000 nucleotides, about 1,000 nucleotides to about 1,500 nucleotides, about 1,500 nucleotides to about 3,000 nucleotides, about 1,500 nucleotides to about 2,500 nucleotides, about 1,500 nucleotides to about 2,000 nucleotides, about 2,000 nucleotides to about 3,000 nucleotides, about 2,000 nucleotides to about 2,500 nucleotides, or about 2,500 nucleotides to about 3,000 nucleotides), in length.

The overlapping nucleotide sequence in any two of the different vectors can include part or all of one or more exons of a target gene (e.g., a supporting cell target gene (e.g., a SLC26A4 gene or a GJB2 gene) (e.g., any one or more of the exemplary exons in SEQ ID NO: 3 described herein) or a hair cell target gene (e.g., an OTOF gene, a TRIOBP gene, a PJVK gene, a NDP gene, a CLRN1 gene, a TMC1 gene, or a STRC gene) (e.g., any one or more of the exemplary exons in SEQ ID NO: 19, SEQ ID NO: 22, SEQ ID NO: 33, SEQ ID NO: 37, SEQ ID NO: 47, SEQ ID NO: 50, SEQ ID NO: 63, SEQ ID NO: 71 or SEQ ID NO: 80 described herein).

In some embodiments, the number of different vectors in the composition is two, three, four, or five. In compositions where the number of different vectors in the composition is two, the first of the two different vectors can include a coding sequence that encodes an N-terminal portion of the target protein (e.g., an N-terminal portion of the supporting cell target protein or an N-terminal portion of the hair cell target protein). In some examples, the N-terminal portion of the target gene (e.g., the N-terminal portion of the supporting cell target gene or the N-terminal portion of the hair cell target gene) is between about 30 amino acids to about 800 amino acids (e.g., between about 30 amino acids to about 790 amino acids, between about 30 amino acids to about 780 amino acids, between about 30 amino acids to about 770 amino acids, between about 30 amino acids to about 760 amino acids, between about 30 amino acids to about 750 amino acids, between about 30 amino acids to about 740 amino acids, between about 30 amino acids to about 730 amino acids, between about 30 amino acids to about 720 amino acids, between about 30 amino acids to about 710 amino acids, between about 30 amino acids to about 700 amino acids, between about 30 amino acids to about 690 amino acids, between about 30 amino acids to about 680 amino acids, between 30 amino acids to about 670 amino acids, between about 30 amino acids to about 660 amino acids, between about 30 amino acids to about 650 amino acids, between about 30 amino acids to about 640 amino acids, between about 30 amino acids to about 630 amino acids, between about 30 amino acids to about 620 amino acids, between about 30 amino acids to about 610 amino acids, between about 30 amino acids to about 600 amino acids, between about 30 amino acids to about 590 amino acids, between about 30 amino acids to about 580 amino acids, between about 30 amino acids to about 570 amino acids, between about 30 amino acids to about 560 amino acids, between about 30 amino acids to about 550 amino acids, between about 30 amino acids to about 540 amino acids, between about 30 amino acids to about 530 amino acids, between about 30 amino acids to about 520 amino acids, between about 30 amino acids to about 510 amino acids, between about 30 amino acids to about 500 amino acids, between about 30 amino acids to about 490 amino acids, between about 30 amino acids to about 480 amino acids, between about 30 amino acids to about 470 amino acids, between about 30 amino acids to about 460 amino acids, between about 30 amino acids to about 450 amino acids, between about 30 amino acids to about 440 amino acids, between about 30 amino acids to about 430 amino acids, between about 30 amino acids to about 420 amino acids, between about 30 amino acids to about 410 amino acids, between about 30 amino acids to about 400 amino acids, between about 30 amino acids to about 390 amino acids, between about 30 amino acids to about 380 amino acids, between about 30 amino acids to about 370 amino acids, between about 30 amino acids to about 360 amino acids, between about 30 amino acids to about 350 amino acids, between about 30 amino acids to about 340 amino acids, between about 30 amino acids to about 330 amino acids, between about 30 amino acids to about 320 amino acids, between about 30 amino acids to about 310 amino acids, between about 30 amino acids to about 300 amino acids, between about 30 amino acids to about 290 amino acids, between about 30 amino acids to about 280 amino acids, between about 30 amino acids to about 270 amino acids, between about 30 amino acids to about 260 amino acids, between about 30 amino acids to about 250 amino acids, between about 30 amino acids to about 240 amino acids, between about 30 amino acids to about 230 amino acids, between about 30 amino acids to about 220 amino acids, between about 30 amino acids to about 210 amino acids, between about 30 amino acids to about 200 amino acids, between about 30 amino acids to about 190 amino acids, between about 30 amino acids to about 180 amino acids, between about 30 amino acids to about 170 amino acids, between about 30 amino acids to about 160 amino acids, between about 30 amino acids to about 150 amino acids, between about 30 amino acids to about 140 amino acids, between about 30 amino acids to about 130 amino acids, between about 30 amino acids to about 120 amino acids, between about 30 amino acids to about 110 amino acids, between about 30 amino acids to about 100 amino acids, between about 30 amino acids to about 90 amino acids, between about 30 amino acids to about 80 amino acids, between about 30 amino acids to about 70 amino acids, between about 30 amino acids to about 60 amino acids, between about 30 amino acids to about 50 amino acids, between about 30 amino acids to about 40 amino acids, between about 50 amino acids to about 800 amino acids, between about 50 amino acids to about 790 amino acids, between about 50 amino acids to about 780 amino acids, between about 50 amino acids to about 770 amino acids, between about 50 amino acids to about 760 amino acids, between about 50 amino acids to about 750 amino acids, between about 50 amino acids to about 740 amino acids, between about 50 amino acids to about 730 amino acids, between about 50 amino acids to about 720 amino acids, between about 50 amino acids to about 710 amino acids, between about 50 amino acids to about 700 amino acids, between about 50 amino acids to about 690 amino acids, between about 50 amino acids to about 680 amino acids, between 30 amino acids to about 670 amino acids, between about 50 amino acids to about 660 amino acids, between about 50 amino acids to about 650 amino acids, between about 50 amino acids to about 640 amino acids, between about 30 amino acids to about 630 amino acids, between about 50 amino acids to about 620 amino acids, between about 50 amino acids to about 610 amino acids, between about 50 amino acids to about 600 amino acids, between about 50 amino acids to about 590 amino acids, between about 50 amino acids to about 580 amino acids, between about 50 amino acids to about 570 amino acids, between about 50 amino acids to about 560 amino acids, between about 50 amino acids to about 550 amino acids, between about 50 amino acids to about 540 amino acids, between about 50 amino acids to about 530 amino acids, between about 50 amino acids to about 520 amino acids, between about 50 amino acids to about 510 amino acids, between about 50 amino acids to about 500 amino acids, between about 50 amino acids to about 490 amino acids, between about 50 amino acids to about 480 amino acids, between about 50 amino acids to about 470 amino acids, between about 50 amino acids to about 460 amino acids, between about 50 amino acids to about 450 amino acids, between about 50 amino acids to about 440 amino acids, between about 50 amino acids to about 430 amino acids, between about 50 amino acids to about 420 amino acids, between about 50 amino acids to about 410 amino acids, between about 50 amino acids to about 400 amino acids, between about 50 amino acids to about 390 amino acids, between about 50 amino acids to about 380 amino acids, between about 50 amino acids to about 370 amino acids, between about 50 amino acids to about 360 amino acids, between about 50 amino acids to about 350 amino acids, between about 50 amino acids to about 340 amino acids, between about 50 amino acids to about 330 amino acids, between about 50 amino acids to about 320 amino acids, between about 50 amino acids to about 310 amino acids, between about 50 amino acids to about 300 amino acids, between about 50 amino acids to about 290 amino acids, between about 50 amino acids to about 280 amino acids, between about 50 amino acids to about 270 amino acids, between about 50 amino acids to about 260 amino acids, between about 50 amino acids to about 250 amino acids, between about 50 amino acids to about 240 amino acids, between about 50 amino acids to about 230 amino acids, between about 50 amino acids to about 220 amino acids, between about 50 amino acids to about 210 amino acids, between about 50 amino acids to about 200 amino acids, between about 50 amino acids to about 190 amino acids, between about 50 amino acids to about 180 amino acids, between about 50 amino acids to about 170 amino acids, between about 50 amino acids to about 160 amino acids, between about 50 amino acids to about 150 amino acids, between about 50 amino acids to about 140 amino acids, between about 50 amino acids to about 130 amino acids, between about 50 amino acids to about 120 amino acids, between about 50 amino acids to about 110 amino acids, between about 50 amino acids to about 100 amino acids, between about 50 amino acids to about 90 amino acids, between about 50 amino acids to about 80 amino acids, between about 50 amino acids to about 70 amino acids, between about 50 amino acids to about 60 amino acids, between about 80 amino acids to about 800 amino acids, between about 80 amino acids to about 790 amino acids, between about 80 amino acids to about 780 amino acids, between about 80 amino acids to about 770 amino acids, between about 80 amino acids to about 760 amino acids, between about 80 amino acids to about 750 amino acids, between about 80 amino acids to about 740 amino acids, between about 80 amino acids to about 730 amino acids, between about 80 amino acids to about 720 amino acids, between about 80 amino acids to about 710 amino acids, between about 80 amino acids to about 700 amino acids, between about 80 amino acids to about 690 amino acids, between about 80 amino acids to about 680 amino acids, between 30 amino acids to about 670 amino acids, between about 80 amino acids to about 660 amino acids, between about 80 amino acids to about 650 amino acids, between about 80 amino acids to about 640 amino acids, between about 30 amino acids to about 630 amino acids, between about 80 amino acids to about 620 amino acids, between about 80 amino acids to about 610 amino acids, between about 80 amino acids to about 600 amino acids, between about 80 amino acids to about 590 amino acids, between about 80 amino acids to about 580 amino acids, between about 80 amino acids to about 570 amino acids, between about 80 amino acids to about 560 amino acids, between about 80 amino acids to about 550 amino acids, between about 80 amino acids to about 540 amino acids, between about 80 amino acids to about 530 amino acids, between about 80 amino acids to about 520 amino acids, between about 80 amino acids to about 510 amino acids, between about 80 amino acids to about 500 amino acids, between about 80 amino acids to about 490 amino acids, between about 80 amino acids to about 480 amino acids, between about 80 amino acids to about 470 amino acids, between about 80 amino acids to about 460 amino acids, between about 80 amino acids to about 450 amino acids, between about 80 amino acids to about 440 amino acids, between about 80 amino acids to about 430 amino acids, between about 80 amino acids to about 420 amino acids, between about 80 amino acids to about 410 amino acids, between about 80 amino acids to about 400 amino acids, between about 80 amino acids to about 390 amino acids, between about 80 amino acids to about 380 amino acids, between about 80 amino acids to about 370 amino acids, between about 80 amino acids to about 360 amino acids, between about 80 amino acids to about 350 amino acids, between about 80 amino acids to about 340 amino acids, between about 80 amino acids to about 330 amino acids, between about 80 amino acids to about 320 amino acids, between about 80 amino acids to about 310 amino acids, between about 80 amino acids to about 300 amino acids, between about 80 amino acids to about 290 amino acids, between about 80 amino acids to about 280 amino acids, between about 80 amino acids to about 270 amino acids, between about 80 amino acids to about 260 amino acids, between about 80 amino acids to about 250 amino acids, between about 80 amino acids to about 240 amino acids, between about 80 amino acids to about 230 amino acids, between about 80 amino acids to about 220 amino acids, between about 80 amino acids to about 210 amino acids, between about 80 amino acids to about 200 amino acids, between about 80 amino acids to about 190 amino acids, between about 80 amino acids to about 180 amino acids, between about 80 amino acids to about 170 amino acids, between about 80 amino acids to about 160 amino acids, between about 80 amino acids to about 150 amino acids, between about 80 amino acids to about 140 amino acids, between about 80 amino acids to about 130 amino acids, between about 80 amino acids to about 120 amino acids, between about 80 amino acids to about 110 amino acids, between about 80 amino acids to about 100 amino acids, between about 100 amino acids to about 800 amino acids, between about 100 amino acids to about 790 amino acids, between about 100 amino acids to about 780 amino acids, between about 100 amino acids to about 770 amino acids, between about 100 amino acids to about 760 amino acids, between about 100 amino acids to about 750 amino acids, between about 100 amino acids to about 740 amino acids, between about 100 amino acids to about 730 amino acids, between about 100 amino acids to about 720 amino acids, between about 100 amino acids to about 710 amino acids, between about 100 amino acids to about 700 amino acids, between about 100 amino acids to about 690 amino acids, between about 100 amino acids to about 680 amino acids, between 30 amino acids to about 670 amino acids, between about 100 amino acids to about 660 amino acids, between about 100 amino acids to about 650 amino acids, between about 100 amino acids to about 640 amino acids, between about 30 amino acids to about 630 amino acids, between about 100 amino acids to about 620 amino acids, between about 100 amino acids to about 610 amino acids, between about 100 amino acids to about 600 amino acids, between about 100 amino acids to about 590 amino acids, between about 100 amino acids to about 580 amino acids, between about 100 amino acids to about 570 amino acids, between about 100 amino acids to about 560 amino acids, between about 100 amino acids to about 550 amino acids, between about 100 amino acids to about 540 amino acids, between about 100 amino acids to about 530 amino acids, between about 100 amino acids to about 520 amino acids, between about 100 amino acids to about 510 amino acids, between about 100 amino acids to about 500 amino acids, between about 100 amino acids to about 490 amino acids, between about 100 amino acids to about 480 amino acids, between about 100 amino acids to about 470 amino acids, between about 100 amino acids to about 460 amino acids, between about 100 amino acids to about 450 amino acids, between about 100 amino acids to about 440 amino acids, between about 100 amino acids to about 430 amino acids, between about 100 amino acids to about 420 amino acids, between about 100 amino acids to about 410 amino acids, between about 100 amino acids to about 400 amino acids, between about 100 amino acids to about 390 amino acids, between about 100 amino acids to about 380 amino acids, between about 100 amino acids to about 370 amino acids, between about 100 amino acids to about 360 amino acids, between about 100 amino acids to about 350 amino acids, between about 100 amino acids to about 340 amino acids, between about 100 amino acids to about 330 amino acids, between about 100 amino acids to about 320 amino acids, between about 100 amino acids to about 310 amino acids, between about 100 amino acids to about 300 amino acids, between about 100 amino acids to about 290 amino acids, between about 100 amino acids to about 280 amino acids, between about 100 amino acids to about 270 amino acids, between about 100 amino acids to about 260 amino acids, between about 100 amino acids to about 250 amino acids, between about 100 amino acids to about 240 amino acids, between about 100 amino acids to about 230 amino acids, between about 100 amino acids to about 220 amino acids, between about 100 amino acids to about 210 amino acids, between about 100 amino acids to about 200 amino acids, between about 100 amino acids to about 190 amino acids, between about 100 amino acids to about 180 amino acids, between about 100 amino acids to about 170 amino acids, between about 100 amino acids to about 160 amino acids, between about 100 amino acids to about 150 amino acids, between about 100 amino acids to about 140 amino acids, between about 100 amino acids to about 130 amino acids, between about 100 amino acids to about 120 amino acids, between about 100 amino acids to about 110 amino acids, between about 120 amino acids to about 800 amino acids, between about 120 amino acids to about 790 amino acids, between about 120 amino acids to about 780 amino acids, between about 120 amino acids to about 770 amino acids, between about 120 amino acids to about 760 amino acids, between about 120 amino acids to about 750 amino acids, between about 120 amino acids to about 740 amino acids, between about 120 amino acids to about 730 amino acids, between about 120 amino acids to about 720 amino acids, between about 120 amino acids to about 710 amino acids, between about 120 amino acids to about 700 amino acids, between about 120 amino acids to about 690 amino acids, between about 120 amino acids to about 680 amino acids, between 30 amino acids to about 670 amino acids, between about 120 amino acids to about 660 amino acids, between about 120 amino acids to about 650 amino acids, between about 120 amino acids to about 640 amino acids, between about 30 amino acids to about 630 amino acids, between about 120 amino acids to about 620 amino acids, between about 120 amino acids to about 610 amino acids, between about 120 amino acids to about 600 amino acids, between about 120 amino acids to about 590 amino acids, between about 120 amino acids to about 580 amino acids, between about 120 amino acids to about 570 amino acids, between about 120 amino acids to about 560 amino acids, between about 120 amino acids to about 550 amino acids, between about 120 amino acids to about 540 amino acids, between about 120 amino acids to about 530 amino acids, between about 120 amino acids to about 520 amino acids, between about 120 amino acids to about 510 amino acids, between about 120 amino acids to about 500 amino acids, between about 120 amino acids to about 490 amino acids, between about 120 amino acids to about 480 amino acids, between about 120 amino acids to about 470 amino acids, between about 120 amino acids to about 460 amino acids, between about 120 amino acids to about 450 amino acids, between about 120 amino acids to about 440 amino acids, between about 120 amino acids to about 430 amino acids, between about 120 amino acids to about 420 amino acids, between about 120 amino acids to about 410 amino acids, between about 120 amino acids to about 400 amino acids, between about 120 amino acids to about 390 amino acids, between about 120 amino acids to about 380 amino acids, between about 120 amino acids to about 370 amino acids, between about 120 amino acids to about 360 amino acids, between about 120 amino acids to about 350 amino acids, between about 120 amino acids to about 340 amino acids, between about 120 amino acids to about 330 amino acids, between about 120 amino acids to about 320 amino acids, between about 120 amino acids to about 310 amino acids, between about 120 amino acids to about 300 amino acids, between about 120 amino acids to about 290 amino acids, between about 120 amino acids to about 280 amino acids, between about 120 amino acids to about 270 amino acids, between about 120 amino acids to about 260 amino acids, between about 120 amino acids to about 250 amino acids, between about 120 amino acids to about 240 amino acids, between about 120 amino acids to about 230 amino acids, between about 120 amino acids to about 220 amino acids, between about 120 amino acids to about 210 amino acids, between about 120 amino acids to about 200 amino acids, between about 120 amino acids to about 190 amino acids, between about 120 amino acids to about 180 amino acids, between about 120 amino acids to about 170 amino acids, between about 120 amino acids to about 160 amino acids, between about 120 amino acids to about 150 amino acids, between about 120 amino acids to about 140 amino acids, between about 120 amino acids to about 130 amino acids, between about 150 amino acids to about 800 amino acids, between about 150 amino acids to about 790 amino acids, between about 150 amino acids to about 780 amino acids, between about 150 amino acids to about 770 amino acids, between about 150 amino acids to about 760 amino acids, between about 150 amino acids to about 750 amino acids, between about 150 amino acids to about 740 amino acids, between about 150 amino acids to about 730 amino acids, between about 150 amino acids to about 720 amino acids, between about 150 amino acids to about 710 amino acids, between about 150 amino acids to about 700 amino acids, between about 150 amino acids to about 690 amino acids, between about 150 amino acids to about 680 amino acids, between 30 amino acids to about 670 amino acids, between about 150 amino acids to about 660 amino acids, between about 150 amino acids to about 650 amino acids, between about 150 amino acids to about 640 amino acids, between about 30 amino acids to about 630 amino acids, between about 150 amino acids to about 620 amino acids, between about 150 amino acids to about 610 amino acids, between about 150 amino acids to about 600 amino acids, between about 150 amino acids to about 590 amino acids, between about 150 amino acids to about 580 amino acids, between about 150 amino acids to about 570 amino acids, between about 150 amino acids to about 560 amino acids, between about 150 amino acids to about 550 amino acids, between about 150 amino acids to about 540 amino acids, between about 150 amino acids to about 530 amino acids, between about 150 amino acids to about 520 amino acids, between about 150 amino acids to about 510 amino acids, between about 150 amino acids to about 500 amino acids, between about 150 amino acids to about 490 amino acids, between about 150 amino acids to about 480 amino acids, between about 150 amino acids to about 470 amino acids, between about 150 amino acids to about 460 amino acids, between about 150 amino acids to about 450 amino acids, between about 150 amino acids to about 440 amino acids, between about 150 amino acids to about 430 amino acids, between about 150 amino acids to about 420 amino acids, between about 150 amino acids to about 410 amino acids, between about 150 amino acids to about 400 amino acids, between about 150 amino acids to about 390 amino acids, between about 150 amino acids to about 380 amino acids, between about 150 amino acids to about 370 amino acids, between about 150 amino acids to about 360 amino acids, between about 150 amino acids to about 350 amino acids, between about 150 amino acids to about 340 amino acids, between about 150 amino acids to about 330 amino acids, between about 150 amino acids to about 320 amino acids, between about 150 amino acids to about 310 amino acids, between about 150 amino acids to about 300 amino acids, between about 150 amino acids to about 290 amino acids, between about 150 amino acids to about 280 amino acids, between about 150 amino acids to about 270 amino acids, between about 150 amino acids to about 260 amino acids, between about 150 amino acids to about 250 amino acids, between about 150 amino acids to about 240 amino acids, between about 150 amino acids to about 230 amino acids, between about 150 amino acids to about 220 amino acids, between about 150 amino acids to about 210 amino acids, between about 150 amino acids to about 200 amino acids, between about 150 amino acids to about 190 amino acids, between about 150 amino acids to about 180 amino acids, between about 150 amino acids to about 170 amino acids, between about 150 amino acids to about 160 amino acids, between about 180 amino acids to about 800 amino acids, between about 180 amino acids to about 790 amino acids, between about 180 amino acids to about 780 amino acids, between about 180 amino acids to about 770 amino acids, between about 180 amino acids to about 760 amino acids, between about 180 amino acids to about 750 amino acids, between about 180 amino acids to about 740 amino acids, between about 180 amino acids to about 730 amino acids, between about 180 amino acids to about 720 amino acids, between about 180 amino acids to about 710 amino acids, between about 180 amino acids to about 700 amino acids, between about 180 amino acids to about 690 amino acids, between about 180 amino acids to about 680 amino acids, between 30 amino acids to about 670 amino acids, between about 180 amino acids to about 660 amino acids, between about 180 amino acids to about 650 amino acids, between about 180 amino acids to about 640 amino acids, between about 30 amino acids to about 630 amino acids, between about 180 amino acids to about 620 amino acids, between about 180 amino acids to about 610 amino acids, between about 180 amino acids to about 600 amino acids, between about 180 amino acids to about 590 amino acids, between about 180 amino acids to about 580 amino acids, between about 180 amino acids to about 570 amino acids, between about 180 amino acids to about 560 amino acids, between about 180 amino acids to about 550 amino acids, between about 180 amino acids to about 540 amino acids, between about 180 amino acids to about 530 amino acids, between about 180 amino acids to about 520 amino acids, between about 180 amino acids to about 510 amino acids, between about 180 amino acids to about 500 amino acids, between about 180 amino acids to about 490 amino acids, between about 180 amino acids to about 480 amino acids, between about 180 amino acids to about 470 amino acids, between about 180 amino acids to about 460 amino acids, between about 180 amino acids to about 450 amino acids, between about 180 amino acids to about 440 amino acids, between about 180 amino acids to about 430 amino acids, between about 180 amino acids to about 420 amino acids, between about 180 amino acids to about 410 amino acids, between about 180 amino acids to about 400 amino acids, between about 180 amino acids to about 390 amino acids, between about 180 amino acids to about 380 amino acids, between about 180 amino acids to about 370 amino acids, between about 180 amino acids to about 360 amino acids, between about 180 amino acids to about 350 amino acids, between about 180 amino acids to about 340 amino acids, between about 180 amino acids to about 330 amino acids, between about 180 amino acids to about 320 amino acids, between about 180 amino acids to about 310 amino acids, between about 180 amino acids to about 300 amino acids, between about 180 amino acids to about 290 amino acids, between about 180 amino acids to about 280 amino acids, between about 180 amino acids to about 270 amino acids, between about 180 amino acids to about 260 amino acids, between about 180 amino acids to about 250 amino acids, between about 180 amino acids to about 240 amino acids, between about 180 amino acids to about 230 amino acids, between about 180 amino acids to about 220 amino acids, between about 180 amino acids to about 210 amino acids, between about 180 amino acids to about 200 amino acids, between about 180 amino acids to about 190 amino acids, between about 200 amino acids to about 800 amino acids, between about 200 amino acids to about 790 amino acids, between about 200 amino acids to about 780 amino acids, between about 200 amino acids to about 770 amino acids, between about 200 amino acids to about 760 amino acids, between about 200 amino acids to about 750 amino acids, between about 200 amino acids to about 740 amino acids, between about 200 amino acids to about 730 amino acids, between about 200 amino acids to about 720 amino acids, between about 200 amino acids to about 710 amino acids, between about 200 amino acids to about 700 amino acids, between about 200 amino acids to about 690 amino acids, between about 200 amino acids to about 680 amino acids, between 30 amino acids to about 670 amino acids, between about 200 amino acids to about 660 amino acids, between about 200 amino acids to about 650 amino acids, between about 200 amino acids to about 640 amino acids, between about 30 amino acids to about 630 amino acids, between about 200 amino acids to about 620 amino acids, between about 200 amino acids to about 610 amino acids, between about 200 amino acids to about 600 amino acids, between about 200 amino acids to about 590 amino acids, between about 200 amino acids to about 580 amino acids, between about 200 amino acids to about 570 amino acids, between about 200 amino acids to about 560 amino acids, between about 200 amino acids to about 550 amino acids, between about 200 amino acids to about 540 amino acids, between about 200 amino acids to about 530 amino acids, between about 200 amino acids to about 520 amino acids, between about 200 amino acids to about 510 amino acids, between about 200 amino acids to about 500 amino acids, between about 200 amino acids to about 490 amino acids, between about 200 amino acids to about 480 amino acids, between about 200 amino acids to about 470 amino acids, between about 200 amino acids to about 460 amino acids, between about 200 amino acids to about 450 amino acids, between about 200 amino acids to about 440 amino acids, between about 200 amino acids to about 430 amino acids, between about 200 amino acids to about 420 amino acids, between about 200 amino acids to about 410 amino acids, between about 200 amino acids to about 400 amino acids, between about 200 amino acids to about 390 amino acids, between about 200 amino acids to about 380 amino acids, between about 200 amino acids to about 370 amino acids, between about 200 amino acids to about 360 amino acids, between about 200 amino acids to about 350 amino acids, between about 200 amino acids to about 340 amino acids, between about 200 amino acids to about 330 amino acids, between about 200 amino acids to about 320 amino acids, between about 200 amino acids to about 310 amino acids, between about 200 amino acids to about 300 amino acids, between about 200 amino acids to about 290 amino acids, between about 200 amino acids to about 280 amino acids, between about 200 amino acids to about 270 amino acids, between about 200 amino acids to about 260 amino acids, between about 200 amino acids to about 250 amino acids, between about 200 amino acids to about 240 amino acids, between about 200 amino acids to about 230 amino acids, between about 200 amino acids to about 220 amino acids, between about 200 amino acids to about 210 amino acids, between about 250 amino acids to about 800 amino acids, between about 250 amino acids to about 790 amino acids, between about 250 amino acids to about 780 amino acids, between about 250 amino acids to about 770 amino acids, between about 250 amino acids to about 760 amino acids, between about 250 amino acids to about 750 amino acids, between about 250 amino acids to about 740 amino acids, between about 250 amino acids to about 730 amino acids, between about 250 amino acids to about 720 amino acids, between about 250 amino acids to about 710 amino acids, between about 250 amino acids to about 700 amino acids, between about 250 amino acids to about 690 amino acids, between about 250 amino acids to about 680 amino acids, between 30 amino acids to about 670 amino acids, between about 250 amino acids to about 660 amino acids, between about 250 amino acids to about 650 amino acids, between about 250 amino acids to about 640 amino acids, between about 30 amino acids to about 630 amino acids, between about 250 amino acids to about 620 amino acids, between about 250 amino acids to about 610 amino acids, between about 250 amino acids to about 600 amino acids, between about 250 amino acids to about 590 amino acids, between about 250 amino acids to about 580 amino acids, between about 250 amino acids to about 570 amino acids, between about 250 amino acids to about 560 amino acids, between about 250 amino acids to about 550 amino acids, between about 250 amino acids to about 540 amino acids, between about 250 amino acids to about 530 amino acids, between about 250 amino acids to about 520 amino acids, between about 250 amino acids to about 510 amino acids, between about 250 amino acids to about 500 amino acids, between about 250 amino acids to about 490 amino acids, between about 250 amino acids to about 480 amino acids, between about 250 amino acids to about 470 amino acids, between about 250 amino acids to about 460 amino acids, between about 250 amino acids to about 450 amino acids, between about 250 amino acids to about 440 amino acids, between about 250 amino acids to about 430 amino acids, between about 250 amino acids to about 420 amino acids, between about 250 amino acids to about 410 amino acids, between about 250 amino acids to about 400 amino acids, between about 250 amino acids to about 390 amino acids, between about 250 amino acids to about 380 amino acids, between about 250 amino acids to about 370 amino acids, between about 250 amino acids to about 360 amino acids, between about 250 amino acids to about 350 amino acids, between about 250 amino acids to about 340 amino acids, between about 250 amino acids to about 330 amino acids, between about 250 amino acids to about 320 amino acids, between about 250 amino acids to about 310 amino acids, between about 250 amino acids to about 300 amino acids, between about 250 amino acids to about 290 amino acids, between about 250 amino acids to about 280 amino acids, between about 250 amino acids to about 270 amino acids, between about 250 amino acids to about 260 amino acids, between about 280 amino acids to about 800 amino acids, between about 280 amino acids to about 790 amino acids, between about 280 amino acids to about 780 amino acids, between about 280 amino acids to about 770 amino acids, between about 280 amino acids to about 760 amino acids, between about 280 amino acids to about 750 amino acids, between about 280 amino acids to about 740 amino acids, between about 280 amino acids to about 730 amino acids, between about 280 amino acids to about 720 amino acids, between about 280 amino acids to about 710 amino acids, between about 280 amino acids to about 700 amino acids, between about 280 amino acids to about 690 amino acids, between about 280 amino acids to about 680 amino acids, between 30 amino acids to about 670 amino acids, between about 280 amino acids to about 660 amino acids, between about 280 amino acids to about 650 amino acids, between about 280 amino acids to about 640 amino acids, between about 30 amino acids to about 630 amino acids, between about 280 amino acids to about 620 amino acids, between about 280 amino acids to about 610 amino acids, between about 280 amino acids to about 600 amino acids, between about 280 amino acids to about 590 amino acids, between about 280 amino acids to about 580 amino acids, between about 280 amino acids to about 570 amino acids, between about 280 amino acids to about 560 amino acids, between about 280 amino acids to about 550 amino acids, between about 280 amino acids to about 540 amino acids, between about 280 amino acids to about 530 amino acids, between about 280 amino acids to about 520 amino acids, between about 280 amino acids to about 510 amino acids, between about 280 amino acids to about 500 amino acids, between about 280 amino acids to about 490 amino acids, between about 280 amino acids to about 480 amino acids, between about 280 amino acids to about 470 amino acids, between about 280 amino acids to about 460 amino acids, between about 280 amino acids to about 450 amino acids, between about 280 amino acids to about 440 amino acids, between about 280 amino acids to about 430 amino acids, between about 280 amino acids to about 420 amino acids, between about 280 amino acids to about 410 amino acids, between about 280 amino acids to about 400 amino acids, between about 280 amino acids to about 390 amino acids, between about 280 amino acids to about 380 amino acids, between about 280 amino acids to about 370 amino acids, between about 280 amino acids to about 360 amino acids, between about 280 amino acids to about 350 amino acids, between about 280 amino acids to about 340 amino acids, between about 280 amino acids to about 330 amino acids, between about 280 amino acids to about 320 amino acids, between about 280 amino acids to about 310 amino acids, between about 280 amino acids to about 300 amino acids, between about 300 amino acids to about 800 amino acids, between about 300 amino acids to about 790 amino acids, between about 300 amino acids to about 780 amino acids, between about 300 amino acids to about 770 amino acids, between about 300 amino acids to about 760 amino acids, between about 300 amino acids to about 750 amino acids, between about 300 amino acids to about 740 amino acids, between about 300 amino acids to about 730 amino acids, between about 300 amino acids to about 720 amino acids, between about 300 amino acids to about 710 amino acids, between about 300 amino acids to about 700 amino acids, between about 300 amino acids to about 690 amino acids, between about 300 amino acids to about 680 amino acids, between 30 amino acids to about 670 amino acids, between about 300 amino acids to about 660 amino acids, between about 300 amino acids to about 650 amino acids, between about 300 amino acids to about 640 amino acids, between about 30 amino acids to about 630 amino acids, between about 300 amino acids to about 620 amino acids, between about 300 amino acids to about 610 amino acids, between about 300 amino acids to about 600 amino acids, between about 300 amino acids to about 590 amino acids, between about 300 amino acids to about 580 amino acids, between about 300 amino acids to about 570 amino acids, between about 300 amino acids to about 560 amino acids, between about 300 amino acids to about 550 amino acids, between about 300 amino acids to about 540 amino acids, between about 300 amino acids to about 530 amino acids, between about 300 amino acids to about 520 amino acids, between about 300 amino acids to about 510 amino acids, between about 300 amino acids to about 500 amino acids, between about 300 amino acids to about 490 amino acids, between about 300 amino acids to about 480 amino acids, between about 300 amino acids to about 470 amino acids, between about 300 amino acids to about 460 amino acids, between about 300 amino acids to about 450 amino acids, between about 300 amino acids to about 440 amino acids, between about 300 amino acids to about 430 amino acids, between about 300 amino acids to about 420 amino acids, between about 300 amino acids to about 410 amino acids, between about 300 amino acids to about 400 amino acids, between about 300 amino acids to about 390 amino acids, between about 300 amino acids to about 380 amino acids, between about 300 amino acids to about 370 amino acids, between about 300 amino acids to about 360 amino acids, between about 300 amino acids to about 350 amino acids, between about 300 amino acids to about 340 amino acids, between about 300 amino acids to about 330 amino acids, between about 300 amino acids to about 320 amino acids, between about 300 amino acids to about 310 amino acids, between about 350 amino acids to about 800 amino acids, between about 350 amino acids to about 790 amino acids, between about 350 amino acids to about 780 amino acids, between about 350 amino acids to about 770 amino acids, between about 350 amino acids to about 760 amino acids, between about 350 amino acids to about 750 amino acids, between about 350 amino acids to about 740 amino acids, between about 350 amino acids to about 730 amino acids, between about 350 amino acids to about 720 amino acids, between about 350 amino acids to about 710 amino acids, between about 350 amino acids to about 700 amino acids, between about 350 amino acids to about 690 amino acids, between about 350 amino acids to about 680 amino acids, between 30 amino acids to about 670 amino acids, between about 350 amino acids to about 660 amino acids, between about 350 amino acids to about 650 amino acids, between about 350 amino acids to about 640 amino acids, between about 30 amino acids to about 630 amino acids, between about 350 amino acids to about 620 amino acids, between about 350 amino acids to about 610 amino acids, between about 350 amino acids to about 600 amino acids, between about 350 amino acids to about 590 amino acids, between about 350 amino acids to about 580 amino acids, between about 350 amino acids to about 570 amino acids, between about 350 amino acids to about 560 amino acids, between about 350 amino acids to about 550 amino acids, between about 350 amino acids to about 540 amino acids, between about 350 amino acids to about 530 amino acids, between about 350 amino acids to about 520 amino acids, between about 350 amino acids to about 510 amino acids, between about 350 amino acids to about 500 amino acids, between about 350 amino acids to about 490 amino acids, between about 350 amino acids to about 480 amino acids, between about 350 amino acids to about 470 amino acids, between about 350 amino acids to about 460 amino acids, between about 350 amino acids to about 450 amino acids, between about 350 amino acids to about 440 amino acids, between about 350 amino acids to about 430 amino acids, between about 350 amino acids to about 420 amino acids, between about 350 amino acids to about 410 amino acids, between about 350 amino acids to about 400 amino acids, between about 350 amino acids to about 390 amino acids, between about 350 amino acids to about 380 amino acids, between about 350 amino acids to about 370 amino acids, between about 350 amino acids to about 360 amino acids, between about 380 amino acids to about 800 amino acids, between about 380 amino acids to about 790 amino acids, between about 380 amino acids to about 780 amino acids, between about 380 amino acids to about 770 amino acids, between about 380 amino acids to about 760 amino acids, between about 380 amino acids to about 750 amino acids, between about 380 amino acids to about 740 amino acids, between about 380 amino acids to about 730 amino acids, between about 380 amino acids to about 720 amino acids, between about 380 amino acids to about 710 amino acids, between about 380 amino acids to about 700 amino acids, between about 380 amino acids to about 690 amino acids, between about 380 amino acids to about 680 amino acids, between 30 amino acids to about 670 amino acids, between about 380 amino acids to about 660 amino acids, between about 380 amino acids to about 650 amino acids, between about 380 amino acids to about 640 amino acids, between about 30 amino acids to about 630 amino acids, between about 380 amino acids to about 620 amino acids, between about 380 amino acids to about 610 amino acids, between about 380 amino acids to about 600 amino acids, between about 380 amino acids to about 590 amino acids, between about 380 amino acids to about 580 amino acids, between about 380 amino acids to about 570 amino acids, between about 380 amino acids to about 560 amino acids, between about 380 amino acids to about 550 amino acids, between about 380 amino acids to about 540 amino acids, between about 380 amino acids to about 530 amino acids, between about 380 amino acids to about 520 amino acids, between about 380 amino acids to about 510 amino acids, between about 380 amino acids to about 500 amino acids, between about 380 amino acids to about 490 amino acids, between about 380 amino acids to about 480 amino acids, between about 380 amino acids to about 470 amino acids, between about 380 amino acids to about 460 amino acids, between about 380 amino acids to about 450 amino acids, between about 380 amino acids to about 440 amino acids, between about 380 amino acids to about 430 amino acids, between about 380 amino acids to about 420 amino acids, between about 380 amino acids to about 410 amino acids, between about 380 amino acids to about 400 amino acids, between about 380 amino acids to about 390 amino acids, between about 400 amino acids to about 800 amino acids, between about 400 amino acids to about 790 amino acids, between about 400 amino acids to about 780 amino acids, between about 400 amino acids to about 770 amino acids, between about 400 amino acids to about 760 amino acids, between about 400 amino acids to about 750 amino acids, between about 400 amino acids to about 740 amino acids, between about 400 amino acids to about 730 amino acids, between about 400 amino acids to about 720 amino acids, between about 400 amino acids to about 710 amino acids, between about 400 amino acids to about 700 amino acids, between about 400 amino acids to about 690 amino acids, between about 400 amino acids to about 680 amino acids, between 30 amino acids to about 670 amino acids, between about 400 amino acids to about 660 amino acids, between about 400 amino acids to about 650 amino acids, between about 400 amino acids to about 640 amino acids, between about 30 amino acids to about 630 amino acids, between about 400 amino acids to about 620 amino acids, between about 400 amino acids to about 610 amino acids, between about 400 amino acids to about 600 amino acids, between about 400 amino acids to about 590 amino acids, between about 400 amino acids to about 580 amino acids, between about 400 amino acids to about 570 amino acids, between about 400 amino acids to about 560 amino acids, between about 400 amino acids to about 550 amino acids, between about 400 amino acids to about 540 amino acids, between about 400 amino acids to about 530 amino acids, between about 400 amino acids to about 520 amino acids, between about 400 amino acids to about 510 amino acids, between about 400 amino acids to about 500 amino acids, between about 400 amino acids to about 490 amino acids, between about 400 amino acids to about 480 amino acids, between about 400 amino acids to about 470 amino acids, between about 400 amino acids to about 460 amino acids, between about 400 amino acids to about 450 amino acids, between about 400 amino acids to about 440 amino acids, between about 400 amino acids to about 430 amino acids, between about 400 amino acids to about 420 amino acids, between about 400 amino acids to about 410 amino acids, between about 450 amino acids to about 800 amino acids, between about 450 amino acids to about 790 amino acids, between about 450 amino acids to about 780 amino acids, between about 450 amino acids to about 770 amino acids, between about 450 amino acids to about 760 amino acids, between about 450 amino acids to about 750 amino acids, between about 450 amino acids to about 740 amino acids, between about 450 amino acids to about 730 amino acids, between about 450 amino acids to about 720 amino acids, between about 450 amino acids to about 710 amino acids, between about 450 amino acids to about 700 amino acids, between about 450 amino acids to about 690 amino acids, between about 450 amino acids to about 680 amino acids, between 30 amino acids to about 670 amino acids, between about 450 amino acids to about 660 amino acids, between about 450 amino acids to about 650 amino acids, between about 450 amino acids to about 640 amino acids, between about 30 amino acids to about 630 amino acids, between about 450 amino acids to about 620 amino acids, between about 450 amino acids to about 610 amino acids, between about 450 amino acids to about 600 amino acids, between about 450 amino acids to about 590 amino acids, between about 450 amino acids to about 580 amino acids, between about 450 amino acids to about 570 amino acids, between about 450 amino acids to about 560 amino acids, between about 450 amino acids to about 550 amino acids, between about 450 amino acids to about 540 amino acids, between about 450 amino acids to about 530 amino acids, between about 450 amino acids to about 520 amino acids, between about 450 amino acids to about 510 amino acids, between about 450 amino acids to about 500 amino acids, between about 450 amino acids to about 490 amino acids, between about 450 amino acids to about 480 amino acids, between about 450 amino acids to about 470 amino acids, between about 450 amino acids to about 460 amino acids, between about 480 amino acids to about 800 amino acids, between about 480 amino acids to about 790 amino acids, between about 480 amino acids to about 780 amino acids, between about 480 amino acids to about 770 amino acids, between about 480 amino acids to about 760 amino acids, between about 480 amino acids to about 750 amino acids, between about 480 amino acids to about 740 amino acids, between about 480 amino acids to about 730 amino acids, between about 480 amino acids to about 720 amino acids, between about 480 amino acids to about 710 amino acids, between about 480 amino acids to about 700 amino acids, between about 480 amino acids to about 690 amino acids, between about 480 amino acids to about 680 amino acids, between 30 amino acids to about 670 amino acids, between about 480 amino acids to about 660 amino acids, between about 480 amino acids to about 650 amino acids, between about 480 amino acids to about 640 amino acids, between about 30 amino acids to about 630 amino acids, between about 480 amino acids to about 620 amino acids, between about 480 amino acids to about 610 amino acids, between about 480 amino acids to about 600 amino acids, between about 480 amino acids to about 590 amino acids, between about 480 amino acids to about 580 amino acids, between about 480 amino acids to about 570 amino acids, between about 480 amino acids to about 560 amino acids, between about 480 amino acids to about 550 amino acids, between about 480 amino acids to about 540 amino acids, between about 480 amino acids to about 530 amino acids, between about 480 amino acids to about 520 amino acids, between about 480 amino acids to about 510 amino acids, between about 480 amino acids to about 500 amino acids, between about 480 amino acids to about 490 amino acids, between about 50 amino acids to about 800 amino acids, between about 50 amino acids to about 790 amino acids, between about 50 amino acids to about 780 amino acids, between about 50 amino acids to about 770 amino acids, between about 50 amino acids to about 760 amino acids, between about 50 amino acids to about 750 amino acids, between about 50 amino acids to about 740 amino acids, between about 50 amino acids to about 730 amino acids, between about 50 amino acids to about 720 amino acids, between about 50 amino acids to about 710 amino acids, between about 50 amino acids to about 700 amino acids, between about 50 amino acids to about 690 amino acids, between about 50 amino acids to about 680 amino acids, between 30 amino acids to about 670 amino acids, between about 50 amino acids to about 660 amino acids, between about 50 amino acids to about 650 amino acids, between about 50 amino acids to about 640 amino acids, between about 30 amino acids to about 630 amino acids, between about 50 amino acids to about 620 amino acids, between about 50 amino acids to about 610 amino acids, between about 50 amino acids to about 600 amino acids, between about 50 amino acids to about 590 amino acids, between about 50 amino acids to about 580 amino acids, between about 50 amino acids to about 570 amino acids, between about 50 amino acids to about 560 amino acids, between about 50 amino acids to about 550 amino acids, between about 50 amino acids to about 540 amino acids, between about 50 amino acids to about 530 amino acids, between about 50 amino acids to about 520 amino acids, between about 50 amino acids to about 510 amino acids, between about 550 amino acids to about 800 amino acids, between about 550 amino acids to about 790 amino acids, between about 550 amino acids to about 780 amino acids, between about 550 amino acids to about 770 amino acids, between about 550 amino acids to about 760 amino acids, between about 550 amino acids to about 750 amino acids, between about 550 amino acids to about 740 amino acids, between about 550 amino acids to about 730 amino acids, between about 550 amino acids to about 720 amino acids, between about 550 amino acids to about 710 amino acids, between about 550 amino acids to about 700 amino acids, between about 550 amino acids to about 690 amino acids, between about 550 amino acids to about 680 amino acids, between 30 amino acids to about 670 amino acids, between about 550 amino acids to about 660 amino acids, between about 550 amino acids to about 650 amino acids, between about 550 amino acids to about 640 amino acids, between about 30 amino acids to about 630 amino acids, between about 550 amino acids to about 620 amino acids, between about 550 amino acids to about 610 amino acids, between about 550 amino acids to about 600 amino acids, between about 550 amino acids to about 590 amino acids, between about 550 amino acids to about 580 amino acids, between about 550 amino acids to about 570 amino acids, between about 550 amino acids to about 560 amino acids, between about 580 amino acids to about 800 amino acids, between about 580 amino acids to about 790 amino acids, between about 580 amino acids to about 780 amino acids, between about 580 amino acids to about 770 amino acids, between about 580 amino acids to about 760 amino acids, between about 580 amino acids to about 750 amino acids, between about 580 amino acids to about 740 amino acids, between about 580 amino acids to about 730 amino acids, between about 580 amino acids to about 720 amino acids, between about 580 amino acids to about 710 amino acids, between about 580 amino acids to about 700 amino acids, between about 580 amino acids to about 690 amino acids, between about 580 amino acids to about 680 amino acids, between 30 amino acids to about 670 amino acids, between about 580 amino acids to about 660 amino acids, between about 580 amino acids to about 650 amino acids, between about 580 amino acids to about 640 amino acids, between about 30 amino acids to about 630 amino acids, between about 580 amino acids to about 620 amino acids, between about 580 amino acids to about 610 amino acids, between about 580 amino acids to about 600 amino acids, between about 580 amino acids to about 590 amino acids, between about 600 amino acids to about 800 amino acids, between about 600 amino acids to about 790 amino acids, between about 600 amino acids to about 780 amino acids, between about 600 amino acids to about 770 amino acids, between about 600 amino acids to about 760 amino acids, between about 600 amino acids to about 750 amino acids, between about 600 amino acids to about 740 amino acids, between about 600 amino acids to about 730 amino acids, between about 600 amino acids to about 720 amino acids, between about 600 amino acids to about 710 amino acids, between about 600 amino acids to about 700 amino acids, between about 600 amino acids to about 690 amino acids, between about 600 amino acids to about 680 amino acids, between 30 amino acids to about 670 amino acids, between about 600 amino acids to about 660 amino acids, between about 600 amino acids to about 650 amino acids, between about 600 amino acids to about 640 amino acids, between about 30 amino acids to about 630 amino acids, between about 600 amino acids to about 620 amino acids, between about 600 amino acids to about 610 amino acids, between about 650 amino acids to about 800 amino acids, between about 650 amino acids to about 790 amino acids, between about 650 amino acids to about 780 amino acids, between about 650 amino acids to about 770 amino acids, between about 650 amino acids to about 760 amino acids, between about 650 amino acids to about 750 amino acids, between about 650 amino acids to about 740 amino acids, between about 650 amino acids to about 730 amino acids, between about 650 amino acids to about 720 amino acids, between about 650 amino acids to about 710 amino acids, between about 650 amino acids to about 700 amino acids, between about 650 amino acids to about 690 amino acids, between about 650 amino acids to about 680 amino acids, between 30 amino acids to about 670 amino acids, between about 650 amino acids to about 660 amino acids, between about 680 amino acids to about 800 amino acids, between about 680 amino acids to about 790 amino acids, between about 680 amino acids to about 780 amino acids, between about 680 amino acids to about 770 amino acids, between about 680 amino acids to about 760 amino acids, between about 680 amino acids to about 750 amino acids, between about 680 amino acids to about 740 amino acids, between about 680 amino acids to about 730 amino acids, between about 680 amino acids to about 720 amino acids, between about 680 amino acids to about 710 amino acids, between about 680 amino acids to about 700 amino acids, between about 680 amino acids to about 690 amino acids, between about 700 amino acids to about 800 amino acids, between about 700 amino acids to about 790 amino acids, between about 700 amino acids to about 780 amino acids, between about 700 amino acids to about 770 amino acids, between about 700 amino acids to about 760 amino acids, between about 700 amino acids to about 750 amino acids, between about 700 amino acids to about 740 amino acids, between about 700 amino acids to about 730 amino acids, between about 700 amino acids to about 720 amino acids, between about 700 amino acids to about 710 amino acids, between about 750 amino acids to about 800 amino acids, between about 750 amino acids to about 790 amino acids, between about 750 amino acids to about 780 amino acids, between about 750 amino acids to about 770 amino acids, between about 750 amino acids to about 760 amino acids, or between about 780 amino acids to about 800 amino acids) in length. In some examples, the first vector further includes one or both of a promoter (e.g., any of the promoters described herein or known in the art) and a Kozak sequence (e.g., any of the exemplary Kozak sequences described herein or known in the art). In some examples, the first vector includes a promoter that is an inducible promoter, a constitutive promoter, or a tissue-specific promoter. In some examples, the second of the two different vectors includes a coding sequence that encodes a C-terminal portion of the target protein (e.g., a C-terminal portion of the supporting cell target protein or a C-terminal portion of the hair cell target protein). In some examples, the C-terminal portion of the target protein (e.g., the C-terminal portion of the supporting cell target protein or the C-terminal portion of the hair cell target protein) is between about 30 amino acids to about 800 amino acids (e.g., between about 30 amino acids to about 790 amino acids, between about 30 amino acids to about 780 amino acids, between about 30 amino acids to about 770 amino acids, between about 30 amino acids to about 760 amino acids, between about 30 amino acids to about 750 amino acids, between about 30 amino acids to about 740 amino acids, between about 30 amino acids to about 730 amino acids, between about 30 amino acids to about 720 amino acids, between about 30 amino acids to about 710 amino acids, between about 30 amino acids to about 700 amino acids, between about 30 amino acids to about 690 amino acids, between about 30 amino acids to about 680 amino acids, between 30 amino acids to about 670 amino acids, between about 30 amino acids to about 660 amino acids, between about 30 amino acids to about 650 amino acids, between about 30 amino acids to about 640 amino acids, between about 30 amino acids to about 630 amino acids, between about 30 amino acids to about 620 amino acids, between about 30 amino acids to about 610 amino acids, between about 30 amino acids to about 600 amino acids, between about 30 amino acids to about 590 amino acids, between about 30 amino acids to about 580 amino acids, between about 30 amino acids to about 570 amino acids, between about 30 amino acids to about 560 amino acids, between about 30 amino acids to about 550 amino acids, between about 30 amino acids to about 540 amino acids, between about 30 amino acids to about 530 amino acids, between about 30 amino acids to about 520 amino acids, between about 30 amino acids to about 510 amino acids, between about 30 amino acids to about 500 amino acids, between about 30 amino acids to about 490 amino acids, between about 30 amino acids to about 480 amino acids, between about 30 amino acids to about 470 amino acids, between about 30 amino acids to about 460 amino acids, between about 30 amino acids to about 450 amino acids, between about 30 amino acids to about 440 amino acids, between about 30 amino acids to about 430 amino acids, between about 30 amino acids to about 420 amino acids, between about 30 amino acids to about 410 amino acids, between about 30 amino acids to about 400 amino acids, between about 30 amino acids to about 390 amino acids, between about 30 amino acids to about 380 amino acids, between about 30 amino acids to about 370 amino acids, between about 30 amino acids to about 360 amino acids, between about 30 amino acids to about 350 amino acids, between about 30 amino acids to about 340 amino acids, between about 30 amino acids to about 330 amino acids, between about 30 amino acids to about 320 amino acids, between about 30 amino acids to about 310 amino acids, between about 30 amino acids to about 300 amino acids, between about 30 amino acids to about 290 amino acids, between about 30 amino acids to about 280 amino acids, between about 30 amino acids to about 270 amino acids, between about 30 amino acids to about 260 amino acids, between about 30 amino acids to about 250 amino acids, between about 30 amino acids to about 240 amino acids, between about 30 amino acids to about 230 amino acids, between about 30 amino acids to about 220 amino acids, between about 30 amino acids to about 210 amino acids, between about 30 amino acids to about 200 amino acids, between about 30 amino acids to about 190 amino acids, between about 30 amino acids to about 180 amino acids, between about 30 amino acids to about 170 amino acids, between about 30 amino acids to about 160 amino acids, between about 30 amino acids to about 150 amino acids, between about 30 amino acids to about 140 amino acids, between about 30 amino acids to about 130 amino acids, between about 30 amino acids to about 120 amino acids, between about 30 amino acids to about 110 amino acids, between about 30 amino acids to about 100 amino acids, between about 30 amino acids to about 90 amino acids, between about 30 amino acids to about 80 amino acids, between about 30 amino acids to about 70 amino acids, between about 30 amino acids to about 60 amino acids, between about 30 amino acids to about 50 amino acids, between about 30 amino acids to about 40 amino acids, between about 50 amino acids to about 800 amino acids, between about 50 amino acids to about 790 amino acids, between about 50 amino acids to about 780 amino acids, between about 50 amino acids to about 770 amino acids, between about 50 amino acids to about 760 amino acids, between about 50 amino acids to about 750 amino acids, between about 50 amino acids to about 740 amino acids, between about 50 amino acids to about 730 amino acids, between about 50 amino acids to about 720 amino acids, between about 50 amino acids to about 710 amino acids, between about 50 amino acids to about 700 amino acids, between about 50 amino acids to about 690 amino acids, between about 50 amino acids to about 680 amino acids, between 30 amino acids to about 670 amino acids, between about 50 amino acids to about 660 amino acids, between about 50 amino acids to about 650 amino acids, between about 50 amino acids to about 640 amino acids, between about 30 amino acids to about 630 amino acids, between about 50 amino acids to about 620 amino acids, between about 50 amino acids to about 610 amino acids, between about 50 amino acids to about 600 amino acids, between about 50 amino acids to about 590 amino acids, between about 50 amino acids to about 580 amino acids, between about 50 amino acids to about 570 amino acids, between about 50 amino acids to about 560 amino acids, between about 50 amino acids to about 550 amino acids, between about 50 amino acids to about 540 amino acids, between about 50 amino acids to about 530 amino acids, between about 50 amino acids to about 520 amino acids, between about 50 amino acids to about 510 amino acids, between about 50 amino acids to about 500 amino acids, between about 50 amino acids to about 490 amino acids, between about 50 amino acids to about 480 amino acids, between about 50 amino acids to about 470 amino acids, between about 50 amino acids to about 460 amino acids, between about 50 amino acids to about 450 amino acids, between about 50 amino acids to about 440 amino acids, between about 50 amino acids to about 430 amino acids, between about 50 amino acids to about 420 amino acids, between about 50 amino acids to about 410 amino acids, between about 50 amino acids to about 400 amino acids, between about 50 amino acids to about 390 amino acids, between about 50 amino acids to about 380 amino acids, between about 50 amino acids to about 370 amino acids, between about 50 amino acids to about 360 amino acids, between about 50 amino acids to about 350 amino acids, between about 50 amino acids to about 340 amino acids, between about 50 amino acids to about 330 amino acids, between about 50 amino acids to about 320 amino acids, between about 50 amino acids to about 310 amino acids, between about 50 amino acids to about 300 amino acids, between about 50 amino acids to about 290 amino acids, between about 50 amino acids to about 280 amino acids, between about 50 amino acids to about 270 amino acids, between about 50 amino acids to about 260 amino acids, between about 50 amino acids to about 250 amino acids, between about 50 amino acids to about 240 amino acids, between about 50 amino acids to about 230 amino acids, between about 50 amino acids to about 220 amino acids, between about 50 amino acids to about 210 amino acids, between about 50 amino acids to about 200 amino acids, between about 50 amino acids to about 190 amino acids, between about 50 amino acids to about 180 amino acids, between about 50 amino acids to about 170 amino acids, between about 50 amino acids to about 160 amino acids, between about 50 amino acids to about 150 amino acids, between about 50 amino acids to about 140 amino acids, between about 50 amino acids to about 130 amino acids, between about 50 amino acids to about 120 amino acids, between about 50 amino acids to about 110 amino acids, between about 50 amino acids to about 100 amino acids, between about 50 amino acids to about 90 amino acids, between about 50 amino acids to about 80 amino acids, between about 50 amino acids to about 70 amino acids, between about 50 amino acids to about 60 amino acids, between about 80 amino acids to about 800 amino acids, between about 80 amino acids to about 790 amino acids, between about 80 amino acids to about 780 amino acids, between about 80 amino acids to about 770 amino acids, between about 80 amino acids to about 760 amino acids, between about 80 amino acids to about 750 amino acids, between about 80 amino acids to about 740 amino acids, between about 80 amino acids to about 730 amino acids, between about 80 amino acids to about 720 amino acids, between about 80 amino acids to about 710 amino acids, between about 80 amino acids to about 700 amino acids, between about 80 amino acids to about 690 amino acids, between about 80 amino acids to about 680 amino acids, between 30 amino acids to about 670 amino acids, between about 80 amino acids to about 660 amino acids, between about 80 amino acids to about 650 amino acids, between about 80 amino acids to about 640 amino acids, between about 30 amino acids to about 630 amino acids, between about 80 amino acids to about 620 amino acids, between about 80 amino acids to about 610 amino acids, between about 80 amino acids to about 600 amino acids, between about 80 amino acids to about 590 amino acids, between about 80 amino acids to about 580 amino acids, between about 80 amino acids to about 570 amino acids, between about 80 amino acids to about 560 amino acids, between about 80 amino acids to about 550 amino acids, between about 80 amino acids to about 540 amino acids, between about 80 amino acids to about 530 amino acids, between about 80 amino acids to about 520 amino acids, between about 80 amino acids to about 510 amino acids, between about 80 amino acids to about 500 amino acids, between about 80 amino acids to about 490 amino acids, between about 80 amino acids to about 480 amino acids, between about 80 amino acids to about 470 amino acids, between about 80 amino acids to about 460 amino acids, between about 80 amino acids to about 450 amino acids, between about 80 amino acids to about 440 amino acids, between about 80 amino acids to about 430 amino acids, between about 80 amino acids to about 420 amino acids, between about 80 amino acids to about 410 amino acids, between about 80 amino acids to about 400 amino acids, between about 80 amino acids to about 390 amino acids, between about 80 amino acids to about 380 amino acids, between about 80 amino acids to about 370 amino acids, between about 80 amino acids to about 360 amino acids, between about 80 amino acids to about 350 amino acids, between about 80 amino acids to about 340 amino acids, between about 80 amino acids to about 330 amino acids, between about 80 amino acids to about 320 amino acids, between about 80 amino acids to about 310 amino acids, between about 80 amino acids to about 300 amino acids, between about 80 amino acids to about 290 amino acids, between about 80 amino acids to about 280 amino acids, between about 80 amino acids to about 270 amino acids, between about 80 amino acids to about 260 amino acids, between about 80 amino acids to about 250 amino acids, between about 80 amino acids to about 240 amino acids, between about 80 amino acids to about 230 amino acids, between about 80 amino acids to about 220 amino acids, between about 80 amino acids to about 210 amino acids, between about 80 amino acids to about 200 amino acids, between about 80 amino acids to about 190 amino acids, between about 80 amino acids to about 180 amino acids, between about 80 amino acids to about 170 amino acids, between about 80 amino acids to about 160 amino acids, between about 80 amino acids to about 150 amino acids, between about 80 amino acids to about 140 amino acids, between about 80 amino acids to about 130 amino acids, between about 80 amino acids to about 120 amino acids, between about 80 amino acids to about 110 amino acids, between about 80 amino acids to about 100 amino acids, between about 100 amino acids to about 800 amino acids, between about 100 amino acids to about 790 amino acids, between about 100 amino acids to about 780 amino acids, between about 100 amino acids to about 770 amino acids, between about 100 amino acids to about 760 amino acids, between about 100 amino acids to about 750 amino acids, between about 100 amino acids to about 740 amino acids, between about 100 amino acids to about 730 amino acids, between about 100 amino acids to about 720 amino acids, between about 100 amino acids to about 710 amino acids, between about 100 amino acids to about 700 amino acids, between about 100 amino acids to about 690 amino acids, between about 100 amino acids to about 680 amino acids, between 30 amino acids to about 670 amino acids, between about 100 amino acids to about 660 amino acids, between about 100 amino acids to about 650 amino acids, between about 100 amino acids to about 640 amino acids, between about 30 amino acids to about 630 amino acids, between about 100 amino acids to about 620 amino acids, between about 100 amino acids to about 610 amino acids, between about 100 amino acids to about 600 amino acids, between about 100 amino acids to about 590 amino acids, between about 100 amino acids to about 580 amino acids, between about 100 amino acids to about 570 amino acids, between about 100 amino acids to about 560 amino acids, between about 100 amino acids to about 550 amino acids, between about 100 amino acids to about 540 amino acids, between about 100 amino acids to about 530 amino acids, between about 100 amino acids to about 520 amino acids, between about 100 amino acids to about 510 amino acids, between about 100 amino acids to about 500 amino acids, between about 100 amino acids to about 490 amino acids, between about 100 amino acids to about 480 amino acids, between about 100 amino acids to about 470 amino acids, between about 100 amino acids to about 460 amino acids, between about 100 amino acids to about 450 amino acids, between about 100 amino acids to about 440 amino acids, between about 100 amino acids to about 430 amino acids, between about 100 amino acids to about 420 amino acids, between about 100 amino acids to about 410 amino acids, between about 100 amino acids to about 400 amino acids, between about 100 amino acids to about 390 amino acids, between about 100 amino acids to about 380 amino acids, between about 100 amino acids to about 370 amino acids, between about 100 amino acids to about 360 amino acids, between about 100 amino acids to about 350 amino acids, between about 100 amino acids to about 340 amino acids, between about 100 amino acids to about 330 amino acids, between about 100 amino acids to about 320 amino acids, between about 100 amino acids to about 310 amino acids, between about 100 amino acids to about 300 amino acids, between about 100 amino acids to about 290 amino acids, between about 100 amino acids to about 280 amino acids, between about 100 amino acids to about 270 amino acids, between about 100 amino acids to about 260 amino acids, between about 100 amino acids to about 250 amino acids, between about 100 amino acids to about 240 amino acids, between about 100 amino acids to about 230 amino acids, between about 100 amino acids to about 220 amino acids, between about 100 amino acids to about 210 amino acids, between about 100 amino acids to about 200 amino acids, between about 100 amino acids to about 190 amino acids, between about 100 amino acids to about 180 amino acids, between about 100 amino acids to about 170 amino acids, between about 100 amino acids to about 160 amino acids, between about 100 amino acids to about 150 amino acids, between about 100 amino acids to about 140 amino acids, between about 100 amino acids to about 130 amino acids, between about 100 amino acids to about 120 amino acids, between about 100 amino acids to about 110 amino acids, between about 120 amino acids to about 800 amino acids, between about 120 amino acids to about 790 amino acids, between about 120 amino acids to about 780 amino acids, between about 120 amino acids to about 770 amino acids, between about 120 amino acids to about 760 amino acids, between about 120 amino acids to about 750 amino acids, between about 120 amino acids to about 740 amino acids, between about 120 amino acids to about 730 amino acids, between about 120 amino acids to about 720 amino acids, between about 120 amino acids to about 710 amino acids, between about 120 amino acids to about 700 amino acids, between about 120 amino acids to about 690 amino acids, between about 120 amino acids to about 680 amino acids, between 30 amino acids to about 670 amino acids, between about 120 amino acids to about 660 amino acids, between about 120 amino acids to about 650 amino acids, between about 120 amino acids to about 640 amino acids, between about 30 amino acids to about 630 amino acids, between about 120 amino acids to about 620 amino acids, between about 120 amino acids to about 610 amino acids, between about 120 amino acids to about 600 amino acids, between about 120 amino acids to about 590 amino acids, between about 120 amino acids to about 580 amino acids, between about 120 amino acids to about 570 amino acids, between about 120 amino acids to about 560 amino acids, between about 120 amino acids to about 550 amino acids, between about 120 amino acids to about 540 amino acids, between about 120 amino acids to about 530 amino acids, between about 120 amino acids to about 520 amino acids, between about 120 amino acids to about 510 amino acids, between about 120 amino acids to about 500 amino acids, between about 120 amino acids to about 490 amino acids, between about 120 amino acids to about 480 amino acids, between about 120 amino acids to about 470 amino acids, between about 120 amino acids to about 460 amino acids, between about 120 amino acids to about 450 amino acids, between about 120 amino acids to about 440 amino acids, between about 120 amino acids to about 430 amino acids, between about 120 amino acids to about 420 amino acids, between about 120 amino acids to about 410 amino acids, between about 120 amino acids to about 400 amino acids, between about 120 amino acids to about 390 amino acids, between about 120 amino acids to about 380 amino acids, between about 120 amino acids to about 370 amino acids, between about 120 amino acids to about 360 amino acids, between about 120 amino acids to about 350 amino acids, between about 120 amino acids to about 340 amino acids, between about 120 amino acids to about 330 amino acids, between about 120 amino acids to about 320 amino acids, between about 120 amino acids to about 310 amino acids, between about 120 amino acids to about 300 amino acids, between about 120 amino acids to about 290 amino acids, between about 120 amino acids to about 280 amino acids, between about 120 amino acids to about 270 amino acids, between about 120 amino acids to about 260 amino acids, between about 120 amino acids to about 250 amino acids, between about 120 amino acids to about 240 amino acids, between about 120 amino acids to about 230 amino acids, between about 120 amino acids to about 220 amino acids, between about 120 amino acids to about 210 amino acids, between about 120 amino acids to about 200 amino acids, between about 120 amino acids to about 190 amino acids, between about 120 amino acids to about 180 amino acids, between about 120 amino acids to about 170 amino acids, between about 120 amino acids to about 160 amino acids, between about 120 amino acids to about 150 amino acids, between about 120 amino acids to about 140 amino acids, between about 120 amino acids to about 130 amino acids, between about 150 amino acids to about 800 amino acids, between about 150 amino acids to about 790 amino acids, between about 150 amino acids to about 780 amino acids, between about 150 amino acids to about 770 amino acids, between about 150 amino acids to about 760 amino acids, between about 150 amino acids to about 750 amino acids, between about 150 amino acids to about 740 amino acids, between about 150 amino acids to about 730 amino acids, between about 150 amino acids to about 720 amino acids, between about 150 amino acids to about 710 amino acids, between about 150 amino acids to about 700 amino acids, between about 150 amino acids to about 690 amino acids, between about 150 amino acids to about 680 amino acids, between 30 amino acids to about 670 amino acids, between about 150 amino acids to about 660 amino acids, between about 150 amino acids to about 650 amino acids, between about 150 amino acids to about 640 amino acids, between about 30 amino acids to about 630 amino acids, between about 150 amino acids to about 620 amino acids, between about 150 amino acids to about 610 amino acids, between about 150 amino acids to about 600 amino acids, between about 150 amino acids to about 590 amino acids, between about 150 amino acids to about 580 amino acids, between about 150 amino acids to about 570 amino acids, between about 150 amino acids to about 560 amino acids, between about 150 amino acids to about 550 amino acids, between about 150 amino acids to about 540 amino acids, between about 150 amino acids to about 530 amino acids, between about 150 amino acids to about 520 amino acids, between about 150 amino acids to about 510 amino acids, between about 150 amino acids to about 500 amino acids, between about 150 amino acids to about 490 amino acids, between about 150 amino acids to about 480 amino acids, between about 150 amino acids to about 470 amino acids, between about 150 amino acids to about 460 amino acids, between about 150 amino acids to about 450 amino acids, between about 150 amino acids to about 440 amino acids, between about 150 amino acids to about 430 amino acids, between about 150 amino acids to about 420 amino acids, between about 150 amino acids to about 410 amino acids, between about 150 amino acids to about 400 amino acids, between about 150 amino acids to about 390 amino acids, between about 150 amino acids to about 380 amino acids, between about 150 amino acids to about 370 amino acids, between about 150 amino acids to about 360 amino acids, between about 150 amino acids to about 350 amino acids, between about 150 amino acids to about 340 amino acids, between about 150 amino acids to about 330 amino acids, between about 150 amino acids to about 320 amino acids, between about 150 amino acids to about 310 amino acids, between about 150 amino acids to about 300 amino acids, between about 150 amino acids to about 290 amino acids, between about 150 amino acids to about 280 amino acids, between about 150 amino acids to about 270 amino acids, between about 150 amino acids to about 260 amino acids, between about 150 amino acids to about 250 amino acids, between about 150 amino acids to about 240 amino acids, between about 150 amino acids to about 230 amino acids, between about 150 amino acids to about 220 amino acids, between about 150 amino acids to about 210 amino acids, between about 150 amino acids to about 200 amino acids, between about 150 amino acids to about 190 amino acids, between about 150 amino acids to about 180 amino acids, between about 150 amino acids to about 170 amino acids, between about 150 amino acids to about 160 amino acids, between about 180 amino acids to about 800 amino acids, between about 180 amino acids to about 790 amino acids, between about 180 amino acids to about 780 amino acids, between about 180 amino acids to about 770 amino acids, between about 180 amino acids to about 760 amino acids, between about 180 amino acids to about 750 amino acids, between about 180 amino acids to about 740 amino acids, between about 180 amino acids to about 730 amino acids, between about 180 amino acids to about 720 amino acids, between about 180 amino acids to about 710 amino acids, between about 180 amino acids to about 700 amino acids, between about 180 amino acids to about 690 amino acids, between about 180 amino acids to about 680 amino acids, between 30 amino acids to about 670 amino acids, between about 180 amino acids to about 660 amino acids, between about 180 amino acids to about 650 amino acids, between about 180 amino acids to about 640 amino acids, between about 30 amino acids to about 630 amino acids, between about 180 amino acids to about 620 amino acids, between about 180 amino acids to about 610 amino acids, between about 180 amino acids to about 600 amino acids, between about 180 amino acids to about 590 amino acids, between about 180 amino acids to about 580 amino acids, between about 180 amino acids to about 570 amino acids, between about 180 amino acids to about 560 amino acids, between about 180 amino acids to about 550 amino acids, between about 180 amino acids to about 540 amino acids, between about 180 amino acids to about 530 amino acids, between about 180 amino acids to about 520 amino acids, between about 180 amino acids to about 510 amino acids, between about 180 amino acids to about 500 amino acids, between about 180 amino acids to about 490 amino acids, between about 180 amino acids to about 480 amino acids, between about 180 amino acids to about 470 amino acids, between about 180 amino acids to about 460 amino acids, between about 180 amino acids to about 450 amino acids, between about 180 amino acids to about 440 amino acids, between about 180 amino acids to about 430 amino acids, between about 180 amino acids to about 420 amino acids, between about 180 amino acids to about 410 amino acids, between about 180 amino acids to about 400 amino acids, between about 180 amino acids to about 390 amino acids, between about 180 amino acids to about 380 amino acids, between about 180 amino acids to about 370 amino acids, between about 180 amino acids to about 360 amino acids, between about 180 amino acids to about 350 amino acids, between about 180 amino acids to about 340 amino acids, between about 180 amino acids to about 330 amino acids, between about 180 amino acids to about 320 amino acids, between about 180 amino acids to about 310 amino acids, between about 180 amino acids to about 300 amino acids, between about 180 amino acids to about 290 amino acids, between about 180 amino acids to about 280 amino acids, between about 180 amino acids to about 270 amino acids, between about 180 amino acids to about 260 amino acids, between about 180 amino acids to about 250 amino acids, between about 180 amino acids to about 240 amino acids, between about 180 amino acids to about 230 amino acids, between about 180 amino acids to about 220 amino acids, between about 180 amino acids to about 210 amino acids, between about 180 amino acids to about 200 amino acids, between about 180 amino acids to about 190 amino acids, between about 200 amino acids to about 800 amino acids, between about 200 amino acids to about 790 amino acids, between about 200 amino acids to about 780 amino acids, between about 200 amino acids to about 770 amino acids, between about 200 amino acids to about 760 amino acids, between about 200 amino acids to about 750 amino acids, between about 200 amino acids to about 740 amino acids, between about 200 amino acids to about 730 amino acids, between about 200 amino acids to about 720 amino acids, between about 200 amino acids to about 710 amino acids, between about 200 amino acids to about 700 amino acids, between about 200 amino acids to about 690 amino acids, between about 200 amino acids to about 680 amino acids, between 30 amino acids to about 670 amino acids, between about 200 amino acids to about 660 amino acids, between about 200 amino acids to about 650 amino acids, between about 200 amino acids to about 640 amino acids, between about 30 amino acids to about 630 amino acids, between about 200 amino acids to about 620 amino acids, between about 200 amino acids to about 610 amino acids, between about 200 amino acids to about 600 amino acids, between about 200 amino acids to about 590 amino acids, between about 200 amino acids to about 580 amino acids, between about 200 amino acids to about 570 amino acids, between about 200 amino acids to about 560 amino acids, between about 200 amino acids to about 550 amino acids, between about 200 amino acids to about 540 amino acids, between about 200 amino acids to about 530 amino acids, between about 200 amino acids to about 520 amino acids, between about 200 amino acids to about 510 amino acids, between about 200 amino acids to about 500 amino acids, between about 200 amino acids to about 490 amino acids, between about 200 amino acids to about 480 amino acids, between about 200 amino acids to about 470 amino acids, between about 200 amino acids to about 460 amino acids, between about 200 amino acids to about 450 amino acids, between about 200 amino acids to about 440 amino acids, between about 200 amino acids to about 430 amino acids, between about 200 amino acids to about 420 amino acids, between about 200 amino acids to about 410 amino acids, between about 200 amino acids to about 400 amino acids, between about 200 amino acids to about 390 amino acids, between about 200 amino acids to about 380 amino acids, between about 200 amino acids to about 370 amino acids, between about 200 amino acids to about 360 amino acids, between about 200 amino acids to about 350 amino acids, between about 200 amino acids to about 340 amino acids, between about 200 amino acids to about 330 amino acids, between about 200 amino acids to about 320 amino acids, between about 200 amino acids to about 310 amino acids, between about 200 amino acids to about 300 amino acids, between about 200 amino acids to about 290 amino acids, between about 200 amino acids to about 280 amino acids, between about 200 amino acids to about 270 amino acids, between about 200 amino acids to about 260 amino acids, between about 200 amino acids to about 250 amino acids, between about 200 amino acids to about 240 amino acids, between about 200 amino acids to about 230 amino acids, between about 200 amino acids to about 220 amino acids, between about 200 amino acids to about 210 amino acids, between about 250 amino acids to about 800 amino acids, between about 250 amino acids to about 790 amino acids, between about 250 amino acids to about 780 amino acids, between about 250 amino acids to about 770 amino acids, between about 250 amino acids to about 760 amino acids, between about 250 amino acids to about 750 amino acids, between about 250 amino acids to about 740 amino acids, between about 250 amino acids to about 730 amino acids, between about 250 amino acids to about 720 amino acids, between about 250 amino acids to about 710 amino acids, between about 250 amino acids to about 700 amino acids, between about 250 amino acids to about 690 amino acids, between about 250 amino acids to about 680 amino acids, between 30 amino acids to about 670 amino acids, between about 250 amino acids to about 660 amino acids, between about 250 amino acids to about 650 amino acids, between about 250 amino acids to about 640 amino acids, between about 30 amino acids to about 630 amino acids, between about 250 amino acids to about 620 amino acids, between about 250 amino acids to about 610 amino acids, between about 250 amino acids to about 600 amino acids, between about 250 amino acids to about 590 amino acids, between about 250 amino acids to about 580 amino acids, between about 250 amino acids to about 570 amino acids, between about 250 amino acids to about 560 amino acids, between about 250 amino acids to about 550 amino acids, between about 250 amino acids to about 540 amino acids, between about 250 amino acids to about 530 amino acids, between about 250 amino acids to about 520 amino acids, between about 250 amino acids to about 510 amino acids, between about 250 amino acids to about 500 amino acids, between about 250 amino acids to about 490 amino acids, between about 250 amino acids to about 480 amino acids, between about 250 amino acids to about 470 amino acids, between about 250 amino acids to about 460 amino acids, between about 250 amino acids to about 450 amino acids, between about 250 amino acids to about 440 amino acids, between about 250 amino acids to about 430 amino acids, between about 250 amino acids to about 420 amino acids, between about 250 amino acids to about 410 amino acids, between about 250 amino acids to about 400 amino acids, between about 250 amino acids to about 390 amino acids, between about 250 amino acids to about 380 amino acids, between about 250 amino acids to about 370 amino acids, between about 250 amino acids to about 360 amino acids, between about 250 amino acids to about 350 amino acids, between about 250 amino acids to about 340 amino acids, between about 250 amino acids to about 330 amino acids, between about 250 amino acids to about 320 amino acids, between about 250 amino acids to about 310 amino acids, between about 250 amino acids to about 300 amino acids, between about 250 amino acids to about 290 amino acids, between about 250 amino acids to about 280 amino acids, between about 250 amino acids to about 270 amino acids, between about 250 amino acids to about 260 amino acids, between about 280 amino acids to about 800 amino acids, between about 280 amino acids to about 790 amino acids, between about 280 amino acids to about 780 amino acids, between about 280 amino acids to about 770 amino acids, between about 280 amino acids to about 760 amino acids, between about 280 amino acids to about 750 amino acids, between about 280 amino acids to about 740 amino acids, between about 280 amino acids to about 730 amino acids, between about 280 amino acids to about 720 amino acids, between about 280 amino acids to about 710 amino acids, between about 280 amino acids to about 700 amino acids, between about 280 amino acids to about 690 amino acids, between about 280 amino acids to about 680 amino acids, between 30 amino acids to about 670 amino acids, between about 280 amino acids to about 660 amino acids, between about 280 amino acids to about 650 amino acids, between about 280 amino acids to about 640 amino acids, between about 30 amino acids to about 630 amino acids, between about 280 amino acids to about 620 amino acids, between about 280 amino acids to about 610 amino acids, between about 280 amino acids to about 600 amino acids, between about 280 amino acids to about 590 amino acids, between about 280 amino acids to about 580 amino acids, between about 280 amino acids to about 570 amino acids, between about 280 amino acids to about 560 amino acids, between about 280 amino acids to about 550 amino acids, between about 280 amino acids to about 540 amino acids, between about 280 amino acids to about 530 amino acids, between about 280 amino acids to about 520 amino acids, between about 280 amino acids to about 510 amino acids, between about 280 amino acids to about 500 amino acids, between about 280 amino acids to about 490 amino acids, between about 280 amino acids to about 480 amino acids, between about 280 amino acids to about 470 amino acids, between about 280 amino acids to about 460 amino acids, between about 280 amino acids to about 450 amino acids, between about 280 amino acids to about 440 amino acids, between about 280 amino acids to about 430 amino acids, between about 280 amino acids to about 420 amino acids, between about 280 amino acids to about 410 amino acids, between about 280 amino acids to about 400 amino acids, between about 280 amino acids to about 390 amino acids, between about 280 amino acids to about 380 amino acids, between about 280 amino acids to about 370 amino acids, between about 280 amino acids to about 360 amino acids, between about 280 amino acids to about 350 amino acids, between about 280 amino acids to about 340 amino acids, between about 280 amino acids to about 330 amino acids, between about 280 amino acids to about 320 amino acids, between about 280 amino acids to about 310 amino acids, between about 280 amino acids to about 300 amino acids, between about 300 amino acids to about 800 amino acids, between about 300 amino acids to about 790 amino acids, between about 300 amino acids to about 780 amino acids, between about 300 amino acids to about 770 amino acids, between about 300 amino acids to about 760 amino acids, between about 300 amino acids to about 750 amino acids, between about 300 amino acids to about 740 amino acids, between about 300 amino acids to about 730 amino acids, between about 300 amino acids to about 720 amino acids, between about 300 amino acids to about 710 amino acids, between about 300 amino acids to about 700 amino acids, between about 300 amino acids to about 690 amino acids, between about 300 amino acids to about 680 amino acids, between 30 amino acids to about 670 amino acids, between about 300 amino acids to about 660 amino acids, between about 300 amino acids to about 650 amino acids, between about 300 amino acids to about 640 amino acids, between about 30 amino acids to about 630 amino acids, between about 300 amino acids to about 620 amino acids, between about 300 amino acids to about 610 amino acids, between about 300 amino acids to about 600 amino acids, between about 300 amino acids to about 590 amino acids, between about 300 amino acids to about 580 amino acids, between about 300 amino acids to about 570 amino acids, between about 300 amino acids to about 560 amino acids, between about 300 amino acids to about 550 amino acids, between about 300 amino acids to about 540 amino acids, between about 300 amino acids to about 530 amino acids, between about 300 amino acids to about 520 amino acids, between about 300 amino acids to about 510 amino acids, between about 300 amino acids to about 500 amino acids, between about 300 amino acids to about 490 amino acids, between about 300 amino acids to about 480 amino acids, between about 300 amino acids to about 470 amino acids, between about 300 amino acids to about 460 amino acids, between about 300 amino acids to about 450 amino acids, between about 300 amino acids to about 440 amino acids, between about 300 amino acids to about 430 amino acids, between about 300 amino acids to about 420 amino acids, between about 300 amino acids to about 410 amino acids, between about 300 amino acids to about 400 amino acids, between about 300 amino acids to about 390 amino acids, between about 300 amino acids to about 380 amino acids, between about 300 amino acids to about 370 amino acids, between about 300 amino acids to about 360 amino acids, between about 300 amino acids to about 350 amino acids, between about 300 amino acids to about 340 amino acids, between about 300 amino acids to about 330 amino acids, between about 300 amino acids to about 320 amino acids, between about 300 amino acids to about 310 amino acids, between about 350 amino acids to about 800 amino acids, between about 350 amino acids to about 790 amino acids, between about 350 amino acids to about 780 amino acids, between about 350 amino acids to about 770 amino acids, between about 350 amino acids to about 760 amino acids, between about 350 amino acids to about 750 amino acids, between about 350 amino acids to about 740 amino acids, between about 350 amino acids to about 730 amino acids, between about 350 amino acids to about 720 amino acids, between about 350 amino acids to about 710 amino acids, between about 350 amino acids to about 700 amino acids, between about 350 amino acids to about 690 amino acids, between about 350 amino acids to about 680 amino acids, between 30 amino acids to about 670 amino acids, between about 350 amino acids to about 660 amino acids, between about 350 amino acids to about 650 amino acids, between about 350 amino acids to about 640 amino acids, between about 30 amino acids to about 630 amino acids, between about 350 amino acids to about 620 amino acids, between about 350 amino acids to about 610 amino acids, between about 350 amino acids to about 600 amino acids, between about 350 amino acids to about 590 amino acids, between about 350 amino acids to about 580 amino acids, between about 350 amino acids to about 570 amino acids, between about 350 amino acids to about 560 amino acids, between about 350 amino acids to about 550 amino acids, between about 350 amino acids to about 540 amino acids, between about 350 amino acids to about 530 amino acids, between about 350 amino acids to about 520 amino acids, between about 350 amino acids to about 510 amino acids, between about 350 amino acids to about 500 amino acids, between about 350 amino acids to about 490 amino acids, between about 350 amino acids to about 480 amino acids, between about 350 amino acids to about 470 amino acids, between about 350 amino acids to about 460 amino acids, between about 350 amino acids to about 450 amino acids, between about 350 amino acids to about 440 amino acids, between about 350 amino acids to about 430 amino acids, between about 350 amino acids to about 420 amino acids, between about 350 amino acids to about 410 amino acids, between about 350 amino acids to about 400 amino acids, between about 350 amino acids to about 390 amino acids, between about 350 amino acids to about 380 amino acids, between about 350 amino acids to about 370 amino acids, between about 350 amino acids to about 360 amino acids, between about 380 amino acids to about 800 amino acids, between about 380 amino acids to about 790 amino acids, between about 380 amino acids to about 780 amino acids, between about 380 amino acids to about 770 amino acids, between about 380 amino acids to about 760 amino acids, between about 380 amino acids to about 750 amino acids, between about 380 amino acids to about 740 amino acids, between about 380 amino acids to about 730 amino acids, between about 380 amino acids to about 720 amino acids, between about 380 amino acids to about 710 amino acids, between about 380 amino acids to about 700 amino acids, between about 380 amino acids to about 690 amino acids, between about 380 amino acids to about 680 amino acids, between 30 amino acids to about 670 amino acids, between about 380 amino acids to about 660 amino acids, between about 380 amino acids to about 650 amino acids, between about 380 amino acids to about 640 amino acids, between about 30 amino acids to about 630 amino acids, between about 380 amino acids to about 620 amino acids, between about 380 amino acids to about 610 amino acids, between about 380 amino acids to about 600 amino acids, between about 380 amino acids to about 590 amino acids, between about 380 amino acids to about 580 amino acids, between about 380 amino acids to about 570 amino acids, between about 380 amino acids to about 560 amino acids, between about 380 amino acids to about 550 amino acids, between about 380 amino acids to about 540 amino acids, between about 380 amino acids to about 530 amino acids, between about 380 amino acids to about 520 amino acids, between about 380 amino acids to about 510 amino acids, between about 380 amino acids to about 500 amino acids, between about 380 amino acids to about 490 amino acids, between about 380 amino acids to about 480 amino acids, between about 380 amino acids to about 470 amino acids, between about 380 amino acids to about 460 amino acids, between about 380 amino acids to about 450 amino acids, between about 380 amino acids to about 440 amino acids, between about 380 amino acids to about 430 amino acids, between about 380 amino acids to about 420 amino acids, between about 380 amino acids to about 410 amino acids, between about 380 amino acids to about 400 amino acids, between about 380 amino acids to about 390 amino acids, between about 400 amino acids to about 800 amino acids, between about 400 amino acids to about 790 amino acids, between about 400 amino acids to about 780 amino acids, between about 400 amino acids to about 770 amino acids, between about 400 amino acids to about 760 amino acids, between about 400 amino acids to about 750 amino acids, between about 400 amino acids to about 740 amino acids, between about 400 amino acids to about 730 amino acids, between about 400 amino acids to about 720 amino acids, between about 400 amino acids to about 710 amino acids, between about 400 amino acids to about 700 amino acids, between about 400 amino acids to about 690 amino acids, between about 400 amino acids to about 680 amino acids, between 30 amino acids to about 670 amino acids, between about 400 amino acids to about 660 amino acids, between about 400 amino acids to about 650 amino acids, between about 400 amino acids to about 640 amino acids, between about 30 amino acids to about 630 amino acids, between about 400 amino acids to about 620 amino acids, between about 400 amino acids to about 610 amino acids, between about 400 amino acids to about 600 amino acids, between about 400 amino acids to about 590 amino acids, between about 400 amino acids to about 580 amino acids, between about 400 amino acids to about 570 amino acids, between about 400 amino acids to about 560 amino acids, between about 400 amino acids to about 550 amino acids, between about 400 amino acids to about 540 amino acids, between about 400 amino acids to about 530 amino acids, between about 400 amino acids to about 520 amino acids, between about 400 amino acids to about 510 amino acids, between about 400 amino acids to about 500 amino acids, between about 400 amino acids to about 490 amino acids, between about 400 amino acids to about 480 amino acids, between about 400 amino acids to about 470 amino acids, between about 400 amino acids to about 460 amino acids, between about 400 amino acids to about 450 amino acids, between about 400 amino acids to about 440 amino acids, between about 400 amino acids to about 430 amino acids, between about 400 amino acids to about 420 amino acids, between about 400 amino acids to about 410 amino acids, between about 450 amino acids to about 800 amino acids, between about 450 amino acids to about 790 amino acids, between about 450 amino acids to about 780 amino acids, between about 450 amino acids to about 770 amino acids, between about 450 amino acids to about 760 amino acids, between about 450 amino acids to about 750 amino acids, between about 450 amino acids to about 740 amino acids, between about 450 amino acids to about 730 amino acids, between about 450 amino acids to about 720 amino acids, between about 450 amino acids to about 710 amino acids, between about 450 amino acids to about 700 amino acids, between about 450 amino acids to about 690 amino acids, between about 450 amino acids to about 680 amino acids, between 30 amino acids to about 670 amino acids, between about 450 amino acids to about 660 amino acids, between about 450 amino acids to about 650 amino acids, between about 450 amino acids to about 640 amino acids, between about 30 amino acids to about 630 amino acids, between about 450 amino acids to about 620 amino acids, between about 450 amino acids to about 610 amino acids, between about 450 amino acids to about 600 amino acids, between about 450 amino acids to about 590 amino acids, between about 450 amino acids to about 580 amino acids, between about 450 amino acids to about 570 amino acids, between about 450 amino acids to about 560 amino acids, between about 450 amino acids to about 550 amino acids, between about 450 amino acids to about 540 amino acids, between about 450 amino acids to about 530 amino acids, between about 450 amino acids to about 520 amino acids, between about 450 amino acids to about 510 amino acids, between about 450 amino acids to about 500 amino acids, between about 450 amino acids to about 490 amino acids, between about 450 amino acids to about 480 amino acids, between about 450 amino acids to about 470 amino acids, between about 450 amino acids to about 460 amino acids, between about 480 amino acids to about 800 amino acids, between about 480 amino acids to about 790 amino acids, between about 480 amino acids to about 780 amino acids, between about 480 amino acids to about 770 amino acids, between about 480 amino acids to about 760 amino acids, between about 480 amino acids to about 750 amino acids, between about 480 amino acids to about 740 amino acids, between about 480 amino acids to about 730 amino acids, between about 480 amino acids to about 720 amino acids, between about 480 amino acids to about 710 amino acids, between about 480 amino acids to about 700 amino acids, between about 480 amino acids to about 690 amino acids, between about 480 amino acids to about 680 amino acids, between 30 amino acids to about 670 amino acids, between about 480 amino acids to about 660 amino acids, between about 480 amino acids to about 650 amino acids, between about 480 amino acids to about 640 amino acids, between about 30 amino acids to about 630 amino acids, between about 480 amino acids to about 620 amino acids, between about 480 amino acids to about 610 amino acids, between about 480 amino acids to about 600 amino acids, between about 480 amino acids to about 590 amino acids, between about 480 amino acids to about 580 amino acids, between about 480 amino acids to about 570 amino acids, between about 480 amino acids to about 560 amino acids, between about 480 amino acids to about 550 amino acids, between about 480 amino acids to about 540 amino acids, between about 480 amino acids to about 530 amino acids, between about 480 amino acids to about 520 amino acids, between about 480 amino acids to about 510 amino acids, between about 480 amino acids to about 500 amino acids, between about 480 amino acids to about 490 amino acids, between about 50 amino acids to about 800 amino acids, between about 50 amino acids to about 790 amino acids, between about 50 amino acids to about 780 amino acids, between about 50 amino acids to about 770 amino acids, between about 50 amino acids to about 760 amino acids, between about 50 amino acids to about 750 amino acids, between about 50 amino acids to about 740 amino acids, between about 50 amino acids to about 730 amino acids, between about 50 amino acids to about 720 amino acids, between about 50 amino acids to about 710 amino acids, between about 50 amino acids to about 700 amino acids, between about 50 amino acids to about 690 amino acids, between about 50 amino acids to about 680 amino acids, between 30 amino acids to about 670 amino acids, between about 50 amino acids to about 660 amino acids, between about 50 amino acids to about 650 amino acids, between about 50 amino acids to about 640 amino acids, between about 30 amino acids to about 630 amino acids, between about 50 amino acids to about 620 amino acids, between about 50 amino acids to about 610 amino acids, between about 50 amino acids to about 600 amino acids, between about 50 amino acids to about 590 amino acids, between about 50 amino acids to about 580 amino acids, between about 50 amino acids to about 570 amino acids, between about 50 amino acids to about 560 amino acids, between about 50 amino acids to about 550 amino acids, between about 50 amino acids to about 540 amino acids, between about 50 amino acids to about 530 amino acids, between about 50 amino acids to about 520 amino acids, between about 50 amino acids to about 510 amino acids, between about 550 amino acids to about 800 amino acids, between about 550 amino acids to about 790 amino acids, between about 550 amino acids to about 780 amino acids, between about 550 amino acids to about 770 amino acids, between about 550 amino acids to about 760 amino acids, between about 550 amino acids to about 750 amino acids, between about 550 amino acids to about 740 amino acids, between about 550 amino acids to about 730 amino acids, between about 550 amino acids to about 720 amino acids, between about 550 amino acids to about 710 amino acids, between about 550 amino acids to about 700 amino acids, between about 550 amino acids to about 690 amino acids, between about 550 amino acids to about 680 amino acids, between 30 amino acids to about 670 amino acids, between about 550 amino acids to about 660 amino acids, between about 550 amino acids to about 650 amino acids, between about 550 amino acids to about 640 amino acids, between about 30 amino acids to about 630 amino acids, between about 550 amino acids to about 620 amino acids, between about 550 amino acids to about 610 amino acids, between about 550 amino acids to about 600 amino acids, between about 550 amino acids to about 590 amino acids, between about 550 amino acids to about 580 amino acids, between about 550 amino acids to about 570 amino acids, between about 550 amino acids to about 560 amino acids, between about 580 amino acids to about 800 amino acids, between about 580 amino acids to about 790 amino acids, between about 580 amino acids to about 780 amino acids, between about 580 amino acids to about 770 amino acids, between about 580 amino acids to about 760 amino acids, between about 580 amino acids to about 750 amino acids, between about 580 amino acids to about 740 amino acids, between about 580 amino acids to about 730 amino acids, between about 580 amino acids to about 720 amino acids, between about 580 amino acids to about 710 amino acids, between about 580 amino acids to about 700 amino acids, between about 580 amino acids to about 690 amino acids, between about 580 amino acids to about 680 amino acids, between 30 amino acids to about 670 amino acids, between about 580 amino acids to about 660 amino acids, between about 580 amino acids to about 650 amino acids, between about 580 amino acids to about 640 amino acids, between about 30 amino acids to about 630 amino acids, between about 580 amino acids to about 620 amino acids, between about 580 amino acids to about 610 amino acids, between about 580 amino acids to about 600 amino acids, between about 580 amino acids to about 590 amino acids, between about 600 amino acids to about 800 amino acids, between about 600 amino acids to about 790 amino acids, between about 600 amino acids to about 780 amino acids, between about 600 amino acids to about 770 amino acids, between about 600 amino acids to about 760 amino acids, between about 600 amino acids to about 750 amino acids, between about 600 amino acids to about 740 amino acids, between about 600 amino acids to about 730 amino acids, between about 600 amino acids to about 720 amino acids, between about 600 amino acids to about 710 amino acids, between about 600 amino acids to about 700 amino acids, between about 600 amino acids to about 690 amino acids, between about 600 amino acids to about 680 amino acids, between 30 amino acids to about 670 amino acids, between about 600 amino acids to about 660 amino acids, between about 600 amino acids to about 650 amino acids, between about 600 amino acids to about 640 amino acids, between about 30 amino acids to about 630 amino acids, between about 600 amino acids to about 620 amino acids, between about 600 amino acids to about 610 amino acids, between about 650 amino acids to about 800 amino acids, between about 650 amino acids to about 790 amino acids, between about 650 amino acids to about 780 amino acids, between about 650 amino acids to about 770 amino acids, between about 650 amino acids to about 760 amino acids, between about 650 amino acids to about 750 amino acids, between about 650 amino acids to about 740 amino acids, between about 650 amino acids to about 730 amino acids, between about 650 amino acids to about 720 amino acids, between about 650 amino acids to about 710 amino acids, between about 650 amino acids to about 700 amino acids, between about 650 amino acids to about 690 amino acids, between about 650 amino acids to about 680 amino acids, between 30 amino acids to about 670 amino acids, between about 650 amino acids to about 660 amino acids, between about 680 amino acids to about 800 amino acids, between about 680 amino acids to about 790 amino acids, between about 680 amino acids to about 780 amino acids, between about 680 amino acids to about 770 amino acids, between about 680 amino acids to about 760 amino acids, between about 680 amino acids to about 750 amino acids, between about 680 amino acids to about 740 amino acids, between about 680 amino acids to about 730 amino acids, between about 680 amino acids to about 720 amino acids, between about 680 amino acids to about 710 amino acids, between about 680 amino acids to about 700 amino acids, between about 680 amino acids to about 690 amino acids, between about 700 amino acids to about 800 amino acids, between about 700 amino acids to about 790 amino acids, between about 700 amino acids to about 780 amino acids, between about 700 amino acids to about 770 amino acids, between about 700 amino acids to about 760 amino acids, between about 700 amino acids to about 750 amino acids, between about 700 amino acids to about 740 amino acids, between about 700 amino acids to about 730 amino acids, between about 700 amino acids to about 720 amino acids, between about 700 amino acids to about 710 amino acids, between about 750 amino acids to about 800 amino acids, between about 750 amino acids to about 790 amino acids, between about 750 amino acids to about 780 amino acids, between about 750 amino acids to about 770 amino acids, between about 750 amino acids to about 760 amino acids, or between about 780 amino acids to about 800 amino acids) in length. In some examples, the second vector further includes a poly(A) sequence.

In some examples where the number of different vectors in the composition is two, the N-terminal portion encoded by one of the two vectors can include a portion including amino acid position 1 to about amino acid position 800, about amino acid position 790, about amino acid position 780, about amino acid position 770, about amino acid position 760, about amino acid position 750, about amino acid position 740, about amino acid position 730, about amino acid position 720, about amino acid position 710, about amino acid position 700, about amino acid position 690, about amino acid position 680, about amino acid position 670, about amino acid position 660, about amino acid position 650, about amino acid position 640, about amino acid position 630, about amino acid position 620, about amino acid position 610, about amino acid position 600, about amino acid position 590, about amino acid position 580, about amino acid position 570, about amino acid position 560, about amino acid position 550, about amino acid position 540, about amino acid position 530, about amino acid position 520, about amino acid position 510, about amino acid position 500, about amino acid position 490, about amino acid position 480, about amino acid position 470, about amino acid position 460, about amino acid position 450, about amino acid position 440, about amino acid position 430, about amino acid position 420, about amino acid position 410, about amino acid position 400, about amino acid position 390, about amino acid position 380, about amino acid position 370, about amino acid position 360, about amino acid position 350, about amino acid position 340, about amino acid position 330, about amino acid position 320, about amino acid position 310, about amino acid position 300, about amino acid position 290, about amino acid position 280, about amino acid position 270, about amino acid position 260, about amino acid position 250, about amino acid position 240, about amino acid position 230, about amino acid position 220, about amino acid position 210, about amino acid position 200, about amino acid position 190, about amino acid position 180, about amino acid position 170, about amino acid position 160, about amino acid position 150, about amino acid position 140, about amino acid position 130, about amino acid position 120, about amino acid position 110, about amino acid position 100, about amino acid position 90, about amino acid position 80, about amino acid position 70, about amino acid position 60, about amino acid position 50, or about amino acid position 40 of a wildtype hair cell target protein (e.g., SEQ ID NO: 8, SEQ ID NO: 9, SEQ ID NO: 10, SEQ ID NO: 11, SEQ ID NO: 12, SEQ ID NO: 20, SEQ ID NO: 23, SEQ ID NO: 24, SEQ ID NO: 25, SEQ ID NO: 26, SEQ ID NO: 27, SEQ ID NO: 28, SEQ ID NO: 30, SEQ ID NO: 31, SEQ ID NO: 32, SEQ ID NO: 34, SEQ ID NO: 39, SEQ ID NO: 41, SEQ ID NO: 43, SEQ ID NO: 45, SEQ ID NO: 48, SEQ ID NO: 51, SEQ ID NO: 53, SEQ ID NO: 55, SEQ ID NO: 57, SEQ ID NO: 59, SEQ ID NO: 61, SEQ ID NO: 64, SEQ ID NO: 66, SEQ ID NO: 68, SEQ ID NO: 69, SEQ ID NO: 70, SEQ ID NO: 72, SEQ ID NO: 74, SEQ ID NO: 76, SEQ ID NO: 77, SEQ ID NO: 78, or SEQ ID NO: 79).

In some examples where the number of different vectors in the composition is two, the N-terminal portion of the precursor hair cell target protein can include a portion including amino acid position 1 to amino acid position 300, amino acid position 1 to amino acid position 310, amino acid position 1 to about amino acid position 320, amino acid position 1 to about amino acid position 330, amino acid position 1 to about amino acid position 340, amino acid position 1 to about amino acid position 350, amino acid position 1 to about amino acid position 360, amino acid position 1 to about amino acid position 370, amino acid position 1 to about amino acid position 380, amino acid position 1 to about amino acid position 390, amino acid position 1 to about amino acid position 400, amino acid position 1 to about amino acid position 410, amino acid position 1 to about amino acid position 420, amino acid position 1 to about amino acid position 430, amino acid position 1 to about amino acid position 440, amino acid position 1 to about amino acid position 450, amino acid position 1 to about amino acid position 460, amino acid position 1 to about amino acid position 470, amino acid position 1 to about amino acid position 480, amino acid position 1 to about amino acid position 490, amino acid position 1 to about amino acid position 500, amino acid position 1 to about amino acid position 510, amino acid position 1 to about amino acid position 520, amino acid position 1 to about amino acid position 530, amino acid position 1 to about amino acid position 540, amino acid position 1 to about amino acid position 550, amino acid position 1 to about amino acid position 560, amino acid position 1 to about amino acid position 570, amino acid position 1 to about amino acid position 580, amino acid position 1 to about amino acid position 590, amino acid position 1 to about amino acid position 600, amino acid position 1 to about amino acid position 610, amino acid position 1 to about amino acid position 620, amino acid position 1 to about amino acid position 630, amino acid position 1 to about amino acid position 640, amino acid position 1 to about amino acid position 650, amino acid position 1 to about amino acid position 660, amino acid position 1 to about amino acid position 670, amino acid position 1 to about amino acid position 680, amino acid position 1 to about amino acid position 690, amino acid position 1 to about amino acid position 700, amino acid position 1 to about amino acid position 710, amino acid position 1 to about amino acid position 720, amino acid position 1 to about amino acid position 730, amino acid position 1 to about amino acid position 740, amino acid position 1 to about amino acid position 750, amino acid position 1 to about amino acid position 760, amino acid position 1 to about amino acid position 770, amino acid position 1 to about amino acid position 780, amino acid position 1 to about amino acid position 790, amino acid position 1 to about amino acid position 800 of a wildtype hair cell target protein (e.g., SEQ ID NO: 8, SEQ ID NO: 9, SEQ ID NO: 10, SEQ ID NO: 11, SEQ ID NO: 12, SEQ ID NO: 20, SEQ ID NO: 23, SEQ ID NO: 24, SEQ ID NO: 25, SEQ ID NO: 26, SEQ ID NO: 27, SEQ ID NO: 28, SEQ ID NO: 30, SEQ ID NO: 31, SEQ ID NO: 32, SEQ ID NO: 34, SEQ ID NO: 35, SEQ ID NO: 41, SEQ ID NO: 43, SEQ ID NO: 45, SEQ ID NO: 48, SEQ ID NO: 51, SEQ ID NO: 53, SEQ ID NO: 55, SEQ ID NO: 57, SEQ ID NO: 59, SEQ ID NO: 61, SEQ ID NO: 64, SEQ ID NO: 66, SEQ ID NO: 68, SEQ ID NO: 69, SEQ ID NO: 70, SEQ ID NO: 72, SEQ ID NO: 74, SEQ ID NO: 76, SEQ ID NO: 77, SEQ ID NO: 78, SEQ ID NO: 79).

In some examples where the number of different vectors in the composition is two, the N-terminal portion encoded by one of the two vectors can include a portion including amino acid position 1 to about amino acid position 800, about amino acid position 790, about amino acid position 780, about amino acid position 770, about amino acid position 760, about amino acid position 750, about amino acid position 740, about amino acid position 730, about amino acid position 720, about amino acid position 710, about amino acid position 700, about amino acid position 690, about amino acid position 680, about amino acid position 670, about amino acid position 660, about amino acid position 650, about amino acid position 640, about amino acid position 630, about amino acid position 620, about amino acid position 610, about amino acid position 600, about amino acid position 590, about amino acid position 580, about amino acid position 570, about amino acid position 560, about amino acid position 550, about amino acid position 540, about amino acid position 530, about amino acid position 520, about amino acid position 510, about amino acid position 500, about amino acid position 490, about amino acid position 480, about amino acid position 470, about amino acid position 460, about amino acid position 450, about amino acid position 440, about amino acid position 430, about amino acid position 420, about amino acid position 410, about amino acid position 400, about amino acid position 390, about amino acid position 380, about amino acid position 370, about amino acid position 360, about amino acid position 350, about amino acid position 340, about amino acid position 330, about amino acid position 320, about amino acid position 310, about amino acid position 300, about amino acid position 290, about amino acid position 280, about amino acid position 270, about amino acid position 260, about amino acid position 250, about amino acid position 240, about amino acid position 230, about amino acid position 220, about amino acid position 210, about amino acid position 200, about amino acid position 190, about amino acid position 180, about amino acid position 170, about amino acid position 160, about amino acid position 150, about amino acid position 140, about amino acid position 130, about amino acid position 120, about amino acid position 110, about amino acid position 100, about amino acid position 90, about amino acid position 80, about amino acid position 70, about amino acid position 60, about amino acid position 50, or about amino acid position 40 of a wildtype supporting cell target protein (e.g., SEQ ID NO: 1 or SEQ ID NO: 4).

In some examples where the number of different vectors in the composition is two, the N-terminal portion of the precursor supporting cell target protein can include a portion including amino acid position 1 to amino acid position 300, amino acid position 1 to amino acid position 310, amino acid position 1 to about amino acid position 320, amino acid position 1 to about amino acid position 330, amino acid position 1 to about amino acid position 340, amino acid position 1 to about amino acid position 350, amino acid position 1 to about amino acid position 360, amino acid position 1 to about amino acid position 370, amino acid position 1 to about amino acid position 380, amino acid position 1 to about amino acid position 390, amino acid position 1 to about amino acid position 400, amino acid position 1 to about amino acid position 410, amino acid position 1 to about amino acid position 420, amino acid position 1 to about amino acid position 430, amino acid position 1 to about amino acid position 440, amino acid position 1 to about amino acid position 450, amino acid position 1 to about amino acid position 460, amino acid position 1 to about amino acid position 470, amino acid position 1 to about amino acid position 480, amino acid position 1 to about amino acid position 490, amino acid position 1 to about amino acid position 500, amino acid position 1 to about amino acid position 510, amino acid position 1 to about amino acid position 520, amino acid position 1 to about amino acid position 530, amino acid position 1 to about amino acid position 540, amino acid position 1 to about amino acid position 550, amino acid position 1 to about amino acid position 560, amino acid position 1 to about amino acid position 570, amino acid position 1 to about amino acid position 580, amino acid position 1 to about amino acid position 590, amino acid position 1 to about amino acid position 600, amino acid position 1 to about amino acid position 610, amino acid position 1 to about amino acid position 620, amino acid position 1 to about amino acid position 630, amino acid position 1 to about amino acid position 640, amino acid position 1 to about amino acid position 650, amino acid position 1 to about amino acid position 660, amino acid position 1 to about amino acid position 670, amino acid position 1 to about amino acid position 680, amino acid position 1 to about amino acid position 690, amino acid position 1 to about amino acid position 700, amino acid position 1 to about amino acid position 710, amino acid position 1 to about amino acid position 720, amino acid position 1 to about amino acid position 730, amino acid position 1 to about amino acid position 740, amino acid position 1 to about amino acid position 750, amino acid position 1 to about amino acid position 760, amino acid position 1 to about amino acid position 770, amino acid position 1 to about amino acid position 780, amino acid position 1 to about amino acid position 790, amino acid position 1 to about amino acid position 800 of a wildtype supporting cell target protein (e.g., SEQ ID NO: 1 or SEQ ID NO: 4).

As used herein, the term “vector” means a composition including a polynucleotide capable of carrying at least one exogenous nucleic acid fragment, e.g., a plasmid vector, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector (e.g., any adenoviral vectors (e.g., pSV or pCMV vectors), any retroviral vectors as described herein) and any Gateway® vectors. A vector can, e.g., include sufficient cis-acting elements for expression; other elements for expression can be supplied by the host primate cell or in an in vitro expression system. The term “vector” includes any genetic element (e.g., a plasmid, a transposon, a cosmid, an artificial chromosome, or a viral vector, etc.) that is capable of replicating when associated with the proper control elements. Thus, the term includes cloning and expression vectors, as well as viral vectors (e.g., an adeno-associated virus (AAV) vector, an adenovirus vector, a lentivirus vector, or a retrovirus vector).

Vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Skilled practitioners will be capable of selecting suitable vectors and primate cells for making any of the nucleic acids described herein.

In some embodiments the vector is a plasmid (i.e. a circular DNA molecule that can autonomously replicate inside a cell). In some embodiments, the vector can be a cosmid (e.g., pWE and sCos series (Wahl et al. (1987), Evans et al. (1989)).

In some embodiments, the vector(s) is a viral vector (e.g., adeno-associated virus, adenovirus, lentivirus, and retrovirus). Non-limiting examples of viral vectors are described herein. In some embodiments, the vector(s) is an adeno-associated viral vector (AAV) (see, e.g., Asokan et al., Mol. Ther. 20: 699-7080, 2012). “Recombinant AAV vectors” or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., an outer hair cell).

The AAV sequences of the vector typically include the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein are flanked by 5′ and 3′ AAV ITR sequences in the AAV vectors. The AAV ITR sequences may be obtained from any known AAV, including presently identified AAV types.

AAV vectors as described herein may include any of the regulatory elements described herein (e.g., one or more of a promoter, a polyA sequence, and an IRES).

In some embodiments, the AAV vector is selected from the group consisting of: an AAV1 vector, an AAV2 vector, an AAV3 vector, an AAV4 vector, an AAV5 vector, an AAV6 vector, an AAV7 vector, an AAV8 vector, an AAV9 vector, an AAV2.7m8 vector, an AAV8BP2 vector, and an AAV293 vector. Additional exemplary AAV vectors that can be used herein are known in the art. See, e.g., Kanaan et al., Mol. Ther. Nucleic Acids 8:184-197, 2017; Li et al., Mol. Ther. 16(7): 1252-1260; Adachi et al., Nat. Commun. 5: 3075, 2014; Isgrig et al., Nat. Commun. 10(1): 427, 2019; and Gao et al., J. Virol. 78(12): 6381-6388.

In some embodiments, the vector(s) is an adenovirus (see, e.g., Dmitriev et al. (1998) J. Virol. 72: 9706-9713; and Poulin et al., J. Virol 8: 10074-10086, 2010). In some embodiments, the vector(s) is a retrovirus (see, e.g., Maier et al. (2010) Future Microbiol 5: 1507-23).

The vectors provided herein can be of different sizes. The choice of vector that is used in any of the compositions, kits, and methods described herein may depend on the size of the vector.

In some embodiments, the vector(s) is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, the vector(s) is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, the vector(s) is a viral vector and can have a total number of nucleotides of up to 10 kb. In some embodiments, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, the vector(s) is a lentivirus and can have a total number of nucleotides of up to 8 kb. In some examples, the lentivirus(es) can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some embodiments, the vector(s) is an adenovirus and can have a total number of nucleotides of up to 8 kb. In some embodiments, the adenovirus(es) can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kh, about 6 kb to about 8 kb, or about 7 kb to about 8 kb.

In some embodiments, the vector(s) is an adeno-associated virus (AAV vector) and can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments of any of the compositions, kits, and methods provided herein, the at least two different vectors can be substantially the same type of vector and may differ in size. In some embodiments, the at least two different vectors can be different types of vector, and may have substantially the same size or have different sizes.

In some embodiments, any of the at least two vectors can have a total number of nucleotides in the range of about 500 nucleotides to about 10,000 nucleotides, about 500 nucleotides to about 9,500 nucleotides, about 500 nucleotides to about 9,000 nucleotides, about 500 nucleotides to about 8,500 nucleotides, about 500 nucleotides to about 8,000 nucleotides, about 500 nucleotides to about 7,800 nucleotides, about 500 nucleotides to about 7,600 nucleotides, about 500 nucleotides to about 7,400 nucleotides, about 500 nucleotides to about 7,200 nucleotides, about 500 nucleotides to about 7,000 nucleotides, about 500 nucleotides to about 6,800 nucleotides, about 500 nucleotides to about 6,600 nucleotides, about 500 nucleotides to about 6,400 nucleotides, about 500 nucleotides to about 6,200 nucleotides, about 500 nucleotides to about 6,000 nucleotides, about 500 nucleotides to about 5,800 nucleotides, about 500 nucleotides to about 5,600 nucleotides, about 500 nucleotides to about 5,400 nucleotides, about 500 nucleotides to about 5,200 nucleotides, about 500 nucleotides to about 5,000 nucleotides, about 500 nucleotides to about 4,800 nucleotides, about 4,600 nucleotides, about 500 nucleotides to about 4,400 nucleotides, about 500 nucleotides to about 4,200 nucleotides, about 500 nucleotides to about 4,000 nucleotides, about 500 nucleotides to about 3,800 nucleotides, about 500 nucleotides to about 3,600 nucleotides, about 500 nucleotides to about 3,400 nucleotides, about 500 nucleotides to about 3,200 nucleotides, about 500 nucleotides to about 3,000 nucleotides, about 500 nucleotides to about 2,800 nucleotides, about 500 nucleotides to about 2,600 nucleotides, about 500 nucleotides to about 2,400 nucleotides, about 500 nucleotides to about 2,200 nucleotides, about 500 nucleotides to about 2,000 nucleotides, about 500 nucleotides to about 1,800 nucleotides, about 500 nucleotides to about 1,600 nucleotides, about 500 nucleotides to about 1,400 nucleotides, about 500 nucleotides to about 1,200 nucleotides, about 500 nucleotides to about 1,000 nucleotides, about 500 nucleotides to about 800 nucleotides, about 800 nucleotides to about 10,000 nucleotides, about 800 nucleotides to about 9,500 nucleotides, about 800 nucleotides to about 9,000 nucleotides, about 800 nucleotides to about 8,500 nucleotides, about 800 nucleotides to about 8,000 nucleotides, about 800 nucleotides to about 7,800 nucleotides, about 800 nucleotides to about 7,600 nucleotides, about 800 nucleotides to about 7,400 nucleotides, about 800 nucleotides to about 7,200 nucleotides, about 800 nucleotides to about 7,000 nucleotides, about 800 nucleotides to about 6,800 nucleotides, about 800 nucleotides to about 6,600 nucleotides, about 800 nucleotides to about 6,400 nucleotides, about 800 nucleotides to about 6,200 nucleotides, about 800 nucleotides to about 6,000 nucleotides, about 800 nucleotides to about 5,800 nucleotides, about 800 nucleotides to about 5,600 nucleotides, about 800 nucleotides to about 5,400 nucleotides, about 800 nucleotides to about 5,200 nucleotides, about 800 nucleotides to about 5,000 nucleotides, about 800 nucleotides to about 4,800 nucleotides, about 800 nucleotides to about 4,600 nucleotides, about 800 nucleotides to about 4,400 nucleotides, about 800 nucleotides to about 4,200 nucleotides, about 800 nucleotides to about 4,000 nucleotides, about 800 nucleotides to about 3,800 nucleotides, about 800 nucleotides to about 3,600 nucleotides, about 800 nucleotides to about 3,400 nucleotides, about 800 nucleotides to about 3,200 nucleotides, about 800 nucleotides to about 3,000 nucleotides, about 800 nucleotides to about 2,800 nucleotides, about 800 nucleotides to about 2,600 nucleotides, about 800 nucleotides to about 2,400 nucleotides, about 800 nucleotides to about 2,200 nucleotides, about 800 nucleotides to about 2,000 nucleotides, about 800 nucleotides to about 1,800 nucleotides, about 800 nucleotides to about 1,600 nucleotides, about 800 nucleotides to about 1,400 nucleotides, about 800 nucleotides to about 1,200 nucleotides, about 800 nucleotides to about 1,000 nucleotides, about 1,000 nucleotides to about 10,000 nucleotides, about 1,000 nucleotides to about 9,000 nucleotides, about 1,000 nucleotides to about 8,500 nucleotides, about 1,000 nucleotides to about 8,000 nucleotides, about 1,000 nucleotides to about 7,800 nucleotides, about 1,000 nucleotides to about 7,600 nucleotides, about 1,000 nucleotides to about 7,400 nucleotides, about 1,000 nucleotides to about 7,200 nucleotides, about 1,000 nucleotides to about 7,000 nucleotides, about 1,000 nucleotides to about 6,800 nucleotides, about 1,000 nucleotides to about 6,600 nucleotides, about 1,000 nucleotides to about 6,400 nucleotides, about 1,000 nucleotides to about 6,200 nucleotides, about 1,000 nucleotides to about 6,000 nucleotides, about 1,000 nucleotides to about 5,800 nucleotides, about 1,000 nucleotides to about 5,600 nucleotides, about 1,000 nucleotides to about 5,400 nucleotides, about 1,000 nucleotides to about 5,200 nucleotides, about 1,000 nucleotides to about 5,000 nucleotides, about 1,000 nucleotides to about 4,800 nucleotides, about 1,000 nucleotides to about 4,600 nucleotides, about 1,000 nucleotides to about 4,400 nucleotides, about 1,000 nucleotides to about 4,200 nucleotides, about 1,000 nucleotides to about 4,000 nucleotides, about 1,000 nucleotides to about 3,800 nucleotides, about 1,000 nucleotides to about 3,600 nucleotides, about 1,000 nucleotides to about 3,400 nucleotides, about 1,000 nucleotides to about 3,200 nucleotides, about 1,000 nucleotides to about 3,000 nucleotides, about 1,000 nucleotides to about 2,600 nucleotides, about 1,000 nucleotides to about 2,400 nucleotides, about 1,000 nucleotides to about 2,200 nucleotides, about 1,000 nucleotides to about 2,000 nucleotides, about 1,000 nucleotides to about 1,800 nucleotides, about 1,000 nucleotides to about 1,600 nucleotides, about 1,000 nucleotides to about 1,400 nucleotides, about 1,000 nucleotides to about 1,200 nucleotides, about 1,200 nucleotides to about 10,000 nucleotides, about 1,200 nucleotides to about 9,500 nucleotides, about 1,200 nucleotides to about 9,000 nucleotides, about 1,200 nucleotides to about 8,500 nucleotides, about 1,200 nucleotides to about 8,000 nucleotides, about 1,200 nucleotides to about 7,800 nucleotides, about 1,200 nucleotides to about 7,600 nucleotides, about 1,200 nucleotides to about 7,400 nucleotides, about 1,200 nucleotides to about 7,200 nucleotides, about 1,200 nucleotides to about 7,000 nucleotides, about 1,200 nucleotides to about 6,800 nucleotides, about 1,200 nucleotides to about 6,600 nucleotides, about 1,200 nucleotides to about 6,400 nucleotides, about 1,200 nucleotides to about 6,200 nucleotides, about 1,200 nucleotides to about 6,000 nucleotides, about 1,200 nucleotides to about 5,800 nucleotides, about 1,200 nucleotides to about 5,600 nucleotides, about 1,200 nucleotides to about 5,400 nucleotides, about 1,200 nucleotides to about 5,000 nucleotides, about 1,200 nucleotides to about 4,800 nucleotides, about 1,200 nucleotides to about 4,600 nucleotides, about 1,200 nucleotides to about 4,400 nucleotides, about 1,200 nucleotides to about 4,200 nucleotides, about 1,200 nucleotides to about 4,000 nucleotides, about 1,200 nucleotides to about 3,800 nucleotides, about 1,200 nucleotides to about 3,600 nucleotides, about 1,200 nucleotides to about 3,400 nucleotides, about 1,200 nucleotides to about 3,200 nucleotides, about 1,200 nucleotides to about 3,000 nucleotides, about 1,200 nucleotides to about 2,800 nucleotides, about 1,200 nucleotides to about 2,600 nucleotides, about 1,200 nucleotides to about 2,400 nucleotides, about 1,200 nucleotides to about 2,200 nucleotides, about 1,200 nucleotides to about 2,000 nucleotides, about 1,200 nucleotides to about 1,800 nucleotides, about 1,200 nucleotides to about 1,600 nucleotides, about 1,200 nucleotides to about 1,400 nucleotides, about 1,400 nucleotides to about 10,000 nucleotides, about 1,400 nucleotides to about 9,500 nucleotides, about 1,400 nucleotides to about 9,000 nucleotides, about 1,400 nucleotides to about 8,500 nucleotides, about 1,400 nucleotides to about 8,000 nucleotides, about 1,400 nucleotides to about 7,800 nucleotides, about 1,400 nucleotides to about 7,600 nucleotides, about 1,400 nucleotides to about 7,400 nucleotides, about 1,400 nucleotides to about 7,200 nucleotides, about 1,400 nucleotides to about 7,000 nucleotides, about 1,400 nucleotides to about 6,800 nucleotides, about 1,400 nucleotides to about 6,600 nucleotides, about 1,400 nucleotides to about 6,400 nucleotides, about 1,400 nucleotides to about 6,200 nucleotides, about 1,400 nucleotides to about 6,000 nucleotides, about 1,400 nucleotides to about 5,800 nucleotides, about 1,400 nucleotides to about 5,600 nucleotides, about 1,400 nucleotides to about 5,400 nucleotides, about 1,400 nucleotides to about 5,200 nucleotides, about 1,400 nucleotides to about 5,000 nucleotides, about 1,400 nucleotides to about 4,800 nucleotides, about 1,400 nucleotides to about 4,600 nucleotides, about 1,400 nucleotides to about 4,400 nucleotides, about 1,400 nucleotides to about 4,200 nucleotides, about 1,400 nucleotides to about 4,000 nucleotides, about 1,400 nucleotides to about 3,800 nucleotides, about 1,400 nucleotides to about 3,600 nucleotides, about 1,400 nucleotides to about 3,400 nucleotides, about 1,400 nucleotides to about 3,200 nucleotides, about 1,400 nucleotides to about 3,000 nucleotides, about 1,400 nucleotides to about 2,600 nucleotides, about 1,400 nucleotides to about 2,400 nucleotides, about 1,400 nucleotides to about 2,200 nucleotides, about 1,400 nucleotides to about 2,000 nucleotides, about 1,400 nucleotides to about 1,800 nucleotides, about 1,400 nucleotides to about 1,600 nucleotides, about 1,600 nucleotides to about 10,000 nucleotides, about 1,600 nucleotides to about 9,500 nucleotides, about 1,600 nucleotides to about 9,000 nucleotides, about 1,600 nucleotides to about 8,500 nucleotides, about 1,600 nucleotides to about 8,000 nucleotides, about 1,600 nucleotides to about 7,800 nucleotides, about 1,600 nucleotides to about 7,600 nucleotides, about 1,600 nucleotides to about 7,400 nucleotides, about 1,600 nucleotides to about 7,200 nucleotides, about 1,600 nucleotides to about 7,000 nucleotides, about 1,600 nucleotides to about 6,800 nucleotides, about 1,600 nucleotides to about 6,400 nucleotides, about 1,600 nucleotides to about 6,200 nucleotides, about 1,600 nucleotides to about 6,000 nucleotides, about 1,600 nucleotides to about 5,800 nucleotides, about 1,600 nucleotides to about 5,600 nucleotides, about 1,600 nucleotides to about 5,400 nucleotides, about 1,600 nucleotides to about 5,200 nucleotides, about 1,600 nucleotides to about 5,000 nucleotides, about 1,600 nucleotides to about 4,800 nucleotides, about 1,600 nucleotides to about 4,600 nucleotides, about 1,600 nucleotides to about 4,400 nucleotides, about 1,600 nucleotides to about 4,200 nucleotides, about 1,600 nucleotides to about 4,000 nucleotides, about 1,600 nucleotides to about 3,800 nucleotides, about 1,600 nucleotides to about 3,600 nucleotides, about 1,600 nucleotides to about 3,400 nucleotides, about 1,600 nucleotides to about 3,200 nucleotides, about 1,600 nucleotides to about 3,000 nucleotides, about 1,600 nucleotides to about 2,800 nucleotides, about 1,600 nucleotides to about 2,600 nucleotides, about 1,600 nucleotides to about 2,400 nucleotides, about 1,600 nucleotides to about 2,200 nucleotides, about 1,600 nucleotides to about 2,000 nucleotides, about 1,600 nucleotides to about 1,800 nucleotides, about 1,800 nucleotides to about 10,000 nucleotides, about 1,800 nucleotides to about 9,500 nucleotides, about 1,800 nucleotides to about 9,000 nucleotides, about 1,800 nucleotides to about 8,500 nucleotides, about 1,800 nucleotides to about 8,000 nucleotides, about 1,800 nucleotides to about 7,800 nucleotides, about 1,800 nucleotides to about 7,600 nucleotides, about 1,800 nucleotides to about 7,400 nucleotides, about 1,800 nucleotides to about 7,200 nucleotides, about 1,800 nucleotides to about 7,000 nucleotides, about 1,800 nucleotides to about 6,800 nucleotides, about 1,800 nucleotides to about 6,600 nucleotides, about 1,800 nucleotides to about 6,400 nucleotides, about 1,800 nucleotides to about 6,200 nucleotides, about 1,800 nucleotides to about 6,000 nucleotides, about 1,800 nucleotides to about 5,800 nucleotides, about 1,800 nucleotides to about 5,600 nucleotides, about 1,800 nucleotides to about 5,400 nucleotides, about 1,800 nucleotides to about 5,200 nucleotides, about 1,800 nucleotides to about 5,000 nucleotides, about 1,800 nucleotides to about 4,800 nucleotides, about 1,800 nucleotides to about 4,600 nucleotides, about 1,800 nucleotides to about 4,400 nucleotides, about 1,800 nucleotides to about 4,200 nucleotides, about 1,800 nucleotides to about 4,000 nucleotides, about 1,800 nucleotides to about 3,800 nucleotides, about 1,800 nucleotides to about 3,600 nucleotides, about 1,800 nucleotides to about 3,400 nucleotides, about 1,800 nucleotides to about 3,200 nucleotides, about 1,800 nucleotides to about 3,000 nucleotides, about 1,800 nucleotides to about 2,800 nucleotides, about 1,800 nucleotides to about 2,600 nucleotides, about 1,800 nucleotides to about 2,400 nucleotides, about 1,800 nucleotides to about 2,200 nucleotides, about 1,800 nucleotides to about 2,000 nucleotides, about 2,000 nucleotides to about 10,000 nucleotides, about 2,000 nucleotides to about 9,500 nucleotides, about 2,000 nucleotides to about 9,000 nucleotides, about 2,000 nucleotides to about 8,500 nucleotides, about 2,000 nucleotides to about 8,000 nucleotides, about 2,000 nucleotides to about 7,800 nucleotides, about 2,000 nucleotides to about 7,600 nucleotides, about 2,000 nucleotides to about 7,400 nucleotides, about 2,000 nucleotides to about 7,200 nucleotides, about 2,000 nucleotides to about 7,000 nucleotides, about 2,000 nucleotides to about 6,800 nucleotides, about 2,000 nucleotides to about 6,600 nucleotides, about 2,000 nucleotides to about 6,400 nucleotides, about 2,000 nucleotides to about 6,200 nucleotides, about 2,000 nucleotides to about 6,000 nucleotides, about 2,000 nucleotides to about 5,800 nucleotides, about 2,000 nucleotides to about 5,600 nucleotides, about 2,000 nucleotides to about 5,400 nucleotides, about 2,000 nucleotides to about 5,200 nucleotides, about 2,000 nucleotides to about 5,000 nucleotides, about 2,000 nucleotides to about 4,800 nucleotides, about 2,000 nucleotides to about 4,600 nucleotides, about 2,000 nucleotides to about 4,400 nucleotides, about 2,000 nucleotides to about 4,200 nucleotides, about 2,000 nucleotides to about 4,000 nucleotides, about 2,000 nucleotides to about 3,800 nucleotides, about 2,000 nucleotides to about 3,600 nucleotides, about 2,000 nucleotides to about 3,400 nucleotides, about 2,000 nucleotides to about 3,200 nucleotides, about 2,000 nucleotides to about 3,000 nucleotides, about 2,000 nucleotides to about 2,800 nucleotides, about 2,000 nucleotides to about 2,600 nucleotides, about 2,000 nucleotides to about 2,400 nucleotides, about 2,000 nucleotides to about 2,200 nucleotides, about 2,200 nucleotides to about 10,000 nucleotides, about 9,500 nucleotides, about 9,000 nucleotides, about 8,500 nucleotides, about 8,000 nucleotides, about 7,800 nucleotides, about 7,600 nucleotides, about 7,400 nucleotides, about 7,200 nucleotides, about 7,000 nucleotides, about 6,800 nucleotides, about 6,600 nucleotides, about 6,400 nucleotides, about 6,200 nucleotides, about 6,000 nucleotides, about 5,800 nucleotides, about 5,600 nucleotides, about 5,400 nucleotides, about 5,200 nucleotides, about 5,000 nucleotides, about 4,800 nucleotides, about 4,600 nucleotides, about 4,400 nucleotides, about 4,200 nucleotides, about 4,000 nucleotides, about 3,800 nucleotides, about 3,600 nucleotides, about 3,400 nucleotides, about 3,200 nucleotides, about 3,000 nucleotides, about 2,800 nucleotides, about 2,600 nucleotides, about 2,400 nucleotides, about 2,400 nucleotides to about 10,000 nucleotides, about 2,400 nucleotides to about 9,500 nucleotides, about 2,400 nucleotides to about 9,000 nucleotides, about 2,400 nucleotides to about 8,500 nucleotides, about 2,400 nucleotides to about 8,000 nucleotides, about 2,400 nucleotides to about 7,800 nucleotides, about 2,400 nucleotides to about 7,600 nucleotides, about 2,400 nucleotides to about 7,400 nucleotides, about 2,400 nucleotides to about 7,200 nucleotides, about 2,400 nucleotides to about 7,000 nucleotides, about 2,400 nucleotides to about 6,800 nucleotides, about 2,400 nucleotides to about 6,600 nucleotides, about 2,400 nucleotides to about 6,400 nucleotides, about 2,400 nucleotides to about 6,200 nucleotides, about 2,400 nucleotides to about 6,000 nucleotides, about 2,400 nucleotides to about 5,800 nucleotides, about 2,400 nucleotides to about 5,600 nucleotides, about 2,400 nucleotides to about 5,400 nucleotides, about 2,400 nucleotides to about 5,200 nucleotides, about 2,400 nucleotides to about 5,000 nucleotides, about 2,400 nucleotides to about 4,800 nucleotides, about 2,400 nucleotides to about 4,600 nucleotides, about 2,400 nucleotides to about 4,400 nucleotides, about 2,400 nucleotides to about 4,200 nucleotides, about 2,400 nucleotides to about 4,000 nucleotides, about 2,400 nucleotides to about 3,800 nucleotides, about 2,400 nucleotides to about 3,600 nucleotides, about 2,400 nucleotides to about 3,400 nucleotides, about 2,400 nucleotides to about 3,200 nucleotides, about 2,400 nucleotides to about 3,000 nucleotides, about 2,400 nucleotides to about 2,800 nucleotides, about 2,400 nucleotides to about 2,600 nucleotides, about 2,600 nucleotides to about 10,000 nucleotides, about 2,600 nucleotides to about 9,500 nucleotides, about 2,600 nucleotides to about 9,000 nucleotides, about 2,600 nucleotides to about 8,500 nucleotides, about 2,600 nucleotides to about 8,000 nucleotides, about 2,600 nucleotides to about 7,800 nucleotides, about 2,600 nucleotides to about 7,600 nucleotides, about 2,600 nucleotides to about 7,400 nucleotides, about 2,600 nucleotides to about 7,200 nucleotides, about 2,600 nucleotides to about 7,000 nucleotides, about 2,600 nucleotides to about 6,800 nucleotides, about 2,600 nucleotides to about 6,600 nucleotides, about 2,600 nucleotides to about 6,400 nucleotides, about 2,600 nucleotides to about 6,200 nucleotides, about 2,600 nucleotides to about 6,000 nucleotides, about 2,600 nucleotides to about 5,800 nucleotides, about 2,600 nucleotides to about 5,600 nucleotides, about 2,600 nucleotides to about 5,400 nucleotides, about 2,600 nucleotides to about 5,200 nucleotides, about 2,600 nucleotides to about 5,000 nucleotides, about 2,600 nucleotides to about 4,800 nucleotides, about 2,600 nucleotides to about 4,600 nucleotides, about 2,600 nucleotides to about 4,400 nucleotides, about 2,600 nucleotides to about 4,200 nucleotides, about 2,600 nucleotides to about 4,000 nucleotides, about 2,600 nucleotides to about 3,800 nucleotides, about 2,600 nucleotides to about 3,600 nucleotides, about 2,600 nucleotides to about 3,400 nucleotides, about 2,600 nucleotides to about 3,200 nucleotides, about 2,600 nucleotides to about 3,000 nucleotides, about 2,600 nucleotides to about 2,800 nucleotides, about 2,800 nucleotides to about 10,000 nucleotides, about 2,800 nucleotides to about 9,500 nucleotides, about 2,800 nucleotides to about 9,000 nucleotides, about 2,800 nucleotides to about 8,500 nucleotides, about 2,800 nucleotides to about 8,000 nucleotides, about 2,800 nucleotides to about 7,800 nucleotides, about 2,800 nucleotides to about 7,600 nucleotides, about 2,800 nucleotides to about 7,400 nucleotides, about 2,800 nucleotides to about 7,200 nucleotides, about 2,800 nucleotides to about 7,000 nucleotides, about 2,800 nucleotides to about 6,800 nucleotides, about 2,800 nucleotides to about 6,600 nucleotides, about 2,800 nucleotides to about 6,400 nucleotides, about 2,800 nucleotides to about 6,200 nucleotides, about 2,800 nucleotides to about 6,000 nucleotides, about 2,800 nucleotides to about 5,800 nucleotides, about 2,800 nucleotides to about 5,600 nucleotides, about 2,800 nucleotides to about 5,400 nucleotides, about 2,800 nucleotides to about 5,200 nucleotides, about 2,800 nucleotides to about 5,000 nucleotides, about 2,800 nucleotides to about 4,800 nucleotides, about 2,800 nucleotides to about 4,600 nucleotides, about 2,800 nucleotides to about 4,400 nucleotides, about 2,800 nucleotides to about 4,200 nucleotides, about 2,800 nucleotides to about 4,000 nucleotides, about 2,800 nucleotides to about 3,800 nucleotides, about 2,800 nucleotides to about 3,600 nucleotides, about 2,800 nucleotides to about 3,400 nucleotides, about 2,800 nucleotides to about 3,200 nucleotides, about 2,800 nucleotides to about 3,000 nucleotides, about 3,000 nucleotides to about 10,000 nucleotides, about 3,000 nucleotides to about 9,500 nucleotides, about 3,000 nucleotides to about 9,000 nucleotides, about 3,000 nucleotides to about 8,500 nucleotides, about 3,000 nucleotides to about 8,000 nucleotides, about 3,000 nucleotides to about 7,800 nucleotides, about 3,000 nucleotides to about 7,600 nucleotides, about 3,000 nucleotides to about 7,400 nucleotides, about 3,000 nucleotides to about 7,200 nucleotides, about 3,000 nucleotides to about 7,000 nucleotides, about 3,000 nucleotides to about 6,800 nucleotides, about 3,000 nucleotides to about 6,600 nucleotides, about 3,000 nucleotides to about 6,400 nucleotides, about 3,000 nucleotides to about 6,200 nucleotides, about 3,000 nucleotides to about 6,000 nucleotides, about 3,000 nucleotides to about 5,800 nucleotides, about 3,000 nucleotides to about 5,600 nucleotides, about 3,000 nucleotides to about 5,400 nucleotides, about 3,000 nucleotides to about 5,200 nucleotides, about 3,000 nucleotides to about 5,000 nucleotides, about 3,000 nucleotides to about 4,800 nucleotides, about 3,000 nucleotides to about 4,600 nucleotides, about 3,000 nucleotides to about 4,400 nucleotides, about 3,000 nucleotides to about 4,200 nucleotides, about 3,000 nucleotides to about 4,000 nucleotides, about 3,000 nucleotides to about 3,800 nucleotides, about 3,000 nucleotides to about 3,600 nucleotides, about 3,000 nucleotides to about 3,400 nucleotides, about 3,000 nucleotides to about 3,200 nucleotides, about 3,200 nucleotides to about 10,000 nucleotides, about 3,200 nucleotides to about 9,500 nucleotides, about 3,200 nucleotides to about 9,000 nucleotides, about 3,200 nucleotides to about 8,500 nucleotides, about 3,200 nucleotides to about 8,000 nucleotides, about 3,200 nucleotides to about 7,800 nucleotides, about 3,200 nucleotides to about 7,600 nucleotides, about 3,200 nucleotides to about 7,400 nucleotides, about 3,200 nucleotides to about 7,200 nucleotides, about 3,200 nucleotides to about 7,000 nucleotides, about 3,200 nucleotides to about 6,800 nucleotides, about 3,200 nucleotides to about 6,600 nucleotides, about 3,200 nucleotides to about 6,400 nucleotides, about 3,200 nucleotides to about 6,200 nucleotides, about 3,200 nucleotides to about 6,000 nucleotides, about 3,200 nucleotides to about 5,800 nucleotides, about 3,200 nucleotides to about 5,600 nucleotides, about 3,200 nucleotides to about 5,400 nucleotides, about 3,200 nucleotides to about 5,200 nucleotides, about 3,200 nucleotides to about 5,000 nucleotides, about 3,200 nucleotides to about 4,800 nucleotides, about 3,200 nucleotides to about 4,600 nucleotides, about 3,200 nucleotides to about 4,400 nucleotides, about 3,200 nucleotides to about 4,200 nucleotides, about 3,200 nucleotides to about 4,000 nucleotides, about 3,200 nucleotides to about 3,800 nucleotides, about 3,200 nucleotides to about 3,600 nucleotides, about 3,200 nucleotides to about 3,400 nucleotides, about 3,400 nucleotides to about 10,000 nucleotides, about 3,400 nucleotides to about 9,500 nucleotides, about 3,400 nucleotides to about 9,000 nucleotides, about 3,400 nucleotides to about 8,500 nucleotides, about 3,400 nucleotides to about 8,000 nucleotides, about 3,400 nucleotides to about 7,800 nucleotides, about 3,400 nucleotides to about 7,600 nucleotides, about 3,400 nucleotides to about 7,400 nucleotides, about 3,400 nucleotides to about 7,200 nucleotides, about 3,400 nucleotides to about 7,000 nucleotides, about 3,400 nucleotides to about 6,800 nucleotides, about 3,400 nucleotides to about 6,600 nucleotides, about 3,400 nucleotides to about 6,400 nucleotides, about 3,400 nucleotides to about 6,200 nucleotides, about 3,400 nucleotides to about 6,000 nucleotides, about 3,400 nucleotides to about 5,800 nucleotides, about 3,400 nucleotides to about 5,600 nucleotides, about 3,400 nucleotides to about 5,400 nucleotides, about 3,400 nucleotides to about 5,200 nucleotides, about 3,400 nucleotides to about 5,000 nucleotides, about 3,400 nucleotides to about 4,800 nucleotides, about 3,400 nucleotides to about 4,600 nucleotides, about 3,400 nucleotides to about 4,400 nucleotides, about 3,400 nucleotides to about 4,200 nucleotides, about 3,400 nucleotides to about 4,000 nucleotides, about 3,400 nucleotides to about 3,800 nucleotides, about 3,400 nucleotides to about 3,600 nucleotides, about 3,600 nucleotides to about 10,000 nucleotides, about 3,600 nucleotides to about 9,500 nucleotides, about 3,600 nucleotides to about 9,000 nucleotides, about 3,600 nucleotides to about 8,500 nucleotides, about 3,600 nucleotides to about 8,000 nucleotides, about 3,600 nucleotides to about 7,800 nucleotides, about 3,600 nucleotides to about 7,600 nucleotides, about 3,600 nucleotides to about 7,400 nucleotides, about 3,600 nucleotides to about 7,200 nucleotides, about 3,600 nucleotides to about 7,000 nucleotides, about 3,600 nucleotides to about 6,800 nucleotides, about 3,600 nucleotides to about 6,600 nucleotides, about 3,600 nucleotides to about 6,400 nucleotides, about 3,600 nucleotides to about 6,200 nucleotides, about 3,600 nucleotides to about 6,000 nucleotides, about 3,600 nucleotides to about 5,800 nucleotides, about 3,600 nucleotides to about 5,600 nucleotides, about 3,600 nucleotides to about 5,400 nucleotides, about 3,600 nucleotides to about 5,200 nucleotides, about 3,600 nucleotides to about 5,000 nucleotides, about 3,600 nucleotides to about 4,800 nucleotides, about 3,600 nucleotides to about 4,600 nucleotides, about 3,600 nucleotides to about 4,400 nucleotides, about 3,600 nucleotides to about 4,200 nucleotides, about 3,600 nucleotides to about 4,000 nucleotides, about 3,600 nucleotides to about 3,800 nucleotides, about 3,800 nucleotides to about 10,000 nucleotides, about 3,800 nucleotides to about 9,500 nucleotides, about 3,800 nucleotides to about 9,000 nucleotides, about 3,800 nucleotides to about 8,500 nucleotides, about 3,800 nucleotides to about 8,000 nucleotides, about 3,800 nucleotides to about 7,800 nucleotides, about 3,800 nucleotides to about 7,600 nucleotides, about 3,800 nucleotides to about 7,400 nucleotides, about 3,800 nucleotides to about 7,200 nucleotides, about 3,800 nucleotides to about 7,000 nucleotides, about 3,800 nucleotides to about 6,800 nucleotides, about 3,800 nucleotides to about 6,600 nucleotides, about 3,800 nucleotides to about 6,400 nucleotides, about 3,800 nucleotides to about 6,200 nucleotides, about 3,800 nucleotides to about 6,000 nucleotides, about 3,800 nucleotides to about 5,800 nucleotides, about 3,800 nucleotides to about 5,600 nucleotides, about 3,800 nucleotides to about 5,400 nucleotides, about 3,800 nucleotides to about 5,200 nucleotides, about 3,800 nucleotides to about 5,000 nucleotides, about 3,800 nucleotides to about 4,800 nucleotides, about 3,800 nucleotides to about 4,600 nucleotides, about 3,800 nucleotides to about 4,200 nucleotides, about 3,800 nucleotides to about 4,000 nucleotides, about 4,000 nucleotides to about 10,000 nucleotides, about 4,000 nucleotides to about 9,500 nucleotides, about 4,000 nucleotides to about 9,000 nucleotides, about 4,000 nucleotides to about 8,500 nucleotides, about 4,000 nucleotides to about 8,000 nucleotides, about 4,000 nucleotides to about 7,800 nucleotides, about 4,000 nucleotides to about 7,600 nucleotides, about 4,000 nucleotides to about 7,400 nucleotides, about 4,000 nucleotides to about 7,200 nucleotides, about 4,000 nucleotides to about 7,000 nucleotides, about 4,000 nucleotides to about 6,800 nucleotides, about 4,000 nucleotides to about 6,600 nucleotides, about 4,000 nucleotides to about 6,400 nucleotides, about 4,000 nucleotides to about 6,200 nucleotides, about 4,000 nucleotides to about 6,000 nucleotides, about 4,000 nucleotides to about 5,800 nucleotides, about 4,000 nucleotides to about 5,600 nucleotides, about 4,000 nucleotides to about 5,400 nucleotides, about 4,000 nucleotides to about 5,200 nucleotides, about 4,000 nucleotides to about 5,000 nucleotides, about 4,000 nucleotides to about 4,800 nucleotides, about 4,000 nucleotides to about 4,600 nucleotides, about 4,000 nucleotides to about 4,400 nucleotides, about 4,000 nucleotides to about 4,200 nucleotides, about 4,200 nucleotides to about 10,000 nucleotides, about 4,200 nucleotides to about 9,500 nucleotides, about 4,200 nucleotides to about 9,000 nucleotides, about 4,200 nucleotides to about 8,500 nucleotides, about 4,200 nucleotides to about 8,000 nucleotides, about 4,200 nucleotides to about 7,800 nucleotides, about 4,200 nucleotides to about 7,600 nucleotides, about 4,200 nucleotides to about 7,400 nucleotides, about 4,200 nucleotides to about 7,200 nucleotides, about 4,200 nucleotides to about 7,000 nucleotides, about 4,200 nucleotides to about 6,800 nucleotides, about 4,200 nucleotides to about 6,600 nucleotides, about 4,200 nucleotides to about 6,400 nucleotides, about 4,200 nucleotides to about 6,200 nucleotides, about 4,200 nucleotides to about 6,000 nucleotides, about 4,200 nucleotides to about 5,800 nucleotides, about 4,200 nucleotides to about 5,600 nucleotides, about 4,200 nucleotides to about 5,400 nucleotides, about 4,200 nucleotides to about 5,200 nucleotides, about 4,200 nucleotides to about 5,000 nucleotides, about 4,200 nucleotides to about 4,800 nucleotides, about 4,200 nucleotides to about 4,600 nucleotides, about 4,200 nucleotides to about 4,400 nucleotides, about 4,400 nucleotides to about 10,000 nucleotides, about 4,400 nucleotides to about 9,500 nucleotides, about 4,400 nucleotides to about 9,000 nucleotides, about 4,400 nucleotides to about 8,500 nucleotides, about 4,400 nucleotides to about 8,000 nucleotides, about 4,400 nucleotides to about 7,800 nucleotides, about 4,400 nucleotides to about 7,600 nucleotides, about 4,400 nucleotides to about 7,400 nucleotides, about 4,400 nucleotides to about 7,200 nucleotides, about 4,400 nucleotides to about 7,000 nucleotides, about 4,400 nucleotides to about 6,800 nucleotides, about 4,400 nucleotides to about 6,600 nucleotides, about 4,400 nucleotides to about 6,400 nucleotides, about 4,400 nucleotides to about 6,200 nucleotides, about 4,400 nucleotides to about 6,000 nucleotides, about 4,400 nucleotides to about 5,800 nucleotides, about 4,400 nucleotides to about 5,600 nucleotides, about 4,400 nucleotides to about 5,400 nucleotides, about 4,400 nucleotides to about 5,200 nucleotides, about 4,400 nucleotides to about 5,000 nucleotides, about 4,400 nucleotides to about 4,800 nucleotides, about 4,400 nucleotides to about 4,600 nucleotides, about 4,600 nucleotides to about 10,000 nucleotides, about 4,600 nucleotides to about 9,500 nucleotides, about 4,600 nucleotides to about 9,000 nucleotides, about 4,600 nucleotides to about 8,500 nucleotides, about 4,600 nucleotides to about 8,000 nucleotides, about 4,600 nucleotides to about 7,800 nucleotides, about 4,600 nucleotides to about 7,600 nucleotides, about 4,600 nucleotides to about 7,400 nucleotides, about 4,600 nucleotides to about 7,200 nucleotides, about 4,600 nucleotides to about 7,000 nucleotides, about 4,600 nucleotides to about 6,800 nucleotides, about 4,600 nucleotides to about 6,600 nucleotides, about 4,600 nucleotides to about 6,400 nucleotides, about 4,600 nucleotides to about 6,200 nucleotides, about 4,600 nucleotides to about 6,000 nucleotides, about 4,600 nucleotides to about 5,800 nucleotides, about 4,600 nucleotides to about 5,600 nucleotides, about 4,600 nucleotides to about 5,400 nucleotides, about 4,600 nucleotides to about 5,200 nucleotides, about 4,600 nucleotides to about 5,000 nucleotides, about 4,600 nucleotides to about 4,800 nucleotides, about 4,800 nucleotides to about 10,000 nucleotides, about 4,800 nucleotides to about 9,500 nucleotides, about 4,800 nucleotides to about 9,000 nucleotides, about 4,800 nucleotides to about 8,500 nucleotides, about 4,800 nucleotides to about 8,000 nucleotides, about 4,800 nucleotides to about 7,800 nucleotides, about 4,800 nucleotides to about 7,600 nucleotides, about 4,800 nucleotides to about 7,400 nucleotides, about 4,800 nucleotides to about 7,200 nucleotides, about 4,800 nucleotides to about 7,000 nucleotides, about 4,800 nucleotides to about 6,800 nucleotides, about 4,800 nucleotides to about 6,600 nucleotides, about 4,800 nucleotides to about 6,400 nucleotides, about 4,800 nucleotides to about 6,200 nucleotides, about 4,800 nucleotides to about 6,000 nucleotides, about 4,800 nucleotides to about 5,800 nucleotides, about 4,800 nucleotides to about 5,600 nucleotides, about 4,800 nucleotides to about 5,400 nucleotides, about 4,800 nucleotides to about 5,200 nucleotides, about 4,800 nucleotides to about 5,000 nucleotides, about 5,000 nucleotides to about 10,000 nucleotides, about 5,000 nucleotides to about 9,500 nucleotides, about 5,000 nucleotides to about 9,000 nucleotides, about 5,000 nucleotides to about 8,500 nucleotides, about 5,000 nucleotides to about 8,000 nucleotides, about 5,000 nucleotides to about 7,800 nucleotides, about 5,000 nucleotides to about 7,600 nucleotides, about 5,000 nucleotides to about 7,400 nucleotides, about 5,000 nucleotides to about 7,200 nucleotides, about 5,000 nucleotides to about 7,000 nucleotides, about 5,000 nucleotides to about 6,800 nucleotides, about 5,000 nucleotides to about 6,600 nucleotides, about 5,000 nucleotides to about 6,400 nucleotides, about 5,000 nucleotides to about 6,200 nucleotides, about 5,000 nucleotides to about 6,000 nucleotides, about 5,000 nucleotides to about 5,800 nucleotides, about 5,000 nucleotides to about 5,600 nucleotides, about 5,000 nucleotides to about 5,400 nucleotides, about 5,000 nucleotides to about 5,200 nucleotides, about 5,200 nucleotides to about 10,000 nucleotides, about 5,200 nucleotides to about 9,500 nucleotides, about 5,200 nucleotides to about 9,000 nucleotides, about 5,200 nucleotides to about 8,500 nucleotides, about 5,200 nucleotides to about 8,000 nucleotides, about 5,200 nucleotides to about 7,800 nucleotides, about 5,200 nucleotides to about 7,600 nucleotides, about 5,200 nucleotides to about 7,400 nucleotides, about 5,200 nucleotides to about 7,200 nucleotides, about 5,200 nucleotides to about 7,000 nucleotides, about 5,200 nucleotides to about 6,800 nucleotides, about 5,200 nucleotides to about 6,600 nucleotides, about 5,200 nucleotides to about 6,400 nucleotides, about 5,200 nucleotides to about 6,200 nucleotides, about 5,200 nucleotides to about 6,000 nucleotides, about 5,200 nucleotides to about 5,800 nucleotides, about 5,200 nucleotides to about 5,600 nucleotides, about 5,200 nucleotides to about 5,400 nucleotides, about 5,400 nucleotides to about 10,000 nucleotides, about 5,400 nucleotides to about 9,500 nucleotides, about 5,400 nucleotides to about 9,000 nucleotides, about 5,400 nucleotides to about 8,500 nucleotides, about 5,400 nucleotides to about 8,000 nucleotides, about 5,400 nucleotides to about 7,800 nucleotides, about 5,400 nucleotides to about 7,600 nucleotides, about 5,400 nucleotides to about 7,400 nucleotides, about 5,400 nucleotides to about 7,200 nucleotides, about 5,400 nucleotides to about 7,000 nucleotides, about 5,400 nucleotides to about 6,800 nucleotides, about 5,400 nucleotides to about 6,600 nucleotides, about 5,400 nucleotides to about 6,400 nucleotides, about 5,400 nucleotides to about 6,200 nucleotides, about 5,400 nucleotides to about 6,000 nucleotides, about 5,400 nucleotides to about 5,800 nucleotides, about 5,400 nucleotides to about 5,600 nucleotides, about 5,600 nucleotides to about 10,000 nucleotides, about 5,600 nucleotides to about 9,500 nucleotides, about 5,600 nucleotides to about 9,000 nucleotides, about 5,600 nucleotides to about 8,500 nucleotides, about 5,600 nucleotides to about 8,000 nucleotides, about 5,600 nucleotides to about 7,800 nucleotides, about 5,600 nucleotides to about 7,600 nucleotides, about 5,600 nucleotides to about 7,400 nucleotides, about 5,600 nucleotides to about 7,200 nucleotides, about 5,600 nucleotides to about 7,000 nucleotides, about 5,600 nucleotides to about 6,800 nucleotides, about 5,600 nucleotides to about 6,600 nucleotides, about 5,600 nucleotides to about 6,400 nucleotides, about 5,600 nucleotides to about 6,200 nucleotides, about 5,600 nucleotides to about 6,000 nucleotides, about 5,600 nucleotides to about 5,800 nucleotides, about 5,800 nucleotides to about 10,000 nucleotides, about 5,800 nucleotides to about 9,500 nucleotides, about 5,800 nucleotides to about 9,000 nucleotides, about 5,800 nucleotides to about 8,500 nucleotides, about 5,800 nucleotides to about 8,000 nucleotides, about 5,800 nucleotides to about 7,800 nucleotides, about 5,800 nucleotides to about 7,600 nucleotides, about 5,800 nucleotides to about 7,400 nucleotides, about 5,800 nucleotides to about 7,200 nucleotides, about 5,800 nucleotides to about 7,000 nucleotides, about 5,800 nucleotides to about 6,800 nucleotides, about 5,800 nucleotides to about 6,600 nucleotides, about 5,800 nucleotides to about 6,400 nucleotides, about 5,800 nucleotides to about 6,200 nucleotides, about 5,800 nucleotides to about 6,000 nucleotides, about 6,000 nucleotides to about 10,000 nucleotides, about 6,000 nucleotides to about 9,500 nucleotides, about 6,000 nucleotides to about 9,000 nucleotides, about 6,000 nucleotides to about 8,500 nucleotides, about 6,000 nucleotides to about 8,000 nucleotides, about 6,000 nucleotides to about 7,800 nucleotides, about 6,000 nucleotides to about 7,600 nucleotides, about 6,000 nucleotides to about 7,400 nucleotides, about 6,000 nucleotides to about 7,200 nucleotides, about 6,000 nucleotides to about 7,000 nucleotides, about 6,000 nucleotides to about 6,800 nucleotides, about 6,000 nucleotides to about 6,600 nucleotides, about 6,000 nucleotides to about 6,400 nucleotides, about 6,000 nucleotides to about 6,200 nucleotides, about 6,200 nucleotides to about 10,000 nucleotides, about 6,200 nucleotides to about 9,000 nucleotides, about 6,200 nucleotides to about 8,500 nucleotides, about 6,200 nucleotides to about 8,000 nucleotides, about 6,200 nucleotides to about 7,800 nucleotides, about 6,200 nucleotides to about 7,600 nucleotides, about 6,200 nucleotides to about 7,400 nucleotides, about 6,200 nucleotides to about 7,200 nucleotides, about 6,200 nucleotides to about 7,000 nucleotides, about 6,200 nucleotides to about 6,800 nucleotides, about 6,200 nucleotides to about 6,600 nucleotides, about 6,200 nucleotides to about 6,400 nucleotides, about 6,400 nucleotides to about 10,000 nucleotides, about 6,400 nucleotides to about 9,500 nucleotides, about 6,400 nucleotides to about 9,000 nucleotides, about 6,400 nucleotides to about 8,500 nucleotides, about 6,400 nucleotides to about 8,000 nucleotides, about 6,400 nucleotides to about 7,800 nucleotides, about 6,400 nucleotides to about 7,600 nucleotides, about 6,400 nucleotides to about 7,400 nucleotides, about 6,400 nucleotides to about 7,200 nucleotides, about 6,400 nucleotides to about 7,000 nucleotides, about 6,400 nucleotides to about 6,800 nucleotides, about 6,400 nucleotides to about 6,600 nucleotides, about 6,600 nucleotides to about 10,000 nucleotides, about 6,600 nucleotides to about 9,500 nucleotides, about 6,600 nucleotides to about 9,000 nucleotides, about 6,600 nucleotides to about 8,500 nucleotides, about 6,600 nucleotides to about 8,000 nucleotides, about 6,600 nucleotides to about 7,800 nucleotides, about 6,600 nucleotides to about 7,600 nucleotides, about 6,600 nucleotides to about 7,400 nucleotides, about 6,600 nucleotides to about 7,200 nucleotides, about 6,600 nucleotides to about 7,000 nucleotides, about 6,600 nucleotides to about 6,800 nucleotides, about 6,800 nucleotides to about 10,000 nucleotides, about 6,800 nucleotides to about 9,500 nucleotides, about 6,800 nucleotides to about 9,000 nucleotides, about 6,800 nucleotides to about 8,500 nucleotides, about 6,800 nucleotides to about 8,000 nucleotides, about 6,800 nucleotides to about 7,800 nucleotides, about 6,800 nucleotides to about 7,600 nucleotides, about 6,800 nucleotides to about 7,400 nucleotides, about 6,800 nucleotides to about 7,200 nucleotides, about 6,800 nucleotides to about 7,000 nucleotides, about 7,000 nucleotides to about 10,000 nucleotides, about 7,000 nucleotides to about 9,500 nucleotides, about 7,000 nucleotides to about 9,000 nucleotides, about 7,000 nucleotides to about 8,500 nucleotides, about 7,000 nucleotides to about 8,000 nucleotides, about 7,000 nucleotides to about 7,800 nucleotides, about 7,000 nucleotides to about 7,600 nucleotides, about 7,000 nucleotides to about 7,400 nucleotides, about 7,000 nucleotides to about 7,200 nucleotides, about 7,200 nucleotides to about 10,000 nucleotides, about 7,200 nucleotides to about 9,500 nucleotides, about 7,200 nucleotides to about 9,000 nucleotides, about 7,200 nucleotides to about 8,500 nucleotides, about 7,200 nucleotides to about 8,000 nucleotides, about 7,200 nucleotides to about 7,800 nucleotides, about 7,200 nucleotides to about 7,600 nucleotides, about 7,200 nucleotides to about 7,400 nucleotides, about 7,400 nucleotides to about 10,000 nucleotides, about 7,400 nucleotides to about 9,500 nucleotides, about 7,400 nucleotides to about 9,000 nucleotides, about 7,400 nucleotides to about 8,500 nucleotides, about 7,400 nucleotides to about 8,000 nucleotides, about 7,400 nucleotides to about 7,800 nucleotides, about 7,400 nucleotides to about 7,600 nucleotides, about 7,600 nucleotides to about 10,000 nucleotides, about 7,600 nucleotides to about 9,500 nucleotides, about 7,600 nucleotides to about 9,000 nucleotides, about 7,600 nucleotides to about 8,500 nucleotides, about 7,600 nucleotides to about 8,000 nucleotides, about 7,600 nucleotides to about 7,800 nucleotides, about 7,800 nucleotides to about 10,000 nucleotides, about 7,800 nucleotides to about 9,500 nucleotides, about 7,800 nucleotides to about 9,000 nucleotides, about 7,800 nucleotides to about 8,500 nucleotides, about 7,800 nucleotides to about 8,000 nucleotides, about 8,000 nucleotides to about 10,000 nucleotides, about 8,000 nucleotides to about 9,500 nucleotides, about 8,000 nucleotides to about 9,000 nucleotides, about 8,000 nucleotides to about 8,500 nucleotides, about 8,500 nucleotides to about 10,000 nucleotides, about 8,500 nucleotides to about 9,500 nucleotides, about 8,500 nucleotides to about 9,000 nucleotides, about 9,000 nucleotides to about 10,000 nucleotides, about 9,000 nucleotides to about 9,500 nucleotides, or about 9,500 nucleotides to about 10,000 nucleotides (inclusive).

A variety of different methods known in the art can be used to introduce any of vectors disclosed herein into a primate cell (e.g., a supporting cochlear outer hair cell or a hair cell (e.g., an inner hair cell or an outer hair cell). Non-limiting examples of methods for introducing nucleic acid into a primate cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the vectors described herein can be introduced into a primate cell by, for example, lipofection.

Various molecular biology techniques that can be used to introduce a mutation(s) and/or a deletion(s) into an endogenous gene are also known in the art. Non-limiting examples of such techniques include site-directed mutagenesis, CRISPR (e.g., CRISPR/Cas9-induced knock-in mutations and CRISPR/Cas9-induced knock-out mutations), TALENs, and base editing (Rees & Lui, Nature Rev. Genetics 19: 770-788, 2018; Eid et al., Biochem J. 475(11): 1955-1964, 2018). These methods can be used to correct the sequence of a defective endogenous gene present in a chromosome of a target cell.

Any of the vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, and a Kozak consensus sequence. Non-limiting examples of these control sequences are described herein. In some embodiments, a promoter can be a native promoter, a constitutive promoter, an inducible promoter, and/or a tissue-specific promoter.

Some embodiments of any of the compositions and kits described herein can include any combination of the AAV vectors described herein. Some embodiments of any of the methods described herein can include the use of any combination of the AAV vectors described herein.

### Promoters

The term “promoter” means a DNA sequence recognized by enzymes/proteins in a primate cell required to initiate the transcription of a specific gene (e.g., a supporting cell target gene or a hair cell target gene). A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, a vector encoding an N-terminal portion of a hair cell target protein (e.g., a human hair cell target protein) or a hair cell target protein can include a promoter and/or an enhancer. The vector encoding the N-terminal portion of the hair cell target protein or a hair cell target protein can include any of the promoters and/or enhancers described herein or known in the art.

In some embodiments, a vector encoding an N-terminal portion of a supporting cell target protein (e.g., a human supporting cell target protein) or a supporting cell target protein can include a promoter and/or an enhancer. The vector encoding the N-terminal portion of the supporting cell target protein or a supporting cell target protein can include any of the promoters and/or enhancers described herein or known in the art.

In some embodiments, the promoter is an inducible promoter, a constitutive promoter, a primate cell promoter, a viral promoter, a chimeric promoter, an engineered promoter, a tissue-specific promoter, or any other type of promoter known in the art. In some embodiments, the promoter is a RNA polymerase II promoter, such as a primate RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in cochlear cells such as hair cells. In some examples, the promoter is a cochlea-specific promoter or a cochlea-oriented promoter.

A variety of promoters are known in the art that can be used herein. Non-limiting examples of promoters that can be used herein include: human EF1a, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CAG promoter or a CAG/CBA promoter.

The term “constitutive” promoter refers to a nucleotide sequence that, when operably linked with a nucleic acid encoding a target protein (e.g., a supporting cell target protein, a hair cell target protein), causes RNA to be transcribed from the nucleic acid in a primate cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory proteins that bind to the tissue-specific promoter).

In some embodiments, the regulatory sequences impart tissue-specific gene expression capabilities. In some cases, the tissue-specific regulatory sequences bind tissue-specific transcription factors that induce transcription in a tissue-specific manner.

Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a primate desmin (DES) promoter, an alpha-myosin heavy chain (a-MHC) promoter, and a cardiac Troponin T (cTnT) promoter. Additional exemplary promoters include Beta-actin promoter, hepatitis B virus core promoter (Sandig et al., Gene Ther. 3:1002-1009, 1996), alpha-fetoprotein (AFP) promoter (Arbuthnot et al., Hum. Gene Ther. 7:1503-1514, 1996), bone osteocalcin promoter (Stein et al., Mol. Biol. Rep. 24:185-196, 1997); bone sialoprotein promoter (Chen et al., J. Bone Miner. Res. 11:654-664, 1996), CD2 promoter (Hansal et al., J. Immunol. 161:1063-1068, 1998); immunoglobulin heavy chain promoter; T cell receptor alpha-chain promoter, neuronal such as neuron-specific enolase (NSE) promoter (Andersen et al., Cell. Mol. Neurobiol. 13:503-515, 1993), neurofilament light-chain gene promoter (Piccioli et al., Proc. Natl. Acad. Sci. U.S.A. 88:5611-5615, 1991), and the neuron-specific vgf gene promoter (Piccioli et al., Neuron 15:373-384, 1995).

In some embodiments, the tissue-specific promoter is a cochlea-specific promoter. In some embodiments, the tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter. In some embodiments, the promoter is an outer hair cell-specific promoter such as a PRESTIN promoter or an ONCOMOD promoter. See, e.g., Zheng et al., Nature 405:149-155, 2000; Tian et al. Dev. Dyn. 231:199-203, 2004; and Ryan et al., Adv. Otorhinolaryngol. 66: 99-115, 2009.

In some embodiments of any of the AAV vectors described herein, the AAV vector includes a human ATOH1 enhancer-promoter (SEQ ID NO: 84).

Human ATOH1 enhancer-promoter
(SEQ ID NO: 84)
ctatggagtttgcataacaaacgtttggcagctcgctctottacactccattaacaagctgtaacatatag
ctgcaggttgctataatctcattaatattttggaaacttgaatattgagtatttctgagtgctcattcccc
atatgccagccacttctgccatgctgactggttcctttctctccattattagcaattagcttcttaccttc
caaagtcagatccaaggtatccaagatactagcaaaggaatcaactatgtgtgcaagttaagcatgcttaa
tatcacccaaacaaacaaagaggcagcatttcttaaagtaatgaagatagataaatcgggttagtcctttg
cgacactgctggtgctttctagagttttatatattttaagcagcttgctttatattctgtctttgcctccc
accccaccagcacttttatttgtggagggttttggctcgccacactttgggaaacttatttgatttcacgg
agagctgaaggaagatcatttttggcaacagacaagtttaaacacgatttctatgggacattgctaactgg
ggcccctaaggagaaaggggaaactgagcggagaatgggttaaatccttggaagcaggggagaggcagggg
aggagagaagtcggaggagtataaagaaaaggacaggaaccaagaagcgtgggggtggtttgccgtaatgt
gagtgtttcttaattagagaacggttgacaatagagggtctggcagaggctcctggccgcggtgcggagcg
tctggagcggagcacgcgctgtcagctggtgagcgcactctcctttcaggcagctccccggggagctgtgc
ggccacatttaacaccatcatcacccctccccggcctcctcaacctcggcctcctcctcgtcgacagcctt
ccttggcccccaccagcagagctcacagtagcgagcgtctctcgccgtctcccgcactcggccggggcctc
tctcctcccccagctgcgcagcgggagccgccactgcccactgcacctcccagcaaccagcccagcacgca
aagaagctgcgcaaagttaaagccaagcaatgccaaggggaggggaagctggaggcgggctttgagtggct
tctgggcgcctggcgggtccagaatcgcccagagccgcccgcggtcgtgcacatctgacccgagtcagctt
gggcaccagccgagagccggctccgcaccgctcccgcaccccagccgccggggtggtgacacacaccggag
tcgaattacagccctgcaattaacatatgaatctgacgaatttaaaagaaggaaaaaaaaaaaaaaacctg
agcaggcttgggagtcctctgcacacaagaacttttctcggggtgtaaaaactctttgattggctgctcgc
acgcgcctgcccgcgccctccattggctgagaagacacgcgaccggcgcgaggagggggttgggagaggag
cggggggagactgagtggcgcgtgccgctttttaaaggggcgcagcgccttcagcaaccggagaagcatag
ttgcacgcgacctggtgtgtgatctccgagtgggtgggggagggtcgaggagggaaaaaaaaataagacgt
tgcagaagagacccggaaagggccttttttttggttgagctggtgtcccagtgctgcctccgatcctgagc
ctccgagcctttgcagtgcaa

### Enhancers and 5′ Cap

In some instances, a vector can include a promoter sequence and/or an enhancer sequence. The term “enhancer” refers to a nucleotide sequence that can increase the level of transcription of a nucleic acid encoding a protein of interest (e.g., a supporting cell target protein or a hair cell target protein) Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer.

### Destabilizing Domain (DD)

Any of the AAV vectors provided herein can optionally include a sequence encoding a destabilizing domain (“a destabilizing sequence”) for temporal control of protein expression. Non-limiting examples of destabilizing sequences include sequences encoding: a FK506 sequence, a dihydrofolate reductase (DHFR) sequence.

Exemplary DHFR destabilizing sequence
(SEQ ID NO: 85):
MISLIAALAVDYVIGMENAMPWNLPADLAWFKRNTLNKPVIMGRHTWESIGRPLPGRKNIILSSQPSTDDR
VTWVKSVDEAIAACGDVPEIMVIGGGRVIEQFLPKAQKLYLTHIDAEVEGDTHFPDYEPDDWESVESEFHD
ADAQNSHSYCFEILERR
Exemplary DHFR destabilizing domain sequence
(SEQ ID NO: 86)
ggtaccatcagtotgattqcggcgttagoggtagattacgttatoggcatggaaaacgccatgccgtggaa
cctqcctqccgatctcgcctggtttaaacgcaacaccttaaataaacccgtgattatgggccgccatacct
gggaatcaatoggtcgtccgttgccaggacgcaaaaatattatcctcagcagtcaaccgagtacggacgat
cgcgtaacgtgggtgaagtoggtggatgaagccatcgcggcgtgtggtgacgtaccagaaatcatggtgat
tggcggcggtcgcgttattgaacagttottgccaaaagcgcaaaaactqtatctgacgcatatcgacgcag
aagtggaaggcgacacccatttccoggattacgagccggatgactgggaatcggtattcagcgaattccac
gatqctgatqcgcagaactotcacagctattgotttgagattctggagoggcgataa

In some embodiments of any of the AAV vectors described herein, the AAV vector includes a destabilizing domain (SEQ ID NO: 87).

Exemplary destabilizing domain
(SEQ ID NO: 87)
atcagtctgattgoggcgttagcggtagattacgttatcggcatggaaaacgccatgccgtggaacctgcc
tgccgatctcgcctggtttaaacgcaacaccttaaataaacccgtgattatgggccgccatacctgggaat
caatcggtcgtccgttgccaggacgcaaaaatattatcctcagcagtcaaccgagtacggacgatcgcgta
acgtgggtgaagtcggtggatgaagccatcgcggcgtgtggtgacgtaccagaaatcatggtgattggcgg
cggtcgcgttattgaacagttcttgccaaaagcgcaaaaactgtatctgacgcatatcgacgcagaagtgg
aaggcgacacccatttcccggattacgagccggatgactgggaatcggtattcagcgaattccacgatgct
gatgcgcagaactctcacagctattgctttgagattctggagcggcga

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al. (2012) Cell 126(5): 995-1004).

Exemplary FKBP12 destabilizing sequence
(SEQ ID NO: 88)
MGVEKQVIRPGNGPKPAPGQTVTVHCTGEGKDGDLSQKFWSTKDEGQKPFSFQIGKGAVIKGWDEGVIGMQ
IGEVARLRCSSDYAYGAGGFPAWGIQPNSVLDFEIEVLSVQ

In some embodiments, the destabilizing sequence is a DHFR sequence, and the stabilizing ligand is trimethoprim (TMP) (Iwamoto et al. (2010) Chem Biol 17:981-988).

In the absence of a stabilizing ligand, the protein sequence operatively linked to the destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the destabilizing sequence is a FKBP12 sequence, and the presence of an AAV vector carrying the FKBP12 gene in a primate cell (e.g., a supporting cochlear outer hair cell) is detected by western blotting. In some embodiments, the destabilizing sequence can be used to verify the temporally-specific activity of any of the AAV vectors described herein.

### Poly(A) Sequences

In some embodiments, any of the vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) sequence is positioned 3′ to the nucleic acid sequence encoding the C-terminus of the hair cell target protein. In some embodiments, the poly(A) sequence is positioned 3′ to the nucleic acid sequence encoding the C-terminus of the supporting cell target protein or a protein of interest (e.g., a Cas9 endonuclease, e.g., a SaCas9 endonuclease (e.g., any of the SaCas9 endonucleases described herein), a reporter protein (e.g., a GFP protein, a mScarlet protein)).

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation signal or “poly(A) sequence.” The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) sequence” is a sequence that triggers the endonuclease cleavage of an mRNA and the additional of a series of adenosines to the 3′ end of the cleaved mRNA.

There are several poly(A) sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7):1340-1347, 2007), the group of SV40 poly(A) sites, such as the SV40 late and early poly(A) site (Schek et al., Mol. Cell Biol. 12(12):5386-5393, 1992).

The poly(A) sequence can be a sequence of AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) sequence can be a synthetic polyadenylation site (see, e.g., the pCl-neo expression vector of Promega which is based on Levitt el al, Genes Dev. 3(7):1019-1025, 1989). In some embodiments, the poly(A) sequence is the polyadenylation signal of soluble neuropilin-1 (sNRP) (AAATAAAATACGAAATG, SEQ ID NO: 90) (see, e.g., WO 05/073384). Additional examples of poly(A) sequences are known in the art.

### Internal Ribosome Entry Site (IRES)

In some embodiments, a vector encoding the C-terminus of the hair cell target protein or a hair cell target protein can include a polynucleotide internal ribosome entry site (IRES). In some embodiments, a vector encoding the C-terminus of the supporting cell target protein or a supporting cell target protein can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (11W), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the vector that encodes the C-terminus of a hair cell target protein or a hair cell target protein is the foot and mouth disease virus (FMDV). In some embodiments, the IRES sequence that is incorporated into the vector that encodes the C-terminus of a supporting cell target protein or a supporting cell target protein is the foot and mouth disease virus (FMDV) 2A sequence. In some embodiments, the IRES sequence that is incorporated into the vector that encodes the C-terminal portion of a protein of interest (e.g., a Cas9 endonuclease, e.g., a SaCas9 endonuclease (e.g., any of the SaCas9 endonucleases described herein)) is the FMDV 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

### Reporter Sequences/Detectable Marker Genes

Any of the vectors provided herein can optionally include a sequence encoding a reporter protein or a detectable marker (“a reporter sequence” or “detectable marker gene”). Non-limiting examples of reporter sequences or detectable marker genes include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences and detectable marker genes are known in the art. When associated with regulatory elements which drive their expression, the reporter sequence or detectable marker gene can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence or detectable marker gene is the LacZ gene, and the presence of a vector carrying the LacZ gene in a primate cell (e.g., a supporting cochlear outer hair cell or a hair cell (e.g., a cochlear outer hair cell or a cochlear inner hair cell)) is detected by assays for beta-galactosidase activity. In other embodiments, the reporter or detectable marker is a fluorescent protein (e.g., green fluorescent protein) or luciferase, the presence of a vector carrying the fluorescent protein or luciferase in a primate cell (e.g., a supporting cochlear outer hair cell or a hair cell (e.g., a cochlear outer hair cell, a cochlear inner hair cell)) may be measured by fluorescent techniques (e.g., fluorescent microscopy or FACS) or light production in a luminometer (e.g., a spectrophotometer or an IVIS imaging instrument). In some embodiments, the reporter sequence or detectable marker can be used to verify the tissue-specific targeting capabilities and tissue-specific promoter regulatory activity of any of the vectors described herein.

### Flanking Regions Untranslated Regions (UTRs)

In some embodiments, any of the vectors described herein (e.g., any of the at least two different vectors) can include an untranslated region. In some embodiments, a vector can includes a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the stability of a supporting cell target protein or a hair cell target protein or of a protein of interest (e.g., a Cas9 endonuclease, e.g., a SaCas9 endonuclease (e.g., any of the SaCas9 endonucleases described herein), a reporter protein (e.g., a GFP protein, a mScarlet protein).

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), which is followed by another “G”. The 5′ UTR have also been known, e.g., to form secondary structures that are involved in elongation factor binding.

For example, in some embodiments, a 5′ UTR is included in any of the vectors described herein. Non-limiting examples of 5′ UTRs including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the cochlea can be included in any of the vectors, compositions, kits, and methods described herein.

3′ UTRs are known to have stretches of adenosines and uridines embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a hair cell target protein. In some embodiments, the introduction, removal, or modification of 3′ UTR

AREs can be used to modulate the stability of an mRNA encoding a supporting cell target protein. In other embodiments, AREs can be removed or mutated to increase the intracellular stability and thus increase translation and production of a hair cell target protein. In other embodiments, AREs can be removed or mutated to increase the intracellular stability and thus increase translation and production of a supporting cell target protein.

In other embodiments, non-UTR sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels.

### Primate Cells

Also provided herein is a cell (e.g., a primate cell) that includes any of the nucleic acids, vectors (e.g., at least two different vectors described herein), or compositions described herein. In some embodiments, the primate cell is a human cell (e.g., a human supporting cell or a human hair cell). In other embodiments, the primate is a non-human primate (e.g., simian cell (e.g., a monkey cell (e.g., a marmoset cell, a baboon cell, a macaque cell), or an ape cell (e.g., a gorilla cell, a gibbon cell, an orangutan cell, a chimpanzee cell). Skilled practitioners will appreciate that the nucleic acids and vectors described herein can be introduced into any primate cell (e.g., a primate supporting cell or a primate hair cell). Non-limiting examples of vectors and methods for introducing vectors into primate cells are described herein.

In some embodiments, the primate cell can be a supporting hair cell of the inner ear of a mammal. For example, a supporting cell can be Hensen's cells, Deiters' cells, inner pillar cells, outer pillar cells, Claudius cells, inner border cells, inner phalangeal cells, or cells of the stria vascularis.

In some embodiments, the primate cell is a specialized cell of the cochlea. In some embodiments, the primate cell is a hair cell. In some embodiments, the primate cell is a cochlear inner hair cell or a cochlear outer hair cell. In some embodiments, the primate cell is a cochlear inner hair cell. In some embodiments, the primate cell is a cochlear outer hair cell.

In some embodiments, the primate cell is in vitro. In some embodiments, the primate cell is present in a primate. In some embodiments, the primate cell is autologous cell obtained from a primate and cultured ex vivo.

### Methods

Also provided herein are methods of correcting a hair cell target gene defect in a hair cell of an inner ear in a primate that include: administering to the inner ear of the primate a therapeutically effective amount of any of the compositions described herein, where the administering repairs the hair cell target gene defect in the hair cell of the inner ear of the primate.

Also provided herein are methods of increasing the expression level of a hair cell target protein in a hair cell of an inner ear of a primate that include: administering to the inner ear of the primate a therapeutically effective amount of any of the compositions described herein, where the administering results in an increase in the expression level of the hair cell target protein in the hair cell of the inner ear of the primate.

Also provided herein are methods of treating hearing loss (e.g., non-syndromic sensorineural hearing loss or syndromic sensorineural hearing loss) in a primate identified as having a defective hair cell target gene that include: administering to the inner ear of the primate a therapeutically effective amount of any of the compositions described herein.

In some embodiments of any of these methods, the primate has been previously identified as having a defective hair cell target gene (e.g., a hair cell target gene having a mutation that results in a decrease in the expression and/or activity of a hair cell target protein encoded by the gene). Some embodiments of any of these methods further include, prior to the introducing or administering step, determining that the primate has a defective hair cell target gene. Some embodiments of any of these methods can further include detecting a mutation in a hair cell target gene in a primate. Some embodiments of any of the methods can further include identifying or diagnosing a primate as having non-syndromic sensorineural hearing loss. Some embodiments of any of the methods can further include identifying or diagnosing a primate as having syndromic sensorineural hearing loss.

Also provided herein are methods of correcting a supporting cell target gene defect in a supporting cell of an inner ear in a primate that include: administering to the inner ear of the primate a therapeutically effective amount of any of the compositions described herein, where the administering repairs the supporting cell target gene defect in the supporting cell of the inner ear of the primate.

Also provided herein are methods of increasing the expression level of a supporting cell target protein in a supporting cell of an inner ear of a primate that include: administering to the inner ear of the primate a therapeutically effective amount of any of the compositions described herein, where the administering results in an increase in the expression level of the supporting cell target protein in the supporting cell of the inner ear of the primate.

Also provided herein are methods of treating hearing loss (e.g., non-syndromic sensorineural hearing loss or syndromic sensorineural hearing loss) in a primate identified as having a defective supporting cell target gene that include: administering to the inner ear of the primate a therapeutically effective amount of any of the compositions described herein.

Also provided herein are methods of restoring hearing in a primate identified or diagnosed as having hearing loss that include: administering to the inner ear of the primate a therapeutically effective amount of any compositions described herein.

Also provided herein are methods of restoring synapses and/or preserving spiral ganglion nerves in a primate identified or diagnosed as having an inner ear disorder that include: administering to the inner ear of the primate a therapeutically effective amount of any of the compositions described herein.

Also provided herein are methods that include administering to an inner ear of a primate a therapeutically effective amount of any of the compositions described herein.

In some embodiments of any of these methods, the primate has been previously identified as having a defective supporting cell target gene (e.g., a supporting cell target gene having a mutation that results in a decrease in the expression and/or activity of a supporting cell target protein encoded by the gene). Some embodiments of any of these methods further include, prior to the introducing or administering step, determining that the primate has a defective supporting cell target gene. Some embodiments of any of these methods can further include detecting a mutation in a supporting cell target gene in a primate. Some embodiments of any of the methods can further include identifying or diagnosing a primate as having non-syndromic sensorineural hearing loss. Some embodiments of any of the methods can further include identifying or diagnosing a primate as having syndromic sensorineural hearing loss.

In some embodiments of any of these methods, two or more doses of any of the compositions described herein are introduced or administered into the cochlea of the primate. Some embodiments of any of these methods can include introducing or administering a first dose of the composition into the cochlea of the primate, assessing hearing function of the primate following the introducing or the administering of the first dose, and administering an additional dose of the composition into the cochlea of the primate found not to have a hearing function within a normal range (e.g., as determined using any test for hearing known in the art).

In some embodiments of any of the methods described herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any of the methods described herein, the primate has or is at risk of developing non-syndromic sensorineural hearing loss. In some embodiments of any of the methods described herein, the primate has been previously identified as having a mutation in a hair cell target gene. In some embodiments of any of the methods described herein, the primate has been previously identified as having a mutation in a supporting cell target gene. In some embodiments of any of the methods described herein, the primate has any of the mutations in a hair cell target gene that are described herein or are known in the art to be associated with non-syndromic sensorineural hearing loss or syndromic sensorineural hearing loss. In some embodiments of any of the methods described herein, the primate has any of the mutations in a supporting cell target gene that are described herein or are known in the art to be associated with non-syndromic sensorineural hearing loss or syndromic sensorineural hearing loss.

In some embodiments of any of the methods described herein, the primate has been identified as being a carrier of a mutation in a hair cell target gene (e.g., via genetic testing). In some embodiments of any of the methods described herein, the primate has been identified as being a carrier of a mutation in a supporting cell target gene (e.g., via genetic testing). In some embodiments of any of the methods described herein, the primate has been identified as having a mutation in a hair cell target gene and has been diagnosed with hearing loss (e.g., non-syndromic sensorineural hearing loss or syndromic sensorineural hearing loss). In some embodiments of any of the methods described herein, the primate has been identified as having a mutation in a supporting cell target gene and has been diagnosed with hearing loss (e.g., non-syndromic sensorineural hearing loss or syndromic sensorineural hearing loss). In some embodiments of any of the methods described herein, the primate has been identified as having hearing loss (e.g., non-syndromic sensorineural hearing loss or syndromic sensorineural hearing loss). In some embodiments, successful treatment of hearing loss (e.g., non-syndromic sensorineural hearing loss or syndromic sensorineural hearing loss) can be determined in a primate using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments of these methods, the primate cell is in vitro. In some embodiments of these methods, the primate cell is originally obtained from a primate and is cultured ex vivo. In some embodiments, the primate cell has previously been determined to have a defective hair cell target gene. In some embodiments, the primate cell has previously been determined to have a defective supporting cell target gene.

Methods for introducing any of the compositions described herein into a primate cell are known in the art (e.g., via lipofection or through the use of a viral vector, e.g., any of the viral vectors described herein).

An increase in expression of an active target protein (e.g., an active supporting cell target protein (e.g., a full-length supporting cell target protein) or an active hair cell target protein (e.g., a full-length hair cell target protein) as described herein is, e.g., as compared to a control or to the level of expression of an active target protein (e.g., an active supporting cell target protein (e.g., a full-length supporting cell target protein) or an active hair cell target protein (e.g., a full-length hair cell target protein) prior to the introduction of the vector(s).

Methods of detecting expression and/or activity of a target protein (e.g., a supporting cell target protein or a hair cell target protein) are known in the art. In some embodiments, the level of expression of a hair cell target protein can be detected directly (e.g., detecting hair cell target protein or detecting hair cell target mRNA). In some embodiments, the level of expression of a supporting cell target protein can be detected directly (e.g., detecting supporting cell target protein or detecting supporting cell target mRNA). Non-limiting examples of techniques that can be used to detect expression and/or activity of a target protein (e.g., a supporting cell target protein or a hair cell target protein) directly include: real-time PCR, Western blotting, immunoprecipitation, immunohistochemistry, or immunofluorescence. In some embodiments, expression of a hair cell target protein can be detected indirectly (e.g., through functional hearing tests). In some embodiments, expression of a supporting cell target protein can be detected indirectly (e.g., through functional hearing tests).

### Pharmaceutical Compositions and Kits

In some embodiments, any of the compositions described herein can further include one or more agents that promote the entry of a nucleic acid or any of the vectors described herein into a primate cell (e.g., a liposome or cationic lipid).

In some embodiments, any of the vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRUDOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a primate cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

Any of the compositions described herein can be, e.g., a pharmaceutical composition. A pharmaceutical composition can include any of the compositions described herein and one or more pharmaceutically or physiologically acceptable carriers, diluents, or excipients. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein.

In some embodiments, a single dose of any of the compositions described herein can include a total sum amount of the at least two different vectors of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 μg, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 μg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions provided herein can be, e.g., formulated to be compatible with their intended route of administration. A non-limiting example of an intended route of administration is local administration (e.g., intra-cochlear administration). In some embodiments, the therapeutic compositions are formulated to include a lipid nanoparticle. In some embodiments, the therapeutic compositions are formulated to include a polymeric nanoparticle. In some embodiments, the therapeutic compositions are formulated to include a mini-circle DNA. In some embodiments, the therapeutic compositions are formulated to comprise a CELiD DNA. In some embodiments, the therapeutic compositions are formulated to comprise a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the compositions described herein. In some embodiments, a kit can include a solid composition (e.g., a lyophilized composition including the at least two different vectors described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Devices and Surgical Methods

Provided herein are therapeutic delivery systems for treating hearing loss (e.g., non-syndromic sensorineural hearing loss or syndromic sensorineural hearing loss). In one aspect, the therapeutic delivery systems include i) a medical device capable of creating one or a plurality of incisions in a round window membrane of an inner ear of a primate in need thereof, and ii) an effective dose of a composition (e.g., any of the compositions described herein). In some embodiments, the medical device includes a plurality of micro-needles.

Also provided herein are surgical methods for treatment of hearing loss (e.g., non-syndromic sensorineural hearing loss or syndromic sensorineural hearing loss). In some embodiments, the methods include the steps of: introducing into a cochlea of a primate a first incision at a first incision point; and administering intra-cochlearly a therapeutically effective amount of any of the compositions provided herein. In some embodiments, the composition is administered to the primate at the first incision point. In some embodiments, the composition is administered to the primate into or through the first incision.

In some embodiments of any of the methods described herein, any of the compositions described herein is administered to the primate into or through the cochlea oval window membrane. In some embodiments of any of the methods described herein, any of the compositions described herein is administered to the primate into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations that become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### EXAMPLES

### Example 1. AAV Single Vector Injection into the Inner Ear

Immunofluorescent staining was performed on cochlear tissue of a cynomolgus macaque (non-human primate) following administration of a single Anc80-GFP AAV vector directly into the inner ear through the round window.

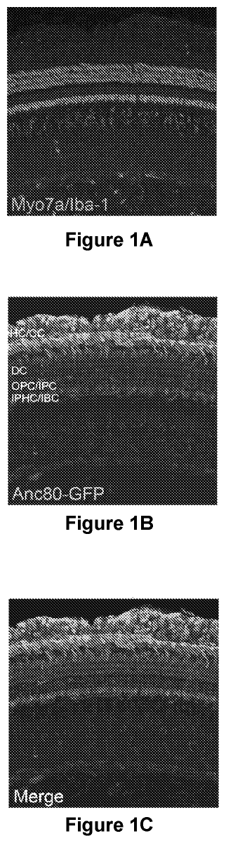

The cochlear tissue from the treated macaque was processed for immunofluorescence analysis using Myo7a as a marker for hair cells and Iba-1 as a marker for macrophages. The middle turn is representative of the entire sensory epithelium. The data in FIGS. 1A-1C show clear GFP expression in both the hair cells and the supporting cells, including the following supporting cell subtypes: Hensen's cells (HC), Claudius cells (CC), Dieter cells (DC), inner and outer pillar cells (OPC/IPC), inner border cells, and inner phalangeal cells (IPHC/IBC). These data demonstrate successful Anc80-GFP AAV vector transduction into different cell types of the inner ear sensory epithelium, and the resulting expression of the encoded reporter gene (GFP) in these different cell types. These data indicate the present claimed compositions including a single AAV vector or two or more AAV vectors can be used to express a gene in hair cells and supporting cells, and can be used to repair a mutation in a gene in hair cells and supporting cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A composition comprising at least two different nucleic acid vectors, wherein:
each of the at least two different adeno-associated virus (AAV) vectors comprises a coding sequence that encodes a different portion of a supporting cell target protein, each of the encoded portions being at least 30 amino acid residues in length, wherein the amino acid sequence of each of the encoded portions may optionally partially overlap with the amino acid sequence of a different one of the encoded portions;
no single vector of the at least two different vectors encodes the full-length supporting cell target protein;
at least one of the coding sequences comprises a nucleotide sequence spanning two neighboring exons of the supporting cell target genomic DNA, and lacks an intronic sequence between the two neighboring exons; and
when introduced into a primate cell the at least two different vectors undergo concatamerization or homologous recombination with each other, thereby forming a recombined nucleic acid that encodes a full-length supporting cell target protein.

2. The composition of claim 1, wherein the amino acid sequence of one of the encoded portions overlaps with the amino acid sequence of a different one of the encoded portions.

3. The composition of claim 2, wherein the amino acid sequence of each of the encoded portions partially overlaps with the amino acid sequence of a different encoded portion.

4. The composition of claim 2, wherein the overlapping amino acid sequence is between 30 amino acid residues to about 390 amino acid residues in length.

5. The composition of any one of claims 1-4, wherein each of the at least two different vectors comprises a different segment of an intron, wherein the intron comprises the nucleotide sequence of an intron that is present in a supporting cell target genomic DNA, and wherein the two different segments overlap in sequence by at least 100 nucleotides.

6. The composition of claim 5, wherein the two different segments overlap in sequence by about 30 nucleotides to about 390 nucleotides.

7. The composition of any one of claims 1-6, wherein the nucleotide sequence of each of the at least two different vectors is between about 30 nucleotides to about 390 nucleotides in length.

8. The composition of claim 7, wherein the nucleotide sequence of each of the at least two different vectors is between 30 nucleotides to 340 nucleotides in length.

9. The composition of any one of claims 1-8, wherein the number of different vectors in the composition is two.

10. The composition of claim 9, wherein a first of the two different vectors comprises a coding sequence that encodes an N-terminal portion of the supporting cell target protein.

11. The composition of claim 10, wherein the N-terminal portion of the supporting cell target protein is between about 30 amino acids to about 390 amino acids in length.

12. The composition of claim 11, wherein the N-terminal portion of the supporting cell target protein is between about 30 amino acids to about 340 amino acids in length.

13. The composition of any one of claims 10-12, wherein the first vector further comprises one or both of a promoter and a Kozak sequence.

14. The composition of claim 13, wherein the first vector comprises a promoter that is an inducible promoter, a constitutive promoter, or a tissue-specific promoter.

15. The composition of any one of claims 10-14, wherein the second of the two different vectors comprises a coding sequence that encodes a C-terminal portion of the supporting cell target protein.

16. The composition of claim 15, wherein the C-terminal portion of the supporting cell target protein is between 30 amino acids to about 390 amino acids in length.

17. The composition of claim 16, wherein the C-terminal portion of the supporting cell target protein is between 30 amino acids to about 340 amino acids in length.

18. The composition of any one of claims 15-17, wherein the second vector further comprises a poly(dA) sequence.

19. A composition comprising two different nucleic acid vectors, wherein:
a first nucleic acid vector of the two different nucleic acid vectors comprises a promoter, a first coding sequence that encodes an N-terminal portion of a supporting cell target protein positioned 3′ of the promoter, and a splicing donor signal sequence positioned at the 3′ end of the first coding sequence; and
a second nucleic acid vector of the two different nucleic acid vectors comprises a splicing acceptor signal sequence, a second coding sequence that encodes a C-terminal portion of a supporting cell target protein positioned at the 3′ end of the splicing acceptor signal sequence, and a polyadenylation sequence at the 3′ end of the second coding sequence;
wherein each of the encoded portions is at least 30 amino acid residues in length, wherein the amino acid sequences of the encoded portions do not overlap, wherein no single vector of the two different vectors encodes the full-length supporting cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length supporting cell target protein.

20. The composition of claim 19, wherein at least one of the coding sequences comprises a nucleotide sequence spanning two neighboring exons of a supporting cell target genomic DNA, and lacks an intronic sequence between the neighboring exons.

21. A composition comprising:
a first nucleic acid vector comprising a promoter, a first coding sequence that encodes an N-terminal portion of a supporting cell target protein positioned 3′ of the promoter, a splicing donor signal sequence positioned at the 3′ end of the first coding sequence, and a first detectable marker gene positioned 3′ of the splicing donor signal sequence; and
a second nucleic acid vector, different from the first nucleic acid vector, comprising a second detectable marker gene, a splicing acceptor signal sequence positioned 3′ of the second detectable marker gene, a second coding sequence that encodes a C-terminal portion of a supporting cell target protein positioned at the 3′ end of the splicing acceptor signal sequence, and a polyadenylation sequence positioned at the 3′ end of the second coding sequence;
wherein each of the encoded portions is at least 30 amino acid residues in length, wherein the respective amino acid sequences of the encoded portions do not overlap with each other, wherein no single vector of the two different vectors encodes the full-length supporting cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length supporting cell target protein.

22. The composition of claim 21, wherein at least one of the coding sequences comprises a nucleotide sequence spanning two neighboring exons of a supporting cell target genomic DNA, and lacks an intronic sequence between the neighboring exons.

23. The composition of claim 21, wherein the first or second detectable marker gene is alkaline phosphatase.

24. The composition of claim 21 or 23, wherein the first and second detectable marker genes are the same.

25. A composition comprising:
a first nucleic acid vector comprising a promoter, a first coding sequence that encodes an N-terminal portion of a supporting cell target protein positioned 3′ to the promoter, a splicing donor signal sequence positioned at the 3′ end of the first coding sequence, and a F1 phage recombinogenic region positioned 3′ to the splicing donor signal sequence; and
a second nucleic acid vector, different from the first nucleic acid vector, comprising a second F1 phage recombinogenic region, a splicing acceptor signal sequence positioned 3′ of the second F1 phage recombinogenic region, a second coding sequence that encodes a C-terminal portion of a supporting cell target protein positioned at the 3′ end of the splicing acceptor signal sequence, and a polyadenylation sequence positioned at the 3′ end of the second coding sequence;
wherein each of the encoded portions is at least 30 amino acid residues in length, wherein the respective amino acid sequences of the encoded portions do not overlap with each other, wherein no single vector of the two different vectors encodes the full-length supporting cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length supporting cell target protein.

26. The composition of claim 25, wherein at least one of the coding sequences comprises a nucleotide sequence spanning two neighboring exons of a supporting cell target genomic DNA, and lacks an intronic sequence between the two neighboring exons.

27. A composition comprising a single adeno-associated virus (AAV) vector, wherein the single AAV vector that comprises a nucleic acid sequence that encodes a supporting cell target protein; and
when introduced into a primate cell, a nucleic acid encoding a full-length supporting cell target protein is generated at the locus of the supporting cell target gene, and the primate expresses the supporting cell target protein.

28. The composition of any one of claims 1-27, wherein the supporting cell target gene is gap junction protein beta 2 (GJB2).

29. The composition of any one of claims 1-27, wherein the supporting cell target gene is solute carrier family 26 member 4 (SLC26A4).

30. A composition comprising at least two different nucleic acid vectors, wherein:
each of the at least two different adeno-associated virus (AAV) vectors comprises a coding sequence that encodes a different portion of a hair cell target protein, each of the encoded portions being at least 30 amino acid residues in length, wherein the amino acid sequence of each of the encoded portions may optionally partially overlap with the amino acid sequence of a different one of the encoded portions;
no single vector of the at least two different vectors encodes the full-length hair cell target protein;
at least one of the coding sequences comprises a nucleotide sequence spanning two neighboring exons of the hair cell target genomic DNA, and lacks an intronic sequence between the two neighboring exons; and
when introduced into a primate cell the at least two different vectors undergo concatamerization or homologous recombination with each other, thereby forming a recombined nucleic acid that encodes a full-length hair cell target protein.

31. The composition of claim 30, wherein the amino acid sequence of one of the encoded portions overlaps with the amino acid sequence of a different one of the encoded portions.

32. The composition of claim 31, wherein the amino acid sequence of each of the encoded portions partially overlaps with the amino acid sequence of a different encoded portion.

33. The composition of claim 31, wherein the overlapping amino acid sequence is between 30 amino acid residues to about 390 amino acid residues in length.

34. The composition of any one of claims 30-33, wherein each of the at least two different vectors comprises a different segment of an intron, wherein the intron comprises the nucleotide sequence of an intron that is present in a hair cell target genomic DNA, and wherein the two different segments overlap in sequence by at least 100 nucleotides.

35. The composition of claim 34, wherein the two different segments overlap in sequence by about 30 nucleotides to about 390 nucleotides.

36. The composition of any one of claims 30-35, wherein the nucleotide sequence of each of the at least two different vectors is between about 30 nucleotides to about 390 nucleotides in length.

37. The composition of claim 36, wherein the nucleotide sequence of each of the at least two different vectors is between 30 nucleotides to 340 nucleotides in length.

38. The composition of any one of claims 30-37, wherein the number of different vectors in the composition is two.

39. The composition of claim 38, wherein a first of the two different vectors comprises a coding sequence that encodes an N-terminal portion of the hair cell target protein.

40. The composition of claim 39, wherein the N-terminal portion of the hair cell target protein is between about 30 amino acids to about 390 amino acids in length.

41. The composition of claim 40, wherein the N-terminal portion of the hair cell target protein is between about 30 amino acids to about 340 amino acids in length.

42. The composition of any one of claims 39-41, wherein the first vector further comprises one or both of a promoter and a Kozak sequence.

43. The composition of claim 42, wherein the first vector comprises a promoter that is an inducible promoter, a constitutive promoter, or a tissue-specific promoter.

44. The composition of any one of claims 39-41, wherein the second of the two different vectors comprises a coding sequence that encodes a C-terminal portion of the hair cell target protein.

45. The composition of claim 44, wherein the C-terminal portion of the hair cell target protein is between 30 amino acids to about 390 amino acids in length.

46. The composition of claim 45, wherein the C-terminal portion of the hair cell target protein is between 30 amino acids to about 340 amino acids in length.

47. The composition of any one of claims 44-46, wherein the second vector further comprises a poly(dA) sequence.

48. A composition comprising two different nucleic acid vectors, wherein:
a first nucleic acid vector of the two different nucleic acid vectors comprises a promoter, a first coding sequence that encodes an N-terminal portion of a hair cell target protein positioned 3′ of the promoter, and a splicing donor signal sequence positioned at the 3′ end of the first coding sequence; and
a second nucleic acid vector of the two different nucleic acid vectors comprises a splicing acceptor signal sequence, a second coding sequence that encodes a C-terminal portion of a hair cell target protein positioned at the 3′ end of the splicing acceptor signal sequence, and a polyadenylation sequence at the 3′ end of the second coding sequence;
wherein each of the encoded portions is at least 30 amino acid residues in length, wherein the amino acid sequences of the encoded portions do not overlap, wherein no single vector of the two different vectors encodes the full-length hair cell target protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length hair cell target protein.

49. The composition of claim 48, wherein at least one of the coding sequences comprises a nucleotide sequence spanning two neighboring exons of a hair cell target genomic DNA, and lacks an intronic sequence between the neighboring exons.

50. A composition comprising:
a first nucleic acid vector comprising a promoter, a first coding sequence that encodes an N-terminal portion of a hair cell target protein positioned 3′ of the promoter, a splicing donor signal sequence positioned at the 3′ end of the first coding sequence, and a first detectable marker gene positioned 3′ of the splicing donor signal sequence; and
a second nucleic acid vector, different from the first nucleic acid vector, comprising a second detectable marker gene, a splicing acceptor signal sequence positioned 3′ of the second detectable marker gene, a second coding sequence that encodes a C-terminal portion of a hair cell target protein positioned at the 3′ end of the splicing acceptor signal sequence, and a polyadenylation sequence positioned at the 3′ end of the second coding sequence;
wherein each of the encoded portions is at least 30 amino acid residues in length, wherein the respective amino acid sequences of the encoded portions do not overlap with each other, wherein no single vector of the two different vectors encodes the full-length hair cell target protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length hair cell target protein.

51. The composition of claim 50, wherein at least one of the coding sequences comprises a nucleotide sequence spanning two neighboring exons of a hair cell target genomic DNA, and lacks an intronic sequence between the neighboring exons.

52. The composition of claim 50, wherein the first or second detectable marker gene is alkaline phosphatase.

53. The composition of claim 50 or 52, wherein the first and second detectable marker genes are the same.

54. A composition comprising:
a first nucleic acid vector comprising a promoter, a first coding sequence that encodes an N-terminal portion of a hair cell target protein positioned 3′ to the promoter, a splicing donor signal sequence positioned at the 3′ end of the first coding sequence, and a F1 phage recombinogenic region positioned 3′ to the splicing donor signal sequence; and
a second nucleic acid vector, different from the first nucleic acid vector, comprising a second F1 phage recombinogenic region, a splicing acceptor signal sequence positioned 3′ of the second F1 phage recombinogenic region, a second coding sequence that encodes a C-terminal portion of a hair cell target protein positioned at the 3′ end of the splicing acceptor signal sequence, and a polyadenylation sequence positioned at the 3′ end of the second coding sequence;
wherein each of the encoded portions is at least 30 amino acid residues in length, wherein the respective amino acid sequences of the encoded portions do not overlap with each other, wherein no single vector of the two different vectors encodes the full-length hair cell target protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length hair cell target protein.

55. The composition of claim 54, wherein at least one of the coding sequences comprises a nucleotide sequence spanning two neighboring exons of a hair cell target genomic DNA, and lacks an intronic sequence between the two neighboring exons.

56. The composition of any one of claims 30-55, wherein the hair cell target gene is an OTOF gene, a TRIOBP gene, or a STRC gene.

57. A composition comprising a single adeno-associated virus (AAV) vector, wherein the single AAV vector that comprises a nucleic acid sequence that encodes a hair cell target protein; and
when introduced into a primate cell, a nucleic acid encoding a full-length hair cell target protein is generated at the locus of the hair cell target gene, and the primate expresses the hair cell target protein.

58. The composition of claim 57, wherein the hair cell target gene is a PJVK gene, an OTOF gene, a NDP gene, a CLRN1 gene, a TRIOBP gene, a STRC gene, a TMC1 gene, a VEGF gene or a HSPA1A gene.

59. The composition of any one of claims 1-58, further comprising a pharmaceutically acceptable excipient.

60. A kit comprising a composition of any one of claims 1-59.

61. A kit of claim 60, further comprising a pre-loaded syringe comprising the composition.

62. A method of correcting a supporting cell target gene defect in a supporting cell of an inner ear in a primate, the method comprising:
administering to the inner ear of the primate a therapeutically effective amount of a composition of any one of claims 1-29,
wherein the administering repairs the supporting cell target gene defect in the supporting cell of the inner ear of the primate.

63. A method of increasing the expression level of a supporting cell target protein in a supporting cell of an inner ear of a primate, the method comprising administering to the inner ear of the primate a therapeutically effective amount of a composition of any one of claims 1-29,
wherein the administering results in an increase in the expression level of the supporting cell target protein in the supporting cell of the inner ear of the primate.

64. The method of claim 63, wherein the primate has previously been determined to have a defective supporting cell target gene.

65. A method of treating non-syndromic sensorineural hearing loss in a primate identified as having a defective supporting cell target gene, the method comprising:
administering to the inner ear of the primate a therapeutically effective amount of a composition of any one of claims 1-29.

66. The method of claim 65, further comprising, prior to the administering step, determining that the primate has a defective supporting cell target gene.

67. A method of restoring hearing in a primate identified or diagnosed as having hearing loss, the method comprising:
administering to the inner ear of the primate a therapeutically effective amount of a composition of any one of claims 1-29.

68. The method of claim 67, further comprising, prior to the administering step, determining that the primate has a defective supporting cell target gene.

69. A method of restoring synapses and/or preserving spiral ganglion nerves in a primate identified or diagnosed as having an inner ear disorder, the method comprising:
administering to the inner ear of the primate a therapeutically effective amount of a composition of any one of claims 1-29.

70. The method of claim 69, further comprising, prior to the administering step, determining that the primate has a defective supporting cell target gene.

71. A method comprising administering to an inner ear of a primate a therapeutically effective amount of a composition of any one of claims 1-29.

72. The method of claim 71, wherein the primate has been previously identified as having a defective supporting cell target gene.

73. A method of correcting a hair cell target gene defect in a hair cell of an inner ear in a primate, the method comprising:
administering to the inner ear of the primate a therapeutically effective amount of a composition of any one of claims 30-59,
wherein the administering repairs the hair cell target gene defect in the hair cell of the inner ear of the primate.

74. A method of increasing the expression level of a hair cell target protein in a hair cell of an inner ear of a primate, the method comprising administering to the inner ear of the primate a therapeutically effective amount of a composition of any one of claims 30-59,
wherein the administering results in an increase in the expression level of the hair cell target protein in the hair cell of the inner ear of the primate.

75. The method of claim 74, wherein the primate has previously been determined to have a defective hair cell target gene.

76. A method of treating non-syndromic sensorineural hearing loss in a primate identified as having a defective hair cell target gene, the method comprising:
administering to the inner ear of the primate a therapeutically effective amount of a composition of any one of claims 30-59.

77. The method of claim 76, further comprising, prior to the administering step, determining that the primate has a defective hair cell target gene.

78. A method of restoring hearing in a primate identified or diagnosed as having hearing loss, the method comprising:
administering to the inner ear of the primate a therapeutically effective amount of a composition of any one of claims 30-59.

79. The method of claim 78, further comprising, prior to the administering step, determining that the primate has a defective hair cell target gene.

80. A method of restoring synapses and/or preserving spiral ganglion nerves in a primate identified or diagnosed as having an inner ear disorder, the method comprising:
administering to the inner ear of the primate a therapeutically effective amount of a composition of any one of claims 30-59.

81. The method of claim 80, further comprising, prior to the administering step, determining that the primate has a defective hair cell target gene.

82. A method comprising administering to an inner ear of a primate a therapeutically effective amount of a composition of any one of claims 30-59.

83. The method of claim 82, wherein the primate has been previously identified as having a defective hair cell target gene.

84. The composition of any one of claim 1-26 or 30-56, wherein the second nucleic acid vector further comprises a destabilizing sequence.

85. The composition of claim 84, wherein the second nucleic acid vector further comprises a FKBP12 destabilizing sequence.

86. The composition of any one of claim 27-29 or 57-59, wherein the nucleic acid vector further comprises a destabilizing sequence.

87. The composition of claim 86, wherein the nucleic acid vector comprises a FKBP12 destabilizing sequence.
